SEQUENCE LISTING (308 sequences)

SEQ ID NO: 1 - protein, 40 aa, Homo sapiens
YKLVVVGAGGVGKSALTIQLIQNHFVDEYDPTIEDSYRKQ

SEQ ID NO: 2 - protein, 40 aa, Homo sapiens; identical to SEQ ID NO: 1

SEQ ID NO: 3 - protein, 40 aa, Homo sapiens; identical to SEQ ID NO: 1

SEQ ID NO: 4 - protein, 40 aa, Homo sapiens
HKLVVVGGGGVGKSALTIQFIQSYFVSDYDPTIEDSYTKI

SEQ ID NO: 5 - protein, 38 aa, Homo sapiens
MRILMVGLDAAGKTTILYKLKLGEIVTTIPTIGFNVET

SEQ ID NO: 6 - protein, 38 aa, Homo sapiens; identical to SEQ ID NO: 5

SEQ ID NO: 7 - protein, 38 aa, Homo sapiens; identical to SEQ ID NO: 5

SEQ ID NO: 8 - protein, 38 aa, Homo sapiens; identical to SEQ ID NO: 5

SEQ ID NO: 9 - protein, 38 aa, Homo sapiens
MRILMLGLDAAGKTTILYKLKLGQSVTTIPTVGFNVET

SEQ ID NO: 10 - protein, 38 aa, Homo sapiens
IRVVTLGLDGAGKTTILFKLKQDEFMQPIPTIGFNVET

SEQ ID NO: 11 - protein, 38 aa, Homo sapiens
MRILILGLDGAGKTTILYRLQVGEVVTTIPTIGFNVET

SEQ ID NO: 12 - protein, 38 aa, Homo sapiens
HKVIIVGLDNAGKTTILYQFSMNEVVHTSPTIGSNVEE

SEQ ID NO: 13 - protein, 38 aa, Homo sapiens
HKVIIVGLDNAGKTTILYQFLMNEVVHTSPTIGSNVEE

SEQ ID NO: 14 - protein, 38 aa, Homo sapiens
LRLLMLGLDNAGKTTILKKFNGEDIDTISPTLGFNIKT

SEQ ID NO: 15 - protein, 38 aa, Homo sapiens
VRILLLGLDNAGKTTLLKQLASEDISHITPTQGFNIKS

SEQ ID NO: 16 - protein, 40 aa, Homo sapiens
VHVLCLGLDNSGKTTIINKLKPSNAQSQNILPTIGFSIEK

SEQ ID NO: 17 - protein, 38 aa, Homo sapiens
FHIVILGLDCAGKTTVLYRLQFNEFVNTVPTKGFNTEK

SEQ ID NO: 18 - protein, 38 aa, Homo sapiens
LHIVMLGLDSAGKTTVLYRLKFNEFVNTVPTIGFNTEK

SEQ ID NO: 19 - protein, 38 aa, Homo sapiens
LHVVVIGLDSAGKTSLLYRLKFKEFVQSVPTKGFNTEK

SEQ ID NO: 20 - protein, 38 aa, Homo sapiens
AQVVMMGLDSAGKTTLLYKLKGHQLVETLPTVGFNVEP

SEQ ID NO: 21 - protein, 38 aa, Homo sapiens
AQVLLLGLDSAGKSTLLYKLKLAKDITTIPTIGFNVEM

SEQ ID NO: 22 - protein, 46 aa, Homo sapiens
YCILILGLDNAGKTTFLEQSKTRFNKNYKGMSLSKITTTVGLNIGT

SEQ ID NO: 23 - protein, 38 aa, Homo sapiens
VTLLMVGLDNAGKTATAKGIQGEYPEDVAPTVGFSKIN

SEQ ID NO: 24 - protein, 39 aa, Homo sapiens
KQILVLGLDGAGKTSVLHSLASNRVQHSVAPTQGFHAVC

SEQ ID NO: 25 - protein, 39 aa, Homo sapiens
REVLVLGLDGAGKSTFLRVLSGKPPLEGHIPTWGFNSVR

SEQ ID NO: 26 - protein, 38 aa, Homo sapiens
GKLVFLGLDNAGKTTLLHMLKDDRLGQHVPTLHPTSEE

SEQ ID NO: 27 - protein, 38 aa, Homo sapiens; identical to SEQ ID NO: 26

SEQ ID NO: 28 - protein, 39 aa, Homo sapiens
MELTLVGLQYSGKTTFVNVIASGQFNEDMIPTVGFNMRK

SEQ ID NO: 29 - protein, 39 aa, Homo sapiens
MELTLVGLQYSGKTTFVNVIASGQFSEDMIPTVGFNMRK

SEQ ID NO: 30 - protein, 48 aa, Homo sapiens
GMCLLLGATGVGKTLLVKRLQEVSSRDGKGDLGEPPPTRPTVGTNLTD

SEQ ID NO: 31 - protein, 40 aa, Homo sapiens
KKLVIVGDGACGKTCLLIVFSKDQFPEVYVPTVFENYVAD

SEQ ID NO: 32 - protein, 40 aa, Homo sapiens
KKLVIVGDGACGKTCLLIVFSKDQFPEVYVPTVFENYIAD

SEQ ID NO: 33 - protein, 40 aa, Homo sapiens
KKLVVVGDGACGKTCLLIVFSKDEFPEVYVPTVFENYVAD

SEQ ID NO: 34 - protein, 40 aa, Homo sapiens
CKIVVVGDAECGKTALLQVFAKDAYPGSYVPTVFENYTAS

SEQ ID NO: 35 - protein, 40 aa, Homo sapiens
CKIVVVGDSQCGKTALLHVFAKDCFPENYVPTVFENYTAS

SEQ ID NO: 36 - protein, 40 aa, Homo sapiens
CKLVLVGDVQCGKTAMLQVLAKDCYPETYVPTVFENYTAC

SEQ ID NO: 37 - protein, 40 aa, Homo sapiens
VKVVLVGDGGCGKTSLLMVFADGAFPESYTPTVFERYMVN

SEQ ID NO: 38 - protein, 40 aa, Homo sapiens
LKIVIVGDGGCGKTSLLMVYSQGSFPEHYAPSVFEKYTAS

SEQ ID NO: 39 - protein, 40 aa, Homo sapiens
IKCVVVGDGAVGKTCLLISYTTNAFPGEYIPTVFDNYSAN

SEQ ID NO: 40 - protein, 40 aa, Homo sapiens; identical to SEQ ID NO: 39

SEQ ID NO: 41 - protein, 40 aa, Homo sapiens; identical to SEQ ID NO: 39

SEQ ID NO: 42 - protein, 40 aa, Homo sapiens
IKCVVVGDGAVGKTCLLICYTTNAFPKEYIPTVFDNYSAQ

SEQ ID NO: 43 - protein, 40 aa, Homo sapiens
LKCVVVGDGAVGKTCLLMSYANDAFPEEYVPTVFDHYAVS

SEQ ID NO: 44 - protein, 40 aa, Homo sapiens
LKCVVVGDGAVGKTCLLMSYANDAFPEEYVPTVFDHYAVT

SEQ ID NO: 45 - protein, 40 aa, Homo sapiens
IKCVVVGDGAVGKTCLLISYTTNKFPSEYVPTVFDNYAVT

SEQ ID NO: 46 - protein, 40 aa, Homo sapiens
IKCVLVGDGAVGKSSLIVSYTCNGYPARYRPTALDTFSVQ

SEQ ID NO: 47 - protein, 40 aa, Homo sapiens
VKCVLVGDGAVGKTSLVVSYTTNGYPTEYIPTAFDNFSAV

SEQ ID NO: 48 - protein, 40 aa, Homo sapiens
IKCVLVGDSAVGKTSLLVRFTSETFPEAYKPTVYENTGVD

SEQ ID NO: 49 - protein, 47 aa, Homo sapiens
IKCVVVGDNAVGKTRLICARACNTTLTQYQLLATHVPTVWAIDQYRV

SEQ ID NO: 50 - protein, 47 aa, Homo sapiens
IKCVVVGDNAVGKTRLICARACNATLTQYQLLATHVPTVWAIDQYRV

SEQ ID NO: 51 - protein, 40 aa, Homo sapiens
YRLVVVGGGGVGKSALTIQFIQSYFVTDYDPTIEDSYTKQ

SEQ ID NO: 52 - protein, 40 aa, Homo sapiens
YKLVVVGDGGVGKSALTIQFFQKIFVPDYDPTIEDSYLKH

SEQ ID NO: 53 - protein, 40 aa, Homo sapiens
YKLVMLGAGGVGKSAMTMQFISHRFPEDHDPTIEDAYKIR

SEQ ID NO: 54 - protein, 40 aa, Homo sapiens
YKVVMLGAGGVGKSAMTMQFISHQFPDYHDPTIEDAYKTQ

SEQ ID NO: 55 - protein, 40 aa, Homo sapiens
YKLVVLGSGGVGKSALTVQFVQGIFVEKYDPTIEDSYRKQ

SEQ ID NO: 56 - protein, 40 aa, Homo sapiens; identical to SEQ ID NO: 55

SEQ ID NO: 57 - protein, 40 aa, Homo sapiens
YKVVVLGSGGVGKSALTVQFVTGTFIEKYDPTIEDFYRKE

SEQ ID NO: 58 - protein, 40 aa, Homo sapiens; identical to SEQ ID NO: 57

SEQ ID NO: 59 - protein, 40 aa, Homo sapiens
YKVVVLGSGGVGKSALTVQFVTGSFIEKYDPTIEDFYRKE

SEQ ID NO: 60 - protein, 40 aa, Homo sapiens
HKVIMVGSGGVGKSALTLQFMYDEFVEDYEPTKADSYRKK

SEQ ID NO: 61 - protein, 40 aa, Homo sapiens; identical to SEQ ID NO: 60

SEQ ID NO: 62 - protein, 40 aa, Homo sapiens
YKAVVVGASGVGKSALTIQLNHQCFVEDHDPTIQDSYWKE

SEQ ID NO: 63 - protein, 42 aa, Homo sapiens
YRVVLIGEQGVGKSTLANIFAGVHDSMDSDCEVLGEDTYERT

SEQ ID NO: 64 - protein, 39 aa, Homo sapiens
YKVLLLGAPGVGKSALARIFGGVEDGPEAEAAGHTYDRS

SEQ ID NO: 65 - protein, 40 aa, Homo sapiens
YRVVLLGDPGVGKTSLASLFAGKQERDLHEQLGEDVYERT

SEQ ID NO: 66 - protein, 41 aa, Homo sapiens
FKVMLVGESGVGKSTLAGTFGGLQGDSAHEPENPEDTYERR

SEQ ID NO: 67 - protein, 40 aa, Homo sapiens
VKLAIFGRAGVGKSALVVRFLTKRFIWEYDPTLESTYRHQ

SEQ ID NO: 68 - protein, 40 aa, Homo sapiens
VNLAILGRRGAGKSALTVKFLTKRFISEYDPNLEDTYSSE

SEQ ID NO: 69 - protein, 40 aa, Homo sapiens
IKLAVLGAGRVGKSAMIVRFLTKRFIGDYEPNTGKLYSRL

SEQ ID NO: 70 - protein, 40 aa, Homo sapiens
VKIAVVGASGVGKTALVVRFLTKRFIGDYERNAGNLYTRQ

SEQ ID NO: 71 - protein, 40 aa, Homo sapiens
LHLKYNEKSVSVTKALTVRFLTKRFIGEYASNFESIYKKH

SEQ ID NO: 72 - protein, 40 aa, Homo sapiens
YRVVVFGAGGVGKSSLVLRFVKGTFRDTYIPTIEDTYRQV

SEQ ID NO: 73 - protein, 40 aa, Homo sapiens
YRVAVFGAGGVGKSSLVLRFVKGTFRESYIPTVEDTYRQV

SEQ ID NO: 74 - protein, 40 aa, Homo sapiens
YRVVVVGTAGVGKSTLLHKWASGNFRHEYLPTIENTYCQL

SEQ ID NO: 75 - protein, 40 aa, Homo sapiens
YRMVILGSSKVGKTAIVSRFLTGRFEDAYTPTIEDFHRKF

SEQ ID NO: 76 - protein, 40 aa, Homo sapiens
YRMVVLGASRVGKSSIVSRFLNGRFEDQYTPTIEDFHRKV

SEQ ID NO: 77 - protein, 41 aa, Homo sapiens
LRVAVLGAPGVGKTAIIRQFLFGDYPERHRPTDGPRLYRPA

SEQ ID NO: 78 - protein, 41 aa, Homo sapiens
YRVAVLGARGVGKSAIVRQFLYNEFSEVCVPTTARRLYLPA

SEQ ID NO: 79 - protein, 40 aa, Homo sapiens
RKIAILGYRSVGKSSLTIQFVEGQFVDSYDPTIENTFTKL

SEQ ID NO: 80 - protein, 40 aa, Homo sapiens
RKVVILGYRCVGKTSLAHQFVEGEFSEGYDPTVENTYSKI

SEQ ID NO: 81 - protein, 42 aa, Homo sapiens
CKVVVCGLLSVGKTAILEQLLYGNHTIGMEDCETMEDVYMAS

SEQ ID NO: 82 - protein, 42 aa, Homo sapiens
CKVVVCGQASVGKTSILEQLLYGNHVVGSEMIETQEDIYVGS

SEQ ID NO: 83 - protein, 41 aa, Homo sapiens
LKFLLVGDRDVGKSEILESLQDGAAESPYSHLGGIDYKTTT

SEQ ID NO: 84 - protein, 41 aa, Homo sapiens
LKFLLVGDSDVGKGEILASLQDGAAESPYGHPAGIDYKTTT

SEQ ID NO: 85 - protein, 41 aa, Homo sapiens
LKFLLVGDSDVGKGEILESLQDGAAESPYAYSNGIDYKTTT

SEQ ID NO: 86 - protein, 41 aa, Homo sapiens
FKLLLIGDSGVGKSCLLLRFADDTYTESYISTIGVDFKIRT

SEQ ID NO: 87 - protein, 41 aa, Homo sapiens; identical to SEQ ID NO: 86

SEQ ID NO: 88 - protein, 41 aa, Homo sapiens
FKLLIIGDSGVGKSSLLLRFADNTFSGSYITTIGVDFKIRT

SEQ ID NO: 89 - protein, 41 aa, Homo sapiens
FKILIIGNSSVGKTSFLFRYADDSFTPAFVSTVGIDFKVKT

SEQ ID NO: 90 - protein, 41 aa, Homo sapiens
FKLLIIGNSSVGKTSFLFRYADDSFTSAFVSTVGIDFKVKT

SEQ ID NO: 91 - protein, 41 aa, Homo sapiens
FKLLIIGNSSVGKTSFLFRYADDTFTPAFVSTVGIDFKVKT

SEQ ID NO: 92 - protein, 41 aa, Homo sapiens
FKLLLIGNSSVGKTSFLFRYADDSFTPAFVSTVGIDFKVKT

SEQ ID NO: 93 - protein, 41 aa, Homo sapiens
FKLLLIGDSGVGKTCVLFRFSEDAFNSTFISTIGIDFKIRT

SEQ ID NO: 94 - protein, 41 aa, Homo sapiens
FKLLLIGDSGVGKTCLLFRFSEDAFNTTFISTIGIDFKIRT

SEQ ID NO: 95 - protein, 41 aa, Homo sapiens
FKLLLIGDSGVGKTCVLFRFSDDAFNTTFISTIGIDFKIKT

SEQ ID NO: 96 - protein, 41 aa, Homo sapiens
FKLLLIGDSGVGKTCLIIRFAEDNFNNTYISTIGIDFKIRT

SEQ ID NO: 97 - protein, 41 aa, Homo sapiens
LQVIIIGSRGVGKTSLMERFTDDTFCEACKSTVGVDFKIKT

SEQ ID NO: 98 - protein, 41 aa, Homo sapiens
LKILIIGESGVGKSSLLLRFTDDTFDPELAATIGVDFKVKT

SEQ ID NO: 99 - protein, 41 aa, Homo sapiens
FKIILIGDSNVGKTCVVQHFKSGVYTETQQNTIGVDFTVRS

SEQ ID NO: 100 - protein, 41 aa, Homo sapiens
FKLVLVGDASVGKTCVVQRFKTGAFSERQGSTIGVDFTMKT

SEQ ID NO: 101 - protein, 41 aa, Homo sapiens
FKIVLIGNAGVGKTCLVRRFTQGLFPPGQGATIGVDFMIKT

SEQ ID NO: 102 - protein, 41 aa, Homo sapiens
FKIIVIGDSNVGKTCLTFRFCGGTFPDKTEATIGVDFREKT

SEQ ID NO: 103 - protein, 41 aa, Homo sapiens
FKIIVIGDSNVGKTCLTYRFCAGRFPDRTEATIGVDFRERA

SEQ ID NO: 104 - protein, 41 aa, Homo sapiens
FKYIIIGDTGVGKSCLLLQFTDKRFQPVHDLTIGVEFGARM

SEQ ID NO: 105 - protein, 41 aa, Homo sapiens; identical to SEQ ID NO: 104

SEQ ID NO: 106 - protein, 41 aa, Homo sapiens
FKFLVIGNAGTGKSCLLHQFIEKKFKDDSNHTIGVEFGSKI

SEQ ID NO: 107 - protein, 41 aa, Homo sapiens
FKFLVIGSAGTGKSCLLHQFIENKFKQDSNHTIGVEFGSRV

SEQ ID NO: 108 - protein, 41 aa, Homo sapiens
FKYIIIGDMGVGKSCLLHQFTEKKFMADCPHTIGVEFGTRI

SEQ ID NO: 109 - protein, 41 aa, Homo sapiens
FKVVLIGDSGVGKSNLLSRFTRNEFNLESKSTIGVEFATRS

SEQ ID NO: 110 - protein, 41 aa, Homo sapiens; identical to SEQ ID NO: 109

SEQ ID NO: 111 - protein, 41 aa, Homo sapiens
FKVVLIGESGVGKTNLLSRFTRNEFSHDSRTTIGVEFSTRT

SEQ ID NO: 112 - protein, 45 aa, Homo sapiens
FRLIVIGDSTVGKSCLLHRFTQGRFPGLRSPACDPTVGVDFFSRL

SEQ ID NO: 113 - protein, 41 aa, Homo sapiens
FRLIVIGDSTVGKSCLIRRFTEGRFAQVSDPTVGVDFFSRL

SEQ ID NO: 114 - protein, 46 aa, Homo sapiens
FRVALLGDAAVGKTSLLRSYVAGAPGAPEPEPEPEPTVGAECYRRA

SEQ ID NO: 115 - protein, 42 aa, Homo sapiens
FKVMLVGDSGVGKTCLLVRFKDGAFLAGTFISTVGIDFRNKV

SEQ ID NO: 116 - protein, 42 aa, Homo sapiens
HKTILVGDSGVGKTSLLVQFDQGKFIPGSFSATVGIGFTNKV

SEQ ID NO: 117 - protein, 41 aa, Homo sapiens
YKIVLAGDAAVGKSSFLMRLCKNEFRENISATLGVDFQMKT

SEQ ID NO: 118 - protein, 41 aa, Homo sapiens
IKFLALGDSGVGKTSVLYQYTDGKFNSKFITTVGIDFREKR

SEQ ID NO: 119 - protein, 41 aa, Homo sapiens
IKLLALGDSGVGKTTFLYRYTDNKFNPKFITTVGIDFREKR

SEQ ID NO: 120 - protein, 41 aa, Homo sapiens
IKMVVVGNGAVGKSSMIQRYCKGIFTKDYKKTIGVDFLERQ

SEQ ID NO: 121 - protein, 43 aa, Homo sapiens
AAKCILADPAVGKTALAQIFRSDGAHFQKSYTLTTGMDLVVKT

SEQ ID NO: 122 - protein, 40 aa, Homo sapiens
FKVILLGDGGVGKSSLMNRYVTNKFDTQLFHTIGVEFLNK

SEQ ID NO: 123 - protein, 40 aa, Homo sapiens
LKVILLGDGGVGKSSLMNRYVTNKFDSQAFHTIGVEFLNR

SEQ ID NO: 124 - protein, 40 aa, Homo sapiens
LKVIILGDSGVGKTSLMNQYVNKKFSNQYKATIGADFLTK

SEQ ID NO: 125 - protein, 40 aa, Homo sapiens
LKLIIVGAIGVGKTSLPHQYVHKTFYEEYQTTLGASILSK

SEQ ID NO: 126 - protein, 40 aa, Homo sapiens
FKVLVIGELGVGKTSIIKRYVHQLFSQHYRATIGVDFALK

SEQ ID NO: 127 - protein, 40 aa, Homo sapiens
YKLLVIGDLGVGKTSIIKRYVHQNFSSHYRATIGVDFALK

SEQ ID NO: 128 - protein, 40 aa, Homo sapiens
FKVLVVGDAAVGKTSLVQRYSQDSFSKHYKSTVGVDFALK

SEQ ID NO: 129 - protein, 40 aa, Homo sapiens
FKLVLLGESAVGKSSLVLRFVKGQFHEFQESTIGAAFLTQ

SEQ ID NO: 130 - protein, 40 aa, Homo sapiens
FKLVLLGESAVGKSSLVLRFVKGQFHEYQESTIGAAFLTQ

SEQ ID NO: 131 - protein, 40 aa, Homo sapiens; identical to SEQ ID NO: 130

SEQ ID NO: 132 - protein, 40 aa, Homo sapiens
LKVCLLGDTGVGKSSIVWRFVEDSFDPNINPTIGASFMTK

SEQ ID NO: 133 - protein, 40 aa, Homo sapiens
LKVCLLGDTGVGKSSIVCRFVQDHFDHNISPTIGASFMTK

SEQ ID NO: 134 - protein, 41 aa, Homo sapiens
VKVVMLGKEYVGKTSLVERYVHDRFLVGPYQNTIGAAFVAK

SEQ ID NO: 135 - protein, 40 aa, Homo sapiens
FKVVLLGEGCVGKTSLVLRYCENKFNDKHITTLQASFLTK

SEQ ID NO: 136 - protein, 39 aa, Homo sapiens
FKLVLLGSGSVGKSSLALRYVKNDFKSILPTVGCAFFTK

SEQ ID NO: 137 - protein, 40 aa, Homo sapiens
FKLVFLGEQSVGKTSLITRFMYDSFDNTYQATIGIDFLSK

SEQ ID NO: 138 - protein, 40 aa, Homo sapiens
FKLVFLGEQSVAKTSLITRFRYDSFDNTYQAIIGIDFLSK

SEQ ID NO: 139 - protein, 40 aa, Homo sapiens; identical to SEQ ID NO: 137

SEQ ID NO: 140 - protein, 40 aa, Homo sapiens
LKIVVLGDGTSGKTSLTTCFAQETFGKQYKQTIGLDFFLR

SEQ ID NO: 141 - protein, 41 aa, Homo sapiens
FRLLLIGDSGVGKTCLLCRFTDNEFHSSHISTIGVDFKMKT

SEQ ID NO: 142 - protein, 41 aa, Homo sapiens
SKIIVVGDLSVGKTCLINRFCKDTFDKNYKATIGVDFEMER

SEQ ID NO: 143 - protein, 41 aa, Homo sapiens
SKVVVVGDLYVGKTSLIHRFCKNVFDRDYKATIGVDFEIER

SEQ ID NO: 144 - protein, 40 aa, Homo sapiens
FKLVLVGDGGTGKTTFVKRHLTGEFEKKYVATLGVEVHPL

SEQ ID NO: 145 - protein, 41 aa, Homo sapiens
VKIICLGDSAVGKSKLMERFLMDGFQPQQLSTYALTLYKHT

SEQ ID NO: 146 - protein, 41 aa, Homo sapiens; identical to SEQ ID NO: 145

SEQ ID NO: 147 - protein, 46 aa, Homo sapiens
VRILLVGEPRVGKTSLIMSLVSEEFPEEVPPRAEEITIPADVTPER

SEQ ID NO: 148 - protein, 46 aa, Homo sapiens
VRILLLGEAQVGKTSLILSLVGEEFPEEVPPRAEEITIPADVTPEK

SEQ ID NO: 149 - protein, 40 aa, Homo sapiens
SKIVLLGDMNVGKTSLLQRYMERRFPDTVSTVGGAFYLKQ

SEQ ID NO: 150 - protein, 43 aa, Homo sapiens
VKVLVLGDSGVGKSSLVHLLCQNQVLGNPSWTVGCSVDVRVHD

SEQ ID NO: 151 - protein, 38 aa, Homo sapiens
YDLVCIGLTGSGKTSLLSKLCSESPDNVVSTTGFSIKA

SEQ ID NO: 152 - protein, 44 aa, Homo sapiens
AKILFVGPCESGKTVLANFLTESSDITEYSPTQGVRILEFENPH

SEQ ID NO: 153 - protein, 40 aa, Homo sapiens
RAVLLVGLCDSGKTLLFVRLLTGLYRDTQTSITDSCAVYR

SEQ ID NO: 154 - protein, 39 aa, Homo sapiens
MVSGGAGGEGTRVWGTGTPAQRPLVGLGRGLPAEWPEGL

SEQ ID NO: 155 - protein, 50 aa, Homo sapiens
LDTAGQEEYSAMRDQYMRTGEGFLCVFAINNTKSFEDIHQYREQIKRVKD

SEQ ID NO: 156 - protein, 50 aa, Homo sapiens
LDTAGQEEYSAMRDQYMRTGEGFLCVFAINNTKSFEDIHHYREQIKRVKD

SEQ ID NO: 157 - protein, 50 aa, Homo sapiens
LDTAGQEEYSAMRDQYMRTGEGFLCVFAINNSKSFADINLYREQIKRVKD

SEQ ID NO: 158 - protein, 50 aa, Homo sapiens
LDTAGQEEFGAMREQYMRAGHGFLLVFAINDRQSFNEVGKLFTQILRVKD

SEQ ID NO: 159 - protein, 50 aa, Homo sapiens
WDVGGQDKIRPLWRHYFQNTQGLIFVVDSNDRERVNEAREELMRMLAEDE

SEQ ID NO: 160 - protein, 50 aa, Homo sapiens; identical to SEQ ID NO: 159

SEQ ID NO: 161 - protein, 50 aa, Homo sapiens
WDVGGQDRIRPLWKHYFQNTQGLIFVVDSNDRERIQEVADELQKMLLVDE

SEQ ID NO: 162 - protein, 50 aa, Homo sapiens
WDVGGQDKIRPLWRHYFQNTQGLIFVVDSNDRERVQESADELQKMLQEDE

SEQ ID NO: 163 - protein, 50 aa, Homo sapiens
WDVGGQDKIRPLWRHYYTGTQGLIFVVDCADRDRIDEARQELHRIINDRE

SEQ ID NO: 164 - protein, 50 aa, Homo sapiens
WDVGGKHKLRPLWKHYYLNTQAVVFVVDSSHRDRISEAHSELAKLLTEKE

SEQ ID NO: 165 - protein, 50 aa, Homo sapiens
WDLGGQTSIRPYWRCYYSNTDAVIYVVDSCDRDRIGISKSELVAMLEEEE

SEQ ID NO: 166 - protein, 50 aa, Homo sapiens
WDIGGQESLRSSWNTYYTNTEFVIVVVDSTDRERISVTREELYKMLAHED

SEQ ID NO: 167 - protein, 50 aa, Homo sapiens
WDIGGQESLRSSWNTYYSNTEFIILVVDSIDRERLAITKEELYRMLAHED

SEQ ID NO: 168 - protein, 50 aa, Homo sapiens
WDVGGQKSLRSYWRNYFESTDGLIWVVDSADRQRMQDCQRELQSLLVEER

SEQ ID NO: 169 - protein, 50 aa, Homo sapiens
WDIGGQRKIRPYWKNYFENTDILIYVIDSADRKRFEETGQELAELLEEEK

SEQ ID NO: 170 - protein, 50 aa, Homo sapiens
FDMSGQGRYRNLWEHYYKEGQAIIFVIDSSDRLRMVVAKEELDTLLNHPD

SEQ ID NO: 171 - protein, 50 aa, Homo sapiens
WDVGGQEKLRPLWKSYTRCTDGIVFVVDSVDVERMEEAKTELHKITRISE

SEQ ID NO: 172 - protein, 50 aa, Homo sapiens
WDVGGQEKLRPLWKSYSRCTDGIIYVVDSVDVDRLEEAKTELHKVTKFAE

SEQ ID NO: 173 - protein, 50 aa, Homo sapiens
WDVGGQEKLRPLWRSYTRRTDGLVFVVDAAEAERLEEAKVELHRISRASD

SEQ ID NO: 174 - protein, 50 aa, Homo sapiens
WDVGGQAPLRASWKDYLEGTDILVYVLDSTDEARLPESAAELTEVLNDPN

SEQ ID NO: 175 - protein, 50 aa, Homo sapiens
WDVGGQEKMRTVWGCYCENTDGLVYVVDSTDKQRLEESQRQFEHILKNEH

SEQ ID NO: 176 - protein, 50 aa, Homo sapiens
WDLGGQEELQSLWDKYYAECHGVIYVIDSTDEERLAESKQAFEKVVTSEA

SEQ ID NO: 177 - protein, 50 aa, Homo sapiens
FDLGGGIRIRGIWKNYYAESYGVIFVVDSSDEERMEETKEAMSEMLRHPR

SEQ ID NO: 178 - protein, 50 aa, Homo sapiens
LEIGGSKPFRSYWEMYLSKGLLLIFVVDSADHSRLPEAKKYLHQLIAANP

SEQ ID NO: 179 - protein, 50 aa, Homo sapiens
LEIGGSQNLRFYWKEFVSEVDVLVFVVDSADRLRLPWARQELHKLLDKDP

SEQ ID NO: 180 - protein, 50 aa, Homo sapiens
FDLGGHEQARRVWKNYLPAINGIVFLVDCADHSRLVESKVELNALMTDET

SEQ ID NO: 181 - protein, 50 aa, Homo sapiens
FDLGGHVQARRVWKNYLPAINGIVFLVDCADHERLLESKEELDSLMTDET

SEQ ID NO: 182 - protein, 50 aa, Homo sapiens
WDIGGQPRFRSMWERYCRGVSAIVYMVDAADQEKIEASKNELHNLLDKPQ

SEQ ID NO: 183 - protein, 50 aa, Homo sapiens
WDIGGQPRFRSMWERYCRGVNAIVYMIDAADREKIEASRNELHNLLDKPQ

SEQ ID NO: 184 - protein, 48 aa, Homo sapiens
RELGGCMGPIWSSYYGNCRSLLFVMDASDPTQLSASCVQLLGLLSAEQ

SEQ ID NO: 185 - protein, 50 aa, Homo sapiens
WDTAGQEDYDRLRPLSYPDTDVILMCFSIDSPDSLENIPEKWTPEVKHFC

SEQ ID NO: 186 - protein, 50 aa, Homo sapiens; identical to SEQ ID NO: 185

SEQ ID NO: 187 - protein, 50 aa, Homo sapiens
WDTAGQEDYDRLRPLSYPDTDVILMCFSVDSPDSLENIPEKWVPEVKHFC

SEQ ID NO: 188 - protein, 50 aa, Homo sapiens
WDTSGSSYYDNVRPLAYPDSDAVLICFDISRPETLDSVLKKWQGETQEFC

SEQ ID NO: 189 - protein, 50 aa, Homo sapiens
WDTSGSPYYDNVRPLSYPDSDAVLICFDISRPETLDSVLKKWKGEIQEFC

SEQ ID NO: 190 - protein, 50 aa, Homo sapiens
WDTSGSPYYDNVRPLCYSDSDAVLLCFDISRPETVDSALKKWRTEILDYC

SEQ ID NO: 191 - protein, 50 aa, Homo sapiens
WDTAGQDDYDRLRPLFYPDASVLLLCFDVTSPNSFDNIFNRWYPEVNHFC

SEQ ID NO: 192 - protein, 50 aa, Homo sapiens
YDTAGQEDYDRLRPLSYQNTHLVLICYDVMNPTSYDNVLIKWFPEVTHFC

SEQ ID NO: 193 - protein, 50 aa, Homo sapiens
WDTAGQEDYDRLRPLSYPQTDVFLICFSLVSPASFENVRAKWYPEVRHHC

SEQ ID NO: 194 - protein, 50 aa, Homo sapiens
WDTAGQEDYDRLRPLSYPQTDVFLICFSLVSPASYENVRAKWFPEVRHHC

SEQ ID NO: 195 - protein, 50 aa, Homo sapiens; identical to SEQ ID NO: 193

SEQ ID NO: 196 - protein, 50 aa, Homo sapiens
WDTAGQEEYDRLRTLSYPQTNVFVICFSIASPPSYENVRHKWHPEVCHHC

SEQ ID NO: 197 - protein, 50 aa, Homo sapiens
YDTAGQEDYDRLRPLSYPMTDVFLICFSVVNPASFQNVKEEWVPELKEYA

SEQ ID NO: 198 - protein, 50 aa, Homo sapiens
YDTAGQEDYNQLRPLSYPNTDVFLICFSVVNPASYHNVQEEWVPELKDCM

SEQ ID NO: 199 - protein, 50 aa, Homo sapiens
FDTAGQEDYDRLRPLSYPQTDVFLVCFSVVSPSSFENVKEKWVPEITHHC

SEQ ID NO: 200 - protein, 50 aa, Homo sapiens
WDTAGQEDFDRLRSLCYPDTDVFLACFSVVQPSSFQNITEKWLPEIRTHN

SEQ ID NO: 201 - protein, 50 aa, Homo sapiens
CDTAGQDEFDKLRPLCYTNTDIFLLCFSVVSPSSFQNVSEKWVPEIRCHC

SEQ ID NO: 202 - protein, 50 aa, Homo sapiens
WDTAGNDAFRSIRPLSYQQADVVLMCYSVANHNSFLNLKNKWIGEIRSNL

SEQ ID NO: 203 - protein, 48 aa, Homo sapiens
WDTFGDHHKDRRFAYGRSDVVVLCFSIANPNSLNHVKSMWYPEIKHFC

SEQ ID NO: 204 - protein, 48 aa, Homo sapiens
WDTFGDHHKDRRFAYGRSDVVVLCFSIANPNSLHHVKTMWYPEIKHFC

SEQ ID NO: 205 - protein, 50 aa, Homo sapiens
LDTAGQEEFGAMREQYMRTGEGFLLVFSVTDRGSFEEIYKFQRQILRVKD

SEQ ID NO: 206 - protein, 50 aa, Homo sapiens
LDTAGQEEFSAMREQYMRTGDGFLIVYSVTDKASFEHVDRFHQLILRVKD

SEQ ID NO: 207 - protein, 50 aa, Homo sapiens
LDTAGQAEFTAMRDQYMRAGEGFIICYSITDRRSFHEVREFKQLIYRVRR

SEQ ID NO: 208 - protein, 50 aa, Homo sapiens
LDTAGQAEFTAMREQYMRGGEGFIICYSVTDRQSFQEAAKFKELIFQVRH

SEQ ID NO: 209 - protein, 50 aa, Homo sapiens
LDTAGTEQFTAMRDLYMKNGQGFALVYSITAQSTFNDLQDLREQILRVKD

SEQ ID NO: 210 - protein, 50 aa, Homo sapiens; identical to SEQ ID NO: 209

SEQ ID NO: 211 - protein, 50 aa, Homo sapiens
LDTAGTEQFASMRDLYIKNGQGFILVYSLVNQQSFQDIKPMRDQIIRVKR

SEQ ID NO: 212 - protein, 50 aa, Homo sapiens
LDTAGTEQFASMRDLYIKNGQGFILVYSLVNQQSFQDIKPMRDQIVRVKR

SEQ ID NO: 213 - protein, 50 aa, Homo sapiens; identical to SEQ ID NO: 211

SEQ ID NO: 214 - protein, 50 aa, Homo sapiens
LDTAGQEDYAAIRDNYFRSGEGFLCVFSITEMESFAATADFREQILRVKE

SEQ ID NO: 215 - protein, 50 aa, Homo sapiens
LDTAGQEDYAAIRDNYFRSGEGFLLVFSITEHESFTATAEFREQILRVKA

SEQ ID NO: 216 - protein, 46 aa, Homo sapiens
LDTAGQAIHRALRDQCLAVCDGVLGVFALDDPSSLIQLQQIWATWG

SEQ ID NO: 217 - protein, 51 aa, Homo sapiens
LDMWENKGENEWLHDHCMQVGDAYLIVYSITDRASFEKASELRIQLRRARQ

SEQ ID NO: 218 - protein, 50 aa, Homo sapiens
YDIWEQDGGRWLPGHCMAMGDAYVIVYSVTDKGSFEKASELRVQLRRARQ

SEQ ID NO: 219 - protein, 52 aa, Homo sapiens
VDTWEAEKLDKSWSQESCLQGGSAYVIVYSIADRGSFESASELRIQLRRTHQ

SEQ ID NO: 220 - protein, 51 aa, Homo sapiens
YDIWEQGDAGGWLRDHCLQTGDAFLIVFSVTDRRSFSKVPETLLRLRAGRP

SEQ ID NO: 221 - protein, 49 aa, Homo sapiens
LDTAGQEDTIQREGHMRWGEGFVLVYDITDRGSFEEVLPLKNILDEIKK

SEQ ID NO: 222 - protein, 49 aa, Homo sapiens
MDTADLDTPRNCERYLNWAHAFLVVYSVDSRQSFDSSSSYLELLALHAK

SEQ ID NO: 223 - protein, 57 aa, Homo sapiens
QDTPGGVQIQDSLPQVVDSLSKCVQWAEGFLLVYSITDYDSYLSIRPLYQHIRKVHP

SEQ ID NO: 224 - protein, 56 aa, Homo sapiens
QDTPGIQVHENSLSCSEQLNRCIRWADAVVIVFSITDYKSYELISQLHQHVQQLHL

SEQ ID NO: 225 - protein, 51 aa, Homo sapiens
YDPCSQTQKAKFSLTSELHWADGFVIVYDISDRSSFAFAKALIYRIREPQT

SEQ ID NO: 226 - protein, 50 aa, Homo sapiens
TDTTGSHQFPAMQRLSISKGHAFILVFSVTSKQSLEELGPIYKLIVQIKG

SEQ ID NO: 227 - protein, 50 aa, Homo sapiens
TDTTGSHQFPAMQRLSISKGHAFILVYSITSRQSLEELKPIYEQICEIKG

SEQ ID NO: 228 - protein, 50 aa, Homo sapiens
TDSKSGDGNRALQRHVIARGHAFVLVYSVTKKETLEELKAFYELICKIKG

SEQ ID NO: 229 - protein, 50 aa, Homo sapiens
LDTSGNHPFPAMRRLSILTGDVFILVFSLDNRDSFEEVQRLRQQILDTKS

SEQ ID NO: 230 - protein, 50 aa, Homo sapiens
LDTSGNHPFPAMRRLSILTGDVFILVFSLDNRESFDEVKRLQKQILEVKS

SEQ ID NO: 231 - protein, 58 aa, Homo sapiens
RDGDVAGPGSSPGGPEEWPDAKDWSLQDTDAFVLVYDICSPDSFDYVKALRQRIAETR

SEQ ID NO: 232 - protein, 57 aa, Homo sapiens
LDFPPISAFPVNTLQEWADTCCRGLRSVHAYILVYDICCFDSFEYVKTIRQQILETR

SEQ ID NO: 233 - protein, 50 aa, Homo sapiens
VDTAGQDEYSIFPQTYSIDINGYILVYSVTSIKSFEVIKVIHGKLLDMVG

SEQ ID NO: 234 - protein, 50 aa, Homo sapiens
VDTAGQDEYSILPYSFIIGVHGYVLVYSVTSLHSFQVIESLYQKLHEGHG

SEQ ID NO: 235 - protein, 50 aa, Homo sapiens
YDTRGLQEGVELPKHYFSFADGFVLVYSVNNLESFQRVELLKKEIDKFKD

SEQ ID NO: 236 - protein, 50 aa, Homo sapiens
YDTRGLRDGAELPRHCFSCTDGYVLVYSTDSRESFQRVELLKKEIDKSKD

SEQ ID NO: 237 - protein, 50 aa, Homo sapiens
WDTSGQGRFCTIFRSYSRGAQGVILVYDIANRWSFEGMDRWIKKIEEHAP

SEQ ID NO: 238 - protein, 50 aa, Homo sapiens
WDTSGQGRFCTIFRSYSRGAQGVILVYDIANRWSFDGIDRWIKEIDEHAP

SEQ ID NO: 239 - protein, 50 aa, Homo sapiens
WDTSGQGRFCTIFRSYSRGAQGILLVYDITNRWSFDGIDRWIKEIDEHAP

SEQ ID NO: 240 - protein, 49 aa, Homo sapiens
WDTAGQERFRTITSSYYRGAHGIIVVYDVTDQESFNNVKQWLQEIDRYA

SEQ ID NO: 241 - protein, 49 aa, Homo sapiens
WDTAGQERFRTITSSYYRGAHGIIVVYDVTDQESYANVKQWLQEIDRYA

SEQ ID NO: 242 - protein, 48 aa, Homo sapiens
WDTAGQERFRTITSTYYRGTHGVIVVYDVTSAESFVNVKRWLHEINQN

SEQ ID NO: 243 - protein, 49 aa, Homo sapiens
WDTAGQERYRTITTAYYRGAMGFILMYDITNEESFNAVQDWSTQIKTYS

SEQ ID NO: 244 - protein, 49 aa, Homo sapiens; identical to SEQ ID NO: 243

SEQ ID NO: 245 - protein, 49 aa, Homo sapiens
WDTAGQERYRTITTAYYRGAMGFILMYDITNEESFNAVQDWATQIKTYS

SEQ ID NO: 246 - protein, 49 aa, Homo sapiens
WDTAGQERYRTITTAYYRGAMGFLLMYDIANQESFAAVQDWATQIKTYS

SEQ ID NO: 247 - protein, 49 aa, Homo sapiens
WDTAGQERFRTITTAYYRGAMGIMLVYDITNEKSFDNIRNWIRNIEEHA

SEQ ID NO: 248 - protein, 49 aa, Homo sapiens
WDTAGQERFRTITTAYYRGAMGIMLVYDITNEKSFDNIKNWIRNIEEHA

SEQ ID NO: 249 - protein, 49 aa, Homo sapiens
WDTAGQERFHTITTSYYRGAMGIMLVYDITNGKSFENISKWLRNIDEHA

SEQ ID NO: 250 - protein, 49 aa, Homo sapiens
WDTAGQERFKTITTAYYRGAMGIILVYDITDEKSFENIQNWMKSIKENA

SEQ ID NO: 251 - protein, 49 aa, Homo sapiens
WDTAGQERFNSITSAYYRSAKGIILVYDITKKETFDDLPKWMKMIDKYA

SEQ ID NO: 252 - protein, 50 aa, Homo sapiens
WDTAGQERFRTLTPSYYRGAQGVILVYDVTRRDTFVKLDNWLNELETYCT

SEQ ID NO: 253 - protein, 50 aa, Homo sapiens
WDTAGQERFRTITQSYYRSAHAAIIAYDLTRRSTFESIPHWIHEIEKYGA

SEQ ID NO: 254 - protein, 50 aa, Homo sapiens
WDTAGQERFRTITQSYYRSANGAILAYDITKRSSFLSVPHWIEDVRKYAG

SEQ ID NO: 255 - protein, 50 aa, Homo sapiens
WDTAGQERFRSITQSYYRSANALILTYDITCEESFRCLPEWLREIEQYAS

SEQ ID NO: 256 - protein, 51 aa, Homo sapiens
WDTAGQERFRKSMVEHYYRNVHAVVFVYDVTKMTSFTNLKMWIQECNGHAV

SEQ ID NO: 257 - protein, 51 aa, Homo sapiens
WDTAGQERFRKSMVQHYYRNVHAVVFVYDMTNMASFHSLPSWIEECKQHLL

SEQ ID NO: 258 - protein, 50 aa, Homo sapiens
WDTAGQESFRSITRSYYRGAAGALLVYDITRRDTFNHLTTWLEDARQHSN

SEQ ID NO: 259 - protein, 50 aa, Homo sapiens
WDTAGQESFRSITRSYYRGAAGALLVYDITRRETFNHLTSWLEDARQHSS

SEQ ID NO: 260 - protein, 50 aa, Homo sapiens
WDTAGQERFRSVTRSYYRGAAGALLVYDITSRETYNALTNWLTDARMLAS

SEQ ID NO: 261 - protein, 50 aa, Homo sapiens
WDTAGQERFRSVTRSYYRGAAGALLVYDITSRETYNSLAAWLTDARTLAS

SEQ ID NO: 262 - protein, 50 aa, Homo sapiens
WDTAGQERFRAVTRSYYRGAAGALMVYDITRRSTYNHLSSWLTDARNLTN

SEQ ID NO: 263 - protein, 50 aa, Homo sapiens
WDTAGQERYRAITSAYYRGAVGALLVYDIAKHLTYENVERWLKELRDHAD

SEQ ID NO: 264 - protein, 50 aa, Homo sapiens
WDTAGQERYRRITSAYYRGAVGALLVYDIAKHLTYENVERWLKELRDHAD

SEQ ID NO: 265 - protein, 50 aa, Homo sapiens
WDTAGLERYRAITSAYYRGAVGALLVFDLTKHQTYAVVERWLKELYDHAE

SEQ ID NO: 266 - protein, 50 aa, Homo sapiens
WDTAGQERFRSITRSYYRNSVGGFLVFDITNRRSFEHVKDWLEEAKMYVQ

SEQ ID NO: 267 - protein, 50 aa, Homo sapiens
WDTAGQERFRSITRAYYRNSVGGLLLFDITNRRSFQNVHEWLEETKVHVQ

SEQ ID NO: 268 - protein, 50 aa, Homo sapiens
WDTAGHERFRCITRSFYRNVVGVLLVFDVTNRRSFEHIQDWHQEVMATQG

SEQ ID NO: 269 - protein, 50 aa, Homo sapiens
WDTAGQERFRSVTHAYYRDAHALLLLYDVTNKASFDNIQAWLTEIHEYAQ

SEQ ID NO: 270 - protein, 50 aa, Homo sapiens
WDTAGQERFRSVTHAYYRDAQALLLLYDITNKSSFDNIRAWLTEIHEYAQ

SEQ ID NO: 271 - protein, 50 aa, Homo sapiens
WDTAGQERFRSIAKSYFRKADGVLLLYDVTCEKSFLNIREWVDMIEDAAH

SEQ ID NO: 272 - protein, 50 aa, Homo sapiens
WDTAGQERFRSLTTAFFRDAMGFLLLFDLTNEQSFLNVRNWISQLQMHAY

SEQ ID NO: 273 - protein, 50 aa, Homo sapiens
WDTAGQERFRSLTTAFFRDAMGFLLMFDLTSQQSFLNVRNWMSQLQANAY

SEQ ID NO: 274 - protein, 50 aa, Homo sapiens
WDTAGQEEFDAITKAYYRGAQACVLVFSTTDRESFEAVSSWREKVVAEVG

SEQ ID NO: 275 - protein, 50 aa, Homo sapiens
FDSAGKELFSEMLDKLWESPNVLCLVYDVTNEESFNNCSKWLEKARSQAP

SEQ ID NO: 276 - protein, 50 aa, Homo sapiens
WDTAGQERFRSLRTPFYRGSDCCLLTFSVDDSQSFQNLSNWKKEFIYYAD

SEQ ID NO: 277 - protein, 50 aa, Homo sapiens
WDTAGQERFKSLRTPFYRGADCCLLTFSVDDRQSFENLGNWQKEFIYYAD

SEQ ID NO: 278 - protein, 50 aa, Homo sapiens
WDTAGQERFQSLGVAFYRGADCCVLVFDVTAPNTFKTLDSWRDEFLIQAS

SEQ ID NO: 279 - protein, 50 aa, Homo sapiens
WDTGGQERFRSMVSTFYKGSDGCILAFDVTDLESFEALDIWRGDVLAKIV

SEQ ID NO: 280 - protein, 50 aa, Homo sapiens
WDIAGQERFGNMTRVYYKEAVGAFVVFDISRSSTFEAVLKWKSDLDSKVH

SEQ ID NO: 281 - protein, 50 aa, Homo sapiens
WDIAGQERFGNMTRVYYREAMGAFIVFDVTRPATFEAVAKWKNDLDSKLS

SEQ ID NO: 282 - protein, 50 aa, Homo sapiens
WDIAGQERFTSMTRLYYRDASACVIMFDVTNATTFSNSQRWKQDLDSKLT

SEQ ID NO: 283 - protein, 47 aa, Homo sapiens
WDTAGQERYHSLAPMYYRGAQAAIVVYDITNEESFARAKNWVKELQR

SEQ ID NO: 284 - protein, 47 aa, Homo sapiens
WDTAGQERYHSLAPMYYRGAQAAIVVYDITNTDTFARAKNWVKELQR

SEQ ID NO: 285 - protein, 47 aa, Homo sapiens
WDTAGQERYHSLAPMYYRGAQAAIVVYDITNQETFARAKTWVKELQR

SEQ ID NO: 286 - protein, 47 aa, Homo sapiens
WDTAGQERFRALAPMYYRGSAAAIIVYDITKEETFSTLKNWVKELRQ

SEQ ID NO: 287 - protein, 47 aa, Homo sapiens
WDTAGQERFHSLAPMYYRGSAAAVIVYDITKQDSFYTLKKWVKELKE

SEQ ID NO: 288 - protein, 47 aa, Homo sapiens
WDTAGSERYEAMSRIYYRGAKAAIVCYDLTDSSSFERAKFWVKELRS

SEQ ID NO: 289 - protein, 47 aa, Homo sapiens
WDTAGQERFHALGPIYYRDSNGAILVYDITDEDSFQKVKNWVKELRK

SEQ ID NO: 290 - protein, 48 aa, Homo sapiens
WDTAGQEKYHSVCHLYFRGANAALLVYDITRKDSFLKAQQWLKDLEEE

SEQ ID NO: 291 - protein, 47 aa, Homo sapiens
WDTAGQERFRSLIPSYIRDSAAAVVVYDITNVNSFQQTTKWIDDVRT

SEQ ID NO: 292 - protein, 47 aa, Homo sapiens
WDTAGQERLRSLIPRYIRDSAAAVVVYDITNVNSFQQTTKWIDDVRT

SEQ ID NO: 293 - protein, 47 aa, Homo sapiens
WDTAGQERFRSLIPSYIRDSTVAVVVYDITNLNSFQQTSKWIDDVRT

SEQ ID NO: 294 - protein, 50 aa, Homo sapiens
WDIGGQTIGGKMLDKYIYGAQGVLLVYDITNYQSFENLEDWYTVVKKVSE

SEQ ID NO: 295 - protein, 50 aa, Homo sapiens
WDTAGQERYQTITKQYYRRAQGIFLVYDISSERSYQHIMKWVSDVDEVGD

SEQ ID NO: 296 - protein, 50 aa, Homo sapiens
WDTAGQERFKCIASTYYRGAQAIIIVFNLNDVASLEHTKQWLADALKEND

SEQ ID NO: 297 - protein, 50 aa, Homo sapiens
WDTAGQEKFKCIASAYYRGAQVIITAFDLTDVQTLEHTRQWLEDALRENE

SEQ ID NO: 298 - protein, 50 aa, Homo sapiens
WDTAGQEKFGGLRDGYYIQAQCAIIMFDVTSRVTYKNVPNWHRDLVRVCE

SEQ ID NO: 299 - protein, 49 aa, Homo sapiens
WDTAGQERFQSMHASYYHKAHACIMVFDIQRKVTYRNLSTWYTELREFR

SEQ ID NO: 300 - protein, 49 aa, Homo sapiens
WDTAGQERFQSMHASYYHKAHACIMVFDVQRKVTYRNLSTWYTELREFR

SEQ ID NO: 301 - protein, 50 aa, Homo sapiens
VDYSEAEQSDEQLHQEISQANVICIVYAVNNKHSIDKVTSRWIPLINERT

SEQ ID NO: 302 - protein, 50 aa, Homo sapiens
VDYSEAEQTDEELREEIHKANVVCVVYDVSEEATIEKIRTKWIPLVNGGT

SEQ ID NO: 303 - protein, 50 aa, Homo sapiens
WDTAGREQFHGLGSMYCRGAAAIILTYDVNHRQSLVELEDRFLGLTDTAS

SEQ ID NO: 304 - protein, 54 aa, Homo sapiens
WDVGGSVGSASSVKSTRAVFYNSVNGIIFVHDLTNKKSSQNLRRWSLEALNRDL

SEQ ID NO: 305 - protein, 51 aa, Homo sapiens
KELGGADNIRKYWSRYYQGSQGVIFVLDSASSEDDLEAARNELHSALQHPQ

SEQ ID NO: 306 - protein, 50 aa, Homo sapiens
WDCGGDAKFESCWPALMKDAHGVVIVFNADIPSHRKEMEMWYSCFVQQPS

SEQ ID NO: 307 - protein, 52 aa, Homo sapiens
IDLPGHESLRLQFLERFKSSAGAIVFVVDSAAFQREVKDVAEFLYQVLIDSM

SEQ ID NO: 308 - protein, 51 aa, Homo sapiens
GAELGAPGRAEVGRTDAERKGSRIPRGAPPEASRAAAAGAPALSPPRALPS

(19) United States
(12) Patent Application Publication
(10) Pub. No.: US 2017/0158702 A1
(43) Pub. Date: Jun. 8, 2017

(54) MULTIVALENT RAS BINDING COMPOUNDS

(71) Applicant: KYRAS THERAPEUTICS, INC., New York, NY (US)

(72) Inventors: Joseph P. VACCA, Telford, PA (US); Dansu LI, Warrington, PA (US)

(21) Appl. No.: 15/366,637

(22) Filed: Dec. 1, 2016

Related U.S. Application Data
(60) Provisional application No. 62/262,290, filed on Dec. 2, 2015.
(60) Provisional application No. 62/262,295, filed on Dec. 2, 2015.
(60) Provisional application No. 62/363,140, filed on Jul. 15, 2016.

(51) Int. Cl. C07D 487/08; C07D 231/56; C07D 209/14; C07D 401/12; C07D 403/12; C07D 403/04; C07D 487/04; C07D 471/08; C07D 401/14; C07D 405/12; C07D 401/08; C07D 401/04; C07D 471/04; C07D 471/10

(52) U.S. Cl. CPC C07D 487/08; C07D 471/04; C07D 231/56; C07D 209/14; C07D 401/12; C07D 403/12; C07D 471/10; C07D 487/04; C07D 471/08; C07D 401/14; C07D 405/12; C07D 401/08; C07D 401/04; C07D 403/04

(57) ABSTRACT

Described herein are compounds that modulate Ras signaling, methods of making such compounds, pharmaceutical compositions and medicaments comprising such compounds, and methods of using such compounds in the treatment of conditions, diseases, or disorders associated with altered Ras signaling. Further described herein are compounds, methods of making such compounds, pharmaceutical compositions and medicaments comprising such compounds and methods of using such compounds in the treatment of cell proliferative disorders, including cancer.

### CROSS-REFERENCE

This application claims benefit of U.S. Provisional Patent Application No. 62/262,290 filed on Dec. 2, 2015, U.S. Provisional Patent Application No. 62/262,295 filed on Dec. 2, 2015, and U.S. Provisional Patent Application No. 62/363,140 filed on Jul. 15, 2016, each incoporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 28, 2016, is named 49315-702_201_SL.TXT and is 139,916 bytes in size.

### BACKGROUND

Ras GTPases form a large family of proteins with many members confirmed as targets in cancer. Ras gene mutations are found at high rates in three of the top four lethal malignancies in the United States—pancreatic (90%), colon (45%), and lung cancers (35%). In addition, many tumors have been shown to be dependent on continued expression of oncogenic Ras proteins in cell and animal models. On a cellular level, the Ras proteins play a central role in a number of signal transduction pathways controlling cell growth and differentiation. However, Ras proteins have been viewed as challenging targets, primarily due to the lack of a sufficiently large and deep hydrophobic site for small molecule binding, aside from the GTP-binding site. For these reasons, traditional high-throughput screening has been unable to provide high affinity small molecule Ras ligands. Thus, there exists an unmet need for compounds that selectively bind a Ras protein.

### BRIEF SUMMARY

In one aspect, provided herein is a compound of Formula (Ia), or a pharmaceutically acceptable salt, or solvate thereof:

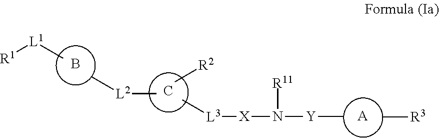

wherein,

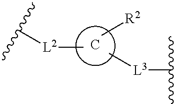

is a bicyclic heteroaryl that is selected from the following structures:

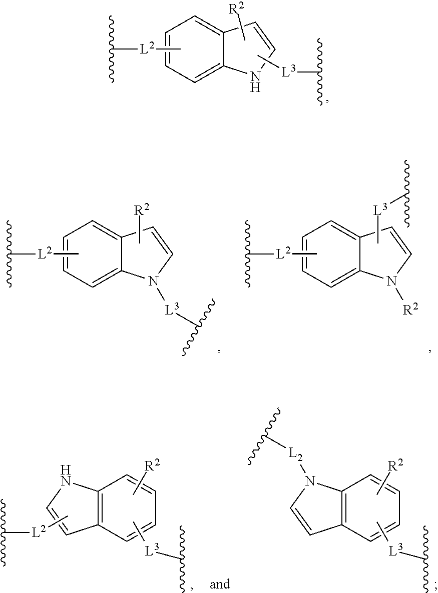

L¹ and L² are each independently an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N with the proviso that Ring B is not:

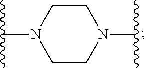 [SMILES: *N1CCN(*)CC1 ; ";"]

wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)— or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N;
wherein if Ring A is substituted, then Ring A is substituted with at least one R^{A}; each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring, and
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring,
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl).

In some embodiments,

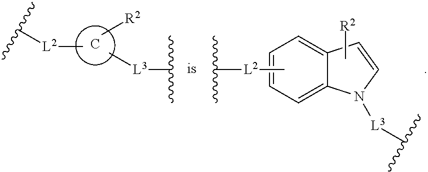

In some embodiments,

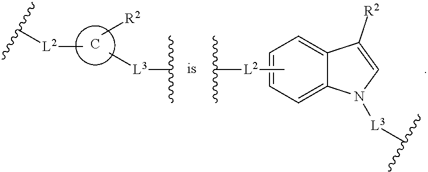

In some embodiments, a compound of Formula (Ia) has the following structure of Formula (Ib), or a pharmaceutically acceptable salt, or solvate thereof:

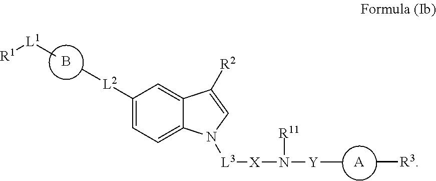

In some embodiments, a compound of Formula (Ia) has the following structure of Formula (Ic), or a pharmaceutically acceptable salt, or solvate thereof:

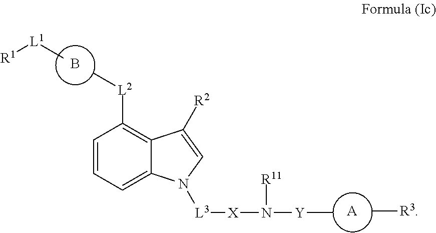

In some embodiments,

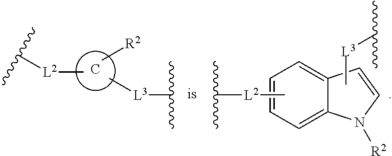

In some embodiments,

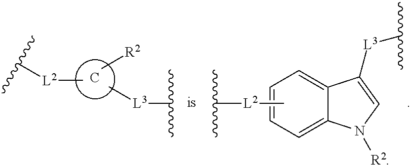

In some embodiments, a compound of Formula (Ia) has the following structure of Formula (Id), or a pharmaceutically acceptable salt, or solvate thereof:

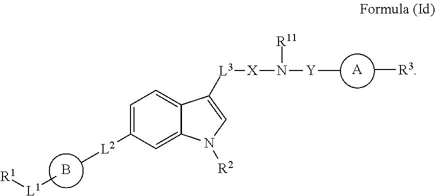

In some embodiments, a compound of Formula (Ia) has the following structure of Formula (Ie), or a pharmaceutically acceptable salt, or solvate thereof:

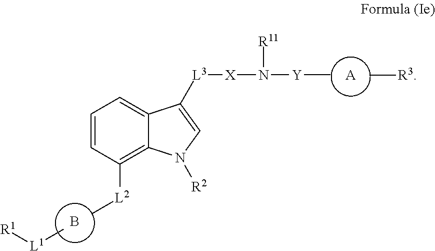

In some embodiments,

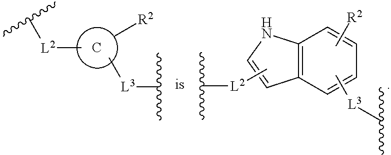

In some embodiments,

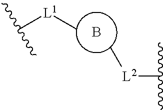

is selected from the following:

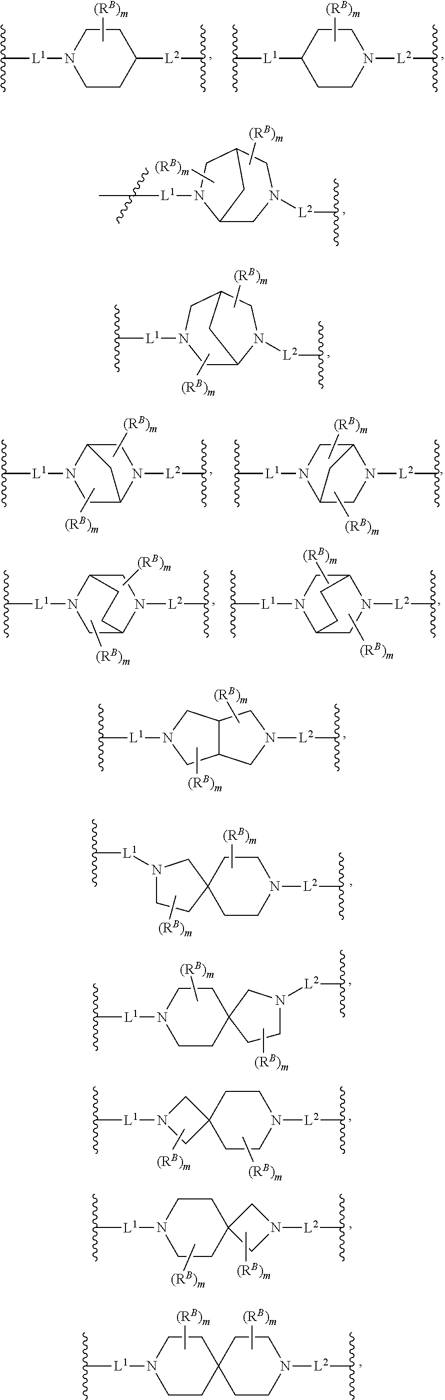

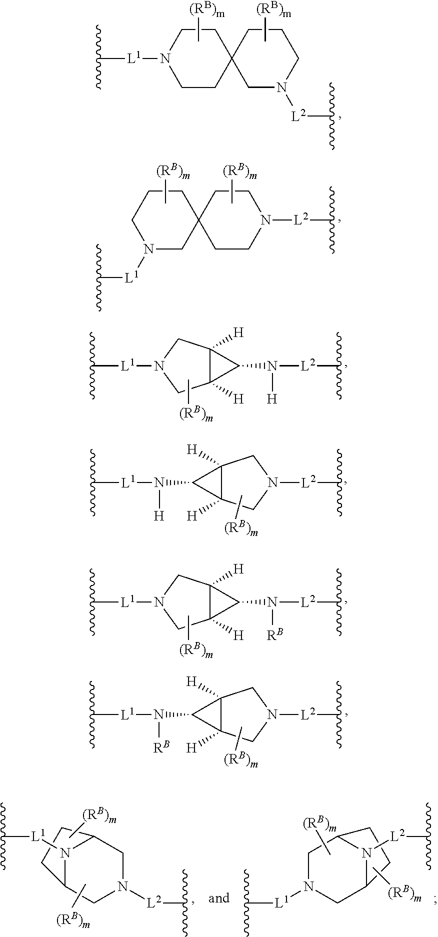

and each m is independently 0, 1, 2, 3, or 4.

In some embodiments,

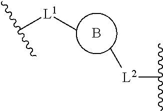

is selected from the following:

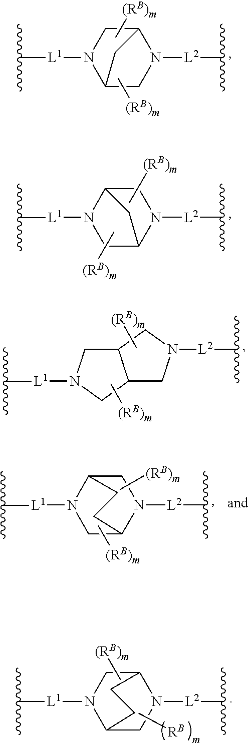

In some embodiments,

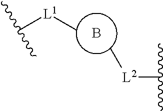

is selected from the following:

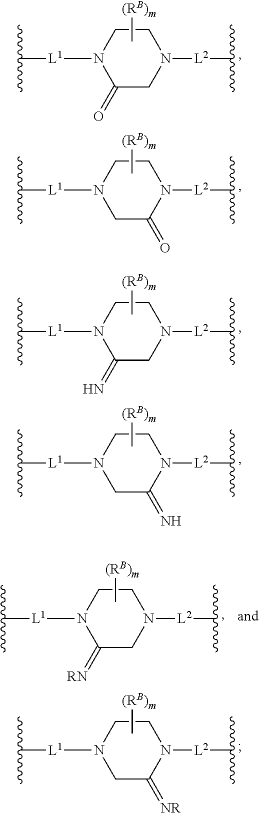

and

R is optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R is C₁-C₄alkyl.

In some embodiments,

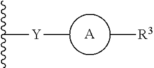

is selected from the following:

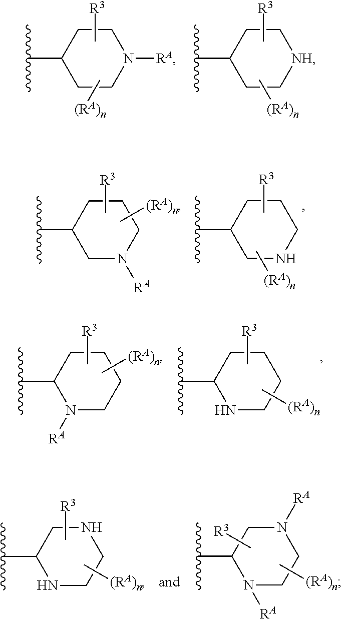

and each n is independently 0, 1, 2, 3, or 4.

In some embodiments,

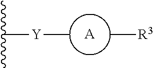

is selected from the following:

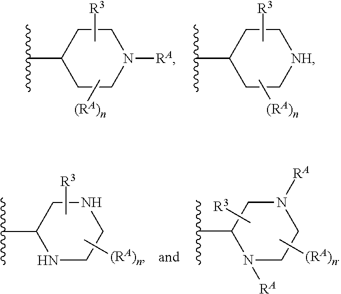

In some embodiments,

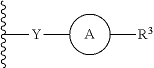

is selected from the following:

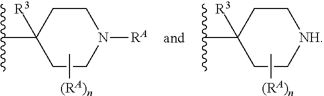

In some embodiments, L¹ is —CH₂—, C(═O), O, S, S(═O), S(═O)₂, or NR⁴. In some embodiments, L¹ is —CH_{2—}. In some embodiments, L² is —CH₂—, C(═O), O, S, S(═O), S(═O)₂, or NR⁴. In some embodiments, L² is —CH₂—. In some embodiments, L³ is absent, —CH₂—, —CH₂—CH₂—, or —CH₂—CH₂—CH₂—. In some embodiments, L³ is —CH₂—CH₂—.

In some embodiments, L³ is:

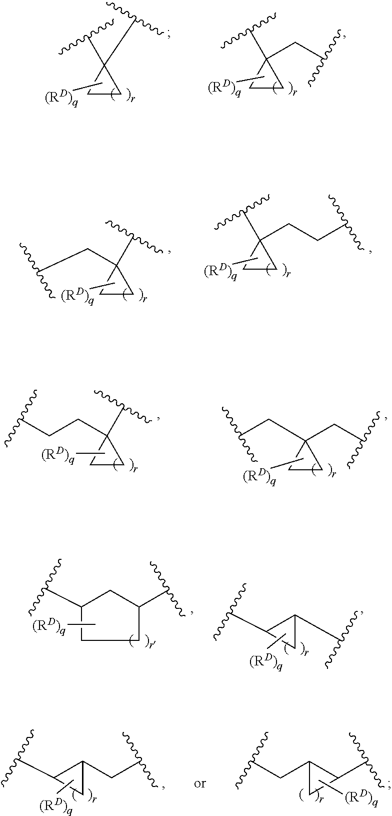

each q is independently 0, 1, 2, 3, or 4;

r is 1, 2, 3, 4, or 5; and

r′ is 1 or 2.

In some embodiments, X is —CH₂— or C(═O). In some embodiments, X is —CH₂—.

In some embodiments, X is:

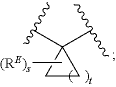

each s is independently 0, 1, 2, 3, or 4; and

t is 1, 2, 3, 4, or 5.

In some embodiments, X is:

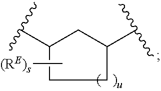

each s is independently 0, 1, 2, 3, or 4; and

u is 0, 1, or 2.

In some embodiments, L³-X is —CH₂—CH₂—CH₂—. In some embodiments, Y is —CH₂— or C(═O). In some embodiments, Y is C(═O). In some embodiments, R¹¹ is hydrogen.

In some embodiments, R³ is H. In some embodiments, R³ is CH₂N(R⁹)(R¹⁰). In some embodiments, R³ is N(R⁹)(R¹⁰).

In some embodiments, R⁹ and R¹⁰ are each H. In some embodiments, R⁹ is H and R¹⁰ is —C₁-C₄alkylene-(optionally substituted phenyl) or —C₁-C₄alkylene-(optionally substituted heteroaryl). In some embodiments, R⁹ is H and R¹⁰ is —CH₂-(optionally substituted phenyl). In some embodiments, R⁹ is H and R¹⁰ is —CH₂-(optionally substituted heteroaryl).

In some embodiments, R³ is CH₂N(R⁹)(R¹⁰); and R⁹ and R¹⁰ are each H. In some embodiments, R³ is CH₂N(R⁹)(R¹⁰); and R⁹ is H and R¹⁰ is —C₁-C₄alkylene-(optionally substituted phenyl) or —C₁-C₄alkylene-(optionally substituted heteroaryl). In some embodiments, R³ is CH₂N(R⁹)(R¹⁰); and R⁹ is H and R¹⁰ is —C₁-C₄alkylene-(optionally substituted phenyl). In some embodiments, R³ is CH₂N(R⁹)(R¹⁰); and R⁹ is H and R¹⁰ is —C₁-C₄alkylene-(optionally substituted heteroaryl). In some embodiments, R³ is N(R⁹)(R¹⁰); and R⁹ and R¹⁰ are each H. In some embodiments, R³ is N(R⁹)(R¹⁰); and R⁹ is H and R¹⁰ is —C₁-C₄alkylene-(optionally substituted phenyl) or —C₁-C₄alkylene-(optionally substituted heteroaryl). In some embodiments, R³ is N(R⁹)(R¹⁰); and R⁹ is H and R¹⁰ is —C₁-C₄alkylene-(optionally substituted phenyl). In some embodiments, R³ is N(R⁹)(R¹⁰); and R⁹ is H and R¹⁰ is —C₁-C₄alkylene-(optionally substituted heteroaryl).

In some embodiments, R¹ is an unsubstituted phenyl. In some embodiments, R¹ is a substituted phenyl. In some embodiments, R¹ is selected from:

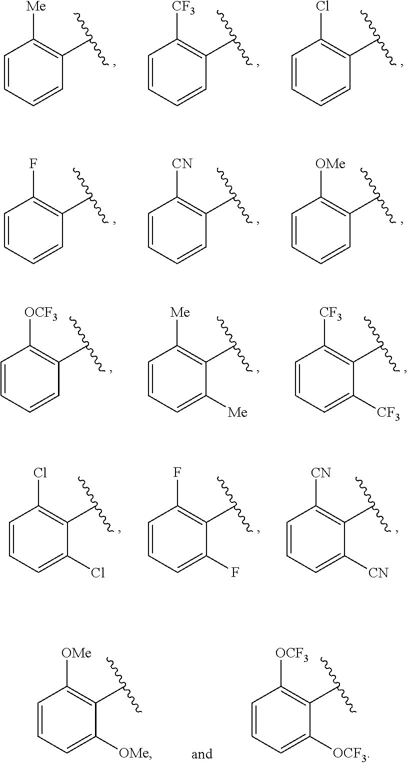

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

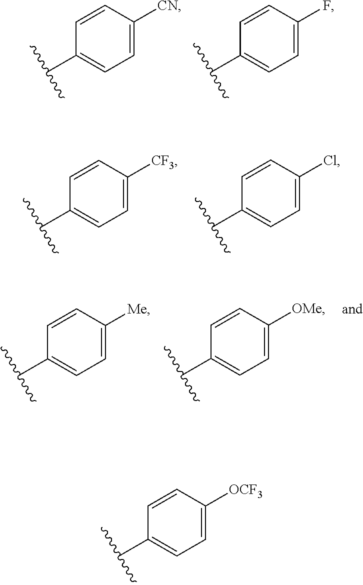

In some embodiments, R² is selected from:

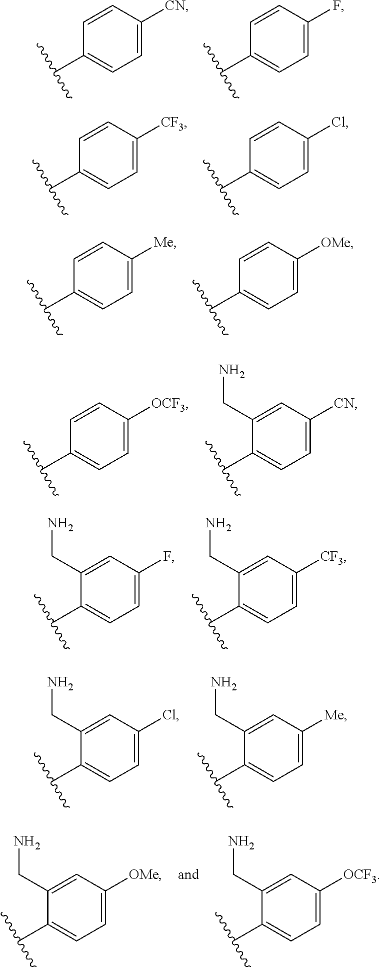

In some embodiments, R¹ is optionally substituted heterocycloalkyl. In some embodiments, R¹ is selected from:

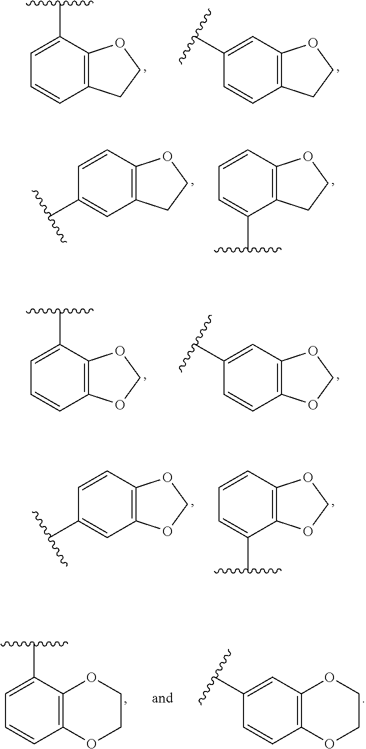

In some embodiments, R² is optionally substituted heterocycloalkyl. In some embodiments, R² is selected from:

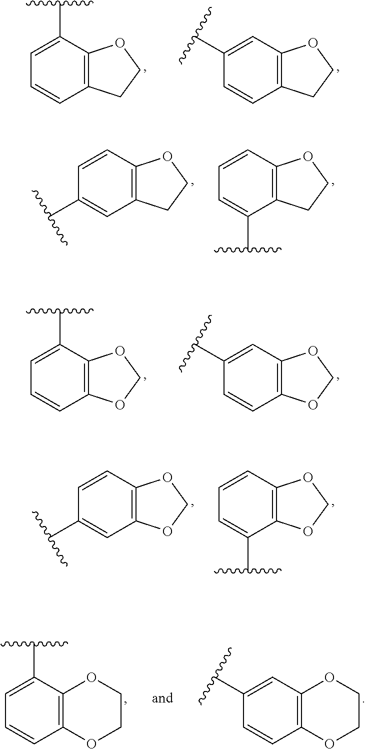

In some embodiments, a compound of Formula (Ia) is selected from:

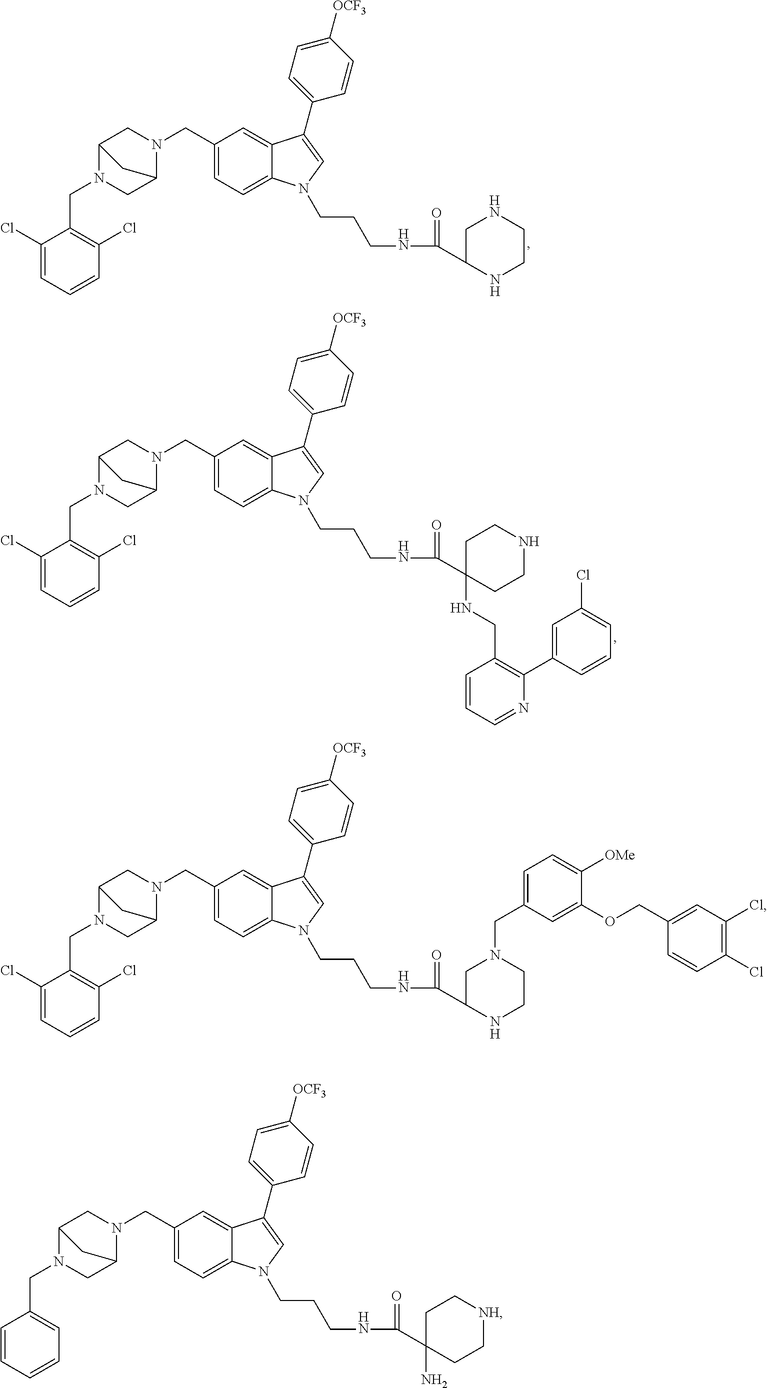 [SMILES: O=C(NCCCn1cc(-c2ccc(OC(F)(F)F)cc2)c2cc(CN3CC4CC3CN4Cc3c(Cl)cccc3Cl)ccc21)C1CNCCN1 ; O=C(NCCCn1cc(-c2ccc(OC(F)(F)F)cc2)c2cc(CN3CC4CC3CN4Cc3c(Cl)cccc3Cl)ccc21)C1(NCc2cccnc2-c2cccc(Cl)c2)CCNCC1 ; COc1ccc(CN2CCNC(C(=O)NCCCn3cc(-c4ccc(OC(F)(F)F)cc4)c4cc(CN5CC6CC5CN6Cc5c(Cl)cccc5Cl)ccc43)C2)cc1OCc1ccc(Cl)c(Cl)c1 ; NC1(C(=O)NCCCn2cc(-c3ccc(OC(F)(F)F)cc3)c3cc(CN4CC5CC4CN5Cc4ccccc4)ccc32)CCNCC1]

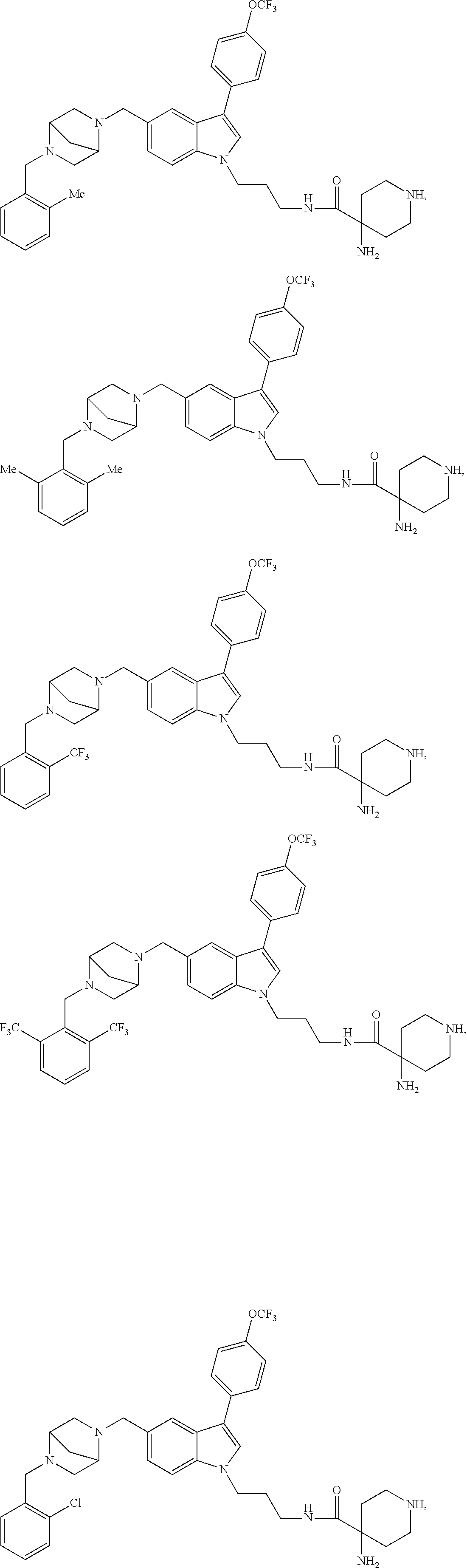 [SMILES: Cc1ccccc1CN1CC2CC1CN2Cc1ccc2c(c1)c(-c1ccc(OC(F)(F)F)cc1)cn2CCCNC(=O)C1(N)CCNCC1 ; Cc1cccc(C)c1CN1CC2CC1CN2Cc1ccc2c(c1)c(-c1ccc(OC(F)(F)F)cc1)cn2CCCNC(=O)C1(N)CCNCC1 ; NC1(C(=O)NCCCn2cc(-c3ccc(OC(F)(F)F)cc3)c3cc(CN4CC5CC4CN5Cc4ccccc4C(F)(F)F)ccc32)CCNCC1 ; NC1(C(=O)NCCCn2cc(-c3ccc(OC(F)(F)F)cc3)c3cc(CN4CC5CC4CN5Cc4c(C(F)(F)F)cccc4C(F)(F)F)ccc32)CCNCC1 ; NC1(C(=O)NCCCn2cc(-c3ccc(OC(F)(F)F)cc3)c3cc(CN4CC5CC4CN5Cc4ccccc4Cl)ccc32)CCNCC1]

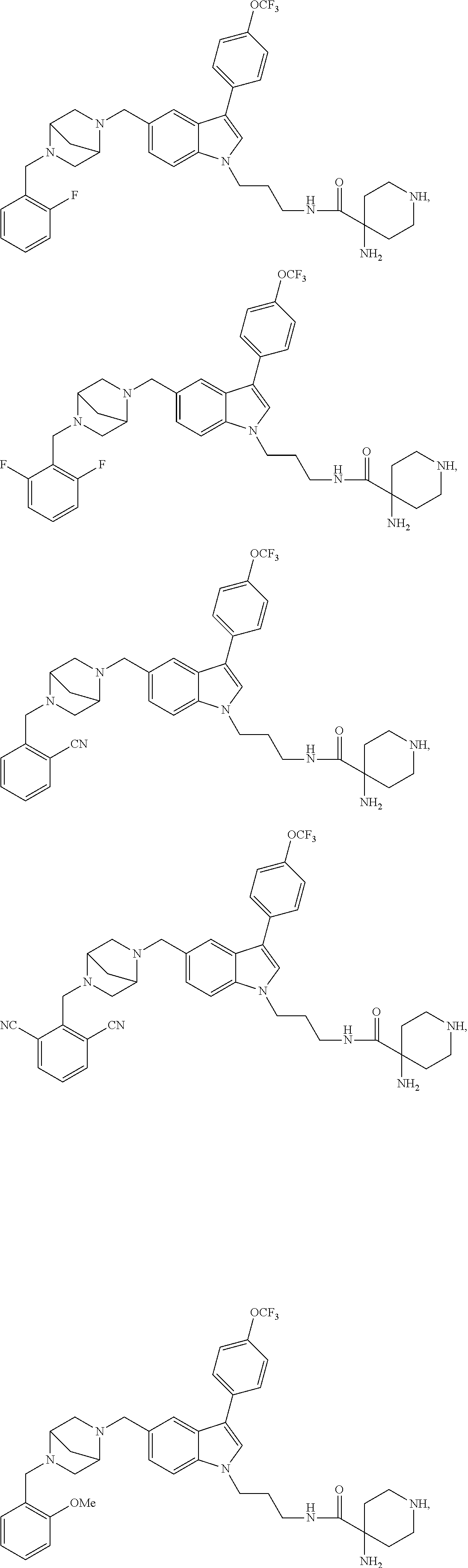 [SMILES: NC1(C(=O)NCCCn2cc(-c3ccc(OC(F)(F)F)cc3)c3cc(CN4CC5CC4CN5Cc4ccccc4F)ccc32)CCNCC1 ; NC1(C(=O)NCCCn2cc(-c3ccc(OC(F)(F)F)cc3)c3cc(CN4CC5CC4CN5Cc4c(F)cccc4F)ccc32)CCNCC1 ; N#Cc1ccccc1CN1CC2CC1CN2Cc1ccc2c(c1)c(-c1ccc(OC(F)(F)F)cc1)cn2CCCNC(=O)C1(N)CCNCC1 ; N#Cc1cccc(C#N)c1CN1CC2CC1CN2Cc1ccc2c(c1)c(-c1ccc(OC(F)(F)F)cc1)cn2CCCNC(=O)C1(N)CCNCC1 ; COc1ccccc1CN1CC2CC1CN2Cc1ccc2c(c1)c(-c1ccc(OC(F)(F)F)cc1)cn2CCCNC(=O)C1(N)CCNCC1]

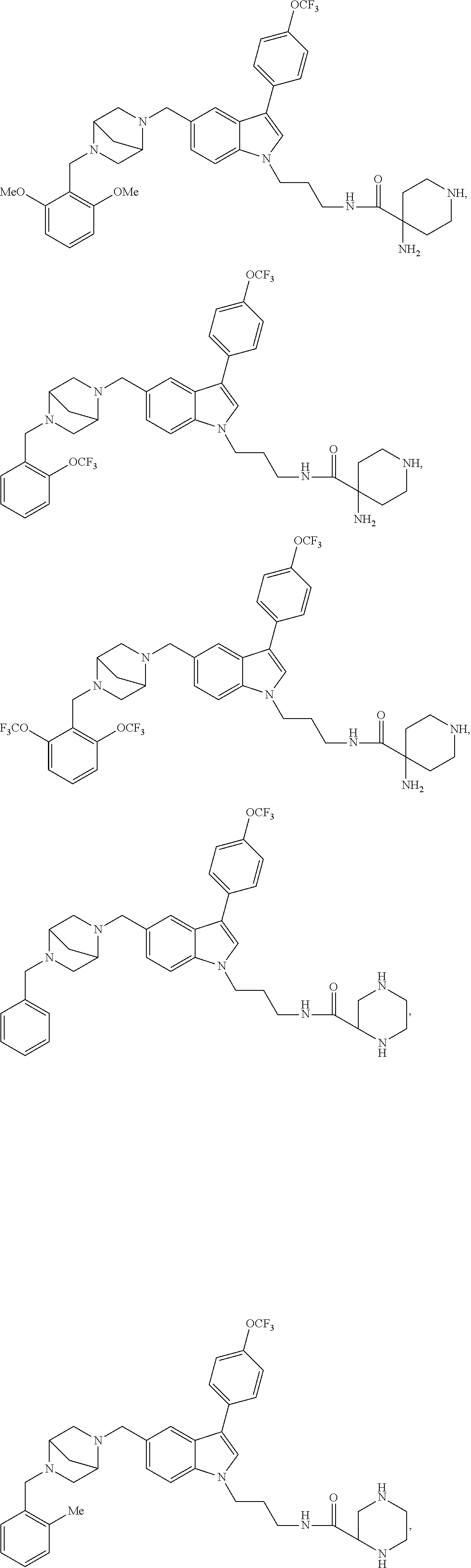 [SMILES: COc1cccc(OC)c1CN1CC2CC1CN2Cc1ccc2c(c1)c(-c1ccc(OC(F)(F)F)cc1)cn2CCCNC(=O)C1(N)CCNCC1 ; NC1(C(=O)NCCCn2cc(-c3ccc(OC(F)(F)F)cc3)c3cc(CN4CC5CC4CN5Cc4ccccc4OC(F)(F)F)ccc32)CCNCC1 ; NC1(C(=O)NCCCn2cc(-c3ccc(OC(F)(F)F)cc3)c3cc(CN4CC5CC4CN5Cc4c(OC(F)(F)F)cccc4OC(F)(F)F)ccc32)CCNCC1 ; O=C(NCCCn1cc(-c2ccc(OC(F)(F)F)cc2)c2cc(CN3CC4CC3CN4Cc3ccccc3)ccc21)C1CNCCN1 ; Cc1ccccc1CN1CC2CC1CN2Cc1ccc2c(c1)c(-c1ccc(OC(F)(F)F)cc1)cn2CCCNC(=O)C1CNCCN1]

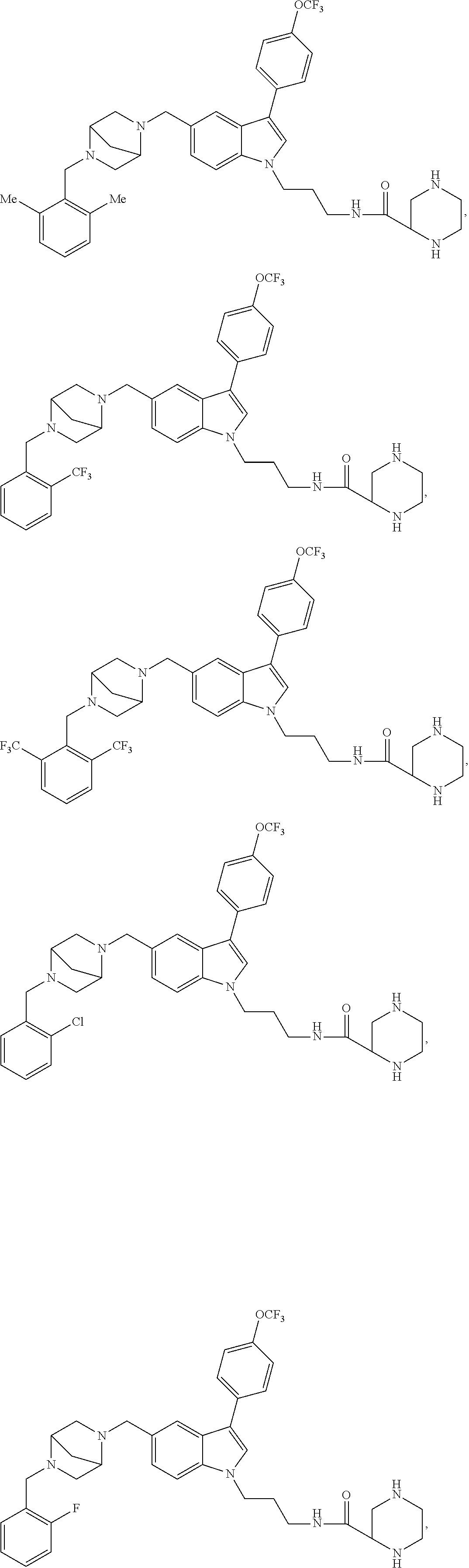 [SMILES: Cc1cccc(C)c1CN1CC2CC1CN2Cc1ccc2c(c1)c(-c1ccc(OC(F)(F)F)cc1)cn2CCCNC(=O)C1CNCCN1 ; O=C(NCCCn1cc(-c2ccc(OC(F)(F)F)cc2)c2cc(CN3CC4CC3CN4Cc3ccccc3C(F)(F)F)ccc21)C1CNCCN1 ; O=C(NCCCn1cc(-c2ccc(OC(F)(F)F)cc2)c2cc(CN3CC4CC3CN4Cc3c(C(F)(F)F)cccc3C(F)(F)F)ccc21)C1CNCCN1 ; O=C(NCCCn1cc(-c2ccc(OC(F)(F)F)cc2)c2cc(CN3CC4CC3CN4Cc3ccccc3Cl)ccc21)C1CNCCN1 ; O=C(NCCCn1cc(-c2ccc(OC(F)(F)F)cc2)c2cc(CN3CC4CC3CN4Cc3ccccc3F)ccc21)C1CNCCN1]

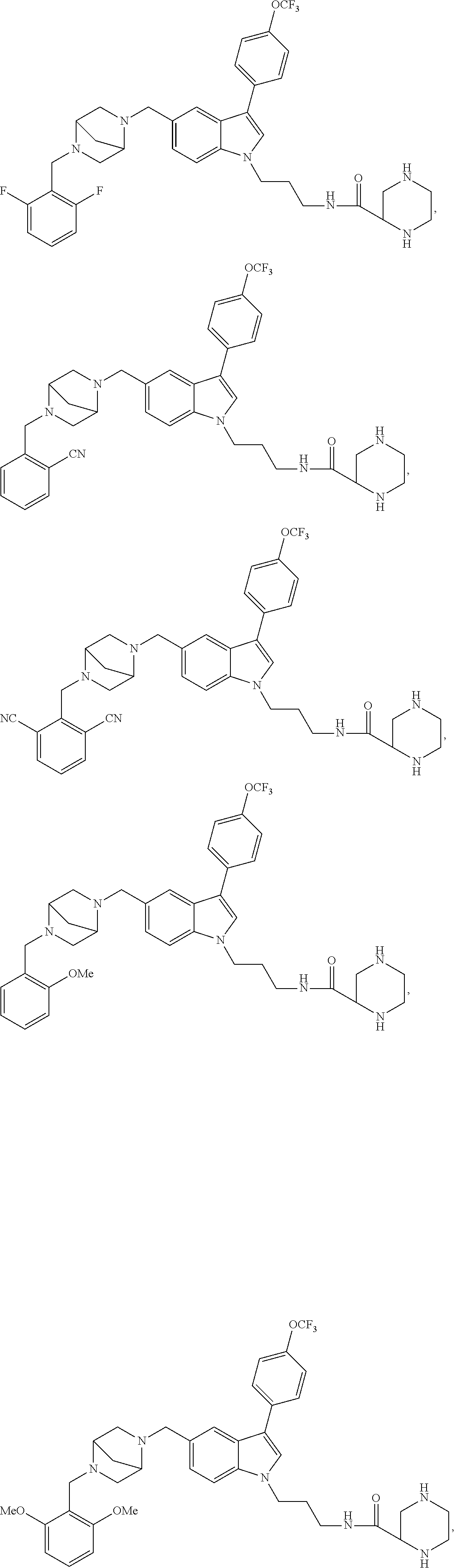 [SMILES: O=C(NCCCn1cc(-c2ccc(OC(F)(F)F)cc2)c2cc(CN3CC4CC3CN4Cc3c(F)cccc3F)ccc21)C1CNCCN1 ; N#Cc1ccccc1CN1CC2CC1CN2Cc1ccc2c(c1)c(-c1ccc(OC(F)(F)F)cc1)cn2CCCNC(=O)C1CNCCN1 ; N#Cc1cccc(C#N)c1CN1CC2CC1CN2Cc1ccc2c(c1)c(-c1ccc(OC(F)(F)F)cc1)cn2CCCNC(=O)C1CNCCN1 ; COc1ccccc1CN1CC2CC1CN2Cc1ccc2c(c1)c(-c1ccc(OC(F)(F)F)cc1)cn2CCCNC(=O)C1CNCCN1 ; COc1cccc(OC)c1CN1CC2CC1CN2Cc1ccc2c(c1)c(-c1ccc(OC(F)(F)F)cc1)cn2CCCNC(=O)C1CNCCN1]

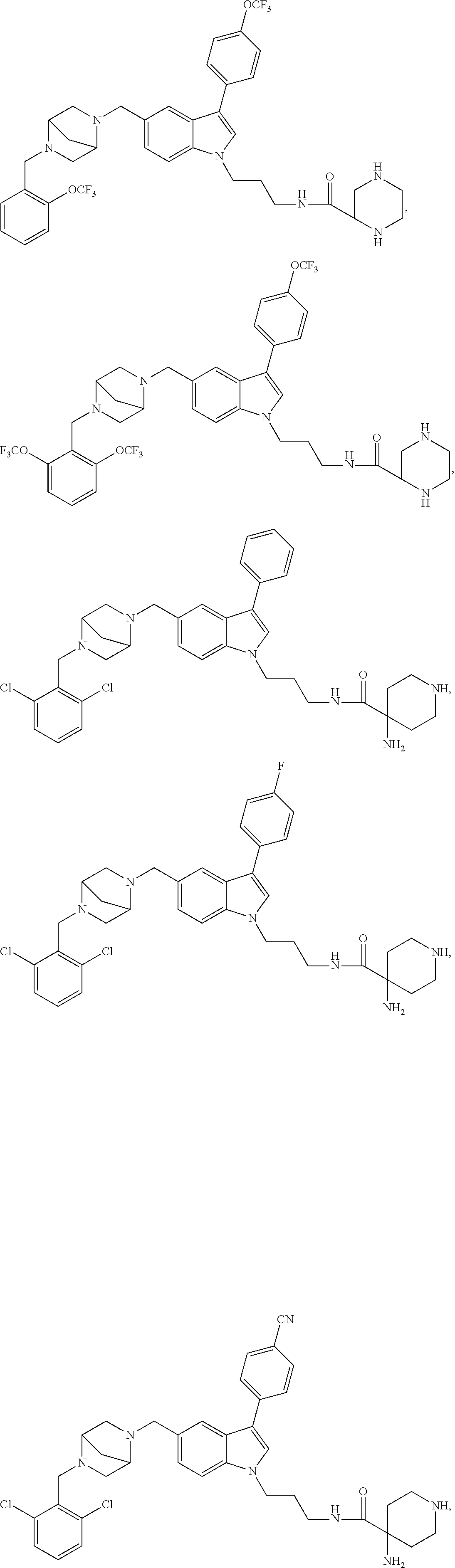 [SMILES: O=C(NCCCn1cc(-c2ccc(OC(F)(F)F)cc2)c2cc(CN3CC4CC3CN4Cc3ccccc3OC(F)(F)F)ccc21)C1CNCCN1 ; O=C(NCCCn1cc(-c2ccc(OC(F)(F)F)cc2)c2cc(CN3CC4CC3CN4Cc3c(OC(F)(F)F)cccc3OC(F)(F)F)ccc21)C1CNCCN1 ; NC1(C(=O)NCCCn2cc(-c3ccccc3)c3cc(CN4CC5CC4CN5Cc4c(Cl)cccc4Cl)ccc32)CCNCC1 ; NC1(C(=O)NCCCn2cc(-c3ccc(F)cc3)c3cc(CN4CC5CC4CN5Cc4c(Cl)cccc4Cl)ccc32)CCNCC1 ; N#Cc1ccc(-c2cn(CCCNC(=O)C3(N)CCNCC3)c3ccc(CN4CC5CC4CN5Cc4c(Cl)cccc4Cl)cc23)cc1]

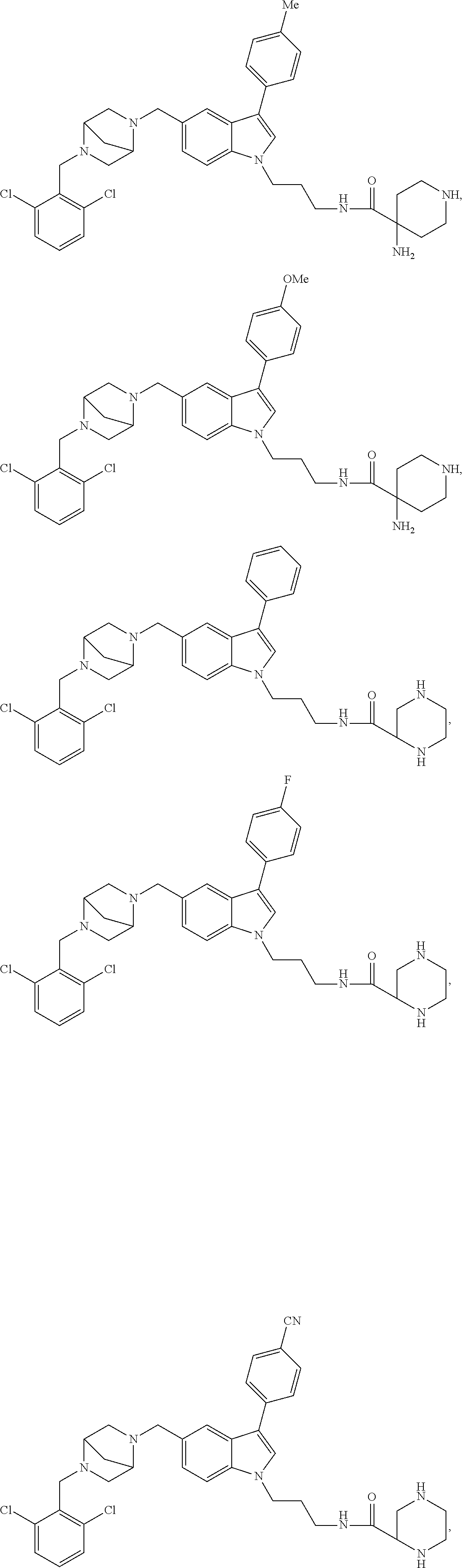 [SMILES: Cc1ccc(-c2cn(CCCNC(=O)C3(N)CCNCC3)c3ccc(CN4CC5CC4CN5Cc4c(Cl)cccc4Cl)cc23)cc1 ; COc1ccc(-c2cn(CCCNC(=O)C3(N)CCNCC3)c3ccc(CN4CC5CC4CN5Cc4c(Cl)cccc4Cl)cc23)cc1 ; O=C(NCCCn1cc(-c2ccccc2)c2cc(CN3CC4CC3CN4Cc3c(Cl)cccc3Cl)ccc21)C1CNCCN1 ; O=C(NCCCn1cc(-c2ccc(F)cc2)c2cc(CN3CC4CC3CN4Cc3c(Cl)cccc3Cl)ccc21)C1CNCCN1 ; N#Cc1ccc(-c2cn(CCCNC(=O)C3CNCCN3)c3ccc(CN4CC5CC4CN5Cc4c(Cl)cccc4Cl)cc23)cc1]

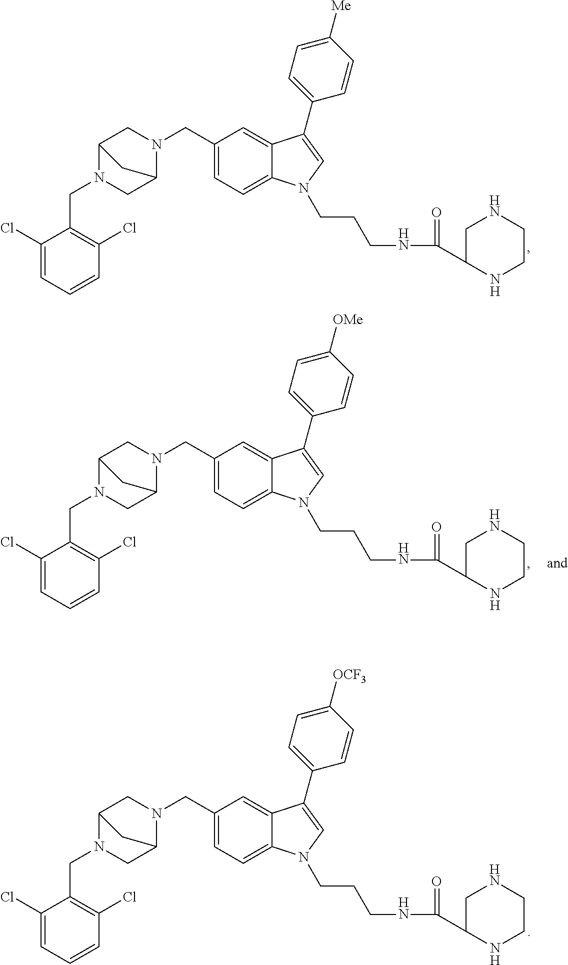 [SMILES: Cc1ccc(-c2cn(CCCNC(=O)C3CNCCN3)c3ccc(CN4CC5CC4CN5Cc4c(Cl)cccc4Cl)cc23)cc1 ; COc1ccc(-c2cn(CCCNC(=O)C3CNCCN3)c3ccc(CN4CC5CC4CN5Cc4c(Cl)cccc4Cl)cc23)cc1 ; O=C(NCCCn1cc(-c2ccc(OC(F)(F)F)cc2)c2cc(CN3CC4CC3CN4Cc3c(Cl)cccc3Cl)ccc21)C1CNCCN1]

In one aspect, provided herein is a compound of Formula (IIa), or a pharmaceutically acceptable salt, or solvate thereof:

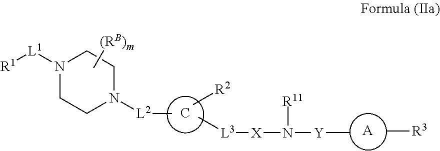

wherein,

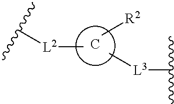

is a bicyclic heteroaryl that is selected from the following structures:

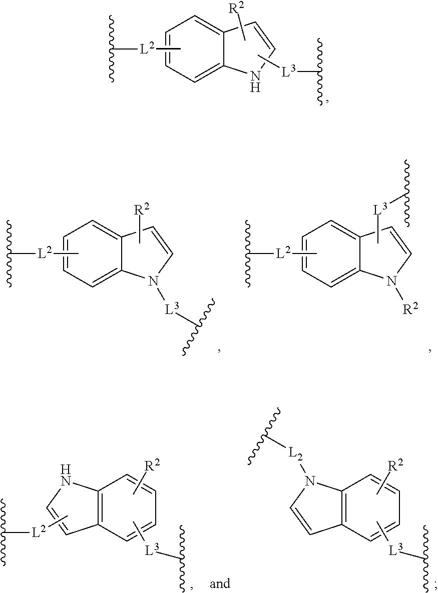

L¹ and L² are each independently an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is an optionally substituted aryl, optionally substituted heterocycloalkyl, or optionally substituted heteroaryl;
R² is an optionally substituted aryl, optionally substituted heterocycloalkyl, or optionally substituted heteroaryl;
each R^{B} is independently optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, a substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)— or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N; wherein if Ring A is substituted, then Ring A is substituted with at least one R^{A};
each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring, and
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring,
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); and
each m is independently 0, 1, 2, 3, or 4.

In some embodiments,

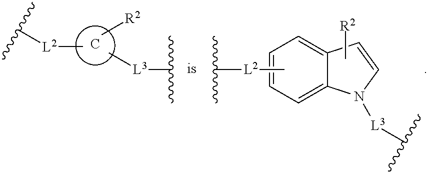

In some embodiments,

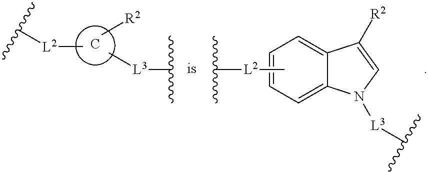

In some embodiments, a compound of Formula (IIa) has the following structure of Formula (IIb), or a pharmaceutically acceptable salt, or solvate thereof:

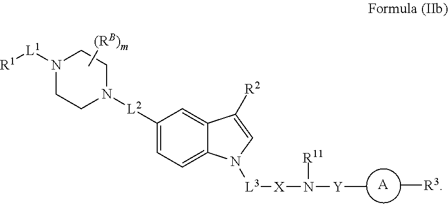

In some embodiments, a compound of Formula (IIa) has the following structure of Formula (IIc), or a pharmaceutically acceptable salt, or solvate thereof:

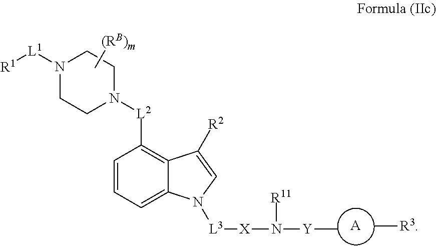

In some embodiments,

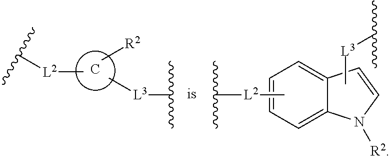

In some embodiments,

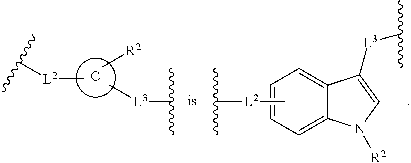

In some embodiments, a compound of Formula (IIa) has the following structure of Formula (IId), or a pharmaceutically acceptable salt, or solvate thereof:

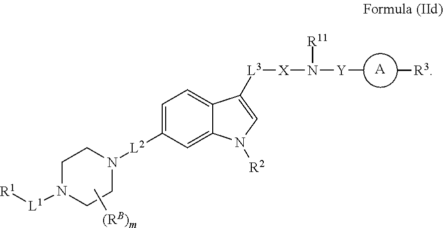

In some embodiments, a compound of Formula (IIa) has the following structure of Formula (IIe), or a pharmaceutically acceptable salt, or solvate thereof:

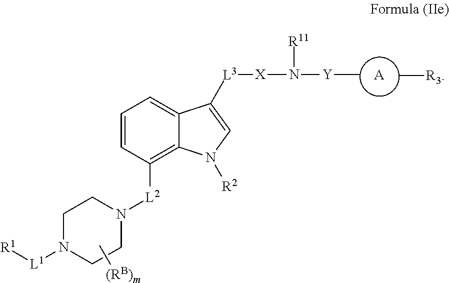

In some embodiments,

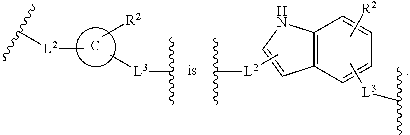

In some embodiments,

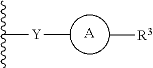

is selected from the following:

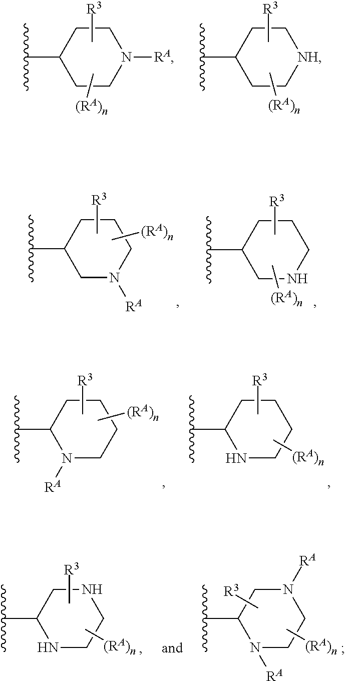

and each n is independently 0, 1, 2, 3, or 4.

In some embodiments,

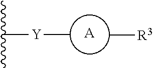

is selected from the following:

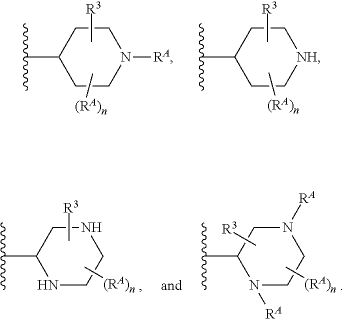

In some embodiments,

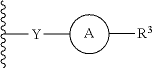

is selected from the following:

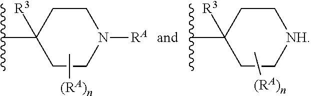

In some embodiments, L¹ is —CH₂—, C(═O), O, S, S(═O), S(═O)₂, or NR⁴. In some embodiments, L¹ is —CH_{2—}. In some embodiments, L² is —CH₂—, C(═O), O, S, S(═O), S(═O)₂, or NR⁴. In some embodiments, L² is —CH₂—.

In some embodiments, L³ is an optionally substituted C₁-C₆heteroalkylene, a substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene). In some embodiments, L³ is a substituted C₁-C₅alkylene. In some embodiments, L³ is:

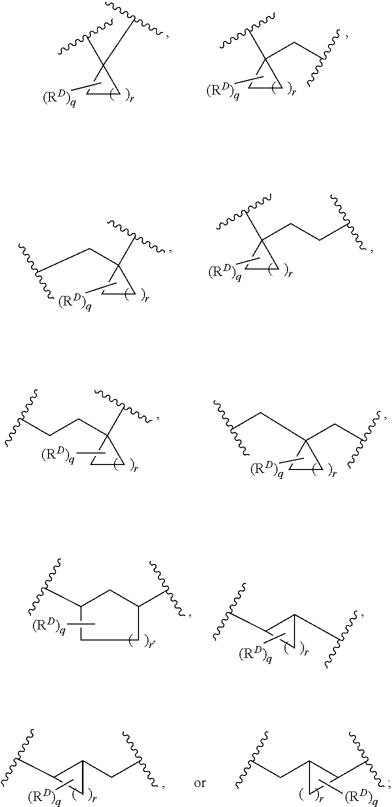

each q is independently 0, 1, 2, 3, or 4;

r is 1, 2, 3, 4, or 5, and

r′ is 1 or 2.

In some embodiments, X is —CH₂— or C(═O). In some embodiments, X is —CH₂—.

In some embodiments, X is:

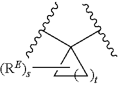

each s is independently 0, 1, 2, 3, or 4; and

t is 1, 2, 3, 4, or 5.

In some embodiments, X is:

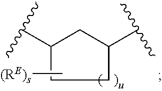

each s is independently 0, 1, 2, 3, or 4; and

u is 0, 1, or 2.

In some embodiments, Y is —CH₂— or C(═O). In some embodiments, Y is C(═O). In some embodiments, R¹¹ is hydrogen.

In some embodiments, R³ is H. In some embodiments, R³ is CH₂N(R⁹)(R¹⁰). In some embodiments, R³ is N(R⁹)(R¹⁰).

In some embodiments, R⁹ and R¹⁰ are each H. In some embodiments, R⁹ is H and R¹⁰ is —C₁-C₄alkylene-(optionally substituted phenyl) or —C₁-C₄alkylene-(optionally substituted heteroaryl). In some embodiments, R⁹ is H and R¹⁰ is —CH₂-(optionally substituted phenyl). In some embodiments, R⁹ is H and R¹⁰ is —CH₂-(optionally substituted heteroaryl).

In some embodiments, R³ is CH₂N(R⁹)(R¹⁰); and R⁹ and R¹⁰ are each H. In some embodiments, R³ is CH₂N(R⁹)(R¹⁰); and R⁹ is H and R¹⁰ is —C₁-C₄alkylene-(optionally substituted phenyl) or —C₁-C₄alkylene-(optionally substituted heteroaryl). In some embodiments, R³ is CH₂N(R⁹)(R¹⁰); and R⁹ is H and R¹⁰ is —C₁-C₄alkylene-(optionally substituted phenyl). In some embodiments, R³ is CH₂N(R⁹)(R¹⁰); and R⁹ is H and R¹⁰ is —C₁-C₄alkylene-(optionally substituted heteroaryl). In some embodiments, R³ is N(R⁹)(R¹⁰); and R⁹ and R¹⁰ are each H. In some embodiments, R³ is N(R⁹)(R¹⁰); and R⁹ is H and R¹⁰ is —C₁-C₄alkylene-(optionally substituted phenyl) or —C₁-C₄alkylene-(optionally substituted heteroaryl). In some embodiments, R³ is N(R⁹)(R¹⁰); and R⁹ is H and R¹⁰ is —C₁-C₄alkylene-(optionally substituted phenyl). In some embodiments, R³ is N(R⁹)(R¹⁰); and R⁹ is H and R¹⁰ is —C₁-C₄alkylene-(optionally substituted heteroaryl).

In some embodiments, R¹ is an unsubstituted phenyl. In some embodiments, R¹ is a substituted phenyl.

In some embodiments, R¹ is selected from:

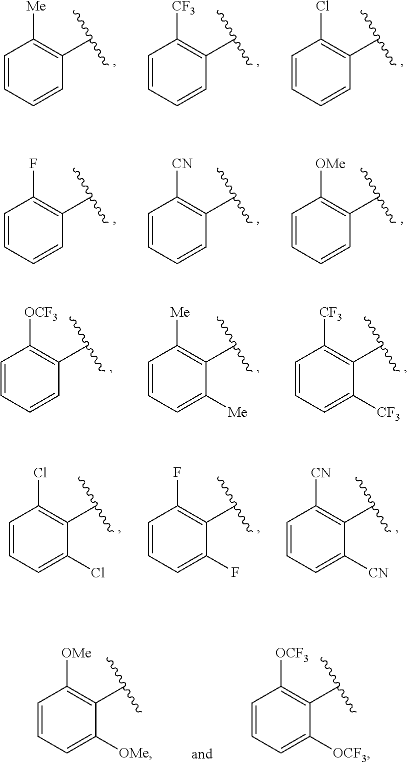

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

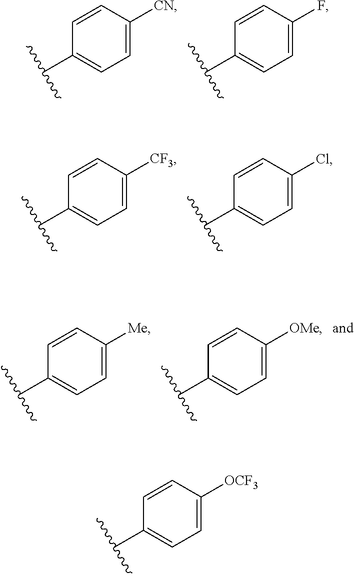

In some embodiments, R² is selected from:

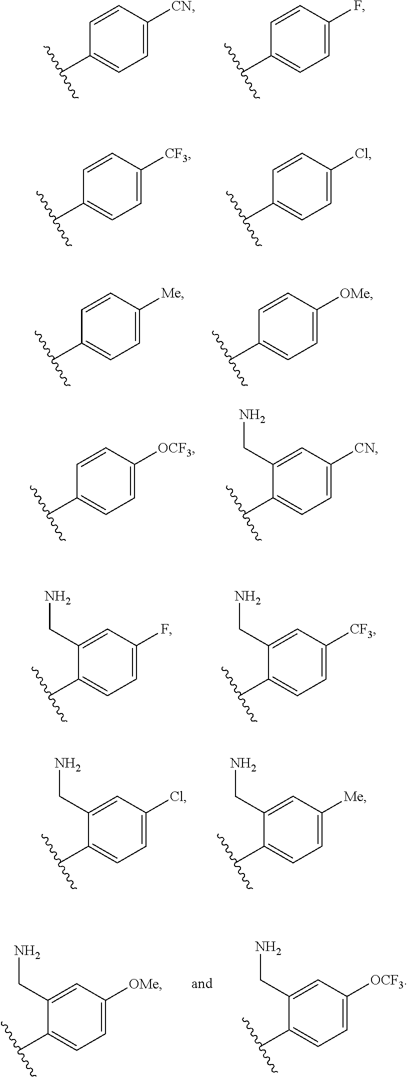

In some embodiments, R¹ is optionally substituted heterocycloalkyl. In some embodiments, R¹ is selected from:

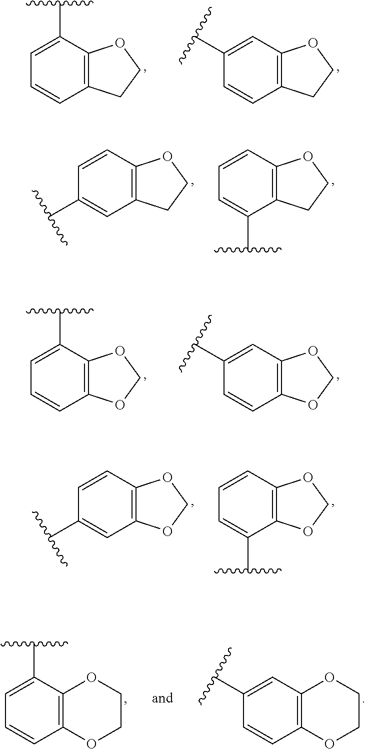

In some embodiments, R² is optionally substituted heterocycloalkyl. In some embodiments, R² is selected from:

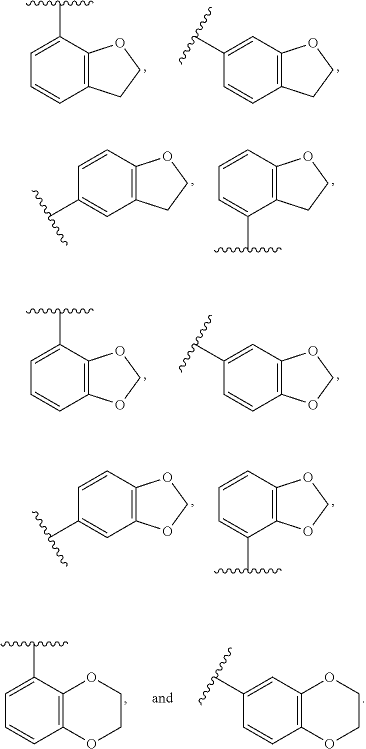

Also provided herein is a compound having a formula selected from:

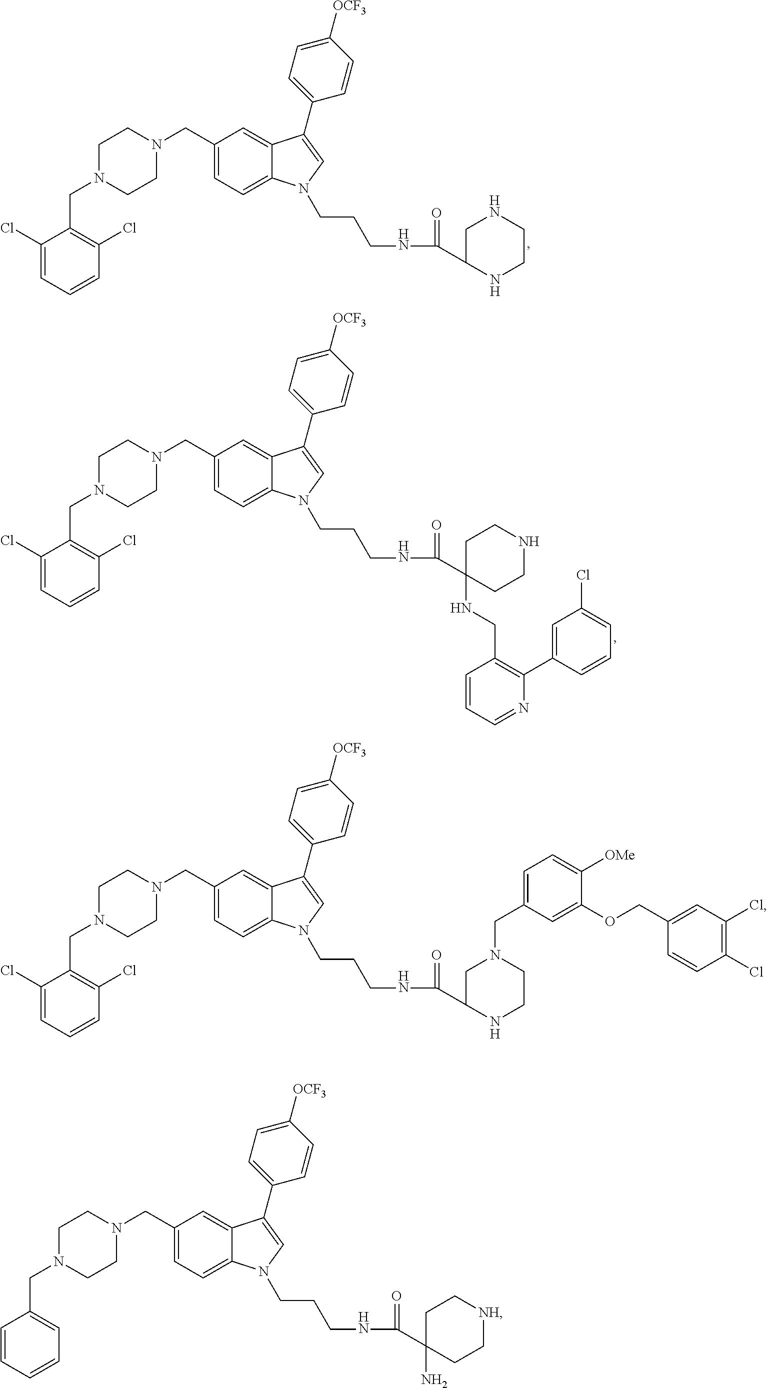 [SMILES: O=C(NCCCn1cc(-c2ccc(OC(F)(F)F)cc2)c2cc(CN3CCN(Cc4c(Cl)cccc4Cl)CC3)ccc21)C1CNCCN1 ; O=C(NCCCn1cc(-c2ccc(OC(F)(F)F)cc2)c2cc(CN3CCN(Cc4c(Cl)cccc4Cl)CC3)ccc21)C1(NCc2cccnc2-c2cccc(Cl)c2)CCNCC1 ; COc1ccc(CN2CCNC(C(=O)NCCCn3cc(-c4ccc(OC(F)(F)F)cc4)c4cc(CN5CCN(Cc6c(Cl)cccc6Cl)CC5)ccc43)C2)cc1OCc1ccc(Cl)c(Cl)c1 ; NC1(C(=O)NCCCn2cc(-c3ccc(OC(F)(F)F)cc3)c3cc(CN4CCN(Cc5ccccc5)CC4)ccc32)CCNCC1]

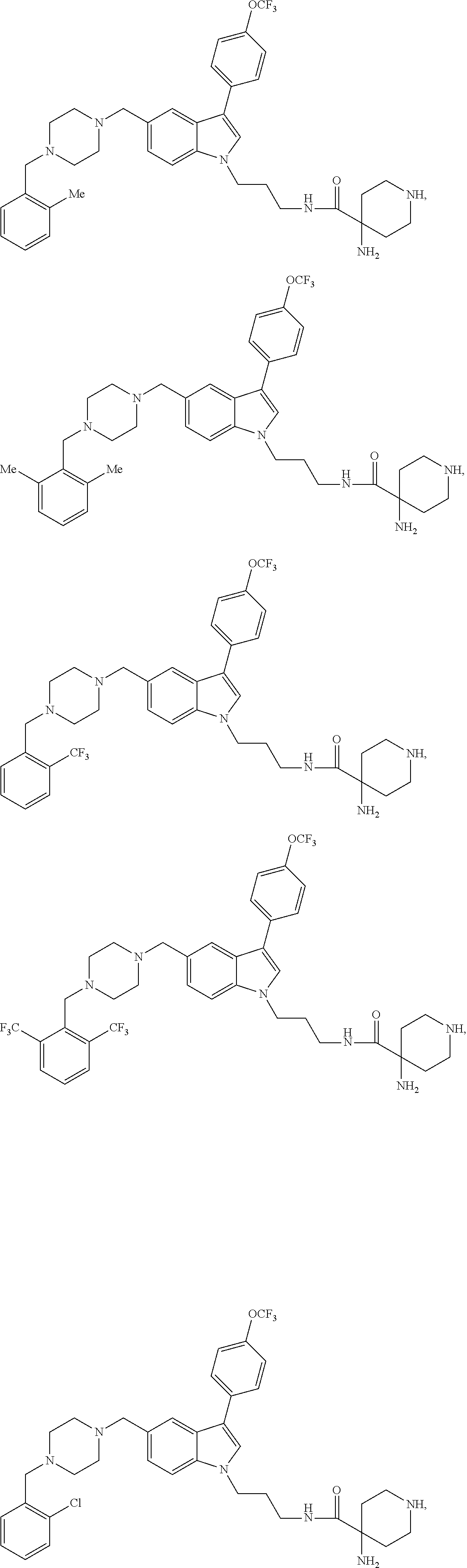 [SMILES: Cc1ccccc1CN1CCN(Cc2ccc3c(c2)c(-c2ccc(OC(F)(F)F)cc2)cn3CCCNC(=O)C2(N)CCNCC2)CC1 ; Cc1cccc(C)c1CN1CCN(Cc2ccc3c(c2)c(-c2ccc(OC(F)(F)F)cc2)cn3CCCNC(=O)C2(N)CCNCC2)CC1 ; NC1(C(=O)NCCCn2cc(-c3ccc(OC(F)(F)F)cc3)c3cc(CN4CCN(Cc5ccccc5C(F)(F)F)CC4)ccc32)CCNCC1 ; NC1(C(=O)NCCCn2cc(-c3ccc(OC(F)(F)F)cc3)c3cc(CN4CCN(Cc5c(C(F)(F)F)cccc5C(F)(F)F)CC4)ccc32)CCNCC1 ; NC1(C(=O)NCCCn2cc(-c3ccc(OC(F)(F)F)cc3)c3cc(CN4CCN(Cc5ccccc5Cl)CC4)ccc32)CCNCC1]

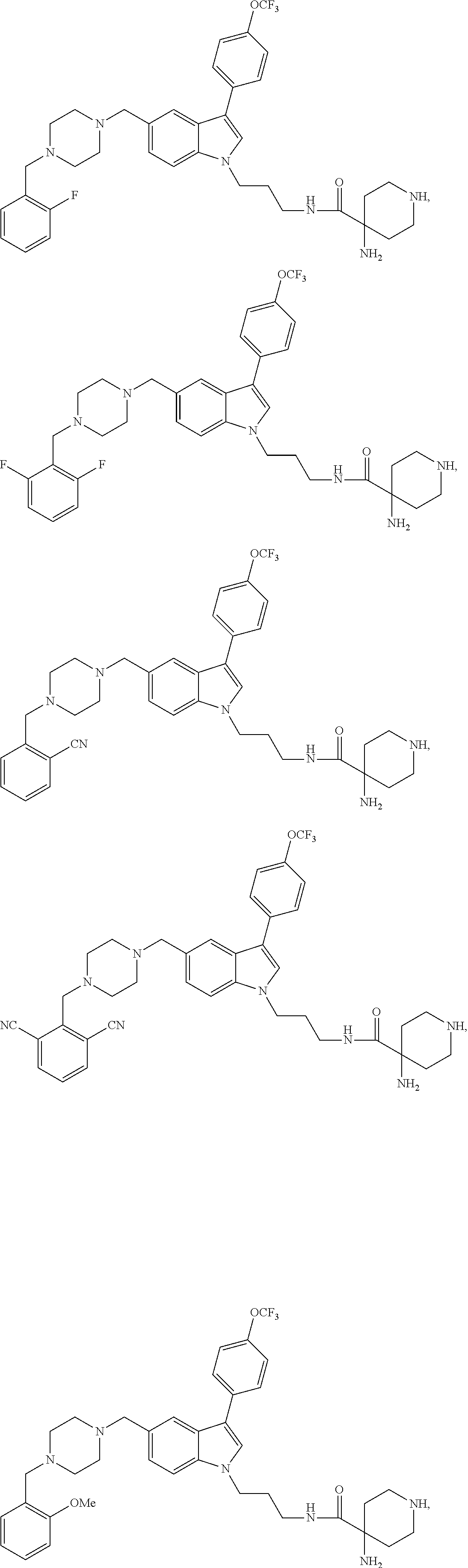 [SMILES: NC1(C(=O)NCCCn2cc(-c3ccc(OC(F)(F)F)cc3)c3cc(CN4CCN(Cc5ccccc5F)CC4)ccc32)CCNCC1 ; NC1(C(=O)NCCCn2cc(-c3ccc(OC(F)(F)F)cc3)c3cc(CN4CCN(Cc5c(F)cccc5F)CC4)ccc32)CCNCC1 ; N#Cc1ccccc1CN1CCN(Cc2ccc3c(c2)c(-c2ccc(OC(F)(F)F)cc2)cn3CCCNC(=O)C2(N)CCNCC2)CC1 ; N#Cc1cccc(C#N)c1CN1CCN(Cc2ccc3c(c2)c(-c2ccc(OC(F)(F)F)cc2)cn3CCCNC(=O)C2(N)CCNCC2)CC1 ; COc1ccccc1CN1CCN(Cc2ccc3c(c2)c(-c2ccc(OC(F)(F)F)cc2)cn3CCCNC(=O)C2(N)CCNCC2)CC1]

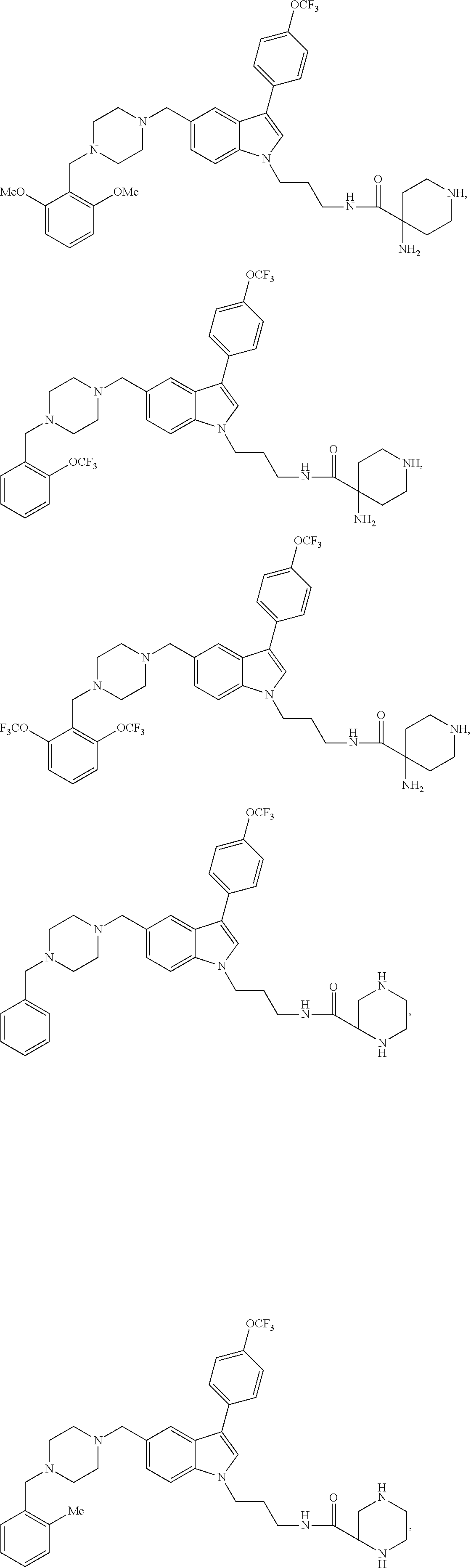 [SMILES: COc1cccc(OC)c1CN1CCN(Cc2ccc3c(c2)c(-c2ccc(OC(F)(F)F)cc2)cn3CCCNC(=O)C2(N)CCNCC2)CC1 ; NC1(C(=O)NCCCn2cc(-c3ccc(OC(F)(F)F)cc3)c3cc(CN4CCN(Cc5ccccc5OC(F)(F)F)CC4)ccc32)CCNCC1 ; NC1(C(=O)NCCCn2cc(-c3ccc(OC(F)(F)F)cc3)c3cc(CN4CCN(Cc5c(OC(F)(F)F)cccc5OC(F)(F)F)CC4)ccc32)CCNCC1 ; O=C(NCCCn1cc(-c2ccc(OC(F)(F)F)cc2)c2cc(CN3CCN(Cc4ccccc4)CC3)ccc21)C1CNCCN1 ; Cc1ccccc1CN1CCN(Cc2ccc3c(c2)c(-c2ccc(OC(F)(F)F)cc2)cn3CCCNC(=O)C2CNCCN2)CC1]

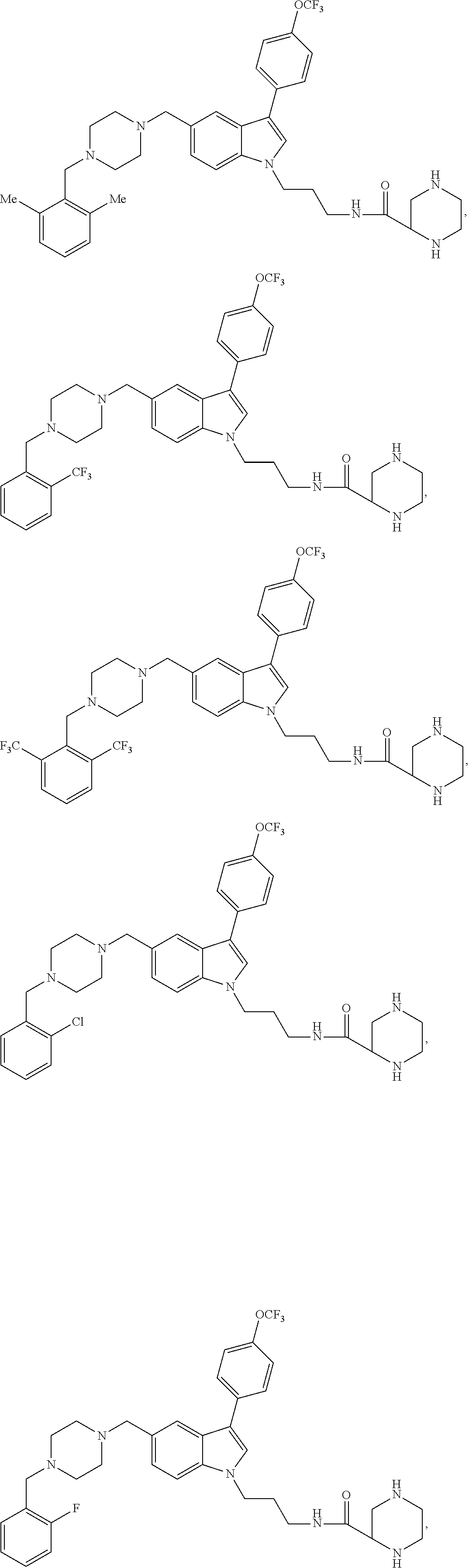 [SMILES: Cc1cccc(C)c1CN1CCN(Cc2ccc3c(c2)c(-c2ccc(OC(F)(F)F)cc2)cn3CCCNC(=O)C2CNCCN2)CC1 ; O=C(NCCCn1cc(-c2ccc(OC(F)(F)F)cc2)c2cc(CN3CCN(Cc4ccccc4C(F)(F)F)CC3)ccc21)C1CNCCN1 ; O=C(NCCCn1cc(-c2ccc(OC(F)(F)F)cc2)c2cc(CN3CCN(Cc4c(C(F)(F)F)cccc4C(F)(F)F)CC3)ccc21)C1CNCCN1 ; O=C(NCCCn1cc(-c2ccc(OC(F)(F)F)cc2)c2cc(CN3CCN(Cc4ccccc4Cl)CC3)ccc21)C1CNCCN1 ; O=C(NCCCn1cc(-c2ccc(OC(F)(F)F)cc2)c2cc(CN3CCN(Cc4ccccc4F)CC3)ccc21)C1CNCCN1]

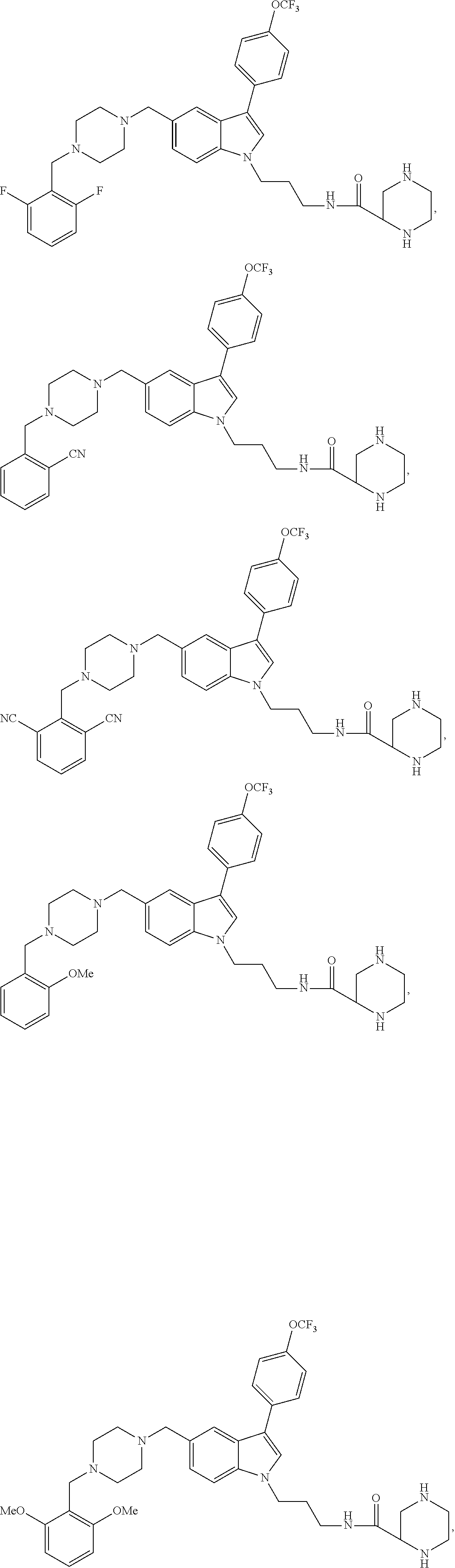 [SMILES: O=C(NCCCn1cc(-c2ccc(OC(F)(F)F)cc2)c2cc(CN3CCN(Cc4c(F)cccc4F)CC3)ccc21)C1CNCCN1 ; N#Cc1ccccc1CN1CCN(Cc2ccc3c(c2)c(-c2ccc(OC(F)(F)F)cc2)cn3CCCNC(=O)C2CNCCN2)CC1 ; N#Cc1cccc(C#N)c1CN1CCN(Cc2ccc3c(c2)c(-c2ccc(OC(F)(F)F)cc2)cn3CCCNC(=O)C2CNCCN2)CC1 ; COc1ccccc1CN1CCN(Cc2ccc3c(c2)c(-c2ccc(OC(F)(F)F)cc2)cn3CCCNC(=O)C2CNCCN2)CC1 ; COc1cccc(OC)c1CN1CCN(Cc2ccc3c(c2)c(-c2ccc(OC(F)(F)F)cc2)cn3CCCNC(=O)C2CNCCN2)CC1]

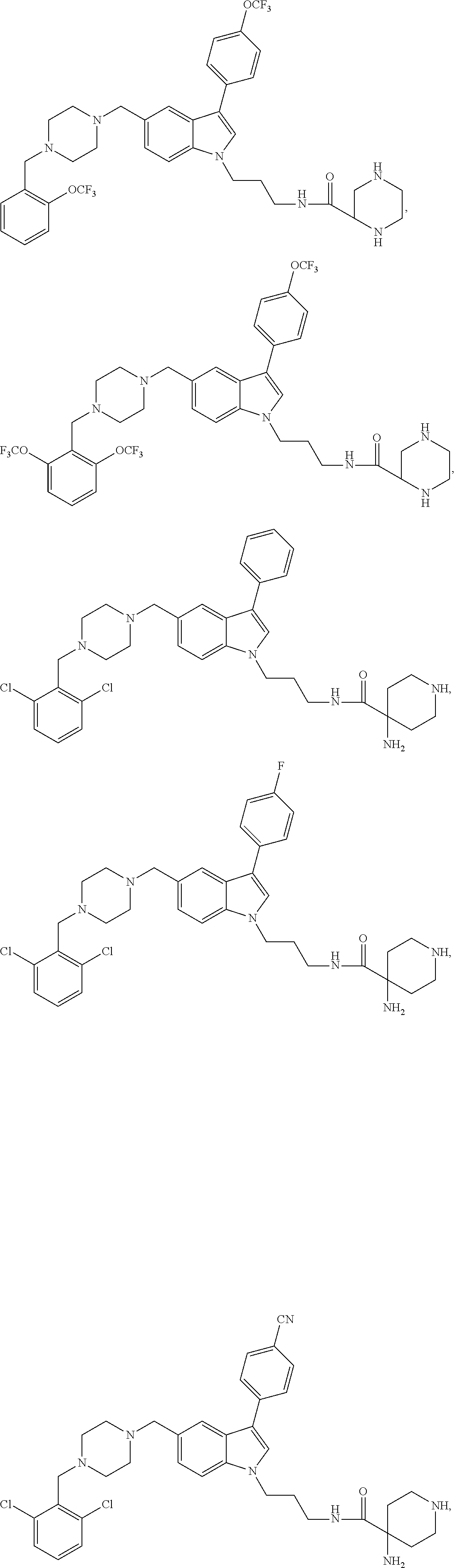 [SMILES: O=C(NCCCn1cc(-c2ccc(OC(F)(F)F)cc2)c2cc(CN3CCN(Cc4ccccc4OC(F)(F)F)CC3)ccc21)C1CNCCN1 ; O=C(NCCCn1cc(-c2ccc(OC(F)(F)F)cc2)c2cc(CN3CCN(Cc4c(OC(F)(F)F)cccc4OC(F)(F)F)CC3)ccc21)C1CNCCN1 ; NC1(C(=O)NCCCn2cc(-c3ccccc3)c3cc(CN4CCN(Cc5c(Cl)cccc5Cl)CC4)ccc32)CCNCC1 ; NC1(C(=O)NCCCn2cc(-c3ccc(F)cc3)c3cc(CN4CCN(Cc5c(Cl)cccc5Cl)CC4)ccc32)CCNCC1 ; N#Cc1ccc(-c2cn(CCCNC(=O)C3(N)CCNCC3)c3ccc(CN4CCN(Cc5c(Cl)cccc5Cl)CC4)cc23)cc1]

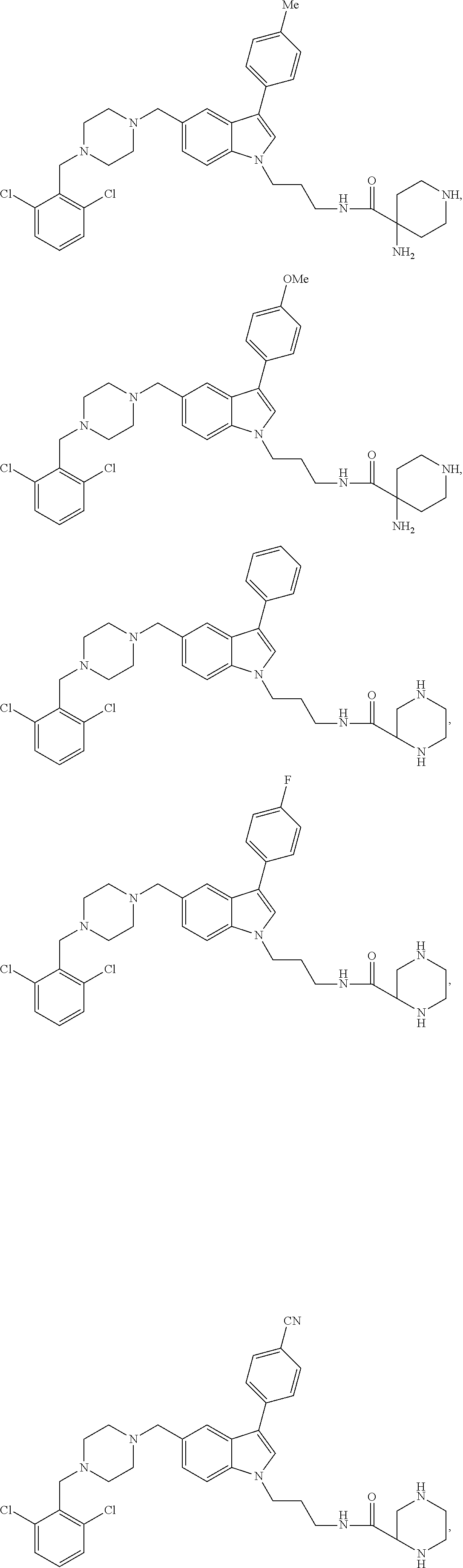 [SMILES: Cc1ccc(-c2cn(CCCNC(=O)C3(N)CCNCC3)c3ccc(CN4CCN(Cc5c(Cl)cccc5Cl)CC4)cc23)cc1 ; COc1ccc(-c2cn(CCCNC(=O)C3(N)CCNCC3)c3ccc(CN4CCN(Cc5c(Cl)cccc5Cl)CC4)cc23)cc1 ; O=C(NCCCn1cc(-c2ccccc2)c2cc(CN3CCN(Cc4c(Cl)cccc4Cl)CC3)ccc21)C1CNCCN1 ; O=C(NCCCn1cc(-c2ccc(F)cc2)c2cc(CN3CCN(Cc4c(Cl)cccc4Cl)CC3)ccc21)C1CNCCN1 ; N#Cc1ccc(-c2cn(CCCNC(=O)C3CNCCN3)c3ccc(CN4CCN(Cc5c(Cl)cccc5Cl)CC4)cc23)cc1]

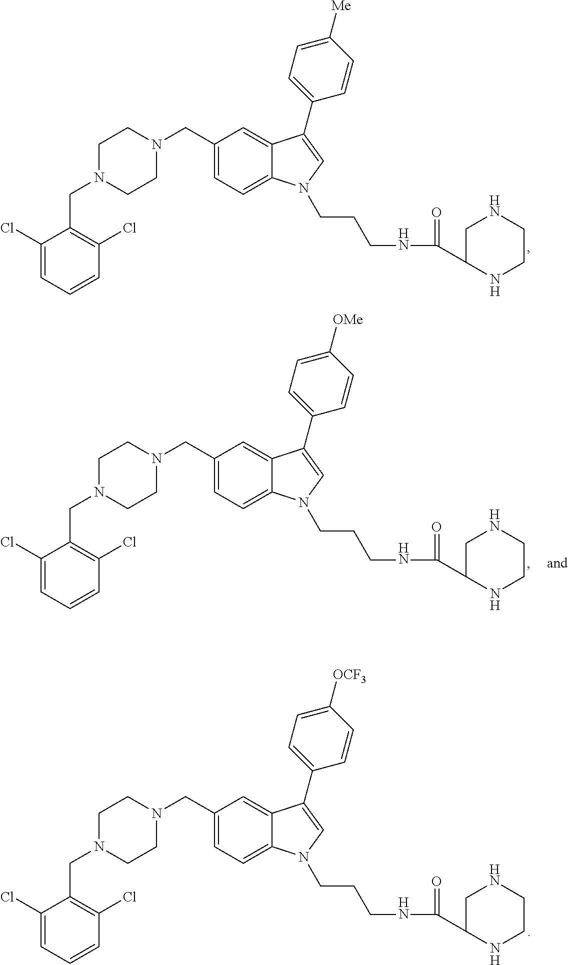 [SMILES: Cc1ccc(-c2cn(CCCNC(=O)C3CNCCN3)c3ccc(CN4CCN(Cc5c(Cl)cccc5Cl)CC4)cc23)cc1 ; COc1ccc(-c2cn(CCCNC(=O)C3CNCCN3)c3ccc(CN4CCN(Cc5c(Cl)cccc5Cl)CC4)cc23)cc1 ; O=C(NCCCn1cc(-c2ccc(OC(F)(F)F)cc2)c2cc(CN3CCN(Cc4c(Cl)cccc4Cl)CC3)ccc21)C1CNCCN1]

In another aspect provided herein is a compound of Formula (IIIa), or a pharmaceutically acceptable salt, or solvate thereof:

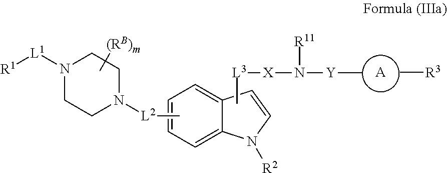

wherein,
L¹ and L² are each independently an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is an optionally substituted aryl, optionally substituted heterocycloalkyl, or optionally substituted heteroaryl;
R² is an optionally substituted aryl, optionally substituted heterocycloalkyl, or optionally substituted heteroaryl;
each R^{B} is independently optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N; wherein if Ring A is substituted, then Ring A is substituted with at least one R^{A};
each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring,
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); and
each m is independently 0, 1, 2, 3, or 4.

In some embodiments, the compound has the following structure of Formula (IIIb), or a pharmaceutically acceptable salt, or solvate thereof:

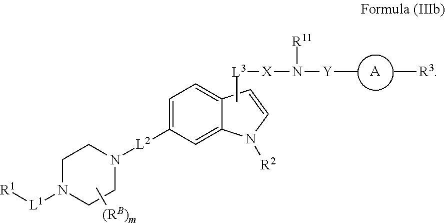

In some embodiments, the compound has the following structure of Formula (IIIc), or a pharmaceutically acceptable salt, or solvate thereof:

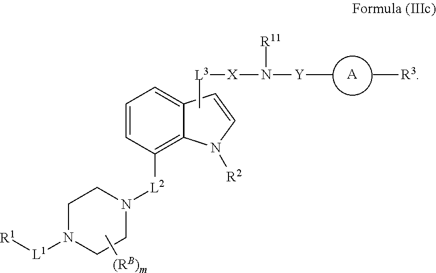

In some embodiments,

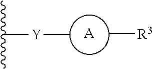

is selected from the following:

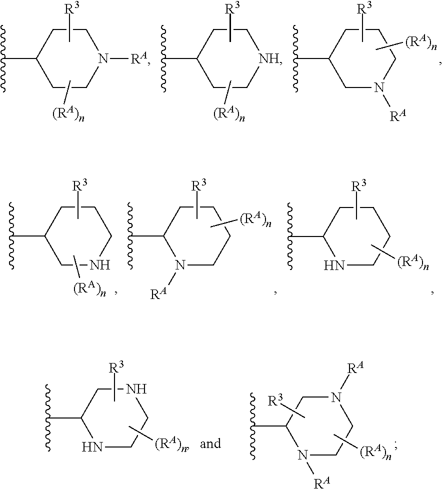

and each n is independently 0, 1, 2, 3, or 4.

In some embodiments,

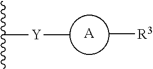

is selected from the following:

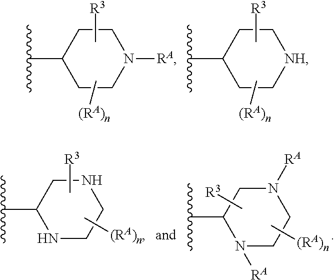

In some embodiments,

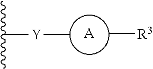

is selected from the following:

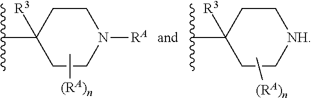

In some embodiments, L¹ is —CH₂—, C(═O), O, S, S(═O), S(═O)₂, or NR⁴. In some embodiments, L¹ is —CH_{2—}. In some embodiments, L² is —CH₂—, C(═O), O, S, S(═O), S(═O)₂, or NR⁴. In some embodiments, L² is —CH₂—. In some embodiments, L³ is absent, —CH₂—, —CH₂—CH₂—, or —CH₂—CH₂—CH₂—. In some embodiments, L³ is —CH₂—CH₂—.

In some embodiments, L³ is:

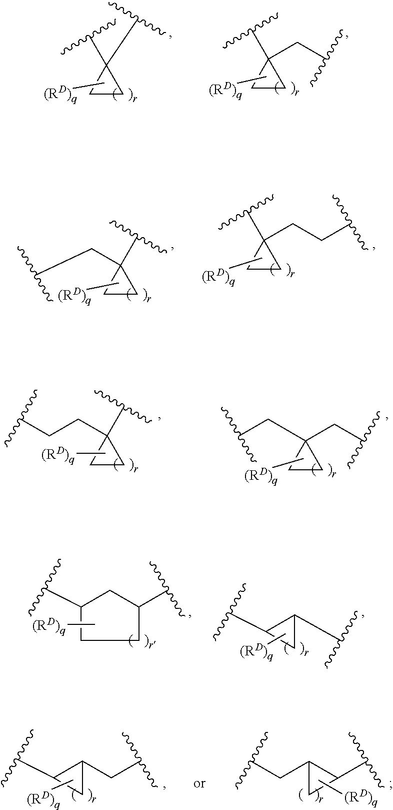

each q is independently 0, 1, 2, 3, or 4;

r is 1, 2, 3, or 4; and

r′ is 1 or 2.

In some embodiments, X is —CH₂— or C(═O). In some embodiments, X is —CH₂—.

In some embodiments, X is:

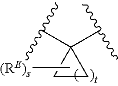

each s is independently 0, 1, 2, 3, or 4; and

t is 1, 2, 3, or 4.

In some embodiments, X is:

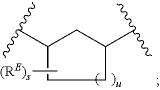

each s is independently 0, 1, 2, 3, or 4; and

u is 0, 1, or 2.

In some embodiments, L³-X is —CH₂—CH₂—CH₂—. In some embodiments, Y is —CH₂— or C(═O). In some embodiments, Y is C(═O). In some embodiments, R¹¹ is hydrogen. In some embodiments, R⁹ and R¹⁰ are each H. In some embodiments, R⁹ is H and R¹⁰ is —C₁-C₄alkylene-(optionally substituted phenyl) or —C₁-C₄alkylene-(optionally substituted heteroaryl). In some embodiments, R⁹ is H and R¹⁰ is —CH₂-(optionally substituted phenyl). In some embodiments, R⁹ is H and R¹⁰ is —CH₂-(optionally substituted heteroaryl). In some embodiments, R¹ is an unsubstituted phenyl. In some embodiments, R¹ is a substituted phenyl. In some embodiments, R¹ is selected from:

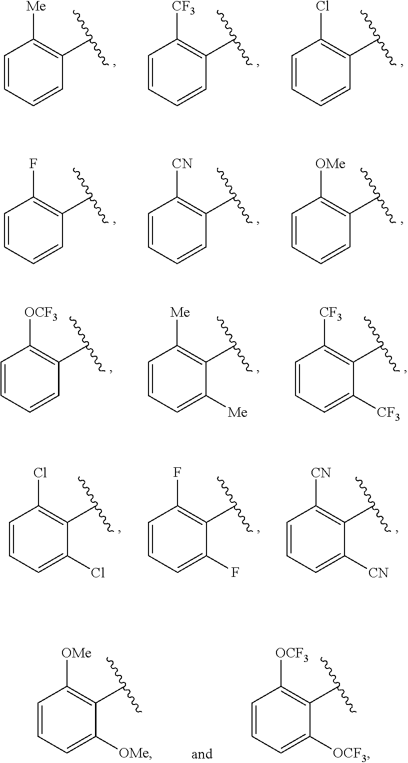

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

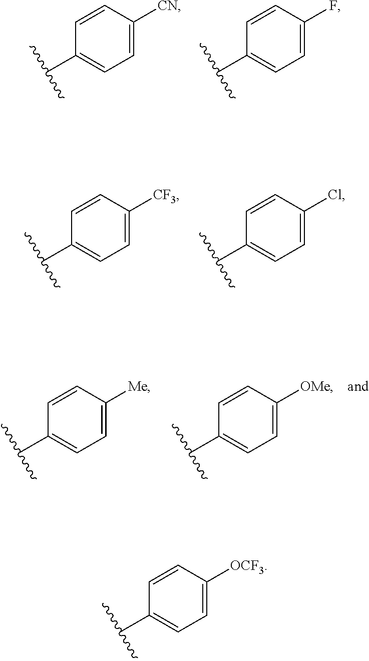

In some embodiments, R² is selected from:

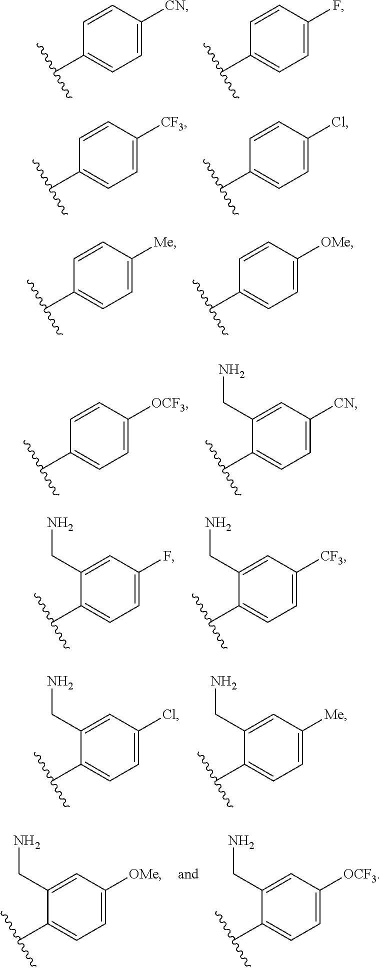

In some embodiments, R¹ is optionally substituted heterocycloalkyl. In some embodiments, R¹ is selected from:

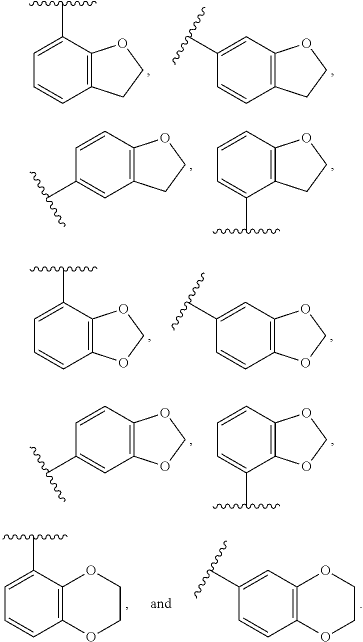

In some embodiments, R² is optionally substituted heterocycloalkyl. In some embodiments, R² is selected from:

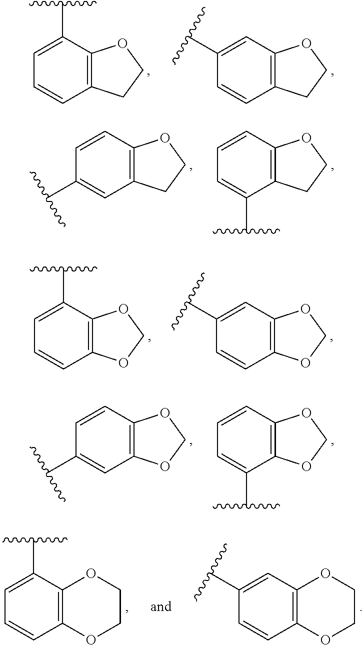

In some embodiments, the compound of Formula (IIIa) is selected from:

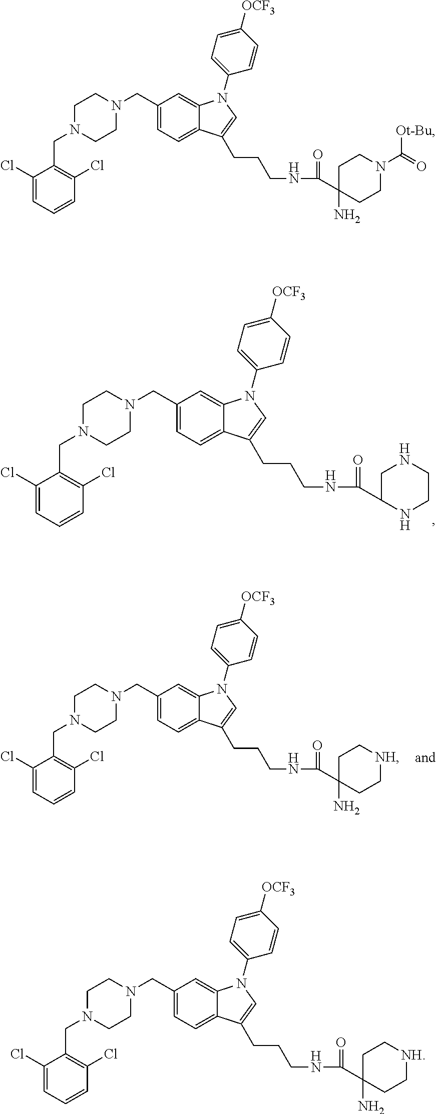 [SMILES: CC(C)(C)OC(=O)N1CCC(N)(C(=O)NCCCc2cn(-c3ccc(OC(F)(F)F)cc3)c3cc(CN4CCN(Cc5c(Cl)cccc5Cl)CC4)ccc23)CC1 ; O=C(NCCCc1cn(-c2ccc(OC(F)(F)F)cc2)c2cc(CN3CCN(Cc4c(Cl)cccc4Cl)CC3)ccc12)C1CNCCN1 ; NC1(C(=O)NCCCc2cn(-c3ccc(OC(F)(F)F)cc3)c3cc(CN4CCN(Cc5c(Cl)cccc5Cl)CC4)ccc23)CCNCC1 ; "and" ; NC1(C(=O)NCCCc2cn(-c3ccc(OC(F)(F)F)cc3)c3cc(CN4CCN(Cc5c(Cl)cccc5Cl)CC4)ccc23)CCNCC1]

In another aspect provided herein is a compound of Formula (IVa), or a pharmaceutically acceptable salt, or solvate thereof:

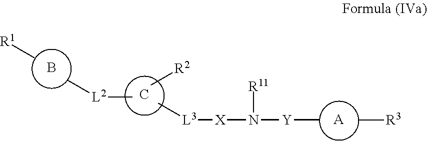

wherein,

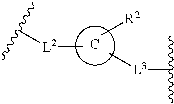

is a bicyclic heteroaryl that is selected from the following structures:

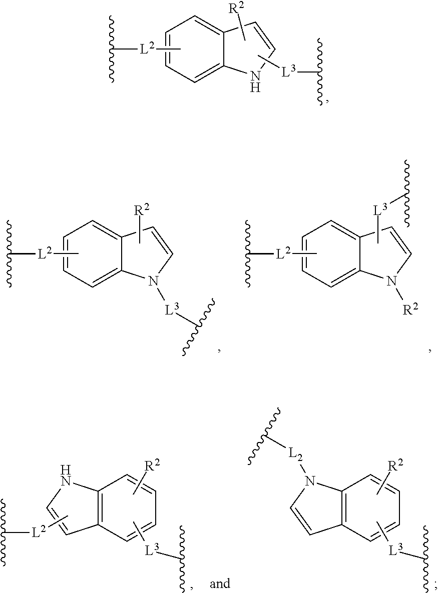

L² is an optionally substituted C₁-C₆alkylene, an optionally substituted C₁-C₆heteroalkylene, an optionally substituted C₃-C₆cycloalkylene, C(═O), O, S, S(═O),
S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N;
wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N;
wherein if Ring A is substituted, then Ring A is substituted with at least one R^{A};
each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring,
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl).

In some embodiments,

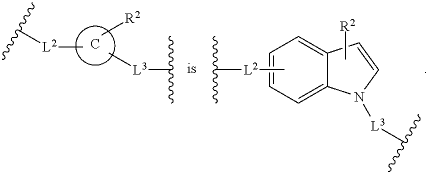

In some embodiments,

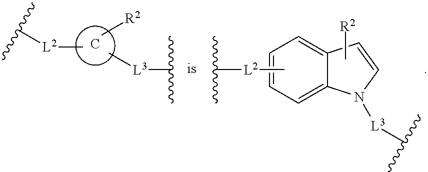

In some embodiments, the compound has the following structure of Formula (IVb), or a pharmaceutically acceptable salt, or solvate thereof:

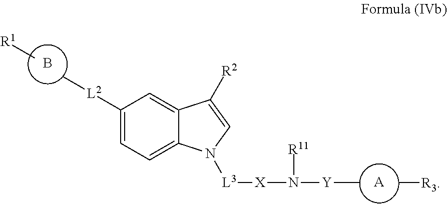

In some embodiments, the compound has the following structure of Formula (IVc), or a pharmaceutically acceptable salt, or solvate thereof:

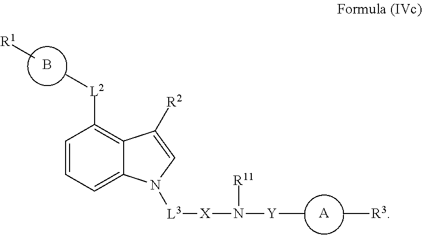

In some embodiments,

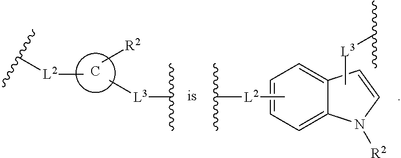

In some embodiments,

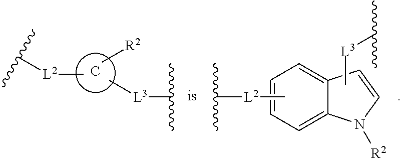

In some embodiments, the compound has the following structure of Formula (IVd), or a pharmaceutically acceptable salt, or solvate thereof:

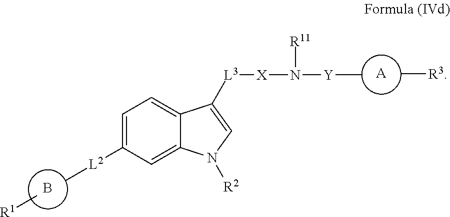

In some embodiments, the compound has the following structure of Formula (IVe), or a pharmaceutically acceptable salt, or solvate thereof:

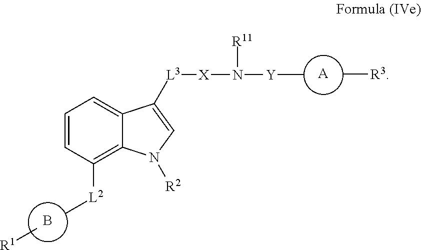

In some embodiments,

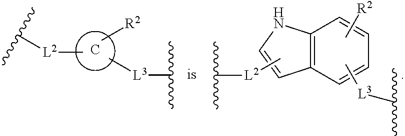

In some embodiments,

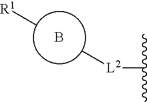

is selected from the following:

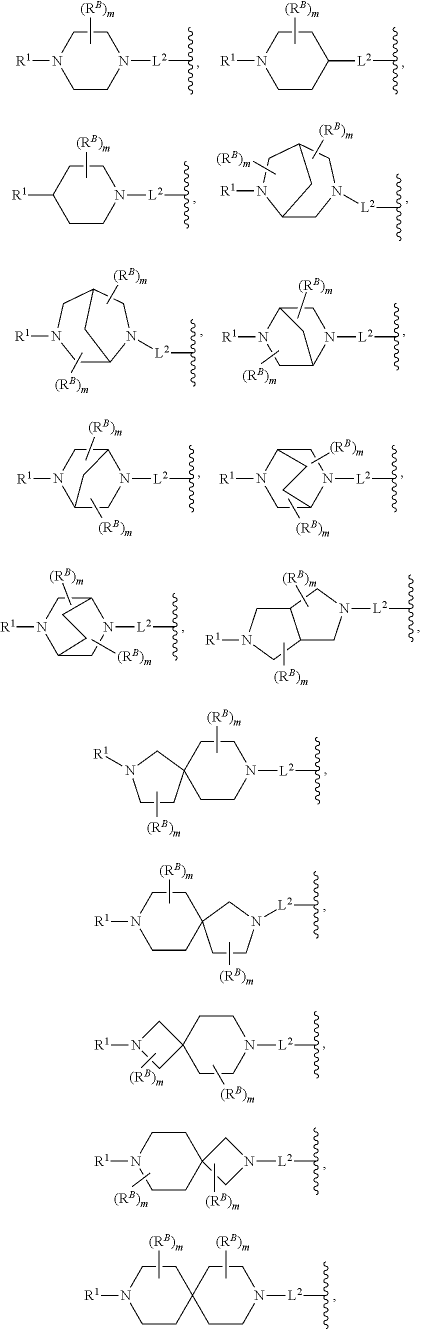

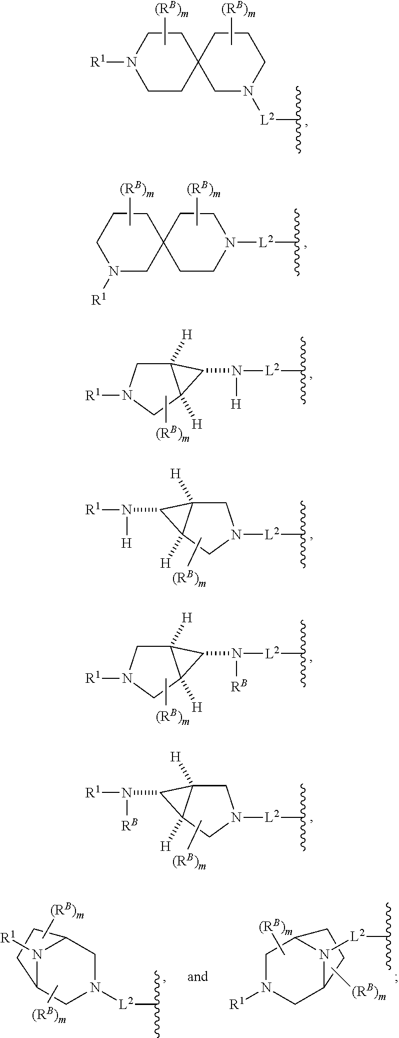

and each m is independently 0, 1, 2, 3, or 4.

In some embodiments,

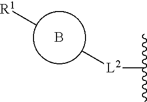

is selected from the following:

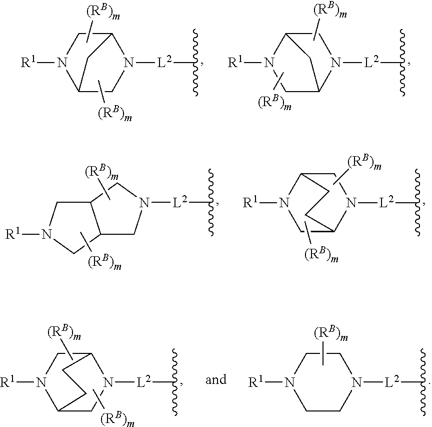

In some embodiments,

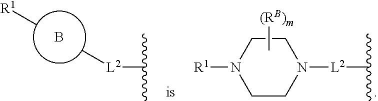

In some embodiments,

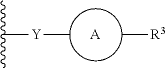

is selected from the following:

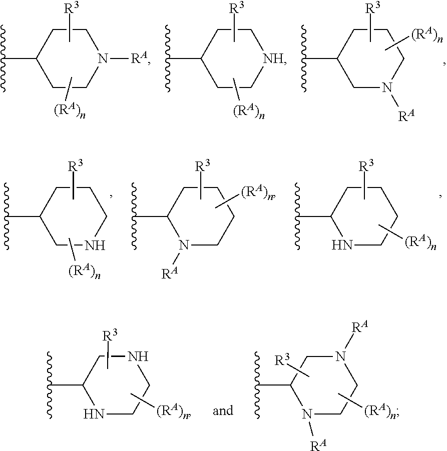

and each n is independently 0, 1, 2, 3, or 4.

In some embodiments,

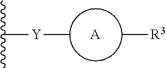

is selected from the following:

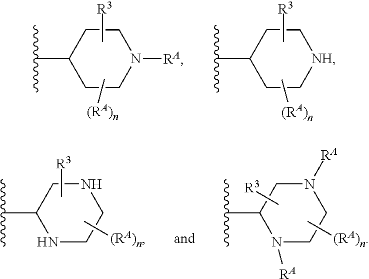

In some embodiments,

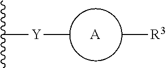

is selected from the following:

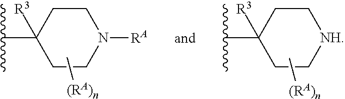

In some embodiments, L² is —CH₂—, C(═O), O, S, S(═O), S(═O)₂, or NR⁴. In some embodiments, L² is —CH₂—. In some embodiments, L³ is absent, —CH₂—, —CH₂—CH₂—, or —CH₂—CH₂—CH₂—. In some embodiments, L³ is —CH₂—CH₂—.

In some embodiments, L³ is:

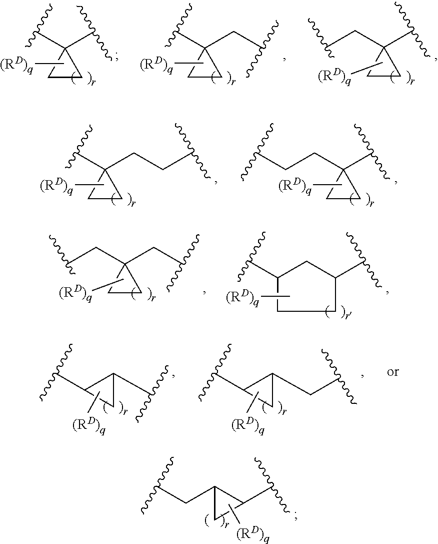

each q is independently 0, 1, 2, 3, or 4;

r is 1, 2, 3, or 4; and

r′ is 1 or 2.

In some embodiments, X is —CH₂— or C(═O). In some embodiments, X is —CH₂—. In some embodiments, X is:

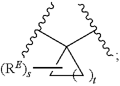

each s is independently 0, 1, 2, 3, or 4; and

t is 1, 2, 3, or 4.

In some embodiments, X is:

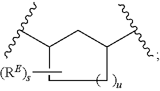

and

each s is independently 0, 1, 2, 3, or 4; and

u is 0, 1, or 2.

In some embodiments, L³-X is —CH₂—CH₂—CH₂—. In some embodiments, Y is —CH₂— or C(═O). In some embodiments, Y is C(═O). In some embodiments, R¹¹ is hydrogen. In some embodiments, R⁹ and R¹⁰ are each H. In some embodiments, R⁹ is H and R¹⁰ is —C₁-C₄alkylene-(optionally substituted phenyl) or —C₁-C₄alkylene-(optionally substituted heteroaryl). In some embodiments, R⁹ is H and R¹⁰ is —CH₂-(optionally substituted phenyl). In some embodiments, R⁹ is H and R¹⁰ is —CH₂-(optionally substituted heteroaryl). In some embodiments, R¹ is an hydrogen, an optionally substituted C₁-C₆alkyl, or an optionally substituted aryl. In some embodiments, R¹ is hydrogen. In some embodiments, R¹ is an unsubstituted C₁-C₆alkyl. In some embodiments, R¹ is a substituted C₁-C₆alkyl. In some embodiments, R¹ is an unsubstituted phenyl. In some embodiments, R¹ is a substituted phenyl. In some embodiments, R¹ is selected from:

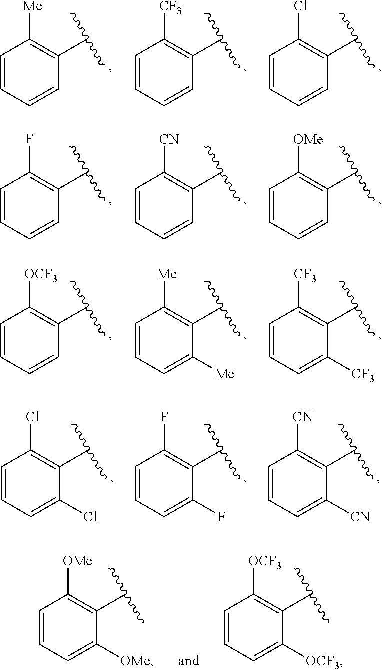

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

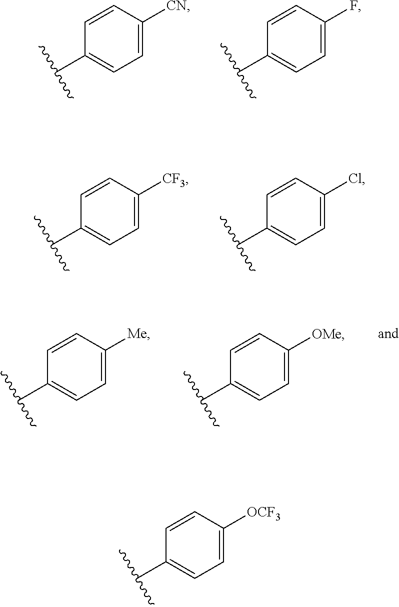

In some embodiments, R² is selected from:

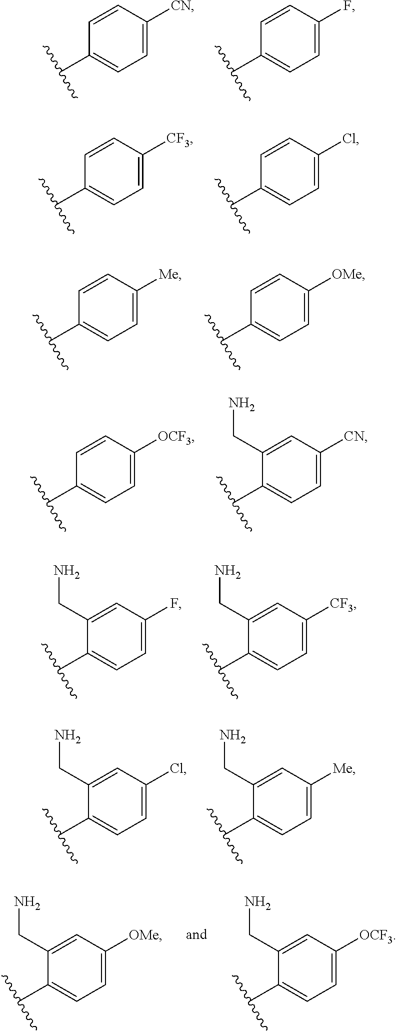

In some embodiments, R¹ is optionally substituted heterocycloalkyl. In some embodiments, R¹ is selected from:

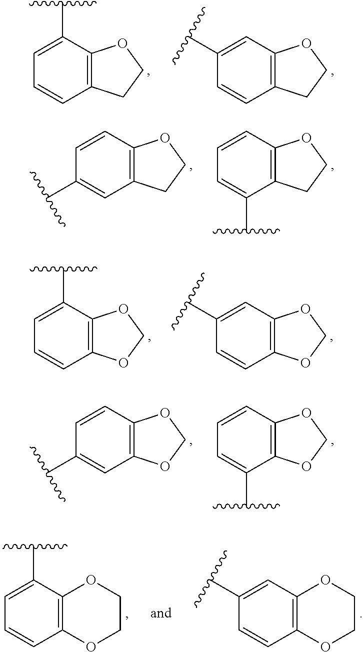

In some embodiments, R² is optionally substituted heterocycloalkyl. In some embodiments, R² is selected from:

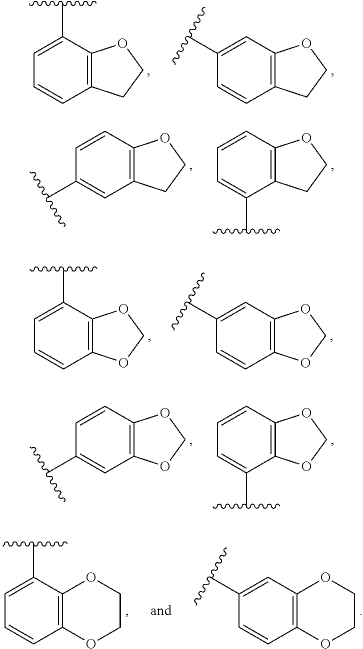

In some embodiments, the compound of Formula (IVa) is selected from:

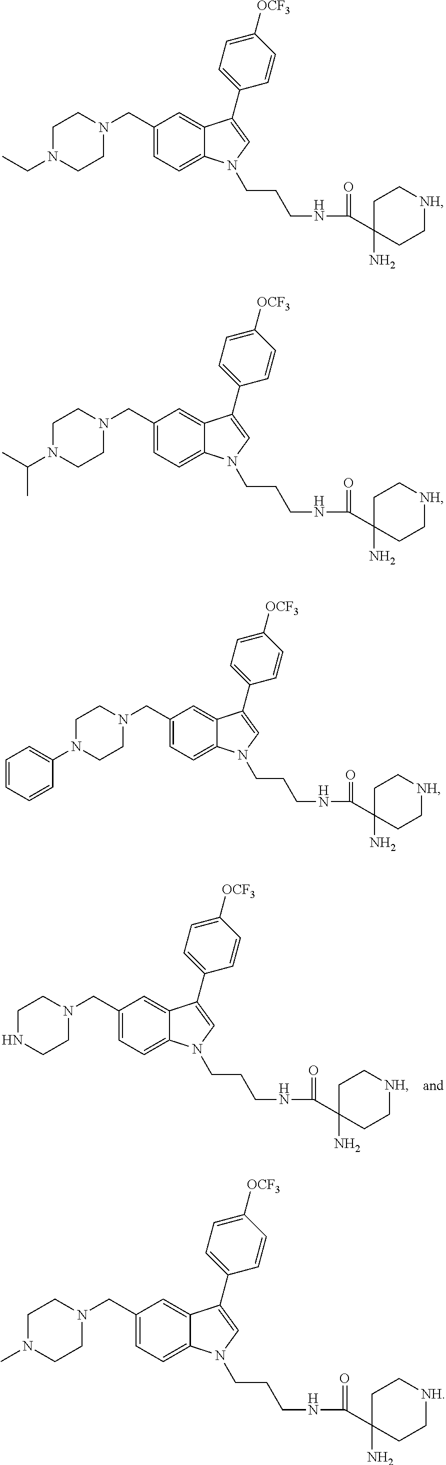 [SMILES: CCN1CCN(Cc2ccc3c(c2)c(-c2ccc(OC(F)(F)F)cc2)cn3CCCNC(=O)C2(N)CCNCC2)CC1 ; CC(C)N1CCN(Cc2ccc3c(c2)c(-c2ccc(OC(F)(F)F)cc2)cn3CCCNC(=O)C2(N)CCNCC2)CC1 ; NC1(C(=O)NCCCn2cc(-c3ccc(OC(F)(F)F)cc3)c3cc(CN4CCN(c5ccccc5)CC4)ccc32)CCNCC1 ; NC1(C(=O)NCCCn2cc(-c3ccc(OC(F)(F)F)cc3)c3cc(CN4CCNCC4)ccc32)CCNCC1 ; CN1CCN(Cc2ccc3c(c2)c(-c2ccc(OC(F)(F)F)cc2)cn3CCCNC(=O)C2(N)CCNCC2)CC1]

Also provided herein in another aspect is a compound of Formula (Va), or a pharmaceutically acceptable salt, or solvate thereof:

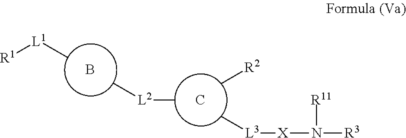

wherein,

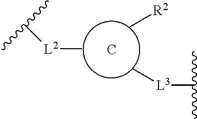

is a bicyclic heteroaryl that is selected from the following structures:

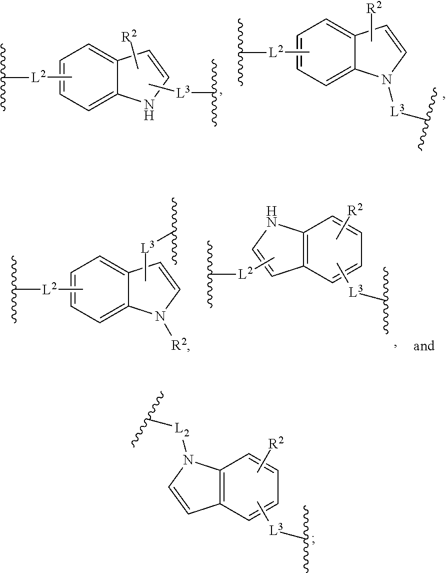

L¹ and L² are each independently absent, an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N with the proviso that Ring B is not:

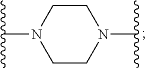 [SMILES: *N1CCN(*)CC1]

wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted phenylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
R³ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, —C₁-C₄alkylene-(optionally substituted heteroaryl), —CH₂C(═O)R¹⁵, —C(═O)R¹⁵, or —CO₂R¹⁶;
R⁴ is hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹⁵ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁶ is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl.

In some embodiments,

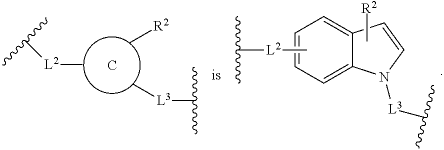

In some embodiments,

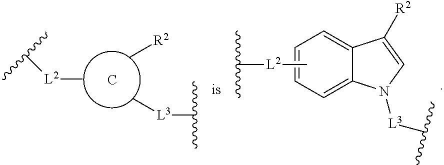

In some embodiments, the compound has the structure of Formula (Vb), or a pharmaceutically acceptable salt, or solvate thereof:

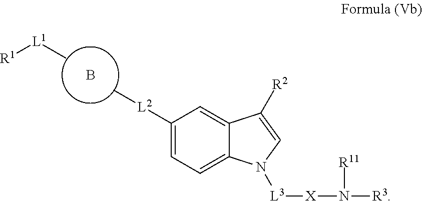

In some embodiments, the compound has the structure of Formula (Vc), or a pharmaceutically acceptable salt, or solvate thereof:

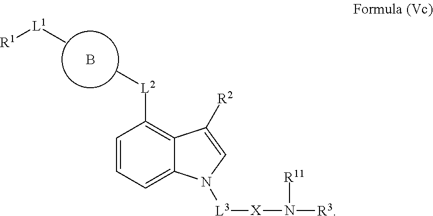

In some embodiments,

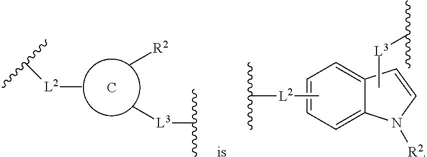

In some embodiments,

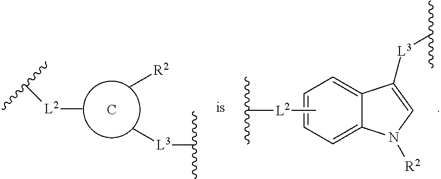

In some embodiments, the compound has the structure of Formula (Vd), or a pharmaceutically acceptable salt, or solvate thereof:

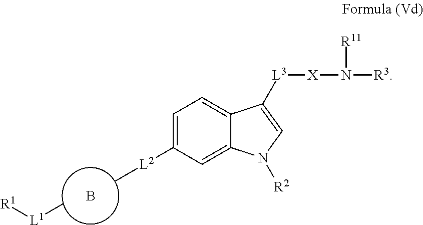

In some embodiments, the compound has the structure of Formula (Ve), or a pharmaceutically acceptable salt, or solvate thereof:

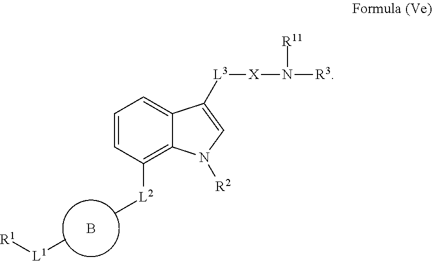

In some embodiments,

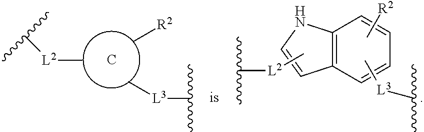

In some embodiments,

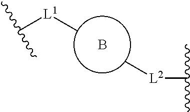

is selected from the following:

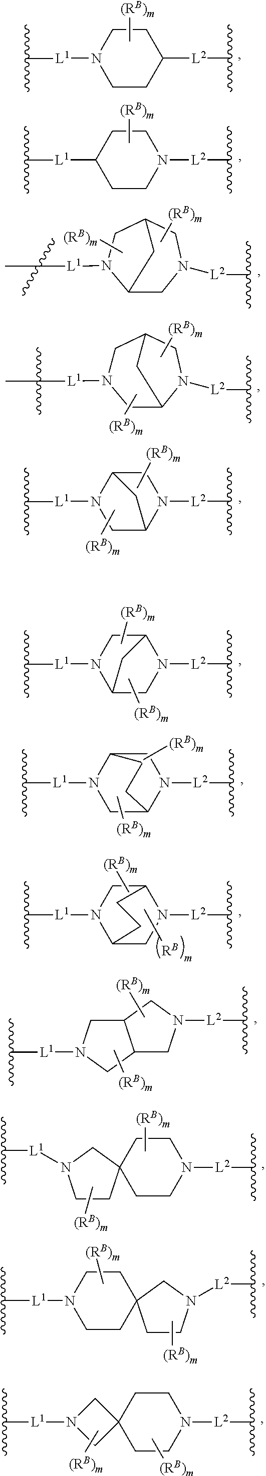

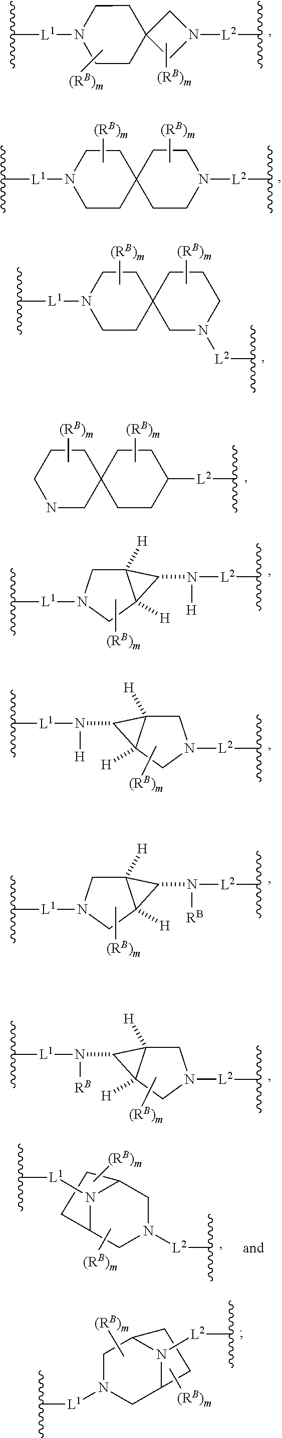

and each m is independently 0, 1, 2, 3, or 4.

In some embodiments,

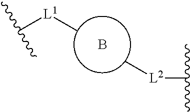

is selected from the following:

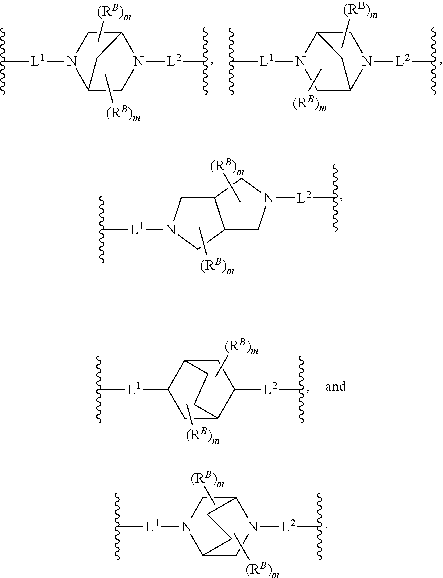

In some embodiments, L¹ is —CH₂—, C(═O), O, S, S(═O), S(═O)₂, or NR⁴. In some embodiments, L¹ is —CH₂—. In some embodiments, L² is —CH₂—, C(═O), O, S, S(═O), S(═O)₂, or NR⁴. In some embodiments, L² is —CH₂—. In some embodiments, L³ is absent, —CH₂—, —CH₂—CH₂—, or —CH₂—CH₂—CH₂—. In some embodiments, L³ is —CH₂—CH₂—.

In some embodiments, L³ is:

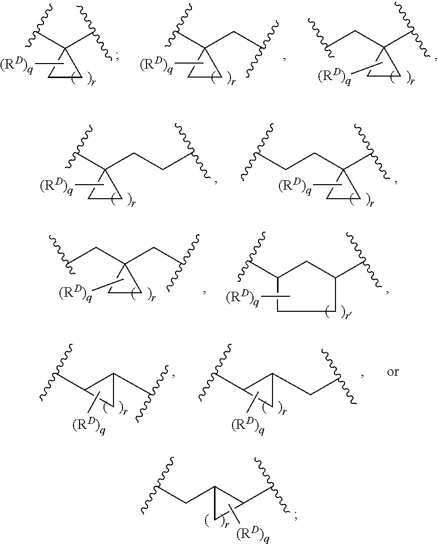

each q is independently 0, 1, 2, 3, or 4;

r is 1, 2, 3, or 4; and

r′ is 1 or 2.

In some embodiments, X is —CH₂— or C(═O). In some embodiments, X is —CH₂—.

In some embodiments, X is:

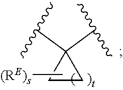

each s is independently 0, 1, 2, 3, or 4; and

t is 1, 2, 3, or 4.

In some embodiments, X is:

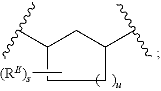

and

each s is independently 0, 1, 2, 3, or 4; and

u is 0, 1, or 2.

In some embodiments, L³-X is —CH₂—CH₂—CH₂—. In some embodiments, R¹¹ and R³ are each independently hydrogen, optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹¹ and R³ are each hydrogen. In some embodiments, R¹¹ and R³ are each optionally substituted C₁-C₆alkyl. In some embodiments, R¹¹ and R³ are each optionally substituted C₁-C₆heteroalkyl. In some embodiments, R¹⁵ and R¹⁶ are each independently optionally substituted C₁-C₆alkyl or optionally substituted C₁-C₆heteroalkyl. In some embodiments, R¹¹ is hydrogen and R³ is optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹ is an unsubstituted phenyl. In some embodiments, R¹ is a substituted phenyl. In some embodiments, R¹ is selected from:

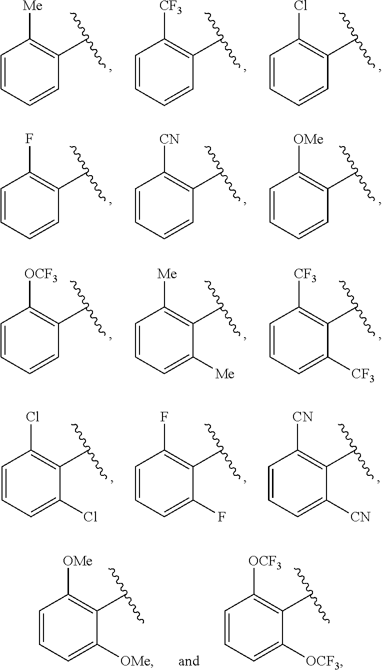

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

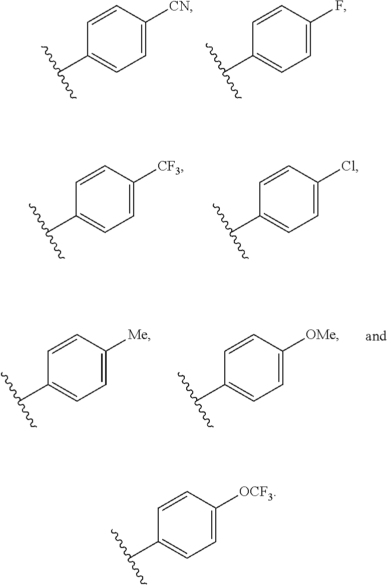

In some embodiments, R² is selected from:

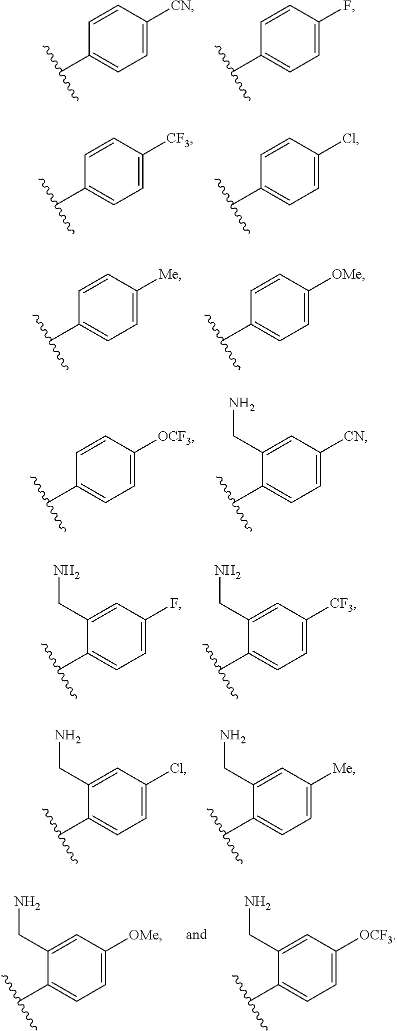

In some embodiments, R¹ is optionally substituted heterocycloalkyl. In some embodiments, R¹ is selected from:

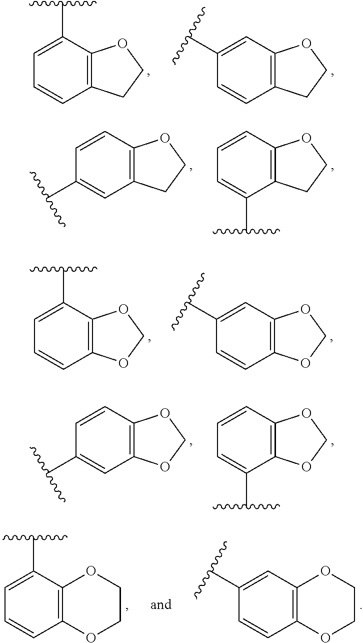

In some embodiments, R² is optionally substituted heterocycloalkyl. In some embodiments, R² is selected from:

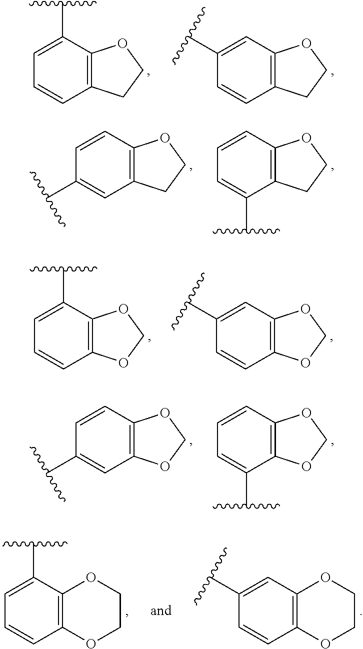

In some embodiments, the compound has the following structure of Formula (Vf), or a pharmaceutically acceptable salt, or solvate thereof:

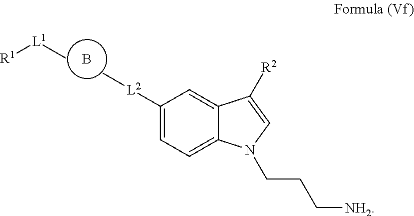

In some embodiments, the compound has the following structure of Formula (Vg), or a pharmaceutically acceptable salt, or solvate thereof:

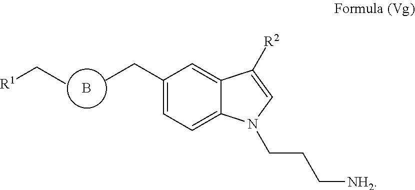

wherein R¹ and R² are each independently optionally substituted aryl; and

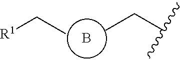

is selected from

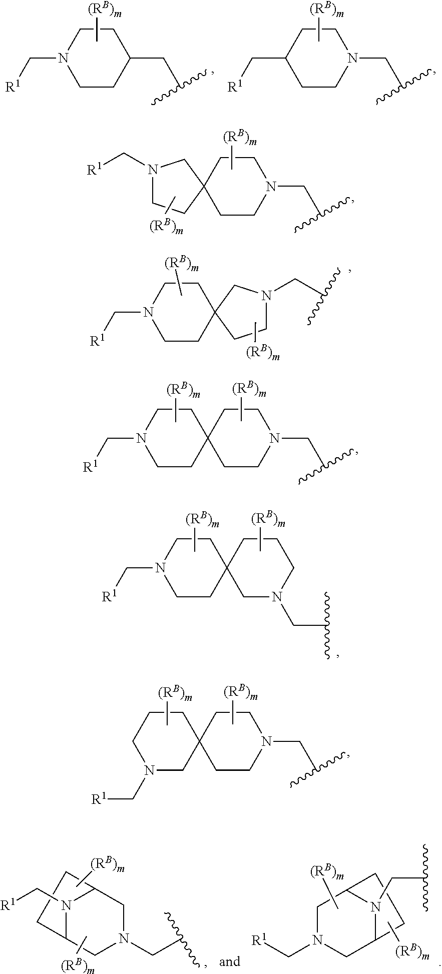

In some embodiments, the compound of Formula (Va) is selected from:

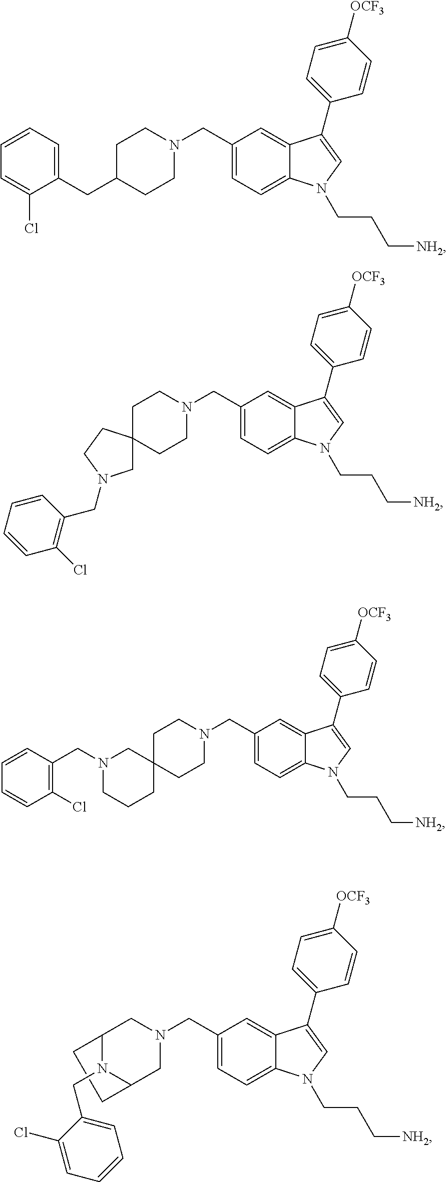

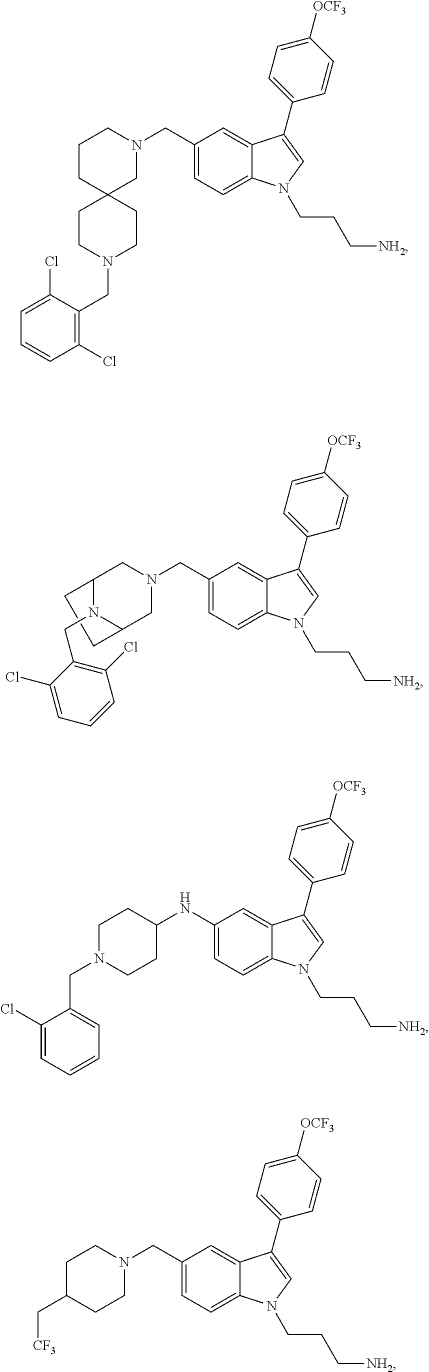 [SMILES: NCCCn1cc(-c2ccc(OC(F)(F)F)cc2)c2cc(CN3CCCC4(CCN(Cc5c(Cl)cccc5Cl)CC4)C3)ccc21 ; NCCCn1cc(-c2ccc(OC(F)(F)F)cc2)c2cc(CN3CC4CCC(C3)N4Cc3c(Cl)cccc3Cl)ccc21 ; NCCCn1cc(-c2ccc(OC(F)(F)F)cc2)c2cc(NC3CCN(Cc4ccccc4Cl)CC3)ccc21 ; NCCCn1cc(-c2ccc(OC(F)(F)F)cc2)c2cc(CN3CCC(CC(F)(F)F)CC3)ccc21]

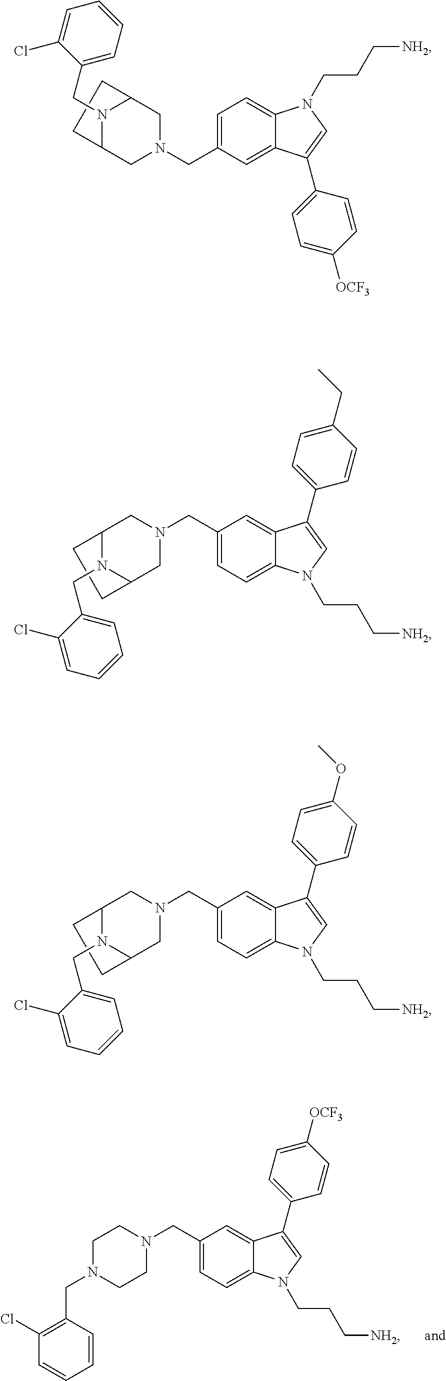 [SMILES: NCCCn1cc(-c2ccc(OC(F)(F)F)cc2)c2cc(CN3CC4CCC(C3)N4Cc3ccccc3Cl)ccc21 ; CCc1ccc(-c2cn(CCCN)c3ccc(CN4CC5CCC(C4)N5Cc4ccccc4Cl)cc23)cc1 ; COc1ccc(-c2cn(CCCN)c3ccc(CN4CC5CCC(C4)N5Cc4ccccc4Cl)cc23)cc1 ; NCCCn1cc(-c2ccc(OC(F)(F)F)cc2)c2cc(CN3CCN(Cc4ccccc4Cl)CC3)ccc21 ; "and"]

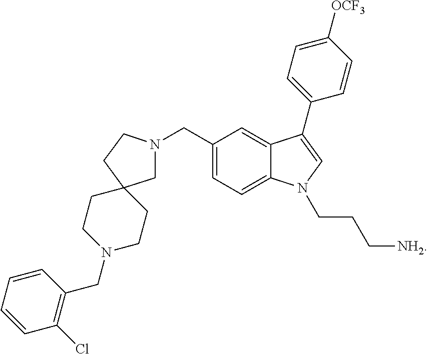 [SMILES: NCCCn1cc(-c2ccc(OC(F)(F)F)cc2)c2cc(CN3CCC4(CCN(Cc5ccccc5Cl)CC4)C3)ccc21]

In some embodiments, the compound of Formula (Va) is selected from:

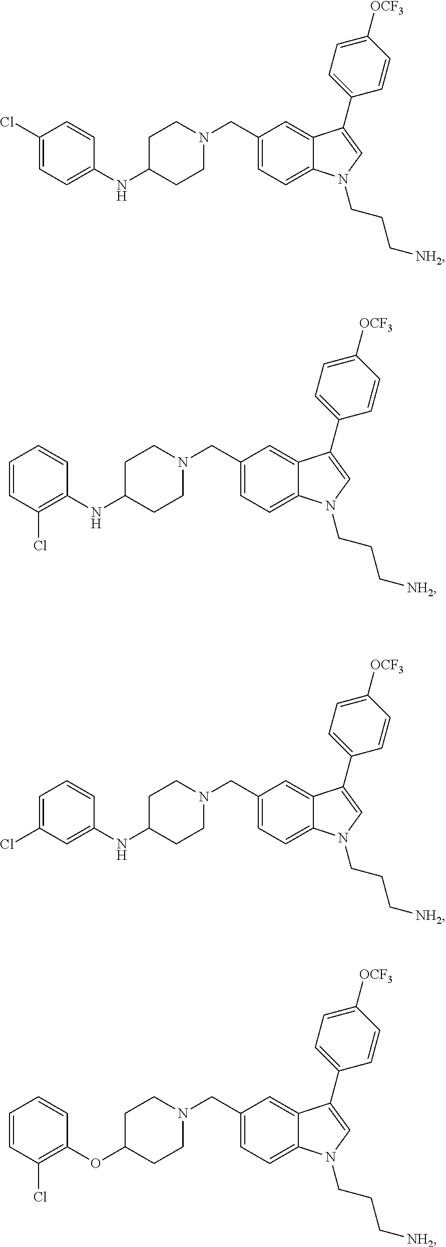 [SMILES: NCCCn1cc(-c2ccc(OC(F)(F)F)cc2)c2cc(CN3CCC(Nc4ccc(Cl)cc4)CC3)ccc21 ; NCCCn1cc(-c2ccc(OC(F)(F)F)cc2)c2cc(CN3CCC(Nc4ccccc4Cl)CC3)ccc21 ; NCCCn1cc(-c2ccc(OC(F)(F)F)cc2)c2cc(CN3CCC(Nc4cccc(Cl)c4)CC3)ccc21 ; NCCCn1cc(-c2ccc(OC(F)(F)F)cc2)c2cc(CN3CCC(Oc4ccccc4Cl)CC3)ccc21]

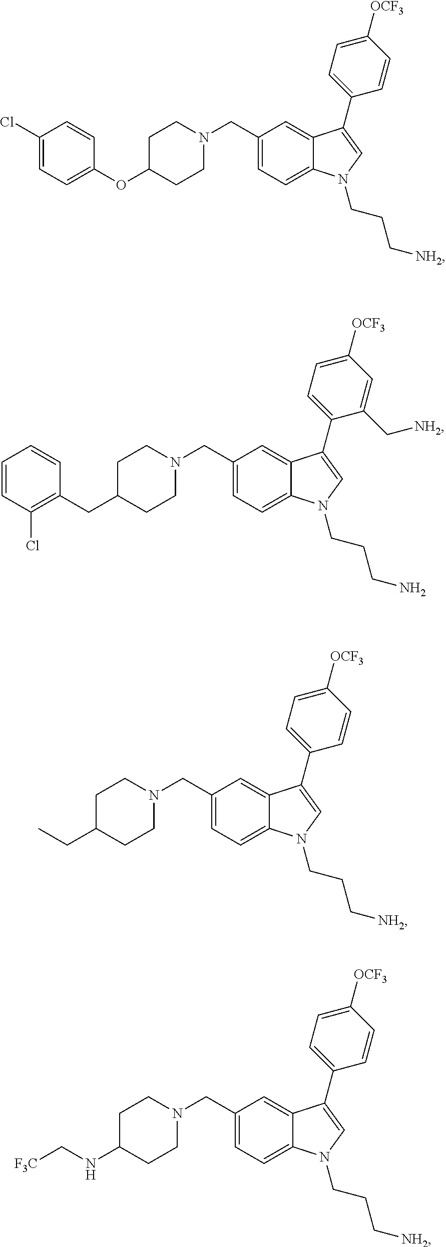 [SMILES: NCCCn1cc(-c2ccc(OC(F)(F)F)cc2)c2cc(CN3CCC(Oc4ccc(Cl)cc4)CC3)ccc21 ; NCCCn1cc(-c2ccc(OC(F)(F)F)cc2CN)c2cc(CN3CCC(Cc4ccccc4Cl)CC3)ccc21 ; CCC1CCN(Cc2ccc3c(c2)c(-c2ccc(OC(F)(F)F)cc2)cn3CCCN)CC1 ; NCCCn1cc(-c2ccc(OC(F)(F)F)cc2)c2cc(CN3CCC(NCC(F)(F)F)CC3)ccc21]

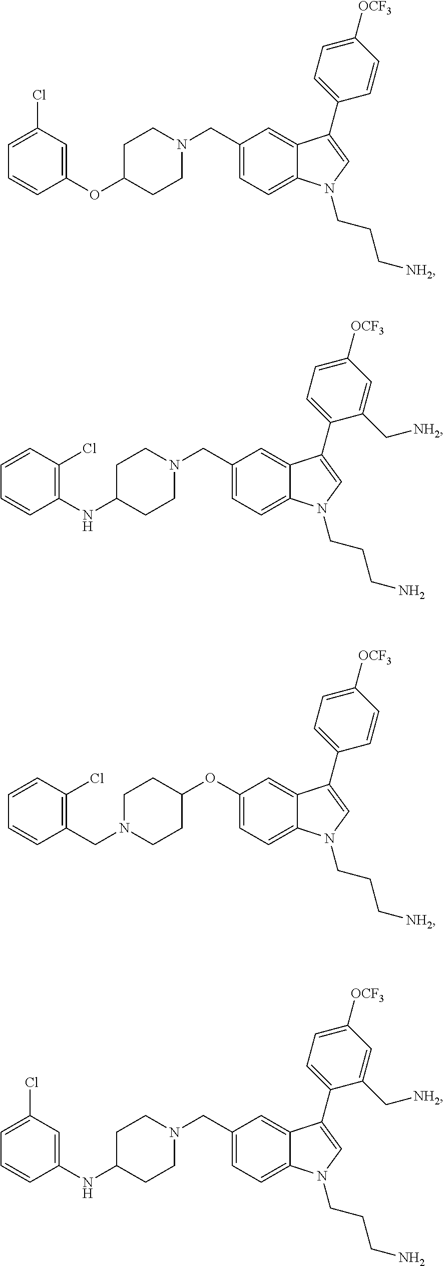 [SMILES: NCCCn1cc(-c2ccc(OC(F)(F)F)cc2)c2cc(CN3CCC(Oc4cccc(Cl)c4)CC3)ccc21 ; NCCCn1cc(-c2ccc(OC(F)(F)F)cc2CN)c2cc(CN3CCC(Nc4ccccc4Cl)CC3)ccc21 ; NCCCn1cc(-c2ccc(OC(F)(F)F)cc2)c2cc(OC3CCN(Cc4ccccc4Cl)CC3)ccc21 ; NCCCn1cc(-c2ccc(OC(F)(F)F)cc2CN)c2cc(CN3CCC(Nc4cccc(Cl)c4)CC3)ccc21]

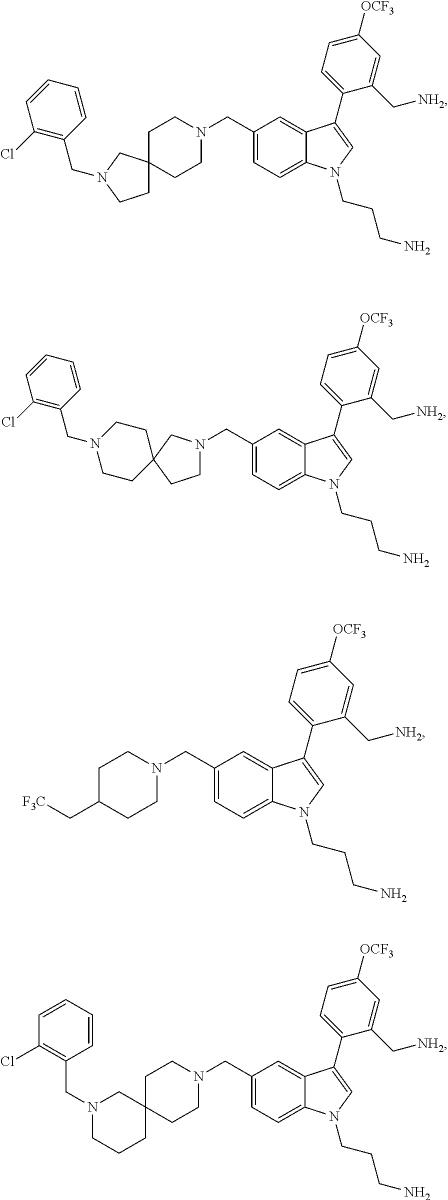 [SMILES: NCCCn1cc(-c2ccc(OC(F)(F)F)cc2CN)c2cc(CN3CCC4(CC3)CCN(Cc3ccccc3Cl)C4)ccc21 ; NCCCn1cc(-c2ccc(OC(F)(F)F)cc2CN)c2cc(CN3CCC4(CCN(Cc5ccccc5Cl)CC4)C3)ccc21 ; NCCCn1cc(-c2ccc(OC(F)(F)F)cc2CN)c2cc(CN3CCC(CC(F)(F)F)CC3)ccc21 ; NCCCn1cc(-c2ccc(OC(F)(F)F)cc2CN)c2cc(CN3CCC4(CCCN(Cc5ccccc5Cl)C4)CC3)ccc21]

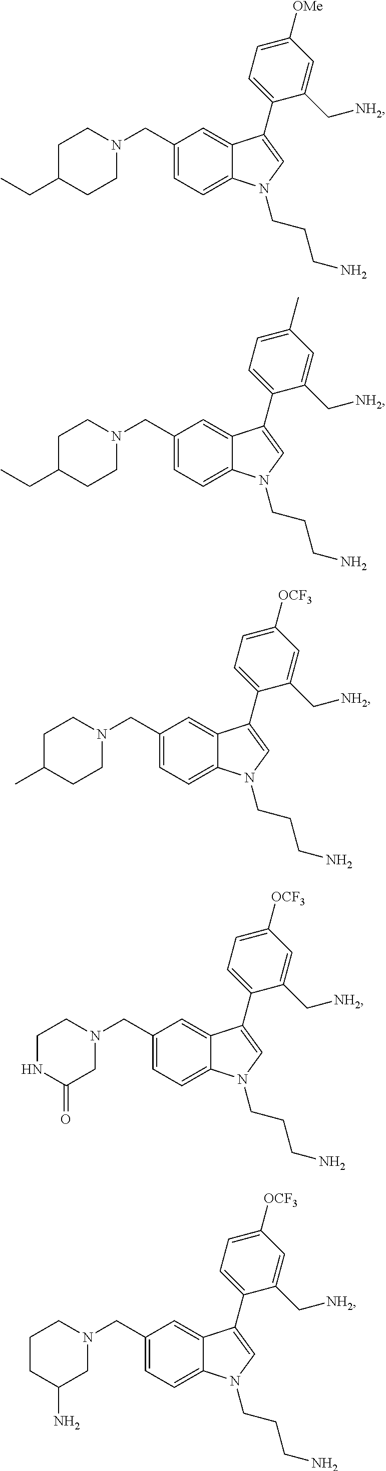

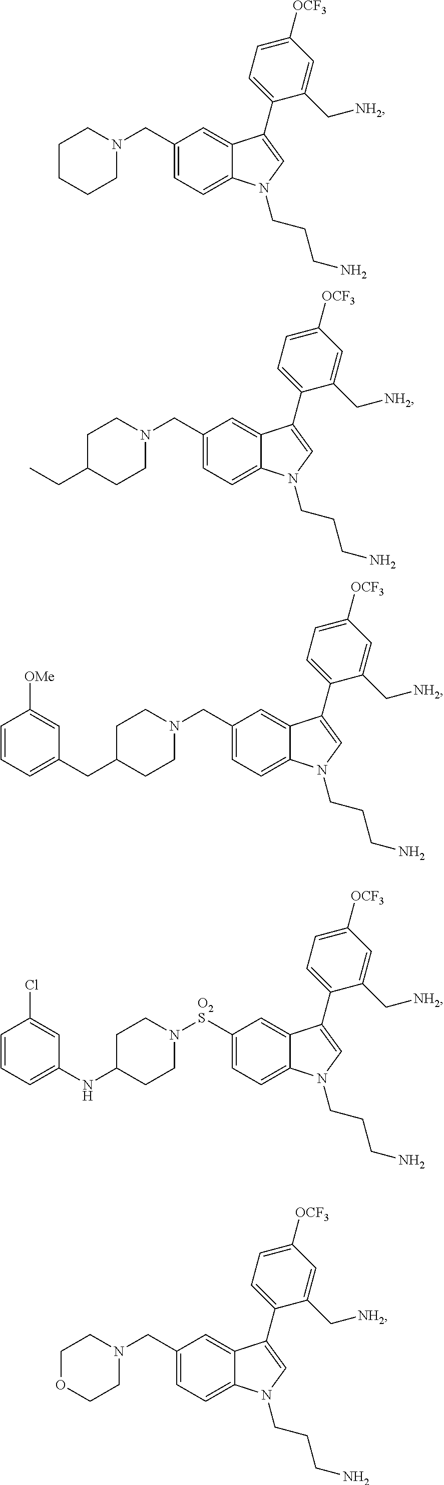

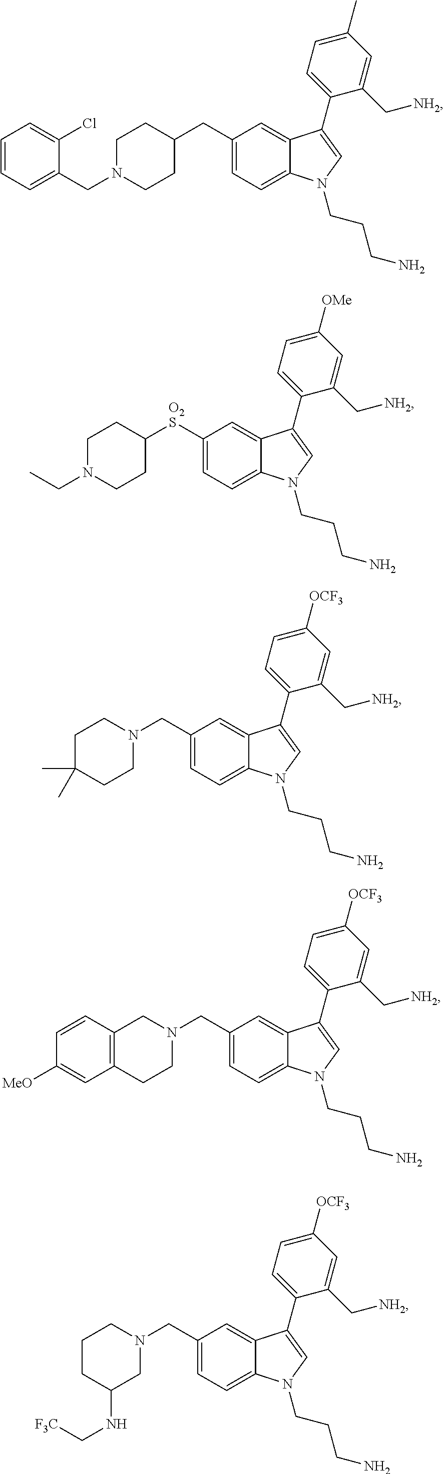

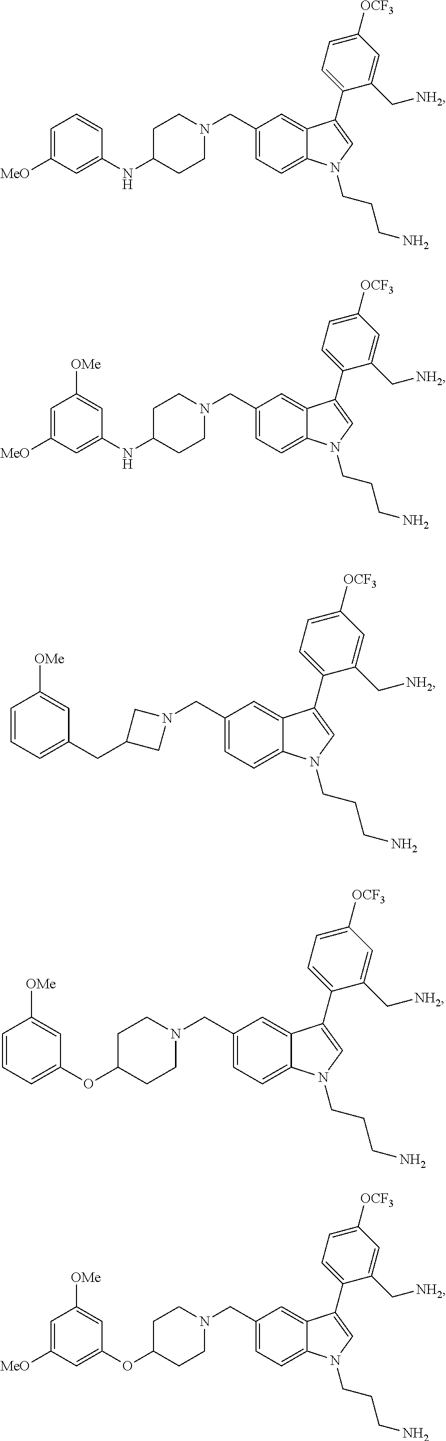 [SMILES: COc1cccc(NC2CCN(Cc3ccc4c(c3)c(-c3ccc(OC(F)(F)F)cc3CN)cn4CCCN)CC2)c1 ; COc1cc(NC2CCN(Cc3ccc4c(c3)c(-c3ccc(OC(F)(F)F)cc3CN)cn4CCCN)CC2)cc(OC)c1 ; COc1cccc(CC2CN(Cc3ccc4c(c3)c(-c3ccc(OC(F)(F)F)cc3CN)cn4CCCN)C2)c1 ; COc1cccc(OC2CCN(Cc3ccc4c(c3)c(-c3ccc(OC(F)(F)F)cc3CN)cn4CCCN)CC2)c1 ; COc1cc(OC)cc(OC2CCN(Cc3ccc4c(c3)c(-c3ccc(OC(F)(F)F)cc3CN)cn4CCCN)CC2)c1]

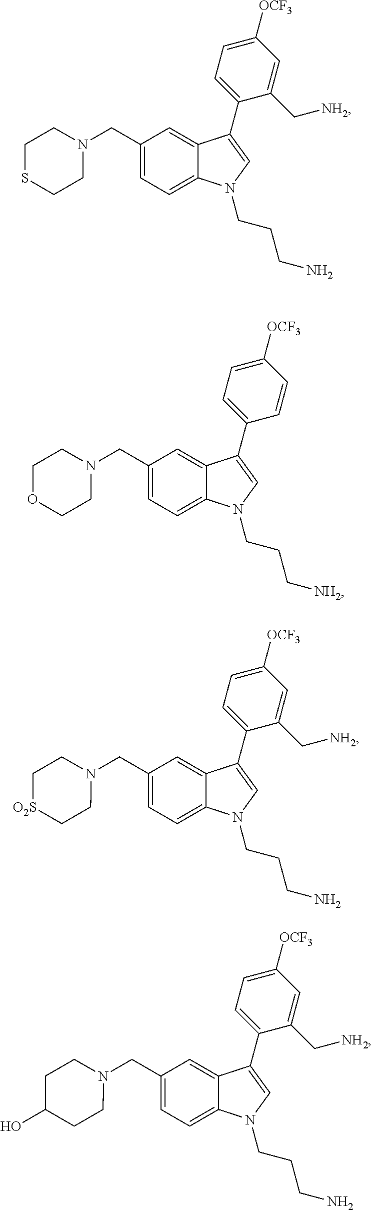

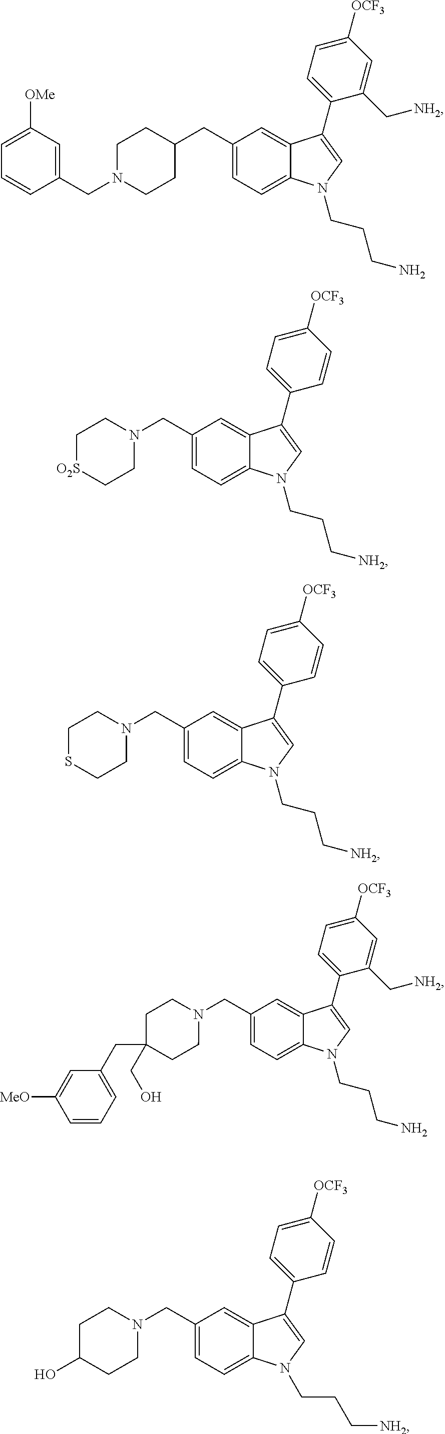

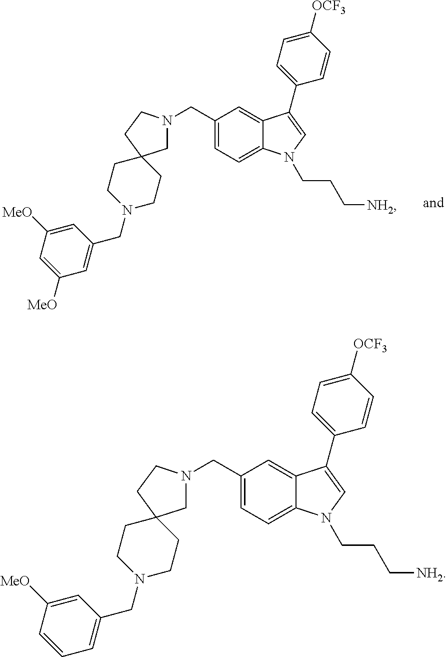 [SMILES: COc1cc(CN2CCC3(CC2)CCN(Cc2ccc4c(c2)c(-c2ccc(OC(F)(F)F)cc2)cn4CCCN)C3)cc(OC)c1 ; "and" ; COc1cccc(CN2CCC3(CC2)CCN(Cc2ccc4c(c2)c(-c2ccc(OC(F)(F)F)cc2)cn4CCCN)C3)c1]

In another aspect provided herein is a compound of Formula (VIa), or a pharmaceutically acceptable salt, or solvate thereof:

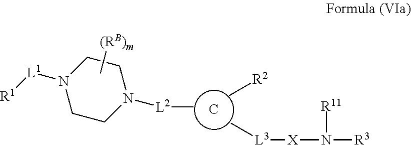

wherein,

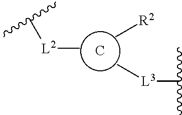

is a bicyclic heteroaryl that is selected from the following structures:

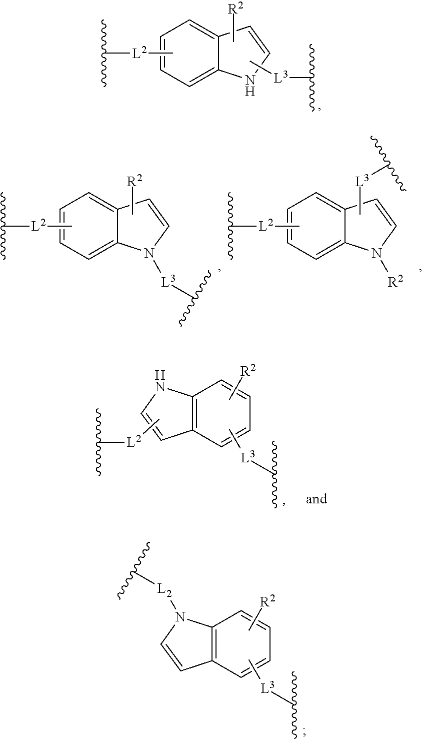

L¹ and L² are each independently absent, an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
each R^{B} is independently optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, a substituted C₁-C₆alkylene, an optionally substituted phenylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
R³ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, —C₁-C₄alkylene-(optionally substituted heteroaryl), —CH₂C(═O)R¹⁵, —C(═O)R¹⁵, or —CO₂R¹⁶;
R⁴ is hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹⁵ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁶ is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each m is independently 0, 1, 2, 3, or 4.

In some embodiments,

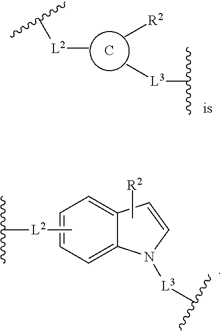

In some embodiments,

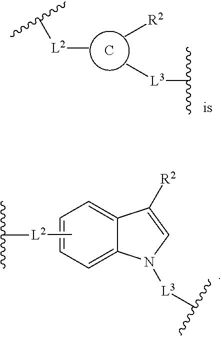

In some embodiments, the compound has the following structure of Formula (VIb), or a pharmaceutically acceptable salt, or solvate thereof:

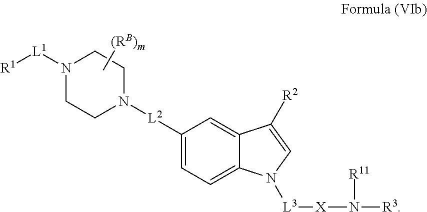

In some embodiments, the compound has the following structure of Formula (VIc), or a pharmaceutically acceptable salt, or solvate thereof:

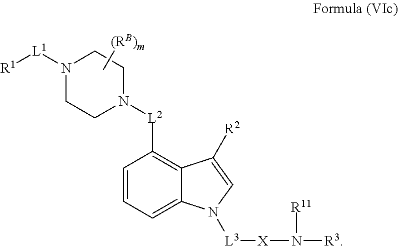

In some embodiments,

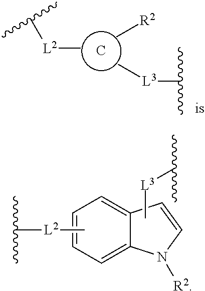

In some embodiments,

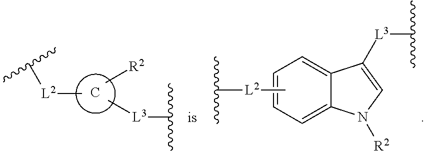

In some embodiments, the compound has the following structure of Formula (VId), or a pharmaceutically acceptable salt, or solvate thereof:

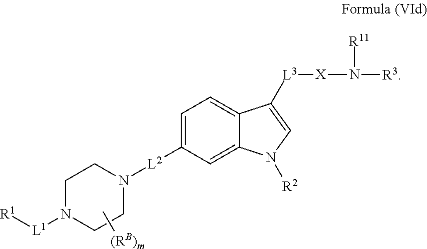

In some embodiments, the compound has the following structure of Formula (VIe), or a pharmaceutically acceptable salt, or solvate thereof:

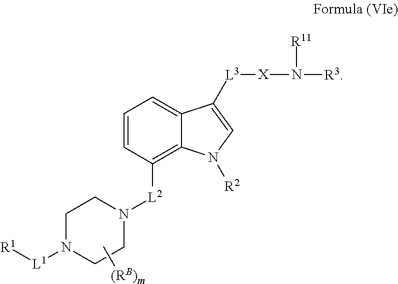

In some embodiments,

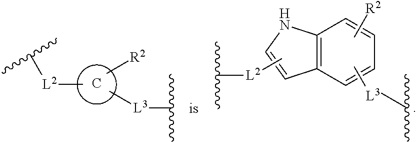

In some embodiments, L¹ is —CH₂—, C(═O), O, S, S(═O), S(═O)₂, or NR⁴. In some embodiments, L¹ is —CH₂—. In some embodiments, L² is —CH₂—, C(═O), O, S, S(═O), S(═O)₂, or NR⁴. In some embodiments, L² is —CH₂—. In some embodiments, L³ is a substituted C₁-C₅alkylene.

In some embodiments, L³ is:

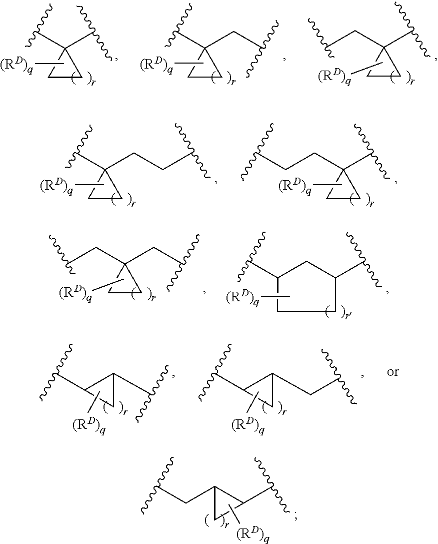

each q is independently 0, 1, 2, 3, or 4;

r is 1, 2, 3, or 4; and

r′ is 1 or 2.

In some embodiments, X is —CH₂— or C(═O). In some embodiments, X is —CH₂—.

In some embodiments, X is:

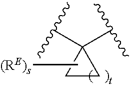

each s is independently 0, 1, 2, 3, or 4; and

t is 1, 2, 3, or 4.

In some embodiments, X is:

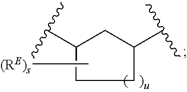

each s is independently 0, 1, 2, 3, or 4; and

u is 0, 1, or 2.

In some embodiments, R¹¹ and R³ are each independently hydrogen, optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹¹ and R³ are each hydrogen. In some embodiments, R¹¹ and R³ are each optionally substituted C₁-C₆alkyl. In some embodiments, R¹¹ and R³ are each optionally substituted C₁-C₆heteroalkyl. In some embodiments, R¹⁵ and R¹⁶ are each independently optionally substituted C₁-C₆alkyl or optionally substituted C₁-C₆heteroalkyl. In some embodiments, R¹¹ is hydrogen and R³ is optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹ is an unsubstituted phenyl. In some embodiments, R¹ is a substituted phenyl. In some embodiments, R¹ is selected from:

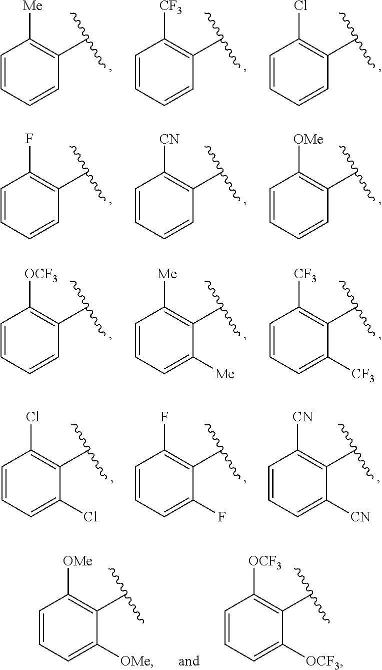

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

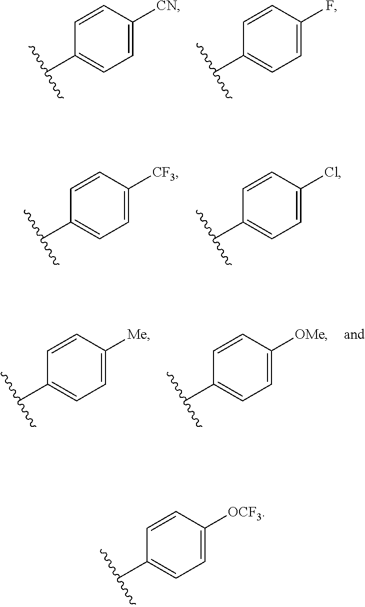

In some embodiments, R² is selected from:

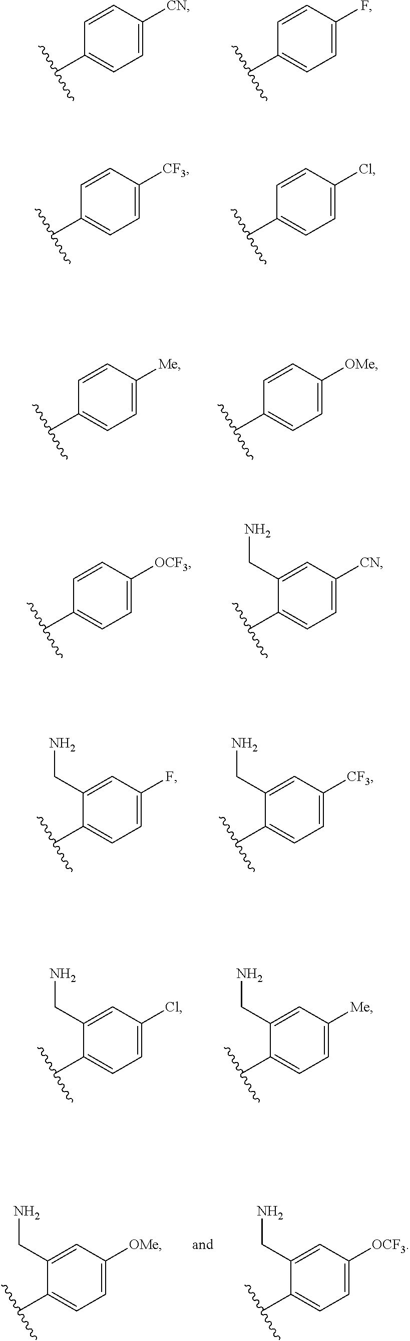

In some embodiments, R¹ is optionally substituted heterocycloalkyl. In some embodiments, R¹ is selected from:

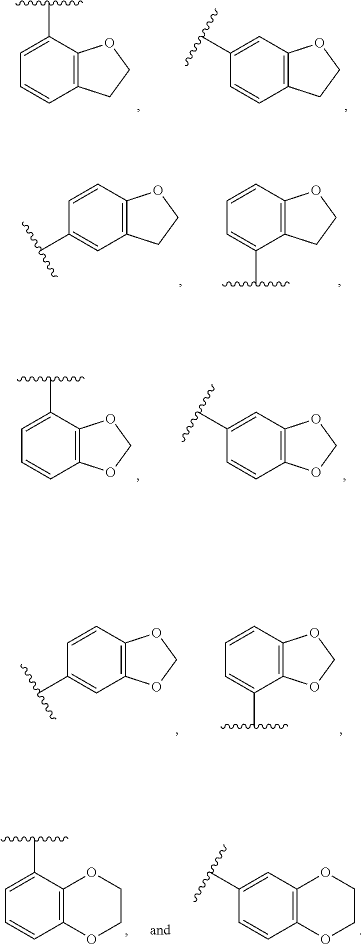

In some embodiments, R² is optionally substituted heterocycloalkyl. In some embodiments, R² is selected from:

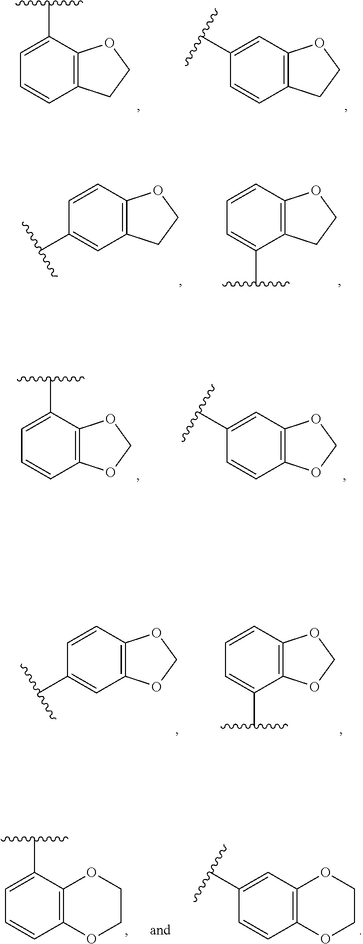

In some embodiments, the compound of Formula (VIa) is selected from:

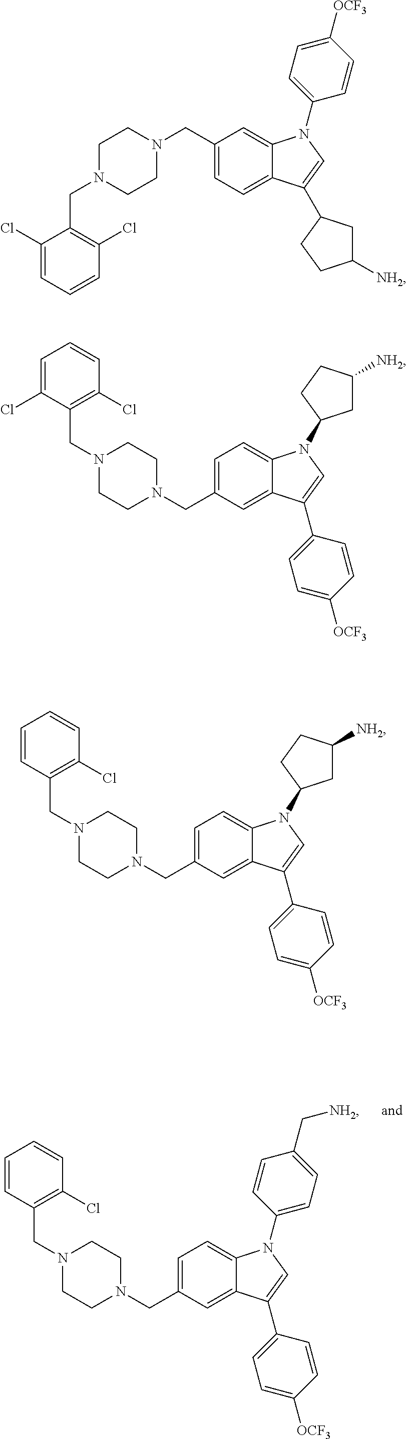 [SMILES: NC1CCC(c2cn(-c3ccc(OC(F)(F)F)cc3)c3cc(CN4CCN(Cc5c(Cl)cccc5Cl)CC4)ccc23)C1 ; N[C@H]1CC[C@H](n2cc(-c3ccc(OC(F)(F)F)cc3)c3cc(CN4CCN(Cc5c(Cl)cccc5Cl)CC4)ccc32)C1 ; N[C@@H]1CC[C@H](n2cc(-c3ccc(OC(F)(F)F)cc3)c3cc(CN4CCN(Cc5ccccc5Cl)CC4)ccc32)C1 ; NCc1ccc(-n2cc(-c3ccc(OC(F)(F)F)cc3)c3cc(CN4CCN(Cc5ccccc5Cl)CC4)ccc32)cc1 ; "and"]

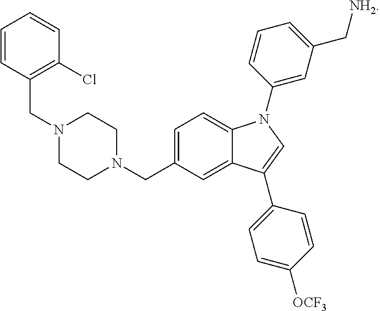 [SMILES: NCc1cccc(-n2cc(-c3ccc(OC(F)(F)F)cc3)c3cc(CN4CCN(Cc5ccccc5Cl)CC4)ccc32)c1]

In some embodiments, the compound of Formula (VIa) is selected from:

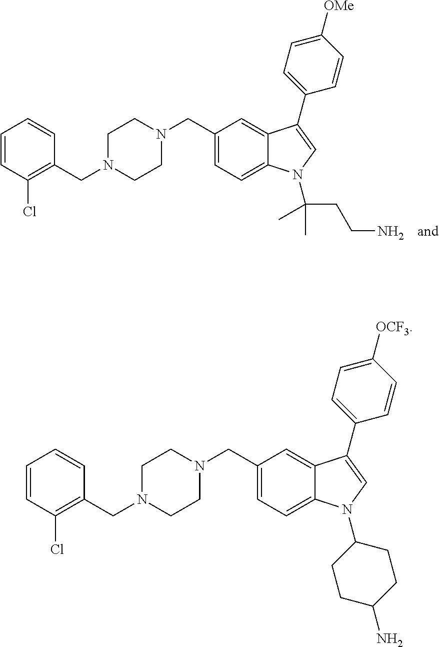 [SMILES: COc1ccc(-c2cn(C(C)(C)CCN)c3ccc(CN4CCN(Cc5ccccc5Cl)CC4)cc23)cc1 ; "and" ; NC1CCC(n2cc(-c3ccc(OC(F)(F)F)cc3)c3cc(CN4CCN(Cc5ccccc5Cl)CC4)ccc32)CC1]

Also provided herein is a compound of Formula (VIIa), or a pharmaceutically acceptable salt, or solvate thereof:

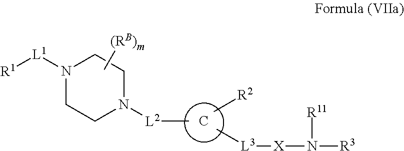

wherein,

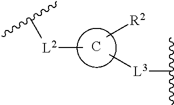

is a bicyclic heteroaryl that is selected from the following structures:

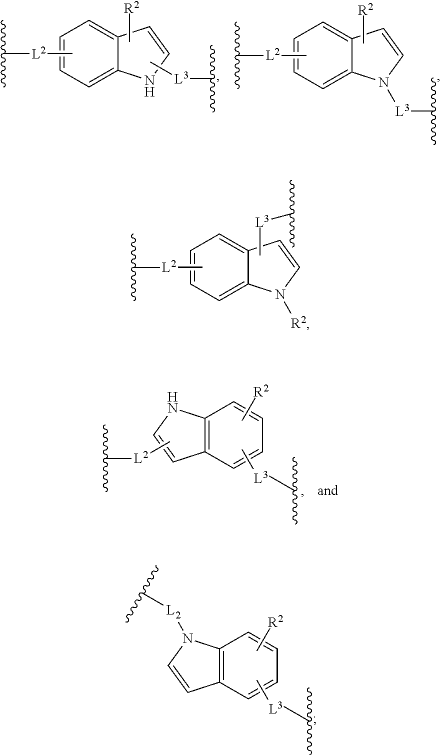

L¹ and L² are each independently absent, an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
each R^{B} is independently optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
L³ is an unsubstituted C₁-C₆alkylene;
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
R³ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, —C₁-C₄alkylene-(optionally substituted heteroaryl), —CH₂C(═O)R¹⁵, —C(═O)R¹⁵, or —CO₂R¹⁶;
R⁴ is hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹⁵ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁶ is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each m is independently 0, 1, 2, 3, or 4; and with the provision that the compound is not

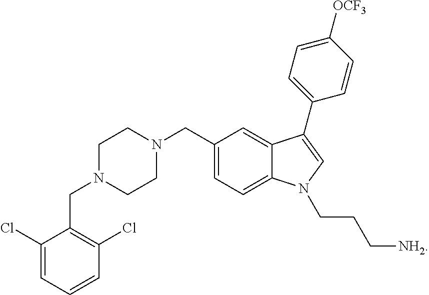 [SMILES: NCCCn1cc(-c2ccc(OC(F)(F)F)cc2)c2cc(CN3CCN(Cc4c(Cl)cccc4Cl)CC3)ccc21]

In some embodiments,

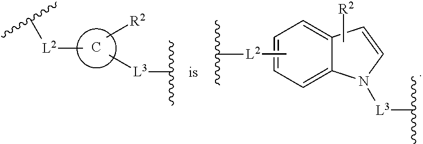

In some embodiments,

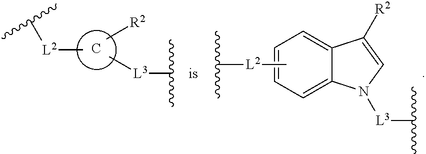

In some embodiments, the compound has the following structure of Formula (VIIb), or a pharmaceutically acceptable salt, or solvate thereof:

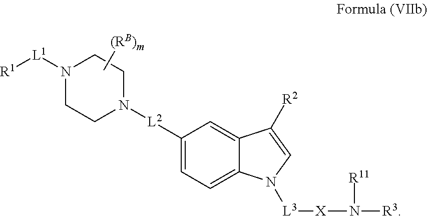

In some embodiments, the compound has the following structure of Formula (VIIc), or a pharmaceutically acceptable salt, or solvate thereof:

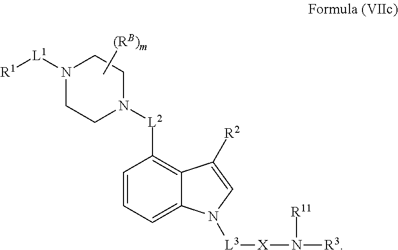

In some embodiments,

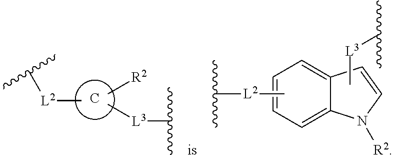

In some embodiments,

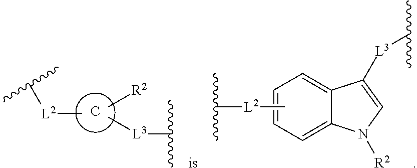

In some embodiments, the compound has the following structure of Formula (VIId), or a pharmaceutically acceptable salt, or solvate thereof:

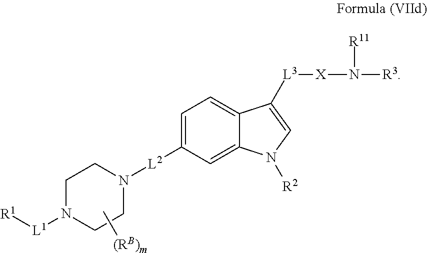

In some embodiments, the compound has the following structure of Formula (VIIe), or a pharmaceutically acceptable salt, or solvate thereof:

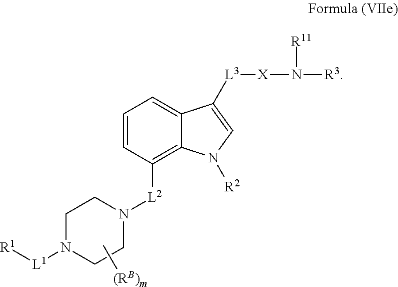

In some embodiments,

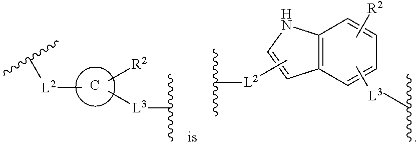

In some embodiments, L¹ is —CH₂—, C(═O), O, S, S(═O), S(═O)₂, or NR⁴. In some embodiments, L¹ is —CH₂—. In some embodiments, L² is —CH₂—, C(═O), O, S, S(═O), S(═O)₂, or NR⁴. In some embodiments, L² is —CH₂—. In some embodiments, L³ is —CH₂—, —CH₂CH₂—, or —CH₂—CH₂—CH₂—. In some embodiments, X is —CH₂— or C(═O). In some embodiments, X is —CH₂—.

In some embodiments, X is:

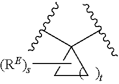

each s is independently 0, 1, 2, 3, or 4; and

t is 1, 2, 3, or 4.

In some embodiments, X is:

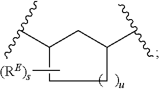

each s is independently 0, 1, 2, 3, or 4; and

u is 0, 1, or 2.

In some embodiments, L³-X is —CH₂—CH₂—CH₂—. In some embodiments, R¹¹ and R³ are each independently hydrogen, optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹¹ and R³ are each hydrogen. In some embodiments, R¹¹ and R³ are each optionally substituted C₁-C₆alkyl. In some embodiments, R¹¹ and R³ are each optionally substituted C₁-C₆heteroalkyl. In some embodiments, R¹⁵ and R¹⁶ are each independently optionally substituted C₁-C₆alkyl or optionally substituted C₁-C₆heteroalkyl. In some embodiments, R¹¹ is hydrogen and R³ is optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹ is an unsubstituted phenyl. In some embodiments, R¹ is a substituted phenyl. In some embodiments, R¹ is selected from:

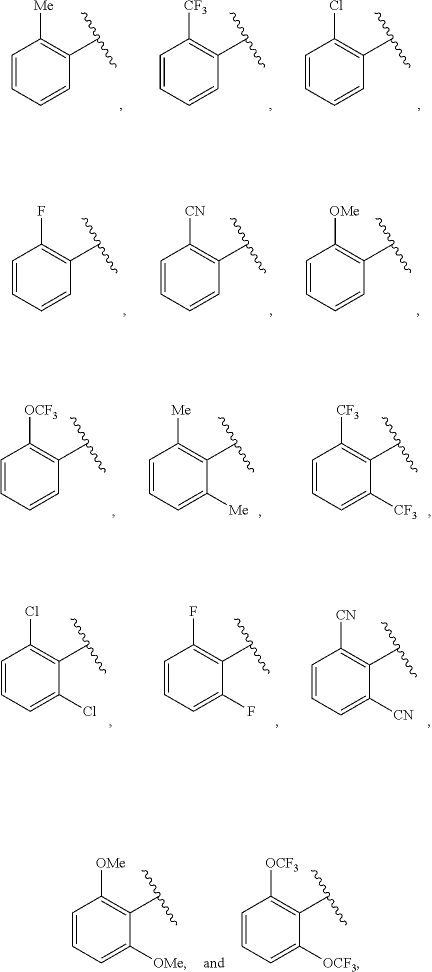

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

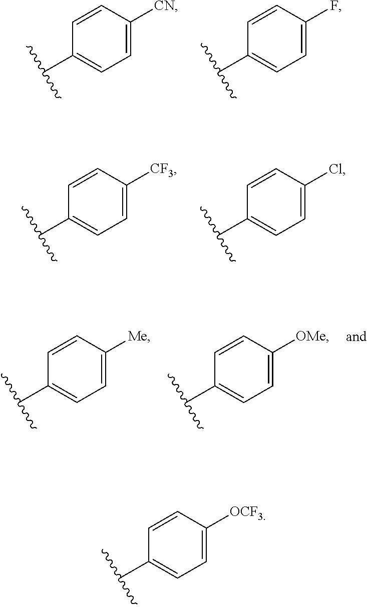

In some embodiments, R² is selected from:

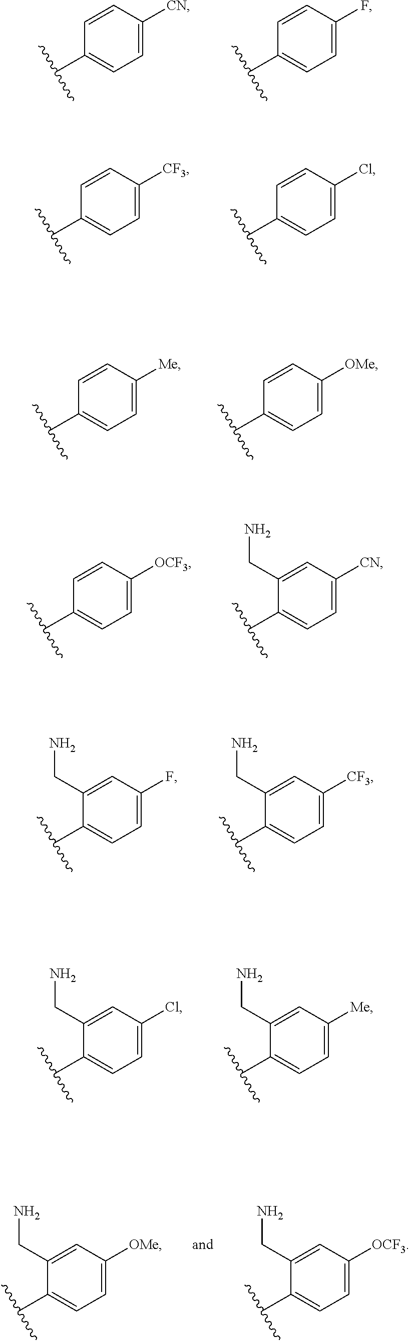

In some embodiments, R¹ is optionally substituted heterocycloalkyl. In some embodiments, R¹ is selected from:

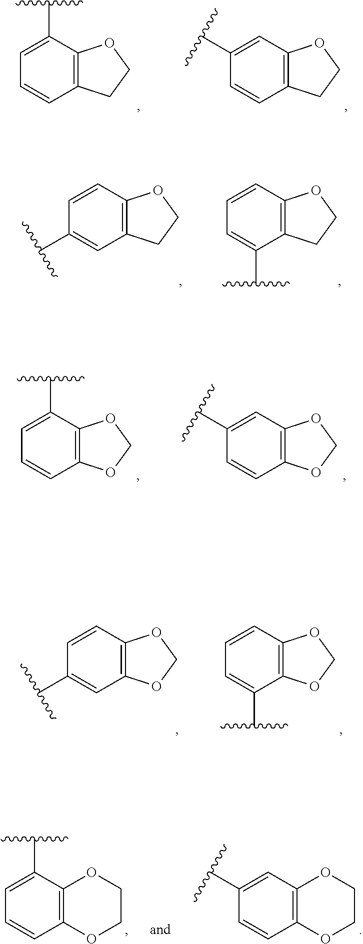

In some embodiments, R² is optionally substituted heterocycloalkyl. In some embodiments, R² is selected from:

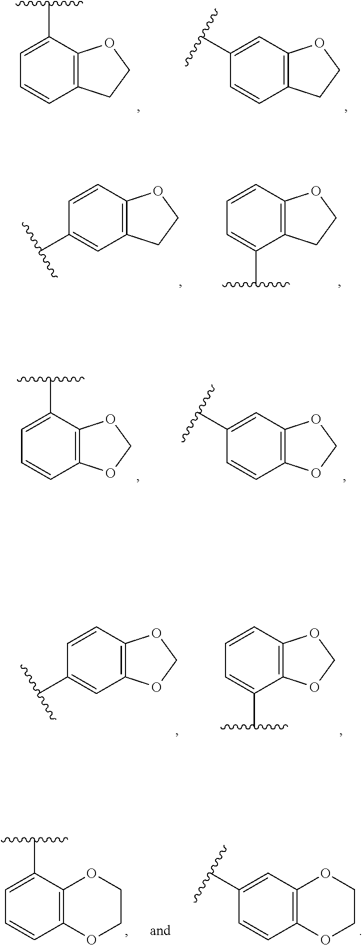

In some embodiments, the compounds has the following structure of Formula (VIIf), or a pharmaceutically acceptable salt, or solvate thereof:

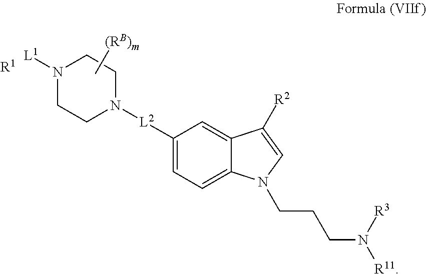

wherein R¹ and R² are each independently optionally substituted aryl.

In some embodiments, the compound has the following structure of Formula (VIIg), or a pharmaceutically acceptable salt, or solvate thereof:

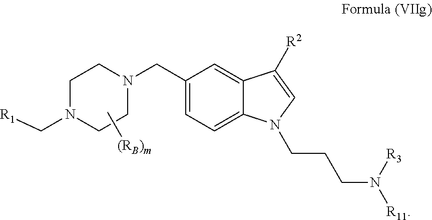

wherein R¹ and R² are each independently optionally substituted aryl.

In some embodiments, the compound of Formula (VIIa) is selected from:

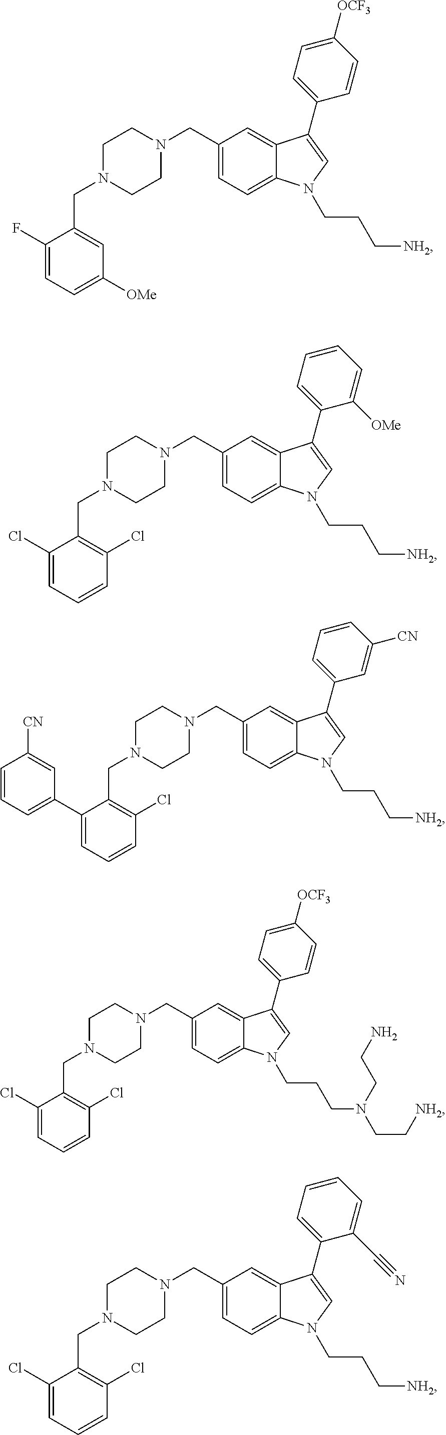 [SMILES: COc1ccc(F)c(CN2CCN(Cc3ccc4c(c3)c(-c3ccc(OC(F)(F)F)cc3)cn4CCCN)CC2)c1 ; COc1ccccc1-c1cn(CCCN)c2ccc(CN3CCN(Cc4c(Cl)cccc4Cl)CC3)cc12 ; N#Cc1cccc(-c2cccc(Cl)c2CN2CCN(Cc3ccc4c(c3)c(-c3cccc(C#N)c3)cn4CCCN)CC2)c1 ; NCCN(CCN)CCCn1cc(-c2ccc(OC(F)(F)F)cc2)c2cc(CN3CCN(Cc4c(Cl)cccc4Cl)CC3)ccc21 ; N#Cc1ccccc1-c1cn(CCCN)c2ccc(CN3CCN(Cc4c(Cl)cccc4Cl)CC3)cc12]

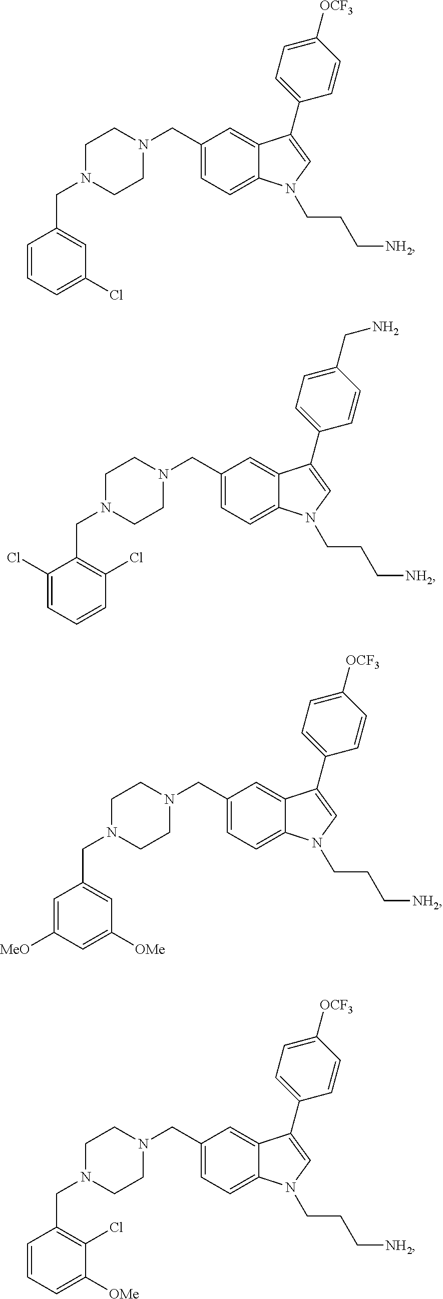 [SMILES: NCCCn1cc(-c2ccc(OC(F)(F)F)cc2)c2cc(CN3CCN(Cc4cccc(Cl)c4)CC3)ccc21 ; NCCCn1cc(-c2ccc(CN)cc2)c2cc(CN3CCN(Cc4c(Cl)cccc4Cl)CC3)ccc21 ; COc1cc(CN2CCN(Cc3ccc4c(c3)c(-c3ccc(OC(F)(F)F)cc3)cn4CCCN)CC2)cc(OC)c1 ; COc1cccc(CN2CCN(Cc3ccc4c(c3)c(-c3ccc(OC(F)(F)F)cc3)cn4CCCN)CC2)c1Cl]

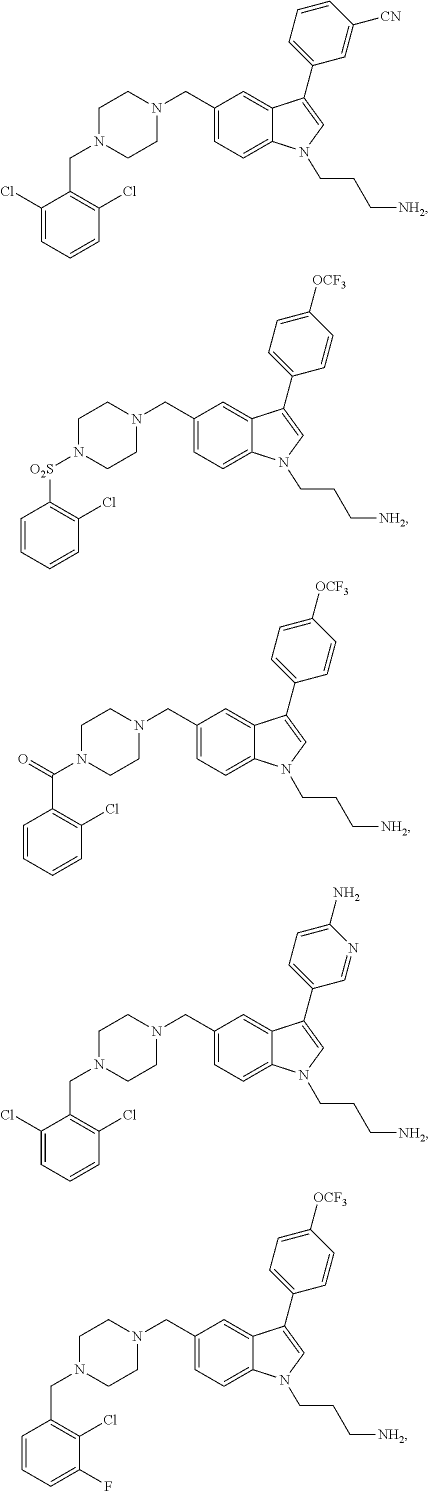 [SMILES: N#Cc1cccc(-c2cn(CCCN)c3ccc(CN4CCN(Cc5c(Cl)cccc5Cl)CC4)cc23)c1 ; NCCCn1cc(-c2ccc(OC(F)(F)F)cc2)c2cc(CN3CCN(S(=O)(=O)c4ccccc4Cl)CC3)ccc21 ; NCCCn1cc(-c2ccc(OC(F)(F)F)cc2)c2cc(CN3CCN(C(=O)c4ccccc4Cl)CC3)ccc21 ; NCCCn1cc(-c2ccc(N)nc2)c2cc(CN3CCN(Cc4c(Cl)cccc4Cl)CC3)ccc21 ; NCCCn1cc(-c2ccc(OC(F)(F)F)cc2)c2cc(CN3CCN(Cc4cccc(F)c4Cl)CC3)ccc21]

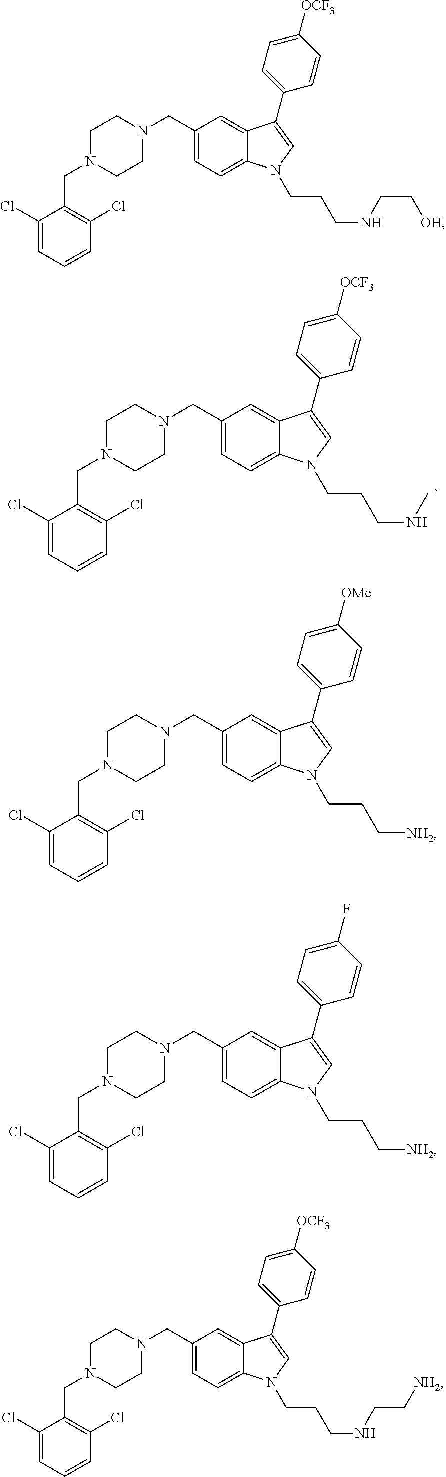 [SMILES: OCCNCCCn1cc(-c2ccc(OC(F)(F)F)cc2)c2cc(CN3CCN(Cc4c(Cl)cccc4Cl)CC3)ccc21 ; CNCCCn1cc(-c2ccc(OC(F)(F)F)cc2)c2cc(CN3CCN(Cc4c(Cl)cccc4Cl)CC3)ccc21 ; COc1ccc(-c2cn(CCCN)c3ccc(CN4CCN(Cc5c(Cl)cccc5Cl)CC4)cc23)cc1 ; NCCCn1cc(-c2ccc(F)cc2)c2cc(CN3CCN(Cc4c(Cl)cccc4Cl)CC3)ccc21 ; NCCNCCCn1cc(-c2ccc(OC(F)(F)F)cc2)c2cc(CN3CCN(Cc4c(Cl)cccc4Cl)CC3)ccc21]

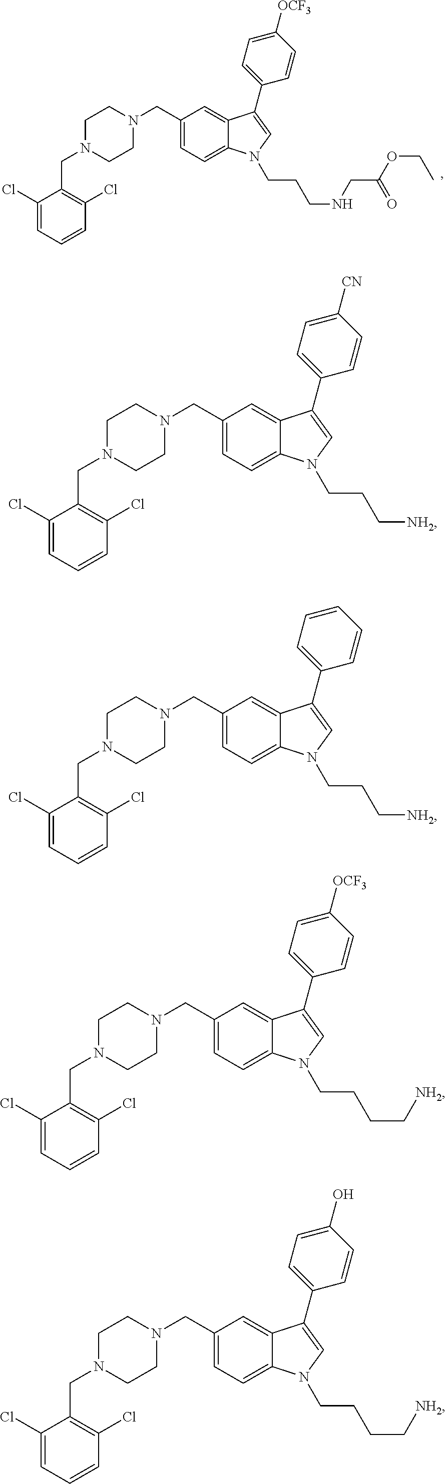 [SMILES: CCOC(=O)CNCCCn1cc(-c2ccc(OC(F)(F)F)cc2)c2cc(CN3CCN(Cc4c(Cl)cccc4Cl)CC3)ccc21 ; N#Cc1ccc(-c2cn(CCCN)c3ccc(CN4CCN(Cc5c(Cl)cccc5Cl)CC4)cc23)cc1 ; NCCCn1cc(-c2ccccc2)c2cc(CN3CCN(Cc4c(Cl)cccc4Cl)CC3)ccc21 ; NCCCCn1cc(-c2ccc(OC(F)(F)F)cc2)c2cc(CN3CCN(Cc4c(Cl)cccc4Cl)CC3)ccc21 ; NCCCCn1cc(-c2ccc(O)cc2)c2cc(CN3CCN(Cc4c(Cl)cccc4Cl)CC3)ccc21]

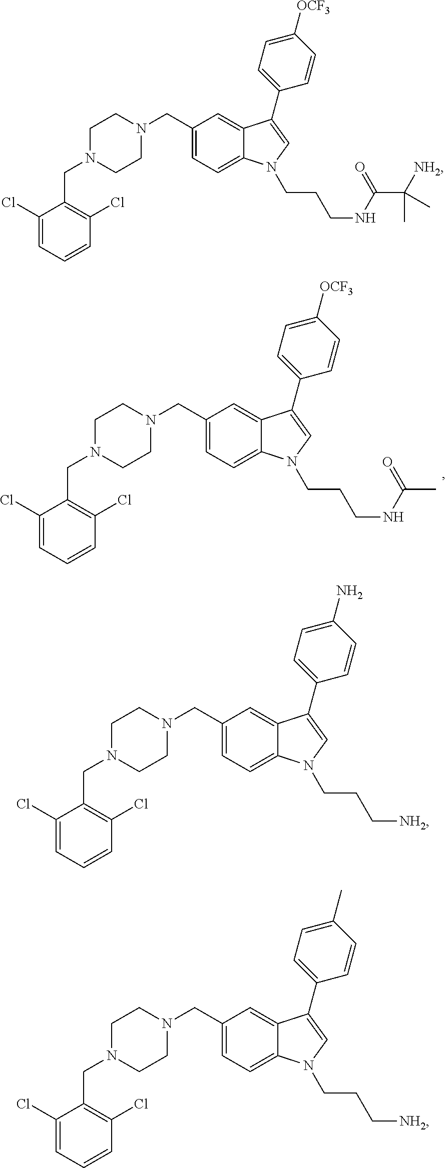 [SMILES: CC(C)(N)C(=O)NCCCn1cc(-c2ccc(OC(F)(F)F)cc2)c2cc(CN3CCN(Cc4c(Cl)cccc4Cl)CC3)ccc21 ; CC(=O)NCCCn1cc(-c2ccc(OC(F)(F)F)cc2)c2cc(CN3CCN(Cc4c(Cl)cccc4Cl)CC3)ccc21 ; NCCCn1cc(-c2ccc(N)cc2)c2cc(CN3CCN(Cc4c(Cl)cccc4Cl)CC3)ccc21 ; Cc1ccc(-c2cn(CCCN)c3ccc(CN4CCN(Cc5c(Cl)cccc5Cl)CC4)cc23)cc1]

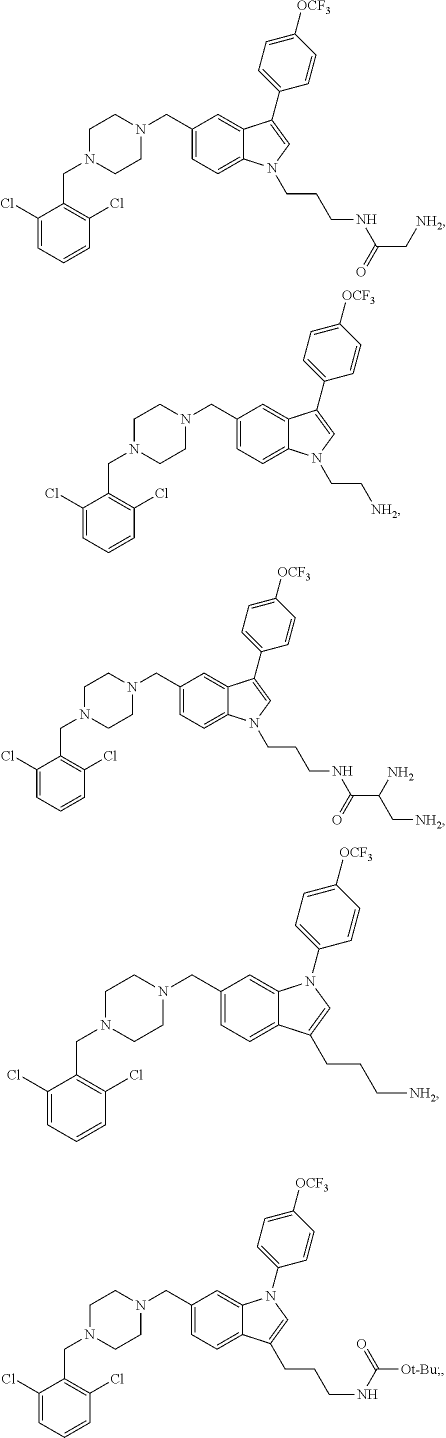

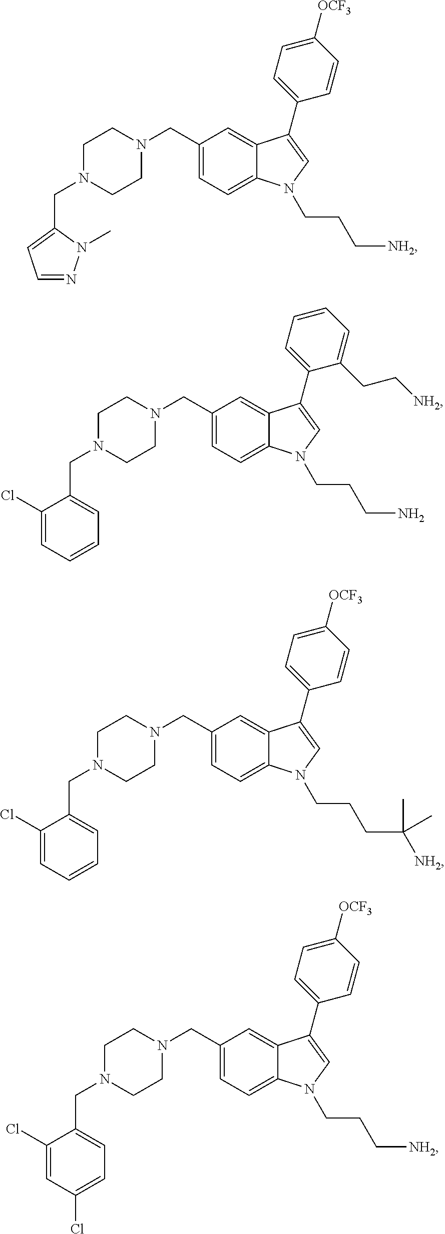 [SMILES: Cn1nccc1CN1CCN(Cc2ccc3c(c2)c(-c2ccc(OC(F)(F)F)cc2)cn3CCCN)CC1 ; NCCCn1cc(-c2ccccc2CCN)c2cc(CN3CCN(Cc4ccccc4Cl)CC3)ccc21 ; CC(C)(N)CCCn1cc(-c2ccc(OC(F)(F)F)cc2)c2cc(CN3CCN(Cc4ccccc4Cl)CC3)ccc21 ; NCCCn1cc(-c2ccc(OC(F)(F)F)cc2)c2cc(CN3CCN(Cc4ccc(Cl)cc4Cl)CC3)ccc21]

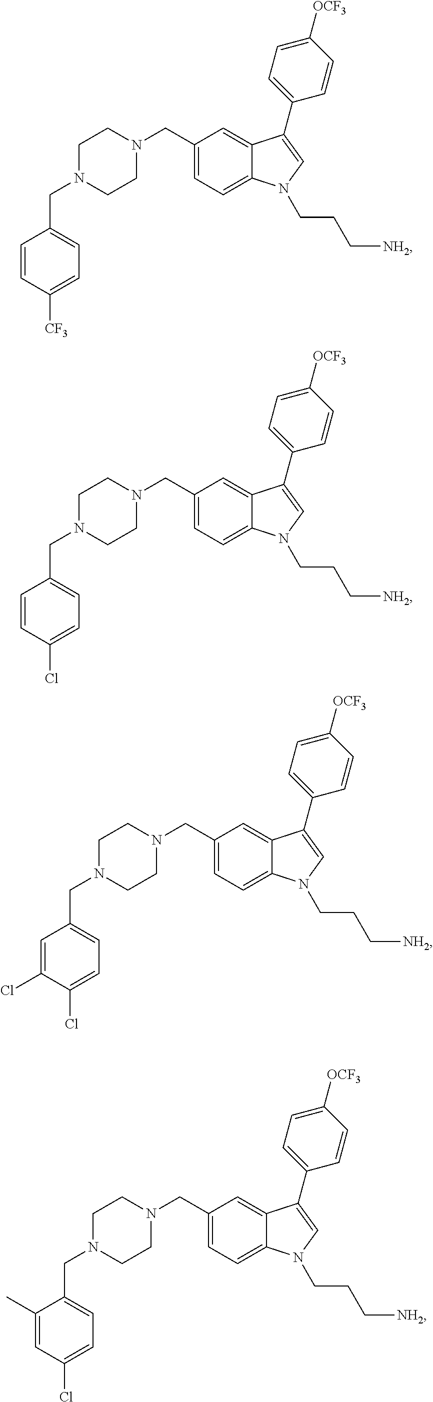 [SMILES: NCCCn1cc(-c2ccc(OC(F)(F)F)cc2)c2cc(CN3CCN(Cc4ccc(C(F)(F)F)cc4)CC3)ccc21 ; NCCCn1cc(-c2ccc(OC(F)(F)F)cc2)c2cc(CN3CCN(Cc4ccc(Cl)cc4)CC3)ccc21 ; NCCCn1cc(-c2ccc(OC(F)(F)F)cc2)c2cc(CN3CCN(Cc4ccc(Cl)c(Cl)c4)CC3)ccc21 ; Cc1cc(Cl)ccc1CN1CCN(Cc2ccc3c(c2)c(-c2ccc(OC(F)(F)F)cc2)cn3CCCN)CC1]

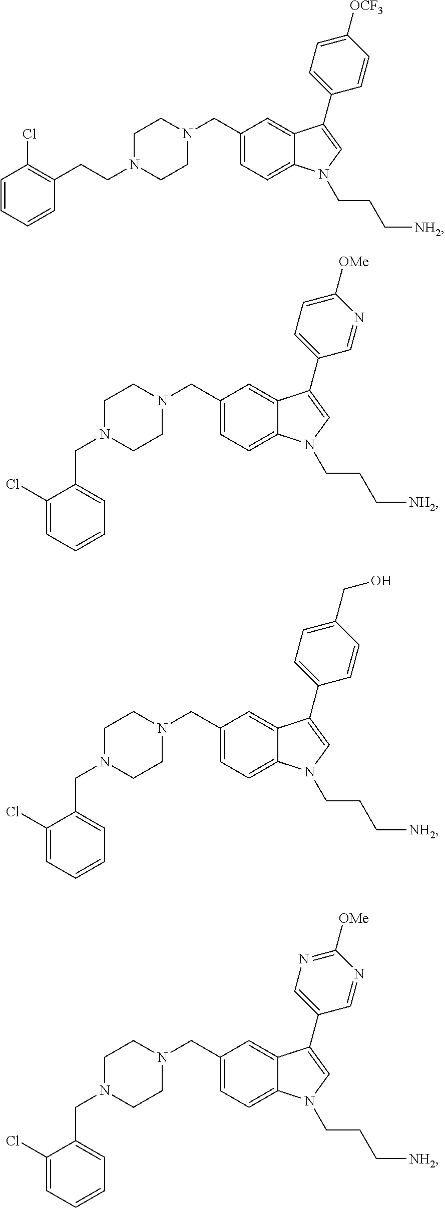 [SMILES: NCCCn1cc(-c2ccc(OC(F)(F)F)cc2)c2cc(CN3CCN(CCc4ccccc4Cl)CC3)ccc21 ; COc1ccc(-c2cn(CCCN)c3ccc(CN4CCN(Cc5ccccc5Cl)CC4)cc23)cn1 ; NCCCn1cc(-c2ccc(CO)cc2)c2cc(CN3CCN(Cc4ccccc4Cl)CC3)ccc21 ; COc1ncc(-c2cn(CCCN)c3ccc(CN4CCN(Cc5ccccc5Cl)CC4)cc23)cn1]

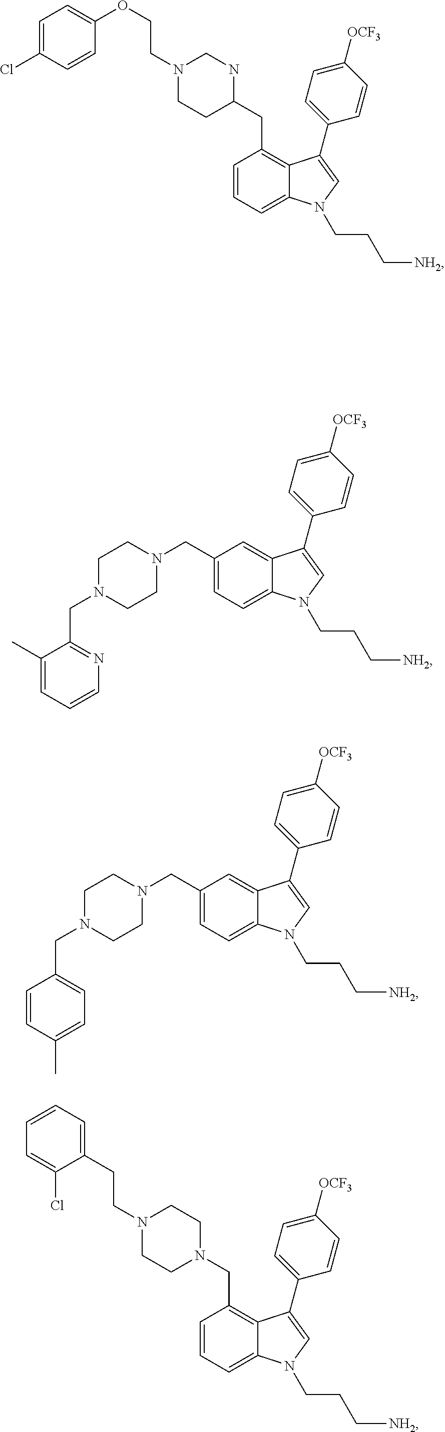

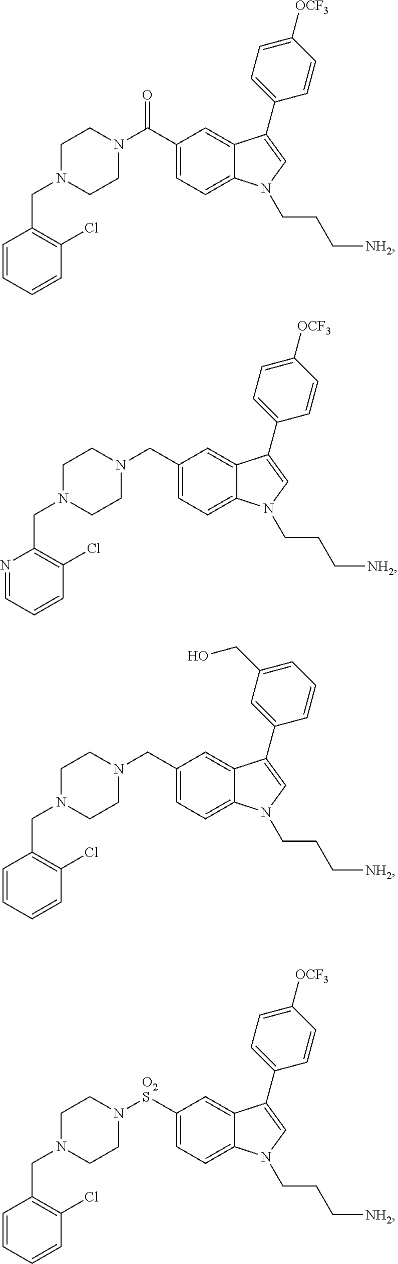 [SMILES: NCCCn1cc(-c2ccc(OC(F)(F)F)cc2)c2cc(C(=O)N3CCN(Cc4ccccc4Cl)CC3)ccc21 ; NCCCn1cc(-c2ccc(OC(F)(F)F)cc2)c2cc(CN3CCN(Cc4ncccc4Cl)CC3)ccc21 ; NCCCn1cc(-c2cccc(CO)c2)c2cc(CN3CCN(Cc4ccccc4Cl)CC3)ccc21 ; NCCCn1cc(-c2ccc(OC(F)(F)F)cc2)c2cc(S(=O)(=O)N3CCN(Cc4ccccc4Cl)CC3)ccc21]

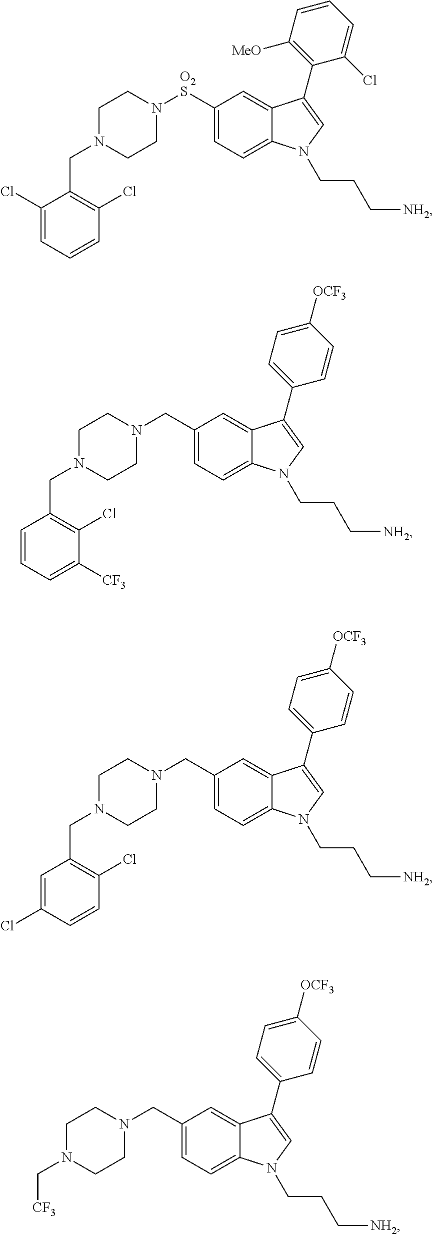 [SMILES: COc1cccc(Cl)c1-c1cn(CCCN)c2ccc(S(=O)(=O)N3CCN(Cc4c(Cl)cccc4Cl)CC3)cc12 ; NCCCn1cc(-c2ccc(OC(F)(F)F)cc2)c2cc(CN3CCN(Cc4cccc(C(F)(F)F)c4Cl)CC3)ccc21 ; NCCCn1cc(-c2ccc(OC(F)(F)F)cc2)c2cc(CN3CCN(Cc4cc(Cl)ccc4Cl)CC3)ccc21 ; NCCCn1cc(-c2ccc(OC(F)(F)F)cc2)c2cc(CN3CCN(CC(F)(F)F)CC3)ccc21]

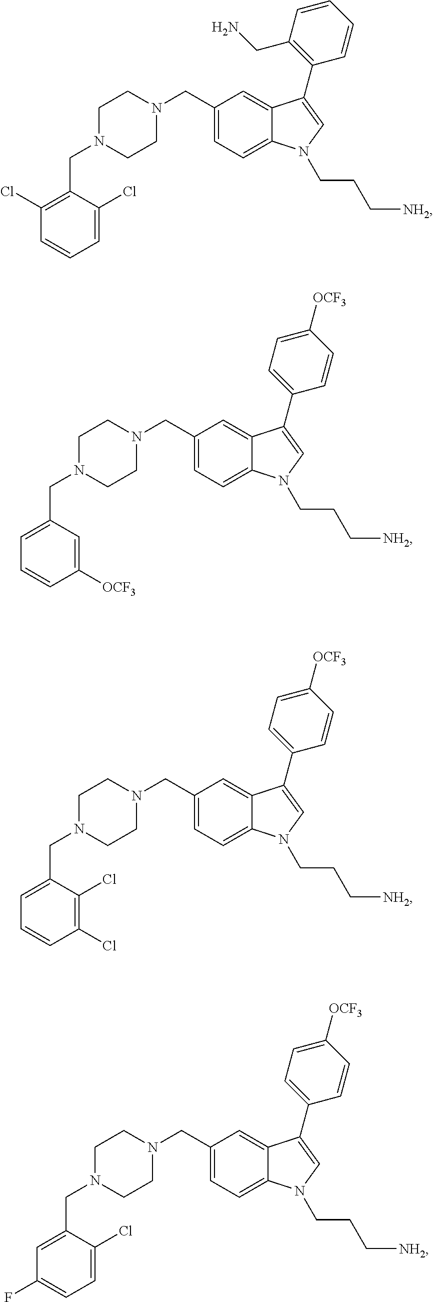 [SMILES: NCCCn1cc(-c2ccccc2CN)c2cc(CN3CCN(Cc4c(Cl)cccc4Cl)CC3)ccc21 ; NCCCn1cc(-c2ccc(OC(F)(F)F)cc2)c2cc(CN3CCN(Cc4cccc(OC(F)(F)F)c4)CC3)ccc21 ; NCCCn1cc(-c2ccc(OC(F)(F)F)cc2)c2cc(CN3CCN(Cc4cccc(Cl)c4Cl)CC3)ccc21 ; NCCCn1cc(-c2ccc(OC(F)(F)F)cc2)c2cc(CN3CCN(Cc4cc(F)ccc4Cl)CC3)ccc21]

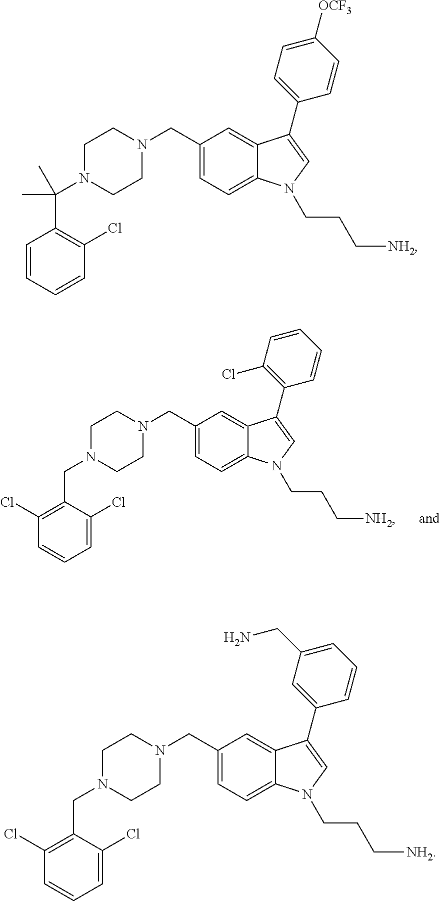 [SMILES: CC(C)(c1ccccc1Cl)N1CCN(Cc2ccc3c(c2)c(-c2ccc(OC(F)(F)F)cc2)cn3CCCN)CC1 ; NCCCn1cc(-c2ccccc2Cl)c2cc(CN3CCN(Cc4c(Cl)cccc4Cl)CC3)ccc21 ; "and" ; NCCCn1cc(-c2cccc(CN)c2)c2cc(CN3CCN(Cc4c(Cl)cccc4Cl)CC3)ccc21]

In some embodiments, the compound of Formula (VIIa) is selected from:

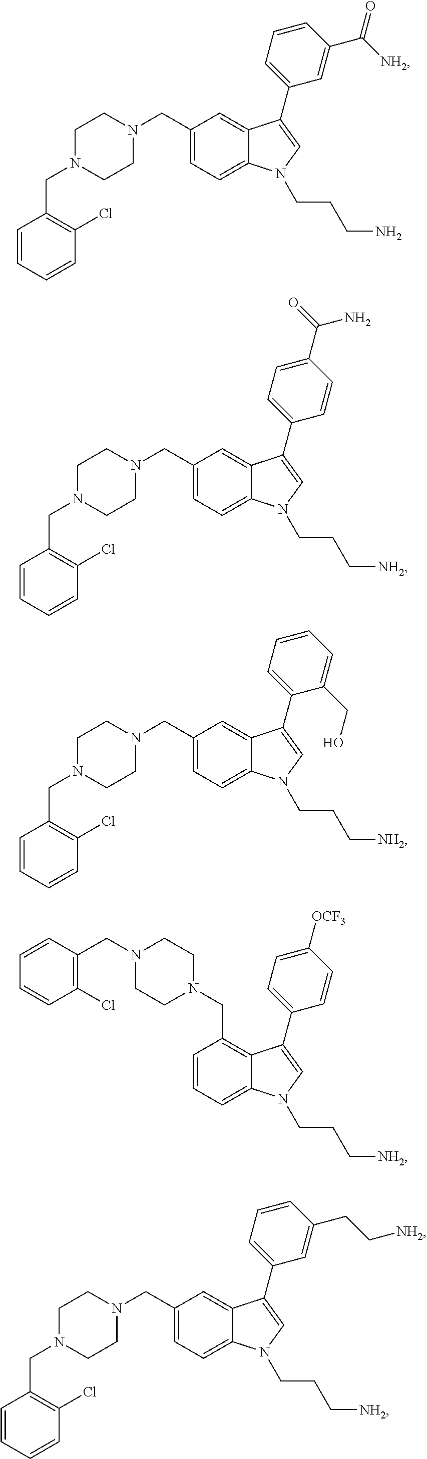 [SMILES: NCCCn1cc(-c2cccc(C(N)=O)c2)c2cc(CN3CCN(Cc4ccccc4Cl)CC3)ccc21 ; NCCCn1cc(-c2ccc(C(N)=O)cc2)c2cc(CN3CCN(Cc4ccccc4Cl)CC3)ccc21 ; NCCCn1cc(-c2ccccc2CO)c2cc(CN3CCN(Cc4ccccc4Cl)CC3)ccc21 ; NCCCn1cc(-c2ccc(OC(F)(F)F)cc2)c2c(CN3CCN(Cc4ccccc4Cl)CC3)cccc21 ; NCCCn1cc(-c2cccc(CCN)c2)c2cc(CN3CCN(Cc4ccccc4Cl)CC3)ccc21]

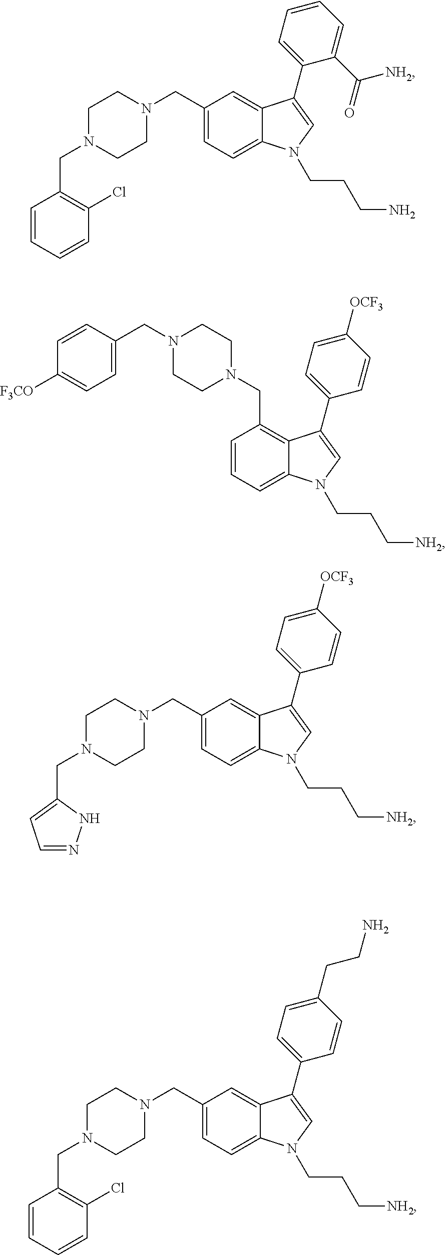 [SMILES: NCCCn1cc(-c2ccccc2C(N)=O)c2cc(CN3CCN(Cc4ccccc4Cl)CC3)ccc21 ; NCCCn1cc(-c2ccc(OC(F)(F)F)cc2)c2c(CN3CCN(Cc4ccc(OC(F)(F)F)cc4)CC3)cccc21 ; NCCCn1cc(-c2ccc(OC(F)(F)F)cc2)c2cc(CN3CCN(Cc4ccn[nH]4)CC3)ccc21 ; NCCCn1cc(-c2ccc(CCN)cc2)c2cc(CN3CCN(Cc4ccccc4Cl)CC3)ccc21]

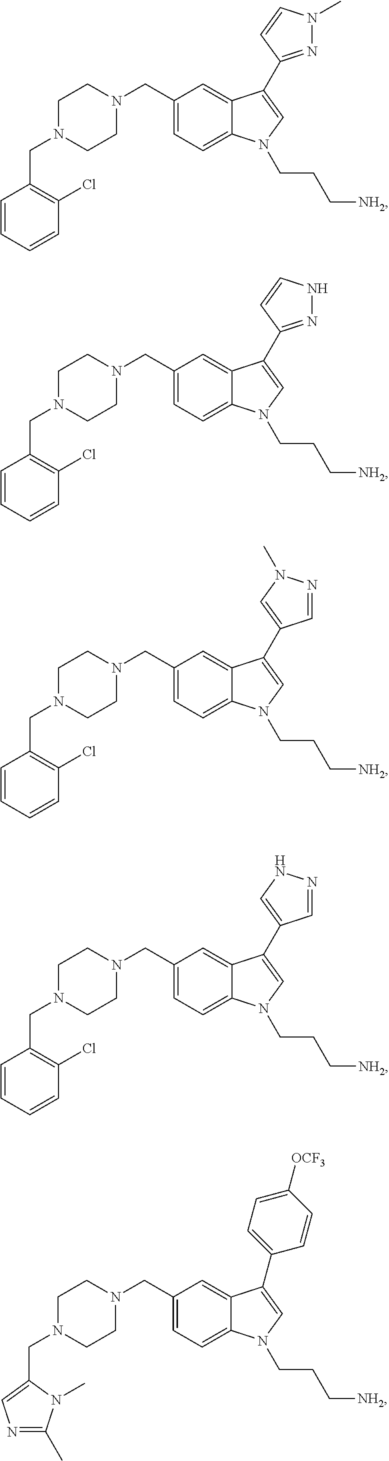 [SMILES: Cn1ccc(-c2cn(CCCN)c3ccc(CN4CCN(Cc5ccccc5Cl)CC4)cc23)n1 ; NCCCn1cc(-c2cc[nH]n2)c2cc(CN3CCN(Cc4ccccc4Cl)CC3)ccc21 ; Cn1cc(-c2cn(CCCN)c3ccc(CN4CCN(Cc5ccccc5Cl)CC4)cc23)cn1 ; NCCCn1cc(-c2cn[nH]c2)c2cc(CN3CCN(Cc4ccccc4Cl)CC3)ccc21 ; Cc1ncc(CN2CCN(Cc3ccc4c(c3)c(-c3ccc(OC(F)(F)F)cc3)cn4CCCN)CC2)n1C]

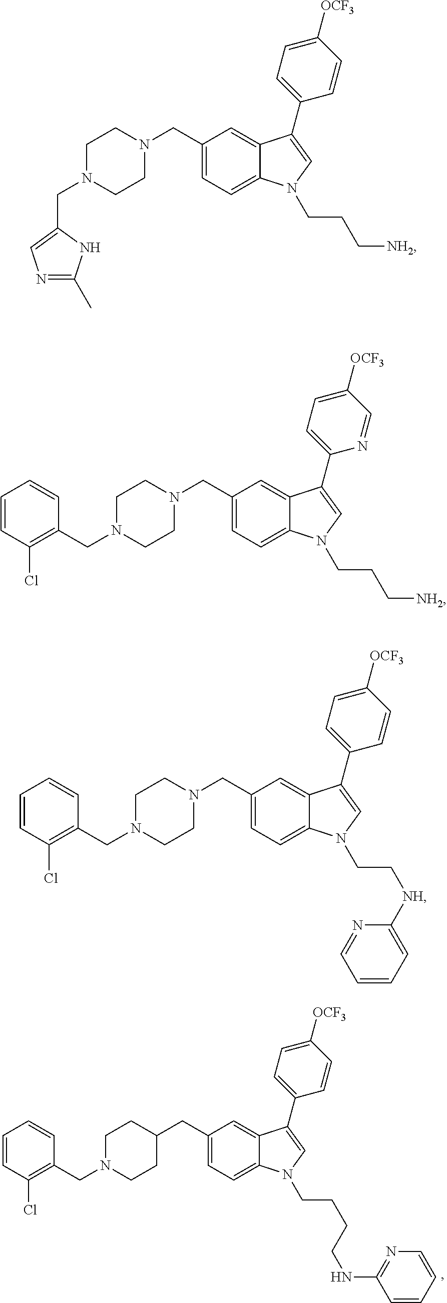 [SMILES: Cc1ncc(CN2CCN(Cc3ccc4c(c3)c(-c3ccc(OC(F)(F)F)cc3)cn4CCCN)CC2)[nH]1 ; NCCCn1cc(-c2ccc(OC(F)(F)F)cn2)c2cc(CN3CCN(Cc4ccccc4Cl)CC3)ccc21 ; FC(F)(F)Oc1ccc(-c2cn(CCNc3ccccn3)c3ccc(CN4CCN(Cc5ccccc5Cl)CC4)cc23)cc1 ; FC(F)(F)Oc1ccc(-c2cn(CCCCNc3ccccn3)c3ccc(CC4CCN(Cc5ccccc5Cl)CC4)cc23)cc1]

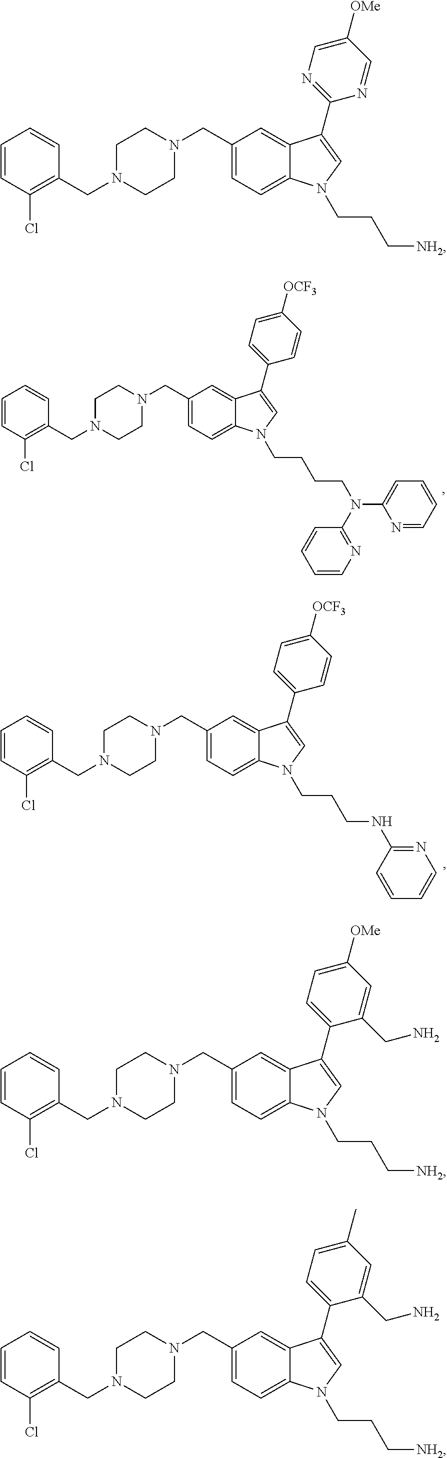

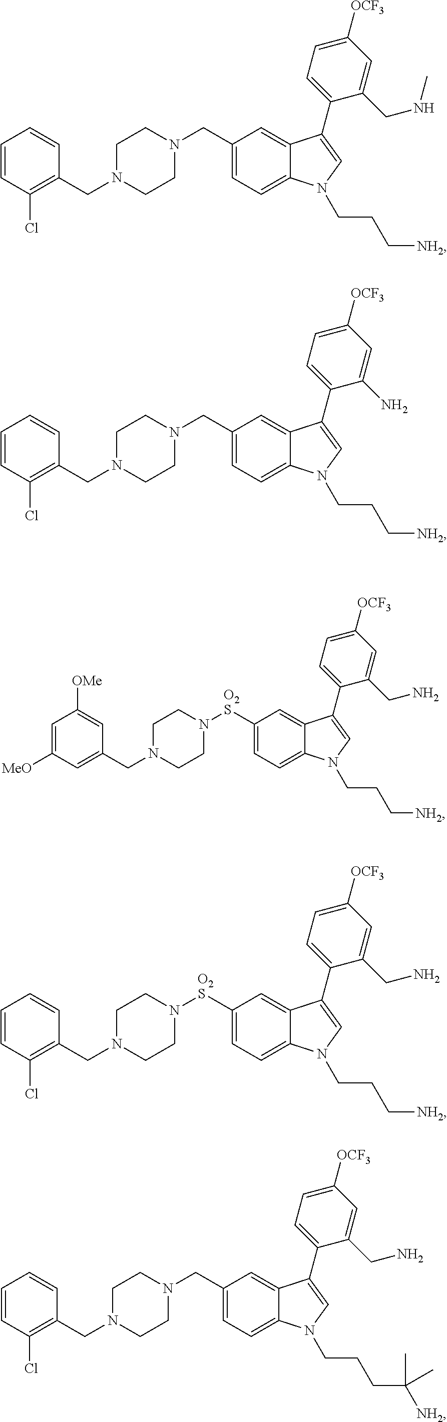 [SMILES: CNCc1cc(OC(F)(F)F)ccc1-c1cn(CCCN)c2ccc(CN3CCN(Cc4ccccc4Cl)CC3)cc12 ; NCCCn1cc(-c2ccc(OC(F)(F)F)cc2N)c2cc(CN3CCN(Cc4ccccc4Cl)CC3)ccc21 ; COc1cc(CN2CCN(S(=O)(=O)c3ccc4c(c3)c(-c3ccc(OC(F)(F)F)cc3CN)cn4CCCN)CC2)cc(OC)c1 ; NCCCn1cc(-c2ccc(OC(F)(F)F)cc2CN)c2cc(S(=O)(=O)N3CCN(Cc4ccccc4Cl)CC3)ccc21 ; CC(C)(N)CCCn1cc(-c2ccc(OC(F)(F)F)cc2CN)c2cc(CN3CCN(Cc4ccccc4Cl)CC3)ccc21]

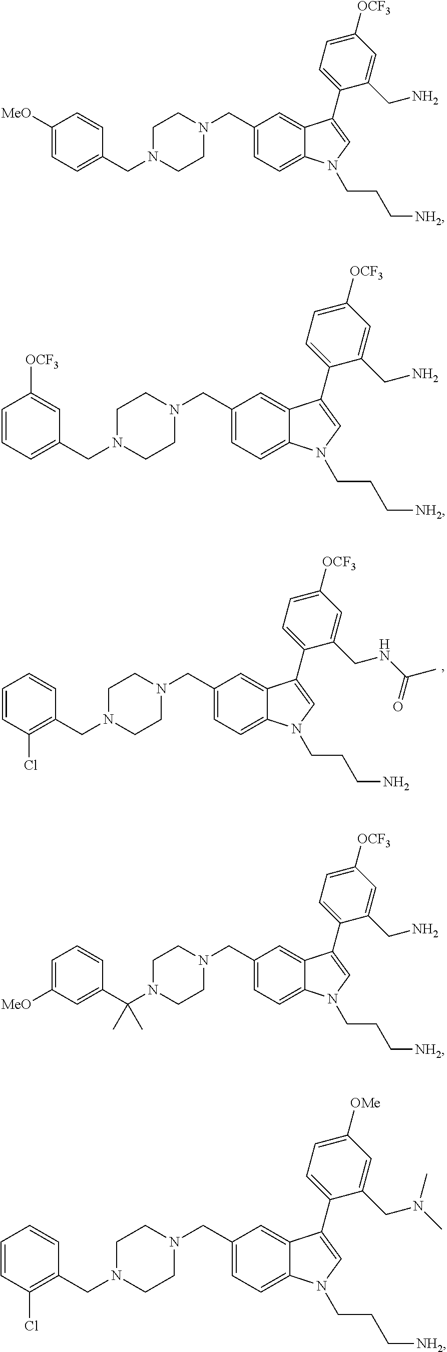 [SMILES: COc1ccc(CN2CCN(Cc3ccc4c(c3)c(-c3ccc(OC(F)(F)F)cc3CN)cn4CCCN)CC2)cc1 ; NCCCn1cc(-c2ccc(OC(F)(F)F)cc2CN)c2cc(CN3CCN(Cc4cccc(OC(F)(F)F)c4)CC3)ccc21 ; CC(=O)NCc1cc(OC(F)(F)F)ccc1-c1cn(CCCN)c2ccc(CN3CCN(Cc4ccccc4Cl)CC3)cc12 ; COc1cccc(C(C)(C)N2CCN(Cc3ccc4c(c3)c(-c3ccc(OC(F)(F)F)cc3CN)cn4CCCN)CC2)c1 ; COc1ccc(-c2cn(CCCN)c3ccc(CN4CCN(Cc5ccccc5Cl)CC4)cc23)c(CN(C)C)c1]

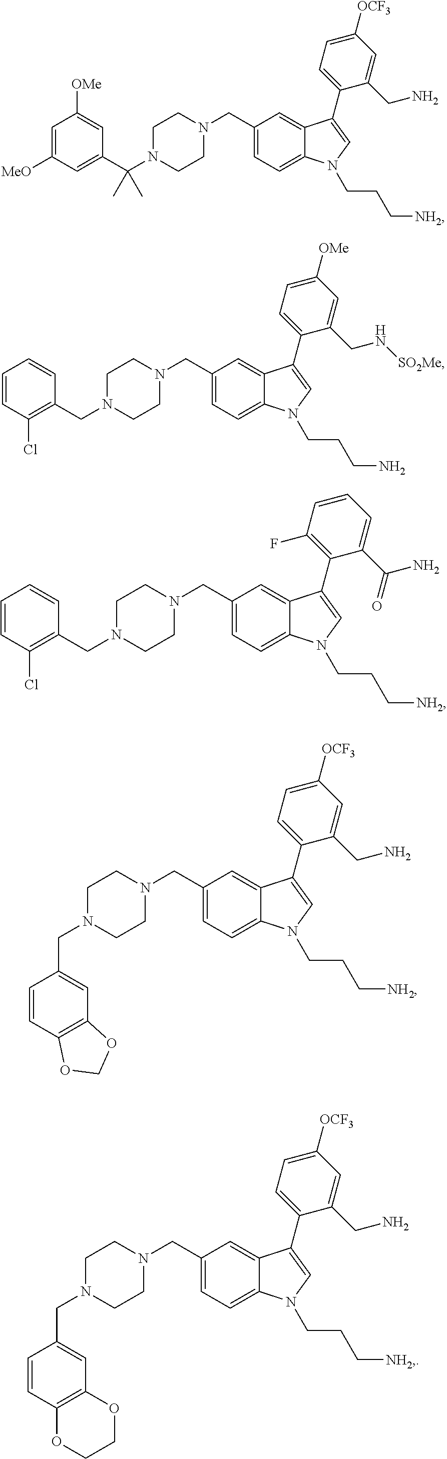

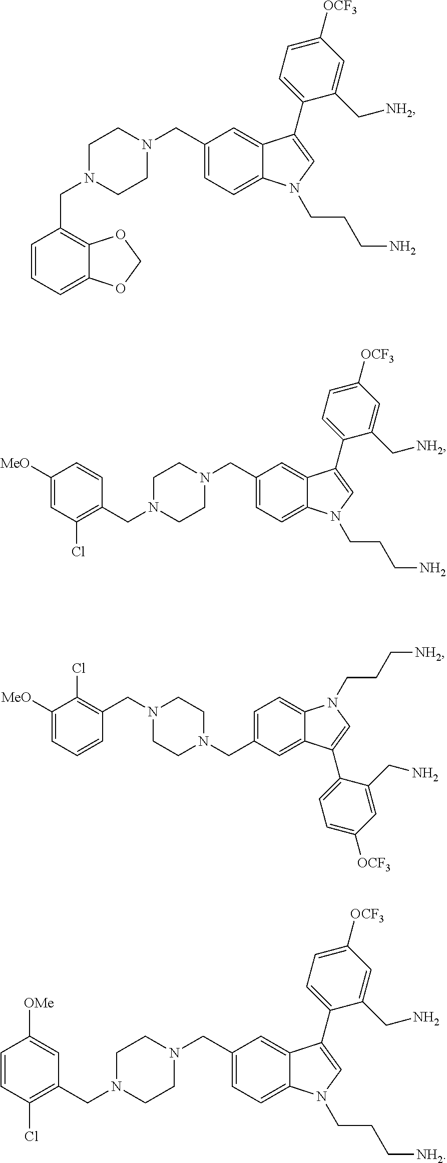 [SMILES: NCCCn1cc(-c2ccc(OC(F)(F)F)cc2CN)c2cc(CN3CCN(Cc4cccc5c4OCO5)CC3)ccc21 ; COc1ccc(CN2CCN(Cc3ccc4c(c3)c(-c3ccc(OC(F)(F)F)cc3CN)cn4CCCN)CC2)c(Cl)c1 ; COc1cccc(CN2CCN(Cc3ccc4c(c3)c(-c3ccc(OC(F)(F)F)cc3CN)cn4CCCN)CC2)c1Cl ; COc1ccc(Cl)c(CN2CCN(Cc3ccc4c(c3)c(-c3ccc(OC(F)(F)F)cc3CN)cn4CCCN)CC2)c1]

In some embodiments, a compound, or pharmaceutically acceptable salt, or solvate thereof disclosed herein selectively binds to a Ras subfamily protein at two or more sites in the G domain of the Ras subfamily protein. In some embodiments, the Ras subfamily protein is HRAS, NRAS, KRAS, RRAS, MRAS, RAP1A, RAP1B, Rap2A, Rap2B, Rap2C, Rit1, Rit2, Rem1, Rem2, Rad, Gem, Rheb1, Rheb2, Noey2, Di-Ras1, Di-Ras2, E-Ras, Rerg, RalA, RalB, NKIRas1, NKIRas2, RasD1 or RasD2. In some embodiments, the Ras subfamily protein is HRAS, KRAS or NRAS. In some embodiments, the compound, or the pharmaceutically acceptable salt, or solvate thereof, selectively binds to the G domain of the Ras subfamily protein. In some embodiments, the compound, or the pharmaceutically acceptable salt, or solvate thereof, selectively binds to a first site on the Ras subfamily protein that comprises at least one amino acid from a switch 1 region. In some embodiments, the first site on the Ras subfamily protein comprises an amino acid residue near a residue similar to D38 of KRAS. In some embodiments, the first site on the Ras subfamily protein comprises an amino acid residue similar to D38 of KRAS. In some embodiments, the first site on the Ras subfamily protein comprises amino acid residue D38 of HRAS, KRAS or NRAS. In some embodiments, the compound, or the pharmaceutically acceptable salt, or solvate thereof, selectively binds to a second site on the RAS subfamily protein that comprises at least one amino acid located between the switch 1 region and a switch 2 region. In some embodiments, the second site on the Ras subfamily protein comprises an amino acid near a residue similar to residue A59 of KRAS. In some embodiments, the second site on the Ras subfamily protein comprises an amino acid residue similar to A59 of the KRAS. In some embodiments, the second site on the Ras subfamily protein comprises amino acid residue A59 of HRAS, KRAS or NRAS. In some embodiments, the compound, or the pharmaceutically acceptable salt, or solvate thereof, selectively binds to an amino acid residue near a residue similar to I21 of KRAS. In some embodiments, the compound, or the pharmaceutically acceptable salt, or solvate thereof, selectively binds to an amino acid residue similar to I21 of KRAS. In some embodiments, the compound, or the pharmaceutically acceptable salt, or solvate thereof, selectively binds to amino acid residue I21 of HRAS, KRAS or NRAS. In some embodiments, the compound, or the pharmaceutically acceptable salt, or solvate thereof, selectively binds to a GTP-bound Ras superfamily protein. In some embodiments, the compound, or the pharmaceutically acceptable salt, or solvate thereof, selectively binds to a non-GDP-bound form of the Ras superfamily protein. In some embodiments, the Ras superfamily protein is an oncogenic mutant. In some embodiments, the Ras superfamily protein is an oncogenic mutant and is HRASG12D, KRASG12D, NRASQ61K, NRASG13V or NRASG13D. In some embodiments, the compound, or the pharmaceutically acceptable salt, or solvate thereof, selectively binds to at least two amino acid residues in the Ras subfamily protein, wherein the at least two amino acid residues are near a residue similar to D38, A59 or I21 of KRAS. In some embodiments, the at least two amino acid residues are similar to D38, A59 or I21 of KRAS. In some embodiments, the at least two amino acid residues are D38, A59 or I21 of HRAS, KRAS or NRAS.

In some embodiments, a compound, or the pharmaceutically acceptable salt, or solvate thereof disclosed herein, selectively binds to a Ras superfamily protein at two or more sites in a Ras superfamily protein comprising a G domain. In some embodiments, the Ras superfamily protein is a protein in the Ras, Rho, Rab, Ran or Arf subfamily. In some embodiments, the Ras superfamily protein is a protein listed in Table 1. In some embodiments, the compound, or the pharmaceutically acceptable salt, or solvate thereof, selectively binds to the G domain of the Ras superfamily protein. In some embodiments, the compound, or the pharmaceutically acceptable salt, or solvate thereof, selectively binds to a first site on the Ras superfamily protein that comprises at least one amino acid in a switch 1 region. In some embodiments, the first site on the Ras superfamily protein comprises an amino acid residue near a residue similar to D38 of KRAS. In some embodiments, the first site on the Ras superfamily protein comprises an amino acid residue similar to D38 of KRAS. In some embodiments, the compound, or the pharmaceutically acceptable salt, or solvate thereof, selectively binds to a second site on the RAS superfamily protein that comprises at least one amino acid located in a region between the switch 1 region and a switch 2 region. In some embodiments, the second site on the Ras superfamily protein comprises an amino acid near a residue similar to A59 of KRAS. In some embodiments, the second site on the Ras superfamily protein comprises an amino acid residue similar to A59 of KRAS. In some embodiments, the compound, or the pharmaceutically acceptable salt, or solvate thereof, selectively binds to an amino acid near a residue similar to I21 of KRAS. In some embodiments, the compound, or the pharmaceutically acceptable salt, or solvate thereof, selectively binds to an amino acid a residue similar to I21 of KRAS. In some embodiments, the compound, or the pharmaceutically acceptable salt, or solvate thereof, selectively binds to a non-GDP-bound form of the Ras superfamily protein. In some embodiments, the compound, or the pharmaceutically acceptable salt, or solvate thereof, is selective for a GTP-bound Ras superfamily protein. In some embodiments, the Ras superfamily protein is an oncogenic mutant. In some embodiments, the compound, or the pharmaceutically acceptable salt, or solvate thereof, selectively binds to at least two amino acid residues in the Ras superfamily protein, wherein the at least two amino acid residues are near a residue similar to D38, A59 or I21 of KRAS. In some embodiments, the at least two amino acid residues are similar to D38, A59 or I21 of KRAS.

Also provided herein is a pharmaceutical composition comprising any one of the compounds disclosed herein or a pharmaceutically acceptable salt, or solvate thereof. In some embodiments, the pharmaceutical composition comprises one or more pharmaceutically acceptable excipients.

Also provided herein is a method for treating or ameliorating the effects of a disease associated with altered Ras signaling, the method comprising administering to a subject in need thereof a pharmaceutical composition described herein, or a pharmaceutically acceptable salt, or solvate thereof. In some embodiments, the disease is cancer, a neurological disorder, a metabolic disorder, an immunological disorder, an inflammatory disorder, or a developmental disorder. In some embodiments, the disease associated with altered Ras signaling is autism, rasopathies, neurofibromatosis type 1, Noonan syndrome, Costello syndrome, cardiofaciocutaneous syndrome, hereditary gingival fibromatosis type 1, Legius syndrome, Leopard syndrome, diabetic retinopathy, diabetes, hyperinsulinemia, chronic idiopathic urticarial, autoimmune lymphoproliferative syndrome, or capillary malformation-arteriovenous malformation. In some embodiments, the cancer is a solid cancer or a hematologic cancer. In some embodiments, the cancer is pancreatic cancer, colorectal cancer, lung cancer, fibrosarcoma, skin cancer, urinary bladder cancer, thyroid cancer, hematopoietic cancer, prostate cancer, breast cancer, liver cancer, soft tissue cancer, leukemia, or bone cancer.

Also provided herein is method for treating or ameliorating a cell proliferative disorder, the method comprising administering a pharmaceutical composition comprising a compound, or a pharmaceutically acceptable salt, or solvate thereof, that selectively binds to at least two amino acid residues of at least two Ras superfamily proteins, wherein each of the Ras superfamily proteins comprises comprising a switch 1 region and a switch 2 region, and wherein the at least two amino acid residues comprise (i) residues near D38 or A59 of KRAS or (ii) residues similar to D38 or A59 of KRAS. In some embodiments, the at least two Ras superfamily proteins are proteins listed in Table 1. In some embodiments, one of the at least two the Ras superfamily proteins is a Ras subfamily protein. In some embodiments, the Ras subfamily protein is HRAS, NRAS, KRAS, RRAS, MRAS, RAP1A, RAP1B, Rap2A, Rap2B, Rap2C, Rit1, Rit2, Rem1, Rem2, Rad, Gem, Rheb1, Rheb2, Noey2, Di-Ras1, Di-Ras2, E-Ras, Rerg, RalA, RalB, NKIRas1, NKIRas2, RasD1 or RasD2. In some embodiments, the Ras subfamily proteins is HRAS, KRAS or NRAS. In some embodiments, the at least two amino acid residues comprise D38 or A59 of KRAS. In some embodiments, the at least two amino acid residues comprise D38, A59 and I21 of KRAS or residues similar to D38, A59 or I21 of KRAS. In some embodiments, the cell proliferative disorder is cancer. In some embodiments, the cancer is a solid cancer or a hematologic cancer. In some embodiments, the cancer is pancreatic cancer, colorectal cancer, lung cancer, fibrosarcoma, skin cancer, urinary bladder cancer, thyroid cancer, hematopoietic cancer, prostate cancer, breast cancer, liver cancer, soft tissue cancer, leukemia, or bone cancer. In some embodiments, the pharmaceutical composition comprises a compound disclosed herein, or a pharmaceutically acceptable salt, or solvate thereof.

Also provided herein is a method for reducing or depleting a population of cancer cells, the method comprising administering a pharmaceutical composition to a subject in need thereof, wherein the pharmaceutical composition comprises a compound disclosed herein, or a pharmaceutically acceptable salt, or solvate thereof. In some embodiments, the cancer cells are from a solid cancer or a hematologic cancer. In some embodiments, the cancer cells are from a pancreatic cancer, colorectal cancer, lung cancer, fibrosarcoma, skin cancer, urinary bladder cancer, thyroid cancer, hematopoietic cancer, prostate cancer, breast cancer, liver cancer, soft tissue cancer, leukemia, or bone cancer.

Also provided herein is use of a compound described herein, or a pharmaceutically acceptable salt, or solvate thereof for the manufacture of a medicament for the treatment of cancer. Also provided herein is use of a compound described herein, or a pharmaceutically acceptable salt, or solvate thereof for treating cancer. Also provided herein is a compound described herein, or a pharmaceutically acceptable salt, or solvate thereof for treating cancer.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: Illustrates a sequence alignment for Ras superfamily proteins (SEQ ID NOS 1-308, respectively, in order of appearance).

### DETAILED DESCRIPTION OF THE INVENTION

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### DEFINITIONS

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms, 6 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers refers to a divalent alkyl radical. Any of the above mentioned monovalent alkyl groups may be an alkylene by abstraction of a second hydrogen atom from the alkyl. In some embodiments, an alkelene is a C₁-C₆alkylene. In other embodiments, an alkylene is a C₁-C₄alkylene. In certain embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises two carbon atoms (e.g., C₂ alkylene). In other embodiments, an alkylene comprises two to four carbon atoms (e.g., C₂-C₄ alkylene). Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized t-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. In some embodiments, cycloalkyl groups include groups having from 3 to 10 ring atoms. In some embodiments, cycloalkyl groups include groups having from 3 to 6 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Polycyclic cycloalkyls include, for example, adamantyl, norbornyl (i.e., bicyclo[2.2.1]heptanyl), norbornenyl, decalinyl, 7,7-dimethyl-bicyclo[2.2.1]heptanyl, and the like.

The term “cycloalkylene” refers to a monocyclic or polycyclic aliphatic, non-aromatic divalent radical, wherein each of the atoms forming the ring (i.e. skeletal atoms) is a carbon atom. In some embodiments, cycloalkylene are spirocyclic or bridged compounds. In some embodiments, cycloalkylenes are optionally fused with an aromatic ring, and the point of attachment is at a carbon that is not an aromatic ring carbon atom. In some embodiments, cycloalkylene groups include groups having from 3 to 10 ring atoms. In some embodiments, cycloalkylene groups include groups having from 3 to 6 ring atoms.

The term “halo” or, alternatively, “halogen” or “halide” means fluoro, chloro, bromo or iodo. In some embodiments, halo is fluoro, chloro, or bromo.

The term “haloalkyl” refers to an alkyl in which one or more hydrogen atoms are replaced by a halogen atom. In one aspect, a fluoralkyl is a C₁-C₆fluoroalkyl.

The term “fluoroalkyl” refers to an alkyl in which one or more hydrogen atoms are replaced by a fluorine atom. In one aspect, a fluoralkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heteroalkylene” refers to an alkylene group in which one or more skeletal atoms of the alkylene are selected from an atom other than carbon, e.g., oxygen, nitrogen (e.g. —NH—, —N(alkyl)-, sulfur, or combinations thereof. In some embodiments, a heteroalkylene is attached to the rest of the molecule at a carbon atom of the heteroalkylene. In one aspect, a heteroalkylene is a C₁-C₆heteroalkylene.

As used herein, the term “heteroatom” refers to an atom of any element other than carbon or hydrogen. In some embodiments, the heteroatom is nitrogen, oxygen, or sulfur. In some embodiments, the heteroatom is nitrogen or oxygen. In some embodiments, the heteroatom is nitrogen.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1 (2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclcic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is a spirocyclic or bridged compound. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from D, halogen, —CN, —NH₂, —NH(alkyl), —CH₂N(alkyl)₂, —N(alkyl)₂, —OH, —CO₂H, —CO₂alkyl, —CH₂NH₂, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —CH₂NH₂, —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CH₂NH₂, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

A “tautomer” refers to a molecule wherein a proton shift from one atom of a molecule to another atom of the same molecule is possible. The compounds presented herein may, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

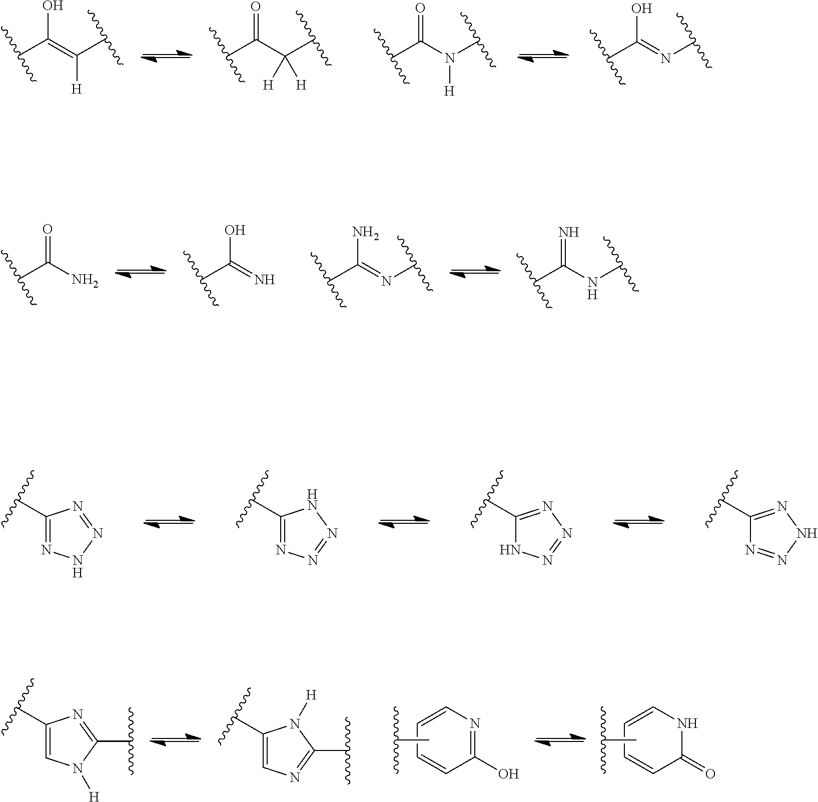

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts. A pharmaceutically acceptable salt of any one of the pyrazole compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

As used herein, “selectively binds”, and grammatical variations thereof, means a binding reaction between two molecules that is at least two times the background and more typically more than 10 to 100 times background molecular associations under physiological conditions. In some embodiments, a compound disclosed herein is “selective” for a given form of a RAS protein and exhibits molecular associations under physiological conditions at least two times the background and more typically more than 10 to 100 times background.

As used herein, “at least one amino acid” from any of the regions or locations of a RAS protein disclosed herein include 1, 2, 3, 4, 5, 6, 7, 8, 9, 10, or more amino acids, up to, and including, the number of amino acids comprising the entire designated region or location of RAS.

As used herein, “near”, as it relates to distances from certain residues, such as D38, A59, or I21, means within about 9 angstroms of the residue, including, but not limited to, within 1, 2, 3, 4, 5, 6, 7, or 8 angstroms of the residue on the RAS protein that corresponds to the amino acid number (such as 38, 59, or 21) of the human HRAS protein.

As used herein, an “oncogenic mutant” is a RAS variant that contains an alteration in the amino acid sequence and has the potential to cause a cell to become cancerous.

As used herein, the phrase “altered RAS signaling” means any deviation in the activity of a RAS protein from that typically observed from wild-type RAS protein in a given tissue. Altered RAS signaling may include, for example, increased RAS signaling or decreased RAS signaling. Altered RAS signaling may be caused by one or more mutations in the RAS protein, such as the oncogenic mutations disclosed above. For example, certain RAS protein mutations may enable RAS protein to constitutively exist in its GTP-bound conformation, either by discouraging interaction of RAS protein with various GAP proteins or by disabling the GTPase activity of RAS protein.

### Ras Family Proteins

In some embodiments, a compound disclosed herein selectively binds to a Ras superfamily protein. Exemplary Ras superfamily proteins are listed in Table 1. In some embodiments, a compound disclosed herein binds to a multiple Ras superfamily proteins listed in Table 1. In some embodiments, a compound disclosed herein binds to multiple Ras subfamily proteins listed in Table 1. In some embodiments, a compound disclosed herein selectively binds to a Ras superfamily protein listed in Table 1 that comprises a G domain. In some embodiments, a compound disclosed herein selectively binds to a Ras superfamily protein comprising a G domain. In some embodiments, a compound disclosed herein selectively binds to a G domain region of a Ras superfamily protein. In some embodiments, a compound disclosed herein selectively binds to the Ras superfamily protein at two or more sites. In some embodiments, a compound disclosed herein selectively binds to the Ras superfamily protein at two or more sites located in the G domain region. In some embodiments, a compound disclosed herein selectively binds to the Ras superfamily protein at two or more sites located in the G domain region. In some embodiments, a compound disclosed herein selectively binds to the Ras superfamily protein at two sites located in the G domain region. In some embodiments, a compound disclosed herein selectively binds to the Ras superfamily protein at three sites located in the G domain region. In some embodiments, a compound disclosed herein selectively binds to the Ras superfamily protein at a first site located in a switch 1 region and a second site located between the switch 1 region and a switch two region of a G domain. In some embodiments, the first site on which a compound disclosed herein binds comprises an amino acid residue near a residue similar to D38 of KRAS. In some embodiments, the first site on which a compound disclosed herein binds comprises an amino acid residue similar to D38 of KRAS. In some embodiments, the first site on which a compound disclosed herein binds comprises amino acid residue D38 of HRAS, KRAS or NRAS. In some embodiments, the second site on which a compound disclosed herein binds comprises an amino acid residue near a residue similar to residue A59 of KRAS. In some embodiments, the second site on which a compound disclosed herein binds comprises an amino acid residue similar to A59 of KRAS. In some embodiments, the second site on which a compound disclosed herein binds comprises amino acid residue A59 of HRAS, KRAS or NRAS. In some embodiments, a third site on which a compound disclosed herein binds comprises an amino acid residue near a residue similar to residue I21 of KRAS. In some embodiments, the third site on which a compound disclosed herein binds comprises an amino acid residue similar to I21 of KRAS. In some embodiments, the third site on which a compound disclosed herein binds comprises amino acid residue I21 of HRAS, KRAS or NRAS. In some embodiments, a third site on which a compound disclosed herein binds comprises an amino acid residue near the Y32 pocket of KRAS. In some embodiments, the third site on which a compound disclosed herein binds comprises an amino acid residue similar to one in the Y32 pocket of KRAS. In some embodiments, the third site on which a compound disclosed herein binds comprises amino acid residue located in the Y32 pocket of HRAS, KRAS or NRAS. In some embodiments, a Ras superfamily protein selectively binds to at least two amino acid residues in the Ras subfamily protein, wherein the at least two amino acid residues are near a residue similar to D38, A59 or I21 of KRAS. In some embodiments, a Ras superfamily protein selectively binds to at least two amino acid residues in the Ras subfamily protein, wherein the at least two amino acid residues are similar to D38, A59 or I21 of KRAS. In some embodiments, a Ras superfamily protein selectively binds to at least two amino acid residues in the Ras subfamily protein, wherein the at least two amino acid are D38, A59 or I21 of KRAS. In some embodiments, a compound disclosed herein selectively binds to a Ras superfamily protein when in a GTP-bound conformation. In some embodiments, a compound disclosed herein selectively binds to a Ras superfamily protein when in a non-GTP-bound conformation. In some embodiments, a compound disclosed herein selectively binds to an oncogenic Ras superfamily protein. Exemplary RAS mutants include HRASG12D, KRASG12D, NRASQ61K, NRASG13V or NRASG13D. In some embodiments, a compound disclosed herein selectively binds to a Ras superfamily protein wherein the Ras superfamily proteins is in the Ras, Rho, Rab, Ran or Arf subfamily. In some embodiments, the compound is a pharmaceutically acceptable salt, or solvate thereof.

TABLE 1

| | | Accession |
|---|---|---|
| Protein | Synonym | Number |
| Ras Subfamily | | NP_005334 |
| H-Ras, isoform 1 | | NP_789765 |
| H-Ras, isoform 2 | H-RasIDX | NP_002515 |
| N-Ras | | NP_004976 |
| K-Ras2B | | NP_203524 |
| K-Ras2A | | NP_006261 |
| R-Ras | | NP_036382 |
| TC21 | R-Ras2 | NP_036351 |
| M-Ras | R-Ras3 | NP_002875 |
| Rap1A | Krev-1/Smgp21 | NP_056461 |
| Rap1B | | NP_066361 |
| Rap2A | | NP_002877 |
| Rap2B | | NP_067006 |
| Rap2C | | NP_008843 |
| Rit1 | Roc1/RibB | NP_002921 |
| Rit2 | Rin/Roc2/RibA | NP_054731 |
| Rem1 | Ges | AAH35663 |
| Rem2 | | NP_004156 |
| Rad | R-Rad/Rem3 | NP_859053 |
| Gem | Kir | NP_005605 |
| Rheb1 | Rheb2 | NP_653194 |
| Rheb2 | RhebL1 | NP_004666 |
| Noey2 | ARHI/RhoI | NP_660156 |
| Di-Ras1 | Rig/GBTS1 | NP_060064 |
| Di-Ras2 | | NP_853510 |
| E-Ras | H-Ras2/H-RasP | NP_116307 |
| Rerg | | NP_005393 |
| RalA, isoform 1 | | AAA36542 |
| RalA, isoform 2 | | NP_002872 |
| RalB | | NP_065078 |
| NKIRas1 | κB-Ras1 | NP_060065 |
| NKIRas2 | κB-Ras2 | NP_057168 |
| RasD1 | DexRas/Ags1 | NP_055125 |
| RasD2 | Rhes/Tem2 | NP_006468 |
| RRP22 | RasL10A | NP_201572 |
| RasL10B | | NP_996563 |
| RasL11A | | NP_076429 |
| RasL11B | | NP_057647 |
| Ris/RasL12 | | NP_079006 |
| Rho Subfamily | | |
| RhoA | ARHA/Rho H12 | NP_001655 |
| RhoB | ARHB/Rho H6 | NP_004031 |
| RhoC | ARHC/Rho H9 | NP_786886 |
| RhoD | ARHD/RhoHP1 | NP_055393 |
| Rnd3 | RhoE/ARHE/Rho8 | NP_005159 |
| Rnd1 | ARHS/Rho6 | NP_055285 |
| Rnd2 | ARHN/RhoN/Rho7 | NP_005431 |
| Rif | ARHF/RhoF | NP_061907 |
| RhoG | ARHG | NP_001656 |
| RhoH | TTF/ARHH | NP_004301 |
| Rac1 | TC25 | NP_008839 |
| Rac1, isoform b | | NP_061485 |
| Rac2 | | NP_002863 |
| Rac3 | | NP_005043 |
| Cdc42, placental | G25K/Cdc42Hs | NP_001782 |
| Cdc42, brain | | NP_426359 |
| TC10 | RhoQ/ARHQ/RasL7A | NP_036381 |
| TCL | TC10β/RhoT/RhoJ/ | NP_065714 |
| | ARHJ/RasL7B | |
| Wrch-1 | RhoU/ARHU/Cdc42L1 | NP_067028 |
| Wrch-2 | Chp/RhoV/ARHV | NP_598378 |
| RhoBTB1 | | NP_055651 |
| RhoBTB2 | DBC2 | NP_055993 |
| Arf family | | |
| Arf1 | | NP_001649 |
| Arf3 | | NP_001650 |
| Arf4 | | NP_001651 |
| Arf5 | | NP_001653 |
| Arf6 | | NP_001654 |
| Sar1a | SARA1/Masra2 | NP_064535 |
| Sar1b | SARA2/Sar1a homolog | NP_057187 |
| Arl1 | 2/CMRD | NP_001168 |
| Arl2 | | NP_001658 |
| Arl3 | | NP_004302 |
| Arl4 | | NP_997625 |
| Arl5, isoform 1 | | NP_036229 |
| Arl5, isoform 2 | | NP_817114 |
| Arl6 | BBS3 | NP_816931 |
| Arl7 LAK | | NP_005728 |
| Arl10B | Gie2 | NP_620150 |
| Arl10C | Gie1 | NP_060654 |
| Ard1, isoform α | ArfD1/Trim23/RNF46 | NP_001647 |
| Ard1, isoform β | | NP_150230 |
| Ard1, isoform γ | | NP_150231 |
| Arf4L | | NP_001652 |
| ArfRP1 | Arp | NP_003215 |
| Rab family | | |
| Rab1A | | NP_004152 |
| Rab1B | | NP_112243 |
| Rab2A | | NP_002856 |
| Rab3A | | NP_002857 |
| Rab3B | | NP_002858 |
| Rab3C | | NP_612462 |
| Rab3D | GOV/D2-2/Rab16/Rad3D | NP_004274 |
| Rab4A | | NP_004569 |
| Rab4B | | NP_057238 |
| Rab5A | | NP_004153 |
| Rab5B | | NP_002859 |
| Rab5C, isoform a | RabL/Rab5CL | NP_958842 |
| Rab5C, isoform b | | NP_004574 |
| Rab6A, isoform a | | NP_002860 |
| Rab6A, isoform b | | NP_942599 |
| Rab6B | | NP_057661 |
| Rab6C | WTH3 | NP_115520 |
| Rab7A | | NP_004628 |
| Rab7B | | NP_796377 |
| Rab8A | | NP_005361 |
| Rab8B | MEL | NP_057614 |
| Rab9A | | NP_004242 |
| Rab10 | | NP_057215 |
| Rab11A | YL8 | NP_004654 |
| Rab11B | H-YPT3 | NP_004209 |
| Rab12 | | XP_113967 |
| Rab13 | | NP_002861 |
| Rab14 | FBP | NP_057406 |
| Rab15 | | NP_941959 |
| Rab17 | | NP_071894 |
| Rab18 | | NP_067075 |
| Rab21 | | NP_055814 |
| Rab22A | | NP_065724 |
| Rab23 | HSPC137 | NP_057361 |
| Rab24 | | AAH21263 |
| Rab25 | CATX-8 | AAH33322 |
| Rab26 | | NP_055168 |
| Rab27A | Ram | NP_899059 |
| Rab27B | | NP_004154 |
| Rab28 | | NP_004240 |
| Rab30 | | NP_055303 |
| Rab32 | | NP_006825 |
| Rab33A | RabS10 | NP_004785 |
| Rab33B | | NP_112586 |
| Rab34 | Rah/Rab39 | NP_114140 |
| Rab35 | Ray/H-ray/Rab1C | NP_006852 |
| Rab36 | | NP_004905 |
| Rab37 | | NP_783865 |
| Rab38 | NY-MEL-1 | NP_071732 |
| Rab39A | Rar3/RarL/RasL8C | NP_059986 |
| Rab40C | | NP_066991 |
| Rab7L1 | Rab29(rat) | NP_003920 |
| Ran family | | |
| Ran | | NP_006316 |
| Unclassified | | |
| Miro-1 | RhoT1 | NP_060777 |
| Miro-2 | RhoT2 | NP_620124 |
| Rab20 | | NP_060287 |
| RabL2B, isoform 1 | | NP_001003789 |

In some embodiments, a compound disclosed herein selectively binds to a Ras subfamily protein. Exemplary Ras subfamily proteins are listed in the first portion of Table 1. Exemplary Ras subfamily proteins are listed in the first portion of Table 1. In some embodiments, a compound disclosed herein selectively binds to a Ras subfamily protein listed in Table 1 that comprises a G domain. In some embodiments, a compound disclosed herein selectively binds to a Ras subfamily protein comprising a G domain. In some embodiments, a compound disclosed herein selectively binds to a G domain region of a Ras subfamily protein. In some embodiments, a compound disclosed herein selectively binds to the Ras subfamily protein at two or more sites. In some embodiments, a compound disclosed herein selectively binds to the Ras subfamily protein at two or more sites located in the G domain region. In some embodiments, a compound disclosed herein selectively binds to the Ras subfamily protein at two sites located in the G domain region. In some embodiments, a compound disclosed herein selectively binds to the Ras subfamily protein at three sites located in the G domain region. In some embodiments, a compound disclosed herein selectively binds to the Ras subfamily protein at a first site located in a switch 1 region and a second site located between the switch 1 region and a switch two region of a G domain. In some embodiments, the first site on which a compound disclosed herein binds comprises an amino acid residue near a residue similar to D38 of KRAS. In some embodiments, the first site on which a compound disclosed herein binds comprises an amino acid residue similar to D38 of KRAS. In some embodiments, the first site on which a compound disclosed herein binds comprises amino acid residue D38 of HRAS, KRAS or NRAS. In some embodiments, the second site on which a compound disclosed herein binds comprises an amino acid residue near a residue similar to residue A59 of KRAS. In some embodiments, the second site on which a compound disclosed herein binds comprises an amino acid residue similar to A59 of KRAS. In some embodiments, the second site on which a compound disclosed herein binds comprises amino acid residue A59 of HRAS, KRAS or NRAS. In some embodiments, a third site on which a compound disclosed herein binds comprises an amino acid residue near a residue similar to residue I21 of KRAS. In some embodiments, the third site on which a compound disclosed herein binds comprises an amino acid residue similar to I21 of KRAS. In some embodiments, the third site on which a compound disclosed herein binds comprises amino acid residue I21 of HRAS, KRAS or NRAS. In some embodiments, a third site on which a compound disclosed herein binds comprises an amino acid residue near the Y32 pocket of KRAS. In some embodiments, the third site on which a compound disclosed herein binds comprises an amino acid residue similar to one in the Y32 pocket of KRAS. In some embodiments, the third site on which a compound disclosed herein binds comprises amino acid residue located in the Y32 pocket of HRAS, KRAS or NRAS. In some embodiments, a Ras subfamily protein selectively binds to at least two amino acid residues in the Ras subfamily protein, wherein the at least two amino acid residues are near a residue similar to D38, A59 or I21 of KRAS. In some embodiments, a Ras subfamily protein selectively binds to at least two amino acid residues in the Ras subfamily protein, wherein the at least two amino acid residues are similar to D38, A59 or I21 of KRAS. In some embodiments, a Ras subfamily protein selectively binds to at least two amino acid residues in the Ras subfamily protein, wherein the at least two amino acid are D38, A59 or I21 of KRAS. In some embodiments, a compound disclosed herein selectively binds to a Ras subfamily protein when in a GTP-bound conformation. In some embodiments, a compound disclosed herein selectively binds to a Ras subfamily protein when in a non-GTP-bound conformation. In some embodiments, a compound disclosed herein selectively binds to an oncogenic Ras subfamily protein. Exemplary RAS mutants include HRASG12D, KRASG12D, NRASQ61K, NRASG13V or NRASG13D. In some embodiments, a compound disclosed herein selectively binds to a Ras subfamily protein wherein the Ras subfamily proteins is HRAS, NRAS, KRAS, RRAS, MRAS, RAP1A, RAP1B, Rap2A, Rap2B, Rap2C, Rit1, Rit2, Rem1, Rem2, Rad, Gem, Rheb1, Rheb2, Noey2, Di-Ras1, Di-Ras2, E-Ras, Rerg, RalA, RalB, NKIRas1, NKIRas2, RasD1 or RasD2. In some embodiments, the compound is a pharmaceutically acceptable salt, or solvate thereof.

### Ras Signaling Pathway

In some embodiments, a compound disclosed herein selectively binds to a Ras superfamily protein and alters a downstream signaling pathway. In some embodiments, the selective binding to the Ras superfamily family, alters signaling of RAF, Ral, MEKK, SEK, MEK, ERK, JNK, p38, Cdc25, PLD, AF6, PKC-gamma, NFkB, Nore1, Rin1, PI3K, GAP, Rho, ROCKs, Rac, Cdc42, or PKB/Akt. In some embodiments, a compound disclosed herein selectively binds to a Ras subfamily protein and alters a downstream signaling pathway. In some embodiments, the selective binding to the Ras subfamily family, alters signaling of RAF, Ral, RalA, MEKK, SEK, MEK, ERK, JNK, p38, Cdc25, PLD, AF6, PKC-gamma, NFkB, Nore1, Rin1, PI3K, GAP, Rho, ROCKs, Rac, Cdc42, or PKB/Akt. In some cases, the Ras subfamily protein is HRAS, NRAS, or KRAS.

In some embodiments, a compound disclosed herein selectively binds to a Ras superfamily protein and disrupts binding with an effector protein. In some cases, the effector protein binds to the Ras superfamily protein when in a GTP-bound state. In some embodiments, a compound disclosed herein selectively binds to a Ras subfamily protein and disrupts binding with an effector protein. In some cases, the effector protein binds to the Ras subfamily protein when in a GTP-bound state. In cases, the effector protein is a Raf kinase, phosphatidylinositol 3-kinase (PI3K), RalGEF or NORE/MST1. In some cases, the Ras subfamily protein is HRAS, NRAS, or KRAS.

In some embodiments, a compound disclosed herein selectively binds to a Ras superfamily protein and alters activity of a cellular function. In some embodiments, a compound disclosed herein selectively binds to a Ras subfamily protein and alters activity of a cellular function. In some cases, the Ras subfamily protein is HRAS, NRAS, or KRAS. Exemplary cellular functions altered include cytoskeletal organization, transcription, apoptosis, cell cycle progression, golgi trafficking vesicle formation, and cell-cell junction interactions. Where the increase or lack of a cellular function is correlated with a diseases state, the selective binding of a compound disclosed herein results in inhibiting a deleterious activity associated with the diseases state.

Diseases Associated with Altered RAS Signaling

In some cases, a compound disclosed herein is used to treat or ameliorate a disease associated with altered RAS signaling when administered to a subject in need thereof. In some cases, a compound disclosed herein is used to treat or ameliorate the effects of a disease associated with altered RAS signaling when administered to a subject in need thereof. Exemplary disease associated with altered RAS signaling include cancer, a neurological disorder, a metabolic disorder, an immunological disorder, an inflammatory disorder, and a developmental disorder. Preferably, the disease is selected from the group consisting of autism, rasopathies, neurofibromatosis type 1, Noonan syndrome, Costello syndrome, cardiofaciocutaneous syndrome, hereditary gingival fibromatosis type 1, Legius syndrome, Leopard syndrome, diabetic retinopathy, diabetes, hyperinsulinemia, chronic idiopathic urticarial, autoimmune lymphoproliferative syndrome, and capillary malformation-arteriovenous malformation.

In some cases, a compound disclosed herein is used to treat or ameliorate a cancer when administered to a subject in need thereof. Exemplary cancers include both solid cancers and hemotologic cancers. Non-limiting examples of solid cancers include adrenocortical carcinoma, anal cancer, bladder cancer, bone cancer (such as osteosarcoma), brain cancer, breast cancer, carcinoid cancer, carcinoma, cervical cancer, colon cancer, colorectal cancer, endometrial cancer, esophageal cancer, extrahepatic bile duct cancer, Ewing family of cancers, extracranial germ cell cancer, eye cancer, gallbladder cancer, gastric cancer, germ cell tumor, fibrosarcoma, gestational trophoblastic tumor, head and neck cancer, hypopharyngeal cancer, islet cell carcinoma, kidney cancer, large intestine cancer, laryngeal cancer, leukemia, lip and oral cavity cancer, liver cancer, lung cancer, lymphoma, malignant mesothelioma, Merkel cell carcinoma, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell cancer, pancreatic cancer, paranasal sinus and nasal cavity cancer, parathyroid cancer, penile cancer, pituitary cancer, plasma cell neoplasm, prostate cancer, rhabdomyosarcoma, rectal cancer, renal cell cancer, transitional cell cancer of the renal pelvis and ureter, salivary gland cancer, Sézary syndrome, skin cancers (such as cutaneous t-cell lymphoma, Kaposi's sarcoma, mast cell tumor, and melanoma), small intestine cancer, soft tissue sarcoma, stomach cancer, testicular cancer, thymoma, thyroid cancer, urethral cancer, uterine cancer, vaginal cancer, vulvar cancer, and Wilms' tumor. Examples of hematologic cancers include, but are not limited to, leukemias, such as adult/childhood acute lymphoblastic leukemia, adult/childhood acute myeloid leukemia, chronic lymphocytic leukemia, chronic myelogenous leukemia, and hairy cell leukemia, lymphomas, such as AIDS-related lymphoma, cutaneous T-cell lymphoma, adult/childhood Hodgkin lymphoma, mycosis fungoides, adult/childhood non-Hodgkin lymphoma, primary central nervous system lymphoma, Sézary syndrome, cutaneous T-cell lymphoma, and Waldenstrom macroglobulinemia, as well as other proliferative disorders such as chronic myeloproliferative disorders, Langerhans cell histiocytosis, multiple myeloma/plasma cell neoplasm, myelodysplastic syndromes, and myelodysplastic/myeloproliferative neoplasms. In some cases, a compound disclosed herein is used to treat or ameliorate a cell proliferative disorder when administered to a subject in need thereof. In some cases, the cell proliferative disorder is a cancer.

### Compounds

Compounds described herein, including pharmaceutically acceptable salts, and pharmaceutically acceptable solvates thereof, that modulate Ras signaling.

In one aspect, provided herein is a compound of Formula (Ia), or a pharmaceutically acceptable salt, or solvate thereof:

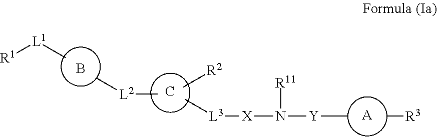

wherein,

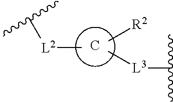

is a bicyclic heteroaryl that is selected from the following structures:

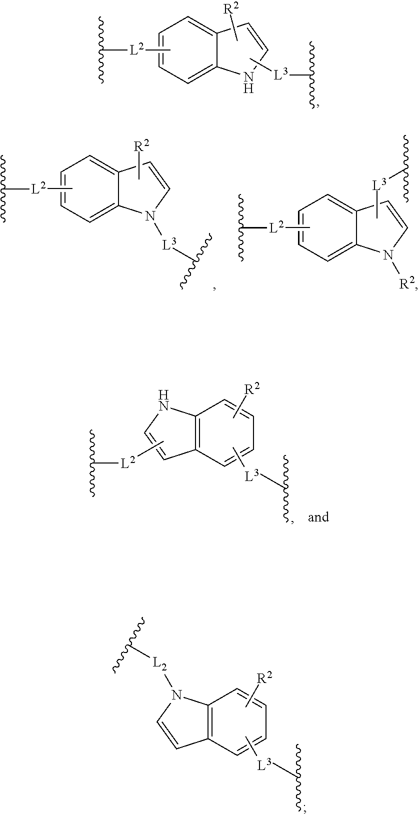

L¹ and L² are each independently an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N with the proviso that Ring B is not:

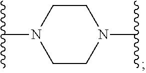 [SMILES: *N1CCN(*)CC1]

wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)— or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N;
wherein if Ring A is substituted, then Ring A is substituted with at least one R^{A};
each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring, and
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring,
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl).

In some embodiments,

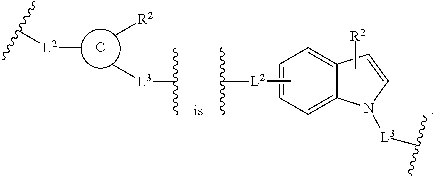

In some embodiments

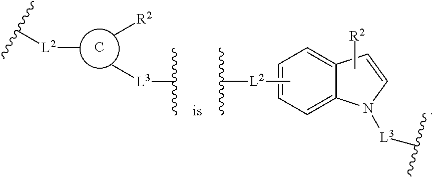

In some embodiments, a compound of Formula (Ia) has the following structure of Formula (Ib), or a pharmaceutically acceptable salt, or solvate thereof:

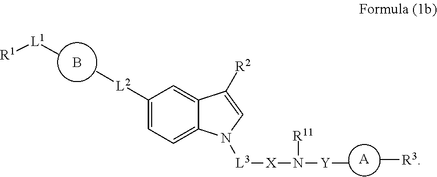

In some embodiments, a compound of Formula (Ia) has the following structure of Formula (Ic), or a pharmaceutically acceptable salt, or solvate thereof:

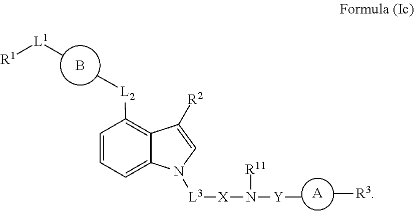

In some embodiments,

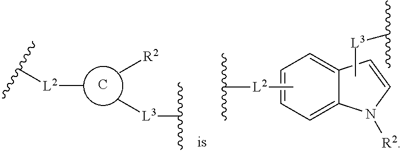

In some embodiments,

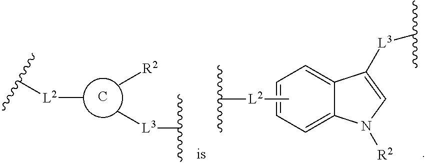

In some embodiments, a compound of Formula (Ia) has the following structure of Formula (Id), or a pharmaceutically acceptable salt, or solvate thereof:

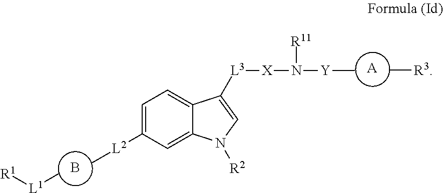

In some embodiments, a compound of Formula (Ia) has the following structure of Formula (Ie), or a pharmaceutically acceptable salt, or solvate thereof:

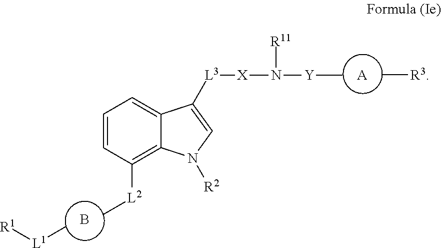

In some embodiments,

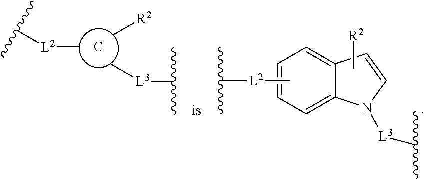

In some embodiments,

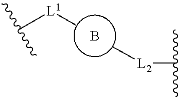

is selected from the following:

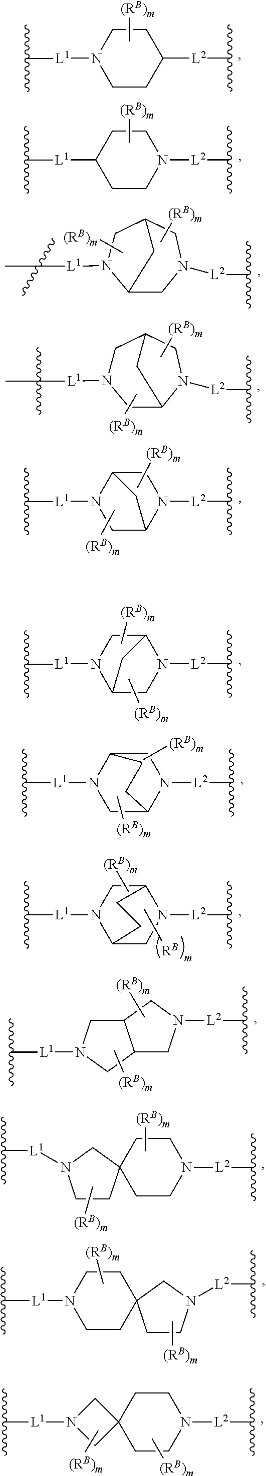

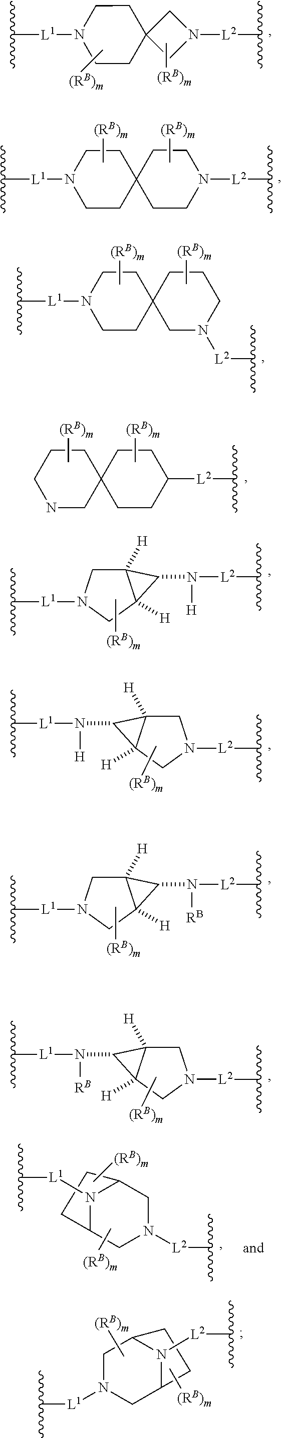

and each m is independently 0, 1, 2, 3, or 4.

In some embodiments,

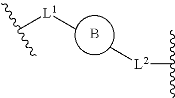

is selected from the following:

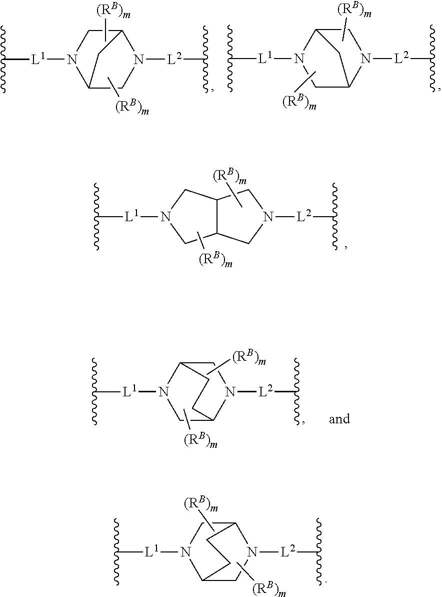

In some embodiments,

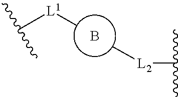

is selected from the following:

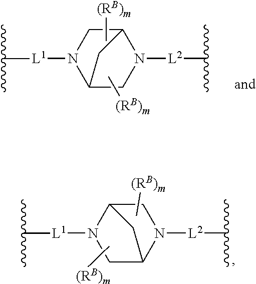

In some embodiments,

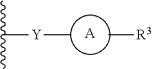

is selected from the following:

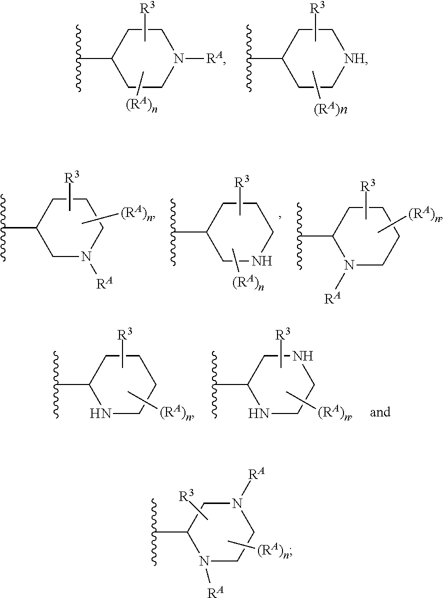

and each n is independently 0, 1, 2, 3, or 4.

In some embodiments,

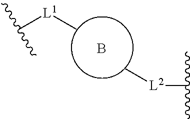

is selected from the following:

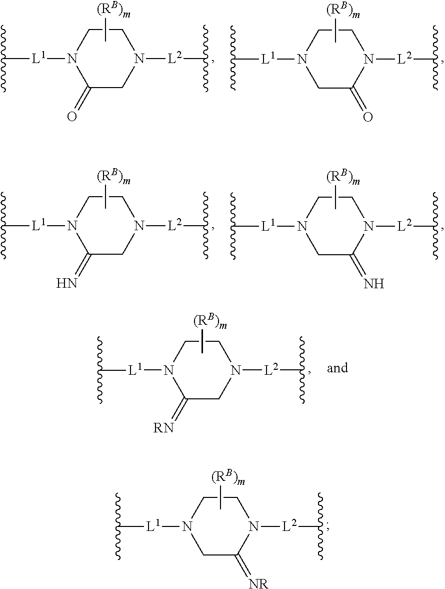

and

R is optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R is C₁-C₄alkyl.

In some embodiments,

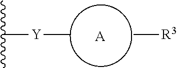

is selected from the following:

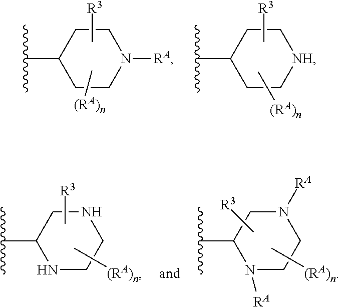

In some embodiments,

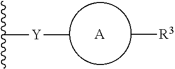

is selected from the following:

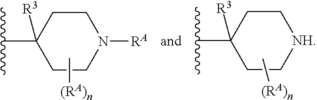

In some embodiments, L¹ is —CH₂—, C(═O), O, S, S(═O), S(═O)₂, or NR⁴. In some embodiments, L¹ is —CH₂—. In some embodiments, L² is —CH₂—, C(═O), O, S, S(═O), S(═O)₂, or NR₄. In some embodiments, L² is —CH₂—. In some embodiments, L³ is absent, —CH₂—, —CH₂—CH₂—, or —CH₂—CH₂—CH₂—. In some embodiments, L³ is —CH₂—CH₂—.

In some embodiments, L³ is:

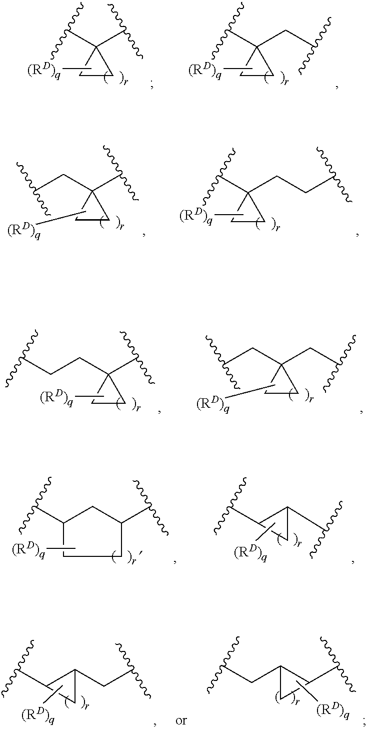

each q is independently 0, 1, 2, 3, or 4;

r is 1, 2, 3, 4, or 5, and

r′ is 1 or 2.

In some embodiments, X is —CH₂— or C(═O). In some embodiments, X is —CH₂—.

In some embodiments, X is:

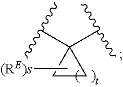

each s is independently 0, 1, 2, 3, or 4; and

t is 1, 2, 3, 4, or 5.

In some embodiments, X is:

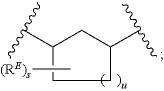

and

each s is independently 0, 1, 2, 3, or 4; and

u is 0, 1, or 2.

In some embodiments, L³-X is —CH₂—CH₂—CH₂—. In some embodiments, Y is —CH₂— or C(═O). In some embodiments, Y is C(═O). In some embodiments, R¹¹ is hydrogen.

In some embodiments, R³ is H. In some embodiments, R³ is CH₂N(R⁹)(R¹⁰). In some embodiments, R³ is N(R⁹)(R¹⁰).

In some embodiments, R⁹ and R¹⁰ are each H. In some embodiments, R⁹ is H and R¹⁰ is —C₁-C₄alkylene-(optionally substituted phenyl) or —C₁-C₄alkylene-(optionally substituted heteroaryl). In some embodiments, R⁹ is H and R¹⁰ is —CH₂-(optionally substituted phenyl). In some embodiments, R⁹ is H and R¹⁰ is —CH₂-(optionally substituted heteroaryl).

In some embodiments, R³ is CH₂N(R⁹)(R¹⁰); and R⁹ and R¹⁰ are each H. In some embodiments, R³ is CH₂N(R⁹)(R¹⁰); and R⁹ is H and R¹⁰ is —C₁-C₄alkylene-(optionally substituted phenyl) or —C₁-C₄alkylene-(optionally substituted heteroaryl). In some embodiments, R³ is CH₂N(R⁹)(R¹⁰); and R⁹ is H and R¹⁰ is —C₁-C₄alkylene-(optionally substituted phenyl). In some embodiments, R³ is CH₂N(R⁹)(R¹⁰); and R⁹ is H and R¹⁰ is —C₁-C₄alkylene-(optionally substituted heteroaryl). In some embodiments, R³ is N(R⁹)(R¹⁰); and R⁹ and R¹⁰ are each H. In some embodiments, R³ is N(R⁹)(R¹⁰); and R⁹ is H and R¹⁰ is —C₁-C₄alkylene-(optionally substituted phenyl) or —C₁-C₄alkylene-(optionally substituted heteroaryl). In some embodiments, R³ is N(R⁹)(R¹⁰); and R⁹ is H and R¹⁰ is —C₁-C₄alkylene-(optionally substituted phenyl). In some embodiments, R³ is N(R⁹)(R¹⁰); and R⁹ is H and R¹⁰ is —C₁-C₄alkylene-(optionally substituted heteroaryl).

In some embodiments, R¹ is an unsubstituted phenyl. In some embodiments, R¹ is a substituted phenyl. In some embodiments, R¹ is selected from:

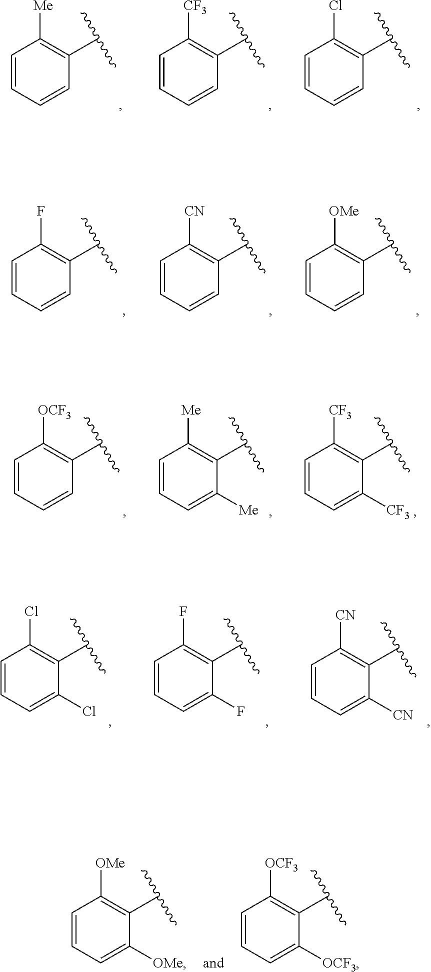

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

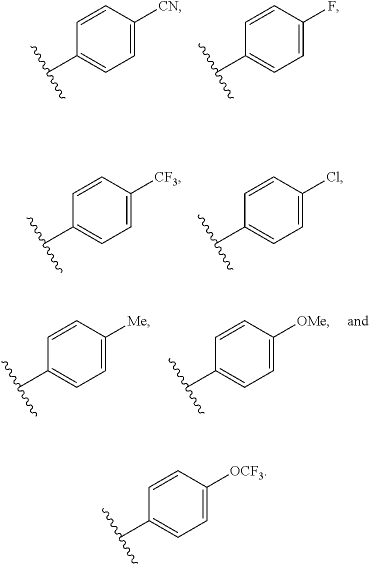

In some embodiments, R² is selected from:

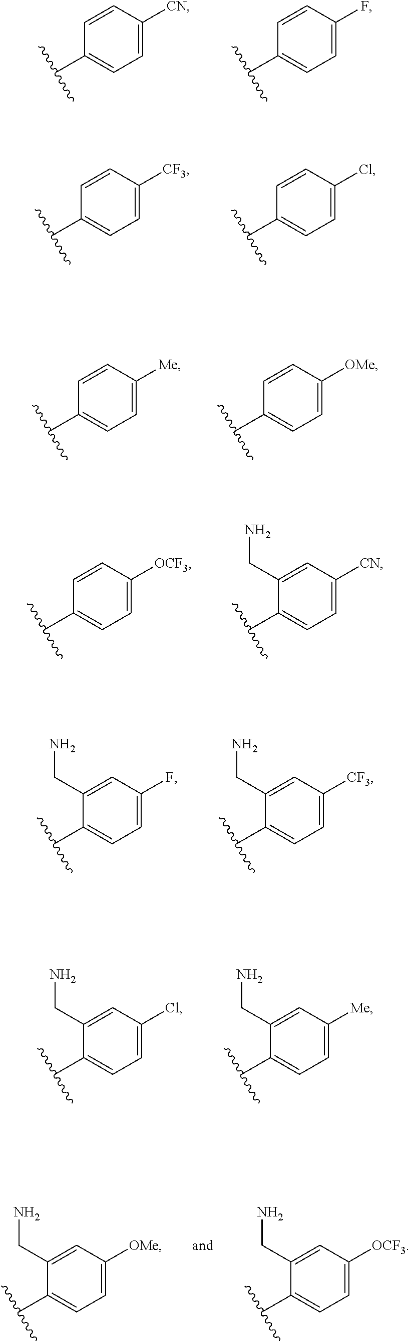

In some embodiments, R¹ is optionally substituted heterocycloalkyl. In some embodiments, R¹ is selected from:

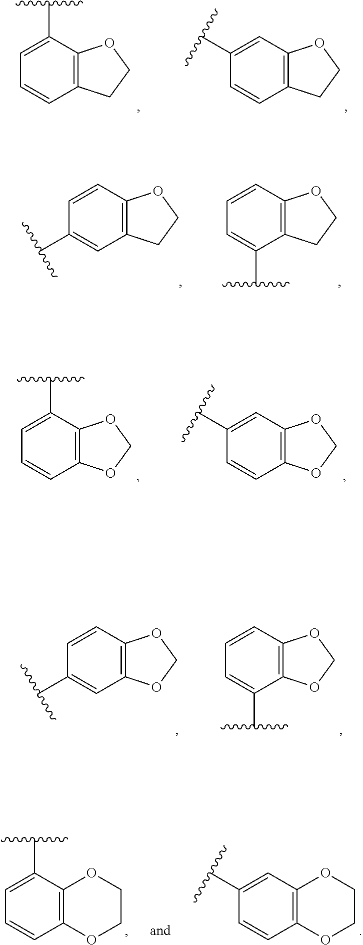

In some embodiments, R² is optionally substituted heterocycloalkyl. In some embodiments, R² is selected from:

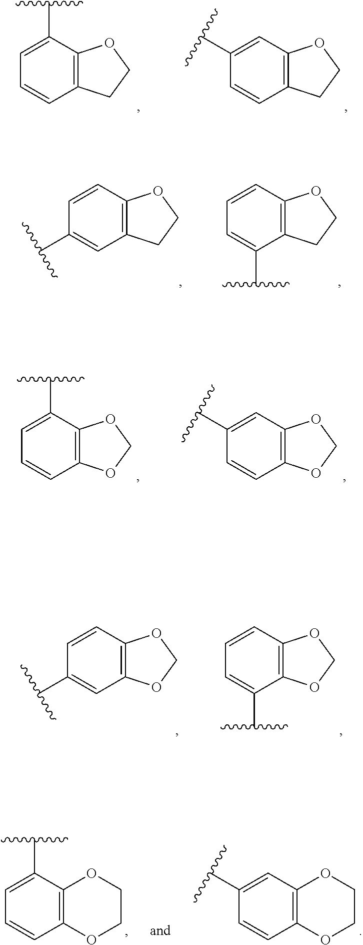

In some embodiments, a compound of Formula (Ia) is selected from:

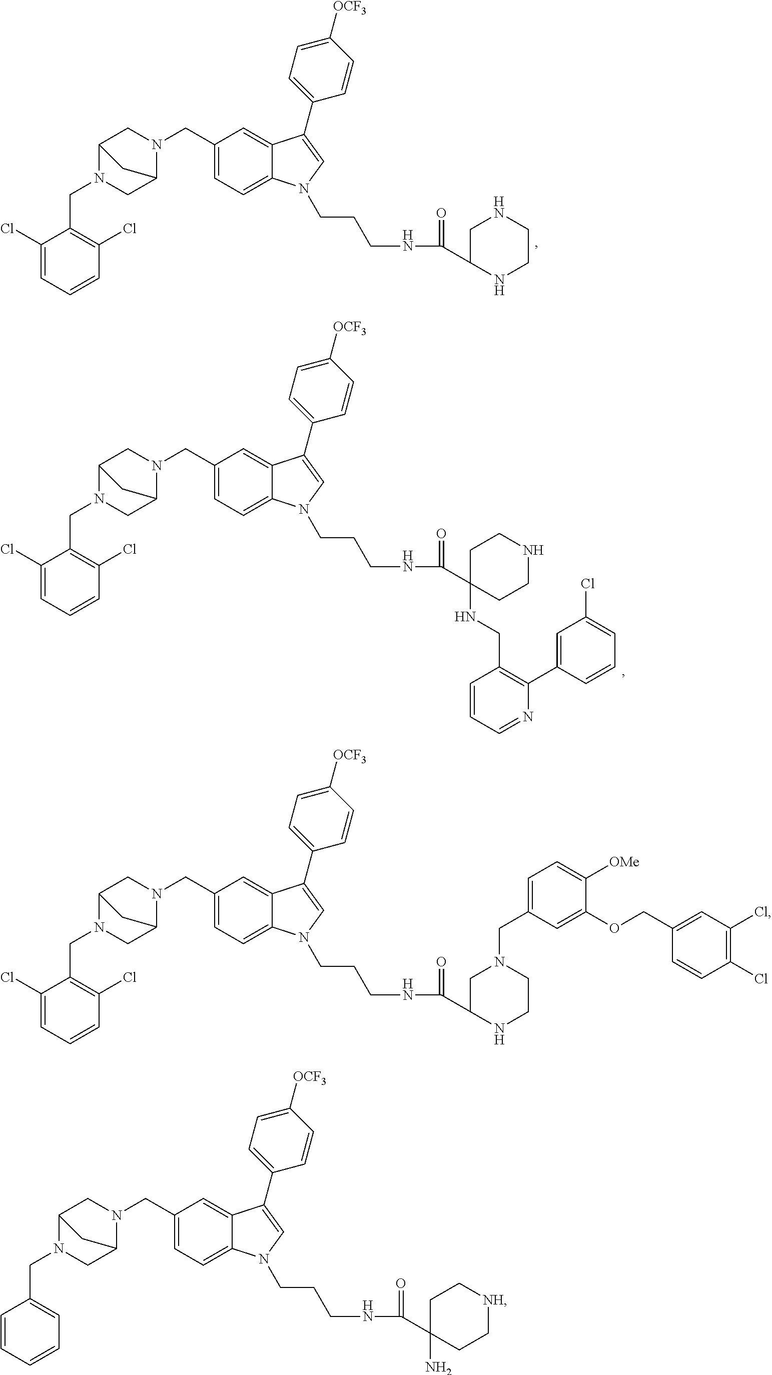 [SMILES: O=C(NCCCn1cc(-c2ccc(OC(F)(F)F)cc2)c2cc(CN3CC4CC3CN4Cc3c(Cl)cccc3Cl)ccc21)C1CNCCN1 ; O=C(NCCCn1cc(-c2ccc(OC(F)(F)F)cc2)c2cc(CN3CC4CC3CN4Cc3c(Cl)cccc3Cl)ccc21)C1(NCc2cccnc2-c2cccc(Cl)c2)CCNCC1 ; COc1ccc(CN2CCNC(C(=O)NCCCn3cc(-c4ccc(OC(F)(F)F)cc4)c4cc(CN5CC6CC5CN6Cc5c(Cl)cccc5Cl)ccc43)C2)cc1OCc1ccc(Cl)c(Cl)c1 ; NC1(C(=O)NCCCn2cc(-c3ccc(OC(F)(F)F)cc3)c3cc(CN4CC5CC4CN5Cc4ccccc4)ccc32)CCNCC1]

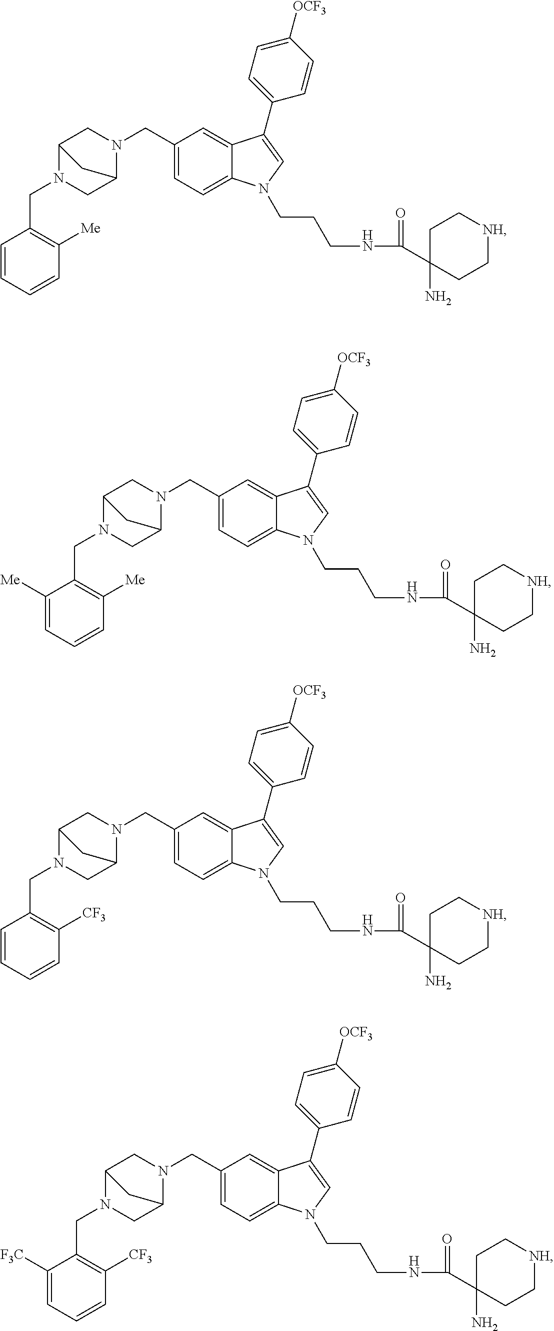 [SMILES: Cc1ccccc1CN1CC2CC1CN2Cc1ccc2c(c1)c(-c1ccc(OC(F)(F)F)cc1)cn2CCCNC(=O)C1(N)CCNCC1 ; Cc1cccc(C)c1CN1CC2CC1CN2Cc1ccc2c(c1)c(-c1ccc(OC(F)(F)F)cc1)cn2CCCNC(=O)C1(N)CCNCC1 ; NC1(C(=O)NCCCn2cc(-c3ccc(OC(F)(F)F)cc3)c3cc(CN4CC5CC4CN5Cc4ccccc4C(F)(F)F)ccc32)CCNCC1 ; NC1(C(=O)NCCCn2cc(-c3ccc(OC(F)(F)F)cc3)c3cc(CN4CC5CC4CN5Cc4c(C(F)(F)F)cccc4C(F)(F)F)ccc32)CCNCC1]

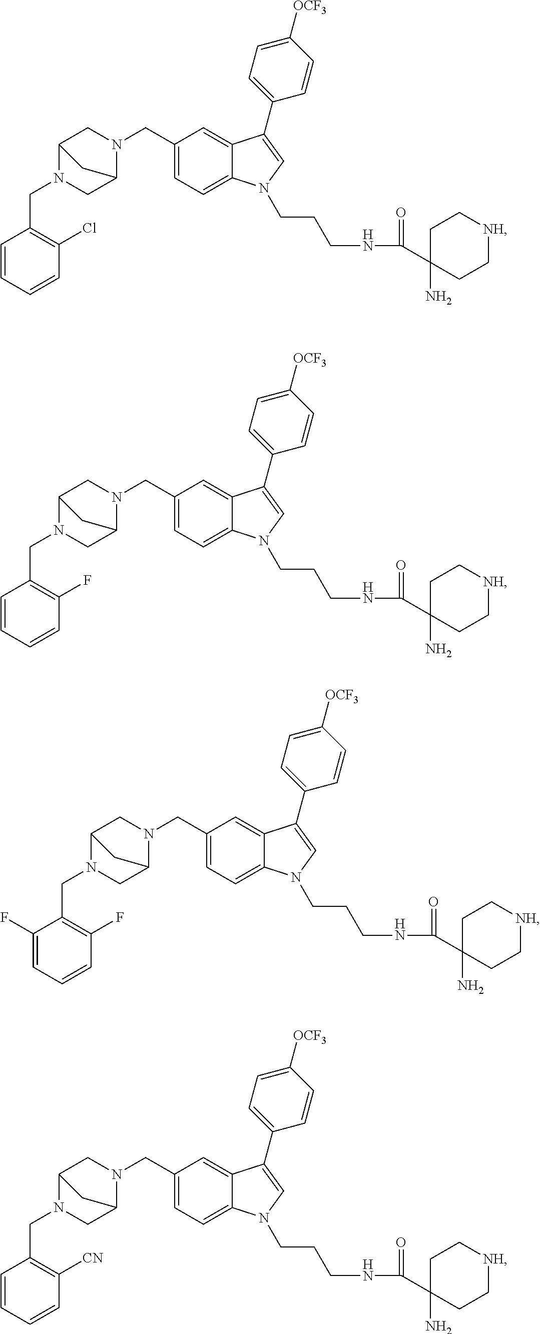 [SMILES: NC1(C(=O)NCCCn2cc(-c3ccc(OC(F)(F)F)cc3)c3cc(CN4CC5CC4CN5Cc4ccccc4Cl)ccc32)CCNCC1 ; NC1(C(=O)NCCCn2cc(-c3ccc(OC(F)(F)F)cc3)c3cc(CN4CC5CC4CN5Cc4ccccc4F)ccc32)CCNCC1 ; NC1(C(=O)NCCCn2cc(-c3ccc(OC(F)(F)F)cc3)c3cc(CN4CC5CC4CN5Cc4c(F)cccc4F)ccc32)CCNCC1 ; N#Cc1ccccc1CN1CC2CC1CN2Cc1ccc2c(c1)c(-c1ccc(OC(F)(F)F)cc1)cn2CCCNC(=O)C1(N)CCNCC1]

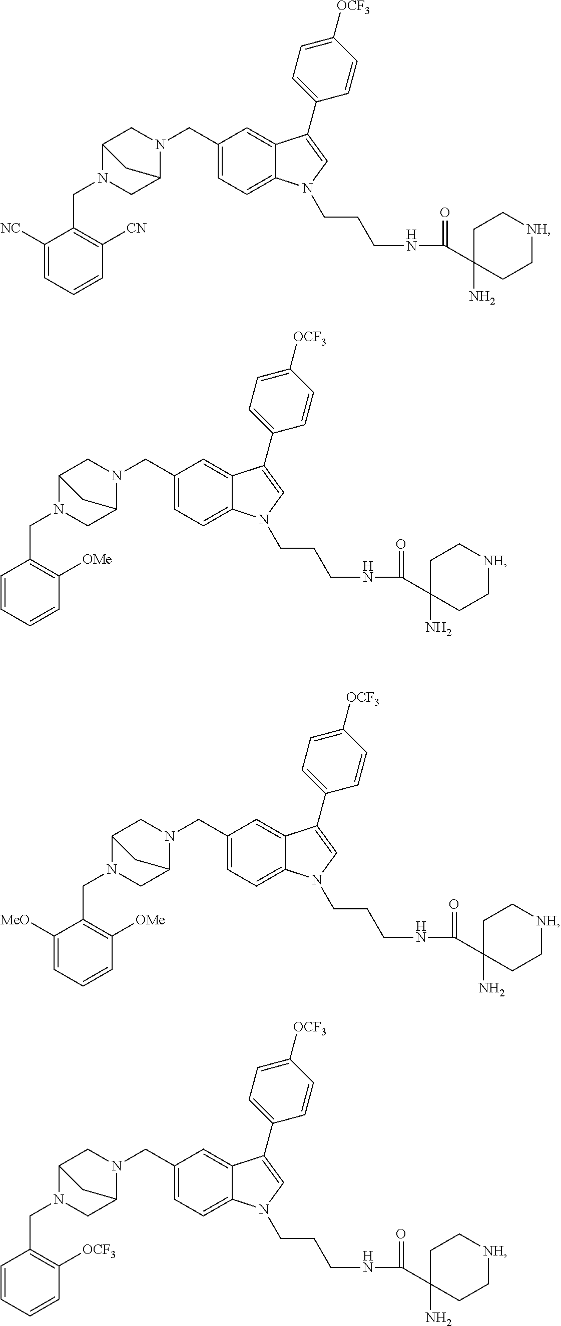 [SMILES: N#Cc1cccc(C#N)c1CN1CC2CC1CN2Cc1ccc2c(c1)c(-c1ccc(OC(F)(F)F)cc1)cn2CCCNC(=O)C1(N)CCNCC1 ; COc1ccccc1CN1CC2CC1CN2Cc1ccc2c(c1)c(-c1ccc(OC(F)(F)F)cc1)cn2CCCNC(=O)C1(N)CCNCC1 ; COc1cccc(OC)c1CN1CC2CC1CN2Cc1ccc2c(c1)c(-c1ccc(OC(F)(F)F)cc1)cn2CCCNC(=O)C1(N)CCNCC1 ; NC1(C(=O)NCCCn2cc(-c3ccc(OC(F)(F)F)cc3)c3cc(CN4CC5CC4CN5Cc4ccccc4OC(F)(F)F)ccc32)CCNCC1]

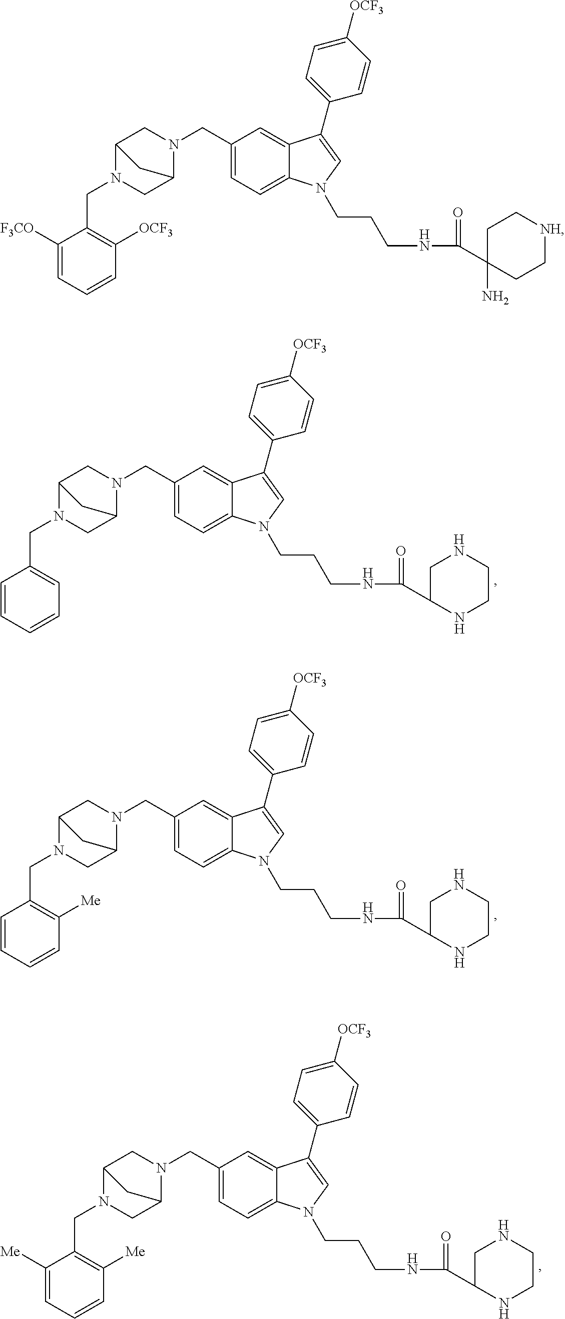 [SMILES: NC1(C(=O)NCCCn2cc(-c3ccc(OC(F)(F)F)cc3)c3cc(CN4CC5CC4CN5Cc4c(OC(F)(F)F)cccc4OC(F)(F)F)ccc32)CCNCC1 ; O=C(NCCCn1cc(-c2ccc(OC(F)(F)F)cc2)c2cc(CN3CC4CC3CN4Cc3ccccc3)ccc21)C1CNCCN1 ; Cc1ccccc1CN1CC2CC1CN2Cc1ccc2c(c1)c(-c1ccc(OC(F)(F)F)cc1)cn2CCCNC(=O)C1CNCCN1 ; Cc1cccc(C)c1CN1CC2CC1CN2Cc1ccc2c(c1)c(-c1ccc(OC(F)(F)F)cc1)cn2CCCNC(=O)C1CNCCN1]

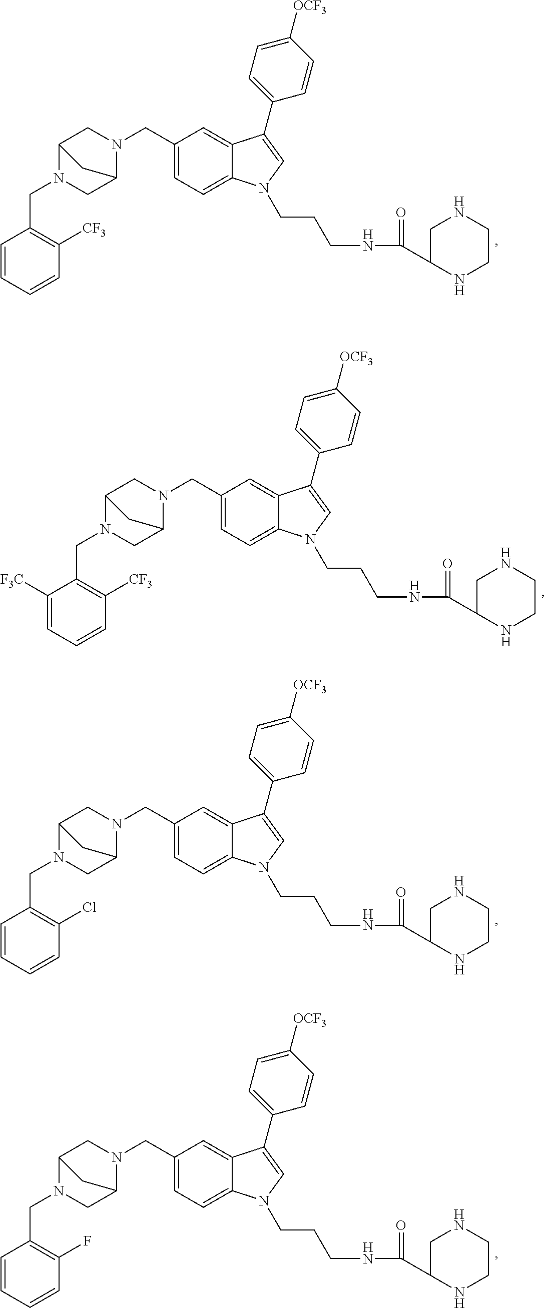 [SMILES: O=C(NCCCn1cc(-c2ccc(OC(F)(F)F)cc2)c2cc(CN3CC4CC3CN4Cc3ccccc3C(F)(F)F)ccc21)C1CNCCN1 ; O=C(NCCCn1cc(-c2ccc(OC(F)(F)F)cc2)c2cc(CN3CC4CC3CN4Cc3c(C(F)(F)F)cccc3C(F)(F)F)ccc21)C1CNCCN1 ; O=C(NCCCn1cc(-c2ccc(OC(F)(F)F)cc2)c2cc(CN3CC4CC3CN4Cc3ccccc3Cl)ccc21)C1CNCCN1 ; O=C(NCCCn1cc(-c2ccc(OC(F)(F)F)cc2)c2cc(CN3CC4CC3CN4Cc3ccccc3F)ccc21)C1CNCCN1]

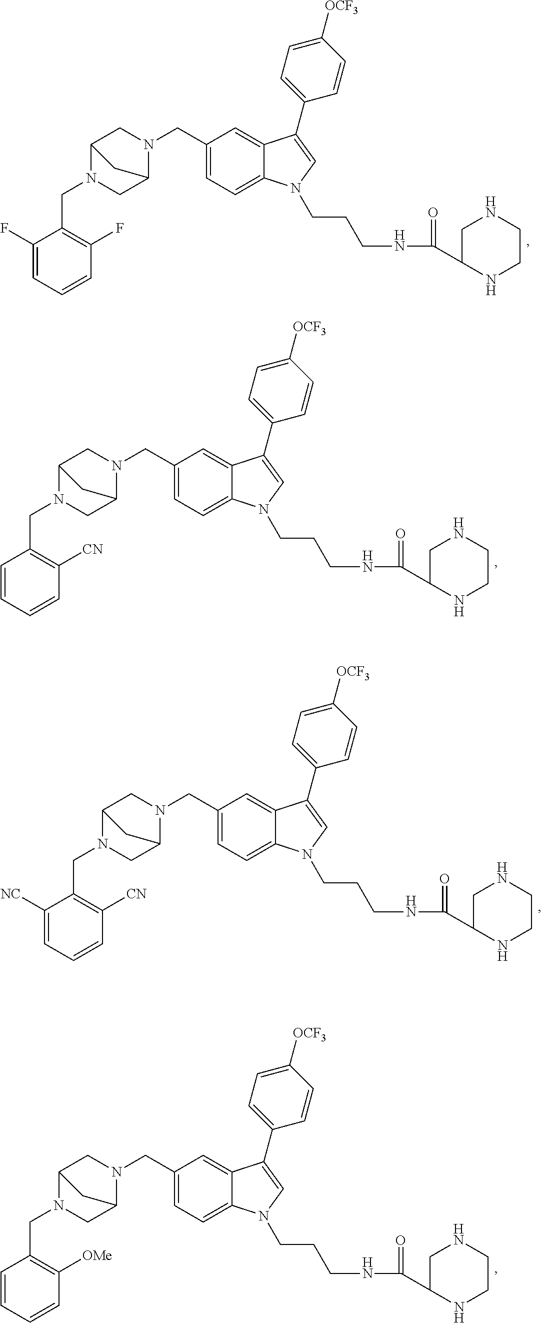 [SMILES: O=C(NCCCn1cc(-c2ccc(OC(F)(F)F)cc2)c2cc(CN3CC4CC3CN4Cc3c(F)cccc3F)ccc21)C1CNCCN1 ; N#Cc1ccccc1CN1CC2CC1CN2Cc1ccc2c(c1)c(-c1ccc(OC(F)(F)F)cc1)cn2CCCNC(=O)C1CNCCN1 ; N#Cc1cccc(C#N)c1CN1CC2CC1CN2Cc1ccc2c(c1)c(-c1ccc(OC(F)(F)F)cc1)cn2CCCNC(=O)C1CNCCN1 ; COc1ccccc1CN1CC2CC1CN2Cc1ccc2c(c1)c(-c1ccc(OC(F)(F)F)cc1)cn2CCCNC(=O)C1CNCCN1]

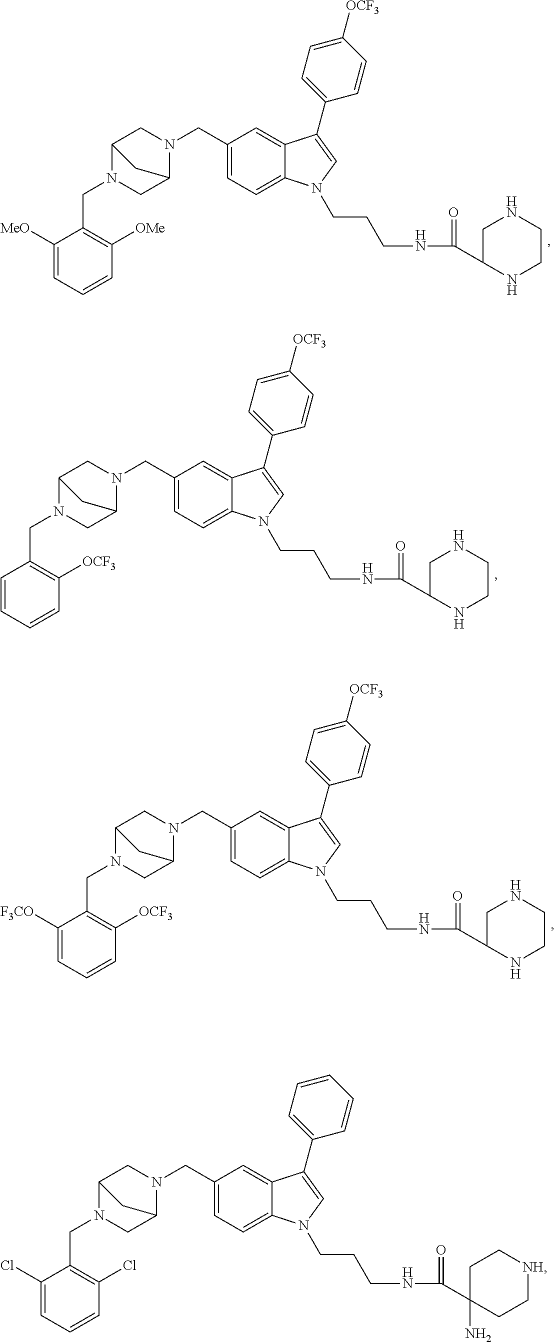 [SMILES: COc1cccc(OC)c1CN1CC2CC1CN2Cc1ccc2c(c1)c(-c1ccc(OC(F)(F)F)cc1)cn2CCCNC(=O)C1CNCCN1 ; O=C(NCCCn1cc(-c2ccc(OC(F)(F)F)cc2)c2cc(CN3CC4CC3CN4Cc3ccccc3OC(F)(F)F)ccc21)C1CNCCN1 ; O=C(NCCCn1cc(-c2ccc(OC(F)(F)F)cc2)c2cc(CN3CC4CC3CN4Cc3c(OC(F)(F)F)cccc3OC(F)(F)F)ccc21)C1CNCCN1 ; NC1(C(=O)NCCCn2cc(-c3ccccc3)c3cc(CN4CC5CC4CN5Cc4c(Cl)cccc4Cl)ccc32)CCNCC1]

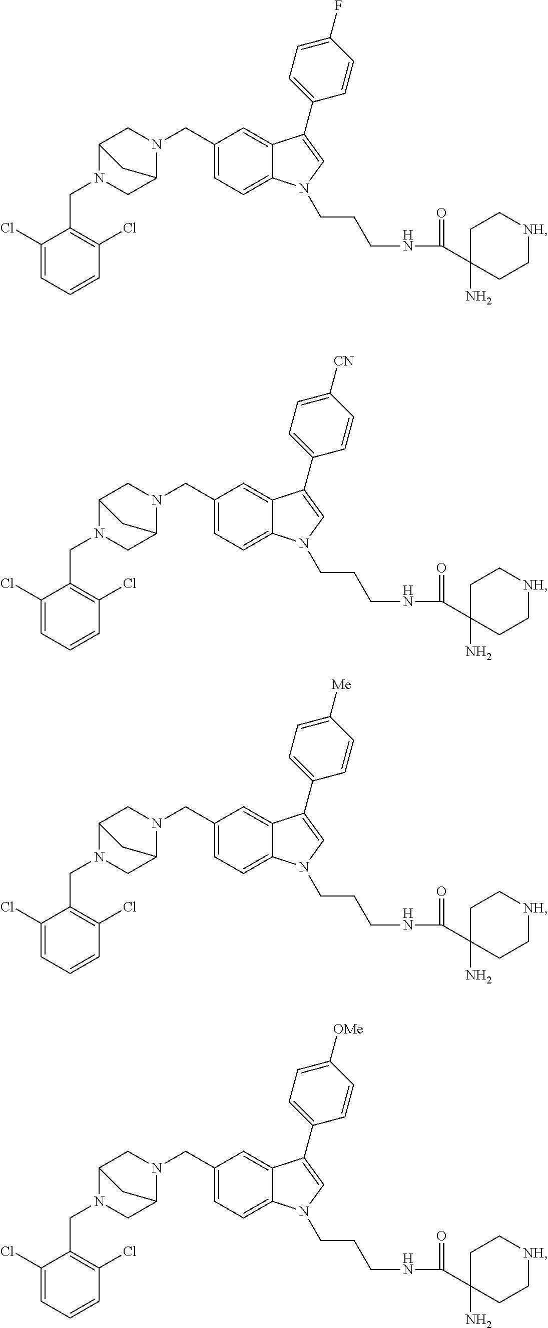 [SMILES: NC1(C(=O)NCCCn2cc(-c3ccc(F)cc3)c3cc(CN4CC5CC4CN5Cc4c(Cl)cccc4Cl)ccc32)CCNCC1 ; N#Cc1ccc(-c2cn(CCCNC(=O)C3(N)CCNCC3)c3ccc(CN4CC5CC4CN5Cc4c(Cl)cccc4Cl)cc23)cc1 ; Cc1ccc(-c2cn(CCCNC(=O)C3(N)CCNCC3)c3ccc(CN4CC5CC4CN5Cc4c(Cl)cccc4Cl)cc23)cc1 ; COc1ccc(-c2cn(CCCNC(=O)C3(N)CCNCC3)c3ccc(CN4CC5CC4CN5Cc4c(Cl)cccc4Cl)cc23)cc1]

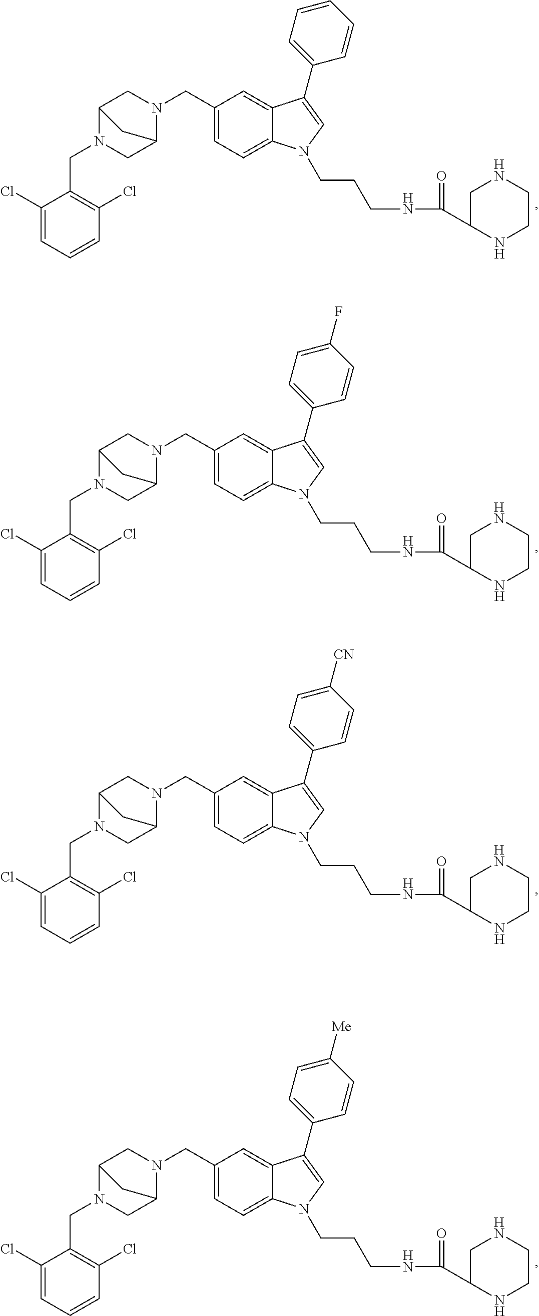 [SMILES: O=C(NCCCn1cc(-c2ccccc2)c2cc(CN3CC4CC3CN4Cc3c(Cl)cccc3Cl)ccc21)C1CNCCN1 ; O=C(NCCCn1cc(-c2ccc(F)cc2)c2cc(CN3CC4CC3CN4Cc3c(Cl)cccc3Cl)ccc21)C1CNCCN1 ; N#Cc1ccc(-c2cn(CCCNC(=O)C3CNCCN3)c3ccc(CN4CC5CC4CN5Cc4c(Cl)cccc4Cl)cc23)cc1 ; Cc1ccc(-c2cn(CCCNC(=O)C3CNCCN3)c3ccc(CN4CC5CC4CN5Cc4c(Cl)cccc4Cl)cc23)cc1]

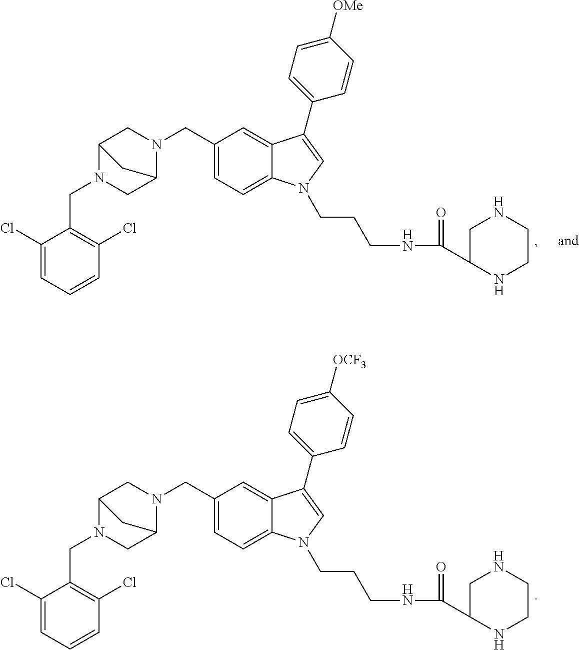 [SMILES: COc1ccc(-c2cn(CCCNC(=O)C3CNCCN3)c3ccc(CN4CC5CC4CN5Cc4c(Cl)cccc4Cl)cc23)cc1 ; O=C(NCCCn1cc(-c2ccc(OC(F)(F)F)cc2)c2cc(CN3CC4CC3CN4Cc3c(Cl)cccc3Cl)ccc21)C1CNCCN1]

In some embodiments, compounds of Formula (Ia) include, but are not limited to, those of Formula (If) as described in Table 2.

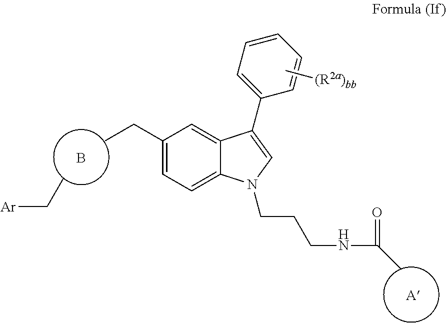

TABLE 2

| Entry | 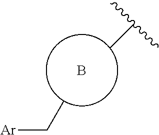 |
|---|---|
| 1 | 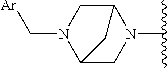 |
| 2 | 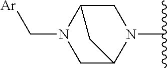 |
| 3 | 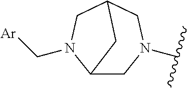 |
| 4 | 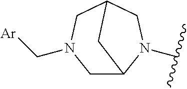 |
| 5 | 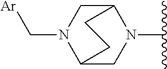 |
| 6 | 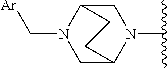 |
| 7 | 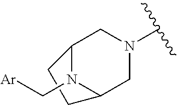 |
| 8 | 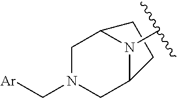 |
| 9 | 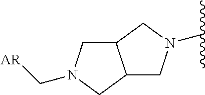 |
| 10 | 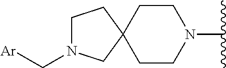 |
| 11 | 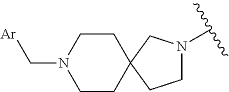 |
| 12 | 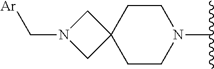 |
| 13 | 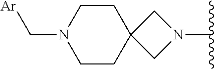 |
| 14 | 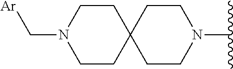 |
| 15 | 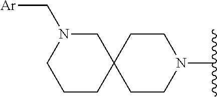 |
| 16 | 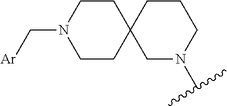 |
| 17 | 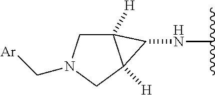 |
| 18 | 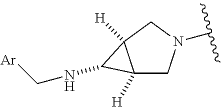 |

For compounds of Formula (If), each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, R^{2a} is —CH₂NH₂. In some embodiments, bb is 0. In some embodiments bb is 1. In some embodiments, R^{2a} is halogen and bb is 1. In some embodiments, R^{2a} is —OCF₃ and bb is 1. In some embodiments, Ar is unsubstituted phenyl. In some embodiments, Ar is a substituted phenyl. In some embodiments, Ar is selected from:

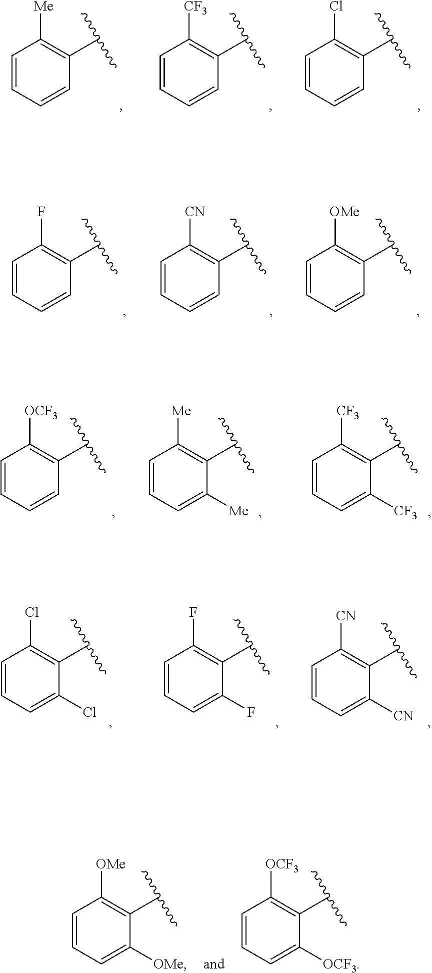

In some embodiments,

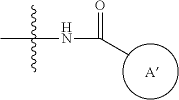

is selected from

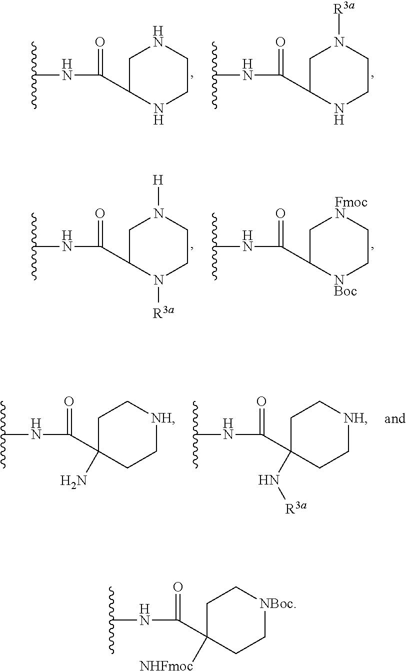

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

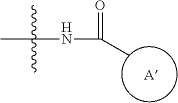

is selected from

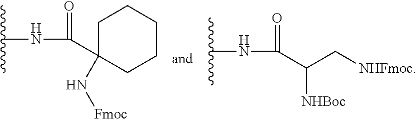

In some embodiments, compounds of Formula (Ia) include, but are not limited to, those of Formula (Ig) as described in Table 3.

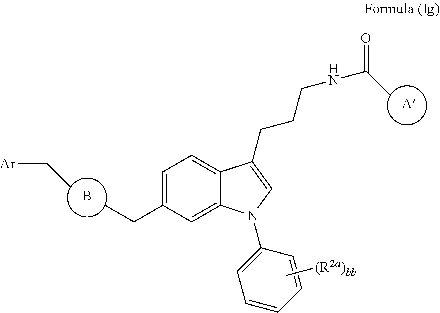

TABLE 3

| Entry | 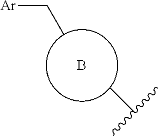 |
|---|---|
| 1 | 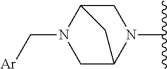 |
| 2 | 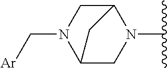 |
| 3 | 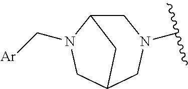 |
| 4 | 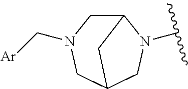 |
| 5 | 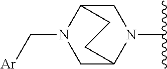 |
| 6 | 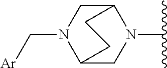 |
| 7 | 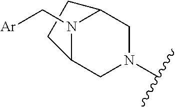 |
| 8 | 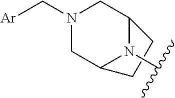 |
| 9 | 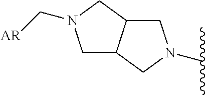 |
| 10 | 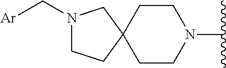 |
| 11 | 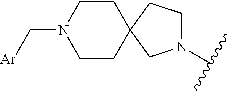 |
| 12 | 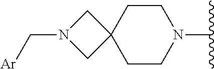 |
| 13 | 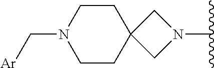 |
| 14 | 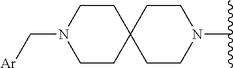 |
| 15 | 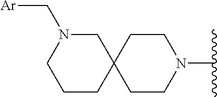 |
| 16 | 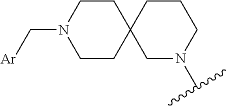 |
| 17 | 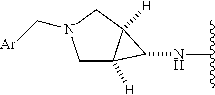 |
| 18 | 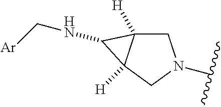 |

For compounds of Formula (Ig), each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, R^{2a} is —CH₂NH₂. In some embodiments, bb is 0. In some embodiments bb is 1. In some embodiments, R^{2a} is halogen and bb is 1. In some embodiments, R^{2a} is —OCF₃ and bb is 1. In some embodiments, Ar is unsubstituted phenyl. In some embodiments, Ar is a substituted phenyl. In some embodiments, Ar is selected from:

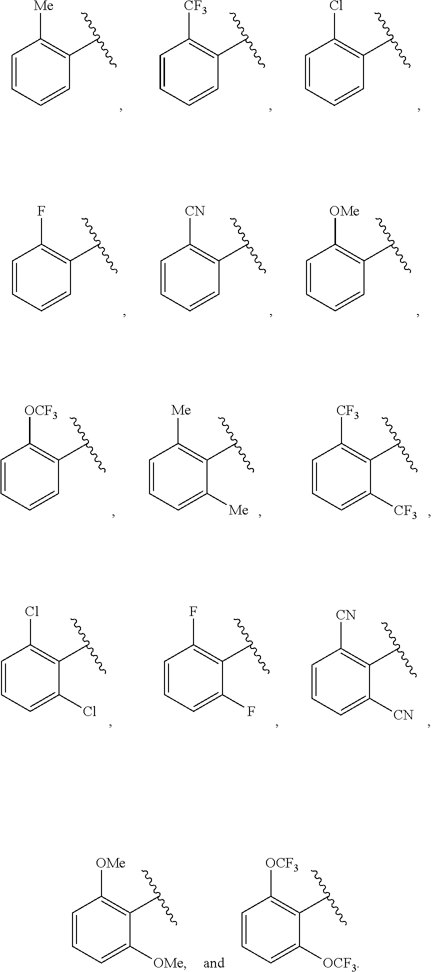

In some embodiments,

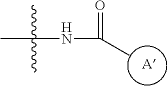

is selected from

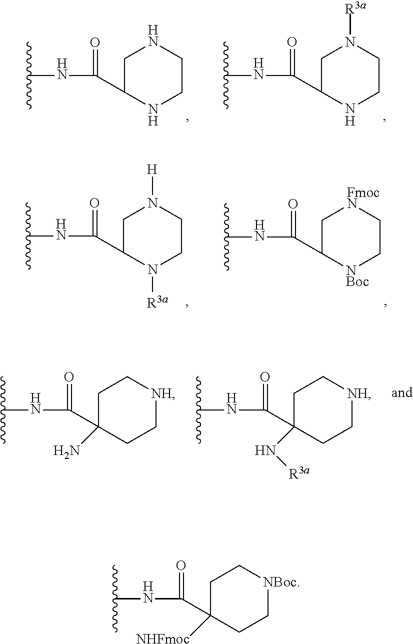

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

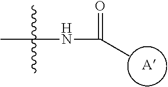

is selected from

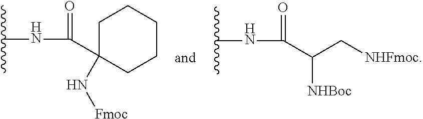

In some embodiments, compounds of Formula (Ig) include compounds wherein

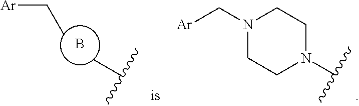

In some embodiments, a compound of Formula (Ia) is selected from any one of the compounds from the following table:

TABLE 4

| Compound | | |
|---|---|---|
| Ref. | Compound | Chemical Name |
| C45 | 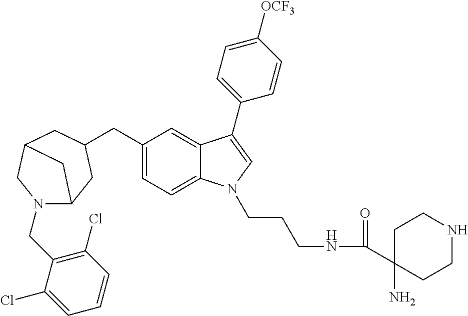 [SMILES: NC1(C(=O)NCCCn2cc(-c3ccc(OC(F)(F)F)cc3)c3cc(CC4CC5CC(C4)N(Cc4c(Cl)cccc4Cl)C5)ccc32)CCNCC1] | 4-amino-N-(3-(5-((6-(2,6- dichlorobenzyl)-3,6- diazabicyclo[3.2.1]octan-3- yl)methyl)-3-(4- (trifluoromethoxy)phenyl)- 1H-indol-1- yl)propyl)piperidine-4- carboxamide |
| C56 | 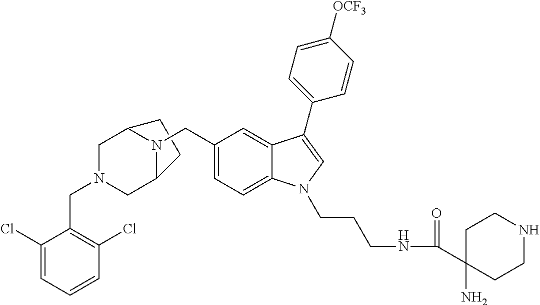 [SMILES: NC1(C(=O)NCCCn2cc(-c3ccc(OC(F)(F)F)cc3)c3cc(CN4C5CCC4CN(Cc4c(Cl)cccc4Cl)C5)ccc32)CCNCC1] | 4-amino-N-(3-(5-((3-(2,6- dichlorobenzyl)-3,8- diazabicyclo[3.2.1]octan-8- yl)methyl)-3-(4- (trifluoromethoxy)phenyl)- 1H-indol-1- yl)propyl)piperidine-4- carboxamide |
| C61 | 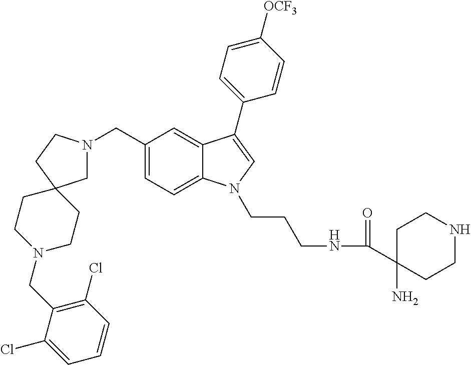 [SMILES: NC1(C(=O)NCCCn2cc(-c3ccc(OC(F)(F)F)cc3)c3cc(CN4CCC5(CCN(Cc6c(Cl)cccc6Cl)CC5)C4)ccc32)CCNCC1] | 4-amino-N-(3-(5-((8-(2,6- dichlorobenzyl)-2,8- diazaspiro[4.5]decan-2- yl)methyl)-3-(4- (trifluoromethoxy)phenyl)- 1H-indol-1- yl)propyl)piperidine-4- carboxamide |
| C65 | 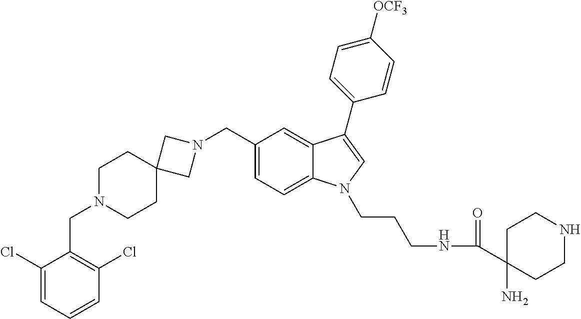 [SMILES: NC1(C(=O)NCCCn2cc(-c3ccc(OC(F)(F)F)cc3)c3cc(CN4CC5(CCN(Cc6c(Cl)cccc6Cl)CC5)C4)ccc32)CCNCC1] | 4-amino-N-(3-(5-((7-(2,6- dichlorobenzyl)-2,7- diazaspiro[3.5]nonan-2- yl)methyl)-3-(4- (trifluoromethoxy)phenyl)- 1H-indol-1- yl)propyl)piperidine-4- carboxamide |
| C67 | 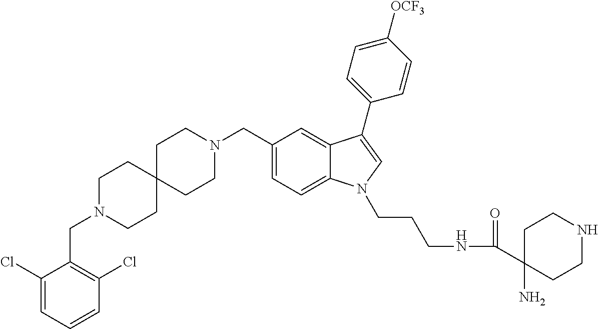 [SMILES: NC1(C(=O)NCCCn2cc(-c3ccc(OC(F)(F)F)cc3)c3cc(CN4CCC5(CC4)CCN(Cc4c(Cl)cccc4Cl)CC5)ccc32)CCNCC1] | 4-amino-N-(3-(5-((9-(2,6- dichlorobenzyl)-3,9- diazaspiro[5.5]undecan-3- yl)methyl)-3-(4- (trifluoromethoxy)phenyl)- 1H-indol-1- yl)propyl)piperidine-4- carboxamide |
| C68 | 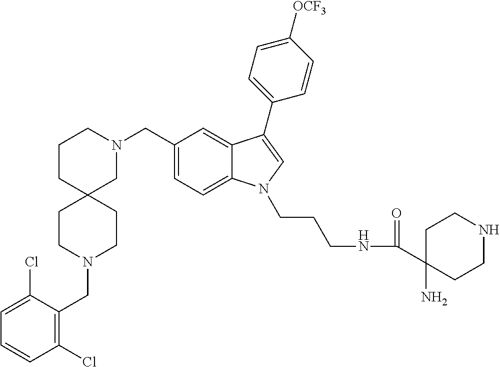 [SMILES: NC1(C(=O)NCCCn2cc(-c3ccc(OC(F)(F)F)cc3)c3cc(CN4CCCC5(CCN(Cc6c(Cl)cccc6Cl)CC5)C4)ccc32)CCNCC1] | 4-amino-N-(3-(5-((9-(2,6- dichlorobenzyl)-2,9- diazaspiro[5.5]undecan-2- yl)methyl)-3-(4- (trifluoromethoxy)phenyl)- 1H-indol-1- yl)propyl)piperidine-4- carboxamide |
| C69 | 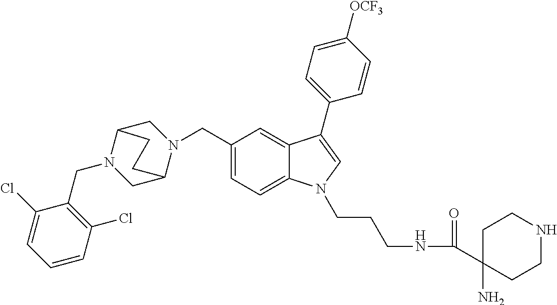 [SMILES: NC1(C(=O)NCCCn2cc(-c3ccc(OC(F)(F)F)cc3)c3cc(CN4CC5CCC4CN5Cc4c(Cl)cccc4Cl)ccc32)CCNCC1] | 4-amino-N-(3-(5-((5-(2,6- dichlorobenzyl)-2,5- diazabicyclo[2.2.2]octan-2- yl)methyl)-3-(4- (trifluoromethoxy)phenyl)- 1H-indol-1- yl)propyl)piperidine-4- carboxamide |
| C70 | 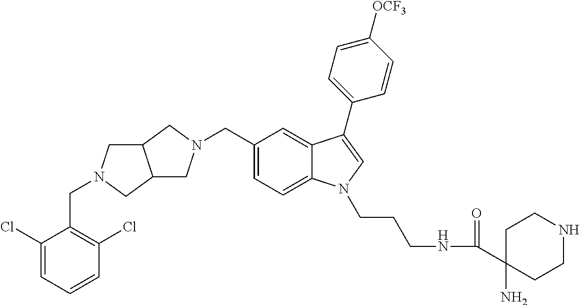 [SMILES: NC1(C(=O)NCCCn2cc(-c3ccc(OC(F)(F)F)cc3)c3cc(CN4CC5CN(Cc6c(Cl)cccc6Cl)CC5C4)ccc32)CCNCC1] | 4-amino-N-(3-(5-((5-(2,6- dichlorobenzyl) hexahydropyrrolo [3,4-c]pyrrol-2(1H)- yl)methyl)-3-(4- (trifluoromethoxy)phenyl)- 1H-indol-1- yl)propyl)piperidine-4- carboxamide |
| C76 | 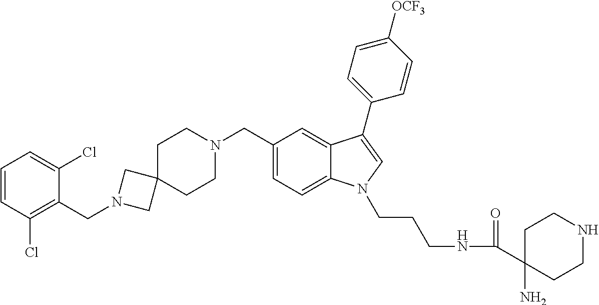 [SMILES: NC1(C(=O)NCCCn2cc(-c3ccc(OC(F)(F)F)cc3)c3cc(CN4CCC5(CC4)CN(Cc4c(Cl)cccc4Cl)C5)ccc32)CCNCC1] | 4-amino-N-(3-(5-((2-(2,6- dichlorobenzyl)-2,7- diazaspiro[3.5]nonan-7- yl)methyl)-3-(4- (trifluoromethoxy)phenyl)- 1H-indol-1- yl)propyl)piperidine-4- carboxamide |
| C77 | 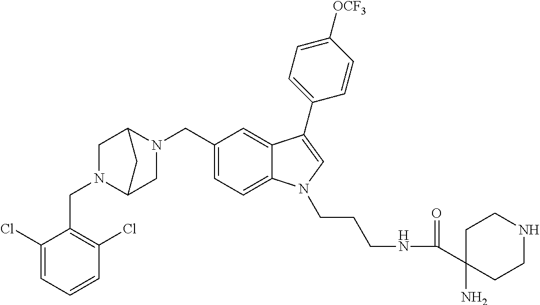 [SMILES: NC1(C(=O)NCCCn2cc(-c3ccc(OC(F)(F)F)cc3)c3cc(CN4CC5CC4CN5Cc4c(Cl)cccc4Cl)ccc32)CCNCC1] | 4-amino-N-(3-(5-((5-(2,6- dichlorobenzyl)-2,5- diazabicyclo[2.2.1]heptan-2- yl)methyl)-3-(4- (trifluoromethoxy)phenyl)- 1H-indol-1- yl)propyl)piperidine-4- carboxamide |
| C78 | 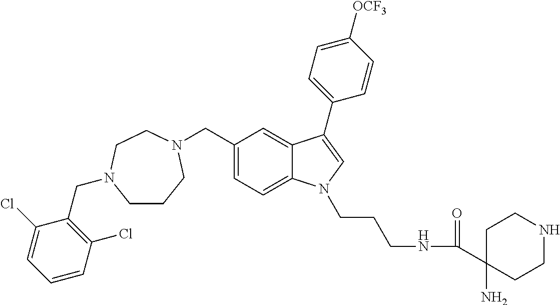 [SMILES: NC1(C(=O)NCCCn2cc(-c3ccc(OC(F)(F)F)cc3)c3cc(CN4CCCN(Cc5c(Cl)cccc5Cl)CC4)ccc32)CCNCC1] | 4-amino-N-(3-(5-((4-(2,6- dichlorobenzyl)-1,4-diazepan- 1-yl)methyl)-3-(4- (trifluoromethoxy)phenyl)- 1H-indol-1- yl)propyl)piperidine-4- carboxamide |
| C79 | 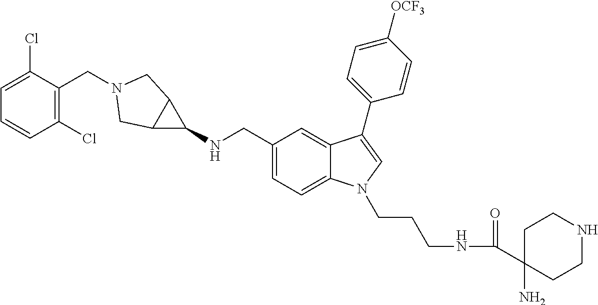 | 4-amino-N-(3-(5-((3-(2,6- dichlorobenzyl)-3- azabicyclo[3.1.0]hexan-6- ylamino)methyl)-3-(4- (trifluoromethoxy)phenyl)- 1H-indol-1- yl)propyl)piperidine-4- carboxamide |
| C83 | 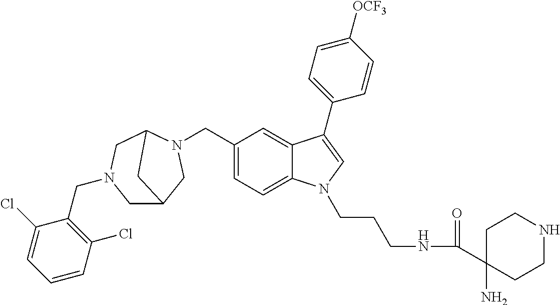 [SMILES: NC1(C(=O)NCCCn2cc(-c3ccc(OC(F)(F)F)cc3)c3cc(CN4CC5CC4CN(Cc4c(Cl)cccc4Cl)C5)ccc32)CCNCC1] | 4-amino-N-(3-(5-((3-(2,6- dichlorobenzyl)-3,6- diazabicyclo[3.2.1]octan-6- yl)methyl)-3-(4- (trifluoromethoxy)phenyl)- 1H-indol-1- yl)propyl)piperidine-4- carboxamide |
| C85 | 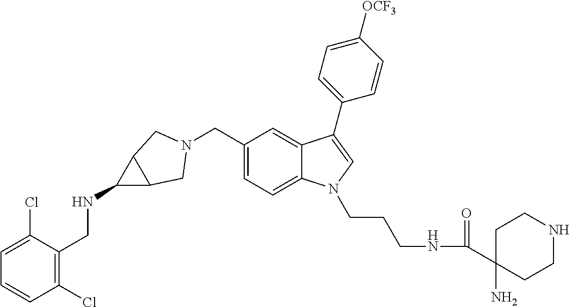 | 4-amino-N-(3-(5-((6-(2,6- dichlorobenzylamino)-3- azabicyclo[3.1.0]hexan-3- yl)methyl)-3-(4- (trifluoromethoxy)phenyl)- 1H-indol-1- yl)propyl)piperidine-4- carboxamide |
| C86 | 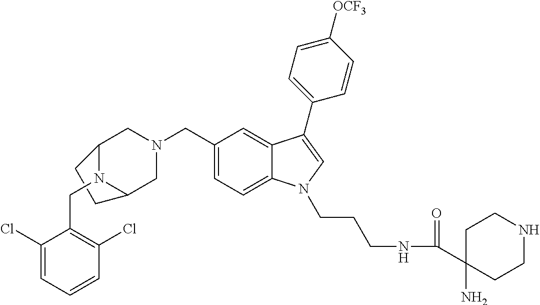 [SMILES: NC1(C(=O)NCCCn2cc(-c3ccc(OC(F)(F)F)cc3)c3cc(CN4CC5CCC(C4)N5Cc4c(Cl)cccc4Cl)ccc32)CCNCC1] | 4-amino-N-(3-(5-((8-(2,6- dichlorobenzyl)-3,8- diazabicyclo[3.2.1]octan-3- yl)methyl)-3-(4- (trifluoromethoxy)phenyl)- 1H-indol-1- yl)propyl)piperidine-4- carboxamide |

In one aspect provided herein is a compound of Formula (IIa), or a pharmaceutically acceptable salt, or solvate thereof:

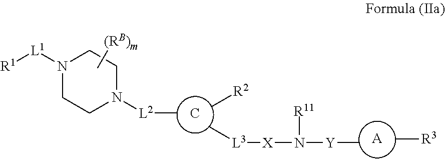

wherein,

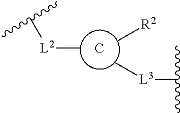

is a bicyclic heteroaryl that is selected from the following structures:

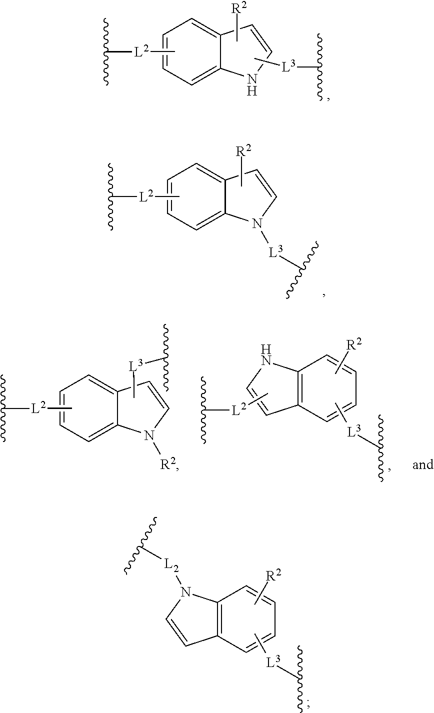

L¹ and L² are each independently an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is an optionally substituted aryl, optionally substituted heterocycloalkyl, or optionally substituted heteroaryl;
R² is an optionally substituted aryl, optionally substituted heterocycloalkyl, or optionally substituted heteroaryl;
each R^{B} is independently optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, a substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)— or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N; wherein if Ring A is substituted, then Ring A is substituted with at least one R^{A};
each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring, and
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring,
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); and
each m is independently 0, 1, 2, 3, or 4.

In some embodiments,

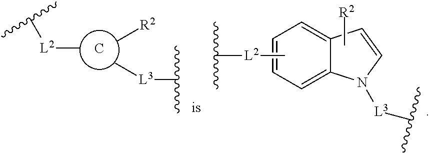

In some embodiments,

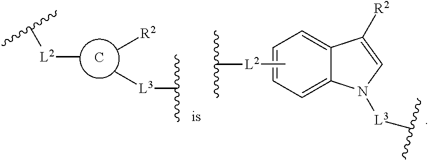

In some embodiments, a compound of Formula (IIa) has the following structure of Formula (IIb), or a pharmaceutically acceptable salt, or solvate thereof:

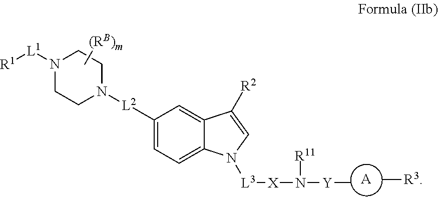

In some embodiments, a compound of Formula (IIa) has the following structure of Formula (IIc), or a pharmaceutically acceptable salt, or solvate thereof:

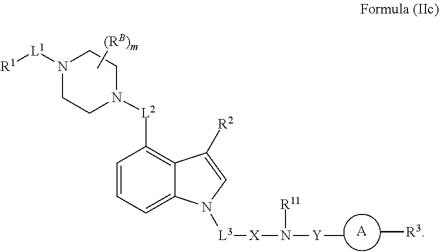

In some embodiments,

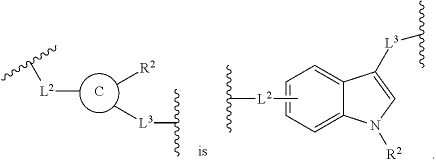

In some embodiments,

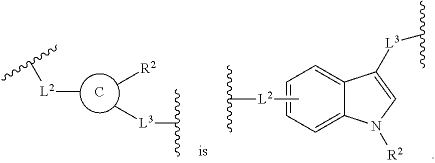

In some embodiments, a compound of Formula (IIa) has the following structure of Formula (IId), or a pharmaceutically acceptable salt, or solvate thereof:

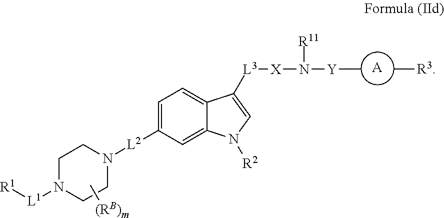

In some embodiments, a compound of Formula (IIa) has the following structure of Formula (IIe), or a pharmaceutically acceptable salt, or solvate thereof:

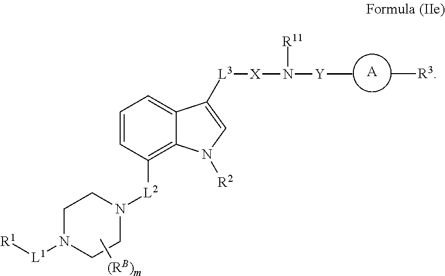

In some embodiments,

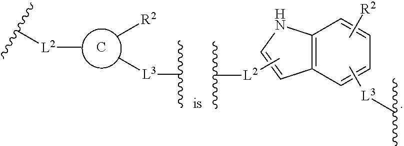

In some embodiments,

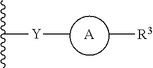

is selected from the following:

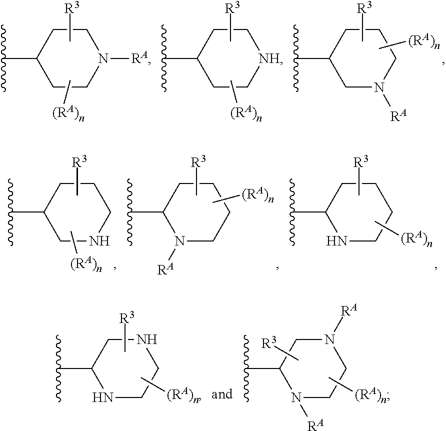

and each n is independently 0, 1, 2, 3, or 4.

In some embodiments,

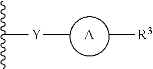

is selected from the following:

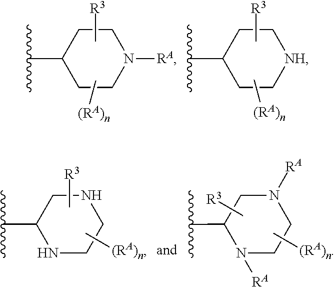

In some embodiments,

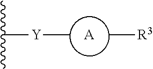

is selected from the following:

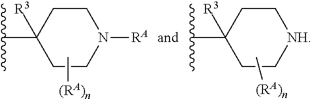

In some embodiments, L¹ is —CH₂—, C(═O), O, S, S(═O), S(═O)₂, or NR⁴. In some embodiments, L¹ is —CH_{2—}. In some embodiments, L² is —CH₂—, C(═O), O, S, S(═O), S(═O)₂, or NR⁴. In some embodiments, L² is —CH₂—.

In some embodiments, L³ is an optionally substituted C₁-C₆heteroalkylene, a substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene). In some embodiments, L³ is a substituted C₁-C₅alkylene. In some embodiments, L³ is:

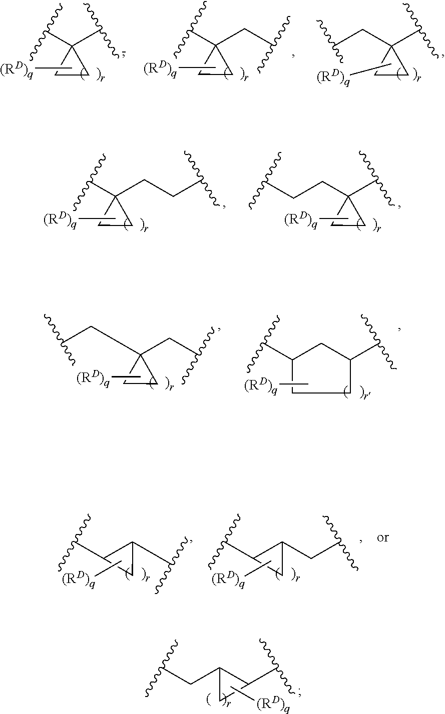

each q is independently 0, 1, 2, 3, or 4;

r is 1, 2, 3, 4, or 5; and

r′ is 1 or 2.

In some embodiments, X is —CH₂— or C(═O). In some embodiments, X is —CH₂—.

In some embodiments, X is:

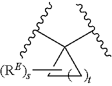

each s is independently 0, 1, 2, 3, or 4; and

t is 1, 2, 3, 4, or 5.

In some embodiments, X is:

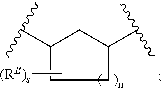

each s is independently 0, 1, 2, 3, or 4; and

u is 0, 1, or 2.

In some embodiments, Y is —CH₂— or C(═O). In some embodiments, Y is C(═O). In some embodiments, R¹¹ is hydrogen.

In some embodiments, R³ is H. In some embodiments, R³ is CH₂N(R⁹)(R¹⁰). In some embodiments, R³ is N(R⁹)(R¹⁰).

In some embodiments, R⁹ and R¹⁰ are each H. In some embodiments, R⁹ is H and R¹⁰ is —C₁-C₄alkylene-(optionally substituted phenyl) or —C₁-C₄alkylene-(optionally substituted heteroaryl). In some embodiments, R⁹ is H and R¹⁰ is —CH₂-(optionally substituted phenyl). In some embodiments, R⁹ is H and R¹⁰ is —CH₂-(optionally substituted heteroaryl).

In some embodiments, R³ is CH₂N(R⁹)(R¹⁰); and R⁹ and R¹⁰ are each H. In some embodiments, R³ is CH₂N(R⁹)(R¹⁰); and R⁹ is H and R¹⁰ is —C₁-C₄alkylene-(optionally substituted phenyl) or —C₁-C₄alkylene-(optionally substituted heteroaryl). In some embodiments, R³ is CH₂N(R⁹)(R¹⁰); and R⁹ is H and R¹⁰ is —C₁-C₄alkylene-(optionally substituted phenyl). In some embodiments, R³ is CH₂N(R⁹)(R¹⁰); and R⁹ is H and R¹⁰ is —C₁-C₄alkylene-(optionally substituted heteroaryl). In some embodiments, R³ is N(R⁹)(R¹⁰); and R⁹ and R¹⁰ are each H. In some embodiments, R³ is N(R⁹)(R¹⁰); and R⁹ is H and R¹⁰ is —C₁-C₄alkylene-(optionally substituted phenyl) or —C₁-C₄alkylene-(optionally substituted heteroaryl). In some embodiments, R³ is N(R⁹)(R¹⁰); and R⁹ is H and R¹⁰ is —C₁-C₄alkylene-(optionally substituted phenyl). In some embodiments, R³ is N(R⁹)(R¹⁰); and R⁹ is H and R¹⁰ is —C₁-C₄alkylene-(optionally substituted heteroaryl).

In some embodiments, R¹ is an unsubstituted phenyl. In some embodiments, R¹ is a substituted phenyl. In some embodiments, R¹ is selected from:

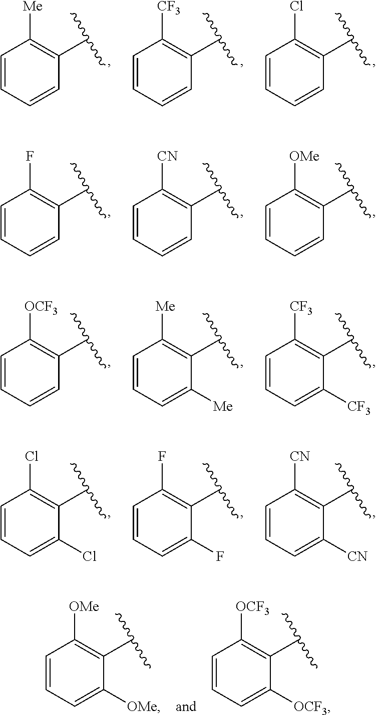

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

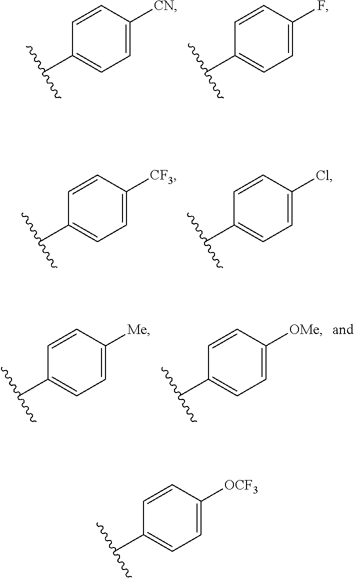

In some embodiments, R² is selected from:

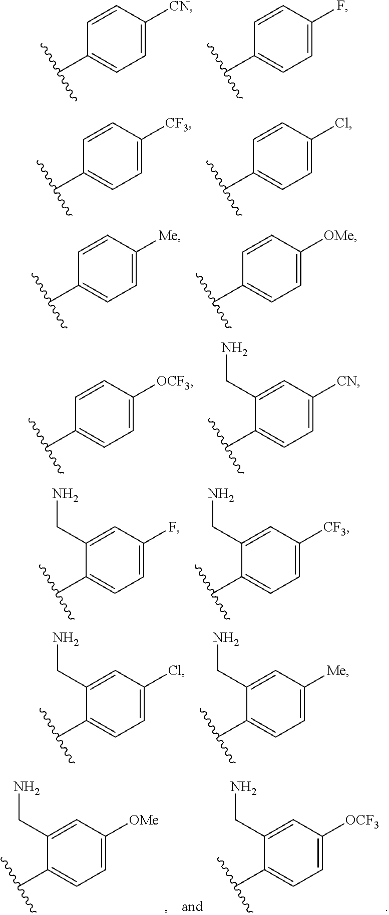

In some embodiments, R¹ is optionally substituted heterocycloalkyl. In some embodiments, R¹ is selected from:

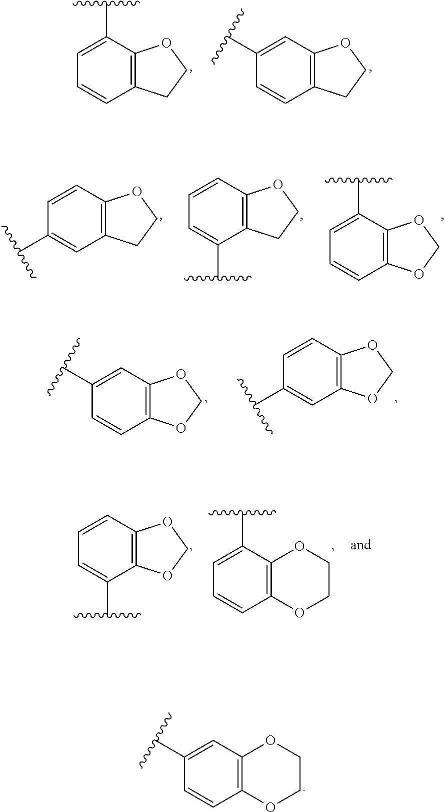

In some embodiments, R² is optionally substituted heterocycloalkyl. In some embodiments, R² is selected from:

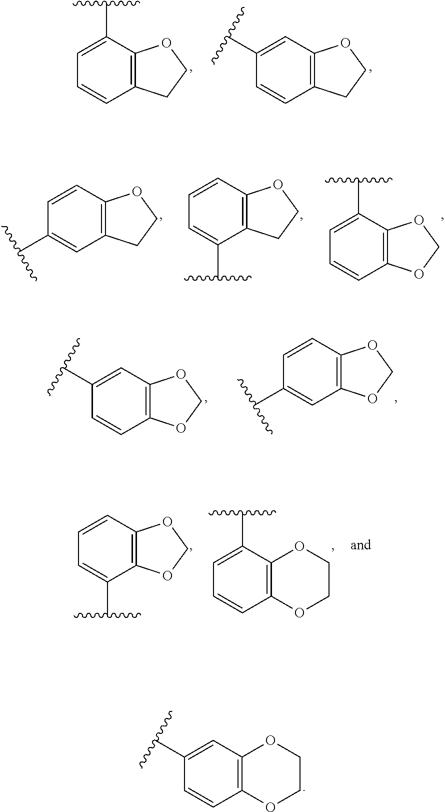

In some embodiments, compounds of Formula (IIa) include, but are not limited to, those of Formula (IIf):

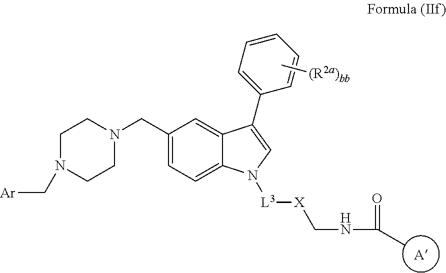

For compounds of Formula (IIf), L³-X is a substituted C₃alkylene. Each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, R^{2a} is —CH₂NH₂. In some embodiments, bb is 0. In some embodiments bb is 1. In some embodiments, R^{2a} is halogen and bb is 1. In some embodiments, R^{2a} is —OCF₃ and bb is 1. In some embodiments, Ar is unsubstituted phenyl. In some embodiments, Ar is a substituted phenyl. In some embodiments Ar is selected from:

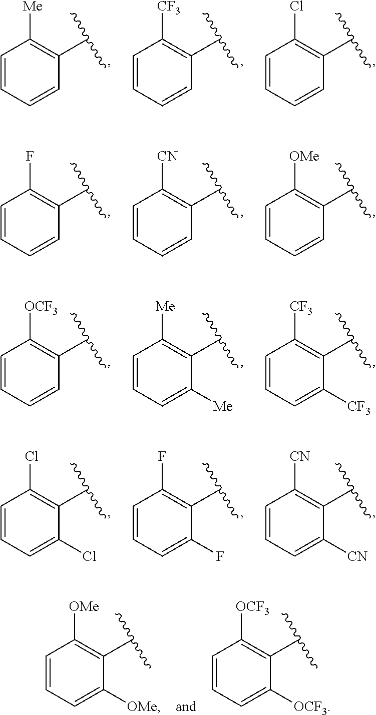

In some embodiments,

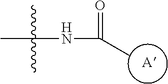

is selected from

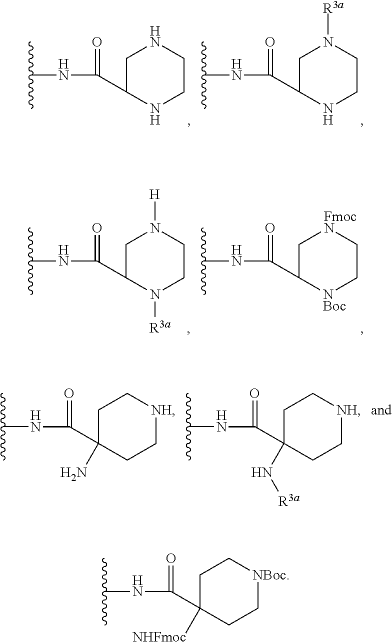

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

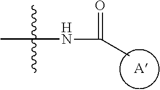 [SMILES: *NC(C)=O]

is selected from

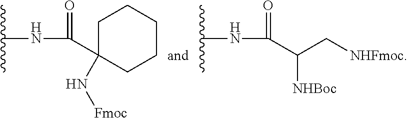

In some embodiments, compounds of Formula (IIa) include, but are not limited to, those of Formula (IIg):

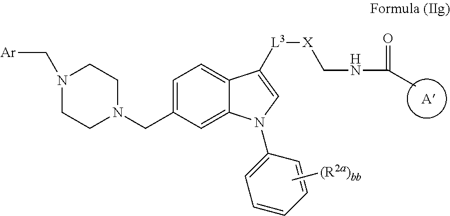

For compounds of Formula (IIg), L³-X is a substituted C₃alkylene. Each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, R^{2a} is —CH₂NH₂. In some embodiments, bb is 0. In some embodiments bb is 1. In some embodiments, R^{2a} is halogen and bb is 1. In some embodiments, R^{2a} is —OCF₃ and bb is 1. In some embodiments, Ar is unsubstituted phenyl. In some embodiments, Ar is a substituted phenyl. In some embodiments Ar is selected from:

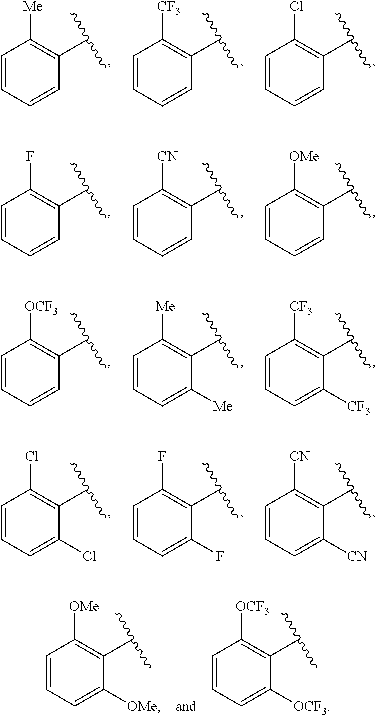

In some embodiments,

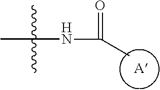 [SMILES: *NC(C)=O]

is selected from

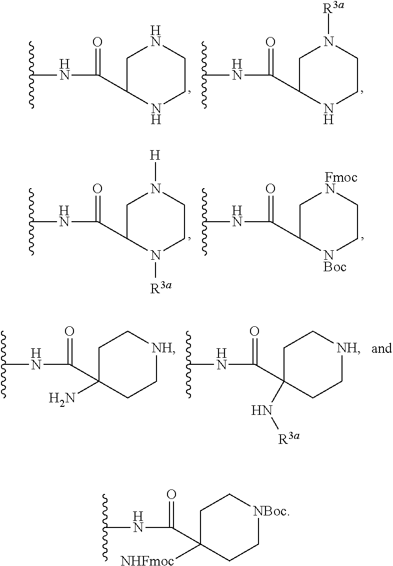

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

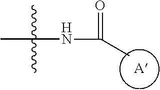 [SMILES: *NC(C)=O]

is selected from

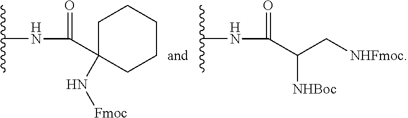

Also provided herein is a compound having a formula selected from:

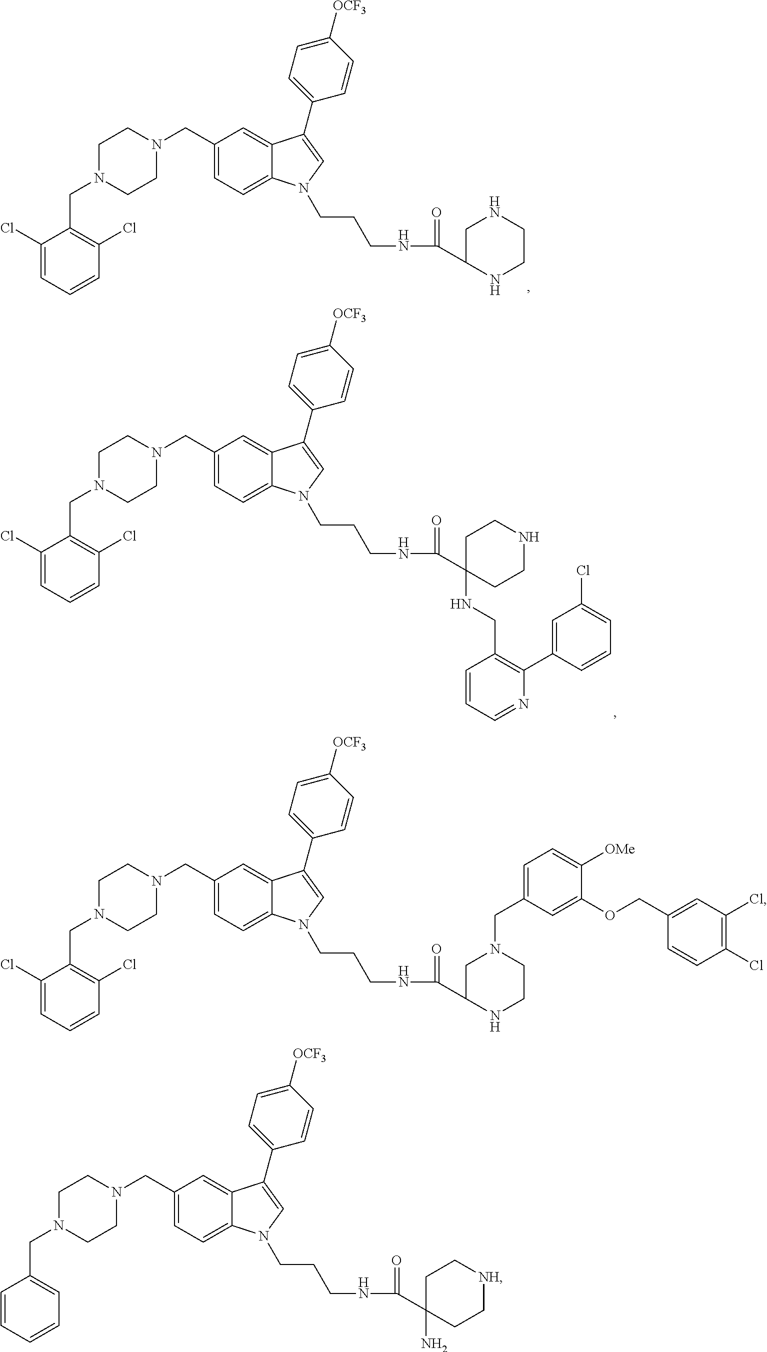 [SMILES: O=C(NCCCn1cc(-c2ccc(OC(F)(F)F)cc2)c2cc(CN3CCN(Cc4c(Cl)cccc4Cl)CC3)ccc21)C1CNCCN1 ; O=C(NCCCn1cc(-c2ccc(OC(F)(F)F)cc2)c2cc(CN3CCN(Cc4c(Cl)cccc4Cl)CC3)ccc21)C1(NCc2cccnc2-c2cccc(Cl)c2)CCNCC1 ; COc1ccc(CN2CCNC(C(=O)NCCCn3cc(-c4ccc(OC(F)(F)F)cc4)c4cc(CN5CCN(Cc6c(Cl)cccc6Cl)CC5)ccc43)C2)cc1OCc1ccc(Cl)c(Cl)c1 ; NC1(C(=O)NCCCn2cc(-c3ccc(OC(F)(F)F)cc3)c3cc(CN4CCN(Cc5ccccc5)CC4)ccc32)CCNCC1]

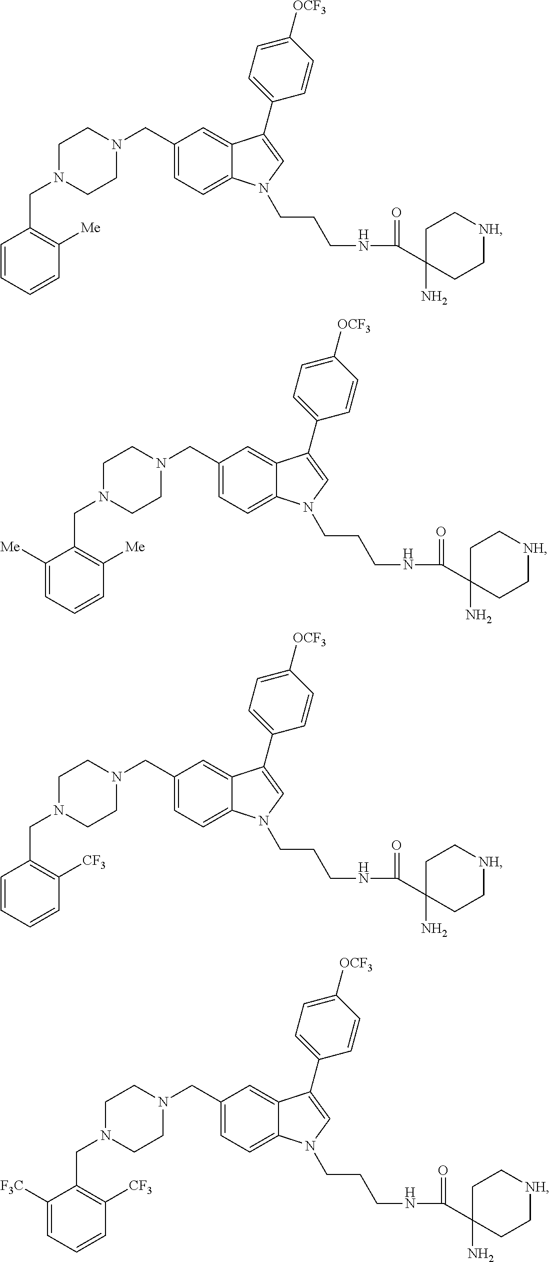 [SMILES: Cc1ccccc1CN1CCN(Cc2ccc3c(c2)c(-c2ccc(OC(F)(F)F)cc2)cn3CCCNC(=O)C2(N)CCNCC2)CC1 ; Cc1cccc(C)c1CN1CCN(Cc2ccc3c(c2)c(-c2ccc(OC(F)(F)F)cc2)cn3CCCNC(=O)C2(N)CCNCC2)CC1 ; NC1(C(=O)NCCCn2cc(-c3ccc(OC(F)(F)F)cc3)c3cc(CN4CCN(Cc5ccccc5C(F)(F)F)CC4)ccc32)CCNCC1 ; NC1(C(=O)NCCCn2cc(-c3ccc(OC(F)(F)F)cc3)c3cc(CN4CCN(Cc5c(C(F)(F)F)cccc5C(F)(F)F)CC4)ccc32)CCNCC1]

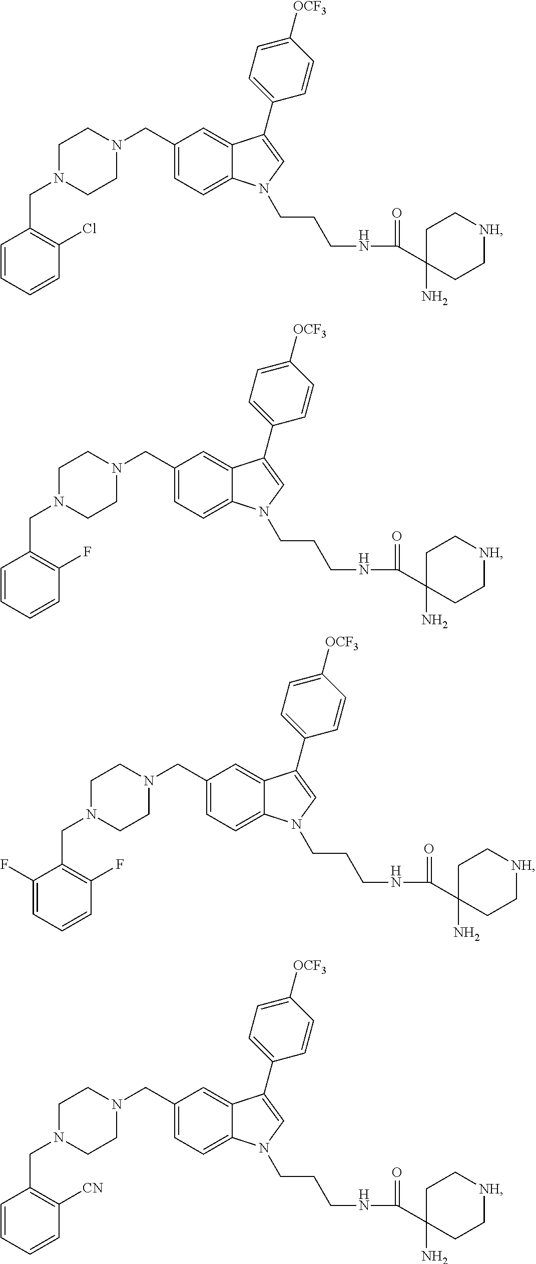 [SMILES: NC1(C(=O)NCCCn2cc(-c3ccc(OC(F)(F)F)cc3)c3cc(CN4CCN(Cc5ccccc5Cl)CC4)ccc32)CCNCC1 ; NC1(C(=O)NCCCn2cc(-c3ccc(OC(F)(F)F)cc3)c3cc(CN4CCN(Cc5ccccc5F)CC4)ccc32)CCNCC1 ; NC1(C(=O)NCCCn2cc(-c3ccc(OC(F)(F)F)cc3)c3cc(CN4CCN(Cc5c(F)cccc5F)CC4)ccc32)CCNCC1 ; N#Cc1ccccc1CN1CCN(Cc2ccc3c(c2)c(-c2ccc(OC(F)(F)F)cc2)cn3CCCNC(=O)C2(N)CCNCC2)CC1]

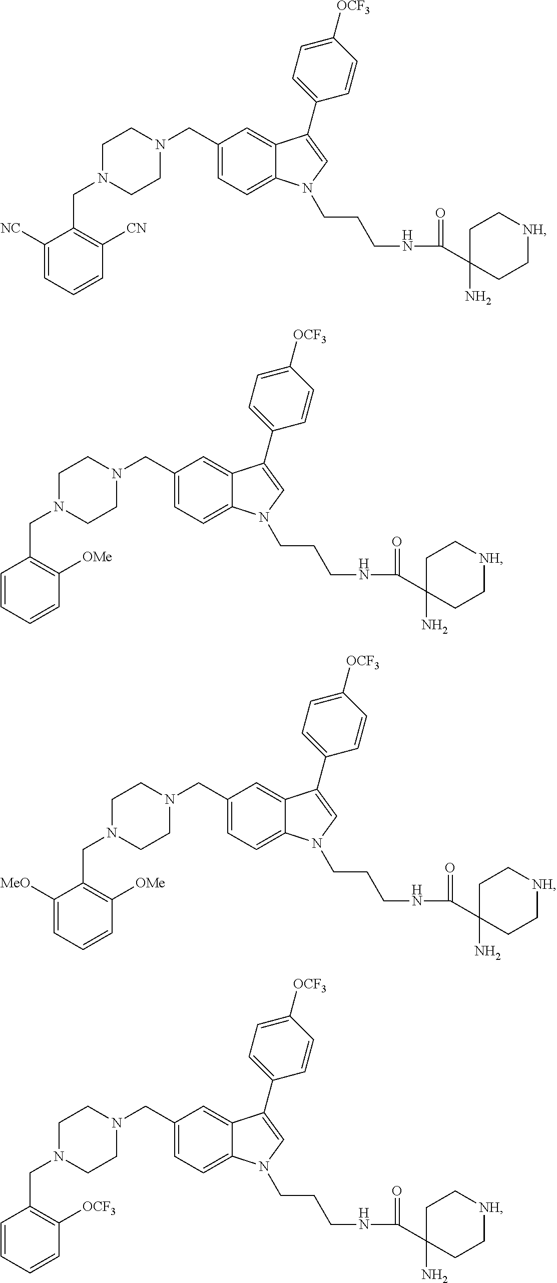 [SMILES: N#Cc1cccc(C#N)c1CN1CCN(Cc2ccc3c(c2)c(-c2ccc(OC(F)(F)F)cc2)cn3CCCNC(=O)C2(N)CCNCC2)CC1 ; COc1ccccc1CN1CCN(Cc2ccc3c(c2)c(-c2ccc(OC(F)(F)F)cc2)cn3CCCNC(=O)C2(N)CCNCC2)CC1 ; COc1cccc(OC)c1CN1CCN(Cc2ccc3c(c2)c(-c2ccc(OC(F)(F)F)cc2)cn3CCCNC(=O)C2(N)CCNCC2)CC1 ; NC1(C(=O)NCCCn2cc(-c3ccc(OC(F)(F)F)cc3)c3cc(CN4CCN(Cc5ccccc5OC(F)(F)F)CC4)ccc32)CCNCC1]

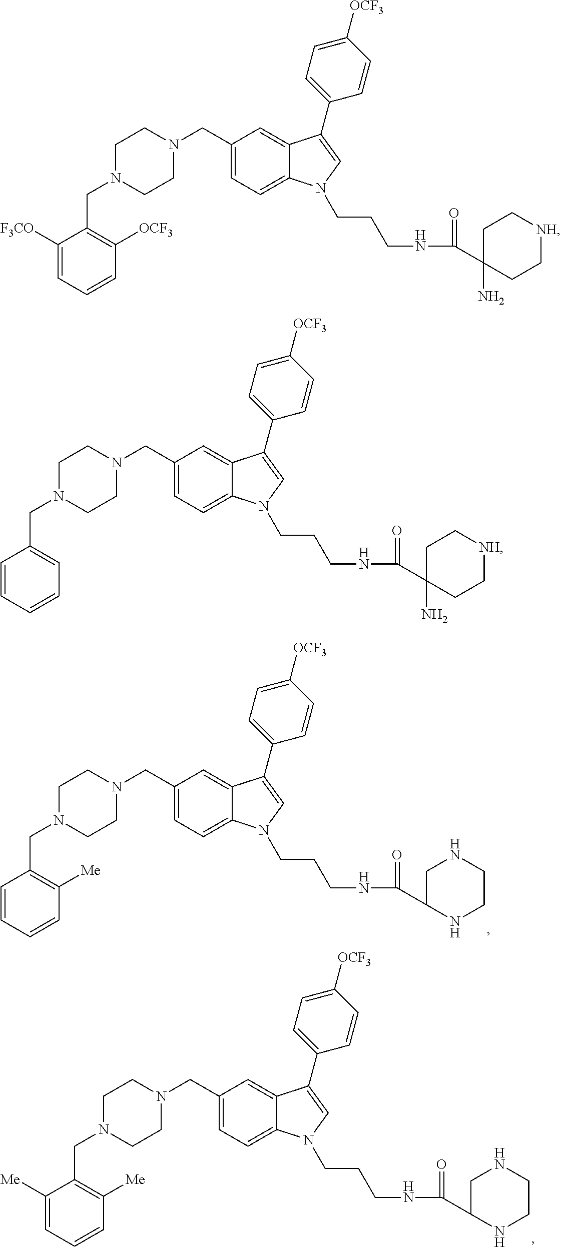 [SMILES: NC1(C(=O)NCCCn2cc(-c3ccc(OC(F)(F)F)cc3)c3cc(CN4CCN(Cc5c(OC(F)(F)F)cccc5OC(F)(F)F)CC4)ccc32)CCNCC1 ; NC1(C(=O)NCCCn2cc(-c3ccc(OC(F)(F)F)cc3)c3cc(CN4CCN(Cc5ccccc5)CC4)ccc32)CCNCC1 ; Cc1ccccc1CN1CCN(Cc2ccc3c(c2)c(-c2ccc(OC(F)(F)F)cc2)cn3CCCNC(=O)C2CNCCN2)CC1 ; Cc1cccc(C)c1CN1CCN(Cc2ccc3c(c2)c(-c2ccc(OC(F)(F)F)cc2)cn3CCCNC(=O)C2CNCCN2)CC1]

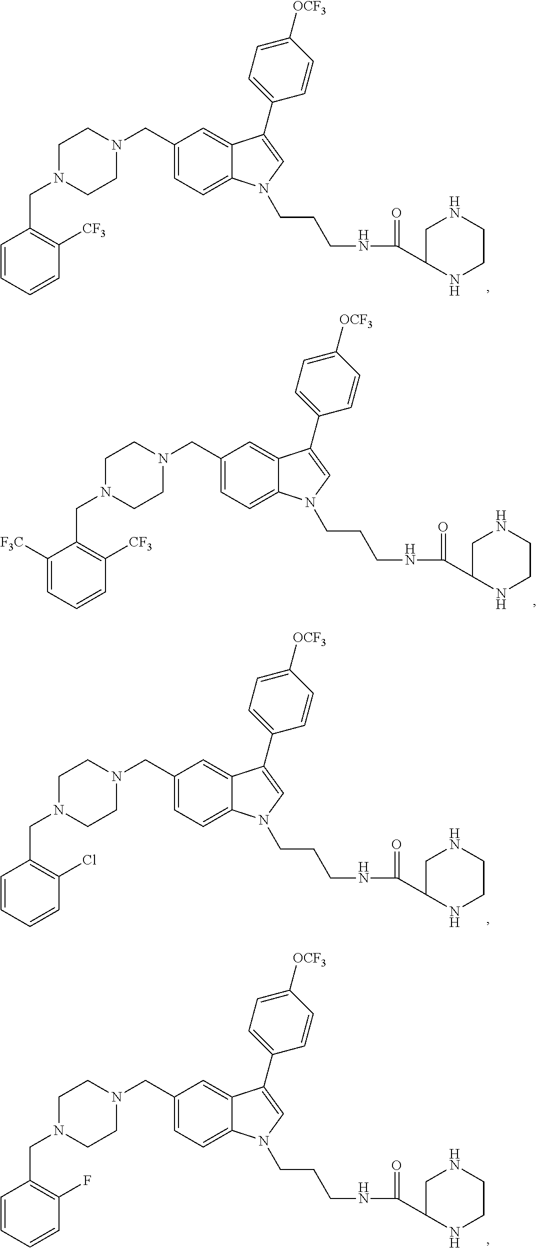 [SMILES: O=C(NCCCn1cc(-c2ccc(OC(F)(F)F)cc2)c2cc(CN3CCN(Cc4ccccc4C(F)(F)F)CC3)ccc21)C1CNCCN1 ; O=C(NCCCn1cc(-c2ccc(OC(F)(F)F)cc2)c2cc(CN3CCN(Cc4c(C(F)(F)F)cccc4C(F)(F)F)CC3)ccc21)C1CNCCN1 ; O=C(NCCCn1cc(-c2ccc(OC(F)(F)F)cc2)c2cc(CN3CCN(Cc4ccccc4Cl)CC3)ccc21)C1CNCCN1 ; O=C(NCCCn1cc(-c2ccc(OC(F)(F)F)cc2)c2cc(CN3CCN(Cc4ccccc4F)CC3)ccc21)C1CNCCN1]

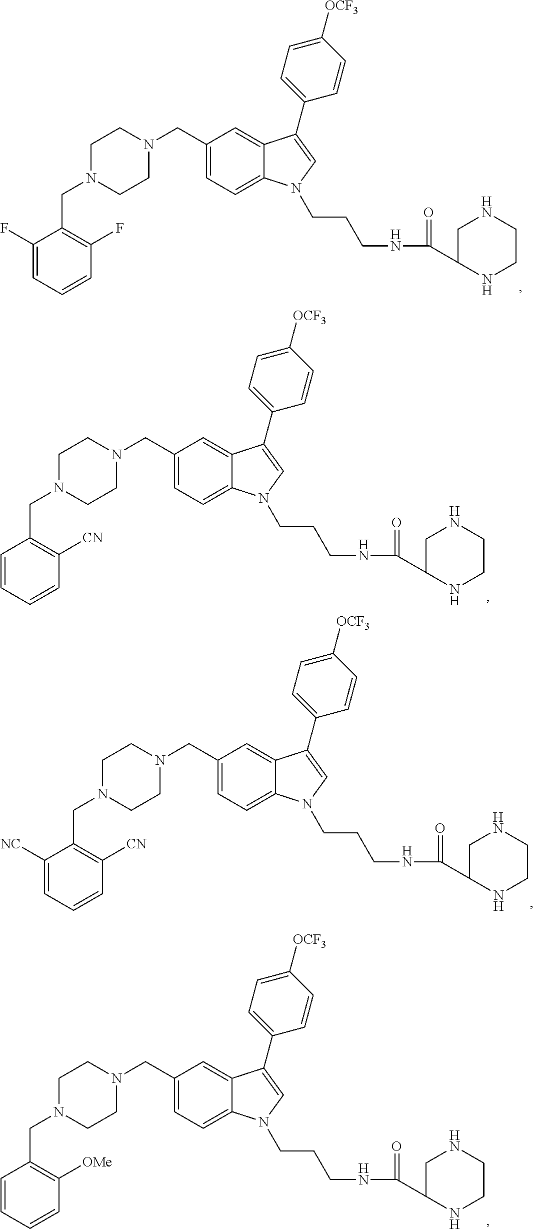 [SMILES: O=C(NCCCn1cc(-c2ccc(OC(F)(F)F)cc2)c2cc(CN3CCN(Cc4c(F)cccc4F)CC3)ccc21)C1CNCCN1 ; N#Cc1ccccc1CN1CCN(Cc2ccc3c(c2)c(-c2ccc(OC(F)(F)F)cc2)cn3CCCNC(=O)C2CNCCN2)CC1 ; N#Cc1cccc(C#N)c1CN1CCN(Cc2ccc3c(c2)c(-c2ccc(OC(F)(F)F)cc2)cn3CCCNC(=O)C2CNCCN2)CC1 ; COc1ccccc1CN1CCN(Cc2ccc3c(c2)c(-c2ccc(OC(F)(F)F)cc2)cn3CCCNC(=O)C2CNCCN2)CC1]

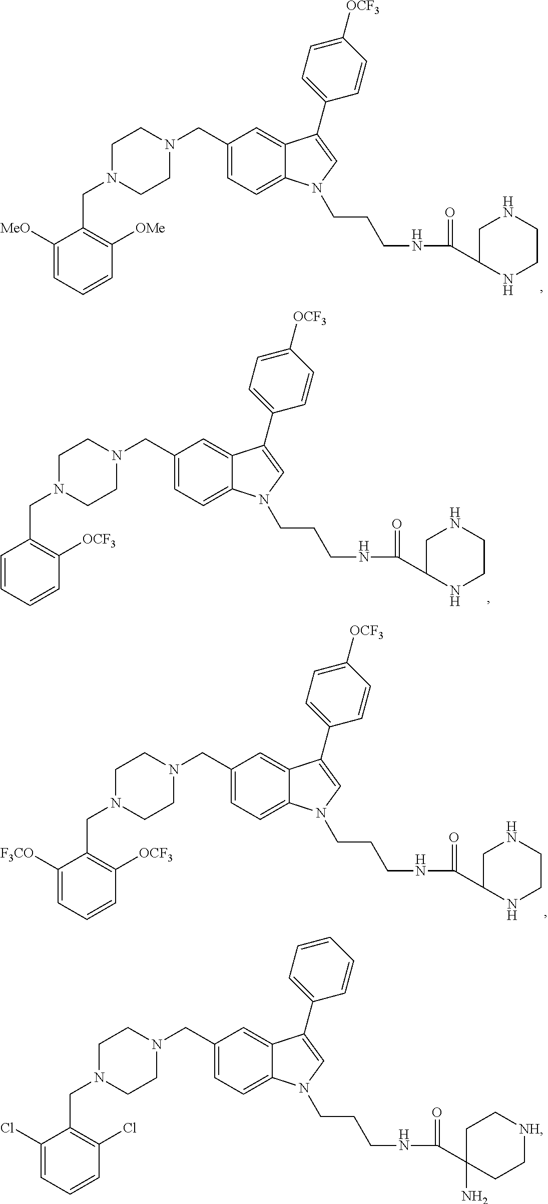 [SMILES: COc1cccc(OC)c1CN1CCN(Cc2ccc3c(c2)c(-c2ccc(OC(F)(F)F)cc2)cn3CCCNC(=O)C2CNCCN2)CC1 ; O=C(NCCCn1cc(-c2ccc(OC(F)(F)F)cc2)c2cc(CN3CCN(Cc4ccccc4OC(F)(F)F)CC3)ccc21)C1CNCCN1 ; O=C(NCCCn1cc(-c2ccc(OC(F)(F)F)cc2)c2cc(CN3CCN(Cc4c(OC(F)(F)F)cccc4OC(F)(F)F)CC3)ccc21)C1CNCCN1 ; NC1(C(=O)NCCCn2cc(-c3ccccc3)c3cc(CN4CCN(Cc5c(Cl)cccc5Cl)CC4)ccc32)CCNCC1]

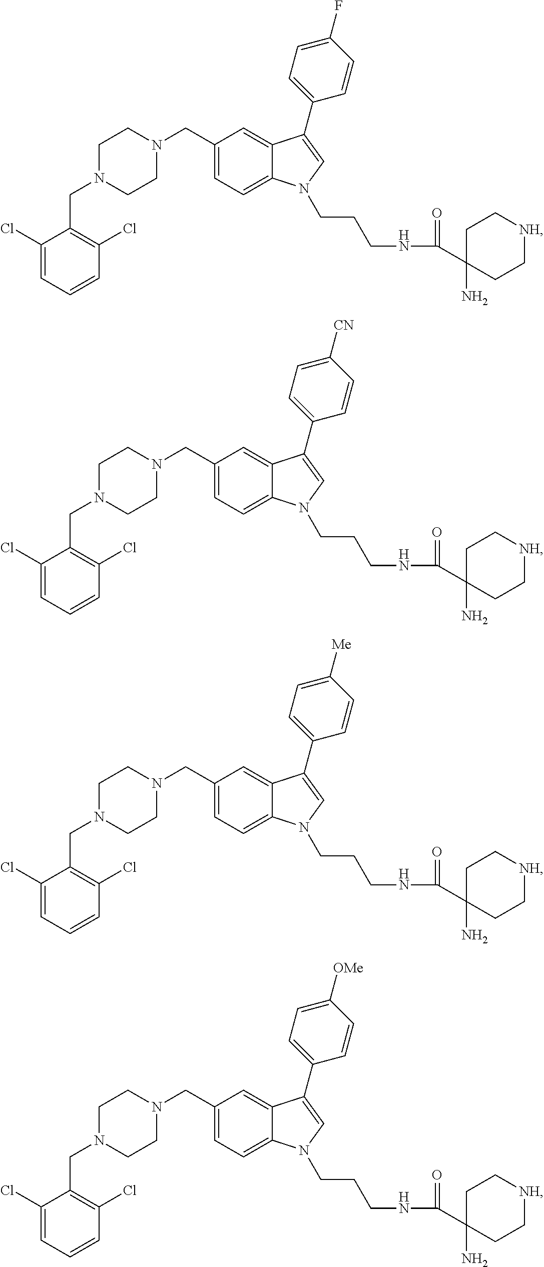 [SMILES: NC1(C(=O)NCCCn2cc(-c3ccc(F)cc3)c3cc(CN4CCN(Cc5c(Cl)cccc5Cl)CC4)ccc32)CCNCC1 ; N#Cc1ccc(-c2cn(CCCNC(=O)C3(N)CCNCC3)c3ccc(CN4CCN(Cc5c(Cl)cccc5Cl)CC4)cc23)cc1 ; Cc1ccc(-c2cn(CCCNC(=O)C3(N)CCNCC3)c3ccc(CN4CCN(Cc5c(Cl)cccc5Cl)CC4)cc23)cc1 ; COc1ccc(-c2cn(CCCNC(=O)C3(N)CCNCC3)c3ccc(CN4CCN(Cc5c(Cl)cccc5Cl)CC4)cc23)cc1]

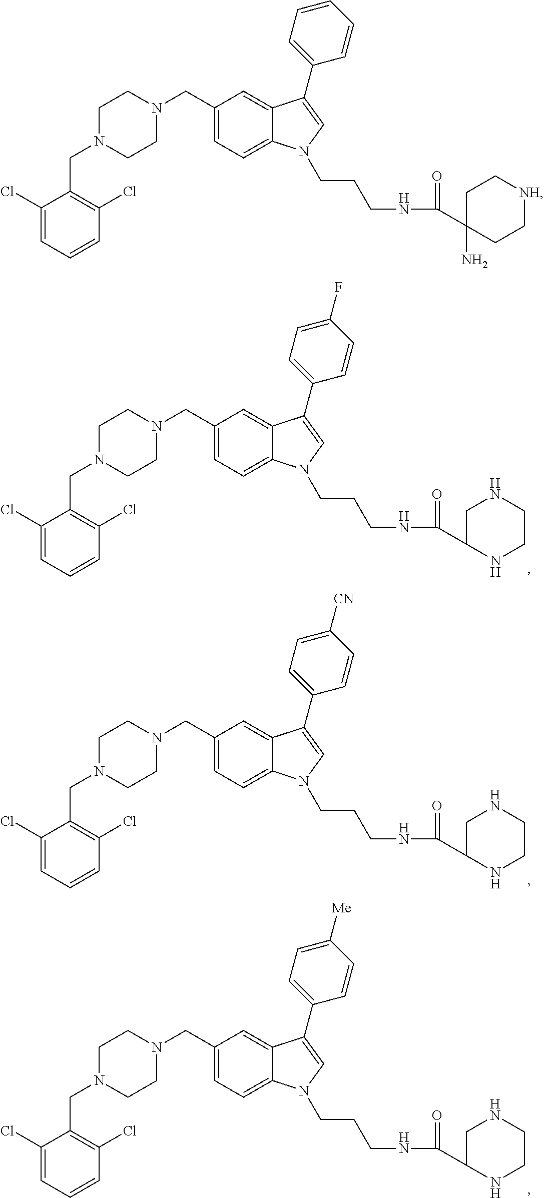 [SMILES: NC1(C(=O)NCCCn2cc(-c3ccccc3)c3cc(CN4CCN(Cc5c(Cl)cccc5Cl)CC4)ccc32)CCNCC1 ; O=C(NCCCn1cc(-c2ccc(F)cc2)c2cc(CN3CCN(Cc4c(Cl)cccc4Cl)CC3)ccc21)C1CNCCN1 ; N#Cc1ccc(-c2cn(CCCNC(=O)C3CNCCN3)c3ccc(CN4CCN(Cc5c(Cl)cccc5Cl)CC4)cc23)cc1 ; Cc1ccc(-c2cn(CCCNC(=O)C3CNCCN3)c3ccc(CN4CCN(Cc5c(Cl)cccc5Cl)CC4)cc23)cc1]

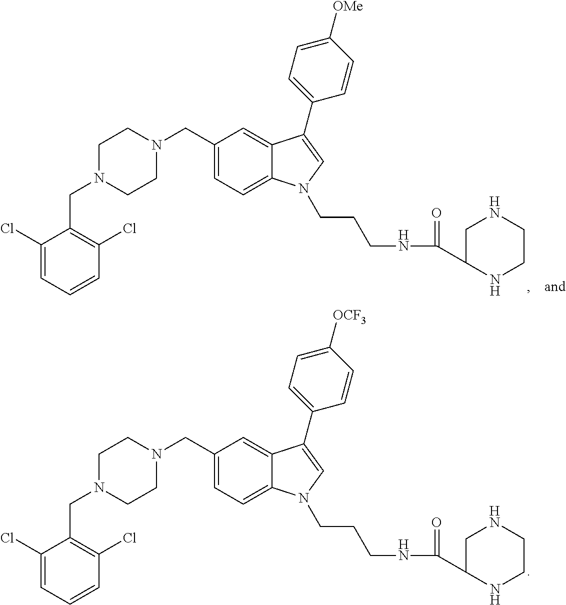 [SMILES: COc1ccc(-c2cn(CCCNC(=O)C3CNCCN3)c3ccc(CN4CCN(Cc5c(Cl)cccc5Cl)CC4)cc23)cc1 ; O=C(NCCCn1cc(-c2ccc(OC(F)(F)F)cc2)c2cc(CN3CCN(Cc4c(Cl)cccc4Cl)CC3)ccc21)C1CNCCN1]

In some embodiments, the compound is selected from any one of the compounds from the following table:

TABLE 5

| Compound | | |
|---|---|---|
| Ref. | Compound | Chemical Name |
| C24 | 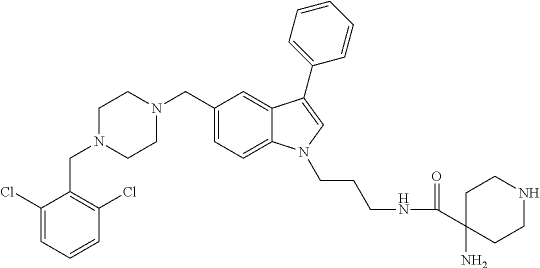 [SMILES: NC1(C(=O)NCCCn2cc(-c3ccccc3)c3cc(CN4CCN(Cc5c(Cl)cccc5Cl)CC4)ccc32)CCNCC1] | 4-amino-N-(3-(5-((4-(2,6- dichlorobenzyl)piperazin-1- yl)methyl)-3-phenyl-1H- indol-1-yl)propyl)piperidine- 4-carboxamide |
| C39 | 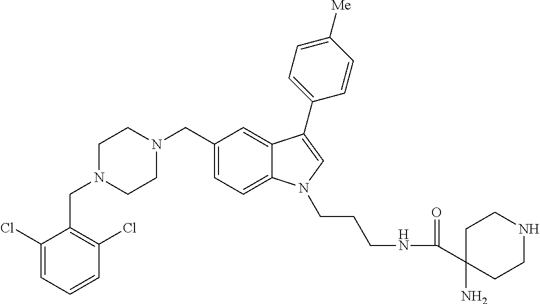 [SMILES: Cc1ccc(-c2cn(CCCNC(=O)C3(N)CCNCC3)c3ccc(CN4CCN(Cc5c(Cl)cccc5Cl)CC4)cc23)cc1] | 4-amino-N-(3-(5-((4-(2,6- dichlorobenzyl)piperazin-1- yl)methyl)-3-p-tolyl-1H- indol-1-yl)propyl)piperidine- 4-carboxamide |
| C40 | 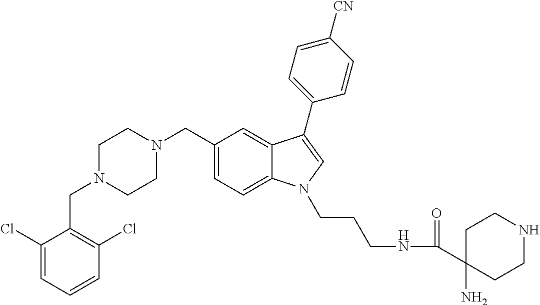 [SMILES: N#Cc1ccc(-c2cn(CCCNC(=O)C3(N)CCNCC3)c3ccc(CN4CCN(Cc5c(Cl)cccc5Cl)CC4)cc23)cc1] | 4-amino-N-(3-(3-(4- cyanophenyl)-5-((4-(2,6- dichlorobenzyl)piperazin-1- yl)methyl)-1H-indol-1- yl)propyl)piperidine-4- carboxamide |
| C49 | 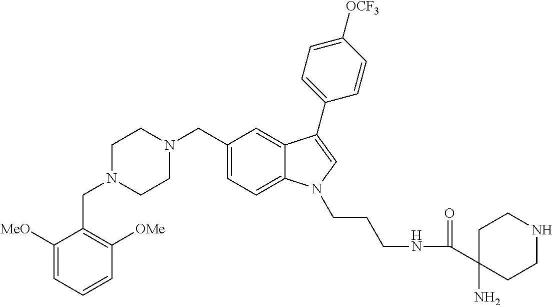 [SMILES: COc1cccc(OC)c1CN1CCN(Cc2ccc3c(c2)c(-c2ccc(OC(F)(F)F)cc2)cn3CCCNC(=O)C2(N)CCNCC2)CC1] | 4-amino-N-(3-(5-((4-(2,6- dimethoxybenzyl)piperazin-1- yl)methyl)-3-(4- (trifluoromethoxy)phenyl)- 1H-indol-1- yl)propyl)piperidine-4- carboxamide |
| C54 | 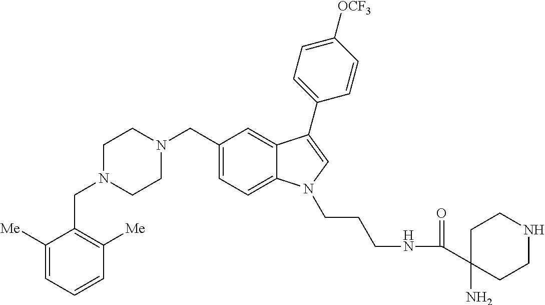 [SMILES: Cc1cccc(C)c1CN1CCN(Cc2ccc3c(c2)c(-c2ccc(OC(F)(F)F)cc2)cn3CCCNC(=O)C2(N)CCNCC2)CC1] | 4-amino-N-(3-(5-((4-(2,6- dimethylbenzyl)piperazin-1- yl)methyl)-3-(4- (trifluoromethoxy)phenyl)- 1H-indol-1- yl)propyl)piperidine-4- carboxamide |
| C55 | 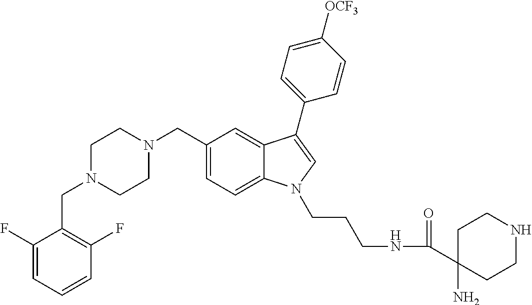 [SMILES: NC1(C(=O)NCCCn2cc(-c3ccc(OC(F)(F)F)cc3)c3cc(CN4CCN(Cc5c(F)cccc5F)CC4)ccc32)CCNCC1] | 4-amino-N-(3-(5-((4-(2,6- difluorobenzyl)piperazin-1- yl)methyl)-3-(4- (trifluoromethoxy)phenyl)- 1H-indol-1- yl)propyl)piperidine-4- carboxamide |
| C58 | 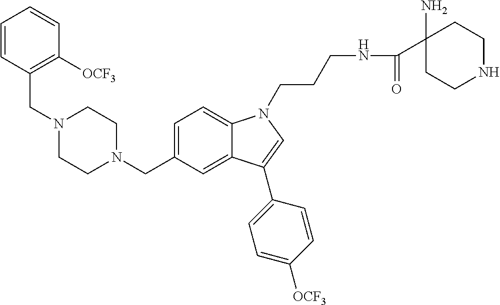 [SMILES: NC1(C(=O)NCCCn2cc(-c3ccc(OC(F)(F)F)cc3)c3cc(CN4CCN(Cc5ccccc5OC(F)(F)F)CC4)ccc32)CCNCC1] | 4-amino-N-(3-(5-((4-(2- (trifluoromethoxy)benzyl) piperazin-1-yl)methyl)-3-(4- (trifluoromethoxy)phenyl)- 1H-indol-1- yl)propyl)piperidine-4- carboxamide |
| C59 | 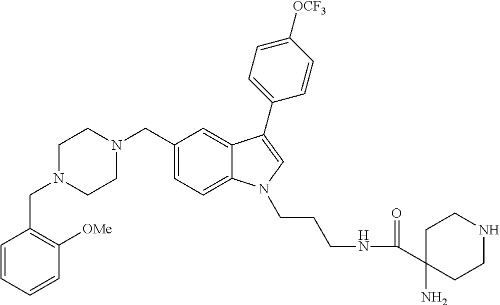 [SMILES: COc1ccccc1CN1CCN(Cc2ccc3c(c2)c(-c2ccc(OC(F)(F)F)cc2)cn3CCCNC(=O)C2(N)CCNCC2)CC1] | 4-amino-N-(3-(5-((4-(2- methoxybenzyl)piperazin-1- yl)methyl)-3-(4- (trifluoromethoxy)phenyl)- 1H-indol-1- yl)propyl)piperidine-4- carboxamide |
| C60 | 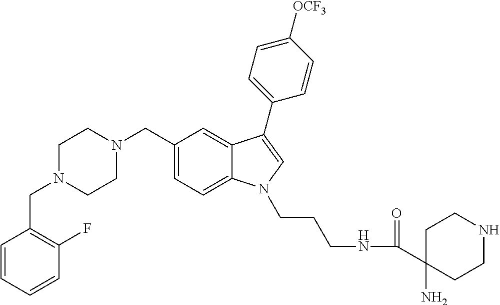 [SMILES: NC1(C(=O)NCCCn2cc(-c3ccc(OC(F)(F)F)cc3)c3cc(CN4CCN(Cc5ccccc5F)CC4)ccc32)CCNCC1] | 4-amino-N-(3-(5-((4-(2- fluorobenzyl)piperazin-1- yl)methyl)-3-(4- (trifluoromethoxy)phenyl)- 1H-indol-1- yl)propyl)piperidine-4- carboxamide |
| C62 | 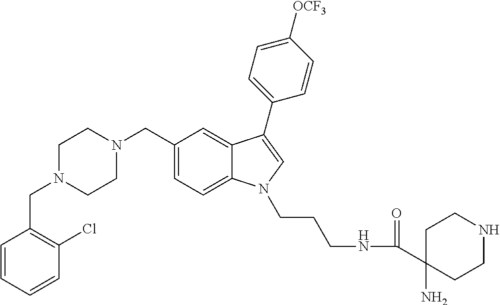 [SMILES: NC1(C(=O)NCCCn2cc(-c3ccc(OC(F)(F)F)cc3)c3cc(CN4CCN(Cc5ccccc5Cl)CC4)ccc32)CCNCC1] | 4-amino-N-(3-(5-((4-(2- chlorobenzyl)piperazin-1- yl)methyl)-3-(4- (trifluoromethoxy)phenyl)- 1H-indol-1- yl)propyl)piperidine-4- carboxamide |
| C63 | 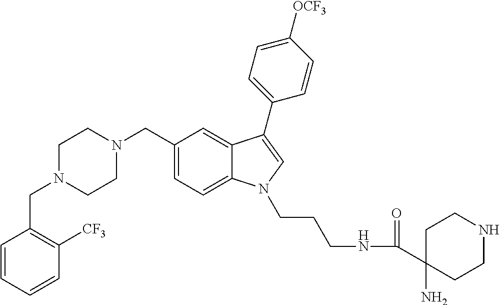 [SMILES: NC1(C(=O)NCCCn2cc(-c3ccc(OC(F)(F)F)cc3)c3cc(CN4CCN(Cc5ccccc5C(F)(F)F)CC4)ccc32)CCNCC1] | 4-amino-N-(3-(3-(4- (trifluoromethoxy)phenyl)-5- ((4-(2-(trifluoromethyl)benzyl) piperazin-1-yl)methyl)-1H- indol-1-yl)propyl)piperidine-4- carboxamide |
| C66 | 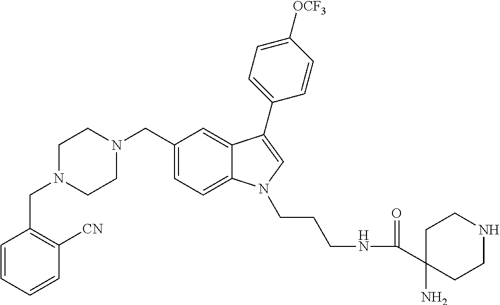 [SMILES: N#Cc1ccccc1CN1CCN(Cc2ccc3c(c2)c(-c2ccc(OC(F)(F)F)cc2)cn3CCCNC(=O)C2(N)CCNCC2)CC1] | 4-amino-N-(3-(5-((4-(2- cyanobenzyl)piperazin-1- yl)methyl)-3-(4- (trifluoromethoxy)phenyl)- 1H-indol-1- yl)propyl)piperidine-4- carboxamide |
| C71 | 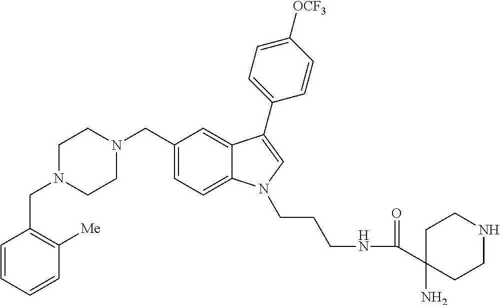 [SMILES: Cc1ccccc1CN1CCN(Cc2ccc3c(c2)c(-c2ccc(OC(F)(F)F)cc2)cn3CCCNC(=O)C2(N)CCNCC2)CC1] | 4-amino-N-(3-(5-((4-(2- methylbenzyl)piperazin-1- yl)methyl)-3-(4- (trifluoromethoxy)phenyl)- 1H-indol-1- yl)propyl)piperidine-4- carboxamide |
| C72 | 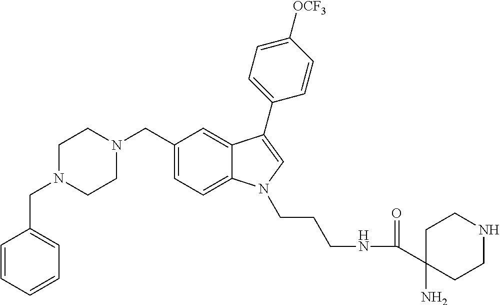 [SMILES: NC1(C(=O)NCCCn2cc(-c3ccc(OC(F)(F)F)cc3)c3cc(CN4CCN(Cc5ccccc5)CC4)ccc32)CCNCC1] | 4-amino-N-(3-(5-((4- benzylpiperazin-1-yl)methyl)- 3-(4- (trifluoromethoxy)phenyl)- 1H-indol-1- yl)propyl)piperidine-4- carboxamide |
| C89 | 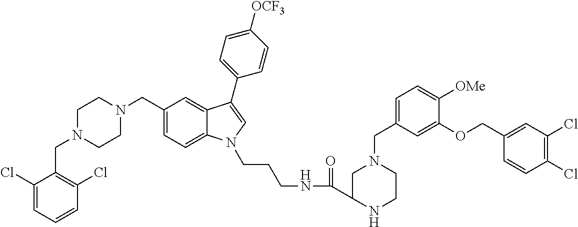 [SMILES: COc1ccc(CN2CCNC(C(=O)NCCCn3cc(-c4ccc(OC(F)(F)F)cc4)c4cc(CN5CCN(Cc6c(Cl)cccc6Cl)CC5)ccc43)C2)cc1OCc1ccc(Cl)c(Cl)c1] | N-(3-(5-((4-(2,6- dichlorobenzyl)piperazin-1- yl)methyl)-3-(4- (trifluoromethoxy)phenyl)- 1H-indol-1-yl)propyl)-4-(3- (3,4-dichlorobenzyloxy)-4- methoxybenzyl)piperazine-2- carboxamide |
| C90 | 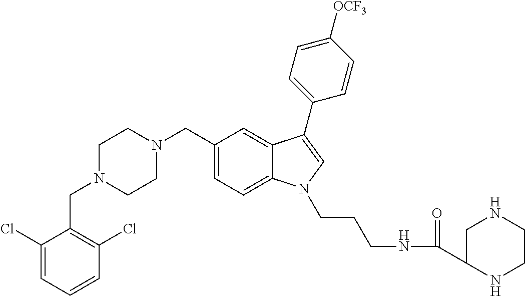 [SMILES: O=C(NCCCn1cc(-c2ccc(OC(F)(F)F)cc2)c2cc(CN3CCN(Cc4c(Cl)cccc4Cl)CC3)ccc21)C1CNCCN1] | N-(3-(5-((4-(2,6- dichlorobenzyl)piperazin-1- yl)methyl)-3-(4- (trifluoromethoxy)phenyl)- 1H-indol-1- yl)propyl)piperazine-2- carboxamide |
| C114 | 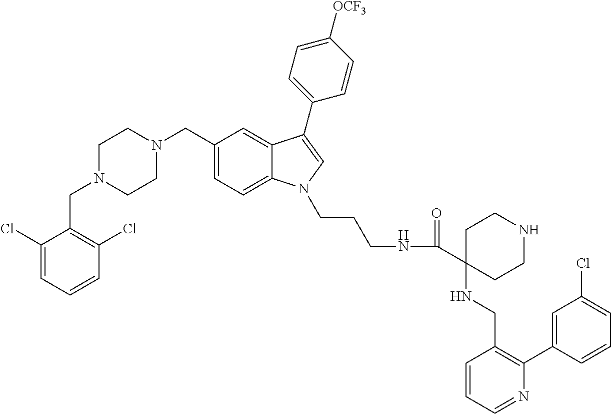 [SMILES: O=C(NCCCn1cc(-c2ccc(OC(F)(F)F)cc2)c2cc(CN3CCN(Cc4c(Cl)cccc4Cl)CC3)ccc21)C1(NCc2cccnc2-c2cccc(Cl)c2)CCNCC1] | 4-((2-(3- chlorophenyl)pyridin-3- yl)methylamino)-N-(3-(5-((4- (2,6- dichlorobenzyl)piperazin-1- yl)methyl)-3-(4- (trifluoromethoxy)phenyl)- 1H-indol-1- yl)propyl)piperidine-4- carboxamide |

In some embodiments, the compound is selected from any one of the compounds from the following table:

TABLE 6A

| Com- | | |
|---|---|---|
| pound | | |
| Ref. | Compound | Chemical Name |
| C16 | 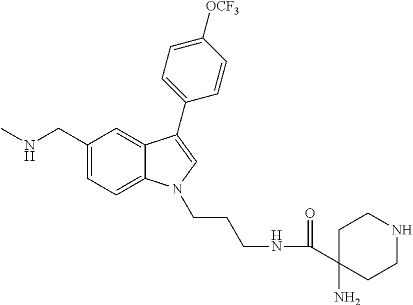 [SMILES: CNCc1ccc2c(c1)c(-c1ccc(OC(F)(F)F)cc1)cn2CCCNC(=O)C1(N)CCNCC1] | 4-amino-N-(3-(5- ((methylamino)methyl)-3-(4- (trifluoromethoxy)phenyl)- 1H-indol-1- yl)propyl)piperidine-4- carboxamide |
| C25 | 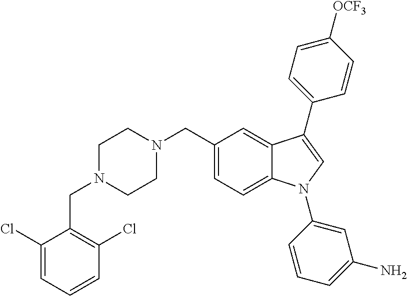 [SMILES: Nc1cccc(-n2cc(-c3ccc(OC(F)(F)F)cc3)c3cc(CN4CCN(Cc5c(Cl)cccc5Cl)CC4)ccc32)c1] | 3-(5-((4-(2,6- dichlorobenzyl)piperazin-1- yl)methyl)-3-(4- (trifluoromethoxy)phenyl)- 1H-indol-1-yl)aniline |
| C29 | 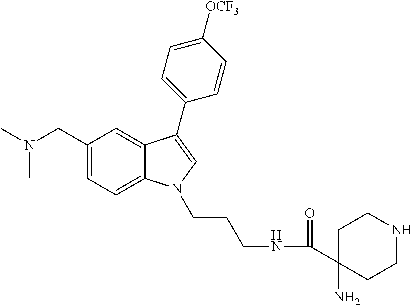 [SMILES: CN(C)Cc1ccc2c(c1)c(-c1ccc(OC(F)(F)F)cc1)cn2CCCNC(=O)C1(N)CCNCC1] | 4-amino-N-(3-(5- ((dimethylamino)methyl)-3- (4-(trifluoromethoxy)phenyl)- 1H-indol-1- yl)propyl)piperidine-4- carboxamide |
| C35 | 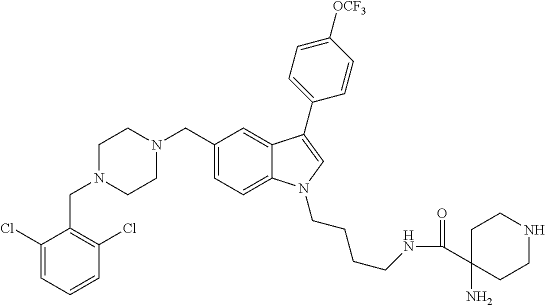 [SMILES: NC1(C(=O)NCCCCn2cc(-c3ccc(OC(F)(F)F)cc3)c3cc(CN4CCN(Cc5c(Cl)cccc5Cl)CC4)ccc32)CCNCC1] | 4-amino-N-(4-(5-((4-(2,6- dichlorobenzyl)piperazin-1- yl)methyl)-3-(4- (trifluoromethoxy)phenyl)- 1H-indol-1- yl)butyl)piperidine-4- carboxamide |
| C36 | 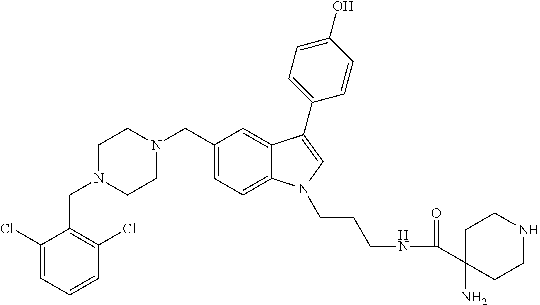 [SMILES: NC1(C(=O)NCCCn2cc(-c3ccc(O)cc3)c3cc(CN4CCN(Cc5c(Cl)cccc5Cl)CC4)ccc32)CCNCC1] | 4-amino-N-(3-(5-((4-(2,6- dichlorobenzyl)piperazin-1- yl)methyl)-3-(4- hydroxyphenyl)-1H-indol-1- yl)propyl)piperidine-4- carboxamide |
| C41 | 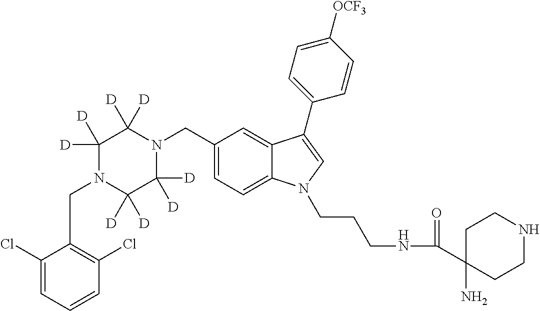 [SMILES: [2H]C1([2H])N(Cc2ccc3c(c2)c(-c2ccc(OC(F)(F)F)cc2)cn3CCCNC(=O)C2(N)CCNCC2)C([2H])([2H])C([2H])([2H])N(Cc2c(Cl)cccc2Cl)C1([2H])[2H]] | |
| C64 | 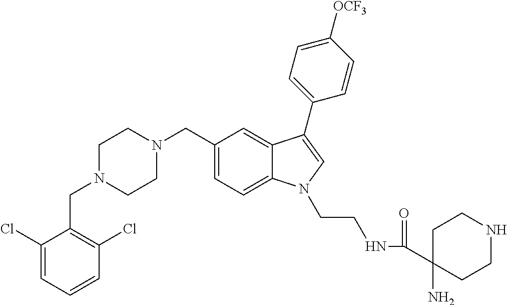 [SMILES: NC1(C(=O)NCCn2cc(-c3ccc(OC(F)(F)F)cc3)c3cc(CN4CCN(Cc5c(Cl)cccc5Cl)CC4)ccc32)CCNCC1] | 4-amino-N-(2-(5-((4-(2,6- dichlorobenzyl)piperazin-1- yl)methyl)-3-(4- (trifluoromethoxy)phenyl)- 1H-indol-1- yl)ethyl)piperidine-4- carboxamide |
| C82 | 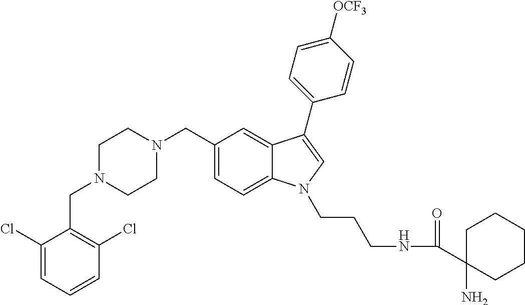 [SMILES: NC1(C(=O)NCCCn2cc(-c3ccc(OC(F)(F)F)cc3)c3cc(CN4CCN(Cc5c(Cl)cccc5Cl)CC4)ccc32)CCCCC1] | 1-amino-N-(3-(5-((4-(2,6- dichlorobenzyl)piperazin-1- yl)methyl)-3-(4- (trifluoromethoxy)phenyl)- 1H-indol-1-yl)propyl) cyclohexanecarboxamide |
| C91 | 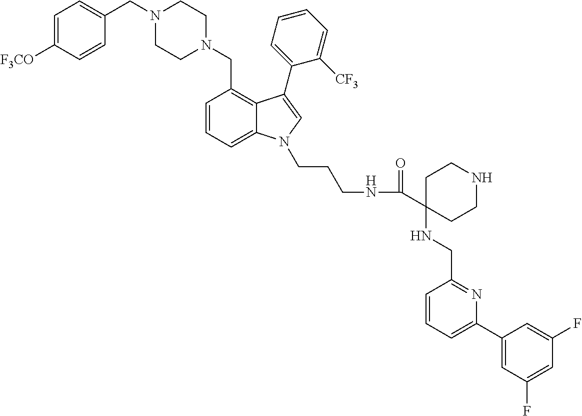 [SMILES: O=C(NCCCn1cc(-c2ccccc2C(F)(F)F)c2c(CN3CCN(Cc4ccc(OC(F)(F)F)cc4)CC3)cccc21)C1(NCc2cccc(-c3cc(F)cc(F)c3)n2)CCNCC1] | 4-((6-(3,5- difluorophenyl)pyridin-2- yl)methylamino)-N-(3-(4-((4- (4-(trifluoromethoxy)benzyl) piperazin-1-yl)methyl)-3-(2- (trifluoromethyl)phenyl)-1H- indol-1-yl)propyl)piperidine- 4-carboxamide |
| C92 | 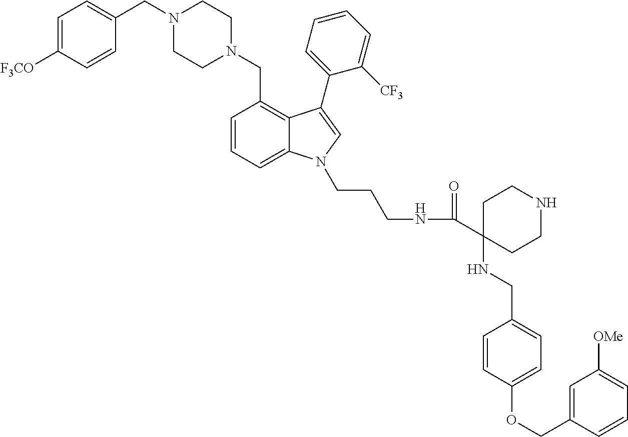 [SMILES: COc1cccc(COc2ccc(CNC3(C(=O)NCCCn4cc(-c5ccccc5C(F)(F)F)c5c(CN6CCN(Cc7ccc(OC(F)(F)F)cc7)CC6)cccc54)CCNCC3)cc2)c1] | 4-(4-(3-methoxybenzyloxy) benzylamino)- N-(3-(4-((4-(4- (trifluoromethoxy)benzyl) piperazin-1-yl)methyl)-3-(2- (trifluoromethyl)phenyl)-1H- indol-1-yl)propyl) piperidine- 4-carboxamide |
| C93 | 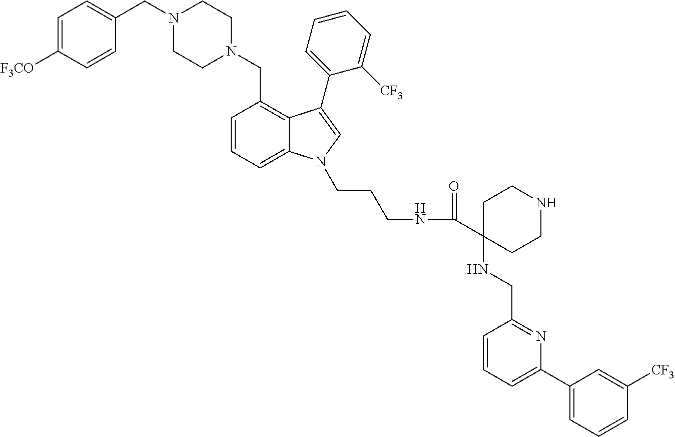 [SMILES: O=C(NCCCn1cc(-c2ccccc2C(F)(F)F)c2c(CN3CCN(Cc4ccc(OC(F)(F)F)cc4)CC3)cccc21)C1(NCc2cccc(-c3cccc(C(F)(F)F)c3)n2)CCNCC1] | N-(3-(4-((4-(4- (trifluoromethoxy)benzyl) piperazin-1-yl)methyl)-3-(2- (trifluoromethyl)phenyl)-1H- indol-1-yl)propyl)-4-((6-(3- (trifluoromethyl)phenyl) pyridin-2- yl)methylamino) piperidine-4- carboxamide |
| C94 | 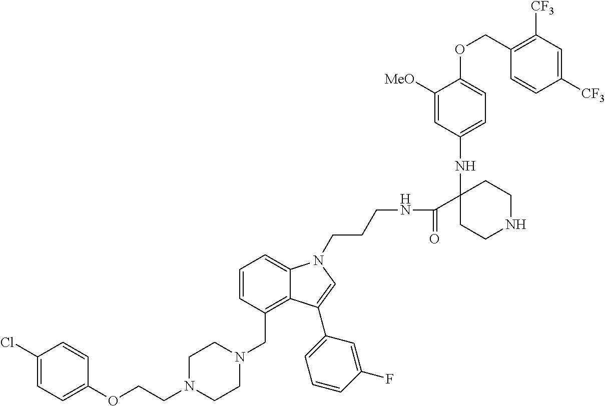 [SMILES: COc1cc(NC2(C(=O)NCCCn3cc(-c4cccc(F)c4)c4c(CN5CCN(CCOc6ccc(Cl)cc6)CC5)cccc43)CCNCC2)ccc1OCc1ccc(C(F)(F)F)cc1C(F)(F)F] | 4-(4-(2,4-bis (trifluoromethyl)benzyloxy)- 3-methoxyphenylamino)-N- (3-(4-((4-(2-(4- chlorophenoxy)ethyl) piperazin- 1-yl)methyl)-3-(2- fluorophenyl)-1H-indol-1- yl)propyl)piperidine-4- carboxamide |
| C95 | 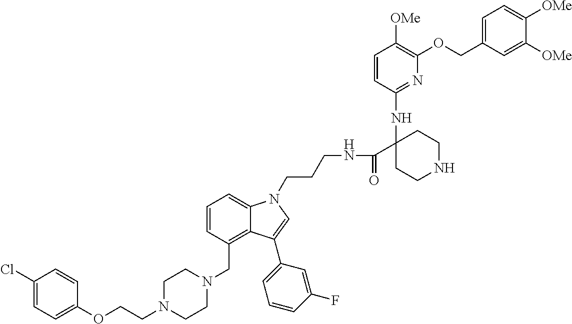 [SMILES: COc1ccc(COc2nc(NC3(C(=O)NCCCn4cc(-c5cccc(F)c5)c5c(CN6CCN(CCOc7ccc(Cl)cc7)CC6)cccc54)CCNCC3)ccc2OC)cc1OC] | N-(3-(4-((4-(2-(4- chlorophenoxy)ethyl) piperazin- 1-yl)methyl)-3-(2- fluorophenyl)-1H-indol-1- yl)propyl)-4-(3-(3,4- dimethoxybenzyloxy)-4- methoxyphenylamino) piperidine- 4-carboxamide |
| C96 | 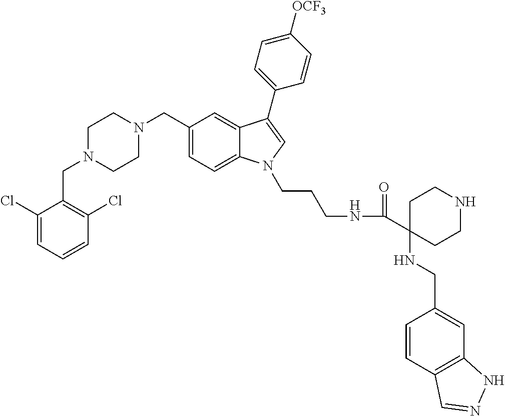 [SMILES: O=C(NCCCn1cc(-c2ccc(OC(F)(F)F)cc2)c2cc(CN3CCN(Cc4c(Cl)cccc4Cl)CC3)ccc21)C1(NCc2ccc3cn[nH]c3c2)CCNCC1] | 4-((1H-indazol-6- yl)methylamino)-N-(3- (5-((4-(2,6- dichlorobenzyl)piperazin-1- yl)methyl)-3-(4- (trifluoromethoxy)phenyl)- 1H-indol-1- yl)propyl)piperidine-4- carboxamide |
| C97 | 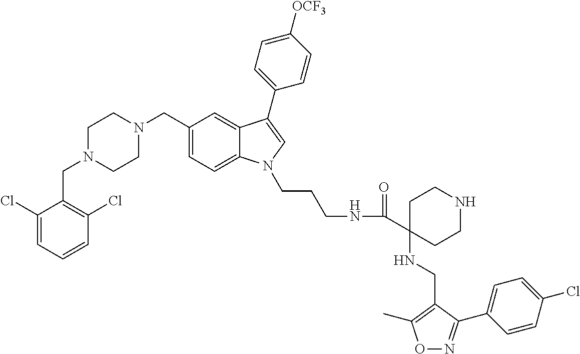 [SMILES: Cc1onc(-c2ccc(Cl)cc2)c1CNC1(C(=O)NCCCn2cc(-c3ccc(OC(F)(F)F)cc3)c3cc(CN4CCN(Cc5c(Cl)cccc5Cl)CC4)ccc32)CCNCC1] | 4-((3-(4-chlorophenyl)-5- methylisoxazol-4- yl)methylamino)-N-(3- (5-((4-(2,6- dichlorobenzyl)piperazin-1- yl)methyl)-3-(4- (trifluoromethoxy)phenyl)- 1H-indol-1- yl)propyl)piperidine-4- carboxamide |
| C98 | 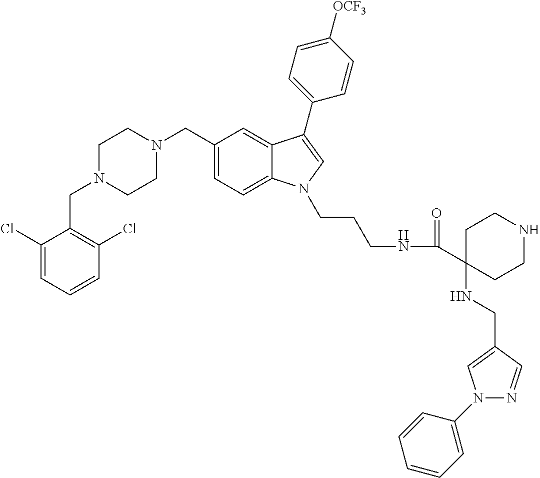 [SMILES: O=C(NCCCn1cc(-c2ccc(OC(F)(F)F)cc2)c2cc(CN3CCN(Cc4c(Cl)cccc4Cl)CC3)ccc21)C1(NCc2cnn(-c3ccccc3)c2)CCNCC1] | N-(3-(5-((4-(2,6- dichlorobenzyl)piperazin-1- yl)methyl)-3-(4- (trifluoromethoxy)phenyl)- 1H-indol-1-yl)propyl)-4-((1- phenyl-1H-pyrazol-3- yl)methylamino) piperidine-4- carboxamide |
| C99 | 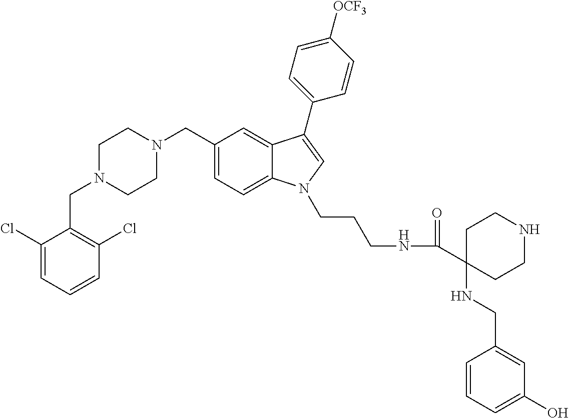 [SMILES: O=C(NCCCn1cc(-c2ccc(OC(F)(F)F)cc2)c2cc(CN3CCN(Cc4c(Cl)cccc4Cl)CC3)ccc21)C1(NCc2cccc(O)c2)CCNCC1] | N-(3-(5-((4-(2,6- dichlorobenzyl)piperazin-1- yl)methyl)-3-(4- (trifluoromethoxy)phenyl)- 1H-indol-1-yl)propyl)-4-(3- hydroxybenzylamino) piperidine- 4-carboxamide |
| C100 | 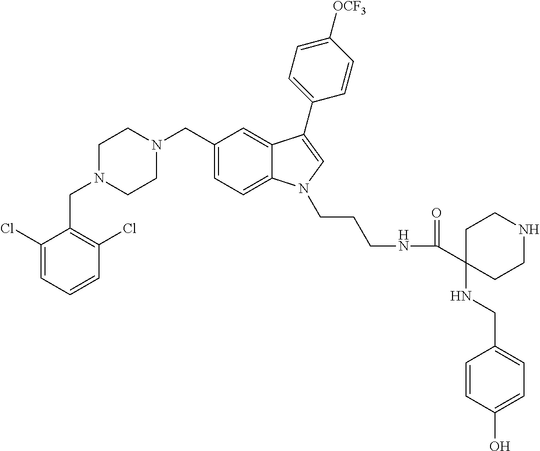 [SMILES: O=C(NCCCn1cc(-c2ccc(OC(F)(F)F)cc2)c2cc(CN3CCN(Cc4c(Cl)cccc4Cl)CC3)ccc21)C1(NCc2ccc(O)cc2)CCNCC1] | N-(3-(5-((4-(2,6- dichlorobenzyl)piperazin-1- yl)methyl)-3-(4- (trifluoromethoxy)phenyl)- 1H-indol-1-yl)propyl)-4-(4- hydroxybenzylamino) piperidine- 4-carboxamide |
| C101 | 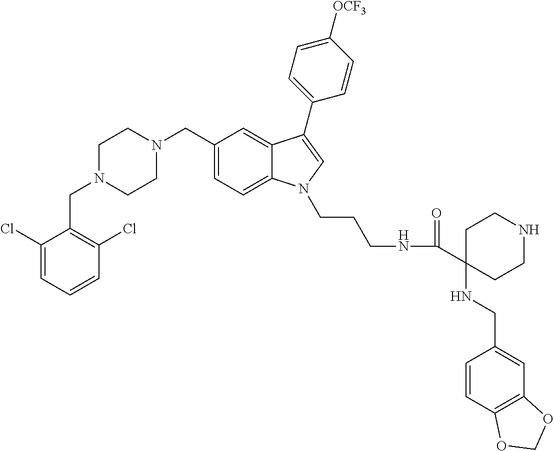 [SMILES: O=C(NCCCn1cc(-c2ccc(OC(F)(F)F)cc2)c2cc(CN3CCN(Cc4c(Cl)cccc4Cl)CC3)ccc21)C1(NCc2ccc3c(c2)OCO3)CCNCC1] | 4-(benzo[d][1,3]dioxol-5- ylmethylamino)-N-(3- (5-((4-(2,6- dichlorobenzyl)piperazin-1- yl)methyl)-3-(4- (trifluoromethoxy)phenyl)- 1H-indol-1- yl)propyl)piperidine-4- carboxamide |
| C102 | 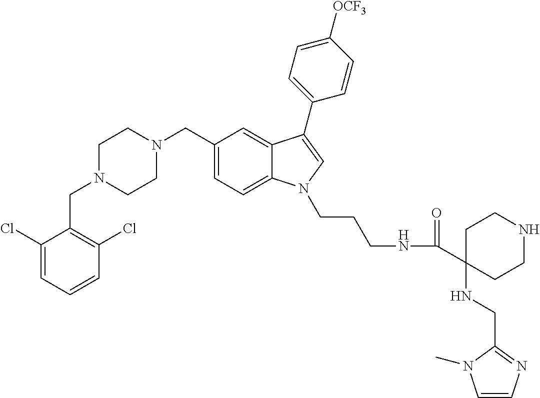 [SMILES: Cn1ccnc1CNC1(C(=O)NCCCn2cc(-c3ccc(OC(F)(F)F)cc3)c3cc(CN4CCN(Cc5c(Cl)cccc5Cl)CC4)ccc32)CCNCC1] | N-(3-(5-((4-(2,6- dichlorobenzyl)piperazin-1- yl)methyl)-3-(4- (trifluoromethoxy)phenyl)- 1H-indol-1-yl)propyl)- 4-((1- methyl-1H-imidazol-2- yl)methylamino) piperidine-4- carboxamide |
| C103 | 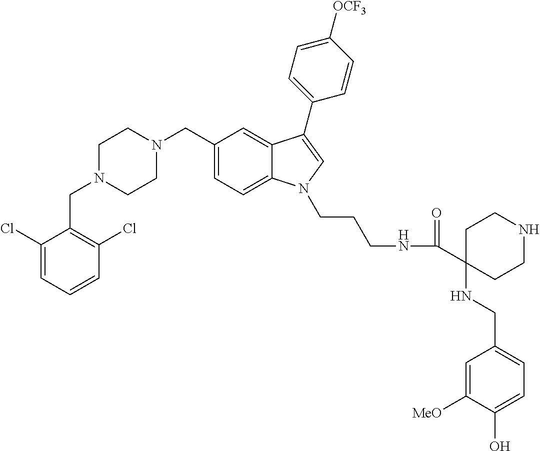 [SMILES: COc1cc(CNC2(C(=O)NCCCn3cc(-c4ccc(OC(F)(F)F)cc4)c4cc(CN5CCN(Cc6c(Cl)cccc6Cl)CC5)ccc43)CCNCC2)ccc1O] | N-(3-(5-((4-(2,6- dichlorobenzyl)piperazin-1- yl)methyl)-3-(4- (trifluoromethoxy)phenyl)- 1H-indol-1-yl)propyl)-4-(4- hydroxy-3- methoxybenzylamino) piperidine- 4-carboxamide |
| C104 | 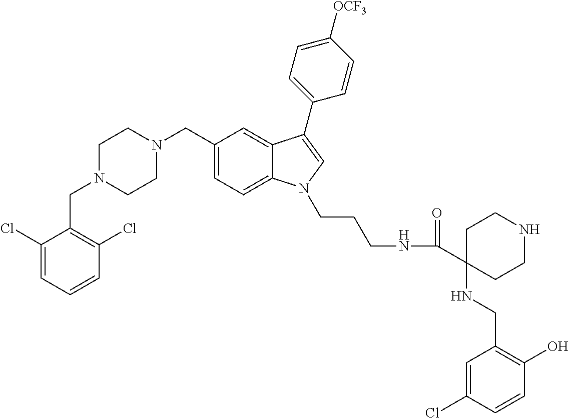 [SMILES: O=C(NCCCn1cc(-c2ccc(OC(F)(F)F)cc2)c2cc(CN3CCN(Cc4c(Cl)cccc4Cl)CC3)ccc21)C1(NCc2cc(Cl)ccc2O)CCNCC1] | 4-(5-chloro-2- hydroxybenzylamino)-N-(3- (5-((4-(2,6- dichlorobenzyl)piperazin-1- yl)methyl)-3-(4- (trifluoromethoxy)phenyl)- 1H-indol-1- yl)propyl)piperidine-4- carboxamide |
| C105 | 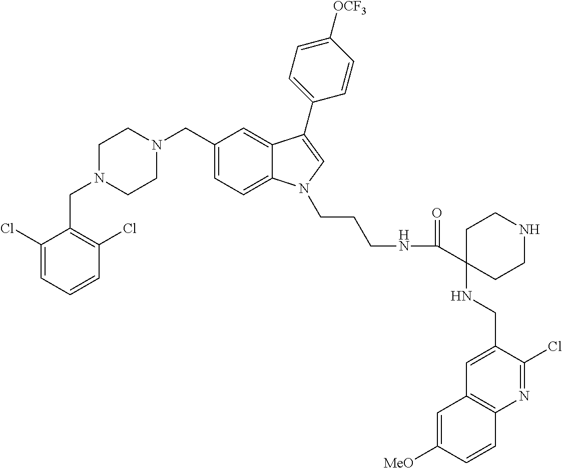 [SMILES: COc1ccc2nc(Cl)c(CNC3(C(=O)NCCCn4cc(-c5ccc(OC(F)(F)F)cc5)c5cc(CN6CCN(Cc7c(Cl)cccc7Cl)CC6)ccc54)CCNCC3)cc2c1] | 4-((2-chloro-6- methoxyquinolin-3- yl)methylamino)-N-(3- (5-((4-(2,6- dichlorobenzyl)piperazin-1- yl)methyl)-3-(4- (trifluoromethoxy)phenyl)- 1H-indol-1- yl)propyl)piperidine-4- carboxamide |
| C106 | 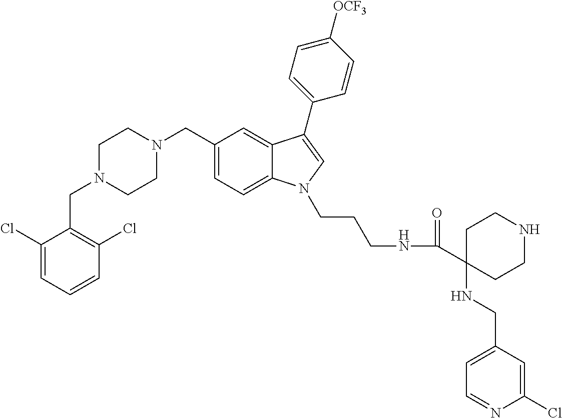 [SMILES: O=C(NCCCn1cc(-c2ccc(OC(F)(F)F)cc2)c2cc(CN3CCN(Cc4c(Cl)cccc4Cl)CC3)ccc21)C1(NCc2ccnc(Cl)c2)CCNCC1] | 4-((2-chloropyridin-4- yl)methylamino)-N-(3- (5-((4-(2,6- dichlorobenzyl)piperazin-1- yl)methyl)-3-(4- (trifluoromethoxy)phenyl)- 1H-indol-1- yl)propyl)piperidine-4- carboxamide |
| C107 | 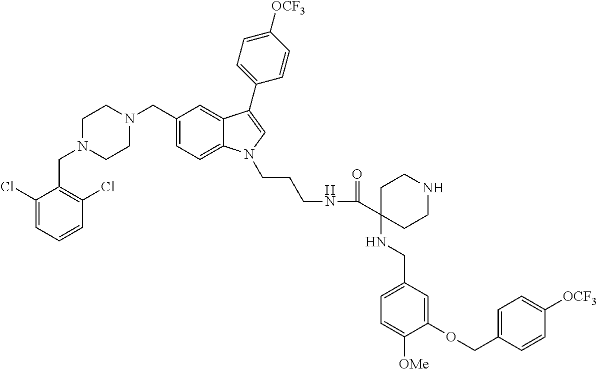 [SMILES: COc1ccc(CNC2(C(=O)NCCCn3cc(-c4ccc(OC(F)(F)F)cc4)c4cc(CN5CCN(Cc6c(Cl)cccc6Cl)CC5)ccc43)CCNCC2)cc1OCc1ccc(OC(F)(F)F)cc1] | N-(3-(5-((4-(2,6- dichlorobenzyl)piperazin-1- yl)methyl)-3-(4- (trifluoromethoxy)phenyl)- 1H-indol-1-yl)propyl)-4-(4- methoxy-3-(4- (trifluoromethoxy) benzyloxy) benzylamino)piperidine-4- carboxamide |
| C109 | 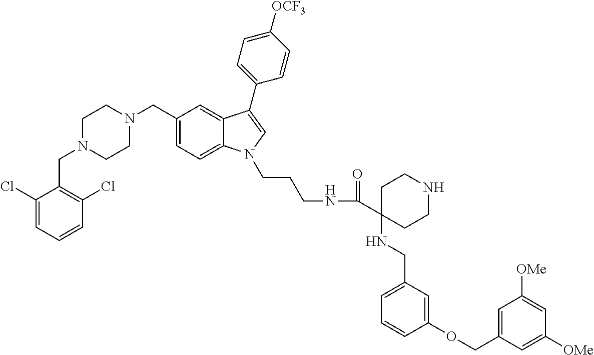 [SMILES: COc1cc(COc2cccc(CNC3(C(=O)NCCCn4cc(-c5ccc(OC(F)(F)F)cc5)c5cc(CN6CCN(Cc7c(Cl)cccc7Cl)CC6)ccc54)CCNCC3)c2)cc(OC)c1] | N-(3-(5-((4-(2,6- dichlorobenzyl)piperazin-1- yl)methyl)-3-(4- (trifluoromethoxy)phenyl)- 1H-indol-1-yl)propyl)-4-(3- (3,5-dimethoxybenzyloxy) benzylamino)piperidine-4- carboxamide |
| C110 | 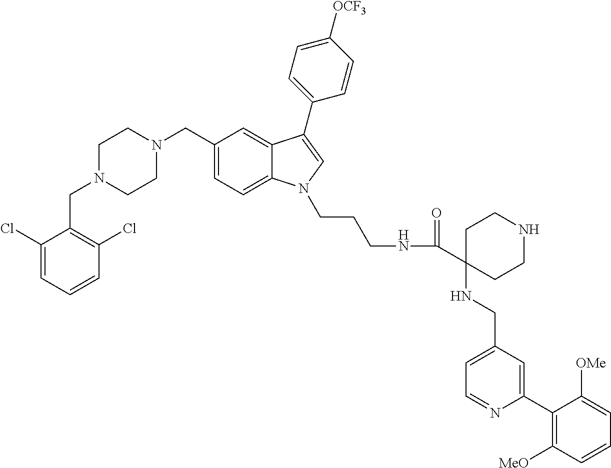 [SMILES: COc1cccc(OC)c1-c1cc(CNC2(C(=O)NCCCn3cc(-c4ccc(OC(F)(F)F)cc4)c4cc(CN5CCN(Cc6c(Cl)cccc6Cl)CC5)ccc43)CCNCC2)ccn1] | N-(3-(5-((4-(2,6- dichlorobenzyl)piperazin-1- yl)methyl)-3-(4- (trifluoromethoxy)phenyl)- 1H-indol-1-yl)propyl)-4-((2- (2,6- dimethoxyphenyl)pyridin-4- yl)methylamino) piperidine-4- carboxamide |
| C111 | 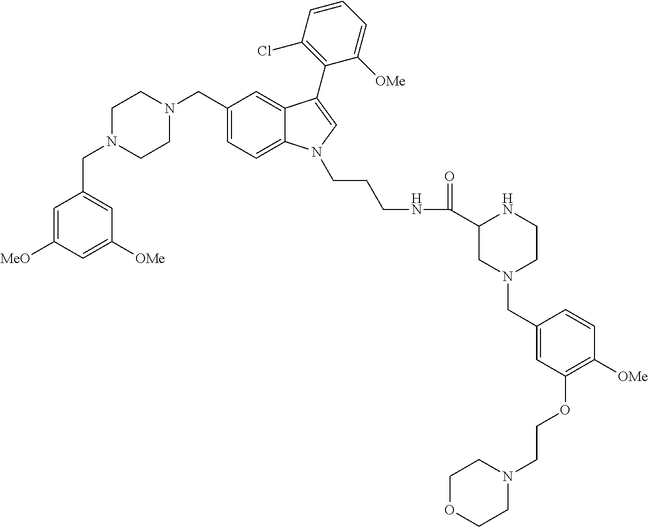 [SMILES: COc1cc(CN2CCN(Cc3ccc4c(c3)c(-c3c(Cl)cccc3OC)cn4CCCNC(=O)C3CN(Cc4ccc(OC)c(OCCN5CCOCC5)c4)CCN3)CC2)cc(OC)c1] | N-(3-(3-(2-chloro-6- methoxyphenyl)-5-((4-(3,5- dimethoxybenzyl) piperazin-1- yl)methyl)-1H-indol-1- yl)propyl)-4-(4- methoxy-3-(2- morpholinoethoxy)benzyl) piperazine-2-carboxamide |
| C112 | 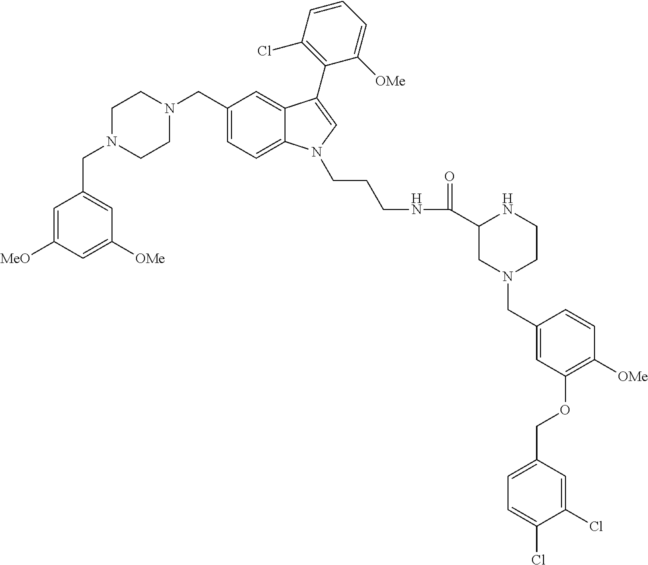 [SMILES: COc1cc(CN2CCN(Cc3ccc4c(c3)c(-c3c(Cl)cccc3OC)cn4CCCNC(=O)C3CN(Cc4ccc(OC)c(OCc5ccc(Cl)c(Cl)c5)c4)CCN3)CC2)cc(OC)c1] | N-(3-(3-(2-chloro-6- methoxyphenyl)-5-((4-(3,5- dimethoxybenzyl) piperazin-1- yl)methyl)-1H-indol-1- yl)propyl)-4-(3-(3,4- dichlorobenzyloxy)-4- methoxybenzyl) piperazine-2- carboxamide |
| C115 | 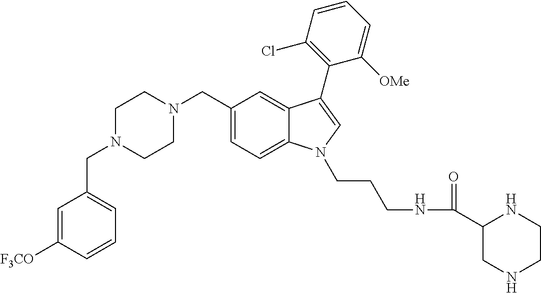 [SMILES: COc1cccc(Cl)c1-c1cn(CCCNC(=O)C2CNCCN2)c2ccc(CN3CCN(Cc4cccc(OC(F)(F)F)c4)CC3)cc12] | N-(3-(3-(2-chloro-6- methoxyphenyl)-5-((4-(3- (trifluoromethoxy)benzyl) piperazin-1-yl)methyl)-1H- indol-1-yl)propyl) piperazine-2- carboxamide |
| C116 | 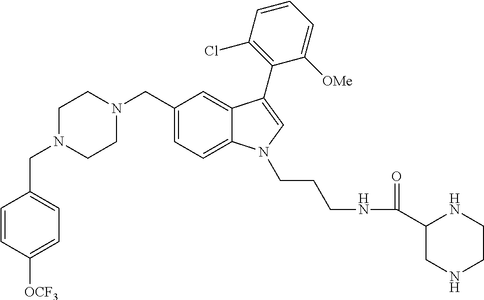 [SMILES: COc1cccc(Cl)c1-c1cn(CCCNC(=O)C2CNCCN2)c2ccc(CN3CCN(Cc4ccc(OC(F)(F)F)cc4)CC3)cc12] | N-(3-(3-(2-chloro-6- methoxyphenyl)-5-((4-(4- (trifluoromethoxy)benzyl) piperazin-1-yl)methyl)-1H- indol-1-yl)propyl) piperazine-2- carboxamide |
| C117 | 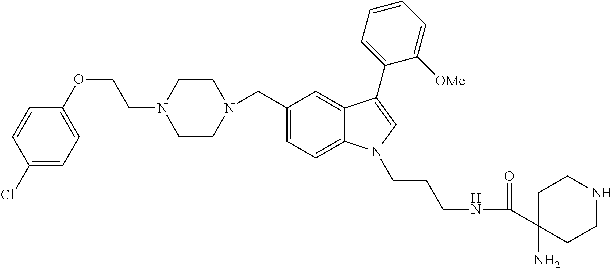 [SMILES: COc1ccccc1-c1cn(CCCNC(=O)C2(N)CCNCC2)c2ccc(CN3CCN(CCOc4ccc(Cl)cc4)CC3)cc12] | 4-amino-N-(3-(5-((4-(2-(4- chlorophenoxy)ethyl) piperazin- 1-yl)methyl)-3-(2- methoxyphenyl)-1H- indol-1- yl)propyl)piperidine-4- carboxamide |
| C118 | 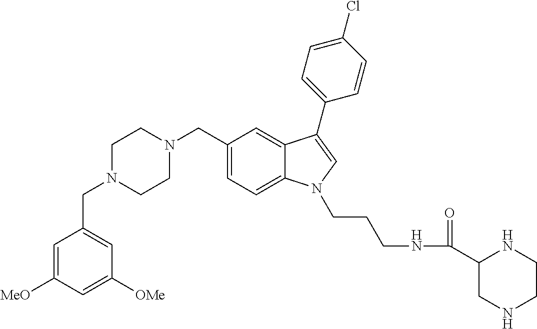 [SMILES: COc1cc(CN2CCN(Cc3ccc4c(c3)c(-c3ccc(Cl)cc3)cn4CCCNC(=O)C3CNCCN3)CC2)cc(OC)c1] | N-(3-(3-(4-chlorophenyl)-5- ((4-(3,5- dimethoxybenzyl) piperazin-1- yl)methyl)-1H-indol-1- yl)propyl)piperazine-2- carboxamide |
| C119 | 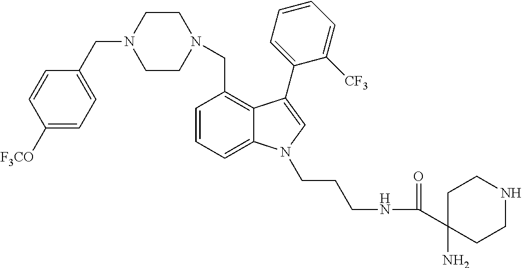 [SMILES: NC1(C(=O)NCCCn2cc(-c3ccccc3C(F)(F)F)c3c(CN4CCN(Cc5ccc(OC(F)(F)F)cc5)CC4)cccc32)CCNCC1] | 4-amino-N-(3-(4-((4-(4- (trifluoromethoxy)benzyl) piperazin-1-yl)methyl)-3-(2- (trifluoromethyl)phenyl)-1H- indol-1-yl)propyl) piperidine- 4-carboxamide |
| C120 | 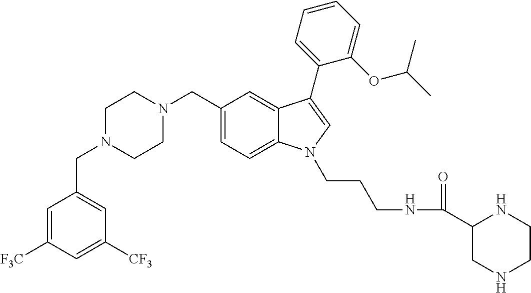 [SMILES: CC(C)Oc1ccccc1-c1cn(CCCNC(=O)C2CNCCN2)c2ccc(CN3CCN(Cc4cc(C(F)(F)F)cc(C(F)(F)F)c4)CC3)cc12] | N-(3-(5-((4-(3,5- bis(trifluoromethyl)benzyl) piperazin-1-yl)methyl)-3-(2- isopropoxyphenyl)- 1H-indol- 1-yl)propyl)piperazine-2- carboxamide |
| C121 | 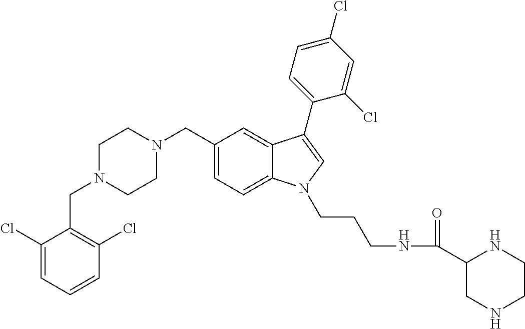 [SMILES: O=C(NCCCn1cc(-c2ccc(Cl)cc2Cl)c2cc(CN3CCN(Cc4c(Cl)cccc4Cl)CC3)ccc21)C1CNCCN1] | N-(3-(5-((4-(2,6- dichlorobenzyl)piperazin-1- yl)methyl)-3-(2,4- dichlorophenyl)-1H-indol-1- yl)propyl)piperazine-2- carboxamide |
| C124 | 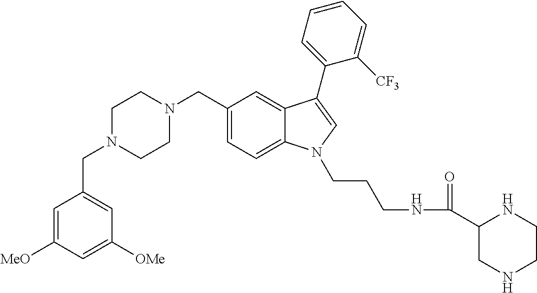 [SMILES: COc1cc(CN2CCN(Cc3ccc4c(c3)c(-c3ccccc3C(F)(F)F)cn4CCCNC(=O)C3CNCCN3)CC2)cc(OC)c1] | N-(3-(5-((4-(3,5- dimethoxybenzyl) piperazin-1- yl)methyl)-3-(2- (trifluoromethyl) phenyl)-1H- indol-1-yl)propyl) piperazine- 2-carboxamide |
| C125 | 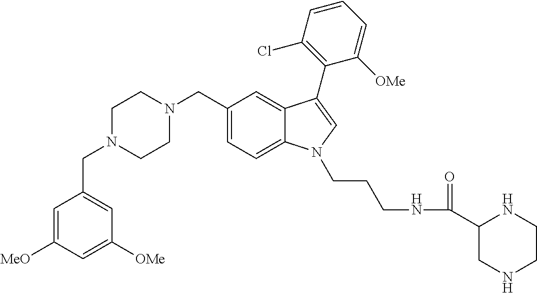 [SMILES: COc1cc(CN2CCN(Cc3ccc4c(c3)c(-c3c(Cl)cccc3OC)cn4CCCNC(=O)C3CNCCN3)CC2)cc(OC)c1] | N-(3-(3-(2-chloro-6- methoxyphenyl)-5-((4-(3,5- dimethoxybenzyl) piperazin-1- yl)methyl)-1H-indol-1- yl)propyl)piperazine-2- carboxamide |
| C126 | 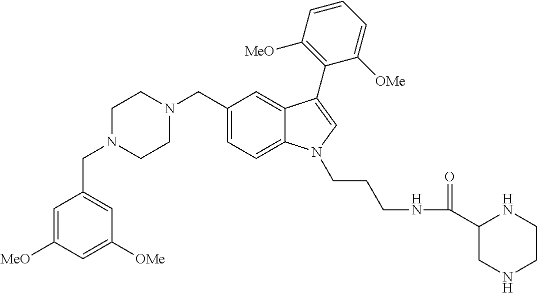 [SMILES: COc1cc(CN2CCN(Cc3ccc4c(c3)c(-c3c(OC)cccc3OC)cn4CCCNC(=O)C3CNCCN3)CC2)cc(OC)c1] | N-(3-(5-((4-(3,5- dimethoxybenzyl) piperazin-1- yl)methyl)-3-(2,6- dimethoxyphenyl)- 1H-indol- 1-yl)propyl)piperazine-2- carboxamide |
| C127 | 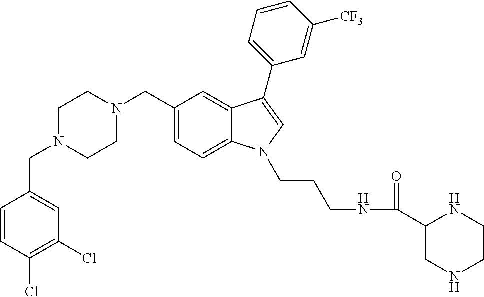 [SMILES: O=C(NCCCn1cc(-c2cccc(C(F)(F)F)c2)c2cc(CN3CCN(Cc4ccc(Cl)c(Cl)c4)CC3)ccc21)C1CNCCN1] | N-(3-(5-((4-(3,4- dichlorobenzyl)piperazin-1- yl)methyl)-3-(3- (trifluoromethyl) phenyl)-1H- indol-1-yl)propyl) piperazine- 2-carboxamide |
| C170 | 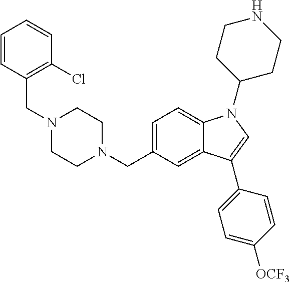 [SMILES: FC(F)(F)Oc1ccc(-c2cn(C3CCNCC3)c3ccc(CN4CCN(Cc5ccccc5Cl)CC4)cc23)cc1] | 5-((4-(2- chlorobenzyl)piperazin-1- yl)methyl)-1- (piperidin-4-yl)- 3-(4- (trifluoromethoxy)phenyl)- 1H-indole |
| C171 | 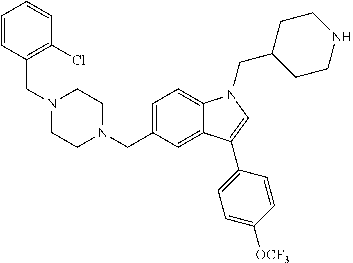 [SMILES: FC(F)(F)Oc1ccc(-c2cn(CC3CCNCC3)c3ccc(CN4CCN(Cc5ccccc5Cl)CC4)cc23)cc1] | 5-((4-(2- chlorobenzyl)piperazin-1- yl)methyl)-1-(piperidin-4- ylmethyl)-3-(4- (trifluoromethoxy)phenyl)- 1H-indole |
| C172 | 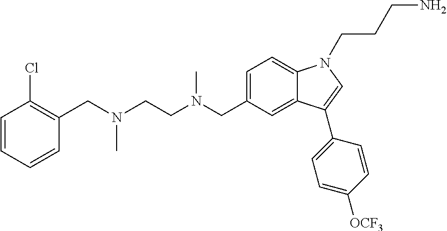 [SMILES: CN(CCN(C)Cc1ccccc1Cl)Cc1ccc2c(c1)c(-c1ccc(OC(F)(F)F)cc1)cn2CCCN] | N1-((1-(3-aminopropyl)- 3-(4- (trifluoromethoxy )phenyl)- 1H-indol-5-yl)methyl)- N2-(2- chlorobenzyl)-N1,N2- dimethylethane-1,2-diamine |
| C173 | 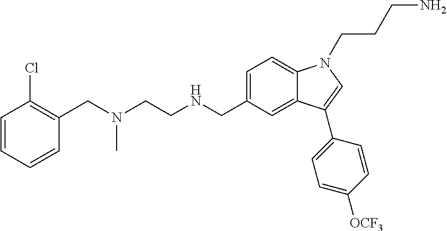 [SMILES: CN(CCNCc1ccc2c(c1)c(-c1ccc(OC(F)(F)F)cc1)cn2CCCN)Cc1ccccc1Cl] | N1-((1-(3-aminopropyl)- 3-(4-(trifluoromethoxy) phenyl)- 1H-indol-5-yl)methyl)- N2-(2-chlorobenzyl)-N2- methylethane-1,2-diamine |

In some embodiments, the compound is selected from any one of the compounds from the following table:

TABLE 6B

| Compound | | |
|---|---|---|
| Ref. | Compound | Chemical Name |
| C193 | 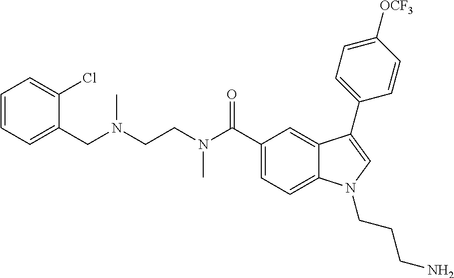 [SMILES: CN(CCN(C)C(=O)c1ccc2c(c1)c(-c1ccc(OC(F)(F)F)cc1)cn2CCCN)Cc1ccccc1Cl] | 1-(3-aminopropyl)-N-(2-((2- chlorobenzyl)(methyl)amino) ethyl)-N-methyl-3-(4- (trifluoromethoxy)phenyl)- 1H-indole-5-carboxamide |
| C197 | 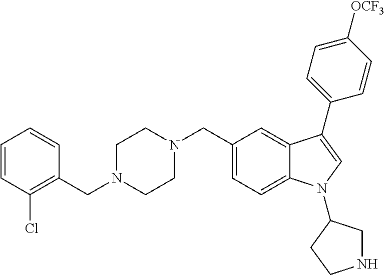 [SMILES: FC(F)(F)Oc1ccc(-c2cn(C3CCNC3)c3ccc(CN4CCN(Cc5ccccc5Cl)CC4)cc23)cc1] | 5-((4-(2- chlorobenzyl)piperazin-1- yl)methyl)-1-(pyrrolidn-3- yl)-3-(4- (trifluoromethoxy)phenyl)- 1H-indole |
| C198 | 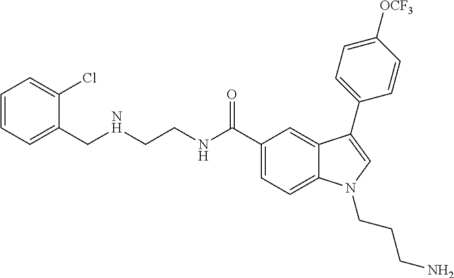 [SMILES: NCCCn1cc(-c2ccc(OC(F)(F)F)cc2)c2cc(C(=O)NCCNCc3ccccc3Cl)ccc21] | N1-((1-(3-aminopropyl)-3-(4- (trifluoromethoxy)phenyl)- 1H-indol-5-yl)methyl)-N2-(2- chlorobenzyl)ethane-1,2- diamine |
| C203 | 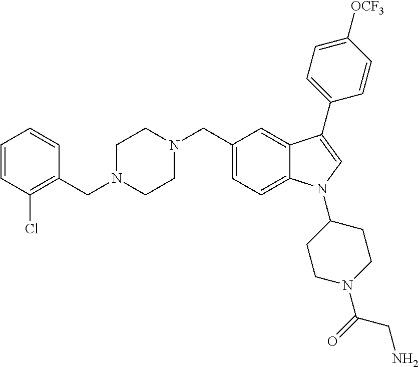 [SMILES: NCC(=O)N1CCC(n2cc(-c3ccc(OC(F)(F)F)cc3)c3cc(CN4CCN(Cc5ccccc5Cl)CC4)ccc32)CC1] | 2-amino-1-(4-(5-((4-(2- chlorobenzyl)piperazin-1- yl)methyl)-3-(4- (trifluoromethoxy)phenyl)- 1H-indol-1-yl)piperidin-1- yl)ethan-1-one |
| C209 | 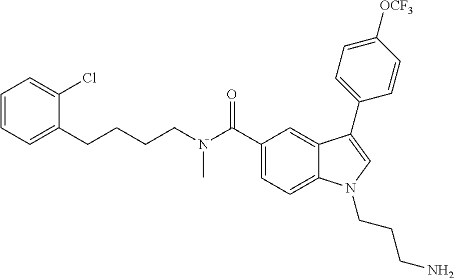 [SMILES: CN(CCCCc1ccccc1Cl)C(=O)c1ccc2c(c1)c(-c1ccc(OC(F)(F)F)cc1)cn2CCCN] | N-((1-(3-aminopropyl)-3-(4- (trifluoromethoxy)phenyl)- 1H-indol-5-yl)methyl)-4-(2- chlorophenyl)-N- methylbutan-1-amine |
| C217 | 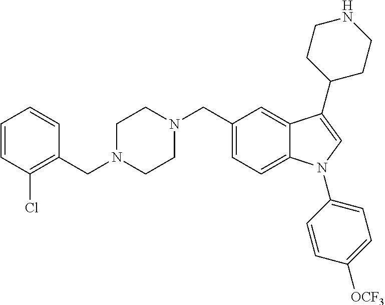 [SMILES: FC(F)(F)Oc1ccc(-n2cc(C3CCNCC3)c3cc(CN4CCN(Cc5ccccc5Cl)CC4)ccc32)cc1] | 5-((4-(2- chlorobenzyl)piperazin-1- yl)methyl)-3-(piperidin-4-yl)- 1-(4- (trifluoromethoxy)phenyl)- 1H-indole |
| C218 | 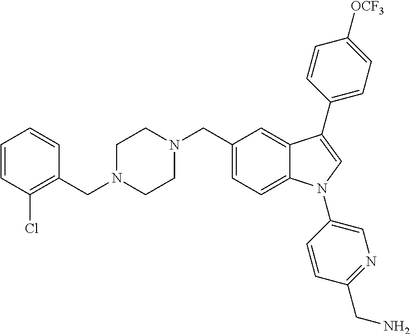 [SMILES: NCc1ccc(-n2cc(-c3ccc(OC(F)(F)F)cc3)c3cc(CN4CCN(Cc5ccccc5Cl)CC4)ccc32)cn1] | (5-(5-((4-(2- chlorobenzyl)piperazin-1- yl)methyl)-3-(4- (trifluoromethoxy)phenyl)- 1H-indol-1-yl)pyridin-2- yl)methanamine |
| C219 | 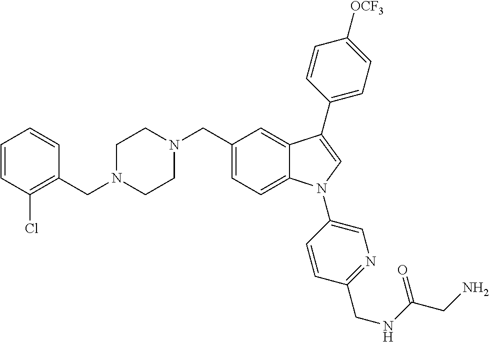 [SMILES: NCC(=O)NCc1ccc(-n2cc(-c3ccc(OC(F)(F)F)cc3)c3cc(CN4CCN(Cc5ccccc5Cl)CC4)ccc32)cn1] | 2-amino-N-((5-(5-((4-(2- chlorobenzyl)piperazin-1- yl)methyl)-3-(4- (trifluoromethoxy)phenyl)- 1H-indol-1-yl)pyridin-2- yl)methyl)acetamide |
| C220 | 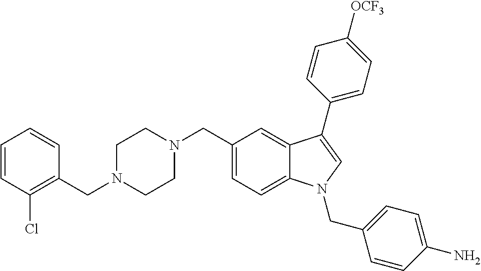 [SMILES: Nc1ccc(Cn2cc(-c3ccc(OC(F)(F)F)cc3)c3cc(CN4CCN(Cc5ccccc5Cl)CC4)ccc32)cc1] | 4-((5-((4-(2- chlorobenzyl)piperazin-1- yl)methyl)-3-(4- (trifluoromethoxy)phenyl)- 1H-indol-1-yl)methyl)aniline |
| C224 | 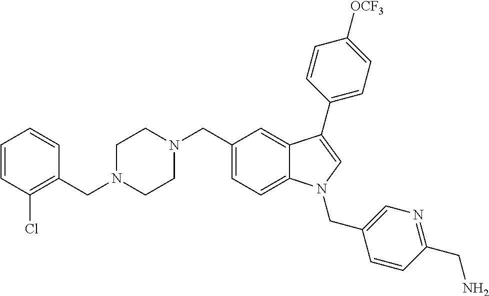 [SMILES: NCc1ccc(Cn2cc(-c3ccc(OC(F)(F)F)cc3)c3cc(CN4CCN(Cc5ccccc5Cl)CC4)ccc32)cn1] | (5-((5-((4-(2- chlorobenzyl)piperazin-1- yl)methyl)-3-(4- (trifluoromethoxy)phenyl)- 1H-indol-1-yl)methyl)pyridin- 2-yl)methanamine |
| C225 | 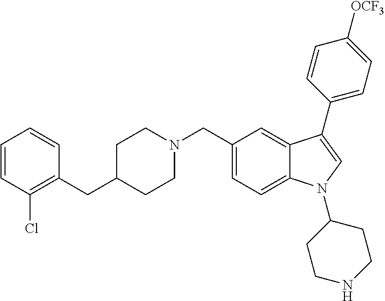 [SMILES: FC(F)(F)Oc1ccc(-c2cn(C3CCNCC3)c3ccc(CN4CCC(Cc5ccccc5Cl)CC4)cc23)cc1] | 5-((4-(2- chlorobenzyl)piperidin-1- yl)methyl)-1-(piperidin-4-yl)- 3-(4- (trifluoromethoxy)phenyl)- 1H-indole |
| C227 | 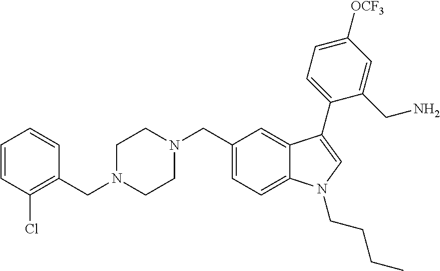 [SMILES: CCCCn1cc(-c2ccc(OC(F)(F)F)cc2CN)c2cc(CN3CCN(Cc4ccccc4Cl)CC3)ccc21] | (2-(1-butyl-5-((4-(2- chlorobenzyl)piperazin-1- yl)methyl)-1H-indol-3-yl)-5- (trifluoromethoxy)phenyl) methanamine |
| C228 | 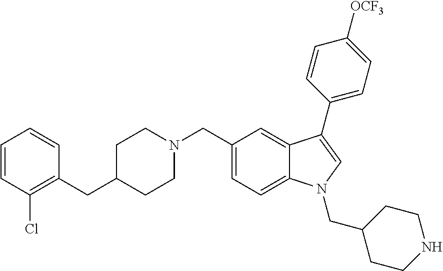 [SMILES: FC(F)(F)Oc1ccc(-c2cn(CC3CCNCC3)c3ccc(CN4CCC(Cc5ccccc5Cl)CC4)cc23)cc1] | 5-((4-(2- chlorobenzyl)piperidin-1- yl)methyl)-1-(piperidin-4- ylmethyl)-3-(4- (trifluoromethoxy)phenyl)- 1H-indole |
| C229 | 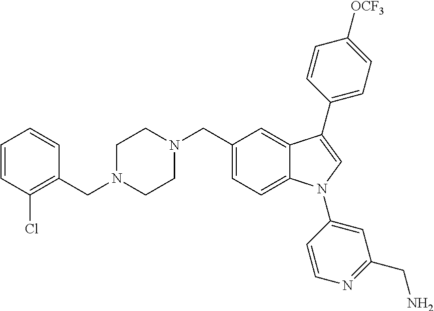 [SMILES: NCc1cc(-n2cc(-c3ccc(OC(F)(F)F)cc3)c3cc(CN4CCN(Cc5ccccc5Cl)CC4)ccc32)ccn1] | (4-(5-((4-(2- chlorobenzyl)piperazin-1- yl)methyl)-3-(4- (trifluoromethoxy)phenyl)- 1H-indol-1-yl)pyridin-2- yl)methanamine |
| C230 | 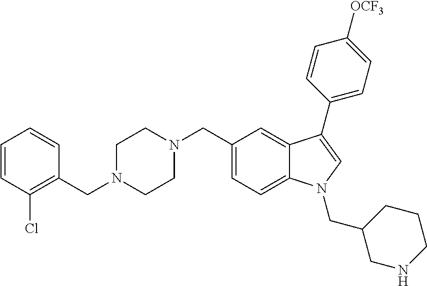 [SMILES: FC(F)(F)Oc1ccc(-c2cn(CC3CCCNC3)c3ccc(CN4CCN(Cc5ccccc5Cl)CC4)cc23)cc1] | 5-((4-(2- chlorobenzyl)piperazin-1- yl)mehtyl)-1-(piperidin-3- ylmethyl)-3-(4- (trifluoromethoxy)phenyl)- 1H-indole |
| C231 | 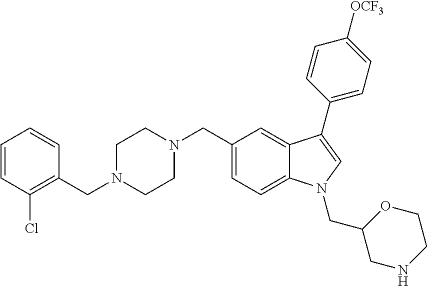 [SMILES: FC(F)(F)Oc1ccc(-c2cn(CC3CNCCO3)c3ccc(CN4CCN(Cc5ccccc5Cl)CC4)cc23)cc1] | 2-((5-((4-(2- chlorobenzyl)piperazin-1- yl)methyl)-3-(4- (trifluoromethoxy)phenyl)- 1H-indol-1- yl)methyl)morpholine |
| C232 | 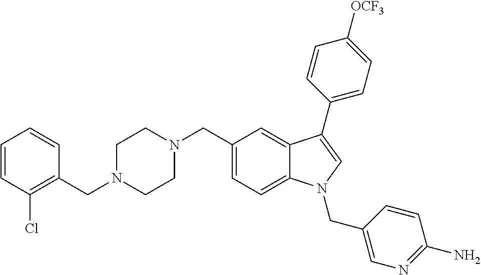 [SMILES: Nc1ccc(Cn2cc(-c3ccc(OC(F)(F)F)cc3)c3cc(CN4CCN(Cc5ccccc5Cl)CC4)ccc32)cn1] | 5-((5-((4-(2- chlorobenzyl)piperazin-1- yl)methyl)-3-(4- (trifluoromethoxy)phenyl)- 1H-indol-1-yl)methyl)pyridin- 2-amine |
| C234 | 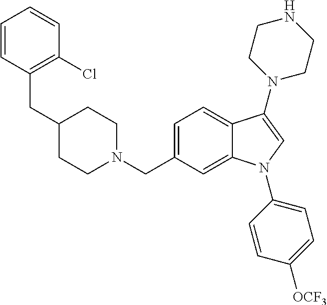 [SMILES: FC(F)(F)Oc1ccc(-n2cc(N3CCNCC3)c3ccc(CN4CCC(Cc5ccccc5Cl)CC4)cc32)cc1] | 6-((4-(2- chlorobenzyl)piperidin-1- yl)methyl)-3-(piperazin-1-yl)- 1-(4- (trifluoromethoxy)phenyl)- 1H-indole |
| C235 | 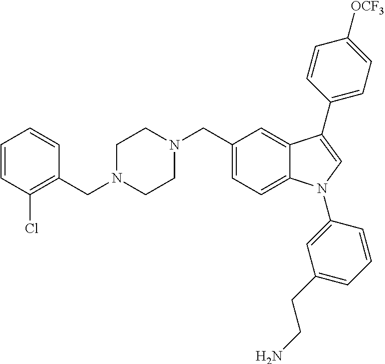 [SMILES: NCCc1cccc(-n2cc(-c3ccc(OC(F)(F)F)cc3)c3cc(CN4CCN(Cc5ccccc5Cl)CC4)ccc32)c1] | 2-(3-(5-((4-(2- chlorobenzyl)piperazin-1- yl)methyl)-3-(4- (trifluoromethoxy)phenyl)- 1H-indol-1-yl)phenyl)ethan- 1-amine |
| C237 | 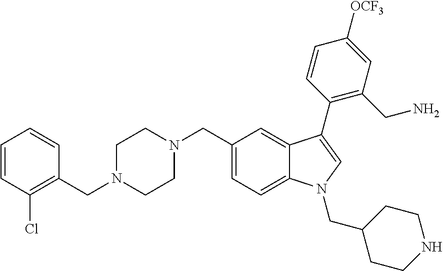 [SMILES: NCc1cc(OC(F)(F)F)ccc1-c1cn(CC2CCNCC2)c2ccc(CN3CCN(Cc4ccccc4Cl)CC3)cc12] | (2-(5-((4-(2- chlorobenzyl)piperazin-1- yl)methyl)-1-(piperidin-4- ylmethyl)-1H-indol-3-yl)-5- (trifluoromethoxy)phenyl) methanamine |
| C243 | 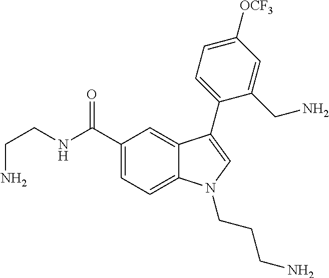 [SMILES: NCCCn1cc(-c2ccc(OC(F)(F)F)cc2CN)c2cc(C(=O)NCCN)ccc21] | N1-((3-(2-(aminomethyl)-4- (trifluoromethoxy)phenyl)-1- (3-aminopropyl)-1H-indol-5- yl)methyl)ethane-1,2-diamine |
| C246 | 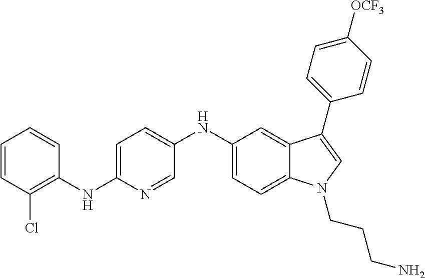 [SMILES: NCCCn1cc(-c2ccc(OC(F)(F)F)cc2)c2cc(Nc3ccc(Nc4ccccc4Cl)nc3)ccc21] | N5-(1-(3-aminopropyl)-3-(4- (trifluoromethoxy)phenyl)- 1H-indol-5-yl)-N2-(2- chlorophenyl)pyridine-2,5- diamine |
| C248 | 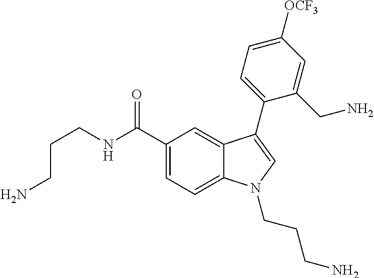 [SMILES: NCCCNC(=O)c1ccc2c(c1)c(-c1ccc(OC(F)(F)F)cc1CN)cn2CCCN] | N1-((3-(2-(aminomethyl)-4- (trifluoromethoxy)phenyl)-1- (3-aminopropyl)-1H-indol-5- yl)methyl)propane-1,3- diamine |
| C254 | 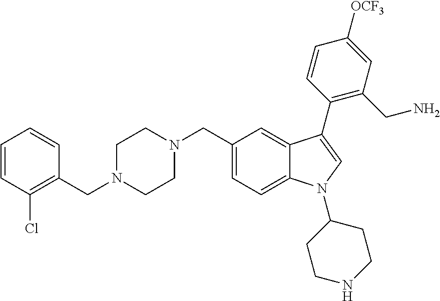 [SMILES: NCc1cc(OC(F)(F)F)ccc1-c1cn(C2CCNCC2)c2ccc(CN3CCN(Cc4ccccc4Cl)CC3)cc12] | (2-(5-((4-(2- chlorobenzyl)piperazin-1- yl)methyl)-1-(piperidin-4-yl)- 1H-indol-3-yl)-5- (trifluoromethoxy)phenyl) methanamine |
| C264 | 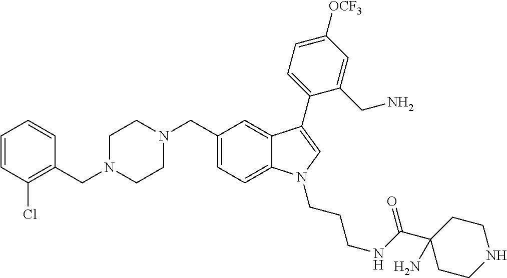 [SMILES: NCc1cc(OC(F)(F)F)ccc1-c1cn(CCCNC(=O)C2(N)CCNCC2)c2ccc(CN3CCN(Cc4ccccc4Cl)CC3)cc12] | 4-amino-N-(3-(3-(2- (aminomethyl)-4- (trifluoromethoxy)phenyl)-5- ((4-(2- chlorobenzyl)piperazin-1- yl)methyl)-1H-indol-1- yl)propyl)piperidine-4- carboxamide |
| C268 | 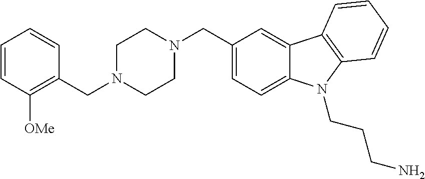 [SMILES: COc1ccccc1CN1CCN(Cc2ccc3c(c2)c2ccccc2n3CCCN)CC1] | 3-(3-((4-(2- methoxybenzyl)piperazin-1- yl)methyl)-9H-carbazol-9- yl)propan-1-amine |
| C274 | 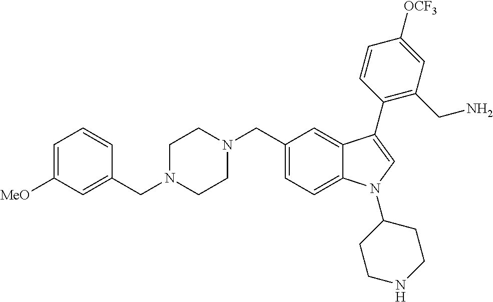 [SMILES: COc1cccc(CN2CCN(Cc3ccc4c(c3)c(-c3ccc(OC(F)(F)F)cc3CN)cn4C3CCNCC3)CC2)c1] | (2-(5-((4-(3- methoxybenzyl)piperazin-1- yl)methyl)-1-(piperidin-4-yl)- 1H-indol-3-yl)-5- (trifluoromethoxy)phenyl) methanamine |
| C275 | 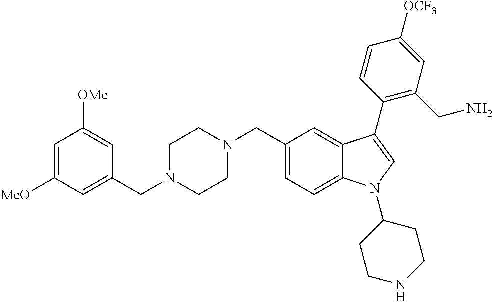 [SMILES: COc1cc(CN2CCN(Cc3ccc4c(c3)c(-c3ccc(OC(F)(F)F)cc3CN)cn4C3CCNCC3)CC2)cc(OC)c1] | (2-(5-((4-(3,5- dimethoxybenzyl)piperazin-1- yl)methyl)-1-(piperidin-4-yl)- 1H-i9ndol-3-yl)-5- (trifluoromethoxy)phenyl) methanamine |
| C296 | 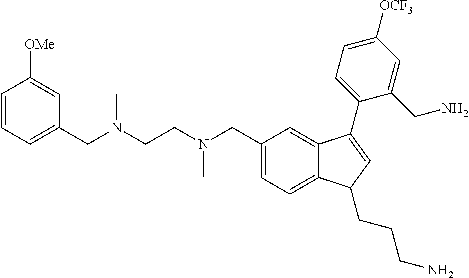 [SMILES: COc1cccc(CN(C)CCN(C)Cc2ccc3c(c2)C(c2ccc(OC(F)(F)F)cc2CN)=CC3CCCN)c1] | N1-((3-(2-(aminomethyl)-4- (trifluoromethoxy)phenyl)-1- (3-aminopropyl)-1H-indol-5- yl)methyl)-N2-(3- methoxybenzyl)-N1,N2- dimethylethane-1,2-diamine |
| C297 | 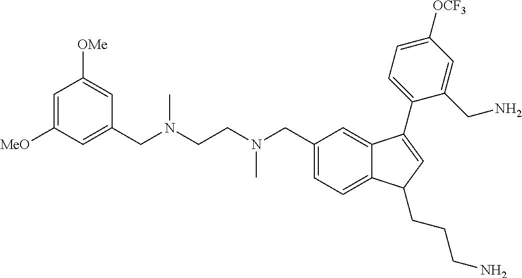 [SMILES: COc1cc(CN(C)CCN(C)Cc2ccc3c(c2)C(c2ccc(OC(F)(F)F)cc2CN)=CC3CCCN)cc(OC)c1] | N1-((3-(2-(aminomethyl)-4- (trifluoromethoxy)phenyl)-1- (3-aminopropyl)-1H-indol-5- yl)methyl)-N2-(3,5- dimethoxybenzyl)-N1,N2- dimethylethane-1,2-diamine |
| C298 | 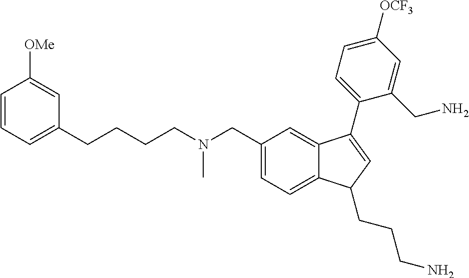 [SMILES: COc1cccc(CCCCN(C)Cc2ccc3c(c2)C(c2ccc(OC(F)(F)F)cc2CN)=CC3CCCN)c1] | N-((3-(2-(aminomethyl)-4- (trifluoromethoxy)phenyl)-1- (3-aminopropyl)-1H-indol-5- yl)methyl)-4-(3- methoxyphenyl)-N- methylbutan-1-amine |

In another aspect, provided herein is a compound of Formula (IIIa), or a pharmaceutically acceptable salt, or solvate thereof:

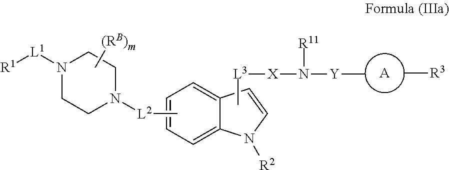

wherein,
L¹ and L² are each independently an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is an optionally substituted aryl, optionally substituted heterocycloalkyl, or optionally substituted heteroaryl;
R² is an optionally substituted aryl, optionally substituted heterocycloalkyl, or optionally substituted heteroaryl;
each R^{B} is independently optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N; wherein if Ring A is substituted, then Ring A is substituted with at least one R^{A};
each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring,
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); and
each m is independently 0, 1, 2, 3, or 4.

In some embodiments, the compound has the following structure of Formula (IIIb), or a pharmaceutically acceptable salt, or solvate thereof:

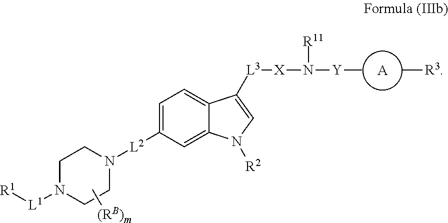

In some embodiments, the compound has the following structure of Formula (IIIc), or a pharmaceutically acceptable salt, or solvate thereof:

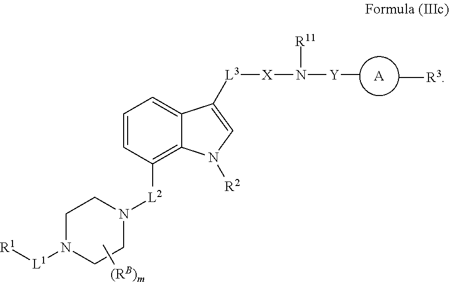

In some embodiments,

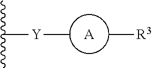

is selected from the following:

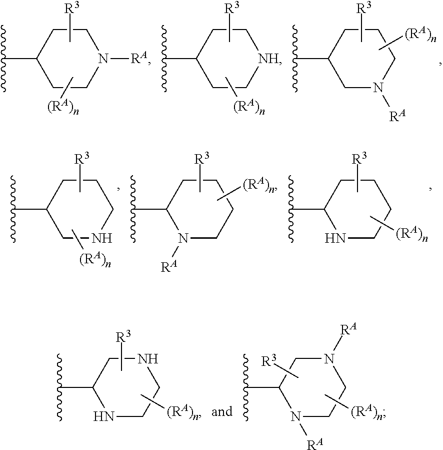

and each n is independently 0, 1, 2, 3, or 4.

In some embodiments,

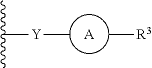

is selected from the following:

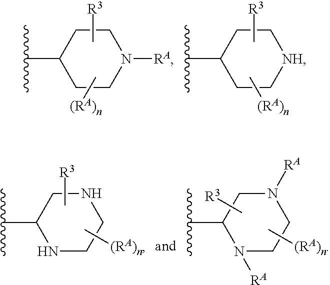

In some embodiments,

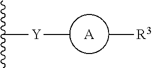

is selected from the following:

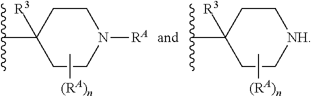

In some embodiments, L¹ is —CH₂—, C(═O), O, S, S(═O), S(═O)₂, or NR⁴. In some embodiments, L¹ is —CH_{2—}. In some embodiments, L² is —CH₂—, C(═O), O, S, S(═O), S(═O)₂, or NR⁴. In some embodiments, L² is —CH₂—. In some embodiments, L³ is absent, —CH₂—, —CH₂—CH₂—, or —CH₂—CH₂—CH₂—. In some embodiments, L³ is —CH₂—CH₂—.

In some embodiments, L³ is:

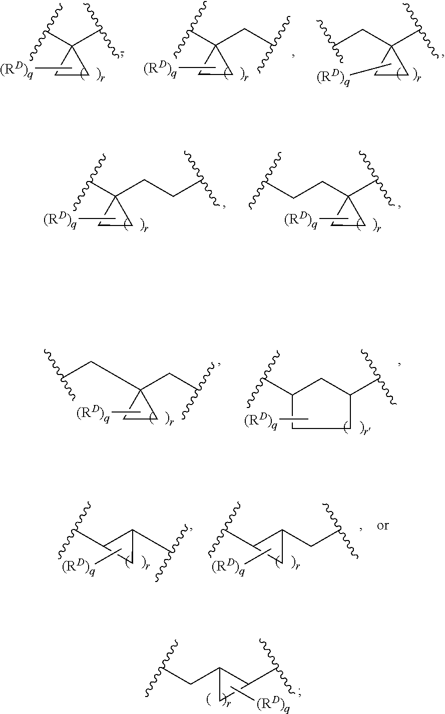

each q is independently 0, 1, 2, 3, or 4;

r is 1, 2, 3, or 4; and

r′ is 1 or 2.

In some embodiments, X is —CH₂— or C(═O). In some embodiments, X is —CH₂—.

In some embodiments, X is:

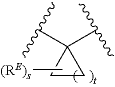

each s is independently 0, 1, 2, 3, or 4; and

t is 1, 2, 3, or 4.

In some embodiments, X is:

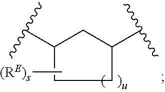

each s is independently 0, 1, 2, 3, or 4; and

u is 0, 1, or 2.

In some embodiments, L³-X is —CH₂—CH₂—CH₂—. In some embodiments, Y is —CH₂— or C(═O). In some embodiments, Y is C(═O). In some embodiments, R¹¹ is hydrogen. In some embodiments, R⁹ and R¹⁰ are each H. In some embodiments, R⁹ is H and R¹⁰ is —C₁-C₄alkylene-(optionally substituted phenyl) or —C₁-C₄alkylene-(optionally substituted heteroaryl). In some embodiments, R⁹ is H and R¹⁰ is —CH₂-(optionally substituted phenyl). In some embodiments, R⁹ is H and R¹⁰ is —CH₂-(optionally substituted heteroaryl). In some embodiments, R¹ is an unsubstituted phenyl. In some embodiments, R¹ is a substituted phenyl. In some embodiments, R¹ is selected from:

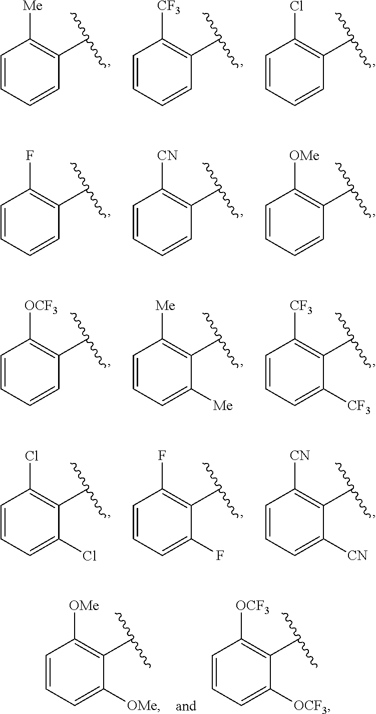

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

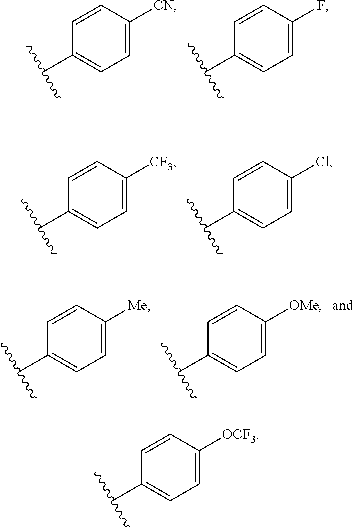

In some embodiments, R² is selected from:

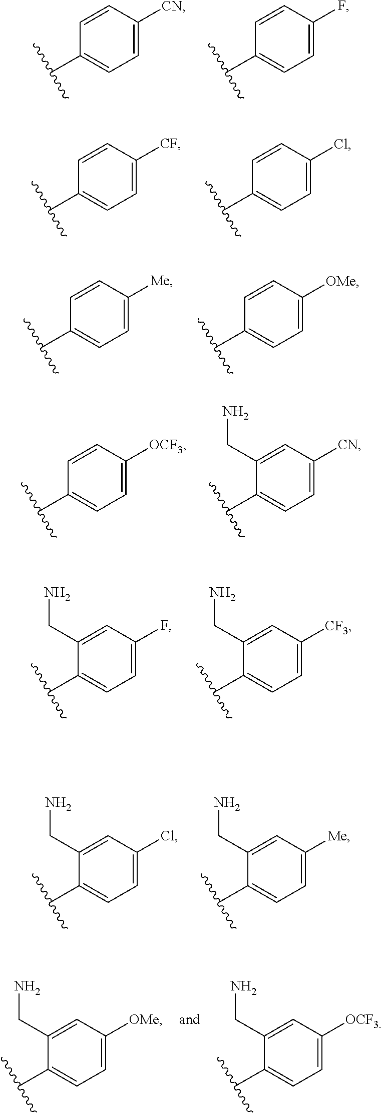

In some embodiments, R¹ is optionally substituted heterocycloalkyl. In some embodiments, R¹ is selected from:

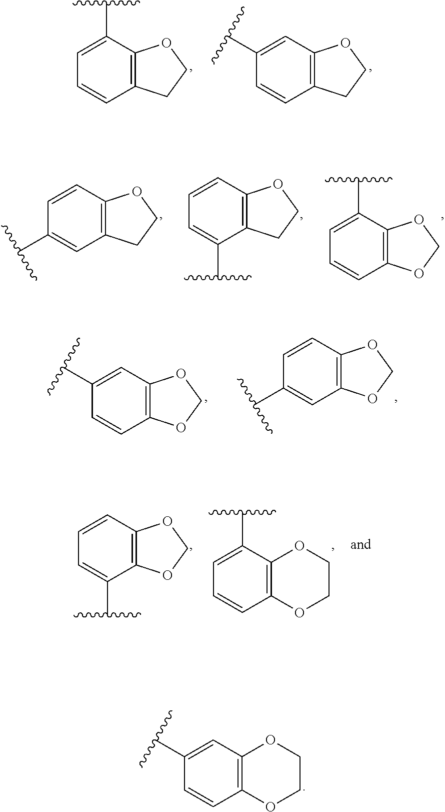

In some embodiments, R² is optionally substituted heterocycloalkyl. In some embodiments, R² is selected from:

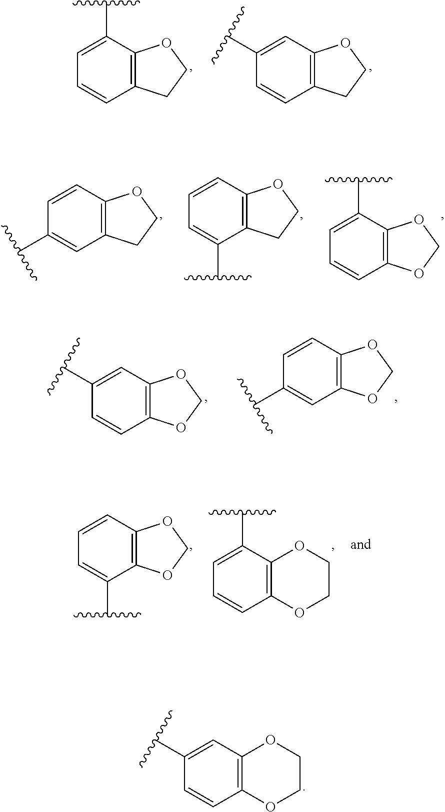

In some embodiments, the compound of Formula (IIIa) is selected from:

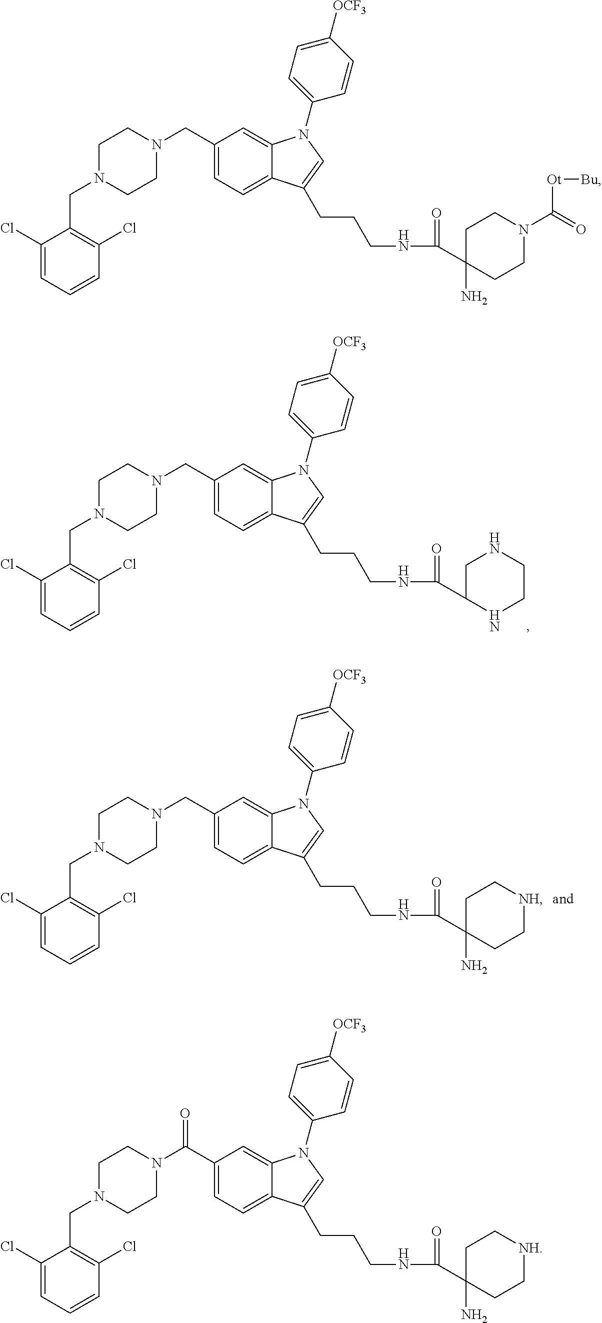

In some embodiments, the compound of Formula (IIIa) is selected from any one of the compounds from the following table:

TABLE 7

| Compound | | |
|---|---|---|
| Ref. | Compound | Chemical Name |
| C73 | 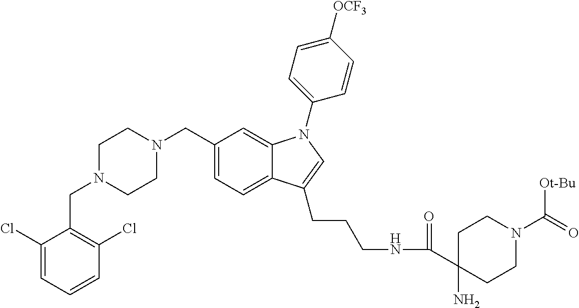 [SMILES: CC(C)(C)OC(=O)N1CCC(N)(C(=O)NCCCc2cn(-c3ccc(OC(F)(F)F)cc3)c3cc(CN4CCN(Cc5c(Cl)cccc5Cl)CC4)ccc23)CC1] | tert-butyl 4-amino-4-(3-(6- ((4-(2,6- dichlorobenzyl)piperazin-1- yl)methyl)-1-(4- (trifluoromethoxy)phenyl)- 1H-indol-3- yl)propylcarbamoyl)piperidine- 1-carboxamide |
| C74 | 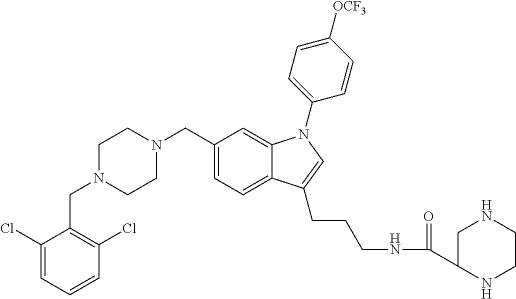 [SMILES: O=C(NCCCc1cn(-c2ccc(OC(F)(F)F)cc2)c2cc(CN3CCN(Cc4c(Cl)cccc4Cl)CC3)ccc12)C1CNCCN1] | N-(3-(6-((4-(2,6- dichlorobenzyl)piperazin-1- yl)methyl)-1-(4- (trifluoromethoxy)phenyl)- 1H-indol-3- yl)propyl)piperazine-2- carboxamide |
| C75 | 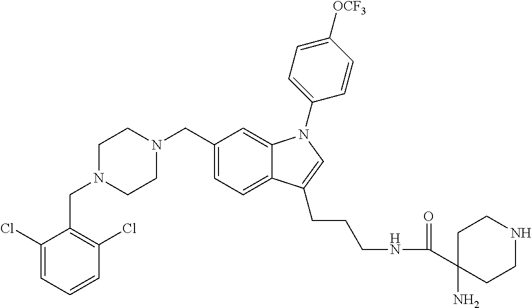 [SMILES: NC1(C(=O)NCCCc2cn(-c3ccc(OC(F)(F)F)cc3)c3cc(CN4CCN(Cc5c(Cl)cccc5Cl)CC4)ccc23)CCNCC1] | 4-amino-N-(3-(6-((4-(2,6- dichlorobenzyl)piperazin-1- yl)methyl)-1-(4- (trifluoromethoxy)phenyl)- 1H-indol-3- yl)propyl)piperidine-4- carboxamide |
| C80 | 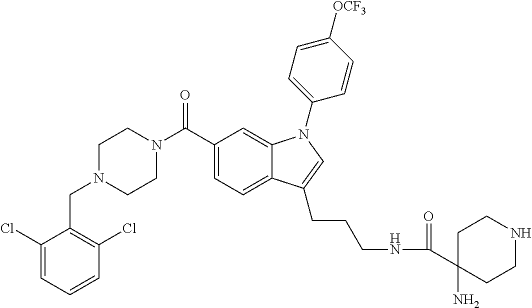 [SMILES: NC1(C(=O)NCCCc2cn(-c3ccc(OC(F)(F)F)cc3)c3cc(C(=O)N4CCN(Cc5c(Cl)cccc5Cl)CC4)ccc23)CCNCC1] | 4-amino-N-(3-(6-(4-(2,6- dichlorobenzyl)piperazine- 1-carbonyl)-1-(4- (trifluoromethoxy)phenyl)- 1H-indol-3- yl)propyl)piperidine-4- carboxamide |

In another aspect provided herein is a compound of Formula (IVa), or a pharmaceutically acceptable salt, or solvate thereof:

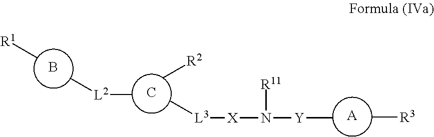

wherein,

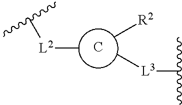

is a bicyclic heteroaryl that is selected from the following structures:

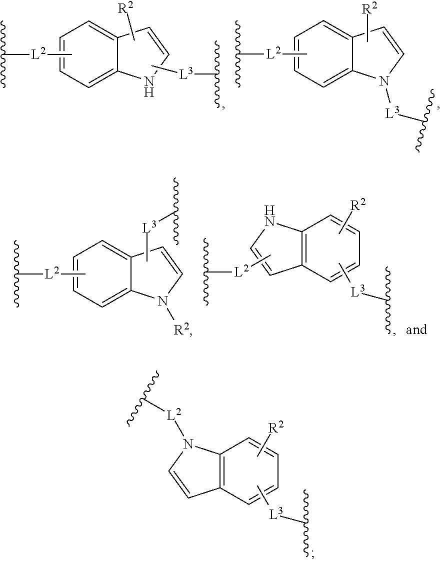

L² is an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N;
wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N;
wherein if Ring A is substituted, then Ring A is substituted with at least one R^{A};
each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring,
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl).

In some embodiments,

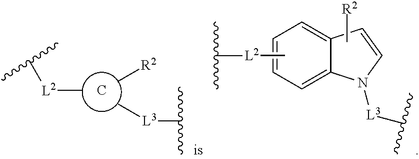

In some embodiments,

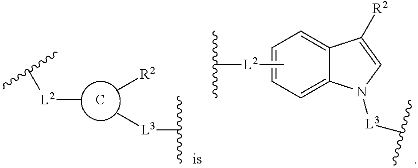

In some embodiments, the compound has the following structure of Formula (IVb), or a pharmaceutically acceptable salt, or solvate thereof:

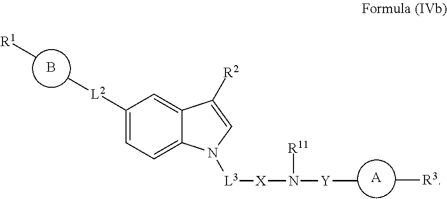

In some embodiments, the compound has the following structure of Formula (IVc), or a pharmaceutically acceptable salt, or solvate thereof:

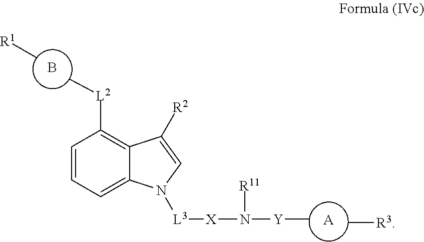

In some embodiments,

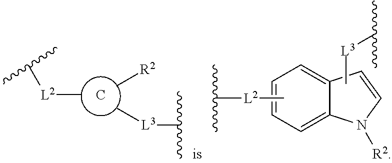

In some embodiments,

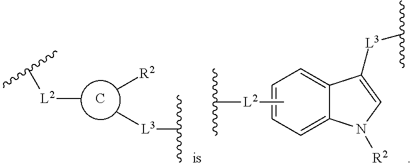

In some embodiments, the compound has the following structure of Formula (IVd), or a pharmaceutically acceptable salt, or solvate thereof:

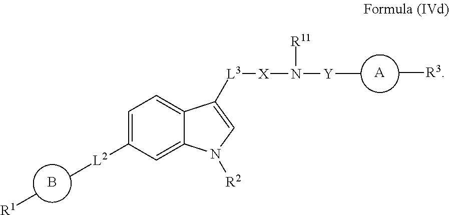

In some embodiments, the compound has the following structure of Formula (IVe), or a pharmaceutically acceptable salt, or solvate thereof:

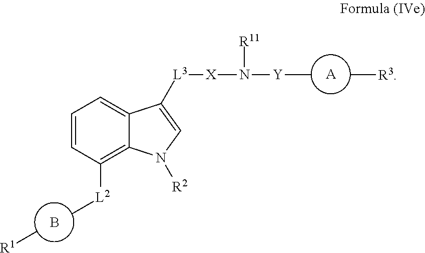

In some embodiments,

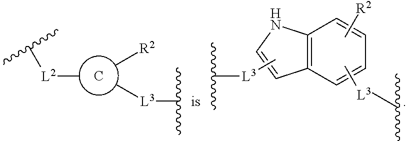

In some embodiments,

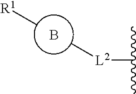

is selected from the following:

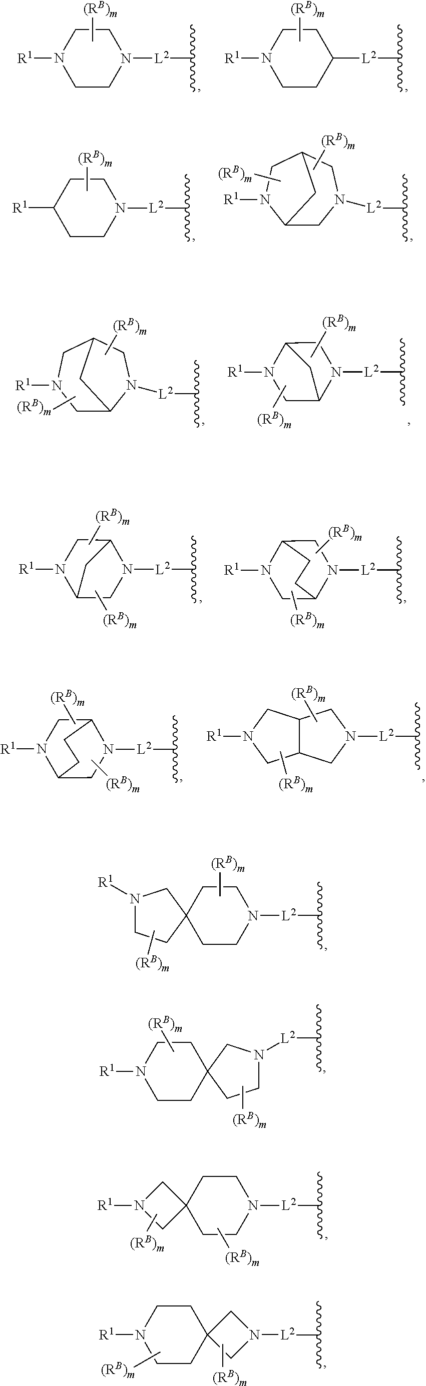

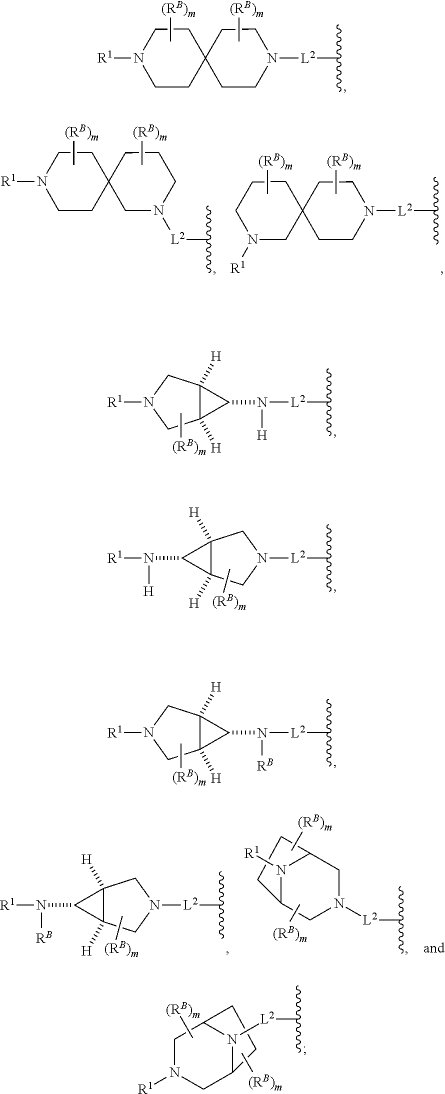

and each m is independently, 0, 1, 2, 3, or 4.

In some embodiments,

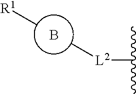

is selected from the following:

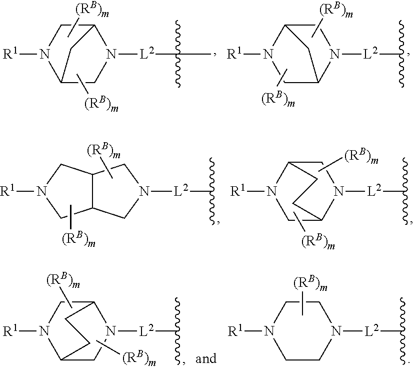

In some embodiments,

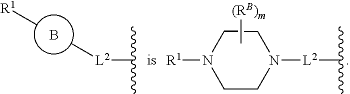

In some embodiments,

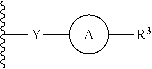

is selected from the following:

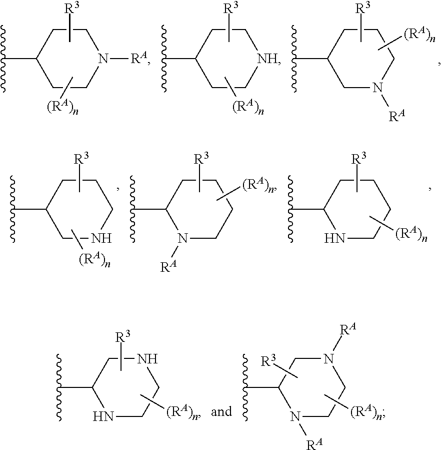

and each n is independently 0, 1, 2, 3, or 4.

In some embodiments,

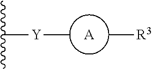

is selected from the following:

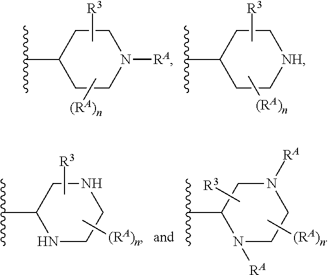

In some embodiments,

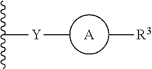

is selected from the following:

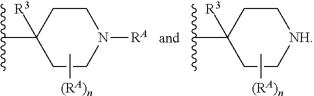

In some embodiments, L² is —CH₂—, C(═O), O, S, S(═O), S(═O)₂, or NR⁴. In some embodiments, L² is —CH₂—. In some embodiments, L³ is absent, —CH₂—, —CH₂—CH₂—, or —CH₂—CH₂—CH₂—. In some embodiments, L³ is —CH₂—CH₂—.

In some embodiments, L³ is:

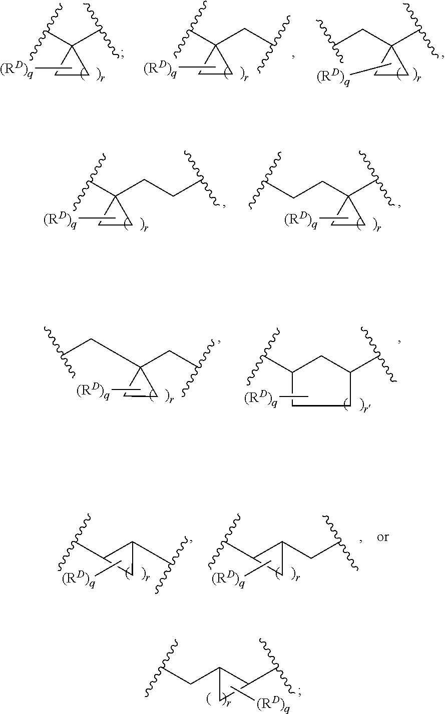

each q is independently 0, 1, 2, 3, or 4;

r is 1, 2, 3, or 4; and

r′ is 1 or 2.

In some embodiments, X is —CH₂— or C(═O). In some embodiments, X is —CH₂—. In some embodiments, X is:

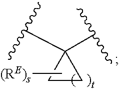

each s is independently 0, 1, 2, 3, or 4; and

t is 1, 2, 3, or 4.

In some embodiments, X is:

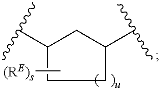

and

each s is independently 0, 1, 2, 3, or 4; and

u is 0, 1, or 2.

In some embodiments, L³-X is —CH₂—CH₂—CH₂—. In some embodiments, Y is —CH₂— or C(═O). In some embodiments, Y is C(═O). In some embodiments, R¹¹ is hydrogen. In some embodiments, R⁹ and R¹⁰ are each H. In some embodiments, R⁹ is H and R¹⁰ is —C₁-C₄alkylene-(optionally substituted phenyl) or —C₁-C₄alkylene-(optionally substituted heteroaryl). In some embodiments, R⁹ is H and R¹⁰ is —CH₂-(optionally substituted phenyl). In some embodiments, R⁹ is H and R¹⁰ is —CH₂-(optionally substituted heteroaryl). In some embodiments, R¹ is an hydrogen, an optionally substituted C₁-C₆alkyl, or an optionally substituted aryl. In some embodiments, R¹ is hydrogen. In some embodiments, R¹ is an unsubstituted C₁-C₆alkyl. In some embodiments, R¹ is a substituted C₁-C₆alkyl. In some embodiments, R¹ is an unsubstituted phenyl. In some embodiments, R¹ is a substituted phenyl. In some embodiments, R¹ is selected from:

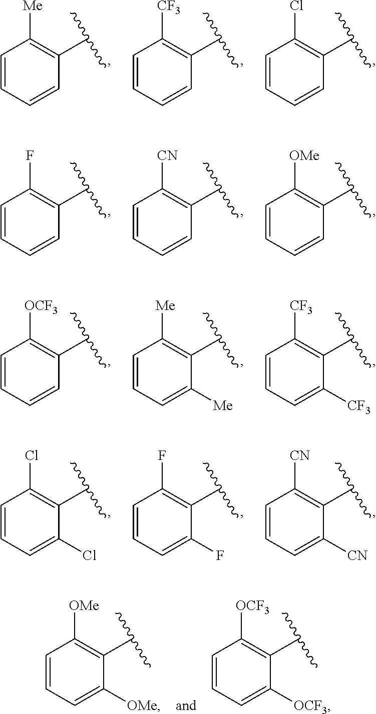

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

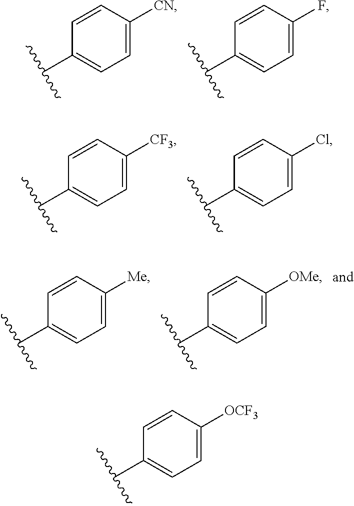

In some embodiments, R² is selected from:

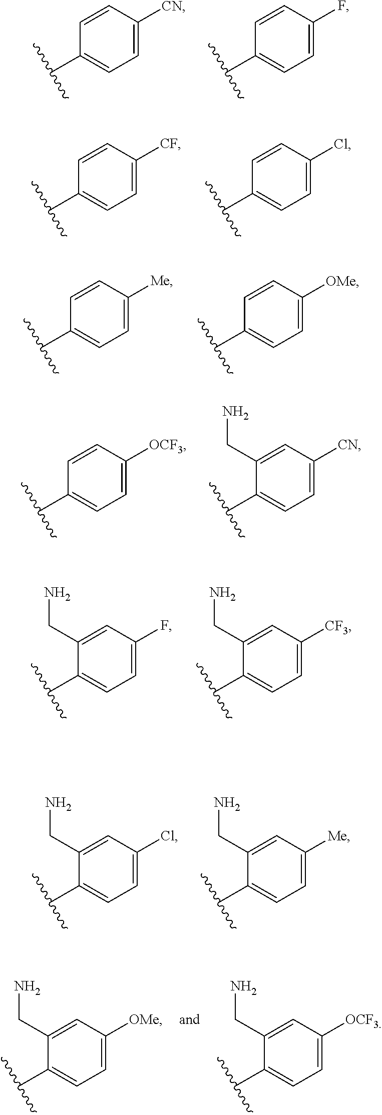

In some embodiments, R¹ is optionally substituted heterocycloalkyl. In some embodiments, R¹ is selected from:

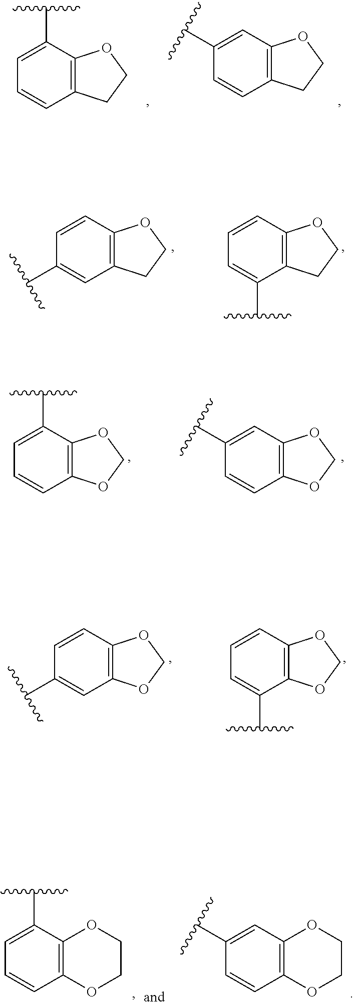

In some embodiments, R² is optionally substituted heterocycloalkyl. In some embodiments, R² is selected from:

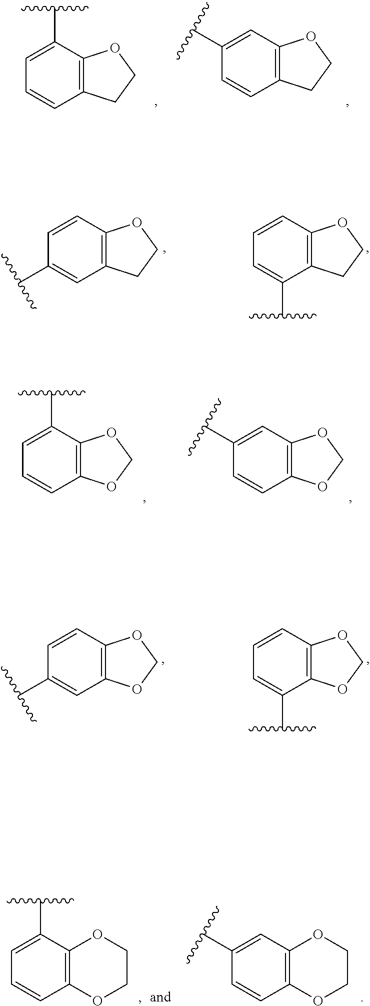

In some embodiments, the compound of Formula (IVa) is selected from:

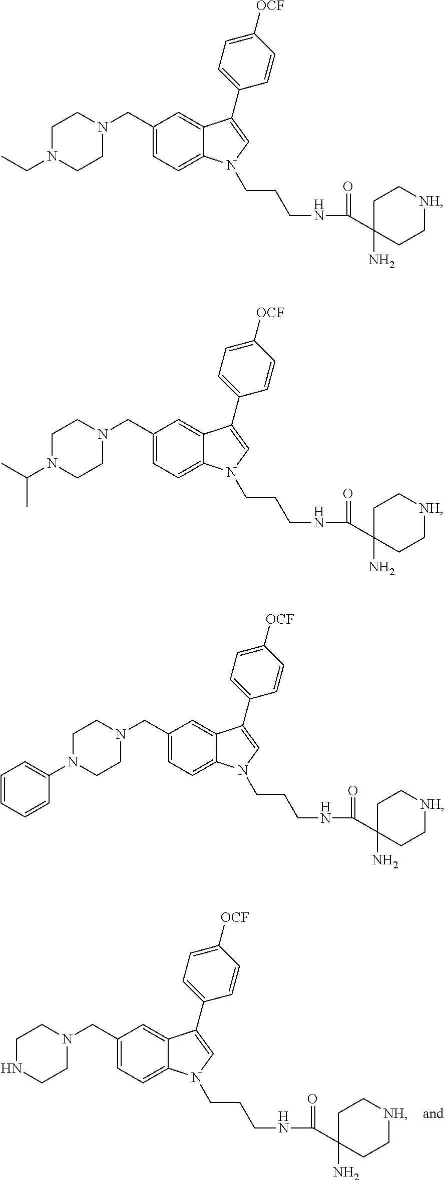

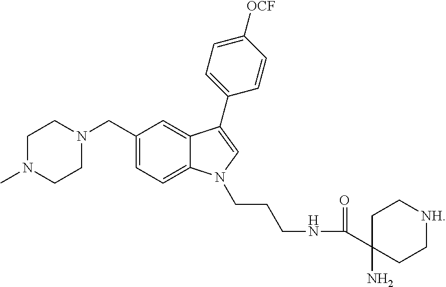

In some embodiments, the compound of Formula (IVa) is selected from any one of the compounds from the following table:

TABLE 8

| Compound | | |
|---|---|---|
| Ref. | Compound | Chemical Name |
| C27 | 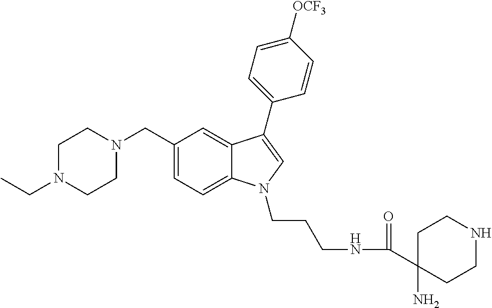 [SMILES: CCN1CCN(Cc2ccc3c(c2)c(-c2ccc(OC(F)(F)F)cc2)cn3CCCNC(=O)C2(N)CCNCC2)CC1] | 4-amino-N-(3-(5-((4- ethylpiperazin-1-yl)methyl)-3- (4-(trifluoromethoxy)phenyl)- 1H-indol-1- yl)propyl)piperidine-4- carboxamide |
| C34 | 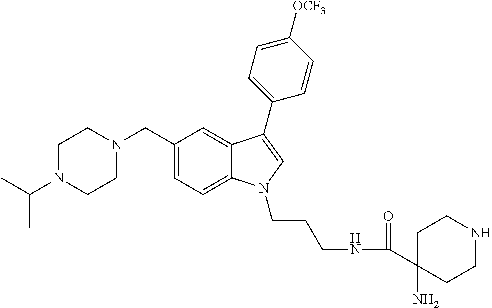 [SMILES: CC(C)N1CCN(Cc2ccc3c(c2)c(-c2ccc(OC(F)(F)F)cc2)cn3CCCNC(=O)C2(N)CCNCC2)CC1] | 4-amino-N-(3-(5-((4- isopropylpiperazin-1- yl)methyl)-3-(4- (trifluoromethoxy)phenyl)-1H- indol-1-yl)propyl)piperidine-4- carboxamide |
| C38 | 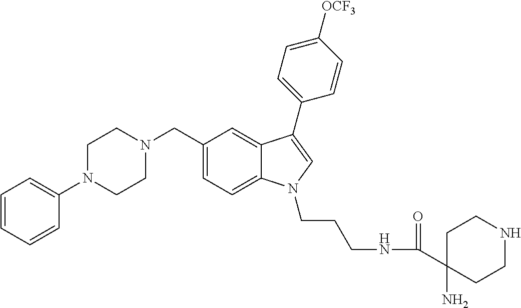 [SMILES: NC1(C(=O)NCCCn2cc(-c3ccc(OC(F)(F)F)cc3)c3cc(CN4CCN(c5ccccc5)CC4)ccc32)CCNCC1] | 4-amino-N-(3-(5-((4- phenylpiperazin-1-yl)methyl)- 3-(4- (trifluoromethoxy)phenyl)-1H- indol-1-yl)propyl)piperidine-4- carboxamide |
| C48 | 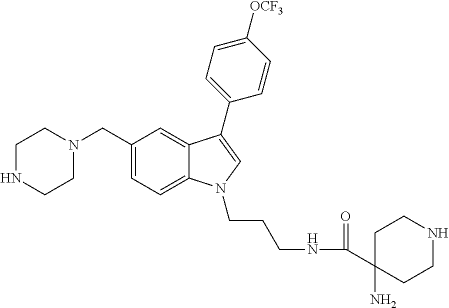 [SMILES: NC1(C(=O)NCCCn2cc(-c3ccc(OC(F)(F)F)cc3)c3cc(CN4CCNCC4)ccc32)CCNCC1] | 4-amino-N-(3-(5-(piperazin-1- ylmethyl)-3-(4- (trifluoromethoxy)phenyl)-1H- indol-1-yl)propyl)piperidine-4- carboxamide |
| C57 | 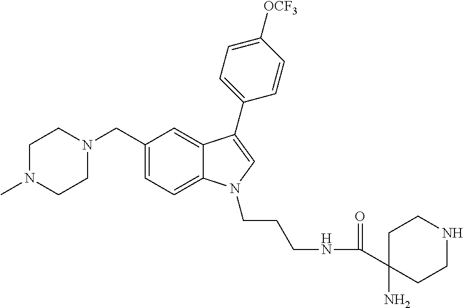 [SMILES: CN1CCN(Cc2ccc3c(c2)c(-c2ccc(OC(F)(F)F)cc2)cn3CCCNC(=O)C2(N)CCNCC2)CC1] | 4-amino-N-(3-(5-((4- methylpiperazin-1-yl)methyl)- 3-(4- (trifluoromethoxy)phenyl)-1H- indol-1-yl)piperidin-4- carboxamide |

Also provided herein in another aspect is a compound of Formula (Va), or a pharmaceutically acceptable salt, or solvate thereof:

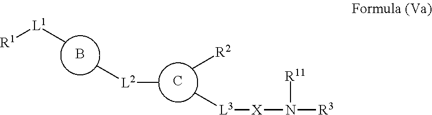

wherein,

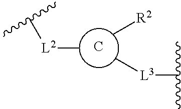

is a bicyclic heteroaryl that is selected from the following structures:

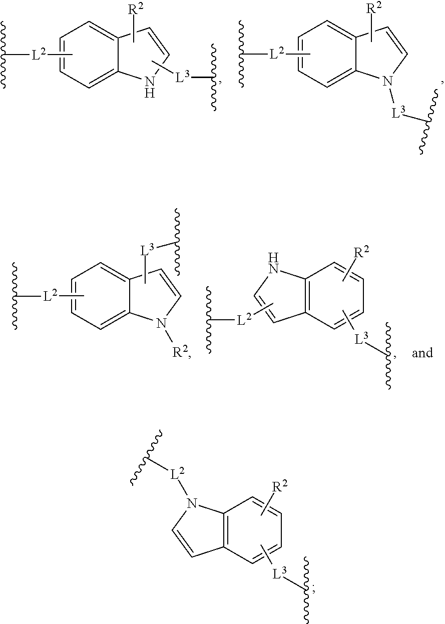

L¹ and L² are each independently absent, an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N with the proviso that Ring B is not:

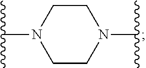 [SMILES: *N1CCN(*)CC1]

wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted phenylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
R³ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, —C₁-C₄alkylene-(optionally substituted heteroaryl), —CH₂C(═O)R¹⁵, —C(═O)R¹⁵, or —CO₂R¹⁶;
R⁴ is hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹⁵ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁶ is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl.

In some embodiments,

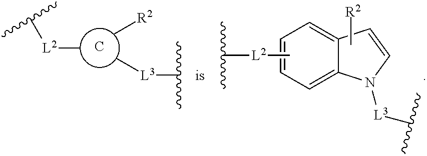

In some embodiments,

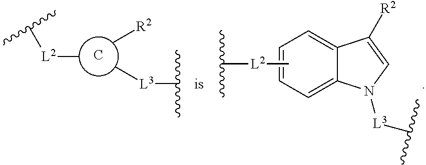

In some embodiments, the compound has the structure of Formula (Vb), or a pharmaceutically acceptable salt, or solvate thereof:

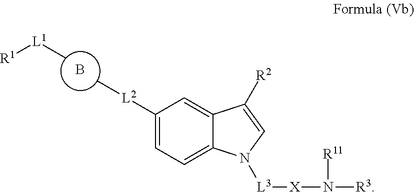

In some embodiments, the compound has the structure of Formula (Vc), or a pharmaceutically acceptable salt, or solvate thereof:

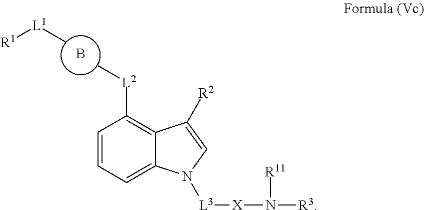

In some embodiments,

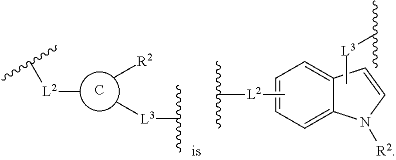

In some embodiments,

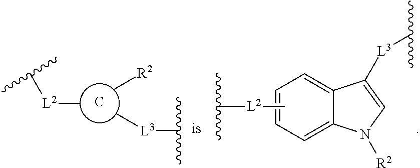

In some embodiments, the compound has the structure of Formula (Vd), or a pharmaceutically acceptable salt, or solvate thereof:

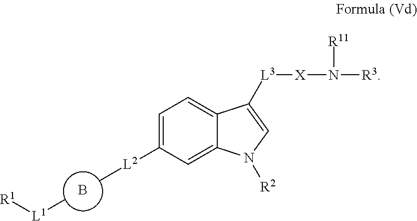

In some embodiments, the compound has the structure of Formula (Ve), or a pharmaceutically acceptable salt, or solvate thereof:

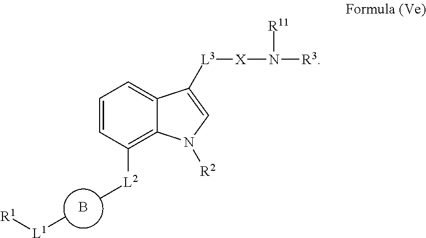

In some embodiments,

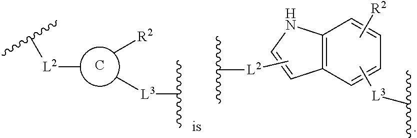

In some embodiments,

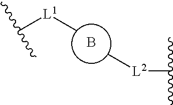

is selected from the following:

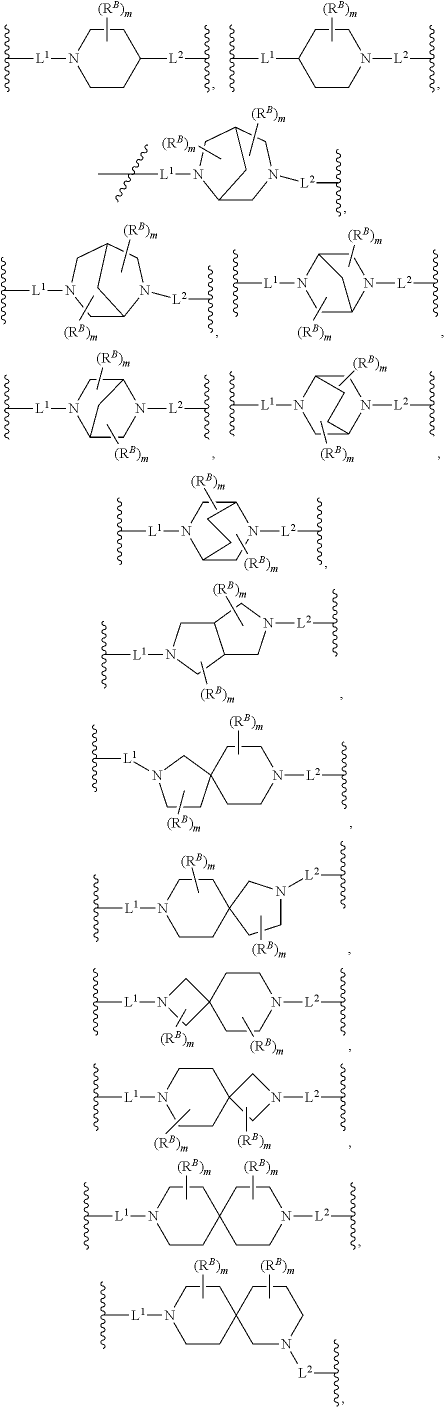

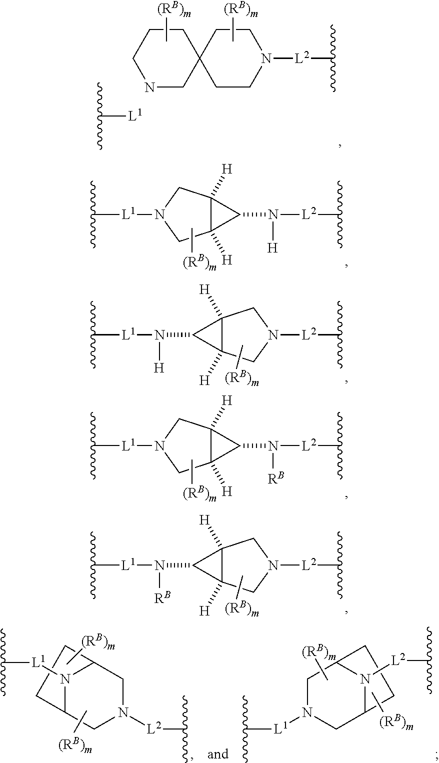

and each m is independently 0, 1, 2, 3, or 4.

In some embodiments,

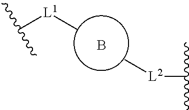

is selected from the following:

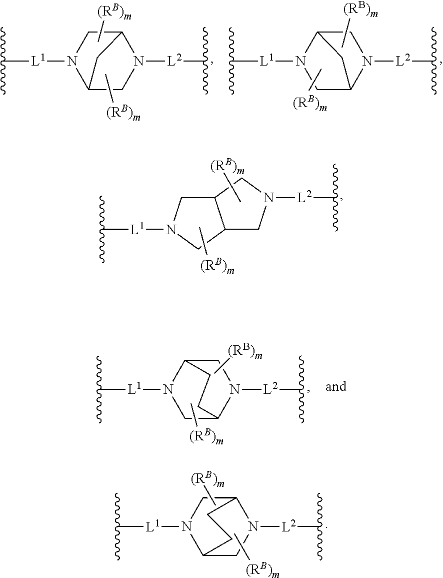

In some embodiments,

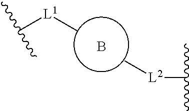

is selected from the following:

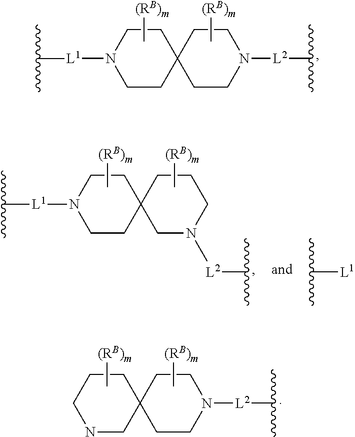

In some embodiments,

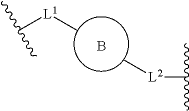

is selected from the following:

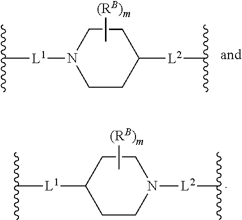

In some embodiments,

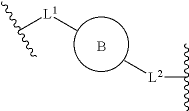

is selected from the following:

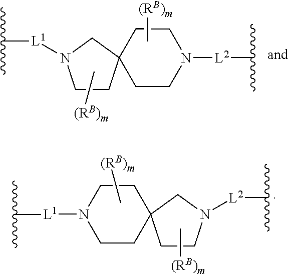

In some embodiments,

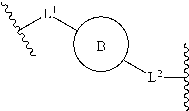

is selected from the following:

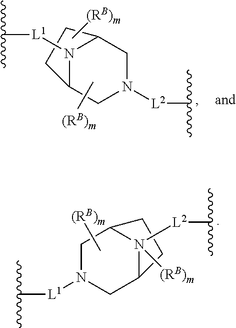

In some embodiments, L¹ is —CH₂—, C(═O), O, S, S(═O), S(═O)₂, or NR⁴. In some embodiments, L¹ is —CH₂—. In some embodiments, L² is —CH₂—, C(═O), O, S, S(═O), S(═O)₂, or NR⁴. In some embodiments, L² is —CH₂—. In some embodiments, L³ is absent, —CH₂—, —CH₂—CH₂—, or —CH₂—CH₂—CH₂—. In some embodiments, L³ is —CH₂—CH₂—.

In some embodiments, L³ is:

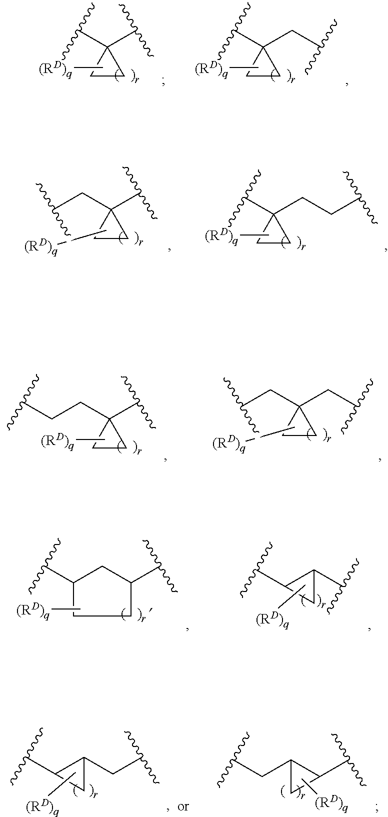

each q is independently 0, 1, 2, 3, or 4;

r is 1, 2, 3, or 4; and

r′ is 1 or 2.

In some embodiments, X is —CH₂— or C(═O). In some embodiments, X is —CH₂—.

In some embodiments, X is:

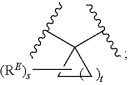

each s is independently 0, 1, 2, 3, or 4; and

t is 1, 2, 3, or 4.

In some embodiments, X is:

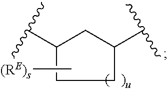

and

each s is independently 0, 1, 2, 3, or 4; and

u is 0, 1, or 2.

In some embodiments, L³-X is —CH₂—CH₂—CH₂—. In some embodiments, R¹¹ and R³ are each independently hydrogen, optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹¹ and R³ are each hydrogen. In some embodiments, R¹¹ and R³ are each optionally substituted C₁-C₆alkyl. In some embodiments, R¹¹ and R³ are each optionally substituted C₁-C₆heteroalkyl. In some embodiments, R¹⁵ and R¹⁶ are each independently optionally substituted C₁-C₆alkyl or optionally substituted C₁-C₆heteroalkyl. In some embodiments, R¹¹ is hydrogen and R³ is optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹ is an unsubstituted phenyl. In some embodiments, R¹ is a substituted phenyl. In some embodiments, R¹ is selected from:

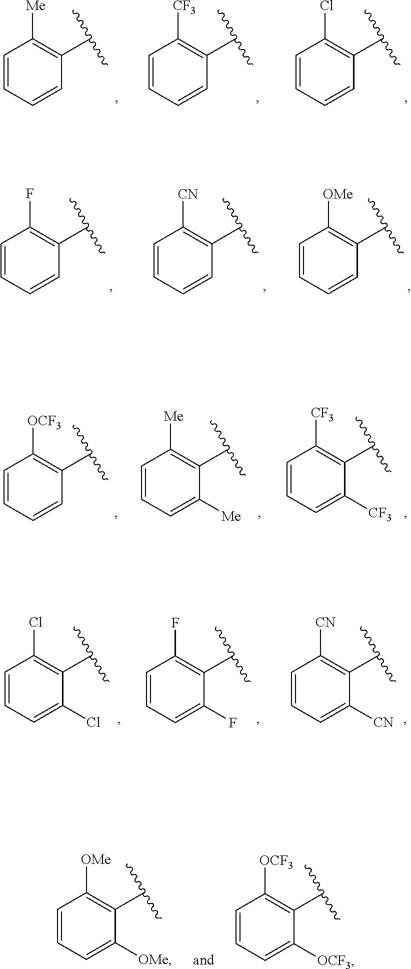

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

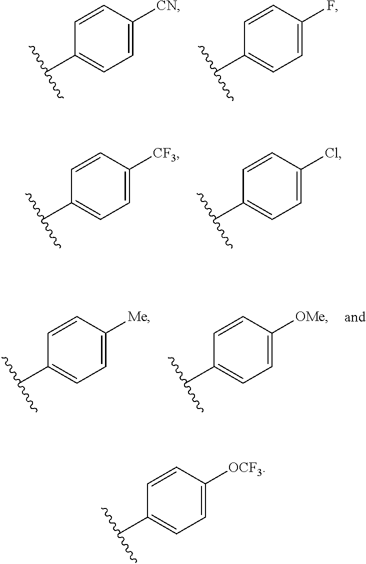

In some embodiments, R² is selected from:

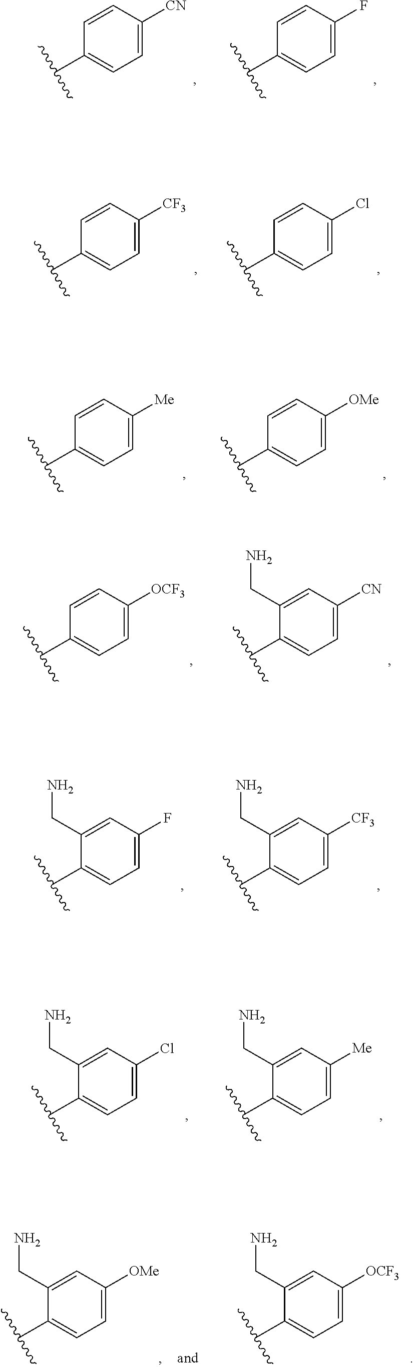

In some embodiments, R¹ is optionally substituted heterocycloalkyl. In some embodiments, R¹ is selected from:

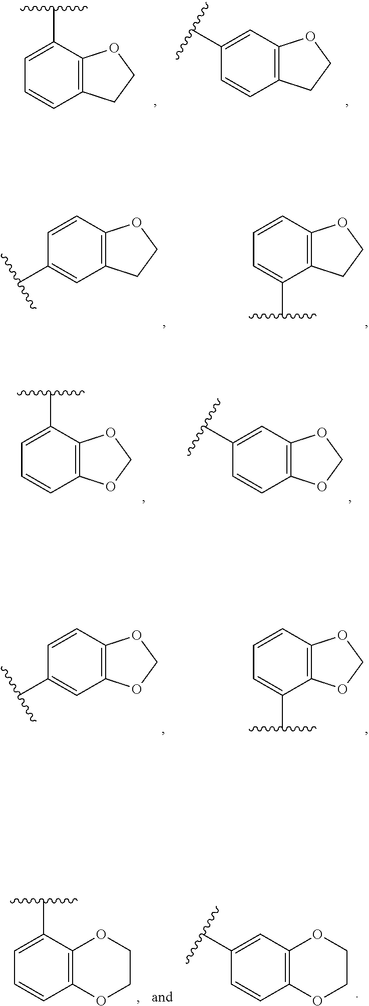

In some embodiments, R² is optionally substituted heterocycloalkyl. In some embodiments, R² is selected from:

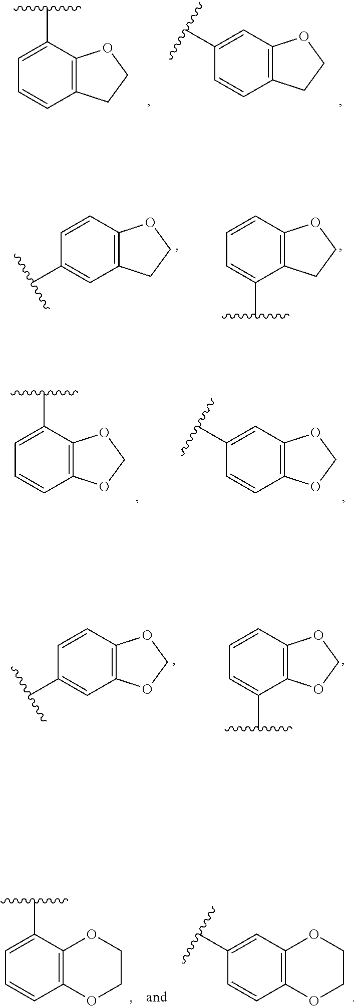

In some embodiments, the compound has the following structure of Formula (Vf), or a pharmaceutically acceptable salt, or solvate thereof:

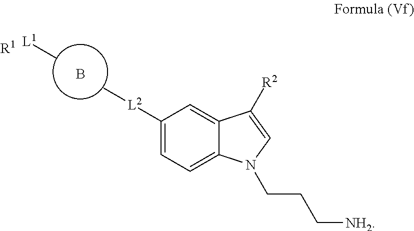

In some embodiments, the compound has the following structure of Formula (Vg), or a pharmaceutically acceptable salt, or solvate thereof:

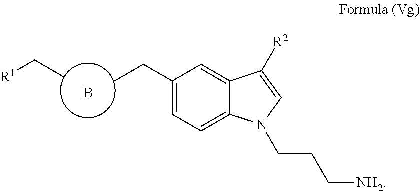

wherein R¹ and R² are each independently optionally substituted aryl; and

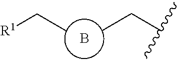

is selected from

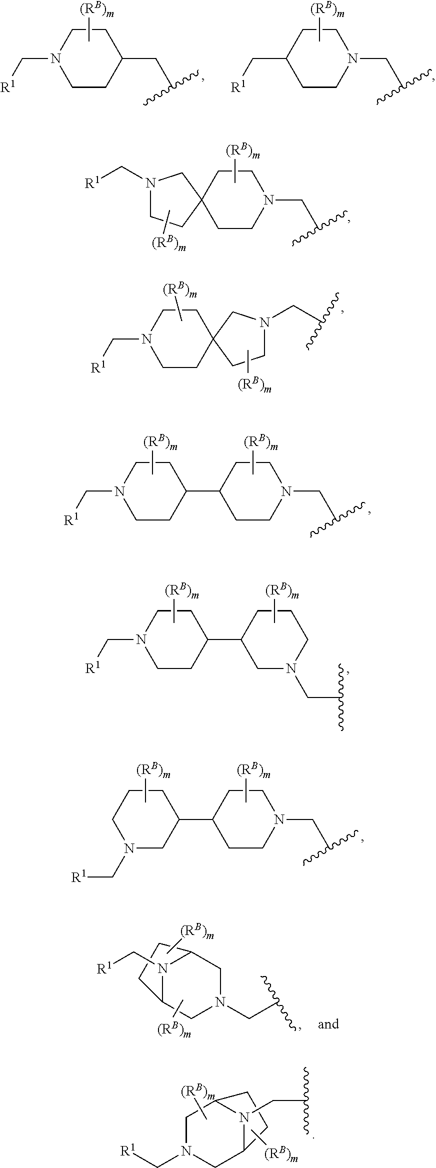

In some embodiments,

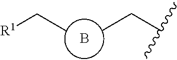

is selected from

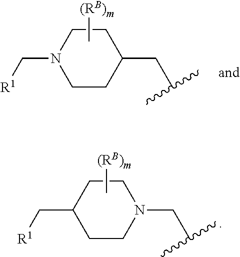

In some embodiments,

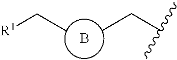

is selected from

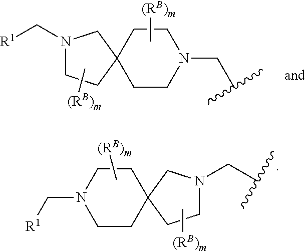

In some embodiments,

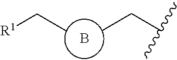

is selected from

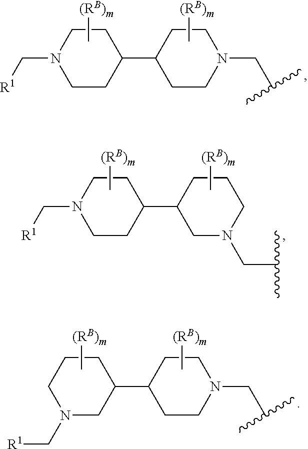

In some embodiments,

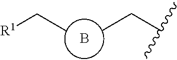

is selected from

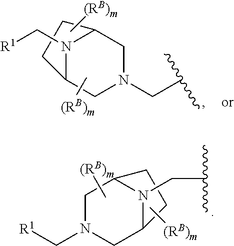

In some embodiments, the compound of Formula (Va) is selected from:

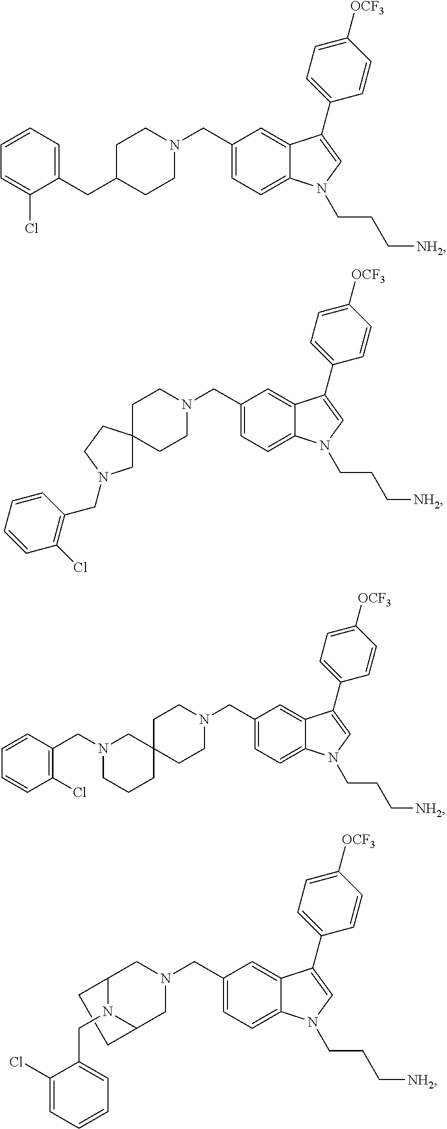

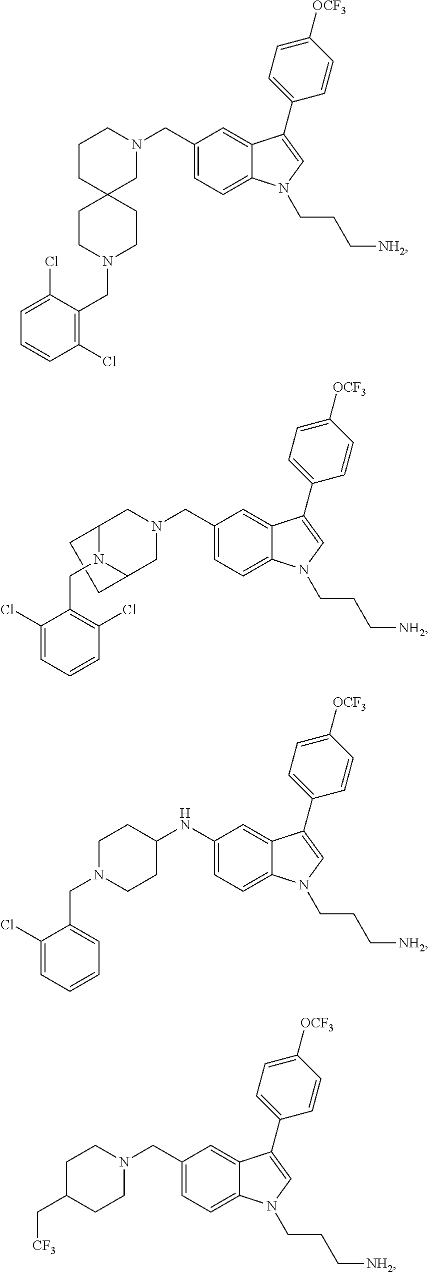 [SMILES: NCCCn1cc(-c2ccc(OC(F)(F)F)cc2)c2cc(CN3CCCC4(CCN(Cc5c(Cl)cccc5Cl)CC4)C3)ccc21 ; NCCCn1cc(-c2ccc(OC(F)(F)F)cc2)c2cc(CN3CC4CCC(C3)N4Cc3c(Cl)cccc3Cl)ccc21 ; NCCCn1cc(-c2ccc(OC(F)(F)F)cc2)c2cc(NC3CCN(Cc4ccccc4Cl)CC3)ccc21 ; NCCCn1cc(-c2ccc(OC(F)(F)F)cc2)c2cc(CN3CCC(CC(F)(F)F)CC3)ccc21]

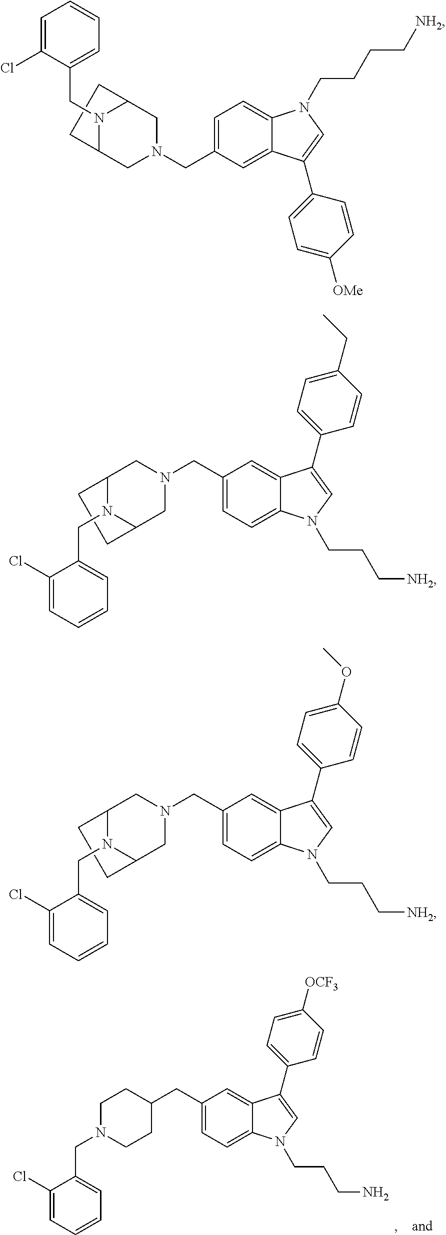

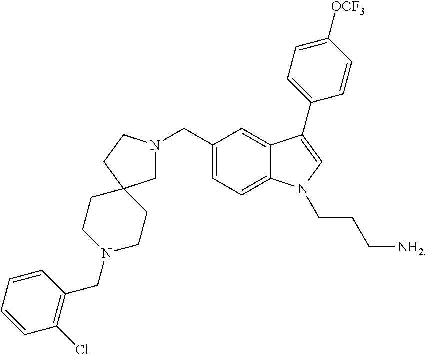 [SMILES: NCCCn1cc(-c2ccc(OC(F)(F)F)cc2)c2cc(CN3CCC4(CCN(Cc5ccccc5Cl)CC4)C3)ccc21]

In some embodiments, the compound of Formula (Va) is selected from:

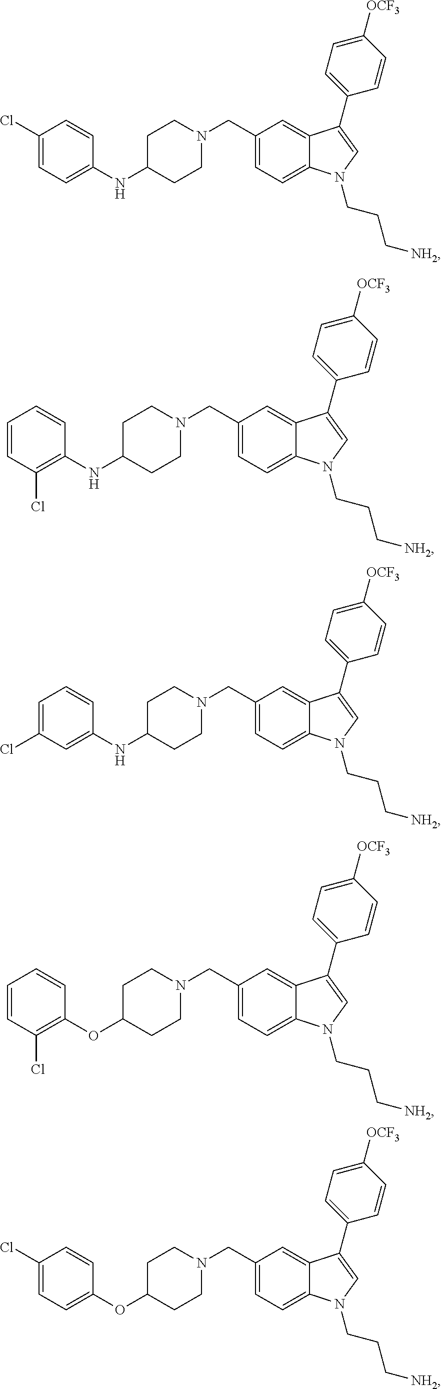

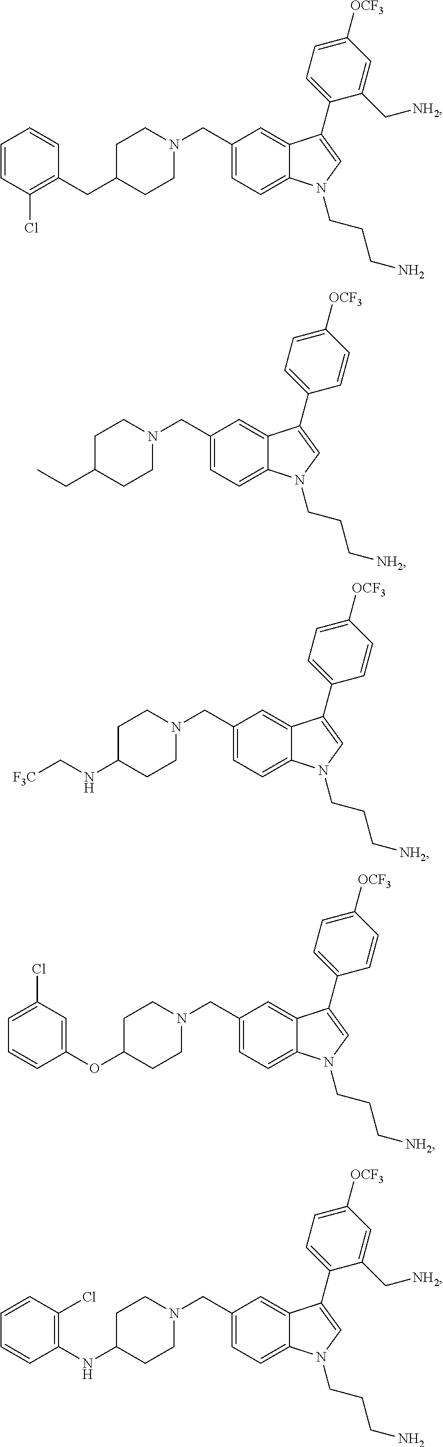

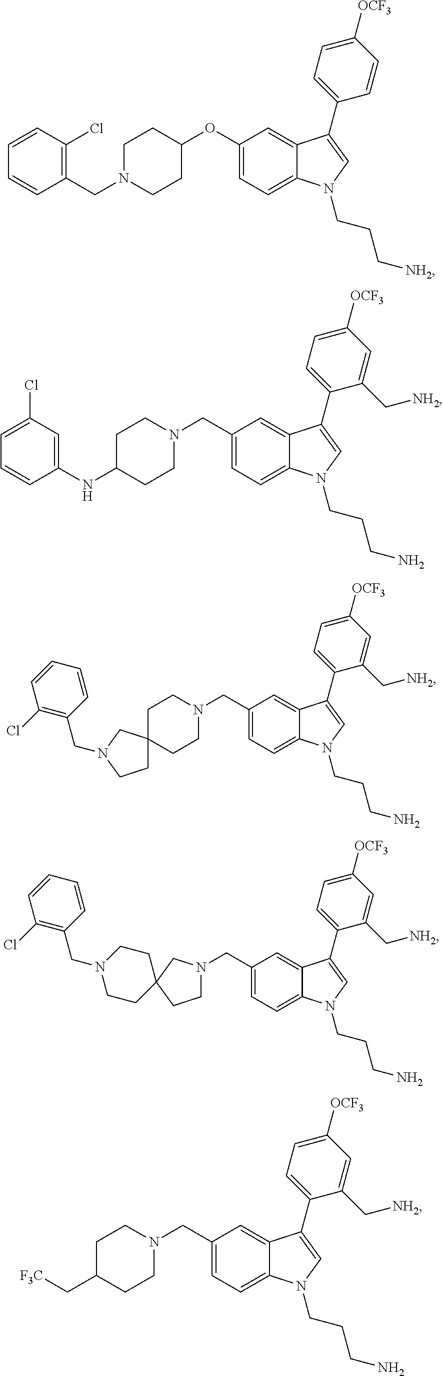

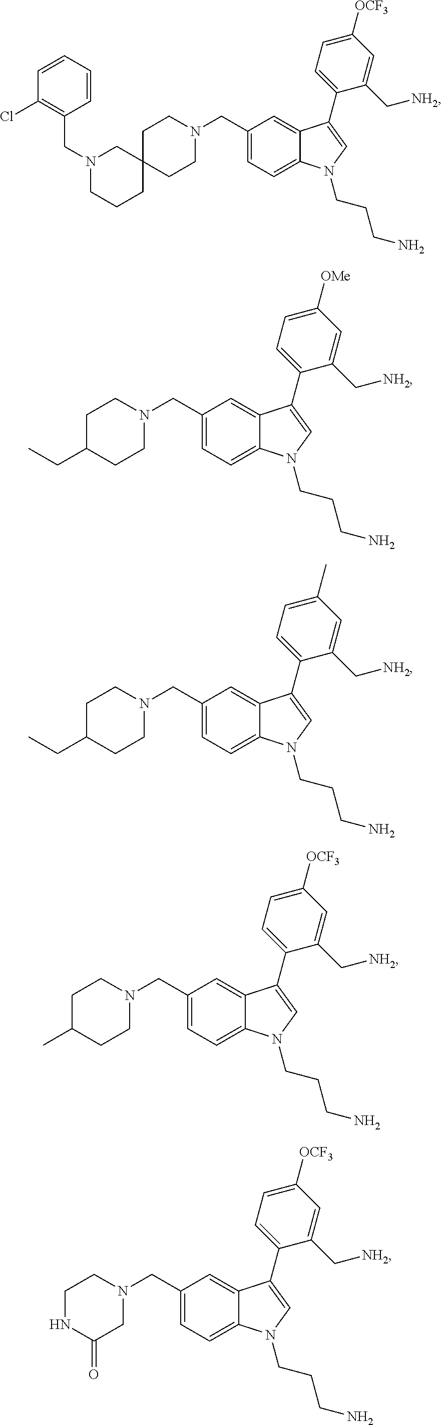

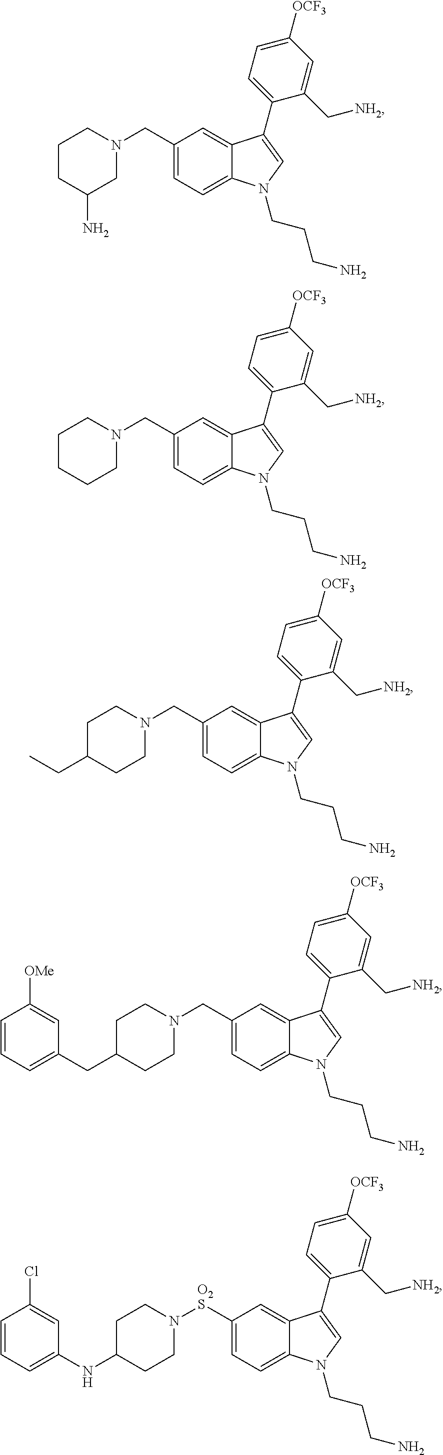

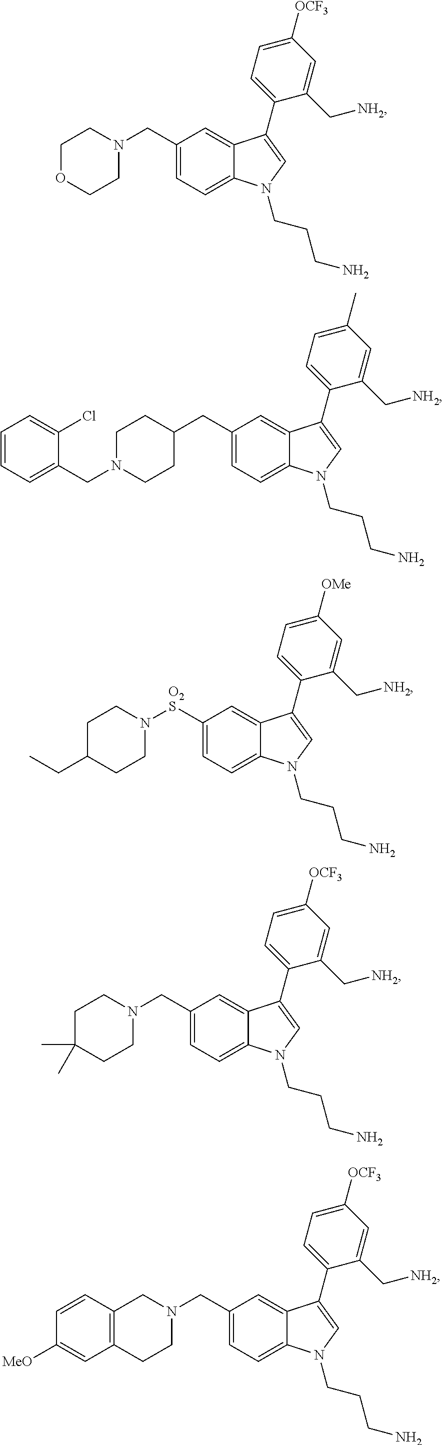

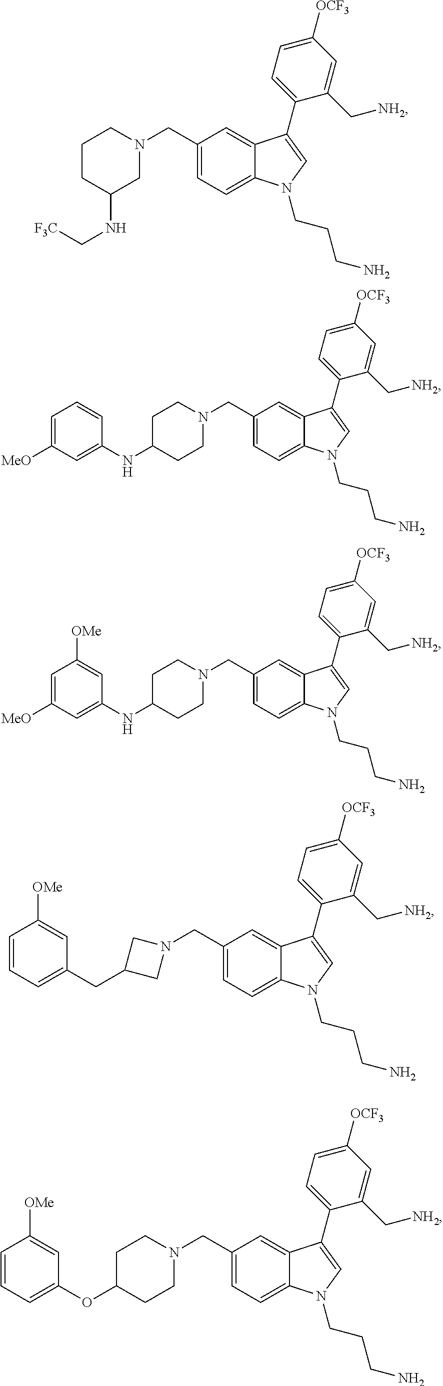

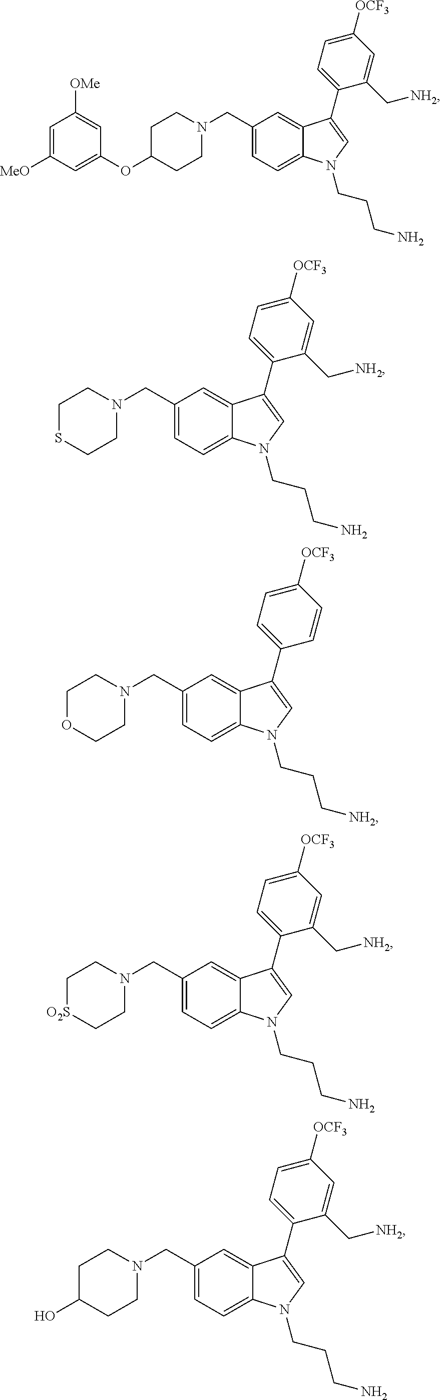

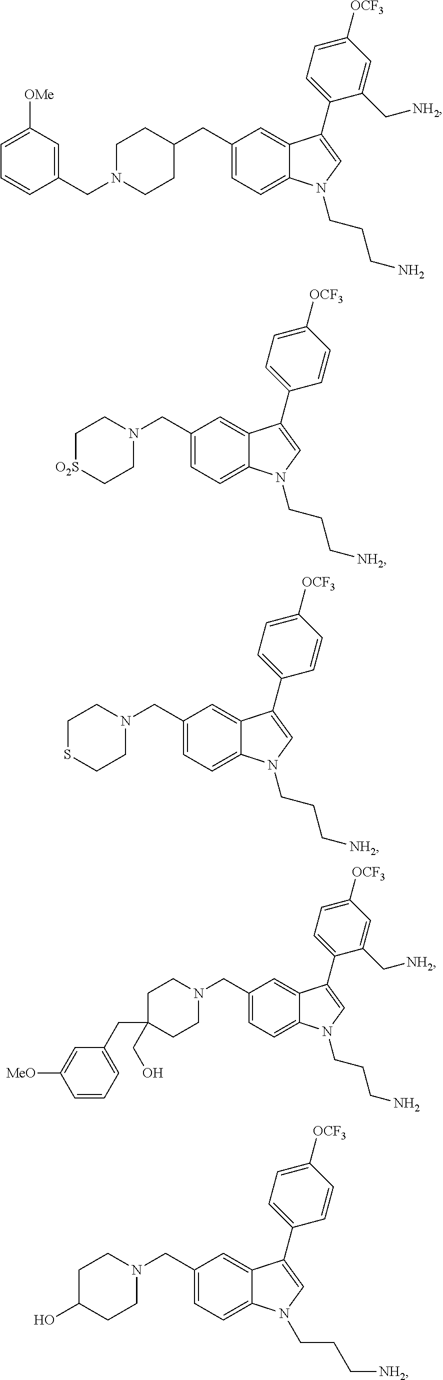

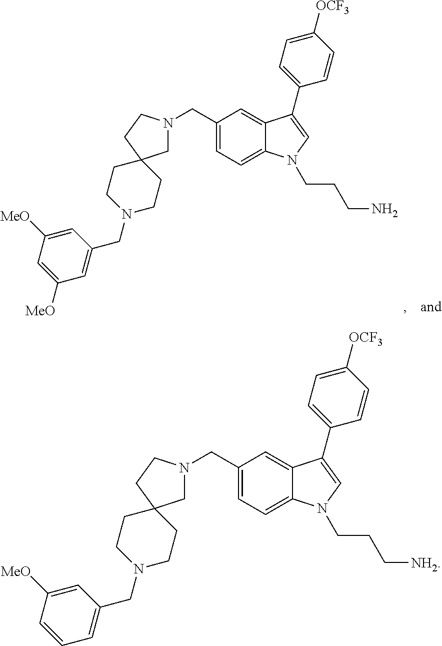 [SMILES: COc1cc(CN2CCC3(CC2)CCN(Cc2ccc4c(c2)c(-c2ccc(OC(F)(F)F)cc2)cn4CCCN)C3)cc(OC)c1 ; COc1cccc(CN2CCC3(CC2)CCN(Cc2ccc4c(c2)c(-c2ccc(OC(F)(F)F)cc2)cn4CCCN)C3)c1]

In some embodiments, the compound of Formula (Va) is selected from any one of the compounds from the following table:

TABLE 9A

| Compound | | |
|---|---|---|
| Ref. | Compound | Chemical Name |
| C3 | 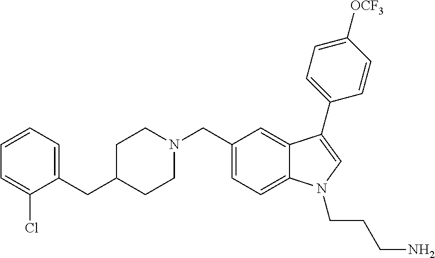 [SMILES: NCCCn1cc(-c2ccc(OC(F)(F)F)cc2)c2cc(CN3CCC(Cc4ccccc4Cl)CC3)ccc21] | 3-(5-((4-(2- chlorobenzyl)piperidin-1- yl)methyl)-3-(4- (trifluoromethoxy)phenyl)- 1H-indol-1-yl)propan-1- amine |
| C5 | 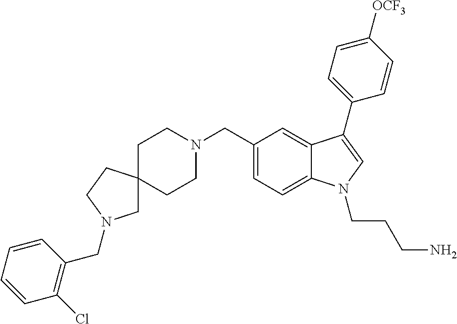 [SMILES: NCCCn1cc(-c2ccc(OC(F)(F)F)cc2)c2cc(CN3CCC4(CC3)CCN(Cc3ccccc3Cl)C4)ccc21] | 3-(5-((2-(2-chlorobenzyl)- 2,8-diazaspiro[4.5]decan-8- yl)methyl)-3-(4- (trifluoromethoxy)phenyl)- 1H-indol-1-yl)propan-1- amine |
| C6 | 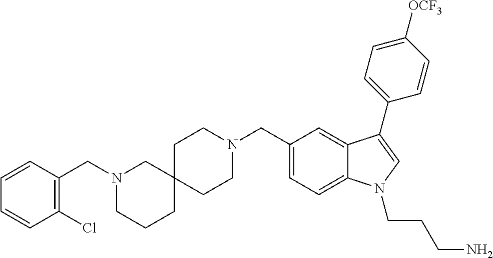 [SMILES: NCCCn1cc(-c2ccc(OC(F)(F)F)cc2)c2cc(CN3CCC4(CCCN(Cc5ccccc5Cl)C4)CC3)ccc21] | 3-(5-((2-(2-chlorobenzyl)- 2,9-diazaspiro[5.5]undecan- 9-yl)methyl)-3-(4- (trifluoromethoxy)phenyl)- 1H-indol-1-yl)propan-1- amine |
| C17 | 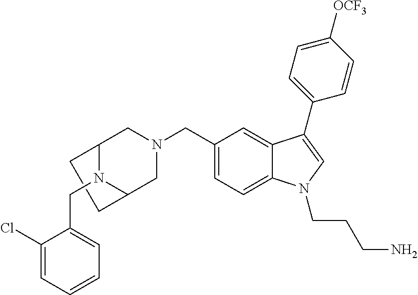 [SMILES: NCCCn1cc(-c2ccc(OC(F)(F)F)cc2)c2cc(CN3CC4CCC(C3)N4Cc3ccccc3Cl)ccc21] | 3-(5-((8-(2-chlorobenzyl)- 3,8-diazabicyclo[3.2.1]octan- 3-yl)methyl)-3-(4- (trifluoromethoxy)phenyl)- 1H-indol-1-yl)propan-1- amine |
| C31 | 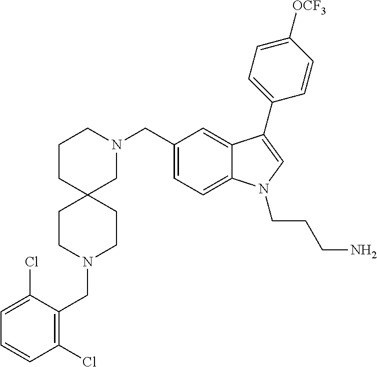 [SMILES: NCCCn1cc(-c2ccc(OC(F)(F)F)cc2)c2cc(CN3CCCC4(CCN(Cc5c(Cl)cccc5Cl)CC4)C3)ccc21] | 3-(5-((9-(2,6- dichlorobenzyl)-2,9- diazaspiro[5.5]undecan-2- yl)methyl)-3-(4- (trifluoromethoxy)phenyl)- 1H-indol-1-yl)propan-1- amine |
| C32 | 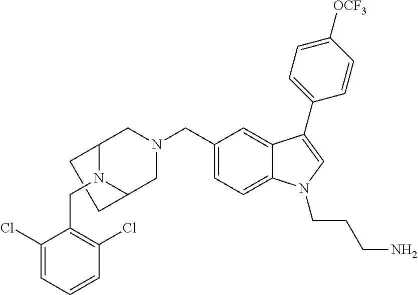 [SMILES: NCCCn1cc(-c2ccc(OC(F)(F)F)cc2)c2cc(CN3CC4CCC(C3)N4Cc3c(Cl)cccc3Cl)ccc21] | 3-(5-((8-(2,6- dichlorobenzyl)-3,8- diazabicyclo[3.2.1]octan-3- yl)methyl)-3-(4- (trifluoromethoxy)phenyl)- 1H-indol-1-yl)propan-1- amine |
| C129 | 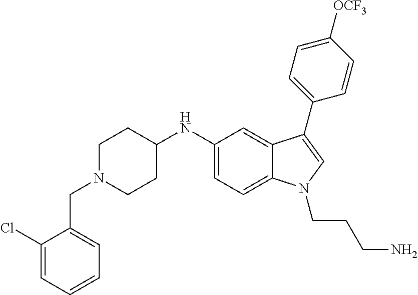 [SMILES: NCCCn1cc(-c2ccc(OC(F)(F)F)cc2)c2cc(NC3CCN(Cc4ccccc4Cl)CC3)ccc21] | 1-(3-aminopropyl)-N-(1-(2- chlorobenzyl)piperidin-4-yl)- 3-(4- (trifluoromethoxy)phenyl)- 1H-indol-5-amine |
| C130 | 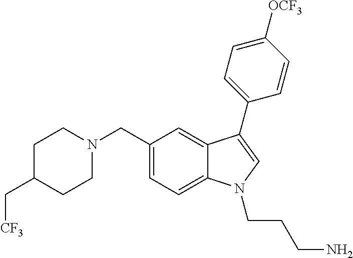 [SMILES: NCCCn1cc(-c2ccc(OC(F)(F)F)cc2)c2cc(CN3CCC(CC(F)(F)F)CC3)ccc21] | 3-(5-((4-(2,2,2- trifluoroethyl)piperidin-1- yl)methyl)-3-(4- (trifluoromethoxy)phenyl)- 1H-indol-1-yl)propan-1- amine |
| C133 | 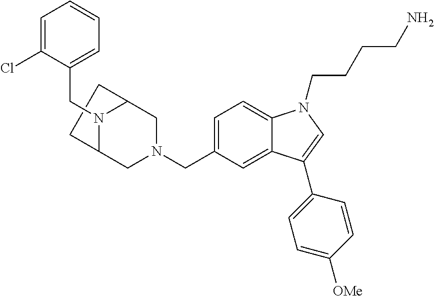 [SMILES: COc1ccc(-c2cn(CCCCN)c3ccc(CN4CC5CCC(C4)N5Cc4ccccc4Cl)cc23)cc1] | 4-(5-((8-(2-chlorobenzyl)- 3,8-diazabicyclo[3.2.1]octan- 3-yl)methyl)-3-(4- methoxyphenyl)-1H-indol-1- yl)butan-1-amine |
| C157 | 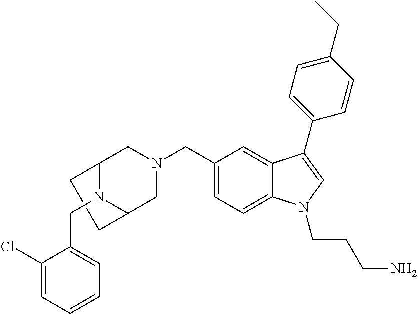 [SMILES: CCc1ccc(-c2cn(CCCN)c3ccc(CN4CC5CCC(C4)N5Cc4ccccc4Cl)cc23)cc1] | 3-(5-((8-(2-chlorobenzyl)- 3,8-diazabicyclo[3.2.1]octan- 3-yl)methyl)-3-(4- ethylphenyl)-1H-indol-1- yl)propan-1-amine |
| C158 | 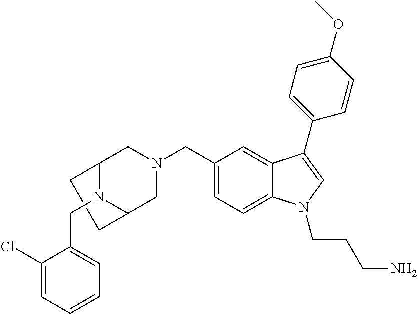 [SMILES: COc1ccc(-c2cn(CCCN)c3ccc(CN4CC5CCC(C4)N5Cc4ccccc4Cl)cc23)cc1] | 3-(5-((8-(2-chlorobenzyl)- 3,8-diazabicyclo[3.2.1]octan- 3-yl)methyl)-3-(4- methoxyphenyl)-1H-indol-1- yl)propan-1-amine |
| C159 | 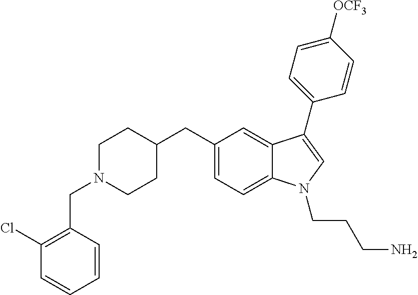 [SMILES: NCCCn1cc(-c2ccc(OC(F)(F)F)cc2)c2cc(CC3CCN(Cc4ccccc4Cl)CC3)ccc21] | 3-(5-((1-(2- chlorobenzyl)piperidin-4- yl)methyl)-3-(4- (trifluoromethoxy)phenyl)- 1H-indol-1-yl)propan-1- amine |
| C169 | 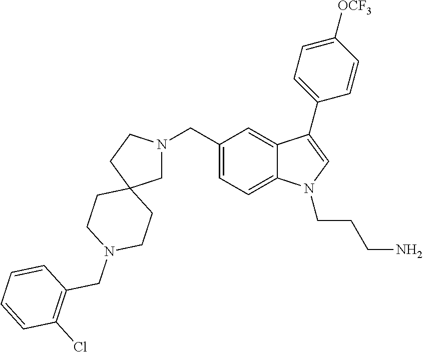 [SMILES: NCCCn1cc(-c2ccc(OC(F)(F)F)cc2)c2cc(CN3CCC4(CCN(Cc5ccccc5Cl)CC4)C3)ccc21] | 3-(5-((8-(2-chlorobenzyl)- 2,8-diazaspiro[4.5]decan-2- yl)methyl)-3-(4- (trifluoromethoxy)phenyl)- 1H-indol-1-yl)propan-1- amine |

In some embodiments, the compound of Formula (Va) is selected from any one of the compounds from the following table:

TABLE 9B

| Compound | | |
|---|---|---|
| Ref. | Compound | Chemical Name |
| C210 | 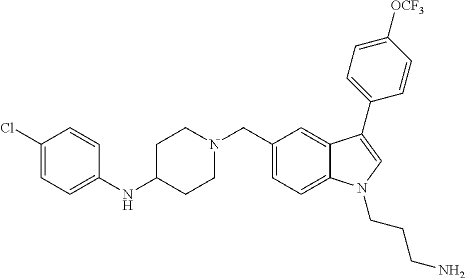 [SMILES: NCCCn1cc(-c2ccc(OC(F)(F)F)cc2)c2cc(CN3CCC(Nc4ccc(Cl)cc4)CC3)ccc21] | 1-((1-(3-aminopropyl)-3-(4- (trifluoromethoxy)phenyl)- 1H-indol-5-yl)methyl)-N-(4- chlorophenyl)piperidin-4- amine |
| C213 | 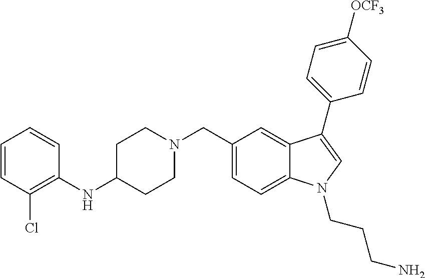 [SMILES: NCCCn1cc(-c2ccc(OC(F)(F)F)cc2)c2cc(CN3CCC(Nc4ccccc4Cl)CC3)ccc21] | 1-((1-(3-aminopropyl)-3-(4- (trifluoromethoxy)phenyl)- 1H-indol-5-yl)methyl)-N-(2- chlorophenyl)piperidin-4- amine |
| C214 | 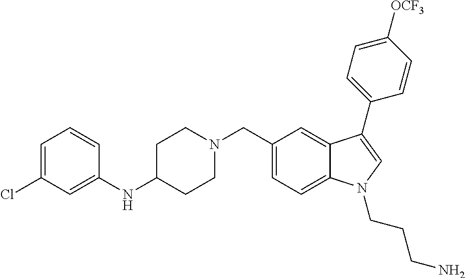 [SMILES: NCCCn1cc(-c2ccc(OC(F)(F)F)cc2)c2cc(CN3CCC(Nc4cccc(Cl)c4)CC3)ccc21] | 1-((1-(3-aminopropyl)-3-(4- (trifluoromethoxy)phenyl)- 1H-indol-5-yl)methyl)-N-(3- chlorophenyl)piperidin-4- amine |
| C215 | 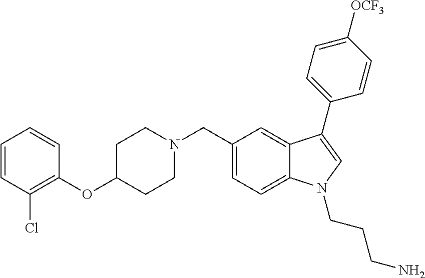 [SMILES: NCCCn1cc(-c2ccc(OC(F)(F)F)cc2)c2cc(CN3CCC(Oc4ccccc4Cl)CC3)ccc21] | 3-(5-((4-(2- chlorophenoxy)piperidin-1- yl)methyl)-3-(4- (trifluoromethoxy)phenyl)- 1H-indol-1-yl)propan-1- amine |
| C216 | 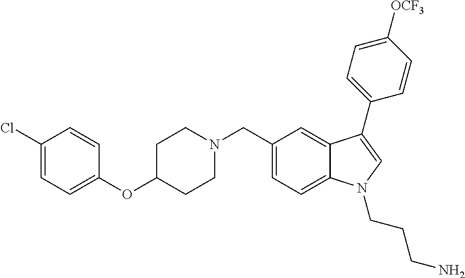 [SMILES: NCCCn1cc(-c2ccc(OC(F)(F)F)cc2)c2cc(CN3CCC(Oc4ccc(Cl)cc4)CC3)ccc21] | 3-(5-((4-(4- chlorophenoxy)piperidin-1- yl)methyl)-3-(4- (trifluoromethoxy)phenyl)- 1H-indol-1-yl)propan-1- amine |
| C221 | 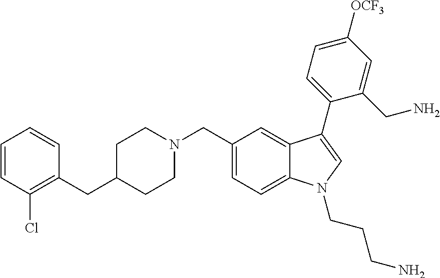 [SMILES: NCCCn1cc(-c2ccc(OC(F)(F)F)cc2CN)c2cc(CN3CCC(Cc4ccccc4Cl)CC3)ccc21] | 3-(3-(2-(aminomethyl)-4- (trifluoromethoxy)phenyl)-5- ((4-(2- chlorobenzyl)piperidin-1- yl)methyl)-1H-indol-1- yl)propan-1-amine |
| C226 | 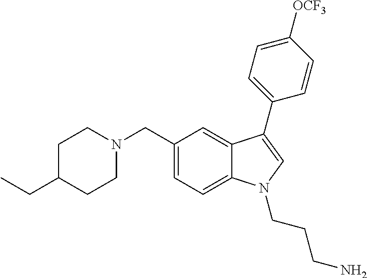 [SMILES: CCC1CCN(Cc2ccc3c(c2)c(-c2ccc(OC(F)(F)F)cc2)cn3CCCN)CC1] | 3-(5-((4-ethylpiperidin-1- yl)methyl)-3-(4- (trifluoromethoxy)phenyl)- 1H-indol-1-yl)propan-1- amine |
| C233 | 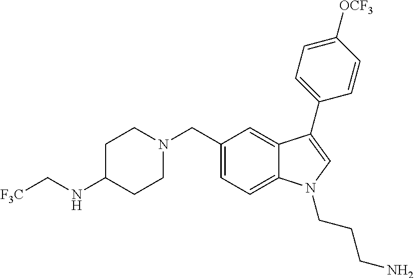 [SMILES: NCCCn1cc(-c2ccc(OC(F)(F)F)cc2)c2cc(CN3CCC(NCC(F)(F)F)CC3)ccc21] | 1-((1-(3-aminopropyl)-3-(4- (trifluoromethoxy)phenyl)- 1H-indol-5-yl)methyl)-N- (2,2,2- trifluoroethyl)piperidin-4- amine |
| C236 | 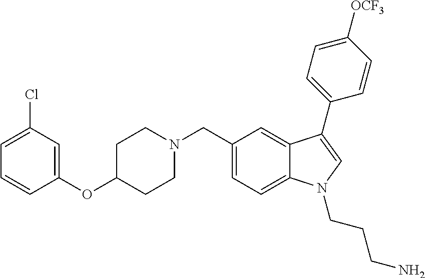 [SMILES: NCCCn1cc(-c2ccc(OC(F)(F)F)cc2)c2cc(CN3CCC(Oc4cccc(Cl)c4)CC3)ccc21] | 3-(5-((4-(3- chlorophenoxy)piperidin-1- yl)methyl)-3-(4- (trifluoromethoxy)phenyl)- 1H-indol-1-yl)propan-1- amine |
| C241 | 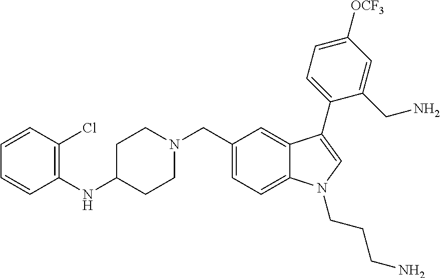 [SMILES: NCCCn1cc(-c2ccc(OC(F)(F)F)cc2CN)c2cc(CN3CCC(Nc4ccccc4Cl)CC3)ccc21] | 1-((3-(2-(aminomethyl)-4- (trifluoromethoxy)phenyl)-1- (3-aminopropyl)-1H-indol-5- yl)methyl)-N-(2- chlorophenyl)piperidin-4- amine |
| C242 | 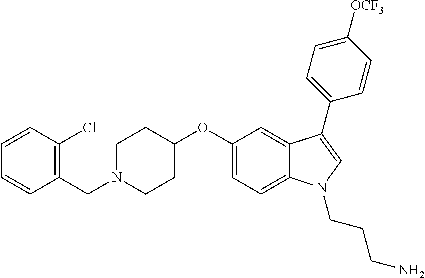 [SMILES: NCCCn1cc(-c2ccc(OC(F)(F)F)cc2)c2cc(OC3CCN(Cc4ccccc4Cl)CC3)ccc21] | 3-(5-((1-(2- chlorobenzyl)piperidin-4- yl)oxy)-3-(4- (trifluoromethoxy)phenyl)- 1H-indol-1-yl)propan-1- amine |
| C247 | 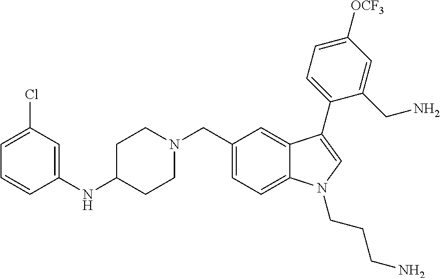 [SMILES: NCCCn1cc(-c2ccc(OC(F)(F)F)cc2CN)c2cc(CN3CCC(Nc4cccc(Cl)c4)CC3)ccc21] | 1-((3-(2-(aminomethyl)-4- (trifluoromethoxy)phenyl)-1- (3-aminopropyl)-1H-indol-5- yl)methyl)-N-(3- chlorophenyl)piperidin-4- amine |
| C249 | 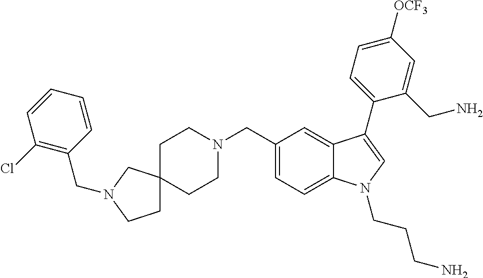 [SMILES: NCCCn1cc(-c2ccc(OC(F)(F)F)cc2CN)c2cc(CN3CCC4(CC3)CCN(Cc3ccccc3Cl)C4)ccc21] | 3-(3-(2-(aminomethyl)-4- (trifluoromethoxy)phenyl)-5- ((2-(2-chlorobenzyl)-2,8- diazaspiro[4.5]decan-8- yl)methyl)-1H-indol-1- yl)propan-1-amine |
| C250 | 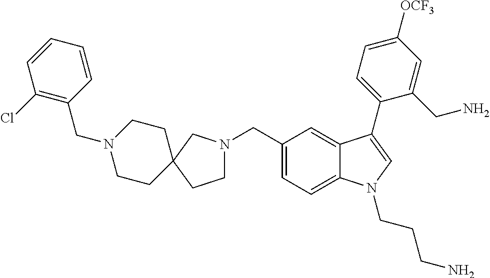 [SMILES: NCCCn1cc(-c2ccc(OC(F)(F)F)cc2CN)c2cc(CN3CCC4(CCN(Cc5ccccc5Cl)CC4)C3)ccc21] | 3-(3-(2-(aminomethyl)-4- (trifluoromethoxy)phenyl)-5- ((8-(2-chlorobenzyl)-2,8- diazaspiro[4.5]decan-2- yl)methyl)-1H-indol-1- yl)propan-1-amine |
| C252 | 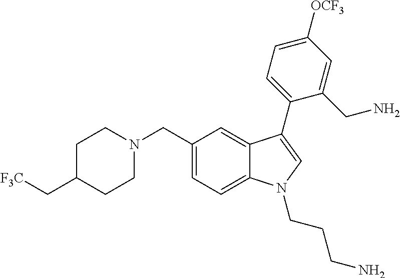 [SMILES: NCCCn1cc(-c2ccc(OC(F)(F)F)cc2CN)c2cc(CN3CCC(CC(F)(F)F)CC3)ccc21] | 3-(3-(2-(aminomethyl)-4- (trifluoromethoxy)phenyl)-5- ((4-(2,2,2- trifluoroethyl)piperidin-1- yl)methyl)-1H-indol-1- yl)propan-1-amine |
| C253 | 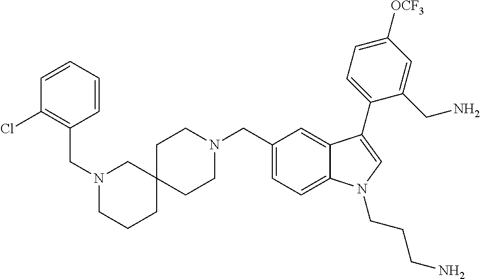 [SMILES: NCCCn1cc(-c2ccc(OC(F)(F)F)cc2CN)c2cc(CN3CCC4(CCCN(Cc5ccccc5Cl)C4)CC3)ccc21] | 3-(3-(2-(aminomethyl)-4- (trifluoromethoxy)phenyl)-5- ((2-(2-chlorobenzyl)-2,9- diazaspiro[5.5]undecan-9- yl)methyl)-1H-indol-1- yl)propan-1-amine |
| C255 | 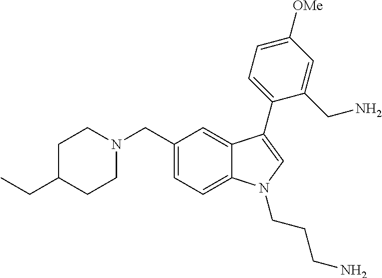 [SMILES: CCC1CCN(Cc2ccc3c(c2)c(-c2ccc(OC)cc2CN)cn3CCCN)CC1] | 3-(3-(2-(aminomethyl)-4- methoxyphenyl)-5-((4- ethylpiperidin-1-yl)methyl)- 1H-indol-1-yl)propan-1- amine |
| C256 | 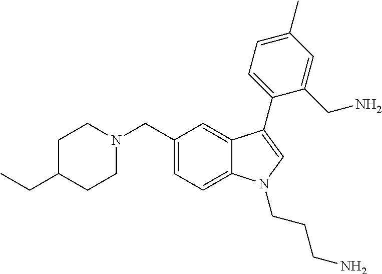 [SMILES: CCC1CCN(Cc2ccc3c(c2)c(-c2ccc(C)cc2CN)cn3CCCN)CC1] | 3-(3-(2-(aminomethyl)-4- methylphenyl)-5-((4- ethylpiperidin-1-yl)methyl)- 1H-indol-1-yl)propan-1- amine |
| C257 | 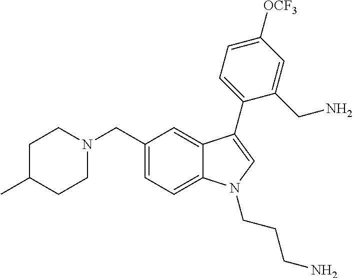 [SMILES: CC1CCN(Cc2ccc3c(c2)c(-c2ccc(OC(F)(F)F)cc2CN)cn3CCCN)CC1] | 3-(3-(2-(aminomethyl)-4- (trifluoromethoxy)phenyl)-5- ((4-methylpiperidin-1- yl)methyl)-1H-indol-1- yl)propan-1-amine |
| C258 | 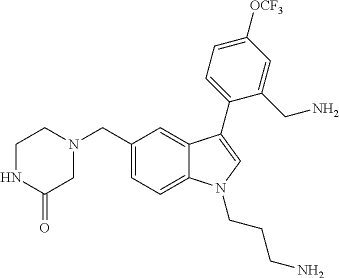 [SMILES: NCCCn1cc(-c2ccc(OC(F)(F)F)cc2CN)c2cc(CN3CCNC(=O)C3)ccc21] | 4-((3-(2-(aminomethyl)-4- (trifluoromethoxy)phenyl)-1- (3-aminopropyl)-1H-indol-5- yl)methyl)piperazin-2-one |
| C259 | 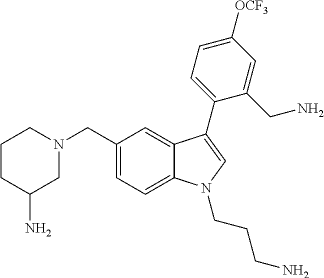 [SMILES: NCCCn1cc(-c2ccc(OC(F)(F)F)cc2CN)c2cc(CN3CCCC(N)C3)ccc21] | 1-((3-(2-(aminomethyl)-4- (trifluoromethoxy)phenyl)-1- (3-aminopropyl)-1H-indol-5- yl)methyl)piperidin-3-amine |
| C260 | 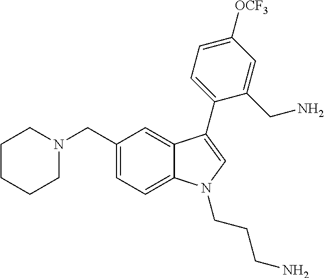 [SMILES: NCCCn1cc(-c2ccc(OC(F)(F)F)cc2CN)c2cc(CN3CCCCC3)ccc21] | 3-(3-(2-(aminomethyl)-4- (trifluoromethoxy)phenyl)-5- (piperidin-1-ylmethyl)-1H- indol-1-yl)propan-1-amine |
| C261 | 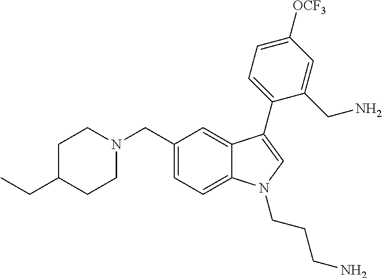 [SMILES: CCC1CCN(Cc2ccc3c(c2)c(-c2ccc(OC(F)(F)F)cc2CN)cn3CCCN)CC1] | 3-(3-(2-(aminomethyl)-4- (trifluoromethoxy)phenyl)-5- ((4-ethylpiperidin-1- yl)methyl)-1H-indol-1- yl)propan-1-amine |
| C262 | 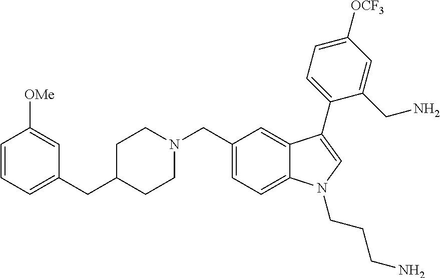 [SMILES: COc1cccc(CC2CCN(Cc3ccc4c(c3)c(-c3ccc(OC(F)(F)F)cc3CN)cn4CCCN)CC2)c1] | 3-(3-(2-(aminomethyl)-4- (trifluoromethoxy)phenyl)-5- ((4-(3- methoxybenzyl)piperidin-1- yl)methyl)-1H-indol-1- yl)propan-1-amine |
| C263 | 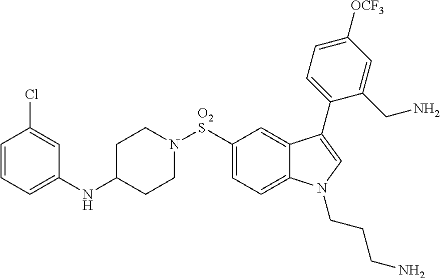 [SMILES: NCCCn1cc(-c2ccc(OC(F)(F)F)cc2CN)c2cc(S(=O)(=O)N3CCC(Nc4cccc(Cl)c4)CC3)ccc21] | 1-((3-(2-(aminomethyl)-4- (trifluoromethoxy)phenyl)-1- (3-aminopropyl)-1H-indol-5- yl)sulfonyl)-N-(3- chlorophenyl)piperidin-4- amine |
| C265 | 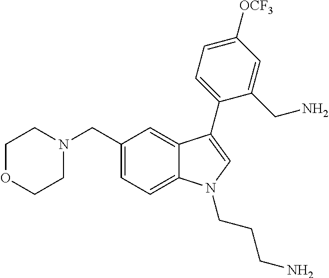 [SMILES: NCCCn1cc(-c2ccc(OC(F)(F)F)cc2CN)c2cc(CN3CCOCC3)ccc21] | 3-(3-(2-(aminomethyl)-4- (trifluoromethoxy)phenyl)-5- (morpholinomethyl)-1H- indol-1-yl)propan-1-amine |
| C269 | 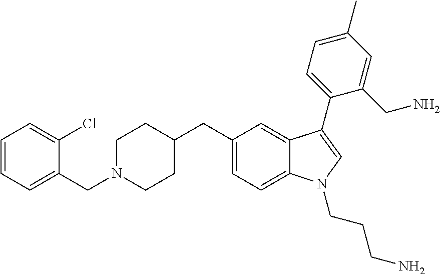 [SMILES: Cc1ccc(-c2cn(CCCN)c3ccc(CC4CCN(Cc5ccccc5Cl)CC4)cc23)c(CN)c1] | 3-(3-(2-(aminomethyl)-4- methylphenyl)-5-((1-(2- chlorobenzyl)piperidin-4- yl)methyl)-1H-iundol-1- yl)propan-1-amine |
| C270 | 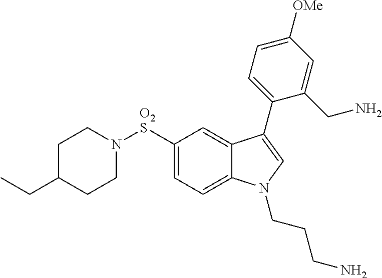 [SMILES: CCC1CCN(S(=O)(=O)c2ccc3c(c2)c(-c2ccc(OC)cc2CN)cn3CCCN)CC1] | 3-(3-(2-(aminomethyl)-4- methoxyphenyl)-5-((4- ethylpiperidin-1-yl)sulfonyl)- 1H-indol-1-yl)propan-1- amine |
| C272 | 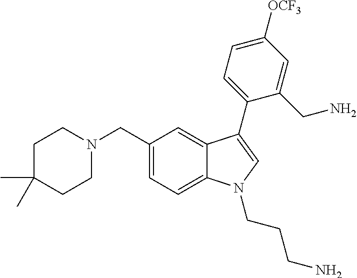 [SMILES: CC1(C)CCN(Cc2ccc3c(c2)c(-c2ccc(OC(F)(F)F)cc2CN)cn3CCCN)CC1] | 3-(3-(2-(aminomethyl)-4- (trifluoromethoxy)phenyl)-5- ((4,4-dimethylpiperidin-1- yl)methyl)-1H-indol-1- yl)propan-1-amine |
| C273 | 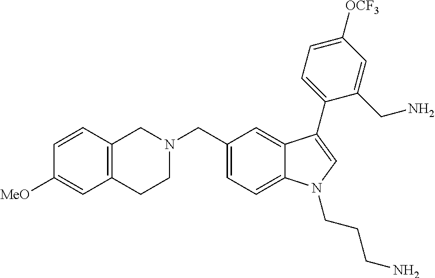 [SMILES: COc1ccc2c(c1)CCN(Cc1ccc3c(c1)c(-c1ccc(OC(F)(F)F)cc1CN)cn3CCCN)C2] | 3-(3-(2-(aminomethyl)-4- (trifluoromethoxy)phenyl)-5- ((6-methoxy-3,4- dihydroisoquinolin-2(1H)- yl)methyl)-1H-indol-1- yl)propan-1-amine |
| C276 | 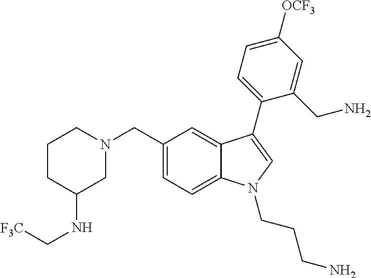 [SMILES: NCCCn1cc(-c2ccc(OC(F)(F)F)cc2CN)c2cc(CN3CCCC(NCC(F)(F)F)C3)ccc21] | 1-((3-(2-(aminomethyl)-4- (trifluoromethoxy)phenyl)-1- (3-aminopropyl)-1H-indol-5- yl)methyl)-N-(2,2,2- trifluoroethyl)piperidin-3- amine |
| C277 | 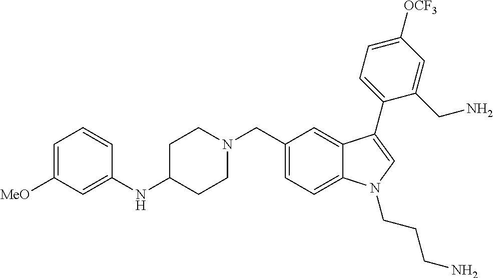 [SMILES: COc1cccc(NC2CCN(Cc3ccc4c(c3)c(-c3ccc(OC(F)(F)F)cc3CN)cn4CCCN)CC2)c1] | 1-((3-(2-(aminomethyl)-4- (trifluoromethoxy)phenyl)-1[- (3-aminopropyl)-1H-indol-5- yl)methyl)-N-(3- methoxyphenyl)piperidin-4- amine |
| C278 | 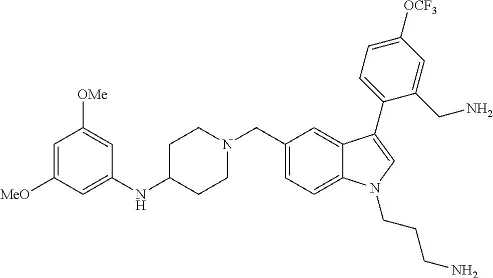 [SMILES: COc1cc(NC2CCN(Cc3ccc4c(c3)c(-c3ccc(OC(F)(F)F)cc3CN)cn4CCCN)CC2)cc(OC)c1] | 1-((3-(2-(aminomethyl)-4- (trifluoromethoxy)phenyl)-1- (3-aminopropyl)-1H-indol-5- yl)methyl)-N-(3,5- dimethoxyphenyl)piperidin- 4-amine |
| C279 | 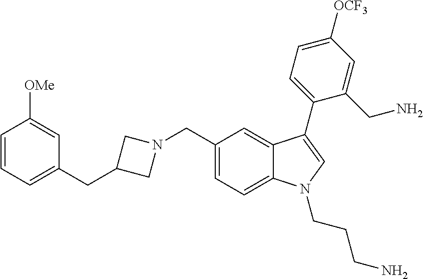 [SMILES: COc1cccc(CC2CN(Cc3ccc4c(c3)c(-c3ccc(OC(F)(F)F)cc3CN)cn4CCCN)C2)c1] | 3-(3-(2-(aminomethyl)-4- (trifluoromethoxy)phenyl)-5- ((3-(3- methoxybenzyl)azetidin-1- yl)methyl)-1H-indol-1- yl)propan-1-amine |
| C280 | 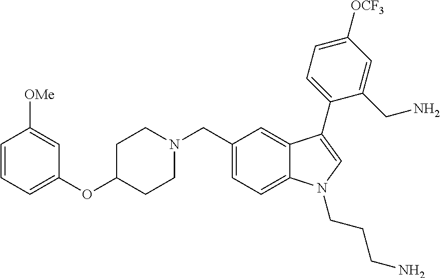 [SMILES: COc1cccc(OC2CCN(Cc3ccc4c(c3)c(-c3ccc(OC(F)(F)F)cc3CN)cn4CCCN)CC2)c1] | 3-(3-(2-(aminomethyl)-4- (trifluoromethoxy)phenyl)-5- ((4-(3- methoxyphenoxy)piperidin- 1-yl)methyl)-1H-indol-1- yl)propan-1-amine |
| C281 | 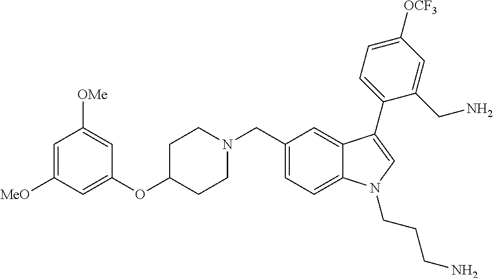 [SMILES: COc1cc(OC)cc(OC2CCN(Cc3ccc4c(c3)c(-c3ccc(OC(F)(F)F)cc3CN)cn4CCCN)CC2)c1] | 3-(3-(2-(aminomethyl)-4- (trifluoromethoxy)phenyl)-5- ((4-(3,5- dimethoxyphenoxy)piperidin- 1-yl)methyl)-1H-inol-1- yl)propan-1-amine |
| C282 | 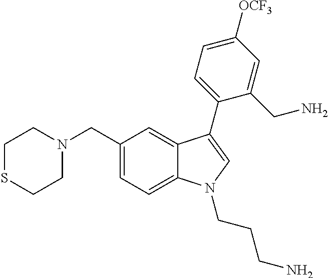 [SMILES: NCCCn1cc(-c2ccc(OC(F)(F)F)cc2CN)c2cc(CN3CCSCC3)ccc21] | 3-(3-(2-(aminomethyl)-4- (trifluoromethoxy)phenyl)-5- (thiomorpholinomethyl)-1H- indol-1-yl)propan-1-amine |
| C286 | 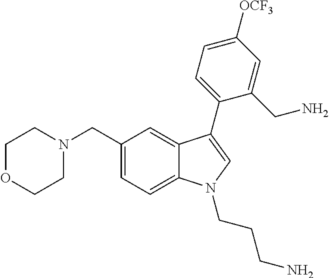 [SMILES: NCCCn1cc(-c2ccc(OC(F)(F)F)cc2CN)c2cc(CN3CCOCC3)ccc21] | 4-(5-(morpholinomethyl)-4- (4- (trifluoromethoxy)phenyl)- 1H-indol-1-yl)propan-1- amine |
| C288 | 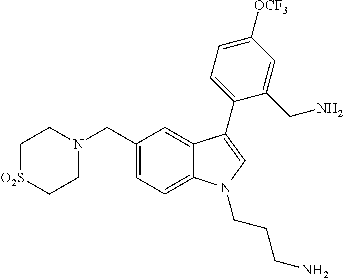 [SMILES: NCCCn1cc(-c2ccc(OC(F)(F)F)cc2CN)c2cc(CN3CCS(=O)(=O)CC3)ccc21] | 4-((3-(2-(aminomethyl)-4- (trifluoromethoxy)phenyl)-1- (3-aminopropyl)-1H-indol-5- yl)methyl)thiomorpholine 1,1-dioxide |
| C290 | 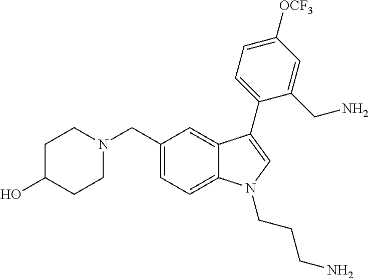 [SMILES: NCCCn1cc(-c2ccc(OC(F)(F)F)cc2CN)c2cc(CN3CCC(O)CC3)ccc21] | 1-((3-(2-(aminomethyl)-4- (trifluoromethoxy)phenyl)-1- (3-aminopropyl)-1H-indol-5- yl)methyl)piperidin-4-ol |
| C291 | 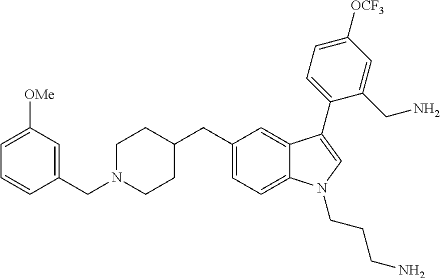 [SMILES: COc1cccc(CN2CCC(Cc3ccc4c(c3)c(-c3ccc(OC(F)(F)F)cc3CN)cn4CCCN)CC2)c1] | 3-(3-(2-(aminomethyl)-4- (trifluoromethoxy)phenyl)-5- ((1-(3- methoxybenzyl)piperidin-4- yl)methyl)-1H-indol-1- yl)propan-1-amine |
| C292 | 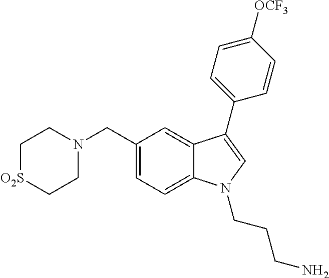 [SMILES: NCCCn1cc(-c2ccc(OC(F)(F)F)cc2)c2cc(CN3CCS(=O)(=O)CC3)ccc21] | 4-((1-(3-aminopropyl)-3-(4- (trifluoromethoxy)phenyl)- 1H-indol-5- yl)methyl)thiomorpholine 1,1-dioxide |
| C293 | 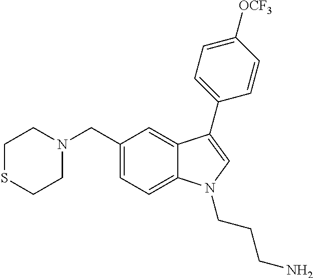 [SMILES: NCCCn1cc(-c2ccc(OC(F)(F)F)cc2)c2cc(CN3CCSCC3)ccc21] | 3-(5- (thiomorpholinomethyl)-3- (4- (trifluoronmethoxy)phenyl)- 1H-indol-1-yl)propan-1- amine |
| C294 | 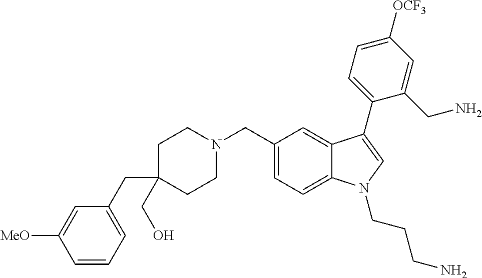 [SMILES: COc1cccc(CC2(CO)CCN(Cc3ccc4c(c3)c(-c3ccc(OC(F)(F)F)cc3CN)cn4CCCN)CC2)c1] | (1-((3-(2-(aminomethyl)-4- (trifluoromethoxy)phenyl)-1- (3-aminopropyl)-1H-indol-5- yl)methyl)-4-(3- methoxybenzyl)piperidin-4- yl)methanol |
| C295 | 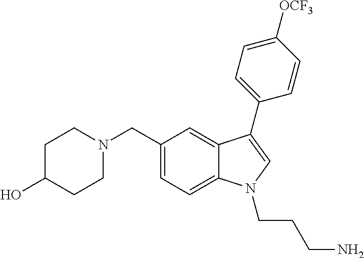 [SMILES: NCCCn1cc(-c2ccc(OC(F)(F)F)cc2)c2cc(CN3CCC(O)CC3)ccc21] | 1-((1-(3-aminopropyl)-3-(4- (trifluoromethoxy)phenyl)- 1H-indol-5- yl)methyl)piperidin-4-ol |
| C308 | 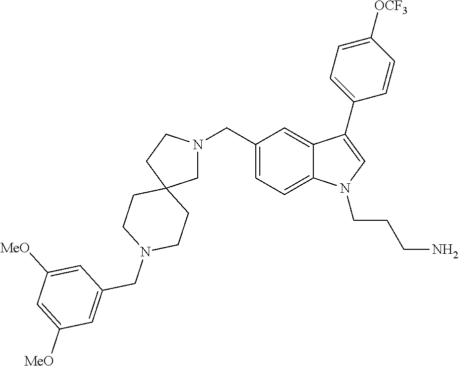 [SMILES: COc1cc(CN2CCC3(CC2)CCN(Cc2ccc4c(c2)c(-c2ccc(OC(F)(F)F)cc2)cn4CCCN)C3)cc(OC)c1] | 3-(5-((8-(3,5- dimethoxybenzyl)-2,8- diazsaspiro[4.5]decan-2- yl)methyl)-3-(4- (trifluoromethoxy)phenyl)- 1H-indol-1-yl)propan-1- amine |
| C309 | 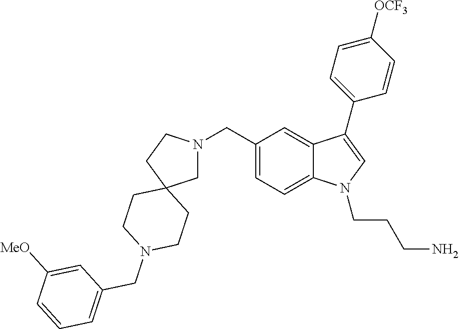 [SMILES: COc1cccc(CN2CCC3(CC2)CCN(Cc2ccc4c(c2)c(-c2ccc(OC(F)(F)F)cc2)cn4CCCN)C3)c1] | 3-(5-((8-(3-methoxybenzyl)- 2,8-diazaspiro[4.5]decan-2- yl)methyl)-3-(4- (trifluoromethoxy)phenyl)- 1H-indol-1-yl)propan-1- amine |

In another aspect provided herein is a compound of Formula (VIa), or a pharmaceutically acceptable salt, or solvate thereof:

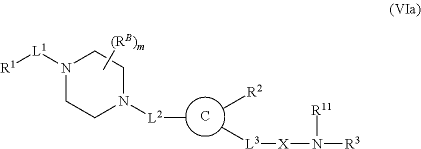

wherein,

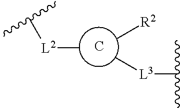

is a bicyclic heteroaryl that is selected from the following structures:

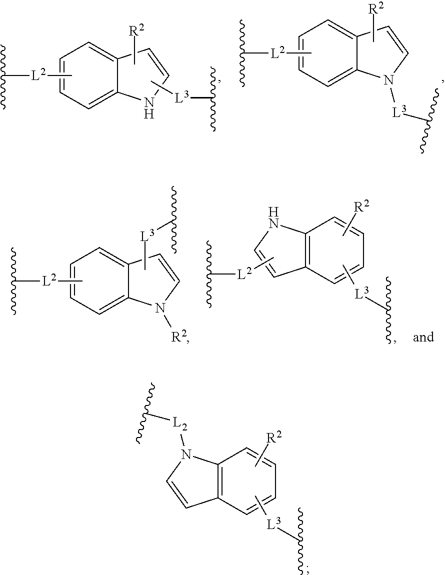

L¹ and L² are each independently absent, an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
each R^{B} is independently optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, a substituted C₁-C₆alkylene, an optionally substituted phenylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
R³ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, —C₁-C₄alkylene-(optionally substituted heteroaryl), —CH₂C(═O)R¹⁵, —C(═O)R¹⁵, or —CO₂R¹⁶;
R⁴ is hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹⁵ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁶ is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each m is independently 0, 1, 2, 3, or 4.

In some embodiments,

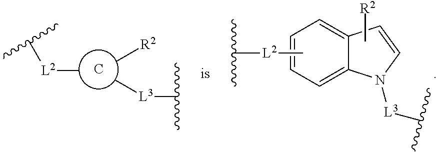

In some embodiments,

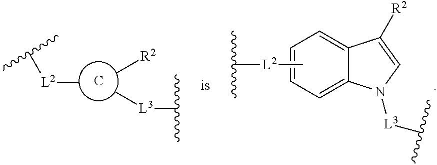

In some embodiments, the compound has the following structure of Formula (VIb), or a pharmaceutically acceptable salt, or solvate thereof:

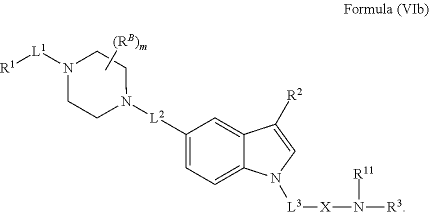

In some embodiments, the compound has the following structure of Formula (VIc), or a pharmaceutically acceptable salt, or solvate thereof:

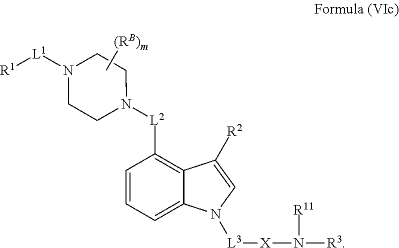

In some embodiments,

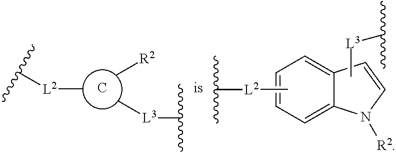

In some embodiments,

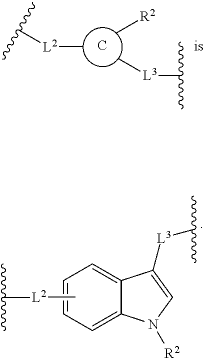

In some embodiments, the compound has the following structure of Formula (VId), or a pharmaceutically acceptable salt, or solvate thereof:

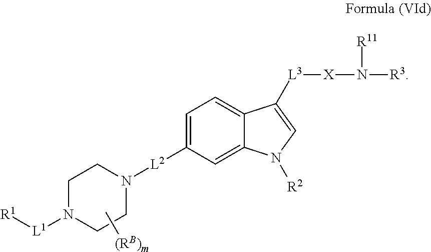

In some embodiments, the compound has the following structure of Formula (VIe), or a pharmaceutically acceptable salt, or solvate thereof:

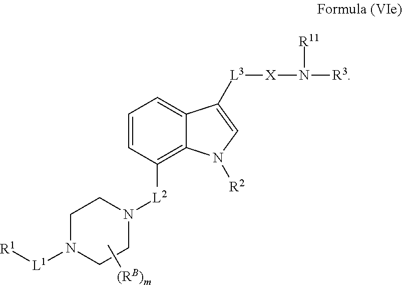

In some embodiments,

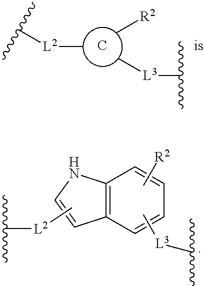

In some embodiments, L¹ is —CH₂—, C(═O), O, S, S(═O), S(═O)₂, or NR⁴. In some embodiments, L¹ is —CH₂—. In some embodiments, L² is —CH₂—, C(═O), O, S, S(═O), S(═O)₂, or NR⁴. In some embodiments, L² is —CH₂—. In some embodiments, L³ is a substituted C₁-C₅alkylene.

In some embodiments, L³ is:

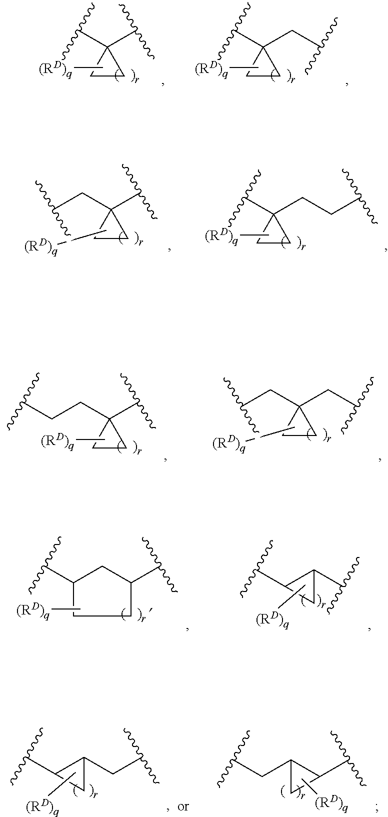

each q is independently 0, 1, 2, 3, or 4;

r is 1, 2, 3, or 4; and

r′ is 1 or 2.

In some embodiments, X is —CH₂— or C(═O). In some embodiments, X is —CH₂—.

In some embodiments, X is:

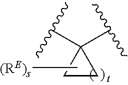

each s is independently 0, 1, 2, 3, or 4; and

t is 1, 2, 3, or 4.

In some embodiments, X is:

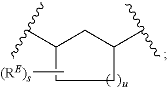

each s is independently 0, 1, 2, 3, or 4; and

u is 0, 1, or 2.

In some embodiments, R¹¹ and R³ are each independently hydrogen, optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹¹ and R³ are each hydrogen. In some embodiments, R¹¹ and R³ are each optionally substituted C₁-C₆alkyl. In some embodiments, R¹¹ and R³ are each optionally substituted C₁-C₆heteroalkyl. In some embodiments, R¹⁵ and R¹⁶ are each independently optionally substituted C₁-C₆alkyl or optionally substituted C₁-C₆heteroalkyl. In some embodiments, R¹¹ is hydrogen and R³ is optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹ is an unsubstituted phenyl. In some embodiments, R¹ is a substituted phenyl. In some embodiments, R¹ is selected from:

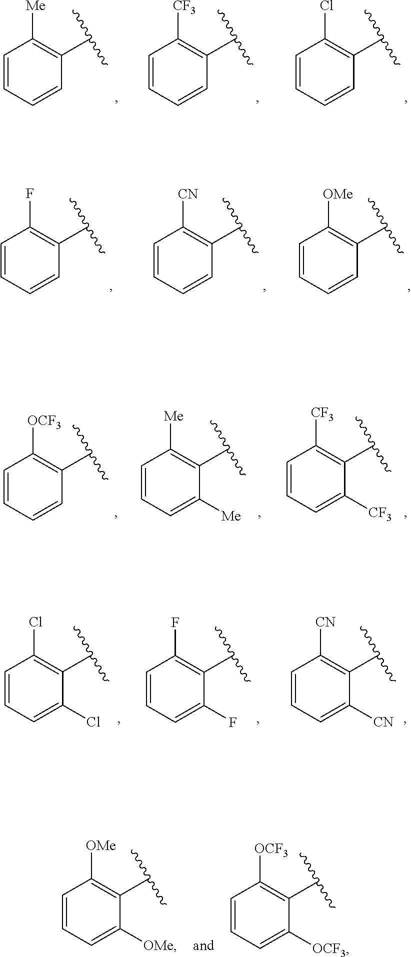

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

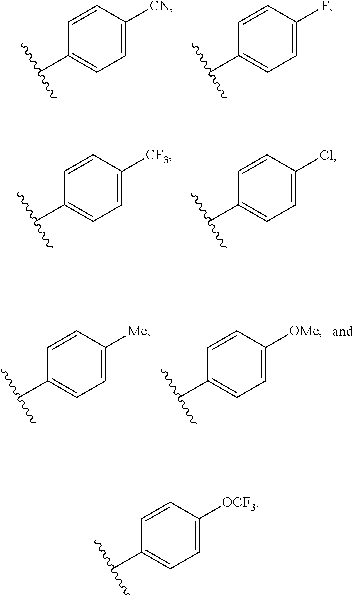

In some embodiments, R² is selected from:

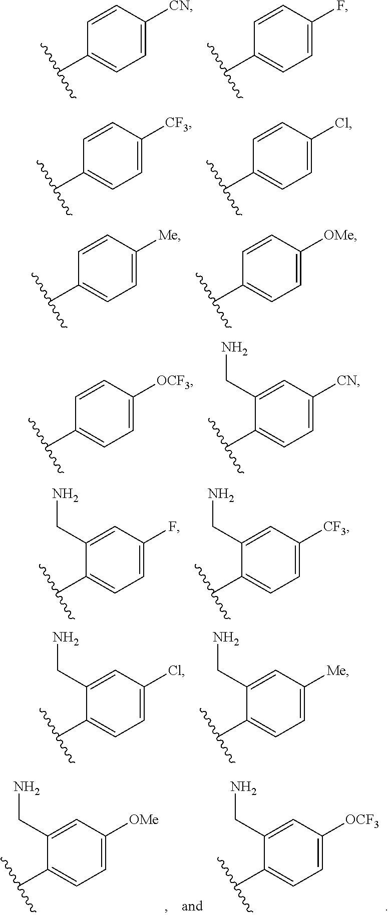

In some embodiments, R¹ is optionally substituted heterocycloalkyl. In some embodiments, R¹ is selected from:

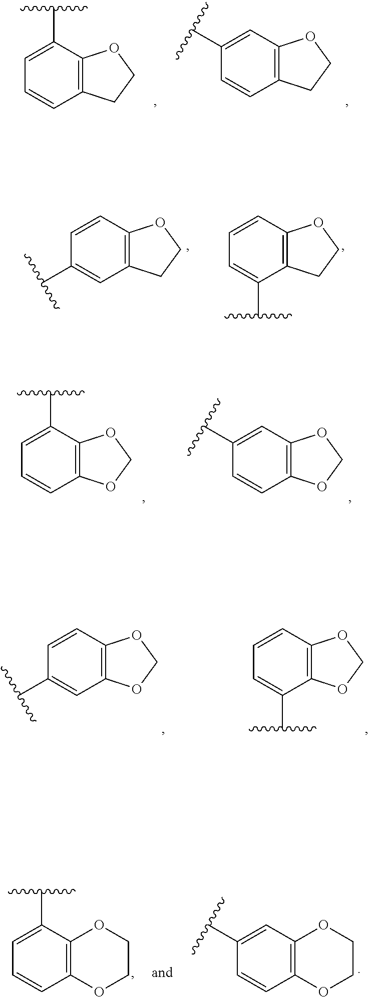

In some embodiments, R² is optionally substituted heterocycloalkyl. In some embodiments, R² is selected from:

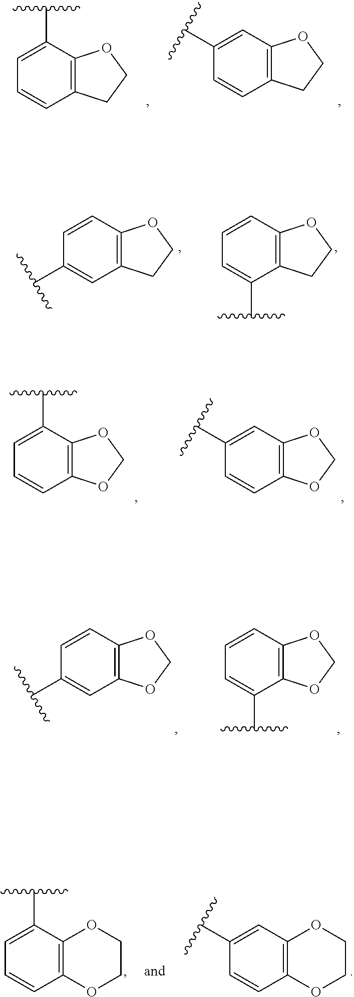

In some embodiments, the compound of Formula (VIa) is selected from:

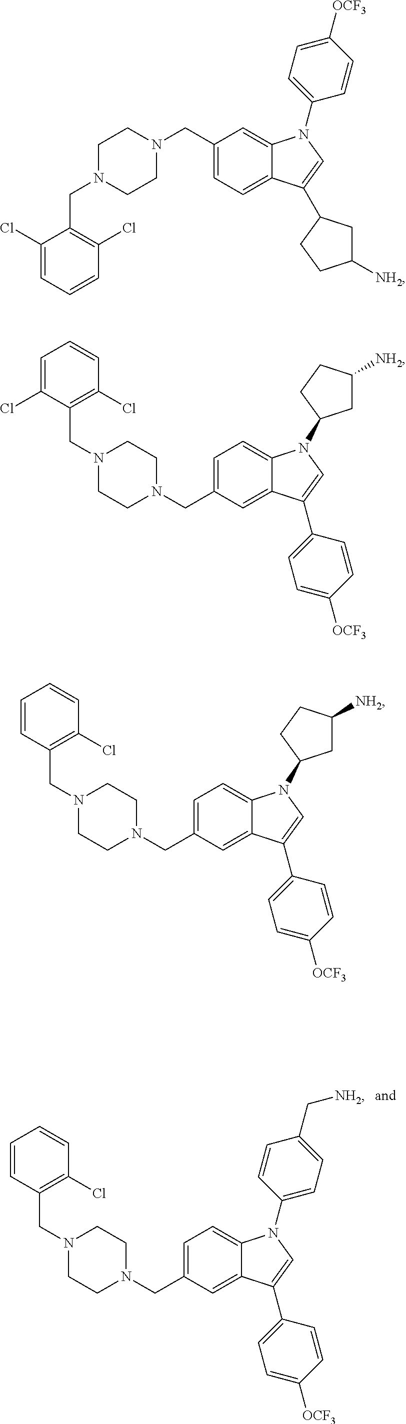 [SMILES: NC1CCC(c2cn(-c3ccc(OC(F)(F)F)cc3)c3cc(CN4CCN(Cc5c(Cl)cccc5Cl)CC4)ccc23)C1 ; N[C@H]1CC[C@H](n2cc(-c3ccc(OC(F)(F)F)cc3)c3cc(CN4CCN(Cc5c(Cl)cccc5Cl)CC4)ccc32)C1 ; N[C@@H]1CC[C@H](n2cc(-c3ccc(OC(F)(F)F)cc3)c3cc(CN4CCN(Cc5ccccc5Cl)CC4)ccc32)C1 ; NCc1ccc(-n2cc(-c3ccc(OC(F)(F)F)cc3)c3cc(CN4CCN(Cc5ccccc5Cl)CC4)ccc32)cc1 ; "and"]

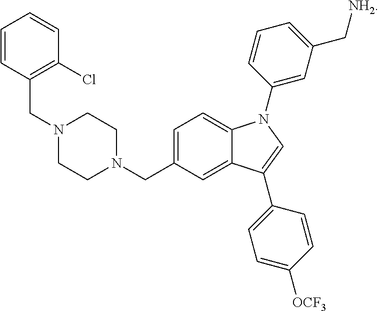 [SMILES: NCc1cccc(-n2cc(-c3ccc(OC(F)(F)F)cc3)c3cc(CN4CCN(Cc5ccccc5Cl)CC4)ccc32)c1]

In some embodiments, the compound of Formula (VIa) is selected from:

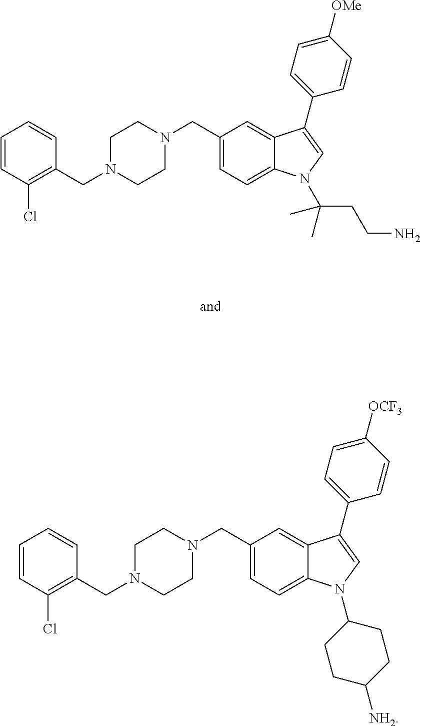 [SMILES: COc1ccc(-c2cn(C(C)(C)CCN)c3ccc(CN4CCN(Cc5ccccc5Cl)CC4)cc23)cc1 ; NC1CCC(n2cc(-c3ccc(OC(F)(F)F)cc3)c3cc(CN4CCN(Cc5ccccc5Cl)CC4)ccc32)CC1]

In some embodiments, the compound of Formula (VIa) is selected from any one of the compounds from the following table:

TABLE 10A

| Compound | | |
|---|---|---|
| Ref. | Compound | Chemical Name |
| C30 | 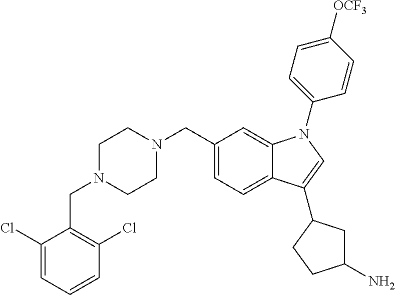 [SMILES: NC1CCC(c2cn(-c3ccc(OC(F)(F)F)cc3)c3cc(CN4CCN(Cc5c(Cl)cccc5Cl)CC4)ccc23)C1] | 3-(6-((4-(2,6- dichlorobenzyl)piperazin-1- yl)methyl)-1-(4- (trifluoromethoxy)phenyl)- 1H-indol-3- yl)cyclopenanamine |
| C42 | 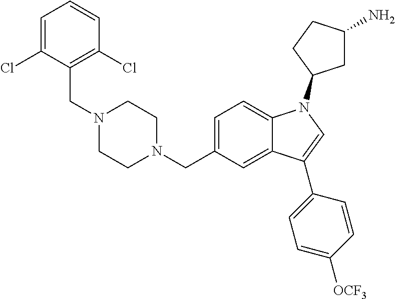 [SMILES: N[C@H]1CC[C@H](n2cc(-c3ccc(OC(F)(F)F)cc3)c3cc(CN4CCN(Cc5c(Cl)cccc5Cl)CC4)ccc32)C1] | (1S,3S)-3-(5-((4-(2,6- dichlorobenzyl)piperazin-1- yl)methyl)-3-(4- (trifluoromethoxy)phenyl)- 1H-indol-1- yl)cyclopentanamine |
| C134 | 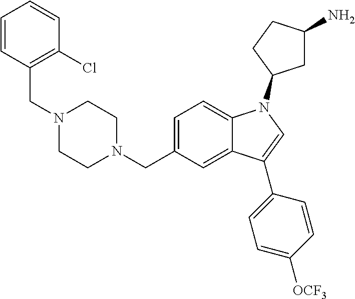 [SMILES: N[C@@H]1CC[C@H](n2cc(-c3ccc(OC(F)(F)F)cc3)c3cc(CN4CCN(Cc5ccccc5Cl)CC4)ccc32)C1] | (1R,3S)-3-(5-((4-(2- chlorobenzyl)piperazin-1- yl)methyl)-3-(4- (trifluoromethoxy)phenyl)- 1H-indol-1- yl)cyclopentanamine |
| C149 | 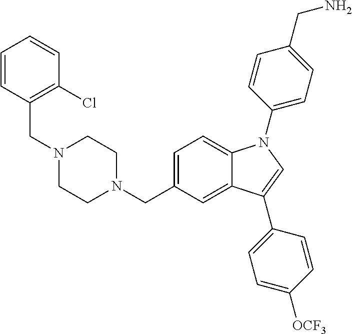 [SMILES: NCc1ccc(-n2cc(-c3ccc(OC(F)(F)F)cc3)c3cc(CN4CCN(Cc5ccccc5Cl)CC4)ccc32)cc1] | (4-(5-((4-(2- chlorobenzyl)piperazin-1- yl)methyl)-3-(4- (trifluoromethoxy)phenyl)- 1H-indol-1- yl)phenyl)methanamine |
| C150 | 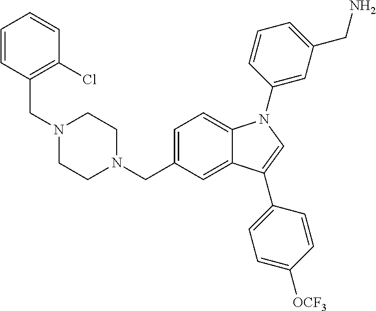 [SMILES: NCc1cccc(-n2cc(-c3ccc(OC(F)(F)F)cc3)c3cc(CN4CCN(Cc5ccccc5Cl)CC4)ccc32)c1] | (3-(5-((4-(2- chlorobenzyl)piperazin-1- yl)methyl)-3-(4- (trifluoromethoxy)phenyl)- 1H-indol-1- yl)phenyl)methanamine |

In some embodiments, the compound of Formula (VIa) is selected from any one of the compounds from the following table:

TABLE 10B

| Compound | | |
|---|---|---|
| Ref. | Compound | Chemical Name |
| C206 | 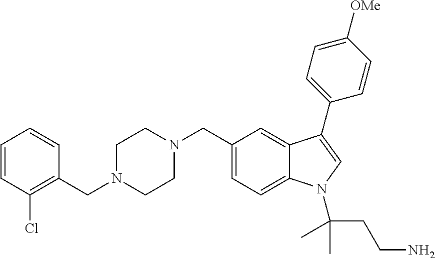 [SMILES: COc1ccc(-c2cn(C(C)(C)CCN)c3ccc(CN4CCN(Cc5ccccc5Cl)CC4)cc23)cc1] | 3-(5-((4-(2- chlorobenzyl)piperazin-1- yl)methyl)-3-(4- methoxyphenyl)-1H-indol- 1-yl)-3-methylbutan-1- amine |
| C212 | 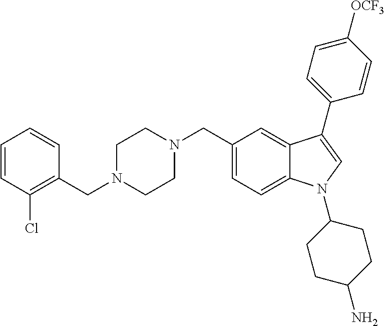 [SMILES: NC1CCC(n2cc(-c3ccc(OC(F)(F)F)cc3)c3cc(CN4CCN(Cc5ccccc5Cl)CC4)ccc32)CC1] | 4-(5-((4-(2- chlorobenzyl)piperazin-1- yl)methyl)-3-(4- (trifluoromethoxy)phenyl)- 1H-indol-1-yl)cyclohexan- 1-amine |

Also provided herein is a compound of Formula (VIIa), or a pharmaceutically acceptable salt, or solvate thereof:

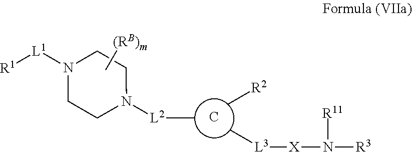

wherein,

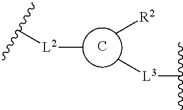

is a bicyclic heteroaryl that is selected from the following structures:

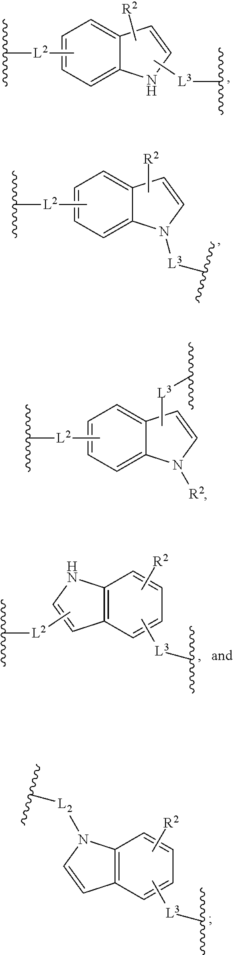

L¹ and L² are each independently absent, an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
each R^{B} is independently optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
L³ is an unsubstituted C₁-C₆alkylene;
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
R³ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, —C₁-C₄alkylene-(optionally substituted heteroaryl), —CH₂C(═O)R¹⁵, —C(═O)R¹⁵, or —CO₂R¹⁶;
R⁴ is hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹⁵ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁶ is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each m is independently 0, 1, 2, 3, or 4; and with the provision that the compound is not

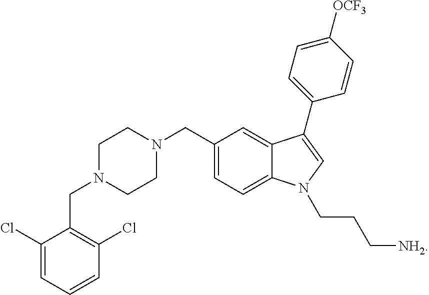 [SMILES: NCCCn1cc(-c2ccc(OC(F)(F)F)cc2)c2cc(CN3CCN(Cc4c(Cl)cccc4Cl)CC3)ccc21]

In some embodiments,

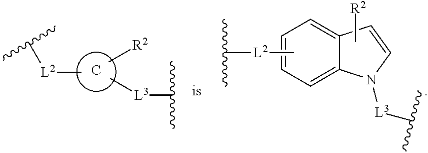

In some embodiments,

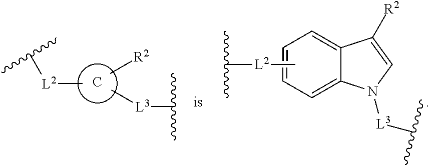

In some embodiments, the compound has the following structure of Formula (VIIb), or a pharmaceutically acceptable salt, or solvate thereof:

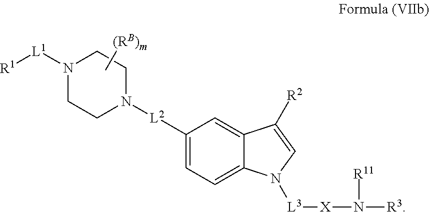

In some embodiments, the compound has the following structure of Formula (VIIc), or a pharmaceutically acceptable salt, or solvate thereof:

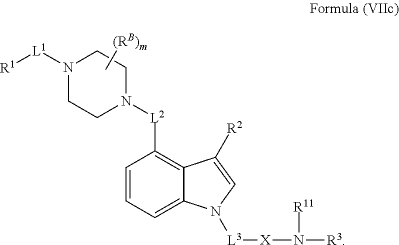

In some embodiments,

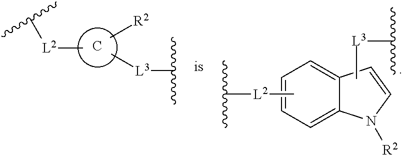

In some embodiments,

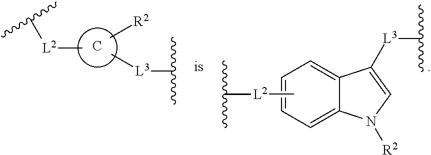

In some embodiments, the compound has the following structure of Formula (VIId), or a pharmaceutically acceptable salt, or solvate thereof:

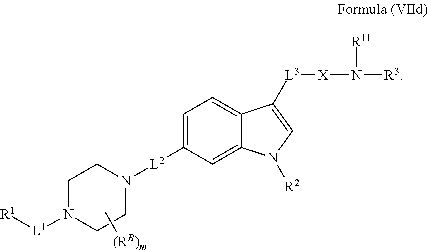

In some embodiments, the compound has the following structure of Formula (VIIe), or a pharmaceutically acceptable salt, or solvate thereof:

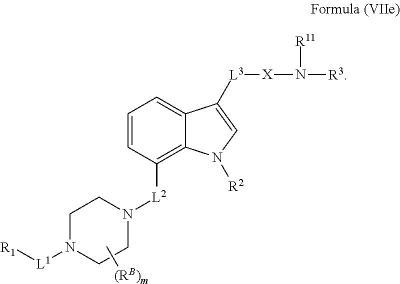

In some embodiments,

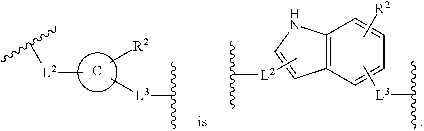

In some embodiments, L¹ is —CH₂—, C(═O), O, S, S(═O), S(═O)₂, or NR⁴. In some embodiments, L¹ is —CH₂—. In some embodiments, L² is —CH₂—, C(═O), O, S, S(═O), S(═O)₂, or NR⁴. In some embodiments, L² is —CH₂—. In some embodiments, L³ is —CH₂—, —CH₂CH₂—, or —CH₂—CH₂—CH₂—. In some embodiments, X is —CH₂— or C(═O). In some embodiments, X is —CH₂—.

In some embodiments, X is:

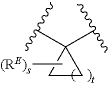

each s is independently 0, 1, 2, 3, or 4; and

t is 1, 2, 3, or 4.

In some embodiments, X is:

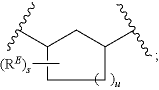

each s is independently 0, 1, 2, 3, or 4; and

u is 0, 1, or 2.

In some embodiments, L³-X is —CH₂—CH₂—CH₂—. In some embodiments, R¹¹ and R³ are each independently hydrogen, optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹¹ and R³ are each hydrogen. In some embodiments, R¹¹ and R³ are each optionally substituted C₁-C₆alkyl. In some embodiments, R¹¹ and R³ are each optionally substituted C₁-C₆heteroalkyl. In some embodiments, R¹⁵ and R¹⁶ are each independently optionally substituted C₁-C₆alkyl or optionally substituted C₁-C₆heteroalkyl. In some embodiments, R¹¹ is hydrogen and R³ is optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹ is an unsubstituted phenyl. In some embodiments, R¹ is a substituted phenyl. In some embodiments, R¹ is selected from:

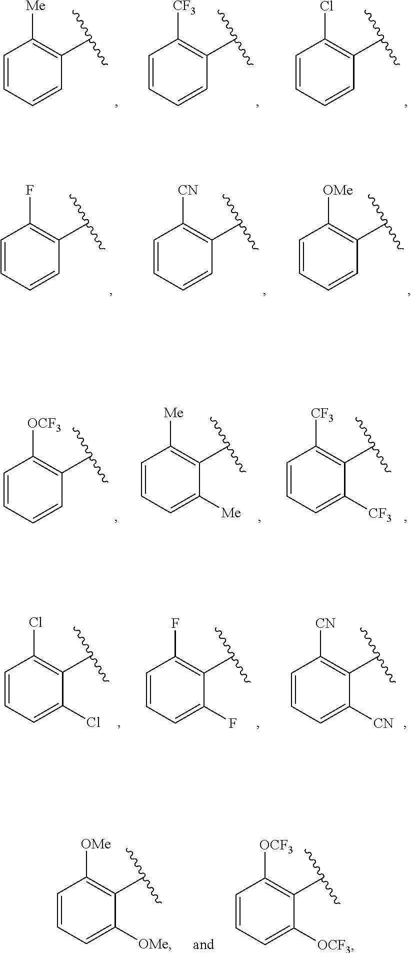

In some embodiments, R² is an unsubstituted phenyl. In some embodiments, R² is a substituted phenyl.

In some embodiments, R² is a substituted phenyl that is substituted with at least one —C(R^{x})₂—N(R^{y})₂, wherein each R^{x} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and each R^{y} is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or two R^{y} are taken together with the N atom to which they are attached to form an optionally substituted heterocycloalkyl ring. In some embodiments, each R^{x} is independently hydrogen. In some embodiments, each R^{y} is independently hydrogen.

In some embodiments, R² is selected from:

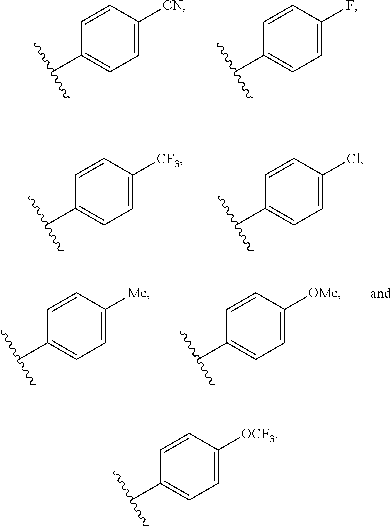

In some embodiments, R² is selected from:

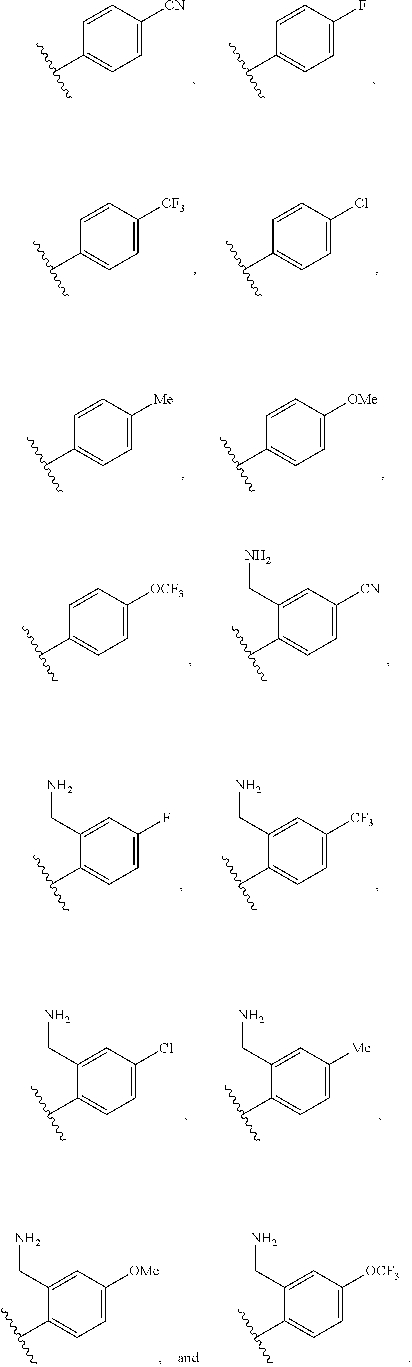

In some embodiments, R¹ is optionally substituted heterocycloalkyl. In some embodiments, R¹ is selected from:

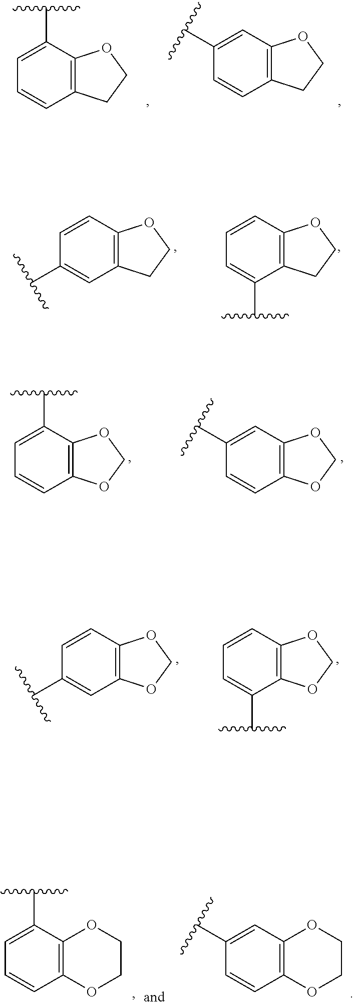

In some embodiments, R² is optionally substituted heterocycloalkyl. In some embodiments, R² is selected from:

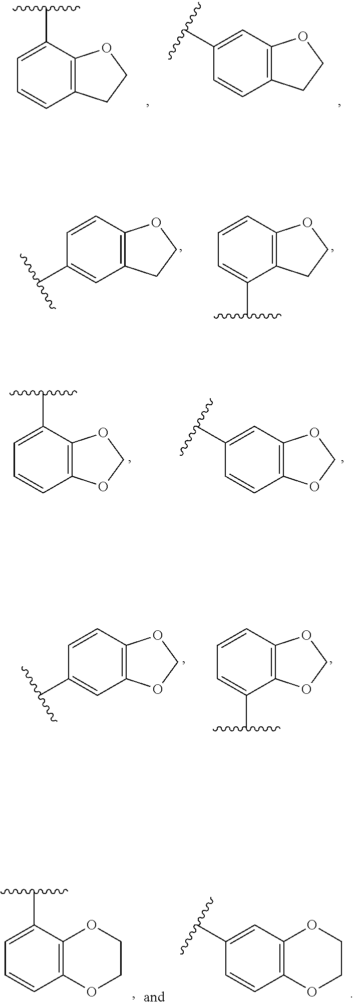

In some embodiments, the compound has the following structure of Formula (VIIf), or a pharmaceutically acceptable salt, or solvate thereof:

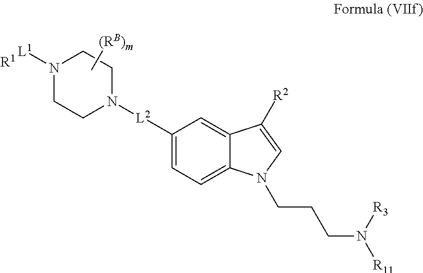

wherein R¹ and R² are each independently optionally substituted aryl.

In some embodiments, R₃ and R₁₁ are each hydrogen.

In some embodiments, the compound has the following structure of Formula (VIIg), or a pharmaceutically acceptable salt, or solvate thereof:

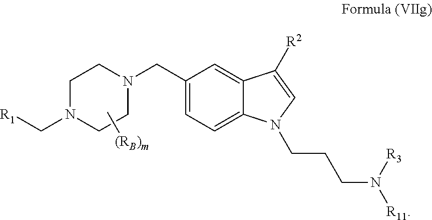

wherein R¹ and R² are each independently optionally substituted aryl.

In some embodiments, R₃ and R₁₁ are each hydrogen.

In some embodiments, the compound of Formula (VIIa) is any one of the compounds selected from:

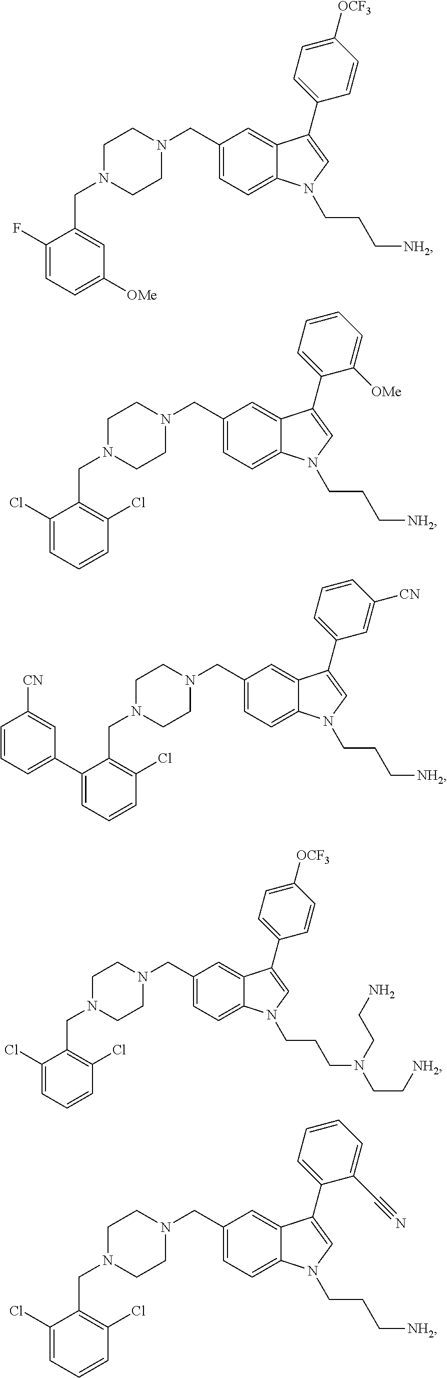

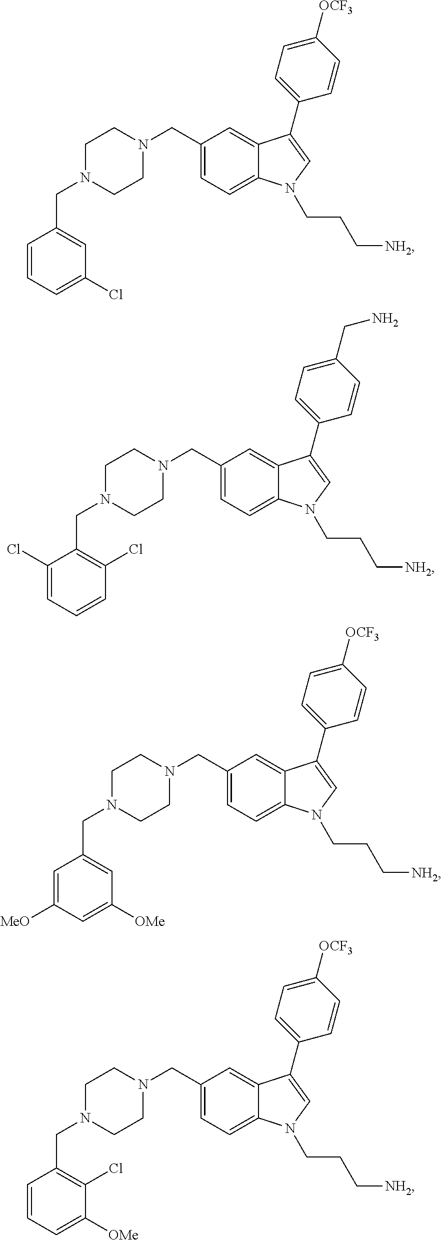 [SMILES: NCCCn1cc(-c2ccc(OC(F)(F)F)cc2)c2cc(CN3CCN(Cc4cccc(Cl)c4)CC3)ccc21 ; NCCCn1cc(-c2ccc(CN)cc2)c2cc(CN3CCN(Cc4c(Cl)cccc4Cl)CC3)ccc21 ; COc1cc(CN2CCN(Cc3ccc4c(c3)c(-c3ccc(OC(F)(F)F)cc3)cn4CCCN)CC2)cc(OC)c1 ; COc1cccc(CN2CCN(Cc3ccc4c(c3)c(-c3ccc(OC(F)(F)F)cc3)cn4CCCN)CC2)c1Cl]

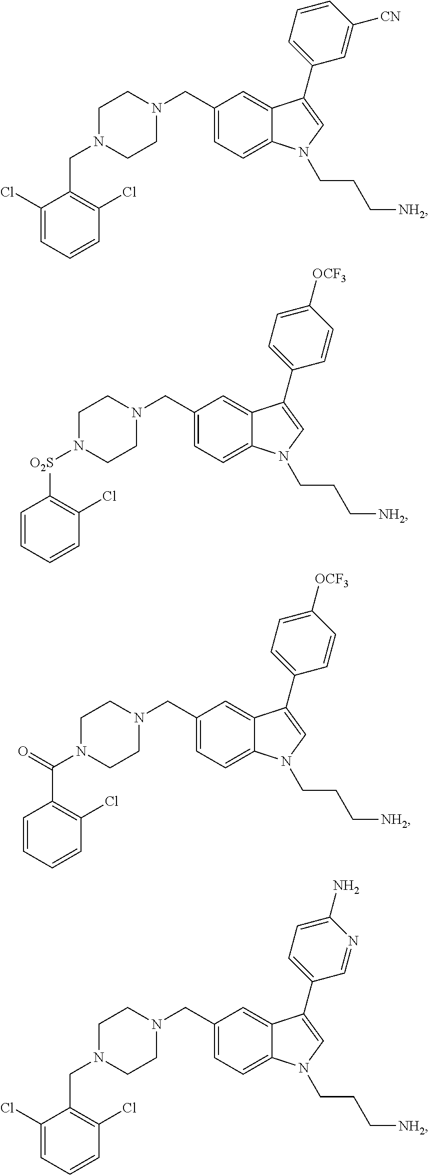 [SMILES: N#Cc1cccc(-c2cn(CCCN)c3ccc(CN4CCN(Cc5c(Cl)cccc5Cl)CC4)cc23)c1 ; NCCCn1cc(-c2ccc(OC(F)(F)F)cc2)c2cc(CN3CCN(S(=O)(=O)c4ccccc4Cl)CC3)ccc21 ; NCCCn1cc(-c2ccc(OC(F)(F)F)cc2)c2cc(CN3CCN(C(=O)c4ccccc4Cl)CC3)ccc21 ; NCCCn1cc(-c2ccc(N)nc2)c2cc(CN3CCN(Cc4c(Cl)cccc4Cl)CC3)ccc21]

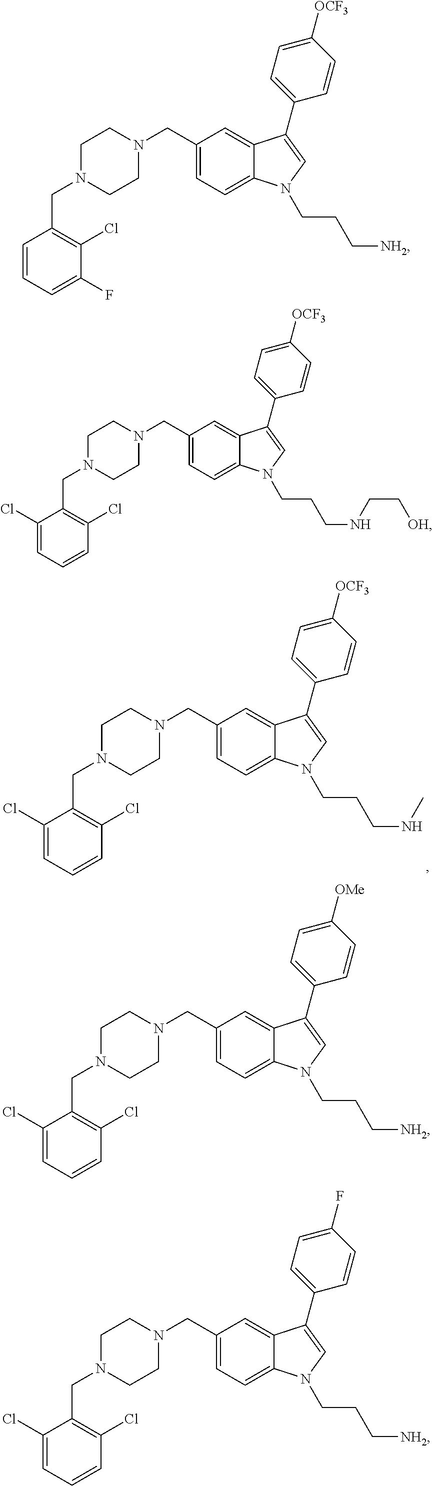 [SMILES: NCCCn1cc(-c2ccc(OC(F)(F)F)cc2)c2cc(CN3CCN(Cc4cccc(F)c4Cl)CC3)ccc21 ; OCCNCCCn1cc(-c2ccc(OC(F)(F)F)cc2)c2cc(CN3CCN(Cc4c(Cl)cccc4Cl)CC3)ccc21 ; CNCCCn1cc(-c2ccc(OC(F)(F)F)cc2)c2cc(CN3CCN(Cc4c(Cl)cccc4Cl)CC3)ccc21 ; COc1ccc(-c2cn(CCCN)c3ccc(CN4CCN(Cc5c(Cl)cccc5Cl)CC4)cc23)cc1 ; NCCCn1cc(-c2ccc(F)cc2)c2cc(CN3CCN(Cc4c(Cl)cccc4Cl)CC3)ccc21]

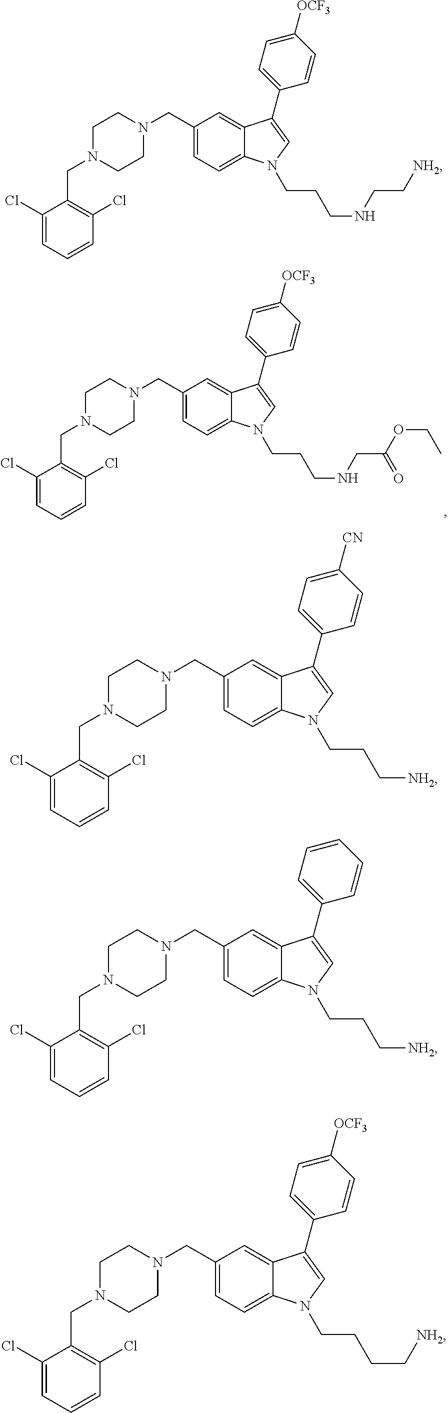 [SMILES: NCCNCCCn1cc(-c2ccc(OC(F)(F)F)cc2)c2cc(CN3CCN(Cc4c(Cl)cccc4Cl)CC3)ccc21 ; CCOC(=O)CNCCCn1cc(-c2ccc(OC(F)(F)F)cc2)c2cc(CN3CCN(Cc4c(Cl)cccc4Cl)CC3)ccc21 ; N#Cc1ccc(-c2cn(CCCN)c3ccc(CN4CCN(Cc5c(Cl)cccc5Cl)CC4)cc23)cc1 ; NCCCn1cc(-c2ccccc2)c2cc(CN3CCN(Cc4c(Cl)cccc4Cl)CC3)ccc21 ; NCCCCn1cc(-c2ccc(OC(F)(F)F)cc2)c2cc(CN3CCN(Cc4c(Cl)cccc4Cl)CC3)ccc21]

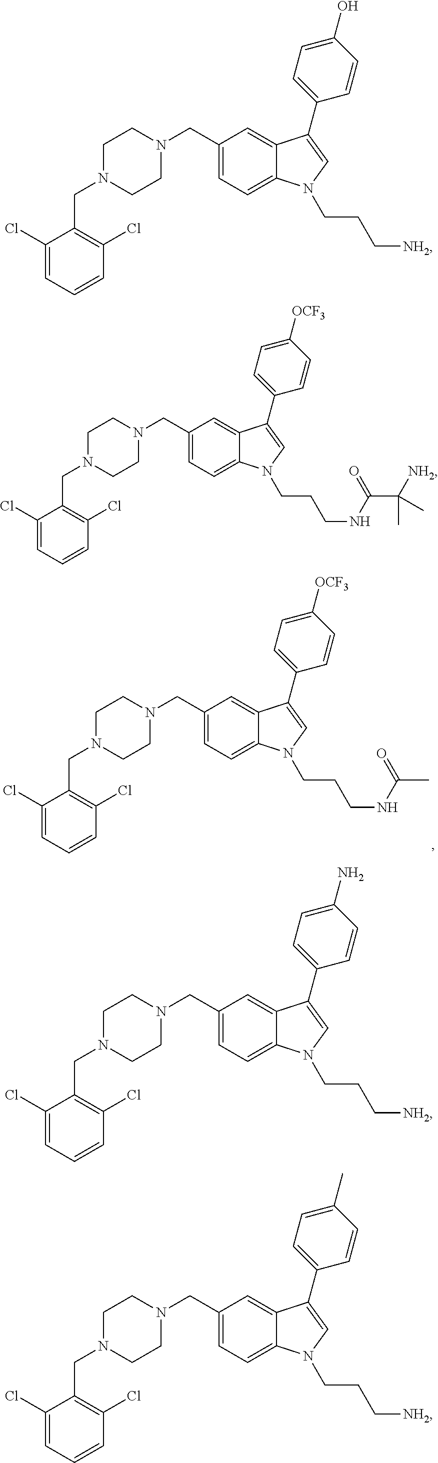 [SMILES: NCCCn1cc(-c2ccc(O)cc2)c2cc(CN3CCN(Cc4c(Cl)cccc4Cl)CC3)ccc21 ; CC(C)(N)C(=O)NCCCn1cc(-c2ccc(OC(F)(F)F)cc2)c2cc(CN3CCN(Cc4c(Cl)cccc4Cl)CC3)ccc21 ; CC(=O)NCCCn1cc(-c2ccc(OC(F)(F)F)cc2)c2cc(CN3CCN(Cc4c(Cl)cccc4Cl)CC3)ccc21 ; NCCCn1cc(-c2ccc(N)cc2)c2cc(CN3CCN(Cc4c(Cl)cccc4Cl)CC3)ccc21 ; Cc1ccc(-c2cn(CCCN)c3ccc(CN4CCN(Cc5c(Cl)cccc5Cl)CC4)cc23)cc1]

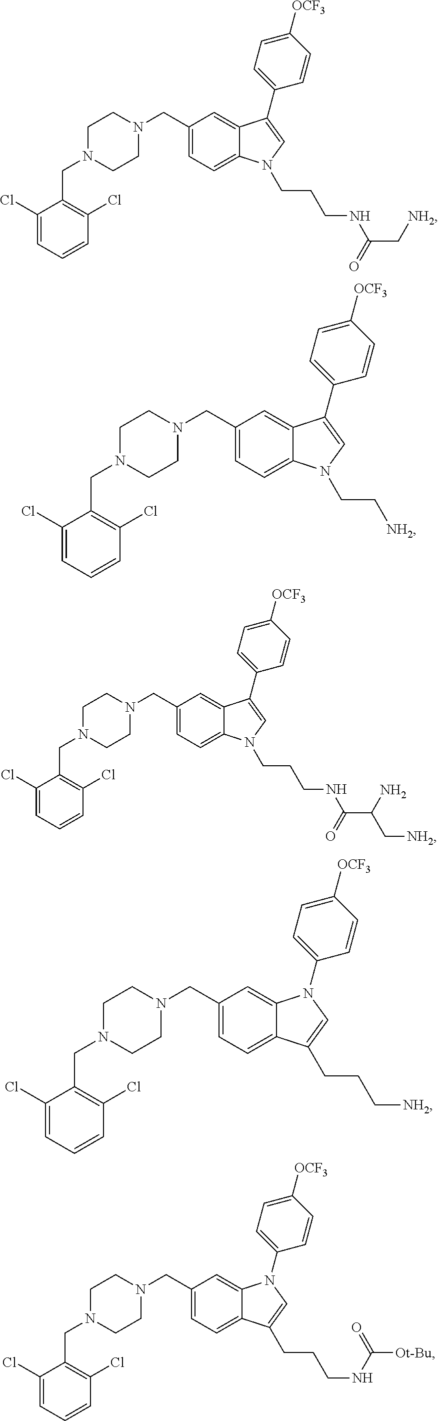

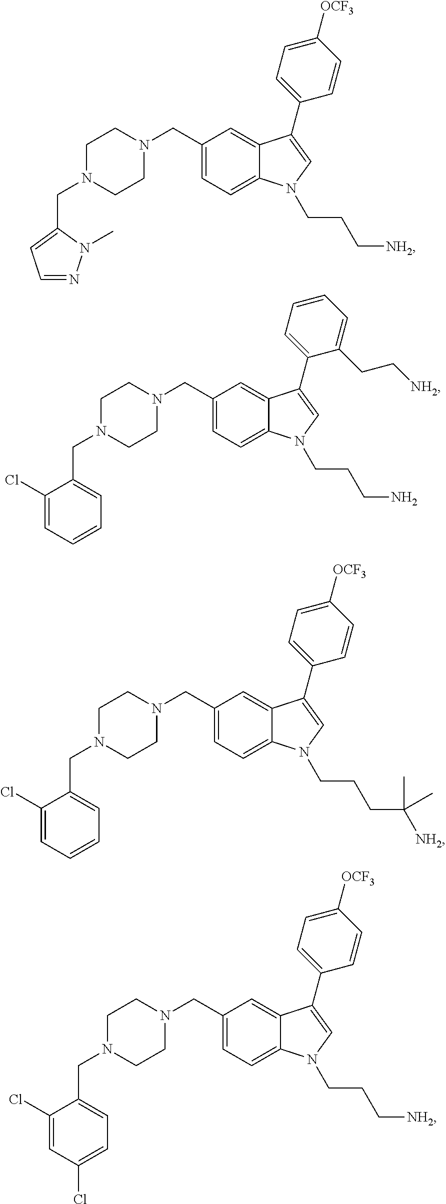 [SMILES: Cn1nccc1CN1CCN(Cc2ccc3c(c2)c(-c2ccc(OC(F)(F)F)cc2)cn3CCCN)CC1 ; NCCCn1cc(-c2ccccc2CCN)c2cc(CN3CCN(Cc4ccccc4Cl)CC3)ccc21 ; CC(C)(N)CCCn1cc(-c2ccc(OC(F)(F)F)cc2)c2cc(CN3CCN(Cc4ccccc4Cl)CC3)ccc21 ; NCCCn1cc(-c2ccc(OC(F)(F)F)cc2)c2cc(CN3CCN(Cc4ccc(Cl)cc4Cl)CC3)ccc21]

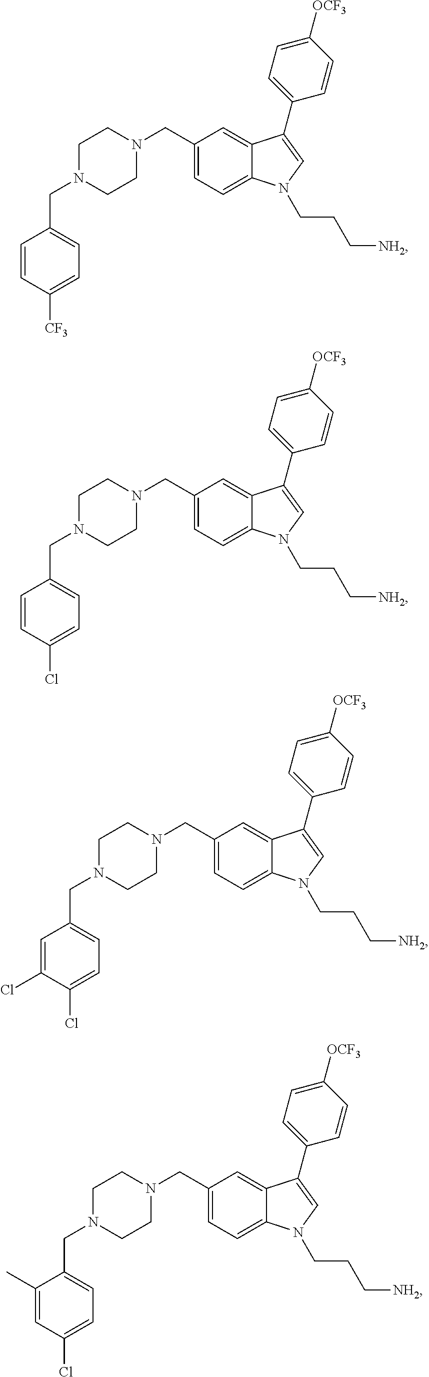 [SMILES: NCCCn1cc(-c2ccc(OC(F)(F)F)cc2)c2cc(CN3CCN(Cc4ccc(C(F)(F)F)cc4)CC3)ccc21 ; NCCCn1cc(-c2ccc(OC(F)(F)F)cc2)c2cc(CN3CCN(Cc4ccc(Cl)cc4)CC3)ccc21 ; NCCCn1cc(-c2ccc(OC(F)(F)F)cc2)c2cc(CN3CCN(Cc4ccc(Cl)c(Cl)c4)CC3)ccc21 ; Cc1cc(Cl)ccc1CN1CCN(Cc2ccc3c(c2)c(-c2ccc(OC(F)(F)F)cc2)cn3CCCN)CC1]

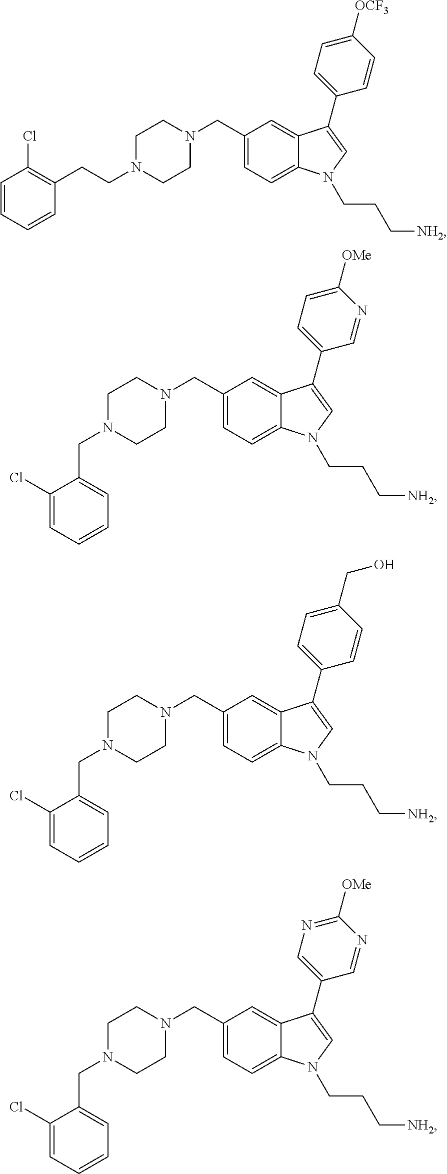 [SMILES: NCCCn1cc(-c2ccc(OC(F)(F)F)cc2)c2cc(CN3CCN(CCc4ccccc4Cl)CC3)ccc21 ; COc1ccc(-c2cn(CCCN)c3ccc(CN4CCN(Cc5ccccc5Cl)CC4)cc23)cn1 ; NCCCn1cc(-c2ccc(CO)cc2)c2cc(CN3CCN(Cc4ccccc4Cl)CC3)ccc21 ; COc1ncc(-c2cn(CCCN)c3ccc(CN4CCN(Cc5ccccc5Cl)CC4)cc23)cn1]

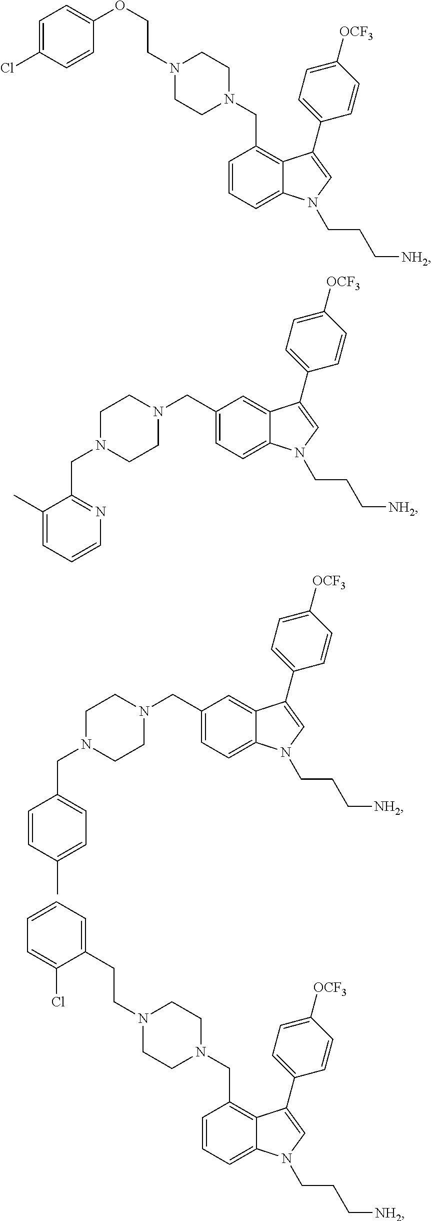

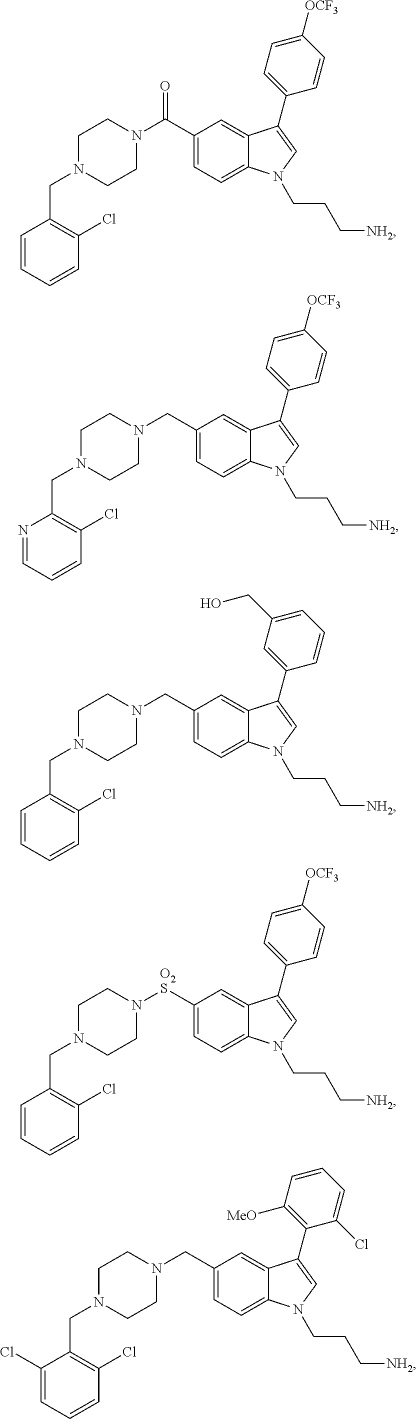 [SMILES: NCCCn1cc(-c2ccc(OC(F)(F)F)cc2)c2cc(C(=O)N3CCN(Cc4ccccc4Cl)CC3)ccc21 ; NCCCn1cc(-c2ccc(OC(F)(F)F)cc2)c2cc(CN3CCN(Cc4ncccc4Cl)CC3)ccc21 ; NCCCn1cc(-c2cccc(CO)c2)c2cc(CN3CCN(Cc4ccccc4Cl)CC3)ccc21 ; NCCCn1cc(-c2ccc(OC(F)(F)F)cc2)c2cc(S(=O)(=O)N3CCN(Cc4ccccc4Cl)CC3)ccc21 ; COc1cccc(Cl)c1-c1cn(CCCN)c2ccc(CN3CCN(Cc4c(Cl)cccc4Cl)CC3)cc12]

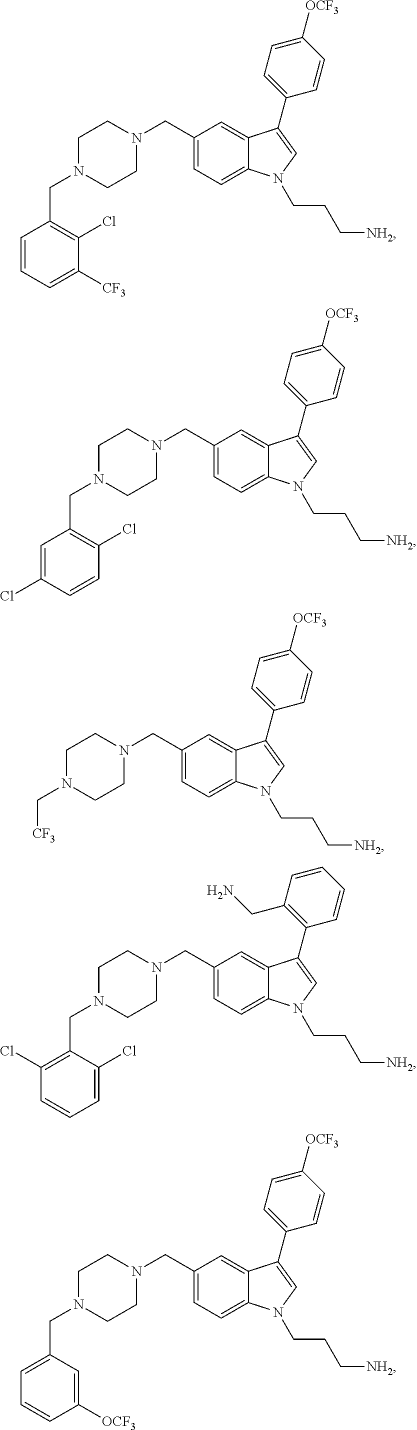

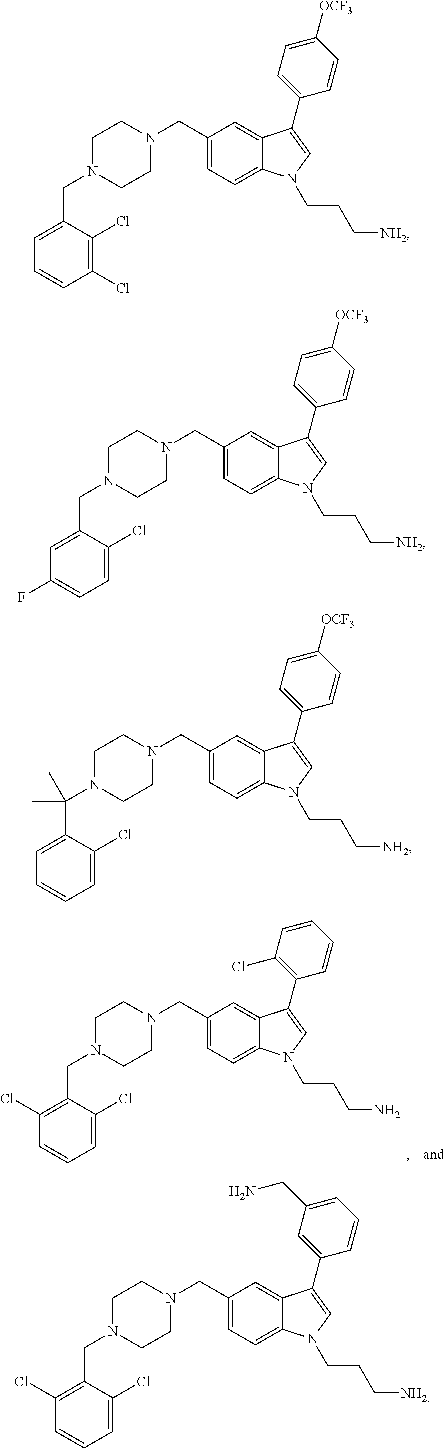 [SMILES: NCCCn1cc(-c2ccc(OC(F)(F)F)cc2)c2cc(CN3CCN(Cc4cccc(Cl)c4Cl)CC3)ccc21 ; NCCCn1cc(-c2ccc(OC(F)(F)F)cc2)c2cc(CN3CCN(Cc4cc(F)ccc4Cl)CC3)ccc21 ; CC(C)(c1ccccc1Cl)N1CCN(Cc2ccc3c(c2)c(-c2ccc(OC(F)(F)F)cc2)cn3CCCN)CC1 ; NCCCn1cc(-c2ccccc2Cl)c2cc(CN3CCN(Cc4c(Cl)cccc4Cl)CC3)ccc21 ; NCCCn1cc(-c2cccc(CN)c2)c2cc(CN3CCN(Cc4c(Cl)cccc4Cl)CC3)ccc21]

In some embodiments, the compound of Formula (VIIa) is any one of the compounds selected from:

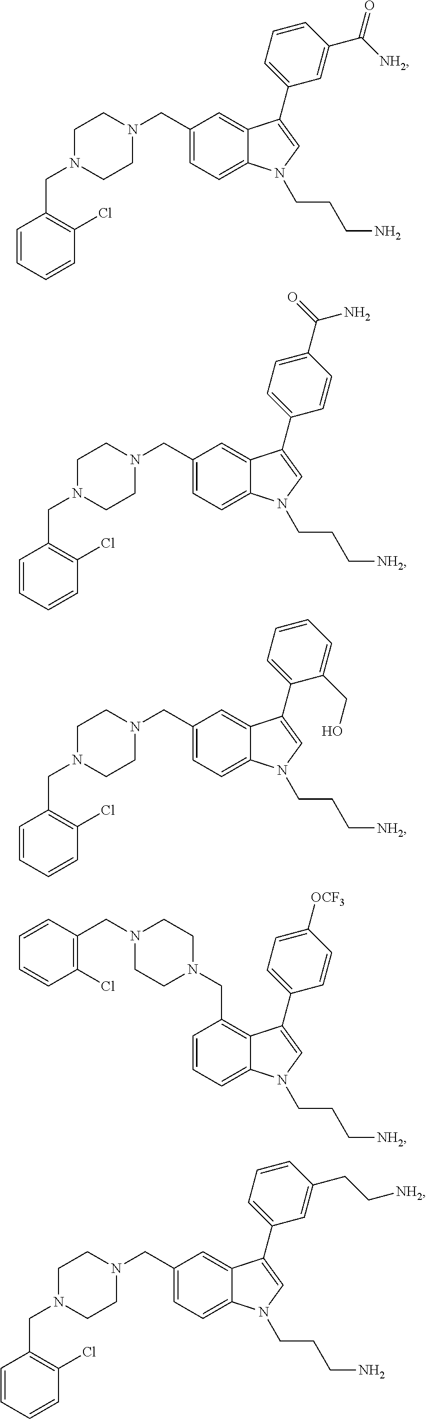

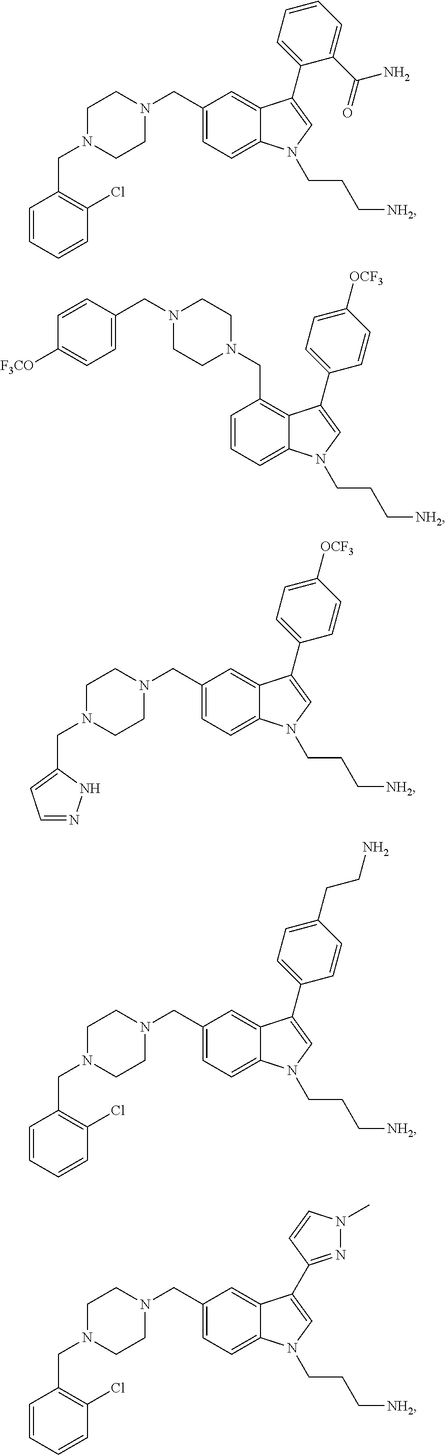 [SMILES: NCCCn1cc(-c2ccccc2C(N)=O)c2cc(CN3CCN(Cc4ccccc4Cl)CC3)ccc21 ; NCCCn1cc(-c2ccc(OC(F)(F)F)cc2)c2c(CN3CCN(Cc4ccc(OC(F)(F)F)cc4)CC3)cccc21 ; NCCCn1cc(-c2ccc(OC(F)(F)F)cc2)c2cc(CN3CCN(Cc4ccn[nH]4)CC3)ccc21 ; NCCCn1cc(-c2ccc(CCN)cc2)c2cc(CN3CCN(Cc4ccccc4Cl)CC3)ccc21 ; Cn1ccc(-c2cn(CCCN)c3ccc(CN4CCN(Cc5ccccc5Cl)CC4)cc23)n1]

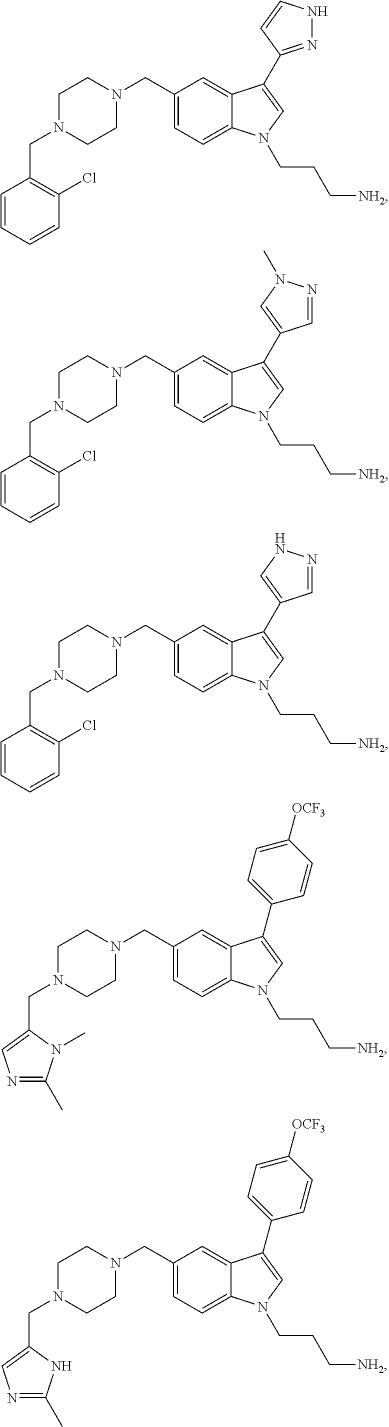 [SMILES: NCCCn1cc(-c2cc[nH]n2)c2cc(CN3CCN(Cc4ccccc4Cl)CC3)ccc21 ; Cn1cc(-c2cn(CCCN)c3ccc(CN4CCN(Cc5ccccc5Cl)CC4)cc23)cn1 ; NCCCn1cc(-c2cn[nH]c2)c2cc(CN3CCN(Cc4ccccc4Cl)CC3)ccc21 ; Cc1ncc(CN2CCN(Cc3ccc4c(c3)c(-c3ccc(OC(F)(F)F)cc3)cn4CCCN)CC2)n1C ; Cc1ncc(CN2CCN(Cc3ccc4c(c3)c(-c3ccc(OC(F)(F)F)cc3)cn4CCCN)CC2)[nH]1]

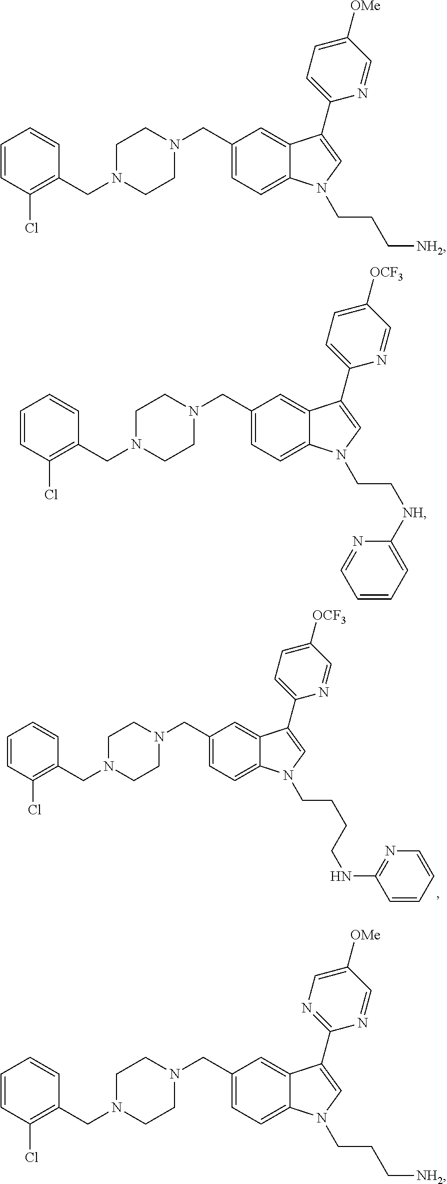

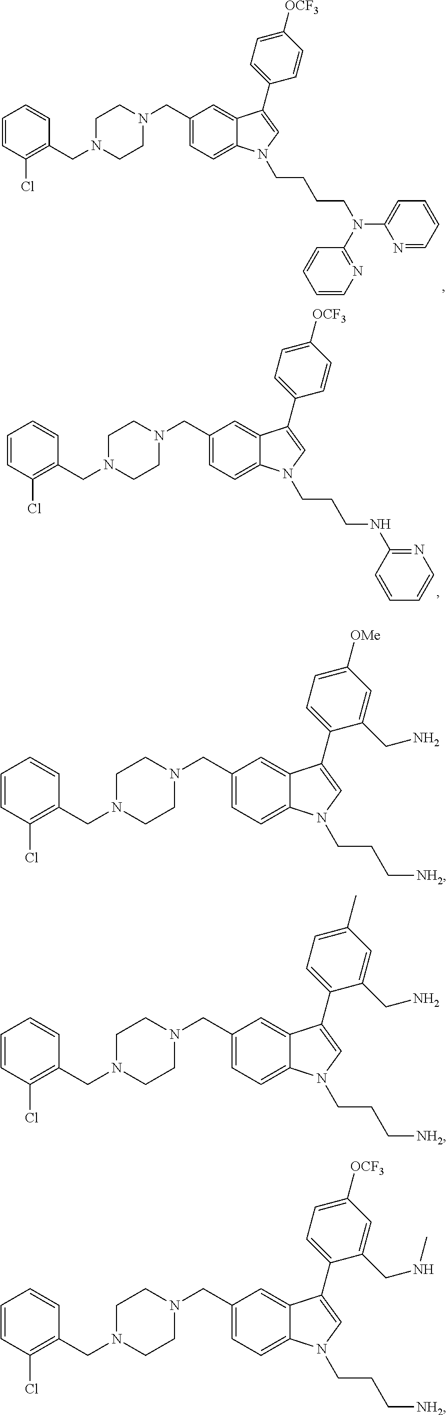

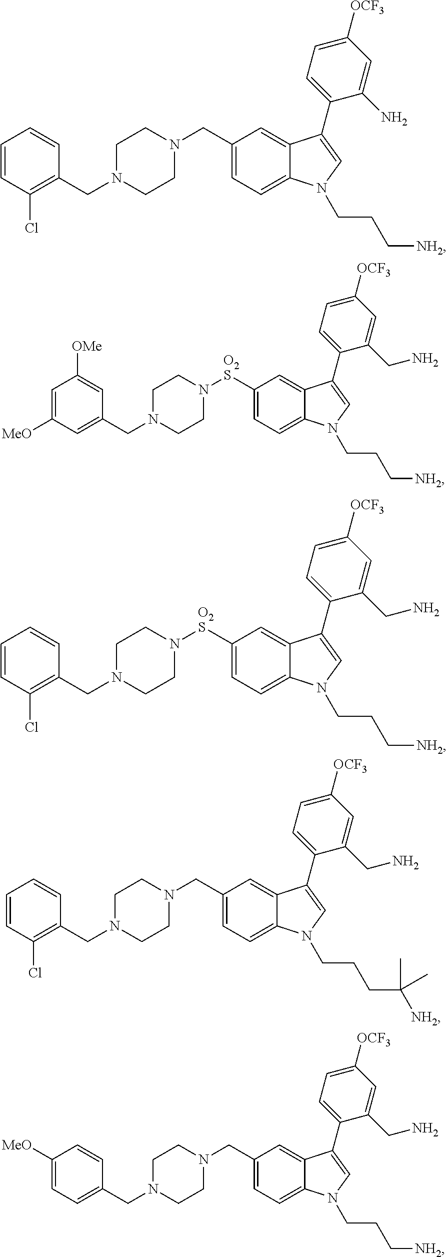

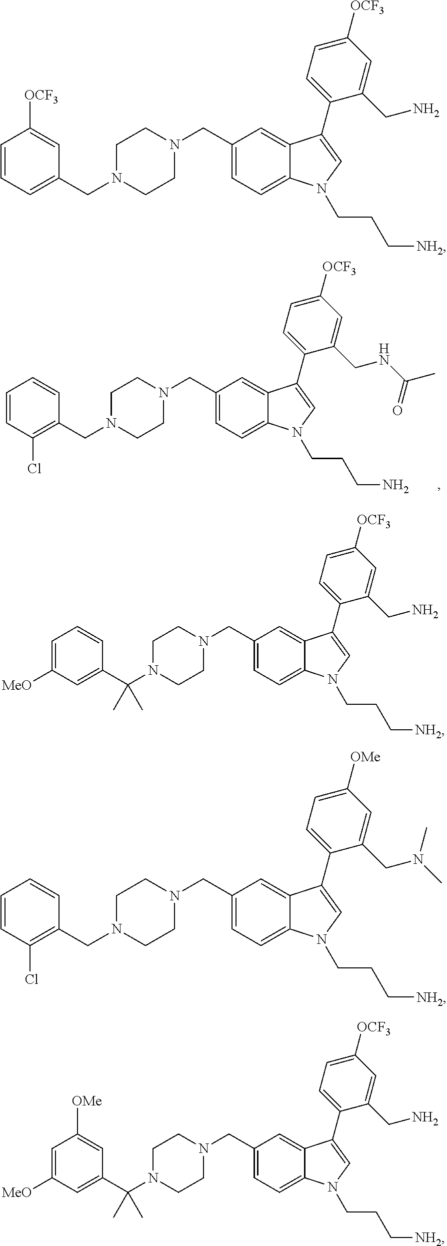

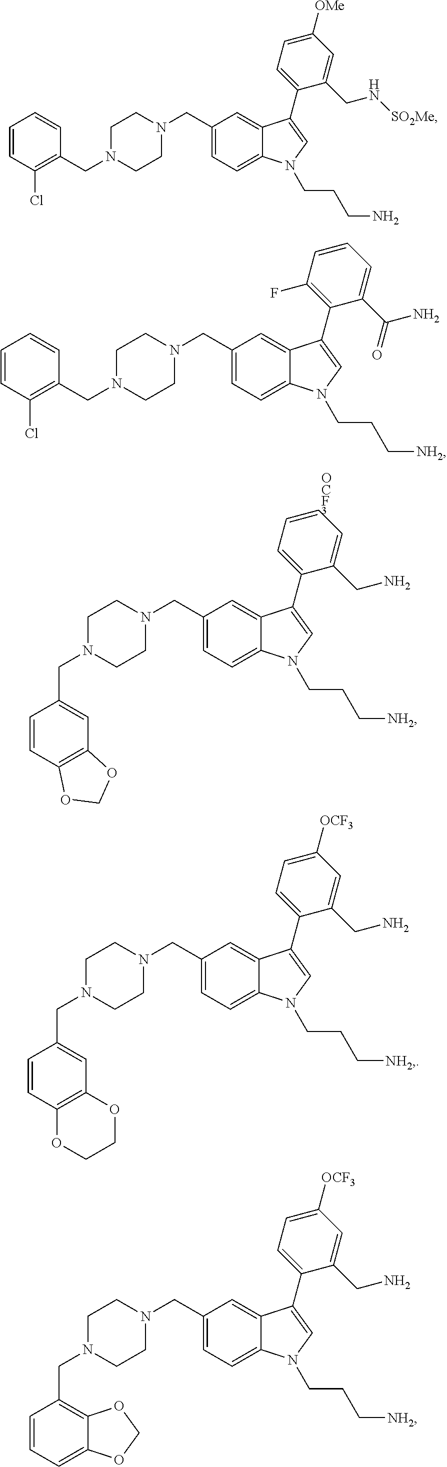

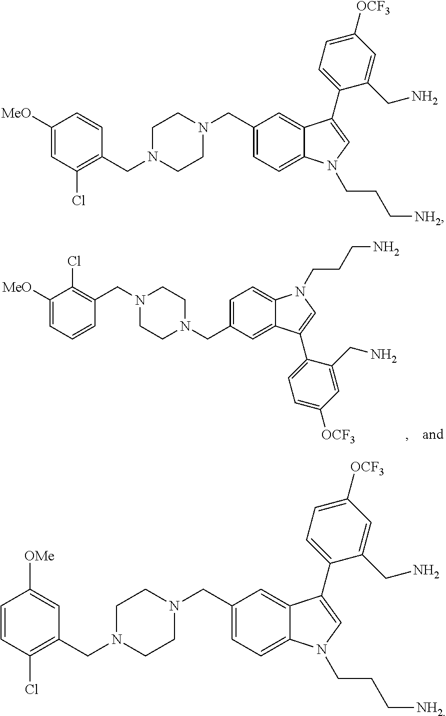

In some embodiments, the compound of Formula (VIIa) is selected from any one of the compounds from the following table:

TABLE 11A

| Compound | | |
|---|---|---|
| Ref. | Compound | Chemical Name |
| C1 | 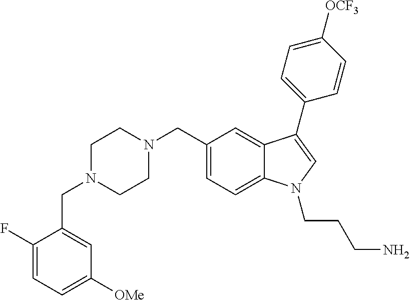 [SMILES: COc1ccc(F)c(CN2CCN(Cc3ccc4c(c3)c(-c3ccc(OC(F)(F)F)cc3)cn4CCCN)CC2)c1] | 3-(5-((4-(2-fluoro-5- methoxybenzyl)piperazin- 1-yl)methyl)-3-(4- (trifluoromethoxy)phenyl)- 1H-indol-1-yl)propan- 1-amine |
| C2 | 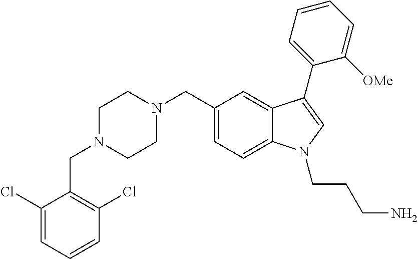 [SMILES: COc1ccccc1-c1cn(CCCN)c2ccc(CN3CCN(Cc4c(Cl)cccc4Cl)CC3)cc12] | 3-(5-((4-(2,6- dichlorobenzyl)piperazin- 1-yl)methyl)-3-(2- methoxyphenyl)-1H- indol-1-yl)propan-1- amine |
| C4 | 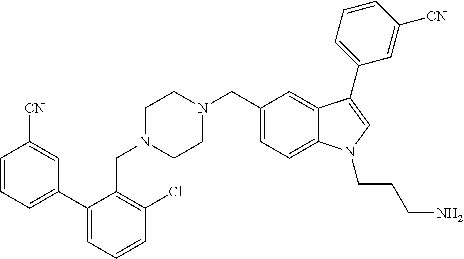 [SMILES: N#Cc1cccc(-c2cccc(Cl)c2CN2CCN(Cc3ccc4c(c3)c(-c3cccc(C#N)c3)cn4CCCN)CC2)c1] | 2′-((4-((1-(3- aminopropyl)-3-(3- cyanophenyl)-1H-indol- 5-yl)methyl)piperazin-1- yl)methyl)-3′- chlorobiphenyl-3- carbonitrile |
| C7 | 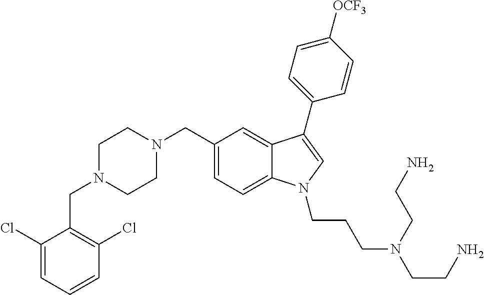 [SMILES: NCCN(CCN)CCCn1cc(-c2ccc(OC(F)(F)F)cc2)c2cc(CN3CCN(Cc4c(Cl)cccc4Cl)CC3)ccc21] | N1-(2-aminoethyl)-N1- (3-(5-((4-(2,6- dichlorobenzyl)piperazin- 1-yl)methyl)-3-(4- (trifluoromethoxy)phenyl)- 1H-indol-1- yl)propyl)ethane-1,2- diamine |
| C8 | 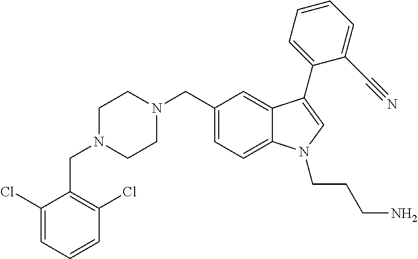 [SMILES: N#Cc1ccccc1-c1cn(CCCN)c2ccc(CN3CCN(Cc4c(Cl)cccc4Cl)CC3)cc12] | 2-(1-(3-aminopropyl)-5- ((4-(2,6- dichlorobenzyl)piperazin- 1-yl)methyl)-1H-indol- 3-yl)benzonitrile |
| C9 | 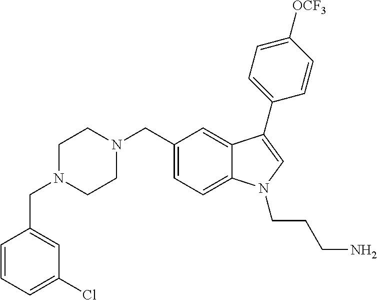 [SMILES: NCCCn1cc(-c2ccc(OC(F)(F)F)cc2)c2cc(CN3CCN(Cc4cccc(Cl)c4)CC3)ccc21] | 3-(5-((4-(3- chlorobenzyl)piperazin- 1-yl)methyl)-3-(4- (trifluoromethoxy)phenyl)- 1H-indol-1-yl)propan- 1-amine |
| C10 | 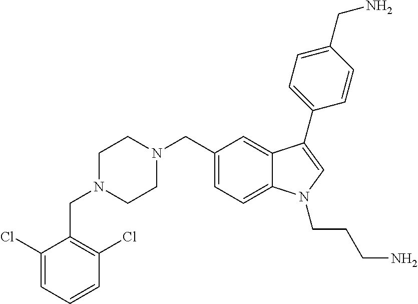 [SMILES: NCCCn1cc(-c2ccc(CN)cc2)c2cc(CN3CCN(Cc4c(Cl)cccc4Cl)CC3)ccc21] | 3-(3-(4- (aminomethyl)phenyl)-5- ((4-(2,6- dichlorobenzyl)piperazin- 1-yl)methyl)-1H-indol- 1-yl)propan-1-amine |
| C11 | 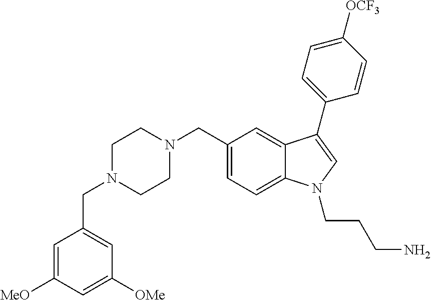 [SMILES: COc1cc(CN2CCN(Cc3ccc4c(c3)c(-c3ccc(OC(F)(F)F)cc3)cn4CCCN)CC2)cc(OC)c1] | 3-(5-((4-(3,5- dimethoxybenzyl)piperazin- 1-yl)methyl)-3-(4- (trifluoromethoxy)phenyl)- 1H-indol-1-yl)propan- 1-amine |
| C12 | 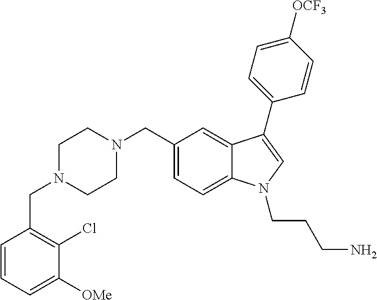 [SMILES: COc1cccc(CN2CCN(Cc3ccc4c(c3)c(-c3ccc(OC(F)(F)F)cc3)cn4CCCN)CC2)c1Cl] | 3-(5-((4-(2-chloro-3- methoxybenzyl)piperazin- 1-yl)methyl)-3-(4- (trifluoromethoxy)phenyl)- 1H-indol-1-yl)propan- 1-amine |
| C13 | 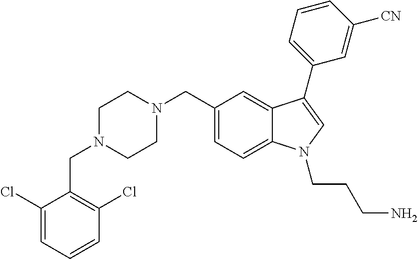 [SMILES: N#Cc1cccc(-c2cn(CCCN)c3ccc(CN4CCN(Cc5c(Cl)cccc5Cl)CC4)cc23)c1] | 3-(1-(3-aminopropyl)-5- ((4-(2,6- dichlorobenzyl)piperazin- 1-yl)methyl)-1H-indol- 3-yl)benzonitrile |
| C14 | 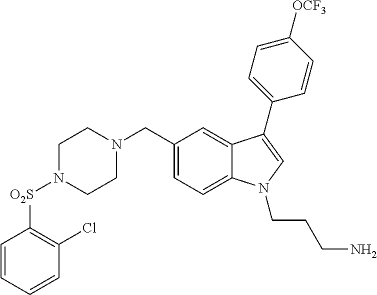 [SMILES: NCCCn1cc(-c2ccc(OC(F)(F)F)cc2)c2cc(CN3CCN(S(=O)(=O)c4ccccc4Cl)CC3)ccc21] | 3-(5-((4-(2- chlorophenylsulfonyl) piperazin-1-yl)methyl)-3-(4- (trifluoromethoxy)phenyl)- 1H-indol-1-yl)propan- 1-amine |
| C15 | 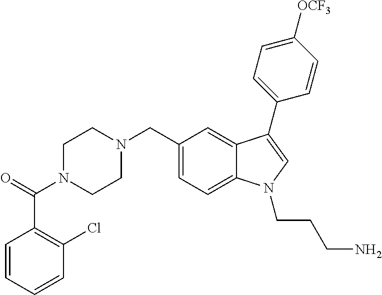 [SMILES: NCCCn1cc(-c2ccc(OC(F)(F)F)cc2)c2cc(CN3CCN(C(=O)c4ccccc4Cl)CC3)ccc21] | (4-((1-(3-aminopropyl)- 3-(4- (trifluoromethoxy)phenyl)- 1H-indol-5- yl)methyl)piperazin-1- yl)(2- chlorophenyl)methanone |
| C18 | 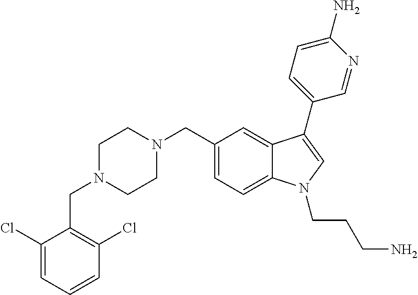 [SMILES: NCCCn1cc(-c2ccc(N)nc2)c2cc(CN3CCN(Cc4c(Cl)cccc4Cl)CC3)ccc21] | 5-(1-(3-aminopropyl)-5- ((4-(2,6- dichlorobenzyl)piperazin- 1-yl)methyl)-1H-indol- 3-yl)pyridin-2-amine |
| C19 | 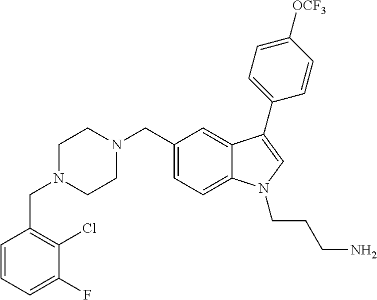 [SMILES: NCCCn1cc(-c2ccc(OC(F)(F)F)cc2)c2cc(CN3CCN(Cc4cccc(F)c4Cl)CC3)ccc21] | 3-(5-((4-(2-chloro-3- fluorobenzyl)piperazin-1- yl)methyl)-3-(4- (trifluoromethoxy)phenyl)- 1H-indol-1-yl)propan- 1-amine |
| C20 | 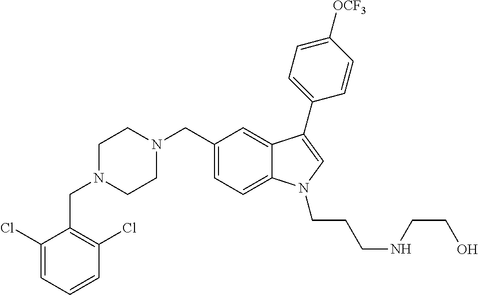 [SMILES: OCCNCCCn1cc(-c2ccc(OC(F)(F)F)cc2)c2cc(CN3CCN(Cc4c(Cl)cccc4Cl)CC3)ccc21] | 2-(3-(5-((4-(2,6- dichlorobenzyl)piperazin- 1-yl)methyl)-3-(4- (trifluoromethoxy)phenyl)- 1H-indol-1- yl)propylamino)ethanol |
| C21 | 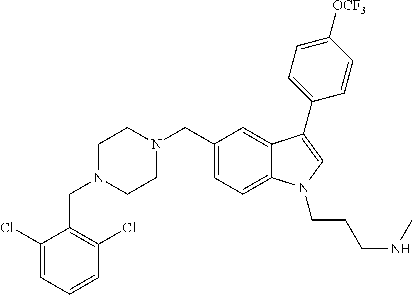 [SMILES: CNCCCn1cc(-c2ccc(OC(F)(F)F)cc2)c2cc(CN3CCN(Cc4c(Cl)cccc4Cl)CC3)ccc21] | 3-(5-((4-(2,6- dichlorobenzyl)piperazin- 1-yl)methyl)-3-(4- (trifluoromethoxy)phenyl)- 1H-indol-1-yl)-N- methylpropan-1-amine |
| C22 | 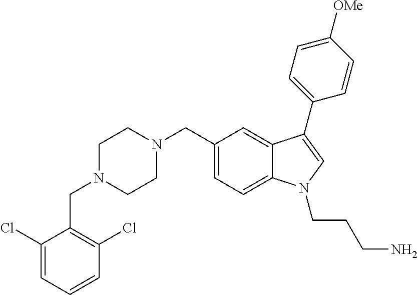 [SMILES: COc1ccc(-c2cn(CCCN)c3ccc(CN4CCN(Cc5c(Cl)cccc5Cl)CC4)cc23)cc1] | 3-(5-((4-(2,6- dichlorobenzyl)piperazin- 1-yl)methyl)-3-(4- methoxyphenyl)-1H- indol-1-yl)propan-1- amine |
| C23 | 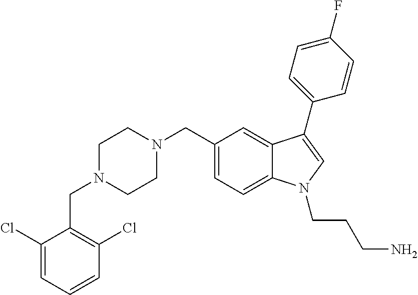 [SMILES: NCCCn1cc(-c2ccc(F)cc2)c2cc(CN3CCN(Cc4c(Cl)cccc4Cl)CC3)ccc21] | 3-(5-((4-(2,6- dichlorobenzyl)piperazin- 1-yl)methyl)-3-(4- fluorophenyl)-1H-indol- 1-yl)propan-1-amine |
| C26 | 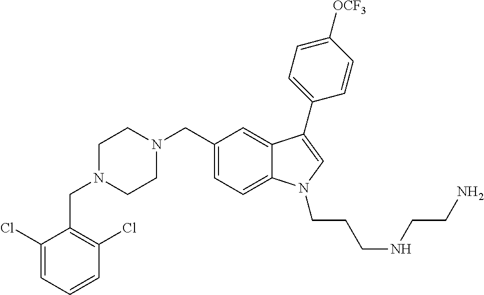 [SMILES: NCCNCCCn1cc(-c2ccc(OC(F)(F)F)cc2)c2cc(CN3CCN(Cc4c(Cl)cccc4Cl)CC3)ccc21] | N1-(3-(5-((4-(2,6- dichlorobenzyl)piperazin- 1-yl)methyl)-3-(4- (trifluoromethoxy)phenyl)- 1-H-indol-1- yl)propyl)ethane-1,2- diamine |
| C28 | 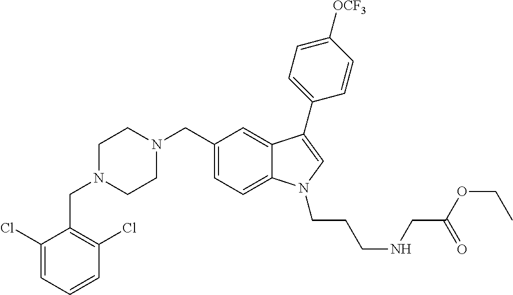 [SMILES: CCOC(=O)CNCCCn1cc(-c2ccc(OC(F)(F)F)cc2)c2cc(CN3CCN(Cc4c(Cl)cccc4Cl)CC3)ccc21] | ethyl 2-(3-(5-((4-(2,6- dichlorobenzyl)piperazin- 1-yl)methyl)-3-(4- (trifluoromethoxy)phenyl)- 1H-indol-1- yl)propylamino)acetate |
| C33 | 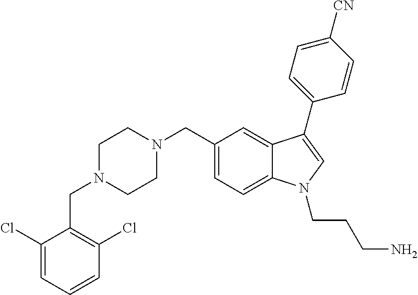 [SMILES: N#Cc1ccc(-c2cn(CCCN)c3ccc(CN4CCN(Cc5c(Cl)cccc5Cl)CC4)cc23)cc1] | 4-(1-(3-aminopropyl)-5- ((4-(2,6- dichlorobenzyl)piperazin- 1-yl)methyl)-1H-indol- 3-yl)benzonitrile |
| C37 | 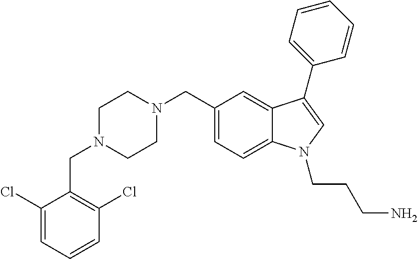 [SMILES: NCCCn1cc(-c2ccccc2)c2cc(CN3CCN(Cc4c(Cl)cccc4Cl)CC3)ccc21] | 3-(5-((4-(2,6- dichlorobenzyl)piperazin- 1-yl)methyl)-3-phenyl- 1H-indol-1-yl)propan-1- amine |
| C43 | 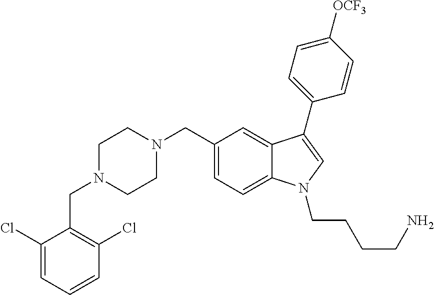 [SMILES: NCCCCn1cc(-c2ccc(OC(F)(F)F)cc2)c2cc(CN3CCN(Cc4c(Cl)cccc4Cl)CC3)ccc21] | 4-(5-((4-(2,6- dichlorobenzyl)piperazin- 1-yl)methyl)-3-(4- (trifluoromethoxy)phenyl)- 1H-indol-1-yl)butan-1- amine |
| C44 | 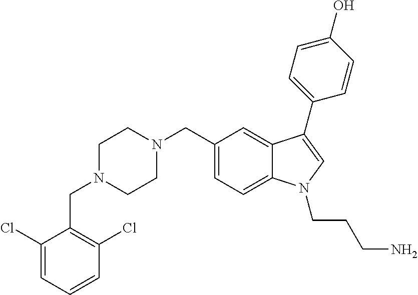 [SMILES: NCCCn1cc(-c2ccc(O)cc2)c2cc(CN3CCN(Cc4c(Cl)cccc4Cl)CC3)ccc21] | 4-(1-(3-aminopropyl)-5- ((4-(2,6- dichlorobenzyl)piperazin- 1-yl)methyl)-1H-indol- 3-yl)phenol |
| C46 | 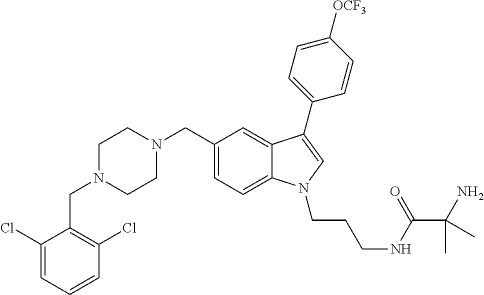 [SMILES: CC(C)(N)C(=O)NCCCn1cc(-c2ccc(OC(F)(F)F)cc2)c2cc(CN3CCN(Cc4c(Cl)cccc4Cl)CC3)ccc21] | 2-amino-N-(3-(5-((4- (2,6- dichlorobenzyl)piperazin- 1-yl)methyl)-3-(4- (trifluoromethoxy)phenyl)- 1H-indol-1-yl)propyl)- 2-methylpropanamide |
| C47 | 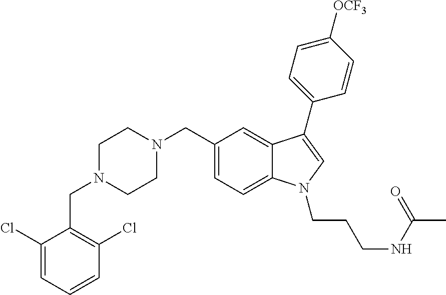 [SMILES: CC(=O)NCCCn1cc(-c2ccc(OC(F)(F)F)cc2)c2cc(CN3CCN(Cc4c(Cl)cccc4Cl)CC3)ccc21] | N-(3-(5-((4-(2,6- dichlorobenzyl)piperazin- 1-yl)methyl)-3-(4- (trifluoromethoxy)phenyl)- 1H-indol-1- yl)propyl)acetamide |
| C50 | 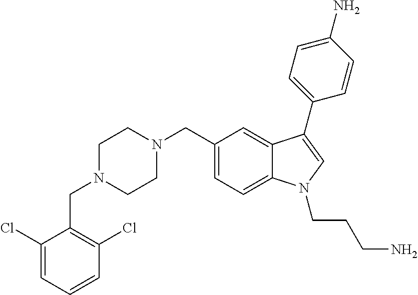 [SMILES: NCCCn1cc(-c2ccc(N)cc2)c2cc(CN3CCN(Cc4c(Cl)cccc4Cl)CC3)ccc21] | 4-(1-(3-aminopropyl)-5- ((4-(2,6- dichlorobenzyl)piperazin- 1-yl)methyl)-1H-indol- 3-yl)amine |
| C51 | 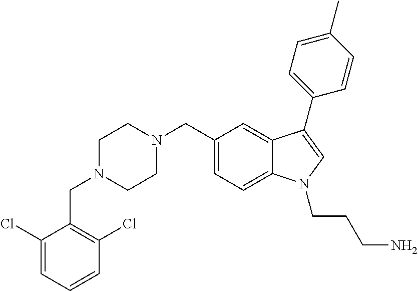 [SMILES: Cc1ccc(-c2cn(CCCN)c3ccc(CN4CCN(Cc5c(Cl)cccc5Cl)CC4)cc23)cc1] | 3-(5-((4-(2,6- dichlorobenzyl)piperazin- 1-yl)methyl)-3-p-tolyl- 1H-indol-1-yl)propan-1- amine |
| C52 | 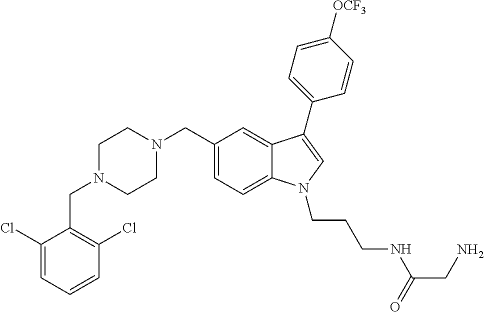 [SMILES: NCC(=O)NCCCn1cc(-c2ccc(OC(F)(F)F)cc2)c2cc(CN3CCN(Cc4c(Cl)cccc4Cl)CC3)ccc21] | 2-amino-N-(3-(5-((4- (2,6- dichlorobenzyl)piperazin- 1-yl)methyl)-3-(4- (trifluoromethoxy)phenyl)- 1H-indol-1- yl)propyl)acetamide |
| C53 | 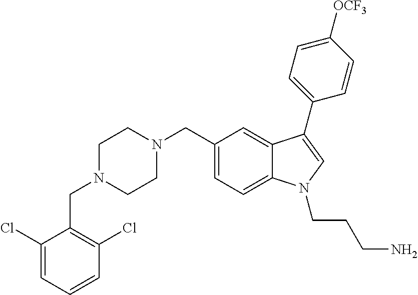 [SMILES: NCCCn1cc(-c2ccc(OC(F)(F)F)cc2)c2cc(CN3CCN(Cc4c(Cl)cccc4Cl)CC3)ccc21] | 2-(5-((4-(2,6- dichlorobenzyl)piperazain- 1-yl)methyl)-3-(4- (trifluoromethoxy)phenyl)- 1H-indol-1- yl)ethanamine |
| C81 | 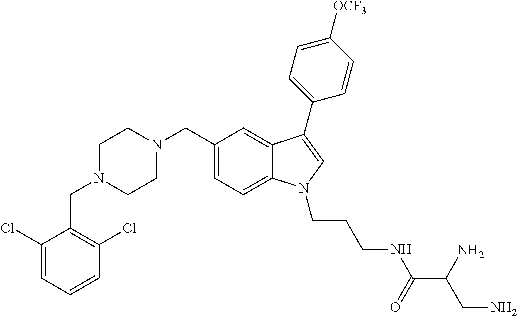 [SMILES: NCC(N)C(=O)NCCCn1cc(-c2ccc(OC(F)(F)F)cc2)c2cc(CN3CCN(Cc4c(Cl)cccc4Cl)CC3)ccc21] | 2,3-diamino-N-(3-(5-((4- (2,6- dichlorobenzyl)piperazin- 1-yl)methyl)-3-(4- (trifluoromethoxy)phenyl)- 1H-indol-1- yl)propyl)propanamide |
| C87 | 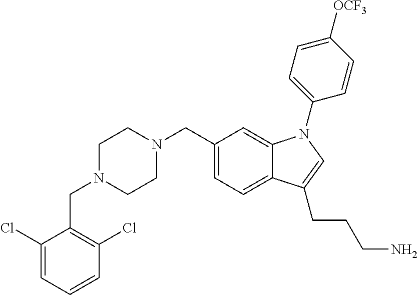 [SMILES: NCCCc1cn(-c2ccc(OC(F)(F)F)cc2)c2cc(CN3CCN(Cc4c(Cl)cccc4Cl)CC3)ccc12] | 3-(6-((4-(2,6- dichlorobenzyl)piperazin- 1-yl)methyl)-1-(4- (trifluoromethoxy)phenyl)- 1H-indol-3-yl)propan- 1-amine |
| C88 | 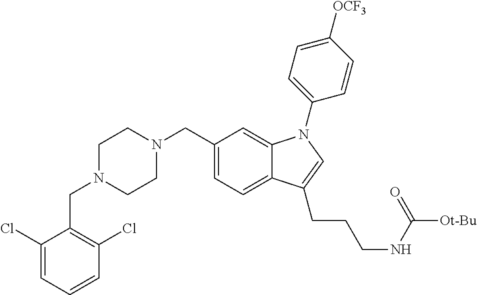 [SMILES: CC(C)(C)OC(=O)NCCCc1cn(-c2ccc(OC(F)(F)F)cc2)c2cc(CN3CCN(Cc4c(Cl)cccc4Cl)CC3)ccc12] | tert-butyl 3-(6-((4-(2,6- dichlorobenzyl)piperazin- 1-yl)methyl)-1-(4- (trifluoromethoxy)phenyl)- 1H-indol-3- yl)propylcarbamate |
| C128 | 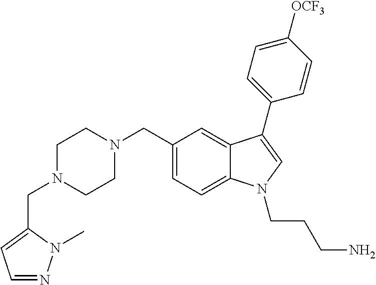 [SMILES: Cn1nccc1CN1CCN(Cc2ccc3c(c2)c(-c2ccc(OC(F)(F)F)cc2)cn3CCCN)CC1] | 3-(5-((4-((1-methyl-1H- pyrazol-5- yl)methyl)piperazin-1- yl)methyl)-3-(4- (trifluoromethoxy)phenyl)- 1H-indol-1-yl)propan- 1-amine |
| C131 | 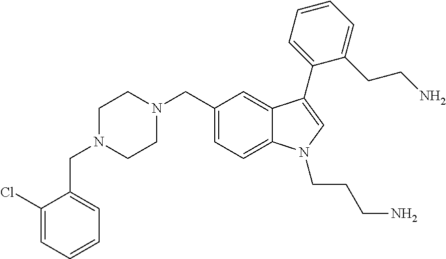 [SMILES: NCCCn1cc(-c2ccccc2CCN)c2cc(CN3CCN(Cc4ccccc4Cl)CC3)ccc21] | 3-(3-(2-(2- aminoethyl)phenyl)-5- ((4-(2- chlorobenzyl)piperazin- 1-yl)methyl)-1H-indol-1- yl)propan-1-amine |
| C132 | 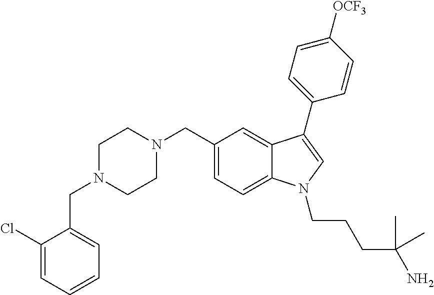 [SMILES: CC(C)(N)CCCn1cc(-c2ccc(OC(F)(F)F)cc2)c2cc(CN3CCN(Cc4ccccc4Cl)CC3)ccc21] | 5-(5-((4-(2- chlorobenzyl)piperazin- 1-yl)methyl)-3-(4- (trifluoromethoxy)phenyl)- 1H-indol-1-yl)-2- methylpentan-2-amine |
| C135 | 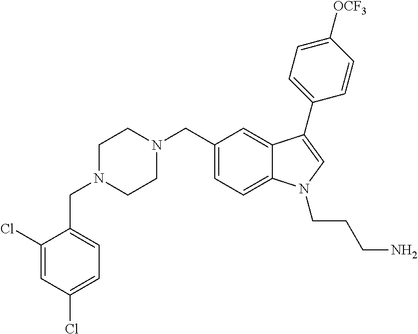 [SMILES: NCCCn1cc(-c2ccc(OC(F)(F)F)cc2)c2cc(CN3CCN(Cc4ccc(Cl)cc4Cl)CC3)ccc21] | 3-(5-((4-(2,4- dichlorobenzy6l)piperazin- 1-yl)methyl)-3-(4- (trifluoromethoxy)phenyl)- 1H-indol-1-yl)propan- 1-amine |
| C136 | 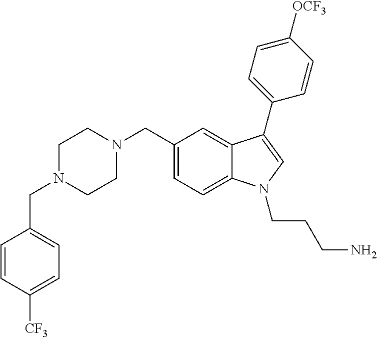 [SMILES: NCCCn1cc(-c2ccc(OC(F)(F)F)cc2)c2cc(CN3CCN(Cc4ccc(C(F)(F)F)cc4)CC3)ccc21] | 3-(3-(4- (trifluoromethoxy)phenyl)- 5-((4-(4- (trifluoromethyl)benzyl) piperazin-1-yl)methyl)- 1H-indol-1-yl)propan-1- amine |
| C137 | 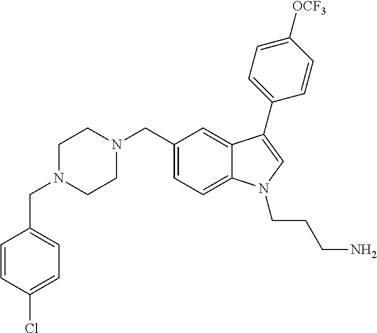 [SMILES: NCCCn1cc(-c2ccc(OC(F)(F)F)cc2)c2cc(CN3CCN(Cc4ccc(Cl)cc4)CC3)ccc21] | 3-(5-((4-(4- chlorobenzyl)piperazin- 1-yl)methyl)-3-(4- (trifluoromethoxy)phenyl)- 1H-indol-1-yl)propan- 1-amine |
| C138 | 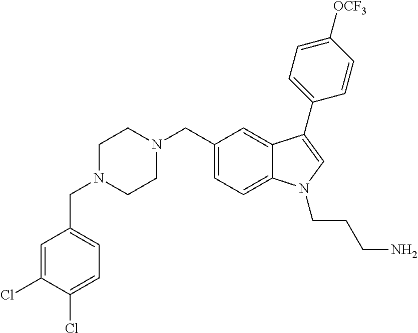 [SMILES: NCCCn1cc(-c2ccc(OC(F)(F)F)cc2)c2cc(CN3CCN(Cc4ccc(Cl)c(Cl)c4)CC3)ccc21] | 3-(5-((4-(3,4- dichlorobenzyl)piperazin- 1-yl)methyl)-3-(4- (trifluoromethoxy)phenyl)- 1H-indol-1-yl)propan- 1-amine |
| C139 | 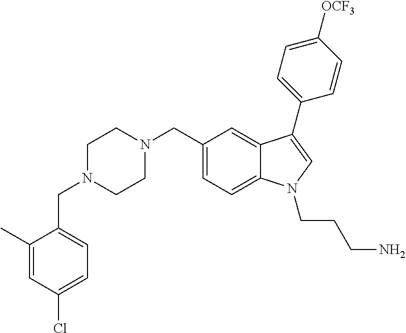 [SMILES: Cc1cc(Cl)ccc1CN1CCN(Cc2ccc3c(c2)c(-c2ccc(OC(F)(F)F)cc2)cn3CCCN)CC1] | 3-(5-((4-(4-chloro-2- methylbenzyl)piperazin- 1-yl)methyl)-3-(4- (trifluoromethoxy)phenyl)- 1H-indol-1-yl)propan- 1-amine |
| C140 | 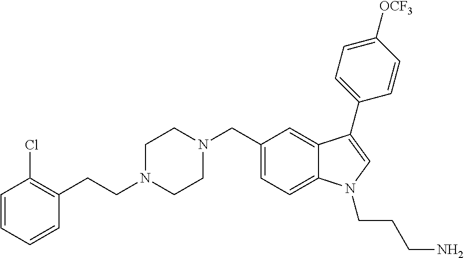 [SMILES: NCCCn1cc(-c2ccc(OC(F)(F)F)cc2)c2cc(CN3CCN(CCc4ccccc4Cl)CC3)ccc21] | 3-(5-((4-(2- chlorophenethyl)piperazin- 1-yl)methyl)-3-(4- (trifluoromethoxy)phenyl)- 1H-indol-1-yl)propan- 1-amine |
| C141 | 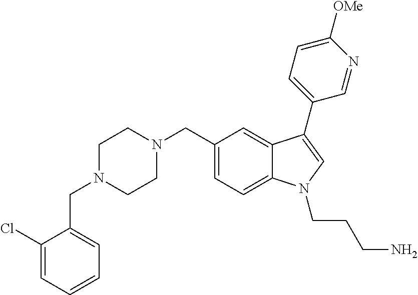 [SMILES: COc1ccc(-c2cn(CCCN)c3ccc(CN4CCN(Cc5ccccc5Cl)CC4)cc23)cn1] | 3-(5-((4-(2- chlorobenzyl)piperazin- 1-yl)methyl)-3-(6- methoxypyridin-3-yl)- 1H-indol-1-yl)propan-1- amine |
| C142 | 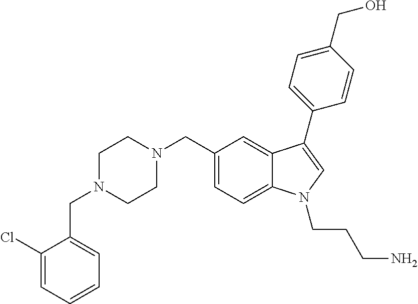 [SMILES: NCCCn1cc(-c2ccc(CO)cc2)c2cc(CN3CCN(Cc4ccccc4Cl)CC3)ccc21] | (4-(1-(3-aminopropyl)-5- ((4-(2- chlorobenzyl)piperazin- 1-yl)methyl)-1H-indol-3- yl)phenyl)methanol |
| C143 | 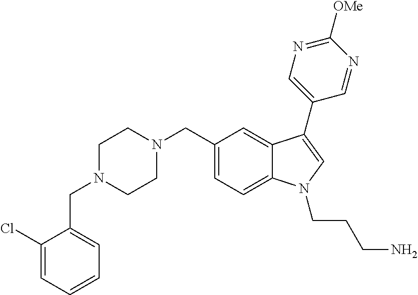 [SMILES: COc1ncc(-c2cn(CCCN)c3ccc(CN4CCN(Cc5ccccc5Cl)CC4)cc23)cn1] | 3-(5-((4-(2- chlorobenzyl)piperazin- 1-yl)methyl)-3-(2- methoxypyrimidin-5-yl)- 1H-indol-1-yl)propan-1- amine |
| C144 | 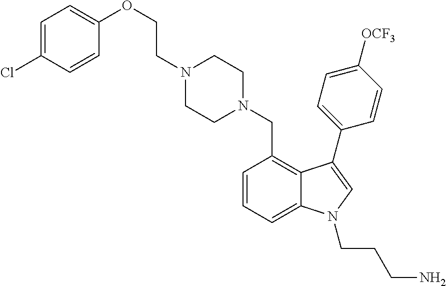 [SMILES: NCCCn1cc(-c2ccc(OC(F)(F)F)cc2)c2c(CN3CCN(CCOc4ccc(Cl)cc4)CC3)cccc21] | 3-(4-((4-(2-(4- chlorophenoxy)ethyl) piperazin-1-yl)methyl)-3-(4- (trifluoromethoxy)phenyl)- 1H-indol-1-yl)propan- 1-amine |
| C145 | 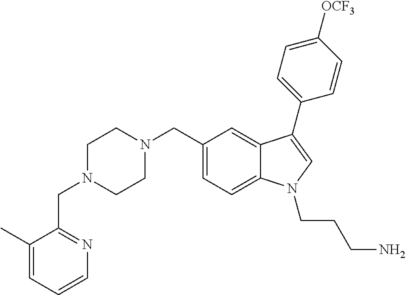 [SMILES: Cc1cccnc1CN1CCN(Cc2ccc3c(c2)c(-c2ccc(OC(F)(F)F)cc2)cn3CCCN)CC1] | 3-(5-((4-((3- methylpyridin-2- yl)methyl)piperazin-1- yl)methyl)-3-(4- (trifluoromethoxy)phenyl)- 1H-indol-1-yl)propan- 1-amine |
| C146 | 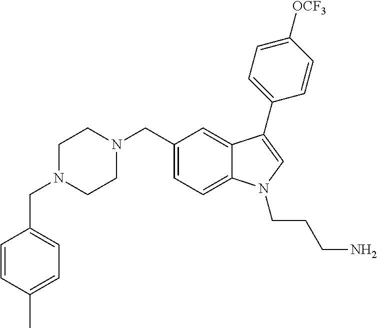 [SMILES: Cc1ccc(CN2CCN(Cc3ccc4c(c3)c(-c3ccc(OC(F)(F)F)cc3)cn4CCCN)CC2)cc1] | 3-(5-((4-(4- methylbenzyl)piperazin- 1-yl)methyl)-3-(4- (trifluoromethoxy)phenyl)- 1H-indol-1-yl)propan- 1-amine |
| C147 | 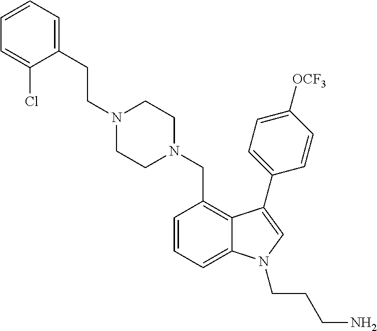 [SMILES: NCCCn1cc(-c2ccc(OC(F)(F)F)cc2)c2c(CN3CCN(CCc4ccccc4Cl)CC3)cccc21] | 3-(4-((4-(2- chlorophenethyl)piperazin- 1-yl)methyl)-3-(4- (trifluoromethoxy)phenyl)- 1H-indol-1-yl)propan- 1-amine |
| C148 | 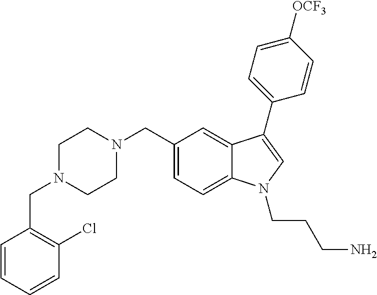 [SMILES: NCCCn1cc(-c2ccc(OC(F)(F)F)cc2)c2cc(CN3CCN(Cc4ccccc4Cl)CC3)ccc21] | (1-(3-aminopropyl)-3-(4- (trifluoromethoxy)phenyl)- 1H-indol-5-yl)(4-(2- chlorobenzyl)piperazin- 1-yl)methanone |
| C151 | 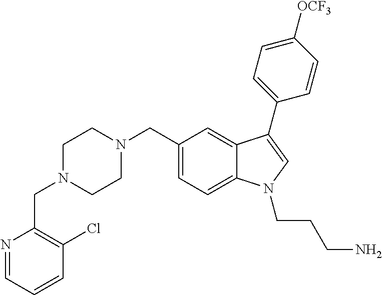 [SMILES: NCCCn1cc(-c2ccc(OC(F)(F)F)cc2)c2cc(CN3CCN(Cc4ncccc4Cl)CC3)ccc21] | 3-(5-((4-((3- chloropyridin-2- yl)methyl)piperazin-1- yl)methyl)-3-(4- (trifluoromethoxy)phenyl)- 1H-indol-1-yl)propan- 1-amine |
| C152 | 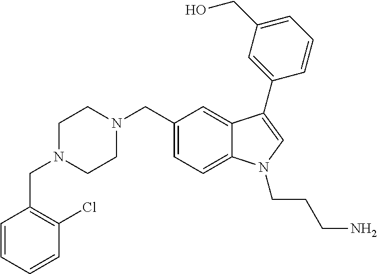 [SMILES: NCCCn1cc(-c2cccc(CO)c2)c2cc(CN3CCN(Cc4ccccc4Cl)CC3)ccc21] | (3-(1-(3-aminopropyl)-5- ((4-(2- chlorobenzyl)piperazin- 1-yl)methyl)-1H-indol-3- yl)phenyl)methanol |
| C153 | 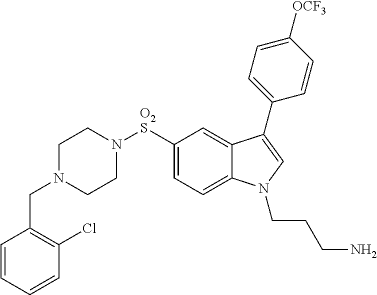 [SMILES: NCCCn1cc(-c2ccc(OC(F)(F)F)cc2)c2cc(S(=O)(=O)N3CCN(Cc4ccccc4Cl)CC3)ccc21] | 3-(5-(4-(2- chlorobenzyl)piperazin- 1-ylsulfonyl)-3-(4- (trifluoromethoxy)phenyl)- 1H-indol-1-yl)propan- 1-amine |
| C154 | 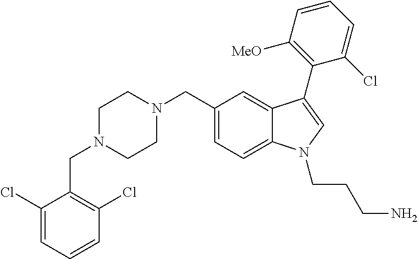 [SMILES: COc1cccc(Cl)c1-c1cn(CCCN)c2ccc(CN3CCN(Cc4c(Cl)cccc4Cl)CC3)cc12] | 3-(3-(2-chloro-6- methoxyphenyl)-5-((4- (2,6- dichlorobenzyl)piperazin- 1-yl)methyl)-1H-indol- 1-yl)propan-1-amine |
| C155 | 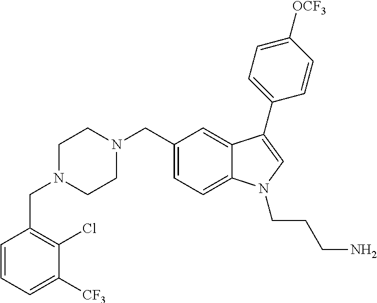 [SMILES: NCCCn1cc(-c2ccc(OC(F)(F)F)cc2)c2cc(CN3CCN(Cc4cccc(C(F)(F)F)c4Cl)CC3)ccc21] | 3-(5-((4-(2-chloro-3- (trifluoromethyl)benzyl) piperazin-1-yl)methyl)-3- (4- (trifluoromethoxy)phenyl)- 1H-indol-1-yl)propan- 1-amine |
| C156 | 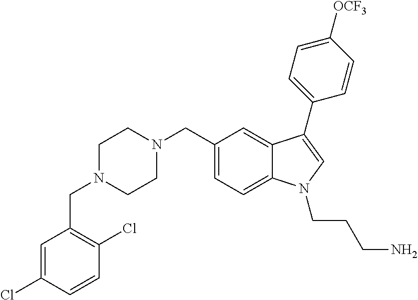 [SMILES: NCCCn1cc(-c2ccc(OC(F)(F)F)cc2)c2cc(CN3CCN(Cc4cc(Cl)ccc4Cl)CC3)ccc21] | 3-(5-((4-(2,5- dichlorobenzyl)piperazin- 1-yl)methyl)-3-(4- (trifluoromethoxy)phenyl)- 1H-indol-1-yl)propan- 1-amine |
| C160 | 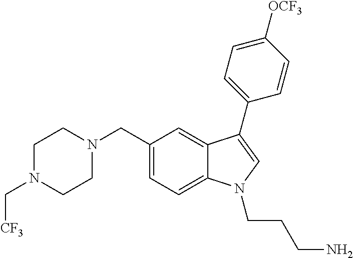 [SMILES: NCCCn1cc(-c2ccc(OC(F)(F)F)cc2)c2cc(CN3CCN(CC(F)(F)F)CC3)ccc21] | 3-(5-((4-(2,2,2- trifluoroethyl)piperazin- 1-yl)methyl)-3-(4- (trifluoromethoxy)phenyl)- 1H-indol-1-yl)propan- 1-amine |
| C161 | 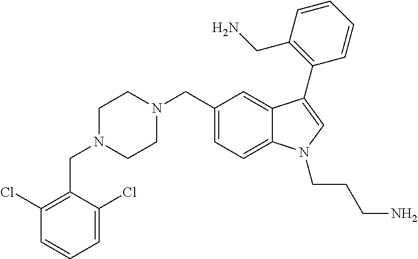 [SMILES: NCCCn1cc(-c2ccccc2CN)c2cc(CN3CCN(Cc4c(Cl)cccc4Cl)CC3)ccc21] | 3-(3-(2- (aminomethyl)phenyl)-5- ((4-(2,6- dichlorobenzyl)piperazin- 1-yl)methyl)-1H-indol- 1-yl)propan-1-amine |
| C162 | 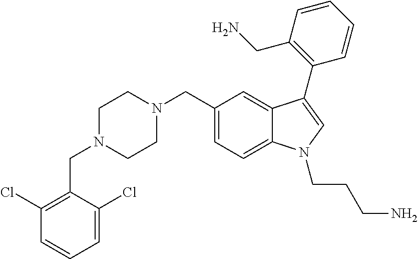 [SMILES: NCCCn1cc(-c2ccccc2CN)c2cc(CN3CCN(Cc4c(Cl)cccc4Cl)CC3)ccc21] | 3-(3-(2- (aminomethyl)phenyl)-5- ((4-(2,6- dichlorobenzyl)piperazin- 1-yl)methyl)-1H-indol- 1-yl)propan-1-amine |
| C163 | 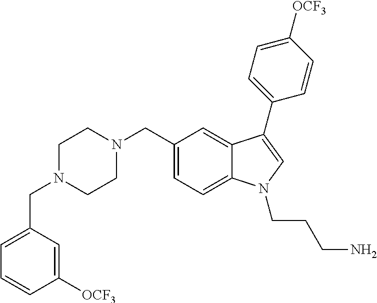 [SMILES: NCCCn1cc(-c2ccc(OC(F)(F)F)cc2)c2cc(CN3CCN(Cc4cccc(OC(F)(F)F)c4)CC3)ccc21] | 3-(5-((4-(3- (trifluoromethoxybenzyl) piperazin-1-yl)methyl)- 3-(4- (trifluoromethoxy)phenyl)- 1H-indol-1-yl)propan- 1-amine |
| C164 | 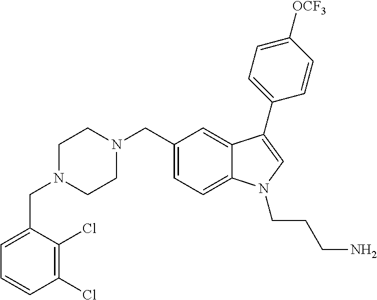 [SMILES: NCCCn1cc(-c2ccc(OC(F)(F)F)cc2)c2cc(CN3CCN(Cc4cccc(Cl)c4Cl)CC3)ccc21] | 3-(5-((4-(2,3- dichlorobenzyl)piperazin- 1-yl)methyl)-3-(4- (trifluoromethoxy)phenyl)- 1H-indol-1-yl)propan- 1-amine |
| C165 | 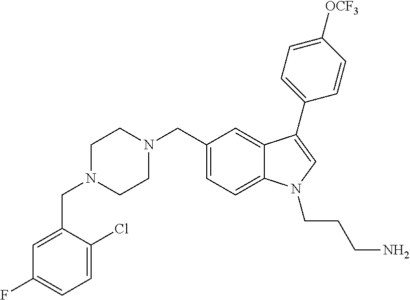 [SMILES: NCCCn1cc(-c2ccc(OC(F)(F)F)cc2)c2cc(CN3CCN(Cc4cc(F)ccc4Cl)CC3)ccc21] | 3-(5-((4-(2-chloro-5- fluorobenzyl)piperazin- 1-yl)methyl)-3-(4- (trifluoromethoxy)phenyl)- 1H-indol-1-yl)propan- 1-amine |
| C166 | 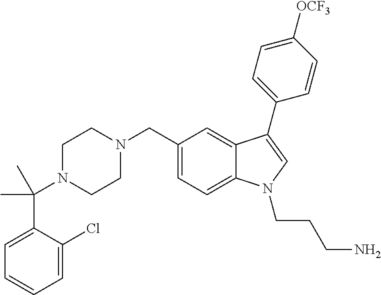 [SMILES: CC(C)(c1ccccc1Cl)N1CCN(Cc2ccc3c(c2)c(-c2ccc(OC(F)(F)F)cc2)cn3CCCN)CC1] | 3-(5-((4-(2-(2- chlorophenyl)propan-2- yl)piperazin-1- yl)methyl)-3-(4- (trifluoromethoxy)phenyl)- 1H-indol-1-yl)propan- 1-amine |
| C167 | 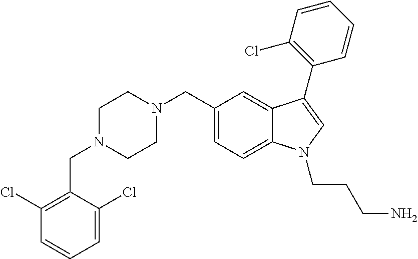 [SMILES: NCCCn1cc(-c2ccccc2Cl)c2cc(CN3CCN(Cc4c(Cl)cccc4Cl)CC3)ccc21] | 3-(3-(2-chlorophenyl)-5- ((4-(2,6- dichlorobenzyl)piperazin- 1-yl)methyl)-1H-indol- 1-yl)propan-1-amine |
| C168 | 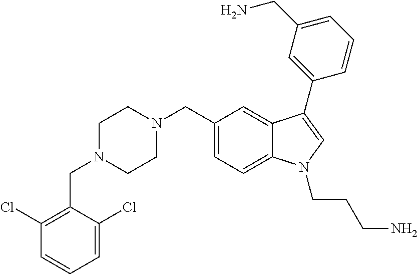 [SMILES: NCCCn1cc(-c2cccc(CN)c2)c2cc(CN3CCN(Cc4c(Cl)cccc4Cl)CC3)ccc21] | 3-(3-(3- (aminomethyl)phenyl)-5- ((4-(2,6- dichlorobenzyl)piperazin- 1-yl)methyl)-1H-indol- 1-yl)propan-1-amine |

In some embodiments, the compound of Formula (VIIa) is selected from any one of the compounds from the following table:

TABLE 11B

| Compound | | |
|---|---|---|
| Ref. | Compound | Chemical Name |
| C179 | 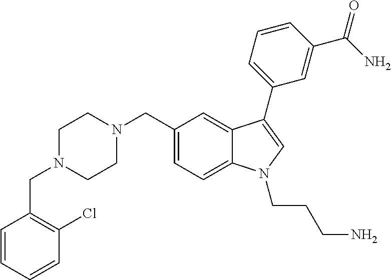 [SMILES: NCCCn1cc(-c2cccc(C(N)=O)c2)c2cc(CN3CCN(Cc4ccccc4Cl)CC3)ccc21] | 3-(1-(3-aminopropyl)-5- ((4-(2- chlorobenzyl)piperazin-1- yl)methyl)-1H-indol-3- yl)benzamide |
| C180 | 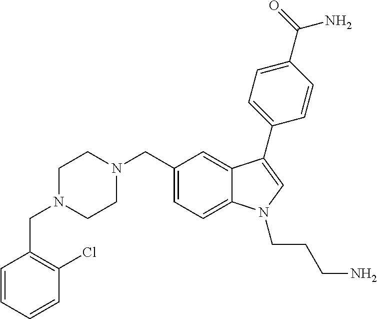 [SMILES: NCCCn1cc(-c2ccc(C(N)=O)cc2)c2cc(CN3CCN(Cc4ccccc4Cl)CC3)ccc21] | 4-(1-(3-aminopropyl)-5- ((4-(2- chlorobenzyl)piperazin-1- yl)methyl)-1H-indol-3- yl)benzamide |
| C181 | 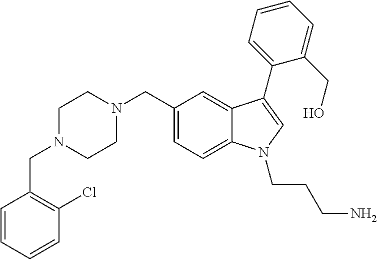 [SMILES: NCCCn1cc(-c2ccccc2CO)c2cc(CN3CCN(Cc4ccccc4Cl)CC3)ccc21] | (2-(1-(3-aminopropyl)-5- ((4-(2- chlorobenzyl)piperazin-1- yl)methyl)-1H-indol-3- yl)phenyl)methanol |
| C183 | 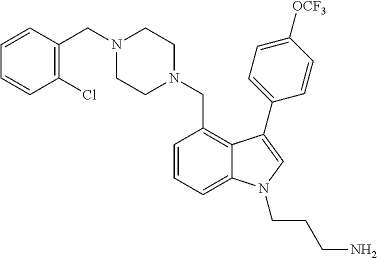 [SMILES: NCCCn1cc(-c2ccc(OC(F)(F)F)cc2)c2c(CN3CCN(Cc4ccccc4Cl)CC3)cccc21] | 3-(4-((4-(2- chlorobenzyl)piperazin-1- yl)methyl)-3-(4- (trifluoromethoxy)phenyl)- 1H-indol-1-yl)propan-1- amine |
| C184 | 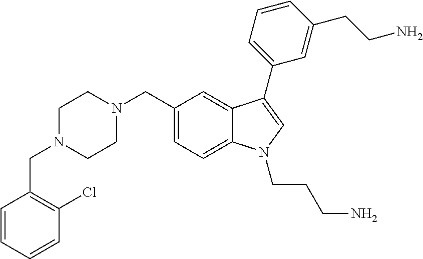 [SMILES: NCCCn1cc(-c2cccc(CCN)c2)c2cc(CN3CCN(Cc4ccccc4Cl)CC3)ccc21] | 3-(3-(3-(2- aminoethyl)phenyl)-4-((4- (2-chlorobenzyl)piperazin- 1-yl)methyl)-1H-indol-1- yl)propan-1-amine |
| C185 | 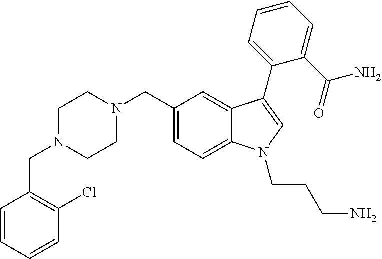 [SMILES: NCCCn1cc(-c2ccccc2C(N)=O)c2cc(CN3CCN(Cc4ccccc4Cl)CC3)ccc21] | 2-(1-(3-aminopropyl)-5- ((4-(2- chlorobenzyl)piperazin-1- yl)methyl)-1H-indol-3- yl)benzamide |
| C186 | 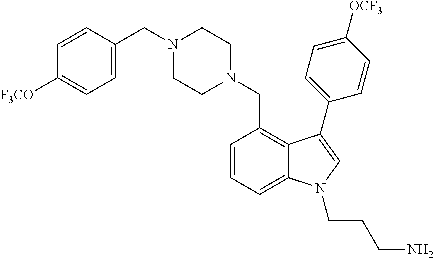 [SMILES: NCCCn1cc(-c2ccc(OC(F)(F)F)cc2)c2c(CN3CCN(Cc4ccc(OC(F)(F)F)cc4)CC3)cccc21] | 3-(4-((4-(4- (trifluoromethoxy)benzyl) piperazin-1-yl)methyl)-3-(4- (trifluoromethoxy)phenyl)- 1H-indol-1-yl)propan-1- amine |
| C187 | 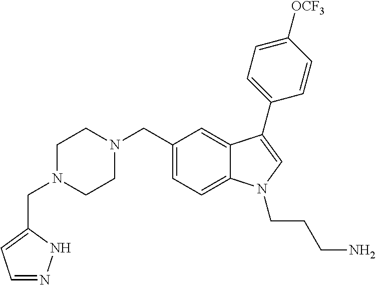 [SMILES: NCCCn1cc(-c2ccc(OC(F)(F)F)cc2)c2cc(CN3CCN(Cc4ccn[nH]4)CC3)ccc21] | 3-(5-((4-((1H-pyrazol-5- yl)methyl)piperazin-1- yl)methyl)-3-(4- (trifluoromethoxy)phenyl)- 1H-indol-1-yl)propan-1- amine |
| C188 | 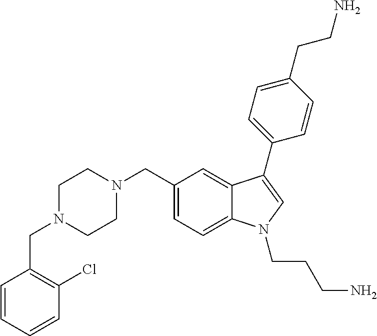 [SMILES: NCCCn1cc(-c2ccc(CCN)cc2)c2cc(CN3CCN(Cc4ccccc4Cl)CC3)ccc21] | 3-(3-(4-(2- aminoethyl)phenyl)-5-((4- (2-chlorobenzyl)piperazin- 1-yl)methyl)-1H-indol-1- yl)propan-1-amine |
| C189 | 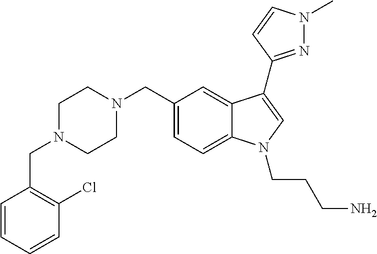 [SMILES: Cn1ccc(-c2cn(CCCN)c3ccc(CN4CCN(Cc5ccccc5Cl)CC4)cc23)n1] | 3-(5-((4-(2- chlorobenzyl)piperazin-1- yl)methyl)-3-(1-methyl- 1H-pyrazol-3-yl)-1H- indol-1-yl)propan-1-amine |
| C190 | 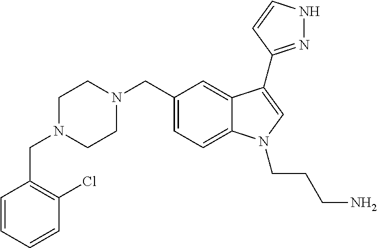 [SMILES: NCCCn1cc(-c2cc[nH]n2)c2cc(CN3CCN(Cc4ccccc4Cl)CC3)ccc21] | 3-(5-((4-(2- chlorobenzyl)piperazin-1- yl)methyl)-3-(1H-pyrazol- 3-yl)-1H-indol-1- yl)propan-1-amine |
| C191 | 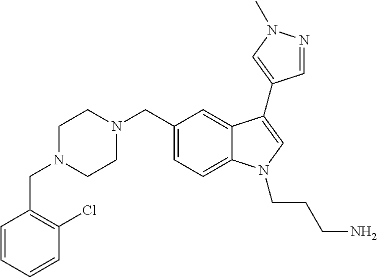 [SMILES: Cn1cc(-c2cn(CCCN)c3ccc(CN4CCN(Cc5ccccc5Cl)CC4)cc23)cn1] | 3-(5-((4-(2- chlorobenzyl)piperazin-1- yl)methyl)-3-(1-methyl- 1H-pyrazol-4-yl)-1H- indol-1-yl)propan-1-amine |
| C192 | 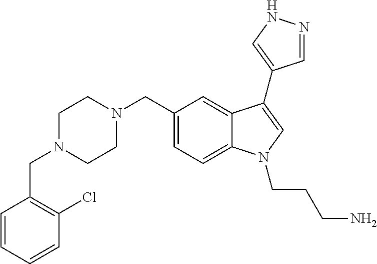 [SMILES: NCCCn1cc(-c2cn[nH]c2)c2cc(CN3CCN(Cc4ccccc4Cl)CC3)ccc21] | 3-(5-((4-(2- chlorobenzyl)piperazin-1- yl)methyl)-3-(1H-pyrazol- 4-yl)-1H-indol-1- yl)propan-1-amine |
| C194 | 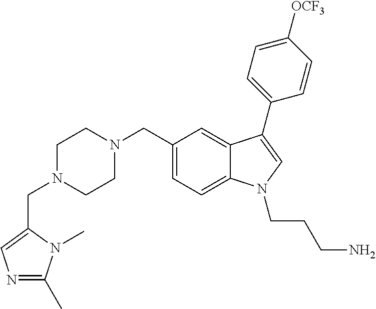 [SMILES: Cc1ncc(CN2CCN(Cc3ccc4c(c3)c(-c3ccc(OC(F)(F)F)cc3)cn4CCCN)CC2)n1C] | 3-(5-((4-((1,2-dimethyl- 1H-imidazol-5- yl)methyl)piperazin-1- yl)methyl)-3-(4- (trifluoromethoxy)phenyl)- 1H-indol-1-yl)propan-1- amine |
| C195 | 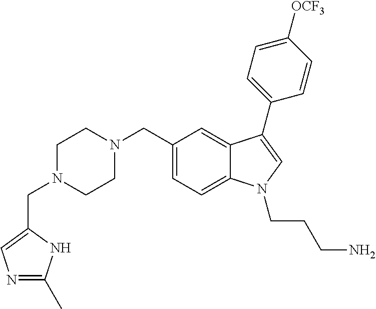 [SMILES: Cc1ncc(CN2CCN(Cc3ccc4c(c3)c(-c3ccc(OC(F)(F)F)cc3)cn4CCCN)CC2)[nH]1] | 3-(5-((4-((2-methyl-1H- imidazol-5- yl)methyl)piperazin-1- yl)methyl)-3-(4- (trifluoromethoxy)phenyl)- 1H-indol-1-yl)propan-1- amine |
| C196 | 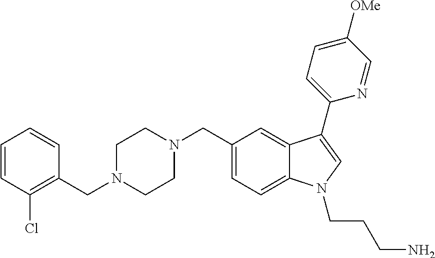 [SMILES: COc1ccc(-c2cn(CCCN)c3ccc(CN4CCN(Cc5ccccc5Cl)CC4)cc23)nc1] | 3-(5-((4-(2- chlorobenzyl)piperazin-1- yl)methyl)-3-(5- methoxypyridin-2-yl)-1H- indol-1-yl)propan-1-amine |
| C199 | 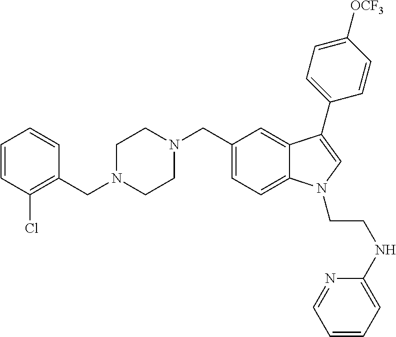 [SMILES: FC(F)(F)Oc1ccc(-c2cn(CCNc3ccccn3)c3ccc(CN4CCN(Cc5ccccc5Cl)CC4)cc23)cc1] | N-(2-(5-((4-(2- chlorobenzyl)piperazin-1- yl)methyl)-3-(4- (trifluoromethoxy)phenyl)- 1H-indol-1- yl)ethyl)pyridin-2-amine |
| C202 | 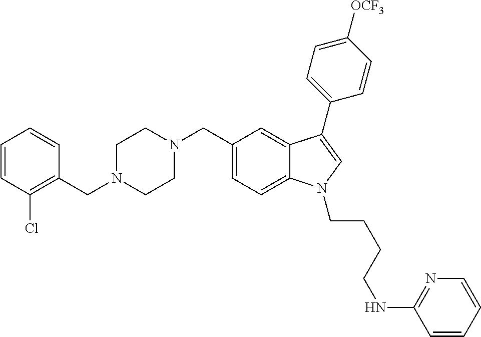 [SMILES: FC(F)(F)Oc1ccc(-c2cn(CCCCNc3ccccn3)c3ccc(CN4CCN(Cc5ccccc5Cl)CC4)cc23)cc1] | N-(4-(5-((4-(2- chlorobenzyl)piperazin-1- yl)methyl)-3-(4- (trifluoromethoxy)phenyl)- 1H-indol-1- yl)butyl)pyridin-2-amine |
| C204 | 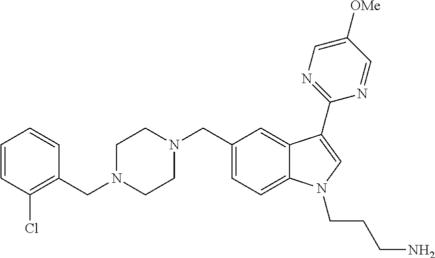 [SMILES: COc1cnc(-c2cn(CCCN)c3ccc(CN4CCN(Cc5ccccc5Cl)CC4)cc23)nc1] | 3-(5-((4-(2- chlorobenzyl)piperazin-1- yl)methyl)-3-(5- methoxypyrimidin-2-yl)- 1H-indol-1-yl)propan-1- amine |
| C205 | 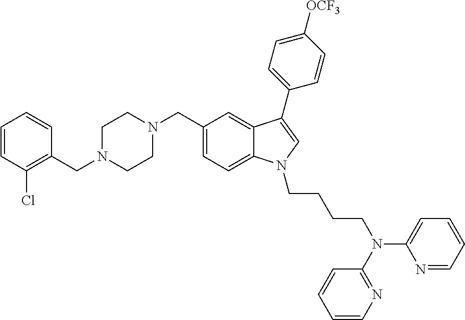 [SMILES: FC(F)(F)Oc1ccc(-c2cn(CCCCN(c3ccccn3)c3ccccn3)c3ccc(CN4CCN(Cc5ccccc5Cl)CC4)cc23)cc1] | N-(4-(5-((4-(2- chlorobenzyl)piperazin-1- yl)methyl)-3-(4- (trifluoromethoxy)phenyl)- 1H-indol-1-yl)butyl)-N- (pyridin-2-yl)pyridin-2- amine |
| C208 | 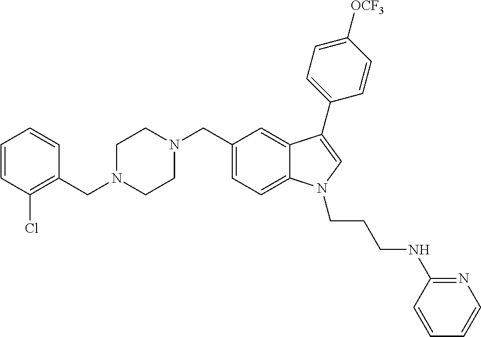 [SMILES: FC(F)(F)Oc1ccc(-c2cn(CCCNc3ccccn3)c3ccc(CN4CCN(Cc5ccccc5Cl)CC4)cc23)cc1] | N-(3-(5-((4-(2- chlorobenzyl)piperazin-1- yl)methyl)-3-(4- (trifluoromethoxy)phenyl)- 1H-indol-1- yl)propyl)pyridin-2-amine |
| C222 | 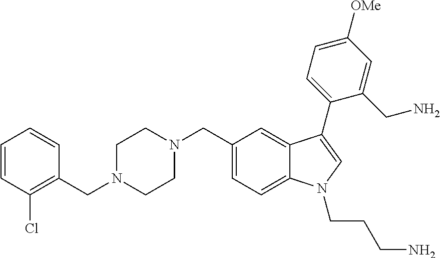 [SMILES: COc1ccc(-c2cn(CCCN)c3ccc(CN4CCN(Cc5ccccc5Cl)CC4)cc23)c(CN)c1] | 3-(3-(2-(aminomethyl)-4- methoxyphenyl)-5-((4-(2- chlorobenzyl)piperazin-1- yl)methyl)-1H-indol-1- yl)propan-1-amine |
| C223 | 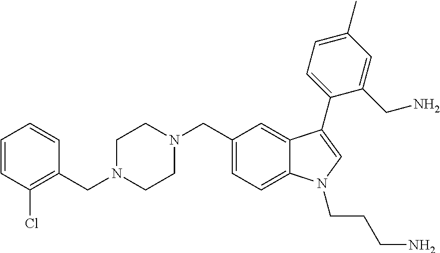 [SMILES: Cc1ccc(-c2cn(CCCN)c3ccc(CN4CCN(Cc5ccccc5Cl)CC4)cc23)c(CN)c1] | 3-(3-(2-(aminomethyl)-4- methylphenyl)-5-((4-(2- chlorobenzyl)piperazin-1- yl)methyl)-1H-indol-1- yl)propan-1-amine |
| C238 | 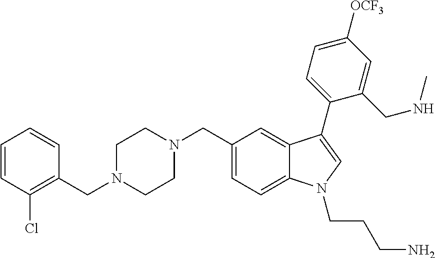 [SMILES: CNCc1cc(OC(F)(F)F)ccc1-c1cn(CCCN)c2ccc(CN3CCN(Cc4ccccc4Cl)CC3)cc12] | 3-(5-((4-(2- chlorobenzyl)piperazin-1- yl)methyl)-3-(2- ((methylamino)methyl)-4- (trifluoromethoxy)phenyl)- 1H-indol-1-yl)propan-1- amine |
| C239 | 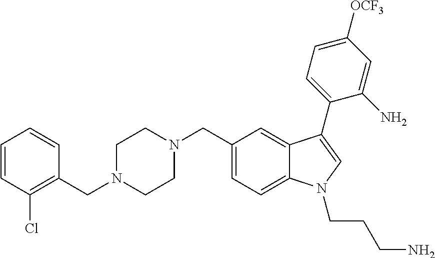 [SMILES: NCCCn1cc(-c2ccc(OC(F)(F)F)cc2N)c2cc(CN3CCN(Cc4ccccc4Cl)CC3)ccc21] | 2-(1-(3-aminopropyl)-5- ((4-(2- chlorobenzyl)piperazin-1- yl)methyl)-1H-indol-3-yl)- 5-(trifluoromethoxy)aniline |
| C240 | 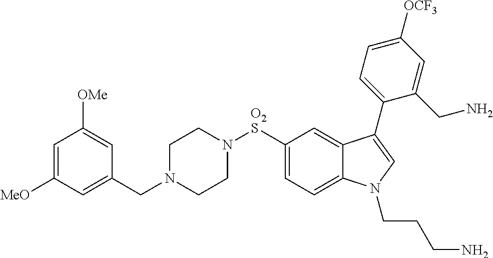 [SMILES: COc1cc(CN2CCN(S(=O)(=O)c3ccc4c(c3)c(-c3ccc(OC(F)(F)F)cc3CN)cn4CCCN)CC2)cc(OC)c1] | 3-(3-(2-(aminomethyl)-4- (trifluoromethoxy)phenyl)- 5-((4-(3,5- dimethoxybenzyl)piperazin- 1-yl)sulfonyl)-1H-indol-1- yl)propan-1-amine |
| C245 | 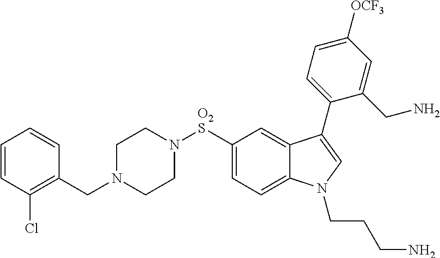 [SMILES: NCCCn1cc(-c2ccc(OC(F)(F)F)cc2CN)c2cc(S(=O)(=O)N3CCN(Cc4ccccc4Cl)CC3)ccc21] | 3-(3-(2-(aminomethyl)-4- (trifluoromethoxy)phenyl)- 5-((4-(2- chlorobenzyl)piperazin-1- yl)sulfonyl)-1H-indol-1- yl)propan-1-amine |
| C251 | 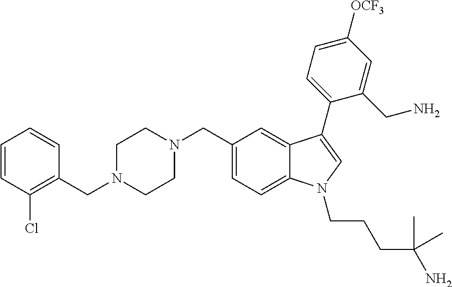 [SMILES: CC(C)(N)CCCn1cc(-c2ccc(OC(F)(F)F)cc2CN)c2cc(CN3CCN(Cc4ccccc4Cl)CC3)ccc21] | 5-(3-(2-(aminomethyl)-4- (trifluoromethoxy)phenyl)- 5-((4-(2- chlorobenzyl)piperazin-1- yl)methyl)-1H-indol-1-yl)- 2-methylpentan-2-amine |
| C266 | 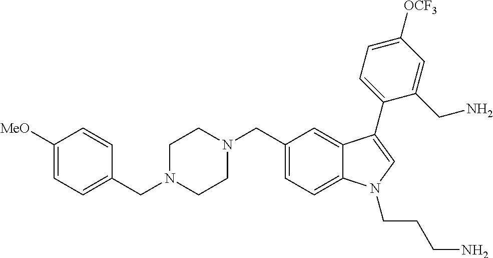 [SMILES: COc1ccc(CN2CCN(Cc3ccc4c(c3)c(-c3ccc(OC(F)(F)F)cc3CN)cn4CCCN)CC2)cc1] | 3-(3-(2-(aminomethyl)-4- (trifluoromethoxy)phenyl)- 5-((4-(4- methoxybenzyl)piperazin- 1-yl)methyl)-1H-indol-1- yl)propan-1-amine |
| C267 | 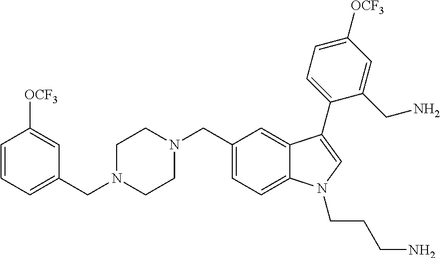 [SMILES: NCCCn1cc(-c2ccc(OC(F)(F)F)cc2CN)c2cc(CN3CCN(Cc4cccc(OC(F)(F)F)c4)CC3)ccc21] | 3-(3-(2-(aminomethyl)-4- (trifluoromethoxy)phenyl)- 5-((4-(3- (trifluoromethoxy)benzyl) piperazin-1-yl)methyl)-1H- indol-1-yl)propan-1-amine |
| C271 | 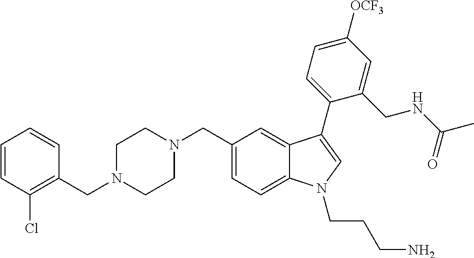 [SMILES: CC(=O)NCc1cc(OC(F)(F)F)ccc1-c1cn(CCCN)c2ccc(CN3CCN(Cc4ccccc4Cl)CC3)cc12] | N-(2-(1-(3-aminopropyl)- 5-((4-(2- chlorobenzyl)piperazin-1- yl)methyl)-1H-indolo-3-yl)- 5- (trifluoromethoxy)benzyl) acetamide |
| C283 | 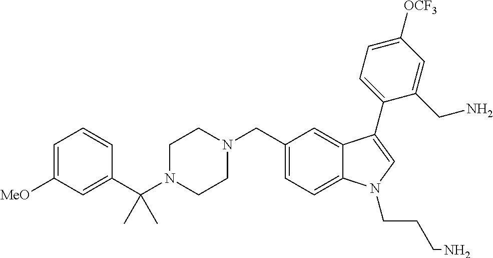 [SMILES: COc1cccc(C(C)(C)N2CCN(Cc3ccc4c(c3)c(-c3ccc(OC(F)(F)F)cc3CN)cn4CCCN)CC2)c1] | 3-(3-(2-(aminomethyl)-4- (trifluoromethoxy)phenyl)- 5-((4-(2-(3- methoxyphenyl)propan-2- yl)piperazin-1-yl)methyl)- 1H-indol-1-yl)propan-1- amine |
| C284 | 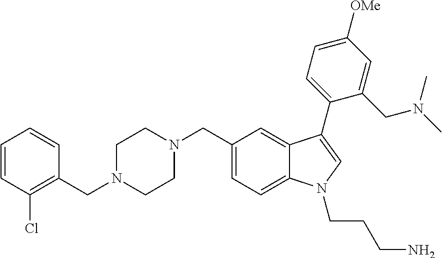 [SMILES: COc1ccc(-c2cn(CCCN)c3ccc(CN4CCN(Cc5ccccc5Cl)CC4)cc23)c(CN(C)C)c1] | 3-(5-((4-(2- chlorobenzyl)piperazin-1- yl)methyl)-3-(2- ((dimethylamino)methyl)- 4-methoxyphenyl)-1H- indol-1-yl)propan-1-amine |
| C285 | 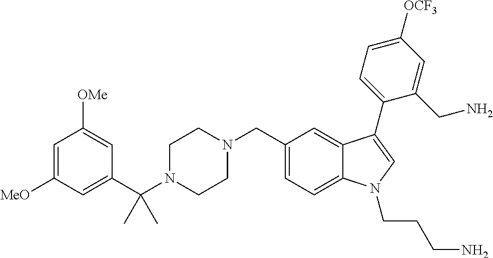 [SMILES: COc1cc(OC)cc(C(C)(C)N2CCN(Cc3ccc4c(c3)c(-c3ccc(OC(F)(F)F)cc3CN)cn4CCCN)CC2)c1] | 3-(3-(2-(aminomethyl)-4- (trifluoromethoxy)phenyl)- 5-((4-(2-(3,5- dimethoxyphenyl)propan- 2-yl)piperazin-1- yl)methyl)-1H-indol-1- yl)propan-1-amine |
| C287 | 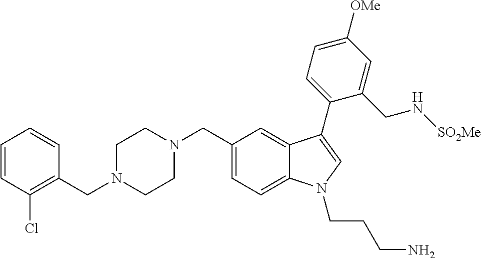 [SMILES: COc1ccc(-c2cn(CCCN)c3ccc(CN4CCN(Cc5ccccc5Cl)CC4)cc23)c(CNS(C)(=O)=O)c1] | N-(2-(1-(3-aminopropyl)- 5-((4-(2- chlorobenzyl)piperazin-1- yl)methyl)-1H-indol-3-yl)- 5- methoxybenzyl) methanesulfonamide |
| C289 | 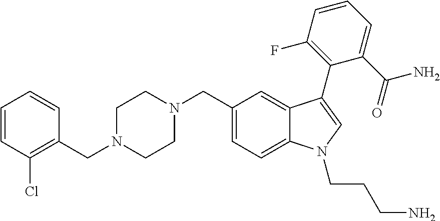 [SMILES: NCCCn1cc(-c2c(F)cccc2C(N)=O)c2cc(CN3CCN(Cc4ccccc4Cl)CC3)ccc21] | 2-(1-(3-aminopropyl)-5- ((4-(2- chlorobenzyl)piperazin-1- yl)methyl)-1H-indol-3-yl)- 3-fluorobenzamide |
| C302 | 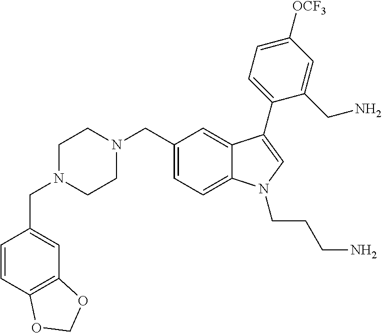 [SMILES: NCCCn1cc(-c2ccc(OC(F)(F)F)cc2CN)c2cc(CN3CCN(Cc4ccc5c(c4)OCO5)CC3)ccc21] | 3-(3-(2-(aminomethyl)-4- (trifluoromethoxy)phenyl)- 5-((4-(benzo[d][1,3]dioxol- 5-ylmethyl)piperazin-1- yl)methyl)-1H-indol-1- yl)propan-1-amine |
| C303 | 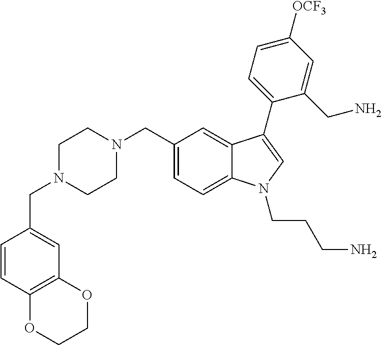 [SMILES: NCCCn1cc(-c2ccc(OC(F)(F)F)cc2CN)c2cc(CN3CCN(Cc4ccc5c(c4)OCCO5)CC3)ccc21] | 3-(3-(2-(aminomethyl)-4- (trifluoromethoxy)phenyl)- 5-((4-((2,3- dihydrobenzo[d][1,4]dioxin- 6-yl)methyl)piperazin-1- yl)methyl)-1H-indol-1- yl)propan-1-amine |
| C304 | 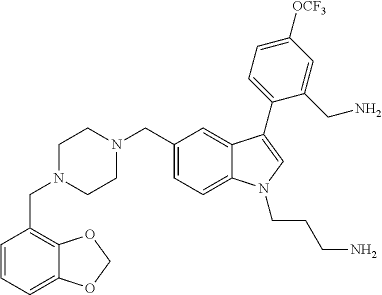 [SMILES: NCCCn1cc(-c2ccc(OC(F)(F)F)cc2CN)c2cc(CN3CCN(Cc4cccc5c4OCO5)CC3)ccc21] | 3-(3-(2-(aminomethyl)-4- (trifluoromethoxy)phenyl)- 5-((4-(benzo[d][1,3]dioxol- 4-ylmethyl)piperazin-1- yl)methyl)-1H-indol-1- yl)propan-1-amine |
| C305 | 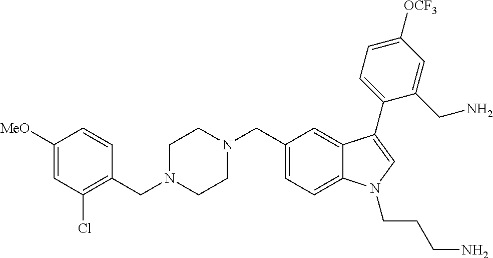 [SMILES: COc1ccc(CN2CCN(Cc3ccc4c(c3)c(-c3ccc(OC(F)(F)F)cc3CN)cn4CCCN)CC2)c(Cl)c1] | 3-(3-(2-(aminomethyl)-4- (trifluoromethoxy)phenyl)- 5-((4-(2-chloro-4- methoxybenzyl)piperazin- 1-yl)methyl)-1H-indol-1- yl)propan-1-amine |
| C306 | 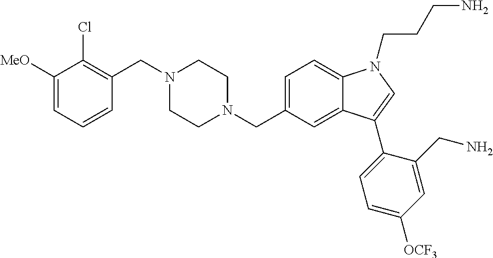 [SMILES: COc1cccc(CN2CCN(Cc3ccc4c(c3)c(-c3ccc(OC(F)(F)F)cc3CN)cn4CCCN)CC2)c1Cl] | 3-(3-(2-(aminomethyl)-4- (trifluoromethoxy)phenyl)- 5-((4-(2-chloro-3- methoxybenzyl)piperazin- 1-yl)methyl)-1H-indol-1- yl)propan-1-amine |
| C307 | 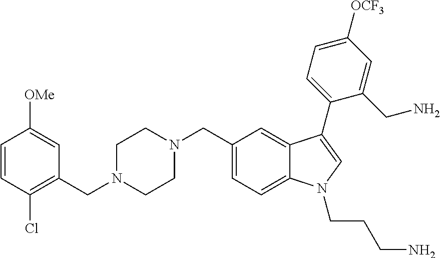 [SMILES: COc1ccc(Cl)c(CN2CCN(Cc3ccc4c(c3)c(-c3ccc(OC(F)(F)F)cc3CN)cn4CCCN)CC2)c1] | 3-(3-(2-(aminomethyl)-4- (trifluoromethoxy)phenyl)- 5-((4-(2-chloro-5- methoxybenzyl)piperazin- 1-yl)methyl)-1H-indol-1- yl)propan-1-amine |

In one aspect, provided herein is a compound of Formula (VIIIa), or a pharmaceutically acceptable salt, or solvate thereof:

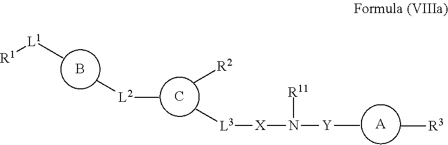

wherein,

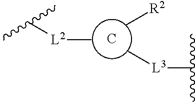

is a bicyclic heteroaryl or heterocycloalkyl that is selected from the following structures:

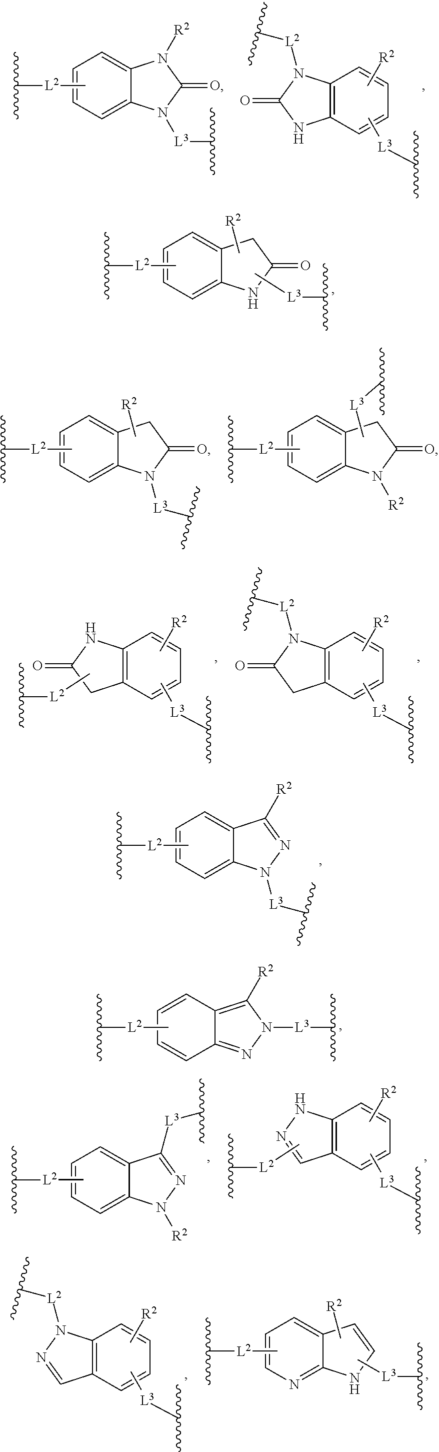

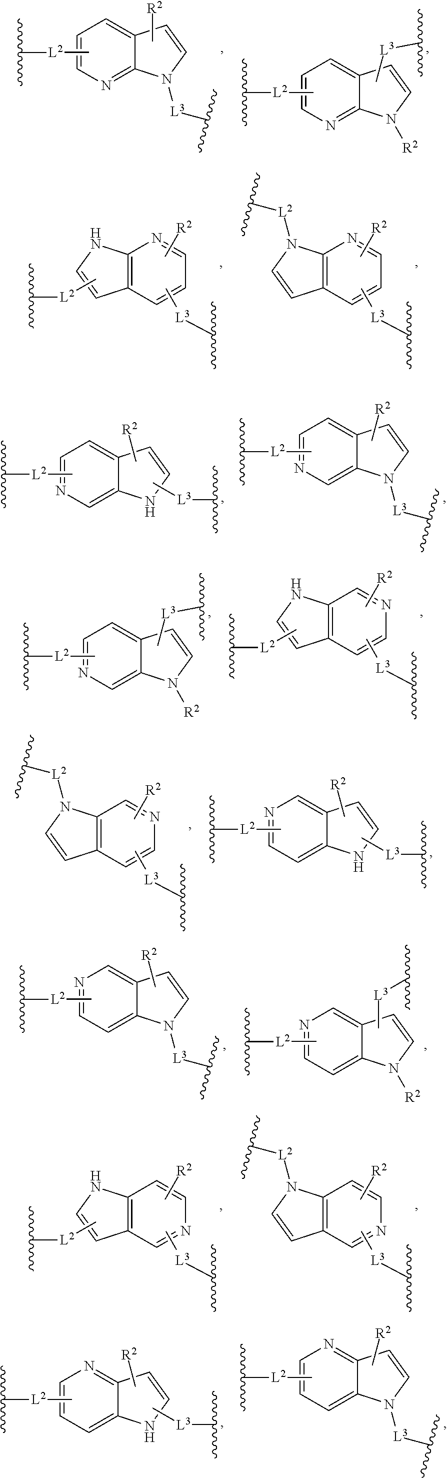

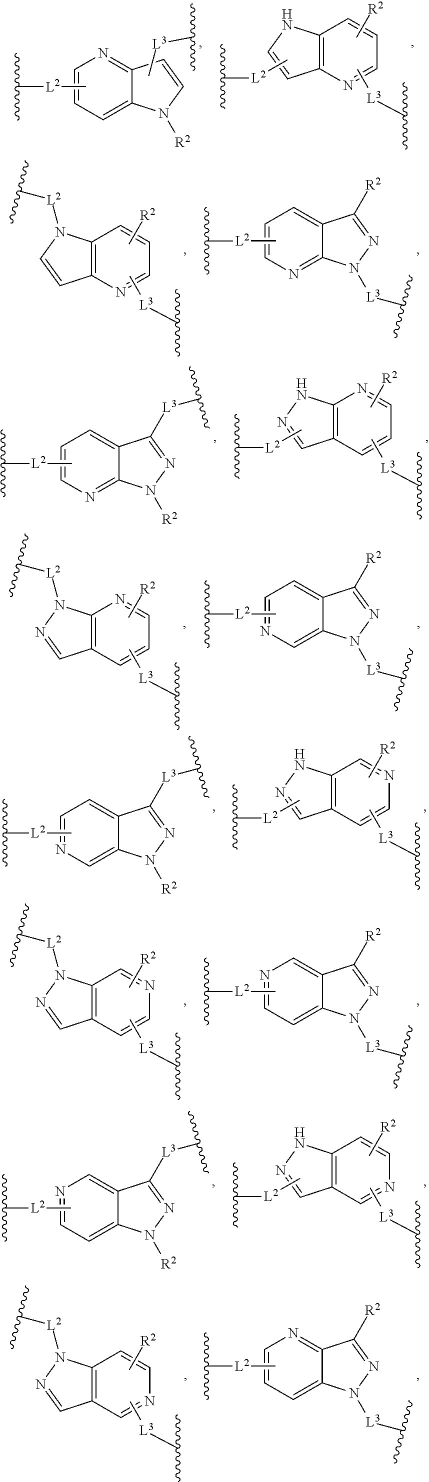

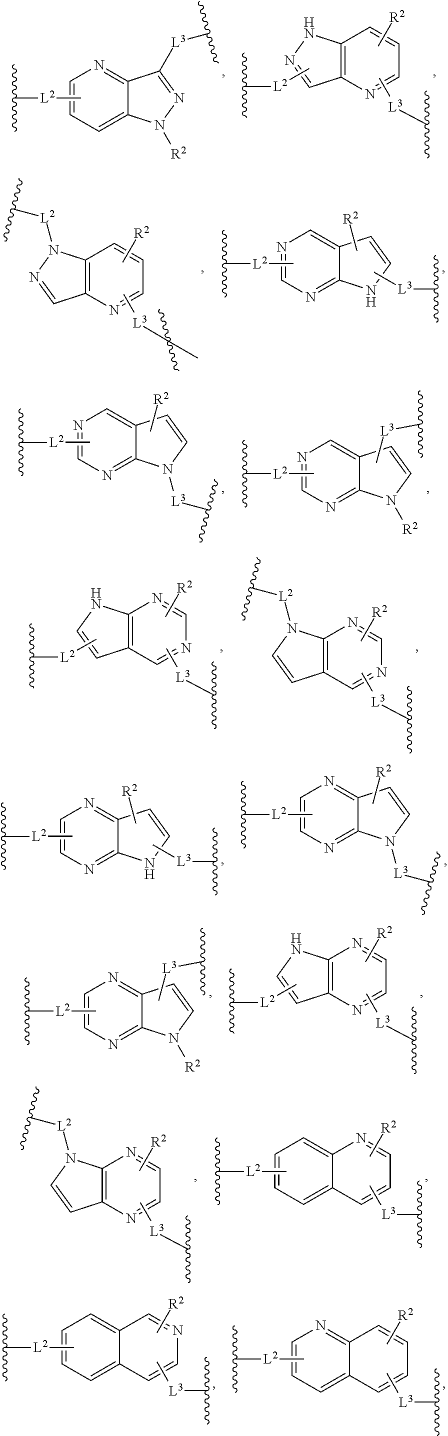

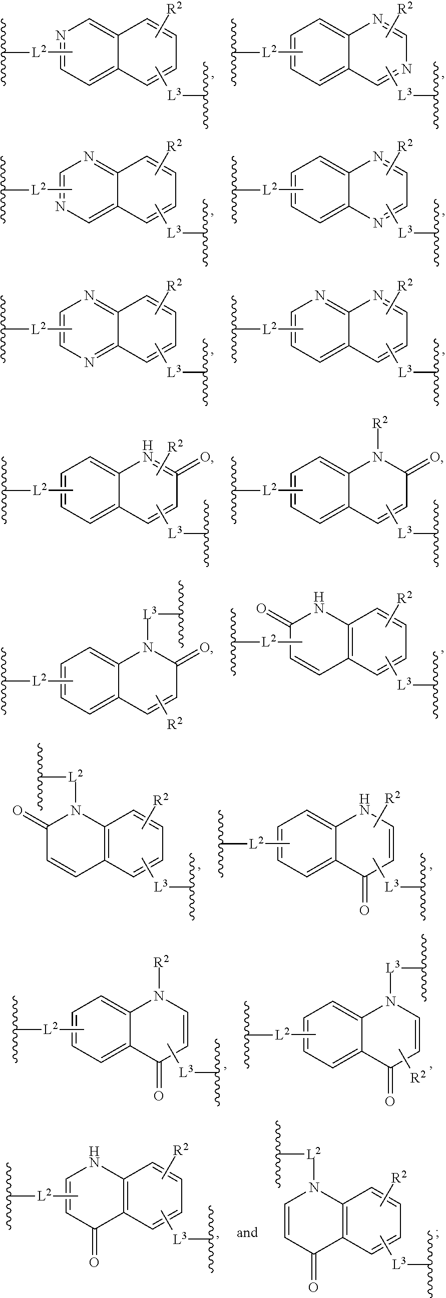

L¹ and L² are each independently an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N;
wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted C₃-C₆cycloalkylene; an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)— or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N; wherein if Ring A is substituted, then Ring A is substituted with at least one R^{A};
each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), and optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring, and
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring,
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl).

In some embodiments,

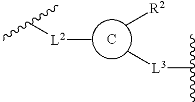

is selected from:

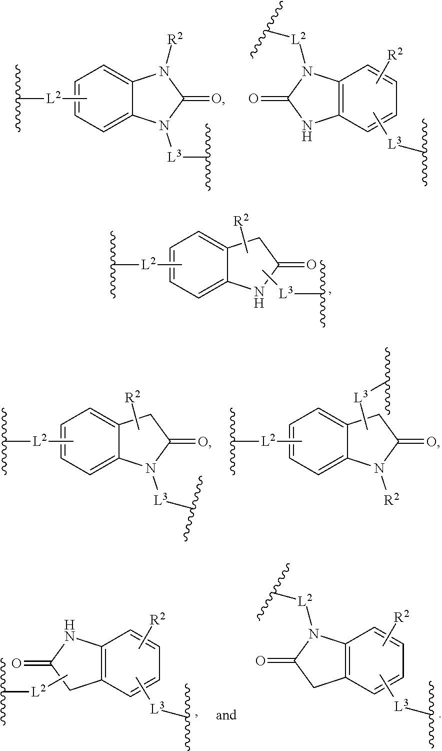

In some embodiments,

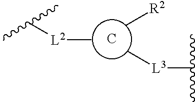

is selected from:

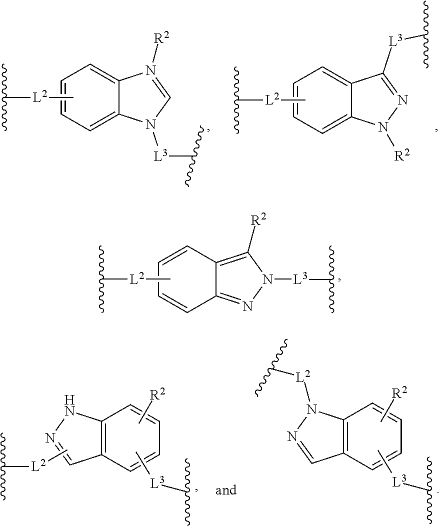

In some embodiments,

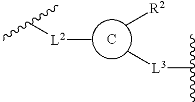

is selected from:

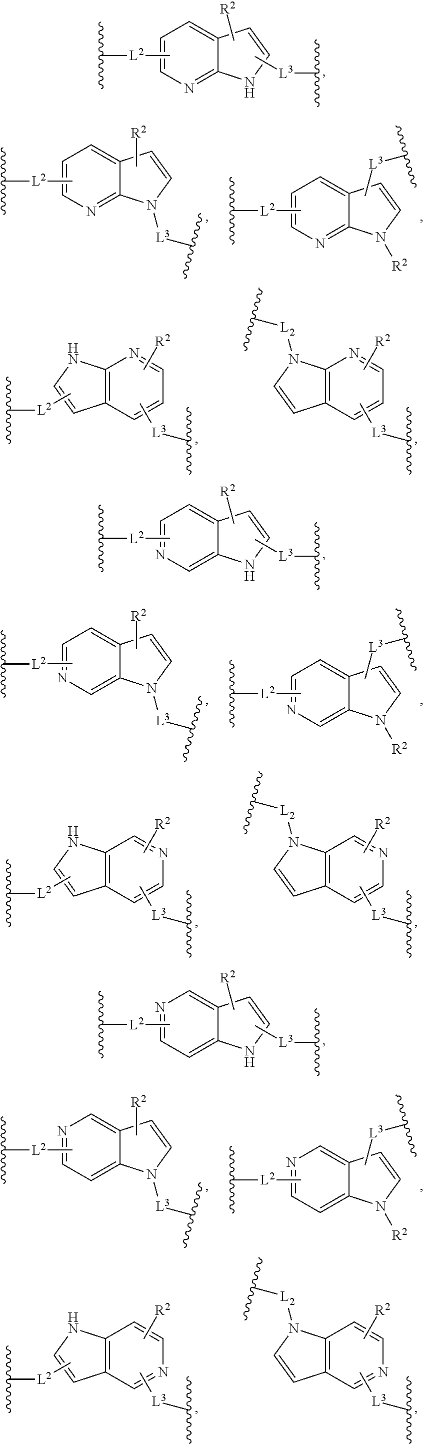

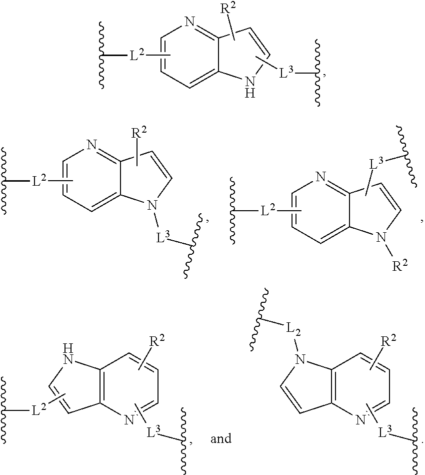

In some embodiments,

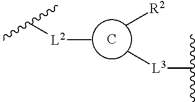

is selected from:

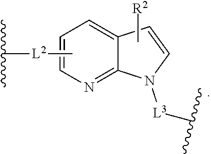

In some embodiments,

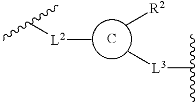

is selected from:

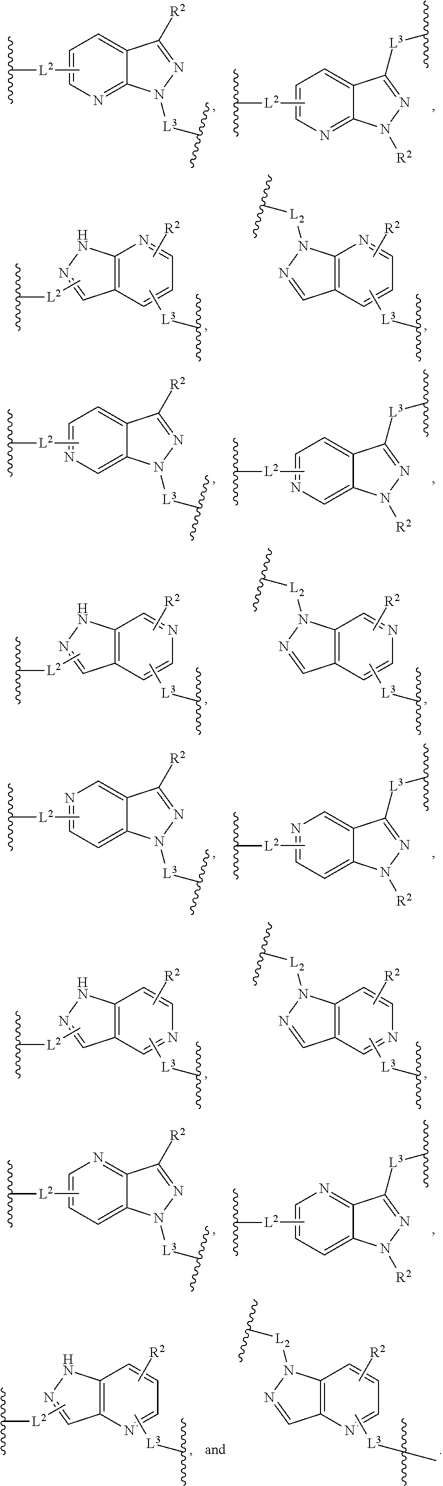

In some embodiments,

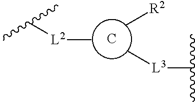

is selected from:

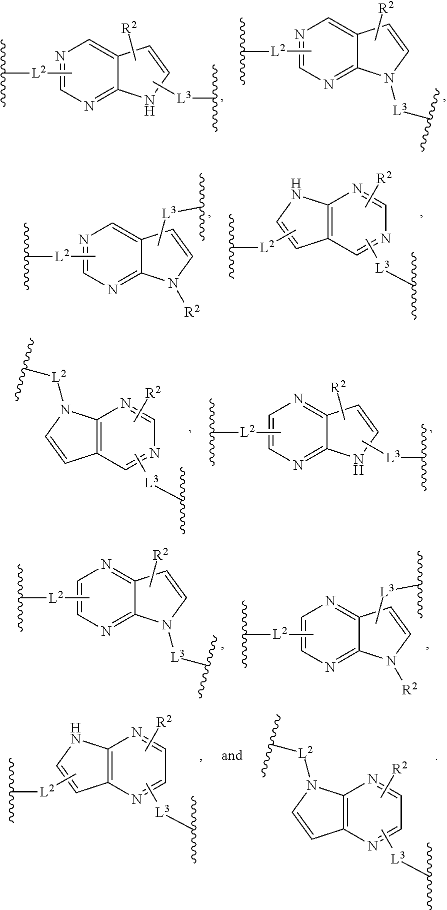

In some embodiments,

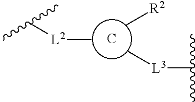

is selected from:

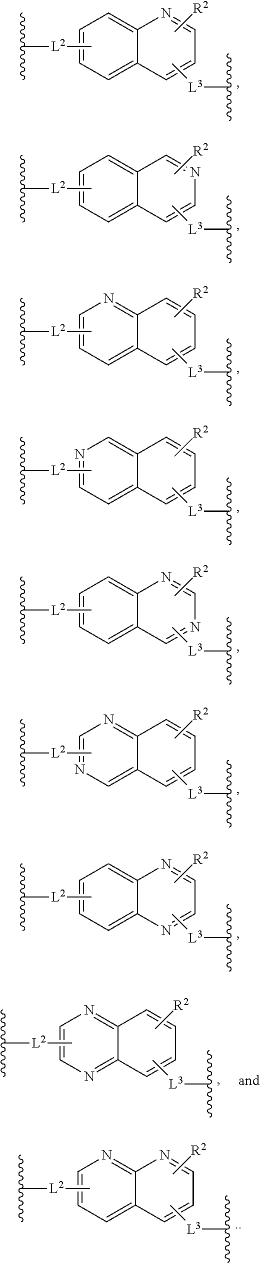

In some embodiments,

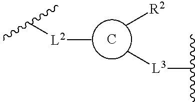

is selected from:

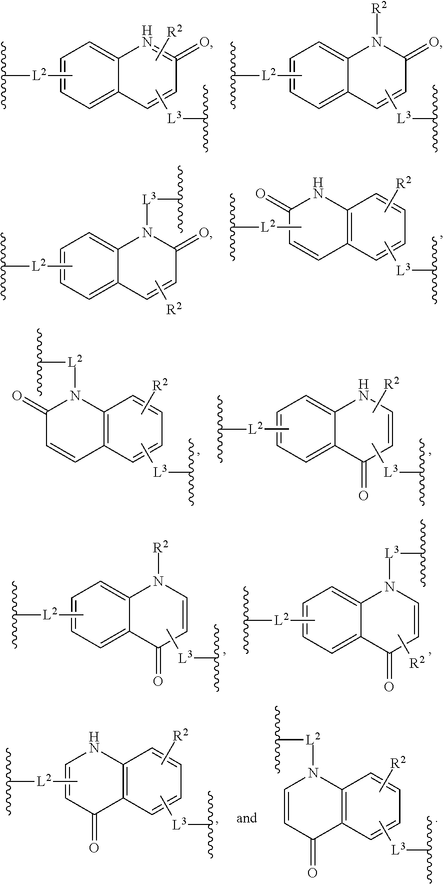

In some embodiments,

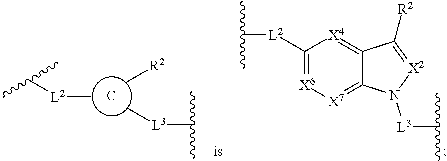

X² is —CH or N; X⁴ is —CH or N; X⁶ is —CH or N; and X⁷ is —CH or N, and wherein at least one X², X⁴, X⁶, or X⁷ is N.

In some embodiments,

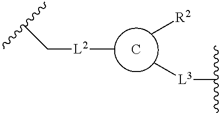

is selected from the following:

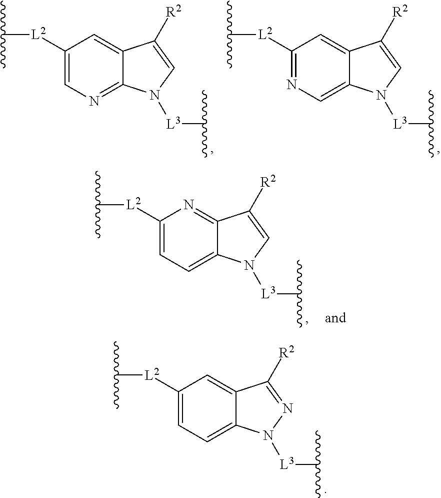

In some embodiments,

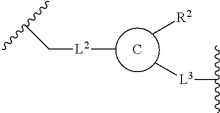

is selected from the following:

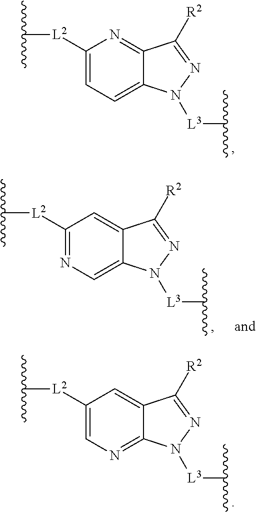

In some embodiments,

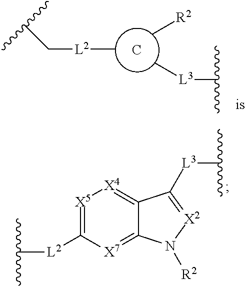

X² is —CH or N; X⁴ is —CH or N; X⁵ is —CH or N; and X⁷ is —CH or N, and wherein at least one X², X⁴, X⁵, or X⁷ is N.

In some embodiments,

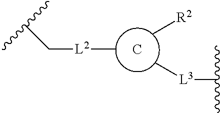

is selected from the following:

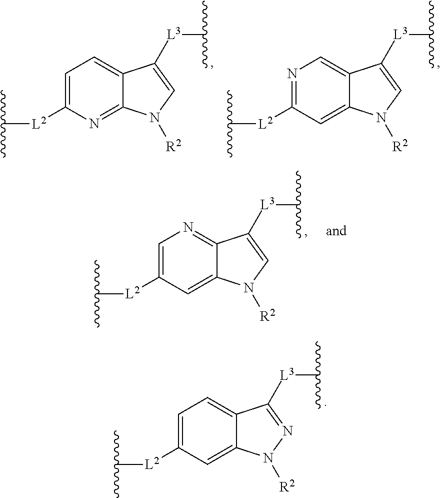

In some embodiments,

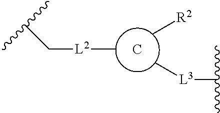

is selected from the following:

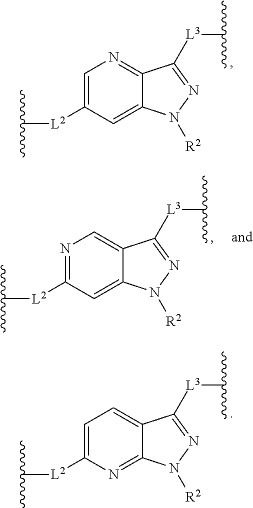

In some embodiments,

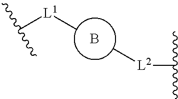

is selected from the following:

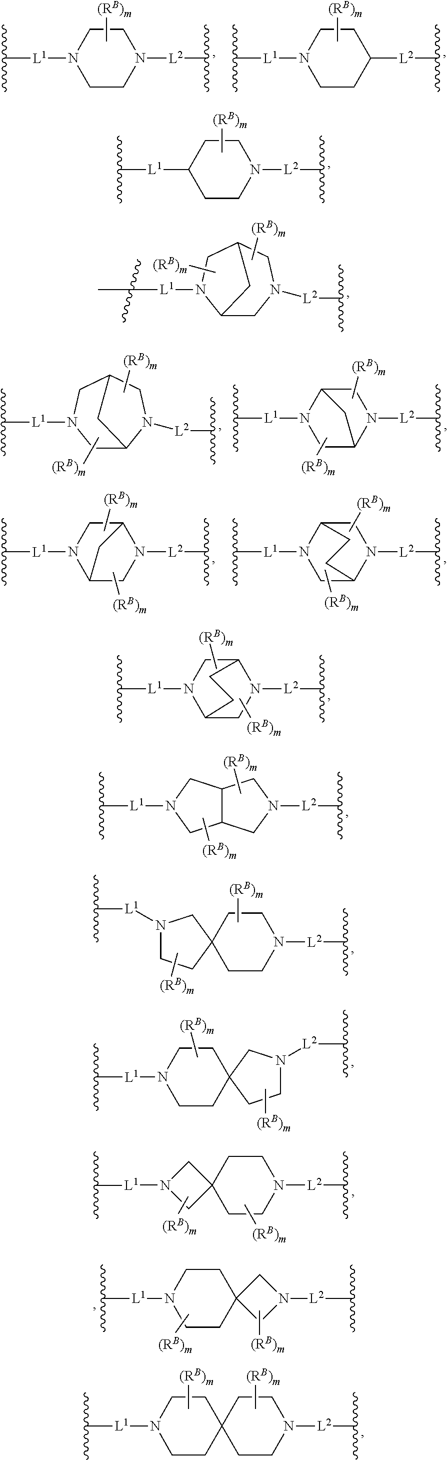

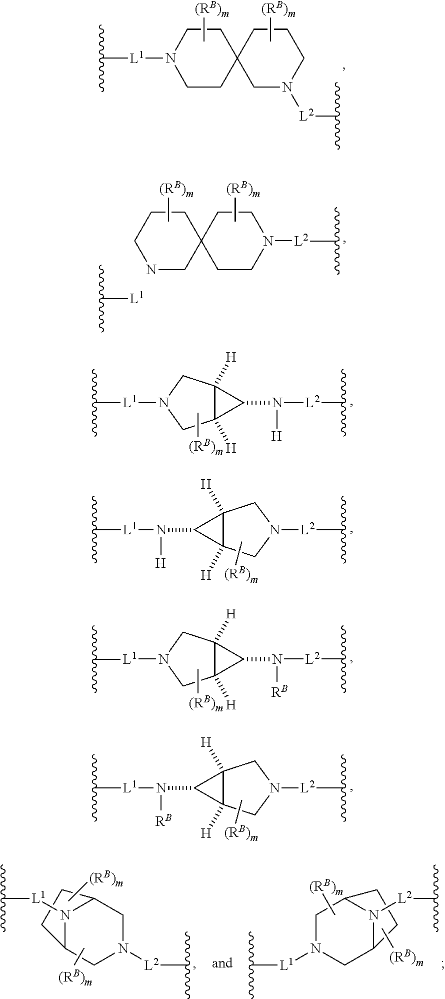

and each m is independently 0, 1, 2, 3, or 4.

In some embodiments,

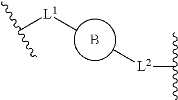

is selected from the following:

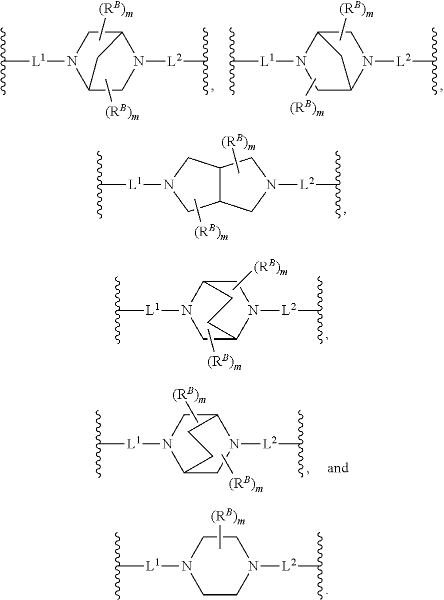

In some embodiments,

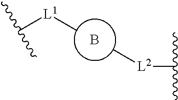

is selected from

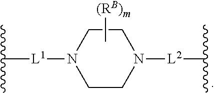

In some embodiments,

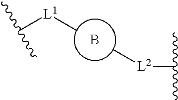

is selected from the following:

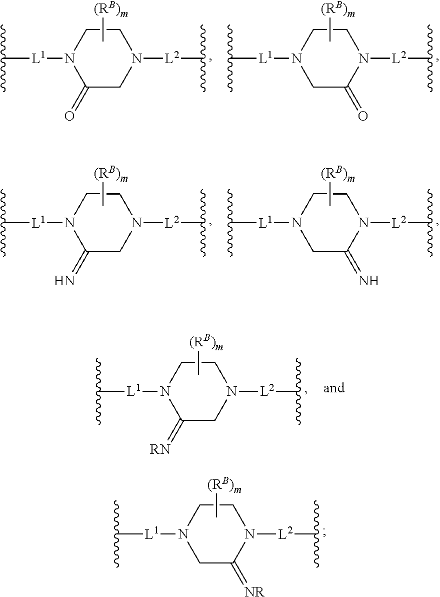

R is optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R is C₁-C₄alkyl.

In some embodiments, a compound of Formula (VIIIa) has the following structure of Formula (VIIIb), or a pharmaceutically acceptable salt, or solvate thereof:

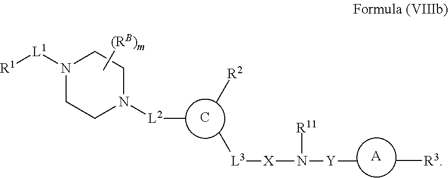

In some embodiments, a compound of Formula (VIIIa) has the following structure of Formula (VIIIc), or a pharmaceutically acceptable salt, or solvate thereof:

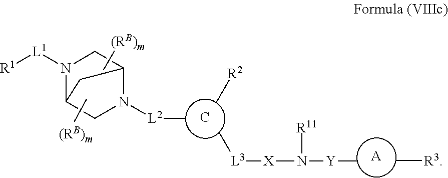

In some embodiments,

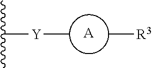

is selected from the following:

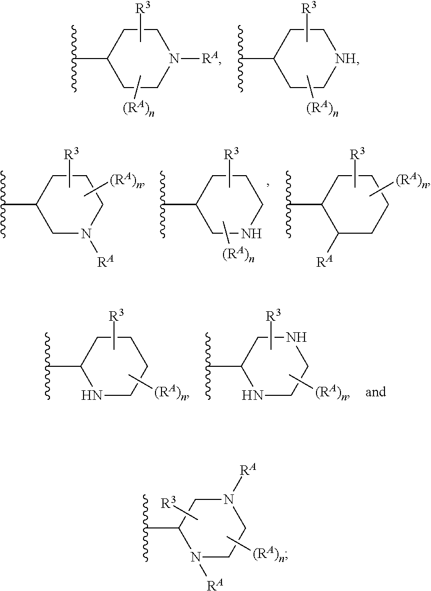

and each n is independently 0, 1, 2, 3, or 4.

In some embodiments,

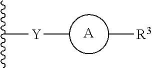

is selected from the following:

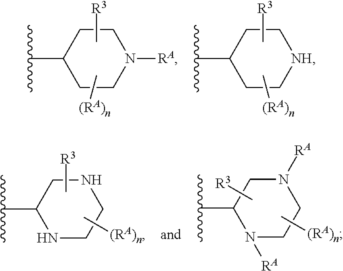

In some embodiments,

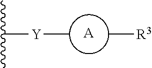

is selected from the following:

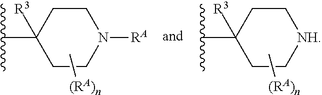

In some embodiments, L¹ is —CH₂—, C(═O), O, S, S(═O), S(═O)₂, or NR⁴. In some embodiments, L¹ is —CH₂—. In some embodiments, L² is —CH₂—, C(═O), O, S, S(═O), S(═O)₂, or NR⁴. In some embodiments, L² is —CH₂—. In some embodiments, In some embodiments, L³ is absent, —CH₂—, —CH₂—CH₂—, or —CH₂—CH₂—CH₂—. In some embodiments, L³ is —CH₂—CH₂—.

In some embodiments, L³ is:

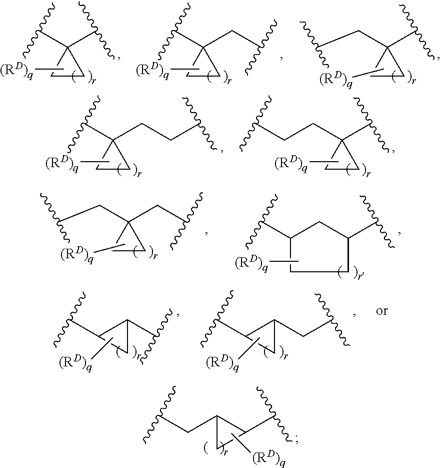

each q is independently 0, 1, 2, 3, or 4;

r is 1, 2, 3, 4, or 5; and

r′ is 1 or 2.

In some embodiments, X is —CH₂— or C(═O). In some embodiments, X is —CH₂—.

In some embodiments, X is:

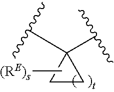

each s is independently 0, 1, 2, 3, or 4; and

t is 1, 2, 3, 4, or 5.

In some embodiments, X is:

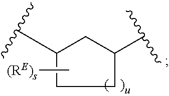

each s is independently 0, 1, 2, 3, or 4; and

u is 0, 1, or 2.

In some embodiments, L³-X is —CH₂—CH₂—CH₂—. In some embodiments, Y is —CH₂— or C(═O). In some embodiments, Y is C(═O). In some embodiments, R¹¹ is hydrogen.

In some embodiments, R³ is H. In some embodiments, R³ is CH₂N(R⁹)(R¹⁰). In some embodiments, R³ is N(R⁹)(R¹⁰).

In some embodiments, R⁹ and R¹⁰ are each H. In some embodiments, R⁹ is H and R¹⁰ is —C₁-C₄alkylene-(optionally substituted phenyl) or —C₁-C₄alkylene-(optionally substituted heteroaryl). In some embodiments, R⁹ is H and R¹⁰ is —CH₂-(optionally substituted phenyl). In some embodiments, R⁹ is H and R¹⁰ is —CH₂-(optionally substituted heteroaryl).

In some embodiments, R³ is CH₂N(R⁹)(R¹⁰); and R⁹ and R¹⁰ are each H. In some embodiments, R³ is CH₂N(R⁹)(R¹⁰); and R⁹ is H and R¹⁰ is —C₁-C₄alkylene-(optionally substituted phenyl) or —C₁-C₄alkylene-(optionally substituted heteroaryl). In some embodiments, R³ is CH₂N(R⁹)(R¹⁰); and R⁹ is H and R¹⁰ is —C₁-C₄alkylene-(optionally substituted phenyl). In some embodiments, R³ is CH₂N(R⁹)(R¹⁰); and R⁹ is H and R¹⁰ is —C₁-C₄alkylene-(optionally substituted heteroaryl). In some embodiments, R³ is N(R⁹)(R¹⁰); and R⁹ and R¹⁰ are each H. In some embodiments, R³ is N(R⁹)(R¹⁰); and R⁹ is H and R¹⁰ is —C₁-C₄alkylene-(optionally substituted phenyl) or —C₁-C₄alkylene-(optionally substituted heteroaryl). In some embodiments, R³ is N(R⁹)(R¹⁰); and R⁹ is H and R¹⁰ is —C₁-C₄alkylene-(optionally substituted phenyl). In some embodiments, R³ is N(R⁹)(R¹⁰); and R⁹ is H and R¹⁰ is —C₁-C₄alkylene-(optionally substituted heteroaryl).

In some embodiments, R¹ is an unsubstituted phenyl. In some embodiments, R¹ is a substituted phenyl. In some embodiments, R¹ is selected from:

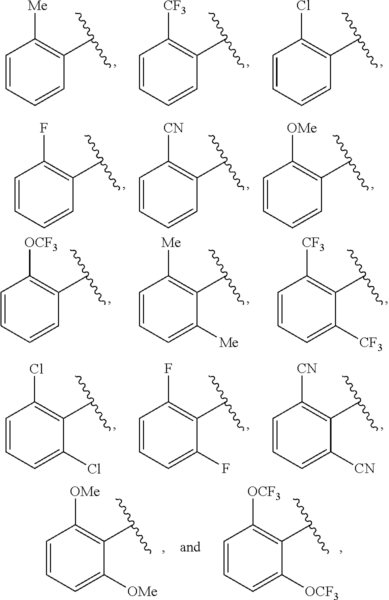

In some embodiments, R² is an unsubstituted phenyl. In some embodiments, R² is a substituted phenyl. In some embodiments, R² is selected from:

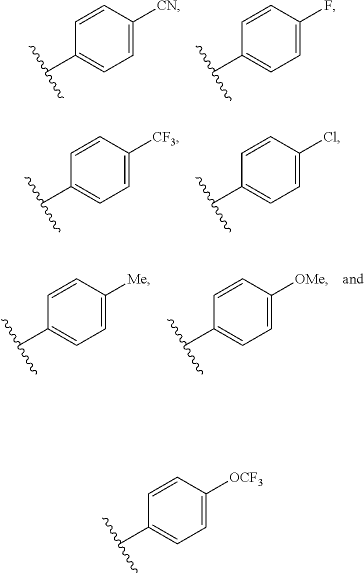

In some embodiments, R² is selected from:

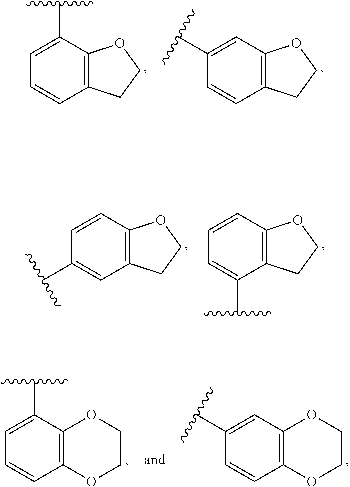

In some embodiments, a compound of Formula (VIIIa) has the following structure of Formula (VIIId), or a pharmaceutically acceptable salt, or solvate thereof:

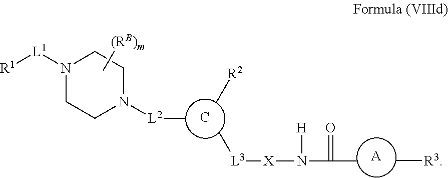

In some embodiments, a compound of Formula (VIIIa) has the following structure of Formula (VIIIe), or a pharmaceutically acceptable salt, or solvate thereof:

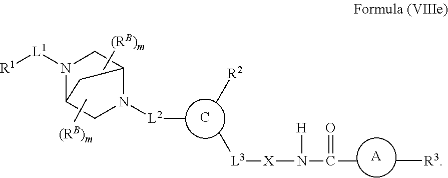

In some embodiments, compounds of Formula (VIIIa) include, but are not limited to, those of Formula (VIIIf) as described in Table A1.

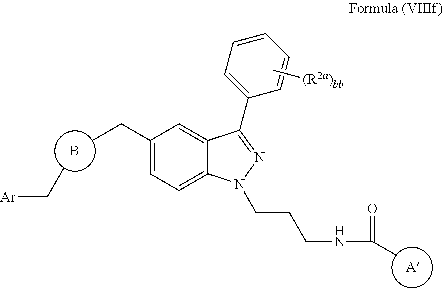

TABLE A1

| Entry | 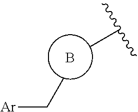 |
|---|---|
| 1 | 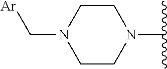 |
| 2 | 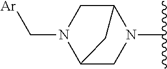 |
| 3 | 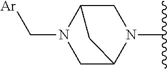 |
| 4 | 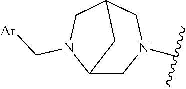 |
| 5 | 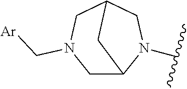 |
| 6 | 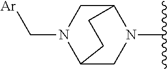 |
| 7 | 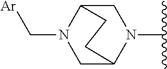 |
| 8 | 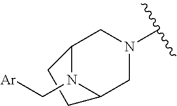 |
| 9 | 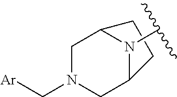 |
| 10 | 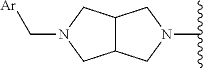 |
| 11 | 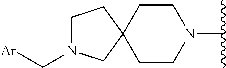 |
| 12 | 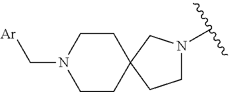 |
| 13 | 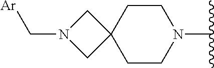 |
| 14 | 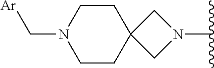 |
| 15 | 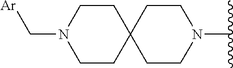 |
| 16 | 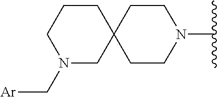 |
| 17 | 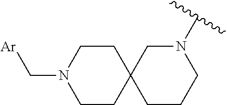 |
| 18 | 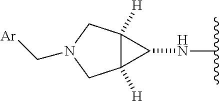 |
| 19 | 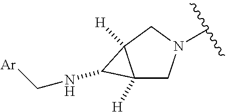 |

For compounds of Formula (VIIIf), each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, bb is 0. In some embodiments bb is 1. In some embodiments, R^{2a} is halogen and bb is 1. In some embodiments, R^{2a} is —OCF₃ and bb is 1.

In some embodiments, Ar is unsubstituted phenyl. In some embodiments, Ar is a substituted phenyl. In some embodiments Ar is selected from:

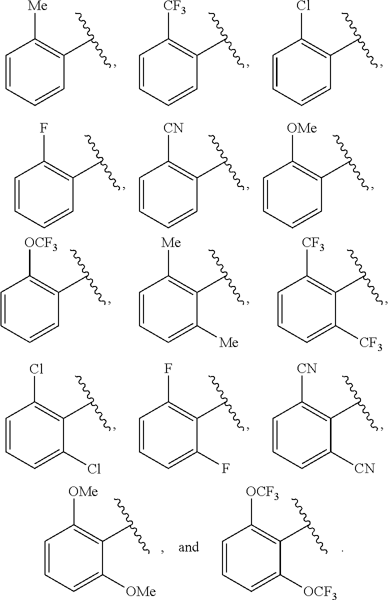

In some embodiments,

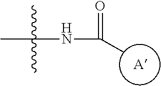

is selected from

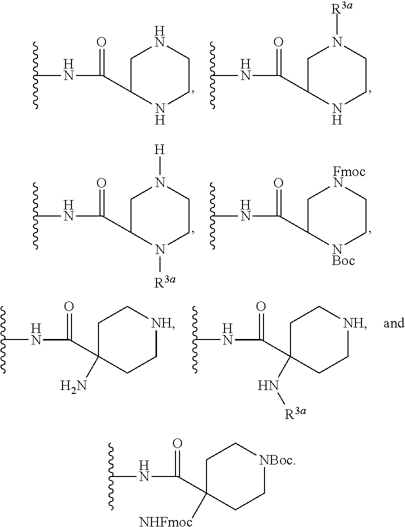

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

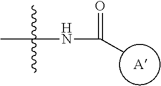

is selected from

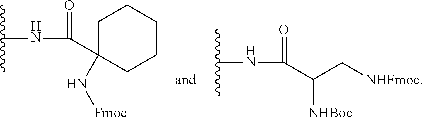

In some embodiments, compounds of Formula (VIIIa) include, but are not limited to, those of Formula (VIIIg) as described in Table A2.

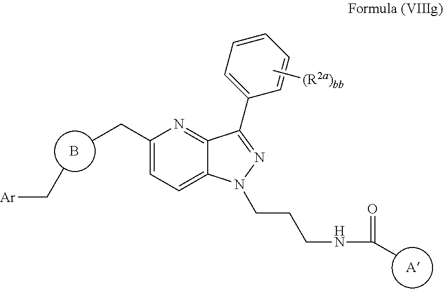

TABLE A2

| Entry | 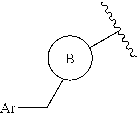 |
|---|---|
| 1 | 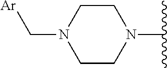 |
| 2 | 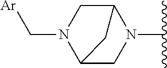 |
| 3 | 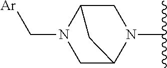 |
| 4 | 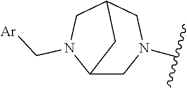 |
| 5 | 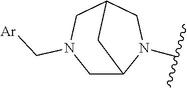 |
| 6 | 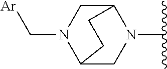 |
| 7 | 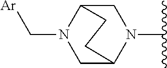 |
| 8 | 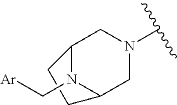 |
| 9 | 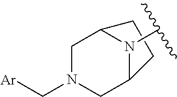 |
| 10 | 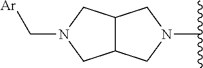 |
| 11 | 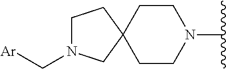 |
| 12 | 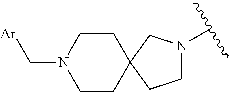 |
| 13 | 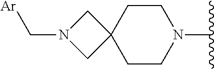 |
| 14 | 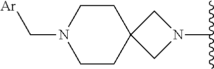 |
| 15 | 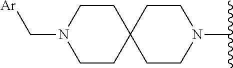 |
| 16 | 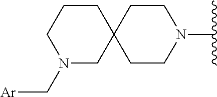 |
| 17 | 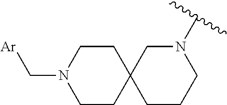 |
| 18 | 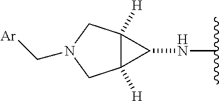 |
| 19 | 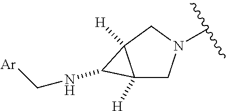 |

For compounds of Formula (VIIIg), each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, bb is 0. In some embodiments bb is 1. In some embodiments, R^{2a} is halogen and bb is 1. In some embodiments, R^{2a} is —OCF₃ and bb is 1. In some embodiments, Ar is unsubstituted phenyl. In some embodiments, Ar is a substituted phenyl. In some embodiments Ar is selected from:

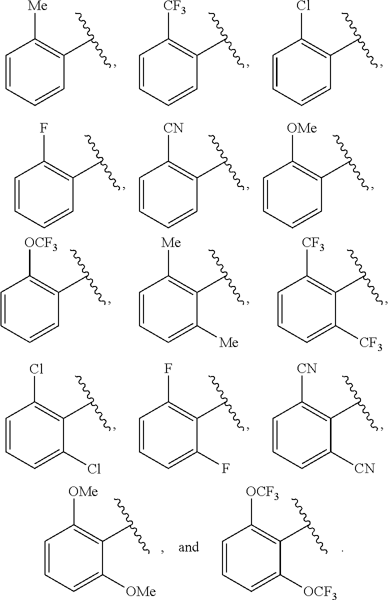

In some embodiments,

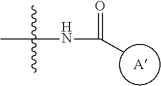

is selected from

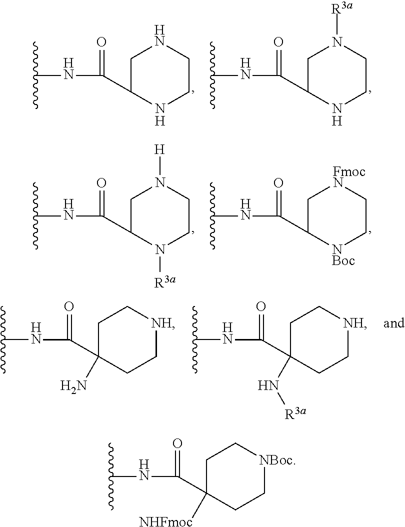

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

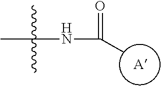

is selected from

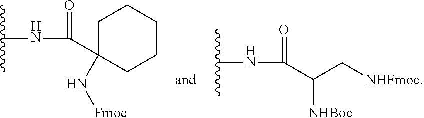

In some embodiments, compounds of Formula (VIIIa) include, but are not limited to, those of Formula (VIIIh) as described in Table A3.

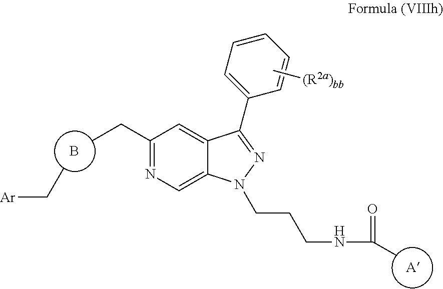

TABLE A3

| Entry | 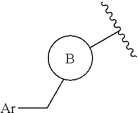 |
|---|---|
| 1 | 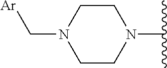 |
| 2 | 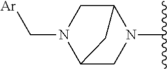 |
| 3 | 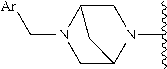 |
| 4 | 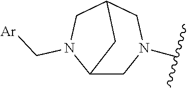 |
| 5 | 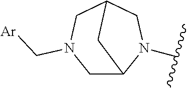 |
| 6 | 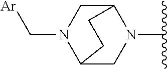 |
| 7 | 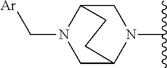 |
| 8 | 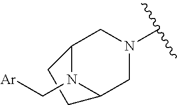 |
| 9 | 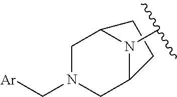 |
| 10 | 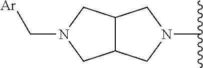 |
| 11 | 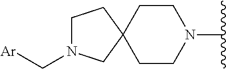 |
| 12 | 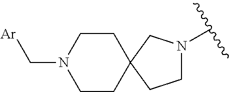 |
| 13 | 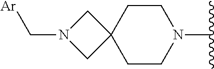 |
| 14 | 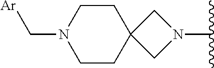 |
| 15 | 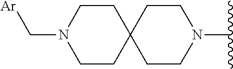 |
| 16 | 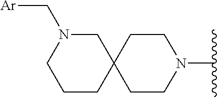 |
| 17 | 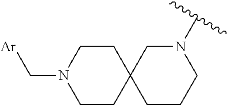 |
| 18 | 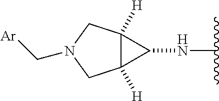 |
| 19 | 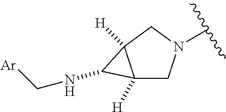 |

For compounds of Formula (VIIIh), each R2a is independently H, CN, CF3, halogen, —OH, —O—C1-C6alkyl, —OCF3, —SH, —S—C1-C6alkyl, —NH2, —NH(C1-C6alkyl), —N(C1-C6alkyl)2, optionally substituted C1-C6alkyl, optionally substituted C3-C6cycloalkyl, optionally substituted C1-C6heteroalkyl, optionally substituted C2-C10heterocycloalkyl, optionally substituted aryl, or optionally substituted heteroaryl; each bb is 0, 1, or 2; and Ar is substituted or unsubstituted phenyl. In some embodiments, R2a is halogen. In some embodiments, R2a is —OCF3. In some embodiments, bb is 0. In some embodiments bb is 1. In some embodiments, R2a is halogen and bb is 1. In some embodiments, R2a is —OCF3 and bb is 1. In some embodiments, Ar is unsubstituted phenyl. In some embodiments, Ar is a substituted phenyl.

In some embodiments Ar is selected from:

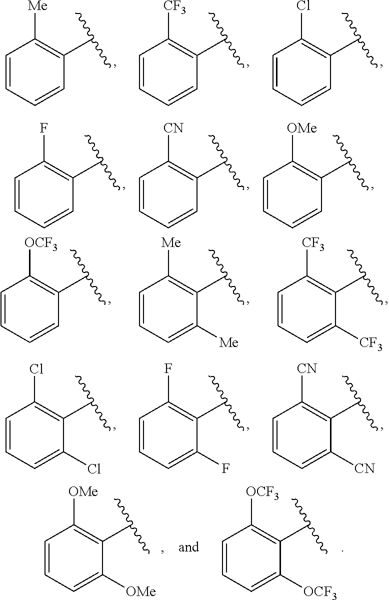

In some embodiments,

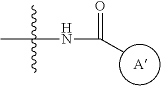

is selected from

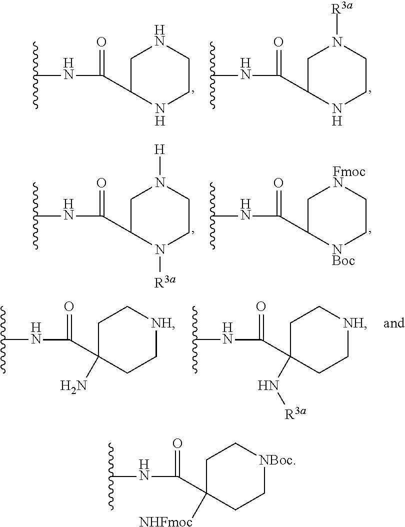

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

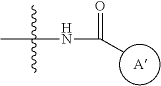

is selected from

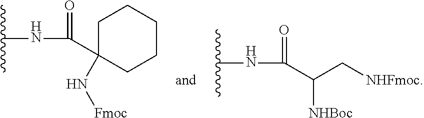

In some embodiments, compounds of Formula (VIIIa) include, but are not limited to, those of Formula (VIIIi) as described in Table A4.

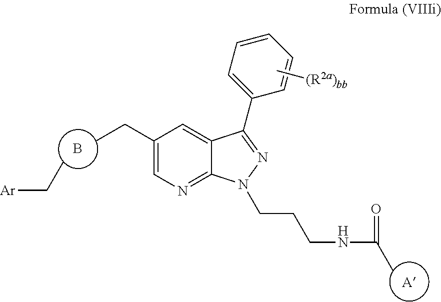

TABLE A4

| Entry | 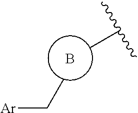 |
|---|---|
| 1 | 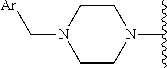 |
| 2 | 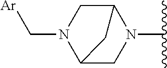 |
| 3 | 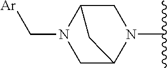 |
| 4 | 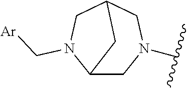 |
| 5 | 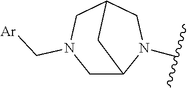 |
| 6 | 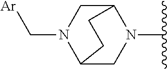 |
| 7 | 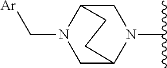 |
| 8 | 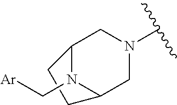 |
| 9 | 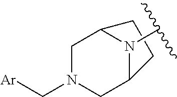 |
| 10 | 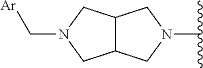 |
| 11 | 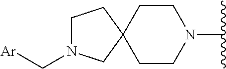 |
| 12 | 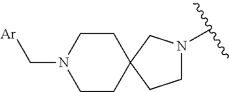 |
| 13 | 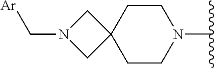 |
| 14 | 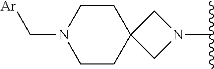 |
| 15 | 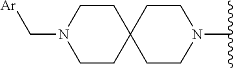 |
| 16 | 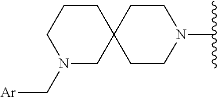 |
| 17 | 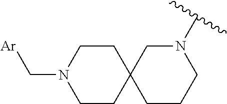 |
| 18 | 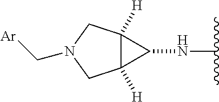 |
| 19 | 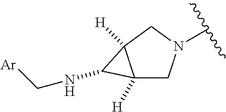 |

For compounds of Formula (VIIIi), each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, bb is 0. In some embodiments bb is 1. In some embodiments, R^{2a} is halogen and bb is 1. In some embodiments, R^{2a} is —OCF₃ and bb is 1. In some embodiments, Ar is unsubstituted phenyl.

In some embodiments, Ar is a substituted phenyl. In some embodiments Ar is selected from:

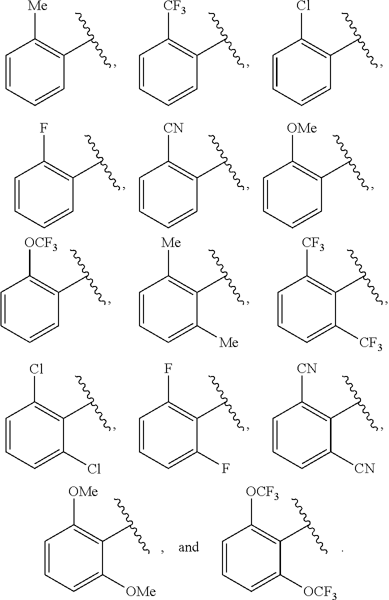

In some embodiments,

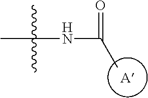 [SMILES: *NC(C)=O]

is selected from

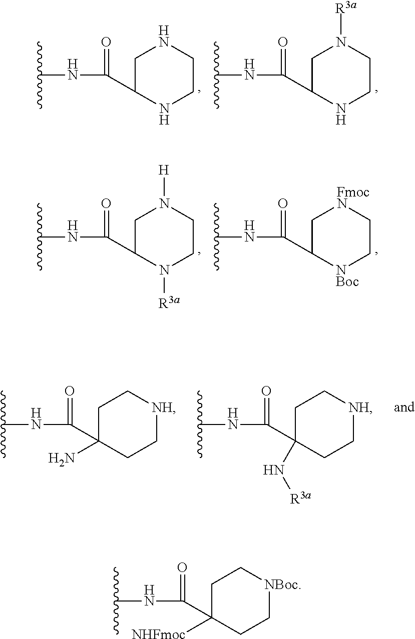

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

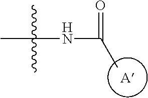 [SMILES: *NC(C)=O]

is selected from

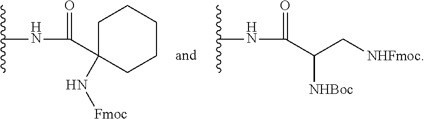

In some embodiments, compounds of Formula (VIIIa) include, but are not limited to, those of Formula (VIIIj) as described in Table A5.

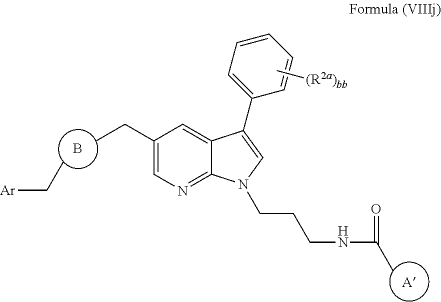

TABLE A5

| Entry | 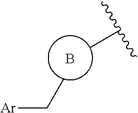 |
|---|---|
| 1 | 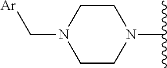 |
| 2 | 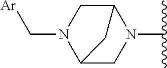 |
| 3 | 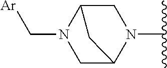 |
| 4 | 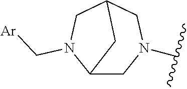 |
| 5 | 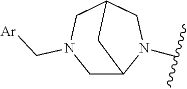 |
| 6 | 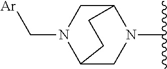 |
| 7 | 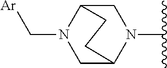 |
| 8 | 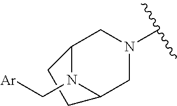 |
| 9 | 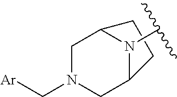 |
| 10 | 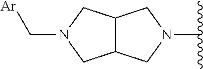 |
| 11 | 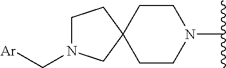 |
| 12 | 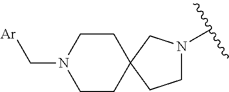 |
| 13 | 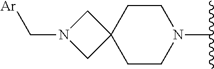 |
| 14 | 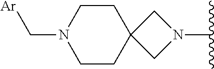 |
| 15 | 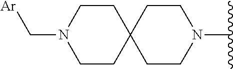 |
| 16 | 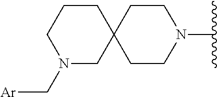 |
| 17 | 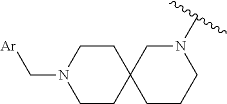 |
| 18 | 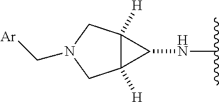 |
| 19 | 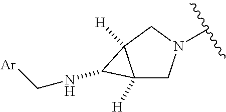 |

For compounds of Formula (VIIIj), each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, bb is 0. In some embodiments bb is 1. In some embodiments, R^{2a} is halogen and bb is 1. In some embodiments, R^{2a} is —OCF₃ and bb is 1. In some embodiments, Ar is unsubstituted phenyl. In some embodiments, Ar is a substituted phenyl. In some embodiments Ar is selected from:

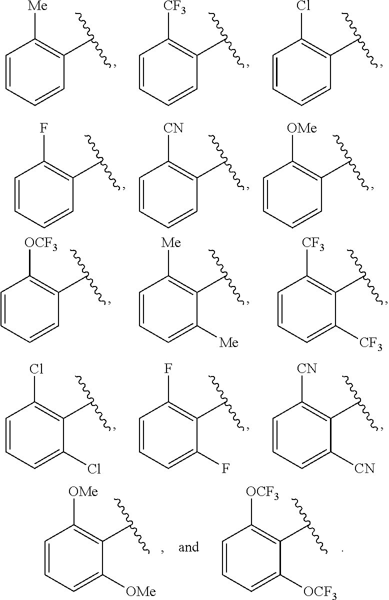

In some embodiments,

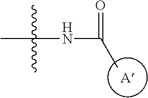 [SMILES: *NC(C)=O]

is selected from

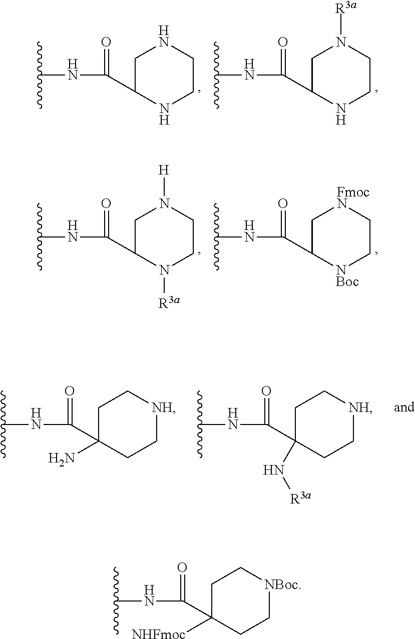

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

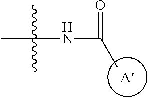 [SMILES: *NC(C)=O]

is selected from

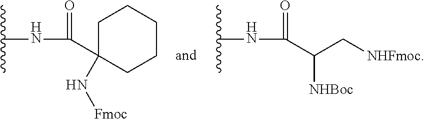

In some embodiments, compounds of Formula (VIIIa) include, but are not limited to, those of Formula (VIIIk) as described in Table A6.

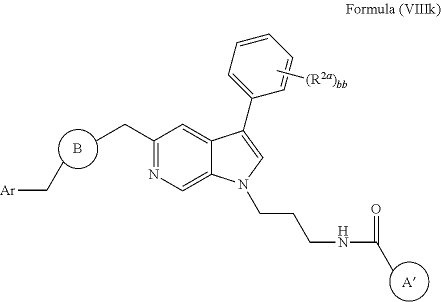

TABLE A6

| Entry | 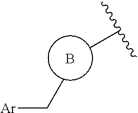 |
|---|---|
| 1 | 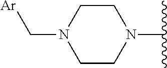 |
| 2 | 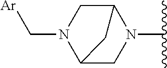 |
| 3 | 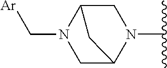 |
| 4 | 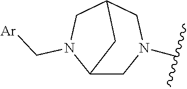 |
| 5 | 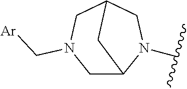 |
| 6 | 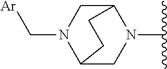 |
| 7 | 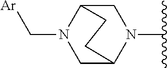 |
| 8 | 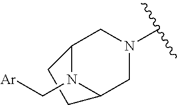 |
| 9 | 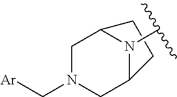 |
| 10 | 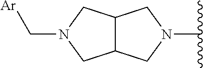 |
| 11 | 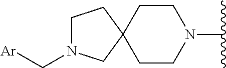 |
| 12 | 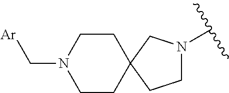 |
| 13 | 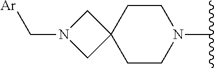 |
| 14 | 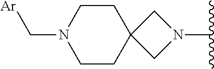 |
| 15 | 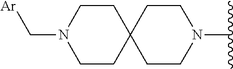 |
| 16 | 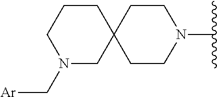 |
| 17 | 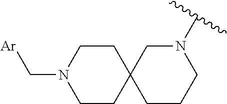 |
| 18 | 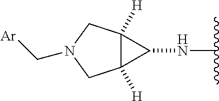 |
| 19 | 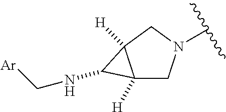 |

For compounds of Formula (VIIIk), each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, bb is 0. In some embodiments bb is 1. In some embodiments, R^{2a} is halogen and bb is 1. In some embodiments, R^{2a} is —OCF₃ and bb is 1. In some embodiments, Ar is unsubstituted phenyl. In some embodiments, Ar is a substituted phenyl. In some embodiments Ar is selected from:

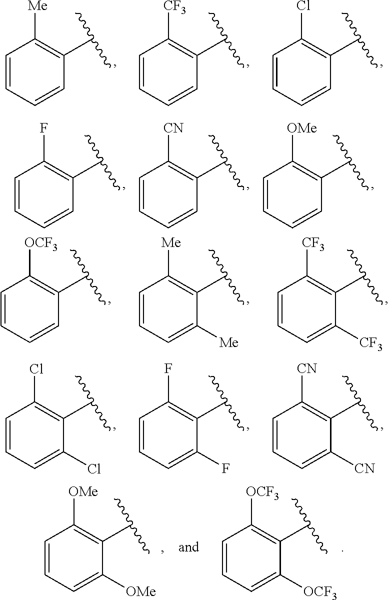

In some embodiments,

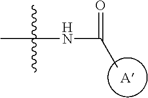 [SMILES: *NC(C)=O]

is selected from

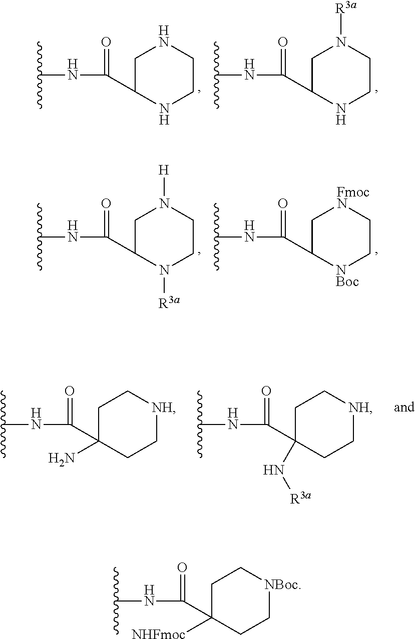

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

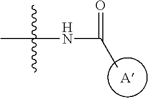 [SMILES: *NC(C)=O]

is selected from

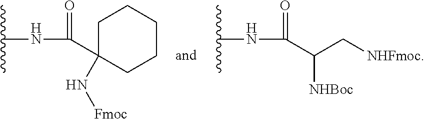

In some embodiments, compounds of Formula (VIIIa) include, but are not limited to, those of Formula (VIIIIl) as described in Table A7.

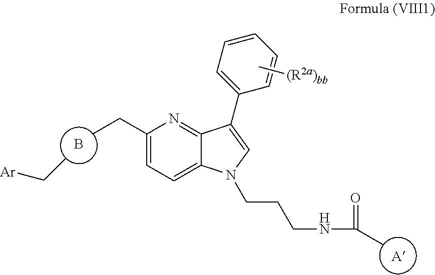

TABLE A7

| Entry | 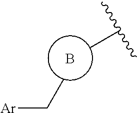 |
|---|---|
| 1 | 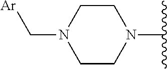 |
| 2 | 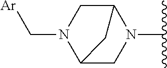 |
| 3 | 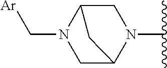 |
| 4 | 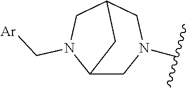 |
| 5 | 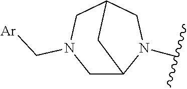 |
| 6 | 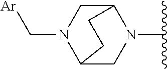 |
| 7 | 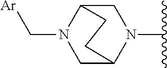 |
| 8 | 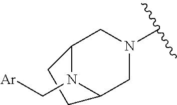 |
| 9 | 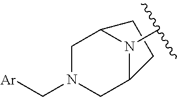 |
| 10 | 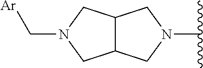 |
| 11 | 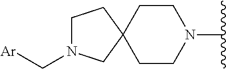 |
| 12 | 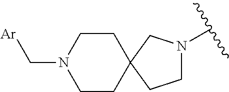 |
| 13 | 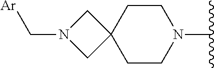 |
| 14 | 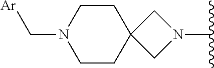 |
| 15 | 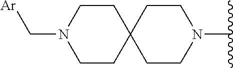 |
| 16 | 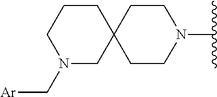 |
| 17 | 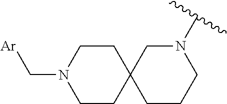 |
| 18 | 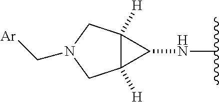 |
| 19 | 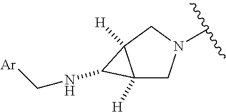 |

For compounds of Formula (VIIIl), each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, bb is 0. In some embodiments bb is 1. In some embodiments, R^{2a} is halogen and bb is 1. In some embodiments, R^{2a} is —OCF₃ and bb is 1. In some embodiments, Ar is unsubstituted phenyl. In some embodiments, Ar is a substituted phenyl. In some embodiments Ar is selected from:

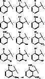

In some embodiments,

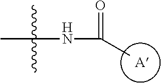

is selected from

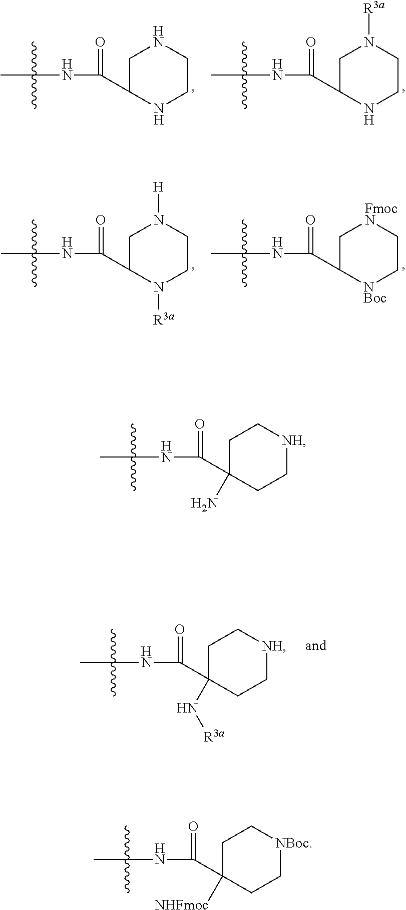

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

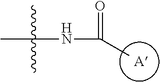

is selected from

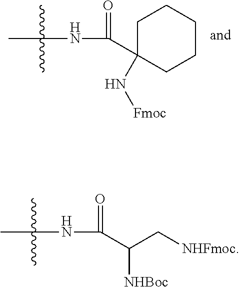

In some embodiments, compounds of Formula (VIIIa) include, but are not limited to, those of Formula (VIIIm) as described in Table A8.

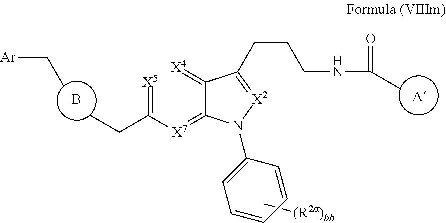

TABLE A8

| Entry | 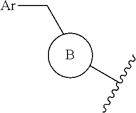 |
|---|---|
| 1 | 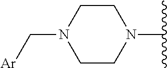 |
| 2 | 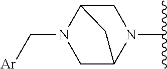 |
| 3 | 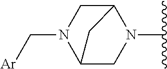 |
| 4 | 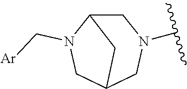 |
| 5 | 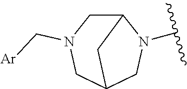 |
| 6 | 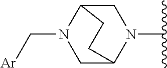 |
| 7 | 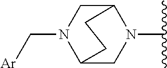 |
| 8 | 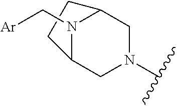 |
| 9 | 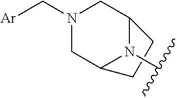 |
| 10 | 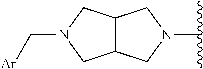 |
| 11 | 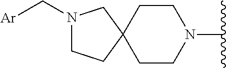 |
| 12 | 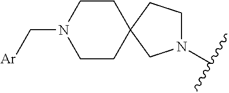 |
| 13 | 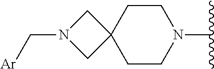 |
| 14 | 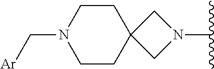 |
| 15 | 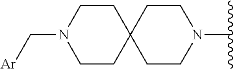 |
| 16 | 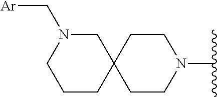 |
| 17 | 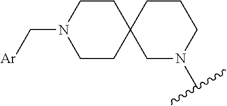 |
| 18 | 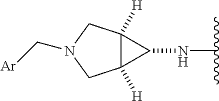 |
| 19 | 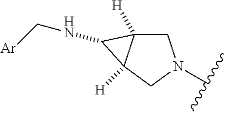 |

For compounds of Formula (VIIIm), X² is —CH or N; X⁴ is —CH or N; X⁵ is —CH or N; and X⁷ is —CH or N, and wherein at least one X², X⁴, X⁵, or X⁷ is N. In some embodiments,

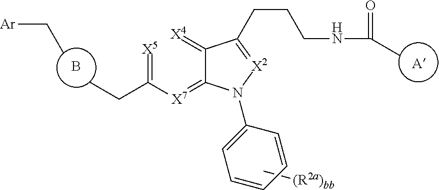

is selected from:

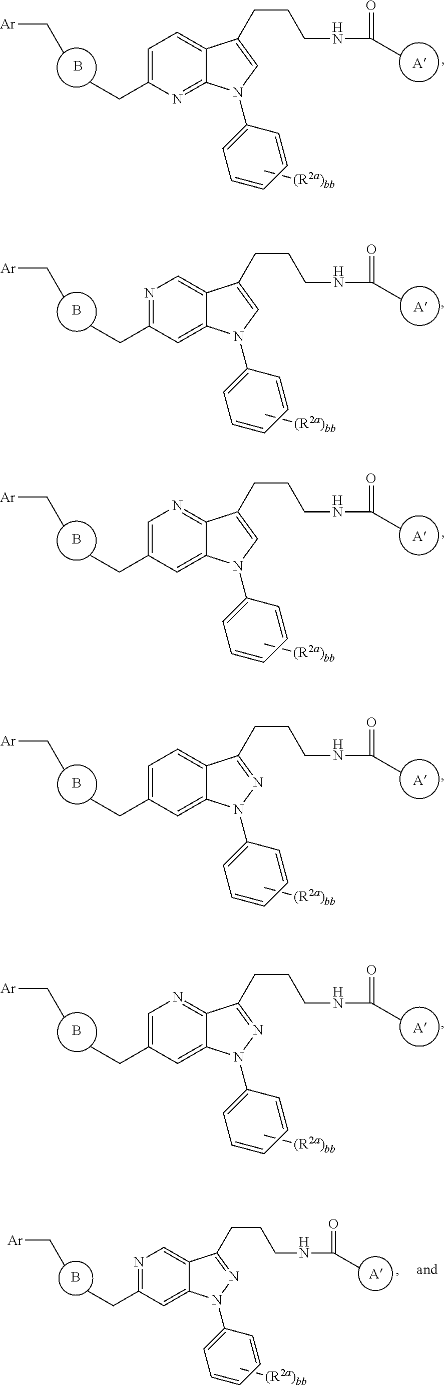

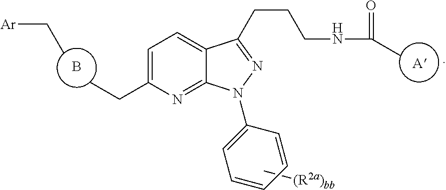

For compounds of Formula (VIIIm), each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2; and Ar is substituted or unsubstituted phenyl. In some embodiments, R^{2a} is halogen. In some embodiments, R^{2a} is —OCF₃. In some embodiments, bb is 0. In some embodiments bb is 1. In some embodiments, R^{2a} is halogen and bb is 1. In some embodiments, R^{2a} is —OCF₃ and bb is 1. In some embodiments, Ar is unsubstituted phenyl. In some embodiments, Ar is a substituted phenyl.

In some embodiments Ar is selected from:

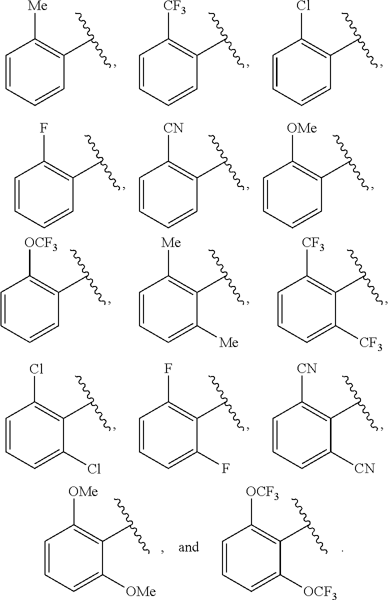

In some embodiments,

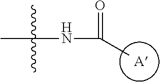

is selected from

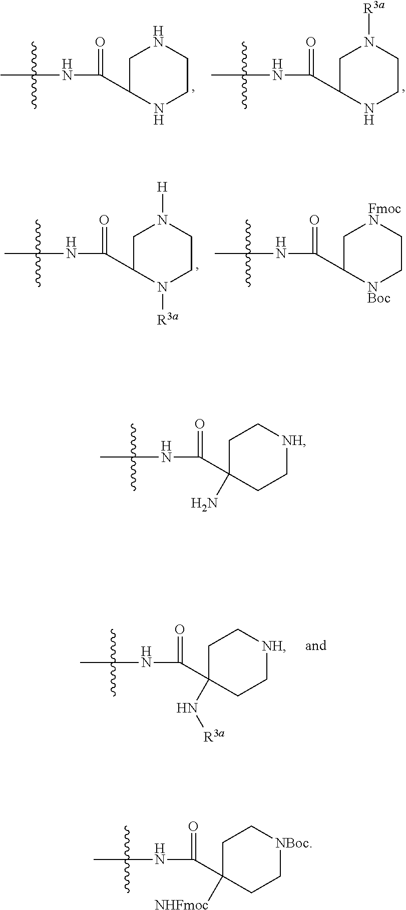

In some embodiments, R^{3a} is optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl). In some embodiments, R^{3a} is —CH₂-(optionally substituted aryl). In some embodiments, R^{3a} is —CH₂-(optionally substituted heteroaryl).

In some embodiments,

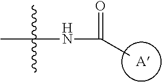

is selected from

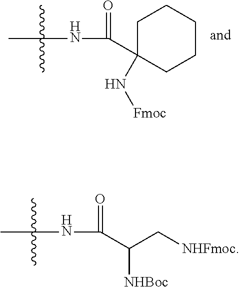

Also provided herein is compound of Formula (IXa), or a pharmaceutically acceptable salt, or solvate thereof:

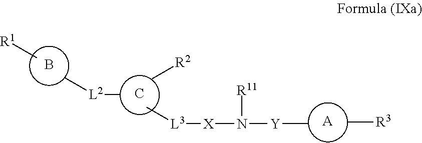

wherein,

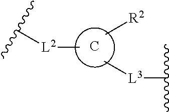

is a bicyclic heteroaryl or heterocycloalkyl that is selected from the following structures:

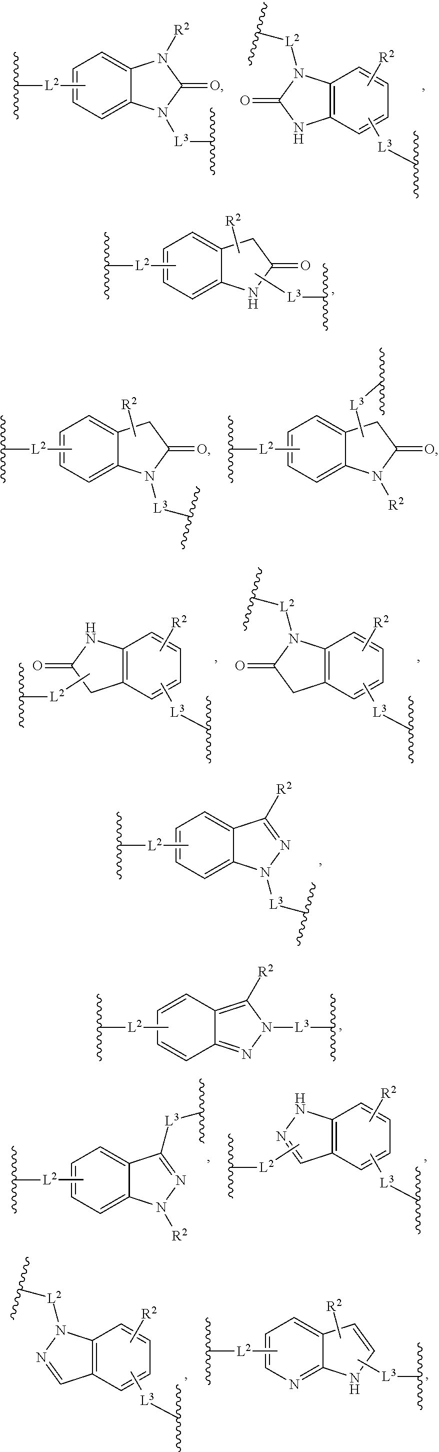

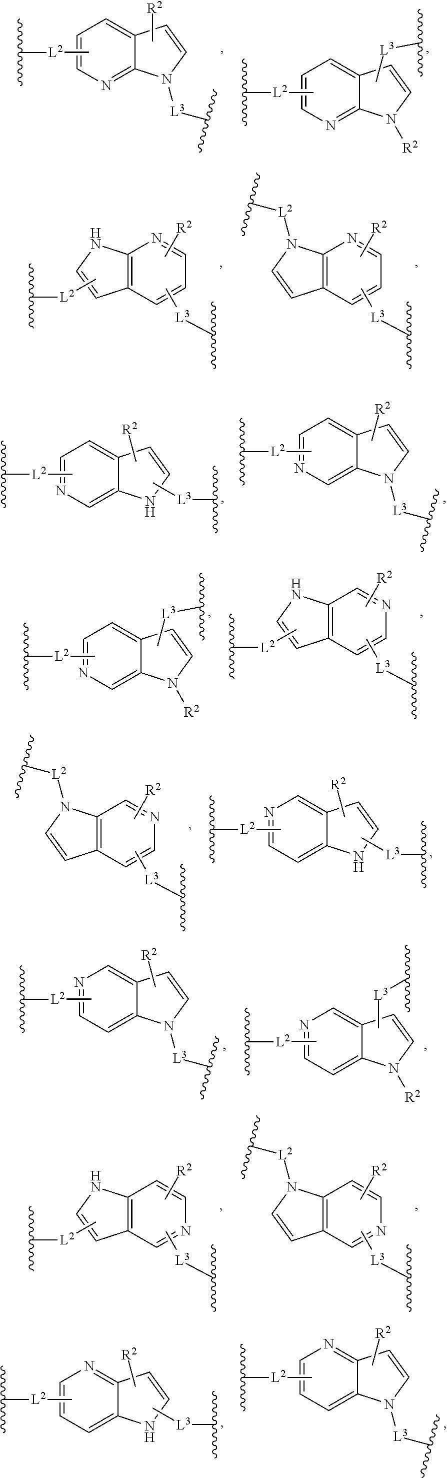

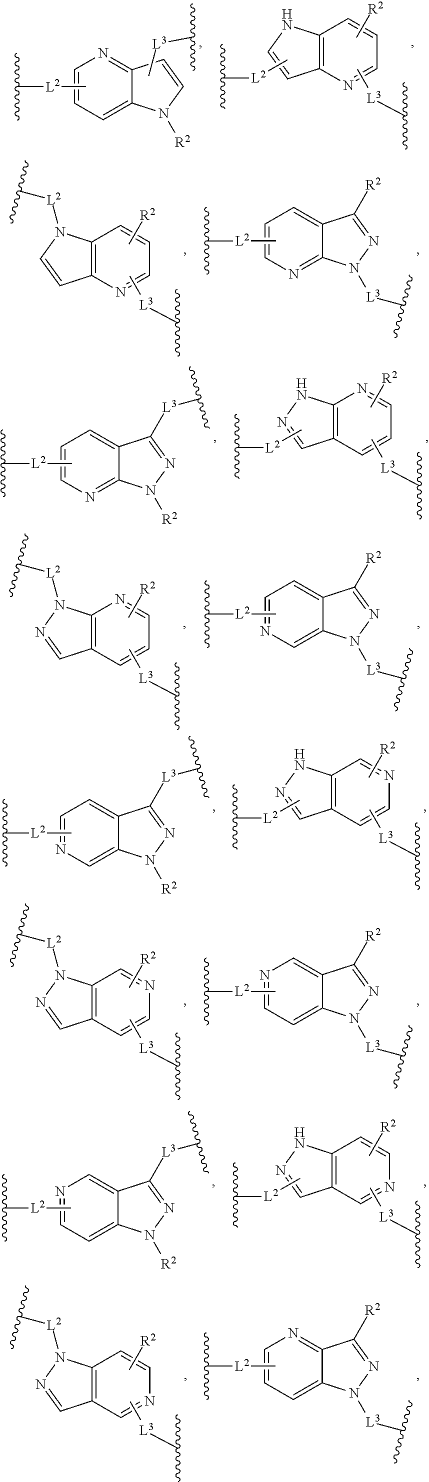

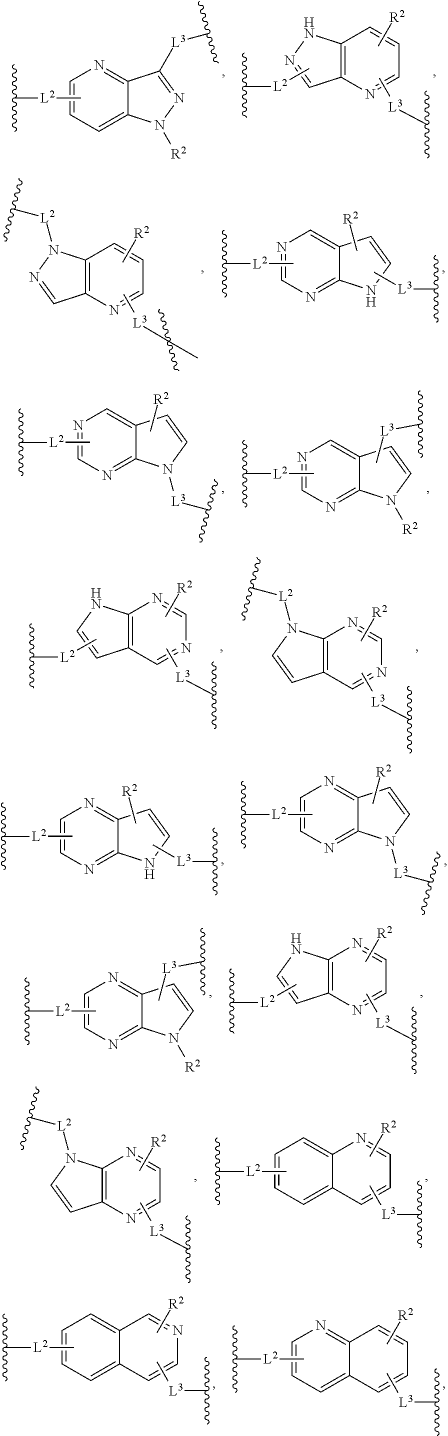

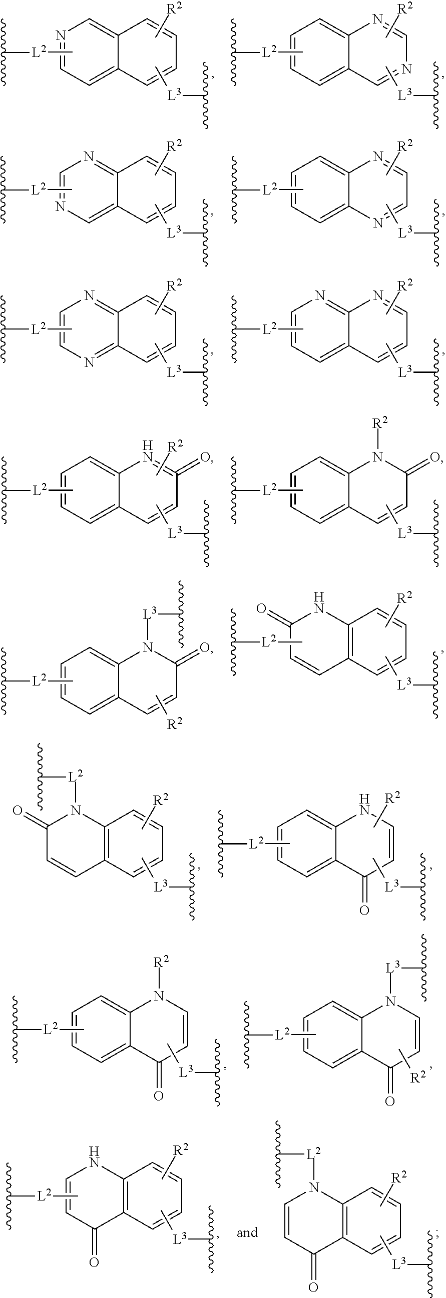

L² is an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N;
wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted C₃-C₆cycloalkylene; an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)— or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N; wherein if Ring A is substituted, then Ring A is substituted with at least one R^{A};
each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), and optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring, and
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring,
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl).

In some embodiments,

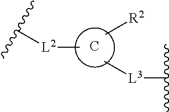

is selected from:

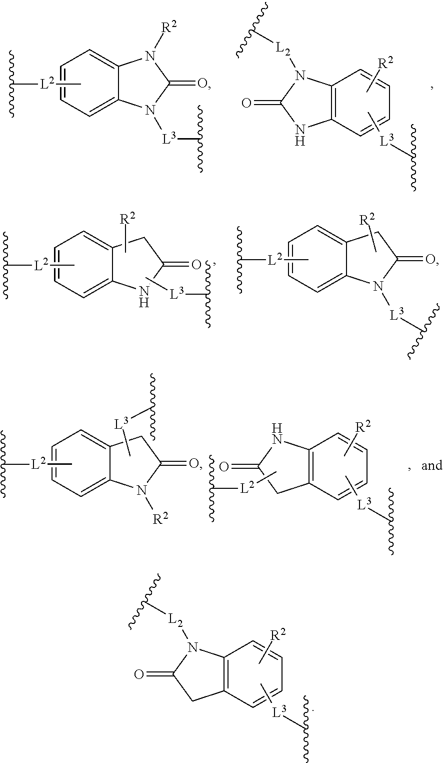

In some embodiments,

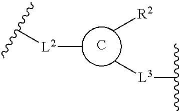

is selected from:

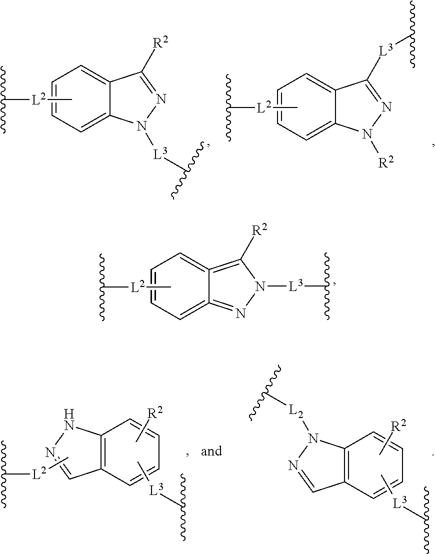

In some embodiments,

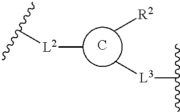

is selected from:

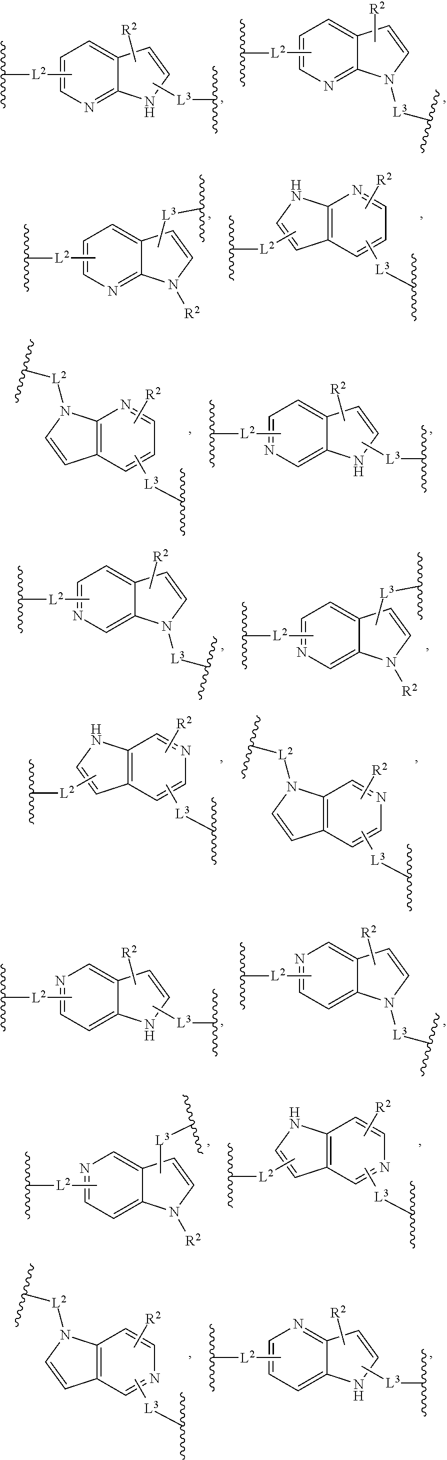

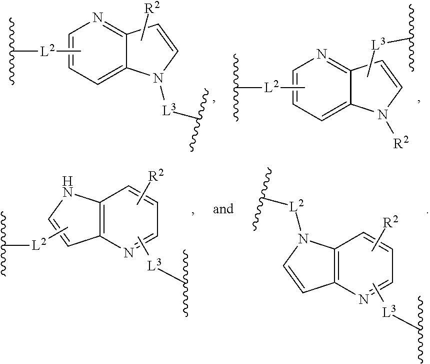

In some embodiments,

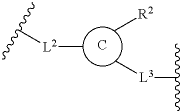

is selected from

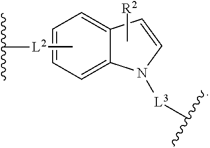

In some embodiments,

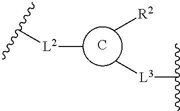

is selected from:

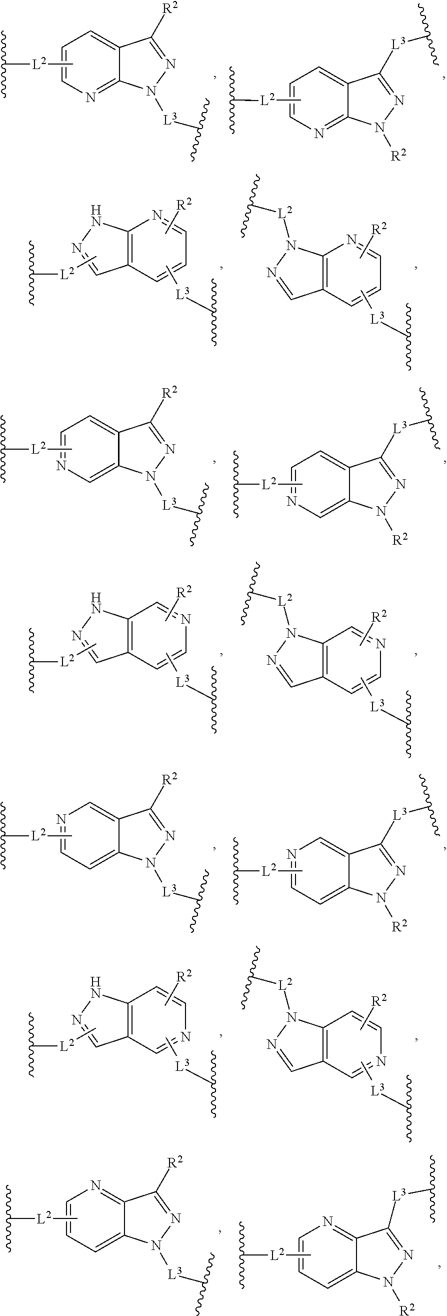

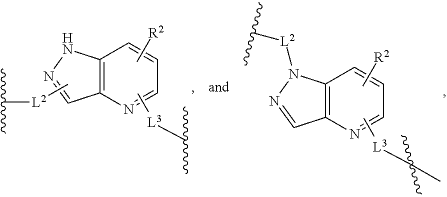

In some embodiments,

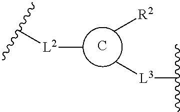

is selected from:

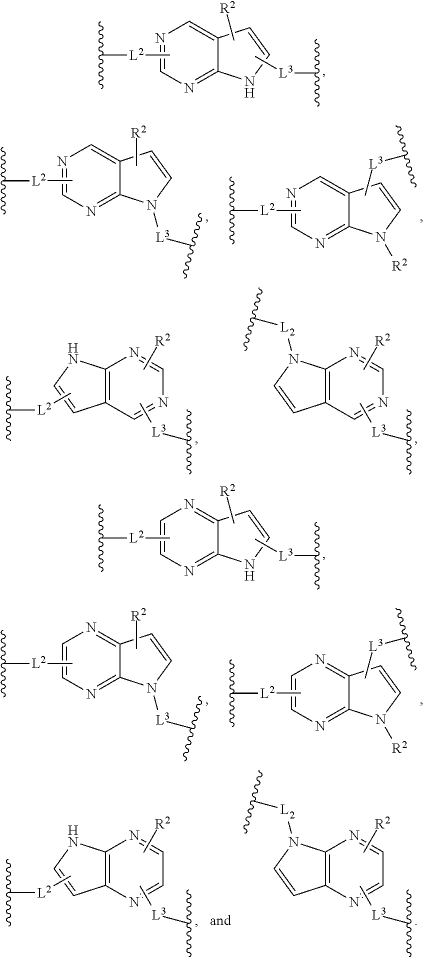

In some embodiments,

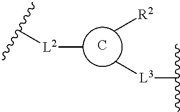

is selected from:

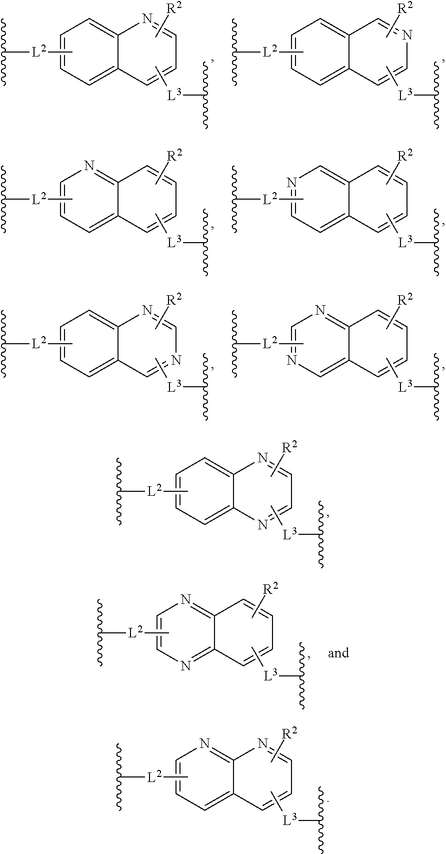

In some embodiments,

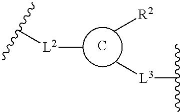

is selected from:

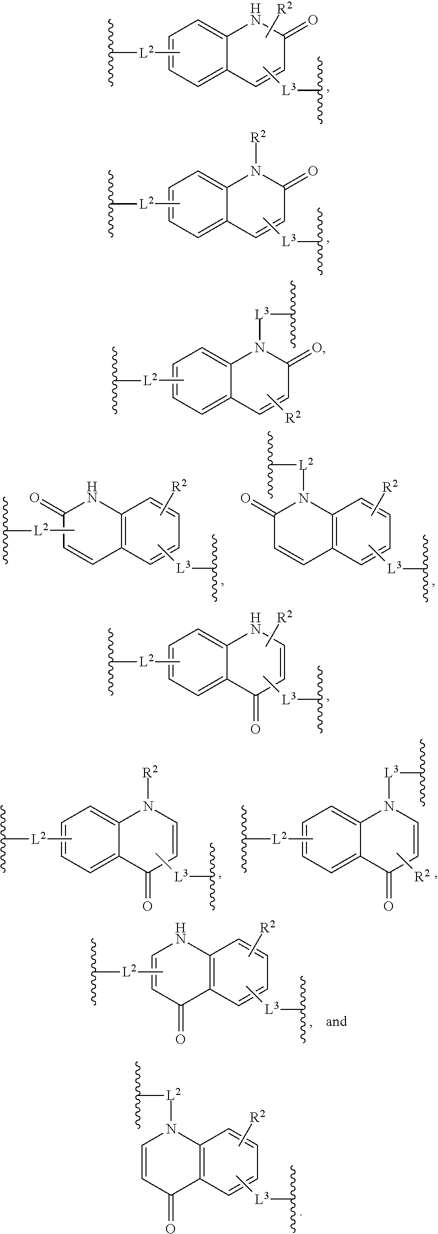

In some embodiments,

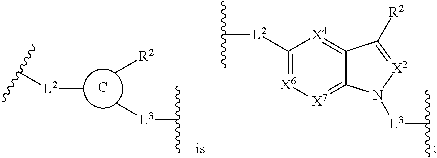

X² is —CH or N; X⁴ is —CH or N; X⁶ is —CH or N; and X⁷ is —CH or N, and wherein at least one X², X⁴, X⁶, or X⁷ is N.

In some embodiments,

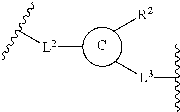

is selected from the following:

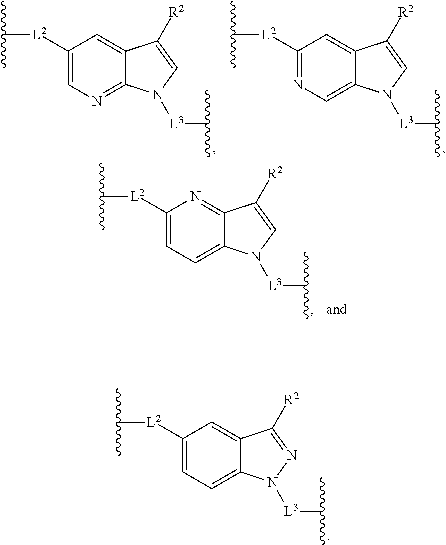

In some embodiments,

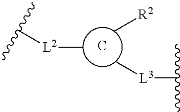

is selected from the following:

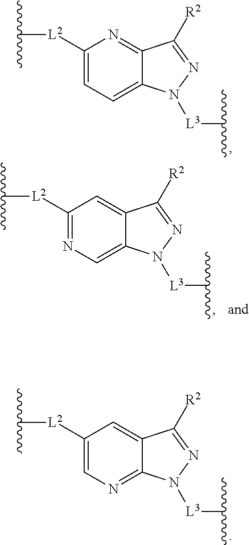

In some embodiments,

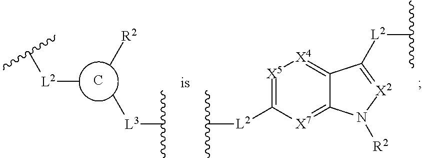

X² is —CH or N; X⁴ is —CH or N; X⁵ is —CH or N; and X⁷ is —CH or N, and wherein at least one X², X⁴, X⁵, or X⁷ is N.

In some embodiments,

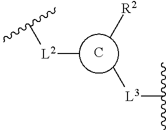

is selected from the following:

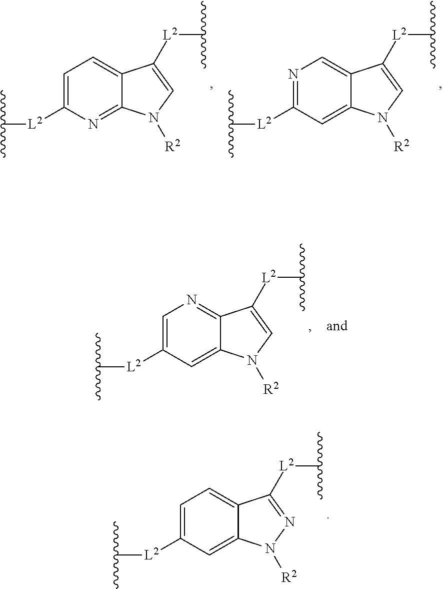

In some embodiments,

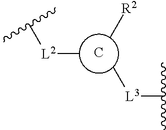

is selected from the following:

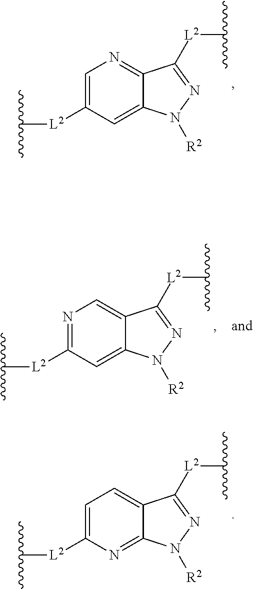

In some embodiments,

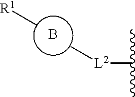

is selected from the following:

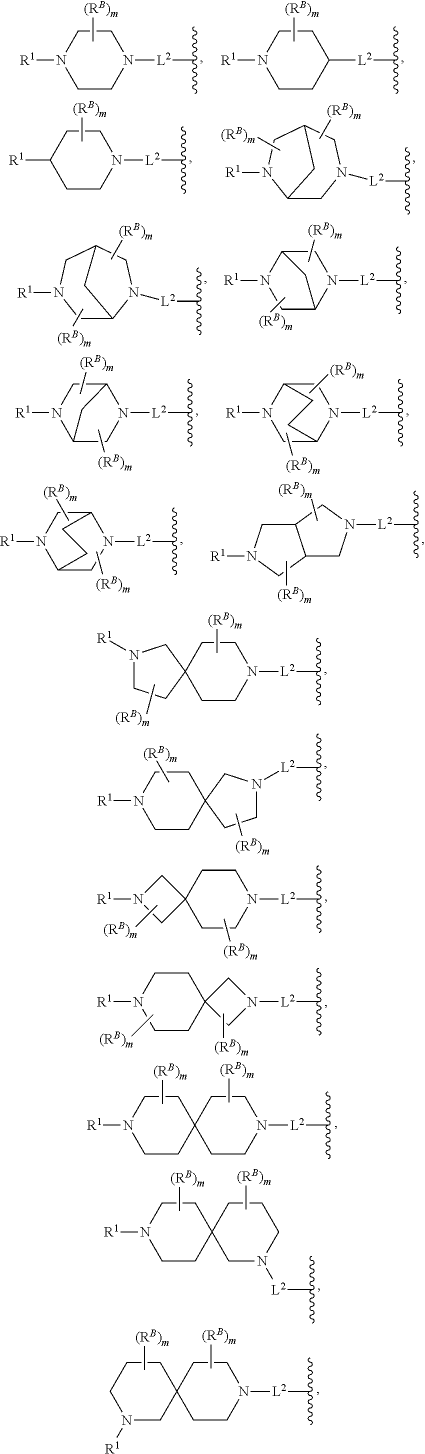

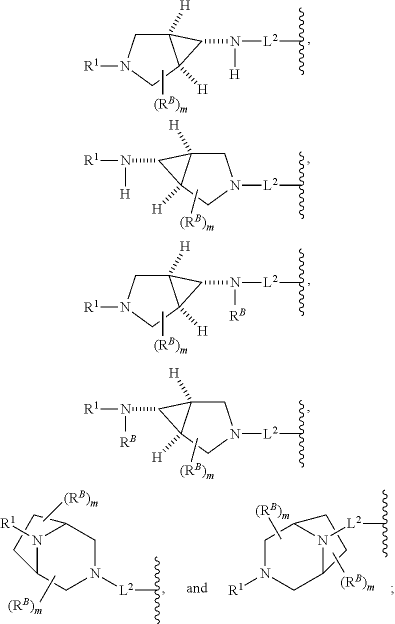

and each m is independently 0, 1, 2, 3, or 4.

In some embodiments,

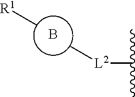

is selected from the following:

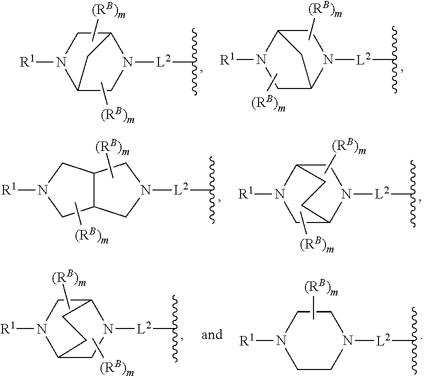

In some embodiments,

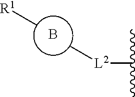

is selected from

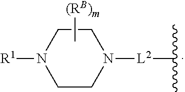

In some embodiments, the compound of Formula (IXa) has the following structure of Formula (IXb), or a pharmaceutically acceptable salt, or solvate thereof:

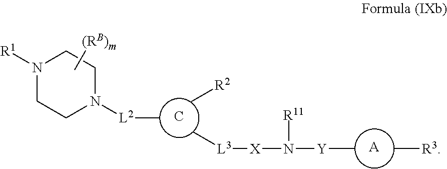

In some embodiments, the compound of Formula (IXa) has the following structure of Formula (IXc), or a pharmaceutically acceptable salt, or solvate thereof:

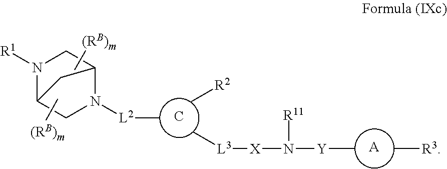

In some embodiments,

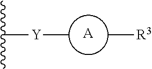

is selected from the following:

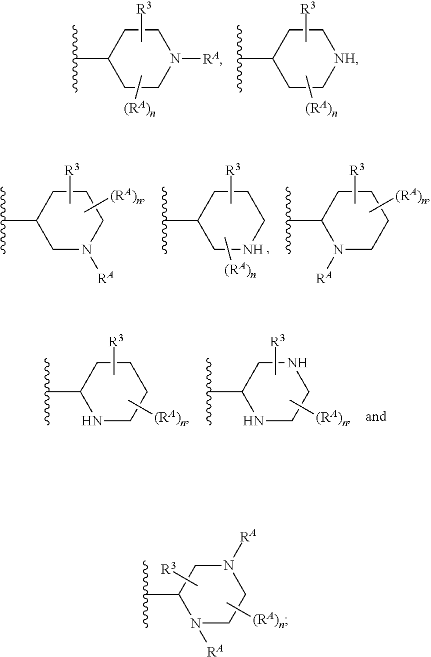

and each n is independently 0, 1, 2, 3, or 4.

In some embodiments,

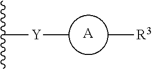

is selected from the following:

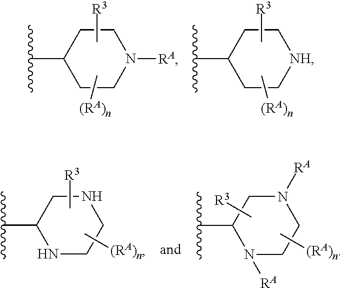

In some embodiments,

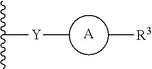

is selected from the following:

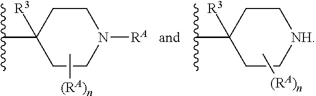

In some embodiments, L² is —CH₂—, C(═O), O, S, S(═O), S(═O)₂, or NR⁴. In some embodiments, L² is —CH₂—. In some embodiments, L³ is absent, —CH₂—, —CH₂—CH₂—, or —CH₂—CH₂—CH₂—. In some embodiments, L³ is —CH₂—CH₂—.

In some embodiments, L³ is:

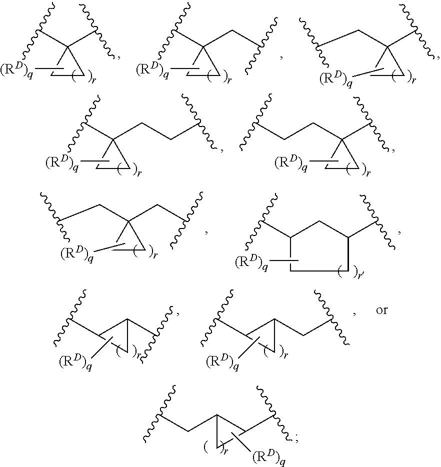

each q is independently 0, 1, 2, 3, or 4;

r is 1, 2, 3, 4, or 5; and

r′ is 1 or 2.

In some embodiments, X is —CH₂— or C(═O). In some embodiments, X is —CH₂—.

In some embodiments, X is:

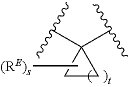

each s is independently 0, 1, 2, 3, or 4; and

t is 1, 2, 3, 4, or 5.

In some embodiments, X is

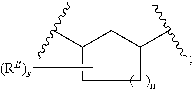

each s is independently 0, 1, 2, 3, or 4; and

u is 0, 1, or 2.

In some embodiments, L³-X is —CH₂—CH₂—CH₂—. In some embodiments, Y is —CH₂— or C(═O). In some embodiments, Y is C(═O). In some embodiments, R¹¹ is hydrogen. In some embodiments, R⁹ and R¹⁰ are each H. In some embodiments, R⁹ is H and R¹⁰ is —C₁-C₄alkylene-(optionally substituted phenyl) or —C₁-C₄alkylene-(optionally substituted heteroaryl). In some embodiments, R⁹ is H and R¹⁰ is —CH₂-(optionally substituted phenyl). In some embodiments, R⁹ is H and R¹⁰ is —CH₂-(optionally substituted heteroaryl). In some embodiments, R¹ is an hydrogen, an optionally substituted C₁-C₆alkyl, or an optionally substituted aryl. In some embodiments, R¹ is hydrogen. In some embodiments, R¹ is an unsubstituted C₁-C₆alkyl. In some embodiments, R¹ is a substituted C₁-C₆alkyl. In some embodiments, R¹ is an unsubstituted phenyl. In some embodiments, R¹ is a substituted phenyl. In some embodiments, R¹ is selected from:

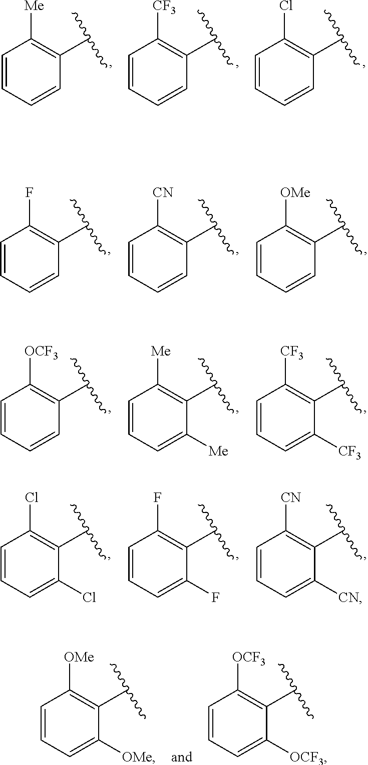

In some embodiments, R² is an unsubstituted phenyl. In some embodiments, R² is a substituted phenyl. In some embodiments, R² is selected from:

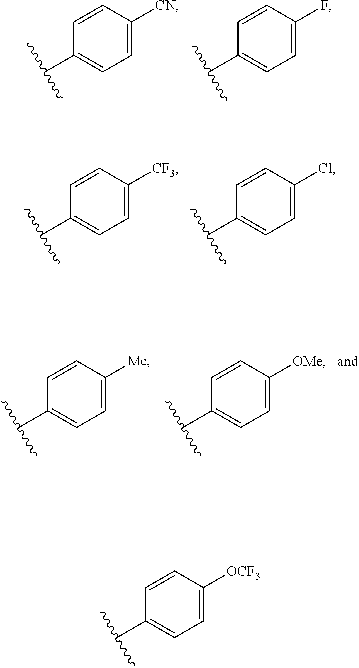

In some embodiments, the compound of Formula (IXa) has the following structure of Formula (IXd), or a pharmaceutically acceptable salt, or solvate thereof:

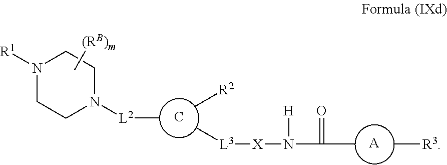

In some embodiments, the compound of Formula (IXa) has the following structure of Formula (IXe), or a pharmaceutically acceptable salt, or solvate thereof:

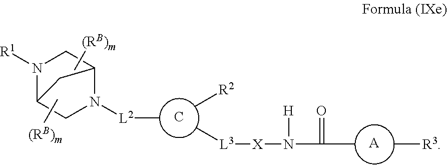

Also provided herein is a compound of Formula (Xa), or a pharmaceutically acceptable salt, or solvate thereof:

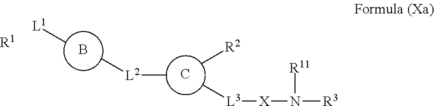

wherein,

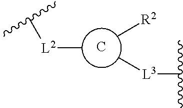

is a bicyclic heteroaryl or heterocycloalkyl that is selected from the following structures:

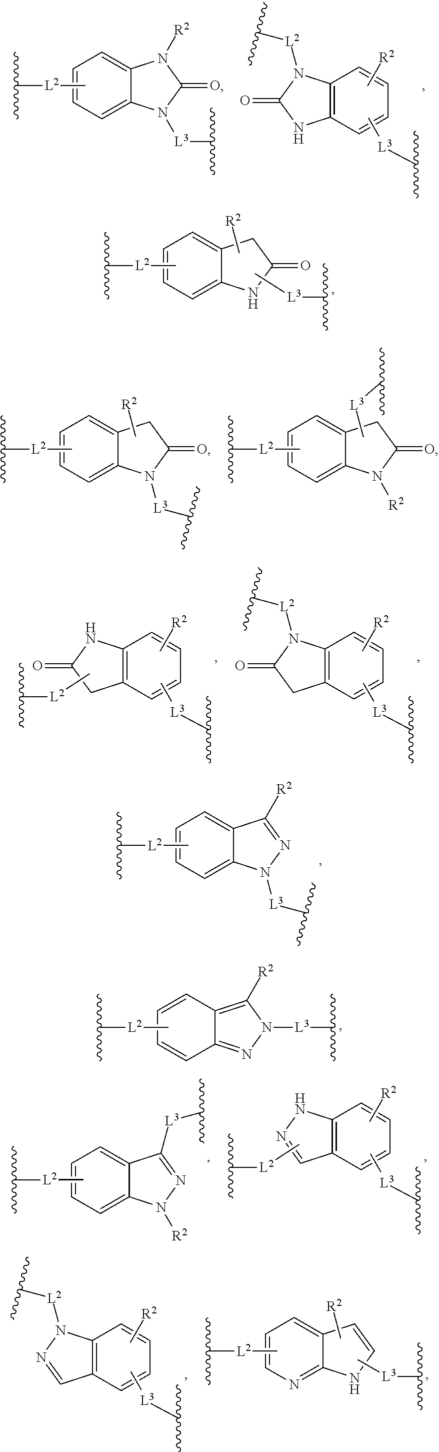

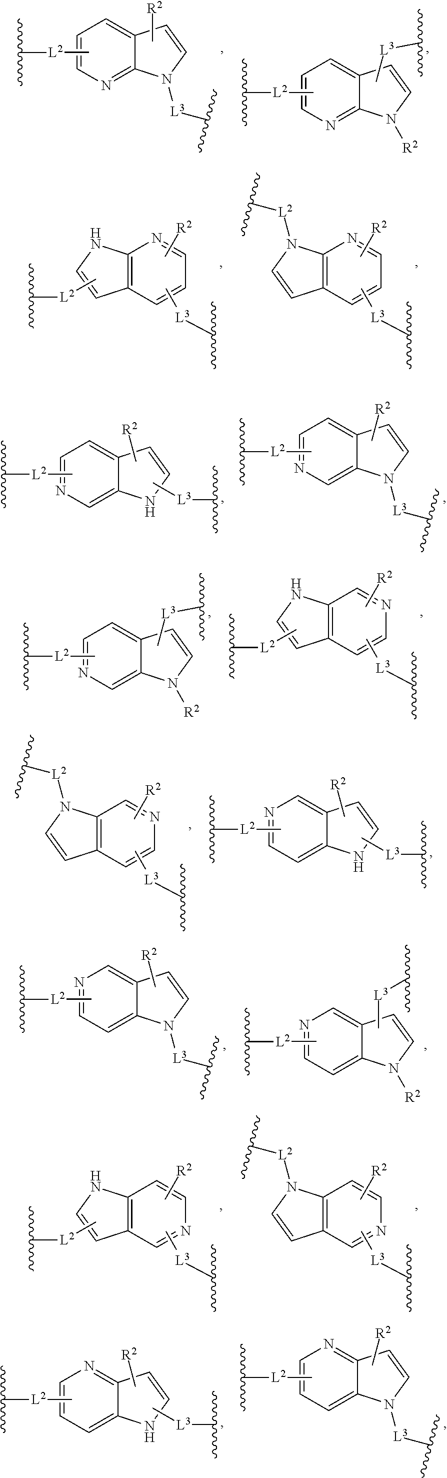

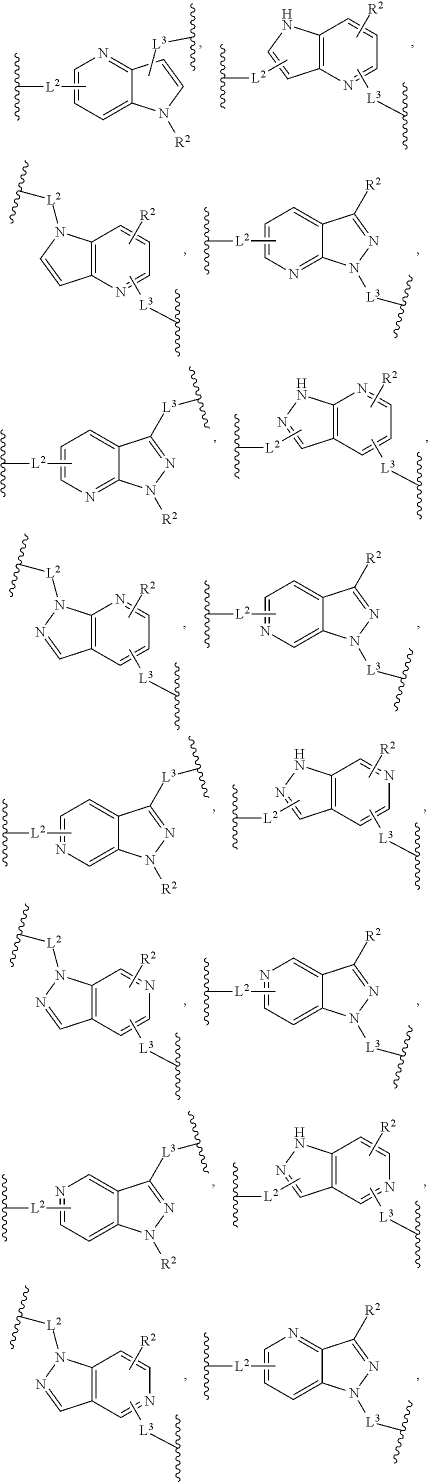

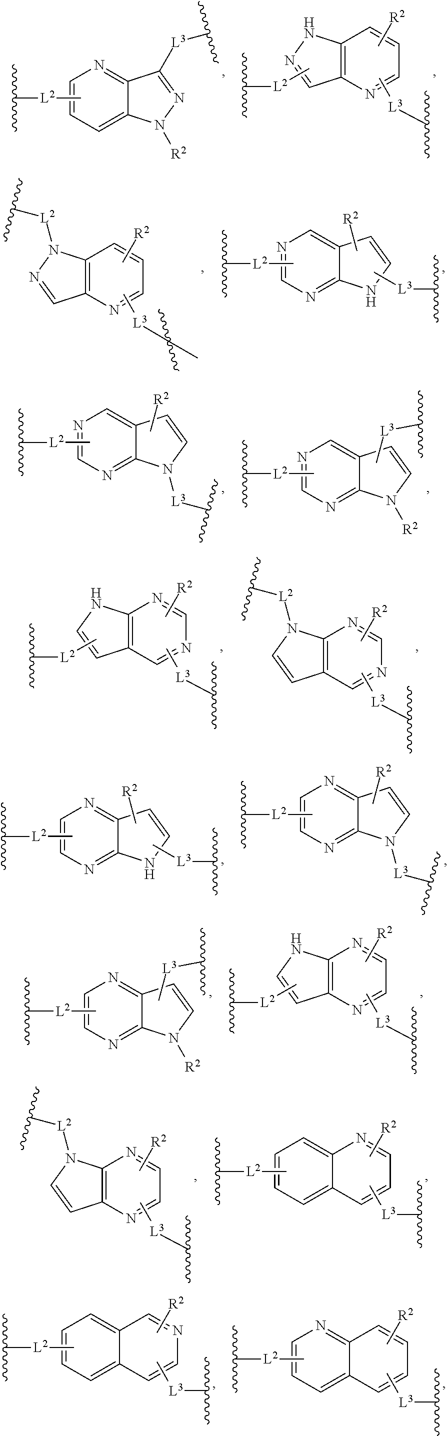

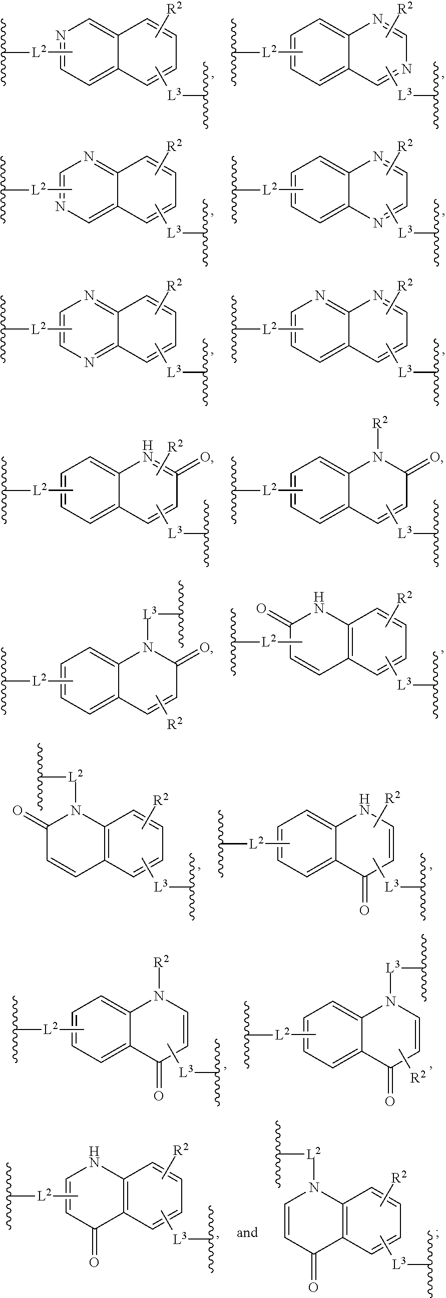

L¹ and L² are each independently absent, an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N;
wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted phenylene, an optionally substituted C₃-C₆cycloalkylene; an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)— or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
R³ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, —C₁-C₄alkylene-(optionally substituted heteroaryl), —CH₂C(═O)R¹⁵, —C(═O)R¹⁵, or —CO₂R¹⁶;
R⁴ is hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring,
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹⁵ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁶ is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl.

In some embodiments,

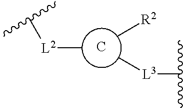

is selected from:

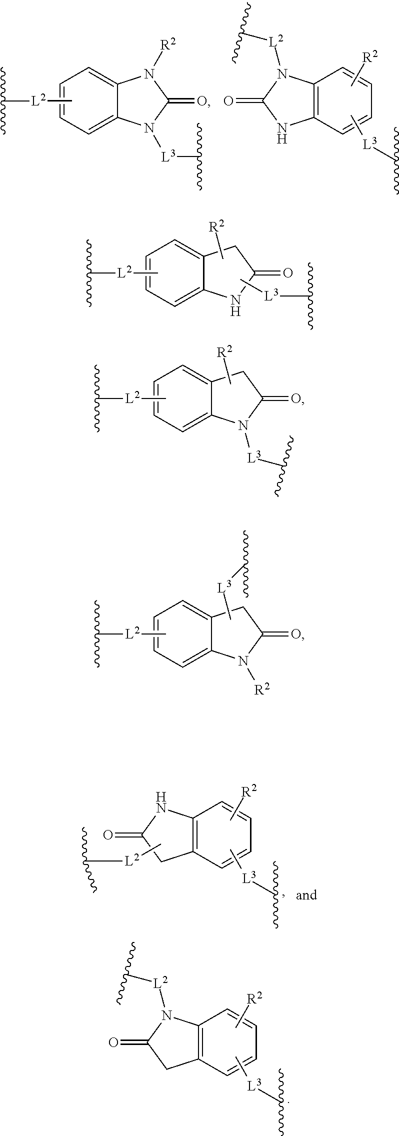

In some embodiments,

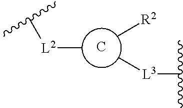

is selected from:

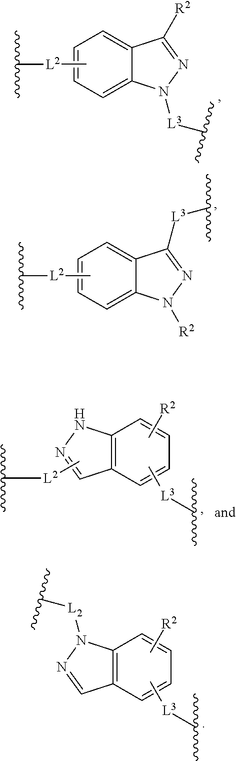

In some embodiments,

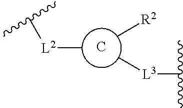

is selected from:

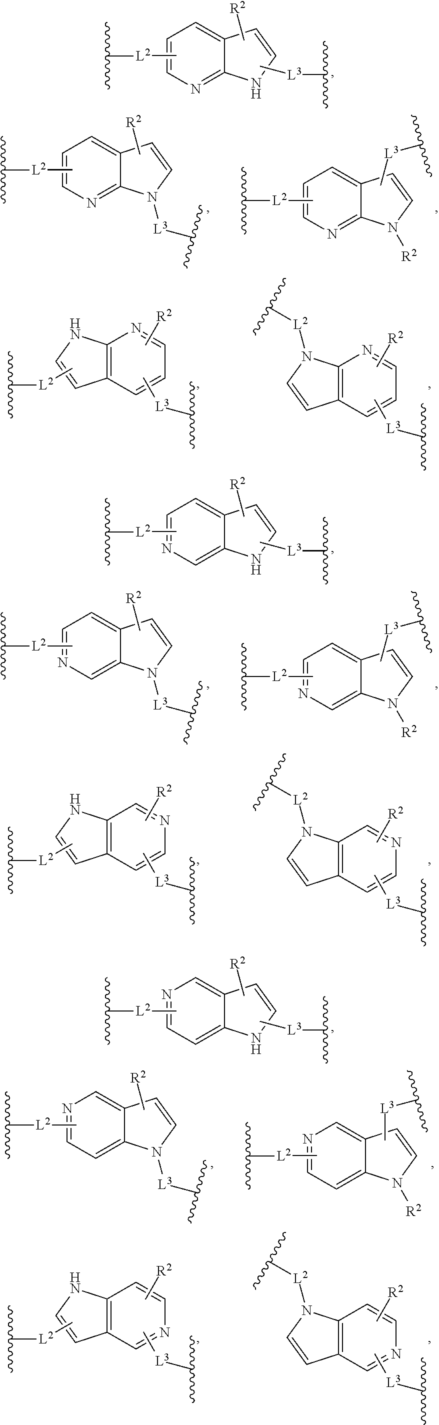

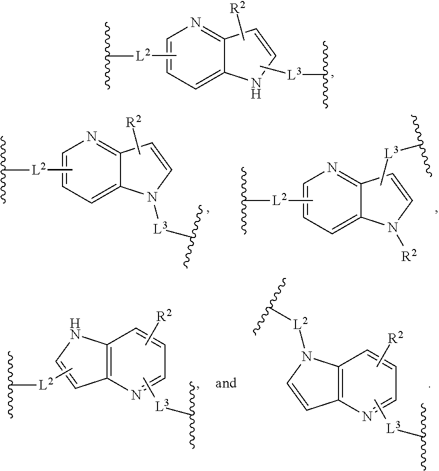

In some embodiments,

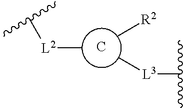

is selected from

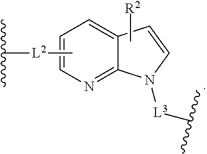

In some embodiments,

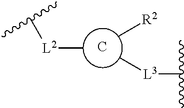

is selected from:

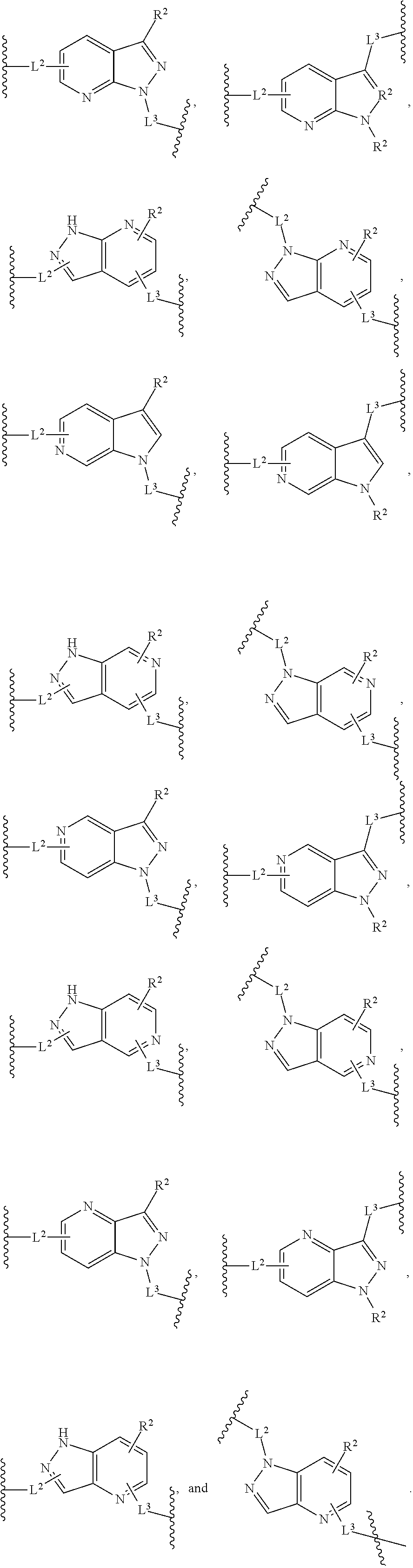

In some embodiments,

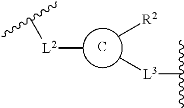

is selected from:

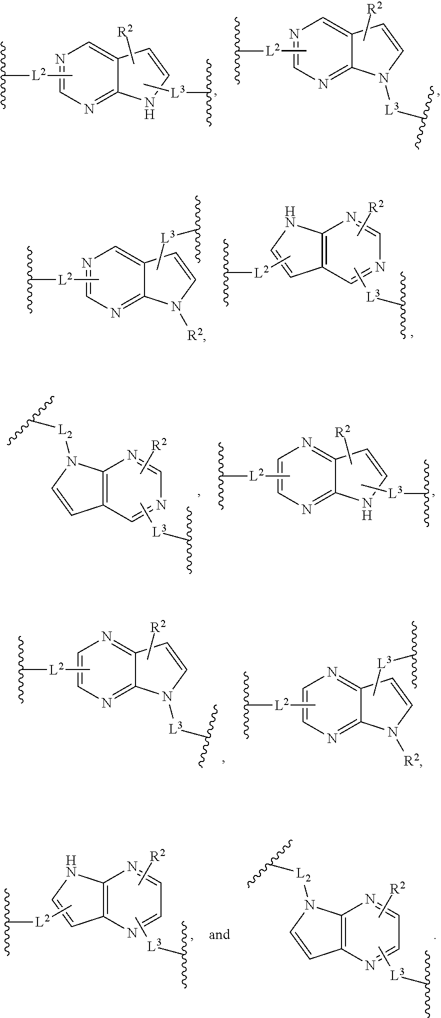

In some embodiments,

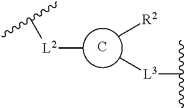

is selected from:

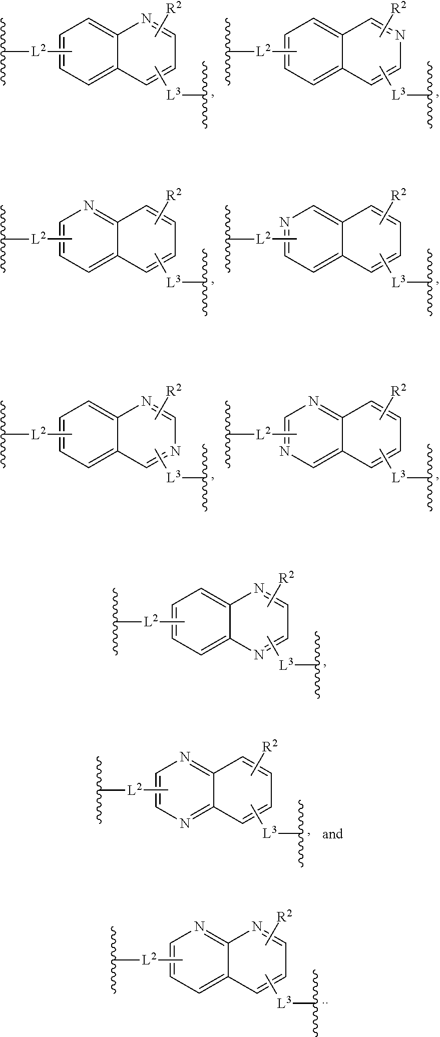

In some embodiments,

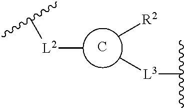

is selected from:

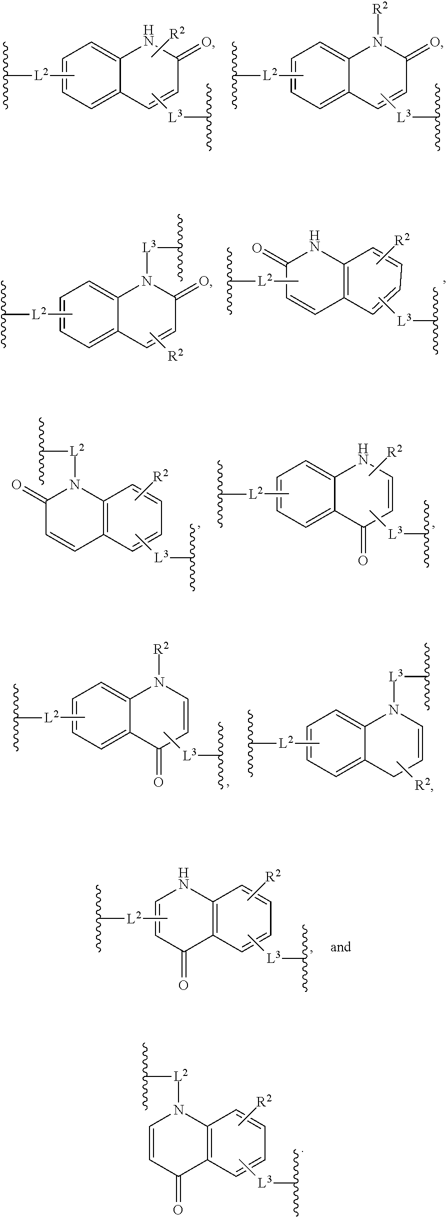

In some embodiments,

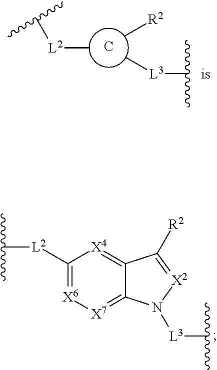

X² is —CH or N; X⁴ is —CH or N; X⁶ is —CH or N; and X⁷ is —CH or N, and wherein at least one X², X⁴, X⁶, or X⁷ is N.

In some embodiments,

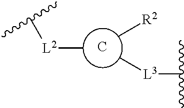

is selected from the following:

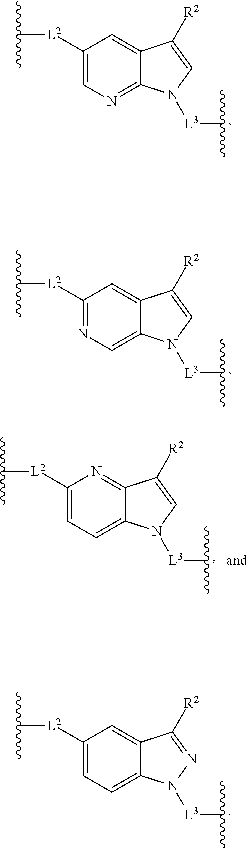

In some embodiments,

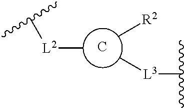

is selected from the following:

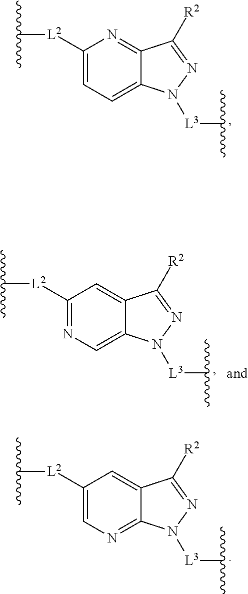

In some embodiments,

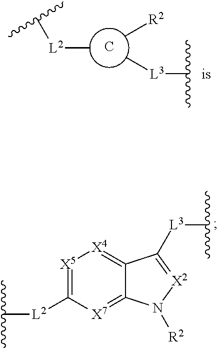

X² is —CH or N; X⁴ is —CH or N; X⁵ is —CH or N; and X⁷ is —CH or N, and wherein at least one X², X⁴, X⁵, or X⁷ is N.

In some embodiments,

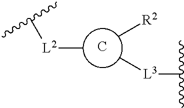

is selected from the following:

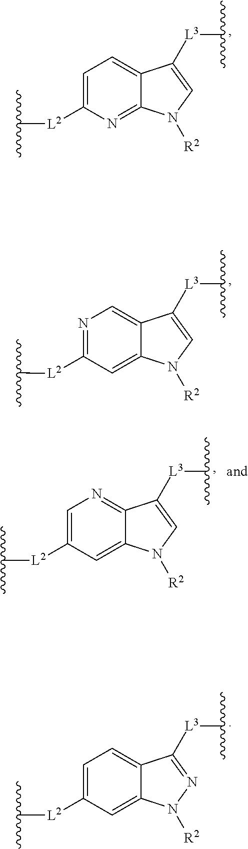

In some embodiments,

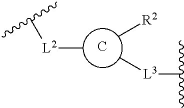

is selected from the following:

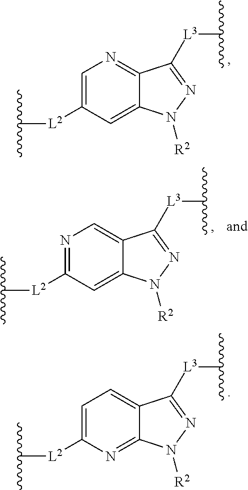

In some embodiments,

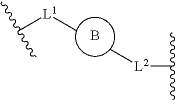

is selected from the following:

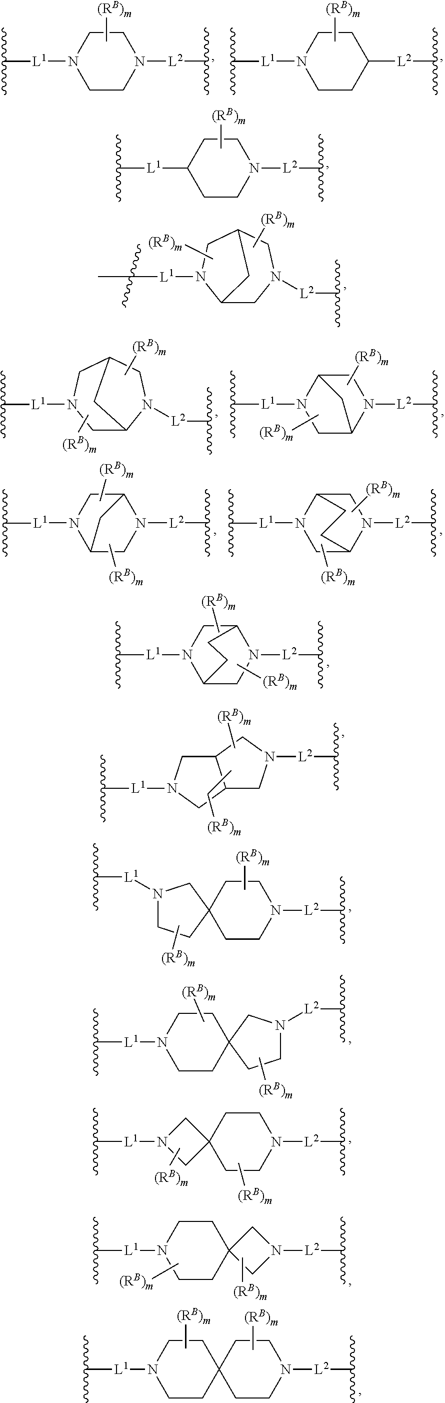

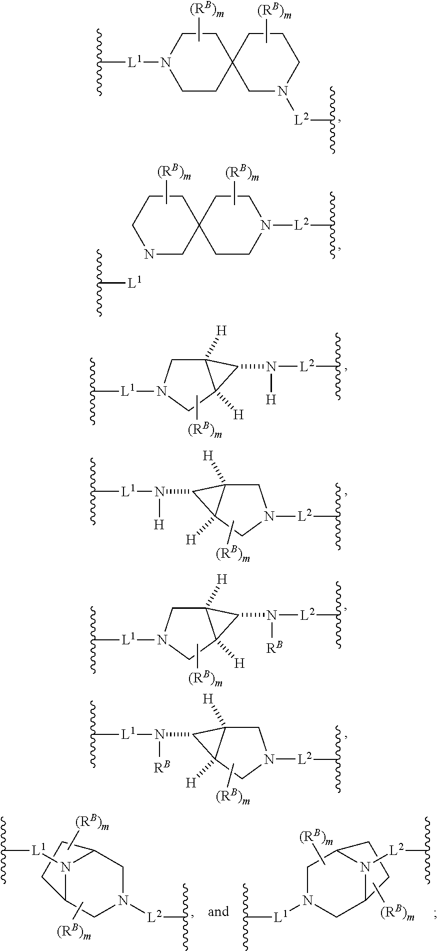

and each m is independently 0, 1, 2, 3, or 4.

In some embodiments,

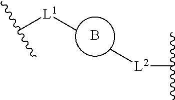

is selected from the following:

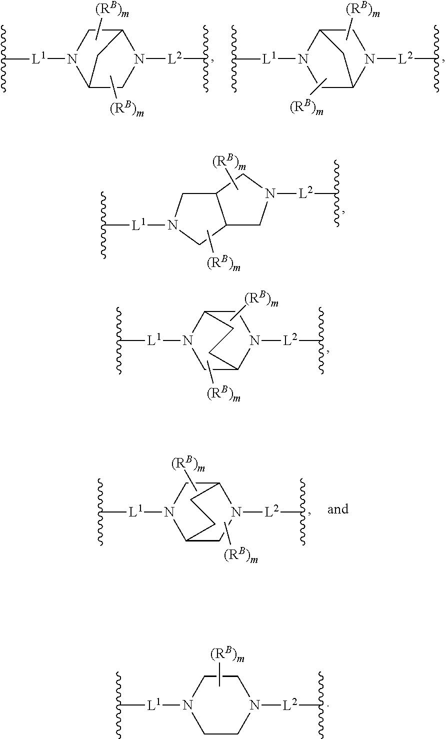

In some embodiments,

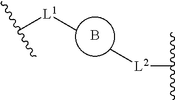

is selected from

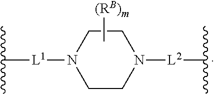

In some embodiments, the compound of Formula (Xa) has the following structure of Formula (Xb), or a pharmaceutically acceptable salt, or solvate thereof:

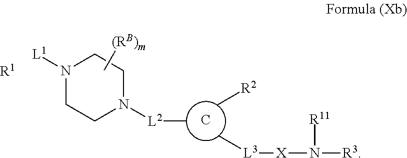

In some embodiments, the compound of Formula (Xa) has the following structure of Formula (Xc), or a pharmaceutically acceptable salt, or solvate thereof:

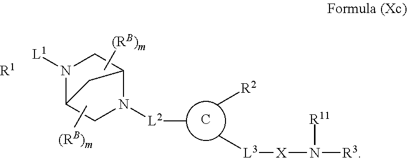

In some embodiments, L¹ is —CH₂—, C(═O), O, S, S(═O), S(═O)₂, or NR⁴. In some embodiments, L¹ is —CH₂—. In some embodiments, L² is —CH₂—, C(═O), O, S, S(═O), S(═O)₂, or NR⁴. In some embodiments, L² is —CH₂—. In some embodiments, L³ is absent, —CH₂—, —CH₂—CH₂—, or —CH₂—CH₂—CH₂—. In some embodiments, L³ is —CH₂—CH₂—.

In some embodiments, L³ is:

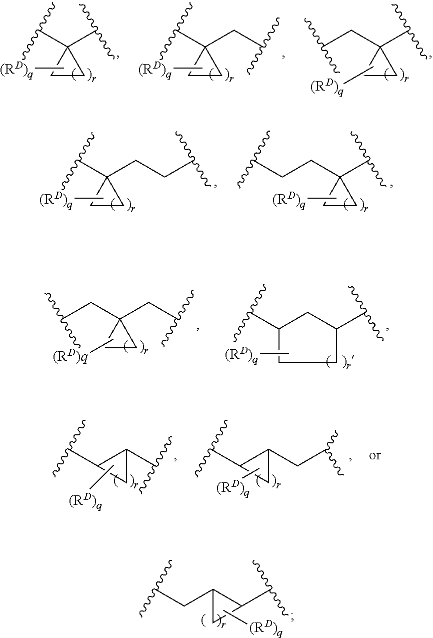

each q is independently 0, 1, 2, 3, or 4;

r is 1, 2, 3, 4, or 5; and

r′ is 1 or 2.

In some embodiments, X is —CH₂— or C(═O). In some embodiments, X is —CH₂—.

In some embodiments, X is:

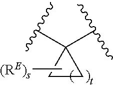

each s is independently 0, 1, 2, 3, or 4; and

t is 1, 2, 3, 4, or 5.

In some embodiments, X is:

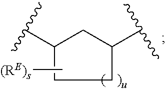

each s is independently 0, 1, 2, 3, or 4; and

u is 0, 1, or 2.

In some embodiments, L³-X is —CH₂—CH₂—CH₂—. In some embodiments, R¹¹ and R³ are each independently hydrogen, optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹¹ and R³ are each hydrogen. In some embodiments, R¹¹ and R³ are each optionally substituted C₁-C₆alkyl. In some embodiments, R¹¹ and R³ are each optionally substituted C₁-C₆heteroalkyl. In some embodiments, R¹⁵ and R¹⁶ are each independently optionally substituted C₁-C₆alkyl or optionally substituted C₁-C₆heteroalkyl. In some embodiments, R¹¹ is hydrogen and R³ is optionally substituted C₁-C₆alkyl, optionally substituted C₁-C₆heteroalkyl, —CH₂C(═O)R¹⁵, —C(═O)R¹⁵, or —CO₂R¹⁶. In some embodiments, R¹ is an unsubstituted phenyl. In some embodiments, R¹ is a substituted phenyl.

In some embodiments, R¹ is selected from:

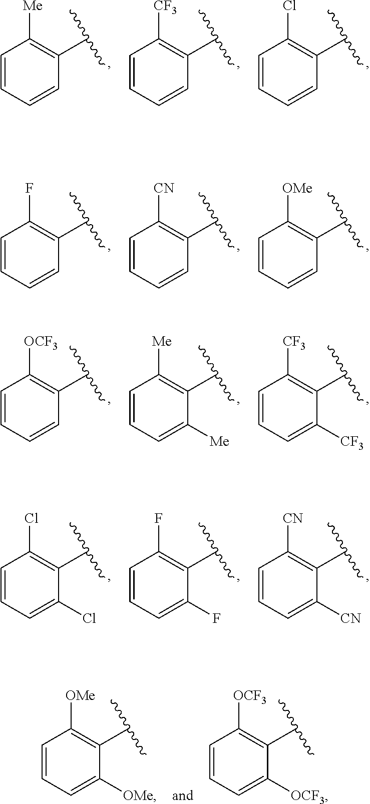

In some embodiments, R² is an unsubstituted phenyl. In some embodiments, R² is a substituted phenyl. In some embodiments, R² is selected from:

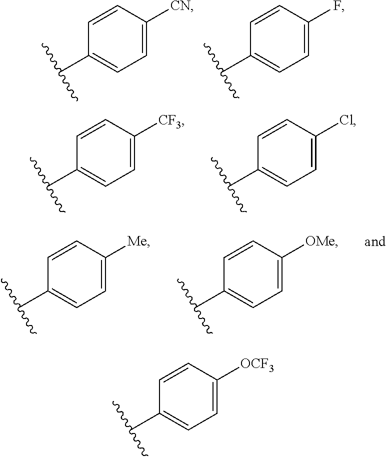

In some embodiments, the compound of Formula (Xa) has the following structure of Formula (Xd), or a pharmaceutically acceptable salt, or solvate thereof:

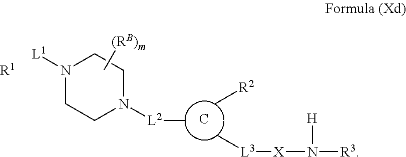

In some embodiments, the compound of Formula (Xa) has the following structure of Formula (Xe), or a pharmaceutically acceptable salt, or solvate thereof:

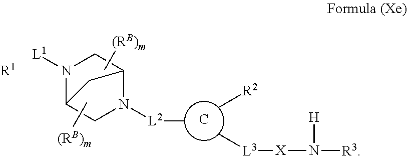

In some embodiments, the compound disclosed herein is a compound from the following table/

TABLE A9

| Com- | | |
|---|---|---|
| pound | | |
| Ref. | Compound | Chemical Name |
| C174 | 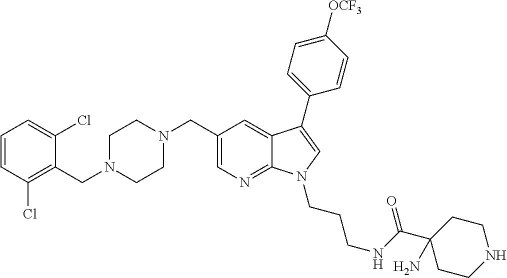 [SMILES: NC1(C(=O)NCCCn2cc(-c3ccc(OC(F)(F)F)cc3)c3cc(CN4CCN(Cc5c(Cl)cccc5Cl)CC4)cnc32)CCNCC1] | 4-amino-N-(3-(5-((4-(2,6- dichlorobenzyl)piperazin-1- yl)methyl)-3-(4- (trifluoromethoxy)phenyl)-1H- pyrrolo[2,3-b]pyridin-1- yl)propyl)piperidine-4- carboxamide |
| C175 | 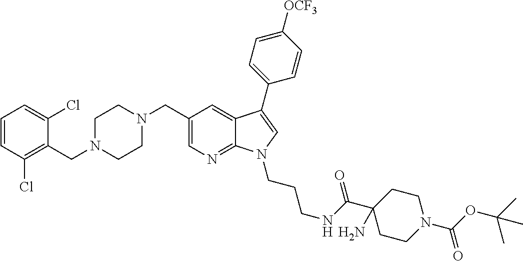 [SMILES: CC(C)(C)OC(=O)N1CCC(N)(C(=O)NCCCn2cc(-c3ccc(OC(F)(F)F)cc3)c3cc(CN4CCN(Cc5c(Cl)cccc5Cl)CC4)cnc32)CC1] | tert-butyl 4-amino-4-((3-(5- ((4-(2,6- dichlorobenzyl)piperazin-1- yl)methyl)-3-(4- (trifluoromethoxy)phenyl)- 1H-pyrrolo[2,3-b]pyridin-1- yl)propyl)carbamoyl) piperidine-1-carboxylate |
| C176 | 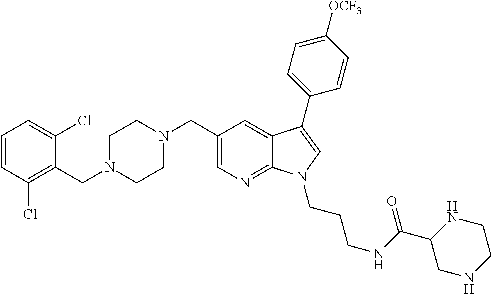 [SMILES: O=C(NCCCn1cc(-c2ccc(OC(F)(F)F)cc2)c2cc(CN3CCN(Cc4c(Cl)cccc4Cl)CC3)cnc21)C1CNCCN1] | N-(3-(5-((4-(2,6- dichlorobenzyl)piperazin-1- yl)methyl)-3-(4- (trifluoromethoxy)phenyl)- 1H-pyrrolo[2,3-b]pyridin-1- yl)propyl)piperazine-2- carboxamide |
| C177 | 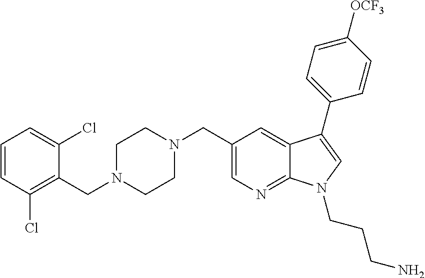 [SMILES: NCCCn1cc(-c2ccc(OC(F)(F)F)cc2)c2cc(CN3CCN(Cc4c(Cl)cccc4Cl)CC3)cnc21] | 3-(5-((4-(2,6- dichlorobenzyl)piperazin-1- yl)methyl)-3-(4- (trifluoromethoxy)phenyl)- 1H-pyrrolo[2,3-b]pyridin-1- yl)propan-1-amine |
| C178 | 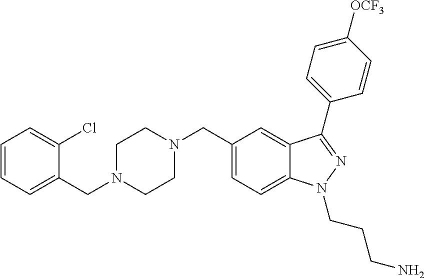 [SMILES: NCCCn1nc(-c2ccc(OC(F)(F)F)cc2)c2cc(CN3CCN(Cc4ccccc4Cl)CC3)ccc21] | 3-(5-((4-(2- chlorobenzyl)piperazin-1- yl)methyl)-3-(4- (trifluoromethoxy)phenyl)- 1H-indazol-1-yl)propan- 1-amine |
| C182 | 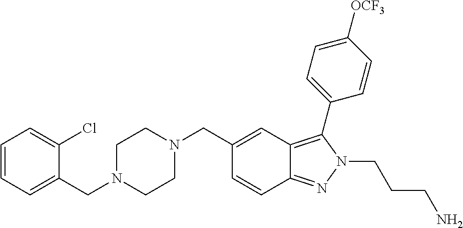 [SMILES: NCCCn1nc2ccc(CN3CCN(Cc4ccccc4Cl)CC3)cc2c1-c1ccc(OC(F)(F)F)cc1] | 3-(5-((4-(2- chlorobenzyl)piperazin-1- yl)methyl)-3-(4- (trifluoromethoxy)phenyl)- 2H-indazol-2-yl) propan-1-amine |
| C200 | 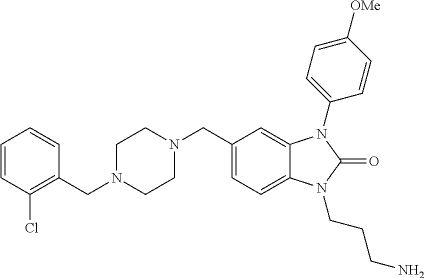 [SMILES: COc1ccc(-n2c(=O)n(CCCN)c3ccc(CN4CCN(Cc5ccccc5Cl)CC4)cc32)cc1] | 1-(3-aminopropyl)-5-((4-(2- chlorobenzyl)piperazin-1- yl)methyl)-3-(4- methoxyphenyl)-1,3-dihydro- 2H-benzo[d]imidazol-2-one |
| C201 | 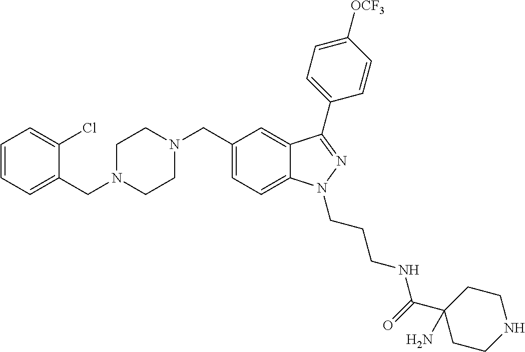 [SMILES: NC1(C(=O)NCCCn2nc(-c3ccc(OC(F)(F)F)cc3)c3cc(CN4CCN(Cc5ccccc5Cl)CC4)ccc32)CCNCC1] | 4-amino-N-(3-(5-((4-(2- chlorobenzyl)piperazin-1- yl)methyl)-3-(4- (trifluoromethoxy)phenyl)- 1H-indazol-1-yl)propyl) piperidine-4-carboxamide |
| C207 | 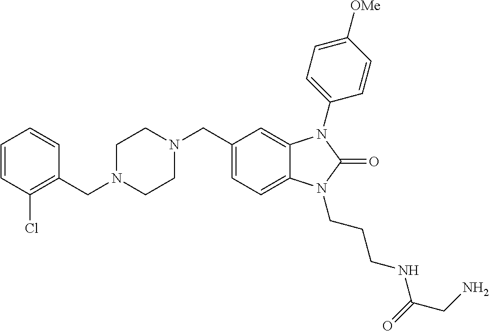 [SMILES: COc1ccc(-n2c(=O)n(CCCNC(=O)CN)c3ccc(CN4CCN(Cc5ccccc5Cl)CC4)cc32)cc1] | 2-amino-N-(3-(5-((4-(2- chlorobenzyl)piperazin-1- yl)methyl)-3-(4- methoxyphenyl)-2-oxo-2,3- dihydro-1H-benzo[d] imidazol-1-yl)propyl) acetamide |

Any combination of the groups described above for the various variables is contemplated herein. Throughout the specification, groups and substituents thereof are chosen by one skilled in the field to provide stable moieties and compounds.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the metabolites, pharmaceutically acceptable salts, esters, prodrugs, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Prodrugs

In some embodiments, the compounds described herein exist in prodrug form. The invention provides for methods of treating diseases by administering such prodrugs. The invention further provides for methods of treating diseases by administering such prodrugs as pharmaceutical compositions.

In some embodiments, prodrugs include compounds wherein an amino acid residue, or a polypeptide chain of two or more (e. g., two, three or four) amino acid residues is covalently joined through an amide or ester bond to a free amino, hydroxy or carboxylic acid group of compounds of the present invention. The amino acid residues include but are not limited to the 20 naturally occurring amino acids and also includes 4-hydroxyproline, hydroxylysine, demosine, isodemosine, 3-methylhistidine, norvaline, beta-alanine, gamma-aminobutyric acid, cirtulline, homocysteine, homoserine, omithine and methionine sulfone. In other embodiments, prodrugs include compounds wherein a nucleic acid residue, or an oligonucleotide of two or more (e. g., two, three or four) nucleic acid residues is covalently joined to a compound of the present invention.

Pharmaceutically acceptable prodrugs of the compounds described herein also include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. In some embodiments, compounds having free amino, amido, hydroxy or carboxylic groups are converted into prodrugs. For instance, free carboxyl groups are derivatized as amides or alkyl esters. In certain instances, all of these prodrug moieties incorporate groups including but not limited to ether, amine and carboxylic acid functionalities.

Hydroxy prodrugs include esters, such as though not limited to, acyloxyalkyl (e.g. acyloxymethyl, acyloxyethyl) esters, alkoxycarbonyloxyalkyl esters, alkyl esters, aryl esters, phosphate esters, sulfonate esters, sulfate esters and disulfide containing esters; ethers, amides, carbamates, hemisuccinates, dimethylaminoacetates and phosphoryloxymethyloxycarbonyls, as outlined in Advanced Drug Delivery Reviews 1996, 19, 115.

Amine derived prodrugs include, but are not limited to the following groups and combinations of groups:

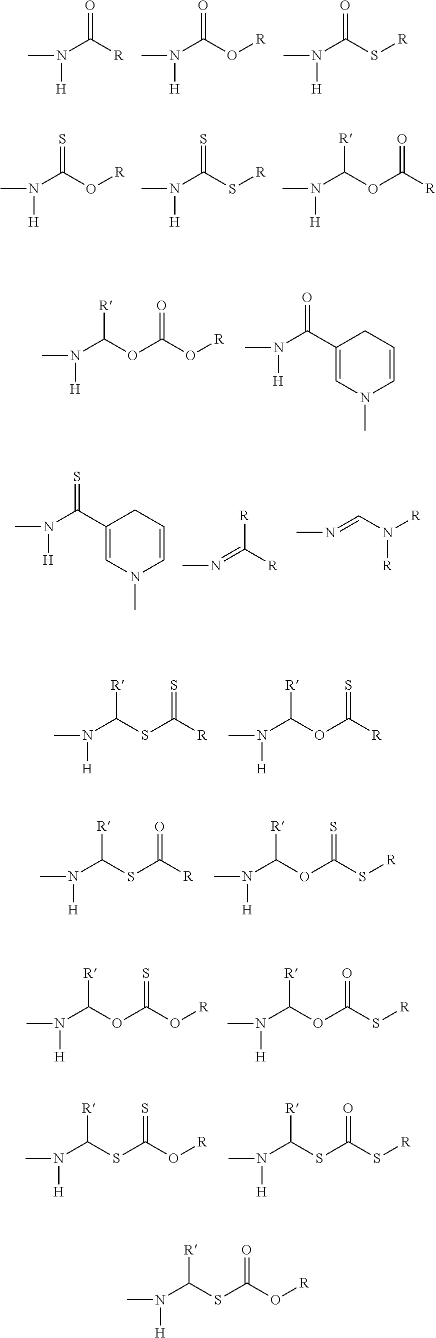

as well as sulfonamides and phosphonamides.

In certain instances, sites on any aromatic ring portions are susceptible to various metabolic reactions, therefore incorporation of appropriate substituents on the aromatic ring structures, reduce, minimize or eliminate this metabolic pathway in some embodiments.

### Metabolites

In some embodiments, the compounds described herein are susceptible to various metabolic reactions. Therefore, in some embodiments, incorporation of appropriate substituents into the structure will reduce, minimize, or eliminate a metabolic pathway. In specific embodiments, the appropriate substituent to decrease or eliminate the susceptibility of an aromatic ring to metabolic reactions is, by way of example only, a halogen, or an alkyl group.

In additional or further embodiments, the compounds described herein are metabolized upon administration to an organism in need to produce a metabolite that is then used to produce a desired effect, including a desired therapeutic effect.

### Synthesis of Compounds

The compounds of described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

The compounds described herein are prepared by the general synthetic route described below in Schemes 1-2.

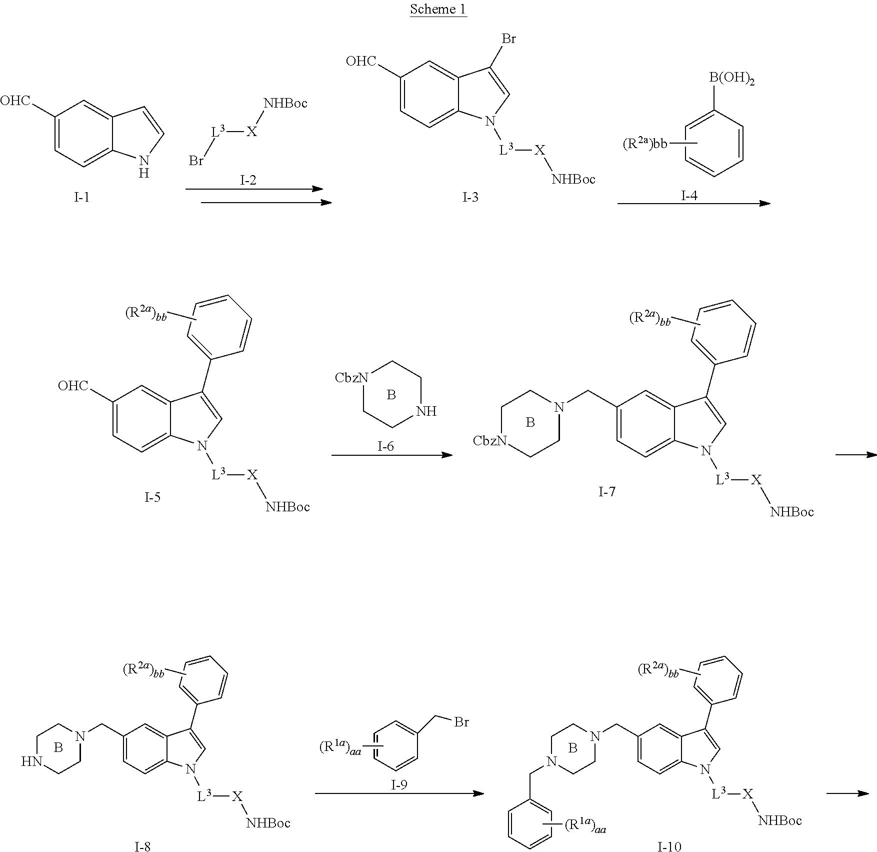

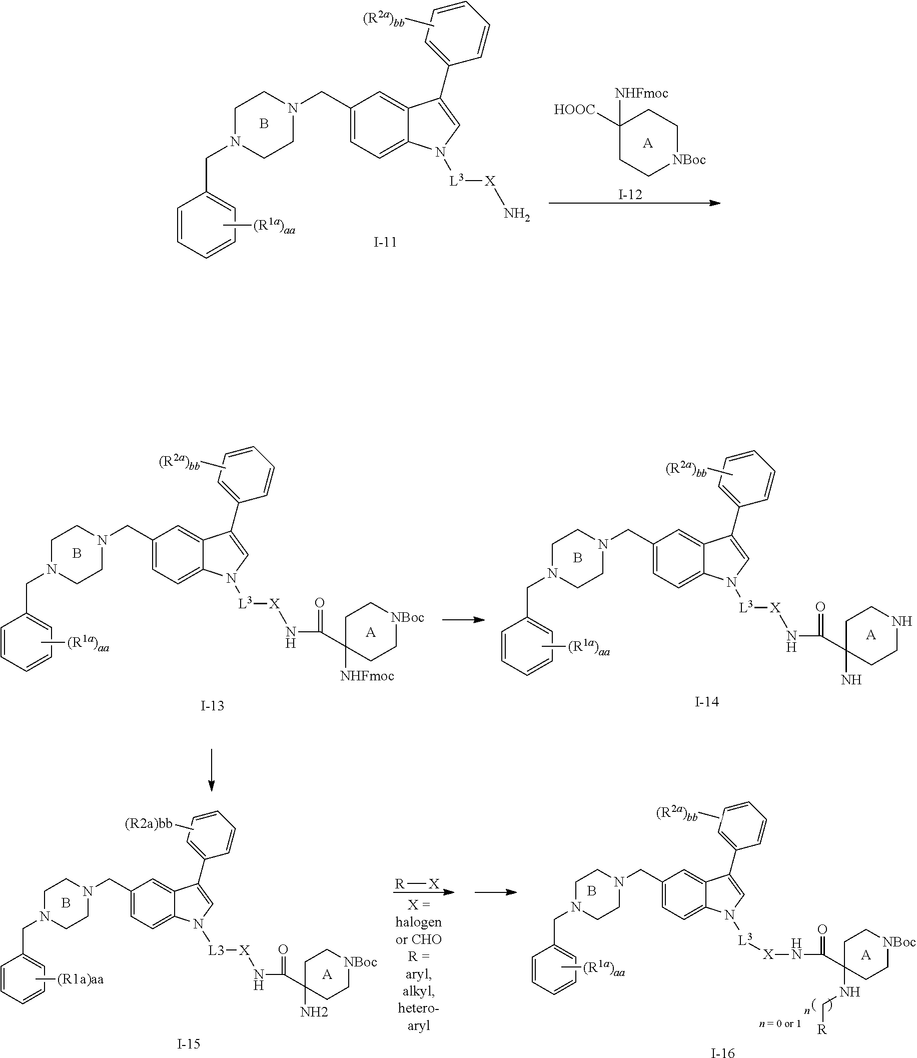

In some embodiments, the compounds as shown in Scheme 1 are compounds of Formula (Ia). In some embodiments L³-X is —CH₂—CH₂—CH₂— and Ring B is an optionally substituted monocyclic or bicyclic heterocycloalkyl ring containing at least one N with the proviso that Ring B is not

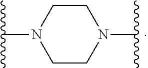 [SMILES: *N1CCN(*)CC1]

In some embodiments, Ring A is an optionally substituted heterocycloalkyl ring containing at least one N.

In some embodiments, the compounds as shown in Scheme 1 are compounds of Formula (IIa). In some embodiments, L³-X is a substituted C₃alkylene and Ring B is piperazine. In some embodiments, Ring A is an optionally substituted heterocycloalkyl ring containing at least one N.

In Scheme 1, each R^{1a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; and each aa is 0, 1, or 2. In some embodiments, each R^{1a} is H. In some embodiments, each R^{1a} is halogen. In some embodiments, aa is 0. In some embodiments, aa is 1. In some embodiments, aa is 2. In some embodiments, each R^{1a} is Cl and aa is 2. In some embodiments, R^{1a} is Cl and aa is 1.

In Scheme 1, each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2. In some embodiments, R^{2a} is halogen. In some embodiments, R^{2a} is —OCF₃. In some embodiments, bb is 0. In some embodiments bb is 1. In some embodiments, R^{2a} is halogen and bb is 1. In some embodiments, R^{2a} is —OCF₃ and bb is 1.

In some embodiments, the compounds of I-14 are prepared as shown in Scheme 1. In some embodiments, a suitable indole aldehyde, such as compound I-1, is alkylated on the indole nitrogen with a suitable alkylating agent, such as compound I-2, after removal of the indole NH with a suitable metal reagent, such as sodium hydride. In some embodiments, the resultant compound is then brominated with a suitable brominating reagent at the 3 position to provide compound I-3. In some embodiments, compound I-3 is then subjected under standard Pd coupling with an appropriate suitable arylboronic acid, such as compound I-4, to provide compound I-5. In some embodiments, compound I-5 is reacted with a singly blocked diamino compound, such as compound I-6, under suitable reductive amination conditions to afford compound I-7. In some instances, the blocking group of compound I-7 is selectively removed under suitable conditions to provide compound I-8. In some embodiments, the free NH of compound I-8 is either alkylated with a suitable benzyl bromide, such as compound I-9, or reductively aminated with an aryl aldehyde to provide a benzylic type compound I-10. In some embodiments, the Boc group of compound 10 is removed under suitable conditions to provide compound I-11. In some embodiments, resultant free amine of compound I-11 is coupled to an appropriate amino acid, such as compound I-12, under suitable reaction conditions to provide compound I-13. In some instances, the amino acid blocking groups are then removed under appropriate conditions to afford compound I-14.

Alternatively, the compound of I-13 is subjected under appropriate reaction conditions to provide compound I-15. In some instances, further reaction of compound I-15 under reductive amination or alkylation conditions with the appropriate reagent R—X followed by treatment under appropriate conditions to cleave the Boc group, such as acidic conditions, affords compound I-16.

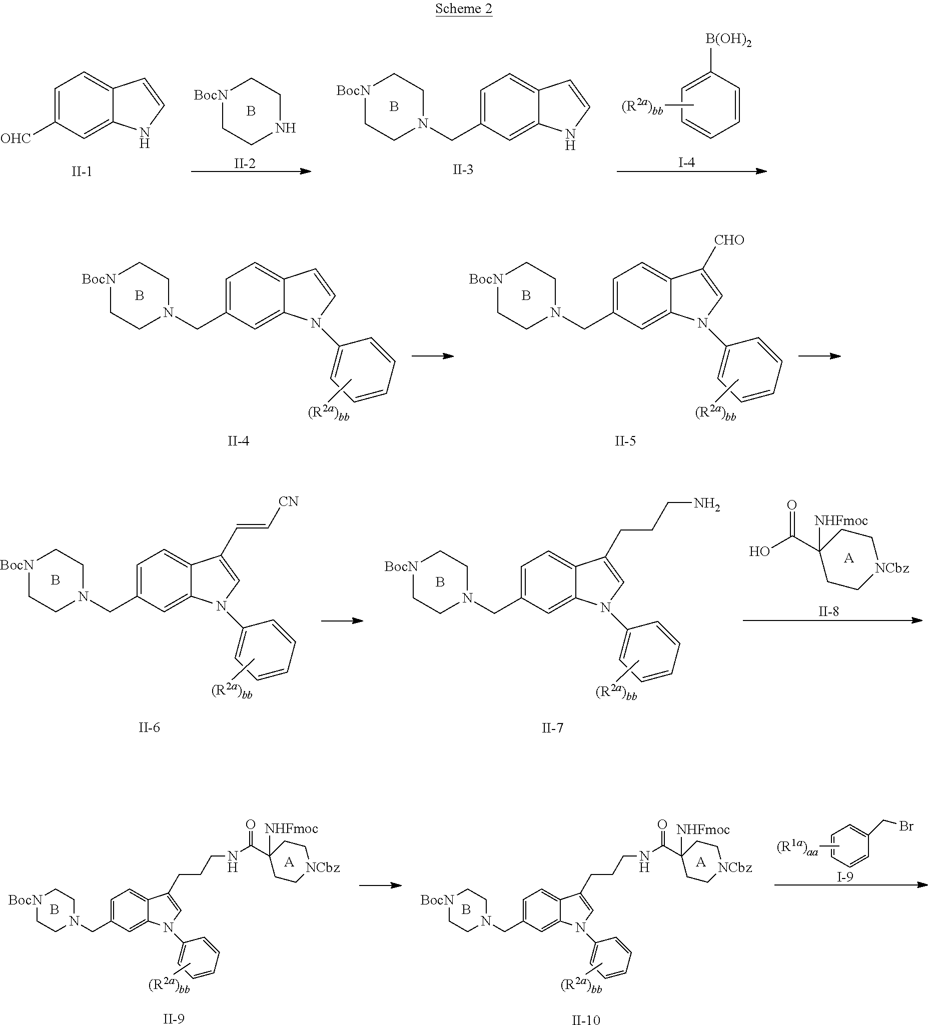

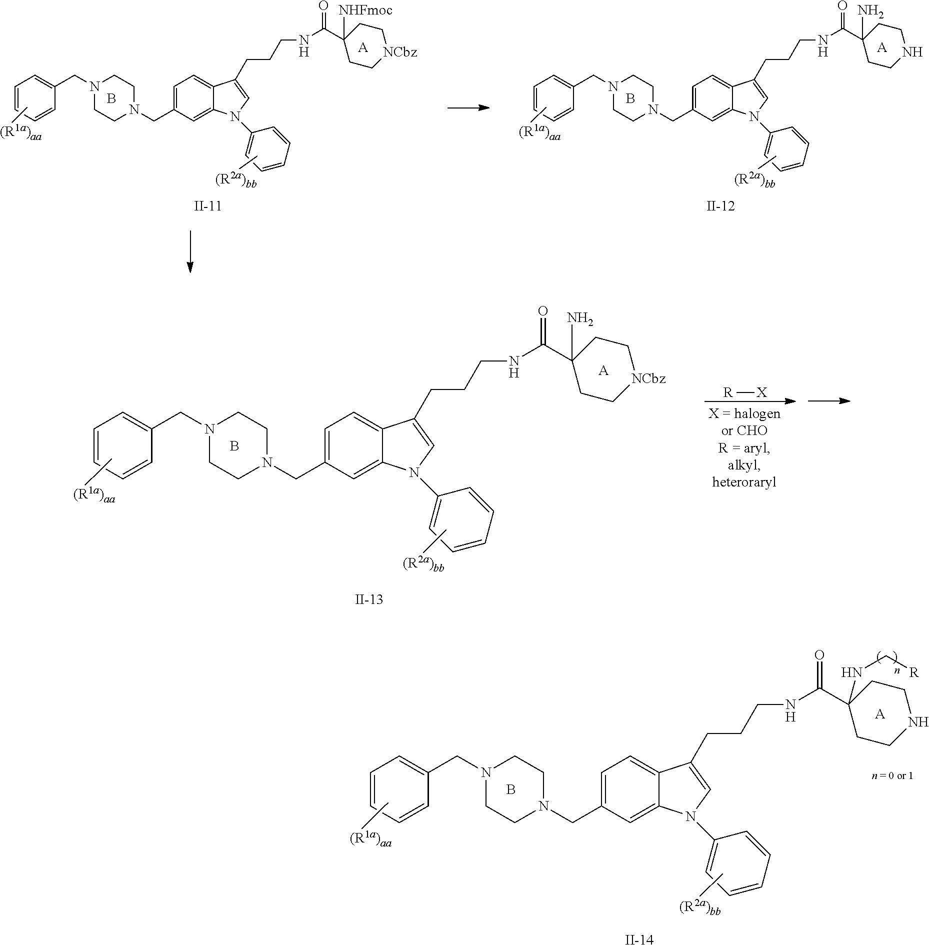

In some embodiments, the compounds as shown in Scheme 2 are compounds of Formula (Ia). In some embodiments Ring B is as an optionally substituted monocyclic or bicyclic heterocycloalkyl ring containing at least one N with the proviso that Ring B is not

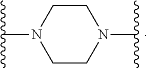 [SMILES: *N1CCN(*)CC1]

In some embodiments, Ring A is an optionally substituted heterocycloalkyl ring containing at least one N.

In Scheme 2, each R^{1a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; and each aa is 0, 1, or 2. In some embodiments, each R^{1a} is H. In some embodiments, each R^{1a} is halogen. In some embodiments, aa is 0. In some embodiments, aa is 1. In some embodiments, aa is 2. In some embodiments, each R^{1a} is Cl and aa is 2. In some embodiments, R^{1a} is Cl and aa is 1.

In Scheme 2, each R^{2a} is independently H, CN, CF₃, halogen, —OH, —O—C₁-C₆alkyl, —OCF₃, —SH, —S—C₁-C₆alkyl, —NH₂, —NH(C₁-C₆alkyl), —N(C₁-C₆alkyl)₂, optionally substituted C₁-C₆alkyl, optionally substituted C₃-C₆cycloalkyl, optionally substituted C₁-C₆heteroalkyl, optionally substituted C₂-C₁₀heterocycloalkyl, optionally substituted aryl, or optionally substituted heteroaryl; each bb is 0, 1, or 2. In some embodiments, R^{2a} is halogen. In some embodiments, R^{2a} is —OCF₃. In some embodiments, bb is 0. In some embodiments bb is 1. In some embodiments, R^{2a} is halogen and bb is 1. In some embodiments, R^{2a} is —OCF₃ and bb is 1.

In some embodiments, the compounds of general structure exemplified by compound II-12 are prepared as shown in Scheme 2. In some embodiments, an appropriate indole aldehyde, such as compound II-1, is reductively alkylated with a Boc protected diamine, such as compound II-2, to provide amine II-3. In some embodiments, indole nitrogen of compound II-3 is converted to an aryl group using standard palladium and a suitable boronic acid coupling reagent, such as compound I-4, under suitable conditions to afford compound II-4. In some embodiments, compound II-4 is then functionalized at the 3 position with a suitable reagent, such as phosphorous oxychloride, to afford aldehyde II-5. In some embodiments, the aldehyde of compound II-5 is then reacted with an appropriate Homer-Emmons reagent to provide the nitrile II-6. In some embodiments, compound II-6 is then subjected under appropriate conditions to reduce both the double bond and nitrile with hydrogen to provide amine II-7. In some embodiments, amine II-7 is coupled with the Fmoc and Cbz protected amino acid, such as compound II-8, to provide compound II-9. In some embodiments, compound II-9 is subjected under appropriate conditions to remove to the Boc group to provide compound II-10. In some embodiments, compound II-10 is functionalized with an appropriate benzyl bromide, such as compound I-9, to afford a compound II-11. In some embodiments, compound II-11 is then reacted under suitable conditions, such as with piperidine, to remove the Fmoc group followed by appropriate reaction conditions to remove the Cbz group, such as hydrogenation at 1 atmosphere, to provide compound II-12.

Alternatively, the compound of II-11 is subjected under appropriate reaction conditions to provide compound II-13. In some instances, further reaction of compound II-13 under reductive amination or alkylation conditions with the appropriate reagent R—X followed by treatment under appropriate reaction conditions to cleave the Cbz group, such as Pd/C with hydrogen, provides compound II-14.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). In some embodiments, the compounds described herein are administered either alone or in combination with pharmaceutically acceptable carriers, excipients or diluents, in a pharmaceutical composition.

Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, or a pharmaceutically acceptable salt thereof, are used in the preparation of medicaments for the treatment of diseases or conditions in a mammal that would benefit from administration of any one of the compounds disclosed. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e. a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents. In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### Examples

The following examples are provided for illustrative purposes only and not to limit the scope of the claims provided herein.

All reactions are carried out under a nitrogen atmosphere under anhydrous conditions unless indicated otherwise. Anhydrous methylene chloride (DCM), tetrahydrofuran (THF) and N,N-dimethylformamide (DMF) are available from a vendor, such as Sigma-Aldrich. Typically, reactions are magnetically stirred and monitored by thin layer chromatography carried out by using pre-coated 0.25 mm silica plates containing a 254 nm fluorescence indicator. Flash chromatography is performed on an automatic flash chromatography system. Preparative thin layer chromatography is performed on 1 mm plates. Proton nuclear magnetic resonance spectra (¹H NMR, 300 MHz, 400 MHz, 500 MHz) and proton decoupled carbon nuclear magnetic resonance spectra (¹³C NMR, 100 MHz, 125 MHz) is obtained on a Bruker DPX 300, 400, or 500 MHz instruments in deuterochloroform (CDCl₃) with residual chloroform as internal standard.

As used above, and throughout the description of the invention, the following abbreviations, unless otherwise indicated, shall be understood to have the following meanings: DIPEA=diisopropylethyl amine; EtOAc=ethyl acetate; MeOH=methanol; DCE=1,2-dichloroethane; Pd(PPh₃)₄=Tetrakis(triphenylphos phine)palladium(0); Na₂SO₄=sodium sulfate; MgSO₄=magnesium sulfate; NaHCO₃=sodium bicarbonate; NH₄Cl=ammonium chloride; TFA=trifluoroacetic acid; HBTU=O-(Benzotriazol-1-yl)-N,N,N′,N′-tetramethyluronium hexafluorophosphate; HCl=hydrochloric acid; THF=tetrahydrofuran; and rt=room temperature.

### Example 1: Synthesis of 4-amino-N-[3-[5-[[4-[(2,6-dichlorophenyl)methyl]piperazin-1-yl]methyl]-3-[4-(trifluoromethoxy)phenyl]pyrrolo[2,3-b]pyridin-1-yl]propyl]piperidine-4-carboxamide (02-1)

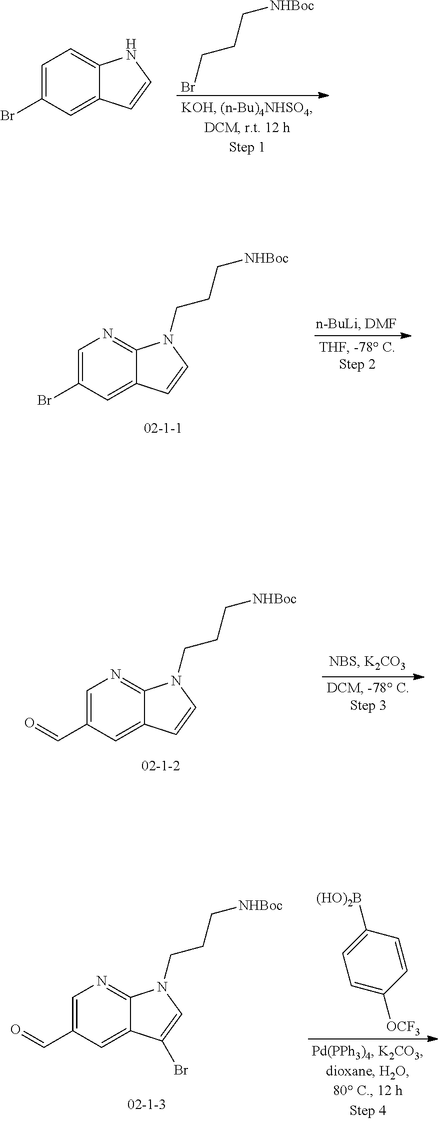

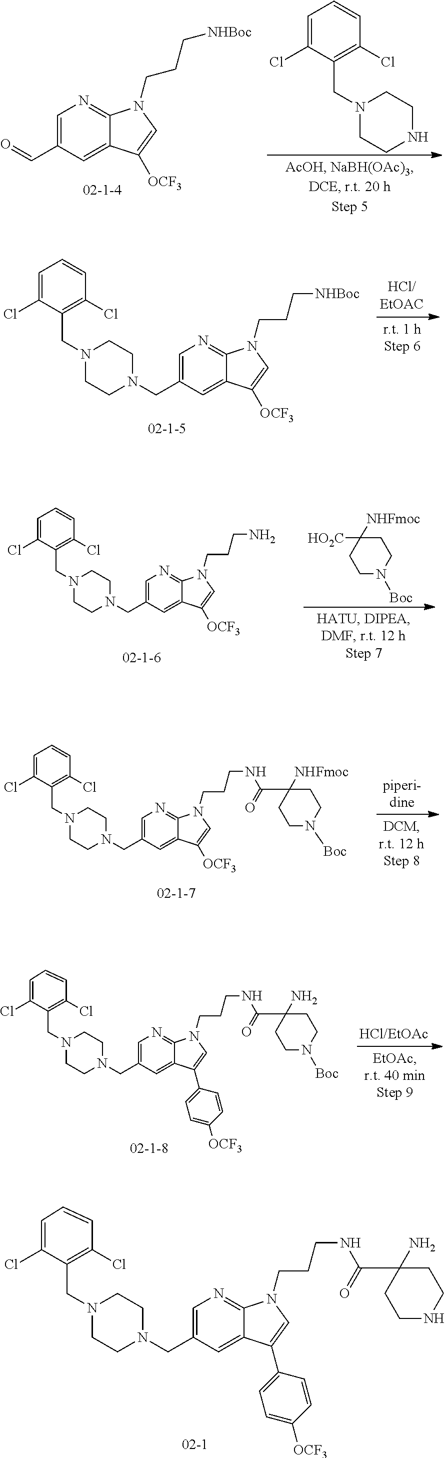

### Step 1: tert-butyl N-[3-(5-bromopyrrolo[2,3-b]pyridin-1-yl)propyl]carbamate (02-1-1)

To a mixture of 5-bromo-1H-pyrrolo[2,3-b]pyridine (10.0 g, 50.8 mmol, 1.0 eq) and KOH (7.1 g, 126.9 mmol, 2.5 eq) in DCM (200 mL) was added tetrabutylammonium hydrogen sulfate (17.2 g, 50.8 mmol, 1.0 eq) at room temperature, then the mixture was stirred for 0.5 h under N₂ before tert-Butyl N-(3-bromopropyl)carbamate (18.1 g, 76.1 mmol, 1.5 eq) was added. The resulting mixture was stirred at room temperature for 12 h, poured into water (100 mL), and the solution was extracted with DCM (30 mL*3). The organic layer was washed with brine (50 mL), dried over anhydrous Na₂SO₄, filtered and the filtrate was concentrated under reduced pressure. The residue was purified by column chromatography (SiO₂) to give compound 02-1-1 (14.0 g, 32.0 mmol, 63% yield). M+H⁺=354.1 (LCMS). ¹H NMR (400 MHz, DMSO-d₆): δ 8.27 (d, J=2.21 Hz, 1H), 8.17 (d, J=1.76 Hz, 1H), 7.63 (d, J=3.09 Hz, 1H), 6.88 (br. s., 1H), 6.44 (d, J=3.09 Hz, 1H), 4.23 (t, J=6.84 Hz, 2H), 2.89 (q, J=6.17 Hz, 2H), 1.94-1.81 (m, 2H), 1.44-1.25 (m, 9H).

### Step 2: tert-butyl N-[3-(5-formylpyrrolo[2,3-b]pyridin-1-yl)propyl]carbamate (02-1-2)

To a mixture of compound 02-1-1 (4.50 g, 12.7 mmol, 1.0 eq) in THF (100 mL) was added n-BuLi (2.5 M, 10.2 mL, 2.0 eq) dropwise at −78° C. under N₂. Then the mixture was stirred at −78° C. for 0.5 h. DMF (1.11 g, 15.2 mmol, 1.2 mL, 1.2 eq) was added to the mixture dropwise at −78° C. under N₂. After the addition, the mixture was stirred at −78° C. for 2 h. The reaction mixture was quenched by water (200 mL), and the mixture was extracted with EtOAc (60 mL*3). The organic layers were washed with brine (100 mL), dried over anhydrous Na₂SO₄, filtered and the filtrate was concentrated. The residue was purified by column chromatography (SiO₂) to give compound 02-1-2 (4.30 g, 14.2 mmol, 2 batches in parallel, 56% yield). ¹H NMR (400 MHz, DMSO-d₆): δ 10.14-10.04 (m, 1H), 8.79 (d, J=1.76 Hz, 1H), 8.55-8.44 (m, 1H), 7.76 (d, J=3.53 Hz, 1H), 6.91 (br. s., 1H), 6.70 (d, J=3.53 Hz, 1H), 4.32 (t, J=7.06 Hz, 2H), 2.92 (q, J=6.17 Hz, 2H), 1.91 (quin, J=6.73 Hz, 2H), 1.43-1.32 (m, 9H).

### Step 3: tert-butyl N-[3-(3-bromo-5-formyl-pyrrolo[2,3-b]pyridin-1-yl)propyl]carbamate (02-1-3)

To a mixture of compound 02-1-2 (5.00 g, 16.5 mmol, 1.0 eq) and K₂CO₃ (3.4 g, 24.7 mmol, 1.5 eq) in DCM (60 mL) was added NBS (2.64 g, 14.8 mmol, 0.9 eq) in portions at −78° C. After the addition, the reaction mixture was stirred at −78° C. for 0.5 h, and poured into water (60 mL). The mixture was extracted with DCM (30 mL*3). The combined organic layers were washed with brine (40 mL), dried over anhydrous Na₂SO₄, filtered and the filtrate was concentrated. The residue was purified by column chromatography (SiO₂) to give compound 02-1-3 (5.50 g, 14.4 mmol, 87% yield). M+H⁺=382.2 (LSMC) ¹H NMR (DMSO-d₆): δ 10.15 (s, 1H), 8.95-8.77 (m, 1H), 8.47-8.31 (m, 1H), 8.04 (s, 1H), 6.90 (br. s., 1H), 4.32 (t, J=6.62 Hz, 2H), 2.91 (d, J=5.73 Hz, 2H), 1.99-1.85 (m, 2H), 1.44-1.28 (m, 9H).

### Step 4: tert-butyl N-[3-[5-formyl-3-[4-(trifluoromethoxy)phenyl]pyrrolo[2,3-b]pyridin-1-yl]propyl]carbamate (02-1-4)

A mixture of compound 02-1-3 (5.00 g, 13.1 mmol, 1.0 eq), [4-(trifluoromethoxy)phenyl]boronic acid (4.0 g, 19.6 mmol, 1.5 eq), Pd(PPh₃)₄(756.0 mg, 654.2 μmol, 0.05 eq) and K₂CO₃ (3.62 g, 26.2 mmol, 2.0 eq) in dioxane (100 mL) and H₂O (10 mL) was degassed and then heated to 80° C. for 12 h under N₂. The reaction mixture was cooled down to room temperature, diluted with water (200 mL) and extracted with EtOAc (100 mL*3). The combined organic layers were washed with brine (150 mL), dried over anhydrous Na₂SO₄, then filtered and the filtrate was concentrated. The residue was purified by column chromatography (SiO₂) to give compound 02-1-4 (5.50 g, 11.9 mmol, 91% yield). ¹H NMR (400 MHz, CDCl₃): δ 10.16 (s, 1H), 8.87 (d, J=1.76 Hz, 1H), 8.66 (d, J=1.76 Hz, 1H), 7.72-7.61 (m, 3H), 7.57-7.52 (m, 1H), 7.50-7.43 (m, 1H), 7.33 (d, J=8.38 Hz, 2H), 4.47 (t, J=6.39 Hz, 2H), 3.12 (d, J=5.73 Hz, 2H), 2.14-2.06 (m, 2H), 1.51-1.42 (m, 9H).

### Step 5: tert-butyl N-[3-[5-[[4-[(2,6-dichlorophenyl)methyl]piperazin-1-yl]methyl]-3-[4-(trifluoromethoxy)phenyl]pyrrolo[2,3-b]pyridin-1-yl]propyl]carbamate (02-1-5)

To a mixture of compound 02-1-4 (900.0 mg, 1.94 mmol, 1.0 eq) in DCE (20 mL) was added 1-[(2,6-dichlorophenyl)methyl]piperazine (571.0 mg, 2.33 mmol, 1.2 eq) and AcOH (116.5 mg, 1.94 mmol, 111.0 μL, 1.0 eq), then the mixture was stirred at room temperature for 12 h. NaBH(OAc)₃ (822.0 mg, 3.88 mmol, 2.0 eq) was added in portions under N₂. After the addition, the reaction mixture was stirred at room temperature for 8 h before water (50 mL) was added. The solution was basified with Na₂CO₃ powder to pH=8. Then the solution was extracted with DCM (20 mL*4). The combined organic layers were washed with brine (40 mL), dried over anhydrous Na₂SO₄, filtered and the filtrate was concentrated to give a residue which was purified by column chromatography (SiO₂) to give compound 02-1-5 (1.00 g, 1.44 mmol, 74% yield). ¹H NMR (400 MHz, CDCl₃): δ 8.22 (d, J=1.32 Hz, 1H), 8.01 (s, 1H), 7.55 (d, J=8.82 Hz, 2H), 7.33 (s, 1H), 7.28-7.18 (m, 4H), 7.07-7.01 (m, 1H), 5.38 (br. s., 1H), 4.37-4.27 (m, 2H), 3.68 (s, 2H), 3.59-3.50 (m, 2H), 3.00 (d, J=5.73 Hz, 2H), 2.54 (br. s., 4H), 2.40 (br. s., 4H), 2.01-1.98 (m, 1H), 1.96-1.93 (m, 1H), 1.38 (s, 9H).

### Step 6: 3-[5-[[4-[(2,6-dichlorophenyl)methyl]piperazin-1-yl]methyl]-3-[4-(trifluoromethoxy)phenyl]pyrrolo[2,3-b]pyridin-1-yl]propan-1-amine (02-1-6)

To a mixture of compound 02-1-5 (1.00 g, 1.44 mmol, 1.0 eq) in EtOAc (10 mL) was added HCl/EtOAc (4 M, 20 mL) at room temperature, then the reaction mixture was stirred for 1 h. The reaction mixture was added to water (40 mL) and the organic layer was separated and discarded. The aqueous phase was basified with Na₂CO₃ powder to pH=8 and extracted with DCM (20 mL*3). The combined DCM layers were washed with brine (30 mL), dried over anhydrous Na₂SO₄, filtered and the filtrate was concentrated to give compound 02-1-6 (700.0 mg, 1.18 mmol, 82% yield). M+H⁺=592.3 (LCMS). ¹H NMR (400 MHz, MeOD): δ 8.94 (br. s., 1H), 8.68 (br. s., 1H), 8.05 (s, 1H), 7.92 (d, J=7.89 Hz, 2H), 7.63-7.57 (m, 2H), 7.56-7.50 (m, 1H), 7.40 (d, J=7.89 Hz, 2H), 4.84 (s, 2H), 4.78 (br. s., 2H), 4.57 (br. s., 2H), 3.95 (br. s., 4H), 3.83 (br. s., 4H), 3.00 (br. s., 2H), 2.32 (br. s., 2H).

### Step 7: tert-butyl 4-[3-[5-[[4-[(2,6-dichlorophenyl)methyl]piperazin-1-yl]methyl]-3-[4-(trifluoromethoxy)phenyl]pyrrolo[2,3-b]pyridin-1-yl]propylcarbamoyl]-4-(9H-fluoren-9-ylmethoxycarbonylamino)piperidine-1-carboxylate (02-1-7)

To a mixture of 1-tert-butoxycarbonyl-4-(9H-fluoren-9-ylmethoxycarbonylamino)piperidine-4-carboxylic acid (433.0 mg, 928.3 μmol, 1.1 eq) and DIPEA (218.0 mg, 1.69 mmol, 294.6 μL, 2.0 eq) in DMF (10 mL) was added HATU (353.0 mg, 928.3 μmol, 1.1 eq), then the mixture was stirred at room temperature for 0.5 h. A solution of compound 02-1-6 (500.0 mg, 843.9 μmol, 1.0 eq) in DMF (5 mL) was added to the mixture at room temperature. After the addition, the reaction mixture was stirred at room temperature for 12 h. After the reaction mixture was added to water (50 mL), the precipitated white solid was filtered and the solid was washed with water (15 mL*2). Then the collected solid was dissolved in DCM (40 mL), dried over anhydrous Na₂SO₄, filtered and the filtrate was concentrated. The residue was purified by column chromatography (SiO₂) to give compound 02-1-7 (690.0 mg, 662.8 μmol, 79% yield). ¹H NMR (400 MHz, CDCl₃): δ 8.17 (br. s., 1H), 7.99 (s, 2H), 7.69-7.61 (m, 2H), 7.52 (d, J=8.82 Hz, 2H), 7.45 (d, J=7.50 Hz, 2H), 7.33-7.14 (m, 8H), 7.09-6.99 (m, 1H), 5.02 (br. s., 1H), 4.32 (d, J=16.32 Hz, 4H), 4.09 (t, J=6.39 Hz, 1H), 3.85 (br. s., 2H), 3.69-3.58 (m, 2H), 3.48 (br. s., 2H), 3.08-2.83 (m, 4H), 2.60-2.25 (m, 7H), 2.17-1.84 (m, 6H), 1.72-1.55 (m, 2H), 1.51-1.33 (m, 9H).

### Step 8: tert-butyl 4-amino-4-[3-[5-[[4-[(2,6-dichlorophenyl)methyl]piperazin-1-yl]methyl]-3-[4-(trifluoromethoxy)phenyl]pyrrolo[2,3-b]pyridin-1-yl]propylcarbamoyl]piperidine-1-carboxylate (02-1-8)

To a mixture of compound 02-1-7 (630.0 mg, 605.2 μmol, 1.0 eq) in DCM (15 mL) piperidine (412.3 mg, 4.84 mmol, 479.4 μL, 8.0 eq) was added at room temperature. The mixture was stirred at room temperature under N₂ for 4 h, and concentrated under reduced pressure to give the crude product. The crude product was purified by column chromatography (SiO₂) to give compound 02-1-8 (370.0 mg, 451.9 μmol, 75% yield). M+H⁺=818.3 (LCMS). ¹H NMR (400 MHz, CDCl₃): δ 8.21 (s, 1H), 8.13 (t, J=5.73 Hz, 1H), 8.03 (s, 1H), 7.55 (d, J=8.82 Hz, 2H), 7.35 (s, 1H), 7.27-7.17 (m, 4H), 7.09-7.01 (m, 1H), 4.31 (t, J=6.17 Hz, 2H), 3.86 (br. s., 2H), 3.68 (s, 2H), 3.59 (s, 2H), 3.19-2.94 (m, 4H), 2.55 (br. s., 4H), 2.44 (br. s., 4H), 2.12-1.96 (m, 4H), 1.60-1.46 (m, 2H), 1.45-1.34 (m, 9H), 1.34-1.23 (m, 2H).

### Step 9: 4-amino-N-[3-[5-[[4-[(2,6-dichlorophenyl)methyl]piperazin-1-yl]methyl]-3-[4-(trifluoromethoxy)phenyl]pyrrolo[2,3-b]pyridin-1-yl]propyl]piperidine-4-carboxamide (02-1)

To a mixture of compound 02-1-8 (270.0 mg, 329.8 μmol, 1.0 eq) in EtOAc (5 mL) was added HCl/EtOAc (4 M, 20 mL) at room temperature, and the reaction mixture was stirred for 40 min. During the course of the reaction a solid precipitated. The reaction mixture was filtered and the filter cake was washed with DCM (10 mL*3). The solid was collected and dried under reduced pressure to give compound 02-1 (236.7 mg, 299.1 μmol, 91% yield, HCl). M+H⁺=718.4 (LCMS). ¹H NMR (400 MHz, MeOD): δ 9.02 (s, 1H), 8.77 (s, 1H), 8.18 (s, 1H), 7.94 (d, J=8.82 Hz, 2H), 7.62-7.57 (m, 2H), 7.56-7.49 (m, 1H), 7.40 (d, J=7.94 Hz, 2H), 4.81 (d, J=10.14 Hz, 4H), 4.56 (t, J=6.84 Hz, 2H), 3.95 (br. s., 4H), 3.84 (br. s., 4H), 3.55-3.42 (m, 4H), 3.37 (t, J=6.17 Hz, 2H), 2.81-2.70 (m, 2H), 2.33-2.19 (m, 4H).

### Example 2: Synthesis of N-[3-[5-[[4-[(2,6-dichlorophenyl)methyl]piperazin-1-yl]methyl]-3-[4-(trifluoro methoxy)phenyl]pyrrolo[2,3-b]pyridin-1-yl]propyl]piperazine-2-carboxamide (02-2)

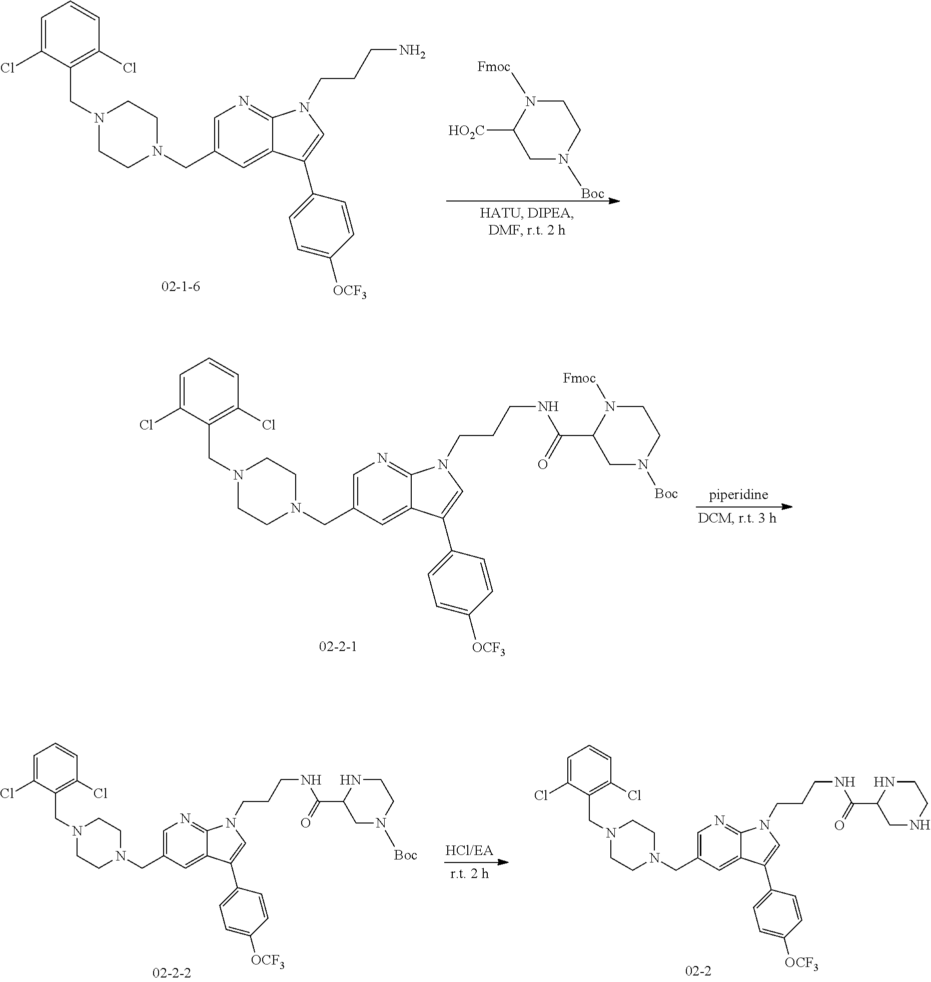

Compound 02-2 was prepared from compound 02-1-6 according to the procedures described in steps 7-9 in the synthesis of compound 02-1.

02-2-1 ¹H NMR (CDCl₃, 400 MHz): δ 8.17 (br. s., 1H), 8.00 (br. s., 1H), 7.65 (d, J=7.06 Hz, 2H), 7.56-7.32 (m, 4H), 7.29 (br. s., 3H), 7.24-7.10 (m, 6H), 7.09-6.99 (m, 1H), 4.71-4.34 (m, 5H), 4.19 (d, J=14.55 Hz, 3H), 3.90 (br. s., 2H), 3.68-3.31 (m, 5H), 3.15-2.87 (m, 4H), 2.55-2.16 (m, 7H), 1.88 (br. s., 2H), 1.38 (s, 9H).

02-2 ¹H NMR (MeOD, 400 MHz): δ 9.09 (d, J=1.3 Hz, 1H), 8.82 (d, J=1.3 Hz, 1H), 8.19 (s, 1H), 7.95 (d, J=8.4 Hz, 2H), 7.66-7.57 (m, 2H), 7.56-7.50 (m, 1H), 7.41 (br d, J=7.9 Hz, 2H), 4.83 (br d, J=8.8 Hz, 4H), 4.66-4.49 (m, 3H), 4.07 (br dd, J=3.5, 13.7 Hz, 1H), 3.97 (br d, J=4.4 Hz, 3H), 3.86 (br s, 3H), 3.80-3.68 (m, 2H), 3.63-3.43 (m, 3H), 3.42-3.32 (m, 2H), 3.31-3.22 (m, 2H), 2.30-2.18 (m, 2H). M+H⁺=704.3 (LCMS).

### Example 3: Synthesis of 3-[5-[[4-[(2-chlorophenyl)methyl]piperazin-1-yl]methyl]-3-[4-(trifluoromethoxy)phenyl]indazol-1-yl]propan-1-amine (02-5)

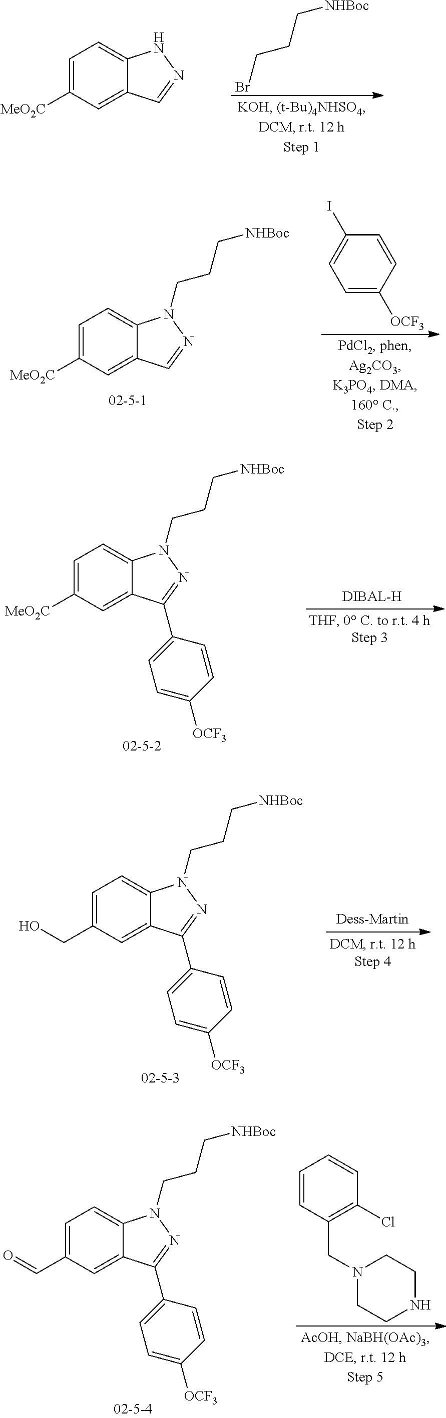

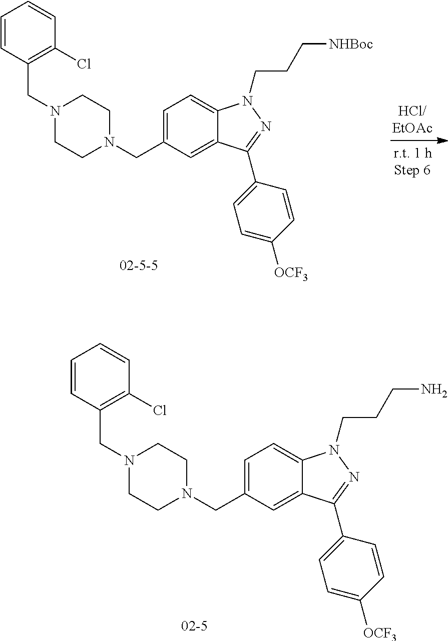 [SMILES: CC(C)(C)OC(=O)NCCCn1nc(-c2ccc(OC(F)(F)F)cc2)c2cc(CN3CCN(Cc4ccccc4Cl)CC3)ccc21 ; NCCCn1nc(-c2ccc(OC(F)(F)F)cc2)c2cc(CN3CCN(Cc4ccccc4Cl)CC3)ccc21]

### Step 1: methyl 1-[3-(tert-butoxycarbonylamino)propyl]indazole-5-carboxylate (02-5-1)

To a solution of methyl 1H-indazole-5-carboxylate (10.0 g, 56.8 mmol, 1.0 eq) and KOH (7.96 g, 141.9 mmol, 2.5 eq) in DCM (200 mL) was added tetrabutylammonium hydrogen sulfate (19.3 g, 56.76 mmol, 1.0 eq). The mixture was stirred at room temperature for 0.5 h. Then tert-butyl N-(3-bromopropyl)carbamate (20.3 g, 85.1 mmol, 1.5 eq) was added to the mixture. The mixture was stirred at room temperature for 12 h under N2. The mixture was poured into water (200 mL), and extracted with DCM 400 mL (200 mL*2). The combined organic layers were washed with brine 200 mL (100 mL*2), dried over anhydrous Na2SO4, filtered and concentrated under reduced pressure. The residue was purified by column chromatography (SiO2) to give compound 02-5-1 (10.0 g, 29.4 mmol, 52% yield) and byproduct methyl 2-[3-(tert-butoxycarbonylamino)propyl]indazole-5-carboxylate (7.0 g, 18.7 mmol, 33% yield). 1H NMR (MeOD, 400 MHz): δ 8.37 (s, 1H), 8.04 (s, 1H), 7.90 (dd, J=1.6 Hz, J=9.2 Hz, 1H), 7.48 (d, J=8.8 Hz, 1H), 4.35 (t, J=7.2 Hz, 2H), 3.81 (s, 3H), 2.95-2.92 (m, 2H), 2.00-1.93 (m, 2H), 1.30 (s, 9H).

### Step 2: methyl 1-[3-(tert-butoxycarbonylamino)propyl]-3-[4-(trifluoromethoxy)phenyl]indazole-5-carboxylate (02-5-2)

To a solution of compound 02-5-1 (1.00 g, 3.0 mmol, 1.0 eq) in DMA (5 mL) was added 1-iodo-4-(trifluoromethoxy)benzene (1.73 g, 6.0 mmol, 939 μL, 2.0 eq), PdCl₂ (10.6 mg, 60.0 μmol, 0.02 eq), 1,10-phenanthroline (10.8 mg, 60.0 μmol, 0.02 eq), Ag₂CO₃ (1.24 g, 4.50 mmol, 204 μL, 1.5 eq) and K₃PO₄ (1.27 g, 6.0 mmol, 2.0 eq). The suspension was degassed under vacuum and purged with N₂ several times. The mixture was stirred at 150° C. for 12 h. The reaction mixture was poured into H₂O (100 mL), filtered through Celite, and washed with EtOAc (50 mL). The filtrate was extracted with EtOAc (100 mL*3). The combined organic layers were washed with brine (100 mL*2), dried over anhydrous Na₂SO₄, filtered and concentrated under reduced pressure. The residue was purified by column chromatography (SiO₂) to give compound 02-5-2 (300.0 mg, 608 μmol, 20% yield).

### Step 3: tert-butyl N-[3-[5-(hydroxymethyl)-3-[4-(trifluoromethoxy)phenyl]indazol-1-yl]propyl]carbamate (02-5-3)

To a solution of compound 02-5-2 (300.0 mg, 607.9 μmol, 1.0 eq) in THF (5 mL) was added DIBAL-H (1 M, 2.43 mL, 4.0 eq) at 0° C. The mixture was stirred at room temperature for 4 h, quenched with H₂O (10 mL) at 0° C., and then extracted with ethyl acetate (20 mL*3). The combined organic layers were washed with brine (20 mL*2), dried over anhydrous Na₂SO₄, filtered, and concentrated under reduced pressure to give compound 02-5-3 (250.0 mg, crude), which was used in the next step without further purification.

### Step 4: tert-butyl N-[3-[5-formyl-3-[4-(trifluoromethoxy)phenyl]indazol-1-yl]propyl]carbamate (02-5-4)

To a solution of the compound 02-5-3 (250.0 mg, 537.1 μmol, 1.0 eq) in DCM (5 mL) was added Dess-Martin (455.6 mg, 1.07 mmol, 332.6 μL, 2.0 eq). The mixture was stirred at room temperature for 12 h, diluted with DCM (10 mL), filtered and concentrated under reduced pressure to give compound 02-5-4 (200.0 mg, crude), which was used in the next step without further purification.

### Step 5: tert-butyl N-[3-[5-[[4-[(2-chlorophenyl)methyl]piperazin-1-yl]methyl]-3-[4-(trifluoromethoxy)phenyl]indazol-1-yl]propyl]carbamate (02-5-5)

To a solution of the compound 02-5-4 (250.0 mg, 539.4 μmol, 1.0 eq) in DCE (5 mL) was added 1-[(2-chlorophenyl)methyl]piperazine (125.0 mg, 593.4 μmol, 1.1 eq), AcOH (32.4 mg, 539.4 μmol, 30.9 μL, 1.0 eq) and NaBH(OAc)₃ (343.0 mg, 1.62 mmol, 3.0 eq). The mixture was stirred at room temperature for 12 h, poured into H₂O (10 mL) and extracted with DCM (20 mL*2). The combined organic layers were washed with Na₂CO₃ (aq., 20 mL*2) and brine (20 mL*2), dried over anhydrous Na₂SO₄, filtered and concentrated under reduced pressure. The residue was purified by prep-TLC (SiO₂) to give compound 02-5-5 (380.0 mg, 271.4 μmol, 50% yield). M+H⁺=658.3 (LCMS).

### Step 6: 3-[5-[[4-[(2-chlorophenyl)methyl]piperazin-1-yl]methyl]-3-[4-(trifluoromethoxy)phenyl]indazol-1-yl]propan-1-amine (02-5)

To a solution of compound 02-5-5 (100.0 mg, 151.9 μmol, 1.0 eq) in EtOAc (2 mL) was added HCl/EtOAc (4 M, 2.0 mL, 52.7 eq). The mixture was stirred at room temperature for 1 h. The reaction mixture was concentrated under reduced pressure to give a residue. The crude product was washed with ether (2 mL) to give compound 02-5 (12.0 mg, 18.4 μmol, 12% yield, HCl). M+H⁺=612.4 (LCMS). ¹H NMR (MeOD, 400 MHz): δ 8.41 (s, 1H), 8.15 (br d, J=8.4 Hz, 2H), 7.81 (br d, J=8.8 Hz, 1H), 7.74-7.68 (m, 2H), 7.54 (br d, J=7.5 Hz, 1H), 7.50-7.40 (m, 4H), 4.68-4.59 (m, 4H), 4.44 (br s, 2H), 3.59 (br s, 8H), 3.07-3.01 (m, 2H), 2.37-2.29 (m, 2H).

### Example 4: Synthesis of 3-(6-((4-(2,6-dichlorobenzyl)piperazin-1-yl)methyl)-1-(4-(trifluoromethoxy)phenyl)-1H-indol-3-yl)propan-1-amine (03-1-8)

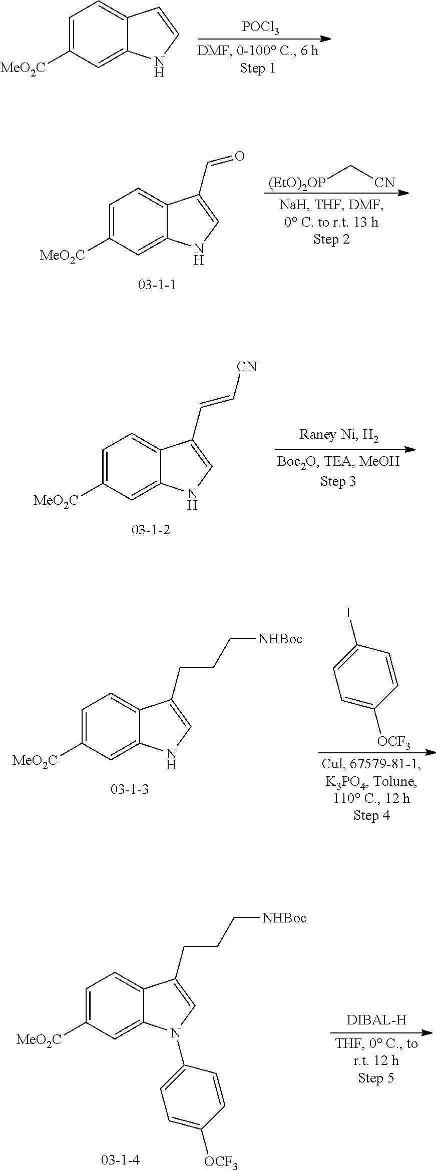

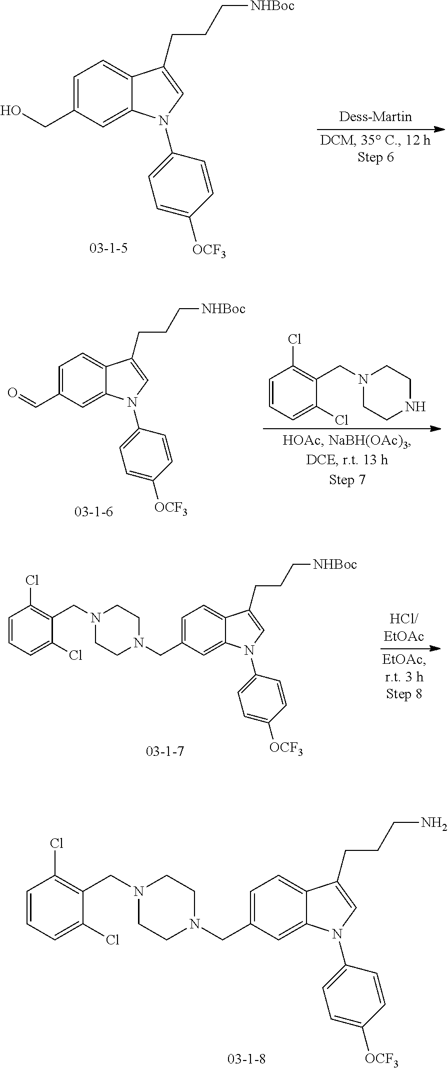 [SMILES: CC(C)(C)OC(=O)NCCCc1cn(-c2ccc(OC(F)(F)F)cc2)c2cc(CO)ccc12 ; CC(C)(C)OC(=O)NCCCc1cn(-c2ccc(OC(F)(F)F)cc2)c2cc(C=O)ccc12 ; Clc1cccc(Cl)c1CN1CCNCC1 ; CC(C)(C)OC(=O)NCCCc1cn(-c2ccc(OC(F)(F)F)cc2)c2cc(CN3CCN(Cc4c(Cl)cccc4Cl)CC3)ccc12 ; NCCCc1cn(-c2ccc(OC(F)(F)F)cc2)c2cc(CN3CCN(Cc4c(Cl)cccc4Cl)CC3)ccc12]

### Step 1: Methyl 3-formyl-1H-indole-6-carboxylate (03-1-1)

To DMF (20.9 g, 285.4 mmol, 22.0 mL, 1.0 eq) in a three-necked flask equipped with a stirrer, a thermometer at 0° C., was slowly added dropwise POCl₃ (43.8 g, 285.4 mmol, 26.5 mL, 1.0 eq). After the addition, the mixture was stirred for 1 h at 0° C. Then methyl 1H-indole-6-carboxylate (50.0 g, 285.4 mmol, 1.0 eq) in DMF (600 mL) was added to the reaction mixture dropwise at 0° C. The mixture was allowed to warm to room temperature and stirred for 3 h with solid precipitating out. Then H₂O (200 mL) was added to the mixture and the solution was basified with NaOH to pH=8-9. The basified solution was stirred at 100° C. for additional 2 h. Water (2 L) was added to the mixture, and the resulting suspension was stirred for 0.5 h, and filtered. The filter cake was washed with H₂O (200 mL) and dried under reduced pressure to give methyl 03-1-1 (50.0 g, 86%). ¹H NMR (DMSO, 400 MHz): δ 12.43 (br. s., 1H), 10.05-9.92 (m, 1H), 8.56-8.43 (m, 1H), 8.23-8.09 (m, 2H), 7.87-7.79 (m, 1H), 3.97-3.77 (m, 3H).

### Step 2: Methyl 3-[(E)-2-cyanovinyl]-1H-indole-6-carboxylate (03-1-2)

To a stirred solution of 2-diethoxyphosphorylacetonitrile (61.0 g, 344.5 mmol, 1.4 eq) in THF (90 mL) and DMF (90 mL) was added NaH (13.8 g, 344.5 mmol, 1.4 eq) at 0° C. After 1 h, compound 03-1-1 (50.0 g, 246.1 mmol, 1.0 eq) in DMF (200 mL) was added to the mixture at 10° C. The reaction mixture was stirred at room temperature for 12 h and poured into H₂O (1 L). The suspension was stirred for 0.5 h and filtered. The solid was suspended in a mixture of petroleum ether/EtOAc (1 L, 10:1), stirred for 0.5 h and then filtered again. The collected solid was dried under reduced pressure to give compound 03-1-2 (66.0 g, crude), which was used into the next step without further purification. M+H⁺=227.1 (LCMS). ¹H NMR (DMSO-d₆, 400 MHz): δ 8.16-8.07 (m, 2H), 8.01 (d, J=8.38 Hz, 1H), 7.80-7.72 (m, 2H), 6.18-6.08 (m, 1H), 3.86 (s, 3H)

### Step 3: Methyl 3-[3-(tert-butoxycarbonylamino)propyl]-1H-indole-6-carboxylate (03-1-3)

To a solution of the compound 03-1-2 (11.0 g, 48.6 mmol, 1.0 eq) in MeOH (100 mL) and TEA (14.8 g, 145.9 mmol, 20.2 mL, 3.0 eq) was added Raney-Ni (10 g) and Boc₂O (31.8 g, 145.9 mmol, 33.5 mL, 3.0 eq) under N₂. The suspension was degassed under vacuum and purged with H₂ several times. The mixture was stirred under H₂ (30 Psi) at room temperature for 7 h. The mixture was filtered through Celite and concentrated to give a residue. The residue was purified by column chromatography (SiO₂) to give compound 03-1-3 (35.0 g, 6 batches in parallel, 43% for 2 steps). M+H⁺=233.2 (LCMS). ¹H NMR (DMSO-d₆, 400 MHz): δ 11.19 (br. s., 1H), 8.01 (s, 1H), 7.62-7.55 (m, 2H), 7.39 (d, J=2.13 Hz, 1H), 6.86 (br. s., 1H), 3.84 (s, 3H), 3.02-2.91 (m, 2H), 2.68 (t, J=7.47 Hz, 2H), 1.74 (quin, J=7.25 Hz, 2H), 1.41-1.35 (m, 9H).

### Step 4: Methyl 3-[3-(tert-butoxycarbonylamino)propyl]-1-[4-(trifluoromethoxy)phenyl]indole-6-carboxylate (03-1-4)

A mixture of compound 03-1-3 (2.50 g, 7.52 mmol, 1.0 eq), 1-iodo-4-(trifluoromethoxy)benzene (3.25 g, 11.3 mmol, 1.5 eq), CuI (143.2 mg, 752.1 μmol, 0.1 eq), (1S,2S)—N1,N2-dimethylcyclohexane-1,2-diamine (321.0 mg, 2.26 mmol, 0.3 eq) and K₃PO₄ (3.19 g, 15.0 mmol, 2.0 eq) in toluene (3.0 mL) was heated at 110° C. for 12 h under N₂. The reaction mixture was diluted with H₂O (100 mL) and extracted with ethyl acetate (80 mL*4). The combined organic layers were washed with brine (20 mL*3), dried over anhydrous Na₂SO₄, filtered and concentrated under reduced pressure to give a residue. The residue was purified by column chromatography (SiO₂) to give compound 03-1-4 (3.00 g, 74%). M+H⁺=393.2 (LCMS).

Steps 5-8 were carried out according to the procedures described in Steps 3-6 in the synthesis of compound 02-5.

### Step 5: tert-Butyl N-[3-[6-(hydroxymethyl)-1-[4-(trifluoromethoxy)phenyl]indol-3-yl]propyl]carbamate (03-1-5)

Compound 03-1-5 was sythesized from compound 03-1-4 following the procedure described for the sythesis of compound 02-5-3. M+Na⁺=487.2 (LCMS). ¹H NMR (MeOD, 400 MHz): δ 7.69 (d, J=8.82 Hz, 2H), 7.61-7.50 (m, 4H), 7.44 (s, 1H), 7.09 (d, J=8.38 Hz, 1H), 6.90 (br. s., 1H), 5.16-5.06 (m, 1H), 4.59 (d, J=5.73 Hz, 2H), 3.02 (q, J=6.62 Hz, 2H), 2.72 (t, J=7.28 Hz, 2H), 1.80 (quin, J=7.06 Hz, 2H), 1.42-1.32 (m, 9H).

### Step 6: tert-Butyl N-[3-[6-formyl-1-[4-(trifluoromethoxy)phenyl]indol-3-yl]propyl]carbamate (03-1-6)

Compound 03-1-6 was sythesized from compound 03-1-5 following the procedure described for the sythesis of compound 02-5-4. M+H⁺=463.2 (LCMS). ¹H NMR (DMSO-d₆, 400 MHz): δ 10.05-9.98 (m, 1H), 8.10 (s, 1H), 7.82-7.76 (m, 4H), 7.71-7.57 (m, 4H), 6.91 (t, J=5.46 Hz, 1H), 3.02 (q, J=6.53 Hz, 2H), 2.77 (t, J=7.40 Hz, 2H), 1.81 (quin, J=7.15 Hz, 2H), 1.37 (s, 9H).

### Step 7: tert-Butyl N-[3-[6-[[4-[(2,6-dichlorophenyl)methyl]piperazin-1-yl]methyl]-1-[4-(trifluoro methoxy)phenyl]indol-3-yl]propyl]carbamate (03-1-7)

Compound 03-1-7 was sythesized from compound 03-1-6 following the procedure described for the sythesis of compound 02-5-5. M+H⁺=691.3 (LCMS). ¹H NMR (DMSO-d₆, 400 MHz): δ 7.68 (d, J=8.82 Hz, 2H), 7.59-7.52 (m, 3H), 7.43 (d, J=7.50 Hz, 4H), 7.34-7.27 (m, 1H), 7.09 (d, J=7.94 Hz, 1H), 6.93-6.85 (m, 1H), 3.64 (s, 2H), 3.52 (br. s., 2H), 3.01 (q, J=6.47 Hz, 2H), 2.70 (t, J=7.06 Hz, 2H), 2.33 (br. s., 4H), 2.45 (br. s., 5H), 1.79 (dt, J=14.11, 7.06 Hz, 2H), 1.44-1.29 (m, 9H).

### Step 8: 3-[6-[[4-[(2,6-dichlorophenyl)methyl]piperazin-1-yl]methyl]-1-[4-(trifluoromethoxy)phenyl]indol-3-yl]propan-1-amine (03-1-8)

Compound 03-1-8 was sythesized from compound 03-1-7 following the procedure described for the sythesis of compound 02-5. M+H⁺=591.3 (LCMS). ¹H NMR (MeOD, 400 MHz): δ 7.65 (d, J=8.53 Hz, 3H), 7.60 (br. s., 1H), 7.49 (d, J=8.78 Hz, 2H), 7.38 (d, J=8.03 Hz, 3H), 7.28-7.22 (m, 1H), 7.19 (d, J=8.16 Hz, 1H), 3.89-3.80 (m, 4H), 3.05-2.99 (m, 2H), 2.94 (t, J=7.03 Hz, 2H), 2.69 (br. s., 8H), 2.15-2.05 (m, 2H).

### Example 5: Synthesis of 4-amino-N-[3-[6-[[4-[(2,6-dichlorophenyl)methyl]piperazin-1-yl]methyl]-1-[4-(trifluoromethoxy)phenyl]indol-3-yl]propyl]piperidine-4-carboxamide (03-1)

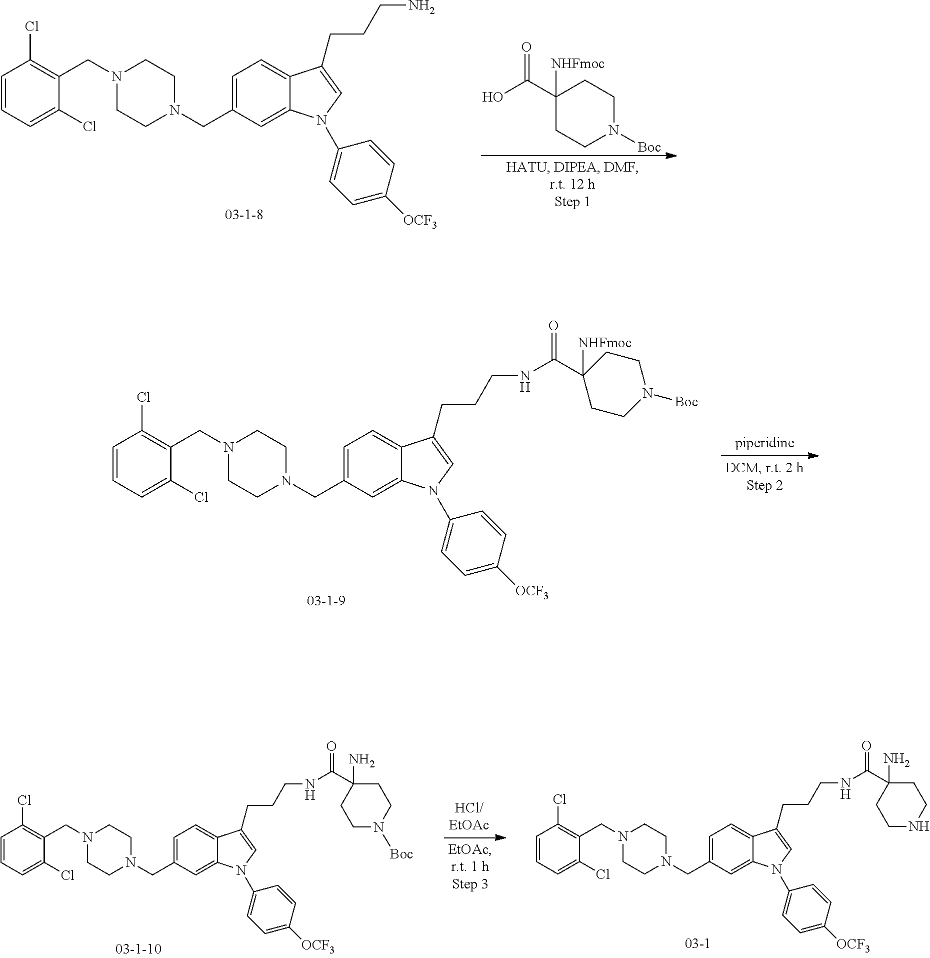

### 4-amino-N-[3-[6-[[4-[(2,6-dichlorophenyl)methyl]piperazin-1-yl]methyl]-1-[4-(trifluoromethoxy)phenyl]indol-3-yl]propyl]piperidine-4-carboxamide (03-1)

Compound 03-1 was synthesized from compound 03-1-8 according to the procedures described in Steps 7-9 in the synthesis of compound 02-1. M+H⁺=717.3 (LCMS). ¹H NMR (MeOD, 400 MHz, HCl salt): δ 7.86 (s, 1H), 7.81-7.73 (m, 3H), 7.57-7.44 (m, 6H), 7.35 (d, J=7.94 Hz, 1H), 4.55 (s, 4H), 3.62 (br. s., 7H), 3.52-3.33 (m, 7H), 2.90 (t, J=7.06 Hz, 2H), 2.76-2.67 (m, 2H), 2.24-2.15 (m, 2H), 2.09-2.00 (m, 2H).

### Example 6: Synthesis of tert-butyl 3-[3-[6-[[4-[(2,6-dichlorophenyl)methyl]piperazin-1-yl]methyl]-1-[4-(trifluoromethoxy)phenyl]indol-3-yl]propylcarbamoyl]piperazine (03-2)

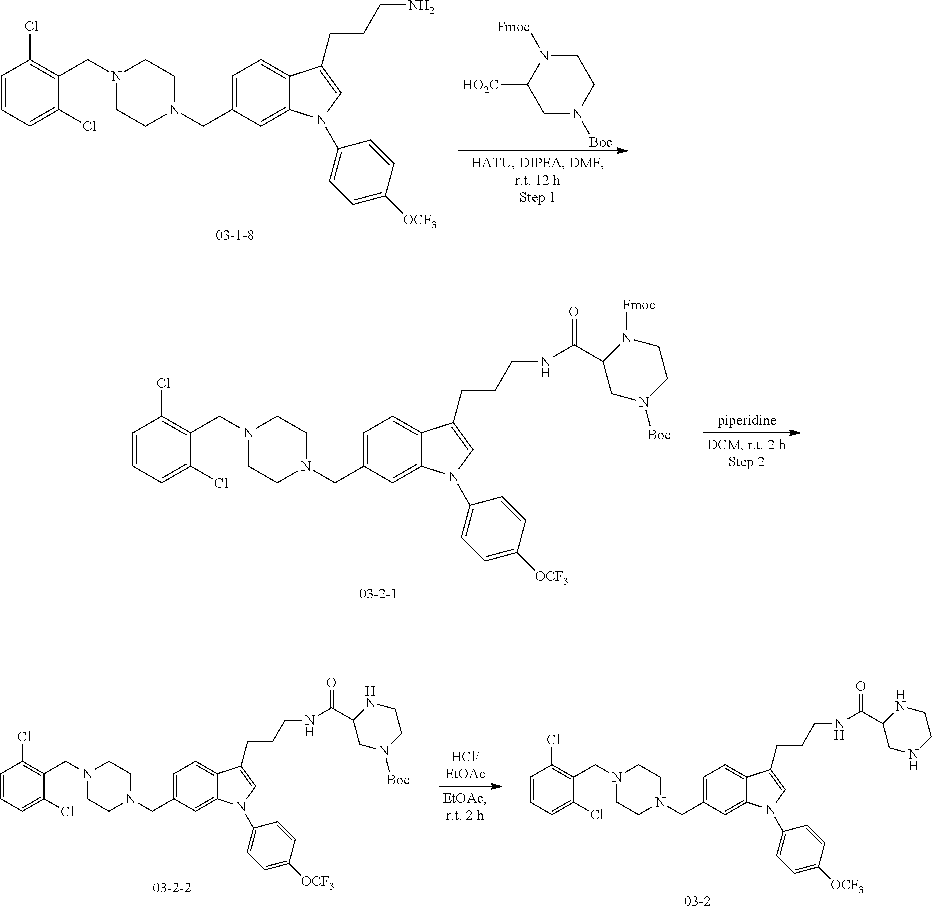

### Step 1: O4-tert-butyl O1-(9H-fluoren-9-ylmethyl) 2-[3-[6-[[4-[(2,6-dichlorophenyl)methyl]piperazin-1-yl]methyl]-1-[4-(trifluoromethoxy)phenyl]indol-3-yl]propylcarbamoyl]piperazine-1,4-dicarboxylate (03-2-1)

To a solution of 4-tert-butoxycarbonyl-1-(9H-fluoren-9-ylmethoxycarbonyl)piperazine-2-carboxylic acid (321.3 mg, 710.1 μmol, 1.2 eq) in DMF (5 mL) was added DIPEA (229.4 mg, 1.78 mmol, 310.0 μL, 3.0 eq) and HATU (450.0 mg, 1.18 mmol, 2.0 eq). The reaction was stirred at room temperature for 1 h. Then, compound 03-1-8 (350.0 mg, 591.7 μmol, 1.0 eq) was added and the resulting mixture was stirred at room temperature for 11 h, diluted with H₂O (20 mL) and filtered. The collected solid was purified by column (SiO₂) to give compound 03-2-1 (300.0 mg, 36%). M+H⁺=1025.3 (LCMS).

### Step 2: tert-butyl 4-amino-4-[3-[6-[[4-[(2,6-dichlorophenyl)methyl]piperazin-1-yl]methyl]-1-[4-(trifluoromethoxy)phenyl]indol-3-yl]propylcarbamoyl]piperidine-1-carboxylate (03-2-2)

To a solution of compound 03-2-1 (300.0 mg, 1.0 eq) in DCM (5 mL) was added piperidine (249.0 mg, 2.92 mmol, 289.5 μL, 10.0 eq). The reaction was stirred at room temperature for 2 h, poured into H₂O (10 mL) and evaporated under reduced pressure to give a residue. The residue was dissolved in MeOH (20 mL) and insoluble impurity was filtered. The filtrate was concentrated to give a crude product, which was purified by prep-HPLC to give compound 03-2-2 (200.0 mg, 75%, TFA salt). M+H⁺=803.3 (LCMS).

### Step 3: tert-butyl 3-[3-[6-[[4-[(2,6-dichlorophenyl)methyl]piperazin-1-yl]methyl]-1-[4-(trifluoromethoxy)phenyl]indol-3-yl]propylcarbamoyl]piperazine (03-2)

Compound 03-2 was synthesized according to the procedure as described in step 9 in the synthesis of 02-1. M+H⁺=703.3 (LCMS). ¹H NMR (MeOD, 400 MHz, HCl): δ 7.80 (s, 2H), 7.75-7.71 (m, 2H), 7.53-7.48 (m, 6H), 7.42-7.37 (m, 1H), 7.31 (d, J=8.2 Hz, 1H), 4.54-4.44 (m, 3H), 4.32 (br s, 2H), 3.70 (br t, J=12.8 Hz, 2H), 3.59-3.31 (m, 13H), 2.90 (t, J=7.6 Hz, 3H), 2.07-1.95 (m, 3H).

### Example 7: Synthesis of 3-[6-[[4-[(2,6-dichlorophenyl)methyl]piperazin-1-yl]methyl]-1-[4-(trifluoro methoxy)phenyl]indol-3-yl]cyclopentanamine (03-4)

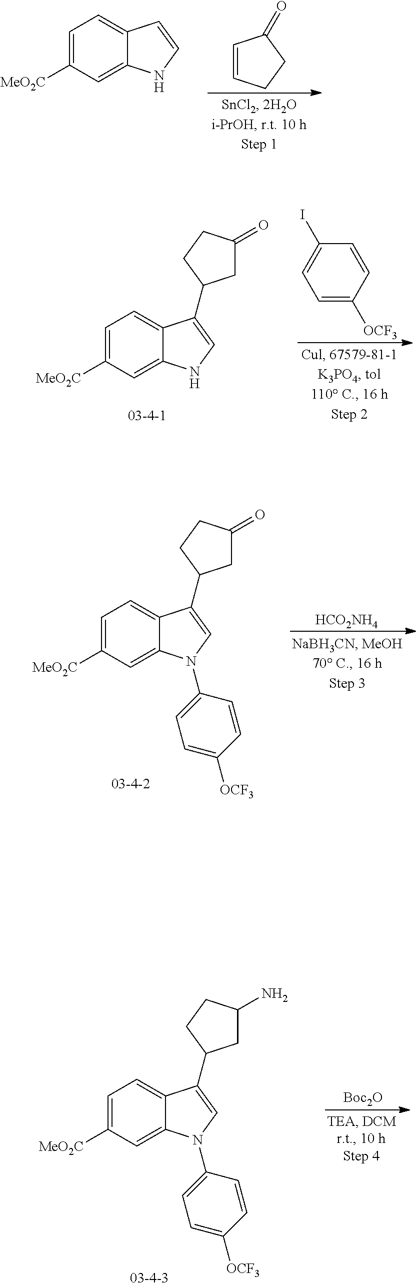

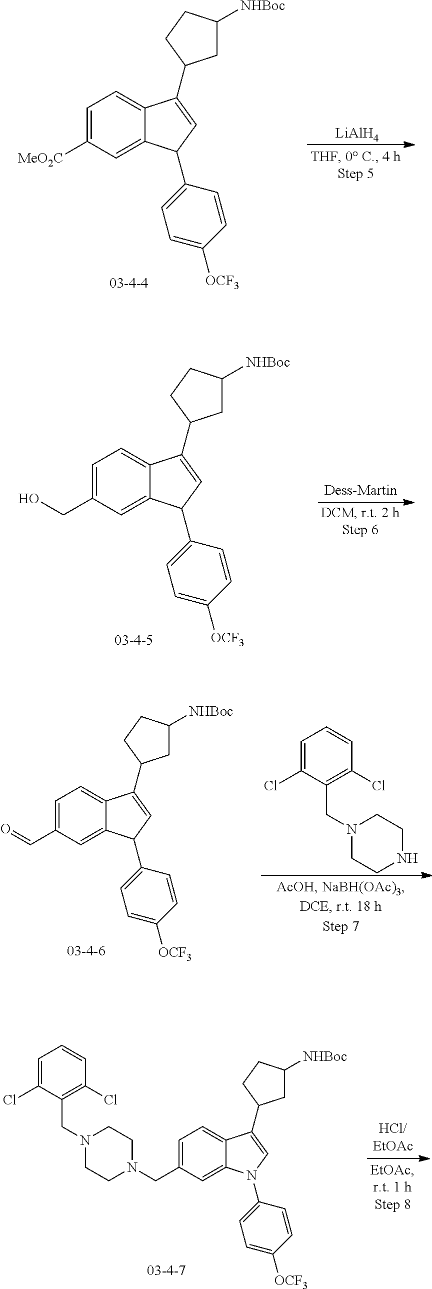 [SMILES: COC(=O)c1ccc2c(c1)C(c1ccc(OC(F)(F)F)cc1)C=C2C1CCC(NC(=O)OC(C)(C)C)C1 ; "LiAlH4" ; CC(C)(C)OC(=O)NC1CCC(C2=CC(c3ccc(OC(F)(F)F)cc3)c3cc(CO)ccc32)C1 ; CC(C)(C)OC(=O)NC1CCC(C2=CC(c3ccc(OC(F)(F)F)cc3)c3cc(C=O)ccc32)C1 ; Clc1cccc(Cl)c1CN1CCNCC1 ; CC(C)(C)OC(=O)NC1CCC(c2cn(-c3ccc(OC(F)(F)F)cc3)c3cc(CN4CCN(Cc5c(Cl)cccc5Cl)CC4)ccc23)C1]

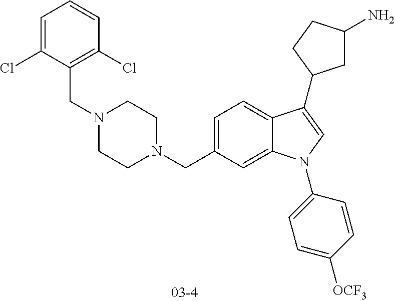 [SMILES: NC1CCC(c2cn(-c3ccc(OC(F)(F)F)cc3)c3cc(CN4CCN(Cc5c(Cl)cccc5Cl)CC4)ccc23)C1]

### Step 1: Methyl 3-(3-oxocyclopentyl)-1H-indole-6-carboxylate (03-4-1)

To a solution of methyl 1H-indole-6-carboxylate (17.5 g, 99.9 mmol, 1.0 eq) in i-PrOH (200 mL) was added cyclopent-2-en-1-one (16.4 g, 199.8 mmol, 16.7 mL, 2.0 eq) and SnCl₂*2H₂O (2.25 g, 9.99 mmol, 831.8 μL, 0.1 eq) under N₂. After addition, the mixture was stirred at room temperature for 16 h. The reaction mixture was concentrated and diluted with DCM (250 mL). The organic layer was separated and washed with H₂O (200 mL), brine (200 mL), dried over anhydrous Na₂SO₄, filtered, and concentrated under reduced pressure to give a residue. The residue was purified by column chromatography (SiO₂) to give compound 03-4-1 (22.4 g, 79% yield). ¹HNMR (CDCl₃, 400 MHz): δ 8.38 (s, 1H) 8.17 (s, 1H) 7.84 (d, J=8.8 Hz, 1H) 7.66 (d, J=8.8 Hz, 1H) 7.19 (d, J=2.01 Hz, 1H) 3.97 (s, 3H) 3.74-3.77 (m, 1H) 2.77-2.83 (m, 1H) 2.58-2.59 (m, 1H) 2.39-2.48 (m, 3H) 2.15-2.17 (m, 1H).

### Step 2: 3-(3-oxocyclopentyl)-1-[4-(trifluoromethoxy)phenyl]indole-6-carboxylate (03-4-2)

To a solution of compound 03-4-1 (5.30 g, 20.6 mmol, 1.0 eq) in toluene (150 mL) was added 1-iodo-4-(trifluoromethoxy)benzene (7.12 g, 24.7 mmol, 3.87 mL, 1.2 eq), K₃PO₄ (10.9 g, 51.5 mmol, 2.5 eq), (1S,2S)—N1,N2-dimethylcyclohexane-1,2-diamine (879.0 mg, 6.18 mmol, 0.3 eq) and CuI (392.3 mg, 2.06 mmol, 0.1 eq) under N₂ protection. The mixture was stirred at 110° C. for 18 h, cooled to room temperature, quenched by addition of H₂O (250 mL) at room temperature, and diluted with EtOAc (250 mL). The organic layer was separated, washed with brine (300 mL), dried over anhydrous Na₂SO₄, filtered and concentrated under reduced pressure to give a residue. The residue was purified by column chromatography (SiO₂) to give compound 03-4-2 (7.00 g, 81% yield).

### Step 3: methyl 3-(3-aminocyclopentyl)-1-[4-(trifluoromethoxy)phenyl]indole-6-carboxylate (03-4-3)

To a solution of compound 03-4-2 (7.00 g, 1.0 eq) in MeOH (150 mL) was added ammonium formate (10.6 g, 167.7 mmol, 10.0 eq) and NaBH₃CN (3.16 g, 50.3 mmol, 3.0 eq). The mixture was stirred at 70° C. for 18 h. The reaction was concentrated directly to remove the solvent and the residue was dissolved in DCM (150 mL). The mixture was stirred at room temperature for 0.5 h and then filtered. The filtrate was concentrated to give compound 03-4-3 (7.00 g, crude, 8.71 mmol, 52% yield) which was used directly in next step.

### Step 4: methyl 3-[3-(tert-butoxycarbonylamino)cyclopentyl]-1-[4-(trifluoromethoxy)phenyl]indole-6-carboxylate (03-4-4)

To a solution of the compound 03-4-3 (3.60 g, 8.60 mmol, 1.0 eq) in MeOH (150 mL) was added (Boc)₂O (2.82 g, 12.9 mmol, 1.5 eq) and TEA (2.61 g, 25.8 mmol, 3.0 eq). The mixture was stirred at room temperature for 4 h. The reaction mixture was concentrated to remove MeOH and then diluted with DCM (150 mL). The mixture was washed with H₂O (100 mL*2), brine (100 mL), dried over anhydrous Na₂SO₄, filtered and concentrated under reduced pressure. The residue was purified by column chromatography (SiO₂) to give compound 03-4-4 (3.00 g, 5.27 mmol, 61% yield). ¹H NMR (CDCl₃, 400 MHz) δ 8.11 (s, 1H), 7.77-7.79 (d, 1H), 7.61-7.62 (d, 1H), 7.42-7.45 (m, 2H) 7.33 (m, 2H) 7.15-7.18 (d, 2H) 4.52 (m, 1H) 4.05-4.09 (m, 1H) 3.85 (s, 3H) 3.29-3.45 (m, 1H) 2.58-2.59 (m, 1H) 2.12-2.25 (m, 2H) 1.49-1.88 (m, 3H) 1.39 (s, 9H).

### Step 5: tert-butyl N-[3-[6-(hydroxymethyl)-1-[4-(trifluoromethoxy)phenyl]indol-3-yl]cyclopentyl]carbamate (03-4-5)

To a solution of compound 03-4-4 (1.00 g, 1.93 mmol, 1.0 eq) in THF (10 mL) was added LAH (197.8 mg, 5.21 mmol, 2.7 eq) portion-wise slowly at 0° C. After addition, the mixture was stirred at 0° C. for 2 h. The reaction mixture was quenched by addition of H₂O (0.2 mL) at 0° C., followed by aqueous NaOH (1 N, 0.2 mL), and then diluted with DCM (30 mL). The reaction mixture was filtered and the filtrate was concentrated under reduced pressure to give a residue. The residue was purified by column chromatography (SiO₂) to give compound 03-4-5 (520.0 mg, 817.1 μmol, 42% yield). ¹H NMR (CDCl₃, 400 MHz) δ 7.64 (d, 1H) 7.48-7.51 (m, 3H) 7.35-7.37 (m, 2H) 7.16 (d, 1H) 7.08 (d, 1H) 4.77-4.79 (d, J=5.73 Hz, 2H) 4.59-4.62 (m, 1H) 4.06-4.14 (m, 1H) 3.35-3.50 (m, 1H) 2.58-2.71 (m, 1H) 2.04-2.30 (m, 3H) 1.63-1.66 (m, 1H) 1.56-1.61 (m, 3H) 1.46 (s, 9H).

Compound 03-4 was synthesized from compound 03-4-5 following the procedures described in Steps 4-6 for the synthesis of compound 02-5.

### Step 6: tert-Butyl N-[3-[6-formyl-1-[4-(trifluoromethoxy)phenyl]indol-3-yl]cyclopentyl]carbamate (03-4-6)

Compound 03-1-6 was sythesized from compound 03-1-5 following the procedure described for the sythesis of compound 02-5-4. ¹H NMR (CDCl₃, 400 MHz): δ 9.99 (s, 1H) 8.03-8.10 (m, 1H) 7.83-7.90 (m, 1H) 7.67-7.75 (m, 2H) 7.58-7.66 (m, 2H) 7.49-7.57 (m, 2H) 4.07-4.14 (m, 2H) 3.43-3.60 (m, 1H) 2.15-2.25 (m, 1H) 2.01-2.13 (m, 3H) 1.65-1.97 (m, 3H) 1.48 (s, 9H)

### Step 8: 3-[6-[[4-[(2, 6-dichlorophenyl)methyl]piperazin-1-yl]methyl]-1-[4-(trifluoromethoxy)phenyl]indol-3-yl]cyclopentanamine (03-4)

Compound 03-4 was sythesized from compound 03-4-7 following the procedure described for the sythesis of compound 02-5. ¹H NMR (MeOD, 400 MHz, HCl): δ 7.82-7.85 (m, 2H) 7.74-7.76 (m, 2H) 7.53 (br d, J=7.53 Hz, 5H) 7.44-7.46 (m, 1H) 7.38 (s, 1H) 4.54 (s, 2H) 4.44 (s, 2H) 3.83-3.91 (m, 1H) 3.71-3.73 (m, 1H) 3.51 (s, 9H) 2.73-2.76 (m, 1H) 2.31-2.42 (m, 3H) 1.84-1.94 (m, 2H).

### Example 8: Synthesis of 4-Amino-N-[3-[5-[[2-[(2,6-dichlorophenyl)methyl]-2,5-diaza bicyclo[2.2.1]heptan-5-yl]methyl]-3-[4-(trifluoromethoxy)phenyl]indol-1-yl]propyl]piperidine-4-carboxamide (04-1)

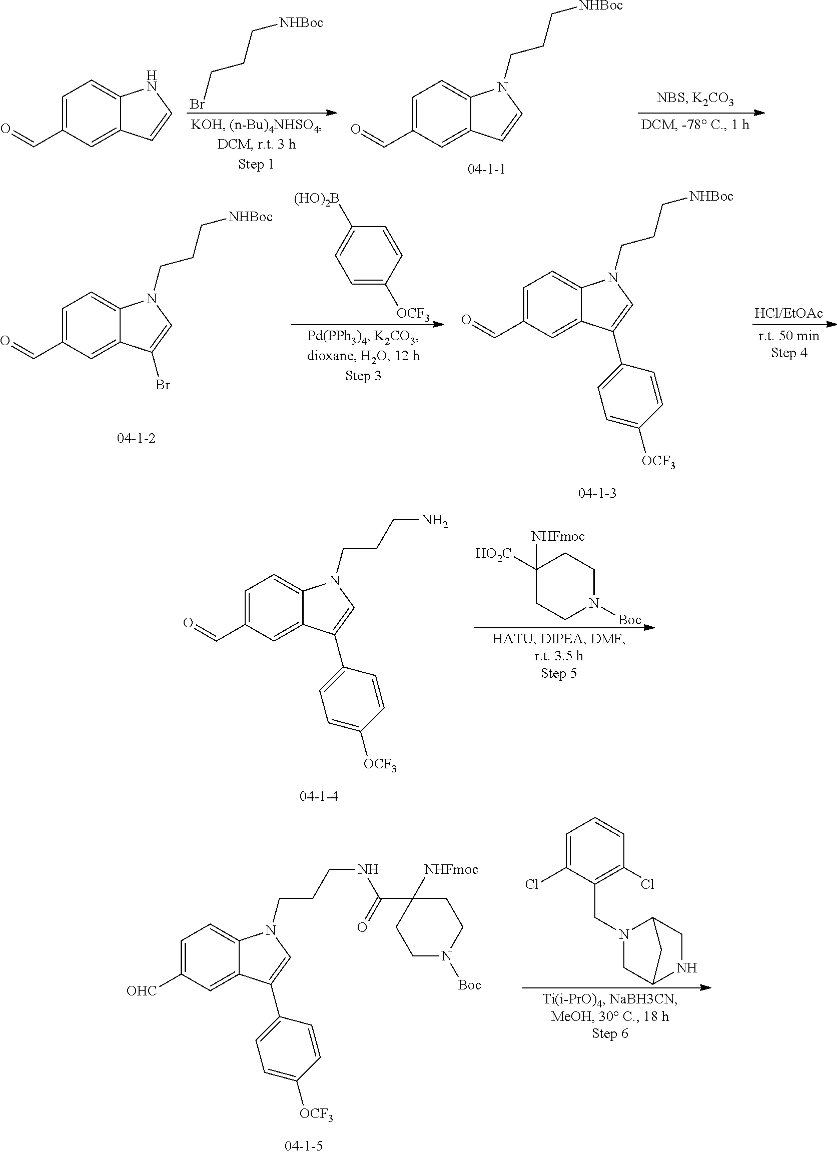

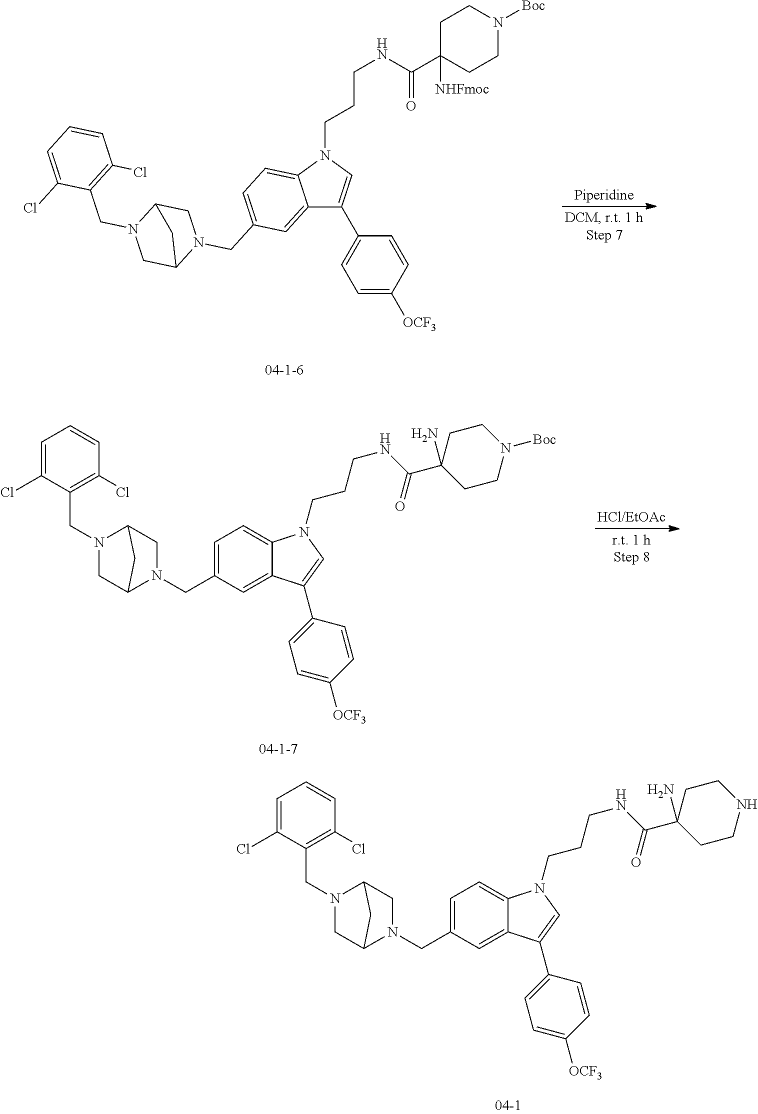

### Step 1:tert-butyl N-[3-(5-formylindol-1-yl)propyl]carbamate (04-1-1)

To a solution of 1H-indole-5-carbaldehyde (5.00 g, 34.4 mmol, 1.0 eq) in DCM (50 mL) was added KOH (4.83 g, 86.1 mmol, 2.5 eq) and tetrabutylammonium hydrosulfate (11.7 g, 34.4 mmol, 1.0 eq). The mixture was stirred at room temperature for 30 min. Then the tert-butyl N-(3-bromopropyl)carbamate (9.84 g, 41.3 mmol, 1.2 eq) was added. The resulting mixture was stirred at room temperature for 2 h. The reaction mixture was poured into NH₄Cl (sat.) 100 mL and extracted with DCM (200 mL*3). The combined organic layers were washed with brine (200 mL*2), dried over anhydrous Na₂SO₄, filtered and concentrated under reduced pressure to give a residue. The residue was purified by column chromatography (SiO₂) to give compound 04-1-1 (9.00 g, 29.8 mmol, 86% yield). M+H⁺=303.1 (LCMS). ¹H NMR (CDCl₃, 400 MHz): δ 9.92 (s, 1H), 8.05 (d, J=1.00 Hz, 1H), 7.68 (dd, J=8.60, 1.44 Hz, 1H), 7.31 (d, J=8.66 Hz, 1H), 7.15 (d, J=3.01 Hz, 1H), 6.57 (d, J=3.01 Hz, 1H), 4.72-4.63 (m, 1H), 4.12 (t, J=6.96 Hz, 2H), 3.04 (br s, 2H), 1.95 (t, J=6.84 Hz, 2H), 1.35 (s, 9H).

### Step 2: tert-butyl N-[3-(3-bromo-5-formyl-indol-1-yl)propyl]carbamate (04-1-2)

To a solution of tert-butyl N-[3-(5-formylindol-1-yl)propyl]carbamate (10.0 g, 33.1 mmol, 1.0 eq) in DCM (100 mL) was added NBS (6.47 g, 36.4 mmol, 1.1 eq) and K₂CO₃ (6.86 g, 49.6 mmol, 1.5 eq) at −78° C. The mixture was stirred at −78° C. for 1 h. The reaction mixture was diluted with H₂O 200 mL and extracted with DCM (200 mL*3). The combined organic layers were washed with brine (200 mL*3), dried over anhydrous Na₂SO₄, filtered and concentrated under reduced pressure to give a residue. The residue was purified by column chromatography (SiO₂) to give compound 04-1-2 (11.5 g, 30.2 mmol, 91% yield). M+H⁺=325.0 (LCMS). ¹H NMR (CDCl₃, 400 MHz): δ 10.07 (s, 1H), 8.09 (s, 1H), 7.86-7.81 (m, 1H), 7.41 (d, J=8.82 Hz, 1H), 7.29 (s, 1H), 4.65-4.53 (m, 1H), 4.21 (t, J=7.06 Hz, 2H), 3.16 (d, J=6.17 Hz, 2H), 2.05 (s, 2H), 1.45 (s, 9H).

### Step 3: tert-butyl N-[3-[5-formyl-3-[4-(trifluoromethoxy)phenyl]indol-1-yl]propyl]carbamate (04-1-3)

To a solution of tert-butyl N-[3-(3-bromo-5-formyl-indol-1-yl)propyl]carbamate (10.5 g, 27.5 mmol, 1.0 eq) in dioxane (100 mL) was added [4-(trifluoromethoxy)phenyl]boronic acid (8.51 g, 41.3 mmol, 1.5 eq), Pd(PPh₃)₄(1.59 g, 1.38 mmol, 0.05 eq) and the solution of K₂CO₃ (7.61 g, 55.1 mmol, 2.0 eq) in H₂O (10 mL). The suspension was degassed under vacuum and purged with N₂ several times. The mixture was stirred at 80° C. for 4 h. The reaction mixture was diluted with H₂O (100 mL) and extracted with EtOAc (200 mL*3). The combined organic layers were washed with brine (200 mL*2), dried over anhydrous Na₂SO₄, filtered and concentrated under reduced pressure to give a residue. The residue was purified by column chromatography (SiO₂) to give compound 04-1-3 (12.2 g, 19.8 mmol, 72% yield). M+H⁺=463.1 (LCMS). ¹H NMR (CDCl₃, 400 MHz): δ 10.07 (s, 1H), 8.38-8.41 (m, 1H), 7.88-7.84 (m, 1H), 7.68 (d, J=8.66 Hz, 2H), 7.51-7.47 (m, 1H), 7.45-7.42 (m, 1H), 7.37-7.32 (m, 2H), 4.73-4.66 (m, 1H), 4.30 (t, J=6.96 Hz, 2H), 3.27-3.18 (m, 2H), 2.17-2.09 (m, 2H), 1.47 (s, 9H).

### Step 4: 1-(3-aminopropyl)-3-[4-(trifluoromethoxy)phenyl]indole-5-carbaldehyde (04-1-4)

A solution of tert-butyl N-[3-[5-formyl-3-[4-(trifluoromethoxy)phenyl]indol-1-yl]propyl]carbamate (16.0 g, 34.6 mmol, 1.0 eq) in HCl/EtOAc (4 M, 120 mL, 13.9 eq) was stirred at room temperature for 50 min under N₂. The reaction mixture was concentrated in vacuum to give a residue which was washed by MTBE to give compound 04-1-4 (14.5 g, crude, HCl salt). M+H⁺=363.3 (LCMS).

### Step 5: tert-butyl 4-(9H-fluoren-9-ylmethoxycarbonylamino)-4-[3-[5-formyl-3-[4-(trifluoromethoxy)phenyl]indol-1-yl]propylcarbamoyl]piperidine-1-carboxylate (04-1-5)

A mixture of 1-tert-butoxycarbonyl-4-(9H-fluoren-9-ylmethoxycarbonylamino) piperidine-4-carboxylic acid (18.3 g, 39.1 mmol, 1.2 eq), HATU (14.9 g, 39.1 mmol, 1.2 eq) and DIPEA (16.9 g, 130.4 mmol, 22.8 mL, 4.0 eq) in DMF (150 mL) was degassed and stirred at room temperature for 0.5 h under N₂. Then 1-(3-aminopropyl)-3-[4-(trifluoro methoxy)phenyl]indole-5-carbaldehyde (13.0 g, 32.6 mmol, 1.0 eq, HCl salt) was added portionwise. The resulting mixture was degassed and stirred at room temperature for another 3 h under N₂, then poured into H₂O (120 mL). The mixture was extracted with EtOAc (40 mL*3). The organic phase was washed with brine (80 mL), dried over anhydrous Na₂SO₄, filtered and the filtrate was concentrated in vacuum to give a residue. The residue was purified by column chromatography (SiO₂) to give compound 04-1-5 (16.5 g, 18.2 mmol, 56% yield). M+H⁺=711.3 (LCMS). ¹H NMR (CDCl₃, 400 MHz): δ 9.86 (s, 1H), 8.21 (s, 1H), 7.65-7.54 (m, 3H), 7.48 (d, J=8.38 Hz, 1H), 7.40 (d, J=7.50 Hz, 2H), 7.32 (d, J=8.38 Hz, 1H), 7.28-7.18 (m, 3H), 7.16-7.06 (m, 4H), 4.33 (d, J=5.29 Hz, 1H), 4.09-3.99 (m, 2H), 3.69 (br s, 2H), 3.48 (dd, J=10.58, 6.62 Hz, 1H), 3.15 (br s, 2H), 2.99-2.82 (m, 3H), 1.96-1.71 (m, 6H), 1.33 (s, 9H).

### Step 6:tert-butyl 4-[3-[5-[[2-[(2,6-dichlorophenyl)methyl]-2,5-diazabicyclo[2.2.1]heptan-5-yl]methyl]-3-[4-(trifluoromethoxy)phenyl]indol-1-yl]propylcarbamoyl]-4-(9H-fluoren-9-ylmethoxycarbonylamino)piperidine-1-carboxylate (04-1-6)

A solution of 2-[(2,6-dichlorophenyl)methyl]-2,5-diazabicyclo[2.2.1]heptane (90.0 mg, 345.0 μmol, 1.0 eq), tert-butyl 4-(9H-fluoren-9-ylmethoxycarbonylamino)-4-[3-[5-formyl-3-[4-(trifluoromethoxy)phenyl]indol-1-yl]propylcarbamoyl]piperidine-1-carboxylate (340.5 mg, 420.0 μmol, 1.2 eq) and Ti(i-PrO)₄ (99.5 mg, 350.0 μmol, 103.6 μL, 1.0 eq) in MeOH (3 mL). The solution was degassed and stirred at 30° C. for 10 h under N₂. Then NaBH₃CN (65.9 mg, 1.05 mmol, 3.0 eq) was added portionwise. The resulting mixture was degassed and stirred at 30° C. for another 8 h under N₂. The reaction mixture was poured into H₂O (50 mL) and extracted with EtOAc (20 mL*3). The combined organic layers were washed with brine (30 mL), dried over anhydrous Na₂SO₄, filtered and concentrated in vacuum to give compound 04-1-6 (500.0 mg, crude). M+H⁺=1051.3 (LCMS).

### Step 7: tert-butyl 4-amino-4-[3-[5-[[2-[(2,6-dichlorophenyl)methyl]-2,5-diazabicyclo[2.2.1]heptan-5-yl]methyl]-3-[4-(trifluoromethoxy)phenyl]indol-1-yl]propylcarbamoyl]piperidine-1-carboxylate (04-1-7)

To a solution of compound 04-1-6 (500.0 mg, 475.3 μmol, 1.0 eq) in DCM (6 mL) was added piperidine (430.0 mg, 5.05 mmol, 500 μL, 10.6 eq) portionwise. The solution was degassed and stirred at room temperature for 1 h under N₂. The reaction mixture was concentrated under reduced pressure to remove DCM. Then H₂O (30 mL) was added and the mixture was concentrated under reduced pressure to remove piperidine and H₂O. Then MeOH (20 mL) was added and the mixture was concentrated to give a residue. The residue was purified by prep-HPLC (TFA condition) to give compound 04-1-7 (50.0 mg, 50.1 μmol, 11% yield, TFA salt). M+H⁺=829.3 (LCMS)¹H NMR (MeOD, 400 MHz): δ 8.08 (s, 1H), 7.77 (d, J=8.82 Hz, 2H), 7.70 (s, 1H), 7.60 (d, J=8.38 Hz, 1H), 7.38 (dd, J=18.08, 7.94 Hz, 5H), 7.30-7.25 (m, 1H), 4.55 (d, J=12.79 Hz, 1H), 4.42 (d, J=12.35 Hz, 1H), 4.32 (t, J=6.84 Hz, 2H), 4.24 (br s, 1H), 4.15 (d, J=13.23 Hz, 1H), 4.02 (d, J=12.79 Hz, 1H), 3.98-3.91 (m, 2H), 3.74 (br s, 1H), 3.59-3.46 (m, 2H), 3.24-3.06 (m, 5H), 2.25 (br s, 2H), 2.19-2.08 (m, 4H), 1.88-1.78 (m, 3H), 1.47 (s, 9H)

### Step 8: 4-amino-N-[3-[5-[[2-[(2,6-dichlorophenyl)methyl]-2,5-diazabicyclo[2.2.1]heptan-5-yl]methyl]-3-[4-(trifluoromethoxy)phenyl]indol-1-yl]propyl]piperidine-4-carboxamide (04-1)

A solution of intermediate 04-1-7 (90.0 mg, 95.4 μmol, 1.0 eq, TFA salt) in HCl/EtOAc (4 M, 5.00 mL, 209.7 eq) was degassed and stirred at room temperature for 1 h under N₂. The reaction mixture was filtered. The filter residue was washed with DCM (5 mL*3). The solid was collected and dried in vacuum to give 4-amino-N-[3-[5-[[2-[(2,6-dichlorophenyl)methyl]-2,5-diazabicyclo[2.2.1]heptan-5-yl]methyl]-3-[4-(trifluoromethoxy)phenyl]indol-1-yl]propyl]piperidine-4-carboxamide (44.6 mg, 55.7 μmol, 58% yield, HCl salt). M+H⁺=729.3 (LCMS). ¹H NMR (MeOD, 400 MHz): δ 8.21 (s, 1H), 7.87-7.78 (m, 3H), 7.66 (d, J=8.28 Hz, 1H), 7.52 (d, J=7.78 Hz, 3H), 7.46-7.40 (m, 1H), 7.35 (d, J=8.28 Hz, 2H), 4.68 (t, J=13.05 Hz, 2H), 4.61-4.48 (m, 3H), 4.38 (t, J=6.59 Hz, 3H), 3.93 (d, J=12.80 Hz, 1H), 3.64-3.50 (m, 2H), 3.48-3.41 (m, 2H), 3.40-3.33 (m, 5H), 2.79-2.64 (m, 3H), 2.56 (br s, 1H), 2.23-2.14 (m, 4H).

The following compounds were synthesized from intermediate 04-1-5 using procedures described in Steps 6-8 above for the preparation of compound 4-1.

| Comp | | | Mass | ¹H NMR (MeOD, |
|---|---|---|---|---|
| ID | Structure | Chemical Name | (M + H)⁺ | 400 MHz) |
| 04-17 | 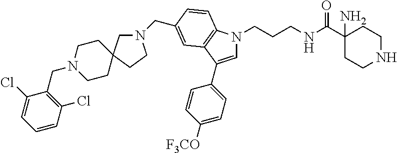 [SMILES: NC1(C(=O)NCCCn2cc(-c3ccc(OC(F)(F)F)cc3)c3cc(CN4CCC5(CCN(Cc6c(Cl)cccc6Cl)CC5)C4)ccc32)CCNCC1] | 4-amino-N-(3- (5-((8-(2,6- dichloro- benzyl)-2,8- diazaspiro[4.5] decan-2-yl) methyl)-3-(4- (trifluoro- methoxy) phenyl)-1H- indol-1-yl) propyl) piperidine-4- carboxamide | Calc'd for C₄₀H₄₈Cl₂F₃N₆O₂: 771.3; Found: 771.3 | δ 8.17 (br d, J = 15.9 Hz, 1H), 7.90-7.76 (m, 3H), 7.65 (br d, J = 7.1 Hz, 1H), 7.61-7.56 (m, 2H), 7.55-7.43 (m, 2H), 7.35 (br d, J = 7.9 Hz, 2H), 4.70 (br s, 2H), 4.57 (br d, J = 15.9 Hz, 2H), 4.38 (br t, J = 6.4 Hz, 2H), 3.62 |
| | | dihydro- | | (br s, 4H), 3.51- |
| | | chloride | | 3.40 (m, 4H), |
| | | | | 3.40-3.33 (m, |
| | | | | 4H), 3.20 |
| | | | | (br d, J = 11.5 |
| | | | | Hz, 1H), 2.72 |
| | | | | (br s, 2H), 2.40- |
| | | | | 2.10 (m, 7H), |
| | | | | 2.10-1.88 (m, |
| | | | | 4H) |
| 04-5 | 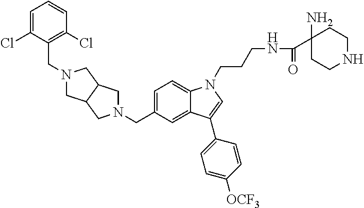 [SMILES: NC1(C(=O)NCCCn2cc(-c3ccc(OC(F)(F)F)cc3)c3cc(CN4CC5CN(Cc6c(Cl)cccc6Cl)CC5C4)ccc32)CCNCC1] | 4-amino-N-[3-[5- [[5-[(2,6- dichlorophenyl) methyl]-1,3,3a, 4,6,6a- hexahydropyrrolo [3,4-c]pyrrol-2- yl]methyl]-3-[4- (trifluoromethoxy) phenyl]indol-1- yl]propyl]piperi- dine-4- carboxamide,, hydrochloride salt | Calc'd for C₃₈H₄₄Cl₂F₃N₆O₂: 743.3; Found: 743.3 | δ 8.13 (s, 1H), 7.76-7.69 (m, 2H), 7.53 (s, 1H), 7.51-7.49 (m, 1H), 7.47-7.41 (m, 2H), 7.39- 7.37 (m, 2H), 7.30-7.23 (m, 2H), 4.47 (s, 2H), 4.28-4.25 (s, 2H), 3.38-3.34 (m, 14H), 3.28-3.23 (m, 3H), 2.64- 2.58 (m, 2H), 2.10-2.06 (m, 4H), 1.84 (s, 1H), 1.19 (s, 1H) |
| 04-9 | 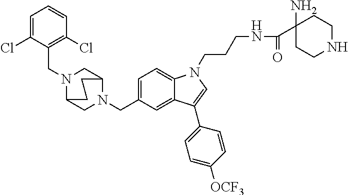 [SMILES: NC1(C(=O)NCCCn2cc(-c3ccc(OC(F)(F)F)cc3)c3cc(CN4CC5CCC4CN5Cc4c(Cl)cccc4Cl)ccc32)CCNCC1] | 4-amino-N-[3- [5-[[2-[(2,6- dichlorophenyl) methyl]-2,5- diazabicyclo [2.2.2]octan-5- yl]methyl]-3-[4- (trifluoromethoxy) phenyl]indol-1- yl]propyl]piperi- dine-4- carboxamide,, hydrochloride salt | Calc'd for C₃₈H₄₄Cl₂F₃N₆O₂: 743.3; Found: 743.3 | δ 8.21 (s, 1H), 7.68-7.66 (m, 3H), 7.61-7.53 (m, 1H), 7.51- 7.42 (m, 3H), 7.40-7.36 (m, 3H), 4.70 (s, 2H), 4.40-4.37 (m, 4H), 4.10-3.51 (m, 3H), 3.48- 3.38 (m, 9H), 2.76-2.72 (m, 2H), 2.21-2.18 (m, 8H) |
| 04-25 | 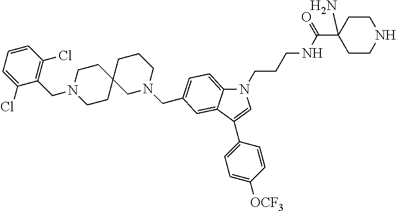 [SMILES: NC1(C(=O)NCCCn2cc(-c3ccc(OC(F)(F)F)cc3)c3cc(CN4CCCC5(CCN(Cc6c(Cl)cccc6Cl)CC5)C4)ccc32)CCNCC1] | 4-amino-N-(3- (5-((9-(2,6- dichlorobenzyl)- 2,9-diazaspiro [5.5]undecan-2- yl)methyl)-3-(4- (trifluoro- methoxy) phenyl)-1H- indol-1- yl)propyl)piperi- dine-4- carboxamide, formic acid salt | Calc'd for C₄₁H₅₀Cl₂F₃N₆O₂: 785.3; Found: 785.4 | δ 8.44 (brs, 2 H) 8.05 (s, 1 H) 7.80 (d, J = 8.8 Hz 2 H) 7.71 (s, 1 H) 7.63 (d, J = 8.8 Hz 1 H) 7.37-7.38 (m, 5 H) 7.24-7.28 (m, 1 H) 4.33-4.37 (m, 4 H) 3.78 (s, 2 H) 3.28-3.37 (m, 11 H) 2.52- 2.62 (m, 4 H) 2.13-2.25 (m, 4 H) 1.84 (m, 2 H) 1.58-1.65 (m, 7 H) |
| 04-27 | 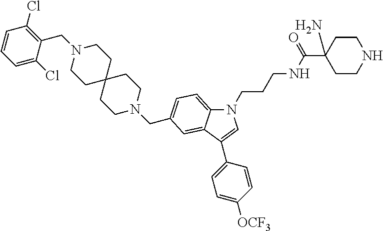 [SMILES: NC1(C(=O)NCCCn2cc(-c3ccc(OC(F)(F)F)cc3)c3cc(CN4CCC5(CC4)CCN(Cc4c(Cl)cccc4Cl)CC5)ccc32)CCNCC1] | 4-amino-N-(3- (5-((9-(2,6- dichlorobenzyl)- 3,9- diazaspiro[5.5] undecan-3- yl)methyl)-3-(4- (trifluoro- methoxy) phenyl)-1H- indol-1- yl)propyl)piperi- dine-4- carboxamide formic acid salt | Calc'd for C₄₁H₅₀Cl₂F₃N₆O₂: 785.3; Found: 785.4 | δ 8.48 (brs, 3 H) 8.06 (s, 1 H) 7.80 (d, J = 8.0 Hz 2 H) 7.71 (s, 1 H) 7.63 (d, J = 7.2 Hz 1 H) 7.36-7.42 (m, 5 H) 7.29-7.31 (m, 1 H) 4.41 (s, 2 H) 4.35 (s, 2 H) 3.89 (s, 2 H) 3.22-3.73 (m, 11 H) 2.69 (m, 4 H) 2.14- 2.23 (m, 4 H) 1.63-1.76 (m, 9 H) |
| 04-29 | 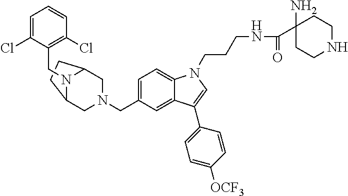 [SMILES: NC1(C(=O)NCCCn2cc(-c3ccc(OC(F)(F)F)cc3)c3cc(CN4CC5CCC(C4)N5Cc4c(Cl)cccc4Cl)ccc32)CCNCC1] | 4-amino-N-(3- (5-((8-(2,6- dichlorobenzyl)- 3,8- diazabicyclo [3.2.1]octan-3- yl)methyl)-3-(4- (trifluoro- methoxy) phenyl)-1H- indol-1- yl)propyl)piperi- dine-4- carboxamide, formic acid salt | Calc'd for C₃₈H₄₄Cl₂F₃N₆O₂: 743.3; Found: 743.3 | δ 8.53 (s, 1H), 7.83 (s, 1H), 7.75-7.72 (br d, J = 12 Hz, 2H), 7.56 (s, 1H), 7.46- 7.43 (m, 1H), 7.39-7.37 (m, 2H), 7.35-7.33 (m, 2H), 7.31- 7.26 (m, 2H), 4.30 (t, 2H), 3.83 (s, 2H), 3.70 (s, 2H), 3.68-3.33 (m, 2H), 3.28-3.25 (m, 6H), 2.71- 2.69 (m, 2H), |
| | | | | 2.50-2.48 (m, |
| | | | | 2H), 2.20-2.11 |
| | | | | (m, 6H), 2.10- |
| | | | | 2.95 (m, 2H), |
| | | | | 1.55-1.51 (m, |
| | | | | 2H) |
| 04-37 | 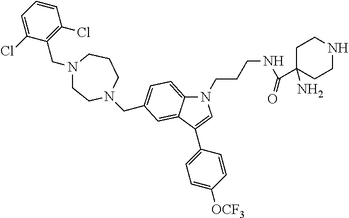 [SMILES: NC1(C(=O)NCCCn2cc(-c3ccc(OC(F)(F)F)cc3)c3cc(CN4CCCN(Cc5c(Cl)cccc5Cl)CC4)ccc32)CCNCC1] | 4-amino-N-(3- (5-((4-(2,6- dichlorobenzyl)- 1,4-diazepan-1- yl)methyl)-3-(4- (trifluoro- methoxy) phenyl)-1H- indol-1- yl)propyl)piperi- dine-4- carboxamide, hydrochloride salt | Calc'd for C₃₇H₄₄Cl₂F₃N₆O₂: 731.3; Found: 731.3 | δ 8.16 (s, 1H), 7.85-7.82 (m, 2H), 7.80 (s, 1H), 7.67 (d, J = 8.6 Hz, 1H), 7.58- 7.54 (m, 2H), 7.52-7.43 (m, 2H), 7.35 (d, J = 7.9 Hz, 2H), 4.80-4.63 (m, 2H), 4.59 (s, 2H), 4.38 (t, J = 6.8 Hz, 2H), 3.85 (br s, 1H), 3.89- 3.81 (m, 1H), 3.46-3.32 (m, 8H), 2.79-2.61 (m, 2H), 2.35 (br s, 2H), 2.25-2.10 (m, 5H) |

### Example 9: Synthesis of 4-amino-N-[3-[5-[[7-[(2,6-dichlorophenyl)methyl]-2,7-diaza spiro[3.5]nonan-2-yl]methyl]-3-[4-(trifluoromethoxy)phenyl]indol-1-yl]propyl]piperidine-4-carboxamide (04-21)

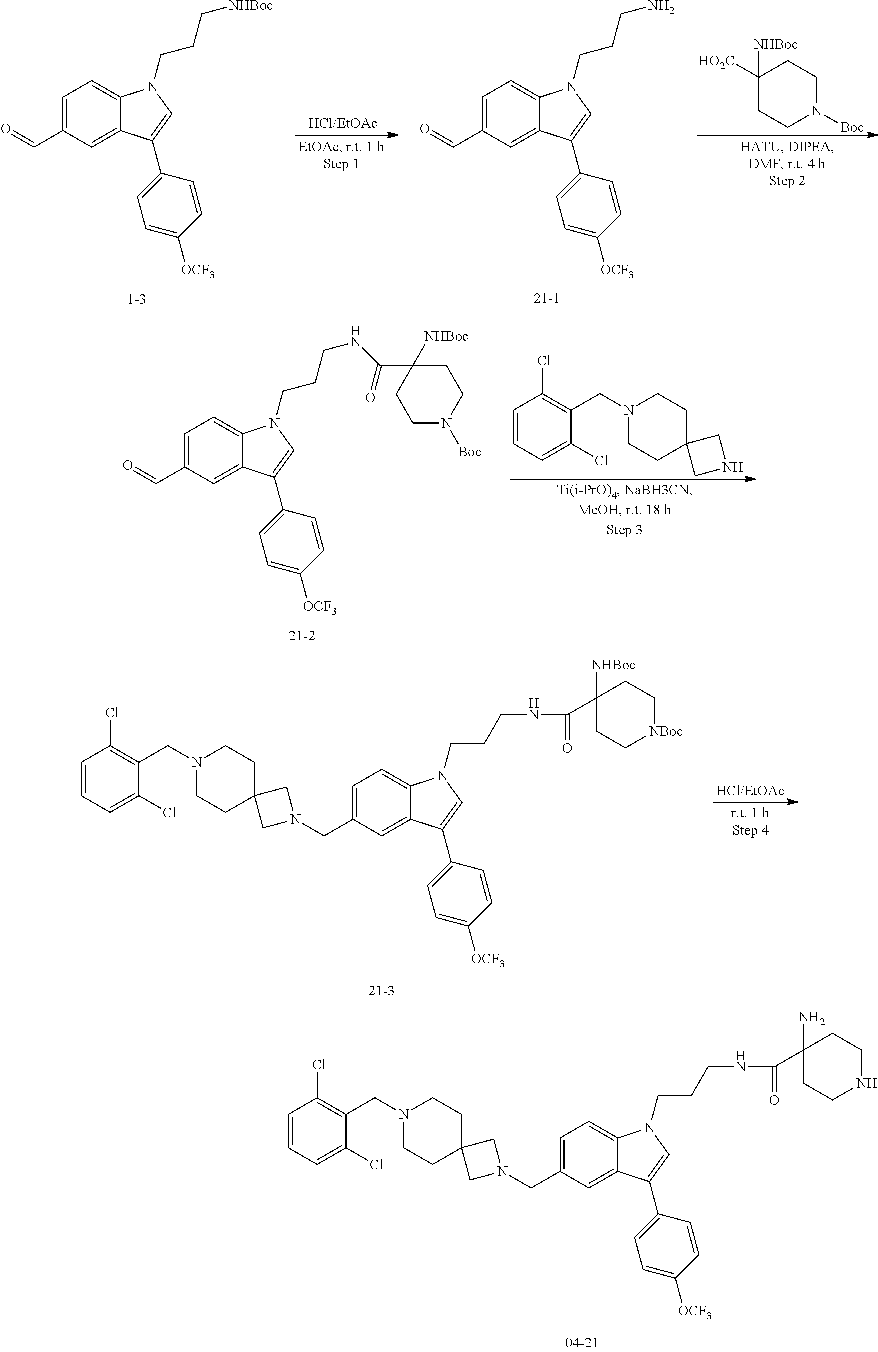

### Step 1: 1-(3-aminopropyl)-3-[4-(trifluoromethoxy)phenyl]indole-5-carbaldehyde (21-1)

To a solution of tert-butyl N-[3-[5-formyl-3-[4-(trifluoromethoxy)phenyl]indol-1-yl]propyl]carbamate (10.0 g, 21.6 mmol, 1.0 eq) in EtOAc (100 mL) was added HCl/EtOAc (4 M, 100.0 mL, 18.5 eq). The mixture was stirred at room temperature for 1 h. The reaction mixture was concentrated under reduced pressure to remove EtOAc. The residue was diluted with H₂O (200 mL) and adjusted pH to 9 with Na₂CO₃, then extracted with EtOAc (200 mL*3). The combined organic layers were washed with brine (200 mL*2), dried over anhydrous Na₂SO₄, filtered and concentrated under reduced pressure to give compound 21-1 (8.50 g, 7.46 mmol, 35% yield, HCl salt), which was used into the next step without further purification.

### Step 2: tert-butyl 4-(tert-butoxycarbonylamino)-4-[3-[5-formyl-3-[4-(trifluoromethoxy)phenyl]indol-1-yl]propylcarbamoyl]piperidine-1-carboxylate (21-2)

To a solution of 1-tert-butoxycarbonyl-4-(tert-butoxycarbonylamino)piperidine-4-carboxylic acid (3.04 g, 8.82 mmol, 1.1 eq) in DMF (100 mL) was added HATU (3.35 g, 8.82 mmol, 1.1 eq) and DIPEA (3.11 g, 24.1 mmol, 4.20 mL, 3.0 eq). The mixture was stirred at 0° C. for 0.5 h. Then the 1-(3-aminopropyl)-3-[4-(trifluoromethoxy)phenyl]indole-5-carbaldehyde (8.30 g, 8.02 mmol, 1.0 eq) was added to the reaction. The resulting mixture was stirred at 0° C. for more 2.5 h. The mixture was poured into H₂O (500 mL) and precipitation was formed. The mixture was filtered, and the cake was washed with H₂O (50 mL*2). The cake was dissolved into EtOAc (300 mL) and washed with brine (100 mL*2), dried with Na₂SO₄, filtered and concentrated under reduced pressure to give the residue. The residue was purified by column chromatography (SiO₂) to give compound 21-2 (3.00 g, 3.05 mmol, 38% yield).

### Step 3: tert-butyl 4-(tert-butoxycarbonylamino)-4-[3-[5-[[7-[(2,6-dichlorophenyl)methyl]-2,7-diazaspiro[3.5]nonan-2-yl]methyl]-3-[4-(trifluoromethoxy)phenyl]indol-1-yl]propylcarbamoyl]piperidine-1-carboxylate (21-3)

A mixture of 7-[(2,6-dichlorophenyl)methyl]-2,7-diazaspiro[3.5]nonane (50.0 mg, 155.4 μmol, 1.0 eq, HCl salt), tert-butyl 4-(tert-butoxycarbonylamino)-4-[3-[5-formyl-3-[4-(trifluoromethoxy)phenyl]indol-1-yl]propylcarbamoyl]piperidine-1-carboxylate (117.8 mg, 171.0 μmol, 1.1 eq), NaOAc (63.8 mg, 777.2 μmol, 5.0 eq) in DCE (2 mL) was degassed and stirred at room temperature for 6 hours under N₂. Then NaBH(OAc)₃ (98.8 mg, 466.3 μmol, 3.0 eq) was added portionwise. The whole mixture was degassed and stirred at room temperature for 12 h under N₂, then poured into H₂O (40 mL). The resulting mixture was extracted with EtOAc (15 mL*3). The combined organic layers were washed with brine (20 mL), dried over anhydrous Na₂SO₄, filtered and the filtrate was concentrated in vacuum to give a residue. The residue was purified by prep-TLC (SiO₂) to give compound 21-3 (50.0 mg, 51.2 μmol, 33% yield). ¹H NMR (CDCl₃, 400 MHz): δ 7.85 (s, 1H), 7.68 (br d, J=8.8 Hz, 2H), 7.40 (br d, J=7.9 Hz, 3H), 7.34-7.28 (m, 3H), 7.27 (d, J=2.2 Hz, 1H), 7.15-7.09 (m, 1H), 6.87 (br s, 1H), 4.80 (br s, 1H), 4.24 (br t, J=6.6 Hz, 2H), 4.02 (br s, 2H), 3.85 (br d, J=11.0 Hz, 2H), 3.65 (s, 2H), 3.41-3.25 (m, 5H), 3.07 (br t, J=11.2 Hz, 2H), 2.43 (br s, 4H), 2.13-1.89 (m, 7H), 1.80 (br s, 3H), 1.45 (d, J=11.0 Hz, 18H).

### Step 4: 4-amino-N-[3-[5-[[7-[(2,6-dichlorophenyl)methyl]-2,7-diazaspiro[3.5]nonan-2-yl]methyl]-3-[4-(trifluoromethoxy)phenyl]indol-1-yl]propyl]piperidine-4-carboxamide (4-21)

To a solution of tert-butyl 4-(tert-butoxycarbonylamino)-4-[3-[5-[[7-[(2,6-dichloro phenyl)methyl]-2,7-diazaspiro[3.5]nonan-2-yl]methyl]-3-[4-(trifluoromethoxy)phenyl]indol-1-yl]propylcarbamoyl]piperidine-1-carboxylate (50.0 mg, 1.0 eq) in EtOAc (1 mL) was added HCl/EtOAc (4 M, 1 mL, 76.6 eq). The mixture was stirred at room temperature for 1 h under N₂. The reaction mixture was filtered. The solid was washed with DCM (5 mL*3), collected and dried in vacuum to give compound 4-21 (21.5 mg, 27.0 μmol, 52% yield, HCl salt). M+H⁺=757.4 (LCMS). ¹H NMR (MeOD, 400 MHz): δ 8.12 (s, 1H), 7.83 (br d, J=8.8 Hz, 2H), 7.79 (s, 1H), 7.65 (br d, J=8.8 Hz, 1H), 7.62-7.56 (m, 2H), 7.55-7.49 (m, 1H), 7.44 (br d, J=8.4 Hz, 1H), 7.36 (br d, J=7.9 Hz, 2H), 4.67 (s, 2H), 4.57 (s, 2H), 4.37 (br t, J=6.8 Hz, 2H), 4.24-4.07 (m, 3H), 3.97 (br s, 1H), 3.60 (br s, 2H), 3.48-3.41 (m, 3H), 3.40-3.33 (m, 5H), 2.75-2.66 (m, 2H), 2.53-2.36 (m, 2H), 2.26-2.06 (m, 6H).

The following compounds were synthesized from intermediate 21-2 using procedures described in Steps 3 and 4 above for the preparation of compound 04-21.

| Comp | | | Mass | 1H NMR (MeOD, |
|---|---|---|---|---|
| ID | Structure | Chemical Name | (M + H⁺) | 400 MHz) |
| 04-31 | 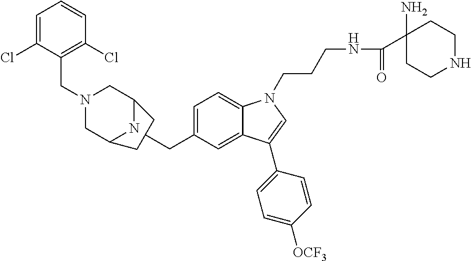 [SMILES: NC1(C(=O)NCCCn2cc(-c3ccc(OC(F)(F)F)cc3)c3cc(CC4CC5CN(Cc6c(Cl)cccc6Cl)CC(C4)N5)ccc32)CCNCC1] | 4-amino-N-(3-(5- ((3-(2,6- dichlorobenzyl)- 3,8- diazabicyclo[3.2.1] octan-8- yl)methyl)-3-(4- (trifluoromethoxy) phenyl)-1H- indol-1- yl)propyl) piperidine-4- carboxamide, hydrochloride salt | Calc'd for C₃₈H₄₄Cl₂F₃N₆O₂: 743.3; Found: 743.3 | δ 8.16 (s, 1H), 7.86- 7.77 (m, 3H), 7.66 (d, J = 8.5 Hz, 1H), 7.49- 7.38 (m, 3H), 7.37- 7.27 (m, 3H), 4.42- 4.30 (m, 4H), 4.02 (br s, 2H), 3.94 (br s, 2H), 3.53-3.42 (m, 2H), 3.42-3.32 (m, 4H), 3.07 (br s, 2H), 3.00 (br s, 2H), 2.79-2.67 (m, 2H), 2.35 (br s, 2H), 2.26-2.10 (m, 6H) |
| 04-11 | 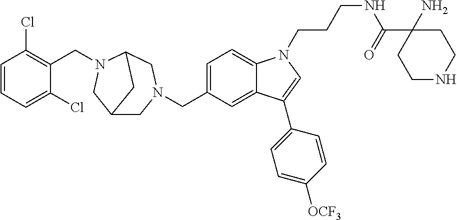 [SMILES: NC1(C(=O)NCCCn2cc(-c3ccc(OC(F)(F)F)cc3)c3cc(CN4CC5CC(C4)N(Cc4c(Cl)cccc4Cl)C5)ccc32)CCNCC1] | 4-amino-N-(3-(5- ((6-(2,6- dichlorobenzyl)- 3,6- diazabicyclo[3.2.1] octan-3- yl)methyl)-3-(4- (trifluoromethoxy) phenyl)-1H- indol-1- yl)propyl) piperidine-4- carboxamide, hydrochloride | Calc'd for C₃₈H₄₄Cl₂F₃N₆O₂: 743.3; Found: 743.2 | δ 8.25-8.16 (m, 1H), 7.84 (br d, J = 7.9 Hz, 2H), 7.78 (br s, 1H), 7.59 (br d, J = 7.9 Hz, 1H), 7.56-7.40 (m, 4H), 7.32 (br d, J = 8.4 Hz, 2H), 4.35 (br s, 6H), 3.70-3.41 (m, 6H), 3.33 (br s, 5H), 3.07-2.94 (m, 1H), 2.76-2.65 (m, 2H), 2.63-2.50 (m, 1H), 2.39-2.28 (m, 1H), 2.17 (br d, J = 6.6 Hz, 4H), 1.37-1.21 (m, 2H) |

### Example 10: Preparation of tert-butyl N-[3-[5-[[4-[(2,6-dichlorophenyl)methyl]piperazin-1-yl]methyl]-3-[4-(trifluoromethoxy)phenyl]indol-1-yl]propyl]carbamate (01-68)

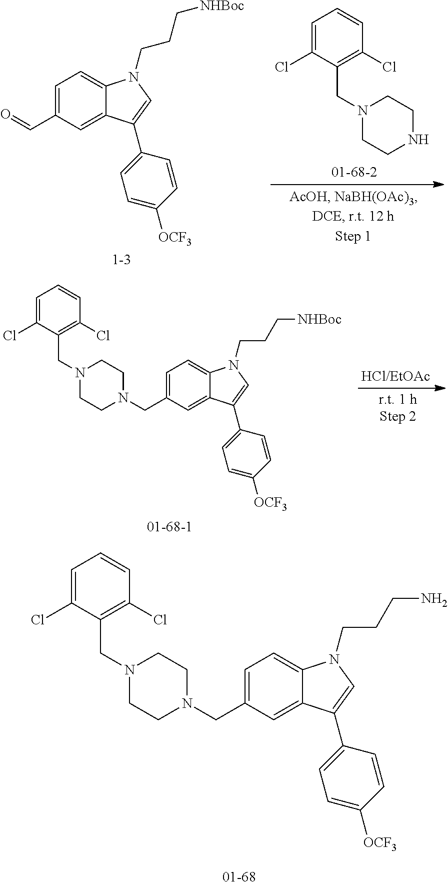 [SMILES: CC(C)(C)OC(=O)NCCCn1cc(-c2ccc(OC(F)(F)F)cc2)c2cc(C=O)ccc21 ; Clc1cccc(Cl)c1CN1CCNCC1 ; CC(C)(C)OC(=O)NCCCn1cc(-c2ccc(OC(F)(F)F)cc2)c2cc(CN3CCN(Cc4c(Cl)cccc4Cl)CC3)ccc21 ; NCCCn1cc(-c2ccc(OC(F)(F)F)cc2)c2cc(CN3CCN(Cc4c(Cl)cccc4Cl)CC3)ccc21]

### Step 1: tert-butyl N-[3-[5-[[4-[(2,6-dichlorophenyl)methyl]piperazin-1-yl]methyl]-3-[4-(trifluoromethoxy)phenyl]indol-1-yl]propyl]carbamate (01-68-1)

To a solution of compound 1-3 (11.2 g, 24.2 mmol, 1.0 eq) in DCE (100 mL) was added 1-[(2,6-dichlorophenyl)methyl]piperazine (7.13 g, 29.1 mmol, 1.2 eq), AcOH (1.45 g, 24.2 mmol, 1.39 mL, 1.0 eq) and NaBH(OAc)₃ (15.4 g, 72.7 mmol, 3.0 eq). The mixture was stirred at room temperature for 12 h, poured into saturated NaHCO₃ (100 mL) and extracted with DCM (100 mL*3). The combined organic layers were washed with brine (100 mL*2), dried over anhydrous Na₂SO₄, filtered and concentrated under reduced pressure to give the crude 01-68-1 (16.0 g, crude), which was used into the next step without further purification. M+H⁺=691.3 (LCMS).

### Step 2: 3-[5-[[4-[(2,6-dichlorophenyl)methyl]piperazin-1-yl]methyl]-3-[4-(trifluoromethoxy)phenyl]indol-1-yl]propan-1-amine (01-68)

To a solution of 01-68-1 (16.0 g, 23.1 mmol, 1.0 eq) in EtOAc (100 mL) was added HCl/EtOAc (4 M, 100.0 mL, 17.3 eq). The mixture was stirred at room temperature for 1 h, poured into H₂O (200 mL) and adjust pH to 8 with saturated NaHCO₃ solution. Then the product was extracted with EtOAc (200 mL*3). The combined organic layers were washed with brine (200 mL*2), dried over anhydrous Na₂SO₄, filtered and concentrated under reduced pressure to give the crude product 01-68 (12.2 g, 20.6 mmol, 89% yield). M+H⁺=591.2 (LCMS). ¹H NMR (CDCl₃, 400 MHz): δ 7.71 (s, 1H), 7.60 (d, J=8.38 Hz, 2H), 7.33-7.29 (m, 1H), 7.26-7.22 (m, 6H), 7.08 (d, J=7.94 Hz, 2H), 3.71 (s, 2H), 3.58 (s, 2H), 2.51 (d, J=4.85 Hz, 2H), 2.43 (br s, 2H), 1.97 (t, J=6.84 Hz, 2H), 1.33-1.25 (m, 2H).

### Preparation of Formic Acid Salt of 01-68:

To a solution of 01-68 (300.0 mg, 433.8 μmol, 1.0 eq) in DCM (3 mL) was added formic acid (1.22 g, 26.5 mmol, 1.0 mL, 61.1 eq). The mixture was stirred at room temperature for 12 h. The reaction mixture was concentrated under reduced pressure to remove DCM and the residue was lyophilized to give the crude product. The residue was purified by prep-HPLC (FA condition) to give compound 01-68 (150.0 mg, 235.0 μmol, 54% yield, FA salt). M+H⁺=591.3 (LCMS). ¹H NMR (MeOD, 400 MHz): δ 8.53 (s, 1H), 7.91 (s, 1H), 7.75 (d, J=7.3 Hz, 2H), 7.59 (s, 1H), 7.53 (d, J=8.0 Hz, 1H), 7.40-7.32 (m, 4H), 7.31-7.22 (m, 2H), 4.38 (t, J=6.7 Hz, 2H), 3.89 (s, 2H), 3.81 (s, 2H), 2.97-2.88 (m, 2H), 2.70 (br s, 8H), 2.27-2.18 (m, 2H).

### Example 11: Synthesis of 1-[(2,6-dichlorophenyl)methyl]piperazine (01-68-2)

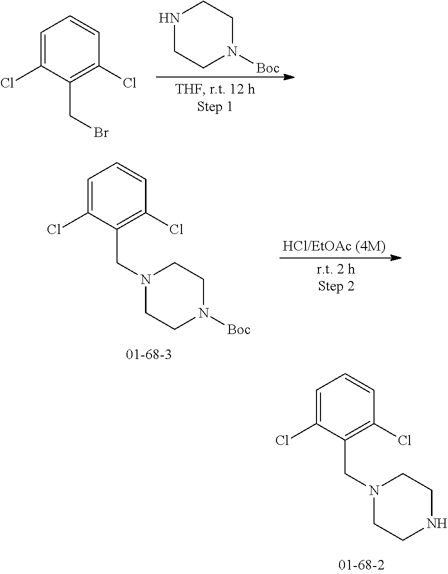

### Step 1: tert-butyl 4-[(2,6-dichlorophenyl)methyl]piperazine-1-carboxylate (01-68-3)

To a solution of tert-butyl piperazine-1-carboxylate (34.9 g, 187.6 mmol, 1.5 eq) in THF (135 mL) at 0° C., a solution of 2-(bromomethyl)-1,3-dichloro-benzene (30.0 g, 125.0 mmol, 1.0 eq) in THF (300 mL) was added dropwise over 10 min. The resulting mixture was slowly allowed to warm to room temperature and stirred for 12 h. The reaction mixture was concentrated under reduced pressure. The residue was diluted with H₂O (500 mL) and extracted with DCM (500 mL*3). The combined organic layers were washed with brine (500 mL*2), dried over anhydrous Na₂SO₄, filtered and concentrated under reduced pressure to give a residue. The residue was purified by column chromatography (SiO₂) to give compound 01-68-3 (31.0 g, 89.8 mmol, 72% yield). M+H⁺=345.1 (LCMS). ¹H NMR (CDCl₃, 400 MHz): δ 7.31 (s, 1H), 7.29 (s, 1H), 7.15 (d, J=8.16 Hz, 1H), 3.75 (s, 2H), 3.41-3.36 (m, 4H), 2.52 (d, J=4.27 Hz, 4H), 1.46 (s, 9H).

### Step 2: 1-[(2,6-dichlorophenyl)methyl]piperazine (01-68-2)

To a solution of 01-68-3 (30.0 g, 86.9 mmol, 1.00 eq) in EtOAc (150 mL) was added HCl/EtOAc (4 M, 150 mL, 6.9 eq). The mixture was stirred at room temperature for 2 h. The mixture was mixed with an earlier batch from 1 g of 01-68-3. The mixture was concentrated under reduced pressure to remove EtOAc. The residue was dissolved in H₂O (300 mL), adjust pH to 8 with NaHCO₃ and extracted with DCM (300 mL*6). The combined organic layers were washed with brine (500 mL*3) and dried over anhydrous Na₂SO₄, filtered and concentrated under reduced pressure to give compound 01-68-2 (20.0 g, 81.6 mmol, 94% yield). M+H⁺=245.1 (LCMS). ¹H NMR (CDCl₃, 400 MHz): δ 7.30 (d, J=7.94 Hz, 2H) 7.18-7.12 (m, 1H) 6.68 (br s, 1H) 3.78 (s, 2H) 3.05-2.99 (m, 4H) 2.75-2.70 (m, 4H).

### Example 12: Synthesis of 3-[5-[[4-[(2,6-dichlorophenyl)methyl]piperazin-1-yl]methyl]-3-[4-(trifluoromethoxy)phenyl]indol-1-yl]-N-methyl-propan-1-amine (01-7)

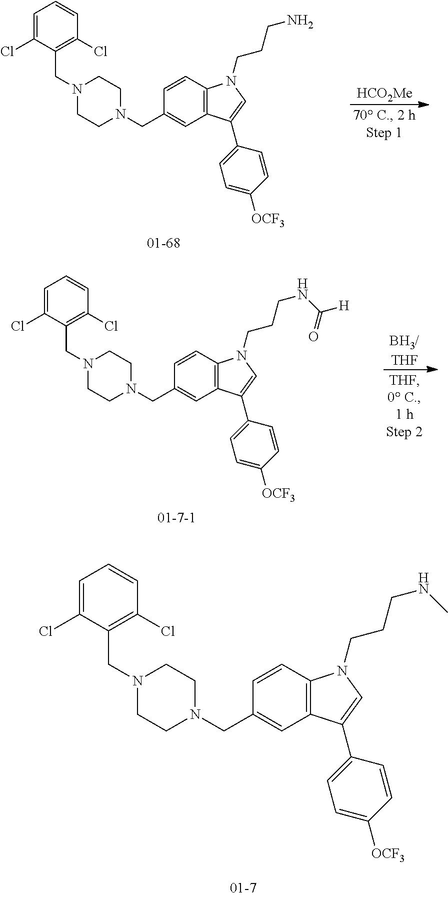 [SMILES: NCCCn1cc(-c2ccc(OC(F)(F)F)cc2)c2cc(CN3CCN(Cc4c(Cl)cccc4Cl)CC3)ccc21 ; "HCO2Me" ; O=CNCCCn1cc(-c2ccc(OC(F)(F)F)cc2)c2cc(CN3CCN(Cc4c(Cl)cccc4Cl)CC3)ccc21 ; "BH3/THF" ; CNCCCn1cc(-c2ccc(OC(F)(F)F)cc2)c2cc(CN3CCN(Cc4c(Cl)cccc4Cl)CC3)ccc21]

### Step 1: N-[3-[5-[[4-[(2,6-dichlorophenyl)methyl]piperazin-1-yl]methyl]-3-[4-(trifluoromethoxy)phenyl]indol-1-yl]propyl]formamide (01-7-1)

A solution of 01-68 (200.0 mg, 338.1 μmol, 1.0 eq) in HCO₂Me (8 mL) was degassed and purged with N₂ for 3 times, and then the mixture was stirred at 70° C. for 2 h under N₂ atmosphere. The reaction mixture was diluted with aqueous of NaHCO₃ (1 M) and extracted with EtOAc (3*10 mL). The combined organic layers were washed with brine (20 mL), dried over anhydrous Na₂SO₄, filtered and concentrated under reduced pressure to give a residue. The residue was purified by prep-TLC (SiO₂) to give compound 01-7-1 (90.0 mg, 127.9 μmol, 37% yield). M+H⁺=619.3 (LCMS).

### Step 2: 3-[5-[[4-[(2,6-dichlorophenyl)methyl]piperazin-1-yl]methyl]-3-[4-(tri fluoromethoxy)phenyl]indol-1-yl]-N-methyl-propan-1-amine (01-7)

To a solution of 01-7-1 (60.0 mg, 96.9 μmol, 1.0 eq) in THF (1 mL) was added BH₃*THF (1 M, 290.6 μL, 3.0 eq). The mixture was stirred at 0° C. for 1 h. The reaction mixture was mixed with another batch from 01-7-1 (10 mg). The resulting mixture was quenched by addition of MeOH (3 mL), and then concentrated under reduced pressure to give a residue. The residue was purified by prep-HPLC (FA) to give compound 01-7 (5.6 mg, 8.4 μmol, 9% yield, FA salt). M+H⁺=612.4 (LCMS). ¹H NMR (MeOD, 400 MHz): δ 7.87 (s, 1H), 7.76 (br d, J=8.4 Hz, 2H), 7.58 (s, 1H), 7.50 (br d, J=8.4 Hz, 1H), 7.41-7.32 (m, 4H), 7.27 (br t, J=7.9 Hz, 2H), 4.37 (s, 2H), 3.80 (s, 2H), 3.73 (br s, 2H), 2.98-2.92 (m, 2H), 2.64 (s, 11H), 2.26-2.20 (m, 2H).

### Example 13: Synthesis of N′-[3-[5-[[4-[(2,6-dichlorophenyl)methyl]piperazin-1-yl]methyl]-3-[4-(trifluoromethoxy)phenyl]indol-1-yl]propyl]ethane-1,2-diamine (01-8)

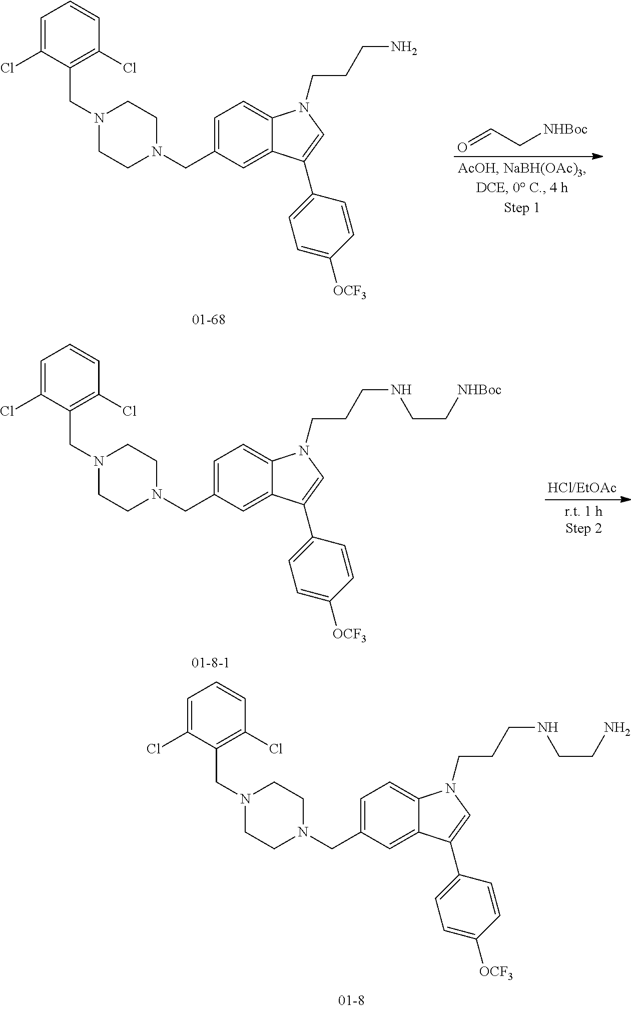 [SMILES: NCCCn1cc(-c2ccc(OC(F)(F)F)cc2)c2cc(CN3CCN(Cc4c(Cl)cccc4Cl)CC3)ccc21 ; CC(C)(C)OC(=O)NCC=O ; CC(C)(C)OC(=O)NCCNCCCn1cc(-c2ccc(OC(F)(F)F)cc2)c2cc(CN3CCN(Cc4c(Cl)cccc4Cl)CC3)ccc21 ; NCCNCCCn1cc(-c2ccc(OC(F)(F)F)cc2)c2cc(CN3CCN(Cc4c(Cl)cccc4Cl)CC3)ccc21]

### Step 1: tert-butyl N-[2-[3-[5-[[4-[(2,6-dichlorophenyl)methyl]piperazin-1-yl]methyl]-3-[4-(trifluoromethoxy)phenyl]indol-1-yl]propylamino]ethyl]carbamate (01-8-1)

A mixture of 01-68 (100.0 mg, 169.1 μmol, 1.0 eq) and tert-butyl N-(2-oxoethyl)carbamate (32.3 mg, 202.9 μmol, 1.2 eq) in DCE (5 mL) was added AcOH (10.1 mg, 169.1 μmol, 9.7 μL, 1.0 eq) and NaBH₃CN (21.3 mg, 338.1 μmol, 2.0 eq) at 0° C., then the mixture was stirred at 0° C. for 4 h. The reaction mixture was added to water (20 mL), basified to pH=8 with NaHCO₃ powder and extracted with DCM (10 mL*3). The combined organic layers were concentrated under reduced pressure and the residue was purified by prep-TLC (SiO₂) to give compound 01-8-1 (30.0 mg, 36.8 μmol, 22% yield).

### Step 2: N′-[3-[5-[[4-[(2,6-dichlorophenyl)methyl]piperazin-1-yl]methyl]-3-[4-(tri fluoromethoxy)phenyl]indol-1-yl]propyl]ethane-1,2-diamine (01-8)

To a mixture of 01-8-1 (50.0 mg, 68.1 μmol, 1.0 eq) in EtOAc (1 mL) was added HCl/EtOAc (4 M, 500.0 μL, 29.4 eq) at room temperature. After the reaction mixture was stirred at room temperature for 1 h, it was concentrated under reduced pressure. The residue was purified by acidic prep-HPLC (HCl condition) to give compound 01-8 (14.0 mg, 20.4 μmol, 30% yield, HCl salt). M+H⁺=634.3 (LCMS). ¹H NMR (MeOD, 400 MHz): δ 8.14 (s, 1H), 7.82 (d, J=8.8 Hz, 2H), 7.76 (s, 1H), 7.72 (d, J=8.4 Hz, 1H), 7.55-7.48 (m, 2H), 7.47-7.40 (m, 2H), 7.36 (d, J=7.9 Hz, 2H), 4.55 (s, 2H), 4.47 (t, J=7.1 Hz, 2H), 4.40 (br s, 2H), 3.48 (br s, 8H), 3.34 (s, 4H), 3.18-3.11 (m, 2H), 2.39-2.31 (m, 2H).

### Example 14: Synthesis of 2-[3-[5-[[4-[(2,6-dichlorophenyl)methyl]piperazin-1-yl]methyl]-3-[4-(trifluoromethoxy)phenyl]indol-1-yl]propylamino]ethanol (01-9)

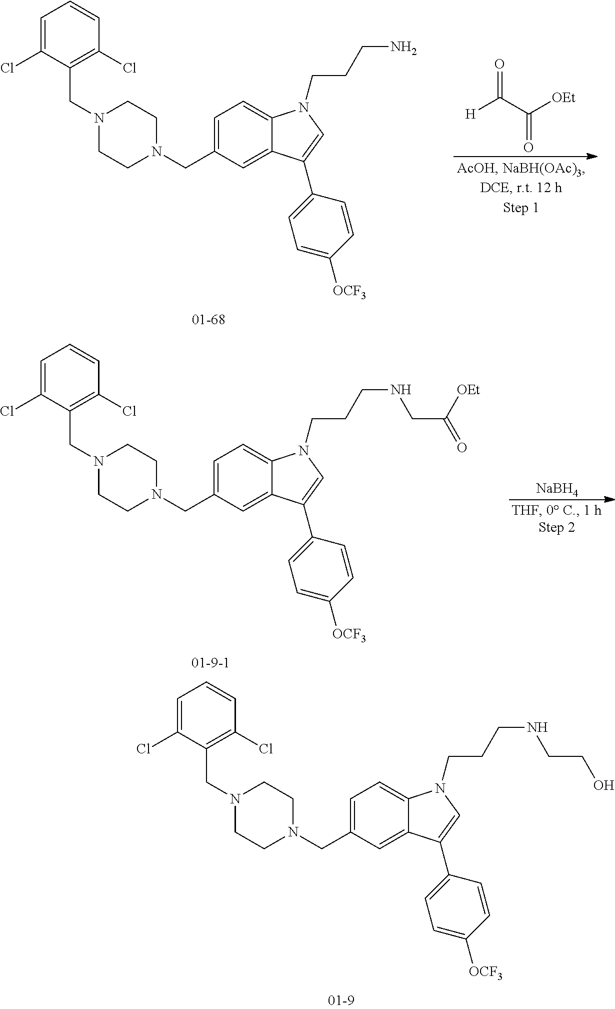 [SMILES: NCCCn1cc(-c2ccc(OC(F)(F)F)cc2)c2cc(CN3CCN(Cc4c(Cl)cccc4Cl)CC3)ccc21 ; CCOC(=O)C=O ; CCOC(=O)CNCCCn1cc(-c2ccc(OC(F)(F)F)cc2)c2cc(CN3CCN(Cc4c(Cl)cccc4Cl)CC3)ccc21 ; "NaBH4" ; OCCNCCCn1cc(-c2ccc(OC(F)(F)F)cc2)c2cc(CN3CCN(Cc4c(Cl)cccc4Cl)CC3)ccc21]

### Step 1: ethyl (3-(5-((4-(2,6-dichlorobenzyl)piperazin-1-yl)methyl)-3-(4-(trifluoro methoxy)phenyl)-1H-indol-1-yl)propyl)glycinate (01-9-1)

To a solution of 01-68 (300.0 mg, 507.2 μmol, 1.0 eq) in DCE (5 mL) was added ethyl 2-oxoacetate (103.6 mg, 507.2 μmol, 1.0 eq), AcOH (30.5 mg, 507.2 μmol, 29.0 μL, 1.0 eq) and NaBH(OAc)₃ (322.5 mg, 1.52 mmol, 3.0 eq). The mixture was stirred at room temperature for 12 h, diluted with H₂O (10 mL) and extracted with DCM (10 mL*3). The combined organic layers were washed with brine (20 mL*2), dried over anhydrous Na₂SO₄, filtered and concentrated under reduced pressure to give a residue. The residue was purified by prep-HPLC (FA condition) to give compound 01-9-1 (200.0 mg, 257.0 μmol, 51% yield, FA salt). M+H⁺=677.3 (LCMS). ¹H NMR (MeOD, 400 MHz): δ 7.84 (s, 1H), 7.75 (br d, J=8.8 Hz, 2H), 7.57 (s, 1H), 7.46 (d, J=8.4 Hz, 1H), 7.39-7.29 (m, 4H), 7.23 (br t, J=8.2 Hz, 2H), 4.31 (br t, J=6.8 Hz, 2H), 4.13 (q, J=7.1 Hz, 2H), 3.78 (s, 2H), 3.67 (s, 2H), 2.66-2.50 (m, 8H), 2.09-2.00 (m, 2H), 1.28 (br s, 2H), 1.21 (t, J=7.1 Hz, 3H), 0.89 (br d, J=7.1 Hz, 2H).

### Step 2: 2-[3-[5-[[4-[(2,6-dichlorophenyl)methyl]piperazin-1-yl]methyl]-3-[4-(trifluoromethoxy)phenyl]indol-1-yl]propylamino]ethanol (01-9)

To a solution of 01-9-1 (90.0 mg, 132.8 μmol, 1.0 eq) in THF (5.00 mL) was added NaBH₄ (15.1 mg, 398.5 μmol, 3.0 eq). The mixture was stirred at 0° C. for 1 h. The reaction mixture was quenched by addition of H₂O (1 mL) at 0° C., diluted with H₂O (10 mL) and extracted with EtOAc (10 mL*3). The combined organic layers were washed with brine (10 mL*2), dried over anhydrous Na₂SO₄, filtered and concentrated under reduced pressure to give a residue. The residue was purified by prep-TLC (SiO₂) to give compound 01-9 (3.7 mg, 4.95 mol, 4% yield). M+H⁺=635.3 (LCMS). ¹H NMR (MeOD, 400 MHz): δ 7.83 (s, 1H), 7.75 (br d, J=8.8 Hz, 2H), 7.55 (s, 1H), 7.46 (br d, J=8.4 Hz, 1H), 7.39-7.28 (m, 4H), 7.26-7.19 (m, 2H), 4.30 (br t, J=6.6 Hz, 2H), 3.77 (s, 2H), 3.66-3.58 (m, 4H), 2.73-2.57 (m, 8H), 2.57-2.39 (m, 4H), 2.12-2.04 (n, 2H).

### Example 15: Synthesis of 3-[5-[[8-[(2-chlorophenyl)methyl]-2,8-diazaspiro[4.5]decan-2-yl]methyl]-3-[4-(trifluoromethoxy)phenyl]indol-1-yl]propan-1-amine (04-48)

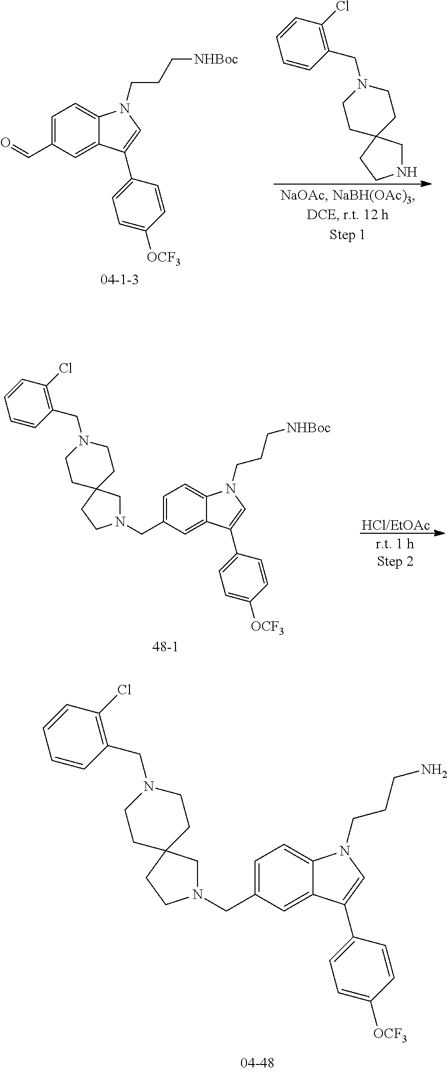 [SMILES: CC(C)(C)OC(=O)NCCCn1cc(-c2ccc(OC(F)(F)F)cc2)c2cc(C=O)ccc21 ; Clc1ccccc1CN1CCC2(CCNC2)CC1 ; CC(C)(C)OC(=O)NCCCn1cc(-c2ccc(OC(F)(F)F)cc2)c2cc(CN3CCC4(CCN(Cc5ccccc5Cl)CC4)C3)ccc21 ; "HCl/EtOAc" ; NCCCn1cc(-c2ccc(OC(F)(F)F)cc2)c2cc(CN3CCC4(CCN(Cc5ccccc5Cl)CC4)C3)ccc21]

### Step 1: tert-butyl N-[3-[5-[[8-[(2-chlorophenyl)methyl]-2,8-diazaspiro[4.5]decan-2-yl]methyl]-3-[4-(trifluoromethoxy)phenyl]indol-1-yl]propyl]carbamate (48-1)

To a solution of 8-[(2-chlorophenyl)methyl]-2,8-diazaspiro[4.5]decane (150.0 mg, 566.5 μmol, 1.0 eq) and tert-butyl N-[3-[5-formyl-3-[4-(trifluoromethoxy)phenyl]indol-1-yl]propyl]carbamate (314.4 mg, 679.8 μmol, 1.2 eq) in DCE (3 mL) was added AcOH (34.0 mg, 566.5 μmol, 32.4 μL, 1.0 eq). The mixture was stirred at room temperature for 2 h under N₂. Then NaBH(OAc)₃ (240.1 mg, 1.1 mmol, 2.0 eq) was added to the mixture portionwise. The resulting mixture was stirred at room temperature for another 10 h, and poured into H₂O (60 mL). The aqueous phase was adjusted to pH 9 with solid NaHCO₃, and extracted with dichloromethane (20 mL*3). The organic layers were washed with brine (40 mL), dried over anhydrous Na₂SO₄, filtered and the filtrate was concentrated in vacuum to give a residue. The residue was purified by prep-TLC (SiO₂) to give compound 48-1 (180.0 mg, 253.1 μmol, 45% yield). ¹H NMR (CDCl₃, 400 MHz): δ 7.75 (s, 1H), 7.59 (d, J=8.7 Hz, 2H), 7.40 (br d, J=7.2 Hz, 1H), 7.29-7.24 (m, 3H), 7.21 (br dd, J=4.5, 7.8 Hz, 3H), 7.12 (dqd, J=1.4, 7.5, 15.6 Hz, 2H), 4.50 (br s, 1H), 4.15 (br t, J=6.8 Hz, 2H), 3.73 (br s, 2H), 3.50 (s, 2H), 3.11 (br d, J=5.8 Hz, 2H), 2.62 (br s, 2H), 2.49-2.24 (m, 6H), 2.05-1.97 (m, 2H), 1.69-1.48 (m, 6H), 1.38 (s, 9H).

### Step 2: 3-[5-[[8-[(2-chlorophenyl)methyl]-2,8-diazaspiro[4.5]decan-2-yl]methyl]-3-[4-(trifluoromethoxy)phenyl]indol-1-yl]propan-1-amine (04-48)

To a solution of tert-butyl N-[3-[5-[[8-[(2-chlorophenyl)methyl]-2,8-diazaspiro[4.5]decan-2-yl]methyl]-3-[4-(trifluoromethoxy)phenyl]indol-1-yl]propyl]carbamate (180.0 mg, 253.1 μmol, 1.0 eq) in EtOAc (1 mL) was added HCl/EtOAc (4 M, 1.0 mL, 15.8 eq). The mixture was stirred at room temperature for 1 h. The reaction mixture was filtered. The filter residue was washed with DCM (5 mL*3). The solid was collected and dried in vacuum to give compound 04-48 (145.0 mg, 223.2 μmol, 88% yield, HCl salt). M+H⁺=611.2 (LCMS). ¹H NMR (MeOD, 400 MHz): δ 8.18 (br d, J=19.0 Hz, 1H), 7.86-7.81 (m, 2H), 7.77-7.71 (m, 2H), 7.69-7.65 (m, 1H), 7.59-7.55 (m, 1H), 7.54-7.43 (m, 3H), 7.36 (br dd, J=4.2, 7.7 Hz, 2H), 4.60 (br d, J=5.3 Hz, 1H), 4.56-4.51 (m, 3H), 4.46-4.40 (m, 2H), 3.77-3.57 (m, 2H), 3.56-3.40 (m, 4H), 3.27-3.17 (m, 2H), 3.00-2.94 (m, 2H), 2.34-2.21 (m, 3H), 2.18-2.10 (m, 2H), 2.04-1.87 (m, 3H).

### Example 16: Synthesis of 1-[(2,5-dichlorophenyl)methyl]piperazine (05-36-2)

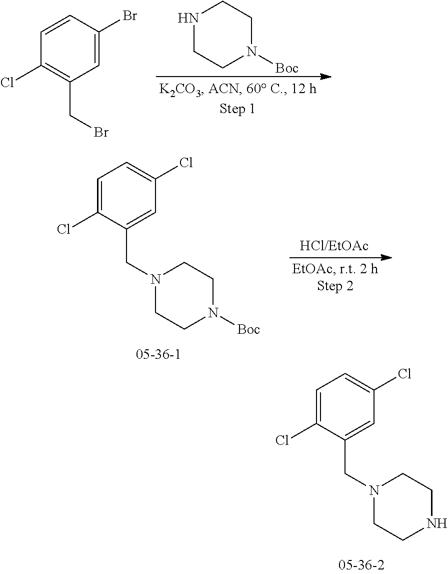

### Step 1: tert-butyl 4-[(2,5-dichlorophenyl)methyl]piperazine-1-carboxylate (05-36-1)

To a mixture of 1,4-dichloro-2-(chloromethyl)benzene (1.00 g, 5.12 mmol, 1.0 eq) in ACN (10 mL) was added tert-butyl piperazine-1-carboxylate (952.8 mg, 5.12 mmol, 1.0 eq) and K₂CO₃ (1.42 g, 10.2 mmol, 2.0 eq). The mixture was stirred at 60° C. for 12 h. The mixture was filtered and concentrated under reduced pressure to give a residue. The residue was purified by column chromatography (SiO₂) to give compound 05-36-1 (1.50 g, 82.3% yield). M+H⁺=345.1 (LCMS).

### Step 2: 1-[(2,5-dichlorophenyl)methyl]piperazine (05-36-2)

To a mixture of 05-36-1 (1.50 g, 1.00 eq) in EtOAc (2 mL) was added HCl/EtOAc (4 M, 10 mL). The mixture was stirred at room temperature for 2 h. The mixture was concentrated under reduced pressure to give a residue, MeOH (10 mL) and H₂O (5 mL) was added, followed by addition of AMBERLYST® A21 to pH>8, then the mixture was filtered and concentrated under reduced pressure to give compound 05-36-2 (1.00 g, 4.08 mmol, 94% yield). M+H⁺=245.1 (LCMS).

### Example 17: Synthesis of 4-(2,2,2-trifluoroethyl)piperidine (04-55-3)

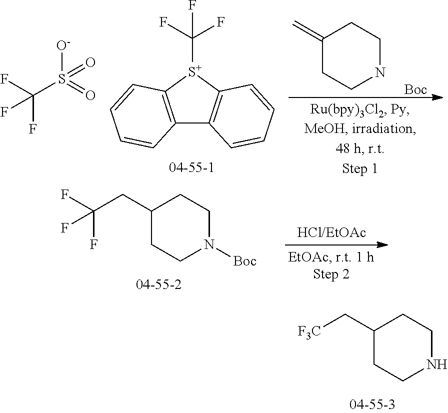

### Step 1: tert-butyl 4-(2,2,2-trifluoroethyl)piperidine-1-carboxylate (04-55-2)

To a solution of tert-butyl 4-methylenepiperidine-1-carboxylate (200.0 mg, 1.01 mmol, 1.2 eq) in MeOH (2 mL) was added pyridine (80.2 mg, 1.01 mmol, 81.8 μL, 1.2 eq), dichlororuthenium 2-(2-pyridyl)pyridine (27.1 mg, 42.2 μmol, 0.05 eq) and trifluoromethanesulfonate 5-(trifluoromethyl)dibenzothiophen-5-ium (407.9 mg, 1.01 mmol, 1.2 eq). The vial was exposed to a fluorescent light bulb (14 W) at room temperature while stirring for 48 h. The reaction mixture was concentrated in vacuum to give a residue. The residue was dissolved in DCM (8 mL), and diluted with citric acid aqueous solution (30 mL). The mixture was extracted with DCM (10 mL*3). The organic phase was washed with brine (20 mL), dried over anhydrous Na₂SO₄, filtered and the filtrate was concentrated in vacuum to give the crude product. The crude product was purified by column chromatography (SiO₂) to give compound 04-55-2 (180.0 mg, crude). ¹H NMR (CDCl₃, 400 MHz): δ 4.27-3.83 (m, 2H), 3.55-3.24 (m, 1H), 2.90-2.61 (m, 2H), 2.30-1.96 (m, 2H), 1.89-1.71 (m, 2H), 1.49-1.44 (m, 9H), 1.36-1.14 (n, 2H).

### Step 2: 4-(2, 2, 2-trifluoroethyl)piperidine (04-55-3)

To a solution of tert-butyl 4-(2,2,2-trifluoroethyl)piperidine-1-carboxylate (180.0 mg, 673.4 μmol, 1.0 eq) in EtOAc (1 mL) was added HCl/EtOAc (4 M, 1.0 mL, 5.9 eq). The mixture was stirred at room temperature for 1 h. The reaction mixture was poured into H₂O (30 mL). Then the aqueous phase was adjusted to pH 9 with solid NaOH, and extracted with dichloromethane (20 mL*3). The organic phase was washed with brine (30 mL), dried over anhydrous Na₂SO₄, filtered and the filtrate was concentrated in vacuum to give compound 4-(2,2,2-trifluoroethyl)piperidine (90.00 mg, crude). M+H⁺=168.2 (LCMS).

### Example 18: Synthesis of 4-ethylpiperidine (04-64-1)

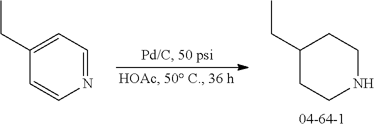

To a solution of 4-ethylpyridine (5.00 g, 46.7 mmol, 5.32 mL, 1.0 eq) in AcOH (100.0 mL) was added Pd/C (10%, 3 g) under N₂ atmosphere. The suspension was degassed and purged with H₂ for 3 times. The mixture was stirred under H₂ (50 Psi) at 50° C. for 36 hr. The mixture was filtered, the filtrate was concentrated under reduced pressure to afford a residue. The residue was redissolved in DCM (30 ml) and adjusted to pH=11 by addition of aq NaOH (1 N). The organic layer was separated and the aqueous phase was extracted with DCM (20 mL), the combined organic layers were concentrated under reduced pressure to give the compound 04-64-1 (5.10 g, 42.8 mmol, 92% yield). ¹H NMR (MeOD, 400 MHz): δ 2.96-2.99 (m, 2H) 2.46-2.52 (m, 2H) 1.71 (br s, 1H) 1.58-1.61 (m, 2H) 1.17-1.18 (m, 3H) 0.97-1.03 (m, 2H) 0.79-0.82 (m, 3H).

### Example 19: Synthesis of N-(4-chlorophenyl)piperidin-4-amine (04-58-2)

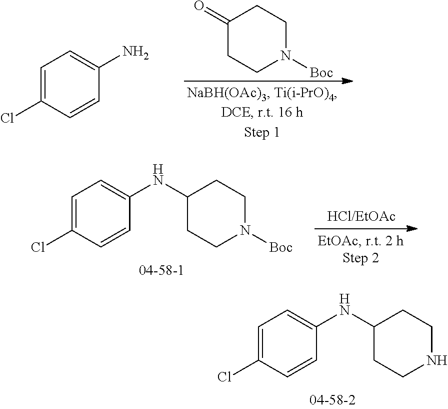 [SMILES: Nc1ccc(Cl)cc1 ; CC(C)(C)OC(=O)N1CCC(=O)CC1 ; CC(C)(C)OC(=O)N1CCC(Nc2ccc(Cl)cc2)CC1 ; Clc1ccc(NC2CCNCC2)cc1]

### Step 1: tert-butyl 4-(2-chloroanilino)piperidine-1-carboxylate (04-58-1)

To a solution of 4-chloroaniline (1.00 g, 7.84 mmol, 1.0 eq) in DCE (15 mL) was added tert-butyl 4-oxopiperidine-1-carboxylate (1.56 g, 7.84 mmol, 1.0 eq) and Ti(i-PrO)₄ (2.23 g, 7.84 mmol, 2.32 mL, 1.0 eq). The mixture was stirred at room temperature for 14 hr, then NaBH(OAc)₃ (1.66 g, 7.84 mmol, 1.0 eq) was added in one portion. The mixture was stirred at room temperature for another 2 h. The mixture was quenched by addition of H₂O (1.0 mL), diluted with DCM (50 mL) and stirred for 0.5 h. The mixture was filtered and the filtrate was washed with H₂O (20 mL), brine (20 mL), dried over anhydrous Na₂SO₄ and filtered. The filtrate was concentrated under reduced pressure to afford a residue. The residue was purified by column chromatography (SiO₂) to give compound 04-58-1 (2.10 g, 65% yield). ¹H NMR (CDCl₃, 400 MHz): δ 7.01-7.05 (m, 2H), 6.43-6.45 (m, 2H), 3.96-4.01 (m, 2H), 3.43 (m, 2H), 3.28-3.32 (m, 1H), 2.81-2.87 (m, 2H), 1.93-1.97 (m, 2H), 1.39 (s, 9H).

### Step 2: N-(4-chlorophenyl)piperidin-4-amine (4-58-2)

To a solution of tert-butyl 4-(4-chloroanilino)piperidine-1-carboxylate (2.10 g, 1.0 eq) in EtOAc (20.00 mL) was added HCl/EtOAc (4 M, 20 mL, 11.8 eq) dropwise slowly at 0° C. After addition the mixture was stirred at room temperature for 2 h. The mixture was filtered and the filter cake was washed with EtOAc (10 mL*2). The solid was redissolved in MeOH (20 mL) and stirred with K₂CO₃ (2.0 g) at room temperature for 0.5 h, then filtered. The filtrate was concentrated under reduced pressure to afford compound N-(4-chlorophenyl)piperidin-4-amine (1.36 g, 6.13 mmol, 91% yield). ¹H NMR (MeOD, 400 MHz): δ 7.03-7.05 (m, 2H), 6.60-6.64 (m, 2H), 3.52 (m, 1H), 3.34-3.37 (m, 2H), 3.04-3.07 (m, 2H), 2.13-2.17 (m, 2H), 1.57-1.62 (m, 2H).

### Example 20: Synthesis of 4-(2-chlorophenoxy)piperidine (04-60-2)

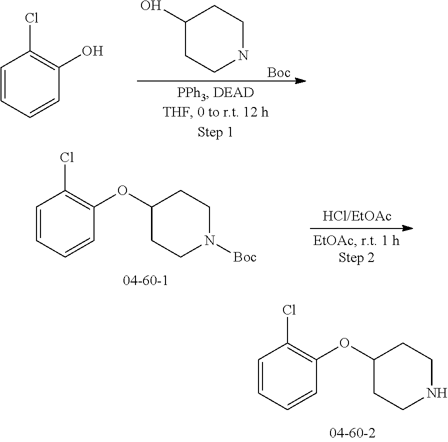 [SMILES: Oc1ccccc1Cl ; OC1CCNCC1 ; CC(C)(C)OC(=O)N1CCC(Oc2ccccc2Cl)CC1 ; Clc1ccccc1OC1CCNCC1 ; "Boc"]

### Step 1: tert-butyl 4-(2-chlorophenoxy) piperidine-1-carboxylate (04-60-1)

To a mixture of 2-chlorophenol (1.00 g, 7.78 mmol, 793.65 μL, 1.00 eq) in THF (10 mL) was added tert-butyl 4-hydroxypiperidine-1-carboxylate (1.72 g, 8.56 mmol, 1.10 eq) and PPh₃ (2.45 g, 9.34 mmol, 1.20 eq). Then DEAD (1.63 g, 9.34 mmol, 1.69 mL, 1.20 eq) was added to the mixture at 0° C. Then the mixture was stirred at 0° C. for 0.5 h. Then the mixture was stirred at room temperature for 11.5 h. The mixture was concentrated under reduced pressure to give a residue. Then the mixture was washed by H₂O (20 mL), and extracted with MTBE (20 mL*2). The combined organic layers were washed with brine (20 mL*2), dried over anhydrous Na₂SO₄, filtered and concentrated under reduced pressure to give compound 04-60-1 (1.00 g, 40% yield). M+H⁺=256.1 (LCMS).

### Step 2: 4-(2-chlorophenoxy)piperidine (04-60-2)

To a mixture of tert-butyl 4-(2-chlorophenoxy)piperidine-1-carboxylate (1.00 g, 1.00 eq) in EtOAc (2.00 mL) was added HCl/EtOAc (4 M, 5 mL). The mixture was stirred at room temperature for 1 h. The mixture was concentrated under reduced pressure to give a residue. Then the residue was poured to MeOH (20 mL), and then K₂CO₃ was added to the mixture to make the pH to 9, the mixture was filtered and concentrated under reduced pressure to give a residue. The crude product 4-(2-chlorophenoxy)piperidine (500.0 mg, crude) was used into the next step without further purification. M+H⁺=212.1 (LCMS).

### Example 21: Synthesis of N′-[(2-chlorophenyl)methyl]-N′-methyl-ethane-1,2-diamine (08-6-2)

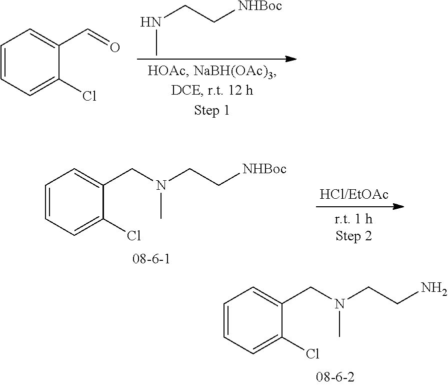

### Step 1: tert-butyl N-[2-[(2-chlorophenyl)methyl-methyl-amino]ethyl]carbamate (08-6-1)

To a solution of 2-chlorobenzaldehyde (1.00 g, 7.11 mmol, 800.0 μL, 1.0 eq) in DCE (10 mL) was added tert-butyl N-[2-(methylamino)ethyl]carbamate (1.36 g, 7.82 mmol, 1.1 eq), AcOH (427.0 mg, 7.11 mmol, 406.7 μL, 1.0 eq) and NaBH(OAc)₃ (4.52 g, 21.3 mmol, 3.0 eq). The mixture was stirred at room temperature for 12 h, poured into H₂O (50 mL) and extracted with DCM (50 mL*3). The combined organic layers were washed with Na₂CO₃ aq. (50 mL*2), brine (50 mL*2), dried over anhydrous Na₂SO₄, filtered and concentrated under reduced pressure to give compound 08-6-1 (1.80 g, crude), which was used into the next step without further purification.

### Step 2: N′-[(2-chlorophenyl)methyl]-N′-methyl-ethane-1,2-diamine (08-6-2)

To a solution of 08-6-1 (1.80 g, 6.02 mmol, 1.0 eq) in EtOAc (10 mL) was added HCl in EtOAc (4 M, 10.0 mL, 6.6 eq). The mixture was stirred at room temperature for 1 h and concentrated. The residue was dissolved in H₂O (20 mL), and adjusted pH to 8 with Na₂CO₃ and extracted with EtOAc (50 mL*3). The combined organic layers were washed with brine (100 mL*2), dried over anhydrous Na₂SO₄, filtered and concentrated under reduced pressure to give compound 08-6-2 (900.0 mg, 4.53 mmol, 75% yield), which was used without further purification.

### Example 22: Synthesis of N′-[(2-chlorophenyl)methyl]-N,N′-dimethyl-ethane-1,2-diamine (08-7-1)

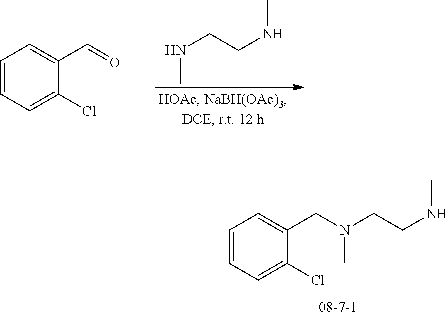

To a solution of 2-chlorobenzaldehyde (1.00 g, 7.11 mmol, 800.0 μL, 1.0 eq) in MeOH (5 mL) was added N,N′-dimethylethane-1,2-diamine (626.8 mg, 7.11 mmol, 764.3 μL, 1.00 eq), tetraisopropoxytitanium (2.02 g, 7.11 mmol, 2.10 mL, 1.0 eq) and NaBH₃CN (670.2 mg, 10.7 mmol, 1.5 eq). The mixture was stirred at room temperature for 12 h, diluted with H₂O (20 mL) and EtOAc (20 mL) and filtered through Celite. The filtrate was extracted with EtOAc (50 mL*2) and the combined organic layers were washed with brine (100 mL*2), dried over anhydrous Na₂SO₄, filtered and concentrated to give a residue which was purified by column chromatography (SiO₂) to give compound 08-7-1 (300.0 mg, 1.31 mmol, 19% yield). M+H⁺=171.1 (LCMS). ¹H NMR (MeOD, 400 MHz): δ 7.76 (dd, J=1.8, 7.7 Hz, 1H), 7.37-7.28 (m, 2H), 7.24 (dd, J=1.7, 7.5 Hz, 1H), 4.11 (s, 1H), 3.45-3.33 (m, 2H), 2.72-2.59 (m, 2H), 2.23 (s, 6H)

The following compounds were synthesized from intermediate 04-1-3 using procedures described in Steps 1 and 2 above for the preparation of compound 04-48.

| Comp | | | Mass | ¹H NMR (MeOD, |
|---|---|---|---|---|
| ID | Structure | Chemical Name | (M + H⁺) | 400 MHz): |
| 04-45 | 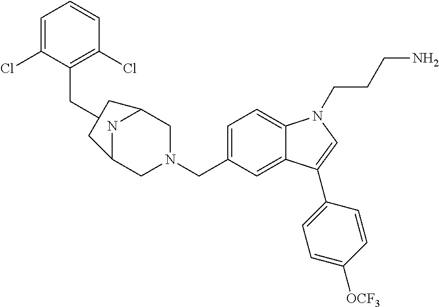 [SMILES: NCCCn1cc(-c2ccc(OC(F)(F)F)cc2)c2cc(CN3CC4CC(Cc5c(Cl)cccc5Cl)CC(C3)N4)ccc21] | 3-(5-((8-(2,6- dichlorobenzyl)- 3,8- diazabicyclo[3.2.1] octan-3- yl)methyl)-3-(4- (trifluoromethoxy) phenyl)-1H-indol- 1-yl)propan-1- amine hydrochloride | Calc'd for C₃₂H₃₄Cl₂F₃N₄O: 617.2: Found: 617.3 | δ 8.45 (s, 1H), 7.73-7.71 (m, 2H), 7.63-7.61 (m, 1H), 7.54-7.50 (m, 1H), 7.45-7.43 (m, 5H), 7.30-7.24 (m, 2H), 4.45 (p, 4H), 4.33-4.30 (t, 4H), 3.77-3.74 (m, 2H), 3.48-3.34 (m, 2H), 2.88-2.84 (m, 2H), 2.51-2.47 (m, 4H), 2.18-2.11 (m, 2H) |
| 04-44 | 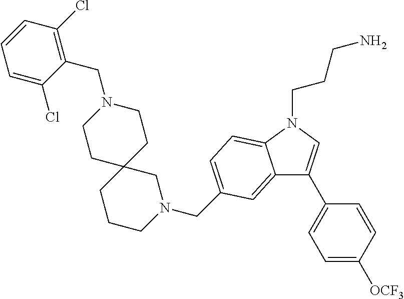 [SMILES: NCCCn1cc(-c2ccc(OC(F)(F)F)cc2)c2cc(CN3CCCC4(CCN(Cc5c(Cl)cccc5Cl)CC4)C3)ccc21] | 3-(5-((9-(2,6- dichlorobenzyl)- 2,9- diazaspiro[5.5] undecan-2-yl) methyl)- 3-(4- (trifluoromethoxy) phenyl)-1H-indol- 1-yl)propan-1- amine hydrochloride | Calc'd for C₃₅H₅₀Cl₂F₃N₄O: 659.2; Found: 659.3 | δ 8.17 (s, 1H), 7.72-7.69 (m, 2H), 7.66-7.61 (m, 2H), 7.48-7.45 (m, 2H), 7.40-7.36 (m, 2H), 7.26-7.24 (m, 2H), 4.59-4.55 (m, 3H), 4.43-4.32 (m, 4H), 4.01-3.97 (br, 1H), 3.48-3.328 (m, 5H), 3.20-2.97 (m, 5H), 2.59-2.38 (m, 1H), 2.31-2.27 (m, 1H), 2.16 (s, 2H), 1.89-1.84 (m, 4H), 1.60-1.51 (m, 1H), 1.42-1.27 (m, 1H) |
| 05-32 | 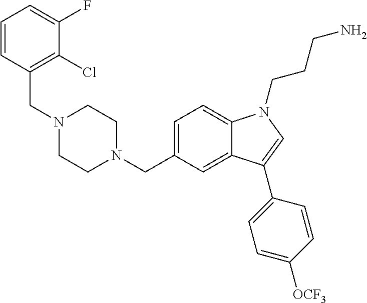 [SMILES: NCCCn1cc(-c2ccc(OC(F)(F)F)cc2)c2cc(CN3CCN(Cc4cccc(F)c4Cl)CC3)ccc21] | 3-(5-((4-(2-chloro- 3-fluorobenzyl) piperazin-1- yl)methyl)-3-(4- (trifluoromethoxy) phenyl)-1H-indol- 1-yl)propan-1- amine hydrochloride | Calc'd for C₃₀H₃₂ClF₄N₄O: 575.2; Found: 575.3 | δ 8.20 (s, 1H), 7.84 (d, J = 8.8 Hz, 2H), 7.72 (s, 1H), 7.70- 7.62 (m, 2H), 7.52- 7.32 (m, 5H), 4.61 (br d, J = 7.5 Hz, 4H), 4.42 (br t, J = 6.8 Hz, 2H), 3.69 (br s, 8H), 3.01- 2.92 (m, 2H), 2.30- 2.20 (m, 2H) |
| 04-46 | 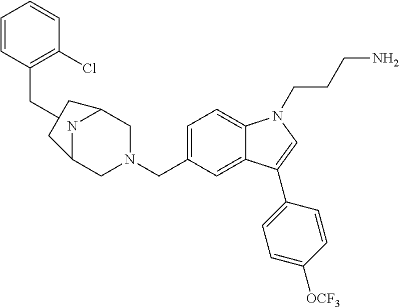 [SMILES: NCCCn1cc(-c2ccc(OC(F)(F)F)cc2)c2cc(CN3CC4CC(Cc5ccccc5Cl)CC(C3)N4)ccc21] | 3-(5-((8-(2- chlorobenzyl)-3,8- diazabicyclo[3.2.1] octan-3- yl)methyl)-3-(4- (trifluoromethoxy) phenyl)-1H-indol- 1-yl)propan-1- amine hydrochloride | Calc'd for C₃₂H₃₅ClF₃N₄O: 583.2; Found: 583.3 | δ 8.18 (s, 1H), 7.90-7.82 (p, 1H), 7.80-7.72 (m, 2H), 7.69-7.63 (m, 1H), 7.60-7.54 (m, 1H), 7.49-7.48 (m, 1H), 7.46-7.38 (m, 3H), 7.36-7.34 (m, 2H), 4.48-4.43 (m, 6H), 4.41 (s, 2H), 4.01- 3.95 (br s, J = 24 Hz, 2H), 3.54-3.52 (m, 2H), 2.97-2.93 (m, 2H), 2.61-2.50 (m, 4H), 2.27-2.20 (m, 2H) |
| 04-49 | 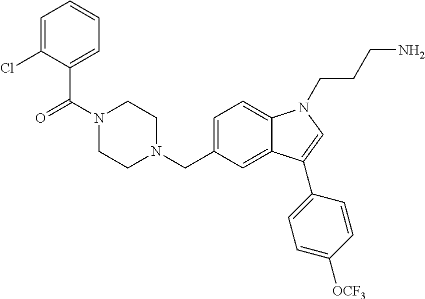 [SMILES: NCCCn1cc(-c2ccc(OC(F)(F)F)cc2)c2cc(CN3CCN(C(=O)c4ccccc4Cl)CC3)ccc21] | [4-[[1-(3- aminopropyl)-3- [4- (trifluoromethoxy) phenyl]indol-5- yl]methyl] piperazin-1-yl]-(2- chlorophenyl) methanone hydrochloride | Calc'd for C₃₀H₃₁ClF₃N₄O₂: 571.2: Found: 571.3 | δ 8.11 (br s, 1H), 7.79 (d, J = 8.8 Hz, 2H), 7.71-7.65 (m, 2H), 7.53-7.41 (m, 5H), 7.36 (d, J = 7.9 Hz, 2H), 4.93- 4.89 (m, 2H), 4.52 (br s, 2H), 4.42 (t, J = 6.9 Hz, 2H), 3.59 (br s, 1H), 3 31 (s, 5H), 3.00-2.93 (m, 2H), 2.28-2.19 (m, 2H) |
| 04-50 | 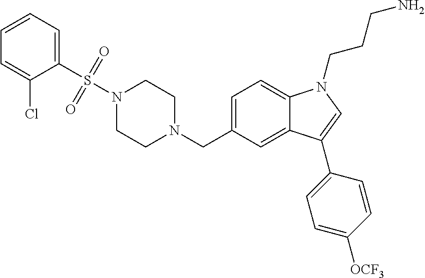 [SMILES: NCCCn1cc(-c2ccc(OC(F)(F)F)cc2)c2cc(CN3CCN(S(=O)(=O)c4ccccc4Cl)CC3)ccc21] | 3-[5-[[4-(2- chlorophenyl) sulfonylpiperazin- 1-yl]methyl]-3-[4- (trifluoromethoxy) phenyl]indol-1- yl]propan-1-amine hydrochloride | Calc'd for C₂₉H₃₁ClF₃N₄O₃S: 607.2; Found: 607.3 | δ 8.12-8.07 (m, 2H), 7.80 (d, J = 8.5 Hz, 2H), 7.72 (s, 1H), 7.69 (d, J = 8.5 Hz, 1H), 7.66 (d, J = 3.6 Hz, 2H), 7.57-7.51 (m, 1H), 7.44 (d, J = 8.0 Hz, 1H), 7.38 (d, J = 8.2 Hz, 2H), 4.54 (s, 2H), 4.44 (t, J = 7.0 Hz, 2H), 4.04 (br s, 2H), 3.62- 3.47 (m, 2H), 3.24 (br d, J = 4.0 Hz, 4H), 3.03-2.94 (m, 2H), 2.31-2.21 (m, |
| | | | | 2H) |
| 05-31 | 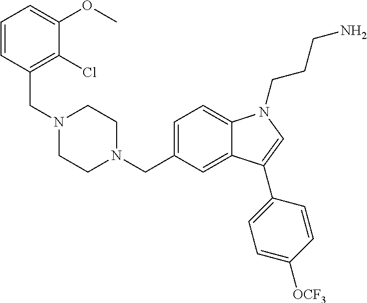 [SMILES: COc1cccc(CN2CCN(Cc3ccc4c(c3)c(-c3ccc(OC(F)(F)F)cc3)cn4CCCN)CC2)c1Cl] | 3-(5-((4-(2-chloro- 3- methoxybenzyl) piperazin-1- yl)methyl)-3-(4- (trifluoromethoxy) phenyl)-1H-indol- 1-yl)propan-1- amine hydrochloride | Calc'd for C₃₁H₃₅ClF₃N₄O₂: 587.2: Found: 587.2 | δ 8.16 (s, 1H), 7.81 (d, J = 8.7 Hz, 2H), 7.71-7.66 (m, 2H), 7.48 (br d, J = 7.9 Hz, 1H), 7.36 (br d, J = 8.4 Hz, 3H), 7.28 (br d, J = 7.7 Hz, 1H), 7.20 (br d, J = 8.3 Hz, 1H), 4.58 (s, 2H), 4.49- 4.45 (m, 2H), 4.42 (br t, J = 6.9 Hz, 2H), 3.91 (s, 3H), 3.59 (br s, 8H), 3.01-2.92 (m, 2H), 2.31-2.19 (m, 2H) |
| 05-39 | 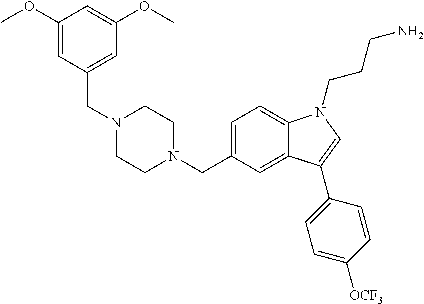 [SMILES: COc1cc(CN2CCN(Cc3ccc4c(c3)c(-c3ccc(OC(F)(F)F)cc3)cn4CCCN)CC2)cc(OC)c1] | 3-(5-((4-(3,5- dimethoxybenzyl) piperazin-1- yl)methyl)-3-(4- (trifluoromethoxy) phenyl)-1H-indol- 1-yl)propan-1- amine hydrochloride | Calc'd for C₃₂H₃₈F₃N₄O₃: 583.3: Found: 583.2 | δ 8.18 (s, 1H), 7.85-7.79 (m, 2H), 7.70 (s, 1H), 7.67 (d, J = 8.5 Hz, 1H), 7.51-7.46 (m, 1H), 7.36 (br d, J = 8.0 Hz, 2H), 6.79 (d, J = 2.1 Hz, 2H), 6.57 (t, J = 2.2 Hz, 1H), 4.61 (s, 2H), 4.45- 4.35 (m, 4H), 3.82- 3.79 (m, 6H), 3.75- 3.58 (m, 8H), 2.99- 2.93 (m, 2H), 2.30- 2.20 (m, 2H) |
| 05-29 | 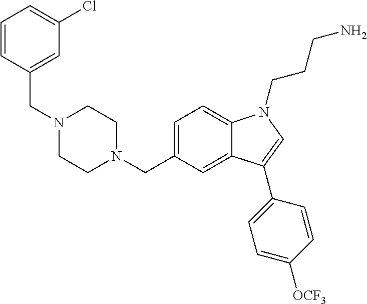 [SMILES: NCCCn1cc(-c2ccc(OC(F)(F)F)cc2)c2cc(CN3CCN(Cc4cccc(Cl)c4)CC3)ccc21] | 3-(5-((4-(3- chlorobenzyl) piperazin-1-yl) methyl)-3-(4- (trifluoromethoxy) phenyl)-1H-indol- 1-yl)propan-1- amine hydrochloride | Calc'd for C₃₀H₃₃ClF₃N₄O: 557.2; Found: 557.3 | δ 8.18 (s, 1H), 7.83 (d, J = 8.66 Hz, 2H), 7.71 (s, 1H), 7.68-7.64 (m, 2H), 7.53-7.45 (m, 4H), 7.36 (br d, J = 8.16 Hz, 2H), 4.60 (s, 2 H), 4.42 (br t, J = 6.90 Hz, 2H), 4.37 (br s, 2H), 3.60 (br d, J = 4.52 Hz, 8H), 3.00-2.93 (m, 2H), 2.29-2.21 (m, 2H) |
| 05-37 | 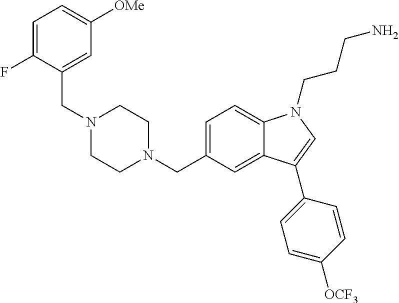 [SMILES: COc1ccc(F)c(CN2CCN(Cc3ccc4c(c3)c(-c3ccc(OC(F)(F)F)cc3)cn4CCCN)CC2)c1] | 3-(5-((4-(2-fluoro- 5- methoxybenzyl) piperazin-1- yl)methyl)-3-(4- (trifluoromethoxy) phenyl)-1H-indol- 1-yl)propan-1- amine hydrochloride | Calc'd for C₃₁H₃₅F₄N₄O₂: 571.3: Found: 571.4 | δ 8.19 (s, 1H), 7.83 (d, J = 8.66 Hz, 2H), 7.71 (s, 1H), 7.67 (d, J = 8.41 Hz, 1H), 7.49 (br d, J = 8.78 Hz, 1H), 7.35 (br d, J = 8.16 Hz, 2H), 7.27 (dd, J = 5.65, 3.14 Hz, 1H), 7.20-7.13 (m, 1H), 7.12-7.06 (m, 1H), 7.08-7.02 (m, 1H), 4.62 (s, 2H), 4.49 (br s, 2H), 4.44-4.38 (m, 2H), 3.82 (s, 3H), 3.71 (br s, 8H), 2.99- 2.92 (m, 2H), 2.25 (quin, J = 7.34 Hz, |
| | | | | 2H) |
| 04-51 | 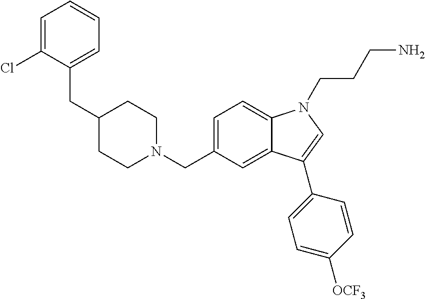 | 3-[5-[[4-[(2- chlorophenyl) methyl]-1- piperidyl]methyl- 3-[4- (trifluoromethoxy) phenyl]indol-1- yl]propan-1-amine hydrochloride | Calc'd for C₅₁H₃₄ClF₃N₃O: 556.2; Found: 556.3 | δ 8.07 (s, 1H), 7.78 (br d, J = 8.4 Hz, 2H), 7.72-7.69 (m, 1H), 7.66 (d, J = 8.4 Hz, 1H), 7.40 (br d, J = 8.4 Hz, 1H), 7.36 (br d, J = 7.5 Hz, 3H), 7.27- 7.16 (m, 3H), 4.48- 4.34 (m, 4H), 3.47 (br d, J = 12.3 Hz, 2H), 3.03-2.90 (m, 4H), 2.74 (br d, J = 7.1 Hz, 2H), 2.29- 2.19 (m, 2H), 1.99 (br d, J = 3.5 Hz, 1H), 1.87 (br d, J = 14.6 Hz, 2H), 1.64- 1.50 (m, 2H) |
| 04-52 | 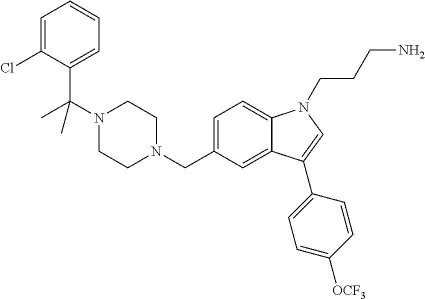 [SMILES: CC(C)(c1ccccc1Cl)N1CCN(Cc2ccc3c(c2)c(-c2ccc(OC(F)(F)F)cc2)cn3CCCN)CC1] | 3-[5-[[4-[1-(2- chlorophenyl)-1- methyl- ethyl]piperazin-1- yl]methyl]-3-[4- (trifluoromethoxy) phenyl]indol-1- yl]propan-1-amine hydrochloride | Calc'd for C₃₂H₃₇ClF₃N₄O: 585.3; Found: 585.3 | δ 8.48 (s, 1H), 7.99 (s, 1H), 7.78-7.74 (m, 2H), 7.63 (s, 1H), 7.58 (d, J = 8.6 Hz, 1H), 7.51 (dd, J = 1.7, 7.8 Hz, 1H), 7.38-7.32 (m, 4H), 7.27-7.16 (m, 2H), 4.39 (t, J = 6.8 Hz, 2H), 4.15 (br s, 2H), 3.15-2.90 (m, 6H), 2.69 (br s, 4H), 2.22 (quin, J = 7.5 Hz, 2H), 1.52 (s, 6H) |
| 05-33 | 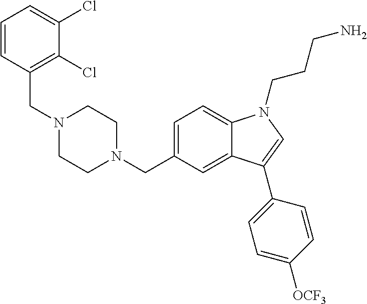 [SMILES: NCCCn1cc(-c2ccc(OC(F)(F)F)cc2)c2cc(CN3CCN(Cc4cccc(Cl)c4Cl)CC3)ccc21] | 3-(5-((4-(2,3- dichlorobenzyl) piperazin-1- yl)methyl)-3-(4- (trifluoromethoxy) phenyl)-1H-indol- 1-yl)propan-1- amine hydrochloride | Calc'd for C₃₀H₃₂Cl₂F₃N₄O: 591.2; Found: 591.3 | δ 8.19 (s, 1H), 7.83 (d, J = 8.66 Hz, 2H), 7.73-7.66 (m, 2H), 7.61-7.55 (m, 2H), 7.49 (br d, J = 8.53 Hz, 1H), 7.40 (br d, J = 7.15 Hz, 1H), 7.35 (br d, J = 8.16 Hz, 2H), 4.61 (s, 2H), 4.48- 4.40 (m, 4H), 3.64 (br s, 8H), 2.99- 2.93 (m, 2H), 2.29- 2.21 (m, 2H) |
| 05-30 | 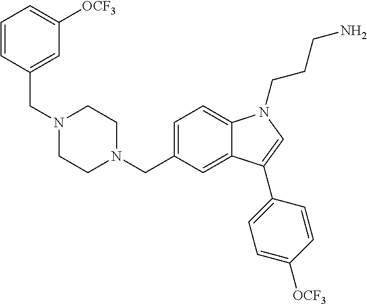 [SMILES: NCCCn1cc(-c2ccc(OC(F)(F)F)cc2)c2cc(CN3CCN(Cc4cccc(OC(F)(F)F)c4)CC3)ccc21] | 3-(5-((4-(3- (trifluoromethoxy) benzyl)piperazin- 1-yl)methyl)-3-(4- (trifluoromethoxy) phenyl)-1H-indol- 1-yl)propan-1- amine hydrochloride | Calc'd for C₃₁H₃₃F₆N₄O₂: 607.2: Found: 607.3 | δ 8.19 (s, 1H), 7.83 (d, J = 8.66 Hz, 2 H), 7.73-7.66 (m, 2 H), 7.64-7.57 (m, 3 H), 7.49 (br d, J = 8.53 Hz, 1H), 7.43- 7.34 (m, 3 H), 4.61 (s, 2 H), 4.48-4.39 (m, 4H), 3.64 (br s, 8H), 2.99-2.93 (m, 2H), 2.29-2.21 (m, 2H) |
| 05-36 | 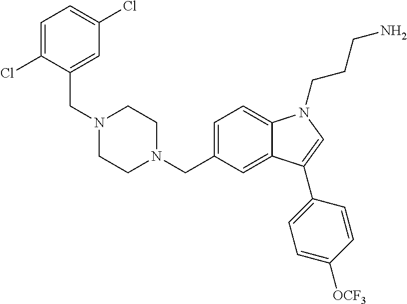 [SMILES: NCCCn1cc(-c2ccc(OC(F)(F)F)cc2)c2cc(CN3CCN(Cc4cc(Cl)ccc4Cl)CC3)ccc21] | 3-(5-((4-(2,5- dichlorobenzyl) piperazin-1- yl)methyl)-3-(4- (trifluoromethoxy) phenyl)-1H-indol- 1-yl)propan-1- amine hydrochloride | Calc'd for C₃₀H₃₂Cl₂F₃N₄O: 591.2; Found: 591.3 | δ 8.21 (br s, 1H), 7.88-7.81 (m, 3H), 7.77-7.72 (m, 1H), 7.69 (br d, J = 8.38 Hz, 1H), 7.57- 7.49 (m, 3H), 7.36 (br d, J = 8.38 Hz, 2H), 4.66-4.57 (m, 2H), 4.48 (br s, 2H), 4.40 (br s, 1H), 3.68 (br s, 6H), 3.01-2.92 (m, 2H), 2.33-2.24 (m, 2H), 1.31-1.30 (m, 2H), 1.29 (s, 4H) |
| 05-34 | 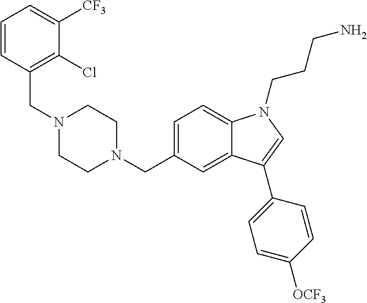 [SMILES: NCCCn1cc(-c2ccc(OC(F)(F)F)cc2)c2cc(CN3CCN(Cc4cccc(C(F)(F)F)c4Cl)CC3)ccc21] | 3-(5-((4-(2-chloro- 3- (trifluoromethyl) benzyl)piperazin-1- yl)methyl)-3-(4- (trifluoromethoxy) phenyl)-1H-indol- 1-yl)propan-1- amine hydrochloride | Calc'd for C₃₁H₃₂ClF₆N₄O: 625.2; Found: 625.5 | δ 8.19 (s, 1H), 8.06 (br d, J = 7.5 Hz, 1H), 7.93 (br d, J = 7.9 Hz, 1H), 7.88- 7.80 (m, 2H), 7.75- 7.61 (m, 3H), 7.49 (br d, J = 8.8 Hz, 1H), 7.36 (br d, J = 7.9 Hz, 2H), 4.72- 4.51 (m, 4H), 4.42 (br t, J = 6.8 Hz, 2H), 3.75-3.52 (m, 6H), 3.00-2.90 (m, 2H), 2.29-2.20 (m, 2H), 2.07-1.92 (m, 2H) |
| 04-53 | 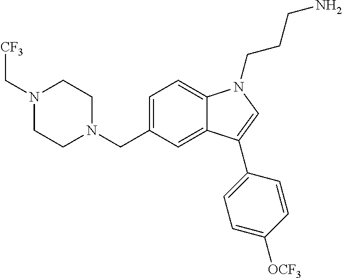 [SMILES: NCCCn1cc(-c2ccc(OC(F)(F)F)cc2)c2cc(CN3CCN(CC(F)(F)F)CC3)ccc21] | 3-[5-[[4-(2,2,2- trifluoroethyl) piperazin-1- yl]methyl]-3-[4- (trifluoromethoxy) phenyl]indol-1- yl]propan-1-amine hydrochloride | Calc'd for C₂₅H₂₉F₆N₄O: 515.2; Found: 515.3 | δ 8.12(8, 1H), 7.84-7.77 (m, 2H), 7.73-7.65 (m, 2H), 7.47-7.42 (m, 1H), 7.36 (br d, J = 8.4 Hz, 2H), 4.52-4.40 (m, 4H), 3.43 (br d, J = 11.9 Hz, 2H), 3.28-309 (m, 6H), 3.00-2.94 (m, 2H), 2.90-2.81 (m, 2H), 2.29-2.21 (m, 2H) |
| 05-40 | 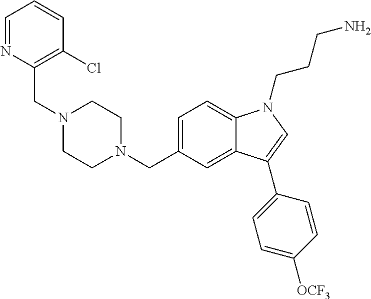 [SMILES: NCCCn1cc(-c2ccc(OC(F)(F)F)cc2)c2cc(CN3CCN(Cc4ncccc4Cl)CC3)ccc21] | 3-(5-((4-((3- chloropyridin-2- yl)methyl) piperazin-1-yl) methyl)-3- (4- (trifluoromethoxy) phenyl)-1H-indol- 1-yl)propan-1- amine hydrochloride | Calc'd for C₂₉H₃₂ClF₃N₅O: 558.2; Found: 558.3 | δ 8.64 (br d, J = 4.0 Hz, 1H), 8.21 (s, 1H), 8.10 (br d, J = 8.4 Hz, 1H), 7.84 (br d, J = 8.4 Hz, 2H), 7.74-7.67 (m, 2H), 7.60-7.50 (m, 2H), 7.37 (br d, J = 7.9 Hz, 2H), 4.72- 4.61 (m, 4H), 4.43 (br t, J = 7.1 Hz, 2H), 3.68 (br s, 8H), 3.02-2.93 (m, 2H), 2.31-2.21 (m, 2H) |
| 05-48 | 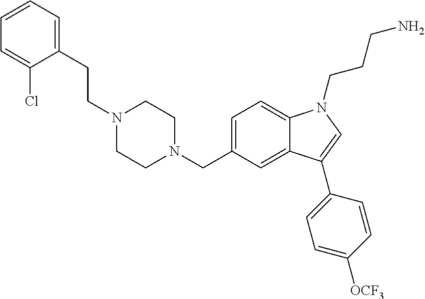 [SMILES: NCCCn1cc(-c2ccc(OC(F)(F)F)cc2)c2cc(CN3CCN(CCc4ccccc4Cl)CC3)ccc21] | 3-(5-((4-(2- chlorophenethyl) piperazin-1- yl)methyl)-3-(4- (trifluoromethoxy) phenyl)-1H-indol- 1-yl)propan-1- amine dihydrochloride | Calc'd for C₃₁H₃₅ClF₃N₄O: 571.2: Found: 571.3 | δ 8.19(8, 1H), 7.84 (s, 1H), 7.81 (s, 1H), 7.70-7.67 (m, 2H), 7.41-7.37 (m, 1H), 7.35-7.30 (m, 2H), 7.30-7.29 (m, 2H), 7.29-7.28 (m, 2H), 4.62 (s, 2H), 4.42 (t, J = 7.2 Hz, 2H), 3.41-3.30 (m, 8H), 3.30-3.29 (m, 2H), 3.28-3.27 (m, 2H), 2.96 (t, J = 7.2 Hz, 2H), 2.28- 2.26 (m, 2H) |
| 05-50 | 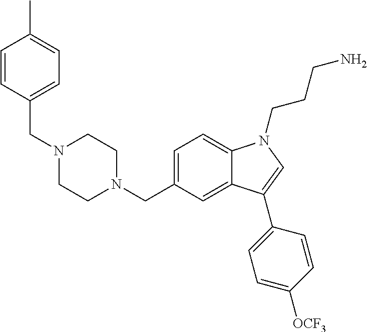 [SMILES: Cc1ccc(CN2CCN(Cc3ccc4c(c3)c(-c3ccc(OC(F)(F)F)cc3)cn4CCCN)CC2)cc1] | 3-(5-((4-(4- methylbenzyl) piperazin-1- yl)methyl)-3-(4- (trifluoromethoxy) phenyl)-1H-indol- 1-yl)propan-1- amine hydrochloride | Calc'd for C₃₁H₃₆F₃N₄O: 537.3: Found: 537.3 | δ 8.18 (s, 1H), 7.83 (br d, J = 8.8 Hz, 2H), 7.71 (s, 1H), 7.67 (d, J = 8.4 Hz, 1H), 7.46 (br d, J = 7.5 Hz, 3H), 7.35 (br d, J = 7.9 Hz, 2H), 7.29 (br d, J = 7.9 Hz, 2H), 4.59 (br s, 2H), 4.46- 4.36 (m, 4H), 3.64 (br s, 8H), 3.01- 2.92 (m, 2H), 2.36 (s, 3H), 2.29-2.20 (m, 2H) |
| 05-53 | 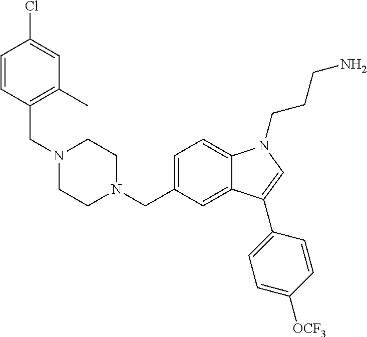 [SMILES: Cc1cc(Cl)ccc1CN1CCN(Cc2ccc3c(c2)c(-c2ccc(OC(F)(F)F)cc2)cn3CCCN)CC1] | 3-(5-((4-(4-chloro- 2- methylbenzyl) piperazin-1- yl)methyl)-3-(4- (trifluoromethoxy) phenyl)-1H-indol- 1-yl)propan-1- amine dihydrochloride | Calc'd for C₃₁H₃₅ClF₃N₄O: 571.2; Found: 571.3 | δ 8.18 (br s, 1H), 7.82 (dd, = 8.49, 1.87 Hz, 2H), 7.72- 7.65 (m, 2H), 7.54 (br s, 1H), 7.49 (br d, J = 8.60 Hz, 1H), 7.36 (br d, J = 7.94 Hz, 3H), 7.29 (br d, J = 8.16 Hz, 1H), 4.60 (br s, 2H), 4.45-4.32 (m, 4H), 3.72-3.4 l (m, 8H), 2.99-2.92 (m, 2H), 2.47 (d, J = 1.54 Hz, 3H), 2.28- 2.19 (m, 2H) |
| 05-54 | 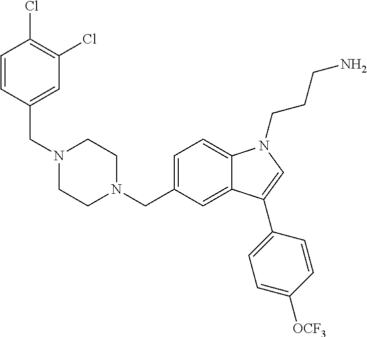 [SMILES: NCCCn1cc(-c2ccc(OC(F)(F)F)cc2)c2cc(CN3CCN(Cc4ccc(Cl)c(Cl)c4)CC3)ccc21] | 3-(5-((4-(3,4- dichlorobenzyl) piperazin-1- yl)methyl)-3-(4- (trifluoromethoxy) phenyl)-1H-indol- 1-yl)propan-1- amine dihydrochloride | Calc'd for C₃₀H₃₂Cl₂F₃N₄O: 591.2; Found: 591.3 | δ 8.18 (s, 1H), 7.84-7.79 (m, 3H), 7.72-7.61 (m, 3 H), 7.53 (dd, J = 8.38, 1.76 Hz, 1H), 7.48 (d, J = 7.28 Hz, 1H), 7.36 (d, J = 8.16 Hz, 2H), 4.60 (s, 2H), 4.45- 4.33 (m, 4H), 3.71- 3.44 (m, 8H), 3.00- 2.92 (m, 2H), 2.29- 2.20 (m, 2H) |
| 05-49 | 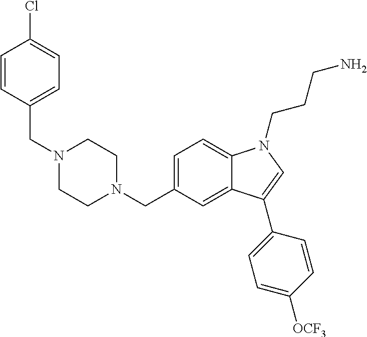 [SMILES: NCCCn1cc(-c2ccc(OC(F)(F)F)cc2)c2cc(CN3CCN(Cc4ccc(Cl)cc4)CC3)ccc21] | 3-(5-((4-(4- chlorobenzyl) piperazin-1-yl) methyl)-3-(4- (trifluoromethoxy) phenyl)-1H-indol- 1-yl)propan-1- amine dihydrochloride | Calc'd for C₃₀H₃₃ClF₃N₄O: 557.2: Found: 557.3 | δ 8.18 (s, 1H), 7.82 (d, J = 8.0 Hz, 2H), 7.68 (m, 1H), 7.66 (m, 2H), 7.49 (d, J = 8.2 Hz, 3H), 7.36 (d, J = 8.0 Hz, 2H), 4.61 (s, 2H), 4.42 (t, J = 6.8 Hz, 4H), 3.74-3.48 (m, 8H), 2.96 (t, J = 7.6 Hz, 2H), 2.28-2.20 (m, 2H) |
| 05-51 | 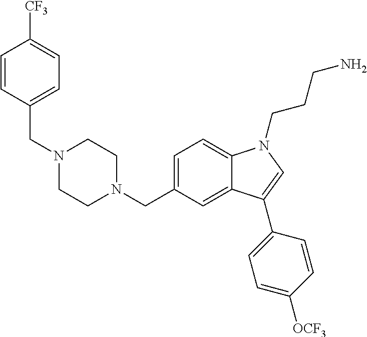 [SMILES: NCCCn1cc(-c2ccc(OC(F)(F)F)cc2)c2cc(CN3CCN(Cc4ccc(C(F)(F)F)cc4)CC3)ccc21] | 3-(3-(4- (trifluoromethoxy) phenyl)-5-((4-(4- (trifluoromethyl) benzyl)piperazin- 1-yl)methyl)-1H- indol-1-yl)propan- 1-amine dihydrochloride | Calc'd for C₃₁H₃₃F₆N₄O: 591.2; Found: 591.3 | δ 8.18 (s, 1H), 7.82 (d, J = 2.0 Hz, 2H), 7.81-7.68 (m, 4H), 7.47 (m, 2H), 7.36 (d, J = 8.2 Hz, 2H), 4.60 (s, 2H), 4.42 (t, J = 6.8 Hz, 4H), 3.74-3.48 (m, 8H), 2.96 (t, J = 7.6 Hz, 2H), 2.28-2.20 (m, 2H) |
| 05-52 | 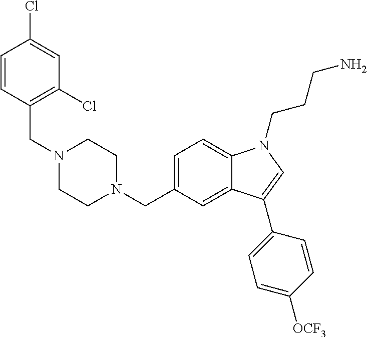 [SMILES: NCCCn1cc(-c2ccc(OC(F)(F)F)cc2)c2cc(CN3CCN(Cc4ccc(Cl)cc4Cl)CC3)ccc21] | 3-(5-((4-(2,4- dichlorobenzyl) piperazin-1- yl)methyl)-3-(4- (trifluoromethoxy) phenyl)-1H-indol- 1-yl)propan-1- amine dihydrochloride | Calc'd for C₃₀H₃₂Cl₂F₃N₄O: 591.2: Found: 591.3 | δ 8.17 (s, 1H), 7.83-7.82 (m, 2H), 7.80-7.76 (m, 3H), 7.69-7.68 (m, 1H), 7.66 (s, 1H), 7.36 (d, J = 8.0 Hz, 2H), 4.58 (d, J = 4.0 Hz, 2H), 4.43- 4.36 (m, 4H), 3.75- 3.35 (m, 8H), 2.96 (t, J = 8.0 Hz, 2H), 2.27-2.20 (m, 2H) |
| 04-23A | 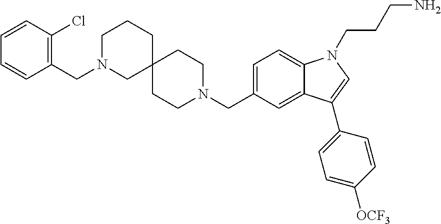 [SMILES: NCCCn1cc(-c2ccc(OC(F)(F)F)cc2)c2cc(CN3CCC4(CCCN(Cc5ccccc5Cl)C4)CC3)ccc21] | 3-[5-[[2-[(2- chlorophenyl) methyl]-2,9- diazaspiro[5.5] undecan-9-yl] methyl]-3-[4- (trifluoromethoxy) phenyl]indol-1- yl]propan-1-amine hydrochloride | Calc'd for C₃₅H₄₁C1F₅N₄O: 625.3: Found: 625.4 | δ 8.12 (s, 1H), 7.83-7.81 (m, 3H), 7.72-7.70 (m, 2H), 7.69-7.46 (m, 4H), 7.36-7.34 (m, 2H), 4.54-4.53 (m, 1H), 4.46-4.41 (m, 3H), 3.52-3.34 (br p, 4H), 3.23-3.08 (m, 6H), 2.42-2.22 (m, 4H), 1.98-1.35 (m, 8H) |
| 04-15A | 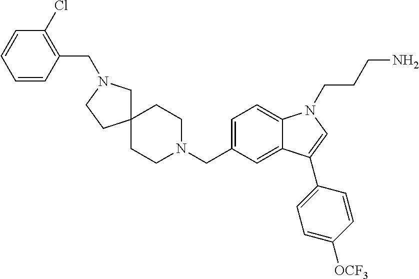 [SMILES: NCCCn1cc(-c2ccc(OC(F)(F)F)cc2)c2cc(CN3CCC4(CC3)CCN(Cc3ccccc3Cl)C4)ccc21] | 3-[5-[[2-[(2- chlorophenyl) methyl]-2,8- diazaspiro[4.5] decan-8-yl] methyl]-3-[4- (trifluoromethoxy) phenyl]indol-1- yl]propan-1-amine hydrochloride | Calc'd for C₃₄H₃₉ClF₃N₄O: 611.3; Found: 611.4 | δ 8.12 (s, 1H), 7.83-7.79 (m, 5H), 7.68-7.66 (m, 1H), 7.49-7.46 (m, 3H), 7.36-7.34 (m, 2H), 4.65-4.60 (p, 2H), 4.46-4.44 (m, 4H), 3.80-3.69 (t, 2H), 3.45 (s, 3H), 3.30- 3.23 (m, 3H), 3.13- 2.98 (m, 2H), 2.28- 2.24 (m, 2H), 2.22- 1.98 (m, 6H) |
| 04-55 | 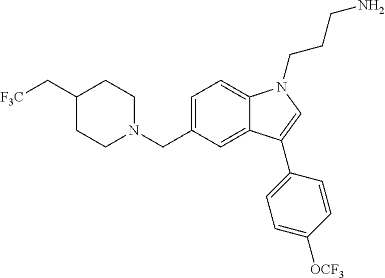 [SMILES: NCCCn1cc(-c2ccc(OC(F)(F)F)cc2)c2cc(CN3CCC(CC(F)(F)F)CC3)ccc21] | 3-(5-((4-(2,2,2- trifluoroethyl) piperidin-1-yl) methyl)- 3-(4- (trifluoromethoxy) phenyl)-1H-indol- 1-yl)propan-1- amine, hydrochloride salt | Calc'd for C₂₇H₃₀F₆N₃O: 514.2; Found: 514.2 | δ 8.12-8.08 (m, 1H), 7.82-7.78 (m, 2H), 7.71 (s, 1H), 7.68 (d, J = 8.38 Hz, 1H), 7.43 (dd, J = 8.38, 1.54 Hz, 1H), 7.37 (d, J = 7.94 Hz, 2H), 4.47- 4.39 (m, 4H), 3.51 (br d, J = 12.79 Hz, 2H), 3.11-2.95 (m, 4H), 2.30-2.17 (m, 4H), 2.06 (br d, J = 13.89 Hz, 3H), 1.68-1.53 (m, 2H) |
| 04-64 | 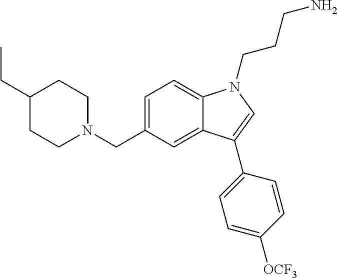 [SMILES: CCC1CCN(Cc2ccc3c(c2)c(-c2ccc(OC(F)(F)F)cc2)cn3CCCN)CC1] | 3-[5-[(4-ethyl-1- piperidyl)methyl]- 3-[4- (trifluoromethoxy) phenyl]indol-1- yl]propan-1- amine, hydrochloride salt | Calc'd for C₂₆H₃₃F₃N₃O: 460.2; Found: 460.3 | δ 8.08 (s, 1H), 7.78-7.81 (m, 2H), 7.70 (s, 1H), 7.67 (d, J = 8.4 Hz 1H), 7.43 (m, 1H), 7.36 (d, J =8.0 Hz 2H), 4.41-4.44 (m, 4H), 3.46-3.49 (m, 2H), 2.95-2.99 (m, 4H), 2.23-2.27 (m, 2H), 1.93-1.96 (m, 2H), 1.42-1.45 (m, 3H), 1.30-1.33 (m, 2H), 0.89-0.93 (m, 3H) |
| 04-58 | 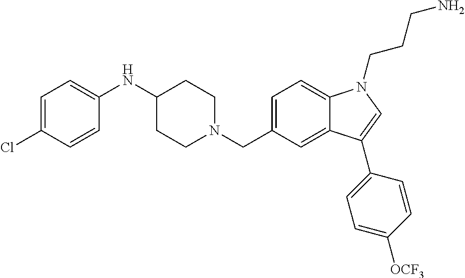 [SMILES: NCCCn1cc(-c2ccc(OC(F)(F)F)cc2)c2cc(CN3CCC(Nc4ccc(Cl)cc4)CC3)ccc21] | 1-((1-(3- aminopropyl)-3- (4- (trifluoromethoxy) phenyl)-1H-indol- 5-yl)methyl)-N-(4- chlorophenyl) piperidin-4-amine, hydrochloride salt | Calc'd for C₃₀H₃₃ClF₃N₄O: 557.2; Found: 557.3 | δ 8.09 (s, 1H), 7.79 (dd, J = 6.8, 2.0 Hz 2H), 7.64-7.68 (m, 2H), 7.35 (m, 1H), 7.32-7.34 (m, 4H), 7.05-7.07 (m, 2H), 4.50 (s, 2H), 4.38- 4.41 (m, 2H), 3.58 (m, 1H), 3.55 (m, 2H), 3.11-3.14 (m, 2H), 2.92-2.96 (m, 2H), 2.20-2.24 (m, 4H), 1.89-1.93 (m, 2H) |
| 04-56 | 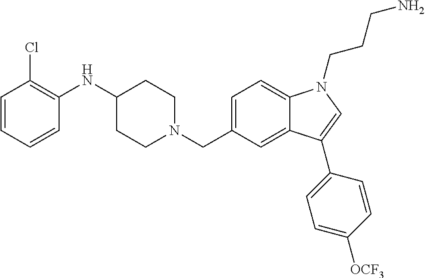 [SMILES: NCCCn1cc(-c2ccc(OC(F)(F)F)cc2)c2cc(CN3CCC(Nc4ccccc4Cl)CC3)ccc21] | 1-((1-(3- aminopropyl)-3- (4- (trifluoromethoxy) phenyl)-1H-indol- 5-yl)methyl)-N-(2- chlorophenyl) piperidin-4-amine, hydrochloride salt | Calc'd for C₃₀H₃₃ClF₃N₄O: 557.2; Found: 557.1 | δ 8.52 (brs, 1H), 7.91 (s, 1H), 7.75 (d, J = 6.8 Hz, 2H), 7.73 (s, 1H), 7.51-7.57 (m, 1H), 7.32-7.35 (m, 3H), 7.19 (m, 1H), 7.09- 7.17 (m, 1H), 6.75 (m, 1H), 6.57 (m, 1H), 4.38 (t, J = 6.4 Hz, 2H), 3.89 (s, 2H), 3.48-3.49 (m, 1H), 3.07 (m, 2H), 2.88-2.92 (m, 2H), 2.51 (m, 2H), 2.18-2.22 (m, 2H), 2.06-2.09 (m, 2 H), |
| | | | | 1.58-1.61 (m, 2H) |
| 04-57 | 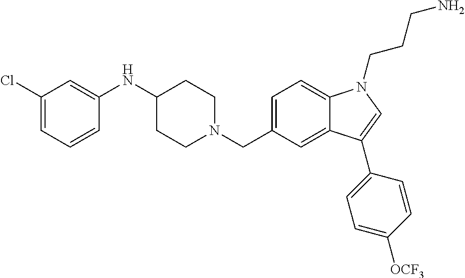 [SMILES: NCCCn1cc(-c2ccc(OC(F)(F)F)cc2)c2cc(CN3CCC(Nc4cccc(Cl)c4)CC3)ccc21] | l-((1-(3- aminopropyl)-3- (4- (trifluoromethoxy) phenyl)-1H-indol- 5-yl)methyl)-N-(3- chlorophenyl) piperidin-4-amine, hydrochloride salt | Calc'd for C₃₀H₃₃ClF₃N₄O: 557.2; Found: 557.3 | δ 8.48-8.47 (m, 1H), 8.04-8.02 (m, 1H), 7.77-7.75 (m, 2H), 7.66-7.61 (m, 2H), 7.40-7.37 (m, 3H), 7.02-7.00 (m, 1H), 6.61-6.60 (m, 1H), 6.55-6.53 (m, 2H), 4.41-4.39 (m, 2H), 4.37-4.32 (m, 2H), 3.53-3.51 (m, 1H), 3.37-3.34 (m, 2H), 3.04-3.00 (m, 2H), 2.96-2.94 (m, 2H), 2.24-2.20 (m, 4H), 1.70-1.68 (m, 2H) |
| 04-60 | 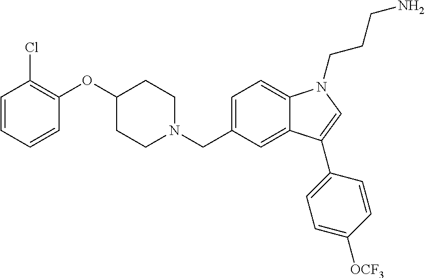 [SMILES: NCCCn1cc(-c2ccc(OC(F)(F)F)cc2)c2cc(CN3CCC(Oc4ccccc4Cl)CC3)ccc21] | 3-(5-((4-(2- chlorophenoxy) piperidin-1- yl)methyl)-3-(4- (trifluoromethoxy) phenyl)-1H-indol- 1-yl)propan-1- amine, hydrochloride salt | Calc'd for C₃₀H₃₂ClF₃N₃O₂: 558.2; Found: 558.3 | δ 8.12 (s, 1H), 7.83-7.77 (m, 2H), 7.72-7.66 (m, 2H), 7.45 (dd, J = 8.60, 1.54 Hz, 1H), 7.36 (dt, J = 8.05, 1.60 Hz, 3H), 7.29- 7.23 (m, 1H), 7.15 (dd, J = 8.38, 1.32 Hz, 1H), 6.97 (td, J = 7.66, 1.43 Hz, 1H), 4.50 (s, 2H), 4.43 (t, J = 6.95 Hz, 2H), 3.57- 3.33 (m, 1H), 3.36- 3.32 (m, 1H), 3.32- 3.31 (m, 1H), 3.31- |
| | | | | 3.31 (m, 4H), 3.29- |
| | | | | 3.27 (m, 1H), 3.00- |
| | | | | 2.94 (m, 2H), 2.30- |
| | | | | 2.05 (m, 6H) |
| 04-62 | 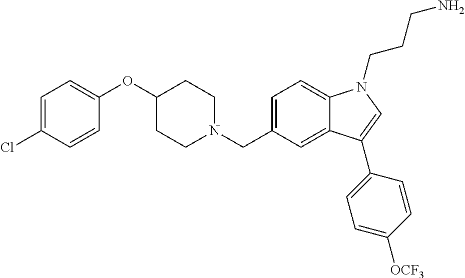 [SMILES: NCCCn1cc(-c2ccc(OC(F)(F)F)cc2)c2cc(CN3CCC(Oc4ccc(Cl)cc4)CC3)ccc21] | 3-(5-((4-(4- chlorophenoxy) piperidin-1- yl)methyl)-3-(4- (trifluoromethoxy) phenyl)-1H-indol- 1-yl)propan-1- amine dihydrochloride | Calc'd for C₃₀H₃₂ClF₃N₃O₂: 558.2; Found: 558.3 | δ 8.14-8.10 (m, 1H), 7.83-7.77 (m, 2H), 7.72-7.67(m, 2H), 7.48-7.41 (m, 1H), 7.38 (d, J = 8.82 Hz, 2H), 7.32- 7.24 (m, 2H), 7.05- 6.93 (m, 2H), 4.75 (br s, 1H), 4.51 (s, 2H), 4.44 (t, J = 6.95 Hz, 2H), 3.43- 3.33 (m, 4H), 3.03- 2.95 (m, 2H), 2.42- 2.03 (m, 6H) |
| 04-61 | 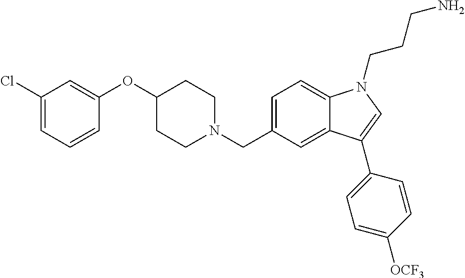 [SMILES: NCCCn1cc(-c2ccc(OC(F)(F)F)cc2)c2cc(CN3CCC(Oc4cccc(Cl)c4)CC3)ccc21] | 3-(5-((4-(3- chlorophenoxy) piperidin-1- yl)methyl)-3-(4- (trifluoromethoxy) phenyl)-1H-indol- 1-yl)propan-1- amine dihydrochloride | Calc'd for C₃₀H₃₂ClF₃N₃O₂: 558.2; Found: 558.3 | δ 8.11 (s, 1H), 7.80-7.77 (m, 2H), 7.69-7.68 (m, 2H), 7.43-7.42 (m, 1H), 7.37-7.35 (m, 2H), 7.27-7.25 (m, 1H), 7.07-7.06 (m, 1H), 6.99-6.97 (m, 2H), 4.78-4.76 (m, 2H), 4.49-4.43 (m, 2H), 4.41-4.40 (m, 2H), 3.59-3.56 (m, 3H), 2.98-2.97 (m, 2H), 2.34-2.19 (m, 6H) |
| 08-6 | 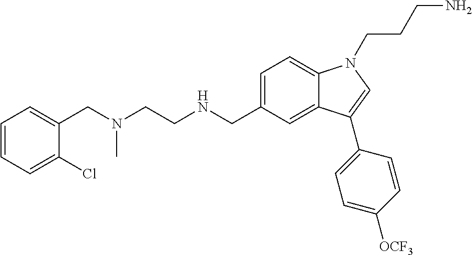 [SMILES: CN(CCNCc1ccc2c(c1)c(-c1ccc(OC(F)(F)F)cc1)cn2CCCN)Cc1ccccc1Cl] | N1-((1-(3- aminopropyl)-3- (4- (trifluoromethoxy) phenyl)-1H-indol- 5-yl)methyl)-N2- (2-chlorobenzyl)- N2-methylethane- 1,2-diamine dihydrochloride | Calc'd for C₂₉H₃₃ClF₃N₄O: 545.2; Found: 545.3 | δ 8.15 (s, 1H), 7.86-7.78 (m, 3H), 7.70 (s, 1H), 7.65 (br d, J = 8.4 Hz, 1H), 7.58-7.54 (m, 1H), 7.47 (br dd, J = 6.2, 14.1 Hz, 3H), 7.36 (br d, J = 7.9 Hz, 2H), 4.69- 4.58 (m, 2H), 4.48- 4.38 (m, 4H), 3.68 (br s, 4H), 3.03- 2.89 (m, 5H), 2.28- 2.21 (m, 2H) |
| 08-7 | 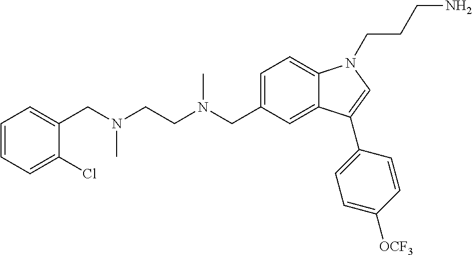 [SMILES: CN(CCN(C)Cc1ccccc1Cl)Cc1ccc2c(c1)c(-c1ccc(OC(F)(F)F)cc1)cn2CCCN] | Nl-((l-(3- aminopropyl)-3- (4- (trifluoromethoxy) phenyl)-lH-indol- 5-yl)methyl)-N2- (2-chlorobenzyl)- N1,N2- dimethylethane- 1,2-diamine dihydrochloride | Calc'd for C₃₀H₃₅ClF₃N₄O: 559.2; Found: 559.3 | δ 8.22 (s, 1H), 7.86 (br d, J = 8.4 Hz, 2H), 7.73 (s, 2H), 7.69 (br d, J = 8.4 Hz, 1H), 7.55-7.46 (m, 3H), 7.44-7.33 (m, 3H), 4.60 (br s, 4H), 4.43 (br t, J = 6.6 Hz, 2H), 3.88 (br s, 4H), 3.01- 2.94 (m, 2H), 2.89 (br d, J = 4.4 Hz, 6H), 2.31-2.22 (m, 2H) |
| 08-5 | 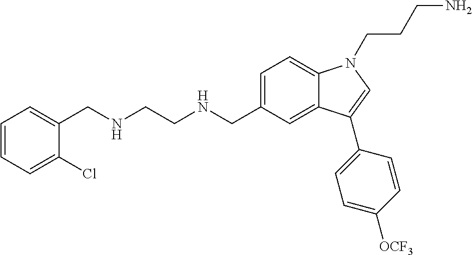 [SMILES: NCCCn1cc(-c2ccc(OC(F)(F)F)cc2)c2cc(CNCCNCc3ccccc3Cl)ccc21] | Nl-((1-(3- aminopropyl)-3- (4- (trifluoromethoxy) phenyl)-1H-indol- 5-yl)methyl)-N2- (2- chlorobenzyl) ethane-1-2-diamine dihydrochloride | Calc'd for C₂₈H₃₁ClF₃N₄O: 531.2; Found: 531.3 | δ 8.17 (d, J = 1.3 Hz, 1H), 7.83 (d, J = 7.7 Hz, 2H), 7.73-7.64 (m, 3H), 7.56-7.53 (m, 1H), 7.50-7.41 (m, 3H), 7.36 (dd, J = 0.9, 8.8 Hz, 2H), 4.48- 4.39 (m, 6H),3.61- 3.53 (m, 4H), 2.99- 2.94 (m, 2H), 2.29- 2.21 (m, 2H) |

### Example 23: Synthesis of 4-[1-(3-aminopropyl)-5-[[4-[(2,6-dichlorophenyl)methyl]piperazin-1-yl]methyl]indol-3-yl]benzonitrile (06-5)

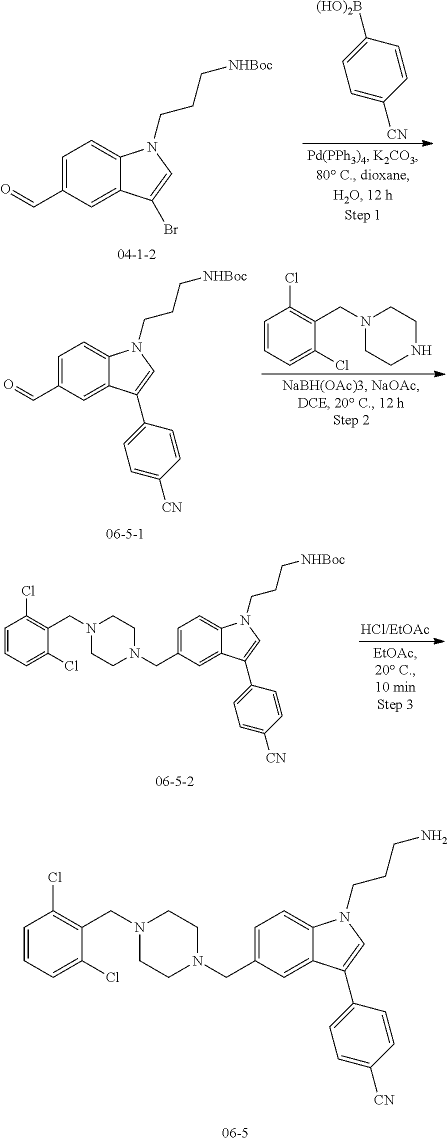 [SMILES: CC(C)(C)OC(=O)NCCCn1cc(Br)c2cc(C=O)ccc21 ; N#Cc1ccc(B(O)O)cc1 ; CC(C)(C)OC(=O)NCCCn1cc(-c2ccc(C#N)cc2)c2cc(C=O)ccc21 ; Clc1cccc(Cl)c1CN1CCNCC1 ; CC(C)(C)OC(=O)NCCCn1cc(-c2ccc(C#N)cc2)c2cc(CN3CCN(Cc4c(Cl)cccc4Cl)CC3)ccc21 ; N#Cc1ccc(-c2cn(CCCN)c3ccc(CN4CCN(Cc5c(Cl)cccc5Cl)CC4)cc23)cc1]

### Step 1: tert-butyl N-[3-[3-(4-cyanophenyl)-5-formyl-indol-1-yl]propyl]carbamate (06-5-1)

To a solution of tert-butyl N-[3-(3-bromo-5-formyl-indol-1-yl)propyl]carbamate (1.00 g, 2.62 mmol, 1.0 eq) in dioxane (10 mL) and H₂O (1 mL) was added (4-cyanophenyl)boronic acid (577.5 mg, 3.93 mmol, 1.5 eq), K₂CO₃ (724.2 mg, 5.24 mmol, 2.0 eq) and Pd(PPh₃)₄(151.4 mg, 131.0 μmol, 0.05 eq). The mixture was stirred at 80° C. for 12 h under N₂. Water (50 mL) was added and the mixture was extracted with EtOAc (20 mL*3). The combined organic layers were dried over anhydrous Na₂SO₄, filtered, and concentrated in vacuum. The residue was purified by column chromatography (SiO₂) to give compound 06-5-1 (630 mg, 1.56 mmol, 60% yield).

### Step 2: tert-butyl N-[3-[3-(4-cyanophenyl)-5-[[4-[(2,6-dichlorophenyl)methyl]piperazin-1-yl]methyl]indol-1-yl]propyl]carbamate (06-5-2)

To a solution of 06-5-1 (630.0 mg, 1.56 mmol, 1.0 eq) in DCE (10 mL) was added 1-[(2,6-dichlorophenyl)methyl]piperazine (483.2 mg, 1.72 mmol, 1.1 eq, HCl salt), NaOAc (639.8 mg, 7.80 mmol, 5.0 eq) and the mixture was stirred at 20° C. for 1 h. Then NaBH(OAc)₃ (661.2 mg, 3.12 mmol, 2.0 eq) was added and stirred at 20° C. for 11 h. The reaction mixture was poured into water (100 mL) and extracted with DCM (50 mL*3). The combined organic layers were dried over anhydrous Na₂SO₄, filtered, and concentrated in vacuum. The residue was purified by column chromatography (SiO₂) to give compound 06-5-2 (820 mg, 1.30 mmol, 83% yield). 250 mg of compound 06-5-2 was further purified by acidic prep-HPLC (TFA) to give 120 mg of TFA salt of the compound after lyophilization.

### Step 3: 4-[1-(3-aminopropyl)-5-[[4-[(2,6-dichlorophenyl)methyl]piperazin-1-yl]methyl]indol-3-yl]benzonitrile (06-5)

To a solution of 06-5-2 (120.0 mg, 160.7 μmol, 1.0 eq, TFA salt) in EtOAc (1 mL) was added HCl/EtOAc (4 M, 1.0 mL, 24.9 eq) at 20° C. dropwise. After the addition, the reaction mixture was stirred at 20° C. for 10 min. During the reaction a solid precipitated. The reaction mixture was filtered and the filter cake was washed with DCM (5 mL*3). Then the solid was collected and dried under reduced pressure to give compound 06-5 (70.4 mg, 122.3 μmol, 76% yield, HCl salt). M+H⁺=532.3 (LCMS). ¹H NMR (MeOD, 400 MHz): δ 8.30 (s, 1H), 7.97 (d, J=8.4 Hz, 2H), 7.89 (s, 1H), 7.78 (d, J=8.4 Hz, 2H), 7.71 (d, J=8.4 Hz, 1H), 7.62-7.46 (m, 4H), 4.72 (s, 2H), 4.65 (s, 2H), 4.45 (br t, J=7.1 Hz, 2H), 3.81 (br s, 4H), 3.75-3.56 (m, 4H), 3.03-2.93 (m, 2H), 2.31-2.20 (m, 2H).

The following compounds are synthesized from int. 04-1-2 in similar procedures as described above for the preparation of 06-5.

| Comp | | | Mass | NMR (MeOD, 400 |
|---|---|---|---|---|
| ID | Structure | Chemical Name | (M + H⁺) | MHz) |
| 06- 13- int4 | 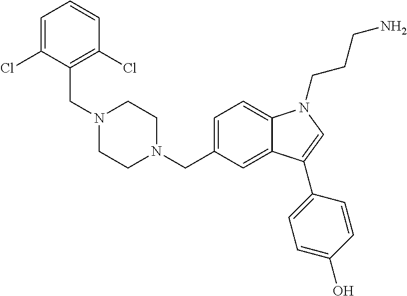 [SMILES: NCCCn1cc(-c2ccc(O)cc2)c2cc(CN3CCN(Cc4c(Cl)cccc4Cl)CC3)ccc21] | 3-(5-((4-(2,6- dichlorobenzyl) piperazin-1-yl) methyl)-3- (p-tolyl)-1H-indol-1- yl)propan-1-amine dihydrochloride | Calc'd for C₂₉H₃₃Cl₂N₄O: 523.2; Found: 523.2 | δ 8.09 (s, 1H), 7.62 (d, J = 8.4 Hz, 1H), 7.55-7.46 (m, 7H), 6.87 (d,J= 8.8 Hz, 2H), 4.64 (s, 2H), 4.58 (s, 2H), 4.38 (t, J = 6.8 Hz, 2H), 3.72-3.67 (m, 8H), 2.94 (t, J = 8 Hz, 2H), 2.24-2.20 (m, 2H) |
| 06-11 | 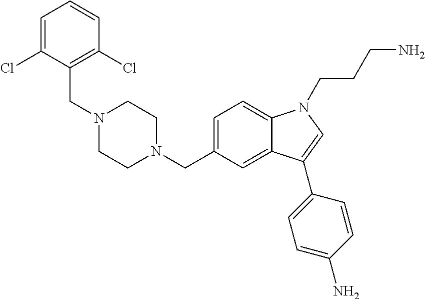 [SMILES: NCCCn1cc(-c2ccc(N)cc2)c2cc(CN3CCN(Cc4c(Cl)cccc4Cl)CC3)ccc21] | 4-(1-(3- aminopropyl)-5-((4- (2,6- dichlorobenzyl) piperazin-1-yl) methyl)-1H-indol- 3-yl)aniline dihydrochloride | Calc'd for C₂₉H₃₄Cl₂N₃: 522.2; Found: 522.3 | δ 8.30 (s, 1H), 7.97 (d, J = 8.8 Hz, 2H), 7.81 (s, 1H), 7.69 (d, J = 8.8 Hz, 1H), 7.59-7.55 (m, 2H), 7.53-7.47 (m, 4H), 4.72 (s, 2H), 4.64 (s, 2H), 4.44 (br t, J = 7.1 Hz, 2H), 3.82 (br s, 4H), 3.71 (br s, 4H), 3.01-2.94 (m, 2H), 2.30-2.22 (m, 2H) |
| 06-7- int4 | 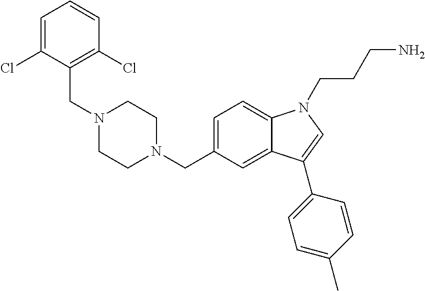 [SMILES: Cc1ccc(-c2cn(CCCN)c3ccc(CN4CCN(Cc5c(Cl)cccc5Cl)CC4)cc23)cc1] | 3-(5-((4-(2,6- dichlorobenzyl) piperazin-1-yl) methyl)-3- (p-tolyl)-1H-indol-1- yl)propan-1-amine dihydrochloride | Calc'd for C₃₀H₃₅Cl₂N₄: 521.2; Found: 521.2 | δ 8.14 (s, 1H), 7.65- 7.53 (m, 6H), 7.47 (m, 2H), 7.27 (d, J = 8 Hz, 2H), 4.58 (s, 2H), 4.55 (s, 2H), 4.42-4.39 (m, 2H), 3.65-3.53 (m, 8H), 2.95 (t, J = 8 Hz, 2H), 2.38 (s, 3H), 2.27-2.22 (m, 2H) |
| 06-3 int4 | 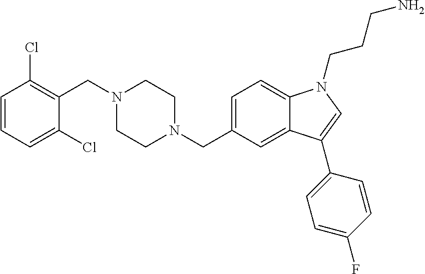 [SMILES: NCCCn1cc(-c2ccc(F)cc2)c2cc(CN3CCN(Cc4c(Cl)cccc4Cl)CC3)ccc21] | 3-(5-((4-(2,6- dichlorobenzyl) piperazin-1-yl) methyl)-3- (4-methoxyphenyl)- 1H-indol-1- yl)propan-1-amine diformate | Calc'd for C₂₉H₃₂Cl₂FN₄: 525.2; Found: 525.3 | δ 8.53 (s, 1H), 7.91 (s, 1H), 7.69-7.66 (m, 2H), 7.54-7.53 (m, 2H), 7.40 (d, J = 8 Hz, 2H), 7.34- 7.22 (m, 2H), 7.21- 7.19 (m, 2H), 4.39 (t, J = 6.8 Hz, 2H), 3.96 (s, 2H), 3.84 (s, 2H), 2.94 (t, J = 6.8 Hz, 2H), 2.81- 2.74 (m, 8H), 2.27- 2.19 (m, 2H) |
| 06-9- int4 | 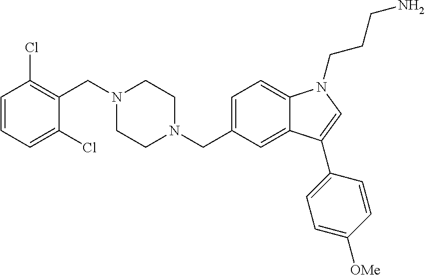 [SMILES: COc1ccc(-c2cn(CCCN)c3ccc(CN4CCN(Cc5c(Cl)cccc5Cl)CC4)cc23)cc1] | 3-(5-((4-(2,6- dichlorobenzyl) piperazin-1-yl) methyl)-3- (4-methoxyphenyl)- 1H-indol-1- yl)propan-1-amine diformate | Calc'd for C₃₀H₃₅Cl₂N₄O: 537.2; Found: 537.3 | δ 8.50 (s, 2H), 7.97 (s, 1H), 7.60-7.55 (m, 3H), 7.50(s, 1H), 7.41-7.39 (m, 2H), 7.30-7.28 (m, 2H), 7.03-7.01 (m, 2H), 4.38 (t, J = 7.2 Hz, 2H), 4.20 (s, 2H), 3.86 (s, 2H), 3.34 (s, 2H), 3.33 (s, 2H), 3.05-2.96 (m, 4H), 2.94-2.92 (m, 2H), 2.80-2.75 (m, 3H), 2.27-2.20 (m, 2H) |

### Example 24: Synthesis of 2-[1-(3-aminopropyl)-5-[[4-[(2,6-dichlorophenyl)methyl]piperazin-1-yl]methyl]indol-3-yl]benzonitrile (06-20)

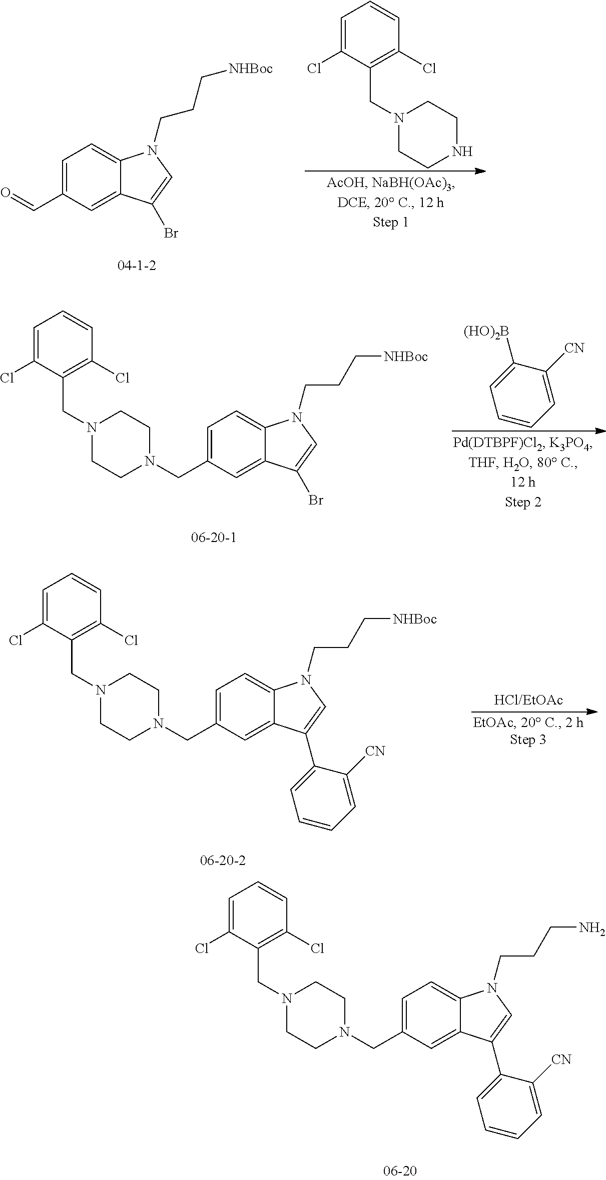 [SMILES: CC(C)(C)OC(=O)NCCCn1cc(Br)c2cc(C=O)ccc21 ; Clc1cccc(Cl)c1CN1CCNCC1 ; CC(C)(C)OC(=O)NCCCn1cc(Br)c2cc(CN3CCN(Cc4c(Cl)cccc4Cl)CC3)ccc21 ; N#Cc1ccccc1B(O)O ; CC(C)(C)OC(=O)NCCCn1cc(-c2ccccc2C#N)c2cc(CN3CCN(Cc4c(Cl)cccc4Cl)CC3)ccc21 ; N#Cc1ccccc1-c1cn(CCCN)c2ccc(CN3CCN(Cc4c(Cl)cccc4Cl)CC3)cc12]

### Step 1: tert-butyl N-[3-[3-bromo-5-[[4-[(2,6-dichlorophenyl)methyl]piperazin-1-yl]methyl]indol-1-yl]propyl]carbamate (06-20-1)

To a stirred solution of 1-[(2,6-dichlorophenyl)methyl]piperazine (3.00 g, 10.65 mmol, 1.0 eq, HCl salt) and tert-butyl N-[3-(3-bromo-5-formyl-indol-1-yl)propyl]carbamate (4.47 g, 11.7 mmol, 1.1 eq) in DCE (50.00 mL) was added NaOAc (4.37 g, 53.3 mmol, 5.0 eq), then the reaction mixture was stirred at 20° C. for 2 h. NaBH(OAc)₃ (6.77 g, 31.9 mmol, 3.0 eq) was added to the mixture in portions. The mixture was stirred at 20° C. for 10 h, poured to water (100 mL), and extracted with DCM (100 mL*2). The combined organic layers were washed with H₂O (50 mL*2), dried over anhydrous Na₂SO₄, filtered and concentrated under reduced pressure to give a residue. The residue was purified by column chromatography (SiO₂) to give compound 06-20-1 (3.60 g, 5.13 mmol, 48% yield). M+H⁺=611.2 (LCMS).

### Step 2: tert-butyl N-[3-[3-(2-cyanophenyl)-5-[[4-[(2,6-dichlorophenyl)methyl]piperazin-1-yl]methyl]indol-1-yl]propyl]carbamate (06-20-2)

A mixture of 06-20-1 (100.0 mg, 163.8 μmol, 1.0 eq), (2-cyanophenyl)boronic acid (21.7 mg, 147.4 μmol, 0.9 eq), K₃PO₄ (69.5 mg, 327.6 μmol, 2.0 eq) and [1,1′-Bis(di-tert-butylphosphino)ferrocene]dichloropalladium(II) (10.7 mg, 16.4 μmol, 0.1 eq) in THF (5 mL) and H₂O (1 mL) was degassed and then heated to 80° C. for 12 h under N₂. The reaction mixture was added to water (20 mL), and extracted with EtOAc (10 mL*3). The combined organic layer was washed with brine (20 mL), dried over anhydrous Na₂SO₄, then filtered and the filtrate was concentrated under reduced pressure. The residue was purified by prep-TLC to give compound 06-20-2 (50.0 mg, 48% yield). M+H⁺=632.3 (LCMS).

### Step 3: 2-[1-(3-aminopropyl)-5-[[4-[(2,6-dichlorophenyl)methyl]piperazin-1-yl]methyl]indol-3-yl]benzonitrile (06-20)

To a solution of 06-20-2 (50.0 mg, 79.0 μmol, 1.0 eq) in EtOAc (1.0 mL) was added HCl/EtOAc (4 M, 1.0 mL, 50.6 eq), then the reaction mixture was stirred at 20° C. for 2 h. During the reaction a solid precipitated. The reaction mixture was filtered. The filter cake was washed with DCM (5 mL*2). Then the solid was collected and dried under reduced pressure to give compound 06-20 (30.4 mg, 50.2 μmol, 64% yield, HCl salt). M+H⁺=532.3 (LCMS). ¹H NMR (MeOD, 400 MHz): δ 7.96 (s, 1H), 7.87-7.72 (m, 5H), 7.56-7.47 (m, 4H), 7.47-7.42 (m, 1H), 4.57 (s, 2H), 4.55-4.44 (m, 4H), 3.58 (br s, 8H), 3.02-2.95 (m, 2H), 2.30-2.21 (m, 2H).

### Example 25: Synthesis of 3-[3-[2-(aminomethyl)phenyl]-5-[[4-[(2,6-dichlorophenyl)methyl]piperazin-1-yl]methyl]indol-1-yl]propan-1-amine (06-21)

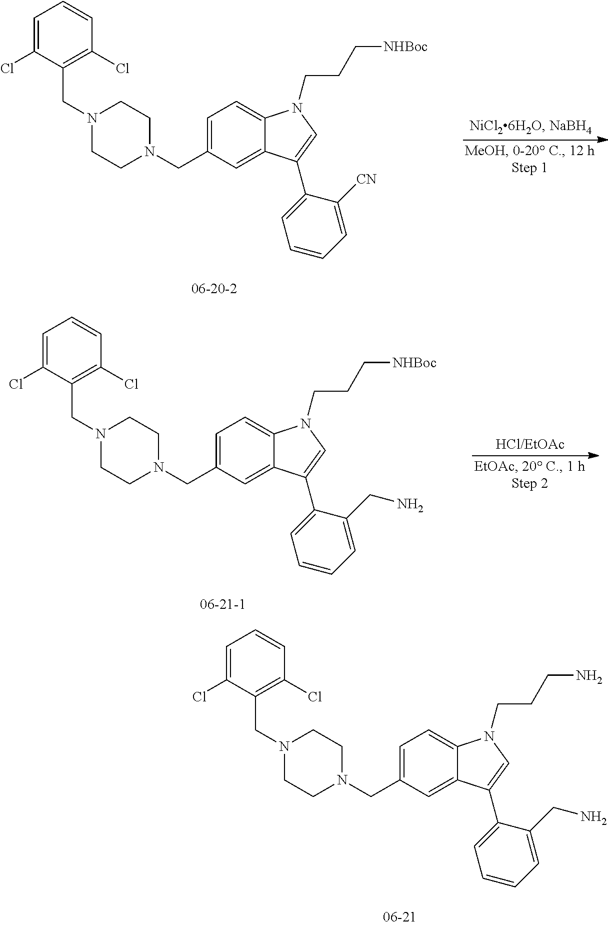 [SMILES: CC(C)(C)OC(=O)NCCCn1cc(-c2ccccc2C#N)c2cc(CN3CCN(Cc4c(Cl)cccc4Cl)CC3)ccc21 ; CC(C)(C)OC(=O)NCCCn1cc(-c2ccccc2CN)c2cc(CN3CCN(Cc4c(Cl)cccc4Cl)CC3)ccc21 ; NCCCn1cc(-c2ccccc2CN)c2cc(CN3CCN(Cc4c(Cl)cccc4Cl)CC3)ccc21]

### Step 1: tert-butyl N-[3-[3-[2-(aminomethyl)phenyl]-5-[[4-[(2,6-dichlorophenyl)methyl]piperazin-1-yl]methyl]indol-1-yl]propyl]carbamate (06-21-1)

To a stirred solution of tert-butyl N-[3-[3-(2-cyanophenyl)-5-[[4-[(2,6-dichlorophenyl)methyl]piperazin-1-yl]methyl]indol-1-yl]propyl]carbamate (300.0 mg, 474.2 μmol, 1.0 eq) in MeOH (10 mL) was added NiCl₂.6H₂O (112.7 mg, 474.2 μmol, 1.0 eq) at 20° C., then the mixture was cooled to 0° C. and NaBH₄ (90.1 mg, 2.38 mmol, 5.0 eq) was added in portions. After 12 h at 20° C., the reaction mixture was diluted with water (30 mL), and filtered through a pad of Celite. The filter cake was washed with DCM (20 mL×5). The organic layer was washed with brine (50 mL), dried over anhydrous Na₂SO₄, filtered and the filtrate was concentrated under reduced pressure. The residue was purified by acidic prep-HPLC to give compound 06-21-1 (100.0 mg, 27% yield, TFA salt).

### Step 2: 3-[3-[2-(aminomethyl)phenyl]-5-[[4-[(2,6-dichlorophenyl)methyl]piperazin-1-yl]methyl]indol-1-yl]propan-1-amine (06-21)

To a solution of 06-21-1 (100.0 mg, 1.0 eq, TFA) in EtOAc (1 mL) was added HCl/EtOAc (4 M, 2.12 mL, 63.7 eq) at 20° C. Then the reaction mixture was stirred at 20° C. for 1 h. The reaction mixture was filtered and the cake was washed with DCM (5 mL*3). After, the solid was dissolved in water (5 mL) and lyophilized to give compound 06-21 (45.0 mg, 76.6 μmol, 58% yield, HCl salt). M+H⁺=536.3 (LCMS). ¹H NMR (MeOD, 400 MHz): δ 7.74-7.70 (m, 2H), 7.63 (dd, J=3.6, 5.2 Hz, 1H), 7.61-7.57 (m, 3H), 7.55-7.48 (m, 5H), 4.83 (s, 2H), 4.60 (s, 2H), 4.47 (br t, J=6.9 Hz, 2H), 4.24 (s, 2H), 3.95 (br s, 4H), 3.75 (br s, 4H), 3.05-2.97 (m, 2H), 2.29 (quin, J=7.3 Hz, 2H).

The following compounds are synthesized according to similar procedures as described above for the preparation of 06-20 and 06-21.

| Comp | | | Mass | NMR (MeOD, 400 |
|---|---|---|---|---|
| ID | Structure | Chemical Name | (M + H⁺) | MHz) |
| 06-18 | 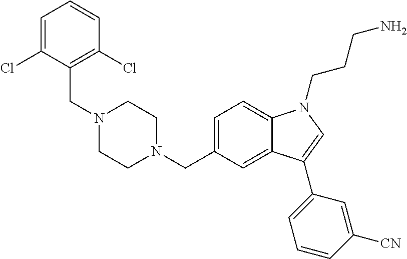 [SMILES: N#Cc1cccc(-c2cn(CCCN)c3ccc(CN4CCN(Cc5c(Cl)cccc5Cl)CC4)cc23)c1] | 3-(5-((4-(2,6- dichlorobenzyl) piperazin-1- yl)methyl)-3-(p- tolyl)-1H-indol-1- yl)propan-1-amine dihydrochloride | Calc'd for C₃₀H₃₂Cl₂N₅: 532.2; Found: 532.2 | δ 8.14 (s, 1H), 8.07- 8.02 (m, 2H), 7.80 (s, 1H), 7.69 (d, J = 8.6 Hz, 1H), 7.65- 7.61 (m, 2H), 7.50- 7.42 (m, 3H), 7.39- 7.32 (m, 1H), 4.52 (s, 2H), 4.43 (t, J = 6.9 Hz, 2H), 4.18 (br s, 2H), 3.58- 3.31 (m, 8H), 2.99- 2.94 (m, 2H), 2.24 (quin, J = 7.5 Hz, 2H) |
| 06-19 | 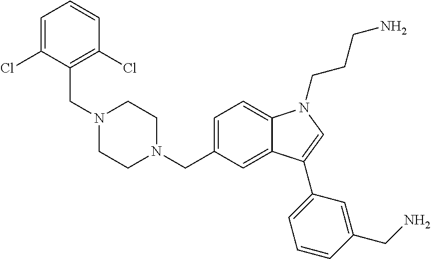 [SMILES: NCCCn1cc(-c2cccc(CN)c2)c2cc(CN3CCN(Cc4c(Cl)cccc4Cl)CC3)ccc21] | 3-(3-(3- (aminomethyl) phenyl)-5-((4-(2,6- dichlorobenzyl) piperazin-1- yl)methyl)-1H- indol-1-yl)propan- 1-amine trihydrochloride | Calc'd for C₃₀H₃₆Cl₂N₅: 536.2; Found: 536.3 | δ 8.47 (s, 1H), 8.08 (s, 1H), 7.79-7.73 (m, 2H), 7.65 (br d, J = 8.4 Hz, 1H), 7.53- 7.48 (m, 3H), 7.45- 7.39 (m, 2H), 7.35 (br d, J = 7.9 Hz, 1H), 4.59 (s, 2H), 4.47-4.37 (m, 4H), 4.26 (s, 2H), 3.51 (br s, 8H), 3.01- 2.94 (m, 2H), 2.30- 2.21 (m, 2H) |
| 06-24 | 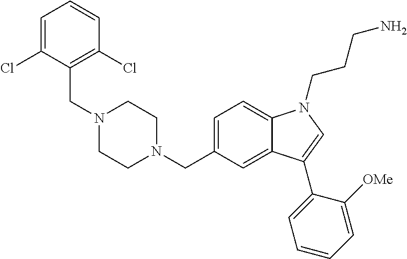 [SMILES: COc1ccccc1-c1cn(CCCN)c2ccc(CN3CCN(Cc4c(Cl)cccc4Cl)CC3)cc12] | 3-(5-((4-(2,6- dichlorobenzyl) piperazin-1- yl)methyl)-3-(2- methoxyphenyl)- 1H-indol-1- yl)propan-1-amine dihydrochloride | Calc'd for C₃₀H₃₅Cl₂N₄O: 537.2: Found: 537.3 | δ 7.83 (s, 1H), 7.57- 7.46 (m, 5H), 7.42- 7.36 (m, 2H), 7.21- 7.16 (m, 1H), 7.00 (d, J = 7.9 Hz, 1H), 6.97-6.91 (m, 1H), 4.63 (s, 2H), 4.51 (s, 2H), 4.32 (br t, J = 6.7 Hz, 2H), 3.70 (br d, J = 11.2 Hz, 3H), 3.58 (br d, J = 18.3 Hz, 4H), 3.21 (td, J = 1.6, 3.3 Hz, 4H), 2.90-2.83 (m, 2H), 2.19-2.09 (m, 2H) |
| 06-23 | 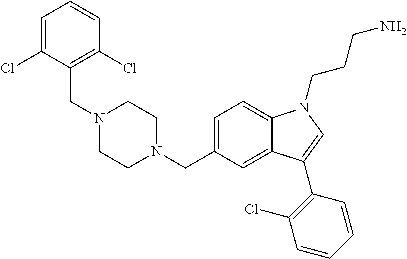 [SMILES: NCCCn1cc(-c2ccccc2Cl)c2cc(CN3CCN(Cc4c(Cl)cccc4Cl)CC3)ccc21] | 3-(3-(2- chlorophenyl)-5- ((4-(2,6- dichlorobenzyl) piperazin-1- yl)methyl)-1H- indol-1-yl)propan- 1-amine dihydrochloride | Calc'd for C₂₉H₃₂Cl₃N₄: 541.2; Found: 541.2 | δ 7.70 (d, J = 1.1 Hz, 1H), 7.59 (d, J = 8.6 Hz, 1H), 7.56-7.50 (m, 2H), 7.46-7.36 (m, 5H), 7.30 (dt, J = 1.3, 7.5 Hz, 1H), 7.24-7.18 (m, 1H), 4.57 (s, 2H), 4.48 (s, 2H), 4.35 (t, J = 6.9 Hz, 2H), 3.69- 3.47 (m, 8H), 2.92- 2.83 (m, 2H), 2.20- 2.11 (m, 2H) |
| 06-22 | 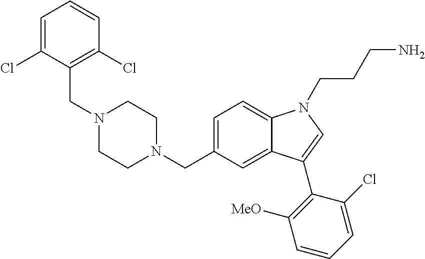 [SMILES: COc1cccc(Cl)c1-c1cn(CCCN)c2ccc(CN3CCN(Cc4c(Cl)cccc4Cl)CC3)cc12] | 3-(3-(2-chloro-6- methoxyphenyl)-5- ((4-(2,6- dichlorobenzyl) piperazin-1- yl)methyl)-1H- indol-1-yl)propan- 1-amine dihydrochloride | Calc'd for C₃₀H₃₄Cl₃N₄O: 571.2; Found: 571.3 | δ 7.66 (br d, J = 8.8 Hz, 1H), 7.55-7.50 (m, 2H), 7.48-7.40 (m, 4H), 7.35-7.31 (m, 1H), 7.13 (d, J = 7.9 Hz, 1H), 7.06 (d, J = 8.4 Hz, 1H), 4.51 (s, 4H), 4.43 (br s, 2H), 3.72 (s, 3H), 3.66-3.42 (m, 8H), 2.96 (br t, J = 7.7 Hz, 2H), 2.28- 2.21 (m, 2H) |

### Example 26: Synthesis of 3-[5-[[8-[(2-chlorophenyl)methyl]-3,8-diazabicyclo[3.2.1]octan-3-yl]methyl]-3-(4-methoxyphenyl)indol-1-yl]propan-1-amine (06-34)

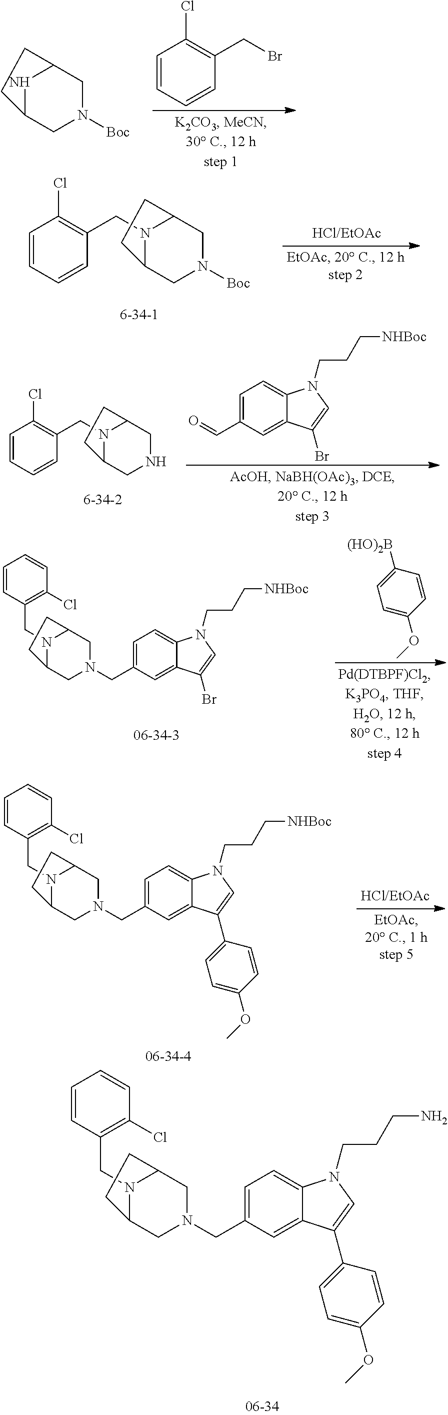

### Step 1: tert-butyl 8-[(2-chlorophenyl)methyl]-3,8-diazabicyclo[3.2.1]octane-3-carboxylate (06-34-1)

To a solution of 1-(bromomethyl)-2-chloro-benzene (1.16 g, 5.65 mmol, 734.2 μL, 1.2 eq) and tert-butyl 3,8-diazabicyclo[3.2.1]octane-3-carboxylate (1.00 g, 4.71 mmol, 1.0 eq) in MeCN (30 mL) was added K₂CO₃ (1.30 g, 9.42 mmol, 2.0 eq). The mixture was stirred at 30° C. for 12 h under N₂, filtered, and the filtrate was concentrated in vacuum. The residue was purified by column chromatography (SiO₂) to give compound 06-34-1 (1.40 g, 77% yield,). ¹H NMR (CDCl₃, 400 MHz): δ 7.67 (br d, J=7.50 Hz, 1H), 7.34 (br d, J=7.94 Hz, 1H), 7.30-7.24 (m, 1H), 7.22-7.15 (m, 1H), 3.77 (br d, J=11.91 Hz, 1H), 3.67-3.57 (m, 3H), 3.22-3.09 (m, 3H), 3.05 (br d, J=11.91 Hz, 1H), 2.08-1.95 (m, 2H), 1.69 (br dd, J=15.00, 7.94 Hz, 2H), 1.47 (s, 9H).

### Step 2: 8-[(2-chlorophenyl)methyl]-3,8-diazabicyclo[3.2.1]octane (06-34-2)

To a solution of 06-34-1 (1.40 g, 1.0 eq) in EtOAc (3 mL) was added HCl/EtOAc (4 M, 20 mL, 19.2 eq). The mixture was stirred at 20° C. for 12 h. The reaction mixture was filtered and collected solid was dissolved in H₂O (80 mL). The aqueous solution was adjusted to pH 9 with solid NaOH, and extracted with dichloromethane (30 mL*5). The combined organic layers were washed with brine (60 mL), dried over anhydrous Na₂SO₄, filtered and the filtrate was concentrated in vacuum to give compound 06-34-2 (860.0 mg, 3.60 mmol, 87% yield).

### Step 3: tert-butyl N-[3-[3-bromo-5-[[8-[(2-chlorophenyl)methyl]-3,8-diazabicyclo[3.2.1]octan-3-yl]methyl]indol-1-yl]propyl]carbamate (06-34-3)

To a solution of 06-34-2 (400.0 mg, 1.69 mmol, 1.0 eq) and tert-butyl N-[3-(3-bromo-5-formyl-indol-1-yl)propyl]carbamate (773.2 mg, 2.03 mmol, 1.2 eq) in DCE (10 mL) was added AcOH (101.5 mg, 1.69 mmol, 96.7 μL, 1.0 eq). The mixture was stirred at 20° C. for 2 h. Then NaBH(OAc)₃ (716.4 mg, 3.38 mmol, 2.0 eq) was added to the mixture portionwise. The resulting mixture was stirred at 20° C. for another 10 h under N₂ and poured into sat. NaHCO₃ (80 mL). The mixture was extracted with DCM (30 mL*3). The combined organic layers were washed with brine (50 mL), dried over anhydrous Na₂SO₄, filtered and the filtrate was concentrated in vacuum. The residue was purified by column chromatography (SiO₂) to give compound 06-34-3 (580.0 mg, crude). ¹H NMR (CDCl₃, 400 MHz): δ 7.74 (br s, 1H), 7.57-7.43 (m, 1H), 7.40-7.23 (m, 4H), 7.22-7.00 (m, 3H), 5.40-5.15 (m, 1H), 4.54 (br s, 1H), 4.15 (br s, 2H), 3.76-3.51 (m, 4H), 3.26-3.01 (m, 4H), 2.72-2.37 (m, 4H), 2.08-1.87 (m, 6H), 1.52-1.34 (m, 9H).

### Step 4: tert-butyl N-[3-[5-[[8-[(2-chlorophenyl)methyl]-3,8-diazabicyclo[3.2.1]octan-3-yl]methyl]-3-(4-methoxyphenyl)indol-1-yl]propyl]carbamate (06-34-4)

To a solution of 06-34-3 (170.0 mg, 282.4 μmol, 1.0 eq) and (4-methoxyphenyl)boronic acid (38.6 mg, 254.2 μmol, 0.9 eq) in H₂O (1 mL) and THF (5 mL) was added K₃PO₄ (119.9 mg, 564.8 μmol, 2.0 eq) and [1,1′-Bis(di-tert-butylphosphino)ferrocene]dichloropalladium(II) (18.4 mg, 28.2 μmol, 0.1 eq). The mixture was stirred at 80° C. for 12 h under N₂. The reaction mixture was poured into H₂O (80 mL), and extracted with EtOAc (30 mL*3). The combined organic layers were washed with brine (50 mL), dried over anhydrous Na₂SO₄, filtered and the filtrate was concentrated in vacuum. The residue was purified by prep-TLC (SiO₂) to give compound 06-34-4 (40.0 mg, 21% yield). ¹H NMR (CDCl₃, 400 MHz): δ 7.79 (s, 1H), 7.72 (br d, J=7.50 Hz, 1H), 7.58 (br d, J=8.38 Hz, 2H), 7.33-7.25 (m, 5H), 7.20-7.15 (m, 2H), 7.01 (br d, J=8.38 Hz, 2H), 4.20 (br t, J=6.84 Hz, 2H), 3.88 (s, 3H), 3.61 (br d, J=3.97 Hz, 4H), 3.22-3.10 (m, 4H), 2.64 (br d, J=8.82 Hz, 2H), 2.41 (br d, J=10.14 Hz, 2H), 2.13-2.07 (m, 2H), 2.00-1.88 (m, 4H), 1.45 (s, 9H).

### Step 5: 3-[5-[[8-[(2-chlorophenyl)methyl]-3,8-diazabicyclo[3.2.1]octan-3-yl]methyl]-3-(4-methoxyphenyl)indol-1-yl]propan-1-amine (06-34)

To a solution of 06-34-4 (40.0 mg, 1.0 eq) in EtOAc (1 mL) was added HCl/EtOAc (4 M, 1.0 mL, 62.9 eq). The mixture was stirred at 20° C. for 1 h. The reaction mixture was filtered and the solid was washed with DCM (5 mL*3), collected and dried in vacuum to give compound 06-34 (19.8 mg, 33.5 μmol, 53% yield, HCl salt). M+H⁺=529.3 (LCMS). ¹H NMR (MeOD, 400 MHz): δ 8.05 (br s, 1H), 7.83 (br d, J=5.73 Hz, 1H), 7.59 (br d, J=8.38 Hz, 3H), 7.55-7.49 (m, 2H), 7.48-7.37 (m, 3H), 7.01 (br d, J=8.82 Hz, 2H), 4.51-4.27 (m, 6H), 4.16 (br s, 2H), 3.89-3.79 (m, 3H), 3.38 (br d, J=19.85 Hz, 4H), 2.99-2.90 (m, 2H), 2.56 (br s, 2H), 2.40 (br s, 2H), 2.27-2.18 (m, 2H).

### Example 27: Synthesis of 3-[5-[[8-[(2-chlorophenyl)methyl]-3,8-diazabicyclo[3.2.1]octan-3-yl]methyl]-3-(4-ethylphenyl)indol-1-yl]propan-1-amine (06-35)

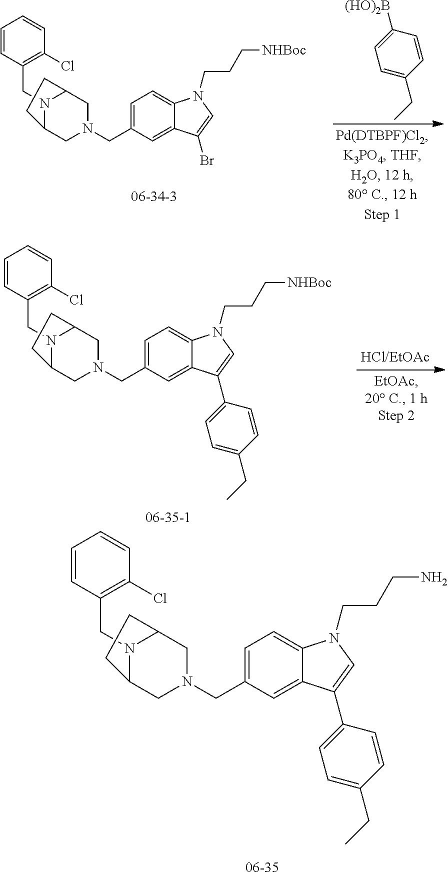 [SMILES: CC(C)(C)OC(=O)NCCCn1cc(Br)c2cc(CN3CC4CCC(C3)N4Cc3ccccc3Cl)ccc21 ; CCc1ccc(B(O)O)cc1 ; CCc1ccc(-c2cn(CCCNC(=O)OC(C)(C)C)c3ccc(CN4CC5CCC(C4)N5Cc4ccccc4Cl)cc23)cc1 ; CCc1ccc(-c2cn(CCCN)c3ccc(CN4CC5CCC(C4)N5Cc4ccccc4Cl)cc23)cc1]

Compound 06-35 was synthesized according to a similar procedure as described in step 4 and step 5 of the preparation of 06-34.

### tert-butyl N-[3-[5-[[8-[(2-chlorophenyl)methyl]-3,8-diazabicyclo[3.2.1]octan-3-yl]methyl]-3-(4-ethylphenyl)indol-1-yl]propyl]carbamate (06-35-1)

¹H NMR (CDCl₃, 400 MHz): δ 7.80 (s, 1H), 7.69 (br d, J=7.1 Hz, 1H), 7.55 (br d, J=7.9 Hz, 2H), 7.30-7.22 (m, 6H), 7.20 (s, 1H), 7.15-7.10 (m, 1H), 4.17 (br t, J=6.8 Hz, 2H), 3.58 (br d, J=4.0 Hz, 4H), 3.17-3.05 (m, 4H), 2.68 (q, J=7.8 Hz, 2H), 2.61 (br d, J=9.3 Hz, 2H), 2.38 (br d, J=9.3 Hz, 2H), 2.08-2.03 (m, 2H), 1.90 (br s, 4H), 1.41 (s, 9H), 1.27 (t, J=7.5 Hz, 3H).

### 3-[5-[[8-[(2-chlorophenyl)methyl]-3,8-diazabicyclo[3.2.1]octan-3-yl]methyl]-3-(4-ethylphenyl)indol-1-yl]propan-1-amine (06-35)

M+H⁺=527.2 (LCMS). ¹H NMR (MeOD, 400 MHz): δ 8.05 (br s, 1H), 7.81 (br d, J=5.3 Hz, 1H), 7.66-7.49 (m, 5H), 7.49-7.38 (m, 3H), 7.28 (br d, J=7.9 Hz, 2H), 4.49-4.25 (m, 6H), 4.13 (br s, 2H), 3.58-3.34 (m, 4H), 2.98-2.91 (m, 2H), 2.68 (q, J=7.8 Hz, 2H), 2.54 (br s, 2H), 2.37 (br s, 2H), 2.27-2.19 (m, 2H), 1.27 (t, J=7.7 Hz, 3H).

### Example 28: Synthesis of [2-[1-(3-aminopropyl)-5-[[4-[(2-chlorophenyl)methyl]piperazin-1-yl]methyl]indol-3-yl]phenyl]methanol (06-25)

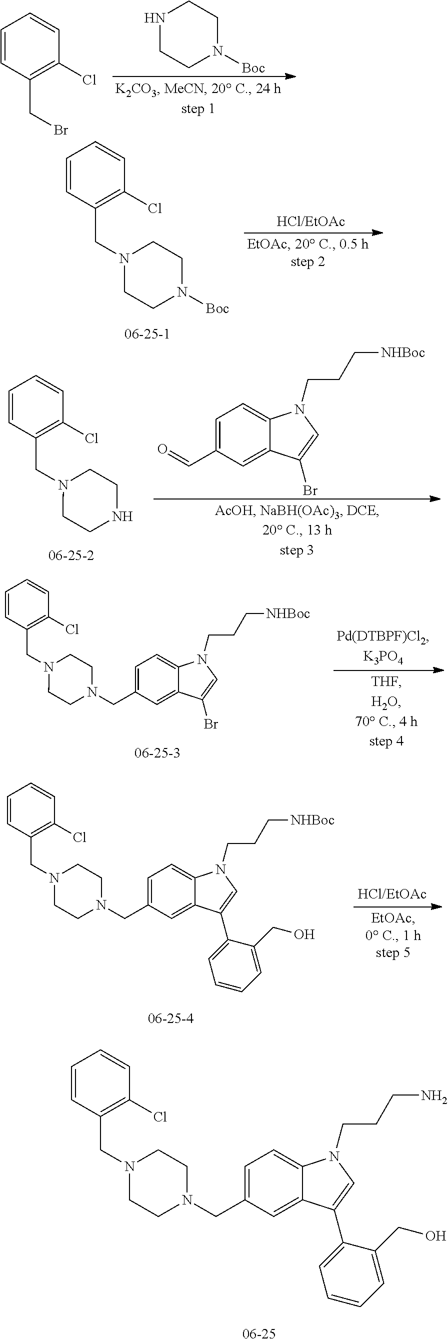

### Step 1: tert-butyl 4-[(2-chlorophenyl)methyl]piperazine-1-carboxylate (06-25-1)

To a solution of 1-(bromomethyl)-2-chloro-benzene (10.0 g, 48.7 mmol, 6.33 mL, 1.0 eq) in MeCN (150 mL) was added tert-butyl piperazine-1-carboxylate (10.9 g, 58.9 mmol, 1.2 eq) and K₂CO₃ (12.9 g, 93.9 mmol, 1.9 eq). The mixture was stirred at 20° C. for 24 h, and filtered. The filtrate was diluted with water (100 mL) and extracted with DCM (200 mL). The combined organic layers were washed with brine (300 mL), dried over anhydrous Na₂SO₄, filtered and concentrated under reduced pressure to give compound 06-25-1 (15.0 g, 89% yield). ¹H NMR (CDCl3, 400 MHz): δ 7.39 (dd, J=7.47, 1.44 Hz, 1H) 7.28 (dd, J=7.72, 1.32 Hz, 1H) 7.16-7.20 (m, 1H) 7.11-7.15 (m, 1H) 7.10 (br d, J=1.76 Hz, 1H) 3.55 (s, 2H) 3.33-3.40 (m, 4H) 2.35-2.42 (m, 4H) 1.39 (s, 9H).

### Step 2: 1-[(2-chlorophenyl)methyl]piperazine (06-25-2)

To a solution of 06-25-1 (15.0 g, 1.0 eq) in EtOAc (10 mL) was added HCl/EtOAc (4 M, 20.0 mL, 1.7 eq). The mixture was stirred at 20° C. for 30 min. Then the reaction mixture was diluted with aqueous of NaOH (1 M) to adjust pH to 9 and extracted with EtOAc (50 mL). The combined organic layers were washed with brine (50 mL), dried over anhydrous Na₂SO₄, filtered and concentrated under reduced pressure to give compound 06-25-2 (9.20 g, 39.3 mmol, 81% yield). ¹H NMR (CDCl3, 400 MHz): δ 7.48 (dd, J=7.50, 1.54 Hz, 1H) 7.34 (dd, J=7.83, 1.43 Hz, 1H) 7.15-7.27 (m, 2H) 3.61 (s, 2H) 2.89-2.93 (m, 4H) 2.49 (br s, 4H) 1.66 (br s, 1H).

### Step 3: tert-butyl N-[3-[3-bromo-5-[[4-[(2-chlorophenyl)methyl]piperazin-1-yl]methyl]indol-1-yl]propyl]carbamate (06-25-3)

To a solution of 06-25-2 (4.00 g, 18.9 mmol, 1.0 eq) in DCE (80 mL) was added AcOH (1.14 g, 18.9 mmol, 1.09 mL, 1.0 eq) and tert-butyl N-[3-(3-bromo-5-formyl-indol-1-yl)propyl]carbamate (8.00 g, 20.9 mmol, 1.11 eq). The mixture was stirred at 20° C. for 2 h. Then NaBH(OAc)₃ (4.02 g, 18.9 mmol, 1.0 eq) was added to the solution. The mixture was stirred at 20° C. for 11 h, diluted with saturated aqueous of NaHCO₃ (20 mL) to adjust pH to 8 and extracted with DCM (100 mL). The combined organic layers were washed with brine (200 mL), dried over anhydrous Na₂SO₄, filtered and concentrated under reduced pressure to give a residue. The residue was purified by column chromatography (SiO₂) to give compound 06-25-3 (3.00 g, 5.21 mmol, 27% yield). M+H⁺=576.0 (LCMS).

### Step 4: tert-butyl N-[3-[5-[[4-[(2-chlorophenyl)methyl]piperazin-1-yl]methyl]-3-[2-(hydroxymethyl)phenyl]indol-1-yl]propyl]carbamate (06-25-4)

To a solution of 06-25-3 (200.0 mg, 347.2 μmol, 1.0 eq) in THF (8 mL) and H₂O (2 mL) was added[2-(hydroxymethyl)phenyl]boronic acid (52.8 mg, 347.2 μmol, 1.0 eq), [1,1′-Bis(di-tert-butylphosphino)ferrocene]dichloropalladium(II) (22.6 mg, 34.7 μmol, 0.1 eq) and K₃PO₄ (147.4 mg, 694.5 μmol, 2.0 eq). The suspension was degassed and purged with N₂ for 3 times. The mixture was stirred at 70° C. for 4 h, cooled to rt, diluted with water (15 mL) and extracted with EtOAc (15 mL). The combined organic layers were washed with brine (20 mL), dried over anhydrous Na₂SO₄, filtered and concentrated under reduced pressure to give a residue. The residue was purified by prep-TLC (SiO₂) to give compound 06-25-4 (80.0 mg).

### Step 5: [2-[1-(3-aminopropyl)-5-[[4-[(2-chlorophenyl)methyl]piperazin-1-yl]methyl]indol-3-yl]phenyl]methanol (06-25)

To a solution of 06-25-4 (80.0 mg, 132.6 μmol, 1.0 eq) in EtOAc (1 mL) was added HCl/EtOAc (4 M, 3.00 mL, 90.5 eq). The mixture was stirred at 0° C. for 1 h. The solid was collected and dried to give compound 06-25 (33.9 mg, 55.4 μmol, 42% yield, HCl salt). M+H⁺=503.3 (LCMS). ¹H NMR (DMSO-d₆, 400 MHz): δ 8.15 (br s, 2H), 7.80-7.70 (m, 3H), 7.66-7.60 (m, 1H), 7.59-7.38 (m, 5H), 7.36 (dd, J=3.4, 5.6 Hz, 1H), 4.50 (s, 2H), 4.48-4.31 (m, 4H), 3.63-3.17 (m, 8H), 2.82 (br d, J=6.5 Hz, 2H), 2.61-2.54 (m, 2H), 2.25-2.05 (m, 2H).

The following compounds are synthesized according to similar procedures as described above for the preparation of 06-25.

| Comp | | | Mass | NMR (MeOD, 400 |
|---|---|---|---|---|
| ID | Structure | Chemical Name | (M + H⁺) | MHz) |
| 06-26 | 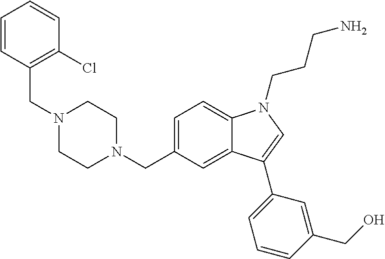 [SMILES: NCCCn1cc(-c2cccc(CO)c2)c2cc(CN3CCN(Cc4ccccc4Cl)CC3)ccc21] | (3-(1-(3- aminopropyl)-5- ((4- (2-chlorobenzyl) piperazin-1-yl) methyl)-1H-indol- 3-yl)phenyl) methanol dihydrochloride | Calc'd for C₃₀H₃₆ ClN₄O: 502.2; Found: 503.3 | ¹H NMR (DMSO- d₆, 400 MHz): δ 8.28-8.06 (m, 4H), 7.85 (s, 1H), 7.74- 7.63 (m, 2H), 7.59 (br d, J = 7.5 Hz, 1H), 7.53 (br d, J = 7.1 Hz, 1H), 7.49- 7.29 (m, 4H), 7.19 (br d, J = 7.5 Hz, 1H), 5.06-4.69 (m, 4H), 4.62-4.44 (m, 4H), 4.36 (br t, J = 6.4 Hz, 2H), 3.67- 3.32 (m, 7H), 2.75 (br d, J = 5.7 Hz, 2H), 2.15-2.02 (m, 2H) |
| 06-36 | 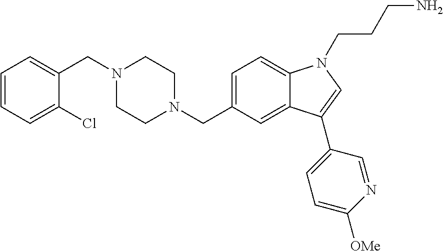 [SMILES: COc1ccc(-c2cn(CCCN)c3ccc(CN4CCN(Cc5ccccc5Cl)CC4)cc23)cn1] | 3-(5-((4-(2- chlorobenzyl) piperazin-1-yl) methyl)-3-(6- methoxypyridin- 3-yl)-1H-indol-1- yl)propan-1-amine dihydrochloride | Calc'd for C₂₉H₃₅ ClN₅O: 504.2; Found: 504.3 | δ 8.96-8.90 (m, 1H), 8.79 (d, J = 1.8 Hz, 1H), 8.36 (s, 1H), 7.98 (s, 1H), 7.79 (dd, J = 1.7, 7.4 Hz, 1H), 7.70 (dd, J = 4.7, 8.9 Hz, 2H), 7.59-7.44 (m, 4H), 4.65 (br d, J = 5.7 Hz, 4H), 4.44 (t, J = 6.9 Hz, 2H), 4.27 (s, 3H), 3.81 (br s, 4H), 3.72 (br s, 4H), 3.02-2.94 (m, 2H), 2.30-2.18 (m, 2H) |

### Example 29

| Comp | | | Mass | NMR (MeOD, 400 |
|---|---|---|---|---|
| ID | Structure | Chemical Name | (M + H⁺) | MHz) |
| 06-45 | 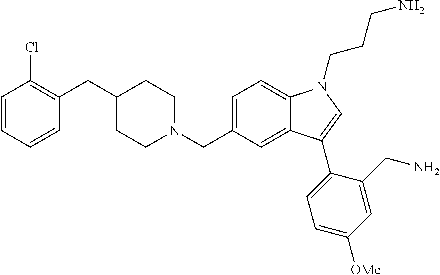 [SMILES: COc1ccc(-c2cn(CCCN)c3ccc(CN4CCC(Cc5ccccc5Cl)CC4)cc23)c(CN)c1] | 3-(3-(2- (aminomethyl)-4- (trifluoromethoxy) phenyl)-5-((4-(2- chlorobenzyl) piperidin-1-yl) methyl)-1H-indol- 1-yl)propan- 1-amine dihydrochloride | Calc'd for C₃₂H₃₇Cl F₃N₄O: 585.3; Found: 585.3 | δ7.70 (d, J = 8.6 Hz, 1H), 7.66-7.60 (m, 4H), 7.47-7.41 (m, 2H), 7.36 (dd, J = 1.8, 7.3 Hz, 1H), 7.28-7.17 (m, 3H), 4.47 (br t, J = 6.6 Hz, 2H), 4.35 (s, 2H), 4.26 (s, 2H), 3.44 (br d, J = 11.5 Hz, 2H), 3.04-2.91 (m, 4H), 2.75 (d, J = 6.8 Hz, 2H), 2.31 (br d, J = 7.5 Hz, 2H), 1.99 (br s, 1H), 1.86 (br d, J = 13.7 Hz, 2H), 1.72- 1.54 (m, 2H) |
| 06-46 | 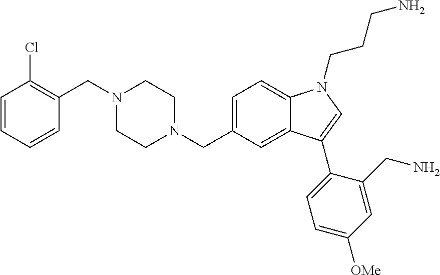 [SMILES: COc1ccc(-c2cn(CCCN)c3ccc(CN4CCN(Cc5ccccc5Cl)CC4)cc23)c(CN)c1] | 3-(3-(2- (aminomethyl)-4- methoxyphenyl)- 5-((4-(2- chlorobenzyl) piperazin-1- yl)methyl)-1H- indol-1-yl) propan-1-amine dihydrochloride | Calc'd for C₃₁H₃₉Cl N₅O: 532.3; Found: 532.4 | δ 7.83 (dd, J = 1.5, 7.5 Hz, 1H), 7.71- 7.66 (m, 2H), 7.60- 7.55 (m, 1H), 7.54- 7.40 (m, 5H), 7.21 (d, J = 2.4 Hz, 1H), 7.06 (dd, J = 2.6, 8.4 Hz, 1H), 4.68 (s, 2H), 4.58 (s, 2H), 4.44 (br t, J = 6.9 Hz 2H), 4.18 (s, 2H), 3.90- 3.88 (m, 3H), 3.82 (br s, 4H), 3.72 (br s, 4H), 3.03-2.95 (m, 2H), 2.28 (quin, J = 7.3 Hz, 2H) |
| 06-47 | 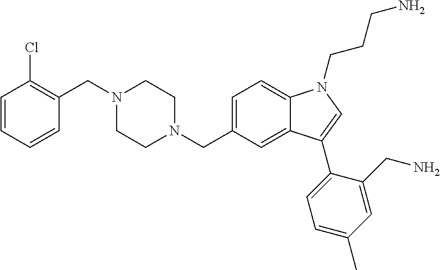 [SMILES: Cc1ccc(-c2cn(CCCN)c3ccc(CN4CCN(Cc5ccccc5Cl)CC4)cc23)c(CN)c1] | 3-(3-(2- (aminomethyl)-4- methylphenyl)-5- ((4-(2- chlorobenzyl) piperazin- 1-yl)methyl)- 1H-indol-1-yl) propan-1-amine dihydrochloride | Calc'd for C₃₁H₃₉Cl N₅: 516.3; Found: 516.4 | δ 7.83 (m, 1H), 7.70 (d, J = 7.5 Hz, 2H), 7.59-7.53 (m, 2H), 7.53-7.43 (m, 4H), 7.41-7.36 (m, 1H), 7.31 (d, J = 7.9 Hz, 1H), 4.67 (s, 2H), 4.58 (s, 2H), 4.45 (t, J = 6.8 Hz, 2H), 4.19 (s, 2H), 3.82 (br s, 4H), 3.72 (br s, 4H), 3.02-2.98 (m, 2H), 2.46 (s, 3H), 2.28 (quin, J = 7.3 Hz, 2H) |
| 06-61 | 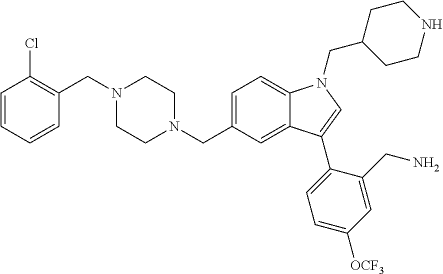 [SMILES: NCc1cc(OC(F)(F)F)ccc1-c1cn(CC2CCNCC2)c2ccc(CN3CCN(Cc4ccccc4Cl)CC3)cc12] | (2-(5-((4-(2- chlorobenzyl)piper azin-1-yl)methyl)- 1-(piperidin-4- ylmethyl)-1H- indol-3-yl)-5- (trifluoromethoxy) phenyl) methanamine dihydrochloride | Calc'd for C₃₄H₄₀Cl F₃N₅O: 626.3; Found: 626.4 | δ 7.83-7.79 (m, 1H), 7.73-7.70 (m, 2H), 7.67-7.64 (m, 1H), 7.61-7.56 (m, 3H), 7.54-7.42 (m, 4H), 4.65 (s, 4H), 4.27 (s, 4H), 3.85-3.58 (m, 8H), 3.45-3.38 (m, 2H), 3.03-2.92 (m, 2H), 2.40-2.28 (m, 1H), 1.90-1.80 (m, 2H), 1.68-1.55 (m, 2H) |
| 06-53 | 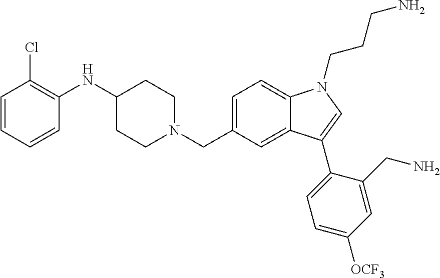 [SMILES: NCCCn1cc(-c2ccc(OC(F)(F)F)cc2CN)c2cc(CN3CCC(Nc4ccccc4Cl)CC3)ccc21] | 1-((3-(2- (aminomethyl)-4- (trifluoromethoxy) phenyl)-1-(3- aminopropyl)-1H- indol-5-yl)methyl)- N-(2- chlorophenyl) piperidin-4-amine dihydrochloride | Calc'd for C₃₁H₃₆Cl F₃N₅O: 586.2; Found: 586.3 | δ 7.71-7.75 (m, 2 H) 6.63-6.68 (m, 3 H) 7.48 (m, 2 H) 7.32 (m, 1 H) 7.22-7.30 (m, 1 H) 7.00 (m, 1 H) 6.80 (m, 1 H) 4.47-4.53 (m, 2 H) 4.49 (s, 2 H) 4.29 (s, 2 H) 3.79 (m, 1 H) 3.54-3.57 (m, 2 H) 3.21 (m, 2 H) 3.01- 3.05 (m, 2 H) 2.27- 2.33 (m, 4 H) 1.90- 1.94 (m, 2 H) |
| 06-55 | 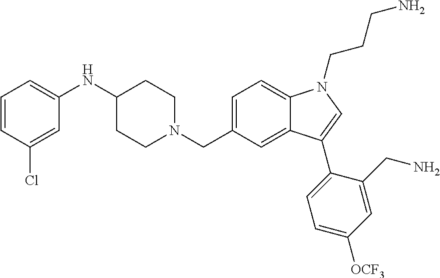 [SMILES: NCCCn1cc(-c2ccc(OC(F)(F)F)cc2CN)c2cc(CN3CCC(Nc4cccc(Cl)c4)CC3)ccc21] | l-((3-(2- (aminomethyl)-4- (trifluoromethoxy) phenyl)-1-(3- aminopropyl)-1H- indol-5-yl)methyl)- N-(3- chlorophenyl) piperidin-4-amine dihydrochloride | Calc'd for C₃₁H₃₆Cl F₃N₅O: 586.2; Found: 586.3 | δ 7.73-7.61 (m, 5H), 7.49-7.47 (m, 2H), 7.25-7.23 (m, 1H), 7.03-7.02 (m, 1H), 6.93-6.76 (m, 2H), 4.49-4.43 (m, 4H), 4.28 (s, 2H), 3.73- 3.53 (m, 3H), 3.17- 3.00 (m, 4H), 2.32- 2.29 (m, 4H), 2.28- 2.26 (m, 1H), 1.93- 1.90 (m, 1H) |
| 06-48 | 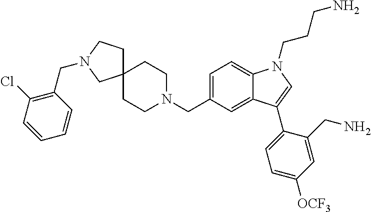 [SMILES: NCCCn1cc(-c2ccc(OC(F)(F)F)cc2CN)c2cc(CN3CCC4(CC3)CCN(Cc3ccccc3Cl)C4)ccc21] | 3-(3-(2- (aminomethyl)-4- (trifluoromethoxy) phenyl)-5-((2-(2- chlorobenzyl)-2,8- diazaspiro[4.5] decan-8-yl) methyl)-1H- indol-1-yl)propan- 1-amine diformate | Calc'd for C₃₅H₄₂Cl F₃N₅O: 640.3; Found: 640.4 | δ 8.42 (br s, 2H), 7.66 (d, J = 8.6 Hz, 1H), 7.62-7.59 (m, 2H), 7.58-7.55 (m, 2H), 7.54-7.51 (m, 1H), 7.45-7.37 (m, 3H), 7.33-7.28 (m, 2H), 4.46-4.41 (m, 2H), 4.32-4.27 (m, 2H), 4.21 (s, 2H), 3.96 (s, 2H), 3.18- 3.15 (m 1H), 3.12 (br s, 3H), 3.01-2.95 (m, 2H), 2.91 (br t, J = 6.9 Hz, 2H), 2.74 (s, 2H), 2.27 (quin, J = 7.3 Hz, 2H), 1.88- 1.79 (m, 6H) |
| 06-49 | 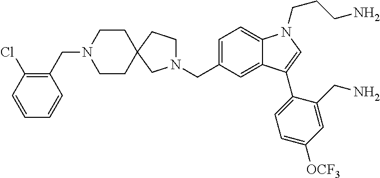 [SMILES: NCCCn1cc(-c2ccc(OC(F)(F)F)cc2CN)c2cc(CN3CCC4(CCN(Cc5ccccc5Cl)CC4)C3)ccc21] | 3-(3-(2- (aminomethyl)-4- (trifluoromethoxy) phenyl)-5-((8-(2- chlorobenzyl)-2,8- diazaspiro[4.5] decan-2-yl) methyl)-1H- indol-1-yl)propan- 1-amine dihydrochloride | Calc'd for C₃₅H₄₂Cl F₃N₅O: 640.3; Found: 640.4 | δ 7.72-7.68 (m, 3H), 7.60-7.56 (m, 4H), 7.49-7.44 (m, 4H), 4.52-4.45 (m, 5H), 4.27-4.27 (m, 2H), 3.58 (s, 1H), 3.49- 3.46 (m, 4H), 3.31- 3.29 (m, 2H), 3.07 (s, 1H), 2.28 (s, 3H), 2.14-1.98 (m, 7H). |
| 06-50 | 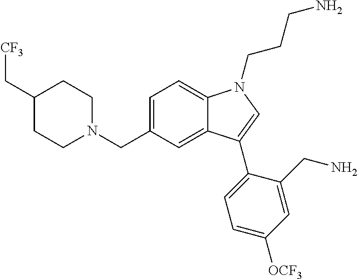 [SMILES: NCCCn1cc(-c2ccc(OC(F)(F)F)cc2CN)c2cc(CN3CCC(CC(F)(F)F)CC3)ccc21] | 3-(3-(2- (aminomethyl)-4- (trifluoromethoxy) phenyl)-5-((4- (2,2,2- trifluoroethyl) piperidin-1-yl) methyl)- 1H-indol-1- yl)propan-1-amine dihydrochloride | Calc'd for C₂₇H₁₂F₆ N₄O: 543.2; Found: 543.3 | δ 7.71 (d, J = 8.6 Hz, 1H), 7.68-7.62 (m, 3H), 7.61 (d, J = 2.0 Hz, 1H), 7.45 (dd, J = 1.5, 8.6 Hz, 2H), 4.46 (t, J = 7.1 Hz, 2H), 4.338 (s, 2H), 4.26 (s, 2H), 3.51- 3.42 (m, 2H), 3.08- 2.98 (m, 2H), 2.34- 2.15 (m, 2H, 2.01 (br s, 3H), 1.66 (br s, 2H) |
| 06-65 | 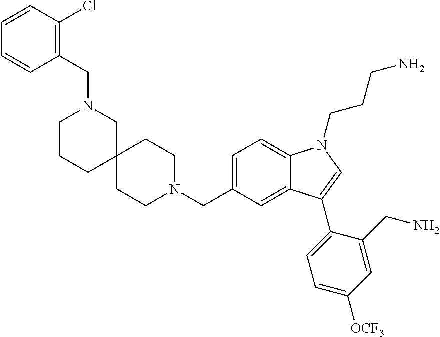 [SMILES: NCCCn1cc(-c2ccc(OC(F)(F)F)cc2CN)c2cc(CN3CCC4(CCCN(Cc5ccccc5Cl)C4)CC3)ccc21] | 3-(3-(2- (aminomethyl)-4- (trifluoromethoxy) phenyl)-5-((2-(2- chlorobenzyl)-2,9- diazaspiro[5.5] undecan-9-yl) methyl)- 1H-indol-1- yl)propan-1-amine dihydrochloride | Calc'd for C₃₆H₄₄Cl F₃N₅O: 654.3; Found: 640.4 | δ 8.00-7.79 (m, 1H), 7.74-7.67 (m, 2H), 7.66-7.59 (m, 3H), 7.56 (s, 1H), 7.54- 7.41 (m, 4H), 4.62- 4.35 (m, 6H), 4.27 (s, 1H), 4.32 (br s, 1H), 3.61-3.36 (m, 2H), 3.21 (br d, J = 10.8 Hz, 2H), 3.13-2.91 (m, 4H), 2.77-2.23 (m, 4H), 1.98-1.53 (m, 6H), 1.44-1.20 (m, 2H) |
| 06-84 | 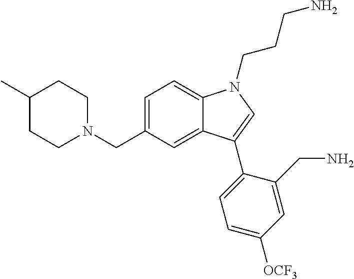 [SMILES: CC1CCN(Cc2ccc3c(c2)c(-c2ccc(OC(F)(F)F)cc2CN)cn3CCCN)CC1] | 3-(3-(2- (aminomethyl)-4- (trifluoromethoxy) phenyl)-5-((4- methylpiperidin-1- yl)methyl)-1H- indol-1-yl)propan- 1-amine dihydrochloride | Calc'd for C₂₆H₃₄F₃ N₄O: 475.3; Found: 475.3 | δ 7.72 (d, J = 8.4 Hz, 1H), 7.67-7.60 (m, 4H), 7.45 (br d, J = 8.4 Hz, 2H), 4.47 (br t, J = 7.1 Hz, 2H), 4.37 (s, 2H), 4.27 (s, 2H), 3.43 (br d, J = 11.9 Hz, 2H), 3.08- 2.91 (m, 4H), 2.30 (quin, J = 7.4 Hz, 2H), 1.87 (br d, J = 14.1 Hz, 2H), 1.69 (br s, 1H), 1.54-1.40 (m, 2H), 0.99 (d, J = 6.4 Hz, 3H) |
| 06-89 | 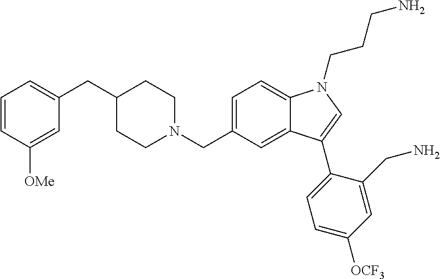 [SMILES: COc1cccc(CC2CCN(Cc3ccc4c(c3)c(-c3ccc(OC(F)(F)F)cc3CN)cn4CCCN)CC2)c1] | 3-(3-(2- (aminomethyl)-4- (trifluoromethoxy) phenyl)-5-((4-(3- methoxybenzyl) piperidin-1- yl)methyl)-1H- indol-1-yl)propan- 1-amine dihydrochloride | Calc'd for C₃₃H₄₀F₃ N₄O₂: 581.3; Found: 581.2 | □ 7.74-7.69 (m, 1H), 7.63 (br d, J = 2.4 Hz, 4H), 7.48-7.40 (m, 2H), 7.21-7.15 (m, 1H), 6.74 (br d, J = 6.8 Hz, 3H), 4.50- 4.43 (m, 2H), 4.50- 4.43 (m, 2H), 4.41- 4.30 (m, 2H), 3.77 (s, 3H), 3.49-3.39 (m, 2H), 3.07-2.89 (m, 4H), 2.62-2.53 (m, 2H), 2.34-2.23 (m, 2H), 1.93-1.82 (m, 3H), 1.62-1.41 (m, 2H) |
| 04-87 | 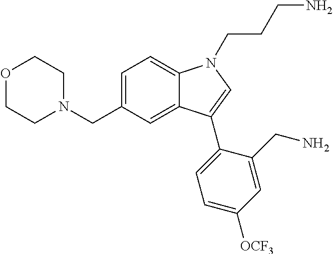 [SMILES: NCCCn1cc(-c2ccc(OC(F)(F)F)cc2CN)c2cc(CN3CCOCC3)ccc21] | 3-(3-2- (aminomethyl)-4- (trifluoromethoxy) phenyl)-5- (morpholinomethyl)- 1H-indol-1- yl)propan-1-amine dihydrochloride | Calc'd for C₂₄H₃₀F₃ N₄O₂: 463.2; Found: 463.3 | δ 7.75-7.68 (m, 2H), 7.67-7.60 (m, 3H), 7.52-7.42 (m, 2H), 4.52-4.41 (m, 4H), 4.27 (s, 2H), 4.01 (br d, J = 13.0 Hz, 2H), 3.82 (br t, J = 12.0 Hz, 2H), 3.35 (br d, J = 12.6 Hz, 2H), 3.25-3.14 (m, 2H), 3.06-2.97 (m, 2H), 2.35-2.24 (m, 2H) |
| 06-94 | 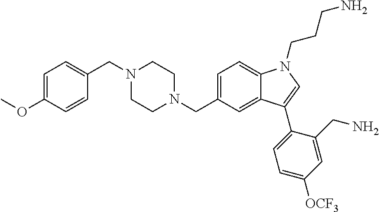 [SMILES: COc1ccc(CN2CCN(Cc3ccc4c(c3)c(-c3ccc(OC(F)(F)F)cc3CN)cn4CCCN)CC2)cc1] | 3-(3-(2- (aminomethyl)-4- (trifluoromethoxy) phenyl)-5-((4-(4- methoxybenzyl) piperazin-1- yl)methyl)-1H- indol-1-yl)propan- 1-amine dihydrochloride | Calc'd for C₃₂H₃₉F₃ N₅O₂: 582.3; Found: 582.4 | δ 7.75-7.70 (m, 2H), 7.69-7.60 (m, 3H), 7.52 (br d, J = 8.5 Hz, 3H), 7.46 (br d, J = 8.3 Hz, 1H), 7.03 (d, J = 8.5 Hz, 2H), 4.57 (s, 2H), 4.48 (br t, J = 7.0 Hz, 2H), 4.41 (s, 2H), 4.29 (s, 2H), 3.86- 3.81 (m, 3H), 3.65 (br s, 8H), 3.08-2.99 (m, 2H), 2.36-2.24 (m, 2H) |
| 06-92 | 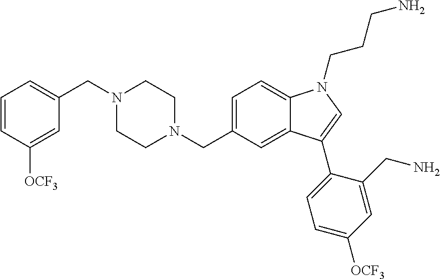 [SMILES: NCCCn1cc(-c2ccc(OC(F)(F)F)cc2CN)c2cc(CN3CCN(Cc4cccc(OC(F)(F)F)c4)CC3)ccc21] | 3-(3-(2- (aminomethyl)-4- (trifluoromethoxy) phenyl)-5-((4-(3- (trifluoromethoxy) benzyl)piperazin-1- yl)methyl)-1H- indol-1-yl)propan- 1-amine dihydrochloride | Calc'd for C₃₂H₃₆F₆ N₄O₂: 636.3; Found: 636.3 | δ 7.77-7.71 (m, 2H), 7.69-7.58 (m, 6H), 7.52 (br d, J = 9.0 Hz, 1H), 7.49-7.41 (m, 2H), 4.89 (s, 51H), 4.58 (s, 2H), 4.54-4.42 (m, 4H), 4.29 (s, 2H), 3.66 (br s, 8H), 3.33 (td, J = 1.5, 3.2 Hz, 8H), 3.07-2.96 (m, 2H), 2.37-2.24 (m, 2H) |
| 06-91 | 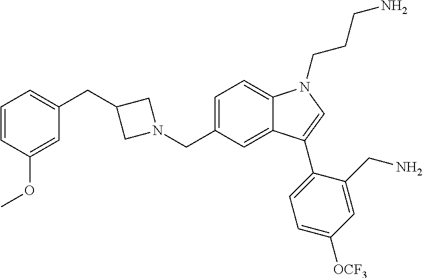 [SMILES: COc1cccc(CC2CN(Cc3ccc4c(c3)c(-c3ccc(OC(F)(F)F)cc3CN)cn4CCCN)C2)c1] | 3-(3-(2- (aminomethyl)-4- (trifluoromethoxy) phenyl)-5-((3-(3- methoxybenzyl) azetidin-1-yl) methyl)-1H-indol-1- yl)propan-1-amine diformate | Calc'd for C₃₁H₃₆F₃ N₅O₂: 553.3; Found: 553.3 | δ 8.49 (s, 2H), 7.64- 7.52 (m, 3H), 7.51- 7.45 (m, 2H), 7.41- 7.36 (m, 1H), 7.32 (d, J = 8.3 Hz, 1H), 7.18 (s, 1H), 6.72 (s, 3H), 4.41 (s, 2H), 4.20 (s, 2H), 4.07 (s, 2H), 3.93-3.85 (m, 2H), 3.75 (s, 3H), 3.67 (s, 2H), 3.10-3.00 (m, 1H), 2.98-2.91 (m, 2H), 2.88 (d, J = 7.5 Hz, 2H), 2.28-2.19 (m, 2H) |
| 04-92 | 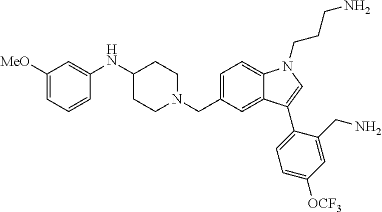 [SMILES: COc1cccc(NC2CCN(Cc3ccc4c(c3)c(-c3ccc(OC(F)(F)F)cc3CN)cn4CCCN)CC2)c1] | 1-((3-(2- (aminomethyl)-4- (trifluoromethoxy) phenyl)-1-(3- aminopropyl)-1H- indol-5-yl)methyl)- N-(3- methoxyphenyl) piperidin-4-amine dihydrochloride | Calc'd for C₃₂H₃₉F₃ N₅O₂: 582.3; Found: 582.4 | δ 7.81-7.70 (m, 2H), 7.70-7.60 (m, 4H), 7.60-7.34 (m, 4H), 7.13-7.05 (m, 1H), 4.89 (s, 36H), 4.52- 4.44 (m, 4H), 4.29 (s, 2H), 3.99 (td, J = 5.3, 10.7 Hz, 1H), 3.88 (s, 3H), 3.61 (br d, J = 12.9 Hz, 2H), 3.33 (s, 6H), 3.27-3.16 (m, 2H), 3.05-3.00 (m, 2H), 2.35-2.21 (m, 6H) |
| 04-93 | 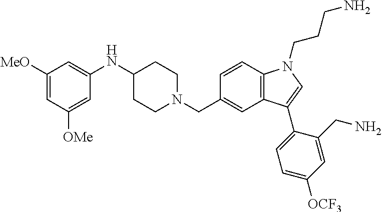 [SMILES: COc1cc(NC2CCN(Cc3ccc4c(c3)c(-c3ccc(OC(F)(F)F)cc3CN)cn4CCCN)CC2)cc(OC)c1] | 1-((3-(2- (aminomethyl)-4- (trifluoromethoxy) phenyl)-1-(3- aminopropyl)-1H- indol-5-yl)methyl)- N-(3,5- dimethoxyphenyl) piperidin-4-amine dihydrochloride | Calc'd for C₃₃H₄₁F₃ N₅O₃: 612.3; Found: 612.5 | δ 10.69 (br s, 1H), 8.65 (br s, 2H), 8.19 (br s, 2H), 7.79 (br d, J = 3.5 Hz, 2H), 7.75- 7.66 (m, 2H), 7.63 (d, J = 8.6 Hz, 1H), 7.52- 7.39 (m, 2H), 6.06- 5.67 (m, 3H), 4.44- 4.28 (m, 4H), 4.09 (br s, 2H), 3.62 (s, 6H), 3.60-3.37 (m, 37H), 3.13 (br s, 1H), 3.35-3.09 (m, 3H), 3.08-2.84 (m, 2H), 2.76 (br s, 2H), 2.47 (s, 57H), 2.18-1.98 (m, 4H), 1.75 (br d, J = 11.7 Hz, 1H) |
| 04-90 | 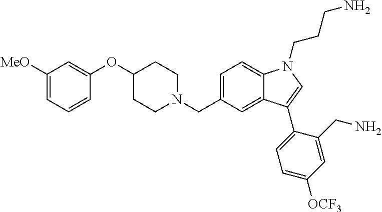 [SMILES: COc1cccc(OC2CCN(Cc3ccc4c(c3)c(-c3ccc(OC(F)(F)F)cc3CN)cn4CCCN)CC2)c1] | 3-(3-(2- (aminomethyl)-4- (trifluoromethoxy) phenyl)-5-((4-(3- methoxyphenoxy) piperidin-1- yl)methyl)-1H- indol-1-yl)propan- 1-amine dihydrochloride | Calc'd for C₃₂H₃₈F₃ N₄O₃: 583.3; Found: 583.3 | δ 7.75-7.59 (m, 5H), 7.51-7.42 (m, 2H), 7.22-7.13 (m, 1H), 6.61-6.48 (m, 3H), 4.51-4.41 (m, 4H), 4.27 (s, 2H), 3.79- 3.72 (m, 3H), 3.53 (br d, J = 12.7 Hz, 1H), 3.35 (br d, J = 3.8 Hz, 3H), 3.28- 3.11 (m, 1H), 3.06- 2.99 (m, 2H), 2.39- 2.24 (m, 3H), 2.11 (br s, 3H) |
| 04-91 | 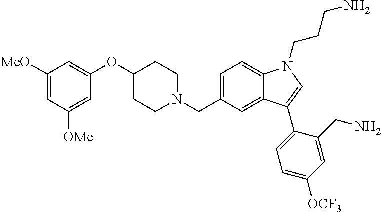 [SMILES: COc1cc(OC)cc(OC2CCN(Cc3ccc4c(c3)c(-c3ccc(OC(F)(F)F)cc3CN)cn4CCCN)CC2)c1] | 3-(3-(2- (aminomethyl)-4- (trifluoromethoxy) phenyl)-5-((4-(3,5- dimethoxyphenoxy) piperidin-1- yl)methyl)-1H- indol-1-yl)propan- 1-amine dihydrochloride | Calc'd for C₃₃H₄₀F₃ N₄O₄: 613.3; Found: 613.4 | δ 7.75-7.59 (m, 5H), 7.47 (br dd, J = 8.2, 16.8 Hz, 2H), 6.15- 6.10 (m, 3H), 4.51- 4.41 (m, 4H), 4.27 (s, 2H), 3.79-3.69 (m, 6H), 3.62-3.43 (m, 1H), 3.34 (br s, 3H), 3.20 (br t, J = 11.7 Hz, 1H), 3.06-2.98 (m, 2H), 2.39-2.23 (m, 3H), 2.16 (br s, 3H) |
| 04-84 | 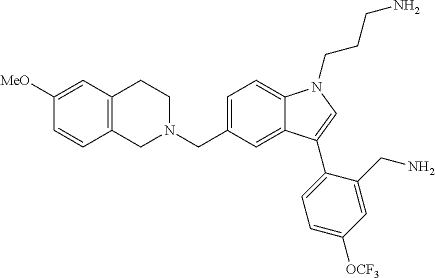 [SMILES: COc1ccc2c(c1)CCN(Cc1ccc3c(c1)c(-c1ccc(OC(F)(F)F)cc1CN)cn3CCCN)C2] | 3-(3-(2- (aminomethyl)-4- (trifluoromethoxy) phenyl)-5-((6- methoxy-3,4- dihydroisoquinolin- 2(1H)-yl)methyl)- 1H-indol-1- yl)propan-1-amine dihydrochloride | Calc'd for C₃₀H₃₄F₃ N₄O₂: 539.3; Found: 539.3 | δ 7.75 (d, J = 8.4 Hz, 1H), 7.69-7.63 (m, 3H), 7.60 (s, 1H), 7.51-7.42 (m, 2H), 7.04 (d, J = 8.2 Hz, 1H), 6.85-6.78 (m, 2H), 4.56 (br d, J = 5.1 Hz, 2H), 4.48 (t, J = 7.1 Hz, 2H), 4.33- 4.24 (m, 4H), 3.77 (s, 3H), 3.76-3.69 (m, 1H), 3.37 (br s, 1H), 3.24-3.08 (m, 2H), 3.07-2.99 (m, 2H), 2.37-2.26 (m, 2H) |
| 04-94 | 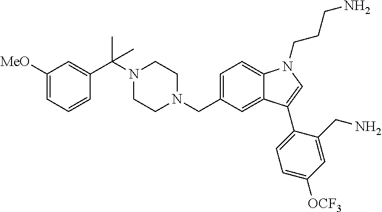 [SMILES: COc1cccc(C(C)(C)N2CCN(Cc3ccc4c(c3)c(-c3ccc(OC(F)(F)F)cc3CN)cn4CCCN)CC2)c1] | 3-(3-(2- (aminomethyl)-4- (trifluoromethoxy) phenyl)-5-((4-(2- (3-methoxyphenyl) propan-2-yl) piperazin- 1-yl)methyl)-1H- indol-1-yl)propan- 1-amine dihydrochloride | Calc'd for C₃₄H₄₃F₃ N₅O₂: 610.3: Found: 610.4 | δ 7.72-7.57 (m, 5H), 7.49-7.35 (m, 4H), 7.29 (br d, J = 7.7 Hz, 1H), 7.01 (br d J = 7.3 Hz, 1H), 4.53- 4.39 (m, 4H), 4.26 (s, 2H), 3.85 (s, 3H), 3.64 (br s, 4H), 3.53 (br s, 4H), 3.04-2.95 (m, 2H), 2.34-2.20 (m, 2H), 1.87 (br s, 6H) |
| 04-95 | 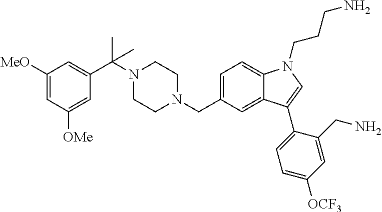 [SMILES: COc1cc(OC)cc(C(C)(C)N2CCN(Cc3ccc4c(c3)c(-c3ccc(OC(F)(F)F)cc3CN)cn4CCCN)CC2)c1] | 3-(3-(2- (aminomethyl)-4- (trifluoromethoxy) phenyl)-5-((4-(2- (3,5- dimethoxyphenyl) propan-2- yl)piperazin-1- yl)methyl)-1H- indol-1-yl)propan- 1-amine dihydrochloride | Calc'd for C₃₅H₄₅F₃ N₅O₃: 640.3; Found: 640.4 | δ 7.71-7.58 (m, 5H), 7.50-7.41 (m, 2H), 6.93 (s, 2H), 6.56 (s, 1H), 4.50 (s, 2H), 4.45 (br t, J = 7.0 Hz, 2H), 4.26 (s, 2H), 3.83 (s, 6H), 3.76 (br s, 2H), 3.70-3.46 (m, 6H), 3.00 (br t, J = 7.6 Hz, 2H), 2.28 (quin, J = 7.4 Hz, 2H), 1.85 (br s, 6H) |
| 04-97 | 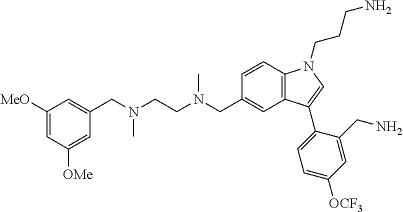 [SMILES: COc1cc(CN(C)CCN(C)Cc2ccc3c(c2)c(-c2ccc(OC(F)(F)F)cc2CN)cn3CCCN)cc(OC)c1] | N1-((3-(2- (aminomethyl)-4- (trifluoromethoxy) phenyl)-1-(3-amino propyl)-1H-indol- 5-yl)methyl)-N2- (3,5- dimethoxybenzyl)- N1,N2-dimethyl ethane-1,2-diamine dihydrochloride | Calc'd for C₃₃H₄₃F₃ N₅O₃: 614.3; Found: 614.5 | δ 7.66 (s, 5H), 7.55- 7.42 (m, 2H), 6.82 (d, J = 2.1 Hz, 2H), 6.59 (s, 1H), 4.49 (br t, J = 6.9 Hz, 4H), 4.31 (br d, J = 8.0 Hz, 4H), 3.97-3.58 (m, 10H), 3.08-2.98 (m, 2H), 2.82 (br s, 6H), 2.38- 2.24 (m, 2H) |
| 05-51 | 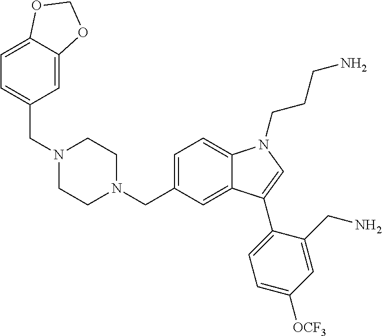 [SMILES: NCCCn1cc(-c2ccc(OC(F)(F)F)cc2CN)c2cc(CN3CCN(Cc4ccc5c(c4)OCO5)CC3)ccc21] | 3-[3-[2- (aminomethyl)-4- (trifluoromethoxy) phenyl]-5-[[4-(1,3- benzodioxol-5- ylmethyl)piperazin- 1-yl]methyl]indol- 1-yl]propan-1- amine dihydrochloride | Calc'd for C₃₂H₃₆F₃ N₅O₃: 596.3 Found: 596.5 | δ7.73-7.67 (m, 2H), 7.63 (d, J = 8.6 Hz, 1H), 7.61 (s, 1H), 7.58 (s, 1H), 7.49 (d, J = 8.6 Hz, 1H), 7.44 (br d, J = 8.4 Hz, 1H), 7.09 (s, 1H), 7.05 (br d, J = 8.2 Hz, 1H), 6.89 (d, J = 7.7 Hz, 1H), 6.00 (s, 2H), 4.87 (s, 2H), 4.55 (br s, 2H), 4.45- 4.43 (m, 2H), 4.27 (s, 2H), 3.81-3.43 (m, 8H), 3.32-3.28 (m, 11H), 3.05-2.94 (m, 2H), 2.27 (quin, J = 7.4 Hz, 2H) |
| 05-52 | 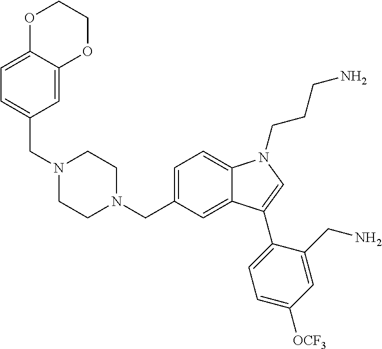 [SMILES: NCCCn1cc(-c2ccc(OC(F)(F)F)cc2CN)c2cc(CN3CCN(Cc4ccc5c(c4)OCCO5)CC3)ccc21] | 3-[3-[2- (aminomethyl)-4- (trifluoromethoxy) phenyl]-5-[[4-(2,3- dihydro-1,4- benzodioxin-6- ylmethyl)piperazin- 1-yl]methyl]indol- 1-yl]propan-1- amine dihydrochloride | Calc'd for C₃₃H₃₈F₃ N₅O₃: 610.3 Found: 610.5 | δ7.74-7.69 (m, 2H), 7.67-7.62 (m, 2H), 7.60 (s, 1H), 7.50 (br d, J = 8.8 Hz, 1H), 7.44 (br d, J = 8.2 Hz, 1H), 7.11 (d, J = 1.5 Hz, 1H), 7.04 (br d, J = 8.4 Hz, 1H), 6.91 (d, J = 8.4 Hz, 1H), 4.57 (br s, 2H), 4.46 (br t, J = 7.1 Hz, 2H), 4.36 (br s, 2H), 4.31-4.20 (m, 6H), 3.80-3.48 (m, 8H), 3.06-2.96 (m, 2H), 2.35-2.23 (m, 2H)). |
| 05-53 | 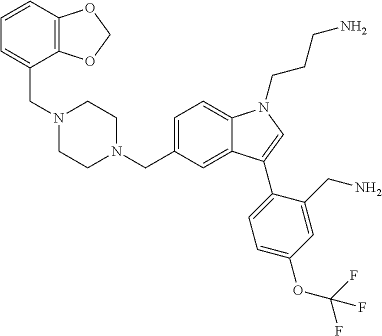 [SMILES: NCCCn1cc(-c2ccc(OC(F)(F)F)cc2CN)c2cc(CN3CCN(Cc4cccc5c4OCO5)CC3)ccc21] | 3-[3-[2- (aminomethyl)-4- (trifluoromethoxy) phenyl]-5-[[4-(1,3- benzodioxol-4- ylmethyl)piperazin- 1-yl]methyl]indol- 1-yl]propan-1- amine dihydrochloride | Calc'd for C₃₂H₃₆F₃ N₅O₃: 596.3 Found: 596.3 | δ7.70 (d, J = 7.7 Hz, 2H), 7.67-7.57 (m, 3H), 7.50 (d, J = 8.6 Hz, 1H), 7.43 (br d, J = 8.4 Hz, 1H), 7.05- 6.99 (m, 1H), 6.98- 6.89 (m, 2H), 6.05 (s, 2H), 4.56 (s, 2H), 4.49-4.38 (m, 4H), 4.27 (s, 2H), 3.91- 3.49 (m, 8H), 3.08- 2.91 (m, 2H), 2.28 (quin, J = 7.4 Hz, 2H) |
| 05-54 | 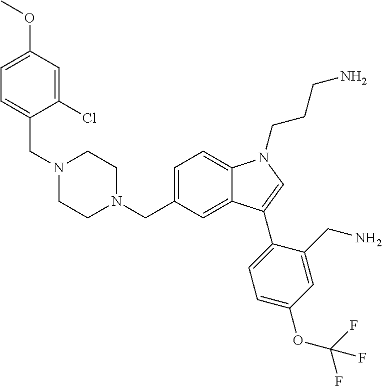 [SMILES: COc1ccc(CN2CCN(Cc3ccc4c(c3)c(-c3ccc(OC(F)(F)F)cc3CN)cn4CCCN)CC2)c(Cl)c1] | 3-[3-[2- (aminomethyl)-4- (trifluoromethoxy) phenyl]-5-[[4-[(2- chloro-4-methoxy- phenyl)methyl] piperazin-1- yl]methyl]indol-1- yl]propan-1-amine dihydrochloride | Calc'd for C₃₂H₃₇Cl F₃N₅O₂: 616.3 Found: 616.2 | δ7.75-7.69 (m, 2H), 7.67-7.58 (m, 4H), 7.50 (d, J = 9.7 Hz, 1H), 7.45 (br d, J = 8.4 Hz, 1H), 7.12 (d, J = 2.4 Hz, 1H), 7.01 (dd, J = 2.5, 8.7 Hz, 1H), 4.54 (s, 2H), 4.47 (br t, J = 6.9 Hz, 4H), 4.28 (s, 2H), 3.84 (s, 3H), 3.63 (br s, 8H), 3.07-2.97 (m, 2H), 2.29 (quin, J = 7.5 Hz, 2H) |
| 05-55 | 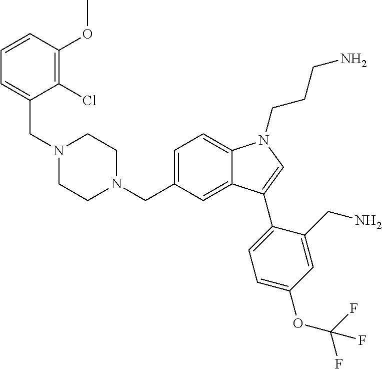 [SMILES: COc1cccc(CN2CCN(Cc3ccc4c(c3)c(-c3ccc(OC(F)(F)F)cc3CN)cn4CCCN)CC2)c1Cl] | 3-[3-[2- (aminomethyl)-4- (trifluoromethoxy) phenyl]-5-[[4-[(2- chloro-3-methoxy- phenyl)methyl] piperazin-1- yl]methyl]indol-1- yl]propan-1-amine dihydrochloride | Calc'd for C₃₂H₃₇Cl F₃N₅O₂: 616.3 Found: 616.3 | δ7.73-7.68 (m, 2H), 7.67-7.61 (m, 2H), 7.58 (s, 1H), 7.50 (d, J = 8.3 Hz, 1H), 7.45-7.38 (m, 2H), 7.36-7.33 (m, 1H), 7.24 (d, J = 8.3 Hz, 1H), 4.85 (s, 3H), 4.62 (s, 3H), 4.56- 4.43 (m, 2H), 4.27 (s, 2H), 3.91 (s, 3H), 3.78-3.60 (m, 8H), 3.04-2.96 (m, 2H), 2.31-2.24 (m, 2H) |
| 05-56 | 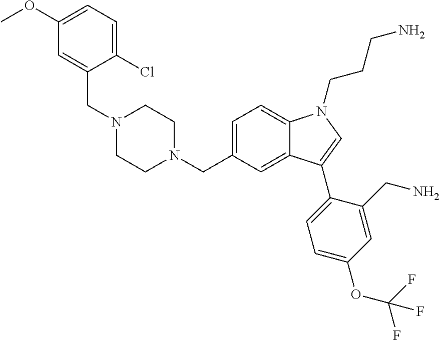 [SMILES: COc1ccc(Cl)c(CN2CCN(Cc3ccc4c(c3)c(-c3ccc(OC(F)(F)F)cc3CN)cn4CCCN)CC2)c1] | 3-[3-[2- (aminomethyl)-4- (trifluoromethoxy) phenyl]-5-[[4-[(2- chloro-5-methoxy- phenyl)methyl] piperazin-1- yl]methyl]indol-1- yl]propan-1-amine dihydrochloride | Calc'd for C₃₂H₃₇Cl F₃N₅O₂: 616.3 Found: 616.3 | δ7.70 (br d, J = 5.3 Hz, 2H), 7.66-7.61 (m, 2H), 7.58 (s, 1H), 7.50 (d, J = 8.3 Hz, 1H), 7.46- 7.39 (m, 3H), 7.04 (dd, J = 2.9, 9.0 Hz, 1H), 4.55 (br d, J = 4.4 Hz, 4H), 4.45 (t, J = 7.0 Hz, 2H), 4.27 (s, 2H), 3.85 (s, 3H), 3.72- 3.68 (m, 8H), 3.03- 2.96 (m, 2H), 2.33- 2.21 (m, 2H) |
| 05-58 | 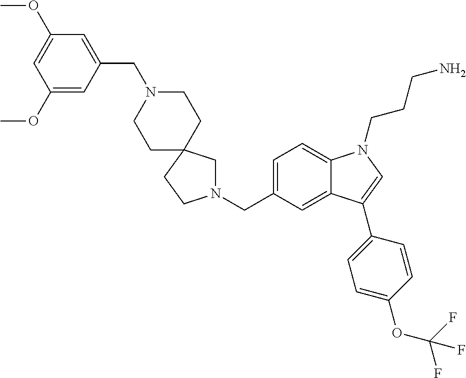 [SMILES: COc1cc(CN2CCC3(CC2)CCN(Cc2ccc4c(c2)c(-c2ccc(OC(F)(F)F)cc2)cn4CCCN)C3)cc(OC)c1] | 3-[5-[[8-[(3,5- dimethoxyphenyl) methyl]-2,8- diazaspiro[4.5] decan-2-yl]methyl)- 3-[4- (trifluoromethoxy) phenyl]indol-1- yl]propan-1-amine dihydrochloride | Calc'd for C₃₆H₄₃F₃ N₄O₃: 637.3 Found: 637.6 | δ8.16 (d, J = 17.2 Hz, 1H), 7.85-7.78 (m, 2H), 7.72-7.63 (m, 2H), 7.55-7.45 (m, 1H), 7.36 (dd, J = 3.1, 8.4 Hz, 2H), 6.71 (dd, J = 2.2, 9.0 Hz, 2H), 6.57 (t, J = 2.1 Hz, 1H), 4.59-4.47 (m, 2H), 4.42 (dt, J = 3.7, 6.8 Hz, 2H), 4.24 (s, 2H), 3.80 (s, 6H), 3.44-3.40 (m, 1H), 3.36-3.30 (m, 5H), 3.00-2.92 (m, 4H), 2.32-1.86 (m, 8H) |
| 05-59 | 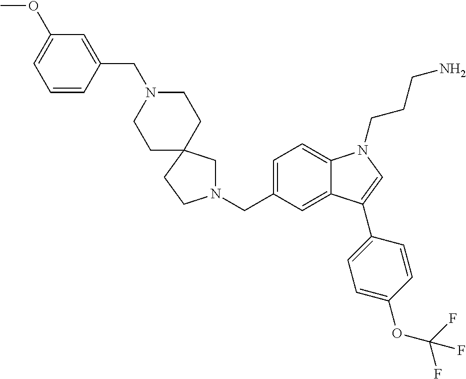 [SMILES: COc1cccc(CN2CCC3(CC2)CCN(Cc2ccc4c(c2)c(-c2ccc(OC(F)(F)F)cc2)cn4CCCN)C3)c1] | 3-[5-[[8-[(3- methoxyphenyl) methyl]-2,8- diazaspiro[4.5] decan-2-yl]methyl]- 3-[4- (trifluoromethoxy) phenyl]indol-1- yl]propan-1-amine dihydrochloride | Calc'd for C₃₅H₄₁F₃ N₄O₂: 607.3 Found: 607.6 | δ8.16 (br d, J = 17.9 Hz, 1H), 7.86-7.76 (m, 2H), 7.72-7.63 (m, 2H), 7.55-7.45 (m, 1H), 7.41-7.31 (m, 3H), 7.14 (br d, J = 9.7 Hz, 1H), 7.11- 7.00 (m, 2H), 4.62- 4.47 (m, 2H), 4.42- 4.39-4.37 (m, 2H), 4.29 (s, 2H), 3.82 (s, 3H), 3.74-3.31 (m, 6H), 3.19-2.94 (m, 4H), 2.26-1.92 (m, 8H) |

### Example 30: Synthesis of 4-[(3-methoxyphenyl)methyl]piperidine (06-89-4)

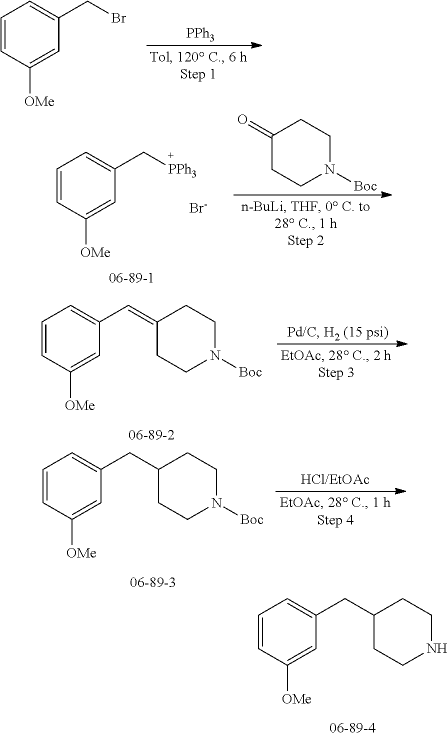

### Step 1: (3-methoxybenzyl)triphenylphosphonium (06-89-1)

A flask was fitted with 1-(bromomethyl)-3-methoxy-benzene (9.00 g, 44.8 mmol, 6.25 mL, 1.0 eq) and triphenylphosphine (13.4 g, 51.0 mmol, 1.14 eq) in toluene (120 mL). The reaction mixture was heated to 120° C. for 6 h under N₂. The suspension was filtered and the solid collected was washed with cold toluene (150 mL) and dried under vacuum to give compound 06-89-1 (20.0 g, 89% yield). M+H⁺=383.1 (LCMS). ¹H NMR (MeOD, 400 MHz): δ 7.91 (s, 3H), 7.79-7.63 (m, 12H), 7.20-7.12 (m, 1H), 6.89-6.83 (m, 1H), 6.62-6.57 (m, 1H), 6.50-6.46 (m, 1H), 5.18-5.07 (m, 2H), 3.49 (s, 3H).

### Step 2: tert-butyl 4-[(3-methoxyphenyl)methylene]piperidine-1-carboxylate (06-89-2)

To a mixture of 06-89-1 (6.98 g, 15.1 mmol, 2.0 eq) in THF (15 mL) at 0° C. was n-BuLi (2.5 M, 6.62 mL, 2.2 eq) slowly. The resulting mixture was stirred at 0° C. for 30 min before tert-butyl 4-oxopiperidine-1-carboxylate (1.50 g, 7.53 mmol, 1.0 eq) in THF (10 mL) was added. The reaction mixture was stirred at 28° C. for 1.5 h, poured into H₂O (30 mL) and extracted with EtOAc (35 mL*3). The combined organic layers were washed with brine (35 mL), dried over anhydrous Na₂SO₄, filtered and concentrated. The residue was purified by column chromatography (SiO₂) to give compound 06-89-2 (1.60 g, 64% yield). M+H⁺=248.1 (LCMS).

### Step 3:tert-butyl 4-[(3-methoxyphenyl)methyl]piperidine-1-carboxylate (06-89-3)

To a solution of compound 06-89-2 (800.0 mg, 2.64 mmol, 1.0 eq) in EtOAc (15 mL) was added Pd/C (500.0 mg) under N₂. The suspension was degassed under vacuum and purged with H₂ several times. The mixture was stirred under H₂ (15 psi) at 28° C. for 2 h. The reaction mixture was filtered through celite and the filtrate was evaporated to give compound 06-89-3 (1.38 g, 86% yield). ¹H NMR (CDCl₃, 400 MHz): δ 7.20 (t, J=7.89 Hz, 1H), 6.77-6.72 (m, 2H), 6.71-6.68 (m, 1H), 4.08-4.01 (m, 1H), 3.81 (s, 3H), 2.70-2.59 (m, 2H), 2.52 (d, J=6.58 Hz, 2H), 1.75-1.57 (m, 4H), 1.46 (s, 9H), 1.22-1.08 (m, 2H).

### Step 4: 4-[(3-methoxyphenyl)methyl]piperidine (06-89-4)

A flask was fitted with 06-89-3 (1.38 g, 4.52 mmol, 1.0 eq) in HCl/EtOAc (15 mL) and EtOAc (4 mL). The reaction mixture was stirred at 28° C. for 1 h. The reaction mixture was evaporated to give the residue. The residue was dissolved in water (10 mL), adjusted with saturated NaHCO₃ solution and extracted with EtOAc (35 mL*2). The combined organic layers were dried over anhydrous Na₂SO₄, filtered and evaporated to give compound 06-89-4 (800.0 mg, 78% yield). M+H⁺=206.2 (LCMS).

### Example 31: Synthesis of 4-(3,5-dimethoxyphenoxy)piperidine (04-91-2)

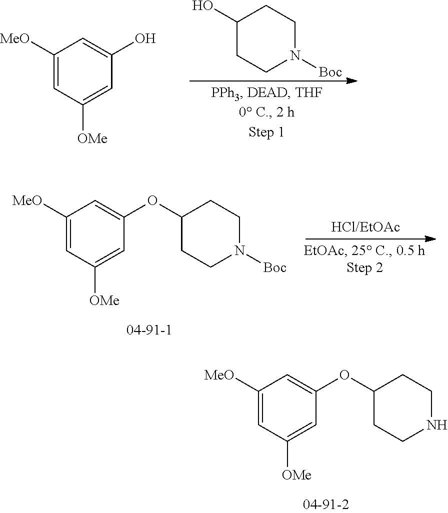 [SMILES: COc1cc(O)cc(OC)c1 ; CC(C)(C)OC(=O)N1CCC(O)CC1 ; COc1cc(OC)cc(OC2CCN(C(=O)OC(C)(C)C)CC2)c1 ; "HCl/EtOAc" ; COc1cc(OC)cc(OC2CCNCC2)c1]

### Step 1: tert-butyl 4-(3,5-dimethoxyphenoxy)piperidine-1-carboxylate (04-91-1)

To a solution of 3,5-dimethoxyphenol (5.00 g, 32.4 mmol, 1.0 eq) and tert-butyl 4-hydroxypiperidine-1-carboxylate (6.53 g, 32.4 mmol, 1.0 eq) in THF (80 mL) was added PPh₃ (10.2 g, 38.9 mmol, 1.2 eq) and DEAD (6.78 g, 38.9 mmol, 7.1 mL, 1.2 eq) at 0° C. The mixture was stirred at 0° C. for 2 h under N₂, poured into H₂O (300 mL) and extracted with EtOAc (10 mL*3). The combined organic layers were washed with brine (150 mL), dried over anhydrous Na₂SO₄, filtered and the filtrate was concentrated in vacuum. The residue was purified by column chromatography (SiO₂) to give compound 04-91-1 (5.00 g, 12.9 mmol, 40% yield). M+H⁺=338.2 (LCMS). ¹H NMR (CDCl₃, 400 MHz): δ 6.08 (s, 3H), 4.40 (tt, J=3.5, 7.0 Hz, 1H), 3.75 (s, 6H), 3.48 (t, J=5.7 Hz, 2H), 3.35-3.26 (m, 2H), 1.95-1.85 (m, 2H), 1.78-1.68 (m, 2H), 1.46 (s, 9H).

### Step 2: 4-(3,5-dimethoxyphenoxy)piperidine (04-91-2)

To a solution of 04-91-1 (300.0 mg, 889.1 μmol, 1.0 eq) in EtOAc (2 mL) was added HCl/EtOAc (4 M, 2.00 mL, 9.0 eq). The mixture was stirred at 25° C. for 0.5 h. The reaction mixture was poured into saturated sodium carbonate solution (200 mL). The mixture was extracted with DCM (50 mL*8). The combined organic layers were washed with brine (200 mL), dried over anhydrous Na₂SO₄, filtered and the filtrate was concentrated in vacuum to give compound 04-91-2 (100.0 mg, crude). M+H⁺=238.2 (LCMS).

### Example 32: Synthesis of 1-[1-(3-methoxyphenyl)-1-methyl-ethyl]piperazine (04-94-3)

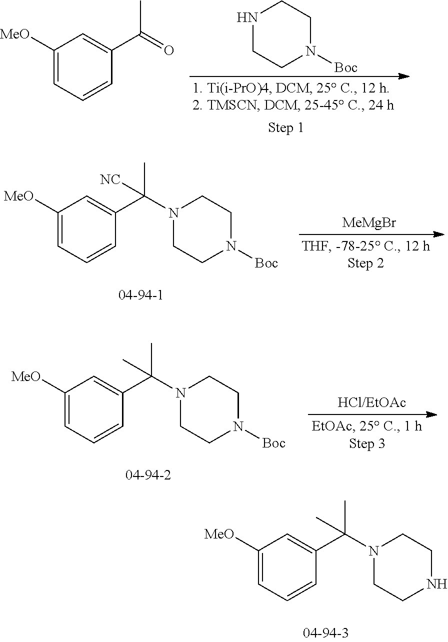

### Step 1: tert-butyl 4-[1-cyano-1-(3-methoxyphenyl)ethyl]piperazine-1-carboxylate (04-94-1)

To a solution of 1-(3-methoxyphenyl)ethanone (5.00 g, 33.3 mmol, 4.59 mL, 1.0 eq) in DCM (100 mL) was added tert-butyl piperazine-1-carboxylate (6.20 g, 33.3 mmol, 1.0 eq) and Ti(i-PrO)₄ (9.46 g, 33.3 mmol, 9.85 mL, 1.0 eq) stirred at 25° C. The resulting mixture was stirred at 25° C. for 12 h and TMSCN (4.95 g, 49.9 mmol, 6.27 mL, 1.5 eq) was added dropwise. After another 12 h, the reaction mixture was heated at 45° C. for 12 h, cooled to rt and diluted with water (100 mL). The organic layer was separated and the aqueous phase was extracted with DCM (40 mL*3). The combined organic layers were washed with brine (100 mL), dried over anhydrous Na₂SO₄, then filtered and the filtrate was concentrated under reduced pressure. The residue was purified by column chromatography (SiO₂) to give compound 04-94-1 (8.00 g, 14.96 mmol, 45% yield). ¹H NMR (CDCl₃, 400 MHz): δ 7.32-7.25 (m, 1H), 7.17 (d, J=7.9 Hz, 1H), 7.12 (t, J=2.0 Hz, 1H), 6.86 (dd, J=1.8, 7.9 Hz, 1H), 3.81 (s, 3H), 3.43 (br s, 4H), 2.60 (br d, J=11.0 Hz, 2H), 2.46-2.37 (m, 2H), 1.71 (s, 3H), 1.44 (s, 9H).

### Step 2: tert-butyl 4-[1-(3-methoxyphenyl)-1-methyl-ethyl]piperazine-1-carboxylate (04-94-2)

To a stirred solution of 04-94-1 (1.00 g, 2.89 mmol, 1.0 eq) in THF (20 mL) was added MeMgBr (3 M, 5.78 mL, 6.0 eq) dropwise at −78° C. The resulting mixture was stirred at 25° C. for 12 h, then added to saturated NH₄Cl (50 mL) solution slowly. The mixture was extracted with EtOAc (30 mL*3). The organic layer was washed with saturated NaHCO₃ solution (50 mL), brine (50 mL), dried over anhydrous Na₂SO₄, then filtered and the filtrate was concentrated under reduced pressure. The residue was purified by column chromatography (SiO₂) to give compound 04-94-2 (800.0 mg, 2.39 mmol, 82% yield). ¹H NMR (CDCl₃, 400 MHz): δ 7.20-7.11 (m, 1H), 7.07 (s, 1H), 7.02 (d, J=7.9 Hz, 1H), 6.68 (dd, J=1.8, 8.2 Hz, 1H), 3.73 (s, 3H), 3.30 (br s, 4H), 2.35 (br s, 4H), 1.37 (s, 9H), 1.25 (s, 6H).

### Step 3: 1-[1-(3-methoxyphenyl)-1-methyl-ethyl]piperazine (04-94-3)

To a stirred solution of 04-94-2 (800.0 mg, 2.39 mmol, 1.0 eq) in EtOAc (1 mL) was added HCl/EtOAc (4 M, 5.00 mL, 8.4 eq). The reaction mixture was stirred at 25° C. for 1 h, diluted with water (20 mL), and extracted with EtOAc (10 mL*3). The aqueous phase was basified by NaOH powder to pH=9, and extracted with DCM (20 mL*4). The combined DCM layers were washed with brine (40 mL), dried over anhydrous Na₂SO₄, then filtered and the filtrated was concentrated under reduced pressure to give compound 04-94-3 (400.0 mg, 1.71 mmol, 71% yield). ¹H NMR (CDCl₃, 400 MHz): δ 7.25-7.17 (m, 1H), 7.13 (s, 1H), 7.07 (d, J=7.7 Hz, 1H), 6.72 (dd, J=1.7, 8.0 Hz, 1H), 3.79 (s, 3H), 2.83 (t, J=4.7 Hz, 4H), 2.43 (br s, 4H), 1.93 (br s, 1H), 1.29 (s, 6H).

### Example 33: Synthesis of 3-[3-[2-(aminomethyl)-4-methyl-phenyl]-5-[[1-[(2-chlorophenyl)methyl]-4-piperidyl]methyl]indol-1-yl]propan-1-amine (06-88)

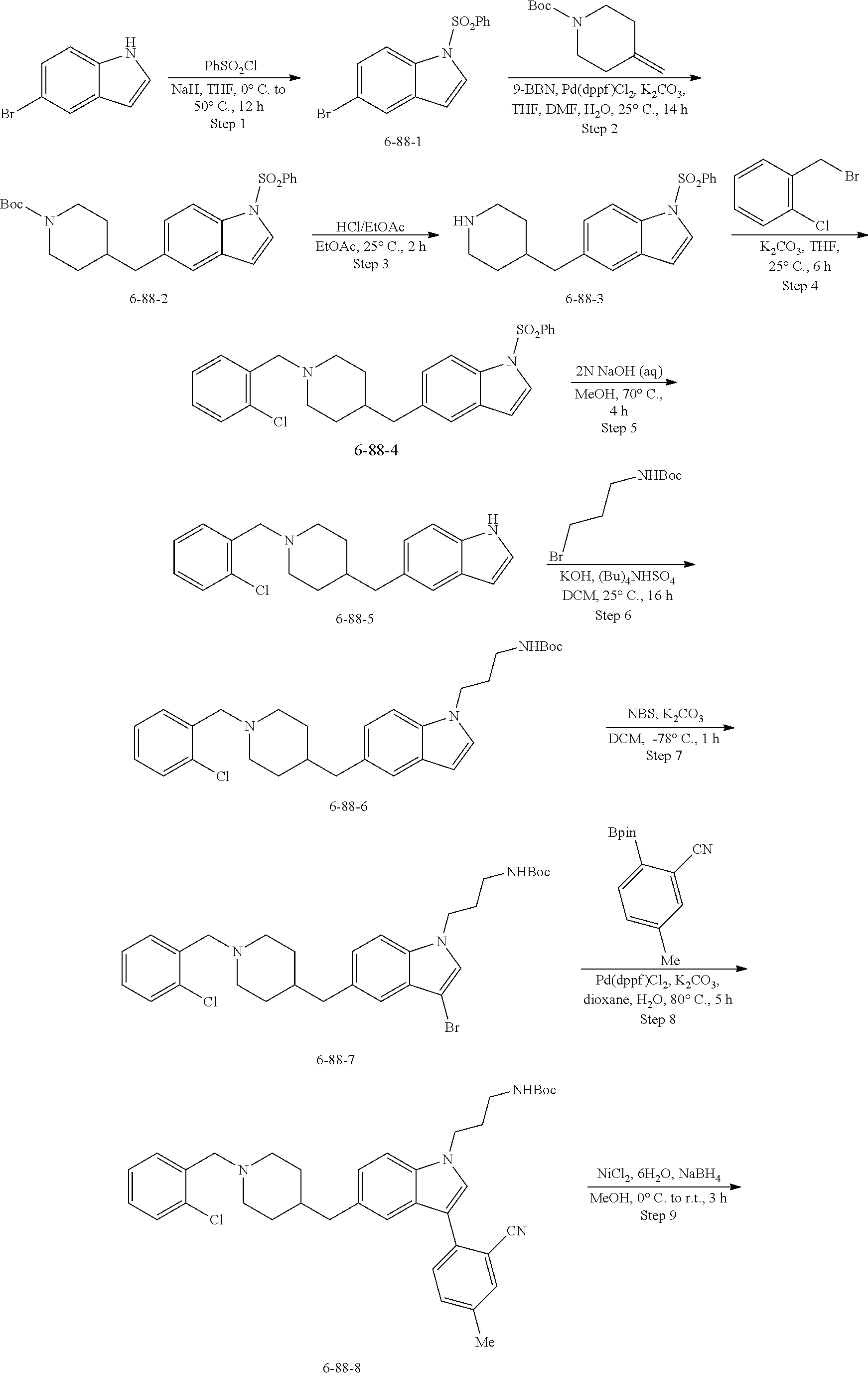

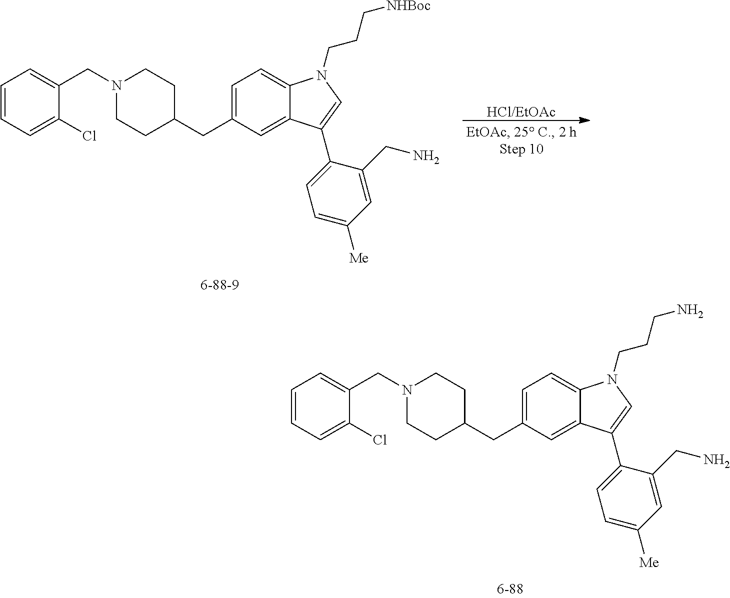 [SMILES: Cc1ccc(-c2cn(CCCNC(=O)OC(C)(C)C)c3ccc(CC4CCN(Cc5ccccc5Cl)CC4)cc23)c(CN)c1 ; "HCl/EtOAc" ; Cc1ccc(-c2cn(CCCN)c3ccc(CC4CCN(Cc5ccccc5Cl)CC4)cc23)c(CN)c1]

### Step 1: 1-(benzenesulfonyl)-5-bromoindole (06-88-1)

To a solution of NaH (2.45 g, 61.2 mmol, 1.2 eq) in THF (150 mL) was added 5-bromo-1H-indole (10.0 g, 51.0 mmol, 1.0 eq) in THF (50 mL) of at 0° C. After 30 min, a solution of benzenesulfonyl chloride (9.91 g, 56.1 mmol, 7.18 mL, 1.1 eq) in THF (50 mL) was added dropwise at 0° C. The mixture was stirred at 50° C. for 15.5 h, cooled to 25° C. and quenched by addition of saturated aq NH₄Cl (100 mL). The mixture was concentrated to remove organic solvent and extracted with EtOAc (100 mL*2). The combined organic layers were washed with H₂O (100 mL), dried over anhydrous Na₂SO₄ and filtered. The filtrate was concentrated under reduced pressure to give a residue which was purified by column chromatography (SiO₂) to give compound 06-88-1 (14.20 g, 26.77 mmol, 52% yield).

### Step 2:tert-butyl 4-[[1-(benzenesulfonyl)indol-5-yl]methyl]piperidine-1-carboxylate (06-88-2)

To a solution of tert-butyl 4-methylenepiperidine-1-carboxylate (2.83 g, 14.3 mmol, 2.47 mL, 1.0 eq) in THF (16.0 mL) was added 9-BBN (0.5 M, 28.6 mL, 1.0 eq) under N₂. The solution was heated to 70° C. for 1 h. After cooling to 20° C., the mixture was added to a mixture of 06-88-1 (7.60 g, 14.3 mmol, 1.0 eq), Pd(dppf)Cl₂.CH₂Cl₂ (585.0 mg, 716.4 μmol, 0.05 eq), DMF (40 mL), H₂O (40 mL) and K₂CO₃ (1.98 g, 14.3 mmol, 1.0 eq). The resulting mixture was heated to 70° C. for 7 h, cooled to 20° C. and poured into H₂O (100 mL). The mixture was extracted with EtOAc (100 mL*2). The combined organic layesr were washed with H₂O (100 mL*4), brine (100 mL), dried over anhydrous Na₂SO₄, and concentrated to give a residue. The residue was purified by column chromatography (SiO₂) to give compound 06-88-2 (4.90 g, 10.8 mmol, 75% yield).

### Step 3: 1-(benzenesulfonyl)-5-(4-piperidylmethyl)indole (06-88-3)

To a solution of 06-88-2 (6.70 g, 14.7 mmol, 1.0 eq) in EtOAc (10 mL) was added HCl/EtOAc (4 M, 20 mL, 5.4 eq) dropwise at 0° C. After 2 h, the reaction mixture was concentrated. The residue was diluted with H₂O (10 mL), adjusted to pH=1 by addition of aqueous NaOH (1N), and extracted with DCM (30*2) mL. The combined organic layers were washed with H₂O (30 mL), dried over anhydrous Na₂SO₄, filtered and concentrated under reduced pressure to give compound 06-88-3 (4.00 g, 9.39 mmol, 64% yield).

### Step 4: 1-(benzenesulfonyl)-5-[[1-[(2-chlorophenyl)methyl]-4-piperidyl]methyl]indole (06-88-4)

To a solution of 06-88-3 (1.20 g, 3.39 mmol, 1.0 eq) in CH₃CN (20 mL) were added K₂CO₃ (1.17 g, 8.46 mmol, 2.50 eq) and 1-(bromomethyl)-2-chloro-benzene (695.6 mg, 3.39 mmol, 440.3 μL, 1.0 eq). After 16 h, H₂O (20 mL) was added and the mixture was concentrated to remove CH₃CN. The resulting mixture was extracted with DCM (50 mL). The organic layer was separated, washed with H₂O (30 mL), dried over anhydrous Na₂SO₄, filtered and concentrated to give a residue which was purified by column chromatography (SiO₂) to give compound 06-88-4 (1.20 g, 72% yield).

### Step 5: 5-[[1-[(2-chlorophenyl)methyl]-4-piperidyl]methyl]-1H-indole (06-88-5)

To a solution of 06-88-4 (1.20 g, 2.51 mmol, 1.00 eq) in MeOH (15.00 mL) was added aq NaOH (2 N, 10.00 mL, 7.97 eq). The mixture was heated to 70° C. and stirred for 6 h. The reaction mixture was diluted with DCM (40 mL) and stirred for 0.5 h, then filtered and the filtrate was concentrated under reduced pressure to give a residue. The residue was purified by column chromatography (SiO₂) to give compound 06-88-5 (560.0 mg, 1.65 mmol, 66% yield).

### Step 6: tert-butyl N-[3-[5-[[1-[(2-chlorophenyl)methyl]-4-piperidyl]methyl]indol-1-yl]propyl]carbamate (06-88-6)

To a solution of 06-88-5 (560.0 mg, 1.65 mmol, 1.0 eq) in DCM (10 mL) was added tert-butyl N-(3-bromopropyl)carbamate (392.90 mg, 1.65 mmol, 1.0 eq), tetrabutylammonium hydrogen sulfate (560.22 mg, 1.65 mmol, 1.0 eq) and KOH (231.45 mg, 4.13 mmol, 2.50 eq). The mixture was stirred at 25° C. for 16 h, quenched by addition of H₂O (25 mL), and extracted with DCM (30 mL*2). The combined organic layers were washed with H₂O (40 mL), brine (40 mL), dried over anhydrous Na₂SO₄ and filtered. The filtrate was concentrated under reduced pressure to give a residue which was purified by column chromatography (SiO₂) to give compound 06-88-6 (560.0 mg, 1.13 mmol, 68% yield).

### Step 7: tert-butyl N-[3-[3-bromo-5-[[1-[(2-chlorophenyl)methyl]-4-piperidyl]methyl]indol-1-yl]propyl]carbamate (06-88-7)

To a solution of 06-88-6 (770.0 mg, 1.55 mmol, 1.0 eq) in DCM (5 mL) was added NBS (276.3 mg, 1.55 mmol, 1.0 eq) and K₂CO₃ (536.3 mg, 3.88 mmol, 2.5 eq). After 3 h at −78° C., the reaction was quenched with saturated aq Na₂SO₃ (25 mL), and extracted with DCM (20 mL*2). The combined organic layers were washed with H₂O (20 mL), brine (20 mL), dried over anhydrous Na₂SO₄ and filtered. The filtrate was concentrated under reduced pressure to give a residue. The residue was purified by column chromatography (SiO₂) to give compound 06-88-7 (360.0 mg, 626.1 μmol, 40% yield).

### Step 8: tert-butyl N-[3-[5-[[1-[(2-chlorophenyl)methyl]-4-piperidyl]methyl]-3-(2-cyano-4-methyl-phenyl) indol-1-yl]propyl]carbamate (06-88-8)

A mixture of 06-88-7 (240.0 mg, 417.4 μmol, 1.0 eq), 5-methyl-2-(4,4,5,5-tetramethyl-1,3,2-dioxaborolan-2-yl)benzonitrile (101.5 mg, 417.4 μmol, 1.0 eq), Pd(dppf)Cl₂ (30.5 mg, 41.7 μmol, 0.1 eq) and K₂CO₃ (144.2 mg, 1.04 mmol, 2.5 eq) in dioxane (6.0 mL) and H₂O (1.5 mL) was degassed and purged with N₂ for 3 times, and then the mixture was stirred at 80° C. for 16 h under N₂. The mixture was cooled to rt, quenched with H₂O (15 mL) and extracted with DCM (20 mL*2). The combined organic layers were washed with H₂O (20 mL), brine (20 mL), dried over anhydrous Na₂SO₄ and filtered. The filtrate was concentrated under reduced pressure to give a residue. The residue was purified twice by prep-TLC (SiO₂) to give compound 06-88-8 (75.0 mg, 61.7 μmol, 15% yield).

### Step 9: tert-butyl N-[3-[3-[2-(aminomethyl)-4-methyl-phenyl]-5-[[1-[(2-chlorophenyl)methyl]-4-piperidyl]methyl]indol-1-yl]propyl]carbamate (06-88-9)

To a solution of 06-88-8 (75.0 mg, 61.7 μmol, 1.0 eq) in MeOH (2 mL) was added NiCl₂.6H₂O (14.7 mg, 61.7 μmol, 1.0 eq) and NaBH₄ (23.4 mg, 617.2 μmol, 10.0 eq) at 0° C. After addition, the black mixture was stirred 25° C. for 2 h and concentrated under reduced pressure. The residue was diluted with DCM (20 mL), washed with H₂O (10 mL), dried over anhydrous Na₂SO₄, filtered, and concentrated under reduced pressure to give a residue. The residue was purified by prep-TLC (SiO₂) to give compound 06-88-9 (31.0 mg, 45% yield).

### Step 10: 3-[3-[2-(aminomethyl)-4-methyl-phenyl]-5-[[1-[(2-chlorophenyl)methyl]-4-piperidyl]methyl]indol-1-yl]propan-1-amine (06-88)

To a solution of 06-88-9 (30.0 mg, 1.0 eq) in EtOAc (1 mL) was added HCl/EtOAc (4 M, 1.20 mL, 98.5 eq) dropwise slowly. The mixture was stirred at 25° C. for 2 h. The mixture was concentrated to give a residue, which was washed with EtOAc (1 mL*2) and dried under reduce pressure to give a crude compound 06-88 (25.0 mg, crude, HCl salt). The residue was purified by prep-HPLC (FA condition) to give compound 06-88 (3 mg, FA salt). M+H⁺=515.4 (LCMS). ¹H NMR (MeOD, 400 MHz): δ 8.78-8.30 (m, 2H), 7.52-7.37 (m, 4H), 7.36-7.25 (m, 5H), 7.12-7.07 (m, 2H), 4.36 (s, 2H), 4.11 (s, 2H), 3.79 (s, 2H), 3.07-2.99 (m, 2H), 2.97-2.89 (m, 2H), 2.61 (br s, 2H), 2.45 (s, 3H), 2.27 (br s, 4H), 1.64 (br s, 3H), 1.42-1.26 (m, 2H).

### Example 34: Synthesis of 3-[3-[2-(aminomethyl)-4-(trifluoromethoxy)phenyl]-5-[[1-[(3-methoxyphenyl)methyl]-4-piperidyl]methyl]indol-1-yl]propan-1-amine (06-90)

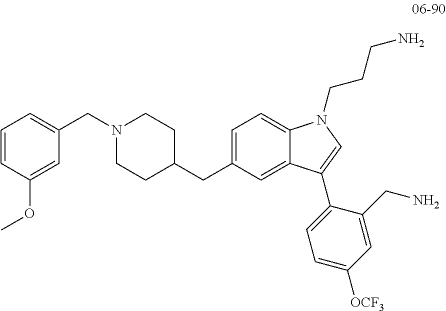 [SMILES: COc1cccc(CN2CCC(Cc3ccc4c(c3)c(-c3ccc(OC(F)(F)F)cc3CN)cn4CCCN)CC2)c1]

Compound 06-90 was prepared according to similar procedures as described for the synthesis of 06-88. M+H⁺=581.4 (LCMS). ¹H NMR (MeOD, 400 MHz): δ 7.60 (br d, J=8.3 Hz, 2H), 7.53 (d, J=8.4 Hz, 1H), 7.49-7.43 (m, 2H), 7.39 (t, J=8.0 Hz, 1H), 7.21-7.13 (m, 2H), 7.11 (s, 1H), 7.09-7.03 (m, 2H), 4.41 (br t, J=6.8 Hz, 2H), 4.24 (br d, J=3.5 Hz, 4H), 3.84 (s, 3H), 3.45 (br d, J=11.9 Hz, 2H), 3.04-2.91 (m, 4H), 2.70 (br d, J=6.5 Hz, 2H), 2.32-2.25 (m, 2H), 1.88 (br d, J=14.3 Hz, 3H), 1.53 (br d, J=14.1 Hz, 2H).

### Example 35: Synthesis of N-[[2-[1-(3-aminopropyl)-5-[[4-[(2-chlorophenyl)methyl]piperazin-1-yl]methyl]indol-3-yl]-5-methoxy-phenyl]methyl]methanesulfonamide (06-103)

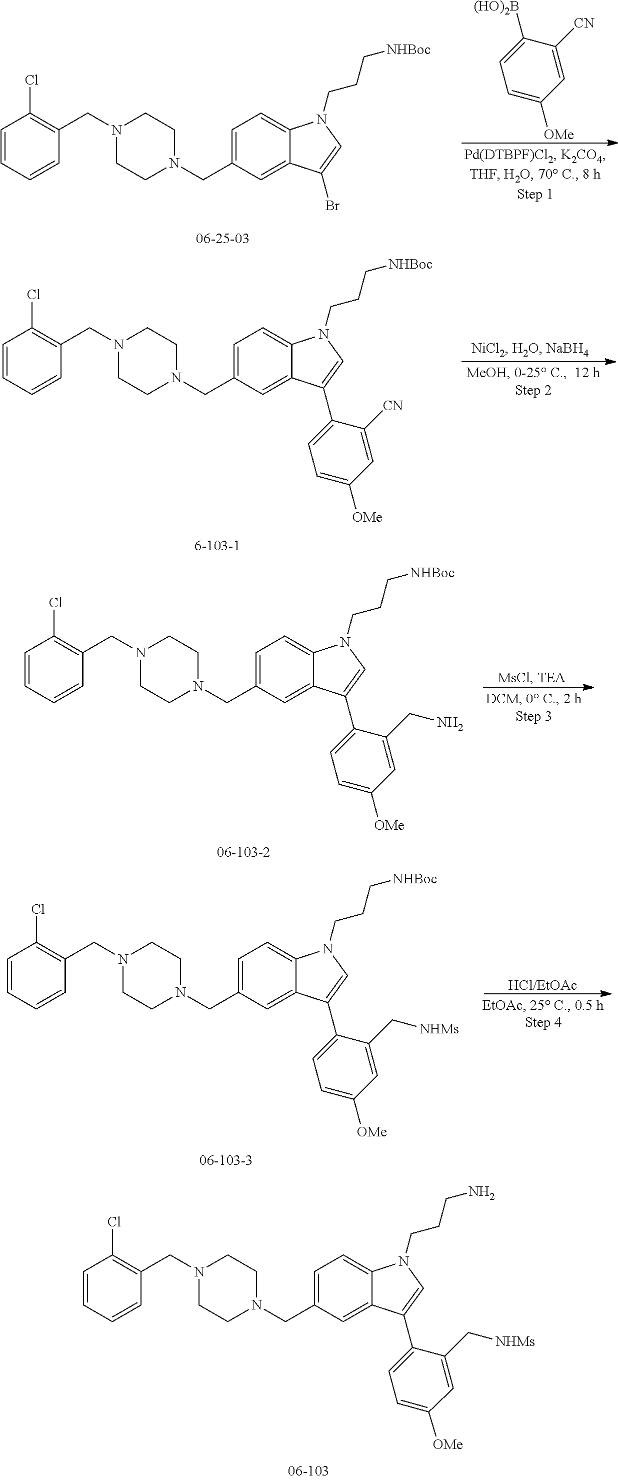

Step 1 and step 2 are carried out according to procedures as described in step 2 of the synthesis of 06-20 and step 1 of the synthesis of 06-21.

### tert-butyl N-[3-[5-[[4-[(2-chlorophenyl)methyl]piperazin-1-yl]methyl]-3-(2-cyano-4-methoxy-phenyl)indol-1-yl]propyl]carbamate (06-103-1)

M+H⁺=628.3 (LCMS).

### tert-butyl N-[3-[3-[2-(aminomethyl)-4-methoxy-phenyl]-5-[[4-[(2-chlorophenyl)methyl]piperazin-1-yl]methyl]indol-1-yl]propyl]carbamate (06-103-2)

M+H⁺=632.3 (LCMS).

### Step 3: tert-butyl N-[3-[5-[[4-[(2-chlorophenyl)methyl]piperazin-1-yl]methyl]-3-[2-(methanesulfonamidomethyl)-4-methoxy-phenyl]indol-1-yl]propyl]carbamate (06-103-3)

To a solution of 06-103-2 (180.0 mg, 284.71 μmol, 1.0 eq) in DCM (5 mL) was added TEA (80.0 mg, 790.6 μmol, 109.6 μL, 2.8 eq) and MsCl (90.0 mg, 785.8 μmol, 60.8 μL, 2.8 eq) at 0° C. The mixture was stirred at 0° C. for 2 h under N₂, poured into H₂O (150 mL) and extracted with DCM (50 mL*3). The combined organic layers were washed with brine (80 mL), dried over anhydrous Na₂SO₄, filtered and concentrated in vacuum to give a residue (200 mg), which was purified by prep-TLC (SiO₂) to give compound 06-103-3 (40 mg, 56.3 μmol, 20% yield). M+H⁺=710.2 (LCMS).

### Step 4: N-[[2-[1-(3-aminopropyl)-5-[[4-[(2-chlorophenyl)methyl]piperazin-1-yl]methyl]indol-3-yl]-5-methoxy-phenyl]methyl]methanesulfonamide (06-103)

To a solution of 06-103-3 (40.0 mg, 56.3 μmol, 1.0 eq) in EtOAc (1 mL) was added HCl/EtOAc (4 M, 1.00 mL, 71.0 eq). The mixture was stirred at 25° C. for 0.5 h. The reaction mixture was concentrated in vacuum and dried to give compound 06-103 (34.0 mg, 52.4 μmol, 93% yield). M+H⁺=610.4 (LCMS). ¹H NMR (MeOD, 400 MHz): δ 7.77 (br d, J=7.3 Hz, 1H), 7.65 (d, J=8.5 Hz, 1H), 7.60 (s, 1H), 7.58-7.54 (m, 1H), 7.52-7.42 (m, 4H), 7.33 (d, J=8.4 Hz, 1H), 7.16 (d, J=2.6 Hz, 1H), 6.96 (dd, J=2.8, 8.4 Hz, 1H), 4.55 (br d, J=18.4 Hz, 4H), 4.41 (t, J=6.7 Hz, 2H), 4.18 (s, 2H), 3.90-3.82 (m, 3H), 3.65 (br s, 8H), 2.99-2.91 (m, 2H), 2.81-2.75 (m, 3H), 2.30-2.19 (n, 2H).

### Example 36: Synthesis of 3-[5-[[4-[(2-chlorophenyl)methyl]piperazin-1-yl]methyl]-3-[2-[(dimethylamino)methyl]-4-methoxy-phenyl]indol-1-yl]propan-1-amine (06-102)

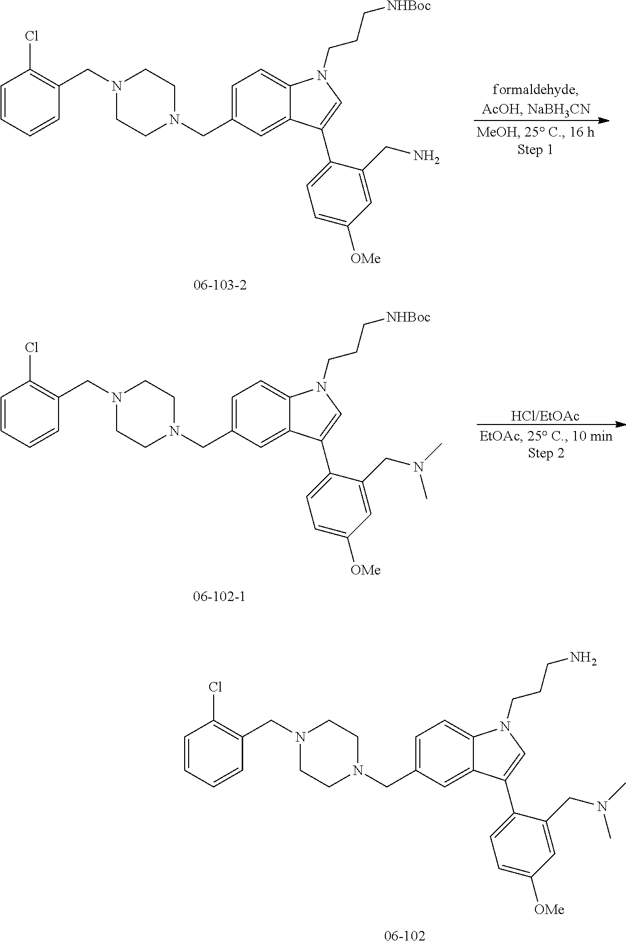 [SMILES: COc1ccc(-c2cn(CCCNC(=O)OC(C)(C)C)c3ccc(CN4CCN(Cc5ccccc5Cl)CC4)cc23)c(CN)c1 ; "formaldehyde, AcOH, NaBH3CN" ; COc1ccc(-c2cn(CCCNC(=O)OC(C)(C)C)c3ccc(CN4CCN(Cc5ccccc5Cl)CC4)cc23)c(CN(C)C)c1 ; "HCl/EtOAc" ; COc1ccc(-c2cn(CCCN)c3ccc(CN4CCN(Cc5ccccc5Cl)CC4)cc23)c(CN(C)C)c1]

### Step 1: tert-butyl N-[3-[5-[[4-[(2-chlorophenyl)methyl]piperazin-1-yl]methyl]-3-[2-[(dimethylamino)methyl]-4-methoxy-phenyl]indol-1-yl]propyl]carbamate (06-102-1)

To a stirred solution of tert-butyl N-[3-[3-[2-(aminomethyl)-4-methoxy-phenyl]-5-[[4-[(2-chlorophenyl)methyl]piperazin-1-yl]methyl]indol-1-yl]propyl]carbamate (260.0 mg, 411.2 μmol, 1.0 eq) in MeOH (10 mL) was added formaldehyde (100.1 mg, 1.23 mmol, 91.9 μL, 3.0 eq) and AcOH (37.0 mg, 616.9 μmol, 35.3 μL, 1.5 eq). After 4 h, NaBH₃CN (78.0 mg, 1.24 mmol, 3.0 eq) was added, and the reaction mixture was stirred at 25° C. for 12 h. The reaction mixture was poured into water (20 mL), and extracted with DCM (10 mL*4). The combined organic layers were washed with brine (20 mL), dried over anhydrous Na₂SO₄, filtered and concentrated under reduced pressure. The residue was purified by prep-HPLC (TFA condition) to give compound 06-102-1 (100 mg, TFA salt). M+H⁺=660.4 (LCMS).

### Step 2: 3-[5-[[4-[(2-chlorophenyl)methyl]piperazin-1-yl]methyl]-3-[2-[(dimethylamino)methyl]-4-methoxy-phenyl]indol-1-yl]propan-1-amine (06-102)

To a stirred solution of 06-102-1 (80.0 mg, 103.3 μmol, 1.0 eq, TFA salt) in EtOAc (1 mL) was added HCl/EtOAc (4 M, 1.00 mL, 38.7 eq) at 25° C. After 10 min, the reaction mixture was concentrated and dried under lyophilization to give compound 06-102 (34.6 mg, HCl salt). M+H⁺=560.4 (LCMS). ¹H NMR (MeOD, 400 MHz): δ 7.78 (br d, J=7.0 Hz, 1H), 7.69 (br d, J=8.2 Hz, 1H), 7.62 (s, 1H), 7.58-7.53 (m, 2H), 7.51-7.42 (m, 4H), 7.33 (s, 1H), 7.14 (br d, J=8.3 Hz, 1H), 4.55 (br d, J=5.0 Hz, 4H), 4.46 (br s, 4H), 3.92 (s, 3H), 3.66 (br s, 8H), 3.00 (br s, 2H), 2.65 (s, 6H), 2.28 (br s, 2H).

### Example 37: Synthesis of [2-[5-[[4-[(2-chlorophenyl)methyl]piperazin-1-yl]methyl]-1-(4-piperidyl) indol-3-yl]-5-(trifluoromethoxy)phenyl]methanamine (06-60)

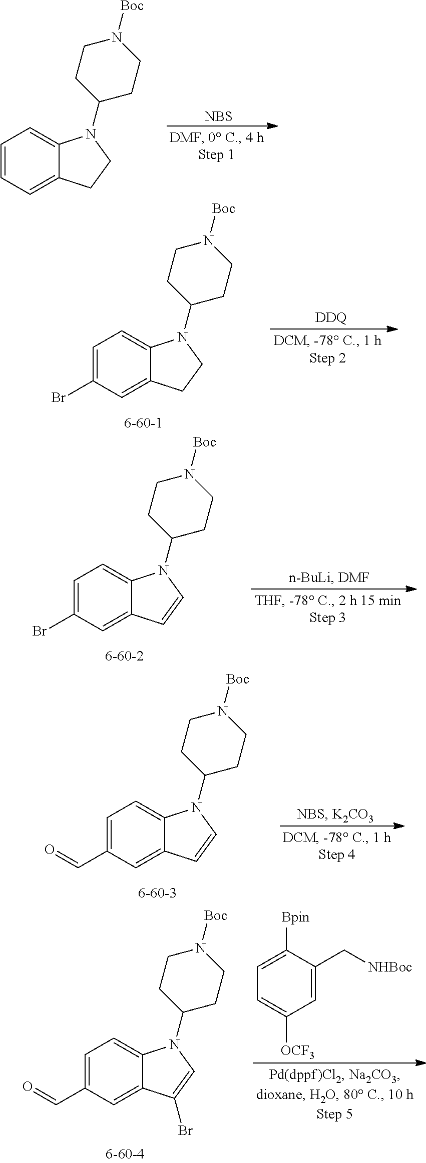

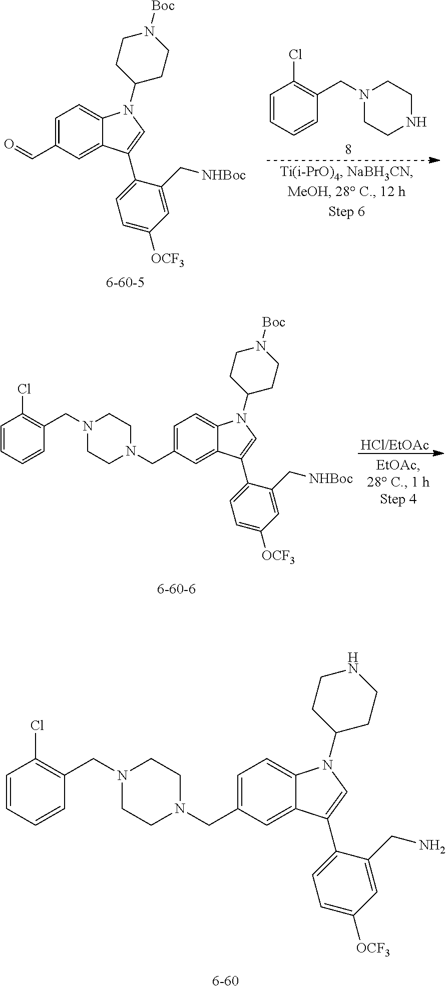 [SMILES: CC(C)(C)OC(=O)NCc1cc(OC(F)(F)F)ccc1-c1cn(C2CCN(C(=O)OC(C)(C)C)CC2)c2ccc(C=O)cc12 ; Clc1ccccc1CN1CCNCC1 ; CC(C)(C)OC(=O)NCc1cc(OC(F)(F)F)ccc1-c1cn(C2CCN(C(=O)OC(C)(C)C)CC2)c2ccc(CN3CCN(Cc4ccccc4Cl)CC3)cc12 ; "HCl/EtOAc" ; NCc1cc(OC(F)(F)F)ccc1-c1cn(C2CCNCC2)c2ccc(CN3CCN(Cc4ccccc4Cl)CC3)cc12]

### Step 1: tert-butyl 4-(5-bromoindolin-1-yl)piperidine-1-carboxylate (06-60-1)

To a mixture of tert-butyl 4-indolin-1-ylpiperidine-1-carboxylate (3.40 g, 11.24 mmol, 1.0 eq) in DMF (15 mL) at 0° C. was added NBS (2.00 g, 11.24 mmol, 1.0 eq) in DMF (15 mL). After 4 h at 0° C., the reaction mixture was poured into H₂O (45 mL) and extracted with EtOAc (35 mL*3). The organic layers were washed with brine (35 ml), dried over anhydrous Na₂SO₄, filtered and evaporated. The crude product was purified by column chromatography (SiO₂) to give compound 06-60-1 (3.40 g, 73% yield). M+H⁺=383.1 (LCMS). ¹H NMR (CDCl₃, 400 MHz): δ 7.06 (s, 2H), 6.21-6.17 (m, 1H), 3.41-3.32 (m, 1H), 3.31-3.24 (m, 2H), 2.89-2.82 (m, 2H), 2.74-2.63 (m, 2H), 1.76-1.64 (m, 2H), 1.51-1.44 (m, 2H), 1.40 (s, 9H), 0.84-0.74 (m, 2H).

### Step 2: tert-butyl 4-(5-bromoindol-1-yl)piperidine-1-carboxylate (06-60-2)

To a mixture of 06-60-1 (1.70 g, 4.46 mmol, 1.0 eq) in DCM (15 mL) at −78° C. was added DDQ (1.52 g, 6.69 mmol, 1.5 eq). After 1 h at −78° C., the reaction mixture was poured into saturated Na₂SO₃ solution (200 mL) and extract with DCM (40 mL*2). The organic layers were washed by brine (50 mL), dried over anhydrous Na₂SO₄, filtered and concentrated. The crude product was purified by silica gel column followed by reversed MPLC. The separated solution was adjusted to pH=8 by saturated NaHCO3 solution and evaporated to remove MeOH. The residue was extract by EtOAc (40 mL*3), dried over anhydrous Na₂SO₄, filtered and evaporated to give compound 06-60-2 (2.00 g, 55% yield). M+H⁺=403.0 (LCMS).

### Step 3: tert-butyl 4-(5-formylindol-1-yl)piperidine-1-carboxylate (06-60-3)

To a mixture of 06-60-2 (1.50 g, 3.95 mmol, 1.0 eq) in THF (15 mL) at −78° C., was added n-BuLi (2.5 M, 3.95 mL, 2.5 eq). After 15 min, DMF (346.9 mg, 4.75 mmol, 365.1 μL, 1.2 eq) was added. The reaction mixture was stirred at −78° C. for 2 h, poured into H₂O (35 mL) and extracted with EtOAc (25 mL*3). The organic layers were washed with brine (30 mL), filtered and concentrated to give the crude product. The crude product was purified by column chromatography (SiO₂) to give compound 06-60-3 (1.23 g). M+H⁺=329.1 (LCMS).

### Step 4: tert-butyl 4-(3-bromo-5-formyl-indol-1-yl)piperidine-1-carboxylate (06-60-4)

To a mixture of 06-60-3 (660.0 mg, 2.01 mmol, 1.0 eq) in DCM (10 mL) at −78° C. were added K₂CO₃ (416.6 mg, 3.01 mmol, 1.5 eq) and NBS (357.7 mg, 2.01 mmol, 1.0 eq). The reaction mixture was stirred at −78° C. for 1 h, poured into H₂O (35 mL) and extracted with DCM (20 mL*3). The organic layers were washed with brine (20 mL), dried over anhydrous Na₂SO₄, filtered and concentrated to give compound 06-60-4 (660.0 mg, 62% yield). M+H⁺=407.1 (LCMS).

### tert-butyl 4-[3-[2-[(tert-butoxycarbonylamino)methyl]-4-(trifluoromethoxy)phenyl]-5-formyl-indol-1-yl]piperidine-1-carboxylate (06-60-5)

M+H⁺=618.3 (LCMS).

### tert-butyl 4-[3-[2-[(tert-butoxycarbonylamino)methyl]-4-(trifluoromethoxy)phenyl]-5-[[4-[(2-chlorophenyl)methyl]piperazin-1-yl]methyl]indol-1-yl]piperidine-1-carboxylate tert-butyl (06-60-6)

M+H⁺=812.4 (LCMS).

### [2-[5-[[4-[(2-chlorophenyl)methyl]piperazin-1-yl]methyl]-1-(4-piperidyl)indol-3-yl]-5-(trifluoromethoxy)phenyl]methanamine (06-60)

M+H⁺=612.1 (LCMS). ¹H NMR (MeOD, 400 MHz): δ 7.85-7.81 (m, 1H), 7.80-7.76 (m, 1H), 7.75 (s, 2H), 7.68-7.64 (m, 1H), 7.63-7.61 (m, 1H), 7.59-7.55 (m, 1H), 7.55-7.50 (m, 2H), 7.49-7.43 (m, 2H), 4.58 (s, 4H), 4.32-4.26 (m, 2H), 3.76-3.59 (m, 10H), 3.46-3.34 (m, 3H), 2.54-2.33 (m, 4H).

The following compounds are synthesized according to similar procedures as described above for the preparation of 06-60.

| Comp | | | Mass | NMR (MeOD, |
|---|---|---|---|---|
| ID | Structure | Chemical Name | (M + H⁺) | 400 MHz) |
| 06-100 | 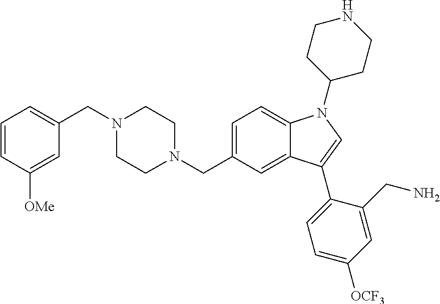 [SMILES: COc1cccc(CN2CCN(Cc3ccc4c(c3)c(-c3ccc(OC(F)(F)F)cc3CN)cn4C3CCNCC3)CC2)c1] | (2-(5-((4-(3- methoxybenzyl) piperazin-1-yl) methyl)-1- (piperidin-4-yl)- 1H-indol-3-yl)-5- (trifluoromethoxy) phenyl)methanamine dihydrochloride | Calc'd for C₃₄H₄₁F₃ N₅O₂: 608.3; Found: 608.4 | δ 7.81 (d, J = 8.6 Hz, 1H), 7.72 (s, 1H), 7.70 (s, 1H), 7.65 (s, 1H), 7.63 (s, 1H), 7.60 (d, J = 2.2 Hz, 1H), 7.51 (dd, J = 1.3, 8.6 Hz, 1H), 7.45 (dd, J = 1.3, 8.6 Hz, 1H), 7.38 (t, J = 7.9 Hz, 1H), 7.22 (d, J = 1.8 Hz, 1H), 7.13 (d, J = 7.5 Hz, 1H), 7.04 (dd, J = 1.9, 8.3 Hz, 1H), 4.97- 4.91 (m, 1H), 4.56 (s, 2H), 4.42 (br s, 2H), 4.27 (s, 2H), 3.84 (s, 3H), 3.74- 3.54 (m, 10H), 3.41-3.33 (m, 2H), 2.50-2.32 (m, 4H) |
| 06-101 | 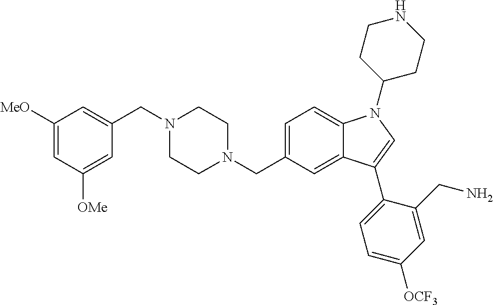 [SMILES: COc1cc(CN2CCN(Cc3ccc4c(c3)c(-c3ccc(OC(F)(F)F)cc3CN)cn4C3CCNCC3)CC2)cc(OC)c1] | (2-(5-((4-(3,5- dimethoxybenzyl) piperazin-1- yl)methyl)-1- (piperidin-4-yl)-1H- indol-3-yl)-5- (trifluoromethoxy) phenyl)methanamine dihydrochloride | Calc'd for C₃₅H₄₃F₃ N₅O₃: 638.3; Found: 638.4 | δ 9.73-9.59 (m, 1H), 6.64 (s, 1H), 6.48 (s, 1H), 5.19- 5.13 (m, 1H), 4.49-4.36 (m, 1H), 4.07-3.80 (m, 1H), 3.90 (br dd, J = 3.9, 14.1 Hz, 2H), 3.23- 3.11 (m, 4H), 3.06- 2.93 (m, 3H), 2.71-2.65 (m, 3H), 2.63-2.61 (m, 1H), 2.63- 2.61 (m, 1H), 1.81- 1.73 (m, 2H), 1.49-1.34 (m, 2H), 1.18 (br d, J = 6.9 Hz, 6H) |

### Example 38: Synthesis of N-[[2-[1-(3-aminopropyl)-5-[[4-[(2-chlorophenyl)methyl]piperazin-1-yl]methyl]indol-3-yl]-5-(trifluoromethoxy)phenyl]methyl]acetamide (06-95)

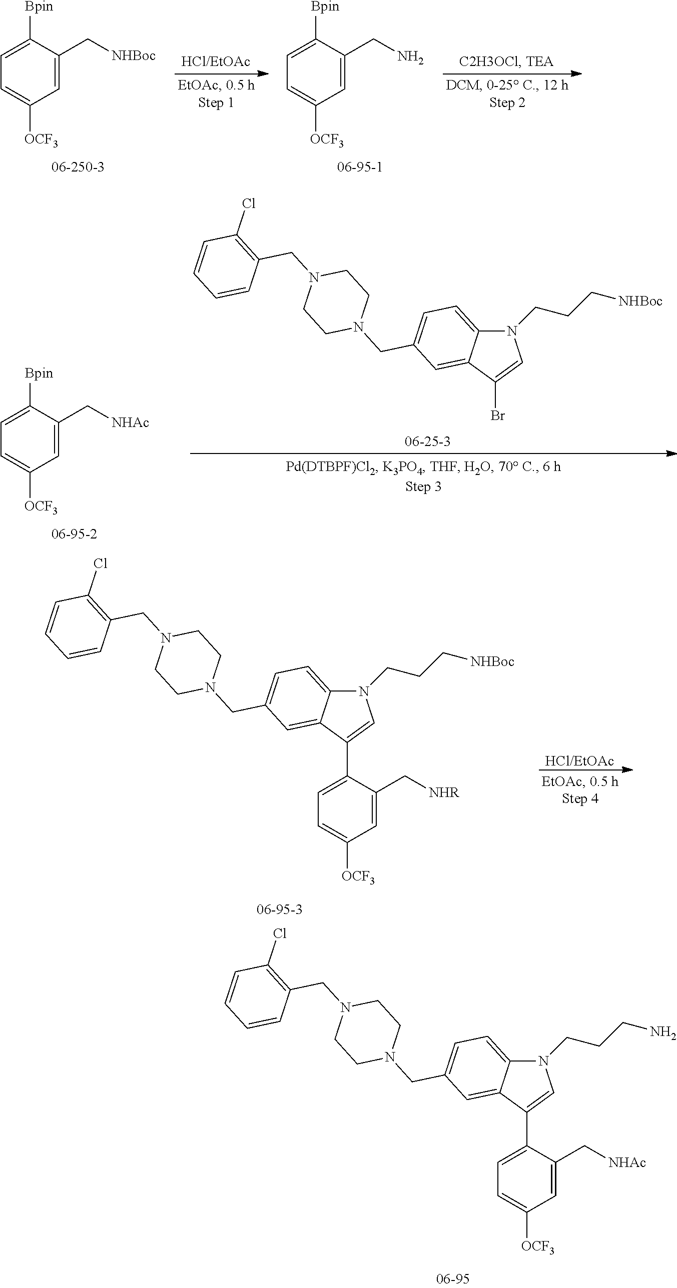

### Step 1: [2-(4,4,5,5-tetramethyl-1,3,2-dioxaborolan-2-yl)-5-(trifluoromethoxy)phenyl]methanamine (06-95-1)

To a solution of tert-butyl N-[[2-(4,4,5,5-tetramethyl-1,3,2-dioxaborolan-2-yl)-5-(trifluoromethoxy)phenyl]methyl]carbamate (1.50 g, 3.60 mmol, 1.0 eq) in EtOAc (5 mL) was added HCl/EtOAc (4 M, 5.00 mL, 5.6 eq). The mixture was stirred at 25° C. for 30 min and poured into H₂O (80 mL). The aqueous phase was adjusted to pH 8 with solid NaHCO₃, and extracted with dichloromethane (30 mL*6). The combined organic layers were washed with brine (80 mL), dried over anhydrous Na₂SO₄, filtered and concentrated in vacuum to give compound 06-95-1 (900.0 mg, crude). M−102+H⁺=236.2 (LCMS).

### Step 2:N-[[2-(4,4,5,5-tetramethyl-1,3,2-dioxaborolan-2-yl)-5-(trifluoromethoxy)phenyl]methyl]acetamide (06-95-2)

To a solution of 06-95-1 (280.0 mg, 883.0 μmol, 1.0 eq) in DCM (5 mL) was added TEA (180.0 mg, 1.78 mmol, 246.6 μL, 2.0 eq) and acetyl chloride (70.0 mg, 891.7 μmol, 63.6 μL, 1.01 eq) at 0° C. The mixture was stirred at 0° C. for 1 h, warmed to 25° C. and stirred for another 11 h. The reaction mixture was poured into H₂O (150 mL) and extracted with DCM (30 mL*5). The combined organic layers were washed with brine (80 mL), dried over anhydrous Na₂SO₄, filtered and concentrated in vacuum to give compound 06-95-2 (250 mg, crude). M+H⁺=360.2 (LCMS).

Steps 3 and 4 are carried out according to similar procedures as described in steps 5 and 6 in the synthesis of 06-25.

### tert-butyl N-[3-[3-[2-(acetamidomethyl)-4-(trifluoromethoxy)phenyl]-5-[[4-[(2-chlorophenyl)methyl]piperazin-1-yl]methyl]indol-1-yl]propyl]carbamate (06-95-3)

M+H⁺=728.3 (LCMS).

### N-[[2-[1-(3-aminopropyl)-5-[[4-[(2-chlorophenyl)methyl]piperazin-1-yl]methyl]indol-3-yl]-5-(trifluoromethoxy)phenyl]methyl]acetamide (06-95)

M+H⁺=628.4 (LCMS). ¹H NMR (MeOD, 400 MHz): δ 7.82-7.78 (m, 1H), 7.70-7.65 (m, 2H), 7.60-7.56 (m, 1H), 7.54-7.51 (m, 1H), 7.51-7.46 (m, 4H), 7.34 (s, 1H), 7.28 (br d, J=9.2 Hz, 1H), 4.66 (s, 2H), 4.57 (s, 2H), 4.43 (t, J=7.0 Hz, 2H), 4.37 (s, 2H), 3.83-3.64 (m, 8H), 3.00-2.95 (m, 2H), 2.30-2.22 (m, 2H), 1.90 (s, 3H).

### Example 39: Synthesis of 3-(3-(2-(aminomethyl)-4-(trifluoromethoxy)phenyl)-5-((4-(2-chlorobenzyl)piperazin-1-yl)sulfonyl)-1H-indol-1-yl)propan-1-amine (06-63)

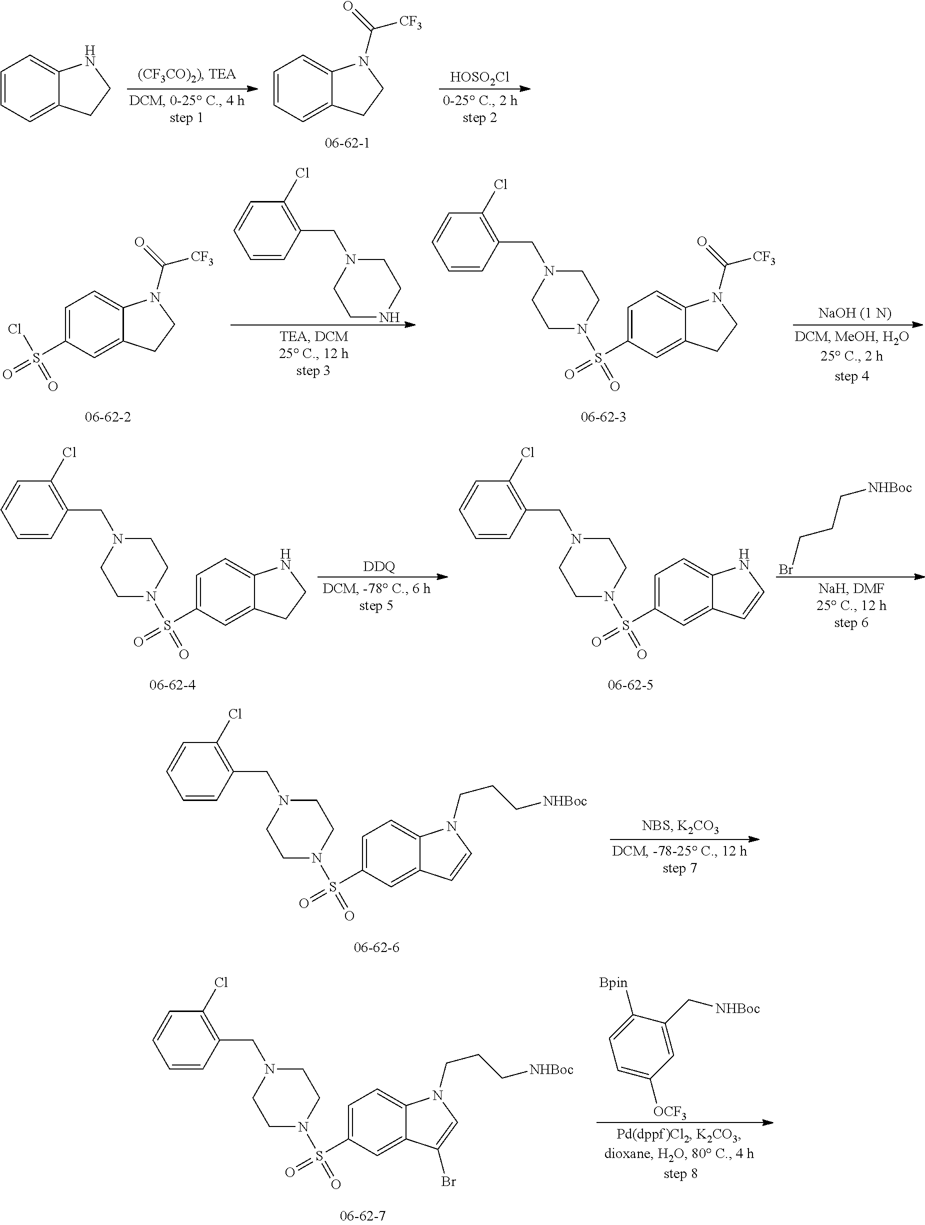

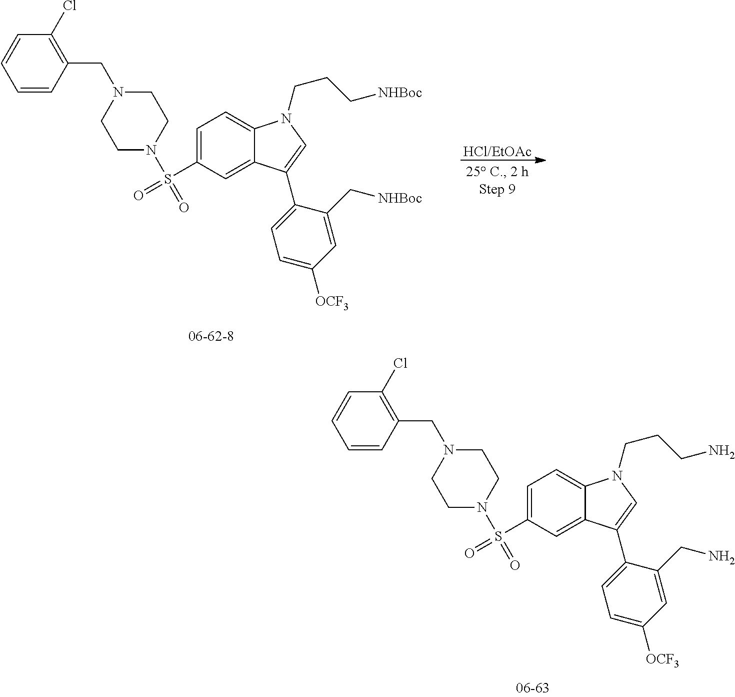 [SMILES: CC(C)(C)OC(=O)NCCCn1cc(-c2ccc(OC(F)(F)F)cc2CNC(=O)OC(C)(C)C)c2cc(S(=O)(=O)N3CCN(Cc4ccccc4Cl)CC3)ccc21 ; "HCl/EtOAc" ; NCCCn1cc(-c2ccc(OC(F)(F)F)cc2CN)c2cc(S(=O)(=O)N3CCN(Cc4ccccc4Cl)CC3)ccc21]

### Step 1: 2,2,2-trifluoro-1-(indolin-1-yl)ethanone (06-63-1)

To a solution of indoline (20 g, 167.84 mmol, 18.87 mL, 1.0 eq) in DCM (300 mL) was added TEA (50.95 g, 503.52 mmol, 69.79 mL, 3 eq) and (2,2,2-trifluoroacetyl) 2,2,2-trifluoroacetate (70.50 g, 335.68 mmol, 46.69 mL, 2 eq) dropwise at 0° C. The mixture was stirred at 25° C. for 4 h, poured into DCM (200 mL) and H₂O (300 mL). The organic phase was separated, washed with brine (100 mL*3), dried over anhydrous Na₂SO₄, filtered and concentrated under reduced pressure to give a residue. The crude product was purified by column (SiO₂) to give compound 06-63-1 (25 g, 64% yield). M+H⁺=216.1 (LCMS).

### Step 2: 1-(2,2,2-trifluoroacetyl)indoline-5-sulfonyl chloride (06-63-2)

Compound 06-63-1 (5 g, 23.24 mmol, 1 eq) was added to HSO₃Cl (17.5 g, 150.2 mmol, 10 mL, 6.5 eq) at 0° C. After 2 h at 25° C., the mixture was pour into the ice water and filtered. The solid collected was dried to give compound 06-63-2 (4.2 g, 55% yield). M+H⁺=314.1 (LCMS).

### Step 3: 1-(5-((4-(2-chlorobenzyl)piperazin-1-yl)sulfonyl)indolin-1-yl)-2,2,2-trifluoro ethanone (06-63-3)

To a solution of 06-63-2 (1.8 g, 5.74 mmol, 1 eq) and 1-[(2-chlorophenyl)methyl]piperazine (1.21 g, 5.74 mmol, 1 eq) in DCM (50 mL) was added TEA (1.74 g, 17.22 mmol, 2.39 mL, 3 eq). The mixture was stirred at 25° C. for 12 h, and partitioned between DCM (100 mL) and H₂O (100 mL). The organic phase was washed with brine (30 mL*3), dried over anhydrous Na₂SO₄, filtered and concentrated under reduced pressure to give compound 06-63-3 (crude 3.3 g). M+H⁺=488.2 (LCMS).

### Step 4: 5-((4-(2-chlorobenzyl)piperazin-1-yl)sulfonyl)indoline (06-63-4)

To a solution of 06-63-3 (3.3 g, 6.76 mmol, 1 eq) in MeOH (10 mL) and DCM (20 mL) was added NaOH (1 M, 60 mL, 8.88 eq). The mixture was stirred at 25° C. for 2 h, and concentrated under reduced pressure to remove MeOH and DCM. The residue was extracted with EtOAc (20 mL*3). The combined organic layers were washed with brine (10 mL*3), dried over anhydrous Na₂SO₄, filtered and concentrated under reduced pressure to give a residue which was purified by column (SiO₂) to give compound 06-63-4 (1.5 g, 39% yield). M+H⁺=392.2 (LCMS).

### Step 5: 5-((4-(2-chlorobenzyl)piperazin-1-yl)sulfonyl)-1H-indole (06-63-5)

To a solution of 06-63-4 (500 mg, 1.28 mmol, 1 eq) in DCM (10 mL) was added DDQ (434.4 mg, 1.91 mmol, 1.5 eq) at −78° C. The mixture was stirred at −78° C. for 6 h, warmed to rt, quenched with the addition of sat. Na₂SO₃ (30 mL), and extracted with DCM (20 mL*3). The combined organic layers were washed with brine (15 mL*3), dried over anhydrous Na₂SO₄, filtered and concentrated under reduced pressure. The residue was purified by column (SiO₂) to give compound 06-63-5 (420 mg, 59% yield). M+H⁺=390.1 (LCMS).

### Step 6: tert-butyl(3-(5-((4-(2-chlorobenzyl)piperazin-1-yl)sulfonyl)-1H-indol-1-yl)propyl) carbamate (06-63-6)

To a solution of 06-63-5 (370 mg, 948.96 μmol, 1 eq) in DMF (10 mL) was added NaH (94.8 mg, 2.37 mmol, 2.5 eq). After 30 min, tert-butyl (3-bromopropyl)carbamate (271.16 mg, 1.14 mmol, 1.20 eq) was added, and the resulting mixture was stirred at 25° C. for 11.5 h. The reaction mixture was quenched carefully with H2O (10 mL), and diluted with EtOAc (30 mL) and H2O (20 mL). The organic phase was washed with brine (10 mL*3), dried over anhydrous Na2SO4, filtered and concentrated under reduced pressure. The residue was purified by prep-TLC to give compound 06-63-6 (310 mg, 77% yield). M+H+=547.3 (LCMS).

### Step 7: tert-butyl(3-(3-bromo-5-((4-(2-chlorobenzyl)piperazin-1-yl)sulfonyl)-1H-indol-1-yl)propyl)carbamate (06-63-7)

To a solution of 06-63-6 (310 mg, 436.3 μmol, 1.0 eq) in DCM (10 mL) was added K₂CO₃ (150.75 mg, 1.09 mmol, 2.5 eq) and NBS (85.42 mg, 479.92 μmol, 1.1 eq) at −78° C. After 12 h at 25° C., the reaction mixture was diluted with DCM (20 mL) and H₂O (20 mL). The organic phase was washed with brine (10 mL*3), dried over anhydrous Na₂SO₄, filtered and concentrated under reduced pressure. The residue was purified by prep-TLC to give compound 06-63-7 (180 mg, 32% yield). M+H⁺=627.3 (LCMS).

### 3-(3-(2-(aminomethyl)-4-(trifluoromethoxy)phenyl)-5-((4-(2-chlorobenzyl)piperazin-1-yl)sulfonyl)-1H-indol-1-yl)propan-1-amine (06-63)

M+H⁺=636.3 (LCMS). ¹H NMR (MeOD, 400 MHz): δ 8.51 (br s, 1H), 7.80 (dd, J=3.4, 5.0 Hz, 2H), 7.68-7.64 (m, 2H), 7.59-7.54 (m, 2H), 7.41 (br d, J=8.4 Hz, 1H), 7.34 (dt, J=3.6, 5.3 Hz, 2H), 7.21 (dd, J=3.5, 5.7 Hz, 2H), 4.48 (br t, J=6.9 Hz, 2H), 4.06 (s, 2H), 3.61 (s, 2H), 2.99-2.94 (m, 6H), 2.56 (br t, J=4.6 Hz, 4H), 2.30-2.24 (m, 2H).

The following compounds are synthesized according to similar procedures as described above for the preparation of 06-63.

| Comp | | | Mass | NMR (MeOD, 400 |
|---|---|---|---|---|
| ID | Structure | Chemical Name | (M + H⁺) | MHz) |
| 04-80 | 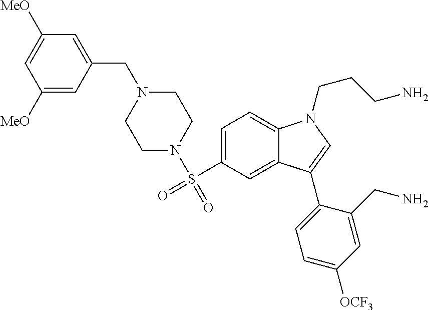 [SMILES: COc1cc(CN2CCN(S(=O)(=O)c3ccc4c(c3)c(-c3ccc(OC(F)(F)F)cc3CN)cn4CCCN)CC2)cc(OC)c1] | 3-(3-(2- (aminomethyl)-4- (trifluoromethoxy) phenyl)-5-((4-(3,5- dimethoxybenzyl) piperazin-1-yl) sulfonyl)-1H-indol- 1-yl)propan-1- amine triformate | Calc'd for C₃₂H₃₉F₃ N₅O₅S: 662.3; Found: 662.3 | δ 8.42 (brs, 3 H) 7.82-7.84 (m, 2 H) 7.69-7.71 (m, 2 H) 7.60-7.63 (m, 2 H) 7.48-7.50 (m, 1 H) 6.43 (d, J = 2.4 Hz 2 H) 6.36-6.37 (m, 1 H) 4.49-4.52 (m, 2 H) 4.21 (s, 2 H) 3.73 (s, 6 H) 3.46 s, 2 H) 3.00-3.04 (m, 6 H) 2.55 (m, 4 H) 2.28-2.32 (m, 2 H) |
| 08-10 | 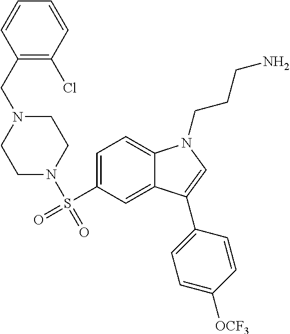 [SMILES: NCCCn1cc(-c2ccc(OC(F)(F)F)cc2)c2cc(S(=O)(=O)N3CCN(Cc4ccccc4Cl)CC3)ccc21] | 3-(5-((4-(2- chlorobenzyl) piperazin-1-yl) sulfonyl)-3-(4- (trifluoromethoxy) phenyl)-1H- indol-1-yl) propan-1-amine formate | Calc'd for C₂₉H₃₁Cl F₃N₄O₃S: 607.2; Found: 607.3 | δ 8.54 (s, 1 H) 8.26 (s, 1 H) 7.73-7.79 (m, 4 H) 7.66-7.68 (m, 1 H) 7.40-7.42 (m, 4 H) 7.19-7.22 (m, 2 H) 4.46-4.49 (m, 2 H) 3.61 (s, 2 H) 2.96-3.02 (m, 6 H) 2.57-2.69 (m, 4 H) 2.25-2.31 (m, 2 H) |

### Example 40: Synthesis of 2-[1-(3-aminopropyl)-5-[[4-[(2-chlorophenyl)methyl]piperazin-1-yl]methyl]indol-3-yl]-5-(trifluoromethoxy)aniline (06-59)

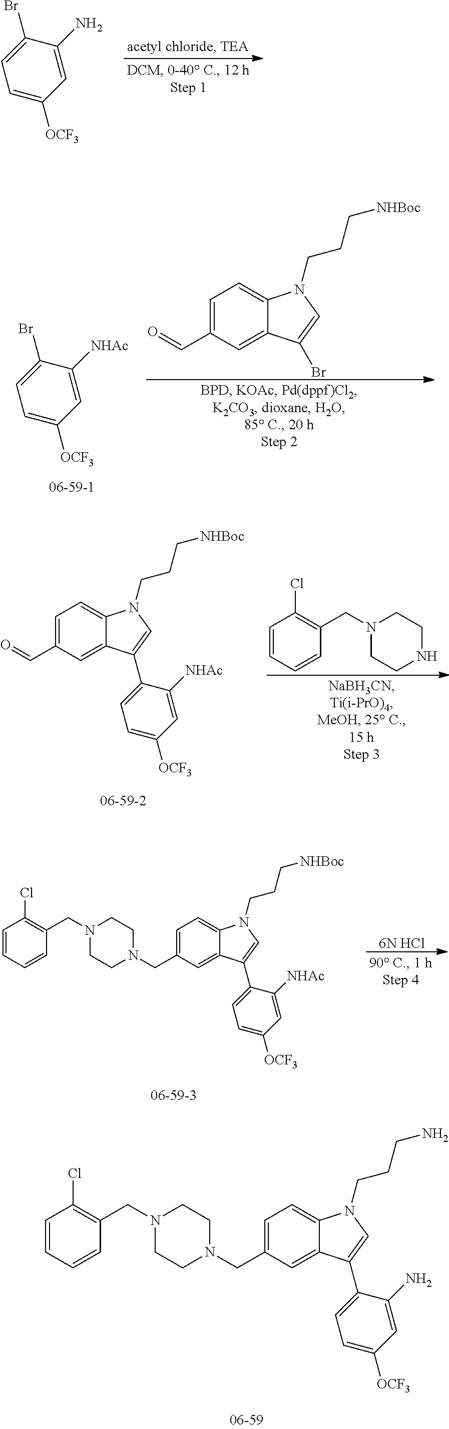

### Step 1: N-[2-bromo-5-(trifluoromethoxy)phenyl]acetamide (06-59-1)

To a solution of 2-bromo-5-(trifluoromethoxy)aniline (1.00 g, 3.91 mmol, 1.0 eq) in DCM (10 mL) was added TEA (800.0 mg, 7.91 mmol, 1.10 mL, 2.02 eq) and acetyl chloride (620.0 mg, 7.90 mmol, 563.6 μL, 2.02 eq) at 0° C. After 5 h at 0° C., the mixture was warmed to 40° C. and stirred for another 7 h under N₂. The reaction mixture was poured into H₂O (150 mL), and extracted with EtOAc (50 mL*3). The combined organic layers were washed with brine (80 mL), dried over anhydrous Na₂SO₄, filtered and the filtrate was concentrated in vacuum. The residue was purified by column chromatography (SiO₂) to give compound 06-59-1 (900.0 mg, 2.33 mmol, 60% yield). M+H⁺=297.0 (LCMS). ¹H NMR (CDCl₃, 400 MHz): δ 8.41 (br s, 1H), 7.55 (d, J=8.8 Hz, 1H), 6.88 (dd, J=1.8, 8.8 Hz, 1H), 2.27 (s, 3H)

### Step 2: tert-butyl N-[3-[3-[2-acetamido-4-(trifluoromethoxy)phenyl]-5-formyl-indol-1-yl]propyl]carbamate (06-59-2)

To a solution of 06-59-1 (800.0 mg, 2.68 mmol, 1.0 eq) and 4,4,5,5-tetramethyl-2-(4,4,5,5-tetramethyl-1,3,2-dioxaborolan-2-yl)-1,3,2-dioxaborolane (1.00 g, 3.94 mmol, 1.47 eq) in dioxane (10 mL) was added KOAc (640.0 mg, 6.52 mmol, 2.43 eq) and Pd(dppf)Cl₂ (100.0 mg, 136.7 μmol, 0.05 eq). The mixture was stirred at 85° C. for 8 h under N₂. Then tert-butyl N-[3-(3-bromo-5-formyl-indol-1-yl)propyl]carbamate (820.0 mg, 2.15 mmol, 0.8 eq), Pd(dppf)Cl₂ (100.0 mg, 136.7 μmol, 0.05 eq), K₂CO₃ (800.0 mg, 5.79 mmol, 2.2 eq) and H₂O (1 mL) were added. The resulting mixture was stirred at 85° C. for 12 h under N₂. The reaction mixture was poured into H₂O (150 mL) and extracted with EtOAc (50 mL*3). The combined organic layers were washed with brine (80 mL), dried over anhydrous Na₂SO₄, filtered and the filtrate was concentrated in vacuum. The residue was purified by column chromatography (SiO₂) to give compound 06-59-2 (800 mg, 51% yield). ¹H NMR (CDCl₃, 400 MHz): δ 10.00 (s, 1H), 8.40 (br s, 1H), 7.99 (s, 1H), 7.91-7.83 (m, 1H), 7.65 (br s, 1H), 7.52 (d, J=8.7 Hz, 1H), 7.41-7.33 (m, 2H), 7.06 (br d, J=7.7 Hz, 1H), 4.33 (br t, J=6.6 Hz, 2H), 3.21 (q, J=6.4 Hz, 2H), 2.13 (br t, J=6.5 Hz, 2H), 1.98 (s, 3H), 1.42 (br s, 9H)

### Step 3: tert-butyl N-[3-3-[3-[2-acetamido-4-(trifluoromethoxy)phenyl]-5-[[4-[(2-chlorophenyl)methyl]piperazin-1-yl]methyl]indol-1-yl]propyl]carbamate (06-59-3)

To a solution of 06-59-2 (700.0 mg, 1.08 mmol, 1.0 eq) and 1-[(2-chlorophenyl)methyl]piperazine (339.1 mg, 1.61 mmol, 1.5 eq) in MeOH (8.00 mL) was added Ti(i-PrO)₄ (310.0 mg, 1.09 mmol, 322.9 μL, 1.01 eq). After 3 h, NaBH₃CN (150.0 mg, 2.39 mmol, 2.21 eq) was added and the resulting mixture was stirred at 25° C. for another 12 h under N₂. The reaction mixture was poured into H₂O (100 mL), filtered and the filter residue was washed with DCM (50 mL*3). The mixture was extracted with DCM (50 mL*3). The combined organic layers were washed with brine (120 mL), dried over anhydrous Na₂SO₄, filtered and the filtrate was concentrated in vacuum. The residue was purified by column chromatography (SiO₂) to give compound 06-59-3 (500 mg). M+H⁺=714.3 (LCMS). ¹H NMR (CDCl₃, 400 MHz): δ 10.00 (s, 1H), 7.98 (br s, 1H), 7.85 (br d, J=8.4 Hz, 1H), 7.48 (br d, J=8.3 Hz, 1H), 7.43 (br d, J=8.4 Hz, 1H), 7.22 (br d, J=13.4 Hz, 2H), 7.19 (br s, 1H), 4.51-4.37 (m, 2H), 4.29 (br s, 2H), 3.21 (br d, J=5.6 Hz, 3H), 2.73 (br s, 2H), 2.16-2.08 (m, 2H), 1.45 (s, 18H)

### Step 4: 2-[1-(3-aminopropyl)-5-[[4-[(2-chlorophenyl)methyl]piperazin-1-yl]methyl]indol-3-yl]-5-(trifluoromethoxy)aniline (06-59)

A solution of 06-59-3 (260.0 mg, 364.0 μmol, 1.0 eq) in HCl (6 M, 8.0 mL) was stirred at 90° C. for 1 h. The reaction mixture was cooled to rt, poured into saturated sodium carbonate solution (100 mL, pH=9-10), and extracted with DCM (30 mL*8). The combined organic layers were washed with brine (80 mL), dried over anhydrous Na₂SO₄, filtered and the filtrate was concentrated in vacuum to give a residue, which was purified by prep-HPLC (FA condition) to give compound 06-59 (95.8 mg, FA salt, 39% yield). M+H⁺=572.3 (LCMS). ¹H NMR (MeOD, 400 MHz): δ 8.51 (s, 1H), 7.58-7.54 (m, 2H), 7.48-7.44 (m, 2H), 7.40-7.36 (m, 1H), 7.31 (dd, J=1.4, 8.7 Hz, 1H), 7.23 (s, 1H), 7.28-7.22 (m, 2H), 6.75 (d, J=1.3 Hz, 1H), 6.64-6.59 (m, 1H), 4.39 (t, J=6.8 Hz, 2H), 3.99 (s, 2H), 3.69 (s, 2H), 2.97-2.93 (m, 2H), 2.67 (br s, 4H), 2.87 (br s, 4H), 2.28-2.20 (m, 2H).

### Example 41: Synthesis of 3-[5-[[4-[(2-chlorophenyl)methyl]piperazin-1-yl]methyl]-3-[2-(methylaminomethyl)-4-(trifluoromethoxy)phenyl]indol-1-yl]propan-1-amine (06-56)

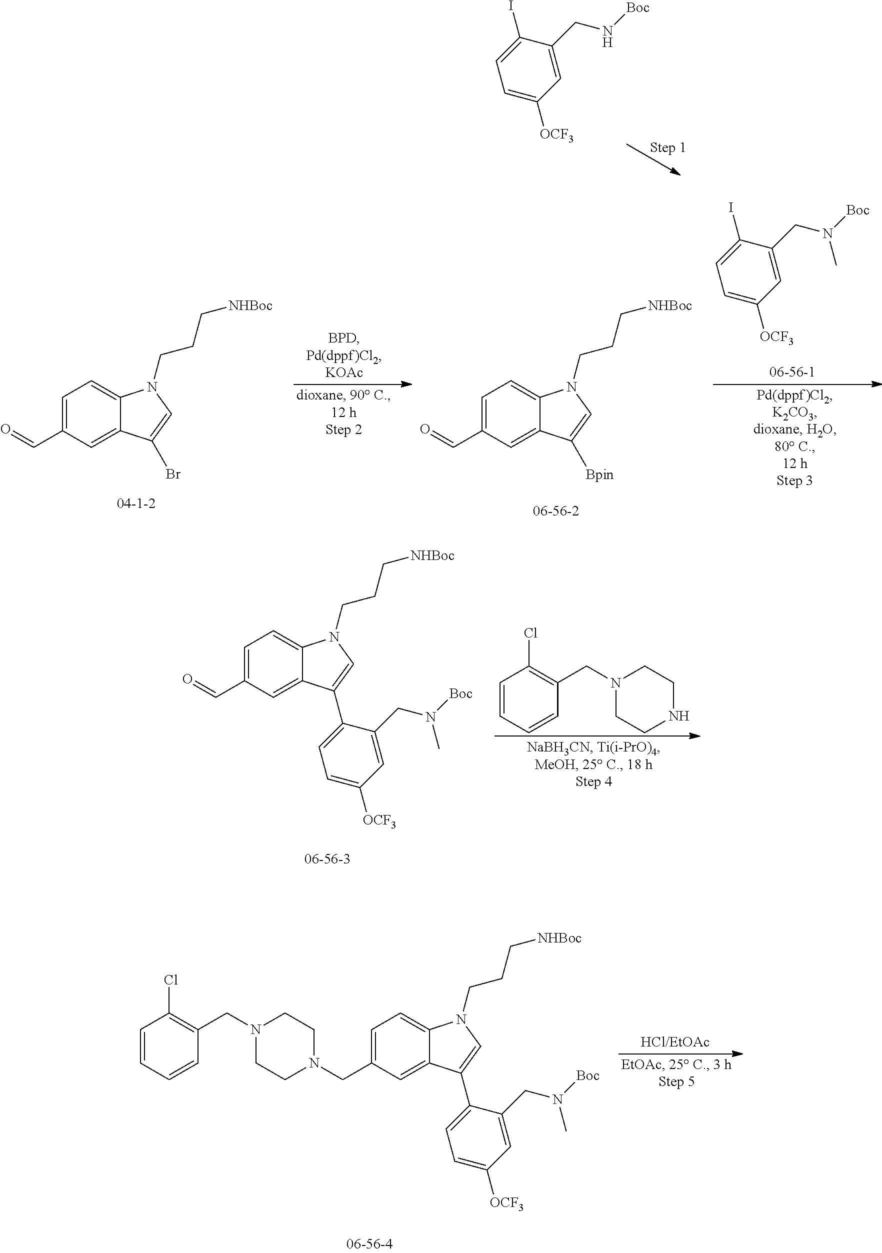

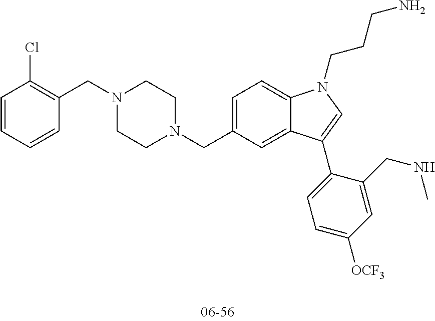 [SMILES: CNCc1cc(OC(F)(F)F)ccc1-c1cn(CCCN)c2ccc(CN3CCN(Cc4ccccc4Cl)CC3)cc12]

### Step 1: tert-butyl N-[[2-iodo-5-(trifluoromethoxy)phenyl]methyl]-N-methyl-carbamate (6-56-1)

To a solution of tert-butyl N-[[2-iodo-5-(trifluoromethoxy)phenyl]methyl]carbamate (400.0 mg, 958.9 μmol, 1.0 eq) in DMF (8 mL) was added NaH (100.0 mg, 2.50 mmol, 2.6 eq) at −78° C. After 30 min, iodomethane (1.48 g, 10.4 mmol, 650.1 μL, 10.9 eq) was added dropwise at −78° C. The resulting mixture was stirred at −78° C. for 0.5 h under N₂, warmed to 0° C. and stirred for another 1 h. The reaction mixture was poured into H₂O (100 mL) and extracted with EtOAc (30 mL*3). The combined organic layers were washed with brine (50 mL), dried over anhydrous Na₂SO₄, filtered and the filtrate was concentrated in vacuum to give a residue. The residue was purified by column chromatography (SiO₂) to give compound 06-56-1 (420.0 mg, 896.1 μmol, 94% yield). M−55+H⁺: 376.0 (LCMS). ¹H NMR (CDCl₃, 400 MHz): δ 7.96 (d, J=8.4 Hz, 1H), 7.04-6.90 (m, 2H), 4.43 (s, 2H), 3.37-3.24 (m, 1H), 2.92 (br s, 3H), 1.52-1.36 (m, 8H)

### Step 2: tert-butyl N-[3-[5-formyl-3-(4,4,5,5-tetramethyl-1,3,2-dioxaborolan-2-yl)indol-1-yl]propyl]carbamate (6-56-2)

To a solution of tert-butyl N-[3-(3-bromo-5-formyl-indol-1-yl)propyl]carbamate (2.00 g, 5.25 mmol, 1.0 eq) and 4,4,5,5-tetramethyl-2-(4,4,5,5-tetramethyl-1,3,2-dioxaborolan-2-yl)-1,3,2-dioxaborolane (1.40 g, 5.51 mmol, 1.05 eq) in dioxane (25 mL) were added KOAc (1.10 g, 11.2 mmol, 2.14 eq) and Pd(dppf)Cl₂ (345.7 mg, 472.5 μmol, 0.09 eq). The mixture was stirred at 90° C. for 12 h under N₂, poured into H₂O (150 mL) and extracted with EtOAc (50 mL*3). The combined organic layers were washed with brine (80 mL), dried over anhydrous Na₂SO₄, filtered and the filtrate was concentrated in vacuum to give a residue which was purified by column chromatography (SiO₂) to give compound 06-56-2 (2.00 g, 824.6 μmol, 16% yield). M−55+H⁺: 373.1 (LCMS).

### Step 3:tert-butyl N-[[2-[1-[3-(tert-butoxycarbonylamino)propyl]-5-formyl-indol-3-yl]-5-(trifluoromethoxy)phenyl]methyl]-N-methyl-carbamate (6-56-3)

To a mixture of 06-56-2 (2.00 g, 824.60 μmol, 1.0 eq) and 06-56-1 (390.0 mg, 832.9 μmol, 1.01 eq) in dioxane (20.00 mL) and H₂O (2.00 mL) was added K₂CO₃ (230.0 mg, 1.66 mmol, 2.02 eq) and Pd(dppf)Cl₂ (50.0 mg, 68.3 μmol, 0.08 eq). The mixture was stirred at 80° C. for 12 h under N₂, poured into H₂O (150 mL) and extracted with EtOAc (50 mL*3). The combined organic layers were washed with brine (80 mL), dried over anhydrous Na₂SO₄, filtered and the filtrate was concentrated. The residue was purified by column chromatography (SiO₂) to give compound 06-56-3 (280.00 mg, 374.48 μmol, 45% yield). M−100+H⁺=506.2 (LCMS). ¹H NMR (CDCl₃, 400 MHz): δ 10.00 (s, 1H), 7.98 (br s, 1H), 7.85 (br d, J=8.4 Hz, 1H), 7.48 (br d, J=8.3 Hz, 1H), 7.43 (br d, J=8.4 Hz, 1H), 7.22 (br d, J=13.4 Hz, 2H), 7.19 (br s, 1H), 4.51-4.37 (m, 2H), 4.29 (br s, 2H), 3.21 (br d, J=5.6 Hz, 3H), 2.73 (br s, 2H), 2.16-2.08 (m, 2H), 1.45 (s, 18H).

### 3-[5-[[4-[(2-chlorophenyl)methyl]piperazin-1-yl]methyl]-3-[2-(methylaminomethyl)-4-(trifluoromethoxy)phenyl]indol-1-yl]propan-1-amine (06-56)

M+H⁺=600.2 (LCMS). ¹H NMR (MeOD, 400 MHz): δ 7.77 (dd, J=1.7, 7.5 Hz, 1H), 7.73-7.69 (m, 2H), 7.68-7.63 (m, 3H), 7.57-7.54 (m, 1H), 7.52-7.48 (m, 2H), 7.48-7.41 (m, 2H), 4.56 (s, 4H), 4.47 (t, J=7.0 Hz, 2H), 4.38 (s, 2H), 3.74-3.54 (m, 8H), 3.05-2.98 (m, 2H), 2.58 (s, 3H), 2.29 (quin, J=7.4 Hz, 2H).

### Example 42: Synthesis of 2-[5-[[4-[(2,6-dichlorophenyl)methyl]piperazin-1-yl]methyl]-3-[4-(trifluoromethoxy)phenyl]indol-1-yl]ethanamine (07-2)

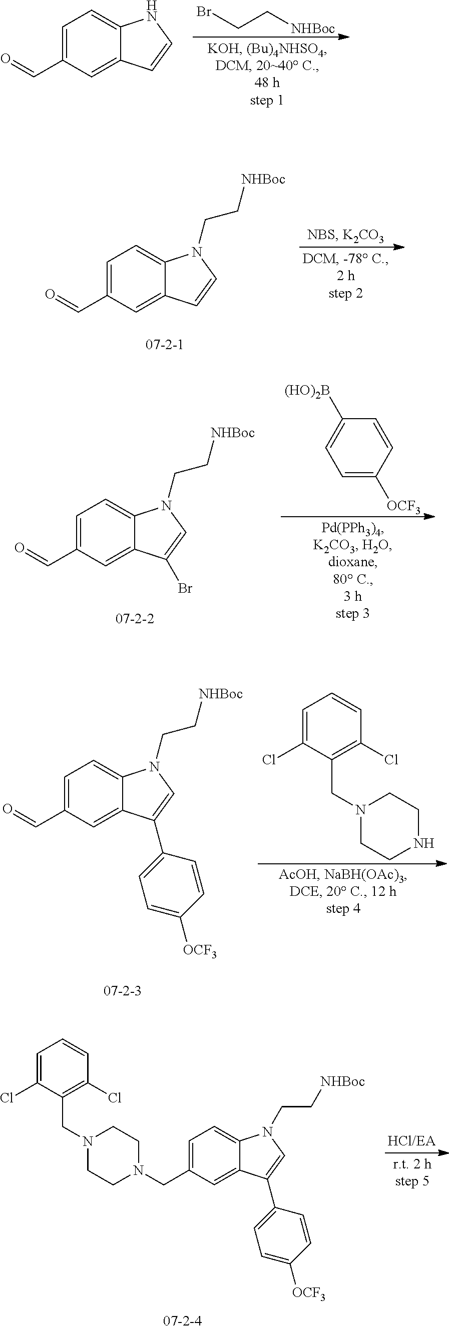

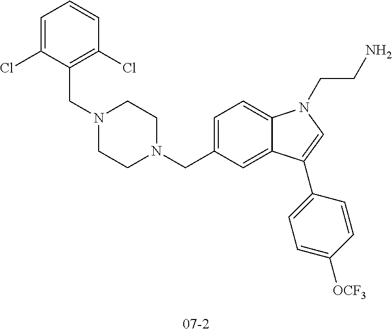 [SMILES: NCCn1cc(-c2ccc(OC(F)(F)F)cc2)c2cc(CN3CCN(Cc4c(Cl)cccc4Cl)CC3)ccc21]

### Step 1: tert-butyl N-[2-(5-formylindol-1-yl)ethyl]carbamate (07-2-1)

To a solution of 1H-indole-5-carbaldehyde (15.0 g, 103.3 mmol, 1.0 eq) in DCM (200 mL) was added tetrabutylammonium hydrogen sulfate (35.0 g, 103.3 mmol, 1.0 eq) and KOH (14.5 g, 258.3 mmol, 2.5 eq). The mixture was stirred at 20° C. for 12 h followed by heating at 40° C. for 36 h. The reaction mixture was poured into water (300 mL) and extracted with DCM (200 mL*3). The combined organic layers were dried over anhydrous Na₂SO₄ and concentrated under reduced pressure. The residue was purified by column chromatography (SiO₂) to give compound 07-2-1 (10.3 g, 31%). M+H⁺=289.2 (LCMS). ¹H NMR (CDCl₃, 400 MHz): δ 10.03 (d, J=4.4 Hz, 1H), 8.15 (s, 1H), 7.78 (d, J=8.4 Hz, 1H), 7.44 (d, J=8.4 Hz, 1H), 6.68 (d, J=3.2 Hz, 1H), 4.32 (d, J=4.8 Hz, 2H), 3.525-3.480 (m, 2H), 1.43 (s, 9H).

### Step 2: tert-butyl N-[2-(3-bromo-5-formyl-indol-1-yl)ethyl]carbamate (07-2-2)

To a solution of compound 07-2-1 (1.00 g, 3.47 mmol, 1.0 eq) in DCM (10 mL) was added K₂CO₃ (718.9 mg, 5.20 mmol, 1.5 eq) and NBS (617.6 mg, 3.47 mmol, 1.0 eq). The mixture was stirred at −78° C. for 2 h, poured into H₂O (50 mL) and extracted with DCM (100 mL*3). The combined organic layers were dried over anhydrous Na₂SO₄, filtered, and concentrated under reduced pressure to give compound 07-2-2 (1.28 g, crude), which was used into the next step without further purification. M+H⁺=367.1 (LCMS).

### Step 3: tert-butyl N-[2-[5-formyl-3-[4-(trifluoromethoxy)phenyl]indol-1-yl]ethyl]carbamate (07-2-3)

To a solution of compound 07-2-2 (1.28 g, 3.49 mmol, 1.0 eq) in dioxane (30 mL) was added [4-(trifluoromethoxy)phenyl]boronic acid (1.08 g, 5.24 mmol, 1.5 eq), K₂CO₃ (964 mg, 6.98 mmol, 2.0 eq) and Pd(PPh₃)₄(201 mg, 174.5 μmol, 0.05 eq). The mixture was stirred at 80° C. for 3 h under N₂, poured into H₂O (50 mL) and extracted with DCM (100 mL*3). The combined organic layers were dried over anhydrous Na₂SO₄, filtered, and concentrated under reduced pressure. The residue was purified by column chromatography (SiO₂) to give compound 07-2-3 (940 mg, 60%). M+H⁺=449.3 (LCMS).

### Step 4: tert-butyl N-[2-[5-[[4-[(2,6-dichlorophenyl)methyl]piperazin-1-yl]methyl]-3-[4-(trifluoromethoxy)phenyl]indol-1-yl]ethyl]carbamate (07-2-4)

To a solution of compound 07-2-3 (940 mg, 2.10 mmol, 1.0 eq) in DCE (10 mL) were added 1-[(2,6-dichlorophenyl)methyl]piperazine (566 mg, 2.31 mmol, 1.1 eq) and AcOH (126 mg, 2.10 mmol, 120.1 μL, 1.0 eq). After 30 min, NaBH(OAc)₃ (890 mg, 4.20 mmol, 2.0 eq) was added and the mixture was stirred at 20° C. for 11.5 h. The reaction mixture was diluted with aqueous NaHCO₃ (30 mL) and extracted with DCM (20 mL*3). The combined organic layers were washed with brines (20 mL*2), dried over anhydrous Na₂SO₄, filtered, and concentrated under reduced pressure. The residue was purified by column chromatography (SiO₂) to give compound 07-2-4 (970 mg, 68%). M+H⁺=677.3 (LCMS).

### Step 5: 2-[5-[[4-[(2,6-dichlorophenyl)methyl]piperazin-1-yl]methyl]-3-[4-(trifluoromethoxy)phenyl]indol-1-yl]ethanamine (07-2)

A solution of compound 07-2-4 (450 mg, 664.1 μmol, 1.0 eq) in HCl/EtOAc (3.0 mL) was stirred at 20° C. for 2 h. The reaction mixture was concentrated under reduced pressure to give a crude product, part of which (0.28 g) was purified by prep-HPLC (HCl condition) to give compound 07-2 (110 mg, 47%, 2 HCl). M+H⁺=577.2 (LCMS). ¹H NMR (MeOD, 400 MHz): δ 8.23 (s, 1H), 7.56 (d, J=8.8 Hz, 2H), 7.74 (t, J=8.4 Hz, 2H), 7.56 (d, J=7.2 Hz, 4H), 7.39 (d, J=8 Hz, 2H), 4.87-4.61 (m, 6H), 3.68 (br.s, 8H), 3.48 (m, J=6.4 Hz, 2H).

The following compounds are synthesized in similar procedures as described above for the preparation of 07-2.

| | | | | ¹H NMR |
|---|---|---|---|---|
| Comp | | | Mass | (MeOD, 400 |
| ID | Structure | Chemical Name | (M + H⁺) | MHz) |
| 07-12 | 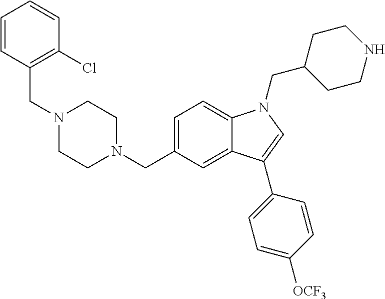 [SMILES: FC(F)(F)Oc1ccc(-c2cn(CC3CCNCC3)c3ccc(CN4CCN(Cc5ccccc5Cl)CC4)cc23)cc1] | 5-((4-(2- chlorobenzyl) piperazin- 1-yl)methyl)-1- (piperidin-4- ylmethyl)-3-(4- (trifluoromethoxy) phenyl)-1H-indole dihydrochloride | Calc'd for C₃₃H₃₇Cl F₃N₄O: 597.3; Found: 597.3 | δ 8.21 (d, J = 1.1 Hz, 1H), 7.89- 7.79 (m, 3H), 7.74-7.63 (m, 2H), 7.61-7.56 (m, 1H), 7.55- 7.44 (m, 3H), 7.36 (d, J = 1.9 Hz, 2H), 4.66 (d, J = 15.7 Hz, 4H), 4.26 (d, J = 7.3 Hz, 2H), 3.89-3.65 (m, 8H), 3.47-3.35 (m, 2H), 3.02- 2.90 (m, 2H), 2.31 (br s, 1H), 1.83 (br d, J = 12.8 Hz, 2H), 1.69-1.52 (m, 2H) |
| 07-38 | 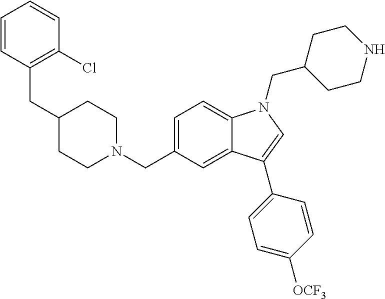 [SMILES: FC(F)(F)Oc1ccc(-c2cn(CC3CCNCC3)c3ccc(CN4CCC(Cc5ccccc5Cl)CC4)cc23)cc1] | 5-((4-(2- chlorobenzyl) piperidin-1- yl)methyl)-1- (piperidin-4- ylmethyl)-3-(4- (trifluoromethoxy) phenyl)-1H-indole dihydrochloride | Calc'd for C₃₄H₃₈Cl F₃N₃O: 596.3; Found: 596.3 | δ 8.07 (s, 1H), 7.80-7.79 (m, 1H), 7.78-7.76 (m, 1H), 7.69- 7.65 (m, 2H), 7.40 (br d, J = 1.32 Hz, 1H), 7.36 (dt, J = 5.07, 2.54 Hz, 3H), 7.27-7.25 (m, 1H), 7.22- 7.18 (m, 1H), 4.40 (s, 2H), 4.26 (d, J = 7.06 Hz, 2H), 3.48 (br d, J = 12.35 Hz, 2H), 3.40 (br d, J = 12.79 Hz, 2H), 3.01-2.93 (m, 4H), 2.75 (d, J = 7.06 Hz, 2H), 2.30 (br dd, J = 7.50, 3.75 Hz, 1H), 2.03-1.96 (m, 1H), 1.93- 1.80 (m, 5H), 1.58 (q, J = 11.61 Hz, 4H) |
| 07-10 | 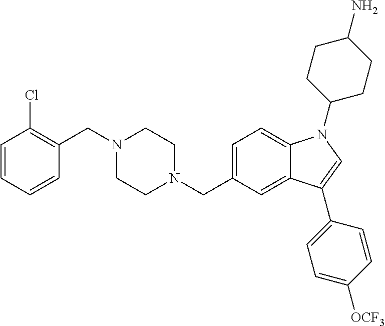 [SMILES: NC1CCC(n2cc(-c3ccc(OC(F)(F)F)cc3)c3cc(CN4CCN(Cc5ccccc5Cl)CC4)ccc32)CC1] | 4-(5-((4-(2- chlorobenzyl) piperazin-1-yl) methyl)-3-(4- (trifluoromethoxy) phenyl)-1H-indol- 1-yl)cyclohexan-1- amine dihydrochloride | Calc'd for C₃₃H₃₇Cl F₃N₄O: 597.3; Found: 597.3 | δ 8.21-8.14 (m, 1H), 7.91-7.77 (m, 4H), 7.71 (d, J = 8.6 Hz, 1H), 7.59-7.55 (m, 1H), 7.53-7.43 (m, 3H), 7.39- 7.32 (m, 2H), 4.68-4.49 (m, 5H), 3.86-3.57 (m, 8H), 2.32- 2.17 (m, 4H), 2.17-2.01 (m, 4H), 1.86-1.74 (m, 1H) |
| 07-11 | 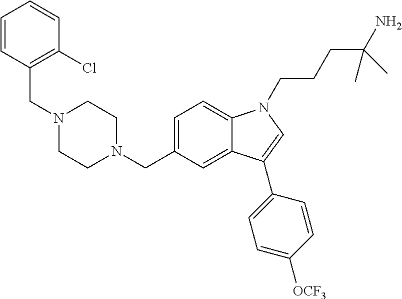 [SMILES: CC(C)(N)CCCn1cc(-c2ccc(OC(F)(F)F)cc2)c2cc(CN3CCN(Cc4ccccc4Cl)CC3)ccc21] | 5-(5-((4-(2- chlorobenzyl) piperazin-1-yl) methyl)-3-(4- (trifluoromethoxy) phenyl)-1H- indol-1-yl)-2- methylpentan-2- amine diformate | Calc'd for C₃₃H₃₉Cl F₃N₄O: 599.3; Found: 599.4 | δ 8.37 (brs, 2 H) 7.99 (s, 1 H) 7.72 (d, J = 8.8 Hz, 2 H) 7.63 (s, 1 H) 7.56 (d, J = 8.4 Hz,, 1 H) 7.46 (m, 1 H) 7.31-7.35 (m, 4 H) 7.25-7.27 (m, 2 H) 4.27-4.32 (m, 4H) 3.69 (s, 2 H) 3.13 (m, 4 H) 2.74 (m, 4 H) 1.93-1.99 (m, 2 H) 1.59-1.63 (m, 2 H) 1.26 (s, 6 H). |
| 06-67 | 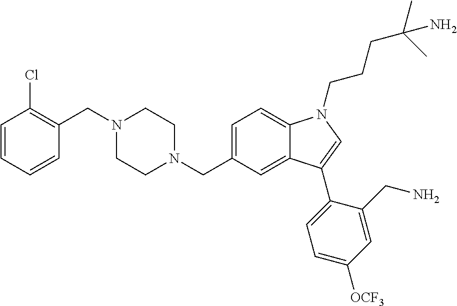 [SMILES: CC(C)(N)CCCn1cc(-c2ccc(OC(F)(F)F)cc2CN)c2cc(CN3CCN(Cc4ccccc4Cl)CC3)ccc21] | 5-(3-(2- (aminomethyl)-4- (trifluoromethoxy) phenyl)-5-((4-(2- chlorobenzyl) piperazin-1-yl) methyl)-1H- indol-1-yl)-2- methylpentan-2- amine dihydrochloride | Calc'd for C₃₂H₄₂Cl F₃N₅O: 628.3; Found: 628.4 | δ 7.77 (m, 1 H) 7.69 (s, 2 H) 7.62 (m, 1 H) 7.58-7.59 (m, 2 H) 7.54 (m, 1 H) 7.44-7 48 (m, 4 H) 4.59 (s 2 H) 4.55 (s 2 H) 4.39 (t, J = 6.4 Hz 2 H) 4.27 (s, 2 H) 3.63-3.72 (m, 8 H) 1.98-2.02 (m, 2 H) 1.67-1.70 (m, 2 H) 1.32 (s, 6 H) |

### Example 43: Synthesis of tert-butyl 4-[(5-formylindol-1-yl)methyl]piperidine-1-carboxylate (07-12-1)

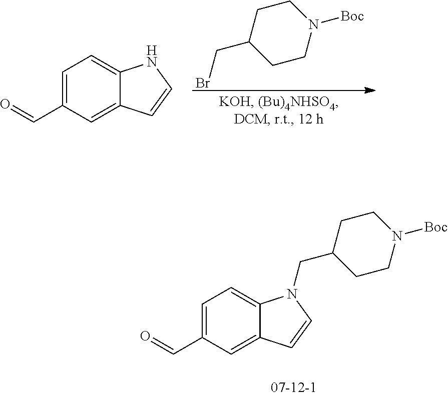

To a stirred solution of 1H-indole-5-carbaldehyde (800 mg, 5.51 mmol, 1.0 eq) and KOH (838 mg, 14.9 mmol, 2.7 eq) in DCM (10 mL) was added tetrabutylammonium hydrogen sulfate (1.87 g, 5.51 mmol, 1.0 eq) and tert-butyl 4-(bromomethyl)piperidine-1-carboxylate (1.99 g, 7.16 mmol, 1.3 eq) at 20° C. After 12 h, the reaction mixture was quenched by water (50 mL) and extracted with DCM (20 mL*3). The combined organic layers were washed with brine (30 mL), dried over anhydrous Na₂SO₄, filtered, and concentrated under reduced pressure. The residue was purified by column (SiO₂) to give compound 07-12-1 (370 mg, 1.08 mmol, 20% yield). ¹H NMR (CDCl₃, 400 MHz): δ 10.03 (s, 1H), 8.16 (d, J=1.1 Hz, 1H), 7.79 (dd, J=1.5, 8.6 Hz, 1H), 7.41 (d, J=8.6 Hz, 1H), 7.16 (d, J=3.3 Hz, 1H), 6.67 (dd, J=0.8, 3.2 Hz, 1H), 4.13 (q, J=7.1 Hz, 2H), 4.05 (d, J=7.3 Hz, 2H), 2.63 (br t, J=11.9 Hz, 2H), 2.04-1.95 (m, 1H), 1.61-1.51 (m, 2H), 1.45 (s, 9H), 1.26-1.15 (n, 2H).

### Example 44: Synthesis of tert-butyl N-[4-(5-formylindol-1-yl)cyclohexyl]carbamate (07-10-3)

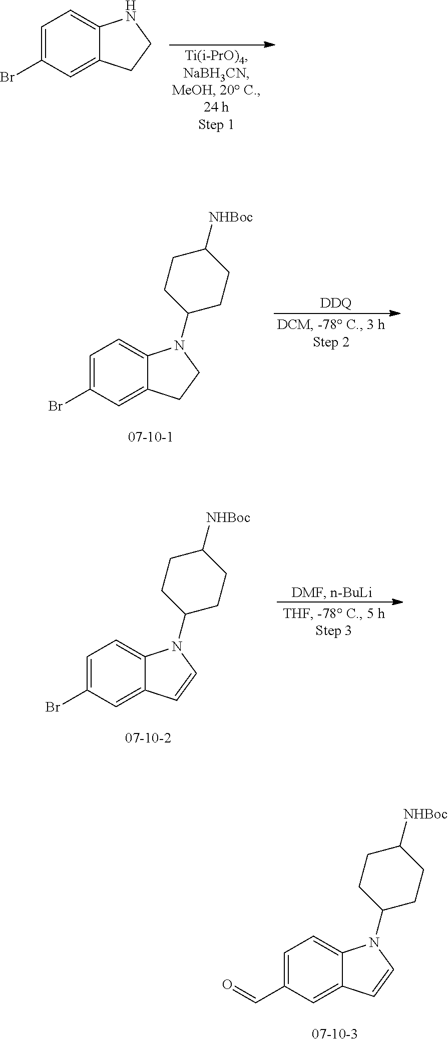

### Step 1: tert-butyl N-[4-(5-bromoindolin-1-yl)cyclohexyl]carbamate (07-10-1)

To a stirred solution of 5-bromoindoline (5.00 g, 25.2 mmol, 1.0 eq) and tert-butyl N-(4-oxocyclohexyl)carbamate (8.10 g, 37.9 mmol, 8.10 mL, 1.50 eq) in MeOH (100 mL) was added Ti(i-PrO)₄ (10.80 g, 38.0 mmol, 11.25 mL, 1.51 eq) at 20° C. After 12 h, NaBH₃CN (3.50 g, 55.7 mmol, 2.2 eq) was added to the mixture in portions. The resulting mixture was stirred at 20° C. for another 12 h, poured into water (300 mL), and filtered through a pad of Celite. The filter cake was washed with DCM (50 mL×10) and the organic layer was separated, washed with brine (200 mL), dried over anhydrous Na₂SO₄, filtered, and concentrated under reduced pressure. The residue was purified by column chromatography (SiO₂) to give compound 07-10-1 (5.00 g, 12.6 mmol, 50% yield). ¹H NMR (CDCl₃, 400 MHz): δ 7.07-6.99 (m, 2H), 6.20-6.11 (m, 1H), 3.47-2.98 (m, 4H), 2.90-2.77 (m, 2H), 2.03 (br d, J=12.3 Hz, 1H), 1.85 (br d, J=13.8 Hz, 1H), 1.81-1.72 (m, 1H), 1.62 (br d, J=12.7 Hz, 1H), 1.57-1.50 (m, 1H), 1.49-1.40 (m, 2H), 1.38 (d, J=5.6 Hz, 9H), 1.22-1.07 (m, 1H).

### Step 2: tert-butyl N-[4-(5-bromoindol-1-yl)cyclohexyl]carbamate (07-10-2)

To a solution of compound 07-10-1 (4.10 g, 10.4 mmol, 1.0 eq) in DCM (50 mL) was added DDQ (2.35 g, 10.4 mmol, 1.0 eq) at −78° C. The mixture was stirred at −78° C. for 5 hour, poured into H₂O (150 mL), filtered, and the filter residue was washed with DCM (30 mL*3). The filtrate was extracted with DCM (50 mL*3). The combined organic layers were washed with brine (80 mL), dried over anhydrous Na₂SO₄, filtered, and concentrated under reduced pressure. The residue was purified by column chromatography (SiO₂) to give compound 07-10-2 (2.70 g, 6.80 mmol, 66% yield). M+H⁺=393.2 (LCMS). ¹H NMR (CDCl₃, 400 MHz): δ 7.81-7.77 (m, 1H), 7.34-7.27 (m, 2H), 7.26-7.22 (m, 1H), 6.51-6.48 (m, 1H), 4.27-4.17 (m, 1H), 3.61 (br s, 1H), 2.30-2.17 (m, 3H), 2.06 (br d, J=12.0 Hz, 2H), 1.97-1.82 (m, 3H), 1.53 (d, J=5.5 Hz, 9H).

### Step 3: tert-butyl N-[4-(5-formylindol-1-yl)cyclohexyl]carbamate (07-10-3)

To a stirred solution of compound 07-10-2 (2.50 g, 6.36 mmol, 1.0 eq) in THF (40 mL) was added n-BuLi (2.5 M, 5.19 mL, 2.04 eq) dropwise at −78° C. under N₂. After 1 h at −78° C., a solution of DMF (520 mg, 7.12 mmol, 548 μL, 1.12 eq) in THF (5 mL) was added to the mixture dropwise. The resulting mixture was stirred at −78° C. for 4 h under N₂. The mixture was quenched with water (50 mL) and extracted with EtOAc (20 mL*3). The combined organic layers were washed with brine (30 mL), dried over anhydrous Na₂SO₄, filtered, and concentrated under reduced pressure. The residue was purified by column chromatography (SiO₂) to give compound 07-10-3 (1.80 g, 5.18 mmol, 81% yield). M+H⁺=343.2 (LCMS). ¹H NMR (CDCl₃, 400 MHz): δ 9.96-9.93 (m, 1H), 8.10-8.04 (m, 1H), 7.73-7.67 (m, 1H), 7.40-7.33 (m, 1H), 7.29 (d, J=3.4 Hz, 0.31H), 7.21 (d, J=3.4 Hz, 0.66H), 6.63-6.58 (m, 1H), 4.76 (br d, J=6.1 Hz, 0.29H), 4.44 (br s, 0.57H), 4.30-4.13 (m, 1H), 3.87 (br s, 0.3H), 3.51 (br s, 0.69H), 2.21-2.06 (m, 3H), 2.00-1.93 (m, 1H), 1.91-1.68 (m, 3H), 1.40 (d, J=5.8 Hz, 9H).

### Example 45: Synthesis of 1-(4-methyl-4-nitro-pentyl)indole-5-carbaldehyde (07-11-4)

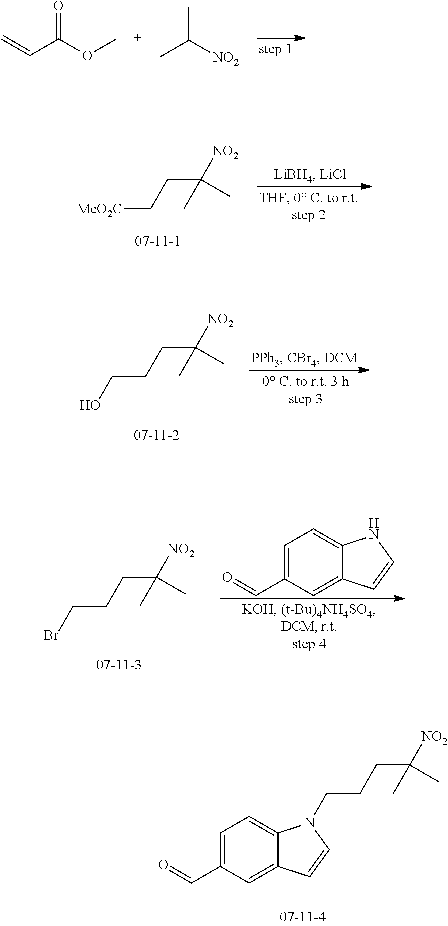

### Step 1: methyl 4-methyl-4-nitro-pentanoate (07-11-1)

To a solution of methyl acrylate (6.80 g, 79.0 mmol, 7.09 mL, 1.1 eq) and 2-nitropropane (6.40 g, 71.8 mmol, 6.46 mL, 1.0 eq) in dioxane (100 mL) was added benzyl(trimethyl)ammonium hydroxide (30.0 g, 71.8 mmol, 32.6 mL, 1.0 eq) drop-wise at 20° C. The mixture was stirred at 85° C. for 24 h, cooled to room temperature and concentrated. The residue was dissolved in MTBE (200 mL), stirred for 0.5 h, and filtered. The filtrate was concentrated to give a residue which was purified by column chromatography (SiO₂) to give compound 07-11-1 (6.20 g, 35.4 mmol, 49% yield).

### Step 2: 4-methyl-4-nitro-pentan-1-ol (07-11-2)

To a solution of compound 07-11-1 (6.10 g, 34.8 mmol, 1.0 eq) in THF (60 mL) was added LiBH₄ (1.14 g, 52.2 mmol, 1.5 eq) in one portion at 0° C. After addition, the mixture was stirred at 20° C. for 18 h, quenched with H₂O (10 mL) at 0° C. and stirred for 10 min. The mixture was extracted with EtOAc (20 mL*2) and the combined organic layers were washed with H₂O (20 mL), dried over anhydrous Na₂SO₄, and filtered. The filtrate was concentrated under reduced pressure. The residue was purified by column chromatography (SiO₂) to give compound 07-11-2 (4.50 g, 30.6 mmol, 88% yield). ¹H NMR (CDCl₃, 400 MHz): δ 3.56-3.59 (m, 2H) 1.90-1.94 (m, 2H) 1.53 (s, 6H) 1.43-1.48 (m, 2H).

### Step 3: 1-bromo-4-methyl-4-nitro-pentane (07-11-3)

To a solution of 4-methyl-4-nitro-pentan-1-ol (3.80 g, 25.8 mmol, 1.0 eq) in DCM (40 mL) was added carbon tetrabromide (12.8 g, 38.7 mmol, 1.5 eq) and PPh₃ (10.2 g, 38.7 mmol, 1.5 eq) at 0° C. The resulting mixture was stirred at 20° C. for 3 h, filtered, and the filtrate was concentrated under reduced pressure to give a residue which was purified by column chromatography (SiO₂) to give compound 07-11-3 (4.70 g, 22.4 mmol, 87% yield). ¹H NMR (CDCl₃, 400 MHz): δ 3.31-3.34 (m, 2H) 1.97-2.01 (m, 2H) 1.75-1.79 (m, 2H) 1.53 (s, 6H).

### Step 4: 1-(4-methyl-4-nitro-pentyl)indole-5-carbaldehyde (07-11-4)

To a solution of 1H-indole-5-carbaldehyde (3.25 g, 22.4 mmol, 1.0 eq) in DCM (45 mL) was added 07-11-3 (4.70 g, 22.4 mmol, 1.0 eq), tetrabutylammonium hydrogen sulfate (7.60 g, 22.4 mmol, 1.0 eq) and KOH (3.77 g, 67.1 mmol, 3.0 eq). The mixture was stirred at 20° C. for 18 h, poured into H₂O (45 mL), and extracted with DCM (50 mL*2). The combined organic layers were washed with H₂O (50 mL), brine (50 mL), dried over anhydrous Na₂SO₄, and filtered. The filtrate was concentrated under reduced pressure to give a residue. The residue was purified by column chromatography (SiO₂) to give compound 07-11-4 (4.10 g, 14.7 mmol, 66% yield). ¹H NMR (CDCl₃, 400 MHz): δ 9.95 (brs, 1H) 8.08 (s, 1H) 7.70-7.73 (m, 1H) 7.30 (d, J=8.8 Hz 1H) 7.09 (d, J=3.2 Hz 1H) 6.60 (d, J=2.8 Hz 1H) 4.04-4.12 (m, 2H) 1.74-1.83 (m, 4H) 1.46 (s, 6H).

### Example 46: Synthesis of 4-[5-[[4-[(2,6-dichlorophenyl)methyl]piperazin-1-yl]methyl]-3-[4-(trifluoromethoxy)phenyl]indol-1-yl]butan-1-amine (07-4)

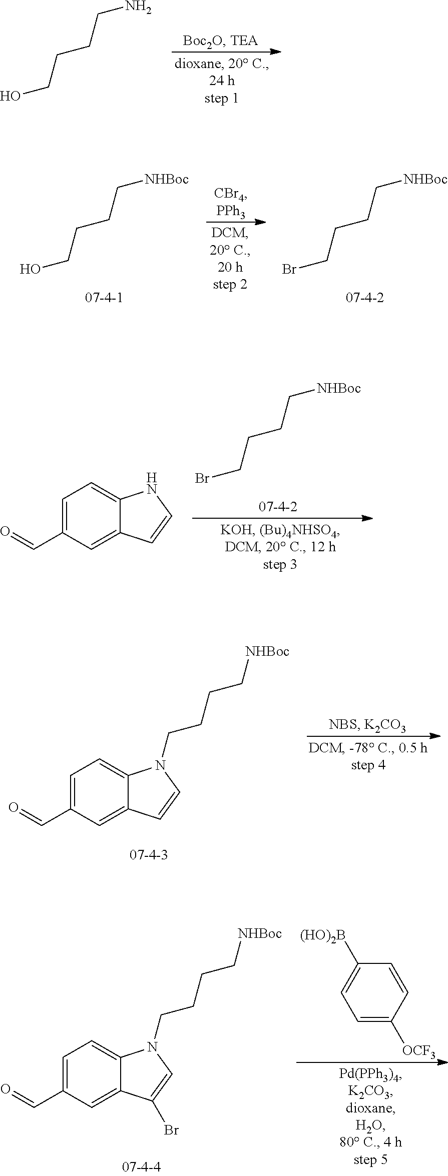

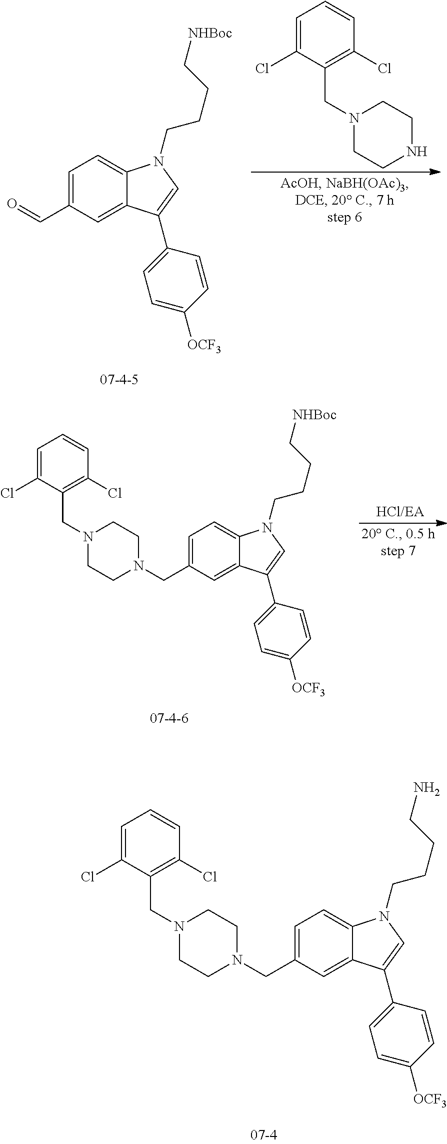 [SMILES: CC(C)(C)OC(=O)NCCCCn1cc(-c2ccc(OC(F)(F)F)cc2)c2cc(C=O)ccc21 ; Clc1cccc(Cl)c1CN1CCNCC1 ; CC(C)(C)OC(=O)NCCCCn1cc(-c2ccc(OC(F)(F)F)cc2)c2cc(CN3CCN(Cc4c(Cl)cccc4Cl)CC3)ccc21 ; NCCCCn1cc(-c2ccc(OC(F)(F)F)cc2)c2cc(CN3CCN(Cc4c(Cl)cccc4Cl)CC3)ccc21]

### Step 1: tert-butyl N-(4-hydroxybutyl)carbamate (07-4-1)

A mixture of 4-aminobutan-1-ol (10.0 g, 112.1 mmol, 10.4 mL, 1.0 eq), tert-butoxycarbonyl tert-butyl carbonate (25.7 g, 117.8 mmol, 27.1 mL, 1.05 eq) and DIEA (21.7 g, 168.2 mmol, 29.3 mL, 1.5 eq) in DCE (400 mL) was stirred at 20° C. for 24 h, diluted with water (400 mL) and extracted with DCM (100 mL*3). The combined organic layers were washed with brine (100 mL), dried over anhydrous Na₂SO₄, filtered and concentrated under reduced pressure. The residue was purified by column chromatography (SiO₂) to give compound 07-4-1 (12.0 g, 60.2 mmol, 54% yield). ¹H NMR (CDCl₃, 400 MHz): δ 3.67 (d, J=4.85 Hz, 2H), 3.16 (d, J=5.29 Hz, 2H), 1.54-1.62 (m, 4H), 1.44 (s, 9H).

### Step 2: tert-butyl N-(4-bromobutyl)carbamate (07-4-2)

A solution of compound 07-4-1 (11.0 g, 58.1 mmol, 1.0 eq) in DCM (200 mL) were added CBr₄ (39.5 g, 119.1 mmol, 2.05 eq) and PPh₃ (32.9 g, 125.6 mmol, 2.16 eq) at 20° C., and the resulting mixture was stirred at 20° C. for 20 h, diluted with water (100 mL) and extracted with DCM (100 mL*3), and the mixture was filtered and concentrated under reduced pressure to give a residue which was purified by column chromatography (SiO₂) to give compound 07-4-2 (12.0 g, 45.2 mmol, 78% yield). ¹H NMR (CDCl₃, 400 MHz): δ 3.41 (t, J=6.62 Hz, 2H), 3.14 (d, J=6.17 Hz, 2H), 1.83-1.94 (m, 2H), 1.62 (quin, J=7.28 Hz, 2H), 1.36-1.49 (m, 9H).

Step 3 to step 7 are carried out according to similar procedures as described in step 1 to step 5 of the synthesis of 07-2.

### tert-butyl N-[4-(5-formylindol-1-yl)butyl]carbamate (07-4-3)

¹H NMR (CDCl₃, 400 MHz): δ 10.02 (s, 1H), 8.15 (s, 1H), 7.78 (d, J=8.82 Hz, 1H), 7.42 (d, J=8.38 Hz, 1H), 7.19 (d, J=3.09 Hz, 1H), 6.65 (d, J=3.09 Hz, 1H), 4.20 (t, J=7.06 Hz, 2H), 3.15 (d, J=6.17 Hz, 2H), 1.83-1.94 (m, 2H), 1.47-1.53 (m, 2H), 1.43 (s, 9H).

### tert-butyl N-[4-(3-bromo-5-formyl-7,7a-dihydroindol-1-yl)butyl]carbamate (07-4-4)

M+H⁺=397.2 (LCMS). ¹H NMR (CDCl₃, 400 MHz): δ 10.09 (s, 1H), 8.11 (s, 1H), 7.85 (d, J=8.53 Hz, 1H), 7.46 (d, J=9.03 Hz, 1H), 7.29 (s, 1H), 4.16-4.26 (m, 2H), 3.19 (d, J=6.02 Hz, 2H), 1.90 (quin, J=7.40 Hz, 2H), 1.49-1.57 (m, 2H), 1.46 (s, 9H).

### tert-butyl N-[4-[5-formyl-3-[4-(trifluoromethoxy)phenyl]indol-1-yl]butyl]carbamate (07-4-5)

M+H⁺=421.2 (LCMS).

### tert-butyl N-[4-[5-[[4-[(2,6-dichlorophenyl)methyl]piperazin-1-yl]methyl]-3-[4-(trifluoromethoxy)phenyl]indol-1-yl]butyl]carbamate (07-4-6)

M+H⁺=705.2 (LCMS).

### 4-[5-[[4-[(2,6-dichlorophenyl)methyl]piperazin-1-yl]methyl]-3-[4-(trifluoromethoxy)phenyl]indol-1-yl]butan-1-amine (07-4)

M+H⁺=605.3 (LCMS). ¹H NMR (MeOD, 400 MHz): δ 8.18 (s, 1H), 7.82 (d, J=8.38 Hz, 2H), 7.70 (s, 1H), 7.65 (d, J=8.38 Hz, 1H), 7.53-7.57 (m, 1H), 7.44-7.50 (m, 1H), 7.34 (d, J=8.38 Hz, 1H), 4.62 (d, J=17.64 Hz, 4H), 4.35 (t, J=6.40 Hz, 2H), 3.73 (br. s., 8H), 2.93 (t, J=7.50 Hz, 2H), 1.93-2.05 (m, 2H), 1.62-1.73 (m, 2H).

### Example 47: Synthesis of 4-[5-[[8-[(2-chlorophenyl)methyl]-3,8-diazabicyclo[3.2.1]octan-3-yl]methyl]-3-(4-methoxyphenyl)indol-1-yl]butan-1-amine (07-16)

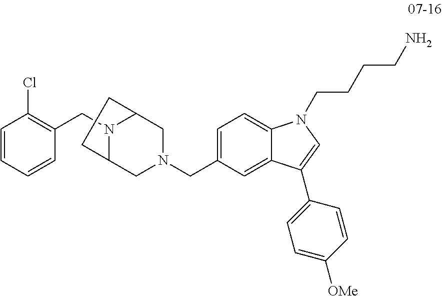 [SMILES: COc1ccc(-c2cn(CCCCN)c3ccc(CN4CC5CCC(C4)N5Cc4ccccc4Cl)cc23)cc1]

Compound 07-16 was synthesized according to a procedure similar to the procedure described for the preparation of 07-4. M+H⁺=543.4 (LCMS). ¹H NMR (MeOD, 400 MHz): δ ppm 8.04 (s, 1H), 7.83 (br d, J=6.39 Hz, 1H), 7.36-7.59 (m, 8H), 6.95-7.01 (m, 1H), 6.94-6.96 (m, 1H), 4.41 (br s, 4H), 4.30 (t, J=6.84 Hz, 2H), 4.18 (br s, 2H), 3.56-3.89 (m, 5H), 3.37-3.54 (m, 2H), 2.89 (t, J=7.72 Hz, 2H), 2.57 (br s, 2H), 2.40 (br d, J=8.82 Hz, 2H), 1.90-1.99 (m, 2H), 1.59-1.68 (m, 2H).

### Example 48: Synthesis of (1S)-3-[(1S)-5-[[4-[(2,6-dichlorophenyl)methyl]piperazin-1-yl]methyl]-3-[4-(trifluoromethoxy)phenyl]indol-1-yl]cyclopentanamine (07-1)

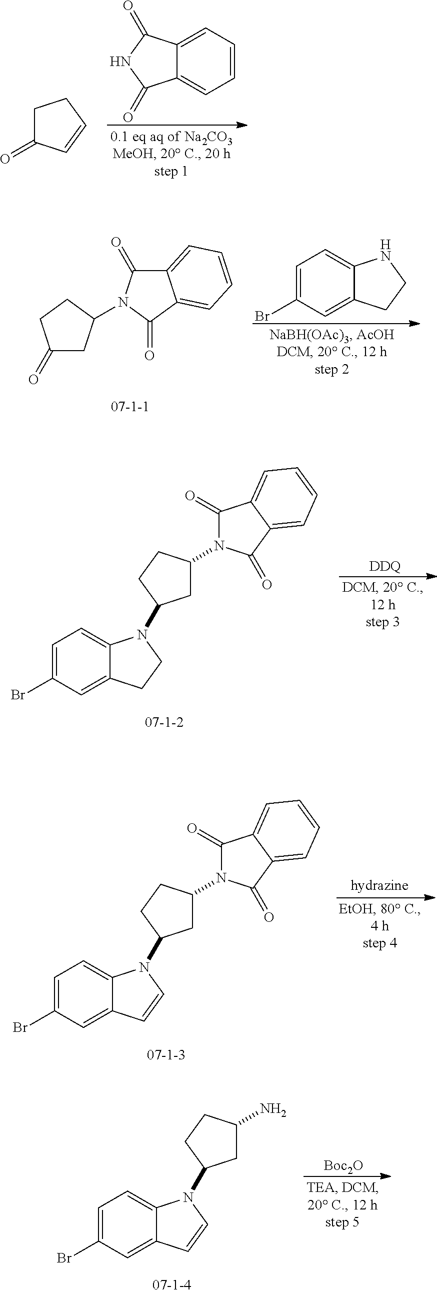

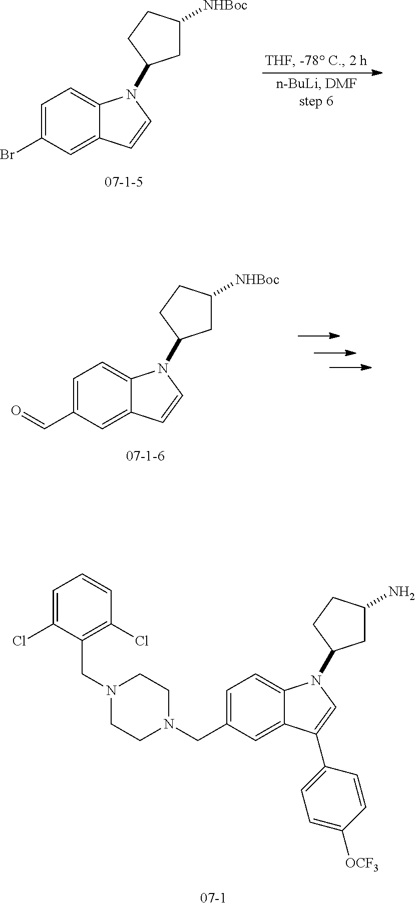

### Step 1: 2-(3-oxocyclopentyl)isoindoline-1,3-dione (07-1-1)

To a mixture of cyclopent-2-en-1-one (10.0 g, 121.8 mmol, 10.2 mL, 1.0 eq) and isoindoline-1,3-dione (17.9 g, 121.8 mmol, 1.0 eq) in MeOH (90 mL) was added Na₂CO₃ (2 M, 7.92 mL, 0.13 eq) dropwise. The solution was stirred at 20° C. for 20 h and the precipitated solid was collected, washed with MeOH (50 ml) and dried to give the crude product. The crude product was further washed with DCM (200 mL) and dried to give compound 07-1-1 (13.5 g, 53.0 mmol, 44% yield).

### Step 2: 2-[3-(5-bromoindolin-1-yl)cyclopentyl]isoindoline-1,3-dione (07-1-2)

To a solution of 5-bromoindoline (1.00 g, 5.05 mmol, 1.0 eq) in DCM (20 mL) was added AcOH (303 mg, 5.05 mmol, 289.0 μL, 1.0 eq). After 1 h, NaBH(OAc)₃ (1.28 g, 6.06 mmol, 1.2 eq) was added and the resulting mixture was stirred at 20° C. for 11 h. The reaction mixture was washed with saturated NaHCO₃ solution (20 mL*3) and the combined aqueous layers were extracted with DCM (30 mL*3). The combined organic layers were washed with brines (20 mL*2), dried over anhydrous Na₂SO₄, filtered, and concentrated under reduced pressure to give compound 07-1-2 (2.20 g, 95% yield). M+H⁺=412.1 (LCMS).

### Step 3: 2-[3-(5-bromoindol-1-yl)cyclopentyl]isoindoline-1,3-dione (07-1-3)

To a solution of compound 07-1-2 (2.20 g, 1.0 eq) in DCM (30 mL) was added DDQ (1.46 g, 6.42 mmol, 1.2 eq), and the mixture was stirred at 20° C. for 12 h. The reaction mixture was washed with saturated NaHCO₃ solution (30 mL*2) and the combined aqueous layers were extracted with DCM (50 mL*3). The combined organic layers were washed with brines (50 mL*2), dried over anhydrous Na₂SO₄, filtered and concentrated under reduced pressure. The residue was purified by column chromatography (SiO₂) to give compound 07-1-3 (1.10 g, 2.69 mmol, 50% yield). M+H⁺=409.1 (LCMS).

### Step 4: 3-(5-bromoindol-1-yl)cyclopentanamine (07-1-4)

To a solution of compound 07-1-3 (1.20 g, 2.93 mmol, 1.0 eq) in EtOH (100 mL) was added hydrazine (1.88 g, 58.6 mmol, 2.11 mL, 20.0 eq) and the resulting mixture was heated at 80° C. for 4 h. The solution was filtered and the filtrate was concentrated. The residue was diluted with water (50 ml), and extracted with EtOAc (100 ml). The organic phase was separated, dried over anhydrous Na₂SO₄, filtered and concentrated to give compound 07-1-4 (600 mg, 2.15 mmol, 73% yield). M+H⁺=281.1 (LCMS).

### Step 5: tert-butyl N-[(1S)-3-[(1S)-5-bromoindol-1-yl]cyclopentyl]carbamate (07-1-5)

To a solution of compound 07-1-4 (600 mg, 2.15 mmol, 1.0 eq) and tert-butoxycarbonyl tert-butyl carbonate (938 mg, 4.30 mmol, 987.5 μL, 2.0 eq) in DCM (7 mL) was added TEA (652 mg, 6.45 mmol, 893.7 μL, 3.0 eq) dropwise. The solution was stirred at 20° C. for 12 h under N₂ and concentrated under reduced pressure to give a residue. The residue was purified by column chromatography (SiO₂) to give compound 07-1-5 (740 mg, 1.91 mmol, 89% yield). M+H⁺=379.1 (LCMS). ¹H NMR (CDCl₃, 400 MHz): δ 7.74 (d, J=1.32 Hz, 1H), 7.31-7.17 (m, 3H), 6.49-6.42 (m, 1H), 4.80-4.60 (m, 2H), 2.69 (dt, J=13.56, 7.11 Hz, 1H), 2.35-2.17 (m, 2H), 2.13-2.01 (m, 1H), 1.85-1.67 (m, 2H), 1.47 (s, 9H).

### Step 6: tert-butyl N-[(1S)-3-[(1S)-5-formylindol-1-yl]cyclopentyl]carbamate (07-1-6)

To a mixture of compound 07-1-5 (490 mg, 1.29 mmol, 1.0 eq) in THF (12 mL) at −78° C. was added n-BuLi (2.5 M, 1.03 mL, 2.0 eq). After 30 min, DMF (94 mg, 1.29 mmol, 99.4 μL, 1.0 eq) was added drop-wise and the resulting mixture was stirred at −78° C. for another 1 h. The reaction mixture was poured into H₂O (80 mL) and extracted with EtOAc (30 mL*3). The combined organic layers were washed with brine (50 mL), dried over anhydrous Na₂SO₄, filtered and the filtrate was concentrated under reduced pressure. The residue was purified by column chromatography (SiO₂) to give compound 07-1-6 (160 mg, 487.2 μmol, 38% yield). M+H⁺=329.2 (LCMS). ¹H NMR (CDCl₃, 400 MHz): δ 10.03 (s, 1H), 8.15 (s, 1H), 7.78 (d, J=8.38 Hz, 1H), 7.45 (d, J=8.82 Hz, 1H), 7.33 (d, J=3.53 Hz, 1H), 7.31-7.26 (m, 1H), 6.73-6.64 (m, 1H), 4.90-4.79 (m, 1H), 4.68 (br. s., 1H), 2.74 (dt, J=13.56, 7.11 Hz, 1H), 2.43-2.28 (m, 2H), 2.22 (dd, J=14.55, 7.50 Hz, 1H), 1.90-1.77 (m, 2H), 1.47 (d, J=1.76 Hz, 9H).

### Step 7: (1S)-3-[(1S)-5-[[4-[(2,6-dichlorophenyl)methyl]piperazin-1-yl]methyl]-3-[4-(trifluoromethoxy)phenyl]indol-1-yl]cyclopentanamine (07-1)

The title compound was prepared from 07-1-6 according to similar procedures as described in the synthesis of 07-2. M+H⁺=617.3. ¹H NMR (MeOD, 400 MHz): δ 8.19 (s, 1H), 7.89-7.83 (m, 3H), 7.71 (d, J=8.38 Hz, 1H), 7.58-7.54 (m, 2H), 7.52-7.46 (m, 2H), 7.36 (d, J=8.38 Hz, 2H), 4.69 (s, 2H), 4.62 (s, 2H), 3.86-3.61 (m, 9H), 2.86-2.77 (m, 1H), 2.54-2.44 (m, 1H), 2.43-2.27 (m, 3H), 2.13-2.00 (m, 2H).

### Example 49: Synthesis of [3-[5-[[4-[(2-chlorophenyl)methyl]piperazin-1-yl]methyl]-3-[4-(trifluoromethoxy)phenyl]indol-1-yl]phenyl]methanamine (07-13)

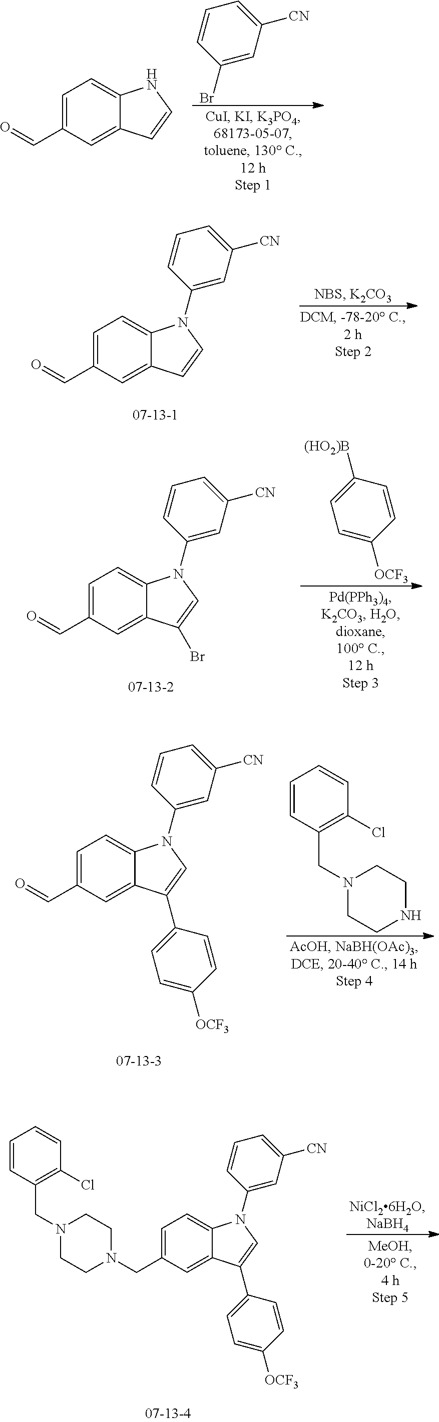

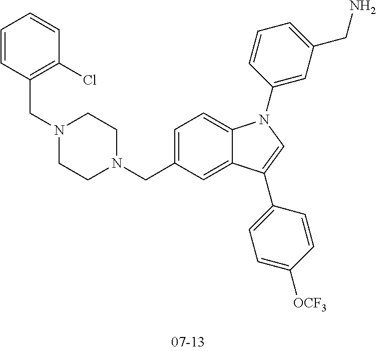 [SMILES: NCc1cccc(-n2cc(-c3ccc(OC(F)(F)F)cc3)c3cc(CN4CCN(Cc5ccccc5Cl)CC4)ccc32)c1]

### Step 1: 3-(5-formylindol-1-yl)benzonitrile (07-13-1)

To a solution of 1H-indole-5-carbaldehyde (1.00 g, 6.89 mmol, 1.0 eq) and 3-bromobenzonitrile (1.88 g, 10.3 mmol, 1.5 eq) in toluene (30 mL) were added (1S,2S)—N1,N2-dimethylcyclohexane-1,2-diamine (294 mg, 2.07 mmol, 0.3 eq), CuI (131 mg, 689 μmol, 0.1 eq), K₃PO₄ (4.39 g, 20.6 mmol, 3.0 eq) and KI (1.72 g, 10.3 mmol, 1.5 eq). The mixture was stirred at 130° C. for 12 h, poured into H₂O (100 mL) and extracted with EtOAc (30 mL*3). The combined organic layers were washed with brine (50 mL), dried over anhydrous Na₂SO₄, filtered and concentrated under reduced pressure to give a residue. The residue was washed with petroleum ether:EtOAc=5:1 (15 mL*3) and dried in vacuum to give compound 07-13-1 (1.10 g, 54% yield). ¹H NMR (CDCl₃, 400 MHz): δ 10.11-10.06 (m, 1H), 8.24 (s, 1H), 7.87-7.77 (m, 3H), 7.75-7.69 (m, 2H), 7.58 (d, J=8.7 Hz, 1H), 7.42 (d, J=3.4 Hz, 1H), 6.90 (d, J=3.3 Hz, 1H)

### Step 2: 3-(3-bromo-5-formyl-indol-1-yl)benzonitrile (07-13-2)

To a solution of 07-13-1 (1.10 g, 1.0 eq) and K₂CO₃ (932 mg, 6.74 mmol, 1.51 eq) in DCM (20 mL) was added NBS (795 mg, 4.47 mmol, 1.0 eq) at −78° C. in portions. After 1 h at −78° C., the mixture was allowed to warm to 20° C. and was stirred for 1 h. The reaction was quenched with water (40 mL) and extracted with DCM (20 mL*3). The combined organic layers were washed with brine (30 mL), dried over anhydrous Na₂SO₄, filtered, and the filtrate was concentrated under reduced pressure to give compound 07-13-2 (1.50 g, crude) which was used directly in next step without further purification. ¹H NMR (CDCl₃, 400 MHz): δ 10.13 (s, 1H), 8.20 (s, 1H), 7.90 (br d, J=8.4 Hz, 1H), 7.83-7.80 (m, 1H), 7.78-7.71 (m, 3H), 7.57 (br d, J=8.4 Hz, 1H), 7.48 (s, 1H).

### Step 3: 3-[5-formyl-3-[4-(trifluoromethoxy)phenyl]indol-1-yl]benzonitrile (07-13-3)

A mixture of compound 07-13-2 (1.20 g, 3.69 mmol, 1.0 eq), [4-(trifluoromethoxy)phenyl] boronic acid (1.10 g, 5.35 mmol, 1.45 eq), Pd(PPh3)4 (213 mg, 184.5 μmol, 0.05 eq) and K₂CO₃ (1000 mg, 7.23 mmol, 1.96 eq) in dioxane (20 mL) and H₂O (2 mL) was degassed and heated to 100° C. for 12 h under N₂. After the reaction mixture was cooled to room temperature, it was diluted with water (50 mL) and extracted with DCM (25 mL*4). The combined organic layers were washed with brine (50 mL), dried over anhydrous Na₂SO₄, filtered and the filtrate was concentrated. The residue was purified by column chromatography (SiO₂) to give compound 07-13-3 (800 mg, 26% yield). M+H⁺=407.1 (LCMS).

### Step 4: 3-[5-[[4-[(2-chlorophenyl)methyl]piperazin-1-yl]methyl]-3-[4-(trifluoromethoxy)phenyl]indol-1-yl]benzonitrile (07-13-4)

To a solution of 07-13-3 (800 mg, 1.97 mmol, 1.0 eq) and 1-[(2-chlorophenyl)methyl]piperazine (452 mg, 2.15 mmol, 1.09 eq) in DCE (10 mL) was added AcOH (118 mg, 1.97 mmol, 112.6 μL, 1.0 eq) at 20° C. Then the mixture was stirred at 40° C. for 2 h. After cooled to 20° C., NaBH(OAc)₃ (1.04 g, 4.90 mmol, 2.49 eq) was added in portions. The resulting mixture was stirred at 20° C. for 12 h. The reaction mixture was washed with saturated NaHCO₃ solution (50 mL*2) and the combined aqueous layers were extracted with DCM (50 mL*2). The combined organic layer was washed with brine (100 mL), dried over anhydrous Na₂SO₄, filtered, and the filtrate was concentrated under reduced pressure. The residue was purified by column chromatography (SiO₂) to give compound 07-13-4 (500 mg, crude).

### Step 5: [3-[5-[[4-[(2-chlorophenyl)methyl]piperazin-1-yl]methyl]-3-[4-(trifluoromethoxy)phenyl]indol-1-yl]phenyl]methanamine (07-13)

To a stirred solution of 07-13-4 (500 mg, 831.9 μmol, 1.0 eq) in MeOH (10 mL) was added NiCl₂*6H₂O (200 mg, 841.4 μmol, 1.0 eq), then the mixture was cooled to 0° C. NaBH₄ (157 mg, 4.15 mmol, 5.0 eq) was added in portions. The resulting mixture was allowed to warm to 20° C. and was stirred for 4 h. The reaction was quenched with water (30 mL), filtered through a pad of Celite and filter cake was washed with DCM (10 mL*5). The organic layer was separated and washed with brine (30 mL), filtered and the filtrate was concentrated under reduced pressure. The residue was purified by acidic prep-HPLC to give compound 07-13 (60 mg, 85.4 μmol, 10% yield, FA). M+H⁺=605.3 (LCMS). ¹H NMR (MeOD, 400 MHz): δ 8.49 (br s, 2H), 7.96 (s, 1H), 7.85-7.78 (m, 3H), 7.72 (s, 1H), 7.70-7.63 (m, 3H), 7.50 (br d, J=4.4 Hz, 1H), 7.46 (dd, J=2.2, 7.1 Hz, 1H), 7.40-7.34 (m, 3H), 7.31 (br d, J=8.4 Hz, 1H), 7.29-7.20 (m, 2H), 4.22 (s, 2H), 3.90 (s, 2H), 3.67 (s, 2H), 2.76 (br d, J=11.9 Hz, 4H), 2.64 (br s, 4H).

The following compounds are synthesized in similar procedures as described above for the preparation of 07-13.

| | | | | 1H NMR |
|---|---|---|---|---|
| | | | Mass | (MeOD, 400 |
| Comp ID | Structure | Chemical Name | (M + H⁺) | MHz) |
| 07-14 | 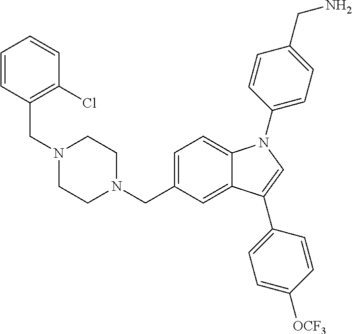 [SMILES: NCc1ccc(-n2cc(-c3ccc(OC(F)(F)F)cc3)c3cc(CN4CCN(Cc5ccccc5Cl)CC4)ccc32)cc1] | (4-(5-((4-(2- chlorobenzyl) piperazin-1- yl)methyl)-3-(4- (trifluoromethoxy) phenyl)-1H- indol-1- yl)phenyl) methanamine diformate | Calc'd for C₃₄H₃₃Cl F₃N₄O: 605.2; Found: 605.3 | δ 8.50 (s, 2H), 8.04 (s, 1H), 7.88-7.81 (m, 3H), 7.75-7.67 (m, 4H), 7.64 (d, J = 8.4 Hz, 1H), 7.47 (dd, J = 2.2, 7.1 Hz, 1H), 7.42-7.33 (m, 4H), 7.31- 7.23 (m, 2H), 4.23 (s, 2H), 4.13 (s, 2H), 3.72 (s, 2H), 2.99 (br s, 4H), 2.80-2.63 (m, 4H) |
| 07-23 | 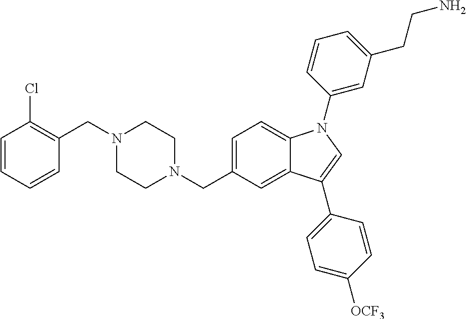 [SMILES: NCCc1cccc(-n2cc(-c3ccc(OC(F)(F)F)cc3)c3cc(CN4CCN(Cc5ccccc5Cl)CC4)ccc32)c1] | 2-(3-(5-((4-(2- chlorobenzyl) piperazin-1- yl)methyl)-3-(4- (trifluoromethoxy) phenyl)-1H- indol-1- yl)phenyl)ethan- 1-amine formate | Calc'd for C₃₅H₃₅Cl F₃N₄O: 619.2; Found: 619.3 | δ 8.54 (br s, 1H), 7.95 (s, 1H), 7.85- 7.83 (m, 1H), 7.82-7.81 (m 1H), 7.78 (s, 1H), 7.59 (dd, J = 4.3, 8.0 Hz, 2H), 7.56- 7.54 (m, 2H), 7.47 (dd, J = 2.0, 7.3 Hz, 1H), 7.39- 7.35 (m, 4H), 7.32-7.26 (m, 2H), 7.26- 7.23 (m, 1H), 3.84 (s, 2H), 3.68 (s, 2H), 3.26 (d, J = 8.2 Hz, 2H), 3.11- 3.06 (m, 2H), 2.77-2.56 (m, 8H) |

### Example 50: Synthesis of 5-[[4-[(2-chlorophenyl)methyl]-1-piperidyl]methyl]-1-(4-piperidyl)-3-[4-(trifluoromethoxy)phenyl]indole (07-37)

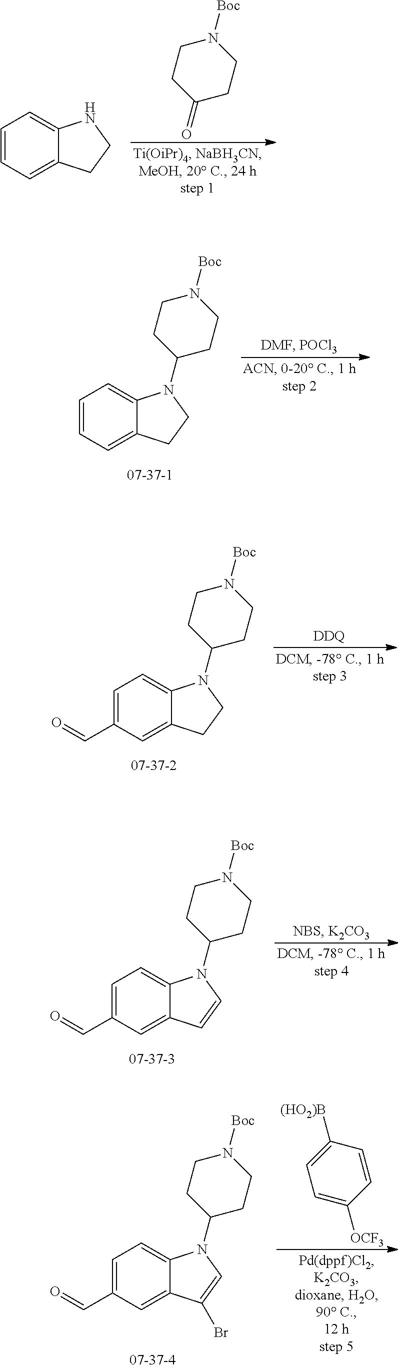

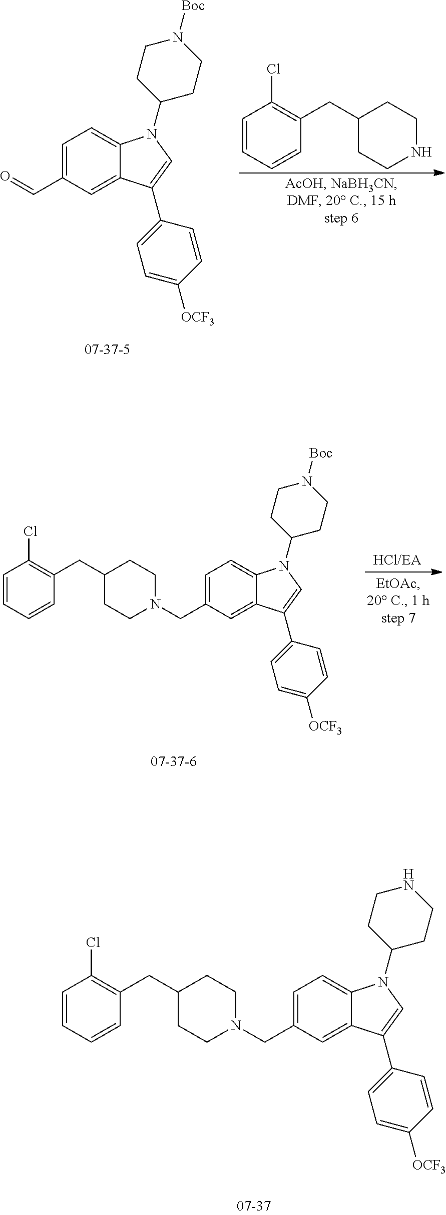 [SMILES: CC(C)(C)OC(=O)N1CCC(n2cc(-c3ccc(OC(F)(F)F)cc3)c3cc(C=O)ccc32)CC1 ; Clc1ccccc1CC1CCNCC1 ; CC(C)(C)OC(=O)N1CCC(n2cc(-c3ccc(OC(F)(F)F)cc3)c3cc(CN4CCC(Cc5ccccc5Cl)CC4)ccc32)CC1 ; FC(F)(F)Oc1ccc(-c2cn(C3CCNCC3)c3ccc(CN4CCC(Cc5ccccc5Cl)CC4)cc23)cc1]

### Step 1: tert-butyl 4-indolin-1-ylpiperidine-1-carboxylate (07-37-1)

To a mixture of indoline (10.0 g, 83.9 mmol, 9.43 mL, 1.0 eq) and tert-butyl 4-oxopiperidine-1-carboxylate (21.7 g, 109.1 mmol, 1.30 eq) in MeOH (150 mL) was added Ti(i-PrO)₄ (23.8 g, 83.9 mmol, 24.8 mL, 1.0 eq). The mixture was stirred at 20° C. for 12 h under N₂. Then NaBH₃CN (10.5 g, 167.8 mmol, 2.0 eq) was added to the mixture portionwise. The resulting mixture was stirred at 20° C. for another 12 h, poured into H₂O (400 mL), filtered, and the solid was washed with DCM (100 mL*8). The filtrate was extracted with DCM (100 mL*2). The combined organic layers were washed with brine (500 mL), dried over anhydrous Na₂SO₄, filtered and concentrated under reduced pressure to give a residue. The residue was purified by column chromatography (SiO₂) to give compound 07-37-1 (13.0 g, 38.5 mmol, 46% yield). ¹H NMR (CDCl₃, 400 MHz): δ 7.11-7.02 (m, 2H), 6.67-6.59 (m, 1H), 6.44 (d, J=7.78 Hz, 1H), 4.27 (br s, 2H), 3.59-3.46 (nm, 1H), 3.36 (t, J=8.41 Hz, 2H), 2.96 (t, J=8.34 Hz, 2H), 2.88-2.70 (m, 2H), 1.81 (br d, J=12.80 Hz, 2H) 1.67-1.55 (m, 2H), 1.50 (s, 9H).

### Step 2: tert-butyl 4-(5-formylindolin-1-yl)piperidine-1-carboxylate (07-37-2)

To a solution of DMF (967 mg, 13.2 mmol, 1.02 mL, 2.0 eq) in ACN (20 mL) was added POCl₃ (2.03 g, 13.2 mmol, 1.23 mL, 2.0 eq) at 0° C. The mixture was stirred at 0° C. for 1 h under N₂. Then tert-butyl 4-indolin-1-ylpiperidine-1-carboxylate (2.00 g, 6.61 mmol, 1.0 eq) in DMF (5 mL) was added dropwise at 0° C. The mixture was stirred at 20° C. for another 6 h, and poured into ice water (200 mL). Then the aqueous phase was adjusted to pH 11 with solid NaOH, and extracted with DCM (80 mL*5). The combined organic layers were washed with brine (200 mL), dried over anhydrous Na₂SO₄, filtered and concentrated under reduced pressure to give a residue. The residue was purified by column chromatography (SiO₂) to give compound 07-37-2 (700 mg, 23% yield). ¹H NMR (CDCl₃, 400 MHz): δ 9.69-9.63 (m, 1H), 7.60-7.51 (m, 2H), 6.39 (d, J=8.66 Hz, 1H), 4.27 (br s, 2H), 3.65-3.54 (m, 3H), 3.04 (t, J=8.53 Hz, 2H), 2.80 (br t, J=12.23 Hz, 2H), 1.80 (br d, J=12.55 Hz, 2H), 1.67-1.61 (m, 2H), 1.48 (s, 9H). M+H⁺=331.2 (LCMS).

### Step 3: tert-butyl 4-(5-formylindol-1-yl)piperidine-1-carboxylate (07-37-3)

To a solution of 07-37-2 (500 mg, 1.0 eq) in DCM (10 mL) was added DDQ (524 mg, 2.31 mmol, 1.5 eq). The mixture was stirred at −78° C. for 1 h under N₂. The reaction mixture was poured into saturated sodium bicarbonate solution (100 mL) and extracted with DCM (40 mL*3). The combined organic layers were washed with brine (60 mL), dried over anhydrous Na₂SO₄, filtered, and concentrated under reduced pressure to give a residue. The residue was purified by column chromatography (SiO₂) to give compound 07-37-3 (300 mg, 817.8 μmol, 54% yield). ¹H NMR (CDCl₃, 400 MHz): δ 10.04 (s, 1H), 8.17 (d, J=1.13 Hz, 1H), 7.80 (dd, J=8.66, 1.51 Hz, 1H), 7.47 (d, J=8.66 Hz, 1H), 7.29 (d, J=3.39 Hz, 1H), 6.71 (d, J=3.26 Hz, 1H), 4.53-4.29 (m, 3H), 2.95 (br t, J=12.36 Hz, 2H), 2.10 (br d, J=12.30 Hz, 2H), 1.99-1.89 (m, 2H), 1.51 (s, 9H).

Steps 4-7 were carried out according to similar procedures as described in step 2 to step 5 of the synthesis of 07-2.

### 5-[[4-[(2-chlorophenyl)methyl]-1-piperidyl]methyl]-1-(4-piperidyl)-3-[4-(trifluoromethoxy)phenyl]indole (07-37)

M+H⁺=582.3 (LCMS). ¹H NMR (MeOD, 400 MHz): δ 8.14-8.04 (m, 1H), 7.83-7.73 (m, 4H), 7.47-7.32 (m, 4H), 7.28-7.15 (m, 3H), 4.98-4.88 (m, 1H), 4.40 (s, 2H), 3.64 (br d, J=12.57 Hz, 2H), 3.48 (br d, J=11.91 Hz, 2H), 3.42-3.33 (m, 2H), 3.03-2.89 (m, 2H), 2.74 (br d, J=6.39 Hz, 2H), 2.41-2.30 (m, 4H), 2.01 (br d, J=12.13 Hz, 1H), 1.91-1.82 (m, 2H), 1.65-1.49 (m, 2H).

The following compounds are synthesized in similar procedures as described above for the preparation of 07-37.

| Comp | | | Mass | 1H NMR (MeOD, |
|---|---|---|---|---|
| ID | Structure | Chemical Name | (M + H⁺) | 400 MHz) |
| 04-75 | 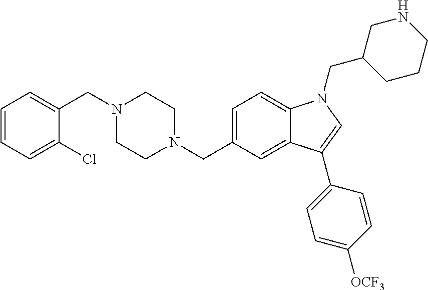 [SMILES: FC(F)(F)Oc1ccc(-c2cn(CC3CCCNC3)c3ccc(CN4CCN(Cc5ccccc5Cl)CC4)cc23)cc1] | 5-[[4-[(2- chlorophenyl) methyl] piperazin-1- yl]methyl]-1-(3- piperidyl methyl)-3-[4- (trifluoromethoxy) phenyl]indole dihydrochloride | Calc'd for C₃₃H₃₇Cl F₃N₄O: 597.3; Found: 597.3 | δ 8.19 (br s, 1H), 7.84 (dd, J = 8.71, 1.87 Hz, 2H), 7.78 (br s, 1H), 7.69 (t, J = 4.30 Hz, 2H), 7.59-7.54 (m, 1H), 7.53-7.43 (m, 3H), 7.36 (d, J = 8.38 Hz, 2H), 4.62 (br s, 4H), 4.30 (d, J = 7.06 Hz, 2H), 3.71 (br s, 8H), 3.35 (br d, J = 12.35 Hz, 1H), 3.19 (brd, J = 10.36 Hz, 1H), 2.98-2.83 (m, 2H), 2.45 (br s, 1H), 1.96 (br d, J = 14.33 Hz, 1H), 1.89-1.65 (m, 2H), 1.51-1.37 (m, 1H) |
| 07-17 | 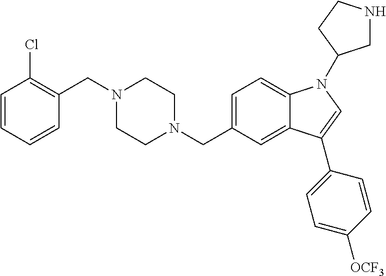 [SMILES: FC(F)(F)Oc1ccc(-c2cn(C3CCNC3)c3ccc(CN4CCN(Cc5ccccc5Cl)CC4)cc23)cc1] | 5-((4-(2- chlorobenzyl) piperazin-1- yl)methyl)-1- (pyrrolidin-3- yl)-3-(4- (trifluoromethoxy) phenyl)-1H- indole dihydrochloride | Calc'd for C₃₁H₃₃Cl F₃N₄O: 569.2; Found: 569.3 | δ 8.19 (s, 1H), 7.90- 7.83 (m, 3H), 7.78- 7.71 (m, 2H), 7.58- 7.54 (m, 1H), 7.54- 7.43 (m, 3 H), 7.36 (d, J = 7.94 Hz, 2H), 5.54 (quin, J = 7.11 Hz, 1H), 4.64-4.55 (m, 4H), 3.92 (dd, J = 12.57, 7.94 Hz, 1H), 3.75-3.53 (m, 11H), 2.77-2.65 (m, 1H), 2.60-2.50 (m, 1 H) |
| 07-9 | 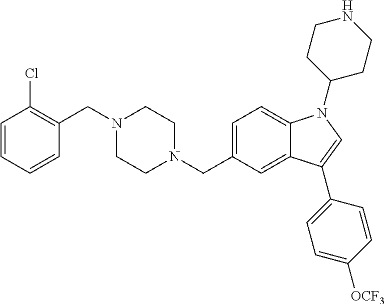 [SMILES: FC(F)(F)Oc1ccc(-c2cn(C3CCNCC3)c3ccc(CN4CCN(Cc5ccccc5Cl)CC4)cc23)cc1] | 5-((4-(2- chlorobenzyl) piperazin-1- yl)methyl)-1- (piperidin-4-yl)- 3-(4- (trifluoromethoxy) phenyl)-1H- indole dihydrochloride | Calc'd for C₃₂H₃₅Cl F₃N₄O: 583.2; Found: 583.3 | δ 8.13 (d, J = 1.3 Hz, 1H), 7.82-7.77 (m, 2H), 7.76-7.72 (m, 2H), 7.68 (dd, J = 2.0, 7.1 Hz, 1H), 7.53- 7.49 (m, 1H), 7.47- 7.42 (m, 2H), 7.42- 7.38 (m, 1H), 7.36- 7.32 (m, 2H), 4.56 (s, 2H), 4.39 (br s, 2H), 3.67-3.45 (m, 6H), 3.69-3.32 (m, 1H), 3.69-3.31 (m, 7H), 3.29 (td, J = 1.7, 3.3 Hz, 11H), 2.37- 2.25 (m, 4H) |

### Example 51: Synthesis of 3-[5-[[4-[(2-chlorophenyl)methyl]piperazin-1-yl]methyl]-3-(4-methoxyphenyl)indol-1-yl]-3-methyl-butan-1-amine (07-15)

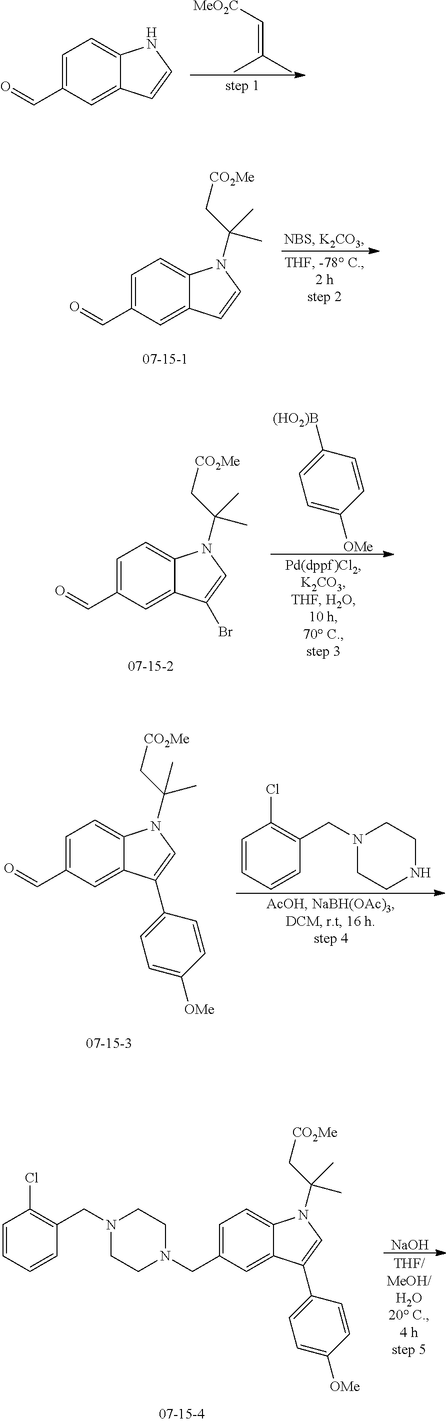

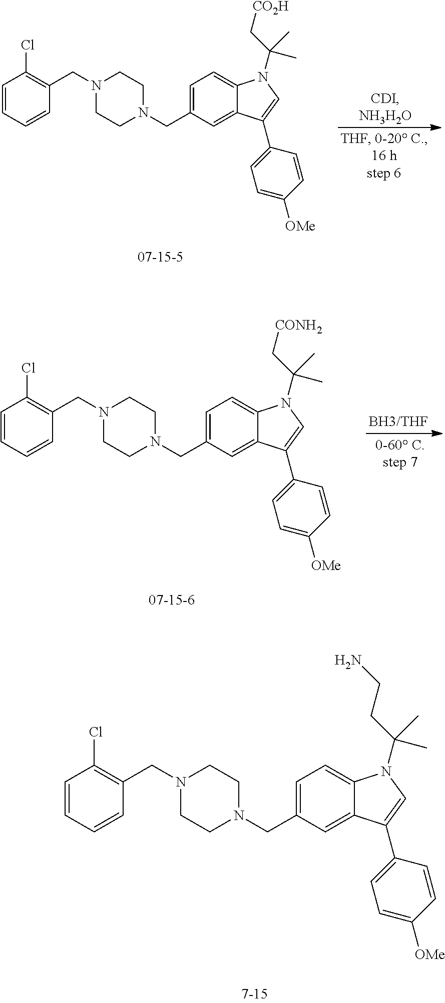 [SMILES: COc1ccc(-c2cn(C(C)(C)CC(=O)O)c3ccc(CN4CCN(Cc5ccccc5Cl)CC4)cc23)cc1 ; COc1ccc(-c2cn(C(C)(C)CC(N)=O)c3ccc(CN4CCN(Cc5ccccc5Cl)CC4)cc23)cc1 ; COc1ccc(-c2cn(C(C)(C)CCN)c3ccc(CN4CCN(Cc5ccccc5Cl)CC4)cc23)cc1]

### Step 1: methyl 3-(5-formylindol-1-yl)-3-methyl-butanoate (07-15-1)

To a solution of 1H-indole-5-carbaldehyde (10.0 g, 68.9 mmol, 1.0 eq) in methyl 3-methylbut-2-enoate (47.0 g, 411.9 mmol, 50.0 mL, 6.0 eq) and DCM (50 mL) was added t-BuOK (9.28 g, 82.7 mmol, 1.2 eq). After addition, the mixture was stirred at 80° C. for 18 h and concentrated. The residue was diluted with H₂O (40 mL) and extracted with EtOAc (350*2 mL). The combined organic layers were washed with H₂O (400 mL), brine (400 mL), dried over anhydrous Na₂SO₄, filtered, and concentrated. The residue was purified by column chromatography (SiO₂) and MPLC to give compound 07-15-1 (850 mg, 3.17 mmol, 5% yield). ¹H NMR (CDCl₃, 400 MHz): δ 10.01 (brs, 1H), 8.12-8.13 (d, J=0.8 Hz, 1H), 7.71-7.74 (m, 1H), 7.65-7.67 (m, 1H), 7.36-7.37 (m, 1H), 6.60-6.61 (m, 1H), 3.48 (s, 3H), 3.07 (s, 2H), 1.87 (s, 6H).

Steps 2-4 were carried out according to similar procedures as described in step 2-4 in the synthesis of 07-2.

### Step 4: methyl 3-[5-[[4-[(2-chlorophenyl)methyl]piperazin-1-yl]methyl]-3-(4-methoxy phenyl)indol-1-yl]-3-methyl-butanoate (07-11-4)

¹H NMR (CDCl₃, 400 MHz): δ 7.73 (s, 1H), 7.49-7.56 (m, 3H), 7.42-7.44 (m, 1H), 7.29-7.31 (m, 2H) 7.13-7.23 (m, 3H) 6.97-7.00 (m, 2H) 3.84 (s, 3H) 3.60-3.65 (m, 4H) 3.51-3.54 (m, 3H) 3.08 (s, 2H) 2.46-2.59 (m, 8H) 1.88 (s, 6H).

### Step 5: 3-[5-[[4-[(2-chlorophenyl)methyl]piperazin-1-yl]methyl]-3-(4-methoxy phenyl)indol-1-yl]-3-methyl-butanoic acid (07-11-5)

To a solution of 07-11-4 (300 mg, 535.6 μmol, 1.0 eq) in THF (3.0 mL)/MeOH (3.0 mL)/H₂O (1.50 mL) was added NaOH (64 mg, 1.61 mmol, 3.0 eq). The mixture was stirred at 20° C. for 4 h. The mixture was adjusted to pH=6.0 by addition of HCl aq (1N) and was concentrated to remove the organic solvents. The aqueous phase was extracted with DCM (15 mL*2). The combined organic layers were washed with H₂O (20 mL), brine (20 mL), dried over anhydrous Na₂SO₄, and concentrated under reduced pressure to give the crude product 07-11-5 (290 mg, 88% yield).

### Step 6: 3-[5-[[4-[(2-chlorophenyl)methyl]piperazin-1-yl]methyl]-3-(4-methoxyphenyl)indol-1-yl]-3-methyl-butanamide (07-15-6)

To a solution of 07-11-5 (260 mg, 1.0 eq) in THF (3 mL) was added CDI (231 mg, 1.43 mmol, 3.0 eq) at 0° C. After 4 h, ammonia (1.47 g, 30.3 mmol, 1.44 mL, 63.6 eq) was added drop-wise. The resulting mixture was stirred at 20° C. for another 1 h, diluted with H₂O (20 mL) and concentrated. The mixture was extracted with DCM (15 mL*2) and the combined organic layers were washed with H₂O (20 mL), dried over anhydrous Na₂SO₄, filtered, and concentrated under reduced pressure to give a residue. The residue was purified by prep-TLC (SiO₂) to give compound 07-15-6 (230 mg, 84% yield). ¹H NMR (CDCl₃, 400 MHz): δ 7.71 (s, 1H) 7.52-7.54 (d, J=8.0 Hz, 1H) 7.44-7.46 (m, 2H) 7.37-7.38 (m, 1H) 7.24-7.26 (m, 1H) 7.19 (m, 2H) 7.07-7.14 (m, 2H) 6.92-6.94 (m, 2H) 4.79 (m, 1H) 4.37 (m, 1H) 3.79 (s, 3H) 3.55-3.57 (m, 4H) 2.97 (s, 2H) 2.40-2.45 (m, 7H) 1.83 (m, 6H).

### Step 7: 3-[5-[[4-[(2-chlorophenyl)methyl]piperazin-1-yl]methyl]-3-(4-methoxy phenyl)indol-1-yl]-3-methyl-butan-1-amine (07-15)

To a solution of 07-15-6 (230 mg, 1.0 eq) in THF (5 mL) was added BH₃-Me₂S (10 M, 500.0 μL, 11.9 eq) at 0° C. After addition, the mixture was stirred at 0° C. for 0.5 h then heated to 60° C. for 5 h. The mixture was cooled to 20° C., quenched with H₂O (15 mL) at 20° C., and extracted with DCM (20 mL*2). The combined organic layers were washed with H₂O (20 mL), brine (20 mL), dried over anhydrous Na₂SO₄, filtered, and concentrated. The residue was redissolved in MeOH (2 mL) and aqueous HCl (1N, 5 mL). The mixture was stirred at 60° C. for 1 h, cooled to rt, adjusted to pH=9.0 with saturated Na₂CO₃ and extracted with DCM (20 mL*2). The combined organic layers were washed with brine (20 mL), dried over anhydrous Na₂SO₄, filtered, and concentrated. The residue was purified by prep-TLC (SiO₂) and prep-HPLC (FA condition) to give compound 07-15 as FA salt which was stirred with 1 N HCl aqueous (2 mL) for 1 h and then after lyophilization to give compound 07-15 (30 mg, 54.2 μmol, 13% yield, HCl). M+H⁺=531.3 (LCMS). ¹H NMR (MeOD, 400 MHz): δ 8.08 (s, 1H) 7.85-7.87 (d, J=8.0 Hz, 1H) 7.73-7.75 (m, 1H) 7.52-7.59 (m, 4H) 7.39-7.46 (m, 3H) 6.97-7.00 (m, 2H) 4.57 (s, 2H) 3.81 (s, 3H) 3.65 (m, 8H) 2.56-2.61 (m, 2H) 2.49-2.55 (m, 2H) 1.83 (s, 6H).

### Example 52: Synthesis of (1R)-3-[(1S)-5-[[4-[(2-chlorophenyl)methyl]piperazin-1-yl]methyl]-3-[4-(trifluoromethoxy)phenyl]indol-1-yl]cyclopentanamine (07-40)

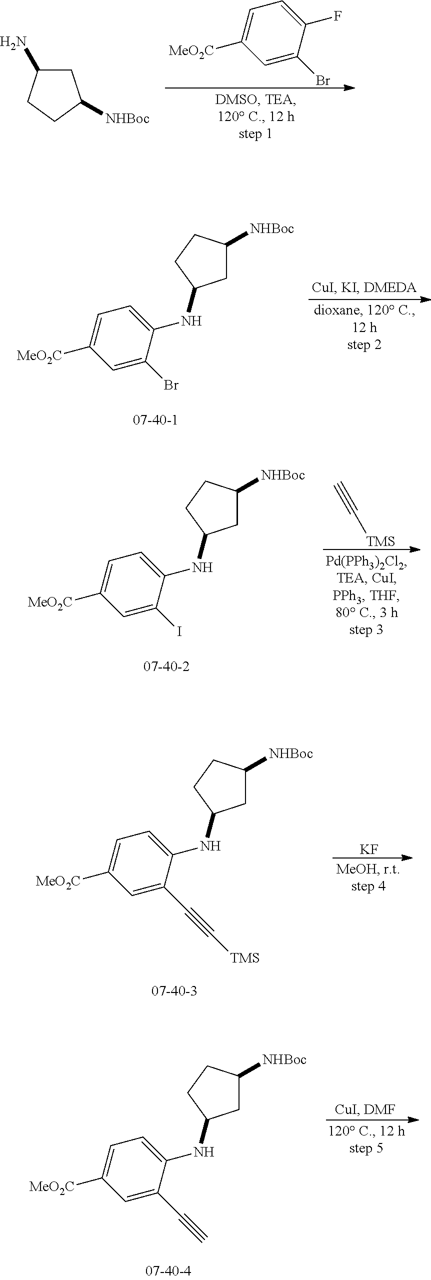

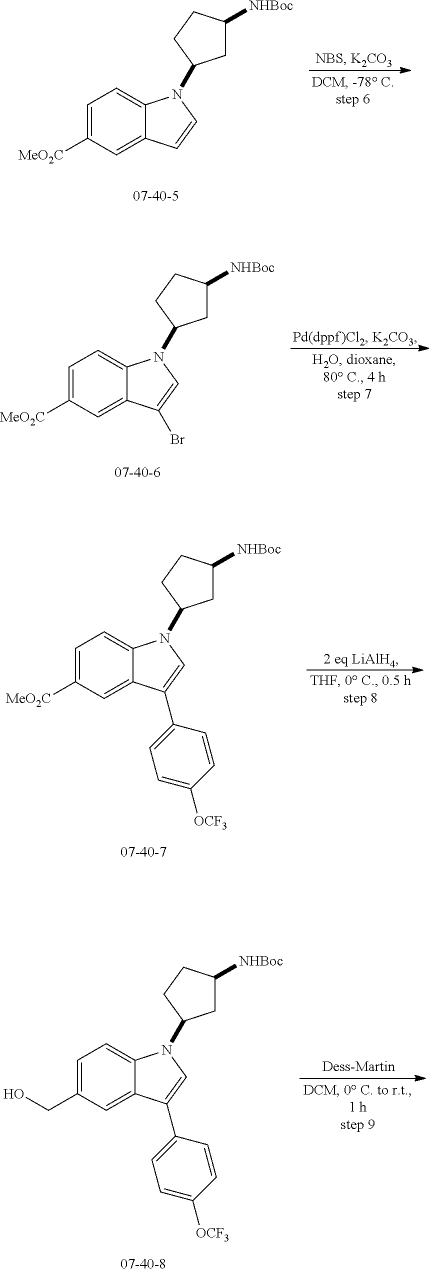

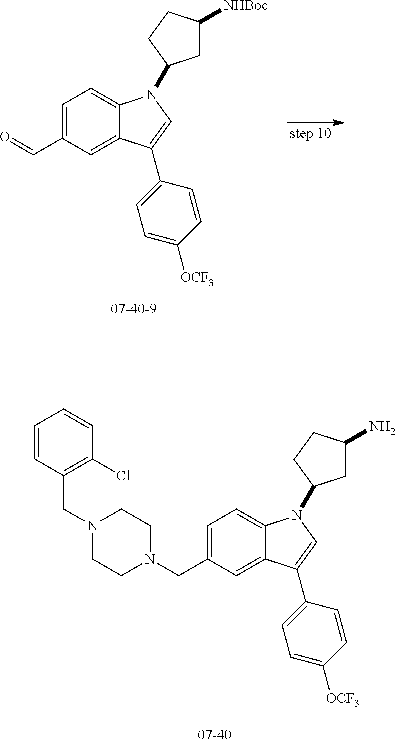 [SMILES: CC(C)(C)OC(=O)N[C@@H]1CC[C@H](n2cc(-c3ccc(OC(F)(F)F)cc3)c3cc(C=O)ccc32)C1 ; N[C@@H]1CC[C@H](n2cc(-c3ccc(OC(F)(F)F)cc3)c3cc(CN4CCN(Cc5ccccc5Cl)CC4)ccc32)C1]

### Step 1: methyl 3-bromo-4-[[(3R)-3-(tert-butoxycarbonylamino)cyclopentyl]amino]benzoate (07-40-1)

To a solution of tert-butyl N-[(1S)-3-aminocyclopentyl]carbamate (12.0 g, 59.9 mmol, 1.0 eq) and methyl 3-bromo-4-fluoro-benzoate (13.9 g, 59.9 mmol, 1.0 eq) in DMSO (150 mL) was added DIPEA (15.5 g, 119.8 mmol, 20.9 mL, 2.0 eq). The mixture was stirred at 120° C. for 12 h, poured into H₂O (250 mL) and extracted with EtOAc (80 mL*3). The combined organic layers were washed with brine (120 mL), dried over anhydrous Na₂SO₄, filtered, and concentrated under reduced pressure to give a residue. The residue was purified by column chromatography (SiO₂) to give compound 07-40-1 (16.0 g, 36.8 mmol, 61% yield). M+H⁺=413.2 (LCMS). ¹H NMR (CDCl₃, 400 MHz): δ 8.12 (d, J=1.88 Hz, 1H), 7.85 (dd, J=8.60, 1.57 Hz, 1H), 6.58 (d, J=8.66 Hz, 1H), 4.88 (br d, J=4.64 Hz, 1H), 4.63 (br s, 1H), 4.09-3.99 (m, 1H), 3.86 (s, 3H), 2.62-2.51 (m, 1H), 2.15-2.04 (m, 2H), 1.75-1.63 (m, 2H), 1.45 (s, 9H).

### Step 2: methyl 4-[[(3R)-3-(tert-butoxycarbonylamino)cyclopentyl]amino]-3-iodo-benzoate (07-40-2)

To a solution of compound 07-40-1 (8.00 g, 19.4 mmol, 1.0 eq) in dioxane (100 mL) were added N,N′-dimethylethane-1,2-diamine (341 mg, 3.87 mmol, 416.2 μL, 0.2 eq), KI (6.43 g, 38.7 mmol, 2.0 eq) and CuI (368 mg, 1.94 mmol, 0.1 eq). The mixture was stirred at 120° C. for 12 h, cooled to rt, poured into H₂O (250 mL) and extracted with EtOAc (80 mL*3). The combined organic layers were washed with brine (120 mL), dried over anhydrous Na₂SO₄, filtered, and concentrated under reduced pressure to give compound 07-40-2 (6.00 g, crude). M+H⁺=461.2 (LCMS).

### Step 3: methyl 4-[[(3R)-3-(tert-butoxycarbonylamino)cyclopentyl]amino]-3-(2-trimethylsilylethynyl)benzoate (07-40-3)

To a solution of compound 07-40-2 (2.00 g, 4.34 mmol, 1.0 eq) and ethynyl(trimethyl)silane (1.28 g, 13.0 mmol, 1.80 mL, 3.0 eq) in THF (30 mL) were added Pd(PPh₃)₂Cl₂ (304.9 mg, 434.4 μmol, 0.1 eq), PPh₃ (148.1 mg, 564.8 μmol, 0.13 eq), TEA (10.9 g, 108.2 mmol, 15.0 mL, 24.9 eq) and CuI (413.74 mg, 2.17 mmol, 0.5 eq). The mixture was stirred at 80° C. for 12 h under N₂. The reaction mixture was cooled to room temperature, poured into H₂O (150 mL), and extracted with EtOAc (50 mL*3). The combined organic layers were washed with brine (80 mL), dried over anhydrous Na₂SO₄, filtered, and concentrated under reduced pressure to give compound 07-40-3 (4.50 g, crude). M+H⁺=431.2 (LCMS).

### Step 4: methyl 4-[[(3R)-3-(tert-butoxycarbonylamino)cyclopentyl]amino]-3-ethynyl-benzoate (07-40-4)

To a solution of 07-40-3 (7.00 g, 16.8 mmol, 1.0 eq) in MeOH (30 mL) was added KF (2.93 g, 50.4 mmol, 1.18 mL, 3.0 eq). After 2 h, the reaction mixture was concentrated under reduced pressure. The residue was poured into H₂O (150 mL) and extracted with EtOAc (50 mL*3). The combined organic layers were washed with brine (80 mL), dried over anhydrous Na₂SO₄, filtered, and concentrated. The crude product was purified by column chromatography (SiO₂) to give compound 07-40-4 (3.30 g, 51% yield). M+H⁺=359.2.

### Step 5: methyl (1S)-1-[(3R)-3-(tert-butoxycarbonylamino)cyclopentyl]indole-5-carboxylate (07-40-5)

To a solution of compound 07-40-4 (3.30 g, 1.0 eq) in DMF (40 mL) was added CuI (350 mg, 1.84 mmol, 0.2 eq). The mixture was stirred at 120° C. for 12 h, cooled to room temperature, poured into H₂O (80 mL) and extracted with EtOAc (30 mL*3). The combined organic layers were washed with brine (50 mL), dried over anhydrous Na₂SO₄, filtered and concentrated to give a residue, which was purified by column chromatography (SiO₂) to give compound 07-40-5 (3.00 g, 7.32 mmol, 80% yield). ¹H NMR (CDCl₃, 400 MHz): δ 8.39 (d, J=1.13 Hz, 1H), 7.91 (dd, J=8.66, 1.51 Hz, 1H), 7.37 (d, J=8.78 Hz, 1H), 7.29 (d, J=3.26 Hz, 1H), 6.63 (d, J=3.14 Hz, 1H), 4.83 (quin, J=7.91 Hz, 1H), 4.21-4.06 (m, 1H), 3.94 (s, 3H), 2.79-2.65 (m, 1H), 2.34-2.19 (m, 2H), 2.11-2.04 (m, 1H), 1.86-1.73 (m, 1H), 1.74-1.73 (m, 1H), 1.46 (s, 9H).

### Step 6: methyl (1S)-3-bromo-1-[(3R)-3-(tert-butoxycarbonylamino)cyclopentyl]indole-5-carboxylate (07-40-6)

To a solution of 07-40-5 (600 mg, 1.67 mmol, 1.0 eq) in DCM (6 mL) was added NBS (267 mg, 1.50 mmol, 0.9 eq) and K₂CO₃ (462 mg, 3.34 mmol, 2.0 eq) at −78° C. After 1 h, the reaction mixture was poured into H₂O (80 mL) and extracted with DCM (30 mL*3). The combined organic layers were washed with brine (50 mL), dried over anhydrous Na₂SO₄, filtered and concentrated under reduced pressure to give compound 07-40-6 (750 mg, crude). M+H⁺=437.1 (LCMS).

### Step 7: methyl (1S)-1-[(3R)-3-(tert-butoxycarbonylamino)cyclopentyl]-3-[4-(trifluoromethoxy)phenyl]indole-5-carboxylate (07-40-7)

To a solution of 07-40-6 (750 mg, 1.77 mmol, 1.0 eq) and [4-(trifluoromethoxy)phenyl]boronic acid (546 mg, 2.66 mmol, 1.5 eq) in dioxane (10 mL) and H₂O (1 mL) was added K₂CO₃ (489 mg, 3.54 mmol, 2.0 eq) and Pd(dppf)Cl₂ (129 mg, 177.0 μmol, 0.1 eq). The mixture was stirred at 80° C. for 12 h under N₂, cooled to room temperature and poured into H₂O (100 mL). The mixture was extracted with EtOAc (40 mL*3) and the combined organic layers were washed with brine (60 mL), dried over anhydrous Na₂SO₄, filtered and concentrated under reduced pressure to give a residue. The residue was purified by column chromatography (SiO₂) to give compound 07-40-7 (550 mg, 932.1 μmol, 53% yield). ¹H NMR (CDCl₃, 400 MHz): δ 8.52 (br s, 1H), 7.90 (br d, J=8.16 Hz, 1H), 7.58 (br d, J=7.40 Hz, 2H), 7.36 (br s, 2H), 7.24 (br d, J=7.65 Hz, 2H), 4.88-4.72 (m, 1H), 4.06 (br s, 1H), 3.87 (br s, 3H), 2.37 (br d, J=5.90 Hz, 1H), 2.34-2.23 (m, 1H), 2.19-2.02 (m, 2H), 1.88-1.78 (m, 1H), 1.73 (br s, 1H), 1.38 (br s, 9H).

### Step 8: tert-butyl N-[(1R)-3-[(1S)-5-(hydroxymethyl)-3-[4-(trifluoromethoxy)phenyl]indol-1-yl]cyclopentyl]carbamate (07-40-8)

To a solution of 07-40-7 (200 mg, 385.7 μmol, 1.0 eq) in THF (5 mL) was added LiAlH₄ (29 mg, 771 μmol, 2.0 eq). The mixture was stirred at 0° C. for 40 min under N₂, poured into H₂O (50 mL) and extracted with EtOAc (20 mL*3). The combined organic layers were washed with brine (30 mL), dried over anhydrous Na₂SO₄, filtered and concentrated under reduced pressure to give compound 07-40-8 (220 mg, crude). M−H₂O+H⁺=473.3 (LCMS).

### Step 9: tert-butyl N-[(1R)-3-[(1S)-5-formyl-3-[4-(trifluoromethoxy)phenyl]indol-1-yl]cyclopentyl]carbamate (07-40-9)

To a solution of 07-40-8 (180 mg, 377.7 μmol, 1.0 eq) in DCM (6 mL) was added Dess-Martin (160 mg, 377.8 μmol, 116.9 μL, 1.0 eq). The mixture was stirred at 0° C. for 40 min under N₂, filtered and the solid was washed with DCM (20 mL*3). The filtrate was concentrated under reduced pressure to give a crude product which was purified by column chromatography (SiO₂) to give compound 07-40-9 (180 mg, 309.8 μmol, 82% yield). M+H⁺=489.3 (LCMS).

### Step 10: (1R)-3-[(1S)-5-[[4-[(2-chlorophenyl)methyl]piperazin-1-yl]methyl]-3-[4-(trifluoromethoxy)phenyl]indol-1-yl]cyclopentanamine (07-40)

Compound 07-40 was prepared from compound 07-40-9 according to the procedures described in the steps 4 and 5 in the synthesis of compound 07-2. M+H⁺=583.3 (LCMS). ¹H NMR (MeOD, 400 MHz): δ 8.15 (d, J=1.10 Hz, 1H), 7.85-7.80 (m, 3H), 7.75-7.68 (m, 2H), 7.56-7.53 (m, 1H), 7.50-7.43 (m, 3H), 7.36 (d, J=7.94 Hz, 2H), 5.16-5.07 (m, 1H), 4.59 (s, 2H), 4.49 (br s, 2H), 3.88-3.79 (m, 1H), 3.71-3.46 (m, 8H), 2.85-2.77 (m, 1H), 2.40-2.27 (m, 3H), 2.09-2.00 (m, 2H).

### Example 53: Synthesis of N-[[1-(3-aminopropyl)-3-[4-(trifluoromethoxy)phenyl]indol-5-yl]methyl]-4-(2-chlorophenyl)-N-methyl-butan-1-amine (08-8)

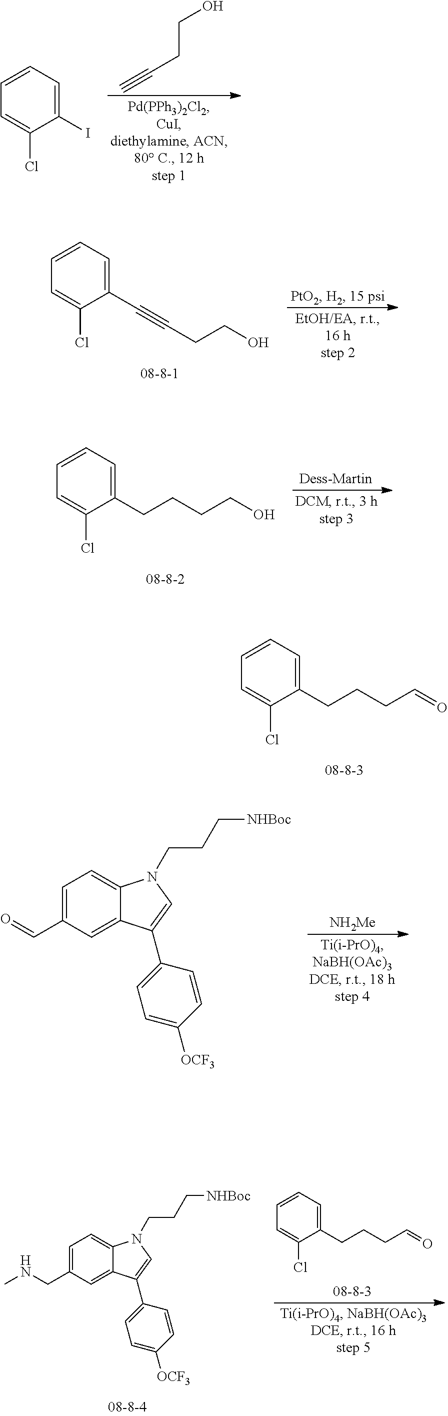

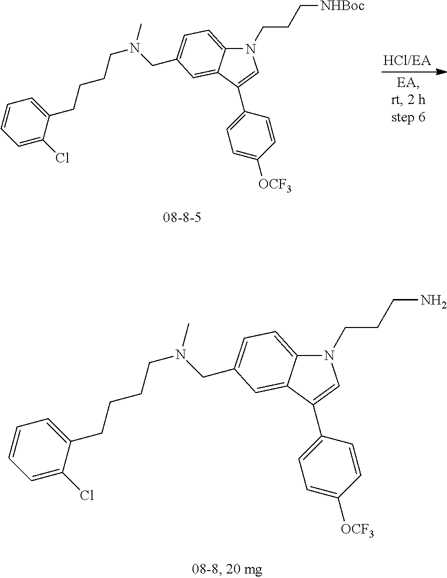 [SMILES: CN(CCCCc1ccccc1Cl)Cc1ccc2c(c1)c(-c1ccc(OC(F)(F)F)cc1)cn2CCCNC(=O)OC(C)(C)C ; CN(CCCCc1ccccc1Cl)Cc1ccc2c(c1)c(-c1ccc(OC(F)(F)F)cc1)cn2CCCN]

### Step 1: 4-(2-chlorophenyl)but-3-yn-1-ol (08-8-1)

To a mixture of 1-chloro-2-iodo-benzene (2.00 g, 8.39 mmol, 1.0 eq) in THF (20 mL) were added but-3-yn-1-ol (587 mg, 8.39 mmol, 632.1 μL, 1.0 eq), Pd(PPh₃)₂Cl₂ (588 mg, 839.0 μmol, 0.1 eq), CuI (159 mg, 839.0 μmol, 0.1 eq) and Et₃N (10 mL). The mixture was degassed and purged with N₂ three times, and stirred at 80° C. for 12 h under N₂. The mixture was cooled to room temperature, poured to water (30 mL), and extracted with EtOAc (30 mL*2). The combined organic layers were washed with brine (30 mL*2), dried over anhydrous Na₂SO₄, filtered and concentrated under reduced pressure to give a residue. The residue was purified by column chromatography (SiO₂) to give compound 08-8-1 (1.20 g, 6.05 mmol, 72% yield).

### Step 2: 4-(2-chlorophenyl)butan-1-ol (08-8-2)

A mixture of 08-8-1 (1.20 g, 6.64 mmol, 1.0 eq), PtO₂ (600 mg, 2.64 mmol, 0.40 eq) in EtOH (25 mL) and EtOAc (25 mL) was degassed and purged with H₂ thrice, and stirred at 20° C. for 16 h under H₂ (15 psi). The mixture was filtered and the filtrate was concentrated under reduced pressure to give compound 08-8-2 (1.20 g, 49% yield) which was used into the next step without further purification. ¹H NMR (CDCl₃, 400 MHz): δ 7.24-7.26 (m, 1H) 7.04-7.13 (m, 3H) 3.58-3.65 (m, 2H) 2.63-2.70 (m, 2H) 1.55-1.65 (m, 5H).

### Step 3: 4-(2-chlorophenyl)butanal (08-8-3)

To a solution of 08-8-2 (500 mg, 1.0 eq) in DCM (2 mL) was added Dess-Martin (1.15 g, 2.71 mmol, 839.0 μL, 1.0 eq) at 0° C. After addition, the mixture was stirred at 20° C. for 3 h, diluted with DCM (20 mL) and stirred for 0.5 h. The mixture was filtered, and the filtrate was washed with H₂O (20 mL), brine (20 mL), dried over anhydrous Na₂SO₄ and concentrated under reduced pressure to give a residue. The residue was purified by column chromatography (SiO₂) to give compound 08-8-3 (350.0 mg, 1.92 mmol, 71% yield). ¹H NMR (CDCl₃, 400 MHz): δ 9.71 (brs, 1H) 7.26-7.28 (m, 1H) 7.05-7.14 (m, 3H) 2.69-2.72 (m, 2H) 2.40-2.44 (m, 2H) 1.86-1.97 (m, 2H).

### Step 4: tert-butyl N-[3-[5-(methylaminomethyl)-3-[4-(trifluoromethoxy)phenyl]indol-1-yl]propyl]carbamate (08-8-4)

To a solution of tert-butyl N-[3-[5-formyl-3-[4-(trifluoromethoxy)phenyl]indol-1-yl]propyl]carbamate (200 mg, 432.5 μmol, 1.0 eq) in MeOH (6.00 mL) was added Ti(i-PrO)₄ (184 mg, 648.7 μmol, 192.0 μL, 1.5 eq) and methanamine (2 M, 500.0 μL, 2.3 eq). After 16 h, NaBH₃CN (54 mg, 864.9 μmol, 2.0 eq) was added in one portion. The resulting mixture was stirred at 20° C. for another 2 h, quenched with H₂O (0.5 mL) and concentrated under reduced pressure. The residue was diluted with DCM (20 mL), stirred for 10 min and filtered. The filtrate was concentrated under reduced pressure to give a residue which was purified by prep-TLC (SiO₂) to give compound 08-8-4 (70 mg, 23% yield). M+H⁺=478.3 (LCMS).

### Step 5: tert-butyl N-[3-[5-[[4-(2-chlorophenyl)butyl-methyl-amino]methyl]-3-[4-(trifluoromethoxy)phenyl]indol-1-yl]propyl]carbamate (08-8-5)

To a solution of 08-8-4 (85 mg, 1.0 eq) in MeOH (2 mL) were added Ti(i-PrO)₄ (75.8 mg, 267.0 μmol, 79.0 μL, 1.5 eq) and 08-8-3 (35.7 mg, 195.8 μmol, 1.1 eq). The mixture was stirred at 20° C. for 12 h, then NaBH₃CN (22.3 mg, 356.0 μmol, 2.0 eq) was added in one portion. The mixture was stirred at 20° C. for another 4 h. Water (0.1 mL) was added and the mixture was stirred for 10 min, filtered and the filtrate was concentrated under reduced pressure. The residue was purified by prep-HPLC (FA condition) to give compound 08-8-5 (41 mg, 33% yield, FA)

### Step 6: N-[[1-(3-aminopropyl)-3-[4-(trifluoromethoxy)phenyl]indol-5-yl]methyl]-4-(2-chlorophenyl)-N-methyl-butan-1-amine (08-8)

To a solution of 08-8-5 (41 mg, 1.0 eq) in EtOAc (1 mL) was added HCl/EtOAc (2.32 mg, 63.6 μmol, 1.0 mL, 1.0 eq) dropwise slowly. The mixture was stirred at 20° C. for 2 h, filtered and the collected solid was washed with EtOAc (5 mL*2), and dried to give compound 08-8 (21 mg, 32.6 μmol, 51% yield, HCl). M+H⁺=544.3 (LCMS). ¹H NMR (MeOD, 400 MHz): δ 8.06 (s, 1H) 7.76-7.78 (m, 2H) 7.70 (s, 1H) 7.65-7.67 (m, 1H) 7.37-7.39 (m, 1H) 7.27-7.34 (m, 3H) 7.11-7.20 (m, 3H) 4.34-4.53 (m, 4H) 3.28-3.29 (m, 1H) 3.05-3.13 (m, 1H)) 2.94-2.98 (m, 2H) 2.67-2.77 (m, 5H) 2.22-2.26 (m, 2H) 1.77-1.85 (m, 2H) 1.62-1.68 (m, 2H).

### Example 54: Synthesis of [1-(3-aminopropyl)-3-[4-(trifluoromethoxy)phenyl]indol-5-yl]-[4-[(2-chlorophenyl)methyl]piperazin-1-yl]methanone (08-9)

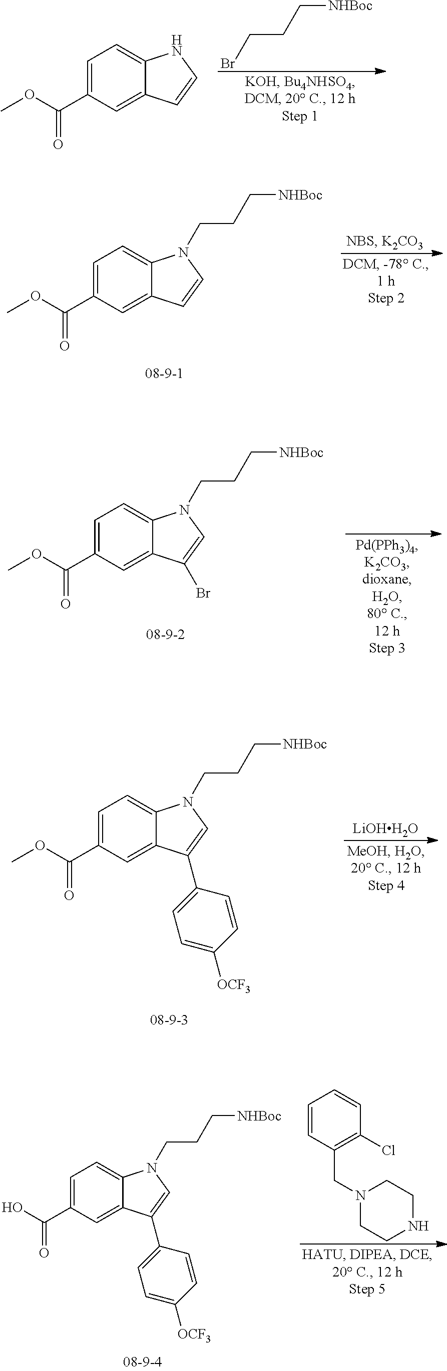

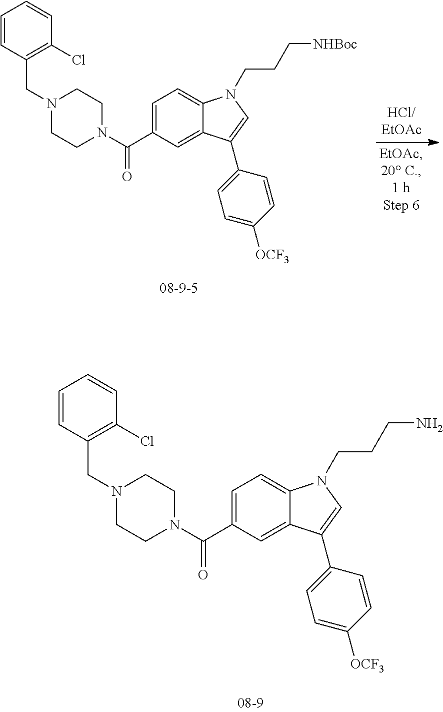 [SMILES: CC(C)(C)OC(=O)NCCCn1cc(-c2ccc(OC(F)(F)F)cc2)c2cc(C(=O)N3CCN(Cc4ccccc4Cl)CC3)ccc21 ; NCCCn1cc(-c2ccc(OC(F)(F)F)cc2)c2cc(C(=O)N3CCN(Cc4ccccc4Cl)CC3)ccc21]

### Step 3: methyl 1-[3-(tert-butoxycarbonylamino)propyl]-3-[4-(trifluoromethoxy)phenyl]indole-5-carboxylate (08-9-3)

The title compound was prepared from methyl 1H-indole-5-carboxylate according to similar procedures as described in step 1 to 3 in the synthesis of 04-1.

### Step 4: 1-[3-(tert-butoxycarbonylamino)propyl]-3-[4-(trifluoromethoxy)phenyl]indole-5-carboxylic acid (08-9-4)

To a mixture of 08-9-3 (2.00 g, 4.06 mmol, 1.0 eq) in MeOH (20 mL) and H₂O (6 mL) was added LiOH*H₂O (852 mg, 20.3 mmol, 5.0 eq). The mixture was stirred at 20° C. for 12 h, diluted with aqueous HCl (0.5 M) (40 mL) and extracted with EtOAc (20 mL*3). The combined organic layers were washed with brine (20 mL), dried over anhydrous Na₂SO₄, filtered and concentrated under reduced pressure to give compound 08-9-4 (1.50 g, crude).

### Step 5: tert-butyl N-[3-[5-[4-[(2-chlorophenyl)methyl]piperazine-1-carbonyl]-3-[4-(trifluoromethoxy)phenyl]indol-1-yl]propyl]carbamate (08-9-5)

To a mixture of 08-9-4 (150 mg, 313.5 μmol, 1.0 eq) in DCE (5 mL) were added HATU (119 mg, 313.5 μmol, 1.0 eq), DIPEA (121. mg, 940.5 μmol, 164.2 μL, 3.0 eq) and 1-[(2-chlorophenyl)methyl]piperazine (66 mg, 313.5 μmol, 1.0 eq). The mixture was stirred at 20° C. for 12 h, diluted with aqueous of NaHCO₃(1 M, 5 mL) and extracted with DCM (20 mL). The combined organic layers were washed with brine (5 mL), dried over anhydrous Na₂SO₄, filtered and concentrated. The residue was purified by prep-TLC (SiO₂) to give compound 08-9-5 (50 mg, 22% yield).

### Step 6: [1-(3-aminopropyl)-3-[4-(trifluoromethoxy)phenyl]indol-5-yl]-[4-[(2-chlorophenyl)methyl]piperazin-1-yl]methanone (08-9)

To a mixture of 08-9-5 (50.0 mg, 1.0 eq) in EtOAc (1 mL) was added HCl/EtOAc (4 M, 3.0 mL). The mixture was stirred at 20° C. for 1 h and concentrated under reduced pressure to give compound 08-9 (27 mg, 40.5 μmol, 55% yield, HCl). M+H⁺=571.3 (LCMS). ¹H NMR (MeOD, 400 MHz): δ 7.95 (s, 1H), 7.67-7.64 (m, 4H), 7.63 (d, J=5.2 Hz, 1H), 7.58 (d, J=8.4 Hz, 1H), 7.48-7.38 (m, 1H), 7.39-7.37 (m, 1H), 7.69 (d, J=8.0 Hz, 2H), 4.50 (s, 2H), 4.33 (t, J=5.6 Hz, 2H), 3.60-3.26 (m, 8H), 2.87 (t, J=7.2 Hz, 2H), 2.19-2.12 (m, 2H).

### Example 55: Synthesis of 1-(3-aminopropyl)-N-[1-[(2-chlorophenyl)methyl]-4-piperidyl]-3-[4-(trifluoromethoxy)phenyl]indol-5-amine (08-12)

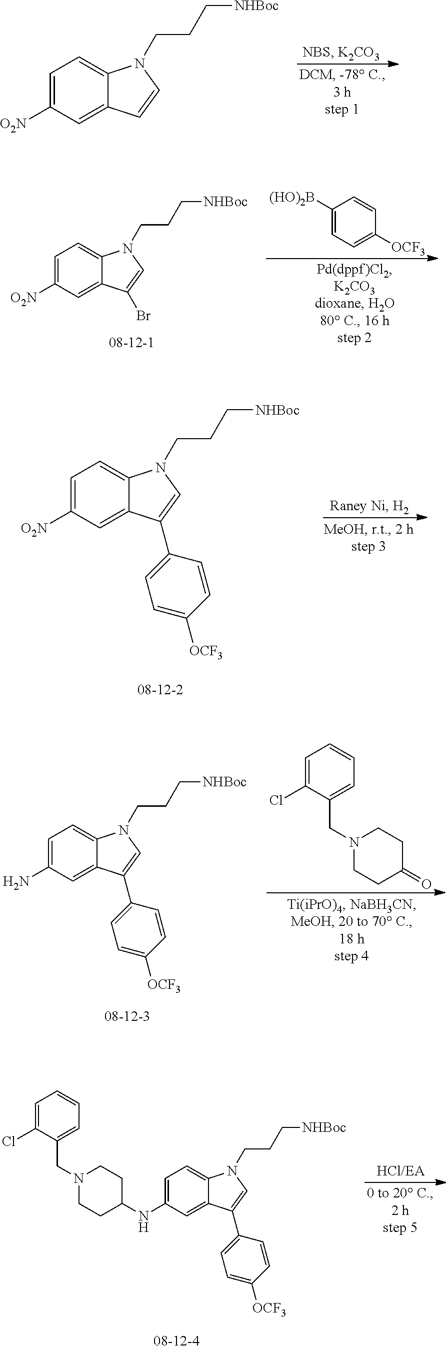

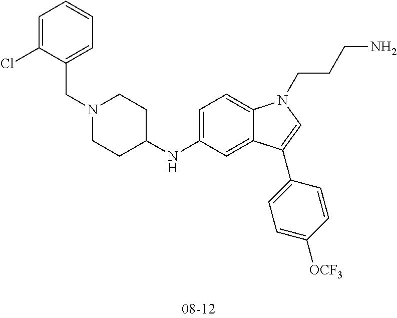 [SMILES: NCCCn1cc(-c2ccc(OC(F)(F)F)cc2)c2cc(NC3CCN(Cc4ccccc4Cl)CC3)ccc21]

### Step 1: tert-butyl N-[3-[5-nitro-3-[4-(trifluoromethoxy)phenyl]indol-1-yl]propyl]carbamate (08-12-1)

To a solution of tert-butyl N-[3-(5-nitroindol-1-yl)propyl]carbamate (2.00 g, 6.26 mmol, 1.0 eq) in DCM (50 mL) was added NBS (1.11 g, 6.26 mmol, 1.0 eq) and K₂CO₃ (1.04 g, 7.51 mmol, 1.2 eq). The mixture was stirred at −78° C. for 3 h, poured into saturated Na₂SO₃ aq (50 mL), and extracted with DCM (50 mL). The organic layers were washed with H₂O (10 mL), brine (10 mL), dried over anhydrous Na₂SO₄, filtered and the filtrate was concentrated under reduced pressure to give a crude compound 08-12-1 (2.10 g, 5.27 mmol, 84% yield) which was used directly in the next step. ¹H NMR (MeOD, 400 MHz): δ 8.46 (s, 1H) 8.06-8.09 (dd, J=9.2 Hz, 2.4 Hz, 1H) 7.28 (d, J=9.2 Hz, 1H) 7.19 (s, 1H) 4.50 (brs, 1H) 4.13-4.16 (t, J=7.2 Hz, 2H) 3.08-3.10 (m, 2H) 1.94-2.01 (m, 2H) 1.38 (s, 9H).

### Step 2: tert-butyl N-[3-[5-nitro-3-[4-(trifluoromethoxy)phenyl]indol-1-yl]propyl]carbamate (08-12-2)

A mixture of 08-12-1 (500 mg, 1.26 mmol, 1.0 eq), [4-(trifluoromethoxy)phenyl]boronic acid (311 mg, 1.51 mmol, 1.20 eq), Pd(dppf)Cl₂ (46.1 mg, 63.0 μmol, 0.05 eq) and K₂CO₃ (435 mg, 3.15 mmol, 2.5 eq) in dioxane (8 mL) and H₂O (2 mL) was degassed and purged with N₂, and the mixture was stirred at 80° C. for 16 h under N₂. The mixture was cooled to room temperature, diluted with H₂O (25 mL), and extracted with EtOAc (20 mL*2). The combined organic layers were washed with H₂O (25 mL), brine (25 mL), dried over anhydrous Na₂SO₄ and filtered. The filtrate was concentrated to give a residue which was purified by column chromatography (SiO₂) to give compound 08-12-2 (450 mg, 938.6 μmol, 75% yield).

### Step 3: tert-butyl N-[3-[5-amino-3-[4-(trifluoromethoxy)phenyl]indol-1-yl]propyl]carbamate (08-12-3)

To a solution of compound 08-12-2 (450 mg, 938.6 μmol, 1.0 eq) in EtOH (20 mL) was added Raney-Ni (0.45 g). The suspension was degassed and purged with H₂ thrice and the mixture was stirred under H₂ (15 psi) at 20° C. for 4 h, filtered, and the filtrate was concentrated under reduced pressure to give compound 08-12-3 (300 mg, 667.4 μmol, 71% yield). ¹H NMR (MeOD, 400 MHz): δ 7.51 (d, J=8.8 Hz, 1H) 7.17-7.19 (m, 5H) 6.53-6.79 (m, 1H) 4.43 (brs, 1H) 4.06-4.09 (m, 2H) 3.07-3.08 (m, 2H) 1.93-2.00 (m, 2H) 1.36 (s, 9H).

### Step 4: tert-butyl N-[3-[5-[[1-[(2-chlorophenyl)methyl]-4-piperidyl]amino]-3-[4-(trifluoromethoxy)phenyl]indol-1-yl]propyl]carbamate (08-12-4)

To a solution of compound 08-12-3 (295 mg, 656.3 μmol, 1.0 eq) in MeOH (12 mL) were added 1-[(2-chlorophenyl)methyl]piperidin-4-one (176 mg, 787.56 μmol, 1.2 eq) and Ti(i-PrO)₄ (379 mg, 1.33 mmol, 395.2 μL, 1.5 eq). The mixture was stirred at 80° C. for 12 h, cooled to 20° C. and NaBH₃CN (82 mg, 1.31 mmol, 2.0 eq) was added in one portion. The resulting mixture was stirred at 20° C. for 6 h, diluted with DCM (30 mL) and stirred for 10 min, and filtered. The filtrate was washed with H₂O (20 mL), brine (20 mL), dried over anhydrous Na₂SO₄, filtered and concentrated. The residue was purified by column chromatography (SiO₂) to give compound 08-12-5 (211.0 mg, 46% yield). ¹H NMR (CDCl₃, 400 MHz): δ 7.51-7.53 (m, 2H) 7.40 (m, 1H) 7.26 (m, 1H) 7.20 (m, 1H) 7.17-18 (m, 3H) 7.01-7.12 (m, 3H) 7.00 (s, 1H) 6.63 (d, J=2.0 Hz, 1H) 4.40 (brs, 1H) 4.02-4.08 (m, 2H) 3.57 (s, 2H) 3.28-3.30 (m, 1H) 3.07-3.08 (m, 2H) 2.80-2.83 (m, 2H) 2.20 (m, 2H) 2.00-2.03 (m, 2H) 1.95-1.98 (m, 2H) 1.47 (m, 1H) 1.36 (s, 10H).

### Step 6: 1-(3-aminopropyl)-N-[1-[(2-chlorophenyl)methyl]-4-piperidyl]-3-[4-(trifluoromethoxy)phenyl]indol-5-amine (08-12)

To a solution of compound 08-12-5 (210 mg, 1.0 eq) in EtOAc (5 mL) was added HCl/EtOAc (4 M, 2.0 mL, 25.0 eq) dropwise at 0° C. After addition, the mixture was stirred at 20° C. for 2 h. The mixture was filtered and the collected solid was washed with EtOAc (2 mL*2), and dried to give compound 08-12 (139 mg, 220.6 μmol, 69% yield, HCl). M+H⁺=557.3 (LCMS). ¹H NMR (MeOD, 400 MHz): δ 8.08 (s, 1H) 7.75-7.81 (m, 5H) 7.46-7.48 (m, 1H) 7.37-7.39 (m, 5H) 4.53 (s, 2H) 4.43-4.47 (m, 2H) 3.99 (m, 1H) 3.67-3.70 (m, 2H) 3.31-3.33 (m, 2H) 2.96-3.00 (m, 2H) 2.20-2.27 (m, 6H).

### Example 56: Conservation of Binding Sites Across the Ras Superfamily

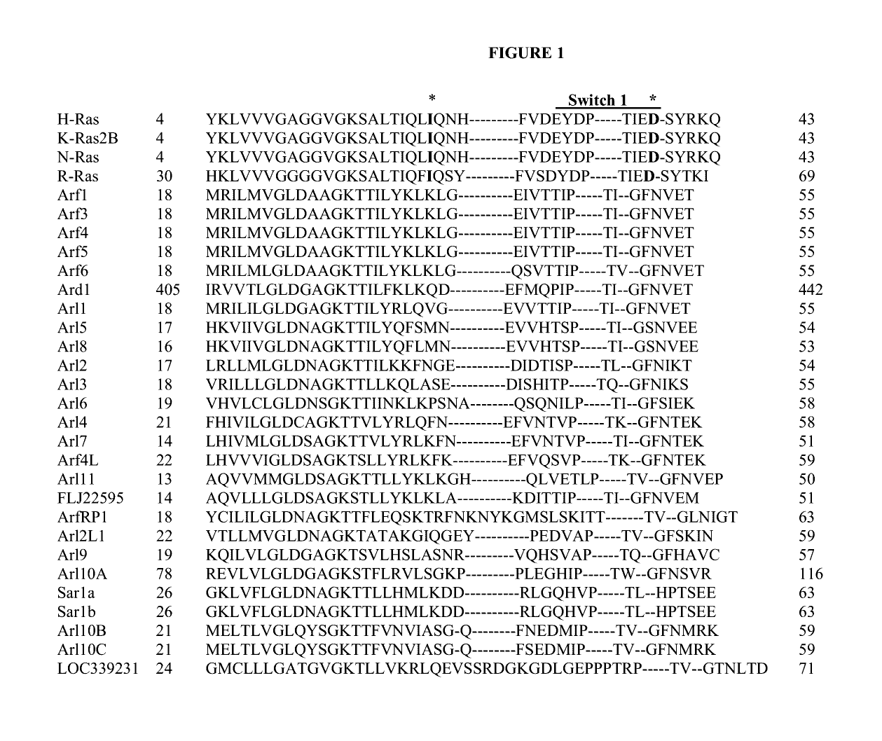

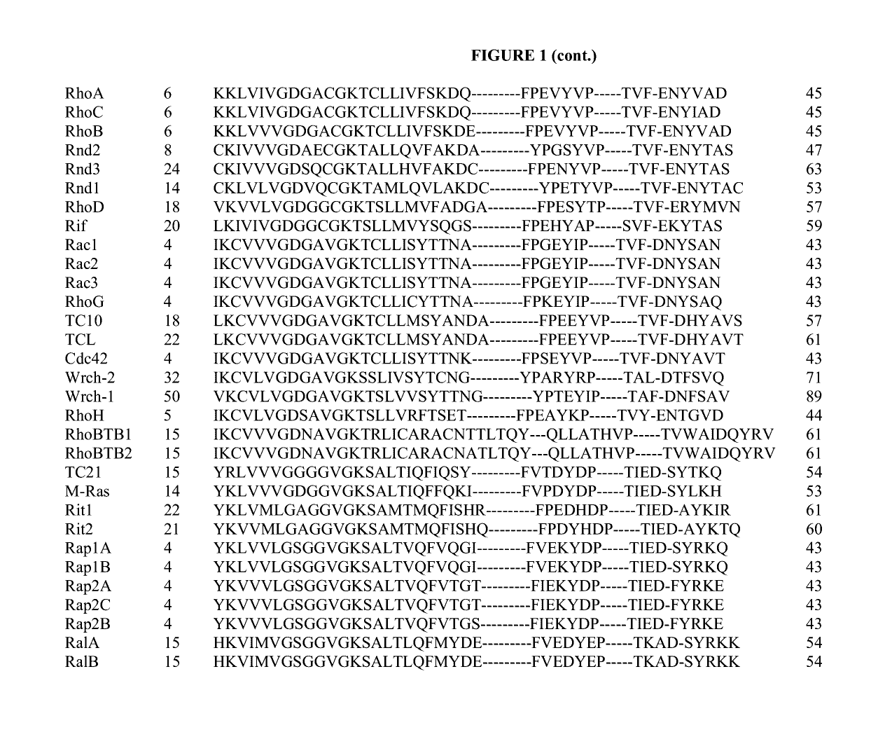

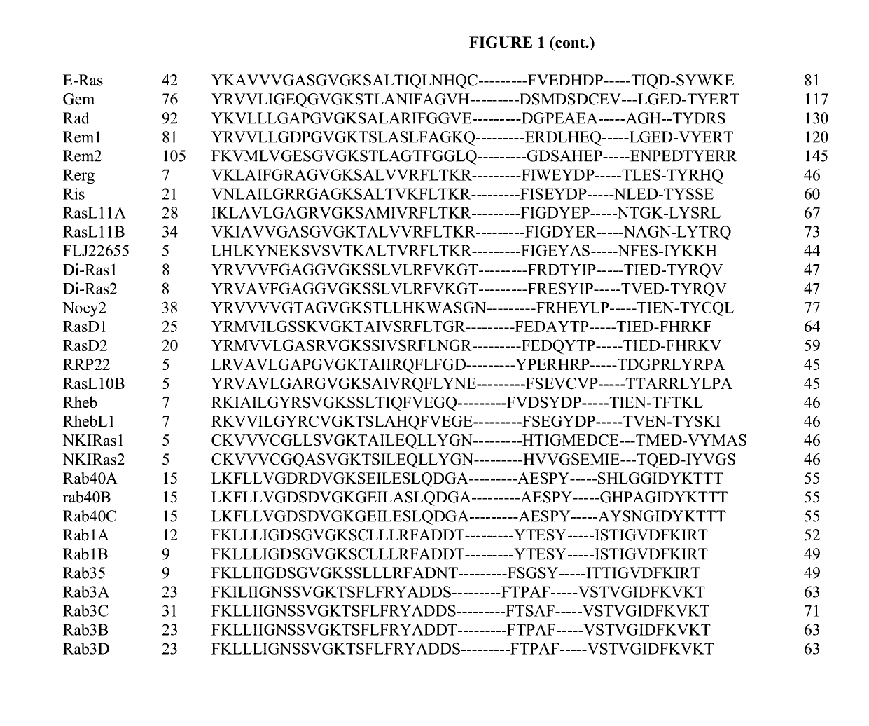

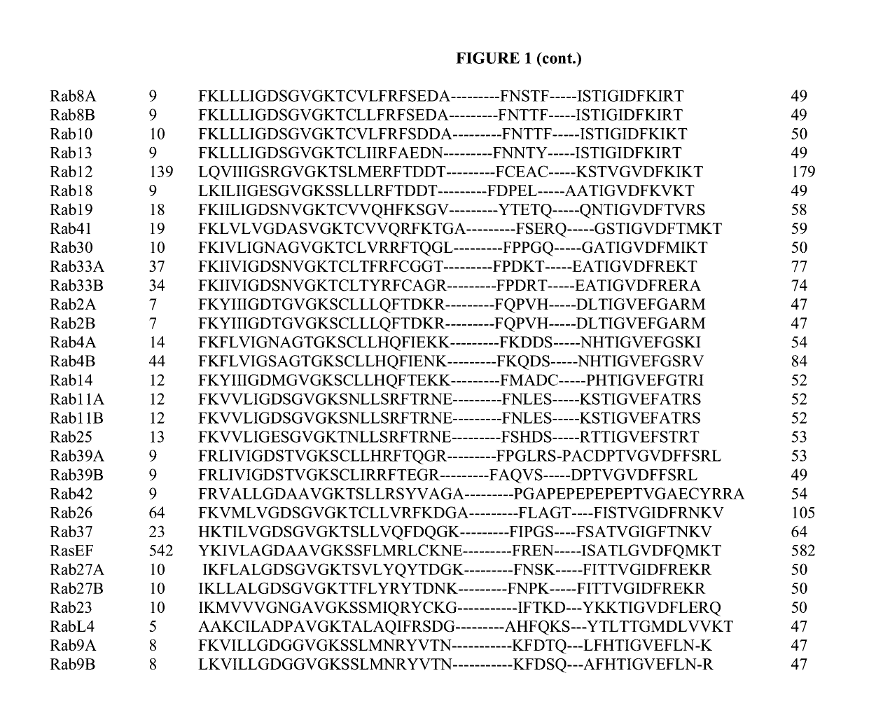

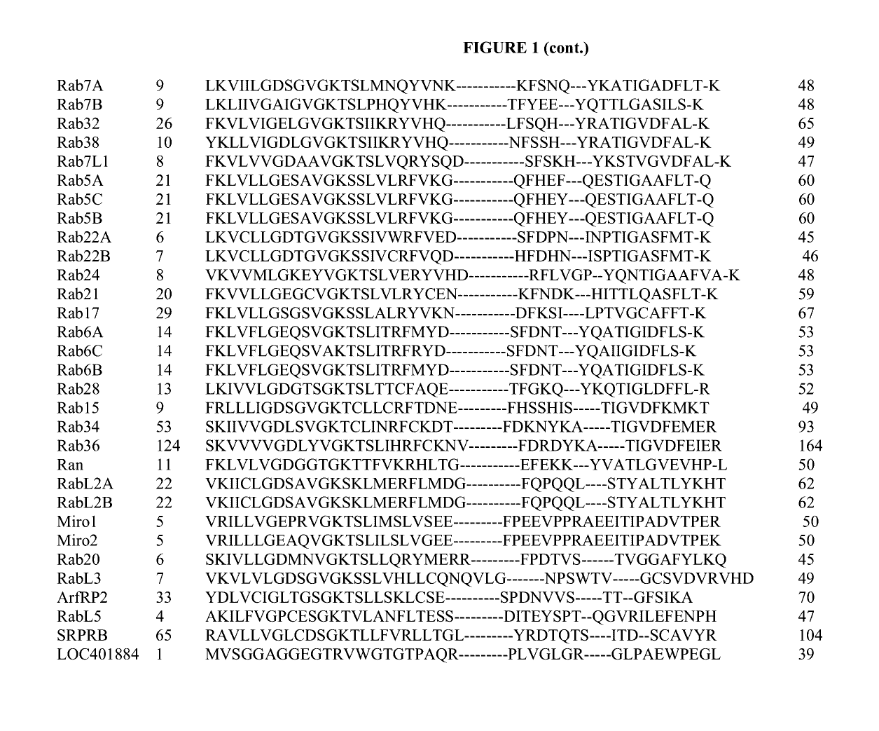

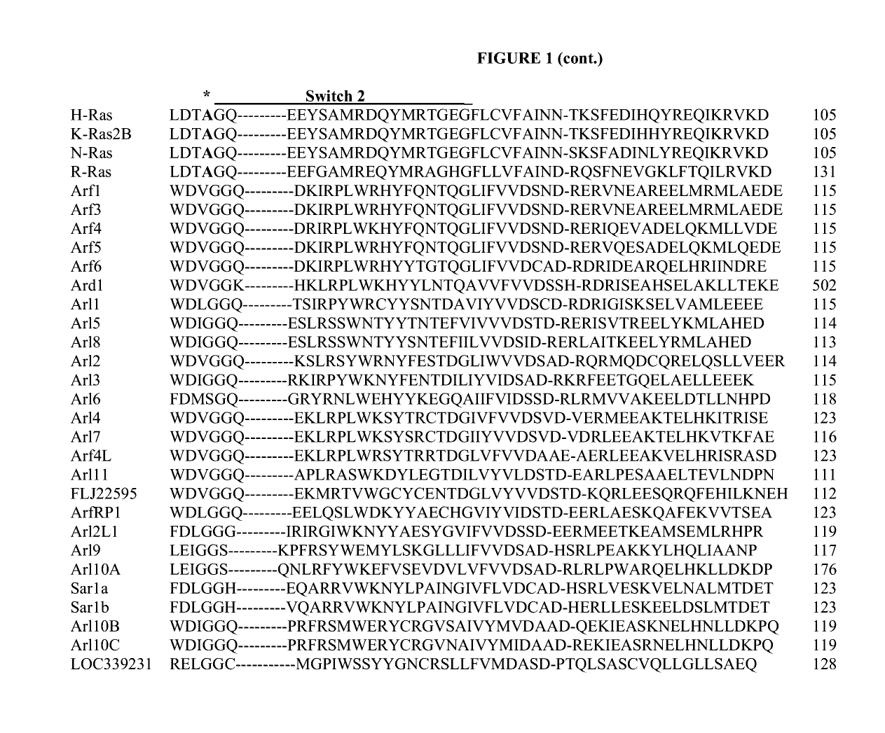

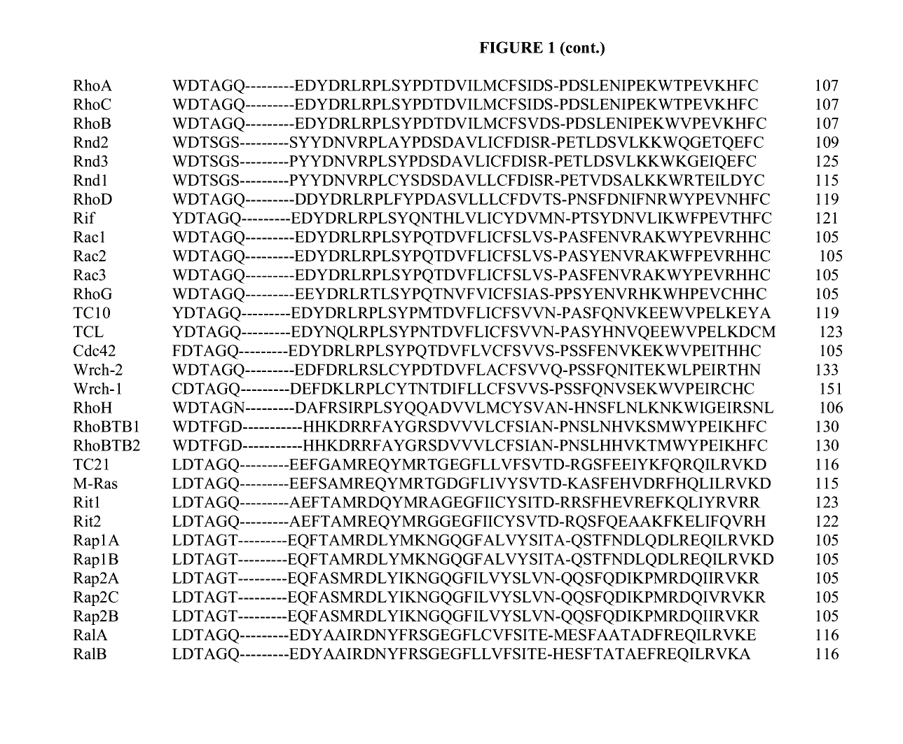

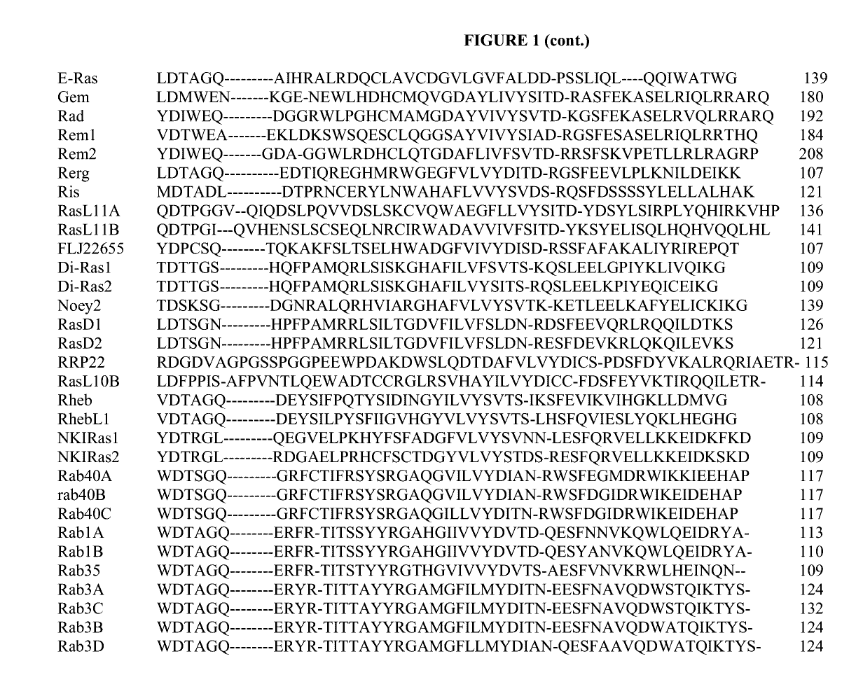

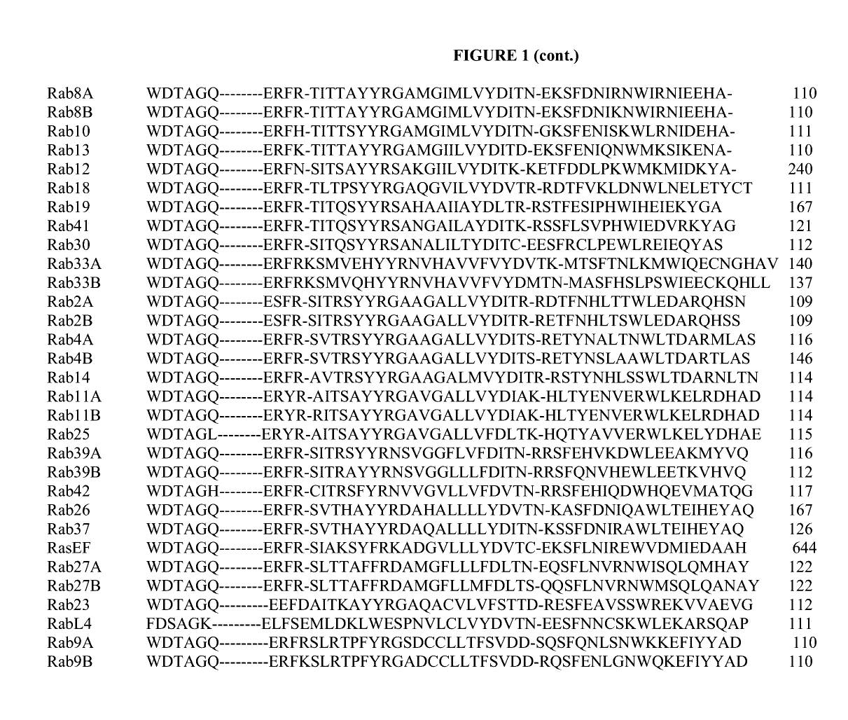

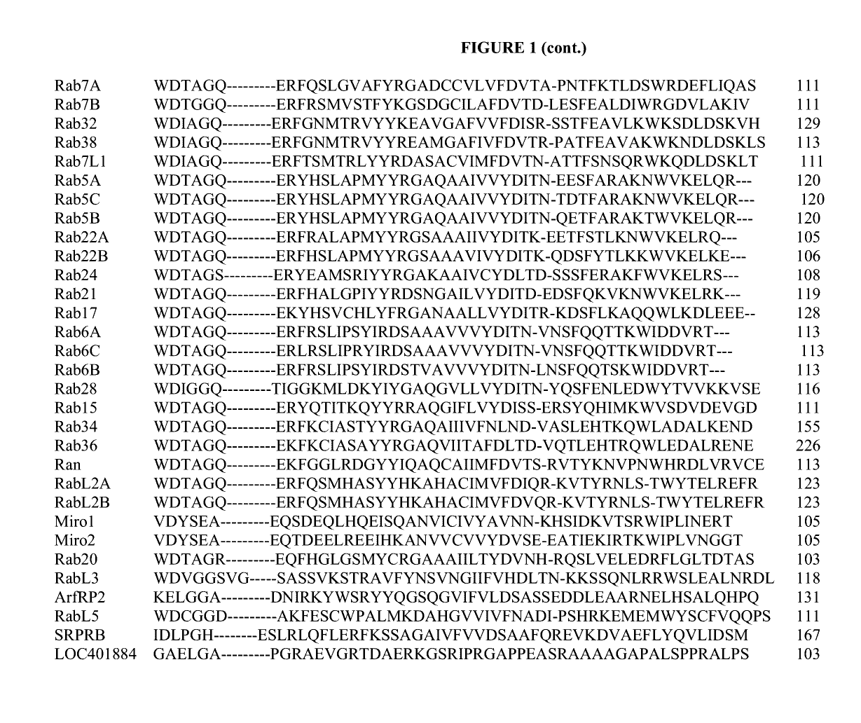

The ˜20 kDa core G domain (corresponding to KRAS residues 4-166) is conserved among most Ras superfamily proteins. This domain is comprised of five conserved guanine nucleotide consensus sequence elements, including the switch 1 (KRAS residues 30-38) and switch 2 (KRAS residues 59-76) regions. Alignment analysis of Ras superfamily proteins reveals high conservation of the G domain region and, notably residues D38, Y21 and A59 of KRAS (FIG. 1, asterisks denoting conserved binding sites D38, A59 and I21).

### Example 57: Biochemical Evaluation of Two-Site Compounds

A set of two-site multivalent compounds is synthesized as described herein with appropriate modifications) and evaluated by HSQC NMR for binding to KRAS^{G12D}. The two-sites are D38 and A59 of KRAS, or sites in the same pocket which are near D38 and A59 of KRAS. The third site is I21 of KRAS, or a site in the same pocket which is near 121 of KRAS. Changes in chemical shift are be observed by dose-dependent shifts by differential scanning fluorimetry. Affinity measurements will be made by microscale thermophoresis (MST).

Affinity to RAS is also be measured by pulldown assays using the RAS binding domain of CRAF. This abrogation of binding between RAS and its effector protein are measured examining RAS-RALGDS interaction. To quantify the binding of the two-site compounds to RAS, MST are performed again using lysine NT-647-labeled, GppNHp-loaded KRAS^{G12D}. To test the whether compounds disclosed herein are selective for the GTP-bound form of RAS, KRAS^{G12D} are loaded with GDP, and measurements for the binding affinity of compounds disclosed herein are done using MST. To evaluate whether binding is occurring in the predicted region of RAS, MST are performed using 136N and D38A mutants. Additional binding studies are performed done using HSQC NMR using GppNHp-loaded KRAS^{G12D} and, as a secondary measure of binding, isothermal titration calorimetry on GppNHp-loaded KRAS^{G12D}. To provide evidence that compounds disclosed herein are selective for RAS GTPases, MST binding measurements are performed on GppNHp-loaded RHEB, RHOA and RALA.

### Example 57: Viability Assay with NCI-H460

### Cell Cultures and Reagents

NCI-H460 (Epithelial lung tissue; carcinoma; large cell lung cancer) were cultured in RPMI Medium 1640 (Invitrogen-22400105) supplemented with 10% fetal bovine serum (FBS; Invitrogen-10099141). All the cell lines were maintained in a humidified incubator at 37° C. with 5% CO2. Cell culture media and supplements were purchased from Invitrogen, and tissue culture flasks were purchased from Corning, 96-well plates were purchased from Greiner. CellTiter-Glo Luminescent Cell Viability Assay kits were purchased from Promega (Promega-G7573), cells counter Vi-Cell was purchased from Beckman, D300e digital dispenser was purchased from Tecan, detection instrument Envision was purchased from PerkinElmer.

Paclitaxel was purchased from SELLECK, test compounds, such as any one of the compounds disclosed herein, attained solubility in DMSO and when diluted into culture media. DMSO, solutions containing the test compounds, and culture media were warmed to 37° C. or room temperature for the solution preparation and dilutions.

### Cytotoxicity Assay

NCI-H460 Cell line were seeded in 96-well plates (1200/well) and allowed to adhere to overnight (100 uL/well), for drug treatments with 2 fold dilution, 9 dose points, triplicates or vehicle control, compound stock solutions were prepared in DMSO and use D300e digital dispenser to add compounds to the wells to give the indicated final drug concentrations. Final DMSO concentration was 0.5%. Cellular ATP concentrations were assessed by using the CellTiter-Glo Cell Viability Assay as per the manufacturer's instructions 72 h after compounds addition. Additional cells lines were assayed with any one of the compounds disclosed herein according to the procedures described for the viability assay with NCI-H460. Table 12 shows the other cells lines that were assayed and Table 13 shows the source and catalog number of the regents and material used.

TABLE 12

| | | | | | Cells |
|---|---|---|---|---|---|
| | Cell | Tumor | Growth | Media | per |
| No. | name | Type | type | type | well |
| 1 | NCI-H460 | Lung cancer | Adherent | RPMI | 1200 |
| | | | | 1640 + 10% | |
| | | | | FBS + 1% PS | |
| 2 | MIA | Pancreatic | Adherent | RPMI | 2000 |
| | PaCa-2 | cancer | | 1640 + 10% | |
| | | (carcinoma) | | FBS + 1% PS | |
| 3 | NCI- | Lung cancer | Adherent | RPMI | 1200 |
| | H2023 | | | 1640 + 10% | |
| | | | | FBS + 2% PS | |
| 4 | U-2 OS | Osteosar- | Adherent | McCoy's | 1500 |
| | | coma | | 5a + 10% | |
| | | | | FBS + 1% PS | |
| 5 | A375 | Malignant | Adherent | DMEM + 10% | 2000 |
| | | melanoma | | FBS + 1% PS | |
| 6 | COLO 205 | Colorectal | Adherent | RPMI | 5000 |
| | | adenocar- | and | 1640 + 10% | |
| | | cinoma | Suspension | FBS + 1% PS | |
| 7 | HT-29 | Colorectal | Adherent | McCoy's | 7000 |
| | | adenocar- | | 5a + 10% | |
| | | cinoma | | FBS + 1% PS | |
| 8 | SK-MEL- | Malignant | Adherent | EMEM + 10% | 4000 |
| | 28 | melanoma | | FBS + 1% PS | |
| 9 | Kasumi-1 | Acute | Suspension | RPMI | 8000 |
| | | myeloblastic | | 1640 + 20% | |
| | | leukemia | | FBS + 1% PS | |

TABLE 13

| Reagent and Materials Name | Source | Catalogue |
|---|---|---|
| RPMI1640 | Invitrogen | 22400105 |
| McCoy's 5A Medium | ATCC | 30-2007 |
| DMEM | Invitrogen | 11995073 |
| EMEM | ATCC | 30-2003 |
| Fetal bovine serum(FBS) | Invitrogen | 10099141 |
| 100X Penicillin-Streptomycin | Hyclone | SV30010 |
| 0.25% Trypsin-EDTA (T-E) | Invitrogen | 25200056 |
| Dulbecco's PBS | Hyclone | SH30028.02B |
| Paclitaxel | SELLECK | S1150 |
| DMSO | Sigma | F472301 |
| 96 Well Microplates | Greiner | 655090 |
| 75 cm² Rectangular Canted Neck | Corning | 430641 |
| Cell Culture Flask with Vent Cap | | |
| CellTiter-Glo | Promega | G7573 |
| EnVision | PerkinElmer | 2104 |
| Vi-cell | Beckman | Vi-cell XR |
| Incubator | Thermo | 371 |
| D300e digital dispenser | Tecan | / |
| T8 Cassette | Tecan | 30064845 |
| D4 Cassette | Tecan | 30084529 |

The results of the viability assay with NCI-H460 cell line are shown below in Table 14.

TABLE 14

| Compound Ref. No. | Mean EC₅₀ |
|---|---|
| Compound C1; HCl | A |
| Compound C2; HCl | A |
| Compound C3; HCl | A |
| Compound C4; HCl | A |
| Compound C5; HCl | A |
| Compound C6; HCl | A |
| Compound C7; Formic acid | A |
| Compound C8; HCl | A |
| Compound C9; HCl | A |
| Compound C10; Formic acid | A |
| Compound C11; HCl | A |
| Compound C12; HCl | A |
| Compound C13; HCl | A |
| Compound C14; HCl | A |
| Compound C15; HCl | A |
| Compound C16; HCl | D |
| Compound C17; HCl | A |
| Compound C18; HCl | A |
| Compound C19; HCl | A |
| Compound C20 | A |
| Compound C21; Formic acid | A |
| Compound C22; Formic acid | A |
| Compound C23; Formic acid | A |
| Compound C24; HCl | A |
| Compound C25; HCl | C |
| Compound C26; HCl | A |
| Compound C27; HCl | D |
| Compound C28 | B |
| Compound C29; HCl | C |
| Compound C30; HCl | A |
| Compound C31; HCl | A |
| Compound C32; HCl | A |
| Compound C33; HCl | A |
| Compound C34; HCl | C |
| Compound C35; HCl | A |
| Compound C36; HCl | D |
| Compound C37; HCl | A |
| Compound C38; HCl | A |
| Compound C39; HCl | A |
| Compound C40; HCl | A |
| Compound C41; Trifluoroacetic acid | A |
| Compound C42; HCl | A |
| Compound C43; HCl | A |
| Compound C44; HCl | A |
| Compound C45; HCl | A |
| Compound C46; HCl | A |
| Compound C47; Formic acid | A |
| Compound C48; HCl | D |
| Compound C49; HCl | B |
| Compound C50; HCl | A |
| Compound C51; HCl | A |
| Compound C52; HCl | A |
| Compound C53; HCl | A |
| Compound C54; HCl | A |
| Compound C55; HCl | A |
| Compound C56; HCl | A |
| Compound C57; HCl | D |
| Compound C58; Formic acid | A |
| Compound C59; Formic acid | A |
| Compound C60; Formic acid | A |
| Compound C61; HCl | A |
| Compound C62; HCl | A |
| Compound C63; HCl | A |
| Compound C64; HCl | A |
| Compound C65; HCl | A |
| Compound C66; HCl | A |
| Compound C67; Formic acid | A |
| Compound C68; Formic acid | A |
| Compound C69; HCl | A |
| Compound C70; HCl | A |
| Compound C71; HCl | A |
| Compound C72; HCl | A |
| Compound C73; Formic acid | A |
| Compound C74; HCl | A |
| Compound C75; HCl | A |
| Compound C76; HCl | B |
| Compound C77; HCl | A |
| Compound C78; HCl | A |
| Compound C79; Formic acid | A |
| Compound C80; HCl | A |
| Compound C81; HCl | A |
| Compound C82; HCl | A |
| Compound C85; HCl | A |
| Compound C86; Formic acid | A |
| Compound C87; Formic acid | A |
| Compound C88; Formic acid | D |
| Compound C89; Formic acid | B |
| Compound C90; HCl | A |
| Compound C90; Formic acid | A |
| Compound C114; HCl | A |
| Compound C128; HCl | A |
| Compound C129; HCl | A |
| Compound C130; HCl | A |
| Compound C131, HCl | A |
| Compound C132; Formic acid | A |
| Compound C133; HCl | A |
| Compound C134; HCl | A |
| Compound C135; HCl | A |
| Compound C136; HCl | A |
| Compound C137; HCl | A |
| Compound C138; HCl | A |
| Compound C139; HCl | A |
| Compound C140; HCl | A |
| Compound C141; HCl | A |
| Compound C142; Formic Acid | A |
| Compound C143; HCl | B |
| Compound C144; HCl | A |
| Compound C145; HCl | A |
| Compound C146; HCl | A |
| Compound C147; HCl | A |
| Compound 148; HCl | A |
| Compound C149; Formic Acid | A |
| Compound C150; Formic acid | A |
| Compound C151; HCl | A |
| Compound C152; HCl | A |
| Compound C153; Formic Acid | A |
| Compound C154; HCl | A |
| Compound C155; HCl | A |
| Compound C156; HCl | A |
| Compound C157; HCl | A |
| Compound C158; HCl | A |
| Compound C159; HCl | A |
| Compound C160; HCl | A |
| Compound C161; HCl | A |
| Compound C162; HCl | A |
| Compound C163; HCl | A |
| Compound C164; HCl | A |
| Compound C165; HCl | A |
| Compound C166; Formic Acid | A |
| Compound C167; HCl | A |
| Compound C168; HCl | A |
| Compound C169; HCl | A |
| Compound C170; HCl | A |
| Compound C171; HCl | A |
| Compound C172; HCl | A |
| Compound C173; HCl | A |
| Compound C174; HCl | A |
| Compound C175; Formic Acid | A |
| Compound C176; HCl | A |
| Compound C177; HCl | A |
| Compound C178; HCl | A |
| Compound C178; HCl | A |
| Compound C179; HCl | A |
| Compound C180; HCl | A |
| Compound C181; HCl | A |
| Compound C182; HCl | A |
| Compound C183; HCl | A |
| Compound C184; HCl | B |
| Compound C185; HCl | B |
| Compound C186; HCl | B |
| Compound C187; HCl | A |
| Compound C188; HCl | B |
| Compound C189; HCl | B |
| Compound C190; HCl | B |
| Compound C191; HCl | B |
| Compound C192; HCl | B |
| Compound C193; HCl | A |
| Compound C194; HCl | B |
| Compound C195; HCl | B |
| Compound C196; HCl | A |
| Compound C197; HCl | A |
| Compound C198; HCl | A |
| Compound C199; HCl | B |
| Compound C200; HCl | B |
| Compound C201; HCl | A |
| Compound C202; Formic Acid | A |
| Compound C203; HCl | A |
| Compound C204; HCl | A |
| Compound C205; Formic Acid | B |
| Compound C206; HCl | A |
| Compound C207; HCl | B |
| Compound C208; Formic Acid | A |
| Compound C209; HCl | A |
| Compound C210; HCl | A |
| Compound C212; HCl | A |
| Compound C213; Formic Acid | A |
| Compound C214; Formic Acid | A |
| Compound C215; HCl | A |
| Compound C216; HCl | A |
| Compound C217; HCl | A |
| Compound C218; Formic Acid | A |
| Compound C219; HCl | A |
| Compound C220; Formic Acid | B |
| Compound C221; HCl | A |
| Compound C222; HCl | A |
| Compound C223; HCl | A |
| Compound C224; Formic Acid | A |
| Compound C225; HCl | A |
| Compound C226; HCl | A |
| Compound C227; HCl | A |
| Compound C228; HCl | A |
| Compound C229; Formic Acid | A |
| Compound C230; HCl | A |
| Compound C231; HCl | A |
| Compound C232; Formic Acid | A |
| Compound C233; HCl | A |
| Compound C234; Formic Acid | A |
| Compound C235; Formic Acid | A |
| Compound C236; HCl | A |
| Compound C237; HCl | A |
| Compound C238; HCl | A |
| Compound C239; Formic Acid | A |
| Compound C240; Formic Acid | A |
| Compound C241; HCl | A |
| Compound C242; HCl | B |
| Compound C243; HCl | B |
| Compound C245; Formic Acid | A |
| Compound C246; Formic Acid | B |
| Compound C247; HCl | A |
| Compound C248; HCl | B |
| Compound C249; Formic Acid | A |
| Compound C250; HCl | A |
| Compound C251; HCl | A |
| Compound C252; HCl | A |
| Compound C253; HCl | A |
| Compound C254; HCl | A |
| Compound C255; HCl | B |
| Compound C256; HCl | B |
| Compound C257; HCl | A |
| Compound C258; Formic Acid | B |
| Compound C259; HCl | B |
| Compound C260; HCl | B |
| Compound C261; HCl | A |
| Compound C262; HCl | A |
| Compound C263; Formic Acid | A |
| Compound C264; HCl | B |
| Compound C265; HCl | A |
| Compound C266; HCl | A |
| Compound C267; HCl | A |
| Compound C268; HCl | B |
| Compound C269; Formic Acid | A |
| Compound C270; HCl | A |
| Compound C271; HCl | A |
| Compound C272; HCl | A |
| Compound C273; HCl | A |
| Compound C274; HCl | A |
| Compound C275; HCl | A |
| Compound C276; HCl | A |
| Compound C277; HCl | A |
| Compound C278; HCl | A |
| Compound C279; Formic Acid | A |
| Compound C280; HCl | A |
| Compound C281; HCl | A |
| Compound C282; HCl | A |
| Compound C283; HCl | A |
| Compound C284; HCl | A |
| Compound C285; HCl | A |
| Compound C286; HCl | B |
| Compound C287; HCl | A |
| Compound C288; HCl | A |
| Compound C289; HCl | B |
| Compound C290; HCl | B |
| Compound C291; HCl | A |
| Compound C292; Formic Acid | A |
| Compound C293; HCl | A |
| Compound C294; HCl | A |
| Compound C295; HCl | A |
| Compound C296; HCl | A |
| Compound C297; HCl | A |
| Compound C298; HCl | A |
| Compound C302; HCl | A |
| Compound C303; HCl | A |
| Compound C304; HCl | A |
| Compound C305; HCl | A |
| Compound C306; HCl | A |
| Compound C307; HCl | A |
| Compound C308; HCl | A |
| Compound C309; HCl | A |

Note:
Biochemical assay Mean EC₅₀ data are designated within the following ranges: A: ≦5 μM; B: >5 μM to ≦10 μM; C: >10 μM to ≦30 μM; and D: >30 μM.

### Example 58: Viability Assay with MIA PaCa-2

A viability assay with MIA PaCa-2 (Epithelial pancreas tissue; carcinoma) was performed with compounds listed in Table 15, according to the procedures described in Example 57. The results of viability assay with MIA PaCa-2 are shown in below in Table 15.

TABLE 15

| Compound Ref. No. | Mean EC₅₀ |
|---|---|
| Compound C1; HCl | A |
| Compound C2; HCl | A |
| Compound C3; HCl | A |
| Compound C4; HCl | A |
| Compound C5; HCl | A |
| Compound C6; HCl | A |
| Compound C7; Formic acid | A |
| Compound C8; HCl | A |
| Compound C9; HCl | A |
| Compound C10; Formic acid | A |
| Compound C11; HCl | A |
| Compound C12; HCl | A |
| Compound C13; HCl | A |
| Compound C14; HCl | A |
| Compound C15; HCl | A |
| Compound C16; HCl | D |
| Compound C17; HCl | A |
| Compound C18; HCl | A |
| Compound C19; HCl | A |
| Compound C20 | A |
| Compound C21; Formic acid | A |
| Compound C22; Formic acid | A |
| Compound C23; Formic acid | A |
| Compound C24; HCl | A |
| Compound C25; HCl | C |
| Compound C26; HCl | A |
| Compound C27; HCl | C |
| Compound C28 | A |
| Compound C29; HCl | D |
| Compound C30; HCl | A |
| Compound C31; HCl | A |
| Compound C32; HCl | A |
| Compound C33; HCl | A |
| Compound C34; HCl | C |
| Compound C35; HCl | A |
| Compound C36; HCl | C |
| Compound C37; HCl | B |
| Compound C38; HCl | A |
| Compound C39; HCl | A |
| Compound C40; HCl | A |
| Compound C41; Trifluoroacetic acid | A |
| Compound C42; HCl | A |
| Compound C43; HCl | A |
| Compound C44; HCl | A |
| Compound C45; HCl | A |
| Compound C46; HCl | A |
| Compound C47; Formic acid | A |
| Compound C48; HCl | D |
| Compound C49; HCl | B |
| Compound C50; HCl | A |
| Compound C51; HCl | A |
| Compound C52; HCl | A |
| Compound C53; HCl | A |
| Compound C54; HCl | A |
| Compound C55; HCl | A |
| Compound C56; HCl | A |
| Compound C57; HCl | D |
| Compound C58; Formic acid | A |
| Compound C59; Formic acid | A |
| Compound C60; Formic acid | A |
| Compound C61; HCl | A |
| Compound C62; HCl | A |
| Compound C63; HCl | A |
| Compound C64; HCl | A |
| Compound C65; HCl | A |
| Compound C66; HCl | A |
| Compound C67; Formic acid | A |
| Compound C68; Formic acid | A |
| Compound C69; HCl | A |
| Compound C70; HCl | A |
| Compound C71; HCl | A |
| Compound C72; HCl | A |
| Compound C73; Formic acid | A |
| Compound C74; HCl | A |
| Compound C75; HCl | A |
| Compound C76; HCl | A |
| Compound C77; HCl | A |
| Compound C78; HCl | A |
| Compound C79; Formic acid | A |
| Compound C80; HCl | A |
| Compound C81; HCl | A |
| Compound C82; HCl | A |
| Compound C85; HCl | A |
| Compound C86; Formic acid | A |
| Compound C87; Formic acid | A |
| Compound C88; Formic acid | D |
| Compound C89; Formic acid | B |
| Compound C90; HCl | A |
| Compound C90; Formic acid | A |
| Compound C114; HCl | A |
| Compound C128; HCl | A |
| Compound C129; HCl | A |
| Compound C130; HCl | A |
| Compound C131, HCl | A |
| Compound C132; Formic acid | A |
| Compound C133; HCl | A |
| Compound C134; HCl | A |
| Compound C135; HCl | A |
| Compound C136; HCl | A |
| Compound C137; HCl | A |
| Compound C138; HCl | A |
| Compound C139; HCl | A |
| Compound C140; HCl | A |
| Compound C141; HCl | A |
| Compound C142; Formic Acid | B |
| Compound C143; HCl | B |
| Compound C144; HCl | A |
| Compound C145; HCl | A |
| Compound C146; HCl | A |
| Compound C147; HCl | A |
| Compound C148; HCl | A |
| Compound C149; Formic Acid | A |
| Compound C150; Formic acid | A |
| Compound C151; HCl | A |
| Compound C152; HCl | A |
| Compound C153; Formic Acid | A |
| Compound C154; HCl | A |
| Compound C155; HCl | A |
| Compound C156; HCl | A |
| Compound C157; HCl | A |
| Compound C158; HCl | A |
| Compound C159; HCl | A |
| Compound C160; HCl | A |
| Compound C161; HCl | A |
| Compound C162; HCl | A |
| Compound C163; HCl | A |
| Compound C164; HCl | A |
| Compound C165; HCl | A |
| Compound C166; Formic Acid | A |
| Compound C167; HCl | A |
| Compound C168; HCl | A |
| Compound C169; HCl | A |
| Compound C170; HCl | A |
| Compound C171; HCl | A |
| Compound C172; HCl | A |
| Compound C173; HCl | A |
| Compound C174; HCl | A |
| Compound C175; Formic Acid | B |
| Compound C176; HCl | A |
| Compound C177; HCl | A |
| Compound C178; HCl | A |
| Compound C178; HCl | A |
| Compound C179; HCl | A |
| Compound C180; HCl | A |
| Compound C181; HCl | A |
| Compound C182; HCl | A |
| Compound C183; HCl | A |
| Compound C184; HCl | B |
| Compound C185; HCl | B |
| Compound C186; HCl | B |
| Compound C187; HCl | A |
| Compound C188; HCl | B |
| Compound C189; HCl | B |
| Compound C190; HCl | B |
| Compound C191; HCl | B |
| Compound C192; HCl | B |
| Compound C193; HCl | A |
| Compound C194; HCl | B |
| Compound C195; HCl | B |
| Compound C196; HCl | A |
| Compound C197; HCl | A |
| Compound C198; HCl | A |
| Compound C199; HCl | B |
| Compound C200; HCl | B |
| Compound C201; HCl | A |
| Compound C202; Formic Acid | B |
| Compound C203; HCl | A |
| Compound C204; HCl | A |
| Compound C205; Formic Acid | B |
| Compound C206; HCl | A |
| Compound C207; HCl | B |
| Compound C208; Formic Acid | B |
| Compound C209; HCl | A |
| Compound C210; HCl | A |
| Compound C212; HCl | A |
| Compound C213; Formic Acid | A |
| Compound C214; Formic Acid | A |
| Compound C215; HCl | A |
| Compound C216; HCl | A |
| Compound C217; HCl | A |
| Compound C218; Formic Acid | A |
| Compound C219; HCl | A |
| Compound C220; Formic Acid | B |
| Compound C221; HCl | A |
| Compound C222; HCl | A |
| Compound C223; HCl | A |
| Compound C224; Formic Acid | A |
| Compound C225; HCl | A |
| Compound C226; HCl | A |
| Compound C227; HCl | A |
| Compound C228; HCl | A |
| Compound C229; Formic Acid | A |
| Compound C230; HCl | A |
| Compound C231; HCl | A |
| Compound C232; Formic Acid | A |
| Compound C233; HCl | B |
| Compound C234; Formic Acid | A |
| Compound C235; Formic Acid | A |
| Compound C236; HCl | A |
| Compound C237; HCl | A |
| Compound C238; HCl | A |
| Compound C239; Formic Acid | A |
| Compound C240; Formic Acid | A |
| Compound C241; HCl | A |
| Compound C242; HCl | B |
| Compound C243; HCl | B |
| Compound C245; Formic Acid | A |
| Compound C246; Formic Acid | B |
| Compound C247; HCl | A |
| Compound C248; HCl | B |
| Compound C249; Formic Acid | A |
| Compound C250; HCl | A |
| Compound C251; HCl | A |
| Compound C252; HCl | A |
| Compound C253; HCl | A |
| Compound C254; HCl | A |
| Compound C255; HCl | A |
| Compound C256; HCl | B |
| Compound C257; HCl | A |
| Compound C258; Formic Acid | B |
| Compound C259; HCl | B |
| Compound C260; HCl | A |
| Compound C261; HCl | A |
| Compound C262; HCl | A |
| Compound C263; Formic Acid | A |
| Compound C264; HCl | B |
| Compound C265; HCl | A |
| Compound C266; HCl | A |
| Compound C267; HCl | A |
| Compound C268; HCl | B |
| Compound C269; Formic Acid | A |
| Compound C270; HCl | A |
| Compound C271; HCl | A |
| Compound C272; HCl | B |
| Compound C273; HCl | A |
| Compound C274; HCl | A |
| Compound C275; HCl | A |
| Compound C276; HCl | B |
| Compound C277; HCl | A |
| Compound C278; HCl | A |
| Compound C279; Formic Acid | A |
| Compound C280; HCl | A |
| Compound C281; HCl | A |
| Compound C282; HCl | A |
| Compound C283; HCl | A |
| Compound C284; HCl | A |
| Compound C285; HCl | A |
| Compound C286; HCl | B |
| Compound C287; HCl | A |
| Compound C288; HCl | A |
| Compound C289; HCl | B |
| Compound C290; HCl | B |
| Compound C291; HCl | A |
| Compound C292; Formic Acid | A |
| Compound C293; HCl | A |
| Compound C294; HCl | A |
| Compound C295; HCl | A |
| Compound C296; HCl | A |
| Compound C297; HCl | A |
| Compound C298; HCl | B |
| Compound C302; HCl | A |
| Compound C303; HCl | A |
| Compound C304; HCl | A |
| Compound C305; HCl | A |
| Compound C306; HCl | A |
| Compound C307; HCl | A |
| Compound C308; HCl | A |
| Compound C309; HCl | A |

Note:
Biochemical assay Mean EC₅₀ data are designated within the following ranges: A: ≦5 μM; B: >5 μM to ≦10 μM; C: >10 μM to ≦30 μM; and D: >30 μM.

### Example 59: Viability Assay with NCI-H2023

A viability assay with NCI-H2023 (stage 3A, adenocarcinoma; non-small cell lung cancer) was performed with compounds listed in Table 16 according to the procedures described in Example 57. The results of viability assay with NCI-H2023 are shown in below in Table 16.

TABLE 16

| Compound Ref. No. | Mean EC₅₀ |
|---|---|
| Compound C13; HCl | A |
| Compound C14; HCl | A |
| Compound C15; HCl | A |
| Compound C16; HCl | D |
| Compound C17; HCl | A |
| Compound C18; HCl | A |
| Compound C19; HCl | A |
| Compound C20 | A |
| Compound C21; Formic acid | A |
| Compound C22; Formic acid | A |
| Compound C23; Formic acid | A |
| Compound C24; HCl | A |
| Compound C25; HCl | C |
| Compound C26; HCl | A |
| Compound C27; HCl | C |
| Compound C28 | B |
| Compound C29; HCl | C |
| Compound C30; HCl | A |
| Compound C31; HCl | A |
| Compound C32; HCl | A |
| Compound C33; HCl | A |
| Compound C34; HCl | C |
| Compound C35; HCl | A |
| Compound C36; HCl | D |
| Compound C37; HCl | B |
| Compound C38; HCl | A |
| Compound C39; HCl | A |
| Compound C40; HCl | A |
| Compound C41; Trifluoroacetic acid | A |
| Compound C42; HCl | A |
| Compound C43; HCl | A |
| Compound C44; HCl | A |
| Compound C45; HCl | A |
| Compound C46; HCl | A |
| Compound C47; Formic acid | A |
| Compound C48; HCl | D |
| Compound C49; HCl | B |
| Compound C50; HCl | A |
| Compound C51; HCl | A |
| Compound C52; HCl | A |
| Compound C53; HCl | A |
| Compound C54; HCl | A |
| Compound C55; HCl | A |
| Compound C56; HCl | A |

Note:
Biochemical assay Mean EC₅₀ data are designated within the following ranges: A: ≦5 μM; B: >5 μM to ≦10 μM; C: >10 μM to ≦30 μM; D: >30 μM

### Example 60: Viability Assay with U2OS

The viability assay with U2OS (epithelial bone tissue; osteosarcoma) was performed with compounds listed in Table 17 according to the procedures described in Example 57. The results of viability assay with U2OS are shown in below in Table 17.

TABLE 17

| Compound Ref. No. | Mean EC₅₀ |
|---|---|
| Compound C1; HCl | A |
| Compound C2; HCl | A |
| Compound C3; HCl | A |
| Compound C4; HCl | A |
| Compound C5; HCl | A |
| Compound C6; HCl | A |
| Compound C7; Formic acid | A |
| Compound C8; HCl | A |
| Compound C9; HCl | A |
| Compound C10; Formic acid | A |
| Compound C11; HCl | A |
| Compound C12; HCl | A |
| Compound C22; Formic acid | A |
| Compound C43; HCl | A |
| Compound C155; HCl | A |
| Compound C156; HCl | A |
| Compound C157; HCl | A |
| Compound C158; HCl | A |
| Compound C159; HCl | A |
| Compound C160; HCl | A |
| Compound C162; HCl | A |
| Compound C163; HCl | A |
| Compound C164; HCl | A |
| Compound C165; HCl | A |
| Compound C166; Formic Acid | A |
| Compound C167; HCl | A |
| Compound C168; HCl | A |
| Compound C169; HCl | A |
| Compound C172; HCl | A |
| Compound C173; HCl | A |

Note:
Biochemical assay Mean EC₅₀ data are designated within the following ranges: A: ≦5 μM; B: >5 μM to ≦10 μM; C: >10 μM to ≦30 μM; D: >30 μM

### Example 61: Viability Assay with HT-29

A viability assay with HT-29 (Colorectal adenocarcinoma) was performed with compounds listed in Table 18 according to the procedures described in Example 57. The results of viability assay with HT-29 are shown in below in Table 18.

TABLE 18

| | |
|---|---|
| Compound C128; HCl | A |
| Compound C129; HCl | A |
| Compound C130; HCl | A |
| Compound C131; HCl | A |
| Compound C132; Formic acid | A |
| Compound C133; HCl | A |
| Compound C134; HCl | A |
| Compound C135; HCl | A |
| Compound C136; HCl | A |
| Compound C137; HCl | A |
| Compound C138; HCl | A |
| Compound C139; HCl | A |
| Compound C140; HCl | A |
| Compound C141; HCl | A |
| Compound C142; Formic Acid | B |
| Compound C143; HCl | B |
| Compound C144; HCl | A |
| Compound C145; HCl | A |
| Compound C146; HCl | A |
| Compound C147; HCl | A |
| Compound C148; HCl | A |
| Compound C149; Formic Acid | A |
| Compound C150; Formic acid | A |
| Compound C151; HCl | A |
| Compound C152; HCl | A |
| Compound C153; Formic Acid | A |
| Compound C154; HCl | A |
| Compound C156; HCl | A |
| Compound C162; HCl | A |
| Compound C166; Formic Acid | A |
| Compound C168; HCl | A |
| Compound C169; HCl | A |
| Compound C170; HCl | A |
| Compound C171; HCl | A |
| Compound C178; HCl | A |
| Compound C179; HCl | A |
| Compound C180; HCl | A |
| Compound C181; HCl | A |
| Compound C182; HCl | A |
| Compound C183; HCl | A |
| Compound C184; HCl | B |
| Compound C185; HCl | B |
| Compound C186; HCl | B |
| Compound C187; HCl | A |
| Compound C188; HCl | B |
| Compound C189; HCl | A |
| Compound C190; HCl | B |
| Compound C191; HCl | B |
| Compound C192; HCl | B |
| Compound C193; HCl | A |
| Compound C194; HCl | B |
| Compound C195; HCl | B |
| Compound C196; HCl | A |
| Compound C197; HCl | A |
| Compound C198; HCl | A |
| Compound C199; HCl | B |
| Compound C200; HCl | B |
| Compound C201; HCl | A |
| Compound C202; Formic Acid | B |
| Compound C203; HCl | A |
| Compound C204; HCl | A |
| Compound C205; Formic Acid | B |
| Compound C206; HCl | A |
| Compound C207; HCl | B |
| Compound C208; Formic Acid | B |
| Compound C209; HCl | A |
| Compound C210; HCl | A |
| Compound C212; HCl | A |
| Compound C213; Formic Acid | A |
| Compound C214; Formic Acid | A |
| Compound C215; HCl | A |
| Compound C216; HCl | A |
| Compound C217; HCl | A |
| Compound C218; Formic Acid | A |
| Compound C219; HCl | A |
| Compound C220; Formic Acid | B |
| Compound C221; HCl | A |
| Compound C222; HCl | A |
| Compound C223; HCl | A |
| Compound C224; Formic Acid | A |
| Compound C225; HCl | A |
| Compound C226; HCl | A |
| Compound C227; HCl | A |
| Compound C228; HCl | A |
| Compound C229; Formic Acid | A |
| Compound C230; HCl | A |
| Compound C231; HCl | A |
| Compound C232; Formic Acid | A |
| Compound C233; HCl | B |
| Compound C234; Formic Acid | A |
| Compound C235; Formic Acid | A |
| Compound C236; HCl | A |
| Compound C237; HCl | A |
| Compound C238; HCl | A |
| Compound C239; Formic Acid | A |
| Compound C240; Formic Acid | A |
| Compound C241; HCl | A |
| Compound C242; HCl | B |
| Compound C243; HCl | B |
| Compound C245; Formic Acid | A |
| Compound C246; Formic Acid | C |
| Compound C247; HCl | A |
| Compound C248; HCl | B |
| Compound C249; Formic Acid | A |
| Compound C250; HCl | A |
| Compound C251; HCl | A |
| Compound C252; HCl | A |
| Compound C253; HCl | A |
| Compound C254; HCl | A |
| Compound C255; HCl | B |
| Compound C256; HCl | B |
| Compound C257; HCl | A |
| Compound C258; Formic Acid | B |
| Compound C259; HCl | B |
| Compound C260; HCl | B |
| Compound C261; HCl | A |
| Compound C262; HCl | A |
| Compound C263; Formic Acid | B |
| Compound C264; HCl | B |
| Compound C265; HCl | A |
| Compound C266; HCl | A |
| Compound C267; HCl | A |
| Compound C268; HCl | A |
| Compound C269; Formic Acid | A |
| Compound C270; HCl | A |
| Compound C271; HCl | A |
| Compound C272; HCl | A |
| Compound C273; HCl | A |
| Compound C274; HCl | A |
| Compound C275; HCl | A |
| Compound C276; HCl | B |
| Compound C277; HCl | A |
| Compound C278; HCl | A |
| Compound C279; Formic Acid | A |
| Compound C280; HCl | A |
| Compound C281; HCl | A |
| Compound C282; HCl | A |
| Compound C283; HCl | A |
| Compound C284; HCl | A |
| Compound C285; HCl | A |
| Compound C286; HCl | B |
| Compound C287; HCl | A |
| Compound C288; HCl | A |
| Compound C289; HCl | B |
| Compound C290; HCl | B |
| Compound C291; HCl | A |
| Compound C292; Formic Acid | B |
| Compound C293; HCl | A |
| Compound C294; HCl | A |
| Compound C295; HCl | A |
| Compound C296; HCl | A |
| Compound C297; HCl | A |
| Compound C298; HCl | A |

Note:
Biochemical assay Mean EC₅₀ data are designated within the following ranges: A: ≦5 μM; B: >5 μM to ≦10 μM; C: >10 μM to ≦30 μM; D: >30 μM.

### Example 62: Inhibition of Ras Signaling

The ability of compounds disclosed herein to disrupt RAS-RAF-MEK-ERK signaling is examined by measuring phosphorylated ERK abundance upon compound treatment, compared to the MEK1/2 inhibitor U0126. To test if compounds disclosed herein are capable of preventing the interaction between RAS and RALGDS (a guanine dissociation stimulator of RALA), a RALA activation assay is performed using RALBP1. To provide further confirmation of direct disruption of RAS-RAF and RAS-PI3K, immunoprecipitation using an HRAS antibody is performed and the resulting western blot is tested for the presences of cRAF and PI3Kgamma.

The consequences of RAS family inhibitors disclosed herein are investigated at the transcriptional level. To determine mRNA expression differences manifest upon RAS activation, BJeLR (HRAS^{G12V}) and BJeHLT (wt HRAS) engineered isogenic fibroblasts that differ only by HRAS^{G12V} overexpression in BJeLR cells will be used. The expression of urokinase-type plasminogen activator (uPA) is associated with invasion, metastasis and angiogenesis via breakdown of various components of the extracellular matrix; uPA overexpression is facilitated by RAS activation through the RAS-RALGDS-RAL pathway. Inhibition of this cascade is tested for by analyzing uPA expression levels, via qPCR, in BJeLR (DMSO treated) versus BJeLR (compound treated at multiple doses) and BJHLT (DMSO treated). In addition, other downstream signaling events include and are not limited to CMYC, MMP, and/or lactate dehydrogenase (LDH) overexpression.

The examples and embodiments described herein are for illustrative purposes only and various modifications or changes suggested to persons skilled in the art are to be included within the spirit and purview of this application and scope of the appended claims.

What is claimed is:

1. A compound of Formula (Ia), or a pharmaceutically acceptable salt, or solvate thereof: 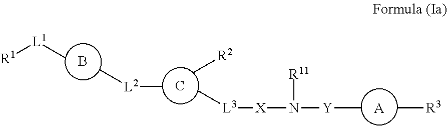
wherein, 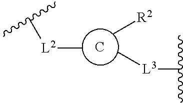
is a bicyclic heteroaryl that is selected from the following structures: 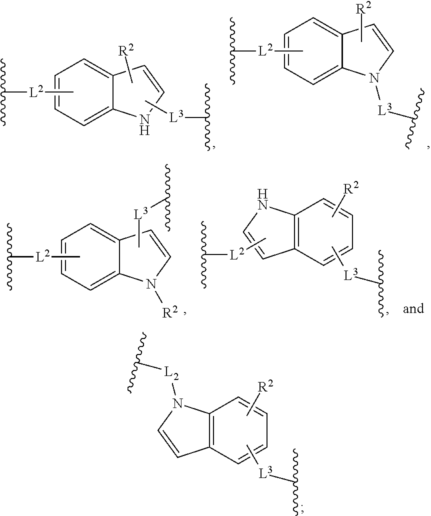
L¹ and L² are each independently an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N with the proviso that Ring B is not: 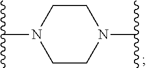 [SMILES: *N1CCN(*)CC1]
wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)— or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Y is —C(R⁷)(R⁸)— or C(═O);
R⁷ and R⁸ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
Ring A is an optionally substituted heterocycloalkyl ring containing at least one N;
wherein if Ring A is substituted, then Ring A is substituted with at least one R^{A};
each R^{A} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
R³ is H, CH₂N(R⁹)(R¹⁰), or N(R⁹)(R¹⁰);
R⁹ and R¹⁰ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁹ and R¹⁰ are taken together with the N atom to which they are attached to form an optionally substituted heterocycloalkyl ring, and
R⁴ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring,
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl).

2. The compound of claim 1, or a pharmaceutically acceptable salt, or solvate thereof, wherein:

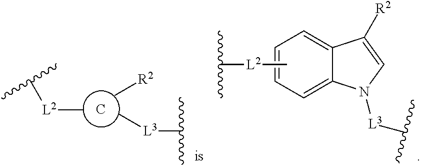

3.-4. (canceled)

5. The compound of claim 1, or a pharmaceutically acceptable salt, or solvate thereof, wherein:

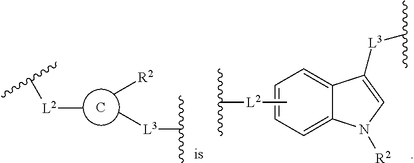

6.-7. (canceled)

8. The compound of claim 1, or a pharmaceutically acceptable salt, or solvate thereof, wherein 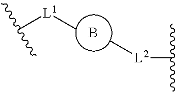
is selected from the following: 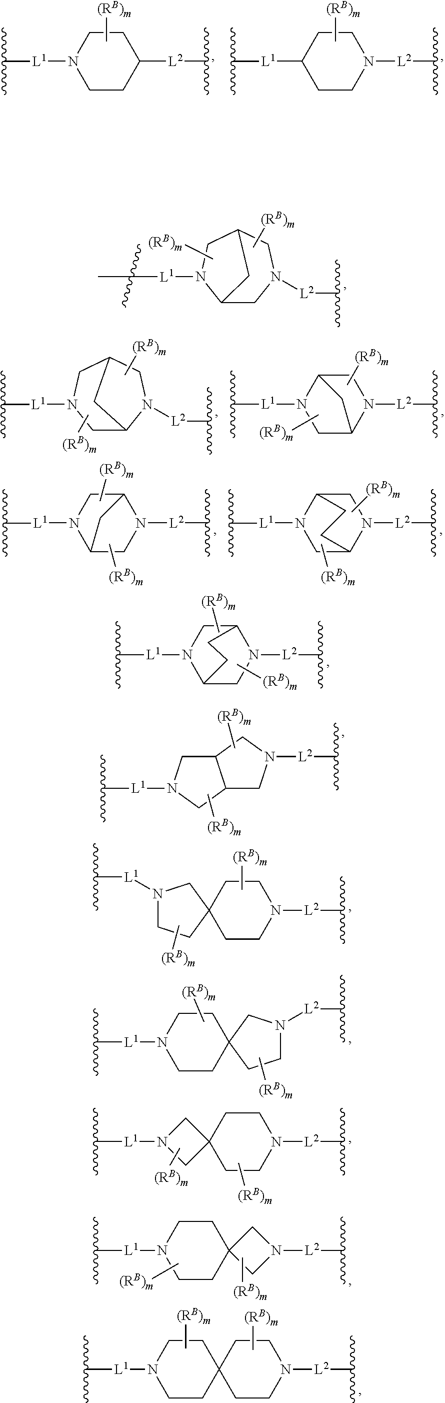 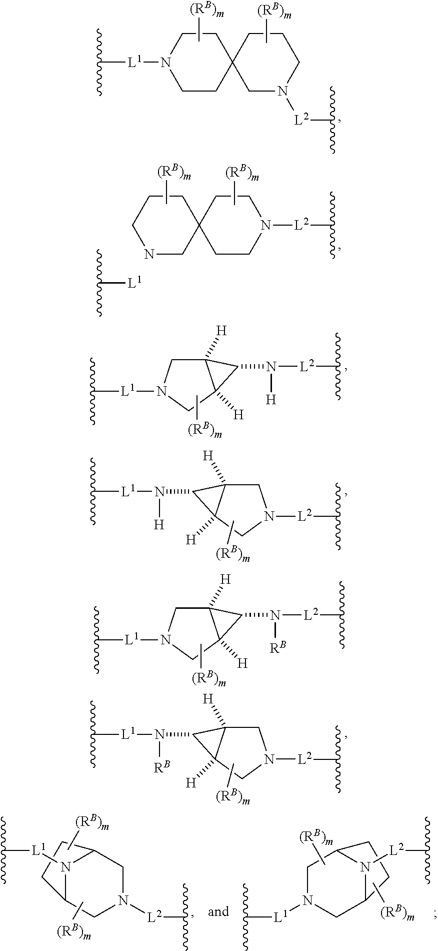
and each m is independently 0, 1, 2, 3, or 4.

9. The compound of claim 1, or a pharmaceutically acceptable salt, or solvate thereof, wherein 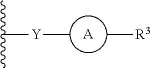
is selected from the following: 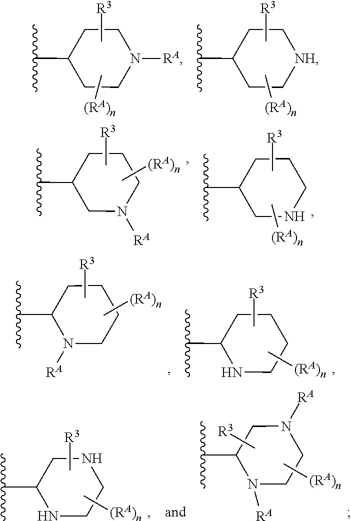
and each n is independently 0, 1, 2, 3, or 4.

10. The compound of claim 1, or a pharmaceutically acceptable salt, or solvate thereof, wherein L¹ is —CH₂—, C(═O), O, S, S(═O), S(═O)₂, or NR⁴; or L² is —CH₂—, C(═O), O, S, S(═O), S(═O)₂, or NR₄.

11. (canceled)

12. The compound of claim 1, or a pharmaceutically acceptable salt, or solvate thereof, wherein L³ is absent, —CH₂—, —CH₂—CH₂—, or —CH₂—CH₂—CH₂—.

13. The compound of claim 1, or a pharmaceutically acceptable salt, or solvate thereof, wherein X is —CH₂— or C(═O); or Y is —CH₂— or C(═O).

14.-15. (canceled)

16. The compound of claim 1, or a pharmaceutically acceptable salt, or solvate thereof, wherein R¹¹ is hydrogen.

17. The compound of claim 1, or a pharmaceutically acceptable salt, or solvate thereof, wherein R⁹ and R¹⁰ are each H; or R⁹ is H and R¹⁰ is —C₁-C₄alkylene-(optionally substituted phenyl) or —C₁-C₄alkylene-(optionally substituted heteroaryl).

18. (canceled)

19. The compound of claim 1, or a pharmaceutically acceptable salt, or solvate thereof, wherein R¹ is an unsubstituted phenyl or substituted phenyl; or R² is an unsubstituted phenyl or substituted phenyl.

20.-83. (canceled)

84. A compound of Formula (Va), or a pharmaceutically acceptable salt, or solvate thereof: 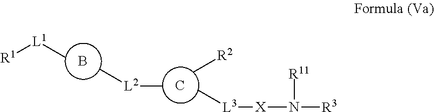
wherein, 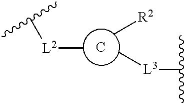
is a bicyclic heteroaryl that is selected from the following structures: 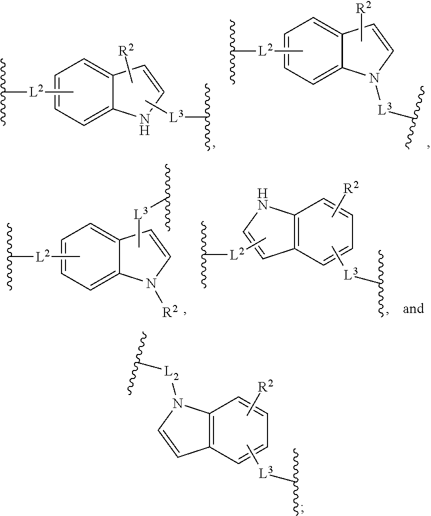
L¹ and L² are each independently absent, an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
Ring B is an optionally substituted monocyclic or bicyclic heterocycloalkyl ring containing at least one N with the proviso that Ring B is not: 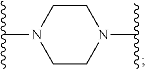 [SMILES: *N1CCN(*)CC1]
wherein if Ring B is substituted, then Ring B is substituted with at least one R^{B};
each R^{B} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
L³ is absent, an optionally substituted C₁-C₆heteroalkylene, an optionally substituted C₁-C₆alkylene, an optionally substituted phenylene, an optionally substituted C₃-C₆cycloalkylene, an optionally substituted —C₃-C₆cycloalkylene-(optionally substituted C₁-C₄alkylene), or an optionally substituted —C₁-C₄alkylene-(optionally substituted C₃-C₆cycloalkylene);
wherein if L³ is substituted then L³ is substituted with at least one R^{D};
each R^{D} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
R³ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, —C₁-C₄alkylene-(optionally substituted heteroaryl), —CH₂C(═O)R¹⁵, —C(═O)R¹⁵, or —CO₂R¹⁶;
R⁴ is hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹⁵ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; and
each R¹⁶ is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl.

85. The compound of claim 84, or a pharmaceutically acceptable salt, or solvate thereof, wherein:

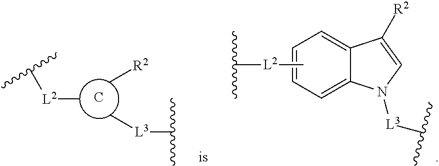

86.-87. (canceled)

88. The compound of claim 84, or a pharmaceutically acceptable salt, or solvate thereof, wherein:

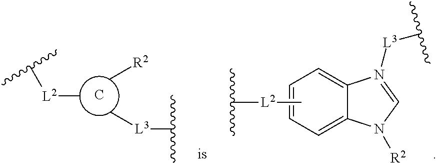

89.-90. (canceled)

91. The compound of claim 84, or a pharmaceutically acceptable salt, or solvate thereof, wherein 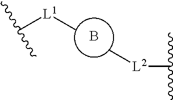
is selected from the following: 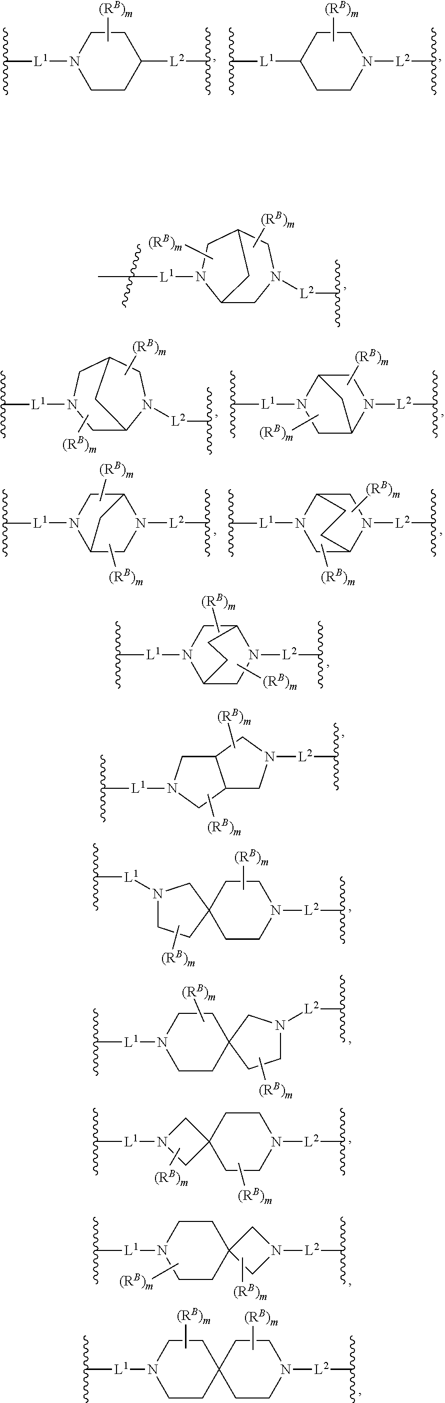 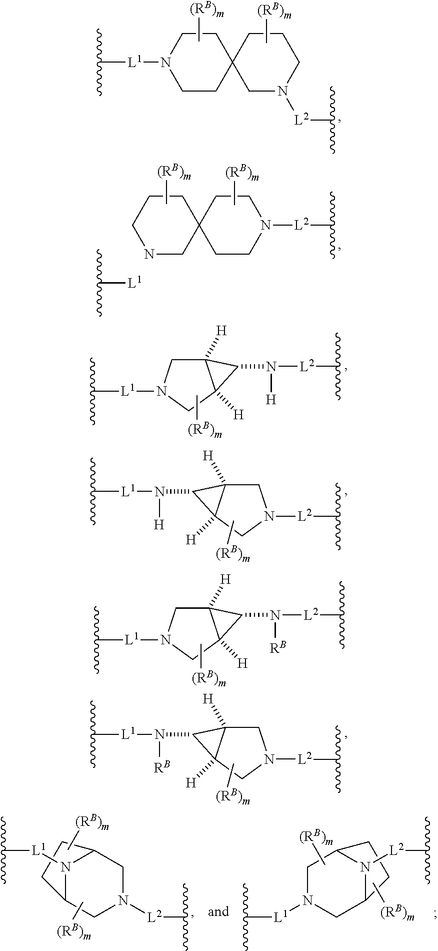
and each m is independently 0, 1, 2, 3, or 4.

92. The compound of claim 84, or a pharmaceutically acceptable salt, or solvate thereof, wherein L¹ is —CH₂—, C(═O), O, S, S(═O), S(═O)₂, or NR⁴; or L² is —CH₂—, C(═O), O, S, S(═O), S(═O)₂, or NR⁴.

93. (canceled)

94. The compound of claim 84, or a pharmaceutically acceptable salt, or solvate thereof, wherein L³ is absent, —CH₂—, —CH₂—CH₂—, or —CH₂—CH₂—CH₂—; or X is —CH₂— or C(═O).

95.-96. (canceled)

97. The compound of claim 84, or a pharmaceutically acceptable salt, or solvate thereof, wherein R¹¹ and R³ are each independently hydrogen, optionally substituted C₁-C₆alkyl, optionally substituted C₁-C₆heteroalkyl, —CH₂C(═O)R¹⁵, —C(═O)R¹⁵, or —CO₂R¹⁶.

98. The compound of claim 84, or a pharmaceutically acceptable salt, or solvate thereof, wherein R¹ is an unsubstituted phenyl or a substituted phenyl; or R² is an unsubstituted phenyl or substituted phenyl.

99.-101. (canceled)

102. The compound of claim 84 having the following structure of Formula (Vf), or a pharmaceutically acceptable salt, or solvate thereof:

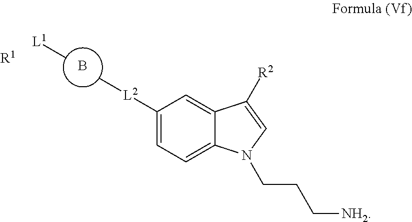

103. The compound of claim 84 having the following structure of Formula (Vg), or a pharmaceutically acceptable salt, or solvate thereof: 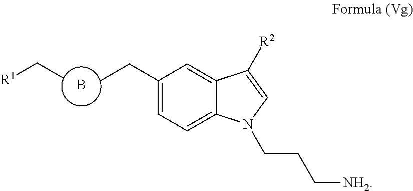
wherein R¹ and R² are each independently optionally substituted aryl; and 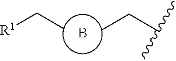
is selected from

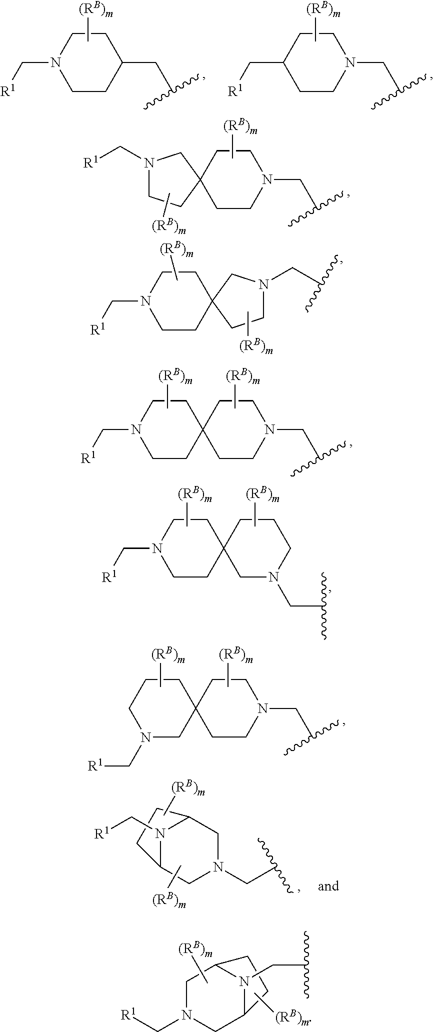

104. The compound of claim 84, or a pharmaceutically acceptable salt, or solvate thereof, wherein the compound of Formula (Va) is selected from:

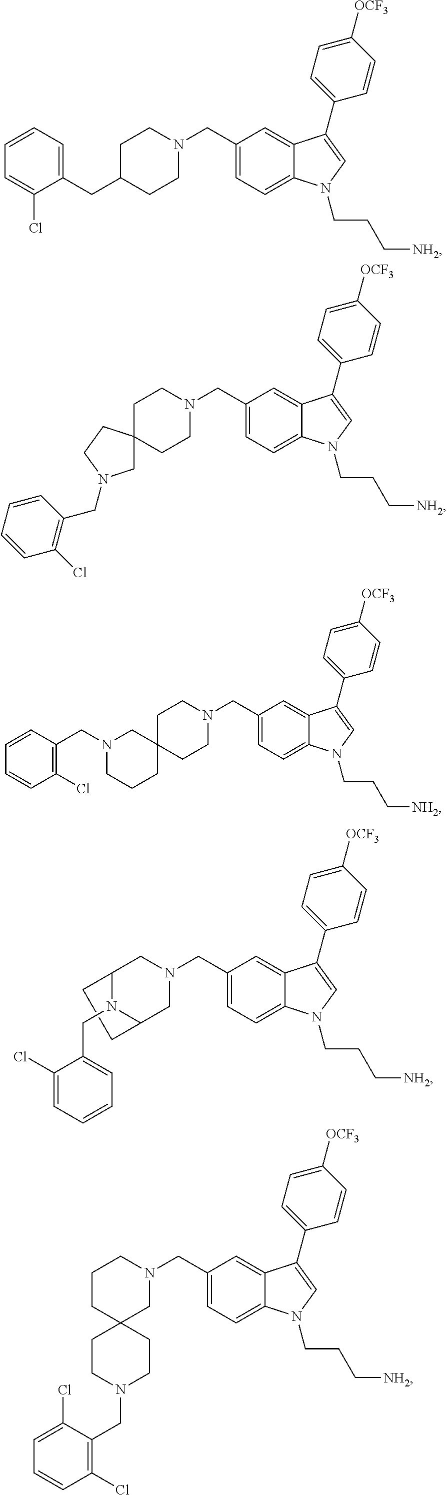

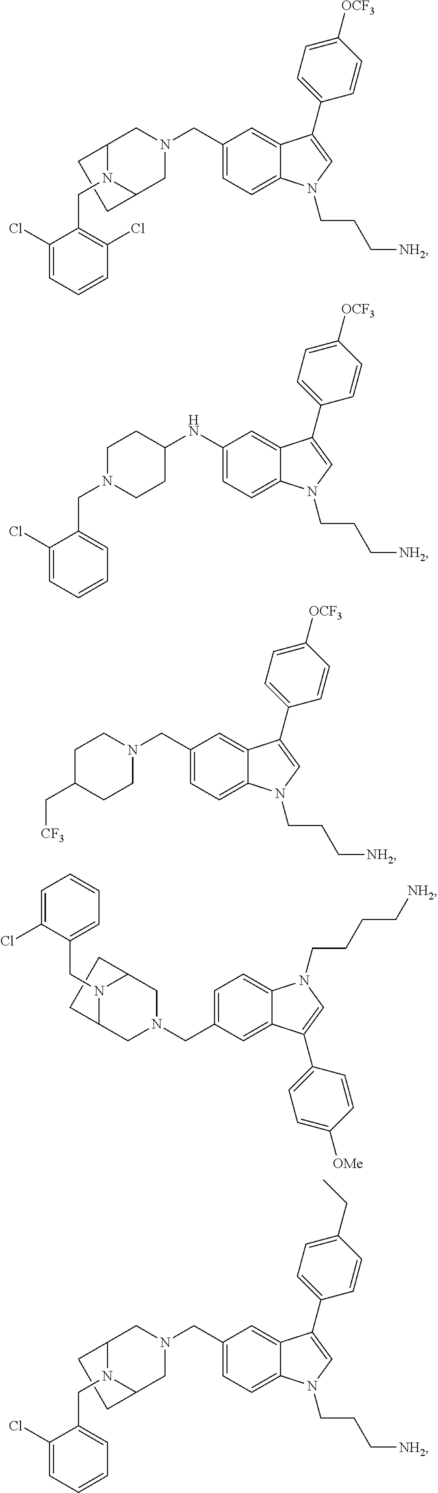

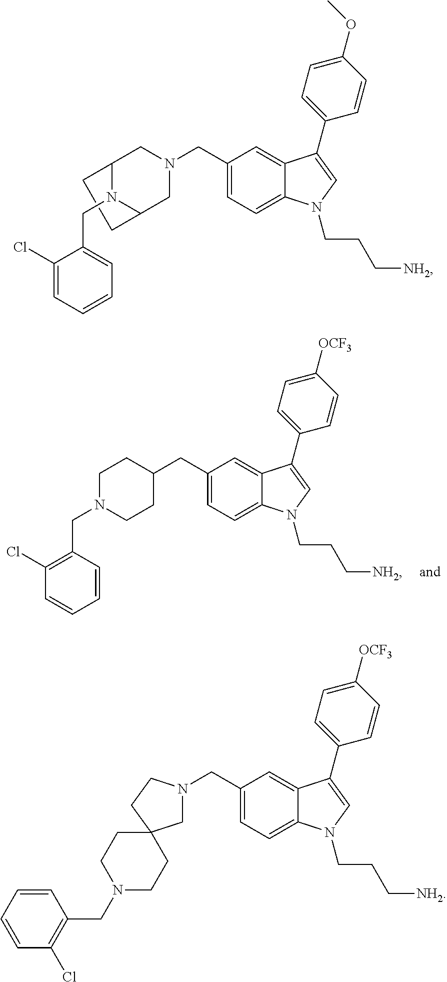 [SMILES: COc1ccc(-c2cn(CCCN)c3ccc(CN4CC5CCC(C4)N5Cc4ccccc4Cl)cc23)cc1 ; NCCCn1cc(-c2ccc(OC(F)(F)F)cc2)c2cc(CC3CCN(Cc4ccccc4Cl)CC3)ccc21 ; NCCCn1cc(-c2ccc(OC(F)(F)F)cc2)c2cc(CN3CCC4(CCN(Cc5ccccc5Cl)CC4)C3)ccc21]

105. The compound of claim 84, or a pharmaceutically acceptable salt, or solvate thereof, wherein the compound of Formula (Va) is selected from:

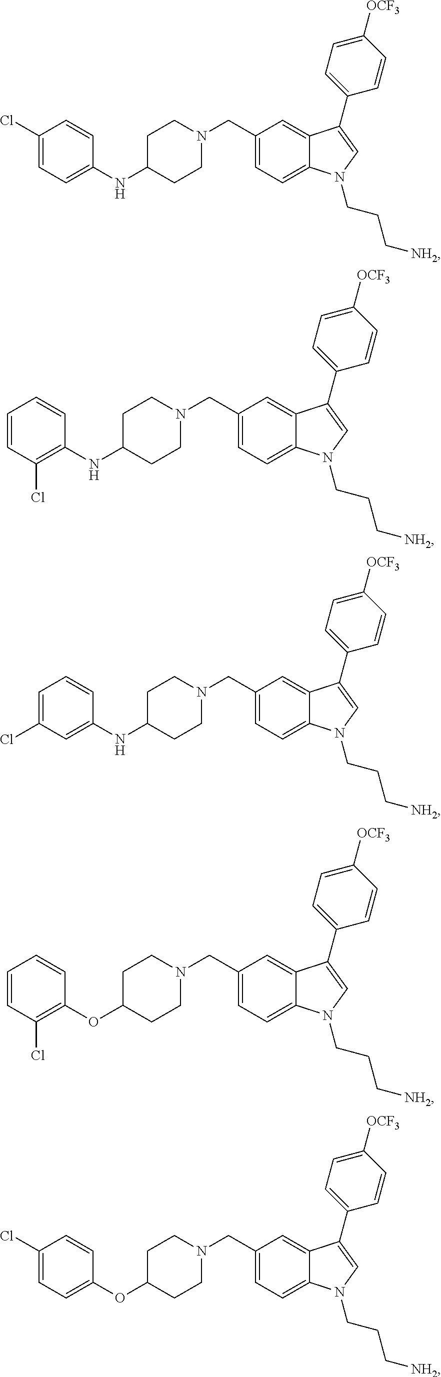

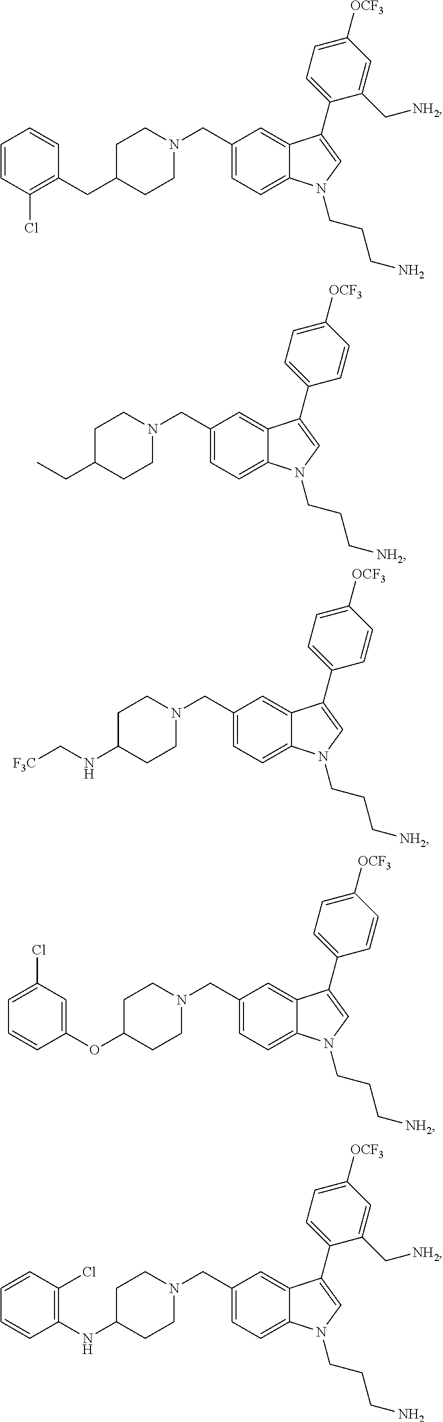

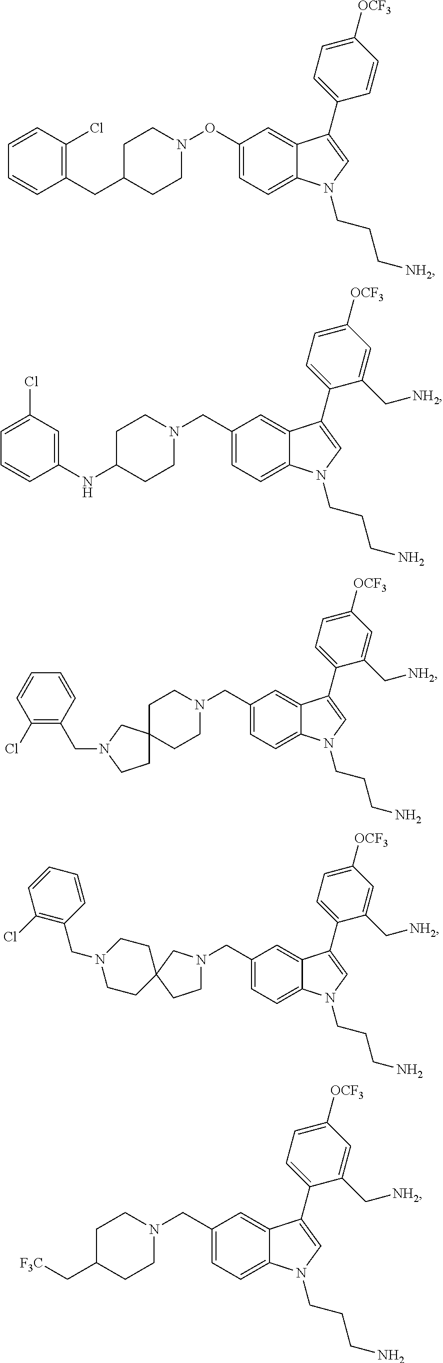

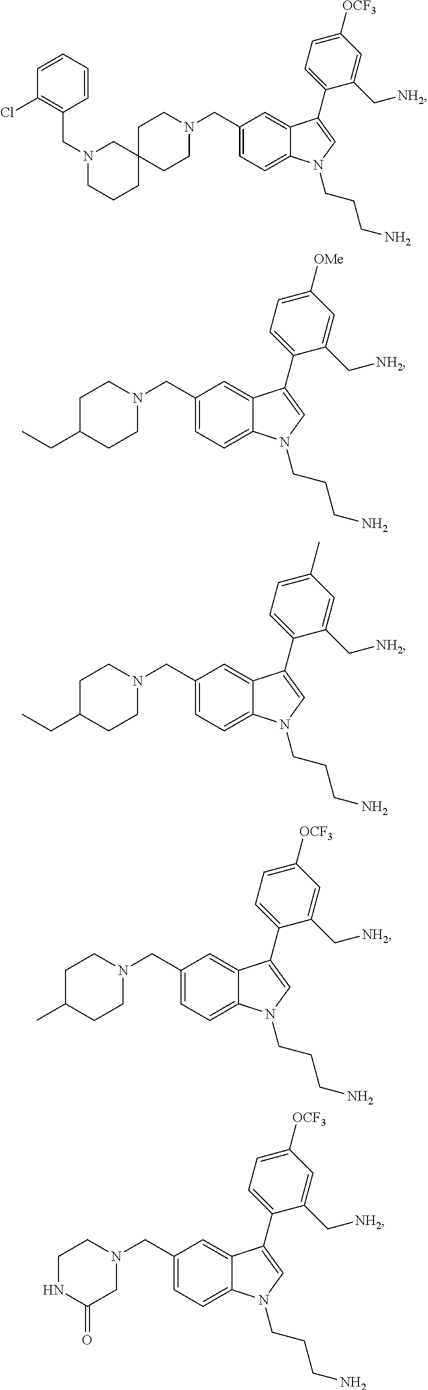

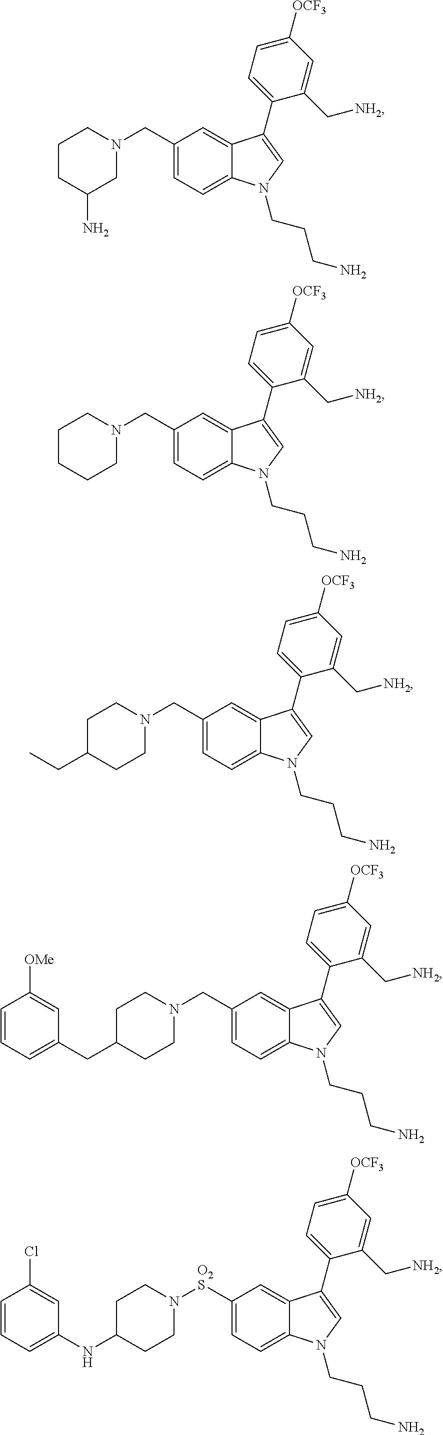

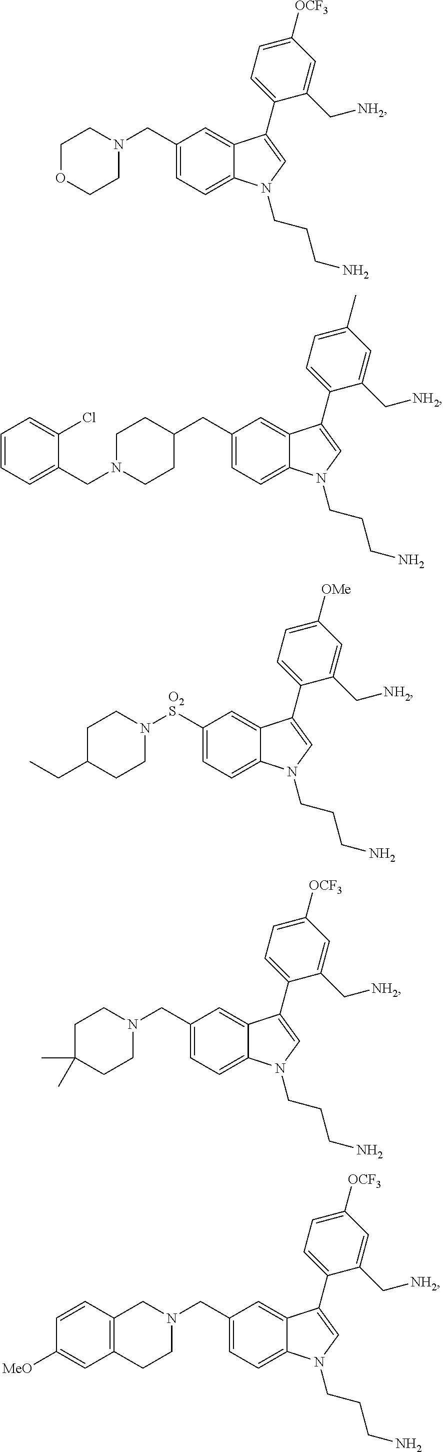

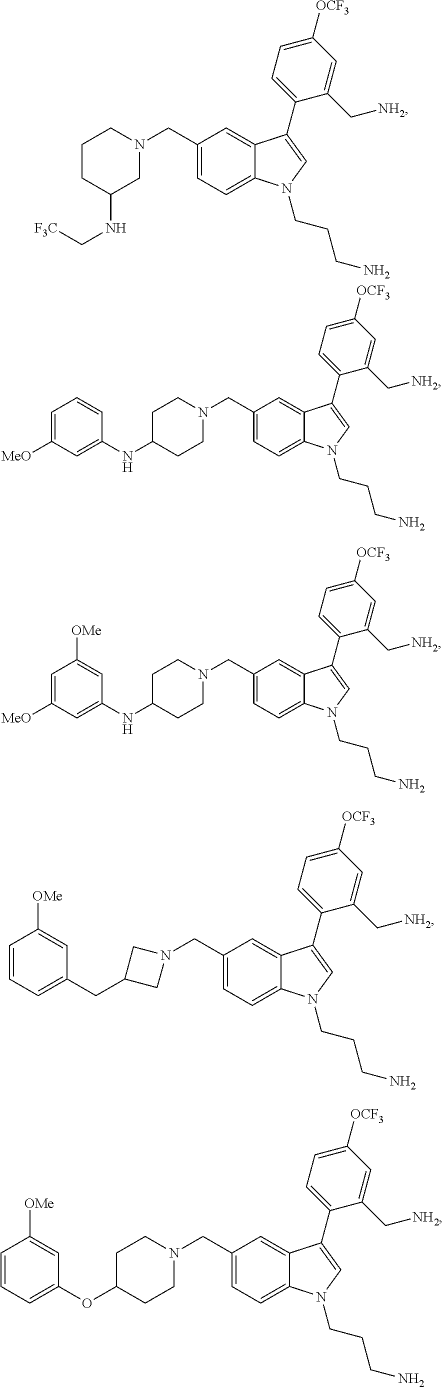

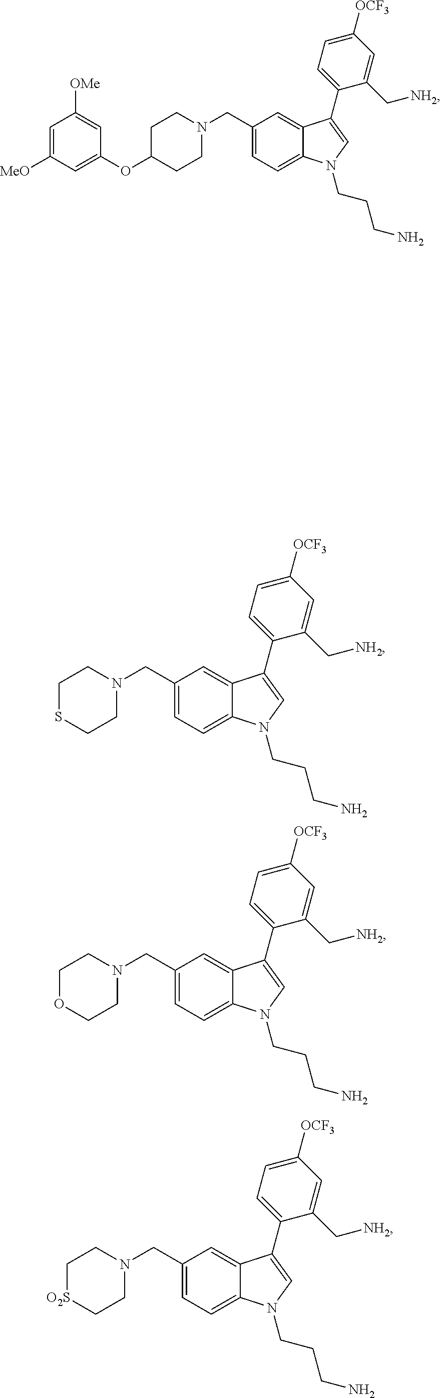

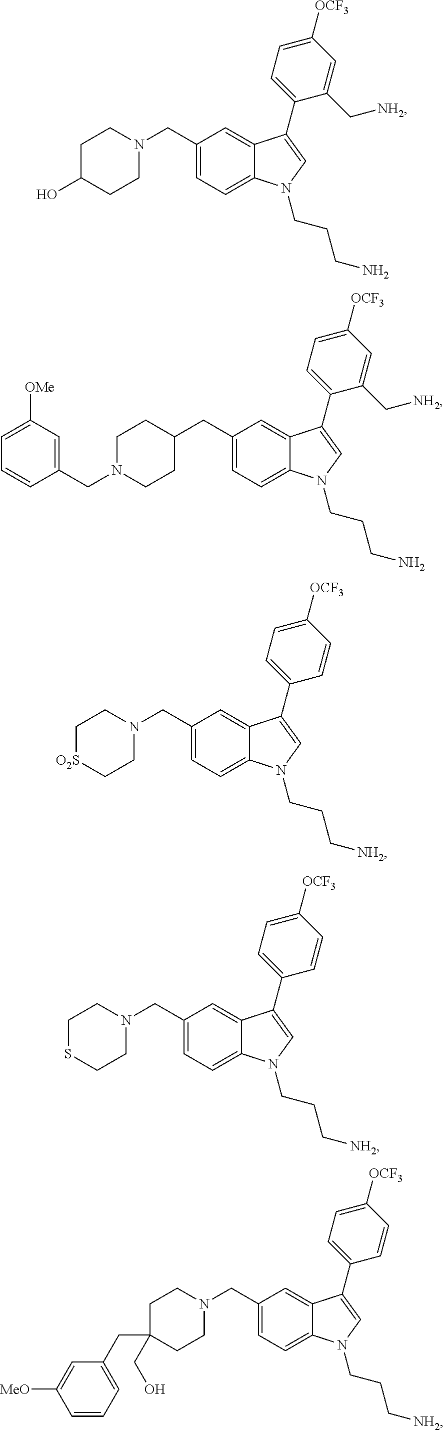

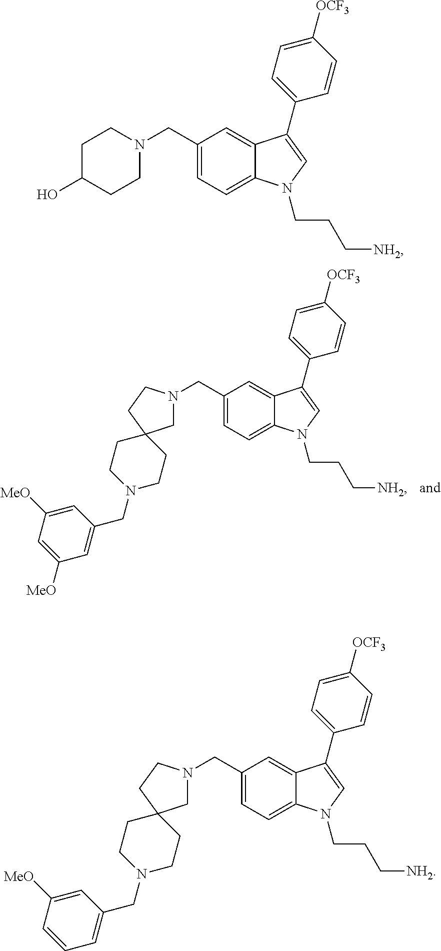

106.-123. (canceled)

124. A compound of Formula (VIIa), or a pharmaceutically acceptable salt, or solvate thereof: 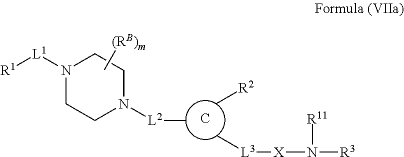
wherein, 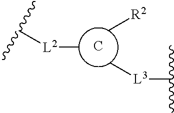
is a bicyclic heteroaryl that is selected from the following structures: 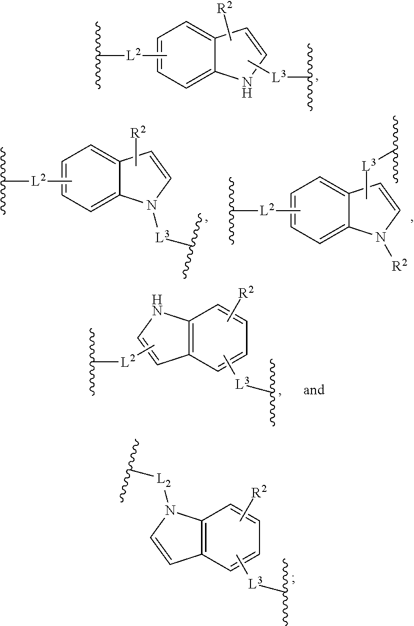
L¹ and L² are each independently absent, an optionally substituted C₁-C₆alkylene, an optionally substituted C₁-C₆heteroalkylene, an optionally substituted C₃-C₆cycloalkylene, C(═O), O, S, S(═O), S(═O)₂, or NR⁴;
R¹ is hydrogen, an optionally substituted C₁-C₆alkyl, an optionally substituted C₁-C₆heteroalkyl, an optionally substituted C₃-C₆cycloalkyl, an optionally substituted C₂-C₁₀heterocycloalkyl, an optionally substituted aryl, optionally substituted heterocycloalkyl, or optionally substituted heteroaryl;
R² is an optionally substituted aryl, optionally substituted heterocycloalkyl, or optionally substituted heteroaryl;
each R^{B} is independently optionally substituted C₁-C₆alkyl, optionally substituted C₃-C₆cycloalkyl, optionally substituted aryl, —C₁-C₄alkylene-(optionally substituted aryl), optionally substituted heteroaryl, or —C₁-C₄alkylene-(optionally substituted heteroaryl);
L³ is an unsubstituted C₁-C₆alkylene;
X is an optionally substituted C₃-C₆cycloalkylene, —C(R⁵)(R⁶)—, or C(═O);
wherein if X is substituted then X is substituted with at least one R^{E};
R⁵ and R⁶ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl); or
R⁵ and R⁶ are taken together with carbon atom to which they are attached to form an optionally substituted carbocycloalkyl; wherein if the carbocycloalkyl is substituted then the carbocycloalkyl is substituted with at least one R^{E};
each R^{E} is independently halogen, —CN, —OR¹², —SR¹², —S(═O)R¹³, —S(═O)₂R¹³, —S(═O)₂N(R¹²)₂, —NR¹⁴S(═O)₂R¹³, —C(═O)R¹³, —OC(═O)R¹³, —CO₂R¹², —OCO₂R¹³, —N(R¹²)₂, —OC(═O)N(R¹²)₂, —NR¹⁴C(═O)R¹³, —NR¹⁴C(═O)OR¹³, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
R³ and R¹¹ are each independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, —C₁-C₄alkylene-(optionally substituted heteroaryl), —CH₂C(═O)R¹⁵, —C(═O)R¹⁵, or —CO₂R¹⁶;
R⁴ is hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹² is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl; or
two R¹² are taken together with the N atom to which they are attached to form an optionally substituted heterocycloalkyl ring;
each R¹³ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁴ is independently hydrogen, optionally substituted C₁-C₆alkyl, optionally substituted C₁-C₆heteroalkyl, optionally substituted C₃-C₆cycloalkyl, optionally substituted C₂-C₁₀heterocycloalkyl, optionally substituted aryl, —C₁-C₄alkylene-(optionally substituted aryl), optionally substituted heteroaryl, or —C₁-C₄alkylene-(optionally substituted heteroaryl);
each R¹⁵ is independently optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each R¹⁶ is independently hydrogen, optionally substituted C₁-C₆alkyl, optionally substituted C₁-C₆heteroalkyl, optionally substituted C₃-C₁₀cycloalkyl, optionally substituted C₂-C₁₀heterocycloalkyl, optionally substituted aryl, or optionally substituted heteroaryl;
each m is independently 0, 1, 2, 3, or 4; and
with the provision that the compound is not

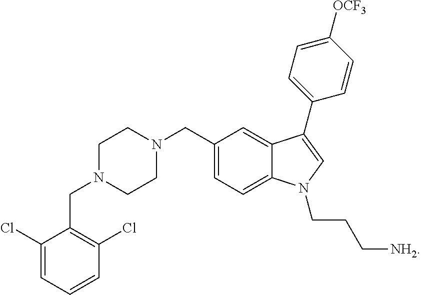 [SMILES: NCCCn1cc(-c2ccc(OC(F)(F)F)cc2)c2cc(CN3CCN(Cc4c(Cl)cccc4Cl)CC3)ccc21]

125. The compound of claim 124, or a pharmaceutically acceptable salt, or solvate thereof, wherein:

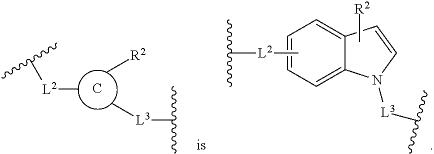

126.-127. (canceled)

128. The compound of claim 124, or a pharmaceutically acceptable salt, or solvate thereof, wherein:

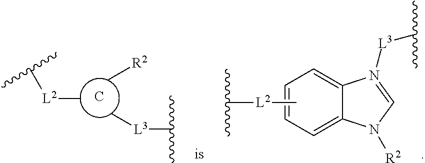

129.-130. (canceled)

131. The compound of claim 124, or a pharmaceutically acceptable salt, or solvate thereof, wherein L¹ is —CH₂—, C(═O), O, S, S(═O), S(═O)₂, or NR⁴ or L² is —CH₂—, C(═O), O, S, S(═O), S(═O)₂, or NR⁴.

132. (canceled)

133. The compound of claim 124, or a pharmaceutically acceptable salt, or solvate thereof, wherein L³ is —CH₂—, —CH₂CH₂—, or —CH₂—CH₂—CH₂— or X is —CH— or C(═O).

134.-135. (canceled)

136. The compound of claim 124, or a pharmaceutically acceptable salt, or solvate thereof, wherein R¹¹ and R³ are each independently hydrogen, optionally substituted C₁-C₆alkyl, optionally substituted C₁-C₆heteroalkyl, —CH₂C(═O)R¹⁵, —C(═O)R¹⁵, or —CO₂R¹⁶.

137. The compound of claim 124, or a pharmaceutically acceptable salt, or solvate thereof, wherein R¹ is an unsubstituted phenyl or a substituted phenyl; or R² is an unsubstituted phenyl or substituted phenyl.

138.-140. (canceled)

141. The compound of claim 124 having the following structure of Formula (VIIf), or a pharmaceutically acceptable salt, or solvate thereof: 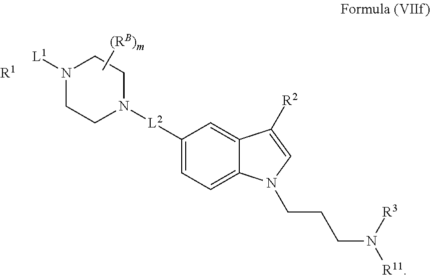
wherein R¹ and R² are each independently optionally substituted aryl.

142. The compound of claim 124 having the following structure of Formula (VIIg), or a pharmaceutically acceptable salt, or solvate thereof: 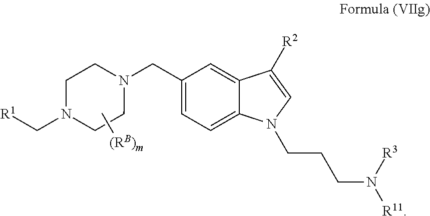
wherein R¹ and R² are each independently optionally substituted aryl.

143. The compound of claim 124, or a pharmaceutically acceptable salt, or solvate thereof, wherein the compound of Formula (VIIa) is selected from:

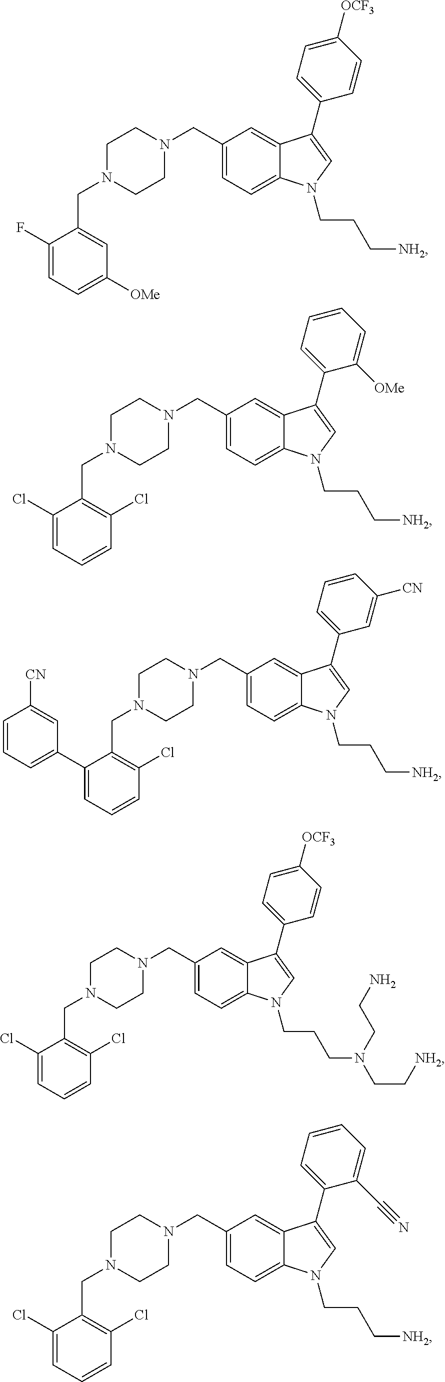 [SMILES: COc1ccc(F)c(CN2CCN(Cc3ccc4c(c3)c(-c3ccc(OC(F)(F)F)cc3)cn4CCCN)CC2)c1 ; COc1ccccc1-c1cn(CCCN)c2ccc(CN3CCN(Cc4c(Cl)cccc4Cl)CC3)cc12 ; N#Cc1cccc(-c2cccc(Cl)c2CN2CCN(Cc3ccc4c(c3)c(-c3cccc(C#N)c3)cn4CCCN)CC2)c1 ; NCCN(CCN)CCCn1cc(-c2ccc(OC(F)(F)F)cc2)c2cc(CN3CCN(Cc4c(Cl)cccc4Cl)CC3)ccc21 ; N#Cc1ccccc1-c1cn(CCCN)c2ccc(CN3CCN(Cc4c(Cl)cccc4Cl)CC3)cc12]

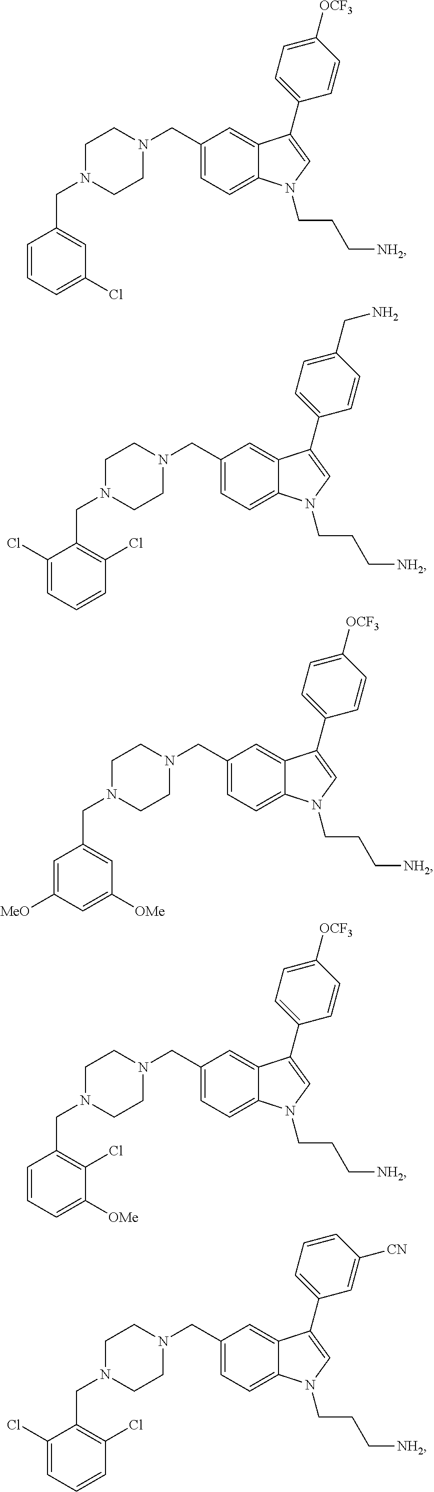 [SMILES: NCCCn1cc(-c2ccc(OC(F)(F)F)cc2)c2cc(CN3CCN(Cc4cccc(Cl)c4)CC3)ccc21 ; NCCCn1cc(-c2ccc(CN)cc2)c2cc(CN3CCN(Cc4c(Cl)cccc4Cl)CC3)ccc21 ; COc1cc(CN2CCN(Cc3ccc4c(c3)c(-c3ccc(OC(F)(F)F)cc3)cn4CCCN)CC2)cc(OC)c1 ; COc1cccc(CN2CCN(Cc3ccc4c(c3)c(-c3ccc(OC(F)(F)F)cc3)cn4CCCN)CC2)c1Cl ; N#Cc1cccc(-c2cn(CCCN)c3ccc(CN4CCN(Cc5c(Cl)cccc5Cl)CC4)cc23)c1]

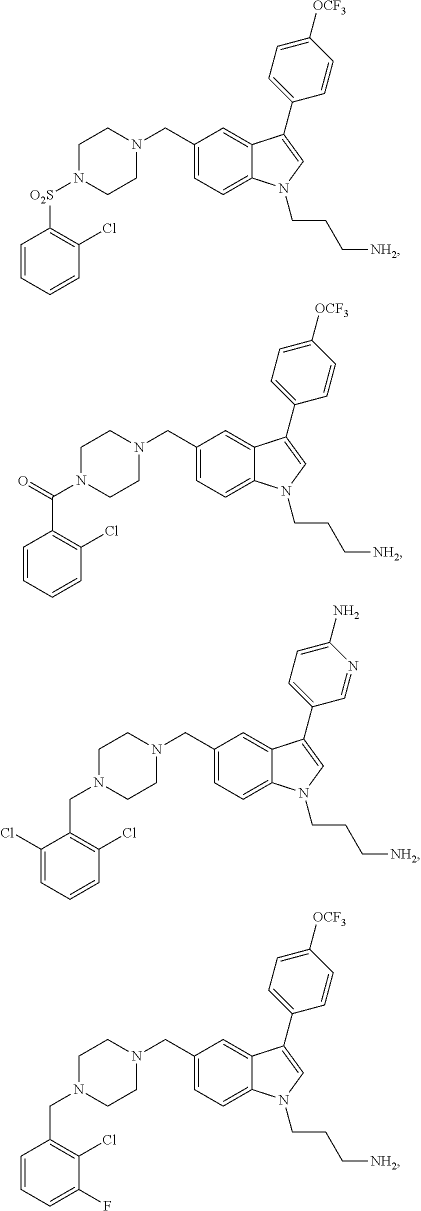 [SMILES: NCCCn1cc(-c2ccc(OC(F)(F)F)cc2)c2cc(CN3CCN(S(=O)(=O)c4ccccc4Cl)CC3)ccc21 ; NCCCn1cc(-c2ccc(OC(F)(F)F)cc2)c2cc(CN3CCN(C(=O)c4ccccc4Cl)CC3)ccc21 ; NCCCn1cc(-c2ccc(N)nc2)c2cc(CN3CCN(Cc4c(Cl)cccc4Cl)CC3)ccc21 ; NCCCn1cc(-c2ccc(OC(F)(F)F)cc2)c2cc(CN3CCN(Cc4cccc(F)c4Cl)CC3)ccc21]

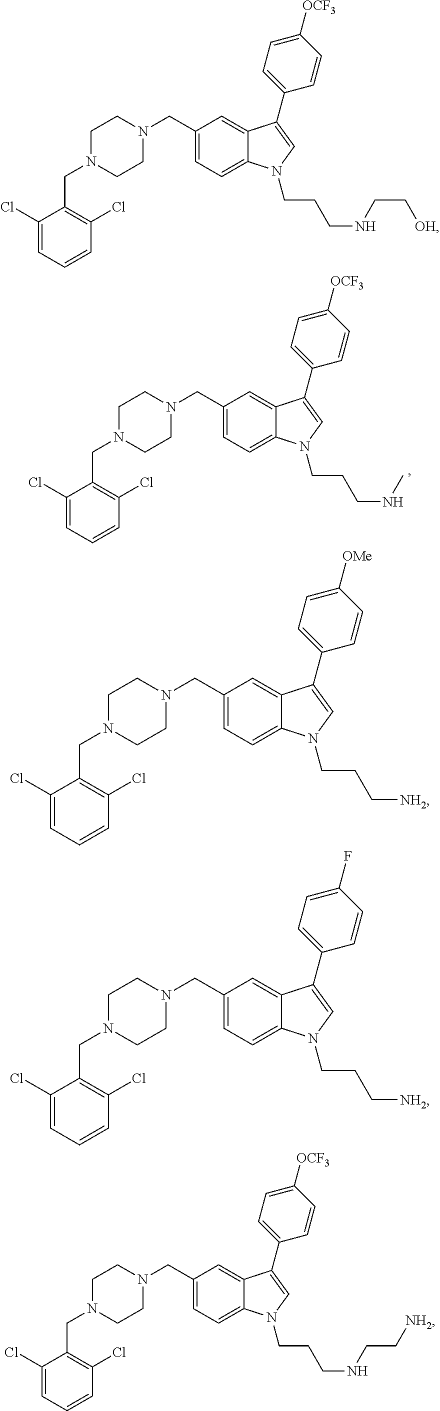 [SMILES: OCCNCCCn1cc(-c2ccc(OC(F)(F)F)cc2)c2cc(CN3CCN(Cc4c(Cl)cccc4Cl)CC3)ccc21 ; CNCCCn1cc(-c2ccc(OC(F)(F)F)cc2)c2cc(CN3CCN(Cc4c(Cl)cccc4Cl)CC3)ccc21 ; COc1ccc(-c2cn(CCCN)c3ccc(CN4CCN(Cc5c(Cl)cccc5Cl)CC4)cc23)cc1 ; NCCCn1cc(-c2ccc(F)cc2)c2cc(CN3CCN(Cc4c(Cl)cccc4Cl)CC3)ccc21 ; NCCNCCCn1cc(-c2ccc(OC(F)(F)F)cc2)c2cc(CN3CCN(Cc4c(Cl)cccc4Cl)CC3)ccc21]

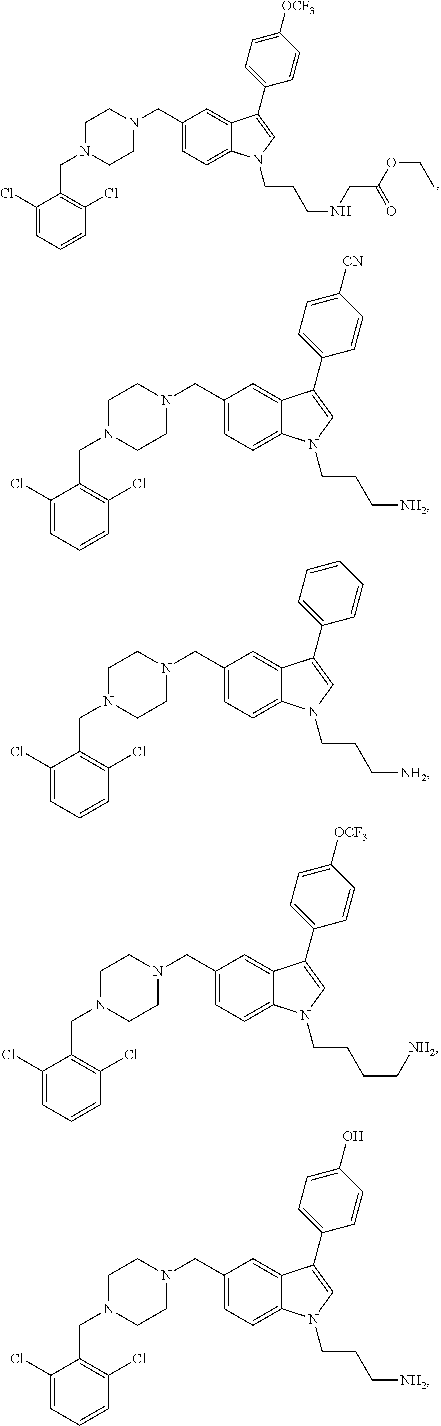 [SMILES: CCOC(=O)CNCCCn1cc(-c2ccc(OC(F)(F)F)cc2)c2cc(CN3CCN(Cc4c(Cl)cccc4Cl)CC3)ccc21 ; N#Cc1ccc(-c2cn(CCCN)c3ccc(CN4CCN(Cc5c(Cl)cccc5Cl)CC4)cc23)cc1 ; NCCCn1cc(-c2ccccc2)c2cc(CN3CCN(Cc4c(Cl)cccc4Cl)CC3)ccc21 ; NCCCCn1cc(-c2ccc(OC(F)(F)F)cc2)c2cc(CN3CCN(Cc4c(Cl)cccc4Cl)CC3)ccc21 ; NCCCn1cc(-c2ccc(O)cc2)c2cc(CN3CCN(Cc4c(Cl)cccc4Cl)CC3)ccc21]

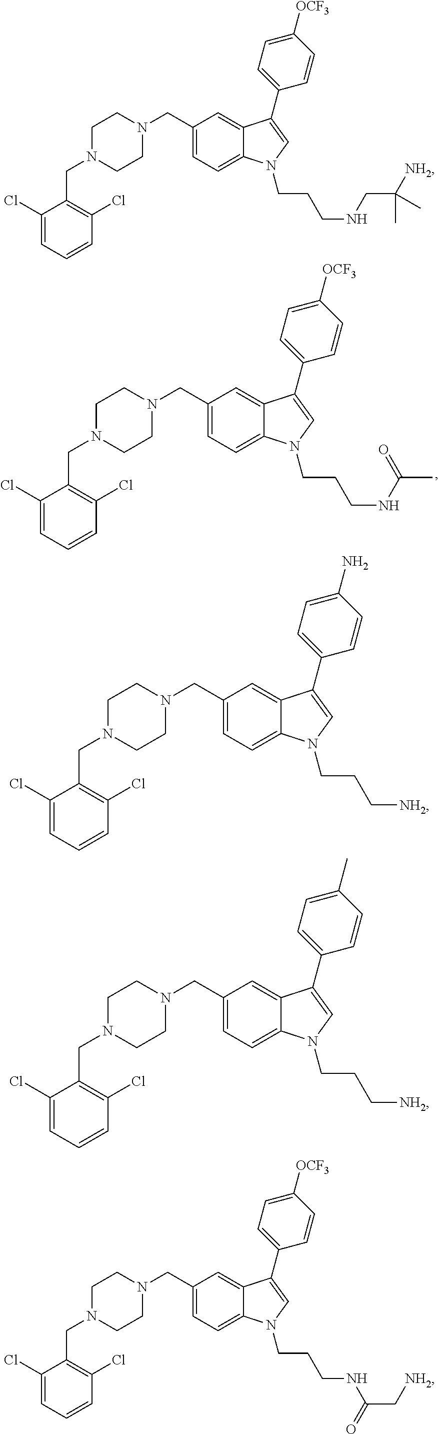 [SMILES: CC(C)(N)CNCCCn1cc(-c2ccc(OC(F)(F)F)cc2)c2cc(CN3CCN(Cc4c(Cl)cccc4Cl)CC3)ccc21 ; CC(=O)NCCCn1cc(-c2ccc(OC(F)(F)F)cc2)c2cc(CN3CCN(Cc4c(Cl)cccc4Cl)CC3)ccc21 ; NCCCn1cc(-c2ccc(N)cc2)c2cc(CN3CCN(Cc4c(Cl)cccc4Cl)CC3)ccc21 ; Cc1ccc(-c2cn(CCCN)c3ccc(CN4CCN(Cc5c(Cl)cccc5Cl)CC4)cc23)cc1 ; NCC(=O)NCCCn1cc(-c2ccc(OC(F)(F)F)cc2)c2cc(CN3CCN(Cc4c(Cl)cccc4Cl)CC3)ccc21]

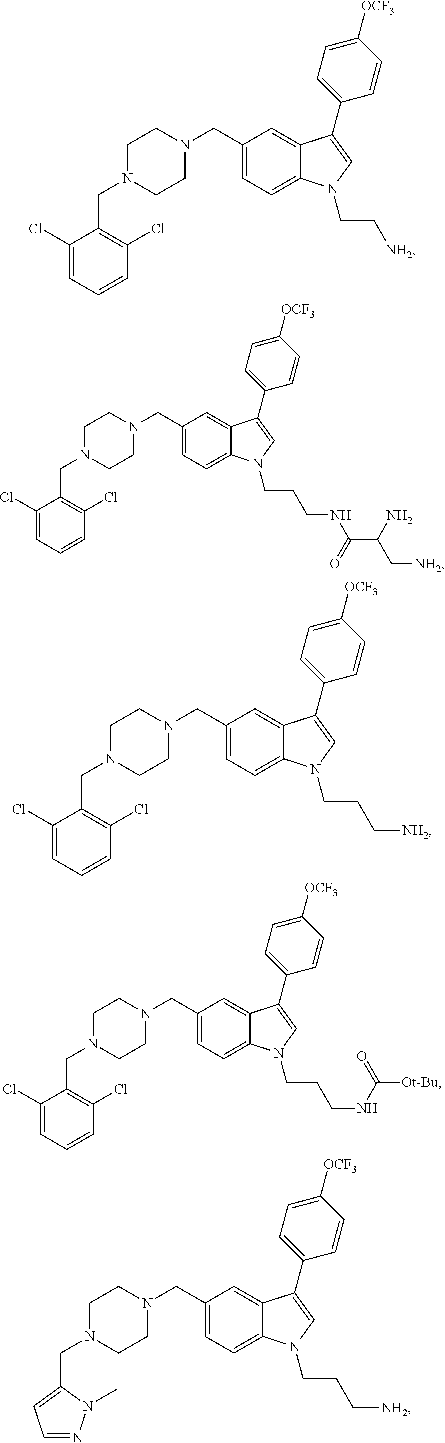

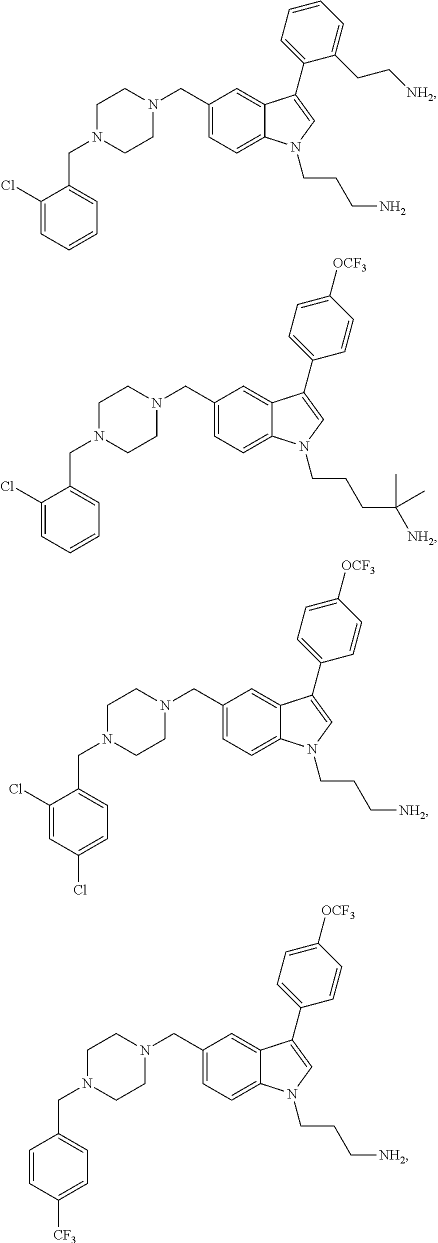 [SMILES: NCCCn1cc(-c2ccccc2CCN)c2cc(CN3CCN(Cc4ccccc4Cl)CC3)ccc21 ; CC(C)(N)CCCn1cc(-c2ccc(OC(F)(F)F)cc2)c2cc(CN3CCN(Cc4ccccc4Cl)CC3)ccc21 ; NCCCn1cc(-c2ccc(OC(F)(F)F)cc2)c2cc(CN3CCN(Cc4ccc(Cl)cc4Cl)CC3)ccc21 ; NCCCn1cc(-c2ccc(OC(F)(F)F)cc2)c2cc(CN3CCN(Cc4ccc(C(F)(F)F)cc4)CC3)ccc21]

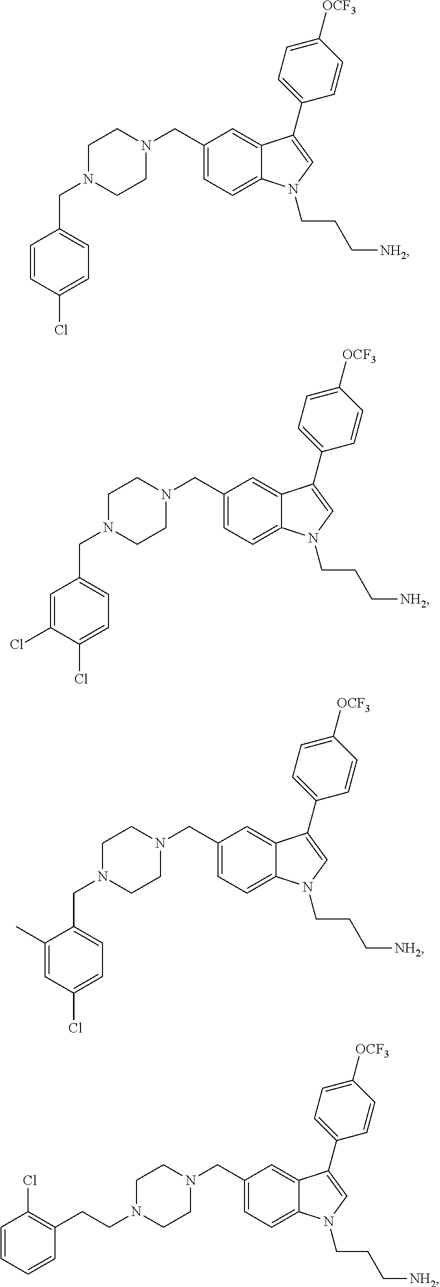 [SMILES: NCCCn1cc(-c2ccc(OC(F)(F)F)cc2)c2cc(CN3CCN(Cc4ccc(Cl)cc4)CC3)ccc21 ; NCCCn1cc(-c2ccc(OC(F)(F)F)cc2)c2cc(CN3CCN(Cc4ccc(Cl)c(Cl)c4)CC3)ccc21 ; Cc1cc(Cl)ccc1CN1CCN(Cc2ccc3c(c2)c(-c2ccc(OC(F)(F)F)cc2)cn3CCCN)CC1 ; NCCCn1cc(-c2ccc(OC(F)(F)F)cc2)c2cc(CN3CCN(CCc4ccccc4Cl)CC3)ccc21]

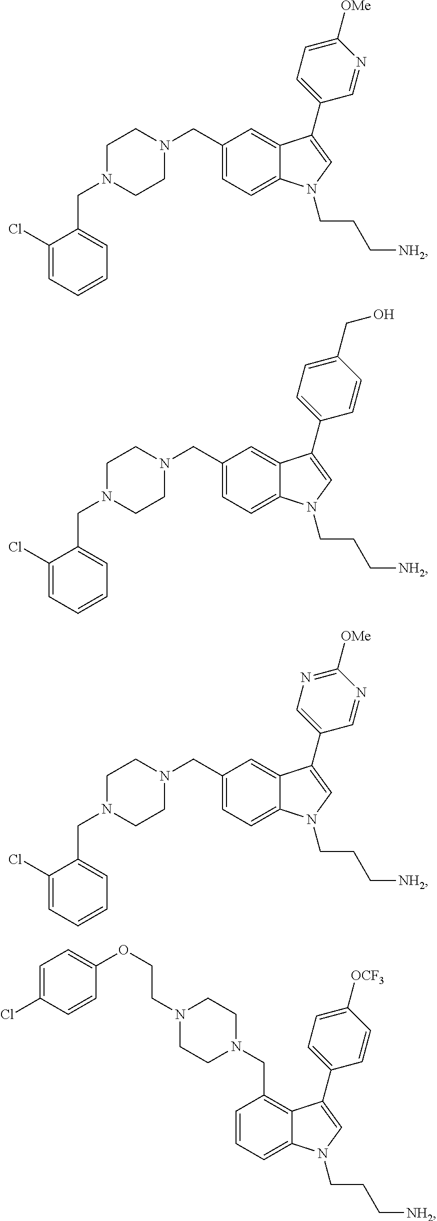

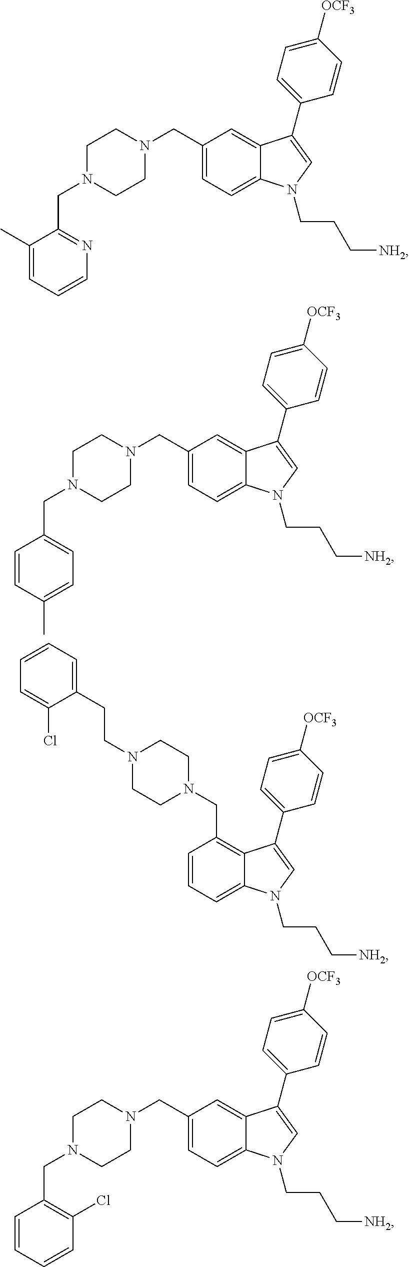

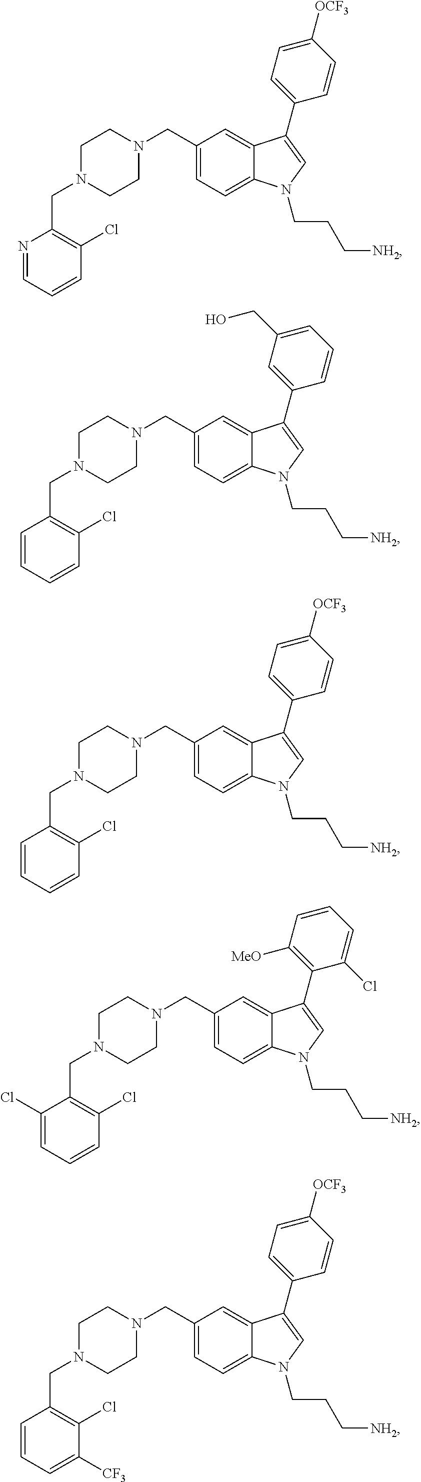 [SMILES: NCCCn1cc(-c2ccc(OC(F)(F)F)cc2)c2cc(CN3CCN(Cc4ncccc4Cl)CC3)ccc21 ; NCCCn1cc(-c2cccc(CO)c2)c2cc(CN3CCN(Cc4ccccc4Cl)CC3)ccc21 ; NCCCn1cc(-c2ccc(OC(F)(F)F)cc2)c2cc(CN3CCN(Cc4ccccc4Cl)CC3)ccc21 ; COc1cccc(Cl)c1-c1cn(CCCN)c2ccc(CN3CCN(Cc4c(Cl)cccc4Cl)CC3)cc12 ; NCCCn1cc(-c2ccc(OC(F)(F)F)cc2)c2cc(CN3CCN(Cc4cccc(C(F)(F)F)c4Cl)CC3)ccc21]

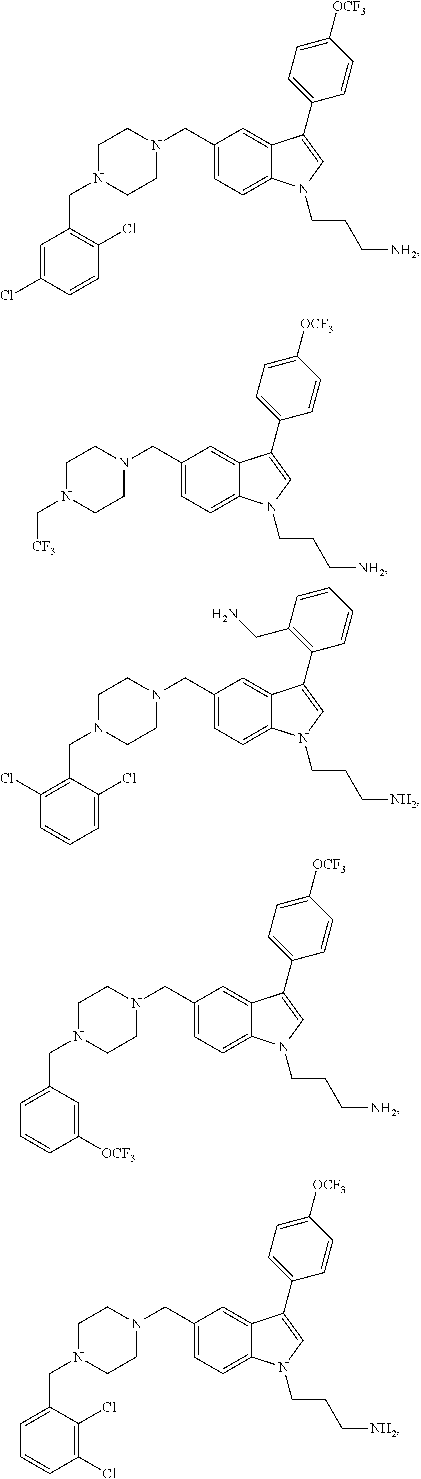

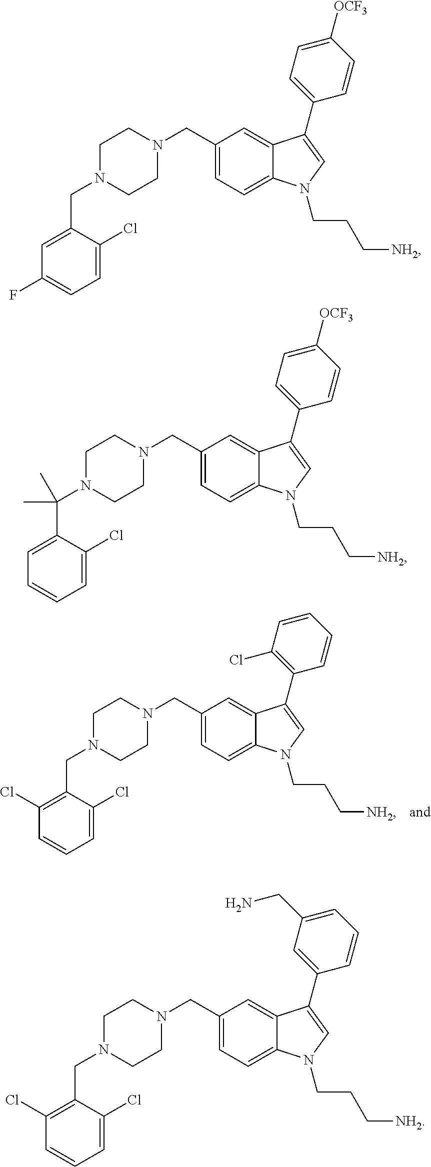 [SMILES: NCCCn1cc(-c2ccc(OC(F)(F)F)cc2)c2cc(CN3CCN(Cc4cc(F)ccc4Cl)CC3)ccc21 ; CC(C)(c1ccccc1Cl)N1CCN(Cc2ccc3c(c2)c(-c2ccc(OC(F)(F)F)cc2)cn3CCCN)CC1 ; NCCCn1cc(-c2ccccc2Cl)c2cc(CN3CCN(Cc4c(Cl)cccc4Cl)CC3)ccc21 ; NCCCn1cc(-c2cccc(CN)c2)c2cc(CN3CCN(Cc4c(Cl)cccc4Cl)CC3)ccc21]

144. The compound of claim 124, or a pharmaceutically acceptable salt, or solvate thereof, wherein the compound of Formula (VIIa) is selected from:

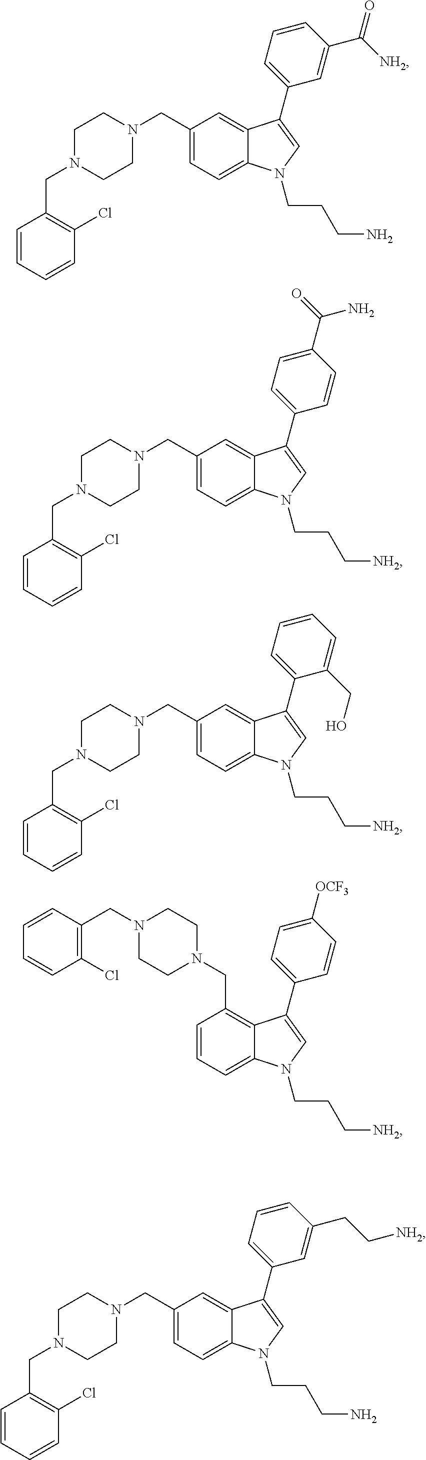

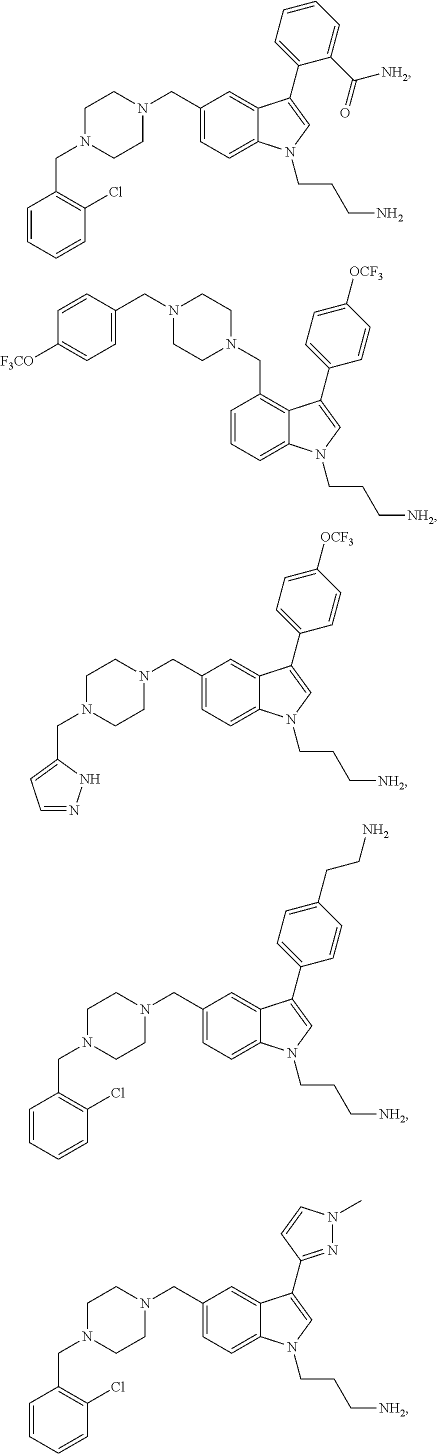 [SMILES: NCCCn1cc(-c2ccccc2C(N)=O)c2cc(CN3CCN(Cc4ccccc4Cl)CC3)ccc21 ; NCCCn1cc(-c2ccc(OC(F)(F)F)cc2)c2c(CN3CCN(Cc4ccc(OC(F)(F)F)cc4)CC3)cccc21 ; NCCCn1cc(-c2ccc(OC(F)(F)F)cc2)c2cc(CN3CCN(Cc4ccn[nH]4)CC3)ccc21 ; NCCCn1cc(-c2ccc(CCN)cc2)c2cc(CN3CCN(Cc4ccccc4Cl)CC3)ccc21 ; Cn1ccc(-c2cn(CCCN)c3ccc(CN4CCN(Cc5ccccc5Cl)CC4)cc23)n1]

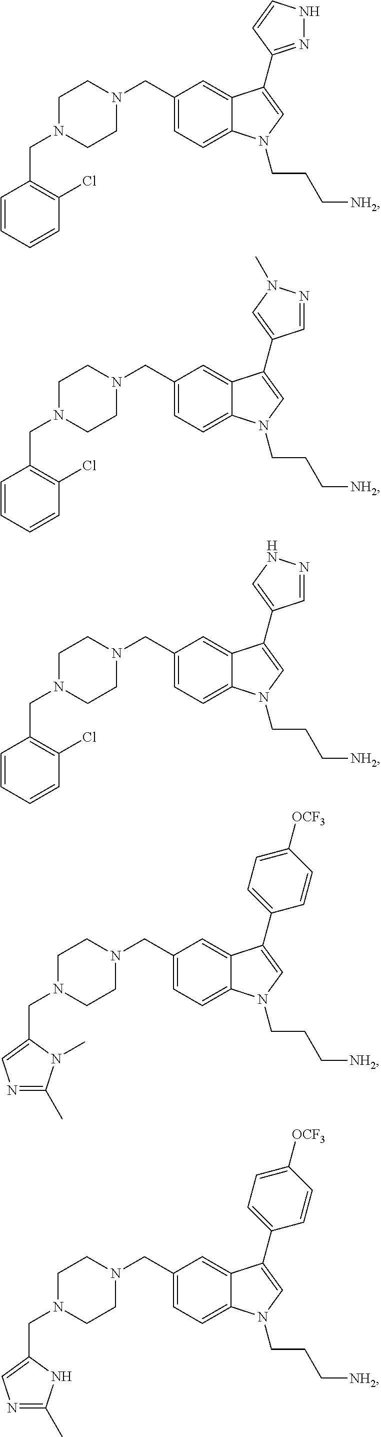 [SMILES: NCCCn1cc(-c2cc[nH]n2)c2cc(CN3CCN(Cc4ccccc4Cl)CC3)ccc21 ; Cn1cc(-c2cn(CCCN)c3ccc(CN4CCN(Cc5ccccc5Cl)CC4)cc23)cn1 ; NCCCn1cc(-c2cn[nH]c2)c2cc(CN3CCN(Cc4ccccc4Cl)CC3)ccc21 ; Cc1ncc(CN2CCN(Cc3ccc4c(c3)c(-c3ccc(OC(F)(F)F)cc3)cn4CCCN)CC2)n1C ; Cc1ncc(CN2CCN(Cc3ccc4c(c3)c(-c3ccc(OC(F)(F)F)cc3)cn4CCCN)CC2)[nH]1]

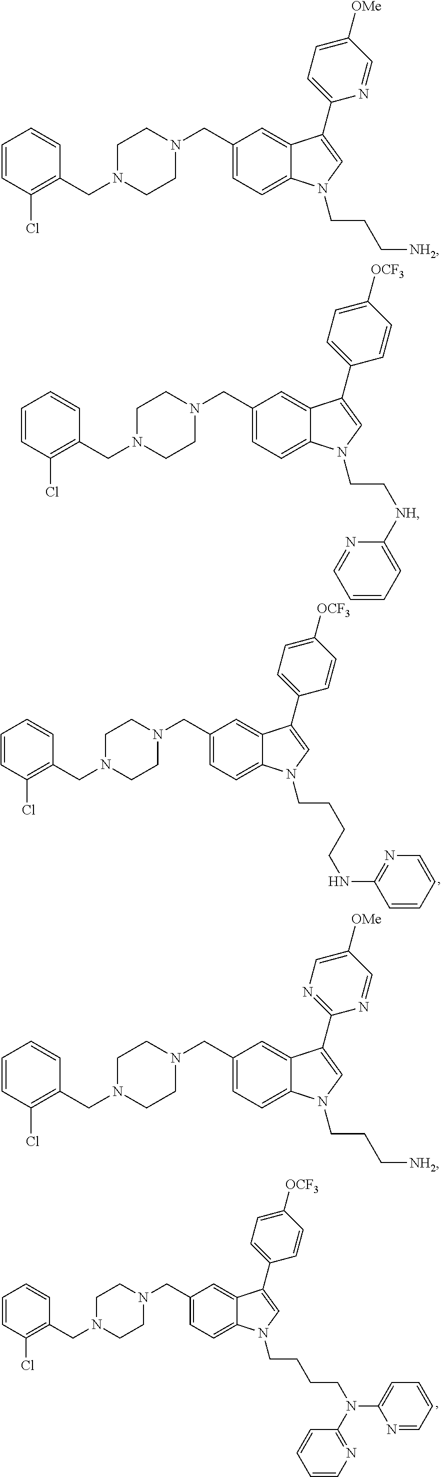

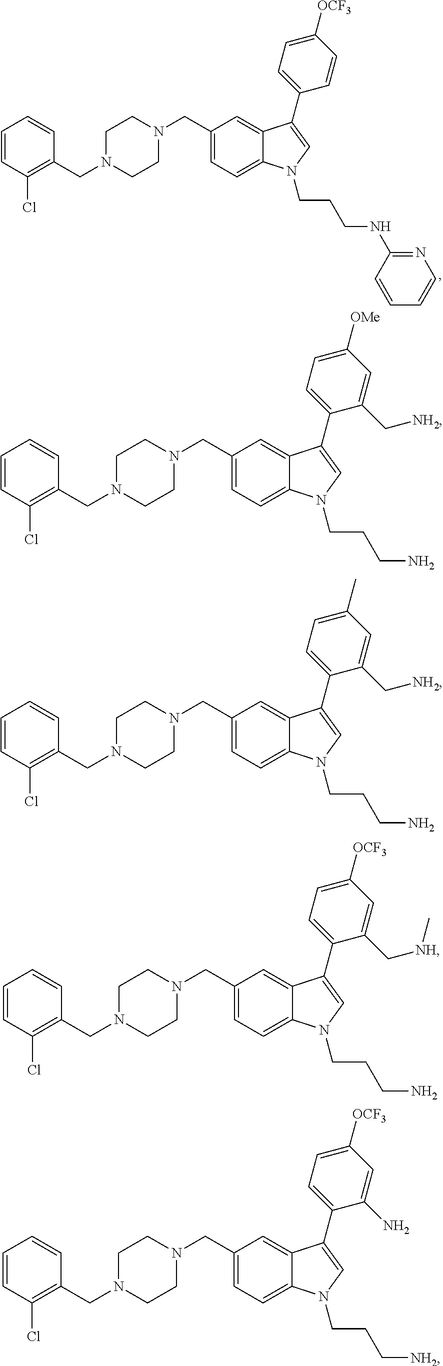

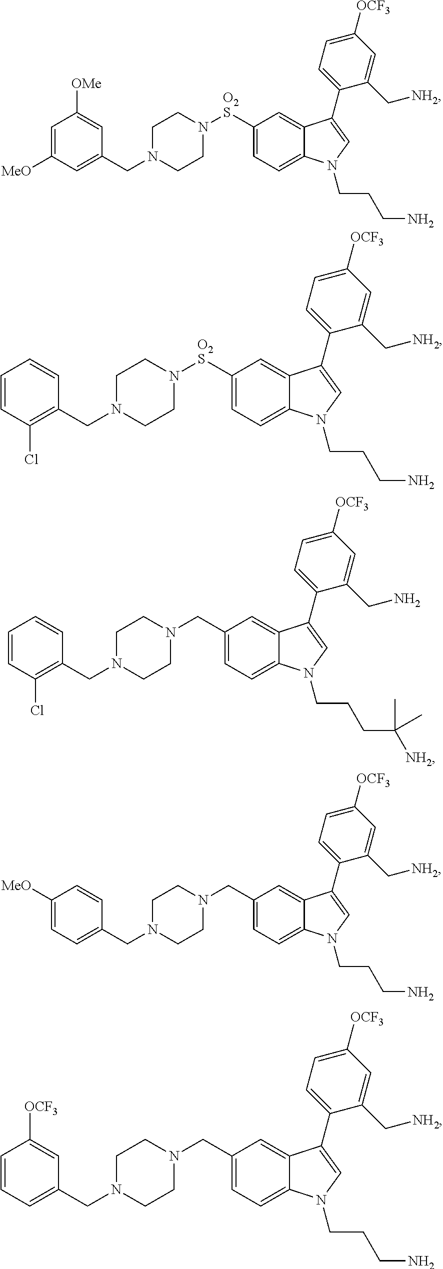

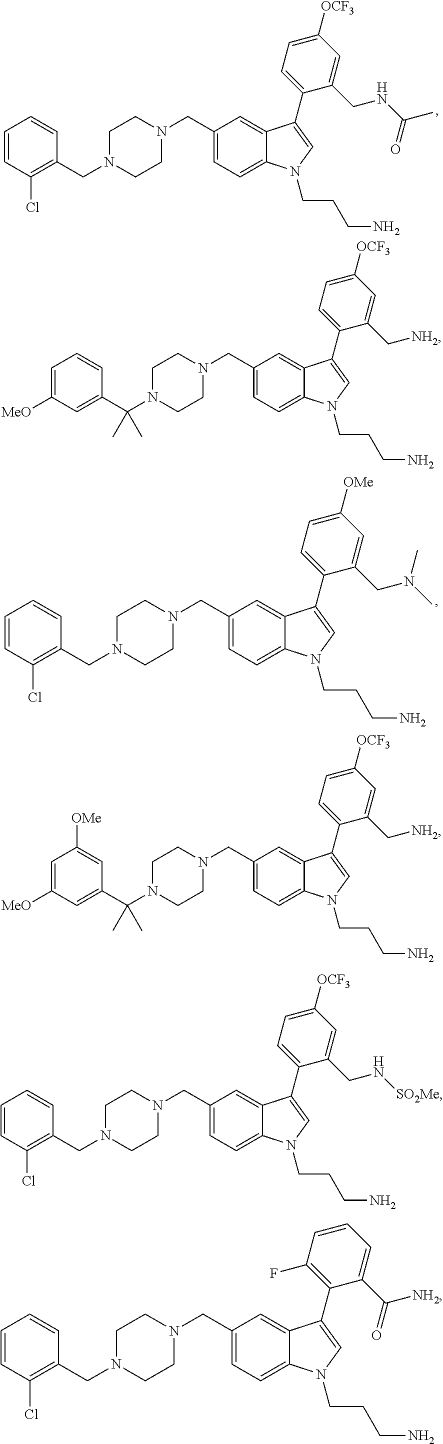

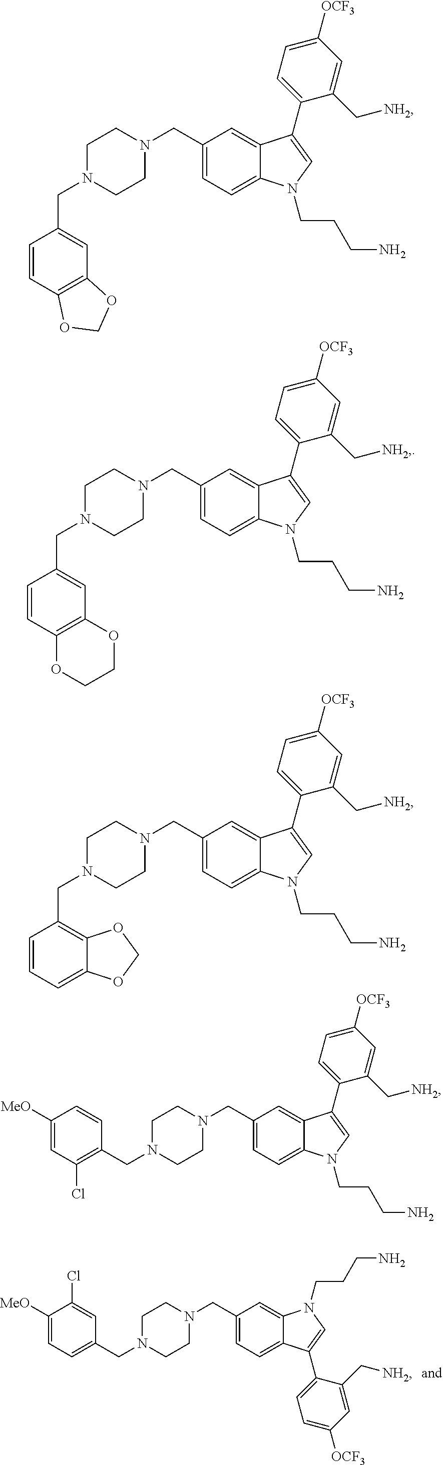

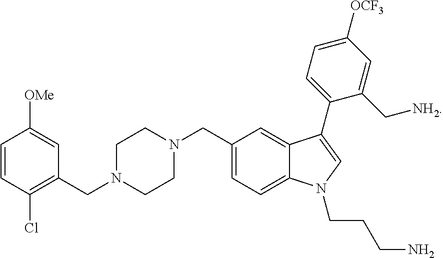 [SMILES: COc1ccc(Cl)c(CN2CCN(Cc3ccc4c(c3)c(-c3ccc(OC(F)(F)F)cc3CN)cn4CCCN)CC2)c1]

145. A pharmaceutical composition comprising a compound according to claim 84, or a pharmaceutically acceptable salt, or solvate thereof.

146. (canceled)

147. A method for treating or ameliorating the effects of a disease associated with altered Ras signaling, the method comprising administering to a subject in need thereof a pharmaceutical composition, wherein the pharmaceutical composition comprises the compound of claim 84, or a pharmaceutically acceptable salt, or solvate thereof.

148. The method of claim 147, wherein the disease is a cancer, a neurological disorder, a metabolic disorder, an immunological disorder, an inflammatory disorder, or a developmental disorder.

149.-151. (canceled)

152. A method for reducing or depleting a population of cancer cells, the method comprising administering a pharmaceutical composition to a subject in need thereof, wherein the pharmaceutical composition comprises the compound of claim 84, or a pharmaceutically acceptable salt, or solvate thereof.

153.-157. (canceled)
